(19) United States
(12) Patent Application Publication
(10) Pub. No.: US 2025/0092374 A1
(43) Pub. Date: Mar. 20, 2025

(54) EVOLVED CAS9 PROTEINS FOR GENE EDITING

(71) Applicant: President and Fellows of Harvard College, Cambridge, MA (US)

(72) Inventors: David R. Liu, Cambridge, MA (US); Johnny Hao Hu, Cambridge, MA (US)

(73) Assignee: President and Fellows of Harvard College, Cambridge, MA (US)

(21) Appl. No.: 18/732,559

(22) Filed: Jun. 3, 2024

Related U.S. Application Data
(63) Continuation of application No. 15/770,076, filed on Apr. 20, 2018, now Pat. No. 12,043,852.
(60) Provisional application No. 62/408,686, filed on Oct. 14, 2016.
(60) Provisional application No. 62/398,490, filed on Sep. 22, 2016.
(60) Provisional application No. 62/370,700, filed on Aug. 3, 2016.
(60) Provisional application No. 62/357,352, filed on Jun. 30, 2016.
(60) Provisional application No. 62/357,332, filed on Jun. 30, 2016.
(60) Provisional application No. 62/322,178, filed on Apr. 13, 2016.
(60) Provisional application No. 62/311,763, filed on Mar. 22, 2016.
(60) Provisional application No. 62/279,346, filed on Jan. 15, 2016.
(60) Provisional application No. 62/245,828, filed on Oct. 23, 2015.

(51) Int. Cl. C12N 9/22; C07K 14/32; C12N 9/24; C12N 9/78; C12N 15/11; C12N 15/90

(52) U.S. Cl. CPC C12N 9/22; C07K 14/32; C12N 9/2497; C12N 9/78; C12N 15/11; C12N 15/907; C12Y 305/04005; C07K 2319/00; C07K 2319/09; C07K 2319/80; C12N 2310/20; C12N 2800/80

(57) ABSTRACT

Some aspects of this disclosure provide strategies, systems, reagents, methods, and kits that are useful for engineering Cas9 and Cas9 variants that have increased activity on target sequences that do not contain the canonical PAM sequence. In some embodiments, fusion proteins comprising such Cas9 variants and nucleic acid editing domains, e.g., deaminase domains, are provided.

### RELATED APPLICATIONS

This application is a national stage filing under 35 U.S.C. § 371 of international PCT application, PCT/US2016/058345, filed Oct. 22, 2016, which claims priority under 35 U.S.C. § 119 (c) to U.S. provisional patent applications, U.S. Ser. No. 62/245,828 filed Oct. 23, 2015, U.S. Ser. No. 62/279,346 filed Jan. 15, 2016, U.S. Ser. No. 62/311,763 filed Mar. 22, 2016, U.S. Ser. No. 62/322,178 filed Apr. 13, 2016, U.S. Ser. No. 62/357,352 filed Jun. 30, 2016, U.S. Ser. No. 62/370,700 filed Aug. 3, 2016, U.S. Ser. No. 62/398,490 filed Sep. 22, 2016, U.S. Ser. No. 62/408,686 filed Oct. 14, 2016, and U.S. Ser. No. 62/357,332 filed Jun. 30, 2016; each of which is incorporated herein by reference.

### REFERENCE TO A SEQUENCE LISTING SUBMITTED AS A TEXT FILE VIA EFS-WEB

The present application contains a Sequence Listing which has been submitted in ASCII format via EFS-Web and is hereby incorporated by reference in its entirety. Said ASCII copy, created on Jun. 28, 2022, is named H082470224US02-SUBSEQ-EPG and is 3,977,840 bytes in size.

### BACKGROUND OF THE INVENTION

Targeted editing of nucleic acid sequences, for example, the targeted cleavage or the targeted introduction of a specific modification into genomic DNA, is a highly promising approach for the study of gene function and also has the potential to provide new therapies for human genetic diseases.¹ An ideal nucleic acid editing technology possesses three characteristics: (1) high efficiency of installing the desired modification; (2) minimal off-target activity; and (3) the ability to be programmed to edit precisely any site in a given nucleic acid, e.g., any site within the human genome.² Current genome engineering tools, including engineered zinc finger nucleases (ZFNs),³ transcription activator like effector nucleases (TALENs),⁴ and most recently, the RNA-guided DNA endonuclease Cas9,5 effect sequence-specific DNA cleavage in a genome. This programmable cleavage can result in mutation of the DNA at the cleavage site via non-homologous end joining (NHEJ) or replacement of the DNA surrounding the cleavage site via homology-directed repair (HDR).^{6,7}

One drawback of the current technologies is that both NHEJ and HDR are stochastic processes that typically result in modest gene editing efficiencies as well as unwanted gene alterations that can compete with the desired alteration.⁸ Since many genetic diseases in principle can be treated by effecting a specific nucleotide change at a specific location in the genome (for example, a C to T change in a specific codon of a gene associated with a disease),⁹ the development of a programmable way to achieve such precise gene editing would represent both a powerful new research tool, as well as a potential new approach to gene editing-based human therapeutics.

Another drawback of current genome engineering tools is that they are limited with respect to the DNA sequences that can be targeted. When using ZNFs or TALENS, a new protein must be generated for each individual target sequence. While Cas9 can be targeted to virtually any target sequence by providing a suitable guide RNA, Cas9 technology is still limited with respect to the sequences that can be targeted by a strict requirement for a protospacer-adjacent motif (PAM), typically of the nucleotide sequence 5′-NGG-3′, that must be present immediately adjacent to the 3′-end of the targeted DNA sequence in order for the Cas9 protein to bind and act upon the target sequence. The PAM requirement thus limits the sequences that can be efficiently targeted by Cas9 proteins.

### SUMMARY OF THE INVENTION

Significantly, 80-90% of protein mutations responsible for human disease arise from the substitution, deletion, or insertion of only a single nucleotide.⁶ Most current strategies for single-base gene correction include engineered nucleases (which rely on the creation of double-strand breaks, DSBs, followed by stochastic, inefficient homology-directed repair, HDR), and DNA-RNA chimeric oligonucleotides.²² The latter strategy involves the design of a RNA/DNA sequence to base pair with a specific sequence in genomic DNA except at the nucleotide to be edited. The resulting mismatch is recognized by the cell's endogenous repair system and fixed, leading to a change in the sequence of either the chimera or the genome. Both of these strategies suffer from low gene editing efficiencies and unwanted gene alterations, as they are subject to both the stochasticity of HDR and the competition between HDR and non-homologous end-joining, NHEJ.²³⁻²⁵ HDR efficiencies vary according to the location of the target gene within the genome,²⁶ the state of the cell cycle,²⁷ and the type of cell/tissue.²⁸ The development of a direct, programmable way to install a specific type of base modification at a precise location in genomic DNA with enzyme-like efficiency and no stochasticity therefore represents a powerful new approach to gene editing-based research tools and human therapeutics.

The clustered regularly interspaced short palindromic repeat (CRISPR) system is a recently discovered prokaryotic adaptive immune system¹⁰ that has been modified to enable robust and general genome engineering in a variety of organisms and cell lines.¹¹ CRISPR-Cas (CRISPR-associated) systems are protein-RNA complexes that use an RNA molecule (sgRNA) as a guide to localize the complex to a target DNA sequence via base-pairing.¹² In the natural systems, a Cas protein then acts as an endonuclease to cleave the targeted DNA sequence.¹³ The target DNA sequence must be both complementary to the sgRNA and also contain a “protospacer-adjacent motif” (PAM) at the 3′-end of the complementary region in order for the system to function.¹⁴ The requirement for a PAM sequence limits the use of Cas9 technology, since not all desired targeted sequences include a PAM sequence at the 3′-end and thus cannot efficiently be targeted by wild-type Cas9 proteins.

Provided herein are novel Cas9 variants that exhibit activity on target sequences that do not include the canonical PAM sequence (5′-NGG-3′, where N is any nucleotide) at the 3′-end. Such Cas9 variants are not restricted to target sequences that include the canonical PAM sequence at the 3′-end.

Among the known Cas proteins, Streptococcus pyogenes Cas9 has been mostly widely used as a tool for genome engineering.¹⁵ This Cas9 protein is a large, multi-domain protein containing two distinct nuclease domains. Point mutations can be introduced into Cas9 to abolish nuclease activity, resulting in a dead Cas9 (dCas9) that still retains its ability to bind DNA in a sgRNA-programmed manner.¹⁶ In principle, such Cas9 variants, when fused to another protein or domain, can target that protein to virtually any DNA sequence simply by co-expression with an appropriate sgRNA. Thus, this disclosure also contemplates fusion proteins comprising such Cas9 variants and a DNA modifying domain (e.g., a deaminase, a nuclease, a nickase, a recombinase, a methyltransferase, a methylase, an acetylase, an acetyltransferase, a transcriptional activator, or a transcriptional repressor domain), as well as the use of such fusion proteins in correcting mutations in a genome (e.g., the genome of a human subject) that are associated with disease, or generating mutations in a genome (e.g., the human genome) to decrease or prevent expression of a gene.

In some embodiments, any of the Cas9 proteins provided herein may be fused to a protein that has an enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity, deacetylase activity, kinase activity, phosphatase activity, ubiquitin ligase activity or deubiquitinating activity.

In some embodiments, any of the Cas9 proteins provided herein may be fused to a protein that has an enzymatic activity. In some embodiments, the enzymatic activity modifies a polypeptide associated with DNA (e.g. a histone). In some embodiments, the enzymatic activity is methyltransferase activity, demethylase activity, acetyltransferase activity, deacetylase activity, kinase activity, phosphatase activity, ubiquitin ligase activity (i.e., ubiquitination activity), deubiquitinating activity, adenylation activity, deadenylation activity, SUMOylating activity, deSUMOylating activity, ribosylation activity, deribosylation activity, myristoylation activity, demyristoylation activity glycosylation activity (e.g., from O-GlcNAc transferase) or deglycosylation activity. The enzymatic activities listed herein catalyze covalent modifications to proteins. Such modifications are known in the art to alter the stability or activity of the target protein (e.g., phosphorylation due to kinase activity can stimulate or silence protein activity depending on the target protein). Of particular interest as protein targets are histones. Histone proteins are known in the art to bind DNA and form complexes known as nucleosomes. Histones can be modified (e.g., by methylation, acetylation, ubuitination, phosphorylation) to elicit structural changes in the surrounding DNA, thus controlling the accessibility of potentially large portions of DNA to interacting factors such as transcription factors, polymerases and the like. A single histone can be modified in many different ways and in many different combinations (e.g., trimethylation of lysine 27 of histone 3, H3K27, is associated with DNA regions of repressed transcription while trimethylation of lysine 4 of histone 3, H3K4, is associated with DNA regions of active transcription). Thus, a site-directed modifying polypeptide with histone-modifying activity finds use in the site specific control of DNA structure and can be used to alter the histone modification pattern in a selected region of target DNA. Such methods find use in both research and clinical applications.

In some embodiments, the deaminase domain catalyzes the removal of an amine group from a molecule. In further embodiments, cytidine deaminase domains deaminate cytosine to yield uracil. In other embodiments, the nuclease domain has enzymatic activity and may cleave phosphodiester bonds between the nucleotide subunits of nucleic acids. In some embodiments, recombinase domains, which recombine specific sequences of DNA, may be used to manipulate the structure of genomes and to control gene expression. In further embodiments, methylase domains may be utilized to methylate their respective substrates, while acetylase domains may be used to acetylate their respective substrates. In other embodiments, acetyltransferase domains may be used to transfer an acetyl group. Examples of acetyltransferase molecules include, but are not limited to, histone acetyltransferases (e.g., CBP histone acetyltransferase), choline acetyltransferase, chloramphenicol acetytransferase, serotonic N-acetyltransferase, NatA acetyltransferase, and NatB acetyltransferase. The disclosure also contemplates transcriptional activator and transcriptional repressor domains. Transcriptional activator domains are regions of a transcription factor which may activate transcription from a promoter through an interaction or multiple interactions with a DNA binding domain, general transcription factors, and RNA polymerase. Transcriptional repressor domains are regions of a transcription factor which may repress transcription from a protomer through an interaction or multiple interactions with a DNA binding domain, general transcription factors, and RNA polymerase.

The potential of the Cas9 system for genome engineering is immense. Its unique ability to bring proteins to specific sites in a genome programmed by the sgRNA can be developed into a variety of site-specific genome engineering tools beyond nucleases, including transcriptional activators, transcriptional repressors, histone-modifying proteins, integrases, deaminases, and recombinases.¹¹ Some of these potential applications have recently been implemented through dCas9 fusions with transcriptional activators to afford RNA-guided transcriptional activators,^{17,18} transcriptional repressors,^{16,19,20} and chromatin modification enzymes.²¹ Simple co-expression of these fusions with a variety of sgRNAs results in specific expression of the target genes. These seminal studies have paved the way for the design and construction of readily programmable sequence-specific effectors for the precise manipulation of genomes.

Some aspects of this disclosure provide strategies, systems, proteins, nucleic acids, compositions, cells, reagents, methods, and kits that are useful for the targeted binding, editing, and/or cleaving of nucleic acids, including editing a single site within a subject's genome, e.g., a human subject's genome. In some embodiments, recombinant Cas9 proteins are provided that comprise at least one, at least two, at least three, at least four, at least five, at least six, at least seven, at least eight, at least nine, or at least ten mutations as compared to a naturally occurring Cas9 protein, and that exhibit activity on target sequences that do not include the canonical PAM (5′-NGG-3′, where N is any nucleotide) at the 3′-end. Examples of such Cas9 protein mutations are given in Tables 3, 5, 8, and 9. In some embodiments, fusion proteins of Cas9 and nucleic acid editing enzymes or enzymatic domains, e.g., deaminase domains, are provided. In some embodiments, methods for targeted nucleic acid binding, editing, and/or cleaving are provided. In some embodiments, reagents and kits for the generation of targeted nucleic acid binding, editing, and/or cleaving proteins, e.g., fusion proteins of Cas9 variants and nucleic acid editing enzymes or domains, are provided.

Some aspects of this disclosure provide recombinant Cas9 proteins comprising an amino acid sequence that is at least 80%, at least 85%, at least 90%, at least 92%, at least 95%, at least 96%, at least 97%, at least 98%, at least 99%, or at least 99.5% identical to the amino acid sequence of Cas9 as provided by any of the sequences set forth in SEQ ID NOs: 9-262, wherein the amino acid sequence of the Cas9 protein comprises at least one, at least two, at least three, at least four, at least five, at least six, or at least seven mutations in an amino acid residue selected from the group consisting of amino acid residues 122, 137, 182, 262, 294, 409, 480, 543, 660, 694, 1219, and 1329 of S. pyogenes Cas9 having the amino acid sequence provided in SEQ ID NO: 9, or in a corresponding amino acid residue in any of the amino acid sequences provided in SEQ ID NOs: 10-262. In some embodiments, the recombinant Cas9 protein comprises a RuvC and an HNH domain. In some embodiments, the amino acid sequence of the recombinant Cas9 protein is not identical to the amino acid sequence of a naturally occurring Cas9 protein. In some embodiments, the amino acid sequence of the Cas9 protein comprises at least one, at least two, at least three, at least four, at least five, at least six, or at least seven mutations selected from the group consisting of X262T, X294R, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 protein comprises at least one, at least two, at least three, at least four, at least five, at least six, or at least seven mutations selected from the group consisting of A262T, K294R, S409I, E480K, E543D, M694I, and E1219V of the amino acid sequence provided in SEQ ID NO: 9, or a corresponding mutation in any of the amino acid sequences provided in SEQ ID NOs: 10-262.

Other aspects of this disclosure provide recombinant Cas9 proteins comprising an amino acid sequence that is at least 80%, at least 85%, at least 90%, at least 92%, at least 95%, at least 96%, at least 97%, at least 98%, at least 99%, or at least 99.5% identical to the amino acid sequence of Cas9 as provided by any of the sequences set forth in SEQ ID NOs: 10-262, wherein the amino acid sequence of the Cas9 protein comprises at least one, at least two, at least three, at least four, at least five, at least six, or at least seven mutations in an amino acid residue selected from the group consisting of amino acid residues 262, 267, 294, 405, 409, 480, 543, 694, 1219, 1224, 1256, and 1362 of the amino acid sequence provided in SEQ ID NO: 9, or in a corresponding amino acid residue in any of the amino acid sequences provided in SEQ ID NOS: 10-262; and wherein the amino acid sequence of the recombinant Cas9 protein is not identical to the amino acid sequence of a naturally occurring Cas9 protein. In some embodiments, the Cas9 protein comprises a RuvC and an HNH domain. In some embodiments, the amino acid sequence of the Cas9 protein comprises at least one, at least two, at least three, at least four, at least five, at least six, or at least seven mutations selected from the group consisting of X262T, X267G, X294R, X405I, X409I, X480K, X543D, X694I, X1219V, X1224K, and X1256K of the amino acid sequence provided in SEQ ID NO: 9, or a corresponding mutation in any of the amino acid sequences provided in SEQ ID NOs: 10-262, wherein X represents any amino acid. In some embodiments, the amino acid sequence of the Cas9 protein comprises at least one, at least two, at least three, at least four, at least five, at least six, or at least seven mutations selected from the group consisting of A262T, S267G, K294R, F405I, S409I, E480K, E543D, M694I, E1219V, N1224K, and Q1256K of the amino acid sequence provided in SEQ ID NO: 9, or a corresponding mutation in any of the amino acid sequences provided in in SEQ ID NOs: 10-262.

It should be appreciated that any of the amino acid mutations described herein, (e.g., A262T) from a first amino acid residue (e.g., A) to a second amino acid residue (e.g., T) may also include mutations from the first amino acid residue to an amino acid residue that is similar to (e.g., conserved) the second amino acid residue. For example, a mutation of an alanine to a threonine (e.g., a A262T mutation) may also be a mutation from an alanine to an amino acid that is similar in size and chemical properties to a threonine, for example, serine. Additional similar amino acid pairs include, but are not limited to, the following: phenylalanine and tyrosine; asparagine and glutamine; methionine and cysteine; aspartic acid and glutamic acid; and arginine and lysine. The skilled artisan would recognize that such conservative amino acid substitutions will likely have minor effects on protein structure and are likely to be well tolerated without compromising function. In some embodiments, any of the amino acid mutations provided herein from one amino acid to a threonine may be an amino acid mutation to a serine. In some embodiments, any of the amino acid mutations provided herein from one amino acid to an arginine may be an amino acid mutation to a lysine. In some embodiments, any of the amino acid mutations provided herein from one amino acid to an isoleucine may be an amino acid mutation to an alanine, valine, methionine, or leucine. In some embodiments, any of the amino acid mutations provided herein from one amino acid to a lysine may be an amino acid mutation to an arginine. In some embodiments, any of the amino acid mutations provided herein from one amino acid to an aspartic acid may be an amino acid mutation to a glutamic acid or asparagine. In some embodiments, any of the amino acid mutations provided herein from one amino acid to a valine may be an amino acid mutation to an alanine, isoleucine, methionine, or leucine. In some embodiments, any of the amino acid mutations provided herein from one amino acid to a glycine may be an amino acid mutation to an alanine. It should be appreciated, however, that additional conserved amino acid residues would be recognized by the skilled artisan and any of the amino acid mutations to other conserved amino acid residues are also within the scope of this disclosure.

In some embodiments, the Cas9 protein is a Cas9 domain of a fusion protein. In some embodiments, the amino acid sequence of the Cas9 protein comprises an X1219V mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X1219A, X1219I, X1219M, or X1219L.

In some embodiments, the amino acid sequence of the Cas9 protein comprises an E1219V mutation of the amino acid sequence provided in SEQ ID NO: 9, or a corresponding mutation in any of the amino acid sequences provided in SEQ ID NOs: 10-262. In some embodiments, the mutation is E1219A, E1219I, E1219M or E1219L.

In some embodiments, the amino acid sequence of the Cas9 protein comprises an X480K mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X480R.

In some embodiments, the amino acid sequence of the Cas9 protein comprises an E480K mutation of the amino acid sequence provided in SEQ ID NO: 9, or a corresponding mutation in any of the amino acid sequences provided in SEQ ID NOs: 10-262. In some embodiments, the mutation is E480R.

In some embodiments, the amino acid sequence of the Cas9 protein comprises an X543D mutation of the amino acid sequence provided in SEQ ID NO: 9, or a corresponding mutation in any of the amino acid sequences provided in SEQ ID NOs: 10-262, wherein X represents any amino acid. In some embodiments, the mutation is X543N.

In some embodiments, the amino acid sequence of the Cas9 protein comprises an E543D mutation of the amino acid sequence provided in SEQ ID NO: 9, or a corresponding mutation in any of the amino acid sequences provided in SEQ ID NOs: 10-262. In some embodiments, the mutation is E543N.

In some embodiments, the amino acid sequence of the Cas9 protein comprises the mutations X480K, X543D, and X1219V of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X262T, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X294R, X480K, X543D, X1219V, X1256K, and X1362P of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X294R, X480K, X543D, X1219V, and X1256K of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X267G, X294R, X480K, X543D, X1219V, X1224K, and X1256K of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X262T, X405I, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations E480K, E543D, and E1219V of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises the mutations A262T, S409I, E480K, E543D, M694I, and E1219V of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises the mutations K294R, E480K, E543D, E1219V, Q1256K, and L1362P of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises the mutations K294R, E480K, E543D, E1219V, and Q1256K of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises the mutations S267G, K294R, E480K, E543D, E1219V, N1224K, and Q1256K of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises the mutations A262T, F405I, S409I, E480K, E543D, M694I, and E1219V of the amino acid sequence provided in SEQ ID NO: 9, or a corresponding mutation in any of the amino acid sequences provided in SEQ ID NOs: 10-262.

The HNH nuclease domain of Cas9 functions to cleave the DNA strand complementary to the guide RNA (gRNA). Its active site consists of a ββα-metal fold, and its histidine 840 activates a water molecule to attack the scissile phosphate, which is more electrophilic due to coordination with a magnesium ion, resulting in cleavage of the 3′-5′ phosphate bond. In some embodiments, the amino acid sequence of the HNH domain is at least 80%, at least 85%, at least 90%, at least 92%, at least 95%, at least 96%, at least 97%, at least 98%, at least 99%, or at least 99.5% identical to the amino acid sequence of the HNH domain of any of SEQ ID NOs: 9-262. In some embodiments, the amino acid sequence of the HNH domain is identical to the amino acid sequence of the HNH domain of any of SEQ ID NOs: 9-262.

The RuvC domain of Cas9 cleaves the non-target DNA strand. It is encoded by sequentially disparate sites which interact in the tertiary structure to form the RuvC cleavage domain and consists of an RNase H fold structure. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 9-262. In some embodiments, the amino acid sequence of the RuvC domain is identical to the amino acid sequence of the RuvC domain of any of SEQ ID NOs: 9-262.

In some embodiments, the Cas9 protein comprises one or more mutations that affects (e.g., inhibits) the ability of Cas9 to cleave one or both strands of a DNA duplex. In some embodiments, the Cas9 protein comprises a D10A and/or a H840A mutation of the amino acid sequence provided in SEQ ID NO: 9, or a corresponding mutation in any of the amino acid sequences provided in SEQ ID NOs: 10-262. In some embodiments, the Cas9 protein comprises a D10X₁ and/or a H840X₂ mutation of the amino acid sequence provided in SEQ ID NO: 9, or a corresponding mutation in any of the amino acid sequences provided in SEQ ID NOs: 10-262, wherein X1 is any amino acid except for D, and X₂ is any amino acid except for H. In some embodiments, the Cas9 protein comprises an D10A mutation of the amino acid sequence provided in SEQ ID NO: 9, or a corresponding mutation in any of the amino acid sequences provided in SEQ ID NOs: 10-262. In some embodiments, the Cas9 protein comprises an H at amino acid residue 840 of the amino acid sequence provided in SEQ ID NO: 9, or a corresponding residue in any of the amino acid sequences provided in SEQ ID NOs: 10-262. In some embodiments, the Cas9 protein comprises an H840A mutation of the amino acid sequence provided in SEQ ID NO: 9, or a corresponding mutation in any of the amino acid sequences provided in SEQ ID NOs: 10-262. In some embodiments, the Cas9 protein comprises a D at amino acid residue 10 of the amino acid sequence provided in SEQ ID NO: 9, or a corresponding residue in any of the amino acid sequences provided in SEQ ID NOs: 10-262.

In some embodiments, the Cas9 protein of the present disclosure exhibits activity, for example, increased binding, on a target sequence that does not include the canonical PAM sequence (5′-NGG-3′) at its 3′-end as compared to Streptococcus pyogenes Cas9 as provided by SEQ ID NO: 9.

Some aspects of this disclosure provide recombinant Cas9 proteins comprising an amino acid sequence that is at least 90% identical to the amino acid sequence of Streptococcus pyogenes Cas9 as provided by SEQ ID NO: 9 wherein the amino acid sequence of the Cas9 protein comprises at least one, at least two, at least three, at least four, at least five, at least six, or at least seven mutations selected from the group consisting of amino acid residues 262, 267, 294, 405, 409, 480, 543, 694, 1219, 1224, 1256, and 1362 of the amino acid sequence provided in SEQ ID NO: 9, wherein the amino acid sequence of the recombinant Cas9 protein is not identical to the amino acid sequence of a naturally occurring Cas9 protein, and wherein the recombinant Cas9 protein exhibits increased activity on a target sequence that does not comprise the canonical PAM sequence (5′-NGG-3′) at its 3′ end as compared to Streptococcus pyogenes Cas9 as provided in SEQ ID NO: 9. In some embodiments, the Streptococcus pyogenes Cas9 comprises a RuvC and an HNH domain. In other embodiments, the Cas9 protein comprises at least one, at least two, at least three, at least four, at least five, at least six, or at least seven mutations selected from the group consisting of amino acid residues 122, 137, 182, 262, 294, 409, 480, 543, 660, 694, 1219, and 1329 of the amino acid sequence provided in SEQ ID NO: 9

As one example, the Cas9 protein may exhibit increased binding to the target sequence, may exhibit increased nuclease activity at the target sequence, or may exhibit an increase in other activities, depending on whether the Cas 9 protein is fused to an additional domain, such as an enzyme that has enzymatic activity. In some embodiments, the enzymatic activity modifies a target DNA. In some embodiments, the enzymatic activity is nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. In some cases, the enzymatic activity is nuclease activity. In some cases, the nuclease activity introduces a double strand break in the target DNA. In some cases, the enzymatic activity modifies a target polypeptide associated with the target DNA. In some cases, the enzymatic activity is methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity. In some cases, the target polypeptide is a histone and the enzymatic activity is methyltransferase activity, demethylase activity, acetyltransferase activity, deacetylase activity, kinase activity, phosphatase activity, ubiquitin ligase activity or deubiquitinating activity.

In some embodiments, any of the Cas9 protein is fused to a protein that has an enzymatic activity. In some embodiments, the enzymatic activity modifies a polypeptide associated with DNA (e.g. a histone). In some embodiments, the enzymatic activity is methyltransferase activity, demethylase activity, acetyltransferase activity, deacetylase activity, kinase activity, phosphatase activity, ubiquitin ligase activity (i.e., ubiquitination activity), deubiquitinating activity, adenylation activity, deadenylation activity, SUMOylating activity, deSUMOylating activity, ribosylation activity, deribosylation activity, myristoylation activity, demyristoylation activity glycosylation activity (e.g., from O-GlcNAc transferase) or deglycosylation activity. The enzymatic activities listed herein catalyze covalent modifications to proteins. Such modifications are known in the art to alter the stability or activity of the target protein (e.g., phosphorylation due to kinase activity can stimulate or silence protein activity depending on the target protein). Of particular interest as protein targets are histones. Histone proteins are known in the art to bind DNA and form complexes known as nucleosomes. Histones can be modified (e.g., by methylation, acetylation, ubuitination, phosphorylation) to elicit structural changes in the surrounding DNA, thus controlling the accessibility of potentially large portions of DNA to interacting factors such as transcription factors, polymerases and the like. A single histone can be modified in many different ways and in many different combinations (e.g., trimethylation of lysine 27 of histone 3, H3K27, is associated with DNA regions of repressed transcription while trimethylation of lysine 4 of histone 3, H3K4, is associated with DNA regions of active transcription). Thus, a site-directed modifying polypeptide with histone-modifying activity finds use in the site specific control of DNA structure and can be used to alter the histone modification pattern in a selected region of target DNA. Such methods find use in both research and clinical applications.

In some embodiments, the Cas9 protein exhibits activity on a target sequence having a 3′ end that is not directly adjacent to, or does not have the canonical PAM sequence (5′-NGG-3′), that is at least 2-fold, at least 3-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes Cas9 as provided by SEQ ID NO: 9 on the same target sequence.

In some embodiments, the 3′-end of the target sequence is directly adjacent to an AGC, GAG, TTT, GTG, CAA CAC, GAT, TAA, ACG, CGA, or CGT sequence.

In some embodiments, the Cas9 protein activity is measured by a nuclease assay or a nucleic acid binding assay, which are known in the art and would be apparent to the skilled artisan. As provided herein, the Cas9 protein may be fused to one or more domains that confer an activity to the protein, such as a nucleic acid editing activity (e.g., deaminase activity or transcriptional activation activity), which may be measured (e.g., by a deaminase assay or transcriptional activation assay). In some embodiments, the Cas9 protein is fused to a deaminase domain and its activity may be measured using a deaminase assay. In some embodiments, the Cas9 protein is fused to a transcriptional activation domain and its activity may be measured using a transcriptional activation assay, for example, reporter activation assay where the reporter, e.g., GFP or luciferase, among others, is expressed in response to Cas9 binding to a target sequence.

In some embodiments, the amino acid sequence of the Cas9 protein comprises any of the mutations provided herein. For example, in some embodiments, the amino acid sequence of the Cas9 protein comprises at least one, at least two, at least three, at least four, at least five, at least six, or at least seven mutations selected from the group consisting of X262T, X267G, X294R, X405I, X409I, X480K, X543D, X694I, X1219V, X1224K, X1256K, and X1362P of the amino acid sequence provided in SEQ ID NO: 9, or a corresponding mutation in any of the amino acid sequences provided in SEQ ID NOs: 10-262, wherein X represents any amino acid. In other embodiments, the mutations may be A262T, S267G, K294R, F405I, S409I, E480K, E543D, M694I, E1219V, N1224K, Q1256K, and L1362P of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises any of the mutations provided herein. For example, in some embodiments, the amino acid sequence of the Cas9 protein comprises at least one, at least two, at least three, at least four, at least five, at least six, or at least seven mutations selected from the group consisting of X262T, X294R, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid. In other embodiments, the mutations may be A262T, K294R, S409I, E480K, E543D, M694I, or E1219V of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises an X1219V mutation or an E1219V mutation of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises an X480K mutation or an E480K mutation of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises an X543D mutation or a E543D mutation of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 comprises the mutations X480K, X543D, and X1219V; or the mutations E480K, E543D, and E1219V of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 comprises the mutations X262T, X409I, X480K, X543D, X694I, and X1219V; or the mutations A262T, S409I, E480K, E543D, M694I, and E1219V of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X294R, X480K, X543D, X1219V, X1256K, and X1362P; or the mutations K294R, E480K, E543D, E1219V, Q1256K, and L1362P of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X294R, X480K, X543D, X1219V, and X1256K, or mutations K294R, E480K, E543D, E1219V, and Q1256K of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X267G, X294R, X480K, X543D, X1219V, X1224K, and X1256K; or the mutations S267G, K294R, E480K, E543DE1219V, N1224K, and Q1256K of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the Cas9 protein comprises the mutations X262T, X405I, X409I, X480K, X543D, X694I, and X1219V; or the mutations A262T, F405I, S409I, E480K, E543D, M694I, and E1219V of the amino acid sequence provided in SEQ ID NO: 9, or a corresponding mutation in any of the amino acid sequences provided in SEQ ID NOs: 10-262, wherein X represents any amino acid.

In some embodiments, the amino acid sequence of the HNH domain is at least 80%, at least 85%, at least 90%, at least 92%, at least 95%, at least 96%, at least 97%, at least 98%, at least 99%, or at least 99.5% identical to the amino acid sequence of the HNH domain of any of SEQ ID NOs: 9-262. In some embodiments, the amino acid sequence of the HNH domain is identical to the amino acid sequence of any of the HNH domains of SEQ ID NOs: 9-262.

In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 9-262. In some embodiments, the amino acid sequence of the RuvC domain is identical to the amino acid sequence of any of the RuvC domains of SEQ ID NOs: 9-262.

In some embodiments, the Cas9 protein comprises at D10A and/or a H840A mutation in the amino acid sequence provided in SEQ ID NO: 9, or a corresponding mutation in any of the amino acid sequences provided in SEQ ID NOs: 10-262. In some embodiments, the Cas9 protein comprises a D10X₁ and/or a H840X₂ mutation of the amino acid sequence provided in SEQ ID NO: 9, or a corresponding mutation in any of the amino acid sequences provided in SEQ ID NOs: 10-262, wherein X₁ is any amino acid except for D, and wherein X₂ is any amino acid except for H. In some embodiments, the Cas9 protein comprises an D10A mutation of the amino acid sequence provided in SEQ ID NO: 9, or a corresponding mutation in any of the amino acid sequences provided in SEQ ID NOs: 10-262. In some embodiments, the Cas9 protein comprises an H at amino acid residue 840 of the amino acid sequence provided in SEQ ID NO: 9, or a corresponding residue in any of the amino acid sequences provided in SEQ ID NOs: 10-262. In some embodiments, the Cas9 protein comprises an H840A mutation of the amino acid sequence provided in SEQ ID NO: 9, or a corresponding mutation in any of the amino acid sequences provided in SEQ ID NOs: 10-262. In some embodiments, the Cas9 protein comprises an D at amino acid residue 10 of the amino acid sequence provided in SEQ ID NO: 9, or a corresponding residue in any of the amino acid sequences provided in SEQ ID NOS: 10-262.

Some aspects of this disclosure provide fusion proteins comprising a Cas9 protein as provided herein that is fused to a second protein, thus forming a fusion protein. In some embodiments, the second protein is fused to the N-terminus of the Cas9 protein. In some embodiments, the second protein is fused to the C-terminus of the Cas9 protein. In some embodiments, the Cas9 domain and the effector domain are fused via a linker. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker comprises a chemical group or molecule linking two molecules or moieties, e.g., two domains of a fusion protein, such as, for example, a nuclease-inactive Cas9 domain and a effector domain (e.g., a deaminase domain). In some embodiments, the linker comprises one or more amino acid residues. For example, the linker may comprise at least 1, 2, 3, 4, 5, 6, 7, 8, 9, 10, 11, 12, 13, 14, 15, 16, 17, 18, 19, 20, 21, 25, 30, 35, 40, 45, 50, or more amino acid residues. In some embodiments, the linker is 3, 9, 16, or 21 amino acids in length. In some embodiments, the linker comprises a (GGGGS)ₙ(SEQ ID NO: 5), a (G)ₙ (SEQ ID NO: 5087), an (EAAAK)ₙ(SEQ ID NO: 6), a (GGS)ₙ(SEQ ID NO: 5088), an SGSETPGTSESATPES (SEQ ID NO: 7) (also referred to as XTEN), or an (XP)ₙ (SEQ ID NO: 5089) motif, or a combination of any of these, wherein n is independently an integer between 1 and 30. In some embodiments, wherein the linker comprises a (GGS)₃ (SEQ ID NO: 5088) motif or a SGSETPGTSESATPES (SEQ ID NO: 7) (XTEN) motif.

Some aspects of this disclosure provide fusion proteins comprising a Cas9 protein as provided herein that is fused to a second protein, thus forming a fusion protein. In some embodiments, the second protein is fused to the N-terminus of the Cas9 protein. In some embodiments, the second protein is fused to the C-terminus of the Cas9 protein. In some embodiments, the Cas9 domain and the effector domain are fused via a nuclear localization sequence (NLS), for example a NLS comprising the amino acid sequence PKKKRKV (SEQ ID NO: 299), MDSLLMNRRKFLYQFKNVRWAKGRRETYLC (SEQ ID NO: 300), or SPKKKRKVEAS (SEQ ID NO: 284). In some embodiments, a NLS may be combined with any of the linkers listed above.

In some embodiments, the effector domain comprises an enzymatic domain. In some embodiments, the effector domain comprises a nuclease, a nickase, a recombinase, a deaminase, a methyltransferase, a methylase, an acetylase, an acetyltransferase, a transcriptional activator, or a transcriptional repressor domain, which may have nuclease activity, nickase activity, recombinase activity, deaminase activity, methyltransferase activity, methylase activity, acetylase activity, acetyltransferase activity transcriptional activation activity or transcriptional repression activity, respectively. In some embodiments, the effector domain is a effector domain. In some embodiments, the effector domain is a deaminase domain. In some embodiments, the deaminase is a cytosine deaminase or a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the effector domain is at least 80%, at least 85%, at least 90%, at least 92%, at least 95%, at least 96%, at least 97%, at least 98%, at least 99%, or at least 99.5% identical to the deaminase domain of any one of SEQ ID NOs: 263-281. In some embodiments, the deaminase is a cytidine deaminase. In some embodiments, the deaminase is an apolipoprotein B mRNA-editing complex (APOBEC) family deaminase. In some embodiments, the deaminase is an APOBEC1 family deaminase. In some embodiments, the deaminase is an activation-induced cytidine deaminase (AID). In some embodiments, the deaminase is an ACF1/ASE deaminase. In some embodiments, the deaminase is an adenosine deaminase. In some embodiments, the deaminase is an ADAT family deaminase.

Some aspects of this disclosure provide fusion proteins comprising a Cas9 protein fused to a effector domain, e.g., a deaminase, and a uracil glycosylase inhibitor (UGI). Some aspects of this disclosure are based on the recognition that such fusion proteins may exhibit an increased nucleic acid editing efficiency as compared to fusion proteins not comprising an UGI domain. Domains such as the deaminase domains and UGI domains have been described and are within the scope of this disclosure. For example domains such as deaminase domains and UGI domains have been described in Provisional Application Nos. 62/245,828, filed Oct. 23, 2015, 62/279,346 filed Jan. 15, 2016, 62/311,763 filed Mar. 22, 2016, 62/322,178 filed Apr. 13, 2016, 62/357,352 filed Jun. 30, 2016, 62/370,700 filed Aug. 3, 2016, 62/398,490 filed Sep. 22, 2016, and 62/408,686 filed Oct. 14, 2016; the entire contents of each is incorporated by reference herein. It should be appreciated that the deaminase domains and UGI domains described in the foregoing references are within the scope of this disclosure and may be fused with any of the Cas9 proteins provided herein.

In some embodiments, the effector domain of the fusion protein is a nuclease domain. In some embodiments, the nuclease domain is a FokI DNA cleavage domain. In some embodiments, the fusion protein dimerizes. In certain embodiments, the dimer of the fusion protein is active. For example, two Fok1 DNA cleavage domains may dimerize to cleave a nucleic acid.

In some embodiments, the Cas9 protein is fused to a second Cas9 protein. In some embodiments, the second Cas9 protein is the Cas9 protein of any one of claims 1-345. In some embodiments, the second Cas9 protein is fused to the N-terminus of the fusion protein. In some embodiments, the second Cas9 protein is fused to the C-terminus of the fusion protein. In some embodiments, the Cas9 protein and the second Cas9 protein are fused via a second linker. In some embodiments, the second linker comprises a (GGGGS), (SEQ ID NO: 5), a (G), (SEQ ID NO: 5087), an (EAAAK), (SEQ ID NO: 6), a (GGS), (SEQ ID NO: 5088), an SGSETPGTSESATPES (SEQ ID NO: 7), or an (XP), (SEQ ID NO: 5089) motif, or a combination of any of these, wherein n is independently an integer between 1 and 30. In some embodiments, the second linker comprises a (GGS) 3 (SEQ ID NO: 5088) motif.

Some aspects of this disclosure provide complexes comprising a Cas9 protein, or a Cas9 fusion protein as provided herein, and a guide RNA bound to the Cas9 protein, or the Cas9 fusion protein.

In some embodiments, the target sequence is a DNA sequence. In some embodiments, the target sequence is a sequence in the genome of a mammal. In some embodiments, the target sequence is a sequence in the genome of a human. In some embodiments, the 3′-end of the target sequence is not immediately adjacent to the canonical PAM sequence (5′-NGG-3′).

Some aspects of this disclosure provide methods of using the Cas9 proteins, fusion proteins, or complexes provided herein. For example, some aspects of this disclosure provide methods comprising contacting a DNA molecule (a) with a Cas9 protein or a fusion protein as provided herein and a guide RNA, wherein the guide RNA is about 15-100 nucleotides long and comprises a sequence of at least 10 contiguous nucleotides that is complementary to a target sequence; or (b) with a Cas9 protein, a Cas9 fusion protein, or a Cas9 protein or fusion protein complex with a gRNA as provided herein. In some embodiments, the 3′-end of the target sequence is not immediately adjacent to the canonical PAM sequence (5′-NGG-3′). In some embodiments, the 3′-end of the target sequence is immediately adjacent to an AGC, GAG, TTT, GTG, or CAA sequence. In some embodiments, the target DNA sequence comprises a sequence associated with a disease or disorder. In some embodiments, the target DNA sequence comprises a point mutation associated with a disease or disorder. In some embodiments, the activity of the Cas9 protein, the Cas9 fusion protein, or the complex results in correction of the point mutation. In some embodiments, the step of contacting is performed in vivo in a subject.

Some aspects of this disclosure provide kits comprising a nucleic acid construct, comprising (a) a nucleotide sequence encoding a Cas9 protein or a Cas9 fusion protein as provided herein; and (b) a heterologous promoter that drives expression of the sequence of (a). In some embodiments, the kit further comprises an expression construct encoding a guide RNA backbone, wherein the construct comprises a cloning site positioned to allow the cloning of a nucleic acid sequence identical or complementary to a target sequence into the guide RNA backbone.

Some aspects of this disclosure provide polynucleotides encoding any of the Cas9 proteins, Cas9 fusion proteins, or guide RNA bound to the Cas9 protein or Cas9 fusion protein provided herein. Some aspects of this disclosure provide vectors comprising such polynucleotides. In some embodiments, the vector comprises a heterologous promoter driving expression of the polynucleotide.

Some aspects of this disclosure provide cells comprising any of the Cas9 proteins, fusion proteins, nucleic acid molecules, and/or a vectors as provided herein.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 shows the activity of wild-type Streptococcus pyogenes Cas9 on canonical PAM libraries and non-canonical PAM libraries.

FIG. 2 shows the activity of exemplary evolved Cas9 clones on a PAM library after directed evolution.

FIG. 3 shows a comparison of wild-type and evolved Cas9 in a mammalian GFP activation assay.

FIGS. 4A to 4B show the binding activity of Cas9 (pJH306) and evolved Cas9 proteins with an 5′-NGG-3′ PAM sequence using GFP as a readout. On 5′-NGG-3′ PAMs, many of the evolved Cas9 proteins showed increased Cas9 binding activity relative to wild-type Cas9 based on an increase in GFP fluorescence signal. FIG. 4A is a graph representing Cas9 binding activity as a function of % cells above the background fluorescence. FIG. 4B is a graph showing Cas9 binding activity as a function of mean fluorescence. Cas9 proteins used in these experiments were dCas9 proteins fused to a VPR transcriptional activator.

FIGS. 5A to 5B show the binding activity of wild type dCas9-VPR (pJH306) and evolved dCas9-VPR proteins with an NNN PAM sequence using GFP as a readout. On a library of NNN PAMs, many of the evolved Cas9 proteins showed increased Cas9 binding activity relative to wild-type Cas9 based on an increase in GFP fluorescence signal. FIG. 5A is a graph representing Cas9 binding activity as a function of % cells above background fluorescence. FIG. 5B is a graph showing Cas9 binding activity as a function of mean fluorescence. Cas9 proteins used in these experiments were dCas9 proteins fused to VPR.

FIG. 6 shows dCas9-VPR on all 64 PAM sequences as demonstrated by mean fluorescence on transfected cells gated by iRFP fluorescence. WT dCas9-VPR is pJH306.

FIG. 7 shows in vitro cutting assay. On the gel, WT is wild-type Cas9 (SEQ ID NO: 9), and 1 is Cas9 (SEQ ID NO: 9) with the E1219V mutation.

FIG. 8 shows a Cas9 fusion protein that can be used to modulate PAM specificity. One possible configuration for a linked Cas9-dCas9 system that could be used for increasing Cas9 targeting to non-canonical PAMs is shown. dCas9 binding to 5′-NGG-3′ or another PAM could localize Cas9 to an area close to target 2. This localization could help Cas9 cut a previously inaccessible PAM.

FIGS. 9A to 9B show dCas9-VPR binding activity on the NNNNN PAM Library. FIG. 9A is a graph representing dCas9-VPR binding activity on the NNNNN PAM library as a function of the % cells above background fluorescence. FIG. 9B is a graph representing dCas9-VPR binding activity on the NNNNN PAM library as a function of mean fluorescence.

FIG. 10 shows Cas9 cutting activity using % of cells with GFP loss as a readout. Cas9 proteins were tested with two sgRNAs, which either targeted the canonical 5′-NGG-3′ PAM or a GAT PAM within a GFP gene.

FIG. 11 illustrates double-stranded DNA substrate bound by Cas9:DNA editing enzyme:sgRNA complexes. The DNA editing enzyme may be, without limitation, a nuclease, nickase, recombinase, deaminase, methyltransferase, methylase, acetylase, or acetyltransferase.

FIG. 12 shows the results of the PAM depletion assay. pJH760 was tested in the PAM depletion assay on four new targets: re2, VEGF, CLTA, and CCR5D.

FIG. 13 shows GFP cutting in mammalian cells.

FIG. 14 shows the results of a PAM depletion assay to test pJH760 (xCas9 v1.0) on the re2 target.

FIG. 15 shows the results of a PAM depletion assay to test pJH760 (xCas9 v1.0) on the VEGF target.

FIG. 16 shows the results of a PAM depletion assay to test pJH760 (xCas9 v1.0) on the CLTA target.

FIG. 17 shows the results of a PAM depletion assay to test pJH760 (xCas9 v1.0) on the CCR5D target.

FIG. 18 shows the results of a PAM depletion assay to test four xCas9v3 mutants on the re2 target.

FIG. 19 shows the results of a PAM depletion assay to test four xCas9v3 mutants on the VEGF target.

FIG. 20 shows the results of a PAM depletion assay to test four xCas9v3 mutants on the CLTA target.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

The term “Cas9” or “Cas9 nuclease” refers to an RNA-guided nuclease comprising a Cas9 protein, or a fragment thereof (e.g., a protein comprising an active or inactive DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which are hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roc B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain.

A nuclease-inactivated Cas9 protein may interchangeably be referred to as a “dCas9” protein (for nuclease-“dead” Cas9). Methods for generating a Cas9 having an inactive DNA cleavage domain are known (See, e.g., Jinek et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821 (2012); Qi et al., Cell. 28; 152 (5): 1173-83 (2013).

In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type Cas9. In some embodiments, wild type Cas9 corresponds to Cas9 from Streptococcus pyogenes (NCBI Reference Sequence: NC_017053.1, SEQ ID NO:1 (nucleotide); SEQ ID NO:2 (amino acid)).

(SEQ ID NO: 1)
ATGGATAAGAAATACTCAATAGGCTTAGATATCGGCACAAATAGCGTCGGATGGGCGGTGATCACTGATGATTATAA
GGTTCCGTCTAAAAAGTTCAAGGTTCTGGGAAATACAGACCGCCACAGTATCAAAAAAAATCTTATAGGGGCTCTTT
TATTTGGCAGTGGAGAGACAGCGGAAGCGACTCGTCTCAAACGGACAGCTCGTAGAAGGTATACACGTCGGAAGAAT
CGTATTTGTTATCTACAGGAGATTTTTTCAAATGAGATGGCGAAAGTAGATGATAGTTTCTTTCATCGACTTGAAGA
GTCTTTTTTGGTGGAAGAAGACAAGAAGCATGAACGTCATCCTATTTTTGGAAATATAGTAGATGAAGTTGCTTATC
ATGAGAAATATCCAACTATCTATCATCTGCGAAAAAAATTGGCAGATTCTACTGATAAAGCGGATTTGCGCTTAATC
TATTTGGCCTTAGCGCATATGATTAAGTTTCGTGGTCATTTTTTGATTGAGGGAGATTTAAATCCTGATAATAGTGA
TGTGGACAAACTATTTATCCAGTTGGTACAAATCTACAATCAATTATTTGAAGAAAACCCTATTAACGCAAGTAGAG
TAGATGCTAAAGCGATTCTTTCTGCACGATTGAGTAAATCAAGACGATTAGAAAATCTCATTGCTCAGCTCCCCGGT
GAGAAGAGAAATGGCTTGTTTGGGAATCTCATTGCTTTGTCATTGGGATTGACCCCTAATTTTAAATCAAATTTTGA
TTTGGCAGAAGATGCTAAATTACAGCTTTCAAAAGATACTTACGATGATGATTTAGATAATTTATTGGCGCAAATTG
GAGATCAATATGCTGATTTGTTTTTGGCAGCTAAGAATTTATCAGATGCTATTTTACTTTCAGATATCCTAAGAGTA
AATAGTGAAATAACTAAGGCTCCCCTATCAGCTTCAATGATTAAGCGCTACGATGAACATCATCAAGACTTGACTCT
TTTAAAAGCTTTAGTTCGACAACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAATTTTAGAAAAAATGGATGGT
ACTGAGGAATTATTGGTGAAACTAAATCGTGAAGATTTGCTGCGCAAGCAACGGACCTTTGACAACGGCTCTATTCC
CCATCAAATTCACTTGGGTGAGCTGCATGCTATTTTGAGAAGACAAGAAGACTTTTATCCATTTTTAAAAGACAATC
GTGAGAAGATTGAAAAAATCTTGACTTTTCGAATTCCTTATTATGTTGGTCCATTGGCGCGTGGCAATAGTCGTTTT
GCATGGATGACTCGGAAGTCTGAAGAAACAATTACCCCATGGAATTTTGAAGAAGTTGTCGATAAAGGTGCTTCAGC
TCAATCATTTATTGAACGCATGACAAACTTTGATAAAAATCTTCCAAATGAAAAAGTACTACCAAAACATAGTTTGC
TTTATGAGTATTTTACGGTTTATAACGAATTGACAAAGGTCAAATATGTTACTGAGGGAATGCGAAAACCAGCATTT
CTTTCAGGTGAACAGAAGAAAGCCATTGTTGATTTACTCTTCAAAACAAATCGAAAAGTAACCGTTAAGCAATTAAA
AGAAGATTATTTCAAAAAAATAGAATGTTTTGATAGTGTTGAAATTTCAGGAGTTGAAGATAGATTTAATGCTTCAT
TAGGCGCCTACCATGATTTGCTAAAAATTATTAAAGATAAAGATTTTTTGGATAATGAAGAAAATGAAGATATCTTA
GAGGATATTGTTTTAACATTGACCTTATTTGAAGATAGGGGGATGATTGAGGAAAGACTTAAAACATATGCTCACCT
CTTTGATGATAAGGTGATGAAACAGCTTAAACGTCGCCGTTATACTGGTTGGGGACGTTTGTCTCGAAAATTGATTA
ATGGTATTAGGGATAAGCAATCTGGCAAAACAATATTAGATTTTTTGAAATCAGATGGTTTTGCCAATCGCAATTTT
ATGCAGCTGATCCATGATGATAGTTTGACATTTAAAGAAGATATTCAAAAAGCACAGGTGTCTGGACAAGGCCATAG
TTTACATGAACAGATTGCTAACTTAGCTGGCAGTCCTGCTATTAAAAAAGGTATTTTACAGACTGTAAAAATTGTTG
ATGAACTGGTCAAAGTAATGGGGCATAAGCCAGAAAATATCGTTATTGAAATGGCACGTGAAAATCAGACAACTCAA
AAGGGCCAGAAAAATTCGCGAGAGCGTATGAAACGAATCGAAGAAGGTATCAAAGAATTAGGAAGTCAGATTCTTAA
AGAGCATCCTGTTGAAAATACTCAATTGCAAAATGAAAAGCTCTATCTCTATTATCTACAAAATGGAAGAGACATGT
ATGTGGACCAAGAATTAGATATTAATCGTTTAAGTGATTATGATGTCGATCACATTGTTCCACAAAGTTTCATTAAA
GACGATTCAATAGACAATAAGGTACTAACGCGTTCTGATAAAAATCGTGGTAAATCGGATAACGTTCCAAGTGAAGA
AGTAGTCAAAAAGATGAAAAACTATTGGAGACAACTTCTAAACGCCAAGTTAATCACTCAACGTAAGTTTGATAATT
TAACGAAAGCTGAACGTGGAGGTTTGAGTGAACTTGATAAAGCTGGTTTTATCAAACGCCAATTGGTTGAAACTCGC
CAAATCACTAAGCATGTGGCACAAATTTTGGATAGTCGCATGAATACTAAATACGATGAAAATGATAAACTTATTCG
AGAGGTTAAAGTGATTACCTTAAAATCTAAATTAGTTTCTGACTTCCGAAAAGATTTCCAATTCTATAAAGTACGTG
AGATTAACAATTACCATCATGCCCATGATGCGTATCTAAATGCCGTCGTTGGAACTGCTTTGATTAAGAAATATCCA
AAACTTGAATCGGAGTTTGTCTATGGTGATTATAAAGTTTATGATGTTCGTAAAATGATTGCTAAGTCTGAGCAAGA
AATAGGCAAAGCAACCGCAAAATATTTCTTTTACTCTAATATCATGAACTTCTTCAAAACAGAAATTACACTTGCAA
ATGGAGAGATTCGCAAACGCCCTCTAATCGAAACTAATGGGGAAACTGGAGAAATTGTCTGGGATAAAGGGCGAGAT
TTTGCCACAGTGCGCAAAGTATTGTCCATGCCCCAAGTCAATATTGTCAAGAAAACAGAAGTACAGACAGGCGGATT
CTCCAAGGAGTCAATTTTACCAAAAAGAAATTCGGACAAGCTTATTGCTCGTAAAAAAGACTGGGATCCAAAAAAAT
ATGGTGGTTTTGATAGTCCAACGGTAGCTTATTCAGTCCTAGTGGTTGCTAAGGTGGAAAAAGGGAAATCGAAGAAG
TTAAAATCCGTTAAAGAGTTACTAGGGATCACAATTATGGAAAGAAGTTCCTTTGAAAAAAATCCGATTGACTTTTT
AGAAGCTAAAGGATATAAGGAAGTTAAAAAAGACTTAATCATTAAACTACCTAAATATAGTCTTTTTGAGTTAGAAA
ACGGTCGTAAACGGATGCTGGCTAGTGCCGGAGAATTACAAAAAGGAAATGAGCTGGCTCTGCCAAGCAAATATGTG
AATTTTTTATATTTAGCTAGTCATTATGAAAAGTTGAAGGGTAGTCCAGAAGATAACGAACAAAAACAATTGTTTGT
GGAGCAGCATAAGCATTATTTAGATGAGATTATTGAGCAAATCAGTGAATTTTCTAAGCGTGTTATTTTAGCAGATG
CCAATTTAGATAAAGTTCTTAGTGCATATAACAAACATAGAGACAAACCAATACGTGAACAAGCAGAAAATATTATT
CATTTATTTACGTTGACGAATCTTGGAGCTCCCGCTGCTTTTAAATATTTTGATACAACAATTGATCGTAAACGATA
TACGTCTACAAAAGAAGTTTTAGATGCCACTCTTATCCATCAATCCATCACTGGTCTTTATGAAACACGCATTGATT
TGAGTCAGCTAGGAGGTGACTGA
(SEQ ID NO: 2)
MDKKYSIGLDIGTNSVGWAVITDDYKVPSKKFKVLGNTDRHSIKKNLIGALLFGSGETAEATRLKRTARRRYTRRKN
RICYLQEIFSNEMAKVDDSFFHRLEESFLVEEDKKHERHPIFGNIVDEVAYHEKYPTIYHLRKKLADSTDKADLRLI
YLALAHMIKFRGHFLIEGDLNPDNSDVDKLFIQLVQIYNQLFEENPINASRVDAKAILSARLSKSRRLENLIAQLPG
EKRNGLFGNLIALSLGLTPNFKSNFDLAEDAKLQLSKDTYDDDLDNLLAQIGDQYADLFLAAKNLSDAILLSDILRV
NSEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNASLGAYHDLLKIIKDKDFLDNEENEDIL
EDIVLTLTLFEDRGMIEERLKTYAHLFDDKVMKQLKRRRYTGWGRLSRKLINGIRDKQSGKTILDFLKSDGFANRNF
MQLIHDDSLTFKEDIQKAQVSGQGHSLHEQIANLAGSPAIKKGILOTVKIVDELVKVMGHKPENIVIEMARENQTTQ
KGQKNSRERMKRIEEGIKELGSQILKEHPVENTQLQNEKLYLYYLQNGRDMYVDQELDINRLSDYDVDHIVPQSFIK
DDSIDNKVLTRSDKNRGKSDNVPSEEVVKKMKNYWRQLLNAKLITQRKFDNLTKAERGGLSELDKAGFIKROLVETR
QITKHVAQILDSRMNTKYDENDKLIREVKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITLANGEIRKRPLIETNGETGEIVWDKGRD
FATVRKVLSMPQVNIVKKTEVQTGGFSKESILPKRNSDKLIARKKDWDPKKYGGEDSPTVAYSVLVVAKVEKGKSKK
LKSVKELLGITIMERSSFEKNPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVLSAYNKHRDKPIREQAENII
HLFTLTNLGAPAAFKYFDTTIDRKRYTSTKEVLDATLIHQSITGLYETRIDLSQLGGD
(single underline: HNH domain; double underline: RuvC domain)

In some embodiments, wild type Cas9 corresponds to, or comprises SEQ ID NO:3 (nucleotide) and/or SEQ ID NO: 4 (amino acid):

(SEQ ID NO: 3)
ATGGATAAAAAGTATTCTATTGGTTTAGACATCGGCACTAATTCCGTTGGATGGGCTGTCATAACCGATGAATACAA
AGTACCTTCAAAGAAATTTAAGGTGTTGGGGAACACAGACCGTCATTCGATTAAAAAGAATCTTATCGGTGCCCTCC
TATTCGATAGTGGCGAAACGGCAGAGGCGACTCGCCTGAAACGAACCGCTCGGAGAAGGTATACACGTCGCAAGAAC
CGAATATGTTACTTACAAGAAATTTTTAGCAATGAGATGGCCAAAGTTGACGATTCTTTCTTTCACCGTTTGGAAGA
GTCCTTCCTTGTCGAAGAGGACAAGAAACATGAACGGCACCCCATCTTTGGAAACATAGTAGATGAGGTGGCATATC
ATGAAAAGTACCCAACGATTTATCACCTCAGAAAAAAGCTAGTTGACTCAACTGATAAAGCGGACCTGAGGTTAATC
TACTTGGCTCTTGCCCATATGATAAAGTTCCGTGGGCACTTTCTCATTGAGGGTGATCTAAATCCGGACAACTCGGA
TGTCGACAAACTGTTCATCCAGTTAGTACAAACCTATAATCAGTTGTTTGAAGAGAACCCTATAAATGCAAGTGGCG
TGGATGCGAAGGCTATTCTTAGCGCCCGCCTCTCTAAATCCCGACGGCTAGAAAACCTGATCGCACAATTACCCGGA
GAGAAGAAAAATGGGTTGTTCGGTAACCTTATAGCGCTCTCACTAGGCCTGACACCAAATTTTAAGTCGAACTTCGA
CTTAGCTGAAGATGCCAAATTGCAGCTTAGTAAGGACACGTACGATGACGATCTCGACAATCTACTGGCACAAATTG
GAGATCAGTATGCGGACTTATTTTTGGCTGCCAAAAACCTTAGCGATGCAATCCTCCTATCTGACATACTGAGAGTT
AATACTGAGATTACCAAGGCGCCGTTATCCGCTTCAATGATCAAAAGGTACGATGAACATCACCAAGACTTGACACT
TCTCAAGGCCCTAGTCCGTCAGCAACTGCCTGAGAAATATAAGGAAATATTCTTTGATCAGTCGAAAAACGGGTACG
CAGGTTATATTGACGGCGGAGCGAGTCAAGAGGAATTCTACAAGTTTATCAAACCCATATTAGAGAAGATGGATGGG
ACGGAAGAGTTGCTTGTAAAACTCAATCGCGAAGATCTACTGCGAAAGCAGCGGACTTTCGACAACGGTAGCATTCC
ACATCAAATCCACTTAGGCGAATTGCATGCTATACTTAGAAGGCAGGAGGATTTTTATCCGTTCCTCAAAGACAATC
GTGAAAAGATTGAGAAAATCCTAACCTTTCGCATACCTTACTATGTGGGACCCCTGGCCCGAGGGAACTCTCGGTTC
GCATGGATGACAAGAAAGTCCGAAGAAACGATTACTCCATGGAATTTTGAGGAAGTTGTCGATAAAGGTGCGTCAGC
TCAATCGTTCATCGAGAGGATGACCAACTTTGACAAGAATTTACCGAACGAAAAAGTATTGCCTAAGCACAGTTTAC
TTTACGAGTATTTCACAGTGTACAATGAACTCACGAAAGTTAAGTATGTCACTGAGGGCATGCGTAAACCCGCCTTT
CTAAGCGGAGAACAGAAGAAAGCAATAGTAGATCTGTTATTCAAGACCAACCGCAAAGTGACAGTTAAGCAATTGAA
AGAGGACTACTTTAAGAAAATTGAATGCTTCGATTCTGTCGAGATCTCCGGGGTAGAAGATCGATTTAATGCGTCAC
TTGGTACGTATCATGACCTCCTAAAGATAATTAAAGATAAGGACTTCCTGGATAACGAAGAGAATGAAGATATCTTA
GAAGATATAGTGTTGACTCTTACCCTCTTTGAAGATCGGGAAATGATTGAGGAAAGACTAAAAACATACGCTCACCT
GTTCGACGATAAGGTTATGAAACAGTTAAAGAGGCGTCGCTATACGGGCTGGGGACGATTGTCGCGGAAACTTATCA
ACGGGATAAGAGACAAGCAAAGTGGTAAAACTATTCTCGATTTTCTAAAGAGCGACGGCTTCGCCAATAGGAACTTT
ATGCAGCTGATCCATGATGACTCTTTAACCTTCAAAGAGGATATACAAAAGGCACAGGTTTCCGGACAAGGGGACTC
ATTGCACGAACATATTGCGAATCTTGCTGGTTCGCCAGCCATCAAAAAGGGCATACTCCAGACAGTCAAAGTAGTGG
ATGAGCTAGTTAAGGTCATGGGACGTCACAAACCGGAAAACATTGTAATCGAGATGGCACGCGAAAATCAAACGACT
CAGAAGGGGCAAAAAAACAGTCGAGAGCGGATGAAGAGAATAGAAGAGGGTATTAAAGAACTGGGCAGCCAGATCTT
AAAGGAGCATCCTGTGGAAAATACCCAATTGCAGAACGAGAAACTTTACCTCTATTACCTACAAAATGGAAGGGACA
TGTATGTTGATCAGGAACTGGACATAAACCGTTTATCTGATTACGACGTCGATCACATTGTACCCCAATCCTTTTTG
AAGGACGATTCAATCGACAATAAAGTGCTTACACGCTCGGATAAGAACCGAGGGAAAAGTGACAATGTTCCAAGCGA
GGAAGTCGTAAAGAAAATGAAGAACTATTGGCGGCAGCTCCTAAATGCGAAACTGATAACGCAAAGAAAGTTCGATA
ACTTAACTAAAGCTGAGAGGGGTGGCTTGTCTGAACTTGACAAGGCCGGATTTATTAAACGTCAGCTCGTGGAAACC
CGCCAAATCACAAAGCATGTTGCACAGATACTAGATTCCCGAATGAATACGAAATACGACGAGAACGATAAGCTGAT
TCGGGAAGTCAAAGTAATCACTTTAAAGTCAAAATTGGTGTCGGACTTCAGAAAGGATTTTCAATTCTATAAAGTTA
GGGAGATAAATAACTACCACCATGCGCACGACGCTTATCITAATGCCGTCGTAGGGACCGCACTCATTAAGAAATAC
CCGAAGCTAGAAAGTGAGTTTGTGTATGGTGATTACAAAGTTTATGACGTCCGTAAGATGATCGCGAAAAGCGAACA
GGAGATAGGCAAGGCTACAGCCAAATACTTCTTTTATTCTAACATTATGAATTTCTTTAAGACGGAAATCACTCTGG
CAAACGGAGAGATACGCAAACGACCTTTAATTGAAACCAATGGGGAGACAGGTGAAATCGTATGGGATAAGGGCCGG
GACTTCGCGACGGTGAGAAAAGTTTTGTCCATGCCCCAAGTCAACATAGTAAAGAAAACTGAGGTGCAGACCGGAGG
GTTTTCAAAGGAATCGATTCTTCCAAAAAGGAATAGTGATAAGCTCATCGCTCGTAAAAAGGACTGGGACCCGAAAA
AGTACGGTGGCTTCGATAGCCCTACAGTTGCCTATTCTGTCCTAGTAGTGGCAAAAGTTGAGAAGGGAAAATCCAAG
AAACTGAAGTCAGTCAAAGAATTATTGGGGATAACGATTATGGAGCGCTCGTCTTTTGAAAAGAACCCCATCGACTT
CCTTGAGGCGAAAGGTTACAAGGAAGTAAAAAAGGATCTCATAATTAAACTACCAAAGTATAGTCTGTTTGAGTTAG
AAAATGGCCGAAAACGGATGTTGGCTAGCGCCGGAGAGCTTCAAAAGGGGAACGAACTCGCACTACCGTCTAAATAC
GTGAATTTCCTGTATTTAGCGTCCCATTACGAGAAGTTGAAAGGTTCACCTGAAGATAACGAACAGAAGCAACTTTT
TGTTGAGCAGCACAAACATTATCTCGACGAAATCATAGAGCAAATTTCGGAATTCAGTAAGAGAGTCATCCTAGCTG
ATGCCAATCTGGACAAAGTATTAAGCGCATACAACAAGCACAGGGATAAACCCATACGTGAGCAGGCGGAAAATATT
ATCCATTTGTTTACTCTTACCAACCTCGGCGCTCCAGCCGCATTCAAGTATTTTGACACAACGATAGATCGCAAACG
ATACACTTCTACCAAGGAGGTGCTAGACGCGACACTGATTCACCAATCCATCACGGGATTATATGAAACTCGGATAG
ATTTGTCACAGCTTGGGGGTGACGGATCCCCCAAGAAGAAGAGGAAAGTCTCGAGCGACTACAAAGACCATGACGGT
GATTATAAAGATCATGACATCGATTACAAGGATGACGATGACAAGGCTGCAGGA
(SEQ ID NO: 4)
MDKKYSIGLAIGTNSVGWAVITDEYKVPSKKFKVLGNTDRHSIKKNLIGALLFDSGETAEATRLKRTARRRYTRRKN
RICYLQEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRENASLGTYHDLLKIIKDKDELDNEENEDIL
EDIVLTLTLFEDREMIEERLKTYAHLEDDKVMKQLKRRRYTGWGRLSRKLINGIRDKQSGKTILDELKSDGFANRNE
MQLIHDDSLTFKEDIQKAQVSGQGDSLHEHIANLAGSPAIKKGILOTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDERKDFOFYKVREINNYHHAHDAYLNAVVGTALIKKY
PKLESEFVYGDYKVYDVRKMIAKSEQEIGKATAKYFFYSNIMNFFKTEITLANGEIRKRPLIETNGETGEIVWDKGR
DFATVRKVLSMPOVNIVKKTEVOTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(single underline: HNH domain; double underline: RuvC domain)

In some embodiments, wild type Cas9 corresponds to Cas9 from Streptococcus pyogenes (NCBI Reference Sequence: NC_002737.2. SEQ ID NO: 282 (nucleotide); and Uniport Reference Sequence: Q99ZW2. SEQ ID NO: 9 (amino acid).

(SEQ ID NO: 282)
ATGGATAAGAAATACTCAATAGGCTTAGATATCGGCACAAATAGCGTCGGATGGGCGGTGATCACTGATGAATATAA
GGTTCCGTCTAAAAAGTTCAAGGTTCTGGGAAATACAGACCGCCACAGTATCAAAAAAAATCTTATAGGGGCTCTTT
TATTTGACAGTGGAGAGACAGCGGAAGCGACTCGTCTCAAACGGACAGCTCGTAGAAGGTATACACGTCGGAAGAAT
CGTATTTGTTATCTACAGGAGATTTTTTCAAATGAGATGGCGAAAGTAGATGATAGTTTCTTTCATCGACTTGAAGA
GTCTTTTTTGGTGGAAGAAGACAAGAAGCATGAACGTCATCCTATTTTTGGAAATATAGTAGATGAAGTTGCTTATC
ATGAGAAATATCCAACTATCTATCATCTGCGAAAAAAATTGGTAGATTCTACTGATAAAGCGGATTTGCGCTTAATC
TATTTGGCCTTAGCGCATATGATTAAGTTTCGTGGTCATTTTTTGATTGAGGGAGATTTAAATCCTGATAATAGTGA
TGTGGACAAACTATTTATCCAGTTGGTACAAACCTACAATCAATTATTTGAAGAAAACCCTATTAACGCAAGTGGAG
TAGATGCTAAAGCGATTCTTTCTGCACGATTGAGTAAATCAAGACGATTAGAAAATCTCATTGCTCAGCTCCCCGGT
GAGAAGAAAAATGGCTTATTTGGGAATCTCATTGCTTTGTCATTGGGTTTGACCCCTAATTTTAAATCAAATTTTGA
TTTGGCAGAAGATGCTAAATTACAGCTTTCAAAAGATACTTACGATGATGATTTAGATAATTTATTGGCGCAAATTG
GAGATCAATATGCTGATTTGTTTTTGGCAGCTAAGAATTTATCAGATGCTATTTTACTTTCAGATATCCTAAGAGTA
AATACTGAAATAACTAAGGCTCCCCTATCAGCTTCAATGATTAAACGCTACGATGAACATCATCAAGACTTGACTCT
TTTAAAAGCTTTAGTTCGACAACAACTTCCAGAAAAGTATAAAGAAATCTTTTTTGATCAATCAAAAAACGGATATG
CAGGTTATATTGATGGGGGAGCTAGCCAAGAAGAATTTTATAAATTTATCAAACCAATTTTAGAAAAAATGGATGGT
ACTGAGGAATTATTGGTGAAACTAAATCGTGAAGATTTGCTGCGCAAGCAACGGACCTTTGACAACGGCTCTATTCC
CCATCAAATTCACTTGGGTGAGCTGCATGCTATTTTGAGAAGACAAGAAGACTTTTATCCATTTTTAAAAGACAATC
GTGAGAAGATTGAAAAAATCTTGACTTTTCGAATTCCTTATTATGTTGGTCCATTGGCGCGTGGCAATAGTCGTTTT
GCATGGATGACTCGGAAGTCTGAAGAAACAATTACCCCATGGAATTTTGAAGAAGTTGTCGATAAAGGTGCTTCAGC
TCAATCATTTATTGAACGCATGACAAACTTTGATAAAAATCTTCCAAATGAAAAAGTACTACCAAAACATAGTTTGC
TTTATGAGTATTTTACGGTTTATAACGAATTGACAAAGGTCAAATATGTTACTGAAGGAATGCGAAAACCAGCATTT
CTTTCAGGTGAACAGAAGAAAGCCATTGTTGATTTACTCTTCAAAACAAATCGAAAAGTAACCGTTAAGCAATTAAA
AGAAGATTATTTCAAAAAAATAGAATGTTTTGATAGTGTTGAAATTTCAGGAGTTGAAGATAGATTTAATGCTTCAT
TAGGTACCTACCATGATTTGCTAAAAATTATTAAAGATAAAGATTTTTTGGATAATGAAGAAAATGAAGATATCTTA
GAGGATATTGTTTTAACATTGACCTTATTTGAAGATAGGGAGATGATTGAGGAAAGACTTAAAACATATGCTCACCT
CTTTGATGATAAGGTGATGAAACAGCTTAAACGTCGCCGTTATACTGGTTGGGGACGTTTGTCTCGAAAATTGATTA
ATGGTATTAGGGATAAGCAATCTGGCAAAACAATATTAGATTTTTTGAAATCAGATGGTTTTGCCAATCGCAATTTT
ATGCAGCTGATCCATGATGATAGTTTGACATTTAAAGAAGACATTCAAAAAGCACAAGTGTCTGGACAAGGCGATAG
TTTACATGAACATATTGCAAATTTAGCTGGTAGCCCTGCTATTAAAAAAGGTATTTTACAGACTGTAAAAGTTGTTG
ATGAATTGGTCAAAGTAATGGGGCGGCATAAGCCAGAAAATATCGTTATTGAAATGGCACGTGAAAATCAGACAACT
CAAAAGGGCCAGAAAAATTCGCGAGAGCGTATGAAACGAATCGAAGAAGGTATCAAAGAATTAGGAAGTCAGATTCT
TAAAGAGCATCCTGTTGAAAATACTCAATTGCAAAATGAAAAGCTCTATCTCTATTATCTCCAAAATGGAAGAGACA
TGTATGTGGACCAAGAATTAGATATTAATCGTTTAAGTGATTATGATGTCGATCACATTGTTCCACAAAGTTTCCTT
AAAGACGATTCAATAGACAATAAGGTCTTAACGCGTTCTGATAAAAATCGTGGTAAATCGGATAACGTTCCAAGTGA
AGAAGTAGTCAAAAAGATGAAAAACTATTGGAGACAACTTCTAAACGCCAAGTTAATCACTCAACGTAAGTTTGATA
ATTTAACGAAAGCTGAACGTGGAGGTTTGAGTGAACTTGATAAAGCTGGTTTTATCAAACGCCAATTGGTTGAAACT
CGCCAAATCACTAAGCATGTGGCACAAATTTTGGATAGTCGCATGAATACTAAATACGATGAAAATGATAAACTTAT
TCGAGAGGTTAAAGTGATTACCTTAAAATCTAAATTAGTTTCTGACTTCCGAAAAGATTTCCAATTCTATAAAGTAC
GTGAGATTAACAATTACCATCATGCCCATGATGCGTATCTAAATGCCGTCGTTGGAACTGCTTTGATTAAGAAATAT
CCAAAACTTGAATCGGAGTTTGTCTATGGTGATTATAAAGTTTATGATGTTCGTAAAATGATTGCTAAGTCTGAGCA
AGAAATAGGCAAAGCAACCGCAAAATATTTCTTTTACTCTAATATCATGAACTTCTTCAAAACAGAAATTACACTTG
CAAATGGAGAGATTCGCAAACGCCCTCTAATCGAAACTAATGGGGAAACTGGAGAAATTGTCTGGGATAAAGGGCGA
GATTTTGCCACAGTGCGCAAAGTATTGTCCATGCCCCAAGTCAATATTGTCAAGAAAACAGAAGTACAGACAGGCGG
ATTCTCCAAGGAGTCAATTTTACCAAAAAGAAATTCGGACAAGCTTATTGCTCGTAAAAAAGACTGGGATCCAAAAA
AATATGGTGGTTTTGATAGTCCAACGGTAGCTTATTCAGTCCTAGTGGTTGCTAAGGTGGAAAAAGGGAAATCGAAG
AAGTTAAAATCCGTTAAAGAGTTACTAGGGATCACAATTATGGAAAGAAGTTCCTTTGAAAAAAATCCGATTGACTT
TTTAGAAGCTAAAGGATATAAGGAAGTTAAAAAAGACTTAATCATTAAACTACCTAAATATAGTCTTTTTGAGTTAG
AAAACGGTCGTAAACGGATGCTGGCTAGTGCCGGAGAATTACAAAAAGGAAATGAGCTGGCTCTGCCAAGCAAATAT
GTGAATTTTTTATATTTAGCTAGTCATTATGAAAAGTTGAAGGGTAGTCCAGAAGATAACGAACAAAAACAATTGTT
TGTGGAGCAGCATAAGCATTATTTAGATGAGATTATTGAGCAAATCAGTGAATTTTCTAAGCGTGTTATTTTAGCAG
ATGCCAATTTAGATAAAGTTCTTAGTGCATATAACAAACATAGAGACAAACCAATACGTGAACAAGCAGAAAATATT
ATTCATTTATTTACGTTGACGAATCTTGGAGCTCCCGCTGCTTTTAAATATTTTGATACAACAATTGATCGTAAACG
ATATACGTCTACAAAAGAAGTTTTAGATGCCACTCTTATCCATCAATCCATCACTGGTCTTTATGAAACACGCATTG
ATTTGAGTCAGCTAGGAGGTGACTGA
(SEQ ID NO: 9)
MDKKYSIGLDIGTNSVGWAVITDEYKVPSKKFKVLGNTDRHSIKKNLIGALLEDSGETAEATRLKRTARRRYTRRKN
RICYLQEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRENASLGTYHDLLKIIKDKDELDNEENEDIL
EDIVLTLTLFEDREMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKFDNLTKAERGGLSELDKAGFIKRQLVET
ROITKHVAQILDSRMNTKYDENDKLIREVKVITLKSKLVSDERKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIETNGETGEIVWDKGR
DFATVRKVLSMPOVNIVKKTEVOTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(single underline: HNH domain; double underline: RuvC domain)

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1) or to a Cas9 from any of the organisms listed in Example 3.

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and/or H840A mutation.

dCas9 (D10A and H840A):
(SEQ ID NO: 8)
MDKKYSIGLA*IGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNI*VDEVAYHEKYPTIYH*LRKKLVDSTDKADL
RLIYLALAHMIKFRGHFLIEGDLNPDNSDVD*KLFIQLVQTYNQLFEENPINASGVDAKAILSARLSKSRRLENLIA
QLPGEKKNGLFGNLIALSLGLTPNFKSNFDLAEDA*KLQLSKDTYDDDLDNLLAQIGDQYADLFLAAK*NLSDAILL
SDILRVNTEITKAPLSASMIKRYDEHHQDLTLLKALVRQQLPEKYKEIFFDQSKNGYAGYIDGGASQEEFYKFIKPI
LEKMDGTEELLVKLNREDLLRKORTFDNGS*IPHQIHLGELHAILRRQEDFYPFLKDNREKIEKILTFRIPYYVGPL
ARGNSRFAWMTRKSEETITPWNFEE*VVDKGASAQSFIERMTNFDKNLPNEKVLPKHSLLYEYFTVYNELTKVKYVT
EGMRKPAFLSGE*QKKAIVDLLFKTNRKVTVKOLKEDYFKKIECFDSVEISGVEDRENASLGTYHDLLKIIKDKDEL
DNEENEDILEDIVLTLTLFEDREMIEERLKTYAHLFDDKVMKQLKRRRYTGWG*RLSRKLINGIRDKQSGKTILDEL

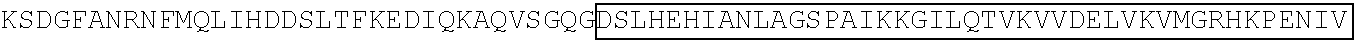

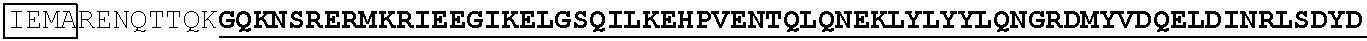

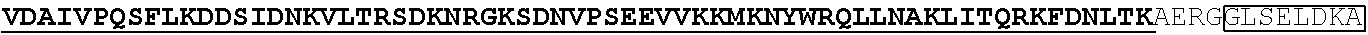

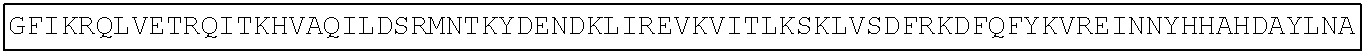

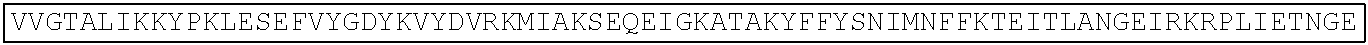

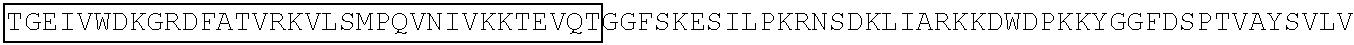

VAKVEKGKSKKLKSVKELLGITIMERSSFEKNPIDFLEAKGYKEVKKDLIIKLPKYSLFELENGRKRMLASAGELQK
GNELALPSKYVNFLYLASHYEKLKGSPEDNEQKOLFVEQHKHYLDEIIEQISEFSKRVILADANLDKVLSAYNKHRD
KPIREQAENIIHLFTLTNLGAPAAFKYFDTTIDRKRYTSTKEVLDATLIHQSITGLYETRIDLSQLGGD
(single underline: HNH domain; double underline: RuvC domain)

In some embodiments, Cas9 corresponds to, or comprises in part or in whole, a Cas9 amino acid sequence having one or more mutations that alter Cas9's nuclease activity. In some embodiments, Cas9 may be a Cas9 nickase, which is a version of Cas9 that generates a single-stranded DNA break at a specific location based on a co-expressed gRNA-defined target sequence, rather than a double-strand DNA break. For example, in some embodiments, a Cas9 domain comprises D10A mutation (e.g., SEQ ID NO: 301) and/or an H840A mutation (e.g., SEQ ID NO: 302). Exemplary Cas9 nickases are shown below. However, it should be appreciated that additional Cas9 nickases that generate a single-stranded DNA break of a DNA duplex would be apparent to the skilled artisan and are within the scope of this disclosure.

Cas9 D10A nickase:
(SEQ ID NO: 301)
MDKKYSIGLAIGTNSVGWAVITDEYKVPSKKFKVLGNTDRHSIKKNLIGALLFDSGETAEATRLKRTARRRYTRRKN
RICYLQEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRENASLGTYHDLLKIIKDKDELDNEENEDIL
EDIVLTLTLFEDREMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILOTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDHIVPQSFL
KDDSIDNKVLTRSDKNRGKSDNVPSEEVVKKMKNYWRQLLNAKLITQRKFDNLTKAERGGLSELDKAGFIKROLVET
RQITKHVAQILDSRMNTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIETNGETGEIVWDKGR
DFATVRKVLSMPOVNIVKKTEVOTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(single underline: HNH domain; double underline: RuvC domain)
Cas9 H840A nickase:
(SEQ ID NO: 302)
MDKKYSIGLDIGTNSVGWAVITDEYKVPSKKFKVLGNTDRHSIKKNLIGALLEDSGETAEATRLKRTARRRYTRRKN
RICYLQEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQEEFYKFIKPILEKMDG
TEELLVKLNREDLLRKQRTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNASLGTYHDLLKIIKDKDELDNEENEDIL
EDIVLTLTLFEDREMIEERLKTYAHLFDDKVMKQLKRRRYTGWGRLSRKLINGIRDKQSGKTILDFLKSDGFANRNE
MQLIHDDSLTFKEDIQKAQVSGQGDSLHEHIANLAGSPAIKKGILOTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDAIVPQSFL
KDDSIDNKVLTRSDKNRGKSDNVPSEEVVKKMKNYWRQLLNAKLITQRKFDNLTKAERGGLSELDKAGFIKRQLVET
RQITKHVAQILDSRMNTKYDENDKLIREVKVITLKSKLVSDERKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIETNGETGEIVWDKGR
DFATVRKVLSMPOVNIVKKTEVOTGGFSKESILPKRNSDKLIARKKDWDPKKYGGEDSPTVAYSVLVVAKVEKGKSK
KLKSVKELLGITIMERSSFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(single underline: HNH domain; double underline: RuvC domain)

In other embodiments, dCas9 variants having mutations other than D10A and H840A are provided, which, e.g., result in nuclease-inactivated Cas9 (dCas9). Such mutations, by way of example, include other amino acid substitutions at D10 and H820, or other substitutions within the nuclease domains of Cas9 (e.g., substitutions in the HNH nuclease subdomain and/or the RuvC1 subdomain). In some embodiments, variants or homologues of dCas9 (e.g., variants of SEQ ID NO: 9) are provided which are at least about 70% identical, at least about 80% identical, at least about 90% identical, at least about 95% identical, at least about 98% identical, at least about 99% identical, at least about 99.5% identical, or at least about 99.9% identical to SEQ ID NO: 9. In some embodiments, variants of dCas9 (e.g., variants of SEQ ID NO: 9) are provided having amino acid sequences which are shorter, or longer than SEQ ID NO: 9, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids, or more.

In some embodiments, Cas9 fusion proteins as provided herein comprise the full-length amino acid sequence of a Cas9 protein, e.g., one of the sequences provided above. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or a sgRNA, but does not comprise a functional nuclease domain, e.g., it comprises only a truncated version of a nuclease domain or no nuclease domain at all. Exemplary amino acid sequences of suitable Cas9 domains and Cas9 fragments are provided herein, and additional suitable sequences of Cas9 domains and Cas9 fragments will be apparent to those of skill in the art. In some embodiments, a Cas9 fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9 protein. In some embodiments, a Cas9 fragment comprises at least at least 100 amino acids in length. In some embodiments, the Cas9 fragment is at least 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, or at least 1600 amino acids of a corresponding wild type Cas9 protein. In some embodiments, the Cas9 fragment comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues of a corresponding wild type Cas9 protein.

Cas9. In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisI (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1).

The term “deaminase” or “deaminase domain,” as used herein, refers to a protein or enzyme that catalyzes a deamination reaction. In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytosine deaminase, catalyzing the hydrolytic deamination of cytosine to uracil. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism, that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a fusion protein provided herein, e.g., of a fusion protein comprising a nuclease-inactive Cas9 domain and a effector domain (e.g., a deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nuclease, a deaminase, a recombinase, a hybrid protein, a protein dimer, a complex of a protein (or protein dimer) and a polynucleotide, or a polynucleotide, may vary depending on various factors such as, for example, on the desired biological response, e.g., on the specific allele, genome, or target site to be edited; on the cell or tissue being targeted; and on the agent being used.

The term “immediately adjacent” as used in the context of two nucleic acid sequences refers to two sequences that directly abut each other as part of the same nucleic acid molecule and are not separated by one or more nucleotides. Accordingly, sequences are immediately adjacent, when the nucleotide at the 3′-end of one of the sequences is directly connected to nucleotide at the 5′-end of the other sequence via a phosphodiester bond.

The term “linker,” as used herein, refers to a chemical group or a molecule linking two molecules or moieties, e.g., two domains of a fusion protein, such as, for example, a nuclease-inactive Cas9 domain and a effector domain (e.g., a deaminase domain). In some embodiments, a linker joins a gRNA binding domain of an RNA-programmable nuclease, including a Cas9 nuclease domain, and the catalytic domain of a nucleic-acid editing protein. In some embodiments, a linker joins a dCas9 and a nucleic-acid editing protein. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, an RNA is an RNA associated with the Cas9 system. For example, the RNA may be a CRISPR RNA (crRNA), a trans-encoded small RNA (tracrRNA), a single guide RNA (sgRNA), or a guide RNA (gRNA).

The term “proliferative disease,” as used herein, refers to any disease in which cell or tissue homeostasis is disturbed in that a cell or cell population exhibits an abnormally elevated proliferation rate. Proliferative diseases include hyperproliferative diseases, such as pre-neoplastic hyperplastic conditions and neoplastic diseases. Neoplastic diseases are characterized by an abnormal proliferation of cells and include both benign and malignant neoplasias. Malignant neoplasia is also referred to as cancer.

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “RNA-programmable nuclease,” and “RNA-guided nuclease” are used interchangeably herein and refer to a nuclease that forms a complex with (e.g., binds or associates with) one or more RNA that is not a target for cleavage. In some embodiments, an RNA-programmable nuclease, when in a complex with an RNA, may be referred to as a nuclease: RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821 (2012), the entire contents of which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases and Uses Thereof,” and U.S. Provisional Patent Application, U.S. Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease: RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an MI strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roc B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature biotechnology 31, 227-229 (2013); Jinek, M. et al. RNA-programmed genome editing in human cells. eLife 2, c00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex, of any age, and at any stage of development.

The term “target site” refers to a sequence within a nucleic acid molecule that is deaminated by a deaminase or a fusion protein comprising a deaminase (e.g., a dCas9-deaminase fusion protein provided herein).

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “nucleic acid editing enzyme” as used herein refers to proteins that are able to modify a nucleic acids or one or more nucleotide bases of a nucleic acid. For example, in some embodiments, a nucleic acid editing enzyme is a deaminase, which can catalyze C to T or G to A changes. Other suitable nucleic acid editing enzyme that may be used in accordance with this disclosure include, without limitation, a nuclease, nickase, recombinase, deaminase, methyltransferase, methylase, acetylase, or acetyltransferase.

### DETAILED DESCRIPTION OF CERTAIN EMBODIMENTS OF THE INVENTION

Some aspects of this disclosure provide recombinant Cas9 proteins that efficiently target DNA sequences that do not comprise the canonical PAM sequence (5′-NGG-3′, where N is any nucleotide, for example A, T, G, or C) at their 3′-ends. In some embodiments, the Cas9 proteins provided herein comprise one or more mutations identified in directed evolution experiments using a target sequence library comprising randomized PAM sequences. The recombinant non-PAM restricted Cas9 proteins provided herein are useful for targeting DNA sequences that do not comprise the canonical PAM sequence at their 3′-end and thus greatly extend the usefulness of Cas9 technology for gene editing.

Some aspects of this disclosure provide fusion proteins that comprise a Cas9 protein and an effector domain, for example, a DNA-editing domain, such as, e.g., a deaminase domain. The deamination of a nucleobase by a deaminase can lead to a point mutation at the specific residue, which is referred to herein as nucleic acid editing. Fusion proteins comprising a Cas9 protein or variant thereof and a DNA-editing domain can thus be used for the targeted editing of nucleic acid sequences. Such fusion proteins are useful for targeted editing of DNA in vitro, e.g., for the generation of mutant cells or animals; for the introduction of targeted mutations, e.g., for the correction of genetic defects in cells ex vivo, e.g., in cells obtained from a subject that are subsequently re-introduced into the same or another subject; and for the introduction of targeted mutations, e.g., the correction of genetic defects or the introduction of deactivating mutations in disease-associated genes in a subject in vivo. Typically, the Cas9 protein of the fusion proteins described herein does not have any nuclease activity but instead is a Cas9 fragment or a dCas9 protein. Methods for the use of Cas9 fusion proteins as described herein are also provided.

Non-limiting, exemplary nuclease-inactive Cas9 proteins are provided herein. One exemplary suitable nuclease-inactive Cas9 protein is the D10A/H840A Cas9 protein mutant:

MDKKYSIGLAIGTNSVGWAVITDEYKVPSKKFKVLGNTDRHSIKKNLIGALLFDSGETAEATRLKRTARRRYTRRKN
RICYLQEIFSNEMAKVDDSFFHRLEESFLVEEDKKHERHPIFGNIVDEVAYHEKYPTIYHLRKKLVDSTDKADLRLI
YLALAHMIKFRGHFLIEGDLNPDNSDVDKLFIQLVQTYNQLFEENPINASGVDAKAILSARLSKSRRLENLIAQLPG
EKKNGLFGNLIALSLGLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQEEFYKFIKPILEKMDG
TEELLVKLNREDLLRKQRTEDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRENASLGTYHDLLKIIKDKDELDNEENEDIL
EDIVLTLTLFEDREMIEERLKTYAHLFDDKVMKQLKRRRYTGWGRLSRKLINGIRDKQSGKTILDFLKSDGFANRNE
MQLIHDDSLTFKEDIQKAQVSGQGDSLHEHIANLAGSPAIKKGILQTVKVVDELVKVMGRHKPENIVIEMARENQTT
QKGQKNSRERMKRIEEGIKELGSQILKEHPVENTQLQNEKLYLYYLQNGRDMYVDQELDINRLSDYDVDAIVPQSFL
KDDSIDNKVLTRSDKNRGKSDNVPSEEVVKKMKNYWRQLLNAKLITQRKEDNLTKAERGGLSELDKAGFIKRQLVET
RQITKHVAQILDSRMNTKYDENDKLIREVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEITLANGEIRKRPLIEINGETGEIVWDKGR
DFATVRKVLSMPQVNIVKKTEVQTGGFSKESILPKRNSDKLIARKKDWDPKKYGGFDSPTVAYSVLVVAKVEKGKSK
KLKSVKELLGITIMERSSFEKNPIDFLEAKGYKEVKKDLIIKLPKYSLFELENGRKRMLASAGELQKGNELALPSKY
VNFLYLASHYEKLKGSPEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD
(SEQ ID NO: 262; see, e.g., Qi et al., Repurposing CRISPR as an RNA-guided
platform for sequence-specific control of gene expression.
Cell. 2013; 152(5): 1173-83, the entire contents of which are incorporated
herein by reference).

Additional suitable nuclease-inactive Cas9 proteins will be apparent to those of skill in the art based on this disclosure. Such additional exemplary suitable nuclease-inactive Cas9 proteins include, but are not limited to, D10A, D839A, H840A, N863A, D10A/D839A, D10A/H840A, D10A/N863A, D839A/H840A, D839A/N863A, D10A/D839A/H840A, and D10A/D839A/H840A/N863A mutant proteins (See, e.g., Prashant et al., CAS9 transcriptional activators for target specificity screening and paired nickases for cooperative genome engineering. Nature Biotechnology. 2013; 31 (9): 833-838, the entire contents of which are incorporated herein by reference).

### Recombinant Cas9 Proteins

Some aspects of this disclosure provide recombinant Cas9 proteins comprising an amino acid sequence that is at least 80%, at least 85%, at least 90%, at least 92%, at least 95%, at least 96%, at least 97%, at least 98%, at least 99%, or at least 99.5% identical to the amino acid sequence of Cas9 as provided by any of the sequences provided in SEQ ID NOs: 10-262, wherein the Cas9 protein comprises a RuvC and an HNH domain, wherein the amino acid sequence of the Cas9 protein comprises at least one, at least two, at least three, at least four, at least five, at least six, or at least seven mutations at an amino acid residue selected from the group consisting of amino acid residues 122, 137, 182, 262, 294, 409, 480, 543, 660, 694, 1219, and 1329 of the amino acid sequence provided in SEQ ID NO: 9, or in a corresponding amino acid residue in any of the amino acid sequences provided in SEQ ID NOs: 10-262, and wherein the amino acid sequence of the recombinant Cas9 protein is not identical to the amino acid sequence of a naturally occurring Cas9 protein.

In some embodiments, the amino acid sequence of the Cas9 protein comprises at least one, at least two, at least three, at least four, at least five, at least six, or at least seven mutations selected from the group consisting of X262T, X294R, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid at the corresponding position.

In some embodiments, the amino acid sequence of the Cas9 protein comprises at least one, at least two, at least three, at least four, at least five, at least six, or at least seven mutations selected from the group consisting of A262T, K294R, S409I, E480K, E543D, M694I, or E1219V of the amino acid sequence provided in SEQ ID NO: 9, or a corresponding mutation in any of the amino acid sequences provided in SEQ ID NOs: 10-262. In some embodiments, the amino acid sequence of the Cas9 protein comprises an X1219V mutation of the amino acid sequence provided in SEQ ID NO: 9, or a corresponding mutation in any of the amino acid sequences provided in SEQ ID NOs: 10-262. In some embodiments, the amino acid sequence of the Cas9 protein comprises an E1219V mutation of the amino acid sequence provided in SEQ ID NO: 9, or a corresponding mutation in any of the amino acid sequences provided in SEQ ID NOs: 10-262.

In some embodiments, the amino acid sequence of the HNH domain is at least 80%, at least 85%, at least 90%, at least 92%, at least 95%, at least 96%, at least 97%, at least 98%, at least 99%, or at least 99.5% identical to the amino acid sequence of the HNH domain of SEQ ID NO: 9. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 10-262. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of SEQ ID NO: 9. In some embodiments, the Cas9 protein comprises a D10A and/or a H840A mutation of the amino acid sequence provided in SEQ ID NO: 9, or a corresponding mutation in any of the amino acid sequences provided in SEQ ID NOs: 10-262.

Recombinant Cas9 Proteins with Activity on Non-Canonical PAMs

Some aspects of this disclosure provide recombinant Cas9 proteins comprising an amino acid sequence that is at least 90% identical to the amino acid sequence of Streptococcus pyogenes Cas9 as provided by SEQ ID NO: 9, comprising the RuvC and HNH domains of SEQ ID NO: 9, wherein the amino acid sequence of the Cas9 protein comprises at least one, at least two, at least three, at least four, at least five, at least six, or at least seven mutations in an amino acid residue selected from the group consisting of amino acid residues 122, 137, 182, 262, 294, 409, 480, 543, 660, 694, 1219, and 1329 of the amino acid sequence provided in SEQ ID NO: 9, wherein the amino acid sequence of the recombinant Cas9 protein is not identical to the amino acid sequence of a naturally occurring Cas9 protein, and wherein the recombinant Cas9 protein exhibits activity (e.g., increased activity) on a target sequence that does not comprise the canonical PAM (5′-NGG-3′) at its 3′-end as compared to Streptococcus pyogenes Cas9 as provided by SEQ ID NO: 9.

In some embodiments, the Cas9 protein exhibits activity on a target sequence having a 3′-end that is not directly adjacent to the canonical PAM sequence (5′-NGG-3′) that is at least 2-fold, at least 3-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes Cas9 as provided by SEQ ID NO: 9 on the same target sequence. In some embodiments, the 3′-end of the target sequence is directly adjacent to an AGC, GAG, TTT, GTG, or CAA sequence. In some embodiments, the Cas9 protein activity is measured by a nuclease assay, a deamination assay, or a transcriptional activation assay. In some embodiments, the transcriptional activation assay is a reporter activation assay, such as a GFP activation assay. Exemplary methods for measuring binding activity (e.g., of Cas9) using transcriptional activation assays are known in the art and would be apparent to the skilled artisan. For example, methods for measuring Cas9 activity using the tripartite activator VPR have been described in Chavez A., et al., “Highly efficient Cas9-mediated transcriptional programming.” Nature Methods 12, 326-328 (2015); the entire contents of which are incorporated by reference herein.

In some embodiments, the amino acid sequence of the Cas9 protein comprises at least one, at least two, at least three, at least four, at least five, at least six, or at least seven mutations selected from the group consisting of X262T, X294R, X409I, X480K, X543D, X694I, and X1219V of the amino acid sequence provided in SEQ ID NO: 9, or a corresponding mutation in any of the amino acid sequences provided in SEQ ID NOs: 10-262, wherein X represents any amino acid at the corresponding position.

In some embodiments, the amino acid sequence of the Cas9 protein comprises at least one, at least two, at least three, at least four, at least five, at least six, or at least seven mutations selected from the group consisting of A262T, K294R, S409I, E480K, E543D, M694I, or E1219V of the amino acid sequence provided in SEQ ID NO: 9, or a corresponding mutation in any of the amino acid sequences provided in SEQ ID NOs: 10-262. In some embodiments, the amino acid sequence of the Cas9 protein comprises an X1219V mutation of the amino acid sequence provided in SEQ ID NO: 9, or a corresponding mutation in any of the amino acid sequences provided in SEQ ID NOs: 10-262. In some embodiments, the amino acid sequence of the Cas9 comprises an X1219V mutation of the amino acid sequence provided in SEQ ID NO: 9, or a corresponding mutation in any of the amino acid sequences provided in SEQ ID NOs: 10-262.

In some embodiments, the amino acid sequence of the HNH domain is at least 80%, at least 85%, at least 90%, at least 92%, at least 95%, at least 96%, at least 97%, at least 98%, at least 99%, or at least 99.5% identical to the amino acid sequence of the HNH domain of any of SEQ ID NOs: 2, 4, or 9. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 10-262. In some embodiments, the Cas9 protein comprises a D10A and/or a H840A mutation of the amino acid sequence provided in SEQ ID NO: 9, or a corresponding mutation in any of the amino acid sequences provided in SEQ ID NOs: 10-262.

Some aspects of this disclosure provide recombinant Cas9 proteins comprising an amino acid sequence that is at least 90% identical to the amino acid sequence of Streptococcus pyogenes Cas9 as provided by SEQ ID NO: 9, comprising the RuvC and HNH domains of SEQ ID NO: 9, wherein the amino acid sequence of the Cas9 protein comprises at least one, at least two, at least three, at least four, at least five, at least six, or at least seven mutations in an amino acid residue selected from the group consisting of amino acid residues 122, 137, 182, 262, 294, 409, 480, 543, 660, 694, 1219, and 1329 of the amino acid sequence provided in SEQ ID NO: 9, wherein the amino acid sequence of the recombinant Cas9 protein is not identical to the amino acid sequence of a naturally occurring Cas9 protein, and wherein the recombinant Cas9 protein exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 2, 4, or 9. In some embodiments, the Cas9 protein comprises a D10A and a H840A mutation of the amino acid sequence provided in SEQ ID NO: 9, or a corresponding mutation in any of the amino acid sequences provided in SEQ ID NOs: 10-262.

Some aspects of this disclosure provide recombinant Cas9 proteins comprising an amino acid sequence that is at least 90% identical to the amino acid sequence of Streptococcus pyogenes Cas9 as provided by SEQ ID NO: 9, comprising the RuvC and HNH domains of SEQ ID NO: 9, wherein the amino acid sequence of the Cas9 protein comprises at least one, at least two, at least three, at least four, at least five, at least six, or at least seven mutations in an amino acid residue selected from the group consisting of amino acid residues 122, 137, 182, 262, 294, 409, 480, 543, 660, 694, 1219, and 1329 of the amino acid sequence provided in SEQ ID NO: 9, wherein the amino acid sequence of the recombinant Cas9 protein is not identical to the amino acid sequence of a naturally occurring Cas9 protein, and wherein the recombinant Cas9 protein exhibits an increased activity on a target sequence that does not comprise the canonical PAM (5′-NGG-3′) at its 3′-end as compared to Streptococcus pyogenes Cas9 as provided by SEQ ID NO: 9. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 2, 4, or 9. In some embodiments, the Cas9 protein comprises a D10A mutation of the amino acid sequence provided in SEQ ID NO: 9, or a corresponding mutation in any of the amino acid sequences provided in SEQ ID NOs: 10-262. In some embodiments, the Cas9 further comprises a histidine residue at position 840 as provided in SEQ ID NO: 9, or a corresponding histidine residue in any of the amino acid sequences provided in SEQ ID NOs: 10-262. Without wishing to be bound by any particular theory, the presence of the catalytic residue H840 allows Cas9 to cleave the non-targeted strand, i.e., the strand bound by the sgRNA. In some embodiments, a Cas9 having an amino acid residue other than histidine at position 840 of the amino acid sequence provided in SEQ ID NO: 9, or a corresponding amino acid sequence provided in SEQ ID NOs: 10-262 may be changed or reverted such that amino acid residue 840 of the amino acid sequence provided in SEQ ID NO: 9, or a corresponding amino acid sequence provided in SEQ ID NOs: 10-262 is histidine.

Some aspects of this disclosure provide recombinant Cas9 proteins comprising an amino acid sequence that is at least 90% identical to the amino acid sequence of Streptococcus pyogenes Cas9 as provided by SEQ ID NO: 9, comprising the RuvC and HNH domains of SEQ ID NO: 9; wherein the amino acid sequence of the Cas9 protein comprises at least one, at least two, at least three, at least four, at least five, at least six, or at least seven mutations in an amino acid residue selected from the group consisting of amino acid residues 262, 267, 294, 405, 409, 480, 543, 694, 1219, 1224, and 1256 of the amino acid sequence provided in SEQ ID NO: 9; wherein the amino acid sequence of the recombinant Cas9 protein is not identical to the amino acid sequence of a naturally occurring Cas9 protein; and wherein the recombinant Cas9 protein exhibits activity (e.g., increased activity) on a target sequence that does not comprise the canonical PAM (5′-NGG-3′) at its 3′-end as compared to Streptococcus pyogenes Cas9 as provided by SEQ ID NO: 9.

In some embodiments, the Cas9 protein exhibits an activity on a target sequence having a 3′-end that is not directly adjacent to the canonical PAM sequence (5′-NGG-3′) that is at least 2-fold, at least 3-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the activity of Streptococcus pyogenes Cas9 as provided by SEQ ID NO: 9 on the same target sequence. In some embodiments, the 3′-end of the target sequence is directly adjacent to an AAA, AAC, AAG, AAT, CAA, CAC, CAG, CAT, GAA, GAC, GAG, GAT, TAA, TAC, TAG, TAT, ACA, ACC, ACG, ACT, CCA, CCC, CCG, CCT, GCA, GCC, GCG, GCT, TCA, TCC, TCG, TCT, AGA, AGC, AGT, CGA, CGC, CGT, GGA, GGC, GGT, TGA, TGC, TGT, ATA, ATC, ATG, ATT, CTA, CTC, CTG, CTT, GTA, GTC, GTG, GTT, TTA, TTC, TTG, or TTT PAM sequence.

In some embodiments, the amino acid sequence of the Cas9 protein comprises at least one, at least two, at least three, at least four, at least five, at least six, or at least seven mutations selected from the group consisting of X262T, X267G, X294R, X405I, X409I, X480K, X543D, X694I, X1219V, X1224K, X1256K, and X1362P of the amino acid sequence provided in SEQ ID NO: 9, or a corresponding mutation in any of the amino acid sequences provided in SEQ ID NOs: 10-262, wherein X represents any amino acid at the corresponding position.

In some embodiments, the amino acid sequence of the Cas9 protein comprises at least one, at least two, at least three, at least four, at least five, at least six, or at least seven mutations selected from the group consisting of A262T, S267G, K294R, F405I, S409I, E480K, E543D, M694I, E1219V, N1224K, Q1256K, and L1362P of the amino acid sequence provided in SEQ ID NO: 9, or a corresponding mutation in any of the amino acid sequences provided in SEQ ID NOs: 10-262. In some embodiments, the amino acid sequence of the Cas9 protein comprises an X1219V mutation of the amino acid sequence provided in SEQ ID NO: 9, or a corresponding mutation in any of the amino acid sequences provided in SEQ ID NOs: 10-262. In some embodiments, the amino acid sequence of the Cas9 protein comprises an E1219V mutation of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises an X480K mutation of the amino acid sequence provided in SEQ ID NO: 9, or a corresponding mutation in any of the amino acid sequences provided in SEQ ID NOs: 10-262. In some embodiments, the amino acid sequence of the Cas9 protein comprises an E480K mutation of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises an X543D mutation of the amino acid sequence provided in SEQ ID NO: 9, or a corresponding mutation in any of the amino acid sequences provided in SEQ ID NOs: 10-262. In some embodiments, the amino acid sequence of the Cas9 protein comprises an E543D mutation of the amino acid sequence provided in SEQ ID NO: 9, or a corresponding mutation in any of the amino acid sequences provided in SEQ ID NOs: 10-262.

In some embodiments, the amino acid sequence of the Cas9 protein comprises the combination of mutations selected from the group consisting of (X480K, X543D, and X1219V); (X262T, X409I, X480K, X543D, X694I, and X1219V); (X294R, X480K, X543D, X1219V, X1256K, and X1362P); (X294R, X480K, X543D, X1219V, and X1256K); (X267G, X294R, X480K, X543D, X1219V, X1224K, and X1256K); and (X262T, X405I, X409I, X480K, X543D, X694I, and X1219V) of the amino acid sequence provided in SEQ ID NO: 9, or a corresponding mutation in any of the amino acid sequences provided in SEQ ID NOs: 10-262. In some embodiments, the amino acid sequence of the Cas9 protein comprises the combination of mutations selected from the group consisting of (E480K, E543D, and E1219V); (A262T, S409I, E480K, E543D, M694I, and E1219V); (K294R, E480K, E543D, E1219V, Q1256K, and L1362P); (K294R, E480K, E543D, E1219V, and Q1256K); (S267G, K294R, E480K, E543DE1219V, N1224K, and Q1256K); and (A262T, F405I, S409I, E480K, E543D, M694I, and E1219V) of the amino acid sequence provided in SEQ ID NO: 9, or a corresponding mutation in any of the amino acid sequences provided in SEQ ID NOs: 10-262.

In some embodiments, the amino acid sequence of the HNH domain is at least 80%, at least 85%, at least 90%, at least 92%, at least 95%, at least 96%, at least 97%, at least 98%, at least 99%, or at least 99.5% identical to the amino acid sequence of the HNH domain of any of SEQ ID NOs: 2, 4, or 9. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 2, 4, or 9. In some embodiments, the Cas9 protein comprises a D10A and/or a H840A mutation of the amino acid sequence provided in SEQ ID NO: 9, or a corresponding mutation in any of the amino acid sequences provided in SEQ ID NOs: 10-262. In some embodiments, the Cas9 protein comprises a D10A and an H840A mutation of the amino acid sequence provided in SEQ ID NO: 9, or a corresponding mutation in any of the amino acid sequences provided in SEQ ID NOs: 10-262. In some embodiments, the Cas9 protein comprises a D10A mutation of the amino acid sequence provided in SEQ ID NO: 9, or a corresponding mutation in any of the amino acid sequences provided in SEQ ID NOs: 10-262. In some embodiments, the Cas9 protein comprises an H840A mutation of the amino acid sequence provided in SEQ ID NO: 9, or a corresponding mutation in any of the amino acid sequences provided in SEQ ID NOs: 10-262.

Some aspects of this disclosure provide recombinant Cas9 proteins comprising an amino acid sequence that is at least 90% identical to the amino acid sequence of Streptococcus pyogenes Cas9 as provided by SEQ ID NO: 9, comprising the RuvC and HNH domains of SEQ ID NO: 9, wherein the amino acid sequence of the Cas9 protein comprises at least one, at least two, at least three, at least four, at least five, at least six, or at least seven mutations in an amino acid residue selected from the group consisting of amino acid residues 262, 267, 294, 405, 409, 480, 543, 694, 1219, 1224, 1256, and 1362 of the amino acid sequence provided in SEQ ID NO: 9, wherein the amino acid sequence of the recombinant Cas9 protein is not identical to the amino acid sequence of a naturally occurring Cas9 protein, and wherein the recombinant Cas9 protein exhibits increased activity on a target sequence that does not include the canonical PAM (5′-NGG-3′) at its 3′-end as compared to Streptococcus pyogenes Cas9 as provided by SEQ ID NO: 9. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 2, 4, or 9. In some embodiments, the Cas9 protein comprises a D10A and a H840A mutation of the amino acid sequence provided in SEQ ID NO: 9, or a corresponding mutation in any of the amino acid sequences provided in SEQ ID NOs: 10-262.

Some aspects of this disclosure provide recombinant Cas9 proteins comprising an amino acid sequence that is at least 90% identical to the amino acid sequence of Streptococcus pyogenes Cas9 as provided by SEQ ID NO: 9, comprising the RuvC and HNH domains of SEQ ID NO: 9, wherein the amino acid sequence of the Cas9 protein comprises at least one, at least two, at least three, at least four, at least five, at least six, or at least seven mutations at an amino acid residue selected from the group consisting of amino acid residues 262, 267, 294, 405, 409, 480, 543, 694, 1219, 1224, 1256, and 1362 of the amino acid sequence provided in SEQ ID NO: 9, wherein the amino acid sequence of the recombinant Cas9 protein is not identical to the amino acid sequence of a naturally occurring Cas9 protein, and wherein the recombinant Cas9 protein exhibits increased activity on a target sequence that does not comprise the canonical PAM (5′-NGG-3′) at its 3′-end as compared to Streptococcus pyogenes Cas9 as provided by SEQ ID NO: 9. In some embodiments, the amino acid sequence of the RuvC domain is at least 80%, at least 85%, at least 90%, at least 92%, at least 95%, at least 96%, at least 97%, at least 98%, at least 99%, or at least 99.5% identical to the amino acid sequence of the RuvC domain of any of SEQ ID NOs: 2, 4, or 9. In some embodiments, the Cas9 protein comprises a D10A mutation of the amino acid sequence provided in SEQ ID NO: 9, or a corresponding mutation in any of the amino acid sequences provided in SEQ ID NOs: 10-262. In some embodiments, the Cas9 further comprises a histidine residue at position 840 as provided in SEQ ID NO: 9, or a corresponding histidine residue in any of the amino acid sequences provided in SEQ ID NOs: 10-262. Without wishing to be bound by any particular theory, the presence of the catalytic residue H840 allows Cas9 to cleave the non-targeted strand, i.e., the strand bound by the sgRNA. In some embodiments, a Cas9 having an amino acid residue other than histidine at position 840 of the amino acid sequence provided in SEQ ID NO: 9, or a corresponding amino acid sequence provided in SEQ ID NOs: 10-262 may be changed or reverted such that amino acid residue 840 of the amino acid sequence provided in SEQ ID NO: 9, or a corresponding amino acid sequence provided in SEQ ID NOs: 10-262 is histidine.

### Cas9 Fusion Proteins

Some aspects of this disclosure provide fusion proteins comprising a Cas9 protein as provided herein that is fused to a second protein, or a “fusion partner”, such as an effector domain, thus forming a fusion protein. In some embodiments, the effector domain is fused to the N-terminus of the Cas9 protein. In some embodiments, the effector domain is fused to the C-terminus of the Cas9 protein. In some embodiments, the Cas9 protein and the effector domain are fused to each other via a linker. Suitable strategies for generating fusion proteins according to aspects of this disclosure using linkers or without the use of linkers will also be apparent to those of skill in the art in view of the instant disclosure and the knowledge in the art. For example, Gilbert et al., CRISPR-mediated modular RNA-guided regulation of transcription in eukaryotes. Cell. 2013; 154 (2): 442-51, showed that C-terminal fusions of Cas9 with VP64 using 2 NLS's as a linker (SPKKKRKVEAS, SEQ ID NO: 284), can be employed for transcriptional activation. Mali et al., CAS9 transcriptional activators for target specificity screening and paired nickases for cooperative genome engineering. Nat Biotechnol. 2013; 31 (9): 833-8, reported that C-terminal fusions with VP64 without linker can be employed for transcriptional activation. And Maeder et al., CRISPR RNA-guided activation of endogenous human genes. Nat Methods. 2013; 10:977-979, reported that C-terminal fusions with VP64 using a Gly Ser (SEQ ID NO: 5) linker can be used as transcriptional activators. Recently, dCas9-FokI nuclease fusions have successfully been generated and exhibit improved enzymatic specificity as compared to the parental Cas9 enzyme (In Guilinger J P, Thompson D B, Liu D R. Fusion of catalytically inactive Cas9 to FokI nuclease improves the specificity of genome modification. Nat. Biotechnol. 2014; 32 (6): 577-82, and in Tsai S Q, Wyvekens N, Khayter C, Foden J A, Thapar V, Reyon D, Goodwin M J, Aryec M J, Joung J K. Dimeric CRISPR RNA-guided FokI nucleases for highly specific genome editing. Nat Biotechnol. 2014; 32 (6): 569-76. PMID: 24770325 a SGSETPGTSESATPES (SEQ ID NO: 7) or a GGGGSₙ (SEQ ID NO: 5) linker was used in FokI-dCas9 fusion proteins, respectively). In some embodiments, the linker comprises a (GGGGS), (SEQ ID NO: 5), a (G)ₙ (SEQ ID NO: 5087), an (EAAAK)ₙ (SEQ ID NO: 6), a (GGS)ₙ (SEQ ID NO: 5088), an SGSETPGTSESATPES (SEQ ID NO: 7), or an (XP)ₙ (SEQ ID NO: 5089) motif, or a combination of any of these, wherein n is independently an integer between 1 and 30. In some embodiments, the effector domain comprises an enzymatic domain. Suitable effector domains include, without limitation a nuclease, nickase, recombinase, deaminase, methyltransferase, methylase, acetylase, acetyltransferase, transcriptional activator, and transcriptional repressor.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the effector domain comprises a effector enzyme. Suitable effector enzymes that may be used in accordance with this disclosure include nucleases, nickases, recombinases, and deaminases. However additional effector enzymes would be apparent to the skilled artisan and are within the scope of this disclosure. In other embodiments, the effector domain comprises a domain that modulates transcriptional activity. Such transcriptional modulating domains may be, without limitation, a transcriptional activator or transcriptional repressor domain.

In some embodiments, the effector domain is a effector domain. In some embodiments, the effector domain is a deaminase domain. In some embodiments, the deaminase is a cytosine deaminase or a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID).

In some embodiments, the effector domain is at least 80%, at least 85%, at least 90%, at least 92%, at least 95%, at least 96%, at least 97%, at least 98%, at least 99%, or at least 99.5% identical to the deaminase domain of any one of SEQ ID NOs: 263-281.

In some embodiments, the effector domain is a nuclease domain. In some embodiments, the nuclease domain is a FokI DNA cleavage domain. In some embodiments, this disclosure provides dimers of the fusion proteins provided herein, e.g., dimers of fusion proteins may include a dimerizing nuclease domain.

In some embodiments, the Cas9 protein comprises an amino acid sequence that is at least 80%, at least 85%, at least 90%, at least 92%, at least 95%, at least 96%, at least 97%, at least 98%, at least 99%, or at least 99.5% identical to the amino acid sequence of Cas9 as provided by any of the sequences provided in SEQ ID NOs: 10-262, wherein the Cas9 protein comprises a RuvC and an HNH domain, wherein the amino acid sequence of the Cas9 protein comprises at least one, at least two, at least three, at least four, at least five, at least six, or at least seven mutations in an amino acid residue selected from the group consisting of amino acid residues 122, 137, 182, 262, 294, 409, 480, 543, 660, 694, 1219, and 1329 of the amino acid sequence provided in SEQ ID NO: 9, or in a corresponding amino acid residue in any of the amino acid sequences provided in SEQ ID NOs: 10-262, and wherein the amino acid sequence of the recombinant Cas9 protein is not identical to the amino acid sequence of a naturally occurring Cas9 protein. In some embodiments, the Cas9 protein comprises an amino acid sequence that is at least 90% identical to the amino acid sequence of Streptococcus pyogenes Cas9 as provided by SEQ ID NO: 9, comprising the RuvC and HNH domains of SEQ ID NO: 9, wherein the amino acid sequence of the Cas9 protein comprises at least one, at least two, at least three, at least four, at least five, at least six, or at least seven mutations in an amino acid residue selected from the group consisting of amino acid residues 122, 137, 182, 262, 294, 409, 480, 543, 660, 694, 1219, and 1329 of the amino acid sequence provided in SEQ ID NO: 9, wherein the amino acid sequence of the recombinant Cas9 protein is not identical to the amino acid sequence of a naturally occurring Cas9 protein, and wherein the recombinant Cas9 protein exhibits activity on a target sequence that does not comprise the canonical PAM (5′-NGG-3′) at its 3′-end as compared to Streptococcus pyogenes Cas9 as provided by SEQ ID NO: 9.

In some embodiments, the Cas9 protein comprises an amino acid sequence that is at least 80%, at least 85%, at least 90%, at least 92%, at least 95%, at least 96%, at least 97%, at least 98%, at least 99%, or at least 99.5% identical to the amino acid sequence of Cas9 as provided by any of the sequences provided in SEQ ID NOs: 10-262, wherein the Cas9 protein comprises a RuvC and an HNH domain, wherein the amino acid sequence of the Cas9 protein comprises at least one, at least two, at least three, at least four, at least five, at least six, or at least seven mutations in an amino acid residue selected from the group consisting of amino acid residues 262, 267, 294, 405, 409, 480, 543, 694, 1219, 1224, 1256, and 1362 of the amino acid sequence provided in SEQ ID NO: 9, or in a corresponding amino acid residue in any of the amino acid sequences provided in SEQ ID NOs: 10-262, and wherein the amino acid sequence of the recombinant Cas9 protein is not identical to the amino acid sequence of a naturally occurring Cas9 protein. In some embodiments, the Cas9 protein comprises an amino acid sequence that is at least 90% identical to the amino acid sequence of Streptococcus pyogenes Cas9 as provided by SEQ ID NO: 9, comprising the RuvC and HNH domains of SEQ ID NO: 9, wherein the amino acid sequence of the Cas9 protein comprises at least one, at least two, at least three, at least four, at least five, at least six, or at least seven mutations in an amino acid residue selected from the group consisting of amino acid residues 262, 267, 294, 405, 409, 480, 543, 694, 1219, 1224, and 1256 of the amino acid sequence provided in SEQ ID NO: 9; wherein the amino acid sequence of the recombinant Cas9 protein is not identical to the amino acid sequence of a naturally occurring Cas9 protein; and wherein the recombinant Cas9 protein exhibits increased activity on a target sequence that does not include the canonical PAM (5′-NGG-3′) at its 3′-end, as compared to Streptococcus pyogenes Cas9 as provided by SEQ ID NO: 9.

Some aspects of this disclosure provide fusion proteins comprising (i) a nuclease-inactive Cas9 protein; and (ii) a effector domain. In some embodiments, the effector domain is a DNA-editing domain. In some embodiments, the effector domain possesses deaminase activity. In some embodiments, the effector domain comprises or is a deaminase domain. In some embodiments, the deaminase is a cytidine deaminase. In some embodiments, the deaminase is an apolipoprotein B mRNA-editing complex (APOBEC) family deaminase. In some embodiments, the deaminase is an APOBEC1 family deaminase. In some embodiments, the deaminase is an activation-induced cytidine deaminase (AID). Some nucleic-acid editing domains as well as Cas9 fusion proteins including such domains are described in detail herein. Additional suitable effector domains will be apparent to the skilled artisan based on this disclosure. In some embodiments, the nucleic-acid editing domain is a FokI nuclease domain.

The instant disclosure provides Cas9:effector domain fusion proteins of various configurations. In some embodiments, the effector domain is fused to the N-terminus of the Cas9 protein. In some embodiments, the effector domain is fused to the C-terminus of the Cas9 protein. In some embodiments, the Cas9 protein and the effector domain are fused via a linker. In some embodiments, the linker comprises (GGGGS)ₙ (SEQ ID NO: 5), (G)ₙ (SEQ ID NO: 5087), (EAAAK)ₙ (SEQ ID NO: 6), (GGS)ₙ (SEQ ID NO: 5088), or SGSETPGTSESATPES (SEQ ID NO: 7) motif (see, e.g., Guilinger J P, Thompson D B, Liu D R. Fusion of catalytically inactive Cas9 to FokI nuclease improves the specificity of genome modification. Nat. Biotechnol. 2014; 32 (6): 577-82; the entire contents are incorporated herein by reference), or an (XP), (SEQ ID NO: 5089) motif, or a combination of any of these, wherein n is independently an integer between 1 and 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65 (10): 1357-69, the entire contents of which are incorporated herein by reference. Additional suitable linker sequences will be apparent to those of skill in the art based on the instant disclosure and knowledge in the art.

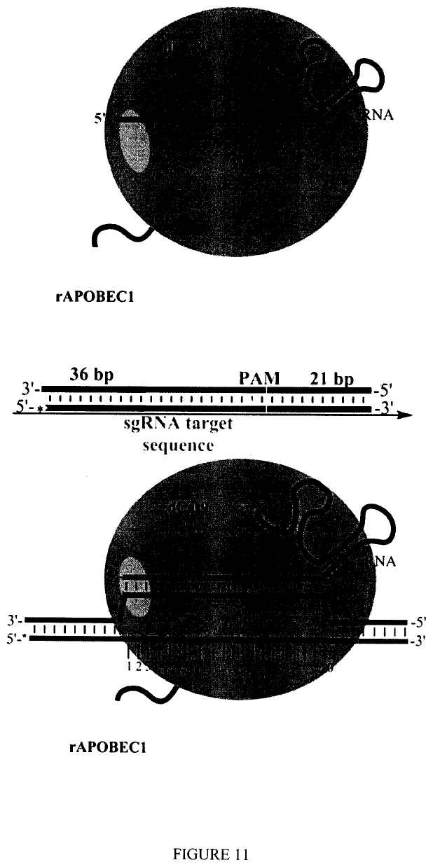

In some embodiments, the general architecture of exemplary Cas9 fusion proteins provided herein comprises the structure:
[NH₂]-[effector domain]-[Cas9]-[COOH] or
   [NH₂]-[Cas9]-[effector domain]-[COOH],
      wherein NH₂ is the N-terminus of the fusion protein, and COOH is the C-terminus of the fusion protein. FIG. 11 provides a schematic representation of a Cas9 protein fused to an effector domain (e.g., rAPOBEC1) in complex with sgRNA and bound to the target nucleic acid sequence.

In some embodiments, any of the fusion proteins provided herein may comprise one or more nuclear localization sequence (NLS). As used herein, a nuclear localization sequence refers to an amino acid sequence that promotes importation of a protein, for example any of the fusion proteins provided herein having an NLS, into the cell nucleus (e.g., via nuclear transport). Typically, an NLS comprises one or more short amino acid sequences of positively charged lysines or arginines exposed on the protein surface. Nuclear localization sequences are known in the art and would be apparent to the skilled artisan. For example nuclear localization sequences have been described in Kalderon D., et al., “A short amino acid sequence able to specify nuclear location”. Cell (1984) 39 (3 Pt 2): 499-509; Dingwall C., et al., “The nucleoplasmin nuclear location sequence is larger and more complex than that of SV-40 large T antigen”. J Cell Biol. (1988) 107 (3): 841-9; Makkerh J. P., et al., “Comparative mutagenesis of nuclear localization signals reveals the importance of neutral and acidic amino acids”. Curr Biol. (1996) 6 (8): 1025-7; and Ray M., et al., “Quantitative tracking of protein trafficking to the nucleus using cytosolic protein delivery by nanoparticle-stabilized nanocapsules”. Bioconjug. Chem. (2015) 26 (6): 1004-7; the entire contents of each are incorporated by reference herein. Additional nuclear localization sequences are described, for example, in Plank et al., PCT/EP2000/011690, the entire contents are incorporated by reference herein. In some embodiments, a NLS comprises the amino acid sequence PKKKRKV (SEQ ID NO: 299) or MDSLLMNRRKFLYQFKNVRWAKGRRETYLC (SEQ ID NO: 300).

Exemplary features that may be present are localization sequences, such as nuclear localization sequences, cytoplasmic localization sequences, export sequences, such as nuclear export sequences, or other localization sequences, as well as sequence tags that are useful for solubilization, purification, or detection of the fusion proteins. Suitable localization signal sequences and sequences of protein tags are provided herein, and include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art and are within the scope of this disclosure.

Any of the nuclear localization sequences provided herein may be fused to the fusion protein in any suitable localization. For example, to promote translocation of the fusion protein into a cell nucleus without compromising function of the fusion protein. In some embodiments, the NLS is fused N-terminal to the Cas9 protein of the fusion protein. In some embodiments, the NLS is fused C-terminal to the Cas9 protein of the fusion protein. In some embodiments, the NLS is fused N-terminal to the effector domain of the fusion protein. In some embodiments, the NLS is fused C-terminal to the effector domain of the fusion protein.

In some embodiments, the effector domain is a deaminase. For example, in some embodiments, the general architecture of exemplary Cas9 fusion proteins with a deaminase domain comprises the structure:
[NH₂]-[NLS]-[Cas9]-[deaminase]-[COOH],
[NH₂]-[NLS]-[deaminase]-[Cas9]-[COOH],
[NH₂]-[Cas9]-[NLS]-[deaminase]-[COOH],
[NH₂]-[deaminase]-[NLS]-[Cas9]-[COOH],
[NH₂]-[deaminase]-[Cas9]-[NLS]-[COOH], or
[NH₂]-[Cas9]-[deaminase]-[NLS]-[COOH],
   wherein NLS is a nuclear localization signal, NH₂ is the N-terminus of the fusion protein, and COOH is the C-terminus of the fusion protein. In some embodiments, a linker is inserted between the Cas9 protein and the deaminase domain. In some embodiments, any of the “]-[” may be one or more linkers. In some embodiments, the NLS is located C-terminal of the deaminase and/or the Cas9 domain. In some embodiments, the NLS is located between the deaminase and the Cas9 domain. Additional features, such as sequence tags, may also be present.

One exemplary suitable type of effector domain includes cytosine deaminases, for example, of the APOBEC family. The apolipoprotein B mRNA-editing complex (APOBEC) family of cytosine deaminase enzymes encompasses eleven proteins that serve to initiate mutagenesis in a controlled and beneficial manner.²⁹ One family member, activation-induced cytidine deaminase (AID), is responsible for the maturation of antibodies by converting cytosines in ssDNA to uracils in a transcription-dependent, strand-biased fashion.³⁰ The apolipoprotein B editing complex 3 (APOBEC3) enzyme provides protection to human cells against a certain HIV-1 strain via the deamination of cytosines in reverse-transcribed viral SSDNA.³¹ These proteins all require a Zn²⁺-coordinating motif (His-X-Glu-X₂₃₋₂₆-Pro-Cys-X₂₋₄-Cys; SEQ ID NO: 283) and bound water molecule for catalytic activity. The Glu residue acts to activate the water molecule to a zinc hydroxide for nucleophilic attack in the deamination reaction. Each family member preferentially deaminates at its own particular “hotspot,” ranging from WRC (W is A or T, R is A or G) for hAID, to TTC for hAPOBEC3F.³² A recent crystal structure of the catalytic domain of APOBEC3G revealed a secondary structure comprised of a five-stranded β-sheet core flanked by six α-helices, which is believed to be conserved across the entire family.³³ The active center loops have been shown to be responsible for both ssDNA binding and in determining “hotspot” identity.³⁴ Overexpression of these enzymes has been linked to genomic instability and cancer, thus highlighting the importance of sequence-specific targeting.³⁵

Some aspects of this disclosure provide a systematic series of fusions between Cas9 and deaminase domains, e.g., cytosine deaminase enzymes such as APOBEC enzymes, or adenosine deaminase enzymes such as ADAT enzymes, that has been generated in order to direct the enzymatic activities of these deaminases to a specific site in genomic DNA. The advantages of using Cas9 as the recognition agent are two-fold: (1) the sequence specificity of Cas9 can be easily altered by simply changing the sgRNA sequence; and (2) Cas9 binds to its target sequence by denaturing the dsDNA, resulting in a stretch of DNA that is single-stranded and therefore a viable substrate for the deaminase. It will be understood that other catalytic domains, or catalytic domains from other deaminases, can also be used to generate fusion proteins with Cas9, and that the disclosure is not limited in this regard.

Some aspects of this disclosure are based on the recognition that cas9: deaminase fusion proteins can efficiently deaminate nucleotides at positions 3-11 according to the numbering scheme in FIG. 11. It should be appreciated that a person of skill in the art will be able to design suitable guide RNAs to target the fusion proteins to a target sequence that comprises a nucleotide to be deaminated. Both PAM-dependent Cas9 proteins or Cas9 proteins that can target PAM-less target sequences as provided herein, can be employed for deamination of a target nucleotide.

Some exemplary suitable nucleic-acid editing domains, e.g., deaminases and deaminase domains, that can be fused to Cas9 domains according to aspects of this disclosure are provided below. Typically, deaminase require a Zn²⁺-coordinating motif (His-X-Glu-X₂₃₋₂₆-Pro-Cys-X₂₋₄-Cys; SEQ ID NO: 283) and bound water molecule for catalytic activity. The Glu residue acts to activate the water molecule to a zinc hydroxide for nucleophilic attack in the deamination reaction. It will be understood that, in some embodiments, the active domain of the respective sequence can be used, e.g., the domain without a localizing signal (nuclear localizing signal, without nuclear export signal, cytoplasmic localizing signal).

Human AID:
(SEQ ID NO: 263)
MDSLLMNRRKFLYQFKNVRWAKGRRETYLCYVVKRRDSATSFSLDFGYLRNKNGCHV
ELLFLRYISDWDLDPGRCYRVTWFTSWSPCYDCARHVADFLRGNPNLSLRIFTARLYFC
EDRKAEPEGLRRLHRAGVQIAIMTFKDYFYCWNTFVENHERTFKAWEGLHENSVRLSR
QLRRILLPLYEVDDLRDAFRTLGL
(underline: nuclear localization signal; double underline:
nuclear export signal)
Mouse AID:
(SEQ ID NO: 264)
MDSLLMKQKKFLYHFKNVRWAKGRHETYLCYVVKRRDSATSCSLDFGHLRNKSGCHV
ELLFLRYISDWDLDPGRCYRVTWFTSWSPCYDCARHVAEFLRWNPNLSLRIFTARLYFC
EDRKAEPEGLRRLHRAGVQIGIMTFKDYFYCWNTFVENRERTFKAWEGLHENSVRLTR
QLRRILLPLYEVDDLRDAFRMLGE
(underline: nuclear localization signal; double underline:
nuclear export signal)
Dog AID:
(SEQ ID NO: 265)
MDSLLMKQRKFLYHFKNVRWAKGRHETYLCYVVKRRDSATSFSLDFGHLRNKSGCHV
ELLFLRYISDWDLDPGRCYRVTWFTSWSPCYDCARHVADFLRGYPNLSLRIFAARLYFC
EDRKAEPEGLRRLHRAGVQIAIMTFKDYFYCWNTFVENREKTFKAWEGLHENSVRLSR
QLRRILLPLYEVDDLRDAFRTLGL
(underline: nuclear localization signal; double underline:
nuclear export signal)
Bovine AID:
(SEQ ID NO: 266)
MDSLLKKQRQFLYQFKNVRWAKGRHETYLCYVVKRRDSPTSFSLDFGHLRNKAGCHV
ELLFLRYISDWDLDPGRCYRVTWFTSWSPCYDCARHVADFLRGYPNLSLRIFTARLYFC
DKERKAEPEGLRRLHRAGVQIAIMTFKDYFYCWNTFVENHERTFKAWEGLHENSVRLS
RQLRRILLPLYEVDDLRDAFRTLGL
(underline: nuclear localization signal; double underline:
nuclear export signal)
Mouse APOBEC-3:
(SEQ ID NO: 267)
MGPFCLGCSHRKCYSPIRNLISQETFKFHFKNLGYAKGRKDTFLCYEVTRKDCDSPVSLH
HGVFKNKDNIHAEICFLYWFHDKVLKVLSPREEFKITWYMSWSPCFECAEQIVRFLATHHN
LSLDIFSSRLYNVQDPETQQNLCRLVQEGAQVAAMDLYEFKKCWKKFVDNGGRRFRP
WKRLLTNFRYQDSKLQEILRPCYIPVPSSSSSTLSNICLTKGLPETRFCVEGRRMDPLSEE
EFYSQFYNQRVKHLCYYHRMKPYLCYQLEQFNGQAPLKGCLLSEKGKQHAEILFLDKIR
SMELSQVTITCYLTWSPCPNCAWQLAAFKRDRPDLILHIYTSRLYFHWKRPFQKGLCSLW
QSGILVDVMDLPQFTDCWTNFVNPKRPFWPWKGLEIISRRTQRRLRRIKESWGLQDLVN
DFGNLQLGPPMS
(italic: nucleic acid editing domain)
Rat APOBEC-3:
(SEQ ID NO: 268)
MGPFCLGCSHRKCYSPIRNLISQETFKFHFKNLRYAIDRKDTFLCYEVTRKDCDSPVSLH
HGVFKNKDNIHAEICFLYWFHDKVLKVLSPREEFKITWYMSWSPCFECAEQVLRFLATHHN
LSLDIFSSRLYNIRDPENQQNLCRLVQEGAQVAAMDLYEFKKCWKKFVDNGGRRFRPW
KKLLTNFRYQDSKLQEILRPCYIPVPSSSSSTLSNICLTKGLPETRFCVERRRVHLLSEEEF
YSQFYNQRVKHLCYYHGVKPYLCYQLEQFNGQAPLKGCLLSEKGKQHAEILFLDKIRSM
ELSQVIITCYLTWSPCPNCAWQLAAFKRDRPDLILHIYTSRLYFHWKRPFQKGLCSLWQSG
ILVDVMDLPQFTDCWTNFVNPKRPFWPWKGLEIISRRTQRRLHRIKESWGLQDLVNDFG
NLQLGPPMS
(italic: nucleic acid editing domain)
Rhesus macaque APOBEC-3G:
(SEQ ID NO: 269)
MVEPMDPRTFVSNFNNRPILSGLNTVWLCCEVKTKDPSGPPLDAKIFQGKVYSKAKYHP
EMRFLRWFHKWRQLHHDQEYKVTWYVSWSPCTRCANSVATFLAKDPKVTLTIFVARLYY
FWKPDYQQALRILCQKRGGPHATMKIMNYNEFQDCWNKFVDGRGKPFKPRNNLPKHY
TLLQATLGELLRHLMDPGTFTSNFNNKPWVSGQHETYLCYKVERLHNDTWVPLNQHR
GFLRNQAPNIHGFPKGRHAELCFLDLIPFWKLDGQQYRVTCFTSWSPCFSCAQEMAKFISN
NEHVSLCIFAARIYDDQGRYQEGLRALHRDGAKIAMMNYSEFEYCWDTFVDRQGRPFQ
PWDGLDEHSQALSGRLRAI
(italic: nucleic acid editing domain; underline: cytoplasmic
localization signal)
Chimpanzee APOBEC-3G:
(SEQ ID NO: 270)
MKPHFRNPVERMYQDTESDNFYNRPILSHRNTVWLCYEVKTKGPSRPPLDAKIFRGQVY
SKLKYHPEMRFFHWFSKWRKLHRDQEYEVTWYISWSPCTKCTRDVATFLAEDPKVTLTIF
VARLYYFWDPDYQEALRSLCQKRDGPRATMKIMNYDEFQHCWSKFVYSQRELFEPWN
NLPKYYILLHIMLGEILRHSMDPPTFTSNFNNELWVRGRHETYLCYEVERLHNDTWVLL
NQRRGFLCNQAPHKHGFLEGRHAELCFLDVIPFWKLDLHQDYRVTCFTSWSPCFSCAQEM
AKFISNNKHVSLCIFAARIYDDQGRCQEGLRTLAKAGAKISIMTYSEFKHCWDTFVDHQ
GCPFQPWDGLEEHSQALSGRLRAILQNQGN
(italic: nucleic acid editing domain; underline: cytoplasmic
localization signal)
Green monkey APOBEC-3G:
(SEQ ID NO: 271)
MNPQIRNMVEQMEPDIFVYYFNNRPILSGRNTVWLCYEVKTKDPSGPPLDANIFQGKLY
PEAKDHPEMKFLHWFRKWRQLHRDQEYEVTWYVSWSPCTRCANSVATFLAEDPKVTLTIF
VARLYYFWKPDYQQALRILCQERGGPHATMKIMNYNEFQHCWNEFVDGQGKPFKPRK
NLPKHYTLLHATLGELLRHVMDPGTFTSNFNNKPWVSGQRETYLCYKVERSHNDTWV
LLNQHRGFLRNQAPDRHGFPKGRHAELCFLDLIPFWKLDDQQYRVTCFTSWSPCFSCAQK
MAKFISNNKHVSLCIFAARIYDDQGRCQEGLRTLHRDGAKIAVMNYSEFEYCWDTFVD
RQGRPFQPWDGLDEHSQALSGRLRAI
(italic: nucleic acid editing domain; underline: cytoplasmic
localization signal)
Human APOBEC-3G:
(SEQ ID NO: 272)
MKPHFRNTVERMYRDTFSYNFYNRPILSRRNTVWLCYEVKTKGPSRPPLDAKIFRGQVY
SELKYHPEMRFFHWFSKWRKLHRDQEYEVTWYISWSPCTKCTRDMATFLAEDPKVTLTIF
VARLYYFWDPDYQEALRSLCQKRDGPRATMKIMNYDEFQHCWSKFVYSQRELFEPWN
NLPKYYILLHIMLGEILRHSMDPPTFTFNFNNEPWVRGRHETYLCYEVERMHNDTWVLL
NQRRGFLCNQAPHKHGFLEGRHAELCFLDVIPFWKLDLDQDYRVTCFTSWSPCFSCAQEM
AKFISKNKHVSLCIFTARIYDDQGRCQEGLRTLAEAGAKISIMTYSEFKHCWDTFVDHQ
GCPFQPWDGLDEHSQDLSGRLRAILQNQEN
(italic: nucleic acid editing domain; underline: cytoplasmic
localization signal)
Human APOBEC-3F:
(SEQ ID NO: 273)
MKPHFRNTVERMYRDTFSYNFYNRPILSRRNTVWLCYEVKTKGPSRPRLDAKIFRGQV
YSQPEHHAEMCFLSWFCGNQLPAYKCFQITWFVSWTPCPDCVAKLAEFLAEHPNVTLTIS
AARLYYYWERDYRRALCRLSQAGARVKIMDDEEFAYCWENFVYSEGQPFMPWYKFD
DNYAFLHRTLKEILRNPMEAMYPHIFYFHFKNLRKAYGRNESWLCFTMEVVKHHSPVS
WKRGVFRNQVDPETHCHAERCFLSWFCDDILSPNTNYEVTWYTSWSPCPECAGEVAEFLA
RHSNVNLTIFTARLYYFWDTDYQEGLRSLSQEGASVEIMGYKDFKYCWENFVYNDDEP
FKPWKGLKYNFLFLDSKLQEILE
(italic: nucleic acid editing domain)
Human APOBEC-3B:
(SEQ ID NO: 274)
MNPQIRNPMERMYRDTFYDNFENEPILYGRSYTWLCYEVKIKRGRSNLLWDTGVFRGQ
VYFKPQYHAEMCFLSWFCGNQLPAYKCFQITWFVSWTPCPDCVAKLAEFLSEHPNVTLTI
SAARLYYYWERDYRRALCRLSQAGARVTIMDYEEFAYCWENFVYNEGQQFMPWYKF
DENYAFLHRTLKEILRYLMDPDTFTFNFNNDPLVLRRRQTYLCYEVERLDNGTWVLMD
QHMGFLCNEAKNLLCGFYGRHAELRFLDLVPSLQLDPAQIYRVTWFISWSPCFSWGCAGE
VRAFLQENTHVRLRIFAARIYDYDPLYKEALQMLRDAGAQVSIMTYDEFEYCWDTFVY
RQGCPFQPWDGLEEHSQALSGRLRAILQNQGN
(italic: nucleic acid editing domain)
Human APOBEC-3C:
(SEQ ID NO: 275)
MNPQIRNPMKAMYPGTFYFQFKNLWEANDRNETWLCFTVEGIKRRSVVSWKTGVFRN
QVDSETHCHAERCFLSWFCDDILSPNTKYQVTWYTSWSPCPDCAGEVAEFLARHSNVNLTI
FTARLYYFQYPCYQEGLRSLSQEGVAVEIMDYEDFKYCWENFVYNDNEPFKPWKGLKT
NFRLLKRRLRESLQ
(italic: nucleic acid editing domain)
Human APOBEC-3A:
(SEQ ID NO: 276)
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQENTH
VRLRIFAARIYDYDPLYKEALQMLRDAGAQVSIMTYDEFKHCWDTFVDHQGCPFQPWD
GLDEHSQALSGRLRAILQNQGN
(italic: nucleic acid editing domain)
Human APOBEC-3H:
(SEQ ID NO: 277)
MALLTAETFRLQFNNKRRLRRPYYPRKALLCYQLTPQNGSTPTRGYFENKKKCHAEICFI
NEIKSMGLDETQCYQVTCYLTWSPCSSCAWELVDFIKAHDHLNLGIFASRLYYHWCKPQQ
KGLRLLCGSQVPVEVMGFPKFADCWENFVDHEKPLSFNPYKMLEELDKNSRAIKRRLE
RIKIPGVRAQGRYMDILCDAEV
(italic: nucleic acid editing domain)
Human APOBEC-3D:
(SEQ ID NO: 278)
MNPQIRNPMERMYRDTFYDNFENEPILYGRSYTWLCYEVKIKRGRSNLLWDTGVFRGP
VLPKRQSNHRQEVYFRFENHAEMCFLSWFCGNRLPANRRFQITWFVSWNPCLPCVVKVT
KFLAEHPNVTLTISAARLYYYRDRDWRWVLLRLHKAGARVKIMDYEDFAYCWENFVC
NEGQPFMPWYKFDDNYASLHRTLKEILRNPMEAMYPHIFYFHFKNLLKACGRNESWLC
FTMEVTKHHSAVFRKRGVFRNQVDPETHCHAERCFLSWFCDDILSPNTNYEVTWYTSWSP
CPECAGEVAEFLARHSNVNLTIFTARLCYFWDTDYQEGLCSLSQEGASVKIMGYKDFVS
CWKNFVYSDDEPFKPWKGLQTNFRLLKRRLREILQ
(italic: nucleic acid editing domain)
Human APOBEC-1:
(SEQ ID NO: 279)
MTSEKGPSTGDPTLRRRIEPWEFDVFYDPRELRKEACLLYEIKWGMSRKIWRSSGKNTT
NHVEVNFIKKFTSERDFHPSMSCSITWFLSWSPCWECSQAIREFLSRHPGVTLVIYVARLF
WHMDQQNRQGLRDLVNSGVTIQIMRASEYYHCWRNFVNYPPGDEAHWPQYPPLWMM
LYALELHCIILSLPPCLKISRRWQNHLTFFRLHLQNCHYQTIPPHILLATGLIHPSVAWR
Mouse APOBEC-1:
(SEQ ID NO: 280)
MSSETGPVAVDPTLRRRIEPHEFEVFFDPRELRKETCLLYEINWGGRHSVWRHTSQNTSN
HVEVNFLEKFTTERYFRPNTRCSITWFLSWSPCGECSRAITEFLSRHPYVTLFIYIARLYH
HTDQRNRQGLRDLISSGVTIQIMTEQEYCYCWRNFVNYPPSNEAYWPRYPHLWVKLYV
LELYCIILGLPPCLKILRRKQPQLTFFTITLQTCHYQRIPPHLLWATGLK
Rat APOBEC-1:
(SEQ ID NO: 281)
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHH
ADPRNRQGLRDLISSGVTIQIMTEQESGYCWRNFVNYSPSNEAHWPRYPHLWVRLYVL
ELYCIILGLPPCLNILRRKQPQLTFFTIALQSCHYQRLPPHILWATGLK

In some embodiments, fusion proteins as provided herein comprise the full-length amino acid of a effector domain, e.g., one of the sequences provided above. In other embodiments, however, fusion proteins as provided herein do not comprise a full-length sequence of a effector domain, but only a fragment thereof. For example, in some embodiments, a fusion protein provided herein comprises a Cas9 protein and a fragment of a effector domain, e.g., wherein the fragment comprises a effector domain. Exemplary amino acid sequences of effector domains are shown in the sequences above as italicized letters, and additional suitable sequences of such domains will be apparent to those of skill in the art.

Additional suitable nucleic-acid editing domain, e.g., deaminase domain sequences, that can be used according to aspects of this disclosure, e.g., that can be fused to a nuclease-inactive Cas9 protein, will be apparent to those of skill in the art based on this disclosure. In some embodiments, such additional domain sequences include deaminase domain sequences that are at least 70%, at least 75%, at least 80%, at least 85%, at least 90%, at least 95%, at least 96%, at least 97%, at least 98%, or at least 99% similar to the sequences provided herein. Additional suitable Cas9 proteins, variants, and sequences will also be apparent to those of skill in the art. Examples of such additional suitable Cas9 proteins include, but are not limited to Cas9 proteins with the following mutations: D10A, D10A/D839A/H840A, and D10A/D839A/H840A/N863A (See, e.g., Prashant et al., CAS9 transcriptional activators for target specificity screening and paired nickases for cooperative genome engineering. Nature Biotechnology. 2013; 31 (9): 833-838 the entire contents of which are incorporated herein by reference).

Additional suitable strategies for generating fusion proteins comprising a Cas9 protein and an effector domain, such as a DNA-editing domain, will be apparent to those of skill in the art based on this disclosure in combination with the general knowledge in the art. Suitable strategies for generating fusion proteins according to aspects of this disclosure using linkers or without the use of linkers will also be apparent to those of skill in the art in view of the instant disclosure and the knowledge in the art. For example, Gilbert et al., CRISPR-mediated modular RNA-guided regulation of transcription in eukaryotes. Cell. 2013; 154 (2): 442-51, showed that C-terminal fusions of Cas9 with VP64 using 2 NLS's as a linker (SPKKKRKVEAS, SEQ ID NO: 284), can be employed for transcriptional activation. Mali et al., Cas9 transcriptional activators for target specificity screening and paired nickases for cooperative genome engineering. Nat Biotechnol. 2013; 31 (9): 833-8, reported that C-terminal fusions with VP64 without linker can be employed for transcriptional activation. And Maeder et al., CRISPR RNA-guided activation of endogenous human genes. Nat Methods. 2013; 10:977-979, reported that C-terminal fusions with VP64 using a Gly4Ser (SEQ ID NO: 5) linker can be used as transcriptional activators. Recently, dCas9-FokI nuclease fusions have successfully been generated and exhibit improved enzymatic specificity as compared to the parental Cas9 enzyme (In Guilinger J P, Thompson D B, Liu D R. Fusion of catalytically inactive Cas9 to FokI nuclease improves the specificity of genome modification. Nat. Biotechnol. 2014; 32 (6): 577-82, and in Tsai S Q, Wyvekens N, Khayter C, Foden J A, Thapar V, Reyon D, Goodwin M J, Aryee M J, Joung J K. Dimeric CRISPR RNA-guided FokI nucleases for highly specific genome editing. Nat. Biotechnol. 2014; 32 (6): 569-76. PMID: 24770325 a SGSETPGTSESATPES (SEQ ID NO: 7) or a GGGGS (SEQ ID NO: 5) linker was used in FokI-dCas9 fusion proteins, respectively).

In some embodiments, the Cas9 fusion protein comprises: (i) Cas9 protein; and (ii) a transcriptional activator domain. In some embodiments, the transcriptional activator domain comprises a VPR. VPR is a VP64-SV40-P65-RTA tripartite activator. In some embodiments, VPR comprises a VP64 amino acid sequence encoded by the nucleic acid sequence of SEQ ID NO: 292:

(SEQ ID NO: 292)
GAGGCCAGCGGTTCCGGACGGGCTGACGCATTGGACGATTTTGATCTGG
ATATGCTGGGAAGTGACGCCCTCGATGATTTTGACCTTGACATGCTTGG
TTCGGATGCCCTTGATGACTTTGACCTCGACATGCTCGGCAGTGACGCC
CTTGATGATTTCGACCTGGACATGCTGATTAACTCTAGATAG

In some embodiments. VPR comprises a VP64 amino acid sequence as set forth in SEQ ID NO: 293:

(SEQ ID NO: 293)
EASGSGRADALDDFDLDMLGSDALDDFDLDMLGSDALDDFDLDMLGSDAL
DDFDLDMLINSR

In some embodiments. VPR comprises a VP64-SV40-P65-RTA amino acid sequence encoded by the nucleic acid sequence of SEQ ID NO: 294:

(SEQ ID NO: 294)
TCGCCAGGGATCCGTCGACTTGACGCGTTGATATCAACAAGTTTGTACAAAAAAGCA
GGCTACAAAGAGGCCAGCGGTTCCGGACGGGCTGACGCATTGGACGATTTTGATCT
GGATATGCTGGGAAGTGACGCCCTCGATGATTTTGACCTTGACATGCTTGGTTCGGA
TGCCCTTGATGACTTTGACCTCGACATGCTCGGCAGTGACGCCCTTGATGATTTCGA
CCTGGACATGCTGATTAACTCTAGAAGTTCCGGATCTCCGAAAAAGAAACGCAAAG
TTGGTAGCCAGTACCTGCCCGACACCGACGACCGGCACCGGATCGAGGAAAAGCGG
AAGCGGACCTACGAGACATTCAAGAGCATCATGAAGAAGTCCCCCTTCAGCGGCCC
CACCGACCCTAGACCTCCACCTAGAAGAATCGCCGTGCCCAGCAGATCCAGCGCCA
GCGTGCCAAAACCTGCCCCCCAGCCTTACCCCTTCACCAGCAGCCTGAGCACCATCA
ACTACGACGAGTTCCCTACCATGGTGTTCCCCAGCGGCCAGATCTCTCAGGCCTCTG
CTCTGGCTCCAGCCCCTCCTCAGGTGCTGCCTCAGGCTCCTGCTCCTGCACCAGCTCC
AGCCATGGTGTCTGCACTGGCTCAGGCACCAGCACCCGTGCCTGTGCTGGCTCCTGG
ACCTCCACAGGCTGTGGCTCCACCAGCCCCTAAACCTACACAGGCCGGCGAGGGCA
CACTGTCTGAAGCTCTGCTGCAGCTGCAGTTCGACGACGAGGATCTGGGAGCCCTGC
TGGGAAACAGCACCGATCCTGCCGTGTTCACCGACCTGGCCAGCGTGGACAACAGC
GAGTTCCAGCAGCTGCTGAACCAGGGCATCCCTGTGGCCCCTCACACCACCGAGCCC
ATGCTGATGGAATACCCCGAGGCCATCACCCGGCTCGTGACAGGCGCTCAGAGGCC
TCCTGATCCAGCTCCTGCCCCTCTGGGAGCACCAGGCCTGCCTAATGGACTGCTGTC
TGGCGACGAGGACTTCAGCTCTATCGCCGATATGGATTTCTCAGCCTTGCTGGGCTC
TGGCAGCGGCAGCCGGGATTCCAGGGAAGGGATGTTTTTGCCGAAGCCTGAGGCCG
GCTCCGCTATTAGTGACGTGTTTGAGGGCCGCGAGGTGTGCCAGCCAAAACGAATCC
GGCCATTTCATCCTCCAGGAAGTCCATGGGCCAACCGCCCACTCCCCGCCAGCCTCG
CACCAACACCAACCGGTCCAGTACATGAGCCAGTCGGGTCACTGACCCCGGCACCA
GTCCCTCAGCCACTGGATCCAGCGCCCGCAGTGACTCCCGAGGCCAGTCACCTGTTG
GAGGATCCCGATGAAGAGACGAGCCAGGCTGTCAAAGCCCTTCGGGAGATGGCCGA
TACTGTGATTCCCCAGAAGGAAGAGGCTGCAATCTGTGGCCAAATGGACCTTTCCCA
TCCGCCCCCAAGGGGCCATCTGGATGAGCTGACAACCACACTTGAGTCCATGACCG
AGGATCTGAACCTGGACTCACCCCTGACCCCGGAATTGAACGAGATTCTGGATACCT
TCCTGAACGACGAGTGCCTCTTGCATGCCATGCATATCAGCACAGGACTGTCCATCT
TCGACACATCTCTGTTTTGA

In some embodiments, VPR comprises a VP64-SV40-P65-RTA amino acid sequence as set forth in SEQ ID NO: 295:

(SEQ ID NO: 295)
SPGIRRLDALISTSLYKKAGYKEASGSGRADALDDFDLDMLGSDALDDFD
LDMLGSDALDDFDLDMLGSDALDDFDLDMLINSRSSGSPKKKRKVGSQYL
PDTDDRHRIEEKRKRTYETFKSIMKKSPFSGPTDPRPPPRRIAVPSRSSA
SVPKPAPQPYPFTSSLSTINYDEFPTMVFPSGQISQASALAPAPPQVLPQ
APAPAPAPAMVSALAQAPAPVPVLAPGPPQAVAPPAPKPTQAGEGTLSEA
LLQLQFDDEDLGALLGNSTDPAVFTDLASVDNSEFQQLLNQGIPVAPHTT
EPMLMEYPEAITRLVTGAQRPPDPAPAPLGAPGLPNGLLSGDEDFSSIAD
MDFSALLGSGSGSRDSREGMFLPKPEAGSAISDVFEGREVCQPKRIRPFH
PPGSPWANRPLPASLAPTPTGPVHEPVGSLTPAPVPQPLDPAPAVTPEAS
HLLEDPDEETSQAVKALREMADTVIPQKEEAAICGQMDLSHPPPRGHLDE
LTTTLESMTEDLNLDSPLTPELNEILDTFLNDECLLHAMHISTGLSIFDT
SLF

Some aspects of this disclosure provide fusion proteins comprising a transcription activator. In some embodiments, the transcriptional activator is VPR. In some embodiments, the VPR comprises a wild type VPR or a VPR as set forth in SEQ ID NO: 293. In some embodiments, the VPR proteins provided herein include fragments of VPR and proteins homologous to a VPR or a VPR fragment. For example, in some embodiments, a VPR comprises a fragment of the amino acid sequence set forth in SEQ ID NO: 293. In some embodiments, a VPR comprises an amino acid sequence homologous to the amino acid sequence set forth in SEQ ID NO: 293 or an amino acid sequence homologous to a fragment of the amino acid sequence set forth in SEQ ID NO: 293. In some embodiments, proteins comprising VPR or fragments of VPR or homologs of VPR or VPR fragments are referred to as “VPR variants.” A VPR variant shares homology to VPR, or a fragment thereof. For example a VPR variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild type VPR or a VPR as set forth in SEQ ID NO: 293. In some embodiments, the VPR variant comprises a fragment of VPR, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type VPR or a VPR as set forth in SEQ ID NO: 293. In some embodiments, the VPR comprises the amino acid sequence set forth in SEQ ID NO: 293. In some embodiments, the VPR comprises an amino acid sequence encoded by the nucleotide sequence set forth in SEQ ID NO: 292.

In some embodiments, a VPR is a VP64-SV40-P65-RTA triple activator. In some embodiments, the VP64-SV40-P65-RTA comprises a VP64-SV40-P65-RTA as set forth in SEQ ID NO: 295. In some embodiments, the VP64-SV40-P65-RTA proteins provided herein include fragments of VP64-SV40-P65-RTA and proteins homologous to a VP64-SV40-P65-RTA or a VP64-SV40-P65-RTA fragment. For example, in some embodiments, a VP64-SV40-P65-RTA comprises a fragment of the amino acid sequence set forth in SEQ ID NO: 295. In some embodiments, a VP64-SV40-P65-RTA comprises an amino acid sequence homologous to the amino acid sequence set forth in SEQ ID NO: 295 or an amino acid sequence homologous to a fragment of the amino acid sequence set forth in SEQ ID NO: 295. In some embodiments, proteins comprising VP64-SV40-P65-RTA or fragments of VP64-SV40-P65-RTA or homologs of VP64-SV40-P65-RTA or VP64-SV40-P65-RTA fragments are referred to as “VP64-SV40-P65-RTA variants.” A VP64-SV40-P65-RTA variant shares homology to VP64-SV40-P65-RTA, or a fragment thereof. For example a VP64-SV40-P65-RTA variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a VP64-SV40-P65-RTA as set forth in SEQ ID NO: 295. In some embodiments, the VP64-SV40-P65-RTA variant comprises a fragment of VP64-SV40-P65-RTA, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of a VP64-SV40-P65-RTA as set forth in SEQ ID NO: 295. In some embodiments, the VP64-SV40-P65-RTA comprises the amino acid sequence set forth in SEQ ID NO: 295. In some embodiments, the VP64-SV40-P65-RTA comprises an amino acid sequence encoded by the nucleotide sequence set forth in SEQ ID NO: 294.

Some aspects of this disclosure provide fusion proteins comprising (i) a Cas9 protein; and (ii) a effector domain. In some aspects, the fusion proteins provided herein further include (iii) a DNA-binding protein, for example, a zinc-finger domain, a TALE, or a second Cas9 protein. Without wishing to be bound by any particular theory, fusing a DNA-binding protein (e.g., a second Cas9 protein) to a fusion protein comprising (i) a protein; and (ii) a effector domain may be useful for improving specificity of the fusion protein to a target nucleic acid sequence, or for improving specificity or binding affinity of the fusion protein to bind a target nucleic acid sequence that does not contain the canonical PAM (5′-NGG-3′) sequence. In some embodiments, the second Cas9 protein is any of the Cas9 proteins provided herein. In some embodiments, the second Cas9 protein is fused to the fusion protein N-terminal to the Cas9 protein. In some embodiments, the second Cas9 protein is fused to the fusion protein C-terminal to the Cas9 protein. In some embodiments, the Cas9 protein and the second Cas9 protein are fused via a linker.

Further provided herein are complexes comprising any of the fusion proteins provided herein, a first guide RNA bound to the Cas9 protein of the fusion protein, and a second guide RNA bound to the second Cas9 protein of the fusion protein. In some embodiments, the first guide RNA is about 15-100 nucleotides long and comprises a sequence of at least 10 contiguous nucleotides that is complementary to a first target sequence and the second guide RNA is about 15-100 nucleotides long and comprises a sequence of at least 10 contiguous nucleotides that is complementary to a second target sequence. In some embodiments, the first guide RNA and/or the second guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the first guide RNA and the second guide RNA are different. In some embodiments, the first guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a first target sequence, and wherein the second guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a second target sequence. In some embodiments, the first target sequence and the second target sequence are different. In some embodiments, the first target sequence and the second target sequence are DNA sequences. In some embodiments, the first target sequence and the second target sequence are in the genome of a mammal. In some embodiments, the first target sequence and the second target sequence are in the genome of a human. In some embodiments, the first target sequence is within 30 nucleotides of the second target sequence. In some embodiments, the 3′ end of the first target sequence is not immediately adjacent to the canonical PAM sequence (5′-NGG-3′). In some embodiments, the 3′ end of the second target sequence is not immediately adjacent to the canonical PAM sequence (5′-NGG-3′).

In some embodiments, the general architecture of exemplary Cas9 fusion proteins provided herein is of the structure:
[NH₂]-[effector domain]-[Cas9]-[second Cas9 protein]-[COOH];
[NH₂]-[second Cas9 protein]-[Cas9]-[effector domain]-[COOH];
[NH₂]-[Cas9]-[effector domain]-[second Cas9 protein]-[COOH];
[NH₂]-[second Cas9 protein]-[effector or domain]-[Cas9]-[COOH];
[NH₂]-[UGI]-[effector domain]-[Cas9]-[second Cas9 protein]-[COOH];
[NH₂]-[UGI]-[second Cas9 protein]-[Cas9]-[effector domain]-[COOH];
[NH₂]-[UGI]-[Cas9]-[effector domain]-[second Cas9 protein]-[COOH];
[NH₂]-[UGI]-[second Cas9 protein]-[effector domain]-[Cas9]-[COOH];
[NH₂]-[effector domain]-[Cas9]-[second Cas9 protein]-[UGI]-[COOH];
[NH₂]-[second Cas9 protein]-[Cas9]-[effector domain]-[UGI]-[COOH];
[NH₂]-[Cas9]-[effector domain]-[second Cas9 protein]-[UGI]-[COOH]; or
[NH₂]-[second Cas9 protein]-[effector domain]-[Cas9]-[UGI]-[COOH];
   wherein NH₂ is the N-terminus of the fusion protein, and COOH is the C-terminus of the fusion protein. In some embodiments, the “]-[” used in the general architecture above indicates the presence of an optional linker sequence. In other examples, the general architecture of exemplary Cas9 fusion proteins provided herein is of the structure:
[NH₂]-[effector domain]-[Cas9]-[second Cas9 protein]-[COOH];
[NH₂]-[second Cas9 protein]-[Cas9]-[effector domain]-[COOH];
[NH₂]-[Cas9]-[effector domain]-[second Cas9 protein]-[COOH];
[NH₂]-[second Cas9 protein]-[effector domain]-[Cas9]-[COOH];
[NH₂]-[UGI]-[effector domain]-[Cas9]-[second Cas9 protein]-[COOH],
[NH₂]-[UGI]-[second Cas9 protein]-[Cas9]-[effector domain]-[COOH];
[NH₂]-[UGI]-[Cas9]-[effector domain]-[second Cas9 protein]-[COOH];
[NH₂]-[UGI]-[second Cas9 protein]-[effector domain]-[Cas9]-[COOH];
[NH₂]-[effector domain]-[Cas9]-[second Cas9 protein]-[UGI]-[COOH];
[NH₂]-[second Cas9 protein]-[Cas9]-[effector domain]-[UGI]-[COOH];
[NH₂]-[Cas9]-[effector domain]-[second Cas9 protein]-[UGI]-[COOH]; or
[NH₂]-[second Cas9 protein]-[effector domain]-[Cas9]-[UGI]-[COOH];
   wherein NH₂ is the N-terminus of the fusion protein, and COOH is the C-terminus of the fusion protein. In some embodiments, the “-” used in the general architecture above indicates the presence of an optional linker sequence. In some embodiments, the second Cas9 is a dCas9 protein. In some examples, the general architecture of exemplary Cas9 fusion proteins provided herein comprises a structure as shown in FIG. 8. It should be appreciated that any of the proteins provided in any of the general architectures of exemplary Cas9 fusion proteins may be connected by one or more of the linkers provided herein. In some embodiments, the linkers are the same. In some embodiments, the linkers are different. In some embodiments, one or more of the proteins provided in any of the general architectures of exemplary Cas9 fusion proteins are not fused via a linker. In some embodiments, the fusion proteins further comprise a nuclear targeting sequence, for example a nuclear localization sequence. In some embodiments, fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the second Cas9 protein. In some embodiments, the NLS is fused to the C-terminus of the second Cas9 protein. In some embodiments, the NLS is fused to the N-terminus of the Cas9 protein. In some embodiments, the NLS is fused to the C-terminus of the Cas9 protein. In some embodiments, the NLS is fused to the N-terminus of the effector domain. In some embodiments, the NLS is fused to the C-terminus of the effector domain. In some embodiments, the NLS is fused to the N-terminus of the UGI protein. In some embodiments, the NLS is fused to the C-terminus of the UGI protein. In some embodiments, the NLS is fused to the fusion protein via one or more linkers. In some embodiments, the NLS is fused to the fusion protein without a linker.

### Uracil Glycosylase Inhibitor Fusion Proteins

Some aspects of this disclosure provide fusion proteins comprising a Cas9 protein fused to a effector domain, e.g., a deaminase, and a uracil glycosylase inhibitor (UGI). In some embodiments, the fusion protein comprises the structure:
[deaminase]-[optional linker sequence]-[Cas9]-[optional linker sequence]-[UGI];
[deaminase]-[optional linker sequence]-[UGI]-[optional linker sequence]-[Cas9];
[UGI]-[optional linker sequence]-[deaminase]-[optional linker sequence]-[Cas9];
[UGI]-[optional linker sequence]-[Cas9]-[optional linker sequence]-[deaminase];
[Cas9]-[optional linker sequence]-[deaminase]-[optional linker sequence]-[UGI]; or
[Cas9]-[optional linker sequence]-[UGI]-[optional linker sequence]-[deaminase].
   In some embodiments, the fusion proteins do not comprise a linker sequence. In some embodiments, one or both of the optional linker sequences are present.

In some embodiments, the fusion protein further comprises a second Cas9 protein. For example, the second Cas9 protein may be any of the Cas9 proteins provided herein. In some embodiments, fusion protein comprises the structure:
[deaminase]-[Cas9]-[UGI]; [deaminase]-[UGI]-[Cas9];
[UGI]-[deaminase]-[Cas9];
[UGI]-[Cas9]-[deaminase];
[Cas9]-[deaminase]-[UGI];
[Cas9]-[UGI]-[deaminase];
[second Cas9]-[deaminase]-[Cas9]-[UGI];
[second Cas9]-[deaminase]-[UGI]-[Cas9];
[second Cas9]-[UGI]-[deaminase]-[Cas9];
[second Cas9]-[UGI]-[Cas9]-[deaminase];
[second Cas9]-[Cas9]-[deaminase]-[UGI];
[second Cas9]-[Cas9]-[UGI]-[deaminase];
[deaminase]-[second Cas9]-[Cas9]-[UGI];
[deaminase]-[second Cas9]-[UGI]-[Cas9];
[UGI]-[second Cas9]-[deaminase]-[Cas9];
[UGI]-[second Cas9]-[Cas9]-[deaminase];
[Cas9]-[second Cas9]-[deaminase]-[UGI];
[Cas9]-[second Cas9]-[UGI]-[deaminase];
[deaminase]-[Cas9]-[second Cas9]-[UGI];
[deaminase]-[UGI]-[second Cas9]-[Cas9];
[UGI]-[deaminase]-[second Cas9]-[Cas9];
[UGI]-[Cas9]-[second Cas9]-[deaminase];
[Cas9]-[deaminase]-[second Cas9]-[UGI];
[Cas9]-[UGI]-[second Cas9]-[deaminase];
[deaminase]-[Cas9]-[UGI]-[second Cas9];
[deaminase]-[UGI]-[Cas9]-[second Cas9];
[UGI]-[deaminase]-[Cas9]-[second Cas9];
[UGI]-[Cas9]-[deaminase]-[second Cas9];
[Cas9]-[deaminase]-[UGI]-[second Cas9]; or
[Cas9]-[UGI]-[deaminase]-[second Cas9].
   In some embodiments, the “-” used in the general architecture above indicates the presence of an optional linker sequence. In some embodiments, the fusion proteins comprising a UGI further comprise a nuclear targeting sequence, for example a nuclear localization sequence. In some embodiments, fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the UGI protein. In some embodiments, the NLS is fused to the C-terminus of the UGI protein. In some embodiments, the NLS is fused to the N-terminus of the Cas9 protein. In some embodiments, the NLS is fused to the C-terminus of the Cas9 protein. In some embodiments, the NLS is fused to the N-terminus of the deaminase. In some embodiments, the NLS is fused to the C-terminus of the deaminase. In some embodiments, the NLS is fused to the N-terminus of the second Cas9. In some embodiments, the NLS is fused to the C-terminus of the second Cas9. In some embodiments, the NLS is fused to the fusion protein via one or more linkers. In some embodiments, the NLS is fused to the fusion protein without a linker.

In some embodiments, the UGI comprises a wild type UGI or a UGI as set forth in SEQ ID NO: 553. In some embodiments, the UGI proteins provided herein include fragments of UGI and proteins homologous to a UGI or a UGI fragment. For example, in some embodiments, a UGI comprises a fragment of the amino acid sequence set forth in SEQ ID NO: 553. In some embodiments, a UGI comprises an amino acid sequence homologous to the amino acid sequence set forth in SEQ ID NO: 553 or an amino acid sequence homologous to a fragment of the amino acid sequence set forth in SEQ ID NO: 553. In some embodiments, proteins comprising UGI or fragments of UGI or homologs of UGI or UGI fragments are referred to as “UGI variants.” A UGI variant shares homology to UGI, or a fragment thereof. For example a UGI variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild type UGI or a UGI as set forth in SEQ ID NO: 553. In some embodiments, the UGI variant comprises a fragment of UGI, such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to the corresponding fragment of wild type UGI or a UGI as set forth in SEQ ID NO: 553. In some embodiments, the UGI comprises the following amino acid sequence:

>sp|P14739|UNGI_BPPB2 Uracil-DNA glycosylase
inhibitor
(SEQ ID NO: 553)
MTNLSDIIEKETGKQLVIQESILMLPEEVEEVIGNKPESDILVHTAYDES
TDENVMLLTSDAPEYKPWALVIQDSNGENKIKML

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171 (1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419 (1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887 (1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346 (1999), the entire contents of each are incorporated herein by reference.

It should be appreciated that additional proteins may be uracil glycosylase inhibitors. For example, other proteins that are capable of inhibiting (e.g., sterically blocking) a uracil-DNA glycosylase base-excision repair enzyme are within the scope of this disclosure. In some embodiments, a uracil glycosylase inhibitor is a protein that binds DNA. In some embodiments, a uracil glycosylase inhibitor is a protein that binds single-stranded DNA. For example, a uracil glycosylase inhibitor may be a Erwinia tasmaniensis single-stranded binding protein. In some embodiments, the single-stranded binding protein comprises the amino acid sequence (SEQ ID NO: 303). In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. For example, a uracil glycosylase inhibitor is a UdgX. In some embodiments, the UdgX comprises the amino acid sequence (SEQ ID NO: 304). As another example, a uracil glycosylase inhibitor is a catalytically inactive UDG. In some embodiments, a catalytically inactive UDG comprises the amino acid sequence (SEQ ID NO: 305). It should be appreciated that other uracil glycosylase inhibitors would be apparent to the skilled artisan and are within the scope of this disclosure.

Erwinia tasmaniensis SSB (themostable single-stranded DNA binding protein)
(SEQ ID NO: 303)
MASRGVNKVILVGNLGQDPEVRYMPNGGAVANITLATSESWRDKQTGETKEKTEWHR
VVLFGKLAEVAGEYLRKGSQVYIEGALQTRKWTDQAGVEKYTTEVVVNVGGTMQML
GGRSQGGGASAGGQNGGSNNGWGQPQQPQGGNQFSGGAQQQARPQQQPQQNNAPAN
NEPPIDFDDDIP
UdgX (binds to Uracil in DNA but does not excise)
(SEQ ID NO: 304)
MAGAQDFVPHTADLAELAAAAGECRGCGLYRDATQAVFGAGGRSARIMMIGEQPGDK
EDLAGLPFVGPAGRLLDRALEAADIDRDALYVTNAVKHFKFTRAAGGKRRIHKTPSRTE
VVACRPWLIAEMTSVEPDVVVLLGATAAKALLGNDFRVTQHRGEVLHVDDVPGDPAL
VATVHPSSLLRGPKEERESAFAGLVDDLRVAADVRP
UDG (catalytically inactive human UDG, binds to Uracil in DNA but does not excise)
(SEQ ID NO: 305)
MIGQKTLYSFFSPSPARKRHAPSPEPAVQGTGVAGVPEESGDAAAIPAKKAPAGQEEPGT
PPSSPLSAEQLDRIQRNKAAALLRLAARNVPVGFGESWKKHLSGEFGKPYFIKLMGFVA
EERKHYTVYPPPHQVFTWTQMCDIKDVKVVILGQEPYHGPNQAHGLCFSVQRPVPPPPS
LENIYKELSTDIEDFVHPGHGDLSGWAKQGVLLLNAVLTVRAHQANSHKERGWEQFTD
AVVSWLNQNSNGLVFLLWGSYAQKKGSAIDRKRHHVLQTAHPSPLSVYRGFFGCRHFS
KTNELLQKSGKKPIDWKEL

### High Fidelity Cas9

Some aspects of the disclosure provide high fidelity Cas9 proteins. In some embodiments, high fidelity Cas9 proteins have decreased electrostatic interactions between the Cas9 protein and a sugar-phosphate backbone of a DNA, as compared to a wild-type Cas9 domain. In some embodiments, any of the Cas9 proteins provided herein comprise one or more mutations that decrease the association between the Cas9 protein and a sugar-phosphate backbone of a DNA. In some embodiments, any of the Cas9 proteins provided herein comprise one or more mutations that decrease the association between the Cas9 protein and a sugar-phosphate backbone of a DNA by at least 5%, at least 10%, at least 15%, at least 20%, at least 25%, at least 30%, at least 35%, at least 40%, at least 45%, at least 50%, at least 60%, at least 70%, at least 80%, at least 90%, or at least 95%. In some embodiments, any of the Cas9 proteins provided herein comprise one or more of a N497X, a R661X, a Q695X, and/or a Q926X mutation of the amino acid sequence provided in SEQ ID NO: 9, or a corresponding mutation in any of the amino acid sequences provided in SEQ ID NOs: 10-262, wherein X is any amino acid. In some embodiments, any of the Cas9 proteins provided herein comprise one or more of a N497A, a R661A, a Q695A, and/or a Q926A mutation of the amino acid sequence provided in SEQ ID NO: 9, or a corresponding mutation in any of the amino acid sequences provided in SEQ ID NOs: 10-262. In some embodiments, the Cas9 protein comprises a D10A mutation of the amino acid sequence provided in SEQ ID NO: 9, or a corresponding mutation in any of the amino acid sequences provided in SEQ ID NOs: 10-262. In some embodiments, the Cas9 protein comprises the amino acid sequence as set forth in SEQ ID NO: 306. High fidelity Cas9 proteins have been described in the art and would be apparent to the skilled artisan. For example, high fidelity Cas9 proteins have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference. It should be appreciated that, based on the present disclosure and knowledge in the art, that mutations in any Cas9 protein may be generated to make high fidelity Cas9 proteins that have decreased electrostatic interactions between the Cas9 protein and a sugar-phosphate backbone of a DNA, as compared to a wild-type Cas9 domain.

Cas9 domain where mutations relative to Cas9 of SEQ ID NO: 9 are shown in bold and underlines.

(SEQ ID NO: 306)
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTAFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGALSRKLINGIRDKQSGKTILDFLKSDGFANRNFMALIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRAITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD

Cas9 Proteins with Reduced PAM Exclusivity

Some aspects of the disclosure provide Cas9 proteins that have different PAM specificities. Typically, Cas9 proteins, such as Cas9 from S. pyogenes (spCas9), require a canonical NGG PAM sequence to bind a particular nucleic acid region. This may limit the ability to of the Cas9 protein to bind to a particular nucleotide sequence within a genome. Accordingly, in some embodiments, any of the Cas proteins provided herein may be capable of binding a nucleotide sequence that does not contain a canonical (e.g., NGG) PAM sequence. For example, Cas9 proteins that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 protein is a Cas9 protein from Staphylococcus aureus (SaCas9). In some embodiments, the SaCas9 protein is a nuclease active SaCas9, a nuclease inactive SaCas9 (SaCas9d), or a SaCas9 nickase (SaCas9n). In some embodiments, the SaCas9 comprises the amino acid sequence SEQ ID NO: 307. In some embodiments, the SaCas9 comprises a N579X mutation of SEQ ID NO: 307, or a corresponding mutation in any of the amino acid sequences provided in SEQ ID NOs: 9-262, wherein X is any amino acid except for N. In some embodiments, the SaCas9 comprises a N579A mutation of SEQ ID NO: 307, or a corresponding mutation in any of the amino acid sequences provided in SEQ ID NOs: 10-262. In some embodiments, the SaCas9 protein, the SaCas9d protein, or the SaCas9n protein can bind to a nucleic acid sequence having a non-canonical PAM. In some embodiments, the SaCas9 protein, the SaCas9d protein, or the SaCas9n protein can bind to a nucleic acid sequence having a NNGRRT PAM sequence. In some embodiments, the SaCas9 protein comprises one or more of a E781X, N967X, or R1014X mutation of SEQ ID NO: 307, or a corresponding mutation in any of the amino acid sequences provided in SEQ ID NOs: 10-262, wherein X is any amino acid. In some embodiments, the SaCas9 protein comprises one or more of a E781K, N967K, or R1014H mutation of SEQ ID NO: 307, or one or more corresponding mutation in any of the amino acid sequences provided in SEQ ID NOs: 10-262. In some embodiments, the SaCas9 protein comprises a E781K, a N967K, and a R1014H mutation of SEQ ID NO: 307, or corresponding mutations in any of the amino acid sequences provided in SEQ ID NOs: 10-262. It should be appreciated that these mutations may be combined with any of the other mutations provided herein

In some embodiments, the Cas9 protein of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 307-309. In some embodiments, the Cas9 protein of any of the fusion proteins provided herein comprises the amino acid sequence of any one of SEQ ID NOs: 307-309. In some embodiments, the Cas9 protein of any of the fusion proteins provided herein consists of the amino acid sequence of any one of SEQ ID NOs: 307-309.

### Exemplary SaCas9 Sequence

(SEQ ID NO: 307)
KRNYILGLDIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMGHC
TYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEENSKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG

Residue N579 of SEQ ID NO: 307, which is underlined and in bold, may be mutated (e.g., to a A579) to yield a SaCas9 nickase.
Exemplary SaCas9n sequence

(SEQ ID NO: 308)
KRNYILGLDIGITSVGYGIIDYETRDVIDAGVRLFKEANVENNEGRRSKRGARRLKRRRR
HRIQR VKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMGHC
TYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEEASKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
ELINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYNNDLIKINGELYRVIGVNNDLLNRIEVNMIDITYREYLENMNDKRPPR
IIKTIASKTQSIKKYSTDILGNLYEVKSKKHPQIIKKG

Residue A579 of SEQ ID NO: 308, which can be mutated from N579 of SEQ ID NO: 307 to yield a SaCas9 nickase, is underlined and in bold.

### Exemplary SaKKH Cas9

(SEQ ID NO: 309)
KRNYILGLDIGITSVGYGIIDYETRDVIDAGVRLFKEANVENNEGRRSKRGARRLKRRRR
HRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKE
AKQLLKVQKAYHQLDQSFIDTYIDLLETRRTYYEGPGEGSPFGWKDIKEWYEMLMGHC
TYFPEELRSVKYAYNADLYNALNDLNNLVITRDENEKLEYYEKFQIIENVFKQKKKPTL
KQIAKEILVNEEDIKGYRVTSTGKPEFTNLKVYHDIKDITARKEIIENAELLDQIAKILTIY
QSSEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAREKN
SKDAQKMINEMQKRNRQTNERIEEIIRTTGKENAKYLIEKIKLHDMQEGKCLYSLEAIPL
EDLLNNPFNYEVDHIIPRSVSFDNSFNNKVLVKQEEASKKGNRTPFQYLSSSDSKISYETF
KKHILNLAKGKGRISKTKKEYLLEERDINRFSVQKDFINRNLVDTRYATRGLMNLLRSYF
RVNNLDVKVKSINGGFTSFLRRKWKFKKERNKGYKHHAEDALIIANADFIFKEWKKLD
KAKKVMENQMFEEKQAESMPEIETEQEYKEIFITPHQIKHIKDFKDYKYSHRVDKKPNR
KLINDTLYSTRKDDKGNTLIVNNLNGLYDKDNDKLKKLINKSPEKLLMYHHDPQTYQK
LKLIMEQYGDEKNPLYKYYEETGNYLTKYSKKDNGPVIKKIKYYGNKLNAHLDITDDY
PNSRNKVVKLSLKPYRFDVYLDNGVYKFVTVKNLDVIKKENYYEVNSKCYEEAKKLK
KISNQAEFIASFYKNDLIKINGELYRVIGVNNDLLNRIEVNMIDITYREYLENMNDKRPPH
IIKTIASKTQSIKKYSTDILGNLYEVKSKKHPQIIKKG

Residue A579 of SEQ ID NO: 309, which can be mutated from N579 of SEQ ID NO: 307 to yield a SaCas9 nickase, is underlined and in bold. Residues K781, K967, and H1014 of SEQ ID NO: 309, which can be mutated from E781, N967, and R1014 of SEQ ID NO: 307 to yield a SaKKH Cas9 are underlined and in italics.

In some embodiments, the Cas9 protein is a Cas9 protein from Streptococcus pyogenes (SpCas9). In some embodiments, the SpCas9 protein is a nuclease active SpCas9, a nuclease inactive SpCas9 (SpCas9d), or a SpCas9 nickase (SpCas9n). In some embodiments, the SpCas9 comprises the amino acid sequence SEQ ID NO: 9. In some embodiments, the SpCas9 comprises a D10X mutation of SEQ ID NO: 9, or a corresponding mutation in any of the amino acid sequences provided in SEQ ID NOs: 10-262, wherein X is any amino acid except for D. In some embodiments, the SpCas9 comprises a D10A mutation of SEQ ID NO: 9, or a corresponding mutation in any of the amino acid sequences provided in SEQ ID NOs: 10-262. In some embodiments, the SpCas9 protein, the SpCas9d protein, or the SpCas9n protein can bind to a nucleic acid sequence having a non-canonical PAM. In some embodiments, the SpCas9 protein, the SpCas9d protein, or the SpCas9n protein can bind to a nucleic acid sequence having a NGG, a NGA, or a NGCG PAM sequence. In some embodiments, the SpCas9 protein comprises one or more of a D1135X, R1335X, and T1337X mutation of SEQ ID NO: 9, or a corresponding mutation in any of the amino acid sequences provided in SEQ ID NOs: 10-262, wherein X is any amino acid. In some embodiments, the SpCas9 protein comprises one or more of a D1135E, R1335Q, and T1337R mutation of SEQ ID NO: 9, or a corresponding mutation in any of the amino acid sequences provided in SEQ ID NOs: 10-262. In some embodiments, the SpCas9 protein comprises a D1135E, a R1335Q, and a T1335R mutation of SEQ ID NO: 9, or corresponding mutations in any of the amino acid sequences provided in SEQ ID NOs: 10-262. In some embodiments, the SpCas9 protein comprises one or more of a D1135X, R1335X, and T1337X mutation of SEQ ID NO: 9, or a corresponding mutation in any of the amino acid sequences provided in SEQ ID NOs: 10-262, wherein X is any amino acid. In some embodiments, the SpCas9 protein comprises one or more of a D1135V, R1335Q, and T1337R mutation of SEQ ID NO: 9, or a corresponding mutation in any of the amino acid sequences provided in SEQ ID NOs: 10-262. In some embodiments, the SpCas9 protein comprises a D1135V, a R1335Q, and a T1337R mutation of SEQ ID NO: 9, or corresponding mutations in any of the amino acid sequences provided in SEQ ID NOs: 10-262. In some embodiments, the SpCas9 protein comprises one or more of a D1135X, G1218X, R1335X, and T1337X mutation of SEQ ID NO: 9, or a corresponding mutation in any of the amino acid sequences provided in SEQ ID NOs: 10-262, wherein X is any amino acid. In some embodiments, the SpCas9 protein comprises one or more of a D1135V, G1218R, R1335Q, and T1337R mutation of SEQ ID NO: 9, or a corresponding mutation in any of the amino acid sequences provided in SEQ ID NOs: 10-262. In some embodiments, the SpCas9 protein comprises a D1135V, a G1218R, a R1335Q, and a T1337R mutation of SEQ ID NO: 9, or corresponding mutations in any of the amino acid sequences provided in SEQ ID NOs: 10-262. It should be appreciated that these mutations may be combined with any of the other mutations provided herein

In some embodiments, the Cas9 protein of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 9, 310-313. In some embodiments, the Cas9 domain of any of the fusion proteins provided herein comprises the amino acid sequence of any one of SEQ ID NOs: 9, 310-313. In some embodiments, the Cas9 domain of any of the fusion proteins provided herein consists of the amino acid sequence of any one of SEQ ID NOs: 9, 310-313.

### Exemplary SpCas9

(SEQ ID NO: 9)
MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD

### Exemplary SpCas9n

(SEQ ID NO: 310)
DKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FDSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKRYTSTKEVLDATLIHQSITGLYETRIDLSQLGGD

### Exemplary SpEQR Cas9

(SEQ ID NO: 311)
DKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FESPTVAYSVLVVAKVEKGKSKKLKSVKELLGITIMERSSFEKNPIDFLEAKGYKEVKKD
LIIKLPKYSLFELENGRKRMLASAGELQKGNELALPSKYVNFLYLASHYEKLKGSPEDNE
QKQLFVEQHKHYLDEIIEQISEFSKRVILADANLDKVLSAYNKHRDKPIREQAENIIHLFT
LTNLGAPAAFKYFDTTIDRKQYRSTKEVLDATLIHQSITGLYETRIDLSQLGGD

Residues E1134, Q1334, and R1336 of SEQ ID NO: 311, which can be mutated from D1134. R1334, and T1336 of SEQ ID NO: 9 to yield a SpEQR Cas9, are underlined and in bold.

### Exemplary SpVQR Cas9

(SEQ ID NO: 312)
DKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FVSPTVAYSVLVVAKVEKGKSKKLKSVKELLGITIMERSSFEKNPIDFLEAKGYKEVKK
DLIIKLPKYSLFELENGRKRMLASAGELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKQYRSTKEVLDATLIHQSITGLYETRIDLSQLGGD

Residues V1134, Q1334, and R1336 of SEQ ID NO: 312, which can be mutated from D1134. R1334, and T1336 of SEQ ID NO: 9 to yield a SpVQR Cas9, are underlined and in bold.

### Exemplary SpVRER Cas9

(SEQ ID NO: 313)
DKKYSIGLAIGTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDKKHERHPIFGN
IVDEVAYHEKYPTIYHLRKKLVDSTDKADLRLIYLALAHMIKFRGHFLIEGDLNPDNSDV
DKLFIQLVQTYNQLFEENPINASGVDAKAILSARLSKSRRLENLIAQLPGEKKNGLFGNLI
ALSLGLTPNFKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLS
GEQKKAIVDLLFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRE
RMKRIEEGIKELGSQILKEHPVENTQLQNEKLYLYYLQNGRDMYVDQELDINRLSDYDV
DHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVI
TLKSKLVSDFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIETNGETGEIVWD
KGRDFATVRKVLSMPQVNIVKKTEVQTGGFSKESILPKRNSDKLIARKKDWDPKKYGG
FVSPTVAYSVLVVAKVEKGKSKKLKSVKELLGITIMERSSFEKNPIDFLEAKGYKEVKK
DLIIKLPKYSLFELENGRKRMLASARELQKGNELALPSKYVNFLYLASHYEKLKGSPED
NEQKQLFVEQHKHYLDEIIEQISEFSKRVILADANLDKVLSAYNKHRDKPIREQAENIIHL
FTLTNLGAPAAFKYFDTTIDRKEYRSTKEVLDATLIHQSITGLYETRIDLSQLGGD

Residues V1134, R1217, Q1334, and R1336 of SEQ ID NO: 313, which can be mutated from D1134, G1217, R1334, and T1336 of SEQ ID NO: 9 to yield a SpVRER Cas9, are underlined and in bold.
Cas9 Complexes with Guide RNAs

Some aspects of this disclosure provide complexes comprising a Cas9 protein or a Cas9 fusion protein as provided herein, and a guide RNA bound to the Cas9 protein or the Cas9 fusion protein. In some embodiments, the guide RNA is about 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is a sequence in the genome of a mammal. In some embodiments, the target sequence is a sequence in the genome of a human. In some embodiments, the 3′ end of the target sequence is not immediately adjacent to the canonical PAM sequence (5′-NGG-3′).

Some aspects of the disclosure provide complexes comprising a first guide RNA bound to a Cas9 protein of a fusion protein, and a second guide RNA bound to a second Cas9 protein of the fusion protein. In some embodiments, the first guide RNA is about 15-100 nucleotides long and comprises a sequence of at least 10 contiguous nucleotides that is complementary to a first target sequence and the second guide RNA is about 15-100 nucleotides long and comprises a sequence of at least 10 contiguous nucleotides that is complementary to a second target sequence. In some embodiments, the first guide RNA and/or the second guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the first guide RNA and the second guide RNA are different. In some embodiments, the first guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a first target sequence and wherein the second guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a second target sequence.

In some embodiments, first target sequence and the second target sequence are different. In some embodiments, the first target sequence and the second target sequence are DNA sequences. In some embodiments, the first target sequence and the second target sequence are in the genome of a mammal. In some embodiments, the first target sequence and the second target sequence are in the genome of a human. In some embodiments, the first target sequence is within at least 5, 10, 15, 20, 25, 30, 35, 40, 45, 50, 60, 70, 80, 90, 100, 150, or 200 nucleotides of the second target sequence. In some embodiments, the 3′-end of the first target sequence is not immediately adjacent to the canonical PAM sequence (5′-NGG-3′). In some embodiments, the 3′-end of the second target sequence is not immediately adjacent to the canonical PAM sequence (5′-NGG-3′).

### Methods of Using Cas9 Fusion Proteins

Some aspects of this disclosure provide methods of using the Cas9 proteins, fusion proteins, or complexes provided herein. For example, some aspects of this disclosure provide methods comprising contacting a DNA molecule (a) with any of the Cas9 proteins or fusion proteins provided herein, and with at least one guide RNA, wherein the guide RNA is about 15-100 nucleotides long and comprises a sequence of at least 10 contiguous nucleotides that is complementary to a target sequence; or (b) with a Cas9 protein, a Cas9 fusion protein, or a Cas9 protein or fusion protein complex with at least one gRNA as provided herein. In some embodiments, the 3′ end of the target sequence is not immediately adjacent to the canonical PAM sequence (5′-NGG-3′). In some embodiments, the 3′ end of the target sequence is immediately adjacent to an AGC, GAG, TTT, GTG, or CAA sequence.

In some embodiments, the target DNA sequence comprises a sequence associated with a disease or disorder. In some embodiments, the target DNA sequence comprises a point mutation associated with a disease or disorder. In some embodiments, the activity of the Cas9 protein, the Cas9 fusion protein, or the complex results in a correction of the point mutation. In some embodiments, the target DNA sequence comprises a T→C point mutation associated with a disease or disorder, and wherein the deamination of the mutant C base results in a sequence that is not associated with a disease or disorder. In some embodiments, the target DNA sequence encodes a protein and wherein the point mutation is in a codon and results in a change in the amino acid encoded by the mutant codon as compared to the wild-type codon. In some embodiments, the deamination of the mutant C results in a change of the amino acid encoded by the mutant codon. In some embodiments, the deamination of the mutant C results in the codon encoding the wild-type amino acid. In some embodiments, the contacting is in vivo in a subject. In some embodiments, the subject has or has been diagnosed with a disease or disorder. In some embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNB1 protein, a mutant HRAS protein, or a mutant p53 protein.

Some embodiments provide methods for using the Cas9 DNA editing fusion proteins provided herein. In some embodiments, the fusion protein is used to introduce a point mutation into a nucleic acid by deaminating a target nucleobase, e.g., a C residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a Cas9 DNA editing fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provide herein is to restore the function of a dysfunctional gene via genome editing. The Cas9 deaminase fusion proteins provided herein can be validated for gene editing-based human therapeutics in vitro, e.g., by correcting a disease-associated mutation in human cell culture. It will be understood by the skilled artisan that the fusion proteins provided herein, e.g., the fusion proteins comprising a Cas9 domain and a nucleic acid deaminase domain can be used to correct any single point T->C or A->G mutation. In the first case, deamination of the mutant C back to U corrects the mutation, and in the latter case, deamination of the C that is base-paired with the mutant G, followed by a round of replication, corrects the mutation.

An exemplary disease-relevant mutation that can be corrected by the provided fusion proteins in vitro or in vivo is the H1047R (A3140G) polymorphism in the PI3KCA protein. The phosphoinositide-3-kinase, catalytic alpha subunit (PI3KCA) protein acts to phosphorylate the 3-OH group of the inositol ring of phosphatidylinositol. The PI3KCA gene has been found to be mutated in many different carcinomas, and thus it is considered to be a potent oncogene.⁵⁰ In fact, the A3140G mutation is present in several NCI-60 cancer cell lines, such as, for example, the HCT116, SKOV3, and T47D cell lines, which are readily available from the American Type Culture Collection (ATCC).⁵¹

In some embodiments, a cell carrying a mutation to be corrected, e.g., a cell carrying a point mutation, e.g., an A3140G point mutation in exon 20 of the PI3KCA gene, resulting in a H1047R substitution in the PI3KCA protein, is contacted with an expression construct encoding a Cas9 deaminase fusion protein and an appropriately designed sgRNA targeting the fusion protein to the respective mutation site in the encoding PI3KCA gene. Control experiments can be performed where the sgRNAs are designed to target the fusion enzymes to non-C residues that are within the PI3KCA gene. Genomic DNA of the treated cells can be extracted, and the relevant sequence of the PI3KCA genes PCR amplified and sequenced to assess the activities of the fusion proteins in human cell culture.

It will be understood that the example of correcting point mutations in PI3KCA is provided for illustration purposes and is not meant to limit the instant disclosure. The skilled artisan will understand that the instantly disclosed DNA-editing fusion proteins can be used to correct other point mutations and mutations associated with other cancers and with diseases other than cancer including other proliferative diseases.

The successful correction of point mutations in disease-associated genes and alleles opens up new strategies for gene correction with applications in therapeutics and basic research. Site-specific single-base modification systems like the disclosed fusions of Cas9 and deaminase enzymes or domains also have applications in “reverse” gene therapy, where certain gene functions are purposely suppressed or abolished. In these cases, site-specifically mutating Trp (TGG), Gln (CAA and CAG), or Arg (CGA) residues to premature stop codons (TAA, TAG, TGA) can be used to abolish protein function in vitro, ex vivo, or in vivo.

The instant disclosure provides methods for the treatment of a subject diagnosed with a disease associated with or caused by a point mutation that can be corrected by a Cas9 DNA editing fusion protein provided herein. For example, in some embodiments, a method is provided that comprises administering to a subject having such a disease, e.g., a cancer associated with a PI3KCA point mutation as described above, an effective amount of a Cas9 deaminase fusion protein that corrects the point mutation or introduces a deactivating mutation into the disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

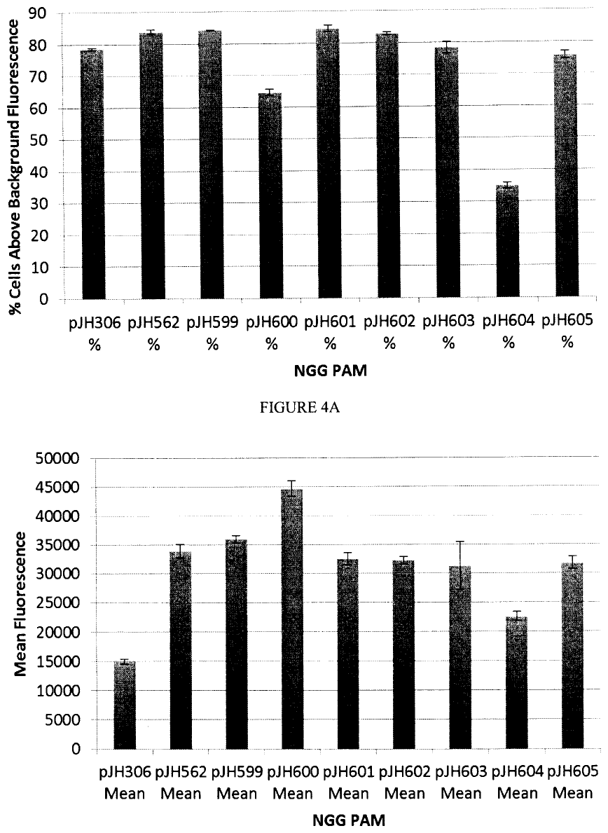

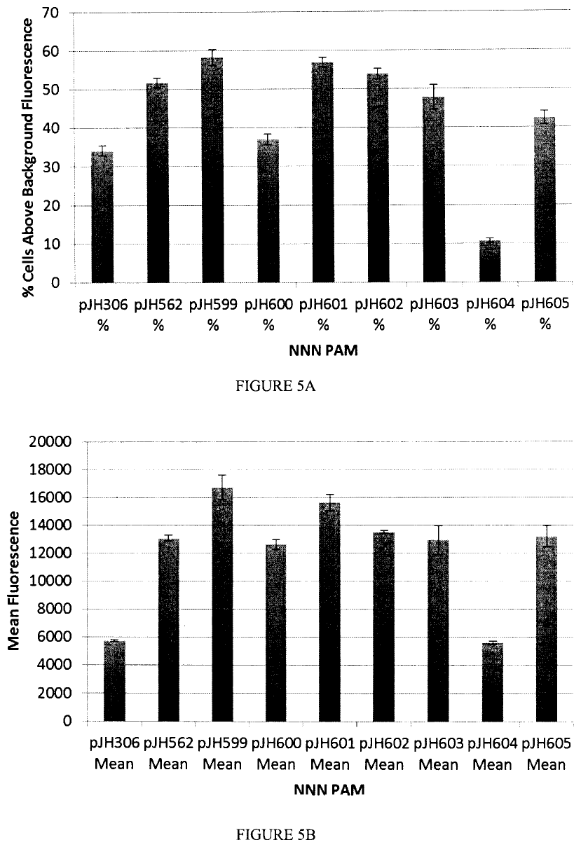

The instant disclosure provides methods for the treatment of additional diseases or disorders, e.g., diseases or disorders that are associated or caused by a point mutation that can be corrected by deaminase-mediated gene editing. Some such diseases are described herein, and additional suitable diseases that can be treated with the strategies and fusion proteins provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation, cystic fibrosis (see, e.g., Schwank et al., Functional repair of CFTR by CRISPR/Cas9 in intestinal stem cell organoids of cystic fibrosis patients. Cell stem cell. 2013; 13:653-658; and Wu et. al., Correction of a genetic disease in mouse via use of CRISPR-Cas9. Cell stem cell. 2013; 13:659-662, neither of which uses a deaminase fusion protein to correct the genetic defect); phenylketonuria—e.g., phenylalanine to serine mutation at position 835 (mouse) or 240 (human) or a homologous residue in phenylalanine hydroxylase gene (T>C mutation)—see, e.g., McDonald et al., Genomics. 1997; 39:402-405; Bernard-Soulier syndrome (BSS)—e.g., phenylalanine to serine mutation at position 55 or a homologous residue, or cysteine to arginine at residue 24 or a homologous residue in the platelet membrane glycoprotein IX (T>C mutation)—see, e.g., Noris et al., British Journal of Haematology. 1997; 97:312-320, and Ali et al., Hematol. 2014; 93:381-384; epidermolytic hyperkeratosis (EHK)—e.g., leucine to proline mutation at position 160 or 161 (if counting the initiator methionine) or a homologous residue in keratin 1 (T>C mutation)—see, e.g., Chipev et al., Cell. 1992; 70:821-828, see also accession number P04264 in the UNIPROT database at www[dot]uniprot [dot]org; chronic obstructive pulmonary disease (COPD)—e.g., leucine to proline mutation at position 54 or 55 (if counting the initiator methionine) or a homologous residue in the processed form of a1-antitrypsin or residue 78 in the unprocessed form or a homologous residue (T>C mutation)—see, e.g., Poller et al., Genomics. 1993; 17:740-743, see also accession number P01011 in the UNIPROT database; Charcot-Marie-Toot disease type 4J—e.g., isoleucine to threonine mutation at position 41 or a homologous residue in FIG. 4 (T>C mutation)—see, e.g., Lenk et al., PLOS Genetics. 2011; 7: e1002104; neuroblastoma (NB)—e.g., leucine to proline mutation at position 197 or a homologous residue in Caspase-9 (T>C mutation)—see, e.g., Kundu et al., 3 Biotech. 2013, 3:225-234; von Willebrand disease (vWD)—e.g., cysteine to arginine mutation at position 509 or a homologous residue in the processed form of von Willebrand factor, or at position 1272 or a homologous residue in the unprocessed form of von Willebrand factor (T>C mutation)—see, e.g., Lavergne et al., Br. J. Haematol. 1992, see also accession number P04275 in the UNIPROT database; 82:66-72; myotonia congenital—e.g., cysteine to arginine mutation at position 277 or a homologous residue in the muscle chloride channel gene CLCN1 (T>C mutation)—see, e.g., Weinberger et al., The J. of Physiology. 2012; 590:3449-3464; hereditary renal amyloidosis—e.g., stop codon to arginine mutation at position 78 or a homologous residue in the processed form of apolipoprotein AII or at position 101 or a homologous residue in the unprocessed form (T>C mutation)—see, e.g., Yazaki et al., Kidney Int. 2003; 64:11-16; dilated cardiomyopathy (DCM)—e.g., tryptophan to Arginine mutation at position 148 or a homologous residue in the FOXD4 gene (T>C mutation), see, e.g., Minoretti et. al., Int. J. of Mol. Med. 2007; 19:369-372; hereditary lymphedema—e.g., histidine to arginine mutation at position 1035 or a homologous residue in VEGFR3 tyrosine kinase (A>G mutation), scc, e.g., Irrthum et al., Am. J. Hum. Genet. 2000; 67:295-301; familial Alzheimer's disease—e.g., isoleucine to valine mutation at position 143 or a homologous residue in presenilin1 (A>G mutation), see, e.g., Gallo et. al., J. Alzheimer's disease. 2011; 25:425-431; Prion disease—e.g., methionine to valine mutation at position 129 or a homologous residue in prion protein (A>G mutation)—see, e.g., Lewis et. al., J. of General Virology. 2006; 87:2443-2449; chronic infantile neurologic cutaneous articular syndrome (CINCA)—e.g., Tyrosine to Cysteine mutation at position 570 or a homologous residue in cryopyrin (A>G mutation)—see, e.g., Fujisawa et. al. Blood. 2007; 109:2903-2911; and desmin-related myopathy (DRM)—e.g., arginine to glycine mutation at position 120 or a homologous residue in &B crystallin (A>G mutation)—see, e.g., Kumar et al., J. Biol. Chem. 1999; 274:24137-24141. The entire contents of all references and database entries is incorporated herein by reference.

The instant disclosure provides lists of genes comprising pathogenic T>C or A>G mutations, which may be corrected using any of the Cas9 fusion proteins provided herein. Provided herein, are the names of these genes, their respective SEQ ID NOs, their gene IDs, and sequences flanking the mutation site. See Tables 4 and 5. Without wishing to be bound by any particular theory, the mutations provided in Tables 4 and 5 may be corrected using the Cas9 fusions provided herein, which are able to bind to target sequences lacking the canonical PAM sequence. In some embodiments, a Cas9-deaminase fusion protein demonstrates activity on non-canonical PAMs and therefore can correct all the pathogenic T>C or A>G mutations listed in Tables 4 and 5 (SEQ ID NOs: 674-2539 and 3144-5083), respectively. In some embodiments, a Cas9-deaminase fusion protein recognizes canonical PAMs and therefore can correct the pathogenic T>C or A>G mutations with canonical PAMs, e.g., 5′-NGG-3′. It should be appreciated that a skilled artisan would understand how to design an RNA (e.g., a gRNA) to target any of the Cas9 proteins or fusion proteins provided herein to any target sequence in order to correct any of the mutations provided herein, for example, the mutations provided in Tables 4 and 5. It will be apparent to those of skill in the art that in order to target a Cas9:effector domain fusion protein as disclosed herein to a target site, e.g., a site comprising a point mutation to be edited, it is typically necessary to co-express the Cas9:effector domain fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9:effector domain fusion protein. In some embodiments, the guide RNA comprises a structure 5′-[guide sequence]-guuuuagagcuagaaauagcaaguuaaaauaaaggcuaguccguuaucaacuugaaaaaguggcaccgagucggugcuuuuu-3′ (SEQ ID NO: 285), wherein the guide sequence comprises a sequence that is complementary to the target sequence. The guide sequence is typically 20 nucleotides long. The sequences of suitable guide RNAs for targeting Cas9:effector domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited.

### Kits, Vectors, Cells

Some aspects of this disclosure provide kits comprising a nucleic acid construct, comprising (a) a nucleotide sequence encoding a Cas9 protein or a Cas9 fusion protein as provided herein; and (b) a heterologous promoter that drives expression of the sequence of (a). In some embodiments, the kit further comprises an expression construct encoding a guide RNA backbone, wherein the construct comprises a cloning site positioned to allow the cloning of a nucleic acid sequence identical or complementary to a target sequence into the guide RNA backbone.

Some aspects of this disclosure provide polynucleotides encoding a Cas9 protein of a fusion protein as provided herein. Some aspects of this disclosure provide vectors comprising such polynucleotides. In some embodiments, the vector comprises a heterologous promoter driving expression of polynucleotide.

Some aspects of this disclosure provide cells comprising a Cas9 protein, a fusion protein, a nucleic acid molecule, and/or a vector as provided herein.

The description of exemplary embodiments of the reporter systems herein is provided for illustration purposes only and not meant to be limiting. Additional reporter systems, e.g., variations of the exemplary systems described in detail above, are also embraced by this disclosure.

### EXAMPLES

### Example 1: PACE Evolution of a Cas9 without PAM Sequence Restriction

Building PAM Libraries. Four different protospacer target sequences were synthesized: Doench 1-5′-AAGAGAGACAGTACATGCCC-3′ (SEQ ID NO: 286);

Doench 2-5′-GGAGCCCACCGAGTACCTGG-3′ (SEQ ID NO: 287); G7′-5′-AGTCTCCTCAGCAAAACGAA-3′ (SEQ ID NO: 288); and VEGF Target 2-5′-GACCCCCTCCACCCCGCCTC-3′ (SEQ ID NO: 289). For each protospacer target sequence, a 3′-NNN PAM library was built. While the canonical PAM sequence is 5′-NGG-3′, (e.g., an exemplary [Doench 1]-[canonical PAM] target sequence could be 5′-[AAGAGAGACAGTACATGCCC]-[NGG]-3′ (SEQ ID NO: 291)), the 3′NNN PAM libraries for each protospacer target sequence contained a fully random PAM sequence, e.g., for Doench1 5′-AAGAGAGACAGTACATGCCCNNN-3′ (SEQ ID NO: 290), wherein N represents any nucleotide. The NNN PAM libraries thus included every possible combination of PAM sequences at the 3′ end of the respective protospacer target sequence.

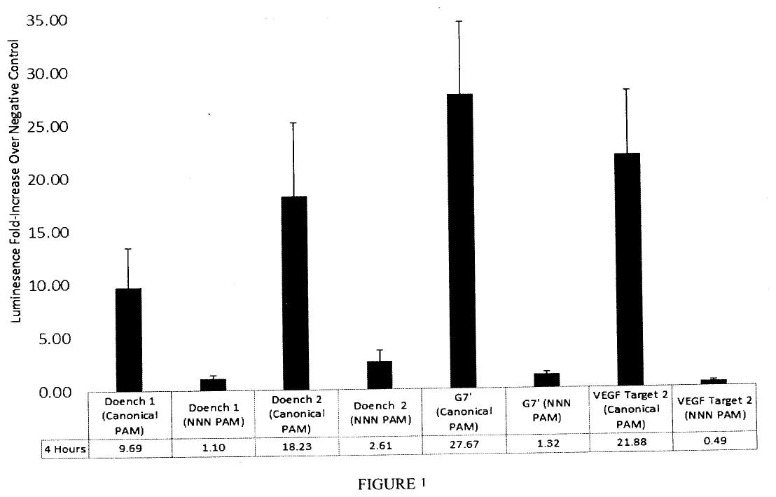

Testing the Activity of Cas9 on PAM Libraries in a ω-dCas9 luciferase assay. Cas9 activity was tested using a bacterial luciferase activation assay in which a fusion protein of the ω subunit of Escherichia coli RNA polymerase (rpoZ) to dCas9 (see, e.g., Bikard et al., Nucleic Acids Res. 2013 August; 41 (15): 7429-7437) drives the production of luciferase encoded by a nucleic acid under the control of a weak promoter comprising a sequence targeted by an sgRNA. Each PAM library was cloned into a plasmid comprising such a weak promoter, where the [target sequence]-[PAM library] nucleic acid sequences served as the sequence targeted by an sgRNA. A PAM library was cloned into the promoter for The ω-dCas9 assay was run on all four protospacer targets for both the canonical PAM and the random PAM library. FIG. 1 shows the activity of wild-type S. pyogenes Cas9 on PAM Libraries.

Evolution of Cas9 on PAM Libraries. S. pyogenes dCas9 was fused to the @ unit of RNA polymerase. The resulting ω-dCas9 fusion protein was cloned into an M13 phage-based selection phagemid (SP), comprising the entire M13 phage genome except for a functional version of the gene encoding pIII, a gene essential for the generation of infectious phage particles. The phage gene encoding pIII was provided on a separate plasmid (accessory plasmid, AP), under the control of a promoter that is transcriptionally activated by ω-dCas9. The PAM library was cloned into the promoter region of the accessory plasmid. Host cells used for the directed evolution of Cas9 proteins without PAM restriction were provided that harbored the accessory plasmid. Upon infection with a selection phage, the amount of infectious phage particles produced by a given host cell thus depends on the activity of the ω-dCas9 fusion protein encoded by the selection phage on the promoter of the accessory plasmid, which is required for the production of pIII protein. The accessory plasmid thus confers a selective advantage to those selection phages encoding ω-dCas9 fusion protein variants with an increased activity on different non-canonical PAM sequences.

A lagoon was provided and a flow of host cells comprising the accessory plasmid was generated through the lagoon. The host cells were contacted with the selection phagemid, resulting in a population of selection phage propagating in the flow of host cells in the lagoon. Phage-infected host cells were removed from the lagoon and fresh, uninfected host cells were fed into the lagoon at a rate resulting in the average time a host cell remained in the lagoon being shorter than the average time between cell division of the host cells, but longer than the average M13 phage life cycle time.

In order to generate Cas9 variants during a directed evolution experiment, the host cells in the lagoon were incubated under conditions resulting in an increased mutation rate. The host cells were harboring a mutagenesis plasmid (MP), which increased the mutagenesis rate, thus introducing mutations in the ω-dCas9 fusion protein encoded by the selection phagemid during the phage life cycle. Because the flow rate of host cells through the lagoon results in the average time a host cell remains in the lagoon being shorter than the average time between host cell divisions, the host cells in the lagoon cannot accumulate mutations resulting from the increased mutation rate conferred by the mutagenesis plasmid in their genome or on the accessory plasmid. The selection phage, however, replicate in the lagoon in the flow of host cells and thus accumulate mutations over time, resulting in the generation of new, evolved ω-dCas9 fusion protein variants.

If any of these evolved ω-dCas9 fusion protein variants includes a mutation that confers an increased activity on the accessory plasmid comprising the PAM library, this will directly translate into the generation of more pIII by a host cell infected with a selection phage encoding the respective ω-dCas9 fusion protein variant. The production of more pIII will, in turn, result in the generation of more infectious selection phage particles, which, over time, results in a competitive advantage of the mutant selection phage harboring such beneficial mutations over selection phage not harboring such mutations. After a period of time, the selective pressure exerted by the accessory plasmid will, therefore, result in selection phage having acquired beneficial mutations being the predominant species replicating in the flow of host cells, while selection phage with no mutations or with detrimental mutations will be washed out of the lagoon.

Because the ω-dCas9 fusion protein activity on the PAM library was very low at the beginning of the experiment, multiple rounds of overnight propagation of the selection phagemid in host cells harboring an accessory plasmid containing the PAM library were carried out to evolve Cas9 variants that show increased activity on noncanonical PAM sequences. At the end of a directed evolution experiment, the evolved population of selection phage was isolated from the lagoon, and a representative number of clones was analyzed to detect Cas9 variants having beneficial mutations. While all mutations observed confer a beneficial phenotype, mutations shared by more than one clone, or by all clones, are of particular interest.

Mutations from Cas9 PACE. A number of selection phage clones was isolated from a directed evolution experiment using a PAM library accessory plasmid as described above. The mutations identified in the Cas9 amino acid sequence of some exemplary clones is provided below in Table 1 (residue numbering according to SEQ ID NO: 9):

TABLE 1
Cas9 mutations identified in PACE (residue
numbering according to SEQ ID NO: 9.

| Clone | Mutations | | | | |
|---|---|---|---|---|---|
| 1 | | | D182 | | E1219V |
| 2 | | H137 | D182 | | E1219V |
| 3 | | | D182 | G660 | E1219V |
| 4 | I122 | | D182 | | E1219V |

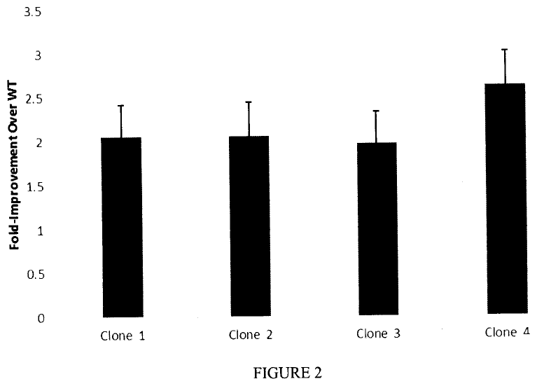

Clones 1-4 were tested in the ω-dCas9 luciferase activation assay described above. When tested on the PAM library as a whole, the different clones showed an improvement in luciferase expression (FIG. 2—Cas9 activity of exemplary evolved clones on PAM library after directed evolution).

### Improvement of Cas9 Activity on Non-Canonical PAM Sequences.

The activity of evolved Cas9 proteins on target sequences with noncanonical PAMs was evaluated in more detail. The relative activity of Clone 4, harboring 1122, D182, and E1219V mutations, for various [Doench 2 (5′-GGAGCCCACCGAGTACCTGG-3′ (SEQ ID NO: 287))]-[PAM] target sequences was tested in the ω-dCas9 luciferase activation assay and compared to the activity of wild-type dCas9.

### Improvement of Cas9 Activity on Non-Canonical PAM Sequences.

The activity of evolved Cas9 proteins on target sequences with noncanonical PAMs was evaluated in more detail. The relative activity of Clone 4, harboring 1122, D182, and E1219V mutations, for various [Doench 2 (5′-GGAGCCCACCGAGTACCTGG-3′ (SEQ ID NO: 287))]-[PAM] target sequences was tested in the ω-dCas9 luciferase activation assay and compared to the activity of wild-type dCas9. The data are shown in Table 2.

TABLE 2
Relative activity of clone 4 on various PAM sequences.

| | Clone 4 dCas9 | Wild-type dCas9 |
|---|---|---|
| PAM | Luciferase Activity | Luciferase Activity |
| GTC | 44.84 | 4.07 |
| AGC | 25.01 | 4.42 |
| GAG | 186.22 | 73.60 |
| TTT | 36.49 | 3.96 |
| GTG | 119.53 | 6.86 |
| AGG | 238.29 | 244.27 |
| CAA | 13.90 | 5.28 |

### Example 2: PACE Evolution of a Cas9 without any PAM Sequence Restriction

Because the activity of the ω-dCas9 fusion protein on the NNN-PAM libraries was very low, a second round of PACE experiments was performed, in which an initial phase of diversification of the ω-dCas9 fusion protein population was carried out in the absence of selective pressure by providing a source of pIII that did not depend on ω-dCas9 fusion protein activity. The initial diversification stage allows mutations to develop that may not be accessible in a PACE experiment where selective pressure is applied throughout the experiment.

Selection phage harboring a ω-dCas9 fusion protein with a dCas9 sequence provided as SEQ ID NO: 8 with D10A and H840A mutations was propagated overnight in 1030 host cells together with MP6 mutagenesis plasmid in the presence of arabinose in order to create a library of mutated selection phage encoding a library of ω-dCas9 fusion protein variants. PIII was expressed from a separate plasmid in the host cells during this initial diversification stage. After overnight (12 h) diversification, 1030 host cells harboring an accessory plasmid comprising an NNN PAM library cloned into a weak promoter as the guide RNA target sequence and a mutagenesis plasmid were grown to log phase, and used as the source of host cells to create a flow of host cells through a lagoon. The cells in the lagoon were infected with the diversified selection phage from the overnight incubation. The host cells in the lagoon were contacted with arabinose in order to maintain a high level of expression of the mutagenic genes from the mutagenesis plasmid.

The initial phage titers were about 10⁸ pfu/mL. A PACE experiment was performed for each of the four NNN-PAM libraries ([Doench 1]-[NNN-PAM], [Doench 2]-[NNN-PAM], [G7]-[NNN-PAM], and [VEGF target]-[NNN-PAM] cloned into an accessory plasmid driving pIII expression from a weak promoter as described above. The phage titers were monitored during the PACE experiments. A slow drop in phage titers to 10⁴ pfu/mL was observed. The phage population was isolated from the lagoon at that point, and grown on 2208 host cells containing a separate source of pIII (psp-driven pIII). After this low-stringency propagation period, a 1:100 dilution of the supernatant was added to fresh host cells harboring the accessory plasmid as the only source of pIII in a new lagoon, and the PACE experiments were continued. No drop in phage titers was observed after this low-stringency incubation in 2208 cells.

One exemplary PACE experiment that was run for 72 hours. After that period of time, 24 surviving clones were isolated from the lagoon, sequenced, and characterized. The mutations identified included A262T, K294R, S409I, M694I, E480K, E543D, and E1219V (amino acid numbering according to SEQ ID NO: 9). In another exemplary experiment, surviving clones were isolated after 15 days of incubation. Activity of the identified dVas9 mutants was characterized in a ω-dCas9 luciferase assay. Clones with the best ω-dCas9 fusion protein activity on noncanonical PAM target sequences had the following mutations: E480K, E543D, E1219V, and T1329.

Cas9 Mammalian GFP Activation. Both wild-type dCas9 (SEQ ID NO: 9) and the evolved Cas9 clones were tested in a dCas9-GFP assay in Hek293T cells. The cells were contacted with a reporter construct in which a GFP-encoding sequence was driven from a weak promoter that includes a [gRNA target sequence]-[PAM] sequence. Fusion proteins of dCas9 (wild-type and PACE variants) attached to the transcriptional activator VP64-p65-Rta (VPR) were generated, and the various dCas9-VPR variants were tested for their capacity to activate the GFP reporter in HEK293 cells.

Hek293T were transfected with four separate plasmids: a dCas9-VPR expression plasmid; a plasmid expressing the sgRNA targeting the gRNA target sequence of the GFP reporter plasmid; the GFP reporter plasmid; and an iRFP transfection control. In one experiment, the HEK293 cells were contacted with a GFP reporter included a TAA PAM, and in another experiment, the HEK293 cells were contacted with a population of reporter plasmids containing an NNN PAM library. Cells were harvested 48 hours after transfection and GFP expressing cells were quantified using a BD LSR-FORTESSA cell analyzer.

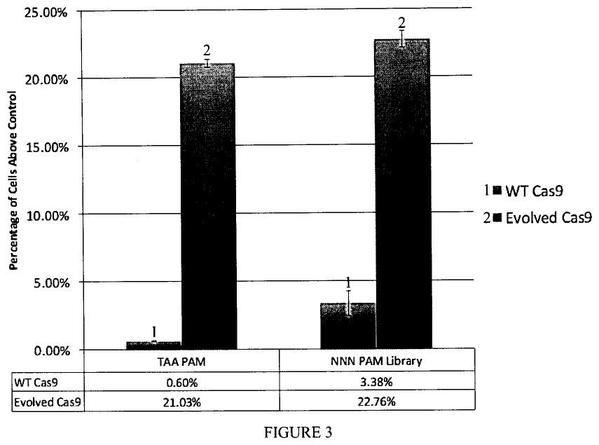

FIG. 3—Cas9 Mammalian GFP Activation. Compared with WT Cas9, the evolved Cas9s showed much higher activity on both the TAA PAM (21.08% vs. 0.60% of cells above negative control) and a library of NNN PAM (22.76% vs. 3.38% of cells above negative control).

Evolved Cas9 Cutting Activity on Target Sequences with Noncanonical PAM.

In order to demonstrate that the PACE mutations universally confer Cas9 activity without PAM restriction, nuclease-active Cas9 proteins were generated based on the sequence provided in, i.e., without the D10A and H840A mutations, but harboring the various PACE mutations. The evolved Cas9 variants were tested in a Cas9 GFP assay, assessing the capacity of the evolved Cas9 protein variants to target and inactivate an emGFP gene integrated into the genome of HEK293 cells using a guide RNA targeting a sequence with a noncanonical PAM. It was observed that 6.45% of cells showed loss of GFP expression when contacted with a wild-type nuclease-active Cas9 (SEQ ID NO: 9), while 54.55% of cells contacted with the evolved Cas9 (E480K, E543D, E1219V, and T1329) showed a loss of GFP expression.

### Example 3: Cas9 Variants without PAM Restrictions

The beneficial mutations conferring Cas9 activity on noncanonical PAM sequences were mapped to a S. pyogenes wild-type sequence. Below is an exemplary Cas9 sequence (S. pyogenes Cas9 with D10 and H840 residues marked with an asterisk following the respective amino acid residues, SEQ ID NO: 9). The D10 and H840 residues of SEQ ID NO: 9 may be mutated to generate a nuclease inactive Cas9 (e.g., to D10A and H840A) or to generate a nickase Cas9 (e.g., to D10A with H840; or to D10 with H840A). The HNH domain (bold and underlined) and the RuvC domain (boxed) are identified. The residues found mutated in the clones isolated from the various PACE experiments, amino acid residues 122, 137, 182, 262, 294, 409, 480, 543, 660, 694, 1219, and 1329 are identified with an asterisk following the respective amino acid residue.

(SEQ ID NO: 9)
MDKKYSIGLD*IGTNSVGWAVITDEYKVPSKKFKVLGNTDRHSIKKNLIGALLFDSGETAEATRLKRTARRRYTRRK
NRICYLQEIFSNEMAKVDDSFFHRLEESELVEEDKKHERHPIFGNI*VDEVAYHEKYPTIYH*LRKKLVDSTDKADL
RLIYLALAHMIKFRGHFLIEGDLNPDNSDVD*KLFIQLVQTYNQLFEENPINASGVDAKAILSARLSKSRRLENLIA
QLPGEKKNGLFGNLIALSLGLTPNFKSNFDLAEDA*KLQLSKDTYDDDLDNLLAQIGDQYADLFLAAK*NLSDAILL
SDILRVNTEITKAPLSASMIKRYDEHHQDLTLLKALVRQQLPEKYKEIFFDQSKNGYAGYIDGGASQEEFYKFIKPI
LEKMDGTEELLVKLNREDLLRKORTFDNGS*IPHQIHLGELHAILRRQEDFYPFLKDNREKIEKILTFRIPYYVGPL
ARGNSRFAWMTRKSEETITPWNFEE*VVDKGASAQSFIERMTNFDKNLPNEKVLPKHSLLYEYFTVYNELTKVKYVT
EGMRKPAFLSGE*QKKAIVDLLFKTNRKVTVKOLKEDYFKKIECFDSVEISGVEDRENASLGTYHDLLKIIKDKDEL
DNEENEDILEDIVLTLTLFEDREMIEERLKTYAHLFDDKVMKQLKRRRYTGWG*RLSRKLINGIRDKQSGKTILDEL

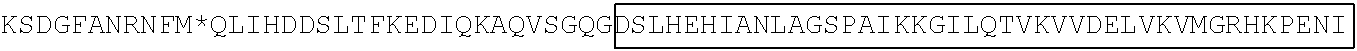

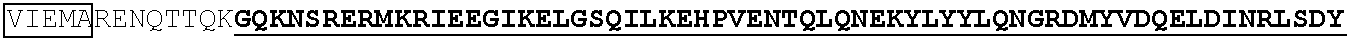

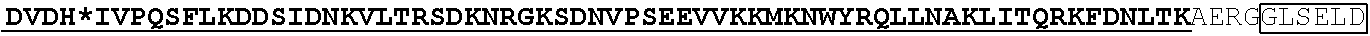

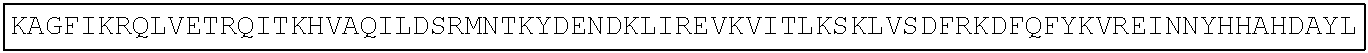

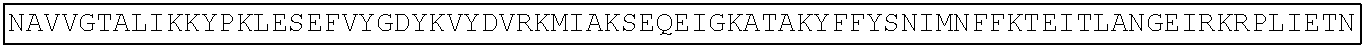

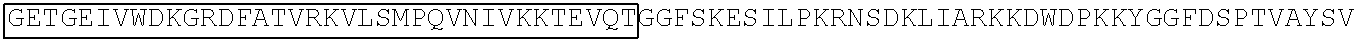

LVVAKVEKGKSKKLKSVKELLGITIMERSSFEKNPIDFLEAKGYKEVKKDLIIKLPKYSLFELENGRKRMLASAGE*
LQKGNELALPSKYVNFLYLASHYEKLKGSPEDNEQKQLFVEQHKHYLDEIIEQISEFSKRVILADANLDKVLSAYNK
HRDKPIREQAENIIHLFTLTNLGAPAAFKYFDT*TIDRKRYTSTKEVLDATLIHQSITGLYETRIDLSQLGGD

The beneficial mutations conferring Cas9 activity on noncanonical PAM sequences were mapped to additional exemplary wild-type Cas9 sequences. The HNH domain (bold and underlined) and the RuvC domain (boxed) are identified. The residues homologous to the residues found mutated in SEQ ID NO: 9, amino acid residues 122, 137, 182, 262, 294, 409, 480, 543, 660, 694, 1219, and 1329 are identified with an asterisk following the respective amino acid residue. In addition, amino acid residues 10 and 840, which are mutated in dCas9 protein variants, are also identified by an asterisk.

(SEQ ID NO: 297)
MDKKYSIGLD*IGTNSVGWAVITDDYKVPSKKFKVLGNTDRHSIKKNLIGALLFGSGETAEATRLKRTARRRYTRRK
NRICYLQEIFSNEMAKVDDSFFHRLEESFLVEEDKKHERHPIFGNI*VDEVAYHEKYPTIYH*LRKKLADSTDKADL
RLIYLALAHMIKFRGHFLIEGDLNPDNSDVD*KLFIQLVQIYNQLFEENPINASRVDAKAILSARLSKSRRLENLIA
QLPGEKRNGLFGNLIALSLGLTPNFKSNFDLAEDA*KLQLSKDTYDDDLDNLLAQIGDQYADLFLAAK*NLSDAILL
SDILRVNSEITKAPLSASMIKRYDEHHQDLTLLKALVRQQLPEKYKEIFFDQSKNGYAGYIDGGASQEEFYKFIKPI
LEKMDGTEELLVKLNREDLLRKORTFDNGS*IPHQIHLGELHAILRRQEDFYPFLKDNREKIEKILTFRIPYYVGPL
ARGNSRFAWMTRKSEETITPWNFEE*VVDKGASAQSFIERMTNFDKNLPNEKVLPKHSLLYEYFTVYNELTKVKYVT
EGMRKPAFLSGE*QKKAIVDLLFKTNRKVTVKOLKEDYFKKIECFDSVEISGVEDRFNASLGAYHDLLKIIKDKDEL
DNEENEDILEDIVLTLTLFEDRGMIEERLKTYAHLFDDKVMKQLKRRRYTGWG*RLSRKLINGIRDKQSGKTILDEL

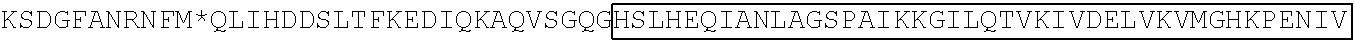

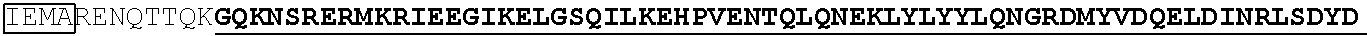

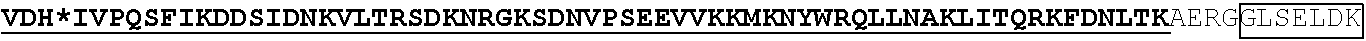

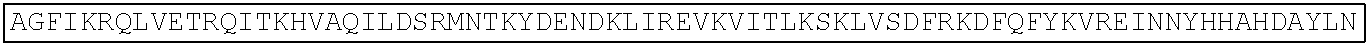

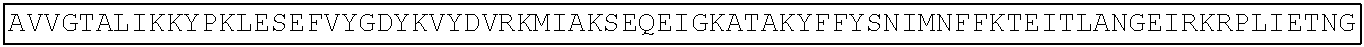

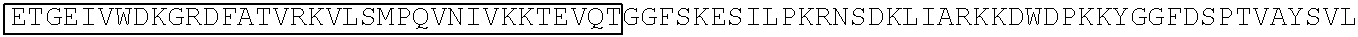

VVAKVEKGKSKKLKSVKELLGITIMERSSFEKNPIDFLEAKGYKEVKKDLIIKLPKYSLFELENGRKRMLASAGE*L
QKGNELALPSKYVNFLYLASHYEKLKGSPEDNEQKQLFVEQHKHYLDEIIEQISEFSKRVILADANLDKVLSAYNKH
RDKPIREQAENIIHLFTLTNLGAPAAFKYFDT* IDRKRYTSTKEVLDATLIHQSITGLYETRIDLSQLGGD
(SEQ ID NO: 298)
MDKKYSIGLA*IGTNSVGWAVITDEYKVPSKKFKVLGNTDRHSIKKNLIGALLFDSGETAEATRLKRTARRRYTRRK
NRICYLQEIFSNEMAKVDDSFFHRLEESFLVEEDKKHERHPIFGNI*VDEVAYHEKYPTIYH*LRKKLVDSTDKADL
RLIYLALAHMIKFRGHFLIEGDLNPDNSDVD*KLFIQLVQTYNQLFEENPINASGVDAKAILSARLSKSRRLENLIA
QLPGEKKNGLFGNLIALSLGLTPNFKSNFDLAEDA*KLQLSKDTYDDDLDNLLAQIGDQYADLFLAAK*NLSDAILL
SDILRVNTEITKAPLSASMIKRYDEHHQDLTLLKALVRQQLPEKYKEIFFDQSKNGYAGYIDGGASQEEFYKFIKPI
LEKMDGTEELLVKLNREDLLRKORTFDNGS*IPHQIHLGELHAILRRQEDFYPFLKDNREKIEKILTFRIPYYVGPL
ARGNSRFAWMTRKSEETITPWNFEE*VVDKGASAQSFIERMTNFDKNLPNEKVLPKHSLLYEYFTVYNELTKVKYVT
EGMRKPAFLSGE*QKKAIVDLLFKTNRKVTVKQLKEDYFKKIECFDSVEISGVEDRENASLGTYHDLLKIIKDKDEL
DNEENEDILEDIVLTLTLFEDREMIEERLKTYAHLFDDKVMKOLKRRRYTGWG*RLSRKLINGIRDKQSGKTILDEL

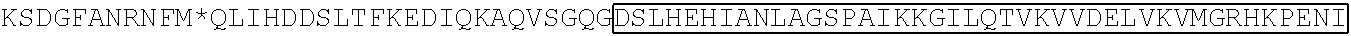

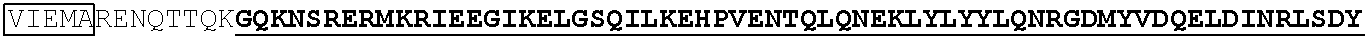

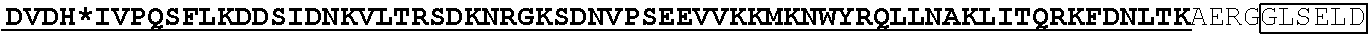

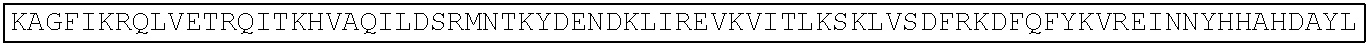

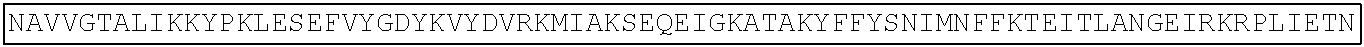

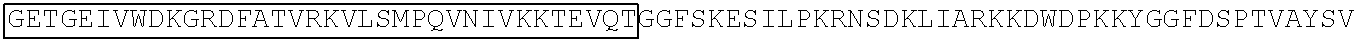

LVVAKVEKGKSKKLKSVKELLGITIMERSSFEKNPIDFLEAKGYKEVKKDLIIKLPKYSLFELENGRKRMLASAGE*
LQKGNELALPSKYVNFLYLASHYEKLKGSPEDNEQKQLFVEQHKHYLDEIIEQISEFSKRVILADANLDKVLSAYNK
HRDKPIREQAENIIHLFTLTNLGAPAAFKYFDT*TIDRKRYTSTKEVLDATLIHQSITGLYETRIDLSQLGGD
(SEQ ID NO: 260)
>gi|504540549|ref WP_014727651.1|type II CRISPR RNA-guided endonuclease Cas9
[Streptococcus thermophilus]
MTKPYSIGLD*IGTNSVGWAVITDNYKVPSKKMKVLGNTSKKYIKKNLLGVLLFDSGITAEGRRLKRTARRRYTRRR
NRILYLQEIFSTEMATLDDAFFQRLDDSFLVPDDKRDSKYPIFGNL*VEEKAYHDEFPTIYH*LRKYLADSTKKADL
RLVYLALAHMIKYRGHFLIEGEFNSKNNDIQ*KNFQDFLDTYNAIFESDLSLENSKQLEEIVKDKISKLEKKDRILK
LFPGEKNSGIFSEFLKLIVGNQADFRKCFNLDEKA*SLHFSKESYDEDLETLLGYIGDDYSDVFLKAK*KLYDAILL
SGFLTVTDNETEAPLSSAMIKRYNEHKEDLALLKEYIRNISLKTYNEVFKDDTKNGYAGYIDGKTNQEDFYVYLKNL
LAEFEGADYFLEKIDREDFLRKORTFDNGS*IPYQIHLQEMRAILDKQAKFYPFLAKNKERIEKILTFRIPYYVGPL
ARGNSDFAWSIRKRNEKITPWNFED*VIDKESSAEAFINRMTSFDLYLPEEKVLPKHSLLYETENVYNELTKVRFIA
ESMRDYQFLDSK*QKKDIVRLYFKDKRKVTDKDIIEYLHAIYGYDGIELKGIEKQFNSSLSTYHDLLNIINDKEFLD
DSSNEAIIEEIIHTLTIFEDREMIKQRLSKFENIFDKSVLKKLSRRHYTGWG*KLSAKLINGIRDEKSGNTILDYLI

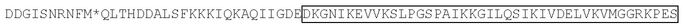

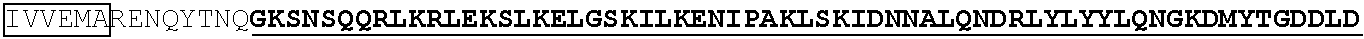

IDRLSNYDIDH*IIPQAFLKDNSIDNKVLVSSASNRGKSDDFPSLEVVKKRKTFWYQLLKSKLISQRFDNTLKAER

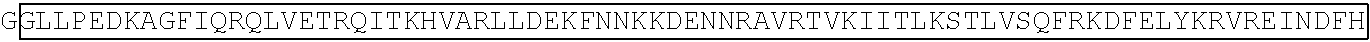

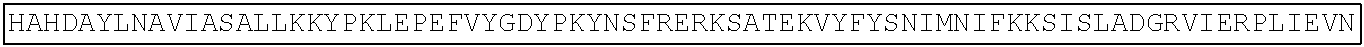

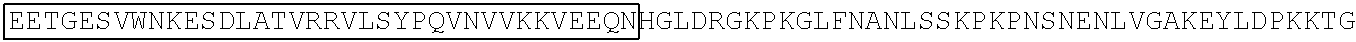

GYAGISNSFAVLVKGTIEKGAKKKITNVLEFQGISILDRINYRKDKLNFLLEKGYKDIELIIELPKYSLFELSDGSR
RMLASILSTNNKRGE*IHKGNQIFLSQKFVKLLYHAKRISNTINENHRKYVENHKKEFEELFYYILEFNENYVGAKK
NGKLLNSAFQSWQNHSIDELCSSFIGPTGSERKGLFELTSRGSAADFEFLGV*KIPRYRDYTPSSLLKDATLIHQSV
TGLYETRIDLAKLGEG|Staphylococcus Aureus Cas9
(SEQ ID NO: 261)
GSHMKRNYILGLD*IGITSVGYGIIDYETRDVIDAGVRLFKEANVENNEGRRSKRGARRLKRRRRHRIQRVKKLLFD
YNLLTDHSELSGINP*YEARVKGLSQKLSEEEFSAALLHLAKRRGVHNVNEVEEDTGNELST*KEQISRNSKALEEK
YVAELQLERLKKDGEVRGSINRFKTSDYVKEAKQLLKVQKAYHQLDQSFIDTYIDLLETRRTYYEGPGEGSPFGWKD
IKEWYEMLMGHCTYFPEELRSVKYAYNADLYNALNDLNNLVITRDENEKLEYY*EKFQIIENVFKQKKKPTLKQIAK
EILVNEEDIKGYRVTSTGKPEFTNLKVYHDIKDITARKEIIENAELLDQIAKILTIYQSSEDIQEELTNLNSELTQE

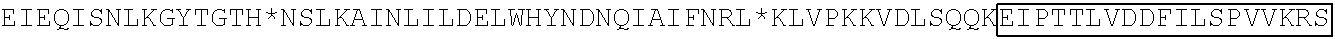

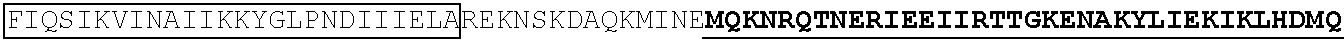

EGKCLYSLEAIPLEDLLNNPFNYEVDH*IIPRSVSFDNSFNNKVLVKQEEASKKGNRTPFQYLSSSDSKISYETFKK

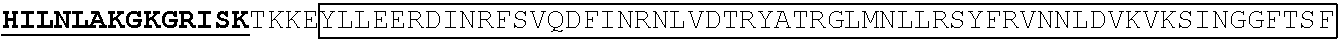

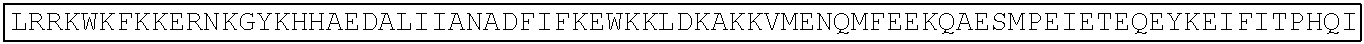

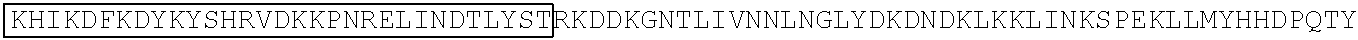

QKLKLIMEQYGDEKNPLYKYYEETGNYLTKYSKKDNGPVIKKIKYYGNKLNAHLDITDDYPNS*RNKVVKLSLKPYR
FDVYLDNGVYKFVTVKNLDVIKKENYYEVNSKAYEEAKKLKKISNQAEFIASFYNNDLIKINGELYRVIGVNNDLLN
RIEVNMIDITYREYLEN*MNDKRPPRIIKTIASKTQSIKKYSTDILGNLYEVKSKKHPQIIKKG

This disclosure provides Cas9 variants in which one or more of the amino acid residues identified by an asterisk are mutated as described herein. In some embodiments, the D10 and H840 residues are mutated, e.g., to an alanine residue, and the Cas9 variants provided include one or more additional mutations of the amino acid residues identified by an asterisk as provided herein. In some embodiments, the D10 residue is mutated, e.g., to an alanine residue, and the Cas9 variants provided include one or more additional mutations of the amino acid residues identified by an asterisk as provided herein.

A number of Cas9 sequences from various species were aligned to determine whether corresponding homologous amino acid residues can be identified in other Cas9 proteins, allowing the generation of Cas9 variants with corresponding mutations of the homologous amino acid residues. The alignment was carried out using the NCBI Constraint-based Multiple Alignment Tool (COBALT (accessible at st-va.ncbi.nlm.nih.gov/tools/cobalt), with the following parameters. Alignment parameters: Gap penalties-11,-1; End-Gap penalties-5,-1. CDD Parameters: Use RPS BLAST on; Blast E-value 0.003; Find Conserved columns and Recompute on. Query Clustering Parameters: Use query clusters on; Word Size 4; Max cluster distance 0.8; Alphabet Regular.

An exemplary alignment of four Cas9 sequences is provided below. The Cas9 sequences in the alignment are: Sequence 1 (S1): SEQ ID NO: 10|WP_010922251| gi 499224711|type II CRISPR RNA-guided endonuclease Cas9 [Streptococcus pyogenes]; Sequence 2 (S2): SEQ ID NO: 11|WP_039695303|gi 746743737|type II CRISPR RNA-guided endonuclease Cas9 [Streptococcus gallolyticus]; Sequence 3 (S3): SEQ ID NO: 12| WP_045635197|gi 782887988|type II CRISPR RNA-guided endonuclease Cas9 [Streptococcus mitis]; Sequence 4 (S4): SEQ ID NO: 13|5AXW_A|gi 924443546|Staphylococcus Aureus Cas9. The HNH domain (bold and underlined) and the RuvC domain (boxed) are identified for each of the four sequences. Amino acid residues 10, 122, 137, 182, 262, 294, 409, 480, 543, 660, 694, 840, 1219, and 1329 in S1 and the homologous amino acids in the aligned sequences are identified with an asterisk following the respective amino acid residue.

S1 1 --MDKK-YSIGLD*IGTNSVGWAVITDEYKVPSKKFKVLGNTDRHSIKKNLI--GALLFDSG--ETAEATRLKRTARRRYT 73
S2 1 --MTKKNYSIGLD*IGTNSVGWAVITDDYKVPAKKMKVLGNTDKKYIKKNLL--GALLFDSG--ETAEATRLKRTARRRYT 74
S3 1 --M-KKGYSIGLD*IGTNSVGFAVITDDYKVPSKKMKVLGNTDKRFIKKNLI--GALLFDEG--TTAEARRLKRTARRRYT 73
S4 1 GSHMKRNYILGLD*IGITSVGYGII--DYET-----------------RDVIDAGVRLFKEANVENNEGRRSKRGARRLKR 61
S1 74 RRKNRICYLQEIFSNEMAKVDDSFFHRLEESFLVEEDKKHERHPIFGNI*VDEVAYHEKYPTIYH*LRKKLVDSTDKADLRL 153
S2 75 RRKNRLRYLQEIFANEIAKVDESFFQRLDESFLTDDDKTFDSHPIFGNK*AEEDAYHQKFPTIYH*LRKHLADSSEKADIRL 154
S3 74 RRKNRLRYLQEIFSEEMSKVDSSFFHRLDDSFLIPEDKRESKYPIFATL*TEEKEYHKQFPTIYH*LRKQLADSKEKTDLRL 153
S4 62 RRRHRIQRVKKLL--------------------------FDYNLLTD-------- HSELSGINP*YEARVKGLSQKLSEEE 107
S1 154 IYLALAHMIKFRGHFLIEGDLNPDNSDVD*KLFIQLVQTYNQLFEENPINASGVDAKAILSARLSKSRRLENLIAQLPGEK 233
S2 155 VYLALAHMIKFRGHFLIEGELNAENTDVQ*KIFADFVGVYNRTFDDSHLSEITVDVASILTEKISKSRRLENLIKYYPTEK 234
S3 154 IYLALAHMIKFRGHFLIEGDLNPDNSDVD*KLFIQLVQTYNQLFEENPINASGVDAKAILSARLSKSRRLENLIAQLPGEK 233
S4 108 FSAALLHLAKRRG------------------------------------------VHNVNEVEEDT----------------131
S1 234 KNGLFGNLIALSLGLTPNFKSNFDLAEDA*KLQISKDTYDDDLDNLLAQIGDQYADLFLAAK*NLSDAILLSDILRVNTEIT 313
S2 235 KNTLFGNLIALALGLQPNFKTNFKLSEDA*KLQFSKDTYEEDLEELLGKIGDDYADLFTSAK*NLYDAILLSGILTVDDNST 314
S3 234 STGLFSEFLKLIVGNQADFKKHFDLEDKA*PLQFSKDTYDEDLENLLGQIGDDFTDLFVSAK*KLYDAILLSGILTVTDPST 313
S4 132 ---------GNELS-----------------------------------T*KEQISRN------------------------ 144
S1 314 KAPLSASMIKRYDEHHQDLTLLKALVRQQLPEKYKEIFFDQSKNGYAGYIDGGASQEEFYKFIKPILERM---DGTEELLV 391
S2 315 KAPLSASMIKRYVEHHEDLEKLKEFIKANKSELYHDIFKDKNKNGYAGYIENGVKQDEFYKYLKNILLSKIKIDGSDYFLD 394
S3 314 KAPLSASMIERYENHQNDLAALKQFIKNNLPEKYDEVFSDQSKDGYAGYIDGTTQETFYKYIKNLLISKF---EGTDYFLD 391
S4 145 -------SKALEEKYVAELQ---------------------------------------- -------LERLKKDG----- 165
S1 392 KLNREDLLRKQRTFDNGS*IPHQIHLGELHAILRRQEDFYPFLKDNREKIEKILTFRIPYYVGPLARGNSRFAWMTRKSEE 471
S2 395 KIEREDFLRKQRTFDNGS*IPHQIHLQEMHAILRRQGDYYPFLKEKQDRIEKILTFRIPYYVGPLVRKDSRFAWAEYRSDE 474
S3 392 KIEREDFLRKQRTFDNGS*IPHQIHLQEMNAILRRQEYYPFLYKDNKEKIEKILTFRIPYYVGPLARGNRDFAWLTRNSDE 471
S4 166 --EVRGSINRFKTSD-------------YVKEAKQLLKVQKAYHQLQSFIDTYIDLLETRRTYYEGP--GEGSPFGW---K 227
S1 472 TITPWNFEE*VVDKGASAQSFIERMTNFDKNLPNEKVLPKHSLLYEYFTVYNELTKVKYVTEGMRKPAFLSGE*QKKAIVDL 551
S2 475 KITPWNFDK*VIDKEKSAEKFITRMTLNDLYLPEEKVLPKHSHVYETYAVYNELTKIKYVNEQGKE-SFFDSN*MKQEIFDH 553
S3 472 AIRPWNFEE*IVDKASSAEDFINKMTNYDLYLPEEKVLPKHSLLYETFAVYNELTKVKFIAEGLRDYQFLDSG*QKKQIVNQ 551
S4 228 DIKEW-----------------YEMLMGHCTYFPEELRSVKYAYNADLYNALNDLNNLVITRDENEK--LEYY*EKFQIIEN 289
S1 552 LFKTNRKVTVKQLKEDYFKKIECFDSVEISGVEDR---FNASLGTYHDLLKIIKDKDFLDNEENEDILEDIVLTLTLFED 628
S2 554 VFKENRKVTKEKLLNYLNKEPPEYRIKDLIGLDKENKSFNASLGTYHDLKKIL-DKAFLDDKVNEEVIEDIIKTLTLFED 632
S3 552 LFKENRKVTEKDIIHYLHN-VDGYDGIELKGIEKQ---FNASLSTYHDLLKIIKDKEFMDDAKNEAILENIVHTLTIFED 627
S4 290 VFKQKKKPTLKQIAKEILVNEEDIKGYRVTSTGKPEF---TNLKVYHDIKDITARKEII---ENAELLDQIAKILTIYQS 363
S1 629 REMIEERLKTYAHLFDDKVMKQLKR-RRYTGWG*RLSRRLINGIRDKQSGKTILDFLKSDGFANRNFM*QLIHDDSLTFRED 707
S2 633 KDMIHERLQKYSDIFTANQLKKLER-RHYTGWG*RLSYKLINGIRNKENNKTILDYLIDDGSANRNFM*QLINDDTLPFKQI 711
S3 628 REMIKQRLAQYDSLFDERVIKALTR-RHYTGWG*KLSAKLINGICDKQTGNTILDYLIDDGKINRNFM*QLINDDGLSFKEI 706
S4 364 SEDIQEELTNLNSELTQEEIEQISNLKGYGTH*NLSLKAINLILDE--------LWTNDNQIAIFNRL*KLVP--------- 428

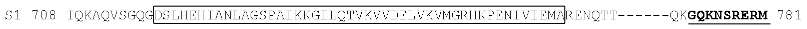

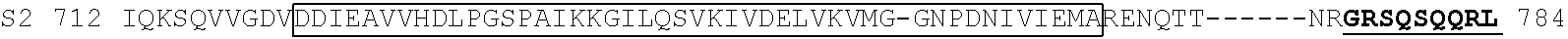

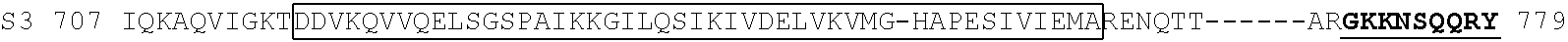

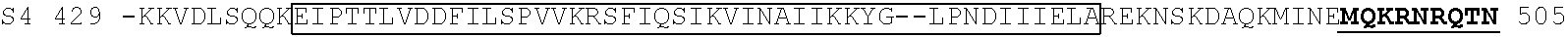

S1 782 KRIEEGIKELGSQIL-----KEIHPVENTQLQNEKLYLYYLQNGRDMYVDQELDINRLSD-----YDVDH*IVPQSFLKDD 850
S2 785 KKLQNSLKELGSNILNEEKPSYIEDKVENSHLQNDQLFLYYIQNGKDMYTGDELDIDHLSD----YDIDH*IIPQAFIKDD 860
S3 780 KRIEDSLKILASGL---DSNILKENPTDNNQLQNDRLFLYYLQNGKDMYTGEALDINGLSS----YDIDH*IIPQAFIKDD 852
S4 506 ERIEKIIRTTGK---------------ENAKYLIEKIKLHDMQEGKCLYSLEAIPLEDLLNNPFNYEVDH*IIPRSVSFDN 570

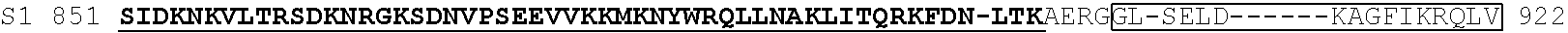

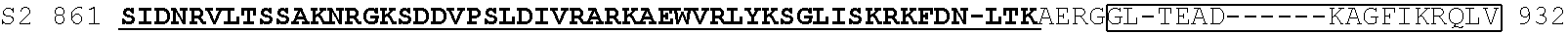

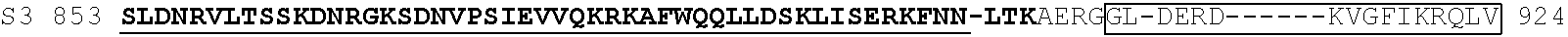

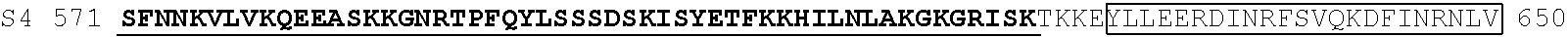

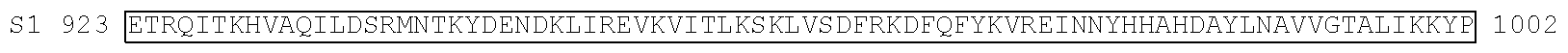

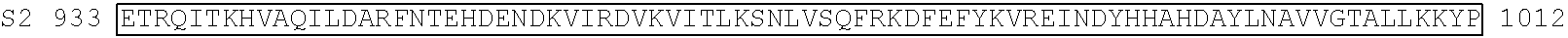

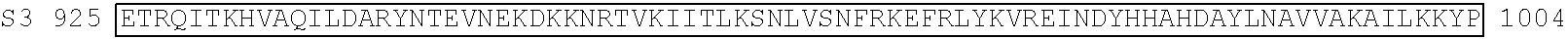

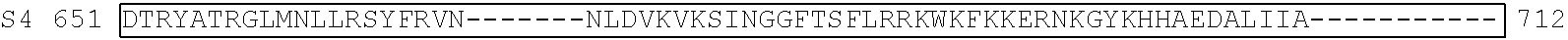

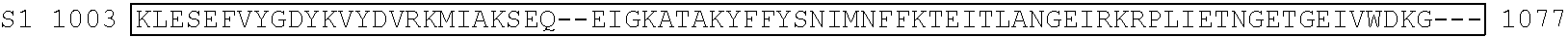

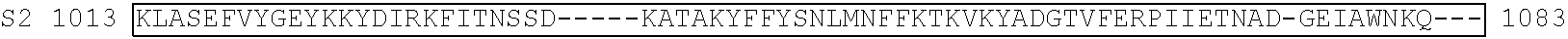

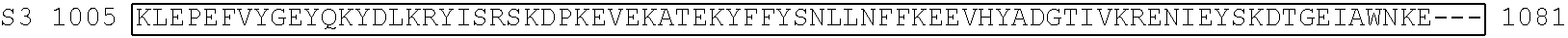

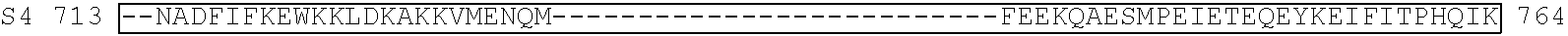

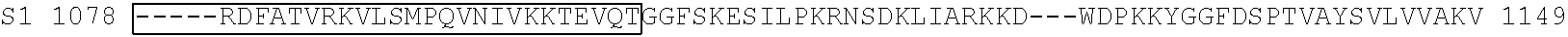

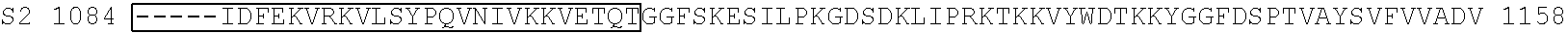

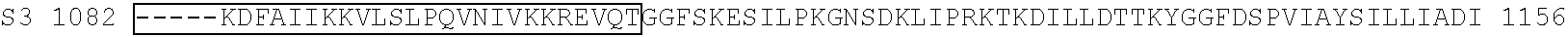

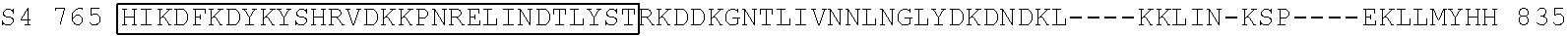

S1 1150 EKGKSKKLKSVKELLGITIMERSSFEKNPI-DFLEAKG----YKEVKKDLIIKLPKYSLFELENGRKRMLASAGE*LQKG 1223
S2 1159 EKGKAKKLKTVKELVGISIMERSFFEENPV-EFLENKG----YHNIREDKLIKLPKYSLFEFEGGRRRLLASASE*LQKG 1232
S3 1157 EKGKAKKLKTVKTLVGITIMEKAAFEENPI-TFLENKG----YHNVRKENILCLPKYSLFELENGRRRLLASAKE*LQKG 1230
S4 836 DPQTYQKLK--------LIMEQYGDEKNPLYKYYEETGNYLTKYSKKDNGPIKKIKYYGNKLNAHLDITDDYPNS*RNKV 907
S1 1224 NELALPSKYVNFLYLASHYEKLKGSPEDNEQKQLFVEQHKHYLDEIIEQISEFSKRVILADANLDKVLSAYNKH------ 1297
S2 1233 NEMVLPGYLVELLYHAHRADNF----NSTEYLNYVSEHKKEFEKVLSCVEDFANLYVDVEKNLSKIRAVADSM------- 1301
S3 1231 NEIVLPVYLTTLLYHSKNVHKL----DEPGHLEYIQKHRNEFKDLLNLVSEFSQKYVLADANLEKIKSLYADN------- 1299
S4 908 VKLSLKPYRFD-VYLDNGVYKFV----TVKNLDVIK--KENYYEVNSKAYEEAKKLKKISNQAEFIASFYNNDLIKING 979
S1 1298 RDKPIREQAENIIHLFTLTNLGAPAAFKYFDT*TIDRKRYTSTKEVLDATLIHQSIT---------GLYETRI---DLSQL 1365
S2 1302 DNFSIEEISNSFINLLTLTALGAPADFNFLGE*KIPRKRYTSTKECINATLIHQSIT--------GLYETRI----DLSKL 1369
S3 1300 EQADIEILANSFINLLTFTALGAPAAFKFFGK*DIDRKRYTTVSEILNATLIHQSIT-------GLYTETWI----DLSKL 1367
S4 980 ELYRVIGVNNDLLNRIEVNMIDITYR-EYLEN*MNDKRPPRIIKTIASKT---QSIKKYSTDILGNLYEVKSKKHPQIIKK 1055
S1 1366 GGD 1368
S2 1370 GEE 1372
S3 1368 GED 1370
S4 1056 G00 1056

The alignment demonstrates that amino acid sequences and amino acid residues that are homologous to a reference Cas9 amino acid sequence or amino acid residue can be identified across Cas9 sequence variants, including, but not limited to Cas9 sequences from different species, by identifying the amino acid sequence or residue that aligns with the reference sequence or the reference residue using alignment programs and algorithms known in the art. This disclosure provides Cas9 variants in which one or more of the amino acid residues identified by an asterisk in SEQ ID NO: 9 are mutated as described herein. The residues in Cas9 sequences other than SEQ ID NO: 9 that correspond to the residues identified in SEQ ID NO: 9 by an asterisk are referred to herein as “homologous” or “corresponding” residues. Such homologous residues can be identified by sequence alignment, e.g., as described above, and by identifying the sequence or residue that aligns with the reference sequence or residue. Similarly, mutations in Cas9 sequences other than SEQ ID NO: 9 that correspond to mutations identified in SEQ ID NO: 9 herein, e.g., mutations of residues 10, 122, 137, 182, 262, 294, 409, 480, 543, 660, 694, 840, 1219, and 1329 in SEQ ID NO: 9, are referred to herein as “homologous” or “corresponding” mutations. For example, the mutations corresponding to the D10A mutation in S1 for the four aligned sequences above are D10A for S2, D9A for S3, and D13A for S4; the corresponding mutations for H840A in S1 are H850A for S2, H842A for S3, and H560 for S4; the corresponding mutation for X1219V in S1 are X1228V for S2, X1226 for S3, and X903V for S4, and so on.

A total of 250 Cas9 sequences (SEQ ID NOs: 10-262) from different species were aligned using the same algorithm and alignment parameters outlined above. Amino acid residues homologous to residues 10, 122, 137, 182, 262, 294, 409, 480, 543, 660, 694, 840, 1219, and 1329 were identified in the same manner as outlined above. The alignments are provided below. The HNH domain (bold and underlined) and the RuvC domain (boxed) are identified for each of the four sequences. Residues corresponding to amino acid residues 10, 122, 137, 182, 262, 294, 409, 480, 543, 660, 694, 840, 1219, and 1329 in SEQ ID NO: 9 are boxed in SEQ ID NO: 10 in the alignments, allowing for the identification of the corresponding amino acid residues in the aligned sequences.

Cas9 variants with one or more mutations in amino acid residues homologous to amino acid residues 122, 137, 182, 262, 294, 409, 480, 543, 660, 694, 1219, and 1329 of SEQ ID NO: 9 are provided herein. In some embodiments, the Cas9 variants provided herein comprise mutations corresponding to the D10A and the H840A mutations in SEQ ID NO: 9, resulting in a nuclease-inactive dCas9, and at least one, at least two, at least three, at least four, at least five, at least six, or at least seven mutations of amino acid residues homologous to amino acid residues 122, 137, 182, 262, 294, 409, 480, 543, 660, 694, 1219, and 1329 of SEQ ID NO: 9.

Cas9 variants with one or more mutations in amino acid residues homologous to amino acid residues 122, 137, 182, 262, 294, 409, 480, 543, 660, 694, 1219, and 1329 of SEQ ID NO: 9 are provided herein. In some embodiments, the Cas9 variants provided herein comprise mutations corresponding to the D10A mutations in SEQ ID NO: 9, resulting in a partially nuclease-inactive dCas9, wherein the Cas9 can nick the non-target strand but not the targeted strand, and at least one, at least two, at least three, at least four, at least five, at least six, or at least seven mutations of amino acid residues homologous to amino acid residues 122, 137, 182, 262, 294, 409, 480, 543, 660, 694, 1219, and 1329 of SEQ ID NO: 9.

| WP_010922251.1 | type II CRISPR RNA-guided endonuclease Cas9 [Streptococcus pyogenes] SEQ ID NO: 10 | | |
|---|---|---|---|
| WP_039695303.1 | type II CRISPR RNA-guided endonuclease Cas9 [Streptococcus gallolyticus] SEQ ID NO: 11 | | |
| WP_045635197.1 | type II CRISPR RNA-guided endonuclease Cas9 [Streptococcus mitis] SEQ ID NO: 12 | | |
| 5AXW_A | Cas9, Chain A, Crystal Structure [Staphylococcus Aureus] SEQ ID NO: 13 | | |
| WP_009880683.1 | type II CRISPR RNA-guided endonuclease Cas9 [Streptococcus pyogenes] SEQ ID NO: 14 | | |
| WP_010922251.1 | type II CRISPR RNA-guided endonuclease Cas9 [Streptococcus pyogenes] SEQ ID NO: 15 | | |
| WP_011054416.1 | type II CRISPR RNA-guided endonuclease Cas9 [Streptococcus pyogenes] SEQ ID NO: 16 | | |
| WP_011284745.1 | type II CRISPR RNA-guided endonuclease Cas9 [Streptococcus pyogenes] SEQ ID NO: 17 | | |
| WP_011285506.1 | type II CRISPR RNA-guided endonuclease Cas9 [Streptococcus pyogenes] SEQ ID NO: 18 | | |
| WP_011527619.1 | type II CRISPR RNA-guided endonuclease Cas9 [Streptococcus pyogenes] SEQ ID NO: 19 | | |
| WP_012560673.1 | type II CRISPR RNA-guided endonuclease Cas9 [Streptococcus pyogenes] SEQ ID NO: 20 | | |
| WP_014407541.1 | type II CRISPR RNA-guided endonuclease Cas9 [Streptococcus pyogenes] SEQ ID NO: 21 | | |
| WP_020905136.1 | type II CRISPR RNA-guided endonuclease Cas9 [Streptococcus pyogenes] SEQ ID NO: 22 | | |
| WP_023080005.1 | type II CRISPR RNA-guided endonuclease Cas9 [Streptococcus pyogenes] SEQ ID NO: 23 | | |
| WP_023610282.1 | type II CRISPR RNA-guided endonuclease Cas9 [Streptococcus pyogenes] SEQ ID NO: 24 | | |
| WP_030125963.1 | type II CRISPR RNA-guided endonuclease Cas9 [Streptococcus pyogenes] SEQ ID NO: 25 | | |
| WP_030126706.1 | type II CRISPR RNA-guided endonuclease Cas9 [Streptococcus pyogenes] SEQ ID NO: 26 | | |
| WP_031488318.1 | type II CRISPR RNA-guided endonuclease Cas9 [Streptococcus pyogenes] SEQ ID NO: 27 | | |
| WP_032460140.1 | type II CRISPR RNA-guided endonuclease Cas9 [Streptococcus pyogenes] SEQ ID NO: 28 | | |
| WP_032461047.1 | type II CRISPR RNA-guided endonuclease Cas9 [Streptococcus pyogenes] SEQ ID NO: 29 | | |
| WP_032462016.1 | type II CRISPR RNA-guided endonuclease Cas9 [Streptococcus pyogenes] SEQ ID NO: 30 | | |
| WP_032462936.1 | type II CRISPR RNA-guided endonuclease Cas9 [Streptococcus pyogenes] SEQ ID NO: 31 | | |
| WP_032464890.1 | type II CRISPR RNA-guided endonuclease Cas9 [Streptococcus pyogenes] SEQ ID NO: 32 | | |
| WP_033888930.1 | type II CRISPR RNA-guided endonuclease Cas9 [Streptococcus pyogenes] SEQ ID NO: 33 | | |
| WP_038431314.1 | type II CRISPR RNA-guided endonuclease Cas9 [Streptococcus pyogenes] SEQ ID NO: 34 | | |
| WP_038432938.1 | type II CRISPR RNA-guided endonuclease Cas9 [Streptococcus pyogenes] SEQ ID NO: 35 | | |
| WP_038434062.1 | type II CRISPR RNA-guided endonuclease Cas9 [Streptococcus pyogenes] SEQ ID NO: 36 | | |
| BAQ51233.1 | CRISPR-associated protein, Csn1 family [Streptococcus pyogenes] SEQ ID NO: 37 | | |
| KGE60162.1 | hypothetical protein MGAS2111_0903 [Streptococcus pyogenes MGAS2111] SEQ ID NO: 38 | | |
| KGE60856.1 | CRISPR-associated endonuclease protein [Streptococcus pyogenes SS1447] SEQ ID NO: 39 | | |
| WP_002989955.1 | MULTISPECIES: type II CRISPR RNA-guided endonuclease Cas9 [Streptococcus] SEQ ID NO: 40 | | |
| WP_003030002.1 | MULTISPECIES: type II CRISPR RNA-guided endonuclease Cas9 [Streptococcus] SEQ ID NO: 41 | | |
| WP_003065552.1 | MULTISPECIES: type II CRISPR RNA-guided endonuclease Cas9 [Streptococcus] SEQ ID NO: 42 | | |
| WP_001040076.1 | type II CRISPR RNA-guided endonuclease Cas9 [Streptococcus agalactiae] SEQ ID NO: 43 | | |
| WP_001040078.1 | type II CRISPR RNA-guided endonuclease Cas9 [Streptococcus agalactiae] SEQ ID NO: 44 | | |
| WP_001040080.1 | type II CRISPR RNA-guided endonuclease Cas9 [Streptococcus agalactiae] SEQ ID NO: 45 | | |
| WP_001040081.1 | type II CRISPR RNA-guided endonuclease Cas9 [Streptococcus agalactiae] SEQ ID NO: 46 | | |
| WP_001040083.1 | type II CRISPR RNA-guided endonuclease Cas9 [Streptococcus agalactiae] SEQ ID NO: 47 | | |
| WP_001040085.1 | type II CRISPR RNA-guided endonuclease Cas9 [Streptococcus agalactiae] SEQ ID NO: 48 | | |
| WP_001040087.1 | type II CRISPR RNA-guided endonuclease Cas9 [Streptococcus agalactiae] SEQ ID NO: 49 | | |
| WP_001040088.1 | type II CRISPR RNA-guided endonuclease Cas9 [Streptococcus agalactiae] SEQ ID NO: 50 | | |
| WP_001040089.1 | type II CRISPR RNA-guided endonuclease Cas9 [Streptococcus agalactiae] SEQ ID NO: 51 | | |
| WP_001040090.1 | type II CRISPR RNA-guided endonuclease Cas9 [Streptococcus agalactiae] SEQ ID NO: 52 | | |
| WP_001040091.1 | type II CRISPR RNA-guided endonuclease Cas9 [Streptococcus agalactiae] SEQ ID NO: 53 | | |
| WP_001040092.1 | type II CRISPR RNA-guided endonuclease Cas9 [Streptococcus agalactiae] SEQ ID NO: 54 | | |
| WP_001040094.1 | type II CRISPR RNA-guided endonuclease Cas9 [Streptococcus agalactiae] SEQ ID NO: 55 | | |
| WP_001040095.1 | type II CRISPR RNA-guided endonuclease Cas9 [Streptococcus agalactiae] SEQ ID NO: 56 | | |
| WP_001040096.1 | type II CRISPR RNA-guided endonuclease Cas9 [Streptococcus agalactiae] SEQ ID NO: 57 | | |
| WP_001040097.1 | type II CRISPR RNA-guided endonuclease Cas9 [Streptococcus agalactiae] SEQ ID NO: 58 | | |
| WP_001040098.1 | type II CRISPR RNA-guided endonuclease Cas9 [Streptococcus agalactiae] SEQ ID NO: 59 | | |
| WP_001040099.1 | type II CRISPR RNA-guided endonuclease Cas9 [Streptococcus agalactiae] SEQ ID NO: 60 | | |
| WP_001040100.1 | type II CRISPR RNA-guided endonuclease Cas9 [Streptococcus agalactiae] SEQ ID NO: 61 | | |
| WP_001040104.1 | type II CRISPR RNA-guided endonuclease Cas9 [Streptococcus agalactiae] SEQ ID NO: 62 | | |
| WP_001040105.1 | type II CRISPR RNA-guided endonuclease Cas9 [Streptococcus agalactiae] SEQ ID NO: 63 | | |
| WP_001040106.1 | type II CRISPR RNA-guided endonuclease Cas9 [Streptococcus agalactiae] SEQ ID NO: 64 | | |
| WP_001040107.1 | type II CRISPR RNA-guided endonuclease Cas9 [Streptococcus agalactiae] SEQ ID NO: 65 | | |
| WP_001040108.1 | type II CRISPR RNA-guided endonuclease Cas9 [Streptococcus agalactiae] SEQ ID NO: 66 | | |
| WP_001040109.1 | type II CRISPR RNA-guided endonuclease Cas9 [Streptococcus agalactiae] SEQ ID NO: 67 | | |
| WP_001040110.1 | type II CRISPR RNA-guided endonuclease Cas9 [Streptococcus agalactiae] SEQ ID NO: 68 | | |
| WP_015058523.1 | type II CRISPR RNA-guided endonuclease Cas9 [Streptococcus agalactiae] SEQ ID NO: 69 | | |
| WP_017643650.1 | type II CRISPR RNA-guided endonuclease Cas9 [Streptococcus agalactiae] SEQ ID NO: 70 | | |
| WP_017647151.1 | type II CRISPR RNA-guided endonuclease Cas9 [Streptococcus agalactiae] SEQ ID NO: 71 | | |
| WP_017648376.1 | type II CRISPR RNA-guided endonuclease Cas9 [Streptococcus agalactiae] SEQ ID NO: 72 | | |
| WP_017649527.1 | type II CRISPR RNA-guided endonuclease Cas9 [Streptococcus agalactiae] SEQ ID NO: 73 | | |
| WP_017771611.1 | type II CRISPR RNA-guided endonuclease Cas9 [Streptococcus agalactiae] SEQ ID NO: 74 | | |
| WP_017771984.1 | type II CRISPR RNA-guided endonuclease Cas9 [Streptococcus agalactiae] SEQ ID NO: 75 | | |
| CFQ25032.1 | CRISPR-associated protein [Streptococcus agalactiae] SEQ ID NO: 76 | | |
| CFV16040.1 | CRISPR-associated protein [Streptococcus agalactiae] SEQ ID NO: 77 | | |
| KLJ37842.1 | CRISPR-associated protein Csn1 [Streptococcus agalactiae] SEQ ID NO: 78 | | |
| KLJ72361.1 | CRISPR-associated protein Csn1 [Streptococcus agalactiae] SEQ ID NO: 79 | | |
| KLL20707.1 | CRISPR-associated protein Csn1 [Streptococcus agalactiae] SEQ ID NO: 80 | | |
| KLL42645.1 | CRISPR-associated protein Csn1 [Streptococcus agalactiae] SEQ ID NO: 81 | | |
| WP_047207273.1 | type II CRISPR RNA-guided endonuclease Cas9 [Streptococcus agalactiae] SEQ ID NO: 82 | | |
| WP_047209694.1 | type II CRISPR RNA-guided endonuclease Cas9 [Streptococcus agalactiae] SEQ ID NO: 83 | | |
| WP_050198062.1 | type II CRISPR RNA-guided endonuclease Cas9 [Streptococcus agalactiae] SEQ ID NO: 84 | | |
| WP_050201642.1 | type II CRISPR RNA-guided endonuclease Cas9 [Streptococcus agalactiae] SEQ ID NO: 85 | | |
| WP_050204027.1 | type II CRISPR RNA-guided endonuclease Cas9 [Streptococcus agalactiae] SEQ ID NO: 86 | | |
| WP_050881965.1 | type II CRISPR RNA-guided endonuclease Cas9 [Streptococcus agalactiae] SEQ ID NO: 87 | | |
| WP_050886065.1 | type II CRISPR RNA-guided endonuclease Cas9 [Streptococcus agalactiae] SEQ ID NO: 88 | | |
| AHN30376.1 | CRISPR-associated protein Csn1 [Streptococcus agalactiae 138P] SEQ ID NO: 89 | | |
| EAO78426.1 | reticulocyte binding protein [Streptococcus agalactiae H36B] SEQ ID NO: 90 | | |
| CCW42055.1 | CRISPR-associated protein, SAG0894 family [Streptococcus agalactiae ILRI112] SEQ ID NO: 91 | | |
| WP_003041502.1 | type II CRISPR RNA-guided endonuclease Cas9 [Streptococcus anginosus] SEQ ID NO: 92 | | |
| WP_037593752.1 | type II CRISPR RNA-guided endonuclease Cas9 [Streptococcus anginosus] SEQ ID NO: 93 | | |
| WP_049516684.1 | CRISPR-associated protein Csn1 [Streptococcus anginosus] SEQ ID NO: 94 | | |
| GAD46167.1 | hypothetical protein ANG6_0662 [Streptococcus anginosus T5] SEQ ID NO: 95 | | |
| WP_018363470.1 | type II CRISPR RNA-guided endonuclease Cas9 [Streptococcus caballi] SEQ ID NO: 96 | | |
| WP_003043819.1 | type II CRISPR RNA-guided endonuclease Cas9 [Streptococcus canis] SEQ ID NO: 97 | | |
| WP_006269658.1 | type II CRISPR RNA-guided endonuclease Cas9 [Streptococcus constellatus] SEQ ID NO: 98 | | |
| WP_048800889.1 | type II CRISPR RNA-guided endonuclease Cas9 [Streptococcus constellatus] SEQ ID NO: 99 | | |
| WP_012767106.1 | type II CRISPR RNA-guided endonuclease Cas9 [Streptococcus dysgalactiae] SEQ ID NO: 100 | | |
| WP_014612333.1 | type II CRISPR RNA-guided endonuclease Cas9 [Streptococcus dysgalactiae] SEQ ID NO: 101 | | |
| WP_015017095.1 | type II CRISPR RNA-guided endonuclease Cas9 [Streptococcus dysgalactiae] SEQ ID NO: 102 | | |
| WP_015057649.1 | type II CRISPR RNA-guided endonuclease Cas9 [Streptococcus dysgalactiae] SEQ ID NO: 103 | | |
| WP_048327215.1 | type II CRISPR RNA-guided endonuclease Cas9 [Streptococcus dysgalactiae] SEQ ID NO: 104 | | |
| WP_049519324.1 | CRISPR-associated protein Csn1 [Streptococcus dysgalactiae] SEQ ID NO: 105 | | |
| WP_012515931.1 | type II CRISPR RNA-guided endonuclease Cas9 [Streptococcus equi] SEQ ID NO: 106 | | |
| WP_021320964.1 | type II CRISPR RNA-guided endonuclease Cas9 [Streptococcus equi] SEQ ID NO: 107 | | |
| WP_037581760.1 | type II CRISPR RNA-guided endonuclease Cas9 [Streptococcus equi] SEQ ID NO: 108 | | |
| WP_004232481.1 | type II CRISPR RNA-guided endonuclease Cas9 [Streptococcus equinus] SEQ ID NO: 109 | | |
| WP_009854540.1 | type II CRISPR RNA-guided endonuclease Cas9 [Streptococcus gallolyticus] SEQ ID NO: 110 | | |
| WP_012962174.1 | type II CRISPR RNA-guided endonuclease Cas9 [Streptococcus gallolyticus] SEQ ID NO: 111 | | |
| WP_039695303.1 | type II CRISPR RNA-guided endonuclease Cas9 [Streptococcus gallolyticus] SEQ ID NO: 112 | | |
| WP_014334983.1 | type II CRISPR RNA-guided endonuclease Cas9 [Streptococcus infantarius] SEQ ID NO: 113 | | |
| WP_003099269.1 | type II CRISPR RNA-guided endonuclease Cas9 [Streptococcus iniae] SEQ ID NO: 114 | | |
| AHY15608.1 | CRISPR-associated protein Csn1 [Streptococcus iniae] SEQ ID NO: 115 | | |
| AHY17476.1 | CRISPR-associated protein Csn1 [Streptococcus iniae] SEQ ID NO: 116 | | |
| ESR09100.1 | hypothetical protein IUSA1_08595 [Streptococcus iniae IUSA1] SEQ ID NO: 117 | | |
| AGM98575.1 | CRISPR-associated protein Cas9/Csnl, subtype II/NMEMI [Streptococcus iniae SF1] SEQ ID NO: 118 | | |
| ALF27331.1 | CRISPR-associated protein Csn1 [Streptococcus intermedius] SEQ ID NO: 119 | | |
| WP_018372492.1 | type II CRISPR RNA-guided endonuclease Cas9 [Streptococcus massiliensis] SEQ ID NO: 120 | | |
| WP_045618028.1 | type II CRISPR RNA-guided endonuclease Cas9 [Streptococcus mitis] SEQ ID NO: 121 | | |
| WP_045635197.1 | type II CRISPR RNA-guided endonuclease Cas9 [Streptococcus mitis] SEQ ID NO: 122 | | |
| WP_002263549.1 | type II CRISPR RNA-guided endonuclease Cas9 [Streptococcus mutans] SEQ ID NO: 123 | | |
| WP_002263887.1 | type II CRISPR RNA-guided endonuclease Cas9 [Streptococcus mutans] SEQ ID NO: 124 | | |
| WP_002264920.1 | type II CRISPR RNA-guided endonuclease Cas9 [Streptococcus mutans] SEQ ID NO: 125 | | |
| WP_002269043.1 | type II CRISPR RNA-guided endonuclease Cas9 [Streptococcus mutans] SEQ ID NO: 126 | | |
| WP_002269448.1 | type II CRISPR RNA-guided endonuclease Cas9 [Streptococcus mutans] SEQ ID NO: 127 | | |
| WP_002271977.1 | type II CRISPR RNA-guided endonuclease Cas9 [Streptococcus mutans] SEQ ID NO: 128 | | |
| WP_002272766.1 | type II CRISPR RNA-guided endonuclease Cas9 [Streptococcus mutans] SEQ ID NO: 129 | | |
| WP_002273241.1 | type II CRISPR RNA-guided endonuclease Cas9 [Streptococcus mutans] SEQ ID NO: 130 | | |
| WP_002275430.1 | type II CRISPR RNA-guided endonuclease Cas9 [Streptococcus mutans] SEQ ID NO: 131 | | |
| WP_002276448.1 | type II CRISPR RNA-guided endonuclease Cas9 [Streptococcus mutans] SEQ ID NO: 132 | | |
| WP_002277050.1 | type II CRISPR RNA-guided endonuclease Cas9 [Streptococcus mutans] SEQ ID NO: 133 | | |
| WP_002277364.1 | type II CRISPR RNA-guided endonuclease Cas9 [Streptococcus mutans] SEQ ID NO: 134 | | |
| WP_002279025.1 | type II CRISPR RNA-guided endonuclease Cas9 [Streptococcus mutans] SEQ ID NO: 135 | | |
| WP_002279859.1 | type II CRISPR RNA-guided endonuclease Cas9 [Streptococcus mutans] SEQ ID NO: 136 | | |
| WP_002280230.1 | type II CRISPR RNA-guided endonuclease Cas9 [Streptococcus mutans] SEQ ID NO: 137 | | |
| WP_002281696.1 | type II CRISPR RNA-guided endonuclease Cas9 [Streptococcus mutans] SEQ ID NO: 138 | | |
| WP_002282247.1 | type II CRISPR RNA-guided endonuclease Cas9 [Streptococcus mutans] SEQ ID NO: 139 | | |
| WP_002282906.1 | type II CRISPR RNA-guided endonuclease Cas9 [Streptococcus mutans] SEQ ID NO: 140 | | |
| WP_002283846.1 | type II CRISPR RNA-guided endonuclease Cas9 [Streptococcus mutans] SEQ ID NO: 141 | | |
| WP_002287255.1 | type II CRISPR RNA-guided endonuclease Cas9 [Streptococcus mutans] SEQ ID NO: 142 | | |
| WP_002288990.1 | type II CRISPR RNA-guided endonuclease Cas9 [Streptococcus mutans] SEQ ID NO: 143 | | |
| WP_002289641.1 | type II CRISPR RNA-guided endonuclease Cas9 [Streptococcus mutans] SEQ ID NO: 144 | | |
| WP_002290427.1 | type II CRISPR RNA-guided endonuclease Cas9 [Streptococcus mutans] SEQ ID NO: 145 | | |
| WP_002295753.1 | type II CRISPR RNA-guided endonuclease Cas9 [Streptococcus mutans] SEQ ID NO: 146 | | |
| WP_002296423.1 | type II CRISPR RNA-guided endonuclease Cas9 [Streptococcus mutans] SEQ ID NO: 147 | | |
| WP_002304487.1 | type II CRISPR RNA-guided endonuclease Cas9 [Streptococcus mutans] SEQ ID NO: 148 | | |
| WP_002305844.1 | type II CRISPR RNA-guided endonuclease Cas9 [Streptococcus mutans] SEQ ID NO: 149 | | |
| WP_002307203.1 | type II CRISPR RNA-guided endonuclease Cas9 [Streptococcus mutans] SEQ ID NO: 150 | | |
| WP_002310390.1 | type II CRISPR RNA-guided endonuclease Cas9 [Streptococcus mutans] SEQ ID NO: 151 | | |
| WP_002352408.1 | type II CRISPR RNA-guided endonuclease Cas9 [Streptococcus mutans] SEQ ID NO: 152 | | |
| WP_012997688.1 | type II CRISPR RNA-guided endonuclease Cas9 [Streptococcus mutans] SEQ ID NO: 153 | | |
| WP_014677909.1 | type II CRISPR RNA-guided endonuclease Cas9 [Streptococcus mutans] SEQ ID NO: 154 | | |
| WP_019312892.1 | type II CRISPR RNA-guided endonuclease Cas9 [Streptococcus mutans] SEQ ID NO: 155 | | |
| WP_019313659.1 | type II CRISPR RNA-guided endonuclease Cas9 [Streptococcus mutans] SEQ ID NO: 156 | | |
| WP_019314093.1 | type II CRISPR RNA-guided endonuclease Cas9 [Streptococcus mutans] SEQ ID NO: 157 | | |
| WP_019315370.1 | type II CRISPR RNA-guided endonuclease Cas9 [Streptococcus mutans] SEQ ID NO: 158 | | |
| WP_019803776.1 | type II CRISPR RNA-guided endonuclease Cas9 [Streptococcus mutans] SEQ ID NO: 159 | | |
| WP_019805234.1 | type II CRISPR RNA-guided endonuclease Cas9 [Streptococcus mutans] SEQ ID NO: 160 | | |
| WP_024783594.1 | type II CRISPR RNA-guided endonuclease Cas9 [Streptococcus mutans] SEQ ID NO: 161 | | |
| WP_024784288.1 | type II CRISPR RNA-guided endonuclease Cas9 [Streptococcus mutans] SEQ ID NO: 162 | | |
| WP_024784666.1 | type II CRISPR RNA-guided endonuclease Cas9 [Streptococcus mutans] SEQ ID NO: 163 | | |
| WP_024784894.1 | type II CRISPR RNA-guided endonuclease Cas9 [Streptococcus mutans] SEQ ID NO: 164 | | |
| WP_024786433.1 | type II CRISPR RNA-guided endonuclease Cas9 [Streptococcus mutans] SEQ ID NO: 165 | | |
| WP_049473442.1 | CRISPR-associated protein Csn1 [Streptococcus mutans] SEQ ID NO: 166 | | |
| WP_049474547.1 | CRISPR-associated protein Csn1 [Streptococcus mutans] SEQ ID NO: 167 | | |
| EMC03581.1 | hypothetical protein SMU69_09359 [Streptococcus mutans NLML4] SEQ ID NO: 168 | | |
| WP_000428612.1 | type II CRISPR RNA-guided endonuclease Cas9 [Streptococcus oralis] SEQ ID NO: 169 | | |
| WP_000428613.1 | type II CRISPR RNA-guided endonuclease Cas9 [Streptococcus oralis] SEQ ID NO: 170 | | |
| WP_049523028.1 | CRISPR-associated protein Csn1 [Streptococcus parasanguinis] SEQ ID NO: 171 | | |
| WP_003107102.1 | type II CRISPR RNA-guided endonuclease Cas9 [Streptococcus parauberis] SEQ ID NO: 172 | | |
| WP_054279288.1 | type II CRISPR RNA-guided endonuclease Cas9 [Streptococcus phocae] SEQ ID NO: 173 | | |
| WP_049531101.1 | CRISPR-associated protein Csn1 [Streptococcus pseudopneumoniae] SEQ ID NO: 174 | | |
| WP_049538452.1 | CRISPR-associated protein Csn1 [Streptococcus pseudopneumoniae] SEQ ID NO: 175 | | |
| WP_049549711.1 | CRISPR-associated protein Csn1 [Streptococcus pseudopneumoniae] SEQ ID NO: 176 | | |
| WP_007896501.1 | type II CRISPR RNA-guided endonuclease Cas9 [Streptococcus pseudoporcinus] SEQ ID NO: 177 | | |
| EFR44625.1 | CRISPR-associated protein, Csn1 family [Streptococcus pseudoporcinus SPIN 20026] SEQ ID NO: 178 | | |
| WP_002897477.1 | type II CRISPR RNA-guided endonuclease Cas9 [Streptococcus sanguinis] SEQ ID NO: 179 | | |
| WP_002906454.1 | type II CRISPR RNA-guided endonuclease Cas9 [Streptococcus sanguinis] SEQ ID NO: 180 | | |
| WP_009729476.1 | type II CRISPR RNA-guided endonuclease Cas9 [Streptococcus sp. F0441] SEQ ID NO: 181 | | |
| CQR24647.1 | CRISPR-associated protein [Streptococcus sp. FF10] SEQ ID NO: 182 | | |
| WP_000066813.1 | type II CRISPR RNA-guided endonuclease Cas9 [Streptococcus sp. M334] SEQ ID NO: 183 | | |
| WP_009754323.1 | type II CRISPR RNA-guided endonuclease Cas9 [Streptococcus sp. taxon 056] SEQ ID NO: 184 | | |
| WP_044674937.1 | type II CRISPR RNA-guided endonuclease Cas9 [Streptococcus suis] SEQ ID NO: 185 | | |
| WP_044676715.1 | type II CRISPR RNA-guided endonuclease Cas9 [Streptococcus suis] SEQ ID NO: 186 | | |
| WP_044680361.1 | type II CRISPR RNA-guided endonuclease Cas9 [Streptococcus suis] SEQ ID NO: 187 | | |
| WP_044681799.1 | type II CRISPR RNA-guided endonuclease Cas9 [Streptococcus suis] SEQ ID NO: 188 | | |
| WP_049533112.1 | CRISPR-associated protein Csn1 [Streptococcus suis] SEQ ID NO: 189 | | |
| WP_029090905.1 | type II CRISPR RNA-guided endonuclease Cas9 [Brochothrix thermosphacta] SEQ ID NO: 190 | | |
| WP_006506696.1 | type II CRISPR RNA-guided endonuclease Cas9 [Catenibacterium mitsuokai] SEQ ID NO: 191 | | |
| AIT42264.1 | Cas9hc : NLS : HA [Cloning vector pYB196] SEQ ID NO: 192 | | |
| WP_034440723.1 | type II CRISPR endonuclease Cas9 [Clostridiales bacterium S5-A11] SEQ ID NO: 193 | | |
| AKQ21048.1 | Cas9 [CRISPR-mediated gene targeting vector p (bhsp68-Cas9) ] SEQ ID NO: 194 | | |
| WP_004636532.1 | type II CRISPR RNA-guided endonuclease Cas9 [Dolosigranulum pigrum] SEQ ID NO: 195 | | |
| WP_002364836.1 | MULTISPECIES: type II CRISPR RNA-guided endonuclease Cas9 [Enterococcus] SEQ ID NO: 196 | | |
| WP_016631044.1 | MULTISPECIES: type II CRISPR RNA-guided endonuclease Cas9 [Enterococcus] SEQ ID NO: 197 | | |
| EMS75795.1 | hypothetical protein H318_06676 [Enterococcus durans IPLA 655] SEQ ID NO: 198 | | |
| WP_002373311.1 | type II CRISPR RNA-guided endonuclease Cas9 [Enterococcus faecalis] SEQ ID NO: 199 | | |
| WP_002378009.1 | type II CRISPR RNA-guided endonuclease Cas9 [Enterococcus faecalis] SEQ ID NO: 200 | | |
| WP_002407324.1 | type II CRISPR RNA-guided endonuclease Cas9 [Enterococcus faecalis] SEQ ID NO: 201 | | |
| WP_002413717.1 | type II CRISPR RNA-guided endonuclease Cas9 [Enterococcus faecalis] SEQ ID NO: 202 | | |
| WP_010775580.1 | type II CRISPR RNA-guided endonuclease Cas9 [Enterococcus faecalis] SEQ ID NO: 203 | | |
| WP_010818269.1 | type II CRISPR RNA-guided endonuclease Cas9 [Enterococcus faecalis] SEQ ID NO: 204 | | |
| WP_010824395.1 | type II CRISPR RNA-guided endonuclease Cas9 [Enterococcus faecalis] SEQ ID NO: 205 | | |
| WP_016622645.1 | type II CRISPR RNA-guided endonuclease Cas9 [Enterococcus faecalis] SEQ ID NO: 206 | | |
| WP_033624816.1 | type II CRISPR RNA-guided endonuclease Cas9 [Enterococcus faecalis] SEQ ID NO: 207 | | |
| WP_033625576.1 | type II CRISPR RNA-guided endonuclease Cas9 [Enterococcus faecalis] SEQ ID NO: 208 | | |
| WP_033789179.1 | type II CRISPR RNA-guided endonuclease Cas9 [Enterococcus faecalis] SEQ ID NO: 209 | | |
| WP_002310644.1 | type II CRISPR RNA-guided endonuclease Cas9 [Enterococcus faecium] SEQ ID NO: 210 | | |
| WP_002312694.1 | type II CRISPR RNA-guided endonuclease Cas9 [Enterococcus faecium] SEQ ID NO: 211 | | |
| WP_002314015.1 | type II CRISPR RNA-guided endonuclease Cas9 [Enterococcus faecium] SEQ ID NO: 212 | | |
| WP_002320716.1 | type II CRISPR RNA-guided endonuclease Cas9 [Enterococcus faecium] SEQ ID NO: 213 | | |
| WP_002330729.1 | type II CRISPR RNA-guided endonuclease Cas9 [Enterococcus faecium] SEQ ID NO: 214 | | |
| WP_002335161.1 | type II CRISPR RNA-guided endonuclease Cas9 [Enterococcus faecium] SEQ ID NO: 215 | | |
| WP_002345439.1 | type II CRISPR RNA-guided endonuclease Cas9 [Enterococcus faecium] SEQ ID NO: 216 | | |
| WP_034867970.1 | type II CRISPR RNA-guided endonuclease Cas9 [Enterococcus faecium] SEQ ID NO: 217 | | |
| WP_047937432.1 | type II CRISPR RNA-guided endonuclease Cas9 [Enterococcus faecium] SEQ ID NO: 218 | | |
| WP_010720994.1 | type II CRISPR RNA-guided endonuclease Cas9 [Enterococcus hirae] SEQ ID NO: 219 | | |
| WP_010737004.1 | type II CRISPR RNA-guided endonuclease Cas9 [Enterococcus hirae] SEQ ID NO: 220 | | |
| WP_034700478.1 | type II CRISPR RNA-guided endonuclease Cas9 [Enterococcus hirae] SEQ ID NO: 221 | | |
| WP_007209003.1 | type II CRISPR RNA-guided endonuclease Cas9 [Enterococcus italicus] SEQ ID NO: 222 | | |
| WP_023519017.1 | type II CRISPR RNA-guided endonuclease Cas9 [Enterococcus mundtii] SEQ ID NO: 223 | | |
| WP_010770040.1 | type II CRISPR RNA-guided endonuclease Cas9 [Enterococcus phoeniculicola] SEQ ID NO: 224 | | |
| WP_048604708.1 | type II CRISPR RNA-guided endonuclease Cas9 [Enterococcus sp. AM1] SEQ ID NO: 225 | | |
| WP_010750235.1 | type II CRISPR RNA-guided endonuclease Cas9 [Enterococcus villorum] SEQ ID NO: 226 | | |
| AII16583.1 | Cas9 endonuclease [Expression vector pCas9] SEQ ID NO: 227 | | |
| WP_029073316.1 | type II CRISPR RNA-guided endonuclease Cas9 [Kandleria vitulina] SEQ ID NO: 228 | | |
| WP_031589969.1 | type II CRISPR RNA-guided endonuclease Cas9 [Kandleria vitulina] SEQ ID NO: 229 | | |
| KDA45870.1 | CRISPR-associated protein Cas9/Csn1, subtype II/NMEMI [Lactobacillus animalis] SEQ ID NO: 230 | | |
| WP_039099354.1 | type II CRISPR RNA-guided endonuclease Cas9 [Lactobacillus curvatus] SEQ ID NO: 231 | | |
| AKP02966.1 | hypothetical protein ABB45_04605 [Lactobacillus farciminis] SEQ ID NO: 232 | | |
| WP_010991369.1 | type II CRISPR RNA-guided endonuclease Cas9 [Listeria innocua] SEQ ID NO: 233 | | |
| WP_033838504.1 | type II CRISPR RNA-guided endonuclease Cas9 [Listeria innocua] SEQ ID NO: 234 | | |
| EHN60060.1 | CRISPR-associated protein, Csn1 family [Listeria innocua ATCC 33091] SEQ ID NO: 235 | | |
| EFR89594.1 | crispr-associated protein, Csn1 family [Listeria innocua FSL S4-378] SEQ ID NO: 236 | | |
| WP_038409211.1 | type II CRISPR RNA-guided endonuclease Cas9 [Listeria ivanovii] SEQ ID NO: 237 | | |
| EFR95520.1 | crispr-associated protein Csn1 [Listeria ivanovii FSL F6-596] SEQ ID NO: 238 | | |
| WP_003723650.1 | type II CRISPR RNA-guided endonuclease Cas9 [Listeria monocytogenes] SEQ ID NO: 239 | | |
| WP_003727705.1 | type II CRISPR RNA-guided endonuclease Cas9 [Listeria monocytogenes] SEQ ID NO: 240 | | |
| WP_003730785.1 | type II CRISPR RNA-guided endonuclease Cas9 [Listeria monocytogenes] SEQ ID NO: 241 | | |
| WP_003733029.1 | type II CRISPR RNA-guided endonuclease Cas9 [Listeria monocytogenes] SEQ ID NO: 242 | | |
| WP_003739838.1 | type II CRISPR RNA-guided endonuclease Cas9 [Listeria monocytogenes] SEQ ID NO: 243 | | |
| WP_014601172.1 | type II CRISPR RNA-guided endonuclease Cas9 [Listeria monocytogenes] SEQ ID NO: 244 | | |
| WP_023548323.1 | type II CRISPR RNA-guided endonuclease Cas9 [Listeria monocytogenes] SEQ ID NO: 245 | | |
| WP_031665337.1 | type II CRISPR RNA-guided endonuclease Cas9 [Listeria monocytogenes] SEQ ID NO: 246 | | |
| WP_031669209.1 | type II CRISPR RNA-guided endonuclease Cas9 [Listeria monocytogenes] SEQ ID NO: 247 | | |
| WP_033920898.1 | type II CRISPR RNA-guided endonuclease Cas9 [Listeria monocytogenes] SEQ ID NO: 248 | | |
| AKI42028.1 | CRISPR-associated protein [Listeria monocytogenes] SEQ ID NO: 249 | | |
| AKI50529.1 | CRISPR-associated protein [Listeria monocytogenes] SEQ ID NO: 250 | | |
| EFR83390.1 | crispr-associated protein Csn1 [Listeria monocytogenes FSL F2-208] SEQ ID NO: 251 | | |
| WP_046323366.1 | type II CRISPR RNA-guided endonuclease Cas9 [Listeria seeligeri] SEQ ID NO: 252 | | |
| AKE81011.1 | Cas9 [Plant multiplex genome editing vector pYLCRISPR/Cas9Pubi-H] SEQ ID NO: 253 | | |
| CUO82355.1 | Uncharacterized protein conserved in bacteria [Roseburia hominis] SEQ ID NO: 254 | | |
| WP_033162887.1 | type II CRISPR RNA-guided endonuclease Cas9 [Sharpea azabuensis] SEQ ID NO: 255 | | |
| AGZ01981.1 | Cas9 endonuclease [synthetic construct] SEQ ID NO: 256 | | |
| AKA60242.1 | nuclease deficient Cas9 [synthetic construct] SEQ ID NO: 257 | | |
| AKS40380.1 | Cas9 [Synthetic plasmid pFC330] SEQ ID NO: 258 | | |
| 4UN5_B | Cas9, Chain B, Crystal Structure SEQ ID NO: 259 | | |
| WP_010922251 | 1 | 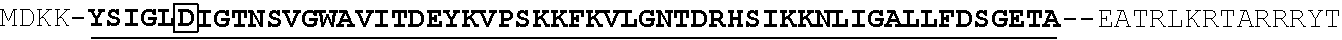 | 73 |
| WP_039695303 | 1 | MTKKnYSIGLDIGTNSVGWAVITDDYKVPAKKMKVLGNTDKKYIKKNLLGALLFDSGETA--EATRLKRTARRRYT | 74 |
| WP_045635197 | 1 | K-KG-YSIGLDIGTNSVGFAVITDDYKVPSKKMKVLGNTDKRFIKKNLIGALLFDEGTTA--EARRLKRTARRRYT | 73 |
| 5AXW_A | 1 | MKRN-YILGLDIGITSVGYGII--DYET------------RDVIDA---GVRLFKEANVEnnEGRRSKRGARRLKR | 61 |
| WP_009880683 | | --------------------------------------------------------------------------- | |
| WP_010922251 | 1 | MDKK-YSIGLDIGTNSVGWAVITDEYKVPSKKFKVLGNTDRHSIKKNLIGALLFDSGETA--EATRLKRTARRRYT | 73 |
| WP_011054416 | 1 | MDKK-YSIGLDIGTNSVGWAVITDDYKVPSKKLKGLGNTDRHGIKKNLIGALLFDSGETA--EATRLKRTARRRYT | 73 |
| WP_011284745 | 1 | MDKK-YSIGLDIGTNSVGWAVITDDYKVPSKKFKVLGNTDRHSIKKNLIGALLFDSGETA--EATRLKRTARRRYT | 73 |
| WP_011285506 | 1 | MDKK-YSIGLDIGTNSVGWAVITDDYKVPSKKFKVLGNTDRHSIKKNLIGALLFDSGETA--EATRLKRTARRRYT | 73 |
| WP_011527619 | 1 | MDKK-YSIGLDIGTNSVGWAVITDDYKVPSKKFKVLGNTDRHSIKKNLIGALLFDSGEIA--EATRLKRTARRRYT | 73 |
| WP_012560673 | 1 | MDKK-YSIGLDIGTNSVGWAVITDDYKVPSKKFKVLGNTDRHSIKKNLIGALLFDSGETA--EATRLKRTARRRYT | 73 |
| WP_014407541 | 1 | MDKK-YSIGLDIGTNSVGWAVITDDYKVPSKKFKVLGNTDRHSIKKNLIGALLFGSGETA--EATRLKRTARRRYT | 73 |
| WP_020905136 | 1 | MDKK-YSIGLDIGTNSVGWAVITDDYKVPSKKFKVLGNTDRHSIKKNLIGALLFDSGETA--EATRLKRTARRRYT | 73 |
| WP_023080005 | 1 | MDKK-YSIGLDIGTNSVGWAVITDDYKVPSKKLKVLGNTDRHGIKKNLIGALLFDSGETA--EATRLKRTARRRYT | 73 |
| WP_023610282 | 1 | MDKK-YSIGLDIGTNSVGWAVITDDYKVPSKKLKVLGNTDRHGIKKNLIGALLFDSGETA--EATRLKRTARRRYT | 73 |
| WP_030125963 | 1 | MDKK-YSIGLDIGTNSVGWAVITDDYKVPSKKFKVLGNTDRHSIKKNLIGALLFDSGETA--EATRLKRTARRRYT | 73 |
| WP_030126706 | 1 | MDKK-YSIGLDIGTNSVGWAVITDDYKVPSKKFKVLGNTDRHGIKKNLIGALLFDSGETA--EATRLKRTARRRYT | 73 |
| WP_031488318 | 1 | MDKK-YSIGLDIGTNSVGWAVITDDYKVPSKKFKVLGNTDRHSIKKNLIGALLFDSGETA--EATRLKRTARRRYT | 73 |
| WP_032460140 | 1 | MDKK-YSIGLDIGTNSVGWAVITDDYKVPSKKFKVLGNTDRHSIKKNLIGALLFDSGETA--EATRLKRTARRRYT | 73 |
| WP_032461047 | 1 | MDKK-YSIGLDIGTNSVGWAVITDDYKVPSKKFKVLGNTDRHSIKKNLIGALLFDSGETA--EATRLKRTARRRYT | 73 |
| WP_032462016 | 1 | MDKK-YSIGLDIGTNSVGWAVITDDYKVPSKKFKVLGNTDRHSIKKNLIGALLFDSGETA--EATRLKRTARRRYT | 73 |
| WP_032462936 | 1 | MDKK-YSIGLDIGTNSVGWAVITDDYKVPSKKFKVLGNTDRHSIKKNLIGALLFDSGETA--EATRLKRTARRRYT | 73 |
| WP_032464890 | 1 | MDKK-YSIGLDIGTNSVGWAVITDDYKVPSKKFKVLGNTDRHSIKKNLIGALLFDSGEIA--EATRLKRTARRRYT | 73 |
| WP_033888930 | | --------------------------------------------------------------------------- | |
| WP_038431314 | 1 | MDKK-YSIGLDIGTNSVGWAVITDDYKVPSKKFKVLGNTDRHSIKKNLIGALLFDSGETA--EATRLKRTARRRYT | 73 |
| WP_038432938 | 1 | MDKK-YSIGLDIGTNSVGWAVITDDYKVPSKKFKVLGNTDRHSIKKNLIGALLFDSGETA--EATRLKRTARRRYT | 73 |
| WP_038434062 | 1 | MDKK-YSIGLDIGTNSVGWAVITDDYKVPSKKFKVLGNTDRHSIKKNLIGALLFDSGETA--EATRLKRTARRRYT | 73 |
| BAQ51233 | | --------------------------------------------------------------------------- | |
| KGE60162 | | --------------------------------------------------------------------------- | |
| KGE60856 | | --------------------------------------------------------------------------- | |
| WP_002989955 | 1 | MDKK-YSIGLDIGTNSVGWAVITDDYKVPSKKFKVLGNTDRHSIKKNLIGALLFDSGEIA--EATRLKRTARRRYT | 73 |
| WP_003030002 | 1 | MDQK-YSIGLDIGTNSVGWAVVTDDYKVPAKKMKVLGNTDKQSIKKNLLGALLFDSGETA--EATRLKRTARRRYT | 73 |
| WP_003065552 | 1 | MTKKNYSIGLDIGTNSVGWAVITDDYKVPAKKMKVLGNTDKKYIKKNLLGALLFDSGETA--EATRLKRTARRRYT | 74 |
| WP_001040076 | 1 | MNKP-YSIGLDIGTNSVGWSIITDDYKVPAKKIRVLGNTDKEYIKKNLIGALLFDGGNTA--ADRRLKRTARRRYT | 73 |
| WP_001040078 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040080 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040081 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040083 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040085 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040087 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040088 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040089 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040090 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040091 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040092 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040094 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040095 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040096 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040097 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040098 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040099 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040100 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040104 | 1 | MNKP-YSIGLDIGTNSVGWSIITDDYKVPAKKMRVLGNTDKEYIKKNLIGALLFDGGNTA--ADRRLKRTARRRYT | 73 |
| WP_001040105 | 1 | MNKP-YSIGLDIGTNSVGWSIITDDYKVPAKKMRVLGNTDKEYIKKNLIGALLFDGGNTA--ADRRLKRTSRRRYT | 73 |
| WP_001040106 | 1 | MNKP-YSIGLDIGTNSVGYSVVTDDYKVPAKKMRVLGNTDKEYIKKNLIGALLFDGGNTA--SDRRLKRTARRRYT | 73 |
| WP_001040107 | 1 | MNKP-YSIGLDIGTNSVGYSVVTDDYKVPAKKMRVLGNTDKEYIKKNLIGALLFDGGNTA--SDRRLKRTARRRYT | 73 |
| WP_001040108 | 1 | MNKP-YSIGLDIGTNSVGYSVVTDDYKVPAKKMRVLGNTDKEYIKKNLIGALLFDGGNTA--SDRRLKRTARRRYT | 73 |
| WP_001040109 | 1 | MNKP-YSIGLDIGTNSVGYSVVTDDYKVPAKKMRVLGNTDKEYIKKNLIGALLFDGGNTA--SDRRLKRTARRRYT | 73 |
| WP_001040110 | 1 | MNKP-YSIGLDIGTNSVGYSVVTDDYKVPAKKMRVLGNTDKEYIKKNLIGALLFDGGNTA--SDRRLKRTARRRYT | 73 |
| WP_015058523 | 1 | MNKP-YSIGLDIGTNSVGWSIITDDYKVPAKKMRVLGNTDKEYIKKNLIGALLFDGGNTA--ADRRLKRTARRRYT | 73 |
| WP_017643650 | 1 | MNKP-YSIGLDIGTNSVGWSIITDDYKVPAKKMRVLGNTDKEYIKKNLIGALLFDGGNTA--ADRRLKRTARRRYT | 73 |
| WP_017647151 | 1 | MNKP-YSIGLDIGTNSVGWSIITDDYKVPAKKMRVLGNTDKEYIKKNLIGALLFDGGNTA--SDRRLKRTARRRYT | 73 |
| WP_017648376 | 1 | MNKP-YSIGLDIGTNSVGWSIITDDYKVPAKKMRVLGNTDKEYIKKNLIGALLFDGGNTA--SDRRLKRTARRRYT | 73 |
| WP_017649527 | 1 | MNKP-YSIGLDIGTNSVGWSIITDDYKVPAKKMRVLGNTDKEYIKKNLIGALLFDGGNTA--ADRRLKRTARRRYT | 73 |
| WP_017771611 | 1 | MNKP-YSIGLDIGTNSVGYSVVTDDYKVPAKKMRVLGNTDKEYIKKNLIGALLFDGGNTA--SDRRLKRTARRRYT | 73 |
| WP_017771984 | 1 | MNKP-YSIGLDIGTNSVGWSIITDDYKVPAKKMRVLGNTDKEYIKKNLIGALLFDGGNTA--ADRRLKRTARRRYT | 73 |
| CFQ25032 | 1 | MNKP-YSIGLDIGTNSVGWSIITDDYKVPAKKMRVLGNTDKEYIKKNLIGALLFDGGNTA--ADRRLKRTARRRYT | 73 |
| CFV16040 | 1 | MNKP-YSIGLDIGTNSVGWSIITDDYKVPAKKMRVLGNTDKEYIKKNLIGALLFDGGNTA--ADRRLKRTARRRYT | 73 |
| KLJ37842 | 1 | MNKP-YSIGLDIGTNSVGWSIITDDYKVPAKKMRVLGNTDKEYIKKNLIGALLFDGGNTA--ADRRLKRTARRRYT | 73 |
| KLJ72361 | 1 | MNKP-YSIGLDIGTNSVGWSIITDDYKVPAKKMRVLGNTDKEYIKKNLIGALLFDGGNTA--ADRRLKRTARRRYT | 73 |
| KLL20707 | 1 | MNKP-YSIGLDIGTNSVGWSIITDDYKVPAKKMRVLGNTDKEYIKKNLIGALLFDGGNTA--ADRRLKRTARRRYT | 73 |
| KLL42645 | 1 | MNKP-YSIGLDIGTNSVGYSVVTDDYKVPAKKMRVLGNTDKEYIKKNLIGALLFDGGNTA--SDRRLKRTARRRYT | 73 |
| WP_047207273 | 1 | MNKP-YSIGLDIGTNSVGWSIITDDYKVPAKKMRVLGNTDKEYIKKNLIGALLFDGRNTA--ADRRLKRTARRRYT | 73 |
| WP_047209694 | 1 | MNKP-YSIGLDIGTNSVGWSIITDDYKVPAKKMRVLGNTDKEYIKKNLIGALLFDGGNTA--ADRRLKRTARRRYT | 73 |
| WP_050198062 | 1 | MNKP-YSIGLDIGTNSVGWSIITDDYKVPAKKMRVLGNTDKEYIKKNLIGALLFDGGNTA--ADRRLKRTARRRYT | 73 |
| WP_050201642 | 1 | MNKP-YSIGLDIGTNSVGWSIITDDYKVPAKKMRVLGNTDKEYIKKNLIGALLFDGGNTA--ADRRLKRTARRRYT | 73 |
| WP_050204027 | 1 | MNKP-YSIGLDIGTNSVGYSVVTDDYKVPAKKMRVLGNTDKEYIKKNLIGALLFDGGNTA--SDRRLKRTARRRYT | 73 |
| WP_050881965 | 1 | MNKP-YSIGLDIGTNSVGWSIITDDYKVPAKKMRVLGNTDKEYIKKNLIGALLFDGGNTA--ADRRLKRTARRRYT | 73 |
| WP_050886065 | 1 | MNKP-YSIGLDIGTNSVGWSIITDDYKVPAKKMRVLGNTDKEYIKKNLIGALLFDGGNTA--ADRRLKRTARRRYT | 73 |
| AHN30376 | 1 | MNKP-YSIGLDIGTNSVGWSIITDDYKVPAKKMRVLGNTDKEYIKKNLIGALLFDGGNTA--ADRRLKRTARRRYT | 73 |
| EAO78426 | 1 | MNKP-YSIGXDIGTNSVGWSIITDDYKVPAKKMRVLGNTDKEYIKKNLIGALLFDGGNTA--ADRRLKRTARRRYT | 73 |
| CCW42055 | 1 | MNKP-YSIGLDIGTNSVGWSIITDDYKVPAKKMRVLGNTDKEYIKKNLIGALLFDGGNTA--ADRRLKRIARRRYT | 73 |
| WP_003041502 | 1 | MNQK-YSIGLDIGTNSVGWAVITDDYKVPAKKMKVLGNTDKQSIKKNLLGALLFDSGETA--EATRLKRTARRRYT | 73 |
| WP_037593752 | 1 | MKKE-YSIGLDIGTNSVGWAVITDDYKVPAKKMKVLGNTDKQSIKKNLLGALLFDSGETA--EATRLKRTARRRYT | 74 |
| WP_049516684 | 1 | MKKE-YSIGLDIGTNSVGWAVITDDYKVPAKKMKVLGNTDKQSIKKNLLGALLFDSGETA--EATRLKRTARRRYT | 74 |
| GAD46167 | 1 | MKKE-YSIGLDIGTNSVGWAVITDDYKVPAKKMKVLGNTDKQSIKKNLLGALLFDSGETA--EATRLKRTARRRYT | 73 |
| WP_018363470 | 1 | MTKKnYSIGLDIGTNSVGWAVITDDYKVPAKKMKVLGNTDKKYIKKNLLGALLFDSGETA--EATRLKRTARRRYT | 74 |
| WP_003043819 | 1 | MEKK-YSIGLDIGTNSVGWAVITDDYKVPSKKFKVLGNTNRKSIKKNLMGALLFDSGETA--EATRLKRTARRRYT | 73 |
| WP_006269658 | 1 | MGKP-YSIGLDIGTNSVGWAVVTDDYKVPAKKMKVLGNTDKQSIKKNLLGALLFDSGETA--EATRLKRTARRRYT | 73 |
| WP_048800889 | 1 | MTQK-YSIGLDIGTNSVGWAIVIDDYKVPAKKMKILGNTNKQYIKKNLLGALLFDSGETA--KATRLKRTARRRYT | 73 |
| WP_012767106 | 1 | MDKK-YSIGLDIGTNSVGWAVITDDYKVPSKKFKVLGNTDRHSIKKNLIGALLFDSGETA--EATRLKRTARRRYT | 73 |
| WP_014612333 | 1 | MDKK-YSIGLDIGTNSVGWAVITDDYKVPSKKFKVLGNTDRHSIKKNLIGALLFDSGETA--EATRLKRTARRRYT | 73 |
| WP_015017095 | 1 | MDKK-YSIGLDIGTNSVGWAVITDDYKVPSKKFKVLGNTDRHSIKKNLIGALLFDSGETA--EATRLKRTARRRYT | 73 |
| WP_015057649 | 1 | MDKK-YSIGLDIGTNSVGWAVITDDYKVPSKKFKVLGNTDRHSIKKNLIGALLFDSGETA--EATRLKRTARRRYT | 73 |
| WP_048327215 | 1 | MDKK-YSIGLDIGTNSVGWAVITDDYKVPSKKFKVLGNTDRHSIKKNLIGALLFDSGETA--EATRLKRTARRRYT | 73 |
| WP_049519324 | 1 | MDKK-YSIGLDIGTNSVGWAVITDDYKVPSKKFKVLGNTDRHSIKKNLIGALLFDSGETA--EATRLKRTARRRYT | 73 |
| WP_012515931 | 1 | MKKP-YTIALDIGTNSVGWVVVTDDYRVPTKKMKVLGNTERKTIKKNLIGALLFDSGDTA--EGTRLKRTARRRYT | 73 |
| WP_021320964 | 1 | MKKP-YTIALDIGTNSVGWVVVTDDYRVPTKKMKVLGNTERKTIKKNLIGALLFDSGDTA--EGTRLKRTARRRYT | 73 |
| WP_037581760 | 1 | MKKP-YTIALDIGTNSVGWVVVTDDYRVPTKKMKVLGNTERKTIKKNLIGALLFDSGDTA--EGTRLKRTARPRYT | 73 |
| WP_004232481 | 1 | M-EKtYSIGLDIGTNSVGWAVITDDYKVPAKKMKVLGNTDKKYIKKNLLGALLFDSGETA--EATRLKRAARRRYT | 73 |
| WP_009854540 | 1 | MTKKNYSIGLDIGTNSVGWAVITDDYKVPAKKMKVLGNTDKKYIKKNLLGALLFDSGETA--EATRLKRTARRRYT | 74 |
| WP_012962174 | 1 | MTEKnYSIGLDIGTNSVGWAVITDDYKVPAKKMKVLGNTDKKYIKKNLLGALLFDNGETA--EATRLKRTARRRYT | 74 |
| WP_039695303 | 1 | MTKKNYSIGLDIGTNSVGWAVITDDYKVPAKKMKVLGNTDKKYIKKNLLGALLFDSGETA--EATRLKRTARRRYT | 74 |
| WP_014334983 | 1 | M-EKSYSIGLDIGTNSVGWAVITDDYKVPAKKMKVLGNTDKKYIKKNLLGALLFDSGETA--EVTRLKRTARRRYT | 73 |
| WP_003099269 | 1 | MRKP-YSIGLDIGTNSVGWAVITDDYKVPSKKMRIQGTTDRTSIKKNLIGALLFDNGETA--EATRLKRITRRRYT | 73 |
| AHY15608 | 1 | MRKP-YSIGLDIGTNSVGWAVITDDYKVPSKKMRIQGTTDRTSIKKNLIGALLFDNGETA--EATRLKRTTRRRYT | 73 |
| AHY17476 | 1 | MRKP-YSIGLDIGTNSVGWAVITDDYKVPSKKMRIQGTTDRTSIKKNLIGALLFDNGETA--EATRLKRTTRRRYT | 73 |
| ESR09100 | | --------------------------------------------------------------------------- | |
| AGM98575 | 1 | MRKP-YSIGLDIGTNSVGWAVITDDYKVPSKKMRIQGTTDRTSIKKNLIGALLFDNGETA--EATRLKRTTRRRYT | 73 |
| ALF27331 | 1 | MKKP-YSIGLDIGTNSVGWAVVTDDYKVPAKKMKVLGNTDKSHIKKNLLGALLFDSGNTA--EDRRLKRTARRRYT | 73 |
| WP_018372492 | 1 | MKKP-YSIGLDIGTNSVGWAVVMEDYKVPSKKMKVLGNTDKQSIKKNLIGALLFDSGETAV--ERRLNRTTSRRYD | 73 |
| WP_045618028 | 1 | NNKP-YSIGLDIGTNSVGWAVITDDYKVPSKKMKVLGNTDKHFIKKNLLGALLFDEGTTA--EDRRLKRTARRRYT | 74 |
| WP_045635197 | 1 | K-KG-YSIGLDIGTNSVGFAVITDDYKVPSKKMKVLGNTDKRFIKKNLIGALLFDEGTTA--EARRLKRTARRRYT | 73 |
| WP_002263549 | 1 | MKKP-YSIGLDIGTNSVGWAVVTDDYKVPAKKMKVLGNTDKSHIEKNLLGALLFDSGNTA--EDRRLKRTARRRYT | 73 |
| WP_002263887 | 1 | MKKP-YSIGLDIGTNSVGWAVVTDDYKVPAKKMKVLGNTDKSHIEKNLLGALLFDSGNTA--EDRRLKRTARRRYT | 73 |
| WP_002264920 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002269043 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002269448 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002271977 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002272766 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002273241 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002275430 | 1 | MKKP-YSIGLDIGTNSVGWAVVTDDYKVPAKKMKVLGNTDKSHIKKNLLGALLFDSGNTA--ADRRLKRTARRRYT | 73 |
| WP_002276448 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002277050 | 1 | MKKS-YSIGLDIGTNSVGWAVVTDDYKVPAKKMKVLGNTDKSHIKKNLLGALLFDSGNTA--EDRRLKRTARRRYT | 73 |
| WP_002277364 | 1 | MKKP-YSIGLDIGTNSVGWAVVTDDYKVPAKKMKVLGNTDKSHIKKNLLGALLFDSGNTA--ADRRLKRTARRRYT | 73 |
| WP_002279025 | 1 | MKKP-YSIGLDIGTNSVGWAVVTDDYKVPAKKMKVLGNTDKSHIEKNLLGALLFDSGNTA--EDRRLKRTARRRYT | 73 |
| WP_002279859 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002280230 | 1 | MKKP-YSIGLDIGTNSVGWAVVTDDYKVPAKKMKVLGNTDKSHIKKNLLGALLFDSGNTA--ADRRLKRTARRRYT | 73 |
| WP_002281696 | 1 | MKKP-YSIGLDIGTNSVGWAVVTDDYKVPAKKMKVLGNTDKSHIKKNLLGALLFDSGNTA--ADRRLKRTARRRYT | 73 |
| WP_002282247 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002282906 | 1 | MKKP-YSIGLDIGTNSVGWSVVTDDYKVPAKKMKVLGNTDKSHIEKNLLGALLFDSGNTA--EDRRLKRTARRRYT | 73 |
| WP_002283846 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002287255 | 1 | MKKP-YSIGLDIGTNSVGWAVVTDDYKVSAKKMKVLGNTDKSHIKKNLLGALLFDSGNTA--EDRRLKRTARRRYT | 73 |
| WP_002288990 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002289641 | 1 | MKKP-YSIGLDIGTNSVGWAVVTDDYKVPAKKMKVLGNTDKSHIKKNLLGALLFDSGNTA--EDRRLKRTTRRRYT | 73 |
| WP_002290427 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002295753 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002296423 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002304487 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002305844 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002307203 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002310390 | 1 | MKKP-YSIGLDIGTNSVGWAVVTDDYKVPAKKMKVLGNTDKSHIKKNLLGALLFDSGNTA--EDRRLKRTARRRYT | 73 |
| WP_002352408 | 1 | MKKP-YSIGLDIGTNSVGWAVVTDDYKVPAKKMKVLGNTDKSHIKKNLLGALLFDSGNTA--EDRRLKRTARRRYT | 73 |
| WP_012997688 | 1 | MKKP-YSIGLDIGTNSVGWAVVTDDYKVPAKKMKVLGNTDKSHIKKNLLGALLFDSGNTA--ADRRLKRTARRRYT | 73 |
| WP_014677909 | 1 | MKKP-YSIGLDIGTNSVGWAVVTDDYKVPDKKMKVLGNTDKSHIKKNLLGALLFDSGNTA--EDRRLKRTARRRYT | 73 |
| WP_019312892 | 1 | MKKP-YSIGLDIGTNSVGWAVVTDDYKVPAKKMKVLGNTDKSHIKKNLLGALLFDSGNTA--ADRRLKRTARRRYT | 73 |
| WP_019313659 | 1 | MKKP-YSIGLDIGTNSVGWAVVTDDYKVPAKKMKVLGNTDKSHIEKNLLGALLFDSGNTA--EDRRLKRTARRRYT | 73 |
| WP_019314093 | 1 | MKKP-YSIGLDIGTNSVGWAVVTDDYKVPAKKMKVLGNTDKSHIKKNLLGALLFDSGNTA--EDRRLKRTARRRYT | 73 |
| WP_019315370 | 1 | MKKP-YSIGLDIGTNSVGWAVVTDDYKVPAKKMKVLGNTDKSHIKKNLLGALLFDSGNTA--EDRRLKRTARRRYT | 73 |
| WP_019803776 | 1 | MKKP-YSIGLDIGTNSVGWAVVTDDYKVPAKKMKVLGNTDKSHIEKNLLGALLFDSGNTA--EDRRLKRTARRRYT | 73 |
| WP_019805234 | 1 | MKKP-YSIGLDIGTNSVGWAVVTDDYKVPAKKMKVLGNTDKSHIKKNLLGALLFDSGNTA--EDRRLKRTARRRYT | 73 |
| WP_024783594 | 1 | MKKP-YSIGLDIGTNSVGWAVVTDDYKVPAKKMKVLGNTDKSHIEKNLLGALLFDSGNTA--EDRRLKRTARRRYT | 73 |
| WP_024784288 | 1 | MKKP-YSIGLDIGTNSVGWAVVTDDYKVPAKKMKVLGNTDKSHIKKNLLGALLFDSGNTA--EDRRLKRTARRRYT | 73 |
| WP_024784666 | 1 | MKKP-YSIGLDIGTNSVGWAVVTDDYKVPAKKMKVLGNTDKSHIKKNLLGALLFDSGNTA--ADRRLKRTARRRYT | 73 |
| WP_024784894 | 1 | MKKP-YSIGLDIGTNSVGWAVVTDDYKVPAKKMKVLGNTDKSHIKKNLLGALLFDSGNTA--EDRRLKRTTRRRYT | 73 |
| WP_024786433 | 1 | MKKP-YSIGLDIGTNSVGWAVVTDDYKVPAKKMKVLGNTDKSHIKKNLLGALLFDSGNTA--ADRRLKRTARRRYT | 73 |
| WP_049473442 | 1 | MKKP-YSIGLDIGTNSVGWAVVTDDYKVPAKKMKVLGNTDKSHIKKNLLGALLFDSGNTA--ADRRLKRTARRRYT | 73 |
| WP_049474547 | 1 | MKKP-YSIGLDIGTNSVGWAVVTDDYKVPAKKMKVLGNTDKSHIKKNLLGALLFDSGNTA--EDRRLKRTTRRRYT | 73 |
| EMC03581 | 1 | MDL--------IGTNSVGWAVVIDDYKVPAKKMKVLGNTDKSHIKKNLLGALLFDSGNTA--ADRRLKRTARRRYT | 66 |
| WP_000428612 | 1 | ENKN-YSIGLDIGTNSVGWAVITDDYKVPSKKMKVLGNTDKRFIKKNLIGALLFDEGTTA--EARRLKRTARRRYT | 74 |
| WP_000428613 | 1 | ENKN-YSIGLDIGTNSVGWSVITDDYKVPSKKMKVLGNTDKRFIKKNLIGALLFDEGTTA--EARRLKRTARRRYT | 74 |
| WP_049523028 | 1 | K-KP-YSIGLDIGTNSVGWAVITDDYKVPAKKMKVLGNINKESIKKNLIGALLFDAGNTA--ADRRLKRTARRRYT | 73 |
| WP_003107102 | 1 | --------------------------------MKVLGNTDRQTVKKNMIGTLLFDSGETA--EARRLKRTARRRYT | 42 |
| WP_054279288 | 1 | -KKS-YSIGLDIGTNSVGWAVITDDYKVPAKKMKVLGNTSRQSIKKNMIGALLFDEGGPA--ASTRVKRTTRRRYT | 75 |
| WP_049531101 | 1 | SNKP-YSIGLDIGTNSVGWAVITDDYKVPSKKMKVLGNTDKHFIKKNLIGALLFDEGTTA--EDRRLKRTARRRYT | 74 |
| WP_049538452 | 1 | SNKP-YSIGLDIGTNSVGWVIITDDYKVPSKKMKVLGNTDKHFIKKNLIGALLFDEGTTA--EDRRLKRTARRRYT | 74 |
| WP_049549711 | 1 | SNKP-YSIGLDIGTNSVGWAVITDDYKVPSKKMTVLGNTDKHFIKKNLIGALLFDEGTTA--EDRRLKRTARRRYT | 74 |
| WP_007896501 | 1 | --YS-YSIGLDIGTNSVGWAVINEDYKVPAKKMTVFGNTDRKTIKKNLLGTVLFDSGETA--QARRLKRINRRRYT | 75 |
| EFR44625 | 1 | -----------------------------------------------MLGTVLFDSGETA--QARRLKRTNRRRYT | 27 |
| WP_002897477 | 1 | K-KP-YSIGLDIGTNSVGWAVVTDDYKVPAKKMRVFGDTDRSHIKKNLLGTLLFDDGNTA--ESRRLKRTARRRYT | 73 |
| WP_002906454 | 1 | K-KP-YSIGLDIGTNSVGWAVITDDYKVPSKKMKVLGNTDKHFIKKNLIGALLFDEGTTA--EDRRLKRTSRRRYT | 73 |
| WP_009729476 | 1 | ENKN-YSIGLDIGTNSVGWSVITDDYKVPSKKMKVLGNTDKHFIKKNLIGALLFDEGTTA--EARRLKRTARRRYT | 74 |
| CQR24647 | 1 | MKKP-YSIGLDIGTNSVGWSVVTDDYKVPAKKMKVLGNTDKEYIKKNLIGALLFDSGETA--EATRMKRTARRRYT | 73 |
| WP_000066813 | 1 | SNKS-YSIGLDIGTNSVGWAVITDDYKVPSKKMKVLGNTDKHFIKKNLIGALLFDEGTTA--EDRRLKRTARRRYT | 74 |
| WP_009754323 | 1 | NNNN-YSIGLDIGTNSVGWAVITDDYKVPSKKMRVLGNTDKRFIKKNLIGALLFDEGTTA--EDRRLKRTARRRYT | 74 |
| WP_044674937 | 1 | MKKK-YAIGIDIGTNSVGWSVVIDDYKVPSKKMKVFGNTEKRYIKKNLLGTLLFDEGNTA--ENRRLKRTARRRYT | 73 |
| WP_044676715 | 1 | MKKK-YAIGIDIGTNSVGWSVVTDDYKVPSKKMKVFGNTEKRYIKKNLLGTLLFDEGNTA--ENRRLKRTARRRYT | 73 |
| WP_044680361 | 1 | MKKK-YAIGIDIGTNSVGWSVVTDDYKVPSKKMKVFGNTEKRYIKKNLLGTLLFDEGNTA--ENRRLKRTARRRYT | 73 |
| WP_044681799 | 1 | MKKK-YAIGIDIGTNSVGWSVVTDDYKVPSKKMKVFGNTEKRYIKKNLLGTLLFDEGNTA--ENRRLKRTARRRYT | 73 |
| WP_049533112 | 1 | MDQK-YSIGLDIGTNSVGWAVVTDDYKVPAKKMKVLGNTDKQSIKKNLLGALLFDSGETA--EATRLKRTARRRYT | 73 |
| WP_029090905 | 1 | -----------------------------------------------MWGVSLFEAGKTA--AERRGYRSTRRRLN | 27 |
| WP_006506696 | 1 | I-VD-YCIGLDLGTGSVGWAVVDMNHRLMKRN------------GKHLWGSRLFSNAETA--ANRRASRSIRRRYN | 60 |
| AIT42264 | 1 | MDKK-YSIGLDIGTNSVGWAVITDEYKVPSKKFKVLGNTDRHSIKKNLIGALLFDSGETA--EATRLKRTARRRYT | 73 |
| WP_034440723 | 1 | -MKN-YTIGLDIGTNSVGWAVIKDDLTLVRKKIKISGNTDKKEVKKNLWGSFLFEQGDTA--QDTRVKRIARRRYE | 72 |
| AKQ21048 | 1 | MDKK-YSIGLDIGTNSVGWAVITDEYKVPSKKFKVLGNTDRHSIKKNLIGALLFDSGETA--EATRLKRTARRRYT | 73 |
| WP_004636532 | 1 | MQKN-YTIGLDIGTNSVGWAVMKDDYTLIRKRMKVLGNTDIKKIKKNFWGVRLFDEGETA--KETRLKRGTRRRYQ | 73 |
| WP_002364836 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_016631044 | 1 | --------------------------------------------------MRLFEEGHTA--EDRRLKRTARRRIS | 24 |
| EMS75795 | | ---------------------------------------------------------------------------- | |
| WP_002373311 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_002378009 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_002407324 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_002413717 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_010775580 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_010818269 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_010824395 | 1 | MKKD-YVIGLDIGSNSVGWAVMTEDYQLVKKKMPIYGNTEKKKIKKNFWGVRLFEEGHTA--EDRRLKRTARRRIS | 73 |
| WP_016622645 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_033624816 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_033625576 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_033789179 | 1 | MKKD-YVIGLDIGTNSVGWAVMTEDYQLVKKKMPIYGNTEKKKIKKNFWGVRLFEEGHTA--EDRRLKRTARRRIS | 73 |
| WP_002310644 | 1 | MKKE-YTIGLDIGTNSVGWSVLTDDYRLVSKKMKVAGNTEKSSTKKNFWGVRLFDEGQTA--EARRSKRTARRRLA | 73 |
| WP_002312694 | 1 | MKKE-YTIGLDIGTNSVGWSVLTDDYRLVSKKMKVAGNTEKSSTKKNFWGVRLFDEGQTA--EARRSKRTARRRLA | 73 |
| WP_002314015 | 1 | MKKE-YTIGLDIGTNSVGWSVLTDDYRLVSKKMKVAGNTEKSSTKKNFWGVRLFDEGQTA--EARRSKRTARRRLA | 73 |
| WP_002320716 | 1 | MKKE-YTIGLDIGTNSVGWSVLTDDYRLVSKKMKVAGNTEKSSTKKNFWGVRLFDEGQTA--EARRSKRTARRRLA | 73 |
| WP_002330729 | 1 | MKKE-YTIGLDIGTNSVGWSVLTDDYRLVSKKMKVAGNTEKSSTKKNFWGVRLFDEGQTA--EARRSKRTARRRLA | 73 |
| WP_002335161 | 1 | MKKE-YTIGLDIGTNSVGWSVLTDDYRLVSKKMKVAGNTEKSSTKKNFWGVRLFDEGQTA--EARRSKRTARRRLA | 73 |
| WP_002345439 | 1 | MKKE-YTIGLDIGTNSVGWSVLTDDYRLVSKKMKVAGNTEKSSTKKNFWGVRLFDEGQTA--EARRSKRTARRRLA | 73 |
| WP_034867970 | 1 | MTKD-YTIGLDIGTNSVGWAVLTDDYQLMKRKMSVHGNTEKKKIKKNEWGARLFDEGQTA--EFRRTKRINRRRLA | 73 |
| WP_047937432 | 1 | MKKE-YTIGLDIGTNSVGWSVLTDDYRLVSKKMKVAGNTEKSSTKKNFWGVRLFDEGQTA--EARRSKRTARRRLA | 73 |
| WP_010720994 | 1 | MTKD-YTIGLDIGTNSVGWAVLTDDYQLMKRKMSVHGNTEKKKIKKNFWGARLFDEGQTA--EFRRTKRINRRRLA | 73 |
| WP_010737004 | 1 | MTKD-YTIGLDIGTNSVGWAVLTDDYQLMKRKMSVHGNTEKKKIKKNFWGARLFDEGQTA--EFRRTKRINRRRLA | 73 |
| WP_034700478 | 1 | MTKD-YTIGLDIGTNSVGWAVLTDDYQLMKRKMSVHGNTEKKKIKKNFWGARLFDEGQTA--EFRRTKRINRRRLA | 73 |
| WP_007209003 | 1 | MKND-YTIGLDIGTNSVGYSVVTDDYKVISKKMNVFGNTEKKSIKKNFWGVRLFESGOTA--QEARMKRTSRRRIA | 73 |
| WP_023519017 | 1 | MEKE-YTIGLDIGTNSVGWAVLTDDYRLVARKMSIQGDSNRKKIKKNFWGARLFEEGKTA--QFRRIKRTNRRRIA | 73 |
| WP_010770040 | 1 | MKKE-YTIGLDIGTNSVGWAVLTENYDLVKKKMKVYGNTETKYLKKNLWGVRLFDEGETA--ADRRLKRTTRRRYS | 73 |
| WP_048604708 | 1 | MGKE-YTIGLDIGTNSVGWAVLQEDLDLVRRKMKVYGNTEKNYLKKNFWGVDLFDEGMTA--KDTRLKRTTRRRYF | 73 |
| WP_010750235 | 1 | MNKA-YTLGLDIGTNSVGWAVVTDDYRLMAKKMPVHSKMEKKKIKKNEWGARLFDEGQTA--EERRNKRATRRRLR | 73 |
| AII16583 | 1 | ADKK-YSIGLDIGTNSVGWAVITDEYKVPSKKFKVLGNTDRHSIKKNLIGALLFDSGETA--EATRLKRTARRRYT | 112 |
| WP_029073316 | 1 | NNKI-YNIGLDIGDASVGWAVVDEHYNLLKRH------------GKHMWGSRLFTQANTA--VERRSSRSTRRRYN | 65 |
| WP_031589969 | 1 | NNKI-YNIGLDIGDASVGWAVVDEHYNLLKRH------------GKHMWGSRLFTQANTA--VERRSSRSTRRRYN | 65 |
| KDA45870 | 1 | LKKD-YSIGLDIGTNSVGHAVVIDDYKVPTKKMKVFGDISKKTIKKNMLGVLLFNEGQTA--ADTRLKRGARRRYT | 74 |
| WP_039099354 | 1 | MSRP-YNIGLDIGTSSIGWSVVDDQSKLVSVR------------GKYGYGVRLYDEGQTA--AERRSFRTTRRRLK | 61 |
| AKP02966 | 1 | KEQP-YNIGLDIGTGSVGWAVINDNYDLLNIK------------KKNLWGVRLFEGAQTA--KETRLNRSTRRRYR | 64 |
| WP_010991369 | 1 | MKKP-YTIGLDIGTNSVGWAVLTDQYDLVKRKMKIAGDSEKKQIKKNFWGVRLFDEGQTA--ADRRMARTARRRIE | 73 |
| WP_033838504 | 1 | MKKP-YTIGLDIGTNSVGWAVLTDQYDLVKRKMKIAGDSEKKQIKKNFWGVRLFDEGQTA--ADRRMARTARRRIE | 73 |
| EHN60060 | 1 | MKKP-YTIGLDIGTNSVGWAVLTDQYDLVKRKMKIAGDSEKKQIKKNFWGVRLFDEGQTA--ADRRMARTARRRIE | 76 |
| EFR89594 | | ---------------------------------------------------------------------------- | |
| WP_038409211 | 1 | MRKP-YTIGLDIGTNSVGWAVLTDQYNLVKRKMKVAGSAEKKQIKKNFWGVRLFDEGEVA--AGRRMNRTTRRRIE | 73 |
| EFR95520 | | ---------------------------------------------------------------------------- | |
| WP_003723650 | 1 | MKNP-YTIGLDIGTNSVGWAVLINQYDLVKRKMKVAGNSDKKQIKKNFWGVRLFDDGQTA--VDRRMNRTARRRIE | 73 |
| WP_003727705 | 1 | MKNP-YTIGLDIGTNSVGWAVLTDQYDLVKRKMKVAGNSDKKQIKKNFWGVRLFDDGQTA--VDRRMNRTARRRIE | 73 |
| WP_003730785 | 1 | MKNP-YTIGLDIGTNSVGWAVLTDQYDLVKRKMKVAGNSDKKQIKKNFWGVRLFDDGQTA--VDRRMNRTARRRIE | 73 |
| WP_003733029 | 1 | MKKP-YTIGLDIGTNSVGWAVLTDQYDLVKRKMKISGDSEKKQIKKNFWGVRLFEKGETA--AKRRMSRTARRRIE | 73 |
| WP_003739838 | 1 | MKNP-YTIGLDIGTNSVGWAVLTDQYDLVKRKMKVAGNSDKKQIKKNFWGVRLFDEGETA--ADRRMNRTARRRIE | 73 |
| WP_014601172 | 1 | MKNP-YTIGLDIGTNSVGWAVLINQYDLVKRKMKVAGNSDKKQIKKNFWGVRLFDDGQTA--VDRRMNRTARRRIE | 73 |
| WP_023548323 | 1 | MKNP-YTIGLDIGTNSVGWAVLINQYDLVKRKMKVAGNSDKKQIKKNFWGVRLFDDGQTA--VDRRMNRTARRRIE | 73 |
| WP_031665337 | 1 | MKNP-YTIGLDIGTNSVGWAVLTDQYDLVKRKMKVAGNSDKKQIKKNFWGVRLFDDGQTA--VDRRMNRTARRRIE | 73 |
| WP_031669209 | 1 | MKKP-YTIGLDIGTNSVGWAVLTDQYDLVKRKMKISGDSEKKQIKKNFWGVRLFEKGETA--AKRRMSRTARRRIE | 73 |
| WP_033920898 | 1 | MKNP-YTIGLDIGTNSVGWAVLTNQYDLVKRKMKVAGNSDKKQIKKNFWGVRLFDDGQTA--VDRRMNRTARRRIE | 73 |
| AKI42028 | 1 | MKNP-YTIGLDIGTNSVGWAVLTNQYDLVKRKMKVAGNSDKKQIKKNFWGVRLFDDGQTA--VDRRMNRTARRRIE | 76 |
| AKI50529 | 1 | MKNP-YTIGLDIGTNSVGWAVLTNQYDLVKRKMKVAGNSDKKQIKKNFWGVRLFDDGQTA--VDRRMNRTARRRIE | 76 |
| EFR83390 | | ---------------------------------------------------------------------------- | |
| WP_046323366 | 1 | MKKP-YTIGLDIGTNSVGWAALTDQYDLVKRKMKVAGNSEKKQIKKNLWGVRLVDEGKTA--AHRRVNRTTRRRIE | 73 |
| AKE81011 | 1 | ADKK-YSIGLDIGTNSVGWAVITDEYKVPSKKFKVLGNTDRHSIKKNLIGALLFDSGETA--EATRLKRTARRRYT | 89 |
| CUO82355 | 1 | I-VD-YCIGLDLGTGSVGWAVVDMNHRLMKRN------------GKHLWGSRLFSNAETA--ATRRSSRSIRRRYN | 64 |
| WP_033162887 | 1 | KDIR-YSIGLDIGTNSVGWAVMDEHYELLKKG------------NHHMWGSRLFDAAEPA--ATRRASRSIRRRYN | 65 |
| AGZ01981 | 1 | ADKK-YSIGLDIGTNSVGWAVITDEYKVPSKKFKVLGNTDRHSIKKNLIGALLFDSGETA--EATRLKRTARRRYT | 106 |
| AKA60242 | 1 | MDKK-YSIGLAIGTNSVGWAVITDEYKVPSKKFKVLGNTDRHSIKKNLIGALLFDSGETA--EATRLKRTARRRYT | 73 |
| AKS40380 | 1 | MDKK-YSIGLDIGTNSVGWAVITDEYKVPSKKFKVLGNTDRHSIKKNLIGALLFDSGETA--EATRLKRTARRRYT | 73 |
| 4UN5_B | 1 | MDKK-YSIGLAIGTNSVGWAVITDEYKVPSKKFKVLGNTDRHSIKKNLIGALLFDSGETA--EATRLKRTARRRYT | 77 |
| WP_010922251 | 74 | 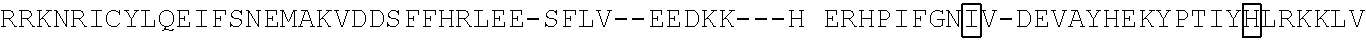 | 143 |
| WP_039695303 | 75 | RRKNRLRYLQEIFANEIAKVDESFFQRLDE-SFLT--DDDKT---F DSHPIFGNKA-EEDAYHQKFPTIYHLRKHLA | 144 |
| WP_045635197 | 74 | RRKNRLRYLQEIFSEEMSKVDSSFFHRLDD-SFLI--PEDKR---E SKYPIFATLT-EEKEYHKQFPTIYHLRKQLA | 143 |
| 5AXW_A | 62 | RRRHRIQRVKKLLFD---------YNLLTDhSELS----------G --NPYEARVK--------------GLSQKLS | 104 |
| WP_009880683 | | ---------------------------------------------- ------------------------------- | |
| WP_010922251 | 74 | RRKNRICYLQEIFSNEMAKVDDSFFHRLEE-SFLV--EEDKK---H ERHPIFGNIV-DEVAYHEKYPTIYHLRKKLV | 143 |
| WP_011054416 | 74 | RRKNRICYLQEIFSNEMAKVDDSFFHRLEE-SFLV--EEDKK---H ERHPIFGNIV-DEVAYHEKYPTIYHLRKKLA | 143 |
| WP_011284745 | 74 | RRKNRICYLQEIFSNEMAKVDDSFFHRLEE-SFLV--EEDKK---H ERHPIFGNIV-DEVAYHEKYPTIYHLRKKLV | 143 |
| WP_011285506 | 74 | RRKNRICYLQEIFSNEMAKVDDSFFHRLEE-SFLV--EEDKK---H ERHPIFGNIV-DEVAYHEKYPTIYHLRKKLV | 143 |
| WP_011527619 | 74 | RRKNRICYLQEIFSNEMAKVDDSFFHRLEE-SFLV--EEDKK---H ERHPIFGNIV-DEVAYHEKYPTIYHLRKKLV | 143 |
| WP_012560673 | 74 | RRKNRICYLQEIFSNEIAKVDDSFFHRLEE-SFLV--EEDKK---H ERHPIFGNIV-DEVAYHEKYPTIYHLRKKLA | 143 |
| WP_014407541 | 74 | RRKNRICYLQEIFSNEMAKVDDSFFHRLEE-SFLV--EEDKK---H ERHPIFGNIV-DEVAYHEKYPTIYHLRKKLA | 143 |
| WP_020905136 | 74 | RRKNRICYLQEIFSNEMAKVDDSFFHRLEE-SFLV--EEDKK---H ERHPIFGNIV-DEVAYHEKYPTIYHLRKKLV | 143 |
| WP_023080005 | 74 | RRKNRICYLQEIFSNEMAKVDDSFFHRLEE-SFLV--EEDKK---H ERHPIFGNIV-DEVAYHEKYPTIYHLRKKLA | 143 |
| WP_023610282 | 74 | RRKNRICYLQEIFSNEMAKVDDSFFHRLEE-SFLV--EEDKK---H ERHPIFGNIV-DEVAYHEKYPTIYHLRKKLA | 143 |
| WP_030125963 | 74 | RRKNRICYLQEIFSNEMAKVDDSFFHRLEE-SFLV--EEDKK---H ERHPIFGNIV-DEVAYHEKYPTIYHLRKKLA | 143 |
| WP_030126706 | 74 | RRKNRICYLQEIFSNEMAKVDDSFFHRLEE-SFLV--EEDKK---H ERHPIFGNIV-DEVAYHEKYPTIYHLRKKLA | 143 |
| WP_031488318 | 74 | RRKNRICYLQEIFSNEMAKVDDSFFHRLEE-SFLV--EEDKK---H ERHPIFGNIV-DEVAYHEKYPTIYHLRKKLA | 143 |
| WP_032460140 | 74 | RRKNRICYLQEIFSNEIAKVDDSFFHRLEE-SFLV--EEDKK---H ERHPIFGNIV-DEVAYHEKYPTIYHLRKKLA | 143 |
| WP_032461047 | 74 | RRKNRICYLQEIFSNEIAKVDDSFFHRLEE-SFLV--EEDKK---H ERHPIFGNIV-DEVAYHEKYPTIYHLRKKLA | 143 |
| WP_032462016 | 74 | RRKNRICYLQEIFSNEMAKVDDSFFHRLEE-SFLV--EEDKK---H ERHPIFGNIV-DEVAYHEKYPTIYHLRKKLV | 143 |
| WP_032462936 | 74 | RRKNRICYLQEIFSNEMAKVDDSFFHRLEE-SFLV--EEDKK---H ERHPIFGNIV-DEVAYHEKYPTIYHLRKKLA | 143 |
| WP_032464890 | 74 | RRKNRICYLQEIFSNEMAKVDDSFFHRLEE-SFLV--EEDKK---H ERHPIFGNIV-DEVAYHEKYPTIYHLRKKLV | 143 |
| WP_033888930 | | ---------------------------------------------- ------------------------------- | |
| WP_038431314 | 74 | RRKNRICYLQEIFSNEMAKVDDSFFHRLEE-SFLV--EEDKK---H ERHPIFGNIV-DEVAYHEKYPTIYHLRKKLA | 143 |
| WP_038432938 | 74 | RRKNRICYLQEIFSNEMAKVDDSFFHRLEE-SFLV--EEDKK---H ERHPIFGNIV-DEVAYHEKYPTIYHLRKKLA | 143 |
| WP_038434062 | 74 | RRKNRICYLQEIFSNEMAKVDDSFFHRLEE-SFLV--EEDKK---H ERHPIFGNIV-DEVAYHEKYPTIYHLRKKLA | 143 |
| BAQ51233 | 1 | ----------------MAKVDDSFFHRLEE-SFLV--EEDKK---H ERHPIFGNIV-DEVAYHEKYPTIYHLRKKLV | 54 |
| KGE60162 | | ---------------------------------------------- ------------------------------- | |
| KGE60856 | | ---------------------------------------------- ------------------------------- | |
| WP_002989955 | 74 | RRKNRICYLQEIFSNEMAKVDDSFFHRLEE-SFLV--EEDKK---H ERHPIFGNIV-DEVAYHEKYPTIYHLRKKLV | 143 |
| WP_003030002 | 74 | RRRNRLRYLQEIFAEEMNKVDENFFQRLDD-SELV--DEDKR---G ERHPIFGNIA-AEVKYHDDFPTIYHLRKHLA | 143 |
| WP_003065552 | 75 | RRKNRLRYLQEIFAEEMTKVDESFFQRLDE-SFLRwdDDNKK---L GRYPIFGNIA-DVVKYHQEFPTIYHLRKHLA | 146 |
| WP_001040076 | 74 | RRRNRILYLQEIFAEEMSKVDDSFFHRLED-SFLV--EEDKR---G SKYPIFATLQ-EEKYYHEKFPTIYHLRKELA | 143 |
| WP_001040078 | 74 | RRRNRILYLQEIFAEEMSKVDDSFFHRLED-SFLV--EEDKR---G SKYPIFATLQ-EEKDYHEKFPTIYHLRKELA | |
| WP_001040080 | 74 | RRRNRILYLQEIFAEEMSKVDDSFFHRLED-SFLV--EEDKR---G SKYPIFATLQ-EEKDYHEKFSTIYHLRKELA | 143 |
| WP_001040081 | 74 | RRRNRILYLQEIFAEEMSKVDDSFFHRLED-SFLV--EEDKR---G SKYPIFATLQ-EEKDYHEKFSTIYHLRKELA | 143 |
| WP_001040083 | 74 | RRRNRILYLQEIFAEEMSKVDDSFFHRLED-SFLV--EEDKR---G SKYPIFATLQ-EEKDYHEKFSTIYHLRKELA | 143 |
| WP_001040085 | 74 | RRRNRILYLQEIFAEEMSKVDDSFFHRLED-SFLV--EEDKR---G SKYPIFATLQ-EEKDYHEKFSTIYHLRKELA | 143 |
| WP_001040087 | 74 | RRRNRILYLQEIFAEEMSKVDDSFFHRLED-SFLV--EEDKR---G SKYPIFATLQ-EEKDYHEKFSTIYHLRKELA | 143 |
| WP_001040088 | 74 | RRRNRILYLQEIFAEEMSKVDDSFFHRLED-SFLV--EEDKR---G SKYPIFATLQ-EEKDYHEKFSTIYHLRKELA | 143 |
| WP_001040089 | 74 | RRRNRILYLQEIFAEEMSKVDDSFFHRLED-SELV--EEDKR---G SKYPIFATLQ-EEKDYHEKFSTIYHLRKELA | 143 |
| WP_001040090 | 74 | RRRNRILYLQEIFAEEMSKVDDSFFHRLED-SFLV--EEDKR---G SKYPIFATLQ-EEKDYHEKFSTIYHLRKELA | 143 |
| WP_001040091 | 74 | RRRNRILYLQEIFAEEMSKVDDSFFHRLED-SFLV--EEDKR---G SKYPIFATLQ-EEKDYHEKFSTIYHLRKELA | 143 |
| WP_001040092 | 74 | RRRNRILYLQEIFAEEMSKVDDSFFHRLED-SELV--EEDKR---G SKYPIFATMQ-EEKDYHEKFPTIYHLRKELA | 143 |
| WP_001040094 | 74 | RRRNRILYLQEIFAEEMSKVDDSFFHRLED-SFLV--EEDKR---G SKYPIFATMQ-EEKYYHEKFPTIYHLRKELA | 143 |
| WP_001040095 | 74 | RRRNRILYLQEIFAEEMSKVDDSFFHRLED-SELV--EEDKR---G SKYPIFATMQ-EEKYYHEKFPTIYHLRKELA | 143 |
| WP_001040096 | 74 | RRRNRILYLQEIFAEEMSKVDDSFFHRLED-SFLV--EEDKR---G SKYPIFATMQ-EEKYYHEKFPTIYHLRKELA | 143 |
| WP_001040097 | 74 | RRRNRILYLQEIFAEEMSKVDDSFFHRLED-SFLV--EEDKR---G SKYPIFATMQ-EEKYYHEKFPTIYHLRKELA | 143 |
| WP_001040098 | 74 | RRRNRILYLQEIFAEEMSKVDDSFFHRLED-SFLV--EEDKR---G SKYPIFATMQ-EEKYYHEKFPTIYHLRKELA | 143 |
| WP_001040099 | 74 | RRRNRILYLQEIFAEEMSKVDDSFFHRLED-SFLV--EEDKR---G SKYPIFATMQ-EEKYYHEKFPTIYHLRKELA | 143 |
| WP_001040100 | 74 | RRRNRILYLQEIFAEEMSKVDDSFFHRLED-SFLV--EEDKR---G SKYPIFATMQ-EEKYYHEKFPTIYHLRKELA | 143 |
| WP_001040104 | 74 | RRRNRILYLQEIFAEEMSKVDDSFFHRLED-SFLV--EEDKR---G SKYXIFATLQ-EEKDYHEKFSTIYHLRKELA | 143 |
| WP_001040105 | 74 | RRRNRILYLQEIFAEEMSKVDDSFFHRLED-SELV--EEDKR---G SKYPIFATLQ-EEKDYHEKFSTIYHLRKELA | 143 |
| WP_001040106 | 74 | CRRNRILYLQEIFAEEMSKVDDSFFHRLED-SFLV--EDDKR---G SKYPIFATMQ-EEKDYHEKFPTIYHLRKELA | 143 |
| WP_001040107 | 74 | RRRNRILYLQEIFAEEMSKVDDSFFHRLED-SFLV--EDDKR---G SKYPIFATMQ-EEKDYHEKFPTIYHLRKELA | 143 |
| WP_001040108 | 74 | RRRNRILYLQEIFAEEMSKVDDSFFHRLED-SFLV--EDDKR---G SKYPIFATMQ-EEKDYHEKFPTIYHLRKELA | 143 |
| WP_001040109 | 74 | RRRNRILYLQEIFAEEMSKVDDSFFHRLED-SELV--EDDKR---G SKYPIFATMQ-EEKDYHEKFPTIYHLRKELA | 143 |
| WP_001040110 | 74 | RRRNRILYLQEIFAEEMSKVDDSFFHRLED-SELV--EDDKR---G SKYPIFATMQ-EEKDYHEKFPTIYHLRKELA | 143 |
| WP_015058523 | 74 | RRRNRILYLQEIFAEEMSKVDDSFFHRLED-SFLV--EEDKR---G SKYPIFATMQ-EEKDYHEKFPTIYHLRKELA | 143 |
| WP_017643650 | 74 | RRRNRILYLQEIFAEEMSKVDDSFFHRLED-SFLV--EEDKR---G SKYPIFATMQ-EEKDYHEKFPTIYHLRKELA | 143 |
| WP_017647151 | 74 | RRRNRILYLQEIFAEEMSKVDDSFFHRLED-SFLV--EDDKR---G SKYPIFATMQ-EEKDYHEKFPTIYHLRKELA | 143 |
| WP_017648376 | 74 | RRRNRILYLQEIFAEEMSKVDDSFFHRLED-SELV--EDDKR---G SKYPIFATMQ-EEKDYHEKFPTIYHLRKELA | 143 |
| WP_017649527 | 74 | RRRNRILYLQEIFAEEMSKVDDSFFHRLED-SELV--EEDKR---G SKYPIFATLQ-EEKDYHEKFPTIYHLRKELA | 143 |
| WP_017771611 | 74 | RRRNRILYLQEIFAEEMSKVDDSFFHRLED-SELV--EDDKR---G SKYPIFATMQ-EEKDYHEKFPTIYHLRKELA | 143 |
| WP_017771984 | 74 | RRRNRILYLQEIFAEEMSKVDDSFFHRLED-SFLV--EEDKR---G SKYPIFATLQ-EEKDYHEKFSTIYHLRKELA | 143 |
| CFQ25032 | 74 | RRRNRILYLQEIFAEEMSKVDDSFFHRLED-SFLV--EEDKR---G SKYPIFATLQ-EEKDYHEKFSTIYHLRKELA | 143 |
| CFV16040 | 74 | RRRNRILYLQEIFAEEMSKVDDSFFHRLED-SFLV--EEDKR---G SKYPIFATLQ-EEKDYHEKFSTIYHLRKELA | 143 |
| KLJ37842 | 74 | RRRNRILYLQEIFAEEMSKVDDSFFHRLED-SFLV--EEDKR---G SKYPIFATLQ-EEKDYHEKFSTIYHLRKELA | 143 |
| KLJ72361 | 74 | RRRNRILYLQEIFAEEMSKVDDSFFHRLED-SFLV--EEDKR---G SKYPIFATLQ-EEKDYHEKFSTIYHLRKELA | 143 |
| KLL20707 | 74 | RRRNRILYLQEIFAEEMSKVDDSFFHRLED-SFLV--EEDKR---G SKYPIFATLQ-EEKDYHEKFSTIYHLRKELA | 143 |
| KLL42645 | 74 | RRRNRILYLQEIFAEEMSKVDDSFFHRLED-SFLV--EDDKR---G SKYPIFATMQ-EEKDYHEKFPTIYHLRKELA | 143 |
| WP_047207273 | 74 | RRRNRILYLQEIFAEEMSKVDDSFFHRLED-SELV--EEDKR---G SKYPIFATLQ-EEKDYHEKFSTIYHLRKELA | 143 |
| WP_047209694 | 74 | RRRNRILYLQEIFAEEMSKVDDSFFHRLED-SFLV--EEDKR---G SKYPIFATMQ-EEKYYHEKFPTIYHLRKELA | 143 |
| WP_050198062 | 74 | RRRNRILYLQEIFAEEMSKVDDSFFHRLED-SFLV--EEDKR---G SKYPIFATLQ-EEKDYHEKFSTIYHLRKELA | 143 |
| WP_050201642 | 74 | RRRNRILYLQEIFAEEMSKVDDSFFHRLED-SELV--EEDKR---G SKYPIFATLQ-EEKDYHEKFSTIYHLRKELA | 143 |
| WP_050204027 | 74 | RRRNRILYLQEIFAEEMSKVDDSFFHRLED-SFLV--EDDKR---G SKYPIFATMQ-EEKDYHEKFPTIYHLRKELA | 143 |
| WP_050881965 | 74 | RRRNRILYLQEIFAEEMSKVDDSFFHRLED-SFLV--EEDKR---G SKYPIFATlQ-EEKDYHEKFPTIYHLRKELA | 143 |
| WP_050886065 | 74 | RRRNRILYLQEIFAEEMSKVDDSFFHRLED-SFLV--EEDKR---G SKYPIFATLQ-EEKDYHEKFSTIYHLRKELA | 143 |
| AHN30376 | 74 | RRRNRILYLQEIFAEEMSKVDDSFFHRLED-SFLV--EEDKR---G SKYPIFATMQ-EEKDYHEKFPTIYHLRKELA | 143 |
| EAO78426 | 74 | RRRNRILYLQEIFAEEMSKVDDSFFHRLED-SELV--EEDKR---G SKYPIFATLQ-EEKDYHEKFSTIYHLRKELA | 143 |
| CCW42055 | 74 | RRRNRILYLQEIFAEKMSKVDDSFFHRLED-SFLV--EEDKR---G SKYPIFATLQ-EEKDYHEKFPTIYHLRKELA | 143 |
| WP_003041502 | 74 | RRRNRLRYLQEIFAEEMMQVDESFFQRLDD-SFLV--DEDKR---G ERHPIFGNIA-AEVKYHDEFPTIYHLRKHLA | 143 |
| WP_037593752 | 75 | RRKNRLRYLQEIFTEEMNKVDENFFQRLDD-SFLV--EEDKQ---G SKYPIFGTLK-EEKEYHKKFKTIYHLREELA | 144 |
| WP_049516684 | 75 | RRRNRLRYLQEIFAEEMMQVDESFFQRLDD-SELV--EEDKR---G SRYPIFGNIA-AEVKYHDDFPTIYHLRKHLV | 144 |
| GAD46167 | 74 | RRKNRLRYLQEIFTEEMNKVDENFFQRLDD-SFLV--EEDKQ---G SKYPIFATMQ-EEKDYHEKFPTIYHLRKELA | 143 |
| WP_018363470 | 75 | RRKNRLRYLQDIFTEEMAKVDDSFFQRLDE-SFLT--DNDKN---F DSHPIFGNKA-EEDAYHQKFPTIYHLRKHLA | 144 |
| WP_003043819 | 74 | RRKNRIRYLQEIFANEMAKLDDSFFQRLEE-SFLV--EEDKK---N ERHPIFGNLA-DEVAYHRNYPTIYHLRKKLA | 143 |
| WP_006269658 | 74 | RRKNRLRYLQEIFTGEMNKVDENFFORLDD-SFLV--DEDKR---G EHHPIFGNIA-AEVKYHDDFPTIYHLRRHLA | 143 |
| WP_048800889 | 74 | RRKNRLRYLQEIFIEEMNKVDENFFQRLDD-SFLV--TEDKR---G SKYPIFGTLK-EEKEYYKEFETIYHLRKRLA | 143 |
| WP_012767106 | 74 | RRKNRIRYLQEIFSSEMSKVDDSFFHRLEE-SFLV--EEDKK---H ERHPIFGNIV-DEVAYHEKYPTIYHLRKKLA | 143 |
| WP_014612333 | 74 | RRKNRIRYLQEIFSSEMSKVDDSFFHRLEE-SFLV--EEDKK---H ERHPIFGNIV-DEVAYHEKYPTIYHLRKKLA | 143 |
| WP_015017095 | 74 | RRKNRIRYLQEIFSSEMSKVDDSFFHRLEE-SFLV--EEDKK---H ERHPIFGNIV-DEVAYHEKYPTIYHLRKKLA | 143 |
| WP_015057649 | 74 | RRKNRIRYLQEIFSSEMSKVDDSFFHRLEE-SFLV--EEDKK---H ERHPIFGNIV-DEVAYHEKYPTIYHLRKKLA | 143 |
| WP_048327215 | 74 | RRKNRIRYLQEIFSSEMSKVDDSFFHRLEE-SFLV--EEDKK---H ERHPIFGNIV-DEVAYHEKYPTIYHLRKKLA | 143 |
| WP_049519324 | 74 | RRKNRIRYLQEIFSSEMSKVDDSFFHRLEE-SFLV--EEDKK---H ERHPIFGNIV-DEVAYHEKYPTIYHLRKKLA | 143 |
| WP_012515931 | 74 | RRKNRLRYLKEIFTEEMAKVDDGFFQRLED-SFYV--LEDKE---G NKHPIFANLA-DEVAYHEKYPTIYHLRKKLV | 143 |
| WP_021320964 | 74 | RRKNRLRYLKEIFTEEMAKVDDGFFQRLED-SFYV--LEDKE---G NKHPIFANLA-DEVAYHKKYPTIYHLRKELV | 143 |
| WP_037581760 | 74 | RRKNRLRFLKEIFTEEMAKVDDGFFQRLED-SFYV--LEDKE---G NKHPIFANLA-DEVAYHKKYPTIYHLRKELV | 143 |
| WP_004232481 | 74 | RRKNRLRYLQEIFAKEMAKVDESFFQRLEE-SFLT--DDDKT---F DSHPIFGNKA-EEDTYHQEFPTIYHLRKHLA | 143 |
| WP_009854540 | 75 | RRKNRLRYLQEIFAEEMTKVDESFFYRLDE-SFLT--TDEKD---F ERHPIFGNKA-EEDAYHQKFPTIYHLRNYLA | 144 |
| WP_012962174 | 75 | RRKNRLRYLQEIFAEEMAKVDESFFYRLDE-SFLT--TDDKD---F ERHPIFGNKA-DEIKYHQEFPTIYHLRKHLA | 144 |
| WP_039695303 | 75 | RRKNRLRYLQEIFANEIAKVDESFFQRLDE-SFLT--DDDKT---F DSHPIFGNKA-EEDAYHQKFPTIYHLRKHLA | 144 |
| WP_014334983 | 74 | RRKNRLRYLQEIFAKEMTKVDESFFQRLEE-SFLT--DDDKT---F DSHPIFGNKA-EEDAYHQKFPTIYHLRKYLA | 143 |
| WP_003099269 | 74 | RRKYRIKELQKIFSSEMNELDIAFFPRLSE-SFLV--SDDKE---F ENHPIFGNLK-DEITYHNDYPTIYHLRQTLA | 143 |
| AHY15608 | 74 | RRKYRIKELQKIFSSEMNELDIAFFPRLSE-SFLV--SDDKE---F ENHPIFGNLK-DEITYHNDYPTIYHLRQTLA | 143 |
| AHY17476 | 74 | RRKYRIKELQKIFSSEMNELDIAFFPRLSE-SELV--SDDKE---F ENHPIFGNLK-DEITYHNDYPTIYHLRQTLA | 143 |
| ESR09100 | | ---------------------------------------------- ------------------------------- | |
| AGM98575 | 74 | RRKYRIKELQKIFSSEMNELDIAFFPRLSE-SFLV--SDDKE---F ENHPIFGNLK-DEITYHNDYPTIYHLRQTLA | 143 |
| ALF27331 | 74 | RRRNRILYLQEIFSEEMGKVDDSFFHRLED-SELV--TEDKR---G ERHPIFGNLE-EEVKYHENFPTIYHLRQYLA | 143 |
| WP_018372492 | 74 | RRRNRIRYLQHIFAEEMNRADENFFHRLKE-SFFV--EEDKT---Y SKYPIFGTLE-EEKNYHKNYPTIYHLRKTLA | 143 |
| WP_045618028 | 75 | RRKNRLRYLQEIFTEEMSKVDISFFHRLDD-SFLV--PEDKR---G SKYPIFATLE-EEKEYHKNGPTIYHLRKHLA | 144 |
| WP_045635197 | 74 | RRKNRLRYLQEIFSEEMSKVDSSFFHRLDD-SFLI--PEDKR---E SKYPIFATLT-EEKEYHKQFPTIYHLRKQLA | 143 |
| WP_002263549 | 74 | RRRNRILYLQEIFSEEMGKVDDSFFHRLED-SFLV--TEDKR---G ERHPIFGNLE-EEVKYHENFPTIYHLRQYLA | 143 |
| WP_002263887 | 74 | RRRNRILYLQEIFSEEMGKVDDSFFHRLED-SFLV--TEDKR---G ERHPIFGNLE-EEVKYHENFPTIYHLRQYLA | 143 |
| WP_002264920 | 74 | RRRNRILYLQEIFSEEMGKVDDSFFHRLDE-SFLT--DDDKN---F DSYPIFGNKA-EEDAYHQKFPTIYHLRKHLA | 143 |
| WP_002269043 | 74 | RRRNRILYLQEIFSEEMGKVDDSFFHRLED-SFLV--TEDKR---G ERHPIFGNLE-EEVKYHENFPTIYHLRQYLA | 143 |
| WP_002269448 | 74 | RRRNRILYLQEIFSEEMGKVDDSFFHRLED-SFLV--TEDKR---G ERHPIFGNLE-EEVKYHENFPTIYHLRQYLA | 143 |
| WP_002271977 | 74 | RRRNRILYLQEIFSEEMGKVDDSFFHRLED-SFLV--TEDKR---G ERHPIFGNLE-EEVKYHENFPTIYHLRQYLA | 143 |
| WP_002272766 | 74 | RRRNRILYLQEIFSEEMGKVDDSFFHRLED-SFLV--TEDKR---G ERHPIFGNLE-EEVKYHENFPTIYHLRQYLA | 143 |
| WP_002273241 | 74 | RRRNRILYLQEIFSEEMGKVDDSFFHRLED-SFLV--TEDKR---G ERHPIFGNLE-EEVKYHENFPTIYHLRQYLA | 143 |
| WP_002275430 | 74 | RRRNRILYLQEIFAEEMSKVDDSFFHRLED-SFLV--TEDKR---G ERHPIFGNLE-EEVKYHENFPTIYHLRQYLA | 143 |
| WP_002276448 | 74 | RRRNRILYLQEIFSEEMGKVDDSFFHRLED-SFLV--TEDKR---G ERHPIFGNLE-EEVKYHENFPTIYHLRQYLA | 143 |
| WP_002277050 | 74 | RRRNRILYLQEIFSEEMGKVDDSFFHRLDE-SFLT--DDDKN---F DSHPIFGNKA-EEDAYHQKFPTIYHLRKHLA | 143 |
| WP_002277364 | 74 | RRRNRILYLQEIFAEEMSKVDDSFFHRLED-SFLV--TEDKR---G ERHPIFGNLE-EEVKYHENFPTIYHLRQYLA | 143 |
| WP_002279025 | 74 | RRRNRILYLQEIFSEEMGKVDDSFFHRLED-FFLV--TEDKR---G ERHPIFGNLE-EEVKYHENFPTIYHLRQYLA | 143 |
| WP_002279859 | 74 | RRRNRILYLQEIFSEEMGKVDDSFFHRLDE-SFLT--DDDKN---F DSHPIFGNKA-EEDAYHQKFPTIYHLRKHLA | 143 |
| WP_002280230 | 74 | RRRNRILYLQEIFAEEMSKVDDSFFHRLED-SFLV--TEDKR---G ERHPIFGNLE-EEVKYHENFPTIYHLRQYLA | 143 |
| WP_002281696 | 74 | RRRNRILYLQEIFAEEMSKVDDSFFHRLED-SFLV--TEDKR---G ERHPIFGNLE-EEVKYHENFPTIYHLRQYLA | 143 |
| WP_002282247 | 74 | RRRNRILYLQEIFAEEMSKVDDSFFHRLDE-SFLT--DDDKN---F DSHPIFGNKA-EEDAYHQKFPTIYHLRKHLA | 143 |
| WP_002282906 | 74 | RRRNRILYLQEIFSEEMGKVDDSFFHRLED-SFLV--TEDKR---G ERHPIFGNLE-EEVKYHENFPTIYHLRQYLA | 143 |
| WP_002283846 | 74 | RRRNRILYLQEIFSEEMGKVDDSFFHRLED-SELV--TEDKR---G ERHPIFGNLE-EEVKYHENFPTIYHLRQYLA | 143 |
| WP_002287255 | 74 | RRRNRILYLQEIFAEEMSKVDDSFFHRLED-SELV--TEDKR---G ERHPIFGNLE-EEVKYHENFPTIYHLRQYLA | 143 |
| WP_002288990 | 74 | RRRNRILYLQEIFSEEMGKVDDSFFHRLED-SELV--TEDKR---G ERHPIFGNLE-EEVKYHENFPTIYHLRQYLA | 143 |
| WP_002289641 | 74 | RRRNRILYLQEIFSEEMGKVDDSFFHRLED-SELV--TEDKR---G ERHPIFGNLE-EEVKYHENFPTIYHLRQYLA | 143 |
| WP_002290427 | 74 | RRRNRILYLQEIFSEEMGKVDDSFFHRLED-SFLV--TEDKR---G ERHPIFGNLE-EEVKYHENFPTIYHLRQYLA | 143 |
| WP_002295753 | 74 | RRRNRILYLQEIFSEEMGKVNDSFFHRLED-SELV--TEDKR---G ERHPIFGNLE-EEVKYHENFPTIYHLRQYLA | 143 |
| WP_002296423 | 74 | RRRNRILYLQEIFSEEMGKVDDSFFHRLED-SFLV--TEDKR---G ERHPIFGNLE-EEVKYHENFPTIYHLRQYLA | 143 |
| WP_002304487 | 74 | RRRNRILYLQEIFAEEMMQVDESFFQRLDD-SFLV--EEDKR---G SRYPIFGTLK-EEKKYHKEFKTIYHLREKLA | 143 |
| WP_002305844 | 74 | RRRNRILYLQEIFSEEMDKVDDSFFHRLED-SFLV--TEDKR---G ERHPIFGNLE-EEVKYHENFPTIYHLRQYLA | 143 |
| WP_002307203 | 74 | RRRNRILYLQEIFSEEMGKVDDSFFHRLED-SFLV--TEDKR---G ERHPIFGNLE-EEVKYHENFPTIYHLRQYLA | 143 |
| WP_002310390 | 74 | RRRNRILYLQEIFSEEMGKVDDSFFHRLED-SFLV--TEDKR---G ERHPIFGNLE-EEVKYHENFPTIYHLRQYLA | 143 |
| WP_002352408 | 74 | RRRNRILYLQEIFSEEMGKVDDSFFHRLED-SFLV--TEDKR---G ERHPIFGNLE-EEVKYHENFPTIYHLRQYLA | 143 |
| WP_012997688 | 74 | RRRNRILYLQEIFAEEMSKVDDSFFHRLED-SFLV--TEDKR---G ERHPIFGNLE-EEVKYHENFPTIYHLRQYLA | 143 |
| WP_014677909 | 74 | RRRNRILYLQEIFSEEMGKVDDSFFHRLED-SELV--TEDKR---G ERHPIFGNLE-EEVKYHENFPTIYHLRQYLA | 143 |
| WP_019312892 | 74 | RRRNRILYLQEIFAEEMSKVDDSFFHRLED-SFLV--TEDKR---G ERHPIFGNLE-EEVKYHENFPTIYHLRQYLA | 143 |
| WP_019313659 | 74 | RRRNRILYLQEIFSEEMGKVDDSFFHRLED-SFLV--TEDKR---G ERHPIFGNLE-EEVKYHENFPTIYHLRQYLA | 143 |
| WP_019314093 | 74 | RRRNRILYLQEIFSEEMGKVDDSFFHRLED-SFLV--TEDKR---G ERHPIFGNLE-EEVKYHENFPTIYHLRQYLA | 143 |
| WP_019315370 | 74 | RRRNRILYLQEIFSEEMGKVDDSFFHRLED-SELV--TEDKR---G ERHPIFGNLE-EEVKYHENFPTIYHLRQYLA | 143 |
| WP_019803776 | 74 | RRRNRILYLQEIFSEEMGKVDDSFFHRLED-SELV--TEDKR---G ERHPIFGNLE-EEVKYHENFPTIYHLRQYLA | 143 |
| WP_019805234 | 74 | RRRNRILYLQEIFSEEMGKVDDSFFHRLED-SFLV--TEDKR---G ERHPIFGNLE-EEVKYHENFPTIYHLRQYLA | 143 |
| WP_024783594 | 74 | RRRNRILYLQEIFSEEMGKVDDSFFHRLED-SFLV--TEDKR---G ERHPIFGNLE-EEVKYHENFPTIYHLRQYLA | 143 |
| WP_024784288 | 74 | RRRNRILYLQEIFAEEMSKVDDSFFHRLDE-SFLT--DDDKN---F DSHPIFGNKA-EEDAYHQKFPTIYHLRKHLA | 143 |
| WP_024784666 | 74 | RRRNRILYLQEIFAEEMSKVDDSFFHRLED-SFLV--TEDKR---G ERHPIFGNLE-EEVKYHENFPTIYHLRQYLA | 143 |
| WP_024784894 | 74 | RRRNRILYLQEIFSEEMGKVDDSFFHRLED-SFLV--TEDKR---G ERHPIFGNLE-EEVKYHENFPTIYHLRQYLA | 143 |
| WP_024786433 | 74 | RRRNRILYLQEIFAEEMNKVDDSFFHRLDE-SFLT--DDDKN---F DSHPIFGNKA-EEDAYHQKFPTIYHLRKHLA | 143 |
| WP_049473442 | 74 | RRRNRILYLQEIFAEEMSKVDDSFFHRLED-SFLV--TEDKR---G ERHPIFGNLE-EEVKYYENFPTIYHLRQYLA | 143 |
| WP_049474547 | 74 | RRRNRILYLQEIFSEEMGKVDDSFFHRLED-SFLV--TEDKR---G ERHPIFGNLE-EEVKYHENFPTIYHLRQYLA | 143 |
| EMC03581 | 67 | RRRNRILYLQEIFAEEMSKVDDSFFHRLED-SFLV--TEDKR---G ERHPIFGNLE-EEVKYHENFPTIYHLRQYLA | 136 |
| WP_000428612 | 75 | RRKNRLRYLQEIFAEEMSKVDSSFFHRLDD-SFLI--PEDKK---G SKYPIFATLI-EEKEYHKQFPTIYHLRKQLA | 144 |
| WP_000428613 | 75 | RRKNRLRYLQEIFAEEMSKVDSSFFHRLDD-SFLI--PEDKR---G SKYPIFATLI-EEKEYHKQFPTIYHLRKQLA | 144 |
| WP_049523028 | 74 | RRRNRILYLQEIFAAEMNKVDESFFHRLDD-SFLV--PEDKR---G SKYPIFGTLR-EEKEYHKQFPTIYYLRKILA | 143 |
| WP_003107102 | 43 | RRINRIKYLOSIFDDEMSKIDSAFFQRIKD-SFLV--PDDKN---D DRHPIFGNIK-DEVDYHKNYPTIYHLRKKLA | 112 |
| WP_054279288 | 76 | RRKNRLCYLRDIFESEMHTIDKHFFLRLED-SFLH--KSDKR---Y EAHPIFGTLQ-EEKAYHDNYPTIYHLRKALA | 145 |
| WP_049531101 | 75 | RRKNRLRYLQEIFSEEISKVDNSFFHRLDD-SFLV--PEDKR---G SKYPIFATLT-EEKEYYKQFPTIYHLRKQLA | 144 |
| WP_049538452 | 75 | RRKNRLRYLQEIFAEEMNKVDSSFFHRLDD-SFLV--PEDKR---G SKYPIFATLA-EEKEYHKQFPTIYHLRKQLA | 144 |
| WP_049549711 | 75 | RRKNRLRYLQEIFSGEMSKVDSSFFHRLDD-SFLV--PEDKR---G SKYPIFATLV-EEKEYHKQFPTIYHLRKQLA | 144 |
| WP_007896501 | 76 | RRRYRLCQLQNIFATEMVKVDDTFFQRLSE-SFFY--YQDKA---F DKHPIFGNSK-EERAYHKTYPTIYHLRKDLA | 145 |
| EFR44625 | 28 | RRRYRLCQLQNIFATEMVKVDDTFFQRLSE-SFFY--YQDKA---F DKHPIFGNSK-EERAYHKTYPTIYHLRKDLA | 97 |
| WP_002897477 | 74 | RRRNRILYLQEIFTESMNEIDESFFHRLDD-SFLV--PEDKR---G SKYPIFATLQ-EEKEYHKQFPTIYHLRKQLA | 143 |
| WP_002906454 | 74 | RRKNRLRYLQEIFSEEISKLDSSFFHRLDD-SFLV--PEDKR---G SKYIFATLE-EEKEYHKKFPTIYHLRKHLA | 143 |
| WP_009729476 | 75 | RRKNRLRYLQEIFSEEIGKVDSSFFHRLDD-SFLI--PEDKR---G SKYPIFATLA-EEKKYHKQFPTIYHLRKQLA | 144 |
| CQR24647 | 74 | RRRNRILYLQDIFSPELNQVDESFLHRLDD-SFLVa--EDKR---G ERHVIFGNIA-DEVKYHKEFPTIYHLRKHLA | 143 |
| WP_000066813 | 75 | RRKNRLRYLQEIFSQEISKVDSSFFHRLDD-FFLV--PEDKR---G SKYPIFATLV-EEKEYHKKFPTIYHLRKHLA | 144 |
| WP_009754323 | 75 | RRKNRLRYLQEIFAEEMSKVDSSFFHRLDD-SFLV--PEDKS---G SKYPIFATLA-EEKEYHKKFPTIYHLRKHLA | 144 |
| WP_044674937 | 74 | RRRNRILYLQEIFAEEINKIDDSFFQRLDD-SFLIv--EDKQ---G SKHPIFGTLQ-EEKKYHKQFPTIYHLRKQLA | 143 |
| WP_044676715 | 74 | RRRNRILYLQEIFAEEINKIDDSFFQRLDD-SFLIv--EDKQ---G SKHPIFGTLQ-EEKEYHKQFPTIYHLRKQLA | 143 |
| WP_044680361 | 74 | RRRNRILYLQEIFAEEINKIDDSFFQRLDD-SFLIv--EDKQ---G SKHPIFGTLQ-EEKEYHKQFPTIYHLRKQLA | 143 |
| WP_044681799 | 74 | RRRNRILYLQEIFAEEINKIDDSFFQRLDD-SFLIv--EDKQ---G SKHPIFGTLQ-EEKEYHKQFPTIYHLRKQLA | 143 |
| WP_049533112 | 74 | RRRNRLRYLQEIFAEEMNKVDENFFQRLDD-SFLV--DEDKR---G ERHPIFGNIA-AEVKYHDDFPTIYHLRKHLA | 143 |
| WP_029090905 | 28 | HRKFRLRLLEDMFEKEILSKDPSFFIRLKE-AFLSpkDEQKQ---F ----LFNDKDyTDADYYEQYKTIYHLRYDLI | 100 |
| WP_006506696 | 61 | KRRERIRLLRAILQDMVLEKDPTFFIRLEHtSFLD--EEDKAky1G DNYNLFIDEDfNDYTYYHKYPTIYHLRKALC | 139 |
| AIT42264 | 74 | RRKNRICYLQEIFSNEMAKVDDSFFHRLEE-SFLV--EEDKK---H ERHPIFGNIV-DEVAYHEKYPTIYHLRKKLV | 143 |
| WP_034440723 | 73 | RRRFRIRELQKIFDKSMGEVDSNFFHRLDE-SFLV--EEDKE---Y SKYPIFSNEK-EDKNYYDKYPTIYHLRKDLA | 142 |
| AKQ21048 | 74 | RRKNRICYLQEIFSNEMAKVDDSFFHRLEE-SFLV--EEDKK---H ERHPIFGNIV-DEVAYHEKYPTIYHLRKKLV | 143 |
| WP_004636532 | 74 | RRRNRLIYLQDIFQQPMLAIDENFFHRLDD-SFFV--PDDKS---Y DRHPIFGSLE-EEVAYHNTYPTIYHLRKHLA | 143 |
| WP_002364836 | 74 | RRRNRLRYLQAFFEEAMTDLDENFFARLQE-SFLV--PEDKK---W HRHPIFAKLE-DEVAYHETYPTIYHLRKKLA | 143 |
| WP_016631044 | 25 | RRRNRLRYLQAFFEEAMTDLDENFFARLQE-SFLV--PEDKK---W HRHPIFAKLE-DEVAYHETYPTIYHLRKKLA | 94 |
| EMS75795 | | ---------------------------------------------- ------------------------------- | |
| WP_002373311 | 74 | RRRNRLRYLQAFFEEAMTDLDENFFARLQE-SFLV--PEDKK---W HRHPIFAKLE-DEVAYHETYPTIYHLRKKLA | 143 |
| WP_002378009 | 74 | RRRNRLRYLQAFFEEAMTALDENFFARLQE-SFLV--PEDKK---W HRHPIFAKLE-DEVAYHETYPTIYHLRKKLA | 143 |
| WP_002407324 | 74 | RRRNRLRYLQAFFEEAMTALDENFFARLQE-SFLV--PEDKK---W HRHPIFAKLE-DEVAYHETYPTIYHLRKKLA | 143 |
| WP_002413717 | 74 | RRRNRLRYLQAFFEEAMTDLDENFFARLQE-SFLV--PEDKK---W HRHPIFAKLE-DEVAYHETYPTIYHLRKKLA | 143 |
| WP_010775580 | 74 | RRRNRLRYLQAFFEEAMTALDENFFARLQE-SFLV--PEDKK---W HRHPIFAKLE-DEVAYHETYPTIYHLRKKLA | 143 |
| WP_010818269 | 74 | RRRNRLRYLQAFFEEAMTALDENFFARLQE-SFLV--PEDKK---W HRHPIFAKLE-DEVAYHETYPTIYHLRKKLA | 143 |
| WP_010824395 | 74 | RRRNRLRYLQAFFEEAMTDLDENFFARLQE-SFLV--PEDKK---W HRHPIFAKLE-DEVAYHETYPTIYHLRKKLA | 143 |
| WP_016622645 | 74 | RRRNRLRYLQAFFEEAMTALDENFFARLQE-SFLV--PEDKK---W HRHPIFAKLE-DEVAYHETYPTIYHLRKKLA | 143 |
| WP_033624816 | 74 | RRRNRLRYLQAFFEEAMTALDENFFARLQE-SFLV--PEDKK---W HRHPIFAKLE-DEVAYHETYPTIYHLRKKLA | 143 |
| WP_033625576 | 74 | RRRNRLRYLQAFFEEAMTALDENFFARLQE-SFLV--PEDKK---W HRHPIFAKLE-DEVAYHETYPTIYHLRKKLA | 143 |
| WP_033789179 | 74 | RRRNRLRYLQAFFEEAMTALDENFFARLQE-SFLV--PEDKK---W HRHPIFAKLE-DEVAYHETYPTIYHLRKKLA | 143 |
| WP_002310644 | 74 | RRRQRILELQKIFAPEILKIDEHFFARLNE-SFLV--LDEKK---Q SRHPVFATIK-QWKSYHQTYPTIYHLRQALA | 143 |
| WP_002312694 | 74 | RRRQRILELOKIFAPEILKIDEHFFARLNE-SELV--PDEKK---Q SRHPVFATIK-QWKSYHQTYPTIYHLRQALA | 143 |
| WP_002314015 | 74 | RRRQRILELQKIFAPEILKIDEHFFARLNE-SELV--LDEKK---Q SRHPVFATIK-QWKSYHQTYPTIYHLRQALA | 143 |
| WP_002320716 | 74 | RRRQRILELQKIFAPEILKIDEHFFARLNE-SFLV--LDEKK---Q SRHPVFATIK-QWKSYHQTYPTIYHLRQALA | 143 |
| WP_002330729 | 74 | RRRQRILELQKIFAPEILKIDEHFFARLNE-SELV--LDEKK---Q SRHPVFATIK-QWKSYHQTYPTIYHLRQALA | 143 |
| WP_002335161 | 74 | RRRQRILELQKIFAPEILKIDEHFFARLNE-SFLV--LDEKK---Q SRHPVFATIK-QWKSYHQTYPTIYHLRQALA | 143 |
| WP_002345439 | 74 | RRRQRILELQKIFAPEILKIDEHFFARLNE-SFLV--LDEKK---Q SRHPVFATIK-QWKSYHQTYPTIYHLRQALA | 143 |
| WP_034867970 | 74 | RRKYRLSKLQDLFAEELCKQDDCFFVRLEE-SFLV--PEEKQ---Y KPASIFPTLE-EEKEYYQKYPTIYHLRQKLV | 143 |
| WP_047937432 | 74 | RRRQRILELQKIFAPEILKIDEHFFARLNE-SFLV--LDEKK---Q SRHPVFATIK-QWKSYHQTYPTIYHLRQALA | 143 |
| WP_010720994 | 74 | RRKYRLSKLQDLFAEELCKQDDCFFVRLEE-SFLV--PEEKQ---Y KPASIFPTLE-EEKEYYQKYPTIYHLRQKLV | 143 |
| WP_010737004 | 74 | RRKYRLSKLQDLFAEELCKQDDCFFVRLEE-SFLV--PEEKQ---Y KPASIFPTLE-EEKEYYQKYPTIYHLRQKLV | 143 |
| WP_034700478 | 74 | RRKYRLSKLQDLFAEELCKQDDCFFVRLEE-SFLV--PEEKQ---Y KPASIFPTLE-EEKEYYQKYPTIYHLRQKLV | 143 |
| WP_007209003 | 74 | RRKNRICYLQEIFQPEMNHLDNNFFYRLNE-SFLVa--DDAK---Y DKHPIFGTLD-EEIHGHEQFPTIYHLRKYLA | 143 |
| WP_023519017 | 74 | RRRQRVLALQDIFAEEIHKKDPNFFARLEE-GDRV--EADKR---F AKFPVFATLS-EEKNYHRQYPTIYHLRHDLA | 143 |
| WP_010770040 | 74 | RRRNRICRLQDLFTEEMNQVDANFFHRLQE-SFLV--PDEKE---F ERHAIFGKME-EEVSYYREFPTIYHLRKHLA | 143 |
| WP_048604708 | 74 | RRRQRISYLQTFFQEEMNRIDPNFFNRLDE-SFLI--EEDKL---S ERHPIFGTIE-EEVAYHKNYATIYHLRKELA | 143 |
| WP_010750235 | 74 | RRKYRILELQKIFSEEILKKDSHFFARLDE-SFLI--PEDKQ---Y ARFPIFPTLL-EEKAYYQNYPTIYHLRQKLA | 143 |
| AII16583 | 113 | RRKNRICYLQEIFSNEMAKVDDSFFHRLEE-SFLV--EEDKK---H ERHPIFGNIV-DEVAYHEKYPTIYHLRKKLV | 182 |
| WP_029073316 | 66 | KRRERIRLLRGIMEDMVLDVDPTFFIRLANVSFLD--QEDKKdylK SNYNLFIDKDfNDKTYYDKYPTIYHLRKHLC | 144 |
| WP_031589969 | 66 | KRRERIRLLREIMEDMVLDVDPTFFIRLANVSFLD--QEDKKdylK DVYPIFGKRE-EELLYHDTHKTIYHLRSELA | 144 |
| KDA45870 | 75 | RRKNRLRYLQEIFAPALAKVDPNFFYRLEE-SSLVa--EDKK---Y DVYPIFGKRE-EELLYHDTHKTIYHLRSELA | 144 |
| WP_039099354 | 62 | RRKWRLGLLREIFEPYITPVDDTFFLRKKQ-SNLS--PKDQR---K -QTSLFNDRT--DRAFYDDYPTIYHLRYKLM | 132 |
| AKP02966 | 65 | RRKNRINWLNEIFSEELANTDPSFLIRLON-SWVSkkDPDRK---R DKYNLFIDNPyTDKEYYREFPTIFHLRKELI | 137 |
| WP_010991369 | 74 | RRRNRISYLQGIFAEEMSKTDANFFCRLSD-SFYV--DNEKR---N SRHPFFATIE-EEVEYHKNYPTIYHLREELV | 143 |
| WP_033838504 | 74 | RRRNRISYLQGIFAEEMSKTDANFFCRLSD-SFYV--DNEKR---N SRHPFFATIE-EEVEYHKNYPTIYHLREELV | 143 |
| EHN60060 | 77 | RRRNRISYLQGIFAEEMSKTDANFFCRLSD-SFYV--DNEKR---N SRHPFFATIE-EEVEYHKNYPTIYHLREELV | 146 |
| EFR89594 | | ---------------------------------------------- ------------------------------- | |
| WP_038409211 | 74 | RRRNRIAYLQEIFAAEMAEVDANFFYRLED-SFYI--ESEKR---H SRHPFFATIE-EEVAYHEEYKTIYHLREKLV | 143 |
| EFR95520 | | ---------------------------------------------- ------------------------------- | |
| WP_003723650 | 74 | RRRNRISYLQEIFAVEMANIDANFFCRLND-SFYV--DSEKR---N SRHPFFATIE-EEVAYHDNYRTIYHLREKLV | 143 |
| WP_003727705 | 74 | RRRNRISYLQEIFAVEMANIDANFFCRLND-SFYV--DSEKR---N SRHPFFATIE-EEVAYHKNYRTIYHLREELV | 143 |
| WP_003730785 | 74 | RRRNRISYLQEIFAVEMANIDANFFCRLND-SFYV--DSEKR---N SRHPFFATIE-EEVAYHKNYRTIYHLREELV | 143 |
| WP_003733029 | 74 | RRRNRISYLQEIFAIQMNEVDDNFFNRLKE-SFYA--ESDKK---Y NRHPFFGTVE-EEVAYYKDFPTIYHLRKELI | 143 |
| WP_003739838 | 74 | RRRNRISYLQEIFALEMANIDANFFCRLND-SFYV--DSEKR---N SRHPFFATIE-EEVAYHKNYRTIYHLREELV | 143 |
| WP_014601172 | 74 | RRRNRISYLQEIFAVEMANIDANFFCRLND-SFYV--DSEKR---N SRHPFFATIE-EEVAYHKNYRTIYHLREELV | 143 |
| WP_023548323 | 74 | RRRNRISYLQEIFAVEMANIDANFFCRLND-SFYV--DSEKR---N SRHPFFATIE-EEVAYHKNYRTIYHLREELV | 143 |
| WP_031665337 | 74 | RRRNRISYLQEIFAVEMANIDANFFCRLND-SFYV--DSEKR---N SRHPFFATIE-EEVAYHKNYRTIYHLREELV | 143 |
| WP_031669209 | 74 | RRRNRISYLQEIFAIQMNEVDDNFFNRLKE-SFYA--ESDKK---Y NRHPFFGTVE-EEVAYYKDFPTIYHLRKELI | 143 |
| WP_033920898 | 74 | RRRNRISYLQEIFAVEMANIDANFFCRLND-SFYV--DSEKR---N SRHPFFATIE-EEVAYHKNYRTIYHLREELV | 143 |
| AKI42028 | 77 | RRRNRISYLQEIFAVEMANIDANFFCRLND-SFYV--DSEKR---N SRHPFFATIE-EEVAYHKNYRTIYHLREELV | 146 |
| AKI50529 | 77 | RRRNRISYLQEIFAVEMANIDANFFCRLND-SFYV--DSEKR---N SRHPFFATIE-EEVAYHKNYRTIYHLREELV | 146 |
| EFR83390 | | ---------------------------------------------- ------------------------------- | |
| WP_046323366 | 74 | RRRNRISYLQEIFTAEMFEVDANFFYRLED-SFYI--ESEKR---Q SRHPFFATIE-EEVAYHENYRTIYHLREKLV | 143 |
| AKE81011 | 90 | RRKNRICYLQEIFSNEMAKVDDSFFHRLEE-SFLV--EEDKK---H ERHPIFGNIV-DEVAYHEKYPTIYHLRKKLV | 159 |
| CUO82355 | 65 | KRRERIRLLRAILQDMVLEKDPTFFIRLEHtSFLD--EEDKAky1G DNYNLFIDEDANDYTYYHKYPTIYHLRKALC | 143 |
| WP_033162887 | 66 | KRRERIRLLRDLLGDMVMEVDPTFFIRLLNVSFLD--EEDKQkn1G DNYNLFIEKDfNDKTYYDKYPTIYHLRKELC | 144 |
| AGZ01981 | 107 | RRKNRICYLQEIFSNEMAKVDDSFFHRLEE-SFLV--EEDKK---H ERHPIFGNIV-DEVAYHEKYPTIYHLRKKLV | 176 |
| AKA60242 | 74 | RRKNRICYLQEIFSNEMAKVDDSFFHRLEE-SFLV--EEDKK---H ERHPIFGNIV-DEVAYHEKYPTIYHLRKKLV | 143 |
| AKS40380 | 74 | RRKNRICYLQEIFSNEMAKVDDSFFHRLEE-SFLV--EEDKK---H ERHPIFGNIV-DEVAYHEKYPTIYHLRKKLV | 143 |
| 4UN5_B | 78 | RRKNRICYLQEIFSNEMAKVDDSFFHRLEE-SFLV--EEDKK---H ERHPIFGNIV-DEVAYHEKYPTIYHLRKKLV | 147 |
| WP_010922251 | 144 | 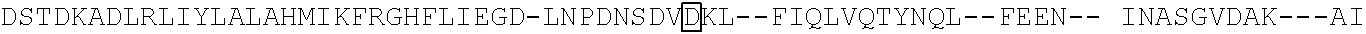 | 211 |
| WP_039695303 | 145 | DSSEKADLRLVYLALAHMIKFRGHFLIEGE-LNAENTDVQKI--FADFVGVYNRT--FDDS-H LSEITVDVA---SI | 212 |
| WP_045635197 | 144 | DSKEKTDLRLIYLALAHMIKYRGHFLYEEA-FDIKNNDIQKI--FNEFISIYDNT--FEGS-S LSGQNAQVE---AI | 211 |
| 5AXW_A | 105 | EEEFSA-------ALLHLAKRRG---VHNV------NEVE------------EDT----GN-- --------E----- | 134 |
| WP_009880683 | | --------------------------------------------------------------- -------------- | |
| WP_010922251 | 144 | DSTDKADLRLIYLALAHMIKFRGHFLIEGD-LNPDNSDVDKL--FIQLVQTYNQL--FEEN-- INASGVDAK---AI | 211 |
| WP_011054416 | 144 | DSTDKVDLRLIYLALAHMIKFRGHFLIEGD-LNPDNSDVDKL--FIQLVQTYNQL--FEEN-- INASRVDAK---AI | 211 |
| WP_011284745 | 144 | DSTDKADLRLIYLALAHMIKFRGHFLIEGD-LNPDNSDVDKL--FIQLVQTYNQL--FEEN-- INASGVDAK---AI | 211 |
| WP_011285506 | 144 | DSTDKADLRLIYLALAHMIKFRGHFLIEGD-LNPDNSDVDKL--FIQLVQTYNQL--FEEN-- INASGVDAK---AI | 211 |
| WP_011527619 | 144 | DSTDKADLRLIYLALAHMIKFRGHFLIEGD-LNPDNSDVDKL--FIQLVQTYNQL--FEEN-- INASGVDAK---AI | 211 |
| WP_012560673 | 144 | DSTDKADLRLIYLALAHMIKFRGHFLIEGD-LNPDNSDVDKL--FIQLVQTYNQL--FEEN-- INASRVDAK---AI | 211 |
| WP_014407541 | 144 | DSTDKADLRLIYLALAHMIKFRGHFLIEGD-LNPDNSDVDKL--FIQLVQIYNQL--FEEN-- INASRVDAK---AI | 211 |
| WP_020905136 | 144 | DSTDKADLRLIYLALAHMIKFRGHFLIEGD-LNPDNSDVDKL--FIQLVQTYNQL--FEEN-- INASGVDAK---AI | 211 |
| WP_023080005 | 144 | DSTDKVDLRLIYLALAHMIKFRGHFLIEGD-LNPDNSDVDKL--FIQLVQTYNQL--FEEN-- INASRVDAK---AI | 211 |
| WP_023610282 | 144 | DSTDKVDLRLIYLALAHMIKFRGHFLIEGD-LNPDNSDVDKL--FIQLVQTYNQL--FEEN-- INASRVDAK---AI | 211 |
| WP_030125963 | 144 | DSTDKADLRLIYLALAHMIKFRGHFLIEGD-LNPDNSDVDKL--FIQLVQTYNQL--FEEN-- INASGVDAK---AI | 211 |
| WP_030126706 | 144 | DSTDKVDLRLIYLALAHMIKFRGHFLIEGD-LNPDNSDVDKL--FIQLVQTYNQL--FEEN-- INASRVDAK---AI | 211 |
| WP_031488318 | 144 | DSTDKADLRLIYLALAHMIKFRGHFLIEGD-LNPDNSDVDKL--FIQLVQTYNQL--FEEN-- INASRVDAK---AI | 211 |
| WP_032460140 | 144 | DSTDKADLRLIYLALAHMIKFRGHFLIEGD-LNPDNSDVDKL--FIQLVQTYNQL--FEEN-- INASRVDAK---AI | 211 |
| WP_032461047 | 144 | DSTDKADLRLIYLALAHMIKFRGHFLIEGG-LNPDNSDVDKL--FIQLVQTYNQL--FEEN-- INASRVDAK---AI | 211 |
| WP_032462016 | 144 | DSTDKADLRLIYLALAHMIKFRGHFLIEGD-LNPDNSDVDKL--FIQLVQTYNQL--FEEN-- INANGVDAK---AI | 211 |
| WP_032462936 | 144 | DSTDKADLRLIYLALAHMIKFRGHFLIEGD-LNPDNSDVDKL--FIQLVQTYNQL--FEEN-- INASRVDAK---AI | 211 |
| WP_032464890 | 144 | DSTDKADLRLIYLALAHMIKFRGHFLIEGD-LNPDNSDVDKL--FIQLVQTYNQL--FEEN-- INASGVDAK---AI | 211 |
| WP_033888930 | 1 | ---------------------------------PDNSDVDKL--FIQLVQTYNQL--FEEN-- INASGVDAK---AI | 36 |
| WP_038431314 | 144 | DSTDKADLRLIYLALAHMIKFRGHFLIEGD-LNPDNSDVDKL--FIQLVQTYNQL--FEEN-- INASRVDAK---AI | 211 |
| WP_038432938 | 144 | DSTDKVDLRLIYLALAHMIKFRGHFLIEGD-LNPDNSDVDKL--FIQLVQTYNQL--FEEN-- INASRVDAK---AI | 211 |
| WP_038434062 | 144 | DSTDKADLRLIYLALAHMIKFRGHFLIEGD-LNPDNSDVDKL--FIQLVQTYNQL--FEEN-- INASRVDAK---AI | 211 |
| BAQ51233 | 55 | DSTDKADLRLIYLALAHMIKFRGHFLIEGD-LNPDNSDVDKL--FIQLVQTYNQL--FEEN-- INASGVDAK---AI | 122 |
| KGE60162 | | --------------------------------------------------------------- -------------- | |
| KGE60856 | | --------------------------------------------------------------- -------------- | |
| WP_002989955 | 144 | DSTDKADLRLIYLALAHMIKFRGHFLIEGD-LNPDNSDVDKL--FIQLVQTYNQL--FEEN-- INASGVDAK---AI | 211 |
| WP_003030002 | 144 | DISQKADLRLVYLALAHMIKFRGHFLIEGQ-LKAENTNVQAL--FKDFVEVYDKT--VEES-H LSEMTVDAL---SI | 211 |
| WP_003065552 | 147 | DSSEKADLRLVYLALAHMIKERGHFLIEGE-LNAENTDVQKI--FADFVGVYDRT--FDDS-H LSEITVDAA---SI | 214 |
| WP_001040076 | 144 | DKKEKADLRLVYLALAHIIKFRGHFLIEDDrFDVRNTDIQKQ--YQAFLEIFDTT--FENN-D LLSQDVDVE---AI | 212 |
| WP_001040078 | 144 | DKQEKADLRLIYLALAHIIKFRGHFLIEDDrFDVRNTDIQKQ--YQAFLEIFDTT--FENN-H LLSQNVDVE---AI | 212 |
| WP_001040080 | 144 | DKKEKADLRLIYIALAHIIKFRGHFLIEDDsFDVRNTDISKQ--YQDFLEIFNIT--FENN-D LLSQNVDVE---AI | 212 |
| WP_001040081 | 144 | DKKEKADLRLIYIALAHIIKFRGHFLIEDDsFDVRNTDISKQ--YQDFLEIFNTI--FENN-D LLSQNVDVE---AI | 212 |
| WP_001040083 | 144 | DKKEKADLRLIYIALAHIIKFRGHFLIEDDsFDVRNTDISKQ--YQDFLEIFNTT--FENN-D LLSQNVDVE---AI | 212 |
| WP_001040085 | 144 | DKKEKADLRLIYIALAHIIKFRGHFLIEDDsFDVRNTDISKQ--YQDFLEIFNTT--FENN-D LLSQNVDVE---AI | 212 |
| WP_001040087 | 144 | DKKEKADLRLIYIALAHIIKFRGHFLIEDDsFDVRNTDISKQ--YQDFLEIFNTT--FENN-D LLSQNVDVE---AI | 212 |
| WP_001040088 | 144 | DKKEKADLRLIYIALAHIIKFRGHFLIEDDsFDVRNTDISKQ--YQDFLEIFNTT--FENN-D LLSQNVDVE---AI | 212 |
| WP_001040089 | 144 | DKKEKADLRLIYIALAHIIKERGHFLIEDDsFDVRNTDISKQ--YQDFLEIFNTT--FENN-D LLSQNVDVE---AI | 212 |
| WP_001040090 | 144 | DKKEKADLRLIYIALAHIIKFRGHFLIEDDsFDVRNTDISKQ--YQDFLEIFNTT--FENN-D LLSQNVDVE---AI | 212 |
| WP_001040091 | 144 | DKKEKADLRLIYIALAHIIKFRGHFLIEDDsFDVRNTDISKQ--YQDFLEIFNTT--FENN-D LLSQNVDVE---AI | 212 |
| WP_001040092 | 144 | DKKEKADLRLVYLALAHIIKFRGHFLIEDDrFDVRNTDIQKQ--YQAFLEIFDTS--FENN-H LLSQNVDVE---AI | 212 |
| WP_001040094 | 144 | DKKEKADLRLIYLALAHIIKFRGHFLIEDDrFDVRNTDIQKQ--YQAFLEIFDTT--FENN-D LLSQNVDVE---AI | 212 |
| WP_001040095 | 144 | DKKEKADLRLIYLALAHIIKFRGHFLIEDDrFDVRNTDIQKQ--YQAFLEIFDTT--FENN-D LLSQNVDVE---AI | 212 |
| WP_001040096 | 144 | DKKEKADLRLIYLALAHIIKFRGHFLIEDDrFDVRNTDIQKQ--YQAFLEIFDTT--FENN-D LLSQNVDVE---AI | 212 |
| WP_001040097 | 144 | DKKEKADLRLIYLALAHIIKFRGHFLIEDDrFDVRNTDIQKQ--YQAFLEIFDTT--FENN-D LLSQNVDVE---AI | 212 |
| WP_001040098 | 144 | DKKEKADLRLIYLALAHIIKFRGHFLIEDDrFDVRNTDIQKQ--YQAFLEIFDTT--FENN-D LLSQNVDVE---AI | 212 |
| WP_001040099 | 144 | DKKEKADLRLIYLALAHIIKFRGHFLIEDDrFDVRNTDIQKQ--YQAFLEIFDTT--FENN-D LLSQNVDVE---AI | 212 |
| WP_001040100 | 144 | DKKEKADLRLIYLALAHIIKFRGHFLIEDDrFDVRNTDIQKQ--YQAFLEIFDTT--FENN-D LLSQNVDVE---AI | 212 |
| WP_001040104 | 144 | DKKEKADLRLIYIALAHIIKFRGHFLIEDDsFDVRNTDISKQ--YQDFLEIFNTT--FENN-D LLSQNVDVE---AI | 212 |
| WP_001040105 | 144 | DKKEKADLRLIYIALAHIIKFRGHFLIEDDsFDVRNTDISKQ--YQDFLEIFNTT--FENN-D LLSQNVDVE---AI | 212 |
| WP_001040106 | 144 | DKKEKANLRLVYLALAHIIKFRGHFLIEDDsFDVRNTDIQRQ--YQAFLEIFDTT--FENN-H LLSQNIDVE---GI | 212 |
| WP_001040107 | 144 | DKKEKADLRLVYLALAHIIKFRGHFLIEDDsFDVRNTDIQRQ--YQAFLEIFDIT--FENN-H LLSQNIDVE---GI | 212 |
| WP_001040108 | 144 | DKKEKADLRLVYLALAHIIKFRGHFLIEDDsFDVRNTDIQRQ--YQAFLEIFDTT--FENN-H LLSQNIDVE---GI | 212 |
| WP_001040109 | 144 | DKKEKANLRLVYLALAHIIKFRGHFLIEDDsFDVRNTDIQRQ--YQAFLEIFDTT--FENN-H LLSQNIDVE---GI | 212 |
| WP_001040110 | 144 | DKKEKANLRLVYLALAHIIKFRGHFLIEDDsFDVRNTDIQRQ--YQAFLEIFDIT--FENN-H LLSQNIDVE---GI | 212 |
| WP_015058523 | 144 | DKKEKADLRLVYLALAHIIKFRGHFLIEDDrFDVRNTDIQKQ--YQAFLEIFDTS--FENN-H LLSQNVDVE---AI | 212 |
| WP_017643650 | 144 | DKKEKADLRLIYLALAHIIKFRGHFLIEDDrFDVRNTDIQKQ--YQAFLEIFDTT--FENN-D LLSQNVDVE---AI | 212 |
| WP_017647151 | 144 | DKKEKADLRLFYLALAHIIKFRGHFLIEDDsFDVRNTDIQRQ--YQAFLEIFDTT--FENN-H LLSQNIDIE---GI | 212 |
| WP_017648376 | 144 | DKKEKADLRLFYLALAHIIKFRGHFLIEDDsFDVRNTDIQRQ--YQAFLEIFDTT--FENN-H LLSQNIDVE---GI | 212 |
| WP_017649527 | 144 | DKKEKADLRLIYIALAHIIKFRGHFLIEDDsFDVRNTDISKQ--YQDFLEIFNTT--FENN-D LLSQNVDVE---AI | 212 |
| WP_017771611 | 144 | DKKEKADLRLVYLALAHIIKFRGHFLIEDDsFDVRNTDIQRQ--YQAFLEIFDTT--FENN-H LLSQNIDVE---GI | 212 |
| WP_017771984 | 144 | DKKEKADLRLIYIALAHIIKFRGHFLIEDDsFDVRNTDISKQ--YQDFLEIFNTT--FENN-D LLSQNVDVE---AI | 212 |
| CFQ25032 | 144 | DKKEKADLRLIYIALAHIIKFRGHFLIEDDsFDVRNTDISKQ--YQDFLEIFNTT--FENN-D LLSQNVDVE---AI | 212 |
| CFV16040 | 144 | DKKEKADLRLIYIALAHIIKFRGHFLIEDDsFDVRNTDISKQ--YQDFLEIFNTT--FENN-D LLSQNVDVE---AI | 212 |
| KLJ37842 | 144 | DKKEKADLRLIYIALAHIIKFRGHFLIEDDsFDVRNTDISKQ--YQDFLEIFNTT--FENN-D LLSQNVDVE---AI | 212 |
| KLJ72361 | 144 | DKKEKADLRLIYIALAHIIKFRGHFLIEDDsFDVRNTDISKQ--YQDFLEIFNTT--FENN-D LLSQNVDVE---AI | 212 |
| KLL20707 | 144 | DKKEKADLRLIYIALAHIIKFRGHFLIEDDsFDVRNTDISKQ--YQDFLEIFNTT--FENN-D LLSQNVDVE---AI | 212 |
| KLL42645 | 144 | DKKEKANLRLVYLALAHIIKFRGHFLIEDDsFDVRNTDIQRQ--YQAFLEIFDTT--FENN-H LLSQNIDVE---GI | 212 |
| WP_047207273 | 144 | DKKEKADLRLIYIALAHIIKFRGHFLIEDDsFDVRNTDISKQ--YQDFLEIFNTT--FENN-D LLSQNVDVE---AI | 212 |
| WP_047209694 | 144 | DKKEKADLRLIYLALAHIIKFRGHFLIEDDsFDVRNTDIQKQ--YQAFLEIFDTT--FENN-D LLSQNVDVE---AI | 212 |
| WP_050198062 | 144 | DKKEKADLRLIYIALAHIIKFRGHFLIEDDsFDVRNTDISKQ--YQDFLEIFNTT--FENN-D LLSQNVDVE---AI | 212 |
| WP_050201642 | 144 | DKKEKADLRLIYIALAHIIKFRGHFLIEDDsFDVRNTDISKQ--YQDFLEIFNTT--FENN-D LLSQNVDVE---AI | 212 |
| WP_050204027 | 144 | DKKEKANLRLVYLALAHIIKFRGHFLIEDDsFDVRNTDIQRQ--YQAFLEIFDTT--FENN-H LLSQNIDVE---GI | 212 |
| WP_050881965 | 144 | DKKEKADLRLIYIALAHIIKFRGHFLIEDDsFDVRNTDISKQ--YQDFLEIFNTT--FENN-D LLSQNVDVE---AI | 212 |
| WP_050886065 | 144 | DKKEKADLRLIYIALAHIIKFRGHFLIEDDsFDVRNTDISKQ--YQDFLEIFNTT--FENN-D LLSQNVDVE---AI | 212 |
| AHN30376 | 144 | DKKEKADLRLVYLALAHIIKFRGHFLIEDDrFDVRNTDIQKQ--YQAFLEIFDTS--FENN-H LLSQNVDVE---AI | 212 |
| EAO78426 | 144 | DKKEKADLRLIYIALAHIIKFRGHFLIEDDsFDVRNTDISKQ--YQDFLEIFNTT--FENN-D LLSQNVDVE---AI | 212 |
| CCW42055 | 144 | DKKEKADLRLVYLALAHIIKFRGHFLIEDDrFDVRNTDIQKQ--YQAFLEIFDTT--FENN-H LLSQNVDVE---AI | 212 |
| WP_003041502 | 144 | DISQKADLRLVYLALAHMIKFRGHFLIEGQ-LKAENTNVQAL--FKDFVEVYDKT--VEES-H LSEITVDAL---SI | 211 |
| WP_037593752 | 145 | NSKEKADLRLVYLALAHMIKFRGHFLYEGD-LKAENTDVQAL--FKDFVEEYDKT--IEES-H LSEITVDAL---SI | 212 |
| WP_049516684 | 145 | DISQKADLRLVYLALAHMIKFRGHFLIEGQ-LKAENTNVQAL--FKDFVEVYDKT--VEES-H LSEMTVDAL---SI | 212 |
| GAD46167 | 144 | NSKEKADLRLVYLALAHMIKFRGHFLYEGD-LKAENTDVQAL--FKDFVEEYDKT--IEES-H LSEITVDAL---SI | 211 |
| WP_018363470 | 145 | DSTEKADLRLVYLALAHMIKFRGHFLIEGE-LNAENTDVQKL--FTDFVGVYDRT--FDDS-H LSEITVDAA---SI | 212 |
| WP_003043819 | 144 | DSPEKADLRLIYLALAHIIKFRGHFLIEGK-LNAENSDVAKL--FYQLIQTYNQL--FEES-- LDEIEVDAK---GI | 211 |
| WP_006269658 | 144 | DTSKKADLRLVYLALAHMIKFRGHFLYEGD-LKAENTDVQAL--FKDFVEEYDKT--IEES-H LSEITVDAL---SI | 211 |
| WP_048800889 | 144 | DSTGKVDLRLVYLALAHMIKFRGHFLIEGQ-LKAENTDVQTL--FNDFVEVYDKT--IEES-H LAEITVDAL---SI | 211 |
| WP_012767106 | 144 | DSTDKADLRLIYLALAHMIKFRGHFLIEGD-LNPDNSDMDKL--FIQLVQTYNQL--FEEN-- INASRVDAK---AI | 211 |
| WP_014612333 | 144 | DSTDKADLRLIYLALAHMIKFRGHFLIEGD-LNPDNSDVDKL--FIQLVQTYNQL--FEEK-- INASGVDAK---AI | 211 |
| WP_015017095 | 144 | DSTDKADLRLIYLALAHMIKFRGHFLIEGD-LNPDNSDMDKL--FIQLVQTYNQL--FEEN-- INASRVDAK---AI | 211 |
| WP_015057649 | 144 | DSTDKADLRLIYLALAHMIKFRGHFLIEGD-LNPDNSDMDKL--FIQLVQTYNQL--FEEN-- INASRVDAK---AI | 211 |
| WP_048327215 | 144 | DSTDKADLRLIYLALAHMIKFRGHFLIEGD-LNPDNSDMDKL--FIQLVQTYNQL--FEEN-- INASRVDAK---AI | 211 |
| WP_049519324 | 144 | DSTDKADLRLIYLALAHMIKFRGHFLIEGD-LNPDNSDVDKL--FIQLVQTYNQL--FEEN-- INASRVDAK---AI | 211 |
| WP_012515931 | 144 | DNPQKADLRLIYLAVAHIIKFRGHFLIEGT-LSSKNNNLQKS--FDHLVDTYNLL--FEEQ-- LLTEGINAK---EL | 211 |
| WP_021320964 | 144 | DNPQKADLRLIYLAVAHIIKFRGHFLIEGT-LSSKNNNLQKS--FDHLVDTYNLL--FEEQ-- LLTEGINAK---EL | 211 |
| WP_037581760 | 144 | DNPQKADLRLIYLAVAHIIKFRGHFLIEGT-LSSKNNNLQKS--FDHLVDTYNLL--FEEQ-- LLTEGINAK---EL | 211 |
| WP_004232481 | 144 | DSPEKVDLRLVYLALAHMIKFRGHFLIEGQ-LNAENTDVQKI--FADFVGVYDRT--FDDS-H LSEITVDAA---SI | 211 |
| WP_009854540 | 145 | DSSEKADLRLVYLALAHMIKYRGHFLIEGK-LNAENTDVOKL--FTDFVGVYDRT--FDDS-H LSEITVDVA---ST | 212 |
| WP_012962174 | 145 | DSHEKADLRLIYLALAHMIKFRGHFLIEGE-LNAENTDVQKL--FEAFVEVYDRT--FDDS-N LSEITVDAS---SI | 212 |
| WP_039695303 | 145 | DSSEKADLRLVYLALAHMIKFRGHFLIEGE-LNAENTDVOKI--FADFVGVYNRT--FDDS-H LSEITVDVA---SI | 212 |
| WP_014334983 | 144 | DSQEKADLRLVYLALAHMIKYRGHFLIEGE-LNAENTDVQKL--FNVFVETYDKI--VDES-H LSEIEVDAS---SI | 211 |
| WP_003099269 | 144 | DSDQKADLRLIYLALAHIIKFRGHFLIEGN-LDSENTDVHVL--FLNLVNIYNNL--FEED-- VETASIDAE---KI | 211 |
| AHY15608 | 144 | DSDQKADLRLIYLALAHIIKFRGHFLIEGN-LDSENTDVHVL--FLNLVNIYNNL--FEED-- VETASIDAE---KI | 211 |
| AHY17476 | 144 | DSDQKADLRLIYLALAHIIKFRGHFLIEGN-LDSENTDVHVL--FLNLVNIYNNL--FEED-- VETASIDAE---KI | 211 |
| ESR09100 | | --------------------------------------------------------------- -------------- | |
| AGM98575 | 144 | DSDQKADLRLIYLALAHIIKFRGHFLIEGN-LDSENTDVHVL--FLNLVNIYNNL--FEED-- VETASIDAE---KI | 211 |
| ALF27331 | 144 | DNPEKTDLRLVYLALAHIIKFRGHFLIEGK-FDTRNNDVORL--FQEFLAVYDNT--FENS-S LQEQNVQVE---EI | 211 |
| WP_018372492 | 144 | DTPDKMDIRLIYLALAHIIKYRGHFLIEGD-LDIENIGIQDS--FKSFIEEYNTQ--FGTK-- -LDSTTKVE---AI | 209 |
| WP_045618028 | 145 | DSKEKADFRLIYLALAHIIKYRGHFLYEES-FDIKNNDIQKI--FNEFISIYDNT--FEGS-S LNGQNAQVE---AI | 212 |
| WP_045635197 | 144 | DSKEKTDLRLIYLALAHMIKYRGHFLYEEA-FDIKNNDIQKI--FNEFISIYDNT--FEGS-S LSGQNAQVE---AI | 211 |
| WP_002263549 | 144 | DNPEKVDLRLVYLALAHIIKFRGHFLIEGK-FDTRNNDVQRL--FQEFLAVYDNT--FENS-S LQEQNVQVE---EI | 211 |
| WP_002263887 | 144 | DNPEKVDLRLVYLALAHIIKERGHFLIEGK-FDTRNNDVQRL--FQEFLAVYDNT--FENS-S LQEQNVQVE---EI | 211 |
| WP_002264920 | 144 | DSTEKADLRLVYLALAHMIKFRGHFLIEGE-LNAENTDVQKL--FADFVGVYDRT--FDDS-H LSEITVDAS---SI | 211 |
| WP_002269043 | 144 | DNPEKTDLRLVYLALAHIIKFRGHFLIEGK-FDTRNNDVORL--FQEFLAVYDNT--FENS-S LQEQNVQVE---EI | 211 |
| WP_002269448 | 144 | DNPEKVDLRLVYLALAHIIKFRGHFLIEGK-FDTRNNDVORL--FQEFLAVYDNT--FENS-S LQEQNVQVE---EI | 211 |
| WP_002271977 | 144 | DNPEKVDLRLVYLALAHIIKFRGHFLIEGK-FDTRNNDVORL--FQEFLAVYDNT--FENS-S LQEQNVQVE---EI | 211 |
| WP_002272766 | 144 | DNPEKVDLRLVYLALAHIIKFRGHFLIEGK-FDTRNNDVORL--FQEFLAVYDNT--FENS-S LQEQNVQVE---EI | 211 |
| WP_002273241 | 144 | DNPEKVDLRLVYLALAHIIKFRGHFLIEGK-FDTRNNDVORL--FQEFLAVYDNT--FENS-S LQEQNVQVE---EI | 211 |
| WP_002275430 | 144 | DNPEKVDLRLVYLALAHIIKFRGHFLIEGK-FDTRNNDVQRL--FQEFLAVYDNT--FENS-S LQEQNVQVE---EI | 211 |
| WP_002276448 | 144 | DNPEKVDLRLVYLALAHIIKFRGHFLIEGK-FDTRNNDVQRL--FQEFLAVYDNT--FENS-S LQEQNVQVE---EI | 211 |
| WP_002277050 | 144 | DSTEKADLRLVYLALAHMIKFRGHFLIEGE-LNAENTDVQKL--FADFVGVYDRT--FDDS-H LSEITVDAS---SI | 211 |
| WP_002277364 | 144 | DNPEKVDLRLVYLALAHIIKFRGHFLIEGK-FDTRNNDVQRL--FQEFLAVYDNT--FENS-S LQEQNVQVE---EI | 211 |
| WP_002279025 | 144 | DNPEKVDLRLVYLALAHIIKFRGHFLIEGK-FDTRNNDVORL--FQEFLAVYDNT--FENS-S LQEQNVQVE---EI | 211 |
| WP_002279859 | 144 | DSTEKADLRLVYLALAHMIKFRGHFLIEGE-LNAENTDVQKL--FADFVGVYDRT--FDDS-H LSEITVDAS---SI | 211 |
| WP_002280230 | 144 | DNPEKVDLRLVYLALAHIIKERGHFLIEGK-FDTRNNDVORL--FQEFLAVYDNT--FENS-S LQEQNVQVE---E1 | 211 |
| WP_002281696 | 144 | DNPEKVDLRLVYLALAHIIKFRGHFLIEGK-FDTRNNDVORL--FQEFLAVYDNT--FENS-S LQEQNVQVE---EI | 211 |
| WP_002282247 | 144 | DSTEKADLRLVYLALAHMIKFRGHFLIEGE-LNAENTDVQKL--FADFVGVYDRT--FDDS-H LSEITVDAS---SI | 211 |
| WP_002282906 | 144 | DNPEKVDLRLVYLALAHIIKFRGHFLIEGK-FDTRNNDVQRL--FQEFLAVYDNT--FENS-S LQEQNVQVE---EI | 211 |
| WP_002283846 | 144 | DNPEKTDLRLVYLALAHIIKFRGHFLIEGK-FDTRNNDVORL--FQEFLAVYDNT--FENS-S LQEQNVQVE---EI | 211 |
| WP_002287255 | 144 | DNPEKVDLRLVYLALAHIIKFRGHFLIEGK-FDTRNNDVORL--FQEFLAVYDNT--FENS-S LQEQNVQVE---EI | 211 |
| WP_002288990 | 144 | DNPEKTDLRLVYLALAHIIKFRGHFLIEGK-FDTRNNDVORL--FQEFLAVYDNT--FENS-S LQEQNVQVE---EI | 211 |
| WP_002289641 | 144 | DNPEKTDLRLVYLALAHIIKFRGHFLIEGK-FDTRNNDVORL--FQEFLAVYDNT--FENS-S LQEQNVQVE---EI | 211 |
| WP_002290427 | 144 | DNPEKTDLRLVYLALAHIIKFRGHFLIEGK-FDTRNNDVQRL--FQEFLAVYDNT--FENS-S LQEQNVQVE---EI | 211 |
| WP_002295753 | 144 | DNPEKVDLRLVYLALAHIIKFRGHFLIEGK-FDTRNNDVQRL--FQEFLAVYDNT--FENS-S LQEQNVQVE---EI | 211 |
| WP_002296423 | 144 | DNPEKTDLRLVYLALAHIIKFGGHFLIEGK-FDTRNNDVORL--FQEFLAVYDNT--FENS-S LQEQNVQVE---EI | 211 |
| WP_002304487 | 144 | NSTEKADLRLVYLSLAHMIKFRGHFLIEGQ-LKAENTNVQAL--FKDFVEVYDKT--VEES-H LSEMTVDAL---SI | 211 |
| WP_002305844 | 144 | DNPEKVDLRLVYLALAHIIKFRGHFLIEGK-FDTRNNDVORL--FQEFLAVYDNT--FENS-S LQEQNVQVE---EI | 211 |
| WP_002307203 | 144 | DNPEKVDLRLVYLALAHIIKFRGHFLIEGK-FDTRNNDVQKL--FQEFLAVYDNT--FENS-S LQEQNVQVE---EI | 211 |
| WP_002310390 | 144 | DNPEKTDLRLVYLALAHIIKFRGHFLIEGK-FDTRNNDVQRL--FQEFLAVYDNT--FENS-S LQEQNVQVE---EI | 211 |
| WP_002352408 | 144 | DNPEKTDLRLVYLALAHIIKFRGHFLIEGK-FDTRNNDVORL--FQEFLAVYDNT--FENS-S LQEQNVQVE---EI | 211 |
| WP_012997688 | 144 | DNPEKVDLRLVYLALAHIIKFRGHFLIEGK-FDTRNNDVORL--FQEFLAVYDNT--FENS-S LQEQNVQVE---EI | 211 |
| WP_014677909 | 144 | DNPEKTDLRLVYLALAHIIKFRGHFLIEGK-FDTRNNDVORL--FQEFLAVYDNT--FENS-S LQEQNVQVE---EI | 211 |
| WP_019312892 | 144 | DNPEKVDLRLVYLALAHIIKFRGHFLIEGK-FDTRNNDVORL--FQEFLAVYDNT--FENS-S LQEQNVQVE---EI | 211 |
| WP_019313659 | 144 | DNPEKVDLRLVYLALAHIIKFRGHFLIEGK-FDTRNNDVQRL--FQEFLAVYDNT--FENS-S LQEQNVQVE---EI | 211 |
| WP_019314093 | 144 | DNPEKVDLRLVYLALAHIIKFRGHFLIEGK-FDTRNNDVQKL--FQEFLAVYDNT--FENS-S LQEQNVQVE---EI | 211 |
| WP_019315370 | 144 | DNPEKTDLRLVYLALAHIIKFRGHFLIEGK-FDTRNNDVORL--FQEFLAVYDNT--FENS-S LQEQNVQVE---EI | 211 |
| WP_019803776 | 144 | DNPEKVDLRLVYLALAHIIKFRGHFLIEGK-FDTRNNDVORL--FQEFLAVYDNT--FENS-S LQEQNVQVE---EI | 211 |
| WP_019805234 | 144 | DNPEKTDLRLVYLALAHIIKFRGHFLIEGK-FDTRNNDVQRL--FQEFLAVYDNT--FENS-S LQEQNVQVE---EI | 211 |
| WP_024783594 | 144 | DNPEKVDLRLVYLALAHIIKERGHFLIEGK-FDTRNNDVORL--FQEFLAVYDNT--FENS-S LQEQNVQVE---EI | 211 |
| WP_024784288 | 144 | DSTEKADLRLVYLALAHMIKFRGHFLIEGE-LNAENTDVQKL--FADFVGVYDRT--FDDS-H LSEITVDAS---SI | 211 |
| WP_024784666 | 144 | DNPEKVDLRLVYLALAHIIKFRGHFLIEGK-FDTRNNDVQRL--FQEFLAVYDNT--FENS-S LQEQNVQVE---EI | 211 |
| WP_024784894 | 144 | DNPEKTDLRLVYLALAHIIKFRGHFLIEGK-FDTRNNDVQRL--FQEFLAVYDNT--FENS-S LQEQNVQVE---EI | 211 |
| WP_024786433 | 144 | DSTEKADLRLVYLALAHMIKFRGHFLIEGE-LNAENTDVQKL--FADFVGVYDRT--FDDS-H LSEITVDAS---SI | 211 |
| WP_049473442 | 144 | DNPEKVDLRLVYLALAHIIKFRGHFLIEGK-FDTRNNDVORL--FQEFLAVYDNT--FENS-S LQEQNVQVE---EI | 211 |
| WP_049474547 | 144 | DNPEKTDLRLVYLALAHIIKFRGHFLIEGK-FDTRNNDVQRL--FQEFLAVYDNT--FENS-S LQEQNVQVE---EI | 211 |
| EMC03581 | 137 | DNPEKVDLRLVYLALAHIIKFRGHFLIEGK-FDTRNNDVQRL--FQEFLAVYDNT--FENS-S LQEQNVQVE---EI | 204 |
| WP_000428612 | 145 | DSKEKTDLRLIYLALAHMIKYRGHFLYEDT-FDIKNNDIQKI--FNEFISIYNNT--FEGN-S LSGQNVQVE---AI | 212 |
| WP_000428613 | 145 | DSKEKTDLRLIYLALAHMIKYRGHFLYEDT-FDIKNNDIQKI--FSEFISIYDNT--FEGS-S LSGQNAQVE---AI | 212 |
| WP_049523028 | 144 | DSKEKVDLRLIYLALAHIIKYRGHFLYEDS-FDIKNNDIQKI--FNEFTILYDNT--FEES-S LSKGNAQVE---EI | 211 |
| WP_003107102 | 113 | DSDEKADLRLIYLALAHIIKFRGHFLIEGD-LDSQNTDVNAL--FLKLVDTYNLM--FEDD-- IDTQTIDAT---VI | 180 |
| WP_054279288 | 146 | DNTEKADLRLIYLALAHIIKFRGHFLIEGA-LSANNTDVQQL--VHALVDAYNIM--FEED-- LDIEAIDVK---AI | 213 |
| WP_049531101 | 145 | DSKEKADLRLIYLTLAHMIKYRGHFLYEES-FDIKNNDIQKI--FNEFISIYDNT--FEGS-S LSGQNAQVE---AI | 212 |
| WP_049538452 | 145 | DSKEKADLRLIYLALAHMIKYRGHFLYEEA-FDIKNNDIQKI--FNEFINIYDNT--FEGS-S LSGONEQVE---AI | 212 |
| WP_049549711 | 145 | DSKEKADLRLIYLVLAHMIKYRGHFLYEEA-FDIKNNDIQKI--FNEFISIYDNT--FEGS-S LSGQNAQVE---TI | 212 |
| WP_007896501 | 146 | DRDQKADLRLIYLALSHIIKFRGHFLIEGK-LNSENTDVQKL--FIALVTVYNLL--FEEE-- IAGETCDAK---AL | 213 |
| ERF44625 | 98 | DRDQKADLRLIYLALSHIIKFRGHFLIEGK-LNSENTDVQKL--FIALVTVYNLL--FEEE-- IAGETCDAK---AL | 165 |
| WP_002897477 | 144 | DSKEKSDVRLIYLALAHMIKYRGHFLYEET-FDIKNNDIQKI--FNEFINIYDNT--FEGS-S LSGQNAQVE---AI | 211 |
| WP_002906454 | 144 | DSKEKTDLRLIYLALAHMIKYRGHFLYEES-FDIKNNDIQKI--FNEFISIYDNT--FEGS-S LSGQNAQVE---AI | 211 |
| WP_009729476 | 145 | DSKEKTDLRLIYLALAHMIKYRGHFLYEEA-FDIKNNDIQKI--FNEFISIYNNT--FEGN-S LSGQNVQVE---AI | 212 |
| CQR24647 | 144 | DSSEKADLRLVYLALAHIIKYRGHFLIDEP-IDIRNMNSQNL--FKEFLLAFDGI--QVDC-Y LASKHTDIS---GI | 211 |
| WP_000066813 | 145 | DSKEKTDLRLIYLALAHMIKYRGHFLYEES-FDIKNNDIQKI--FSEFISIYDNT--FEGK-S LSGQNAQVE---AI | 212 |
| WP_009754323 | 145 | DSKEKADLRLIYLALAHITKYRGHFLYEEA-FDIKNNDIQKI--FNEFINIYDNT--FEGS-S LSGONAQVE---AI | 212 |
| WP_044674937 | 144 | DSSQKADIRLIYLALAHIIKYRGHFLFEGD-LKSENKDVQHL--FNDFVEMFDKY--VEGS-Y LSENLPNVA---DV | 211 |
| WP_044676715 | 144 | DSSQKADIRLIYLALAHIIKYRGHFLFEGD-LKSENKDVQHL--FNDFVEMFDKT--VEGS-Y LSENLPNVA---DV | 211 |
| WP_44680361 | 144 | DSSQKADIRLIYLALAHIIKYRGHFLFEGD-LKSENKDVQHL--FNDFVEMFDKT--VEGS-Y LSENLPNVA---DV | 211 |
| WP_044681799 | 144 | DSSQKADIRLIYLALAHIIKYRGHFLFEGD-LKSENKDVQHL--FNDFVEMFDKT--VEGS-Y LSENLPNVA---DV | 211 |
| WP_049533112 | 144 | DISQKADLRLVYLALAHMIKFRGHFLIEGQ-LKAENTNVQAL--FKDFVEVYDKT--VEES-H LSEMTVDAL---SI | 211 |
| WP_029090905 | 101 | SQHRQFDIREVYLAIHHLIKYRGHFIYEDQtFTTDGNQLQHH--IKAIITMINSTl---NR-- IIPETIDINvfeKI | 171 |
| WP_006506696 | 140 | ESTEKADPRLIYLALHHIVKYRGNFLYEGQkFNMDASNIEDK--LSDIFTQFTSFnnIPYEdD --KKNLEIL---EI | 210 |
| AIT42264 | 144 | DSTDKADLRLIYLALAHMIKFRGHFLIEGD-LNPDNSDVDKL--FIQLVQTYNQL--FEEN-- INASGVDAK---AI | 211 |
| WP_034440723 | 143 | DSNQKADLRLIYLALAHMIKYRGHFLIEGD-LKMDFISISES--FQEFIDSYNEVcaLEDE-N NDELLTQIE---NI | 217 |
| AKQ21048 | 144 | DSTDKADLRLIYLALAHMIKFRGHLFIEGD-LNPDNSDVDKL--FIQLVQTYNQL--FEEN-- INASGVDAK---AI | 211 |
| WP_004636532 | 144 | DNPEKADLRLVYTALAHIVKYRGHFLIEGE-LNTENTSISET--FEQFLDTYDSI--FKEQ-- LVGDISKVE---EI | 210 |
| WP_002364836 | 144 | DSSEQADLRLIYLALAHIVKYRGHFLIEGK-LSTENISVKEQ--FQQFMIIYNQT--FVNGeS PLPESVLIE---EE | 217 |
| WP_016631044 | 95 | DSSEQADLRLIYLALAHIVKYRGHFLIEGK-LSTENTSVKDQ--FQQFMVIYNQT--FVNGeS PLPESVLIE---EE | 168 |
| EMS75795 | | --------------------------------------------------------------- -------------- | |
| WP_002737311 | 144 | DSSEQADLRLIYLALAHIVKYRGHFLIEGK-LSTENTSVKDQ--FQQFMVIYNQT--FVNGeS PLPESVLIE---EE | 217 |
| WP_002378009 | 144 | DSSEQADLRLIYLALAHIVKYRGHFLIEGK-LSTENISVKEQ--FQQFMIIYNQT--FVNGeS PLPESVLIE---EE | 217 |
| WP_002407324 | 144 | DSSEQADLRLIYLALAHIVKYRGHFLIEGK-LSTENISVKEQ--FQQFMIIYNQT--FVNGeS PLPESVLIE---EE | 217 |
| WP_002413717 | 144 | DSSEQADLRLIYLALAHIVKYRGHFLIEGK-LSTENISVKEQ--FQQFMIIYNQT--FVNGeS PLPESVLIE---EE | 217 |
| WP_010775580 | 144 | DSSEQADLRLIYLALAHIVKYRGHFLIEGK-LSTENISVKEQ--FQQFMIIYNQT--FVNGeS PLPESVLIE---EE | 217 |
| WP_010818269 | 144 | DSSEQADLRLIYLALAHIVKYRGHFLIEGK-LSTENISVKEQ--FQQFMIIYNQT--FVNGeS PLPESVLIE---EE | 217 |
| WP_010824395 | 144 | DSSEQADLRLIYLALAHIVKYRGHFLIEGK-LSTENTSVKDQ--FQQFMVIYNQT--FVNGeS PLPESVLIE---EE | 217 |
| WP_016622645 | 144 | DSSEQADLRLIYLALAHIVKYRGHFLIEGK-LSTENISVKEK--FQOFMIIYNQT--FVNGeG PLPESVLIE---EE | 217 |
| WP_033624816 | 144 | DSSEQADLRLIYLALAHIVKYRGHFLIEGK-LSTENISVKDQ--FQQFMVIYNQT--FVNGeS PLPESVLIE---EE | 217 |
| WP_033625576 | 144 | DSSEQADLRLIYLALAHIVKYRGHFLIEGK-LSTENISVKEQ--FQOFMIIYNQT--FVNGeS PLPESVLIE---EE | 217 |
| WP_033789179 | 144 | DSSEQADLRLIYLALAHIVKYRGHFLIEGK-LSTENISVKEQ--FQQFMIIYNQT--FVNGeS PLPESVLIE---EE | 217 |
| WP_002310644 | 144 | DSSEKADIRLVYLAMAHLLKYRGHFLIEGE-LNTENSSVTET--FRQFLSTYNQQ--FSEA-D KLDEAVDCS---FV | 216 |
| WP_002312694 | 144 | DSSEKADIRLVYLAMAHLLKYRGHFLIEGE-LNTENSSVTET--FRQFLSTYNQQ--FSEA-G KLDEAVDCS---FV | 216 |
| WP_002314015 | 144 | DSSEKADIRLVYLAMAHLLKYRGHFLIEGE-LNTENSSVTET--FRQFLSTYNQQ--FSEA-D KLDEAVDCS---FV | 216 |
| WP_002320716 | 144 | DSSEKADIRLVYLAMAHLLKYRGHFLIEGE-LNTENSSVTET--FROFLSTYNQQ--FSEA-D KLDEAVDCS---FV | 216 |
| WP_002330729 | 144 | DSSEKADIRLVYLAMAHLLKYRGHFLIEGE-LNTENSSVTET--FRQFLSTYNQQ--FSEA-D KLDEAVDCS---FV | 216 |
| WP_002335161 | 144 | DSSEKADIRLVYLAMAHLLKYRGHFLIEGE-LNTENSSVTET--FRQFLSTYNQQ--FSEA-D KLDEAVDCS---FV | 216 |
| WP_002345439 | 144 | DSSEKADIRLVYLAMAHLLKYRGHFLIEGE-LNTENSSVTET--FRQFLSTYNQQ--FSEA-D KLDEAVDCS---FV | 216 |
| WP_034867970 | 144 | DSTEKEDLRLVYLALAHLLKYRGHELFEGD-LDTENTSIEES--FRVFLEQYSKQ--SDOP-- -LIVHQPVL---TI | 209 |
| WP_047937432 | 144 | DSSEKADIRLVYLAMAHLLKYRGHFLIEGE-LNTENSSVTET--FRQFLSTYNQQ--FSEA-D KLDEAVDCS---FV | 216 |
| WP_010720994 | 144 | DSTEKGDLRLVYLALAHLLKYRGHELFEGD-LDTENTSIEES--FRVFLEQYGKQ--SDQP-- -LIVHQPVL---TI | 209 |
| WP_010737004 | 144 | DSTEKEDLRLVYLALAHLLKYRGHFLFEGD-LDTENTSIEES--FRVFLEQYSKQ--SDQP-- -LIVHQPVL---TI | 209 |
| WP_034700478 | 144 | DSTEKEDLRLVYLALAHLLKYRGHELFEGD-LDTENTSIEES--FRVFLEQYGKQ--SDQP-- -LIVHQPVL---TI | 209 |
| WP_007209003 | 144 | DGDEKADLRLVYLAIAHIIKFRGNFLIEGE-LNTENNSVIELs--KVFVQLYNQT1-SELE-- FIDESIDES---EV | 214 |
| WP_023519017 | 144 | NSKEQADIRLVYLAIAHCLKYRGHFLFEGE-LDTENTSVTEN--YQQFLQAYQQF--FPEP-- -IGDLDDAV---PI | 209 |
| WP_010770040 | 144 | DTSEQADLRLVYLALAHIVKYRGHFLIEGE-LNTENSSVSET--FRTFIQVYNQI--FRENe- PLAVPDNIE---EL | 212 |
| WP_048604708 | 144 | DAEEKADLRLVYLALAHIIKYRGHFLIEGR-LSTENTSTEET--FKTFLQKYNQT--FN---- PVDETISIG---SI | 208 |
| WP_010750235 | 144 | DSTEKADIRLVYLALAHMIKYRGHFLFEGE-LDTENTSVEET--FKEFIDIYNEQ--FEEG-- -IIFYKDIP---LI | 209 |
| AII16583 | 183 | DSTDKADLRLIYLALAHMIKFRGHFLIEGD-LNPDNSDVDKL--FIQLVQTYNQL--FEEN-- INASGVDAK---AI | 250 |
| WP_029073316 | 145 | ESKEKEDPRLIYLALHHIVKYRGNFLYEGQKFSMDVSNIEDK--MIDVLRQFNEIn1FEYVeD --KKIDEVL---NV | 215 |
| WP_031589969 | 145 | ESKEKEDPRLIYLALHHIVKYRGNFLYEGQKFSMDVSNIEDK--MIDVLRQFNEIn1FEYVeD --KKIDEVL---NV | 215 |
| KDA45870 | 145 | NNDRPADLRLVYLALAHIIKYRGNFLLEGE-IDLRTTDINKV--FAEFSETLNEN--SDENIG ----KLDVA---DI | 209 |
| WP_039099354 | 133 | TEKRQFDIREIYLAMHHIVKYRGHFLNEAPvSSFKSSEINLVahFDRLNTIFADL--FSESgF -TDKLAEVK---AL | 206 |
| AKP02966 | 138 | INKNKADIRLVYLALHNILKYRGNFTYEHQkFNISTLNSNLS---KELIELNQQLikYDIS-- -FPDNCDWNhisDI | 208 |
| WP_010991369 | 144 | NSSEKADLRLVYLALAHIIKYRGNFLIEGA-LDTQNTSVDGI--YKQFIQTYNQV--FASGIE KLEDNKDVA---KI | 217 |
| WP_033838504 | 144 | NSSEKADLRLVYLALAHIIKYRGNFLIEGA-LDTQNTSVDGI--YKQFIQTYNQV--FASGIE KLEDNKDVA---KI | 217 |
| EHN60060 | 147 | NSSEKADLRLVYLALAHIIKYRGNFLIEGA-LDTQNTSVDGI--YKQFIQTYNQV--FASGIE KLEDNKDVA---KI | 220 |
| EFR89594 | | --------------------------------------------------------------- -------------- | |
| WP_038409211 | 144 | NSSDKADLRLVYLALAHIIKYRGNFLIEGM-LDTKNTSVDEV--FKQFIQTYNQI--FASDIE RLEENKEVA---EI | 217 |
| EFR95520 | | --------------------------------------------------------------- -------------- | |
| WP_003723650 | 144 | NSSEKADLRLVYLALAHIIKYRGNFLIEGA-LDTKNTSVDEV--YKQFIETYNQV--FMSNIE KVEENIEVA---NI | 217 |
| WP_003727705 | 144 | NSSEKADLRLVYLALAHIIKYRGNFLIEGA-LDTKNTSVDEV--YKQFIQTYNQV--FMSNIE KVEENTEVA---SI | 217 |
| WP_003730785 | 144 | NSSEKADLRLVYLALAHIIKYRGNFLIEGA-LDTKNTSVDEV--YKQFIQTYNQV--FMSNIE KVEENTEVA---SI | 217 |
| WP_003733029 | 144 | DSQKKADLRLVYLALAHIIKYRGHFLIEGA-LDTKNTSIDEM--FKQFLQIYNQV--FANDIE KTEKNQEVA---QI | 217 |
| WP_003739838 | 144 | NSSEKADLRLVYLALAHIIKYRGNFLIEGA-LDTKNTSVDGV--YKQFIQTYNQV--FISNIE KMEENTTVA---DI | 217 |
| WP_014601172 | 144 | NSSEKADLRLVYLALAHIIKYRGNFLIEGA-LDTKNTSVDGV--YEQFIQTYNQV--FMSNIE KVEENIEVA---NI | 217 |
| WP_023548323 | 144 | NSSEKADLRLVYLALAHIIKYRGNFLIEGA-LDTKNTSVDGV--YEQFILTYNQV--FMSNIE KVEENIEVA---NI | 217 |
| WP_031665337 | 144 | NSSEKADLRLVYLALAHIIKYRGNFLIEGA-LDTKNTSVDGV--YEQFIQTYNQV--FMSNIE KVEENIEVA---NI | 217 |
| WP_031669209 | 144 | DSQKKADLRLVYLALAHIIKYRGHFLIEGA-LDTKNTSIDEM--FKQFLQIYNQV--FANDIE KTEKNQEVA---QI | 217 |
| WP_033920898 | 144 | NSSEKADLRLVYLALAHIIKYRGNFLIEGA-LDTKNTSVDGV--YEQFIQTYNQV--FMSNIE KVEENIEVA---NI | 217 |
| AKI42028 | 147 | NSSEKADLRLVYLALAHIIKYRGNFLIEGA-LDTKNTSVDGV--YEQFIQTYNQV--FMSNIE KVEENIEVA---NI | 220 |
| AKI50529 | 147 | NSSEKADLRLVYLALAHIIKYRGNFLIEGA-LDTKNTSVDGV--YEQFIQTYNQV--FMSNIE KVEENIEVA---NI | 220 |
| EFR83390 | | --------------------------------------------------------------- -------------- | |
| WP_046323366 | 144 | NSSDKADLRLVYLALAHIIKYRGNFLIEGK-LDTKNTSVDEV--FKQFIKTYNQV--FASDIE RIEENNEVA---KI | 217 |
| AKE81011 | 160 | DSTDKADLRLIYLALAHMIKFRGHFLIEGD-LNPDNSDVDKL--FIQLVQTYNQL--FEEN-- INASGVDAK---AI | 227 |
| CUO82355 | 144 | ESTEKADPRLIYLALHHIVKYRGNFLYEGQKFNMDASNIEDK--LSDVFTQFADFnnIPYEdD --KKNLEIL---EI | 214 |
| WP_033162887 | 145 | ENKEKADPRLIYLALHHIVKYRGNFLYEGQSFTMDNSDIEER--LNSAIEKFMSIneFDNRiV --SDINSMI---AV | 215 |
| AGZ01981 | 177 | DSTDKADLRLIYLALAHMIKFRGHFLIEGD-LNPDNSDVDKL--FIQLVQTYNQL--FEEN-- INASGVDAK---AI | 244 |
| AKA60242 | 144 | DSTDKADLRLIYLALAHMIKFRGHFLIEGD-LNPDNSDVDKL--FIQLVQTYNQL--FEEN-- INASGVDAK---AI | 211 |
| AKS40380 | 144 | DSTDKADLRLIYLALAHMIKFRGHFLIEGD-LNPDNSDVDKL--FIQLVQTYNQL--FEEN-- INASGVDAK---AI | 211 |
| 4UN5_B | 148 | DSTDKADLRLIYLALAHMIKFRGHFLIEGD-LNPDNSDVDKL--FIQLVQTYNQL--FEEN-- INASGVDAK---AI | 215 |
| WP_010922251 | 212 | 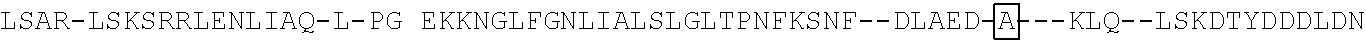 | 277 |
| WP_039695303 | 213 | LTEK-ISKSRRLENLIKY-Y-PT EKKNTLFGNLIALALGLQPNFKTNF--KLSED-A---KLQ--FSKDTYEEDLEE | 278 |
| WP_045635197 | 212 | FTDK-ISKSAKRERVLKL-F-PD EKSTGLFSEFLKLIVGNQADFKKHF--DLEDK-A---PLQ--FSKDTYDEDLEN | 277 |
| 5AXW_A | 135 | LSTK--------EQISRN-S--K ----------------------------LEEKyVa--ELQ-------------- | 157 |
| WP_009880683 | | --------------------------------------------------------------- -------------- | |
| WP_010922251 | 212 | LSAR-LSKSRRLENLIAQ-L-PG EKKNGLFGNLIALSLGLTPNFKSNF--DLAED-A---KLQ--LSKDTYDDDLDN | 277 |
| WP_011054416 | 212 | LSAR-LSKSRRLENLIAQ-L-PG EKKNGLFGNLIALSLGLTPNFKSNF--DLAED-A---KLQ--LSKDTYDDDLDN | 277 |
| WP_011284745 | 212 | LSAR-LSKSRRLENLIAQ-L-PG EKKNGLFGNLIALSLGLTPNFKSNF--DLAED-A---KLQ--LSKDTYDDDLDN | 277 |
| WP_011285506 | 212 | LSAR-LSKSRRLENLIAQ-L-PG EKKNGLFGNLIALSLGLTPNFKSNF--DLAED-A---KLQ--LSKDTYDDDLDN | 277 |
| WP_011527619 | 212 | LSAR-LSKSRRLENLIAQ-L-PG EKRNGLFGNLIALSLGLTPNFKSNF--DLAED-A---KLQ--LSKDTYDDDLDN | 277 |
| WP_012560673 | 212 | LSAR-LSKSRRLENLIAQ-L-PG EKRNGLFGNLIALSLGLTPNFKSNF--DLAED-A---KLQ--LSKDTYDDDLDN | 277 |
| WP_014407541 | 212 | LSAR-LSKSRRLENLIAQ-L-PG EKKNGLFGNLIALSLGLTPNFKSNF--DLAED-A---KLQ--LSKDTYDDDLDN | 277 |
| WP_020905136 | 212 | LSAR-LSKSRRLENLIAQ-L-PG EKKNGLFGNLIALSLGLTPNFKSNF--DLAED-A---KLQ--LSKDTYDDDLDN | 277 |
| WP_023080005 | 212 | LSAR-LSKSRRLENLIAQ-L-PG EKKNGLFGNLIALSLGLTPNFKSNF--DLAED-A---KLQ--LSKDTYDDDLDN | 277 |
| WP_023610282 | 212 | LSAR-LSKSRRLENLIAQ-L-PG EKKNGLFGNLIALSLGLTPNFKSNF--DLAED-A---KLQ--LSKDTYDDDLDN | 277 |
| WP_030125963 | 212 | LSAR-LSKSRRLENLIAQ-L-PG EKKNGLFGNLIALSLGLTPNFKSNF--DLAED-A---KLQ--LSKDTYDDDLDN | 277 |
| WP_030126706 | 212 | LSAR-LSKSRRLENLIAQ-L-PG EKRNGLFGNLIALSLGLTPNFKSNF--DLAED-A---KLQ--LSKDTYDDDLDN | 277 |
| WP_031488318 | 212 | LSAR-LSKSRRLENLIAQ-L-PG EKRNGLFGNLIALSLGLTPNFKSNF--DLAED-A---KLQ--LSKDTYDDDLDN | 277 |
| WP_032460140 | 212 | LSAR-LSKSRRLENLIAQ-L-PG EKRNGLFGNLIALSLGLTPNFKSNF--DLAED-A---KLQ--LSKDTYDDDLDN | 277 |
| WP_032461047 | 212 | LSAR-LSKSRRLENLIAQ-L-PG EKKNGLFGNLIALSLGLTPNFKSNF--DLAED-A---KLQ--LSKDTYDDDLDN | 277 |
| WP_032462016 | 212 | LSAR-LSKSRRLENLIAQ-L-PG EKKNGLFGNLIALLLGLTPNFKSNF--DLAED-A---KLQ--LSKDTYDDDLDN | 277 |
| WP_032462936 | 212 | LSAR-LSKSRRLENLIAQ-L-PG EKKNGLFGNLIALSLGLTPNFKSNF--DLAED-A---KLQ--LSKDTYDDDLDN | 277 |
| WP_032464890 | 212 | LSAR-LSKSRRLENLIAQ-L-PG EKKNGLFGNLIALSLGLTPNFKSNF--DLAED-A---KLQ--LSKDTYDDDLDN | 277 |
| WP_033888930 | 37 | LSAR-LSKSRRLENLIAQ-L-PG EKKNGLFGNLIALSLGLTPNFKSNF--DLAED-A---KLQ--LSKDTYDDDLDN | 102 |
| WP_038431314 | 212 | LSAR-LSKSRRLENLIAQ-L-PG EKKNGLFGNLIALSLGLTPNFKSNF--DLAED-T---KLQ--LSKDTYDDDLDN | 277 |
| WP_038432938 | 212 | LSAR-LSKSRRLENLIAQ-L-PG EKRNGLFGNLIALSLGLTPNFKSNF--DLAED-A---KLQ--LSKDTYDDDLDN | 277 |
| WP_038434062 | 212 | LSAR-LSKSRRLENLIAQ-L-PG EKKNGLFGNLIALSLGLTPNFKSNF--DLAED-A---KLQ--LSKDTYDDDLDN | 277 |
| BAQ51233 | 123 | LSAR-LSKSRRLENLIAQ-L-PG EKKNGLFGNLIALSLGLTPNFKSNF--DLAED-A---KLQ--LSKDTYDDDLDN | 188 |
| KGE60162 | | --------------------------------------------------------------- -------------- | |
| KGE60856 | | --------------------------------------------------------------- -------------- | |
| WP_002989955 | 212 | LSAR-LSKSRRLENLIAQ-L-PG EKKNTLFGNLIALALGLOPNFKMNF--KLSED-A---KLQ--FSKDSYEEDLGE | 277 |
| WP_003030002 | 212 | LTEK-VSKSRRLENLIAH-Y-PA QKSTGIFAEFLKLIVGNQADFKKHF--NLEDK-T---PLQ--FAKDSYDEDLEN | 277 |
| WP_003065552 | 215 | LTEK-ISKSRRLENLIKY-Y-PT QKSTGIFAEFLKLIVGNQADFKKHF--NLEDK-T---PLQ--FAKDSYDEDLEN | 280 |
| WP_001040076 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040078 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040080 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040081 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040083 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040085 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040087 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040088 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040089 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040090 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040091 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040092 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_001040094 | 213 | LTDK-ISKSAKKDRILAR-Y-PN QKSTGIFAEFLKLIVGNQADFKKHF--NLEDK-T---PLQ--FAKDSYDEDLEN | 278 |
| WP_001040095 | 213 | LTDK-ISKSAKKDRILAR-Y-PN QKSTGIFAEFLKLIVGNQADFKKHF--NLEDK-T---PLQ--FAKDSYDEDLEN | 278 |
| WP_001040096 | 213 | LTDK-ISKSAKKDRILAR-Y-PN QKSTGIFAEFLKLIVGNQADFKKHF--NLEDK-T---PLQ--FAKDSYDEDLEN | 278 |
| WP_001040097 | 213 | LTDK-ISKSAKKDRILAR-Y-PN QKSTGIFAEFLKLIVGNQADFKKHF--NLEDK-T---PLQ--FAKDSYDEDLEN | 278 |
| WP_001040098 | 213 | LTDK-ISKSAKKDRILAR-Y-PN QKSTGIFAEFLKLIVGNQADFKKHF--NLEDK-T---PLQ--FAKDSYDEDLEN | 278 |
| WP_001040099 | 213 | LTDK-ISKSAKKDRILAR-Y-PN QKSTGIFAEFLKLIVGNQADFKKHF--NLEDK-T---PLQ--FAKDSYDEDLEN | 278 |
| WP_001040100 | 213 | LTDK-ISKSAKKDRILAR-Y-PN QKSTGIFAEFLKLIVGNQADFKKHF--NLEDK-T---PLQ--FAKDSYDEDLEN | 278 |
| WP_001040104 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040105 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_001040106 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_001040107 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_001040108 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_001040109 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_001040110 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_015058523 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_017643650 | 213 | LTDK-ISKSAKKDRILAR-Y-PN QKSTGIFAEFLKLIVGNQADFKKHF--NLEDK-T---PLQ--FAKDSYDEDLEN | 278 |
| WP_017647151 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_017648376 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_017649527 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_017771611 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_017771984 | 213 | LTDK-ISKSAKKDRILAQ-Y-PN QKSTGIFAEFLKLIVGNQADFKKYF--NLEDK-T---PLQ--FAKDSYDEDLEN | 278 |
| CFQ25032 | 213 | LTDK-ISKSAKKDRILAQ-Y-PN QKSTGIFAEFLKLIVGNQADFKKYF--NLEDK-T---PLQ--FAKDSYDEDLEN | 278 |
| CFV16040 | 213 | LTDK-ISKSAKKDRILAQ-Y-PN QKSTGIFAEFLKLIVGNQADFKKYF--NLEDK-T---PLQ--FAKDSYDEDLEN | 278 |
| KLJ37842 | 213 | LTDK-ISKSAKKDRILAQ-Y-PN QKSTGIFAEFLKLIVGNQADFKKYF--NLEDK-T---PLQ--FAKDSYDEDLEN | 278 |
| KLJ72361 | 213 | LTDK-ISKSAKKDRILAQ-Y-PN QKSTGIFAEFLKLIVGNQADFKKYF--NLEDK-T---PLQ--FAKDSYDEDLEN | 278 |
| KLL20707 | 213 | LTDK-ISKSAKKDRILAQ-Y-PN QKSTGIFAEFLKLIVGNQADFKKYF--NLEDK-T---PLQ--FAKDSYDEDLEN | 278 |
| KLL42645 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_047207273 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_047209694 | 213 | LTDK-ISKSAKKDRILAR-Y-PN QKSTGIFAEFLKLIVGNQADFKKHF--NLEDK-T---PLQ--FAKDSYDEDLEN | 278 |
| WP_050198062 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_050201642 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_050204027 | 213 | LTDK-ISKSAKKDRILAQ-Y-PN QKSTGIFAEFLKLIVGNQADFKKHF--NLEDK-T---PLQ--FAKDSYDEDLEN | 278 |
| WP_050881965 | 213 | LTDK-ISKSAKKDRILAQ-Y-PN QKSTGIFAEFLKLIVGNQADFKKYF--NLEDK-T---PLQ--FAKDSYDEDLEN | 278 |
| WP_050886065 | 213 | LTDK-ISKSAKKDRILAQ-Y-PN QKSTGIFAEFLKLIVGNQADFKKYF--NLEDK-T---PLQ--FAKDSYDEDLEN | 278 |
| AHN30376 | 213 | LTDK-ISKSAKKDRILAQ-Y-PN QKSTGIFAEFLKLIVGNQADFKKHF--NLEDK-T---PLQ--FAKDSYDEDLEN | 278 |
| EAO78426 | 213 | LTDK-ISKSAKKDRILAQ-Y-PN QKSTGIFAEFLKLIVGNQADFKKYF--NLEDK-T---PLQ--FAKDSYDEDLEN | 278 |
| CCW42055 | 213 | LTDK-ISKSAKKDRILAQ-Y-PD QKSTGIFAEFLKLIVGNQADFKKHF--NLEDK-T---PLQ--FAKDSYDEDLEN | 278 |
| WP_003041502 | 212 | LTEK-VSKSRRLENLIAH-Y-PA EKKNTLFGNLIALFLGLQPNFKTNF--QLSED-A---KLQ--FSKDTYEEDLEG | 277 |
| WP_037593752 | 213 | LTEK-VSKSSRLENLIAH-Y-PT EKKNTLFGNLIALSLGLQPNFKINF--QLSED-A---KLQ--FSKDTYEEDLEE | 278 |
| WP_049516684 | 213 | LTEK-VSKSRRLENLVEC-Y-PT EKKNTLFGNLIALSLGLQPNFKINF--QLSED-A---KLQ--FSKDTYEEDLEG | 278 |
| GAD46167 | 212 | LTEK-VSKSSRLENLIAH-Y-PT EKKNTLFGNLIALSLGLQPNFKTNF--QLSED-A---KLQ--FSKDTYEEDLEE | 277 |
| WP_018363470 | 213 | LTEK-ISKSRRLENLINN-Y-PK EKKNTLFGNLIALALGLOPNFKTNF--KLSED-A---KLQ--FSKDTYEEDLEE | 278 |
| WP_003043819 | 212 | LSAR-LSKSKRLEKLIAV-F-PN EKKNGLFGNIIALALGLTPNFKSNF--DLTED-A---KLQ--LSKDTYDDDLDE | 277 |
| WP_006269658 | 212 | LTEK-VSKSSRLENLIAH-Y-PT EKKNTLFGNLIALSLDLHPNFKINF--QLSED-A---KLQ--FSKDTYEEDLEG | 277 |
| WP_048800889 | 212 | LTEK-VSKSRRLENLVKC-Y-PT EKKNTLFGNLIALSLGLOPNFKTNF--QLSED-A---KLQ--FSKDTYEEDLEE | 277 |
| WP_012767106 | 212 | LSAR-LSKSRRLENLIAQ-L-PG EKRNGLFGNLIALSLGLTPNFKSNF--DLAED-A---KLQ--LSKDTYDDDLDN | 277 |
| WP_014612333 | 212 | LSAR-LSKSKRLENLIAQ-L-PG EKKNGLFGNLIALSLGLTPNFKSNF--DLAED-A---KLQ--LSKDTYDDDLDN | 277 |
| WP_015017095 | 212 | LSAR-LSKSRRLENLIAQ-L-PG EKRNGLFGNLIALSLGLTPNFKSNF--DLAED-A---KLQ--LSKDTYDDDLDN | 277 |
| WP_015057649 | 212 | LSAR-LSKSRRLENLIAQ-L-PG EKRNGLFGNLIALSLGLTPNFKSNF--DLAED-A---KLQ--LSKDTYDDDLDN | 277 |
| WP_048327215 | 212 | LSAR-LSKSRRLENLIAQ-L-PG EKRNGLFGNLIALSLGLIPNFKSNF--DLAED-A---KLQ--LSKDTYDDDLDN | 277 |
| WP_049519324 | 212 | LSAR-LSKSRRLENLIAQ-L-PG EKRNGLFGNLIALSLGLTPNFKSNF--DLAED-A---KLQ--LSKDTYDDDLDN | 277 |
| WP_012515931 | 212 | LSAA-LSKSKRLENLISL-I-PG QKKTGIFGNIIALSLGLTPNFKANF--GLSKD-V---KLQ--LAKDTYADDLDS | 277 |
| WP_021320964 | 212 | LSAA-LSKSKRLENLISL-I-PG QKKTGIFGNIIALSLGLTPNFKANF--GLSKD-V---KLQ--LAKDTYADDLDS | 277 |
| WP_037581760 | 212 | LSAA-LSKSKRLENLISL-I-PG QKKTGIFGNIIALSLGLTPNFKANF--GLSKD-V---KLQ--LAKDTYADDLDS | 277 |
| WP_004232481 | 212 | LTEK-ISKSRRLENLIKQ-Y-PT EKKNTLFGNLVALALGLQPNFKTNF--KLSED-A---KLQ--FSKDTYDEDLEE | 277 |
| WP_009854540 | 213 | LTEK-ISKSRRLENLIKY-Y-PT EKKNTLFGNLIALALGLQPNFKMNF--KLSED-A---KLQ--FSKDTYEEDLEE | 278 |
| WP_012962174 | 213 | LTEK-FSKSRRLENLIKH-Y-PT EKKNTLFGNLVALALGLQPNFKTSF--KLSED-A---KLQ--FSKDTYEEDLEE | 278 |
| WP_039695303 | 213 | LTEK-ISKSRRLENLIKY-Y-PT EKKNTLFGNLIALALGLOPNFKTNF--KLSED-A---KLQ--FSKDTYEEDLEE | 278 |
| WP_014334983 | 212 | LTEK-VSKSRRLENLIKQ-Y-PT EKKNTLFGNLIALALGLQPNFKINF--KLSED-A---KLQ--FSKDIYEEDLEE | 277 |
| WP_003099269 | 212 | LTSK-TSKSRRLENLIAE-I-PN QKRNMLFGNLVSLALGLTPNFKTNF--ELLED-A---KLQ--ISKDSYEEDLDN | 277 |
| AHY15608 | 212 | LTSK-TSKSRRLENLIAE-I-PN QKRNMLFGNLVSLALGLTPNFKTNF--ELLED-A---KLQ--ISKDSYEEDLDN | 277 |
| AHY17476 | 212 | LTSK-TSKSRRLENLIAE-I-PN QKRNMLFGNLVSLALGLTPNFKTNF--ELLED-A---KLQ--ISKDSYEEDLDN | 277 |
| ESR09100 | | ----------------------- ------------------------------------------------------ | |
| AGM98575 | 212 | LTSK-TSKSRRLENLIAE-I-PN QKRNMLFGNLVSLALGLTPNFKTNF--ELLED-A---KLQ--ISKDSYEEDLDN | 277 |
| ALF27331 | 212 | LTDK-ISKSAKKDRVLKL-F-PN EKSNGRFAEFLKLIVGNQADFKKHF--ELEEK-A---PLQ--FSKDTYEEELEV | 277 |
| WP_018372492 | 210 | FTEN-SSKAKRVETILGL-F-PD ETAAGNLDKFLKLMLGNQADFKKVF--DLEEK----iTLQ--FSKDSYEEDLEL | 275 |
| WP_045618028 | 213 | FTDK-ISKSAKRERVLKL-F-PD EKSTGLFSEFLKLIVGNQADFKKHF--DLEEK-A---PLQ--FSKDTYDEDLEN | 278 |
| WP_045635197 | 212 | FTDK-ISKSAKRERVLKL-F-PD EKSTGLFSEFLKLIVGNQADFKKHF--DLEDK-A---PLQ--FSKDTYDEDLEN | 277 |
| WP_002263549 | 212 | LTDK-ISKSAKKDRVLKL-F-PN EKSNGRFAEFLKLIVGNQADFKKHF--ELEEK-A---PLQ--FSKDTYEEELEV | 277 |
| WP_002263887 | 212 | LTDK-ISKSAKKDRVLKL-F-PN EKSNGRFAEFLKLIVGNQADFKKHF--ELEEK-A---PLQ--FSKDTYEEELEV | 277 |
| WP_002264920 | 212 | LTEK-ISKSRRLEKLINN-Y-PK EKKNTLFRNLVALSLGLQPNFKTNF--KLSED-A---KLQ--FSKDTYEEDLEE | 277 |
| WP_002269043 | 212 | LTDK-ISKSAKKDRVLKL-F-PN EKSNGRFAEFLKLIVGNQADFKKHF--ELEEK-A---PLQ--FSKDIYEEELEV | 277 |
| WP_002269448 | 212 | LTDK-ISKSAKKDRVLKL-F-PN EKSNGRFAEFLKLIVGNQADFKKHF--ELEEK-A---PLQ--FSKDTYEEELEV | 277 |
| WP_002271977 | 212 | LTDK-ISKSAKKDRVLKL-F-PN EKSNGRFAEFLKLIVGNQADFKKHF--ELEEK-A---PLQ--FSKDTYEEDLEE | 277 |
| WP_002272766 | 212 | LTDK-ISKSAKKDRVLKL-F-PN EKSNGRFAEFLKLIVGNQADFKKHF--ELEEK-A---PLQ--FSKDTYEEDLEE | 277 |
| WP_002273241 | 212 | LTDK-ISKSAKKDRVLKL-F-PN EKSNGRFAEFLKLIVGNQADFKKHF--ELEEK-A---PLQ--FSKDTYEEELEV | 277 |
| WP_002275430 | 212 | LTDK-ISKSAKKDRVLKL-F-PN EKSNGRFAEFLKLIVGNQADFKKHF--ELEEK-A---PLQ--FSKDTYEEELEV | 277 |
| WP_002276448 | 212 | LTDK-ISKSAKKDRVLKL-F-PN EKSNGRFAEFLKLIVGNQADFKKHF--ELEEK-A---PLQ--FSKDTYEEELEV | 277 |
| WP_002277050 | 212 | LTEK-ISKSRRLEKLINN-Y-PK EKKNTLFGNLIALSLGLQPNFKINF--KLSED-A---KLQ--FSKDTYEEDLEE | 277 |
| WP_002277364 | 212 | LTDK-ISKSAKKDRVLKL-F-PN EKSNGRFAEFLKLIVGNQADFKKHF--ELEEK-A---PLQ--FSKDTYEEELEV | 277 |
| WP_002279025 | 212 | LTDK-ISKSAKKDRVLKL-F-PN EKSNGRFAEFLKLIVGNQADFKKHF--ELEEK-A---PLQ--FSKDTYEEELEV | 277 |
| WP_002279859 | 212 | LTEK-ISKSRRLEKLINN-Y-PK EKKNTLFGNLIALSLGLQPNFKTNF--KLSED-A---KLQ--FSKDTYEEELEV | 277 |
| WP_002280230 | 212 | LTDK-ISKSAKKDRVLKL-F-PN EKSNGRFAEFLKLIVGNQADFKKHF--ELEEK-A---PLQ--FSKDTYEEELEV | 277 |
| WP_002281696 | 212 | LTDK-ISKSAKKDRVLKL-F-PN EKSNGRFAEFLKLIVGNQADFKKHF--ELEEK-A---PLQ--FSKDTYEEELEV | 277 |
| WP_002282247 | 212 | LTEK-ISKSRRLEKLINN-Y-PK EKKNTLFGNLIALSLGLQPNFKTNF--KLSED-A---KLQ--FSKDTYEEDLEE | 277 |
| WP_002282906 | 212 | LTDK-ISKSAKKDRVLKL-F-PN EKSNGRFAEFLKLIVGNQADFKKHF--ELEEK-A---PLQ--FSKDTYEEELEV | 277 |
| WP_002283846 | 212 | LTDK-ISKSAKKDRVLKL-F-PN EKSNGRFAEFLKLIVGNQADFKKHF--ELEEK-A---PLQ--FSKDIYEEELEV | 277 |
| WP_002287255 | 212 | LTDK-ISKSAKKDRVLKL-F-PN EKSNGRFAEFLKLIVGNQADFKKHF--ELEEK-A---PLQ--FSKDTYEEELEV | 277 |
| WP_002288990 | 212 | LTDK-ISKSAKKDRVLKL-F-PN EKSNGRFAEFLKLIVGNQADFKKHF--ELEEK-A---PLQ--FSKDIYEEELEV | 277 |
| WP_002289641 | 212 | LTDK-ISKSAKKDRVLKL-F-PN EKSNGCFAEFLKLIVGNQADFKKHF--ELEEK-A---PLQ--FSKDTYEEELEV | 277 |
| WP_002290427 | 212 | LTDK-ISKSAKKDRVLKL-F-PN EKSNGRFAEFLKLIVGNQADFKKHF--ELEEK-A---PLQ--FSKDIYEEELEV | 277 |
| WP_002295753 | 212 | LTDK-ISKSAKKDRVLKL-F-PN EKSNGRFAEFLKLIVGNQADFKKHF--ELEEK-A---PLQ--FSKDTYEEELEV | 277 |
| WP_002296423 | 212 | LTDK-ISKSAKKDRVLKL-F-PN EKSNGRFAEFLKLIVGNQADFKKHF--ELEEK-A---PLQ--FSKDIYEEELEV | 277 |
| WP_002304487 | 212 | LTEK-VSKSRRLENLVEC-Y-PT EKKNTLFGNLIALSLGLQPNFKTNF--QLSED-A---KLQ--FSKDTYEEDLEG | 277 |
| WP_002305844 | 212 | LTDK-ISKSAKKDRVLKL-F-PN EKSNGRFAEFLKLIVGNQADFKKHF--ELEEK-A---PLQ--FSKDTYEEELEV | 277 |
| WP_002307203 | 212 | LTDK-ISKSAKKDRVLKL-F-PN EKSNGRFAEFLKLIVGNQADFKKHF--ELEEK-A---PLQ--LSKDTYEEELEV | 277 |
| WP_002310390 | 212 | LTDK-ISKSAKKDRVLKL-F-PN EKSNGRFAEFLKLIVGNQADFKKHF--ELEEK-A---PLQ--FSKDIYEEELEV | 277 |
| WP_002352408 | 212 | LTDK-ISKSAKKDRVLKL-F-PN EKSNGRFAEFLKLIVGNQADFKKHF--ELEEK-A---PLQ--FSKDIYEEELEV | 277 |
| WP_012997688 | 212 | LTDK-ISKSAKKDRVLKL-F-PN EKSNGRFAEFLKLIVGNQADFKKHF--ELEEK-A---PLQ--FSKDTYEEELEV | 277 |
| WP_014677909 | 212 | LTDK-ISKSAKKDRVLKL-F-PN EKSNGRFAEFLKLIVGNQADFKKHF--ELEEK-A---PLQ--FSKDIYEEELEV | 277 |
| WP_019312892 | 212 | LTDK-ISKSAKKDRVLKL-F-PN EKSNGRFAEFLKLIVGNQADFKKHF--ELEEK-A---PLQ--FSKDTYEEELEV | 277 |
| WP_019313659 | 212 | LTDK-ISKSAKKDRVLKL-F-PN EKSNGRFAEFLKLIIGNQADFKKHF--ELEEK-A---PLQ--FSKDTYEEELEV | 277 |
| WP_019314093 | 212 | LTDK-ISKSAKKDRVLKL-F-PN EKSNGRFAEFLKLIVGNQADFKKHF--ELEEK-A---PLQ--LSKDTYEEELEV | 277 |
| WP_019315370 | 212 | LTDK-ISKSAKKDRVLKL-F-PN EKSNGRFAEFLKLIVGNQADFKKHF--ELEEK-A---PLQ--FSKDTYEEELEV | 277 |
| WP_019803776 | 212 | LTDK-ISKSAKKDRVLKL-F-PN EKSNGRFAEFLKLIVGNQADFKKHF--ELEEK-V---PLQ--FSKDTYEEELEV | 277 |
| WP_019805234 | 212 | LTDK-ISKSAKKDRVLKL-F-PN EKSNGRFAEFLKLIVGNQADFKKHF--ELEEK-A---PLQ--FSKDIYEEELEV | 277 |
| WP_024783594 | 212 | LTDK-ISKSAKKDRVLKL-F-PN EKSNGRFAEFLKLIVGNQADFKKHF--ELEEK-A---PLQ--FSKDTYEEELEV | 277 |
| WP_024784288 | 212 | LTEK-ISKSRRLEKLINN-Y-PK EKKNTLFGNLIALSLGLQPNFKTNF--KLSED-A---KLQ--FSKDTYEEDLEE | 277 |
| WP_024784666 | 212 | LTDK-ISKSAKKDRVLKL-F-PN EKSNGRFAEFLKLIVGNQADFKKHF--ELEEK-A---PLQ--FSKDTYEEELEV | 277 |
| WP_024784894 | 212 | LTDK-ISKSAKKDRVLKL-F-PN EKSNGRFAEFLKLIVGNQADFKKHF--ELEEK-A---PLQ--FSKDTYEEELEV | 277 |
| WP_024786433 | 212 | LTEK-ISKSRRLEKLINN-Y-PK EKKNTLFGNLIALSLGLQPNFKTNF--KLSED-A---KLQ--FSKDTYEEDLEE | 277 |
| WP_049473442 | 212 | LTDK-ISKSAKKDRVLKL-F-PN EKSNGRFAEFLKLIVGNQADFKKHF--ELEEK-A---PLQ--FSKDTYEEDLEE | 277 |
| WP_049474547 | 212 | LTDK-ISKSAKKDRVLKL-F-PN EKSNGRFAEFLKLIVGNQADFKKHF--ELEEK-A---PLQ--FSKDTYEEELEV | 277 |
| EMC03581 | 205 | LTDK-ISKSAKKDRVLKL-F-PN EKSNGRFAEFLKLIVGNQADFKKHF--ELEEK-A---PLQ--FSKDTYEEELEV | 270 |
| WP_000428612 | 213 | FTDK-ISKSAKRERVLKL-F-PD EKSTGLFSEFLKLIVGNQADFKKHF--DLEEK-A---PLQ--FSRDTYDEDLEN | 278 |
| WP_000428613 | 213 | FTDK-ISKSAKRERVLKL-F-PD EKSTGLFSEFLKLIVGNQADFKKHF--DLGEK-A---PLQ--FSKDTYDEDLEN | 278 |
| WP_049523028 | 212 | FTDK-ISKSAKRDRVLKL-F-PD EKSTGLFSEFLKLIVGNQADFKKHF--DLEEK-A---PLQ--FSKDTYEEDLES | 277 |
| WP_003107102 | 181 | LTEK-MSKSRRLENLIAK-I-PN QKKNTLFGNLISLSLGLTPNFKANF--ELSED-A---KLQ--ISKESFEEDLDN | 246 |
| WP_054279288 | 214 | LTEK-ISKTRRLENLISN-I-PG QKKNGLFGNLIALSLGLTPNFKSHF--NLPED-A---KLQ--LAKDTYDEELNN | 279 |
| WP_049531101 | 213 | FTDK-ISKSTKRERVLKL-F-PD QKSTGLFSEFLKLIVGNQADFKKHF--DLEEK-A---PLQ--FSKDTYDEDLEN | 278 |
| WP_049538452 | 213 | FSDK-ISKSAKRERVLKL-F-PD EKSTGLFSEFLKLIVGNQADFKKHF--DLEEK-A---PLQ--FSKDTYDEDLEN | 278 |
| WP_049549711 | 213 | FTDK-ISKSAKRERVLKL-F-PD EKSTGLFSEFLKLIVGNQADFKKHF--DLGEK-A---PLQ--FSKDTYDEDLEN | 278 |
| WP_007896501 | 214 | LTAK-TSKSKRLESLISE-F-PG QKKNGLFGNLLALALGLRPNFKSNF--GLSED-A---KLQ--ITKDTYEEELDN | 279 |
| EFR44625 | 166 | LTAK-TSKSKRLESLISE-F-PG QKKNGLFGNLLALALGLRPNFKSNF--GLSED-A---KLQ--ITKDTYEEELDN | 231 |
| WP_002897477 | 212 | FTDK-ISKSAKRERVLKL-F-PD EKSTGLFSEFLKLIVGNQADFKKHF--DLEEK-A---PLQ--FSKDTYDEELEN | 277 |
| WP_002906454 | 212 | FTDK-ISKSTKRERVLKL-F-SD EKSTGLFSEFLKLIVGNQADFKKHF--DLEEK-A---PLQ--FSKDTYDEDLEN | 277 |
| WP_009729476 | 213 | FTDK-ISKSAKRERVLKL-F-PD EKSTGLFSEFLKLIVGNQADFKKHF--DLEEK-A---PLQ--FSRDTYDEDLEN | 278 |
| CQR24647 | 212 | ITAK-ISKSRKVEAVLEQ-F-PD QKKNSFFGNMVSLVFGLMPNFKSNF--ELDED-A---KLQ--FSRDSYDEDLEN | 277 |
| WP_000066813 | 213 | FTDK-ISKSTKRERVLKL-F-PD EKSTGLFSEFLKLIVGNQADFKKHF--DLEEK-A---PLQ--FSKDTYDEDLEN | 278 |
| WP_009754323 | 213 | FTGK-ISKSVKREHVLKL-F-PD EKSTGLFSEFLKLIVGNQADFKKHF--DLEEK-A---SLQ--FSKDTYDEDLEN | 278 |
| WP_044674937 | 212 | LVEK-VSKSRRLENILHY-F-PN EKKNGLFGNFLALALGLQPNFKTNF--ELAED-A---KIQ--FSKETYEEDLEE | 277 |
| WP_044676715 | 212 | LVEK-VSKSRRLENILHY-F-PN EKKNGLFGNFLTLALGLQPNFKTNF--ELAED-A---KIQ--FSKETYEEDLEE | 277 |
| WP_044680361 | 212 | LVEK-VSKSRRLENILHY-F-PN EKKNGLFGNFLALALGLQPNFKINF--ELAED-A---KIQ--FSKETYEEDLEE | 277 |
| WP_044681799 | 212 | LVEK-VSKSRRLENILHY-F-PN EKKNGLFGNFLALALGLQPNFKTNF--ELAED-A---KIQ--FSKETYEEDLEE | 277 |
| WP_049533112 | 212 | LTEK-VSKSRRLENLIAH-Y-PA EKKNTLFGNLIALSLGLQPNFKINF--QLSED-A---KLQ--FSKDTYEEDLEG | 277 |
| WP_029090905 | 172 | LLDRmMNRSSKVKFLIEL---TG KQDKPLLKELFNLIVGLKAKPASIFe---QEN1AtivETM-nMSTEQVOLDLLT | 243 |
| WP_006506696 | 211 | LKKP-LSKKAKVDEVMTL-IaPE KDYKSAFKELVTGIAGNKMNVTKMIIcEPIKQ-Gds-EIK1kFSDSNYDDQFSE | 283 |
| AIT42264 | 212 | LSAR-LSKSRRLENLIAQ-L-PG EKKNGLFGNLIALSLGLTPNFKSNF--DLAED-A---KLQ--LSKDTYDDDLDN | 277 |
| WP_034440723 | 218 | FKQD-ISRSKKLDQAIAL-F-QG -KRQSLFGIFLTLIVGNKANFQKIF--NLEDD----IKLD--LKEEDYDENLEE | 283 |
| AKQ21048 | 212 | LSAR-LSKSRRLENLIAQ-L-PG EKKNGLFGNLIALSLGLTPNFKSNF--DLAED-A---KLQ--LSKDTYDDDLDN | 277 |
| WP_004636532 | 211 | LSSK-QSRSRKHEQIMAL-F-PN ENKLGNFGRFMMLIVGNTSNFKPVF--DLDDE-Y---KLK--LSDETYEEDLDT | 276 |
| WP_002364836 | 218 | LTEK-ASRTKKSEKVLQQ-F-PQ EKANGLFGQFLKLMVGNKADFKKVF--GLEEE-A---KI--tYASESYEEDLEG | 283 |
| WP_016631044 | 169 | LTEK-ASRTKKSEKVLQQ-F-PQ EKANGLFGQFLKLMVGNKADFKKVF--GLEEE-A---KI--tYASESYEEDLEG | 234 |
| EMS75795 | 1 | ----------------------- ----------------------------MDEE-A---KIQ--LSKESYEEELES | 20 |
| WP_002373311 | 218 | LTEK-ASRTKKSEKVLQQ-F-PQ EKANGLFGQFLKLMVGNKADFKKVF--GLEEE-A---KI--tYASESYEEDLEG | 283 |
| WP_002378009 | 218 | LTEK-ASRTKKSEKVLQQ-F-PQ EKANGLFGQFLKLMVGNKADFKKVF--GLEEE-A---KI--tYASESYEEDLEG | 283 |
| WP_002407324 | 218 | LTEK-ASRTKKSEKVLQQ-F-PQ EKANGLFGQFLKLMVGNKADFKKVF--GLEEE-A---KI--tYASESYEEDLEG | 283 |
| WP_002413717 | 218 | LTEK-ASRTKKSEKVLQQ-F-PQ EKANGLFGQFLKLMVGNKADFKKVF--GLEEE-A---KI--tYASESYEEDLEG | 283 |
| WP_010775580 | 218 | LTEK-ASRTKKSEKVLQQ-F-PQ EKANGLFGQFLKLMVGNKADFKKVF--GLEEE-A---KIKitYASESYEEDLEG | 285 |
| WP_010818269 | 218 | LTEK-ASRTKKSEKVLQQ-F-PQ EKANGLFGQFLKLMVGNKADFKKVF--GLEEE-A---KI--tYASESYEEDLEG | 283 |
| WP_010824395 | 218 | LTEK-ASRTKKSEKVLQQ-F-PQ EKANGLFGQFLKLMVGNKADFKKVF--GLEEE-A---KI--tYASESYEEDLEG | 283 |
| WP_016622645 | 218 | LTEK-ASRTKKSEKVLQQ-F-PQ EKANGLFGQFLKLMVGNKADFKKVF--GLEEE-A---KI--tYASESYEEDLEG | 283 |
| WP_033624816 | 218 | LTEK-ASRTKKSEKVLQQ-F-PQ EKANGLFGQFLKLMVGNKADFKKVF--GLEEE-A---KI--tYASESYEEDLEG | 283 |
| WP_033625576 | 218 | LTEK-ASRTKKSEKVLQQ-F-PQ EKANGLFGQFLKLMVGNKADFKKVF--GLEEE-A---KI--tYASESYEEDLEG | 283 |
| WP_033789179 | 218 | LTEK-ASRTKKSEKVLQQ-F-PQ EKANGLFGQFLKLMVGNKADFKKVF--GLEEE-A---KI--tYASESYEEDLEG | 283 |
| WP_002310644 | 217 | FTEK-MSKTKKAETLLKY-F-PH EKSNGYLSQFIKLMVGNQGNFKNVF--GL-EE-A---KLQ--FSKETYEEDLEE | 281 |
| WP_002312694 | 217 | FTEK-MSKTKKAETLLKY-F-PH EKSNGYLSQFIKLMVGNQGNFKNVF--GL-EEeA---KLQ--FSKETYEEDLEE | 282 |
| WP_002314015 | 217 | FTEK-MSKTKKAETLLKY-F-PH EKSNGYLSQFIKLMVGNQGNFKNVF--GL-EEeA---KLQ--FSKETYEEDLEE | 282 |
| WP_002320716 | 217 | FTEK-MSKTKKAETLLKY-F-PH EKSNGYLSQFIKLMVGNQGNFKNVF--GL-EEeA---KLQ--FSKETYEEDLEE | 282 |
| WP_002330729 | 217 | FTEK-MSKTKKAETLLKY-F-PH EKSNGYLSQFIKLMVGNQGNFKNVF--GL-EE-A---KLQ--FSKETYEEDLEE | 281 |
| WP_002335161 | 217 | FTEK-MSKTKKAETLLKY-F-PH EKSNGYLSQFIKLMVGNQGNFKNVF--GL-EEeA---KLQ--FSKETYEEDLEE | 282 |
| WP_002345439 | 217 | FTEK-MSKTKKAETLLKY-F-PH EKSNGYLSQFIKLMVGNQGNFKNVF--GL-EEeA---KLQ--FSKETYEEDLEE | 282 |
| WP_034867970 | 210 | LTDK-LSKTKKVEEILKY-Y-PT EKINSFFAQCLKLIVGNQANFKRIF--DLEAE-V---KLQ--FSKETYEEDLES | 275 |
| WP_047937432 | 217 | FTEK-MSKTKKAETLLKY-F-PH EKSNGYLSQFIKLMVGNQGNFKNVF--GL-EEeA---KLQ--FSKETYEEDLEE | 282 |
| WP_010720994 | 210 | LTDK-LSKTKKVEEILKY-Y-PT EKINSFFAQCLKLIVGNQANFKRIF--DLEAE-V---KLQ--FSKETYEEDLES | 275 |
| WP_010737004 | 210 | LTDK-LSKTKKVEEILKY-Y-PT EKINSFFAQCLKLIVGNQANFKRIF--DLEAE-V---KLQ--FSKETYEEDLES | 275 |
| WP_034700478 | 210 | LTDK-LSKTKKVEEILKY-Y-PT EKINSFFAQCLKLIVGNQANFKRIF--DLEAE-V---KLQ--FSKETYEEDLES | 275 |
| WP_007209003 | 215 | LTQQ-LSKSERADNVLKL-F-PD EKGTGIFAQFIKLIVGNQGNFKKVF--QLEED----qKLQ--LSTDDYEENIEN | 280 |
| WP_023519017 | 210 | LTER-LSKAKRVEKVLAY-Y-PS EKSTGNFAQFLKLMVGNQANFKKTF--DLEEE-M---KLN--FTRDCYEEDLNE | 275 |
| WP_010770040 | 213 | FSEK-VSRARKVEAILSV-Y-SE EKSTGTLAQFLKLMVGNQGRFKKTF--DLEED-G---IIQ--IPKEEYEEELET | 278 |
| WP_048604708 | 209 | FADK-VSRAKKAEGVLAL-F-PD EKRNGTFDQFLKMIVGNQGNFKKTF--ELEED-A---KLQ--FSKEEYDESLEA | 274 |
| WP_010750235 | 210 | LTDK-LSKSKKVEKILQY-Y-PK EKTTGCLAQFLKLIVGNQGNFKQAF--HLDEE-V---KIQ--ISKETYEEDLEK | 275 |
| AII16583 | 251 | LSAR-LSKSRRLENLIAQ-L-PG EKKNGLFGNLIALSLGLTPNFKSNF--DLAED-A---KLQ--LSKDTYDDDLDN | 316 |
| WP_029073316 | 216 | LKEP-LSKKHKADKAFAL-FdTT KDNKAAYKELCAALAGNKFNVTKMLkeAELHD-EdekDISfkFSDATEDDAFVE | 289 |
| WP_031589969 | 216 | LKEP-LSKKHKAEKAFAL-FdTT KDNKAAYKELCAALAGNKFNVTKMLkeAELHD-EdekDISfkFSDATFDDAFVE | 289 |
| KDA45870 | 210 | FKDNtFSKTKKSEELLKL---SG -KKNQLAHQLFKMMVGNMGSFKKVL--GTDEE----hKLS--FGKDTYEDDLND | 275 |
| WP_039099354 | 207 | LLDNhQSASNRORQALLLiYtPS KONKAIATELLKAILGLKAKFNVLT--GIEAEdVktwTLT--FNAENFDEEMVK | 285 |
| AKP02966 | 209 | LIGR-GNATQKSSNILNN-F--T KETKKLLKEVINLILGNVAHLNTIFktSLTKDeE---KLS--FSGKDIESKLDD | 278 |
| WP_010991369 | 218 | LVEK-VTRKEKLERILKL-Y-PG EKSAGMFAQFISLIVGSKGNFQKPF--DLIEK-S---DIE--CAKDSYEEDLES | 283 |
| WP_033838504 | 218 | LVEK-VTRKEKLERILKL-Y-PG EKSAGMFAQFISLIVGSKGNFQKPF--DLIEK-S---DIE--CAKDSYEEDLES | 283 |
| EHN60060 | 221 | LVEK-VTRKEKLERILKL-Y-PG EKSAGMFAQFISLIVGSKGNFQKPF--DLIEK-S---DIE--CAKDSYEEDLES | 286 |
| EFR89594 | 1 | ---------------LKL-Y-PG EKSTGMFAQFISLIVGSKGNFQKPF--DLIEK-S---DIE--CAKDSYEEDLES | 52 |
| WP_038409211 | 218 | LSEK-LTRREKLDKILKL-Y-TG EKSTGMFARFINLIIGSKGDFKKVF--DLDEK-A---EIE--CAKDTYEEDLEA | 283 |
| EFR95520 | | ----------------------- ------------------------------------------------------ | |
| WP_003723650 | 218 | LAGK-FTRREKFERILQL-Y-PG EKSTGMFAQFISLIVGSKGNFQKVF--DLIEK-T---DIE--CAKDSYEEDLET | 283 |
| WP_003727705 | 218 | LAGK-FTRREKFERILRL-Y-PG EKSTGMFAQFISLIVGNKGNFQKVF--NLVEK-T---DIE--CAKDSYEEDLEA | 283 |
| WP_003730785 | 218 | LAGK-FTRREKFERILRL-Y-PG EKSTGMFAQFISLIVGNKGNFQKVF--NLVEK-T---DIE--CAKDSYEEDLEA | 283 |
| WP_003733029 | 218 | LAEK-FTRKDKLDKILSL-Y-PG EKTTGVFAQFVNIIVGSTGKFKKHF--NLHEK-K---DIN--CAEDTYDTDLES | 283 |
| WP_003739838 | 218 | LAGK-FTRKEKLERILQL-Y-PG EKSTGMFAQFISLIVGSKGNFQKVF--DLVEK-T---DIE--CAKDSYEEDLEA | 283 |
| WP_014601172 | 218 | LAGK-FTRREKFERILQL-Y-PG EKSTGMFAQFISLIVGSKGNFQKVF--DLIEK-T---DIE--CAKDSYEEDLEA | 283 |
| WP_023548323 | 218 | LAGK-FTRREKFERILQL-Y-PG EKSTGMFAQFISLIVGSKGNFQKVF--DLIEK-T---DIE--CAKDSYEEDLET | 283 |
| WP_031665337 | 218 | LAGK-FTRREKFERILQL-Y-PG EKSTGMFAQFISLIVGSKGNFQKVF--DLIEK-T---DIE--CAKDSYEEDLET | 283 |
| WP_031669209 | 218 | LAEK-FTRKDKLDKILSL-Y-PG EKTTGVFAQFVNIIVGSTGKFKKHF--NLHEK-K---DIN--CAEDTYDTDLES | 283 |
| WP_033920898 | 218 | LARK-FTRREKFERILQL-Y-PG EKSTGMFAQFISLIVGSKGNFQKVF--DLIEK-T---DIE--CAKDSYEEDLET | 283 |
| AKI42028 | 221 | LAGK-FTRREKFERILQL-Y-PG EKSTGMFAQFISLIVGSKGNFQKVF--DLIEK-T---DIE--CAKDSYEEDLEA | 286 |
| AKI50529 | 221 | LARK-FTRREKFERILQL-Y-PG EKSTGMFAQFISLIVGSKGNFQKVF--DLIEK-T---DIE--CAKDSYEEDLET | 286 |
| EFR83390 | | ----------------------- ------------------------------------------------------ | |
| WP_046323366 | 218 | FSEK-LTKREKLDKILNL-Y-PN EKSTDLFAQFISLIIGSKGNFKKFF--NLTEK-T---DIE--CAKDSYEEDLEV | 283 |
| AKE81011 | 228 | LSAR-LSKSRRLENLIAQ-L-PG EKKNGLFGNLIALSLGLTPNFKSNF--DLAED-A---KLQ--LSKDTYDDDLDN | 293 |
| CUO82355 | 215 | LKKP-LSKKAKVDEVMAL-IsPE KEFKSAYKELVIGIAGNKMNVTKMIICESIKQ-Gds-EIK1kFSDSNYDDQFSE | 287 |
| WP_033162887 | 216 | LSKI-YQRSKKADDLLKI-MnPT KEEKAAYKEFTKALVGLKFNISKMIlaQEVKK-Gdt-DIVleFSNANYDSTIDE | 288 |
| AGZ01981 | 245 | LSAR-LSKSRRLENLIAQ-L-PG EKKNGLFGNLIALSLGLTPNFKSNF--DLAED-A---KLQ--LSKDTYDDDLDN | 310 |
| AKA60242 | 212 | LSAR-LSKSRRLENLIAQ-L-PG EKKNGLFGNLIALSLGLTPNFKSNF--DLAED-A---KLQ--LSKDTYDDDLDN | 277 |
| AKS40380 | 212 | LSAR-LSKSRRLENLIAQ-L-PG EKKNGLFGNLIALSLGLTPNFKSNF--DLAED-A---KLQ--LSKDTYDDDLDN | 277 |
| 4UN5_B | 216 | LSAR-LSKSRRLENLIAQ-L-PG EKKNGLFGNLIALSLGLTPNFKSNF--DLAED-A---KLQ--LSKDTYDDDLDN | 281 |
| WP_010922251 | 278 | 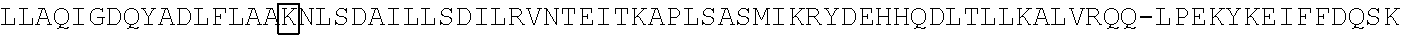 | 356 |
| WP_039695303 | 279 | LLGKIGDDYADLFTSAKNLYDAILLSGILTVDDNSTKAPLSASMIKRYVEHHEDLEKLKEFIKAN-KSELYHDIFKDKNK | 357 |
| WP_045635197 | 278 | LLGQIGDDFTDLFVSAKKLYDAILLSGILTVTDPSTKAPLSASMIERYENHONDLAALKOFIKNN-LPEKYDEVESDQSK | 356 |
| 5AXW_A | 158 | ---------------------------------------------------------LERLKKDG-------EVR----- | 168 |
| WP_009880683 | 1 | ---------------------------------------LSASMIKRYDEHHQDLTLLKALVRQQ-LPEKYKEIFFDQSK | 40 |
| WP_010922251 | 278 | LLAQIGDQYADLFLAAKNLSDAILLSDILRVNTEITKAPLSASMIKRYDEHHODLTLLKALVRQQ-LPEKYKEIFFDQSK | 356 |
| WP_011054416 | 278 | LLAQIGDQYADLFLAAKNLSDATLLSDILRVNSEITKAPLSASMIKRYDEHHQDLTLLKALVRQQ-LPEKYKEIFFDQSK | 356 |
| WP_011284745 | 278 | LLAQIGDQYADLFLAAKNLSDAILLSDILRLNSEITKAPLSASMIKRYDEHHODLTLLKALVRQQ-LPEKYKEIFFDQSK | 356 |
| WP_011285506 | 278 | LLAQIGDQYADLFLAAKNLSDAILLSDILRVNTEITKAPLSASMIKRYDEHHQDLTLLKALVROQ-LPEKYKEIFFDQSK | 356 |
| WP_011527619 | 278 | LLAQIGDQYADLFLAAKNLSDAILLSDILRLNSEITKAPLSASMIKRYDEHHQDLTLLKALVRQQ-LPEKYKEIFFDQSK | 356 |
| WP_012560673 | 278 | LLAQIGDQYADLFLAAKNLSDAILLSDILRVNTEITKAPLSASMIKRYDEHHQDLTLLKALVRQQ-LPEKYKEIFFDQSK | 356 |
| WP_014407541 | 278 | LLAQIGDQYADLFLAAKNLSDAILLSDILRVNSEITKAPLSASMIKRYDEHHQDLTLLKALVRQQ-LPEKYKEIFFDQSK | 356 |
| WP_020905136 | 278 | LLAQIGDQYADLFLAAKNLSDAILLSDILRLNSEITKAPLSASMIKRYDEHHQDLTLLKALVROQ-LPEKYKEIFFDQSK | 356 |
| WP_023080005 | 278 | LLAQIGDQYADLFLAAKNLSDAILLSDILRVNSEITKAPLSASMIKRYDEHHQDLTLLKALVRQQ-LPEKYKEIFFDQSK | 356 |
| WP_023610282 | 278 | LLAQIGDQYADLFLAAKNLSDAILLSDILRVNSEITKAPLSASMIKRYDEHHQDLTLLKALVRQQ-LPEKYKEIFFDQSK | 356 |
| WP_030125963 | 278 | LLAQIGDQYADLFLAAKNLSDAILLSDILRVNTEITKASLSASMIKRYDEHHQDLTLLKALVROQ-LPEKYKEIFFDQSK | 356 |
| WP_030126706 | 278 | LLAQIGDQYADLFLAAKNLSDATLLSDILRVNSEITKAPLSASMIKRYDEHHQDLTLLKALVROQ-LPEKYKEIFFDQSK | 356 |
| WP_031488318 | 278 | LLAQIGDQYADLFLAAKNLSDAILLSDILRVNSEITKAPLSASMIKRYDEHHQDLTLLKALVRQQ-LPEKYKEIFFDQSK | 356 |
| WP_032460140 | 278 | LLAQIGDQYADLFLAAKNLSDAILLSDILRVNTEITKAPLSASMIKRYDEHHQDLTLLKALVROQ-LPEKYKEIFFDQSK | 356 |
| WP_032461047 | 278 | LLAQIGDQYADLFLAAKNLSDAILLSDILRVNTEITKAPLSASMIKRYDEHHQDLTLLKALVROQ-LPEKYKEIFFDQSK | 356 |
| WP_032462016 | 278 | LLAQIGDQYADLFLAAKNLSDAILLSDILRVNTEITKAPLSASMIKRYDEHHQDLTLLKALVRQQ-LPEKYKEIFFDQSK | 356 |
| WP_032462936 | 278 | LLAQIGDQYADLFLAAKNLSDAILLSDILRVNSEITKAPLSASMIKRYDEHHQDLTLLKALVRQQ-LPEKYKEIFFDQSK | 356 |
| WP_032464890 | 278 | LLAQIGDQYADLFLAAKNLSDAILLSDILRLNSEITKAPLSASMIKRYDEHHQDLTLLKALVRQQ-LPEKYKEIFFDQSK | 356 |
| WP_033888930 | 103 | LLAQIGDQYADLFLAAKNLSDAILLSDILRVNTEITKAPLSASMIKRYDEHHQDLILLKALVRQQ-LPEKYKEIFFDQSK | 181 |
| WP_038431314 | 278 | LLAQIGDQYADLFLAAKNLSDATLLSDILRVNSEITKAPLSASMIKRYDEHHODLTLLKALVRQQ-LPEKYKEIFFDQSK | 356 |
| WP_038432938 | 278 | LLAQIGDQYADLFLAAKNLSDAILLSDILRVNSEITKAPLSASMIKRYDEHHQDLILLKALVRQQ-LPEKYKEIFFDQSK | 356 |
| WP_038434062 | 278 | LLAQIGDQYADLFLAAKNLSDAILLSDILRVNSEITKAPLSASMIKRYDEHHQDLTLLKALVRQQ-LPEKYKEIFFDQSK | 356 |
| BAQ51233 | 189 | LLAQIGDQYADLFLAAKNLSDAILLSDILRVNTEITKAPLSASMIKRYDEHHQDLTLLKALVRQQ-LPEKYKEIFFDQSK | 267 |
| KGE60162 | | -------------------------------------------------------------------------------- | |
| KGE60856 | | -------------------------------------------------------------------------------- | |
| WP_002989955 | 278 | LLAQIGDQYADLFLAAKNLSDAILLSDILRLNSEITKAPLSASMIKRYDEHHQDLTLLKALVRQQ-LPEKYKEIFFDQSK | 356 |
| WP_003030002 | 278 | LLGEIGDEYADLFASAKNLYDAILLSGILTVDDNSTKAPLSASMVKRYEEHQKDLKKLKDFIKVN-APDQYNAIFKDKNK | 356 |
| WP_003065552 | 281 | LLGKIGDDYADLFTSAKNLYDAILLSGILIVDDNSTKAPLSASMIKRYVEHQEDLEKLKEFIKAN-KSELYHDIFKDKNK | 359 |
| WP_001040076 | 279 | LLGQIGDEFADLFSAAKKLYDSVLLSGILTVTDLSTKAPLSASMIQRYDEHREDLKOLKQFVKAS-LPEKYQEIVADSSK | 357 |
| WP_001040078 | 279 | LLGQIGDEFADLFSAAKKLYDSVLLSGILTVIDLSTKAPLSASMIQRYDEHREDLKOLKOFVKAS-LPEKYQEIFADSSK | 357 |
| WP_001040080 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_001040081 | 279 | LLGQIGDEFADLFSAAKKLYDSVLLSGILTVIDLSTKAPLSASMIQRYDEHREDLKOLKOFVKAS-LPEKYQEIFADSSK | 357 |
| WP_001040083 | 279 | LLGQIGDEFADLFSAAKKLYDSVLLSGILTVIDLSTKAPLSASMIQRYDEHREDLKOLKOFVKAS-LPEKYQEIFADSSK | 357 |
| WP_001040085 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_001040087 | 279 | LLGQIGDEFADLFSAAKKLYDSVLLSGILTVIDLSTKAPLSASMIQRYDEHREDLKOLKOFVKAS-LPEKYQEIFADSSK | 357 |
| WP_001040088 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_001040089 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_001040090 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_001040091 | 279 | LLRQIGDEFADLFSAAKKLYDSVLLSGILTVIDLSTKAPLSASMIQRYDEHREDLKOLKQFVKAS-LPEKYQEIFADSSK | 357 |
| WP_001040092 | 279 | LLGQIGDEFADLFSAAKKLYDSVLLSGILTVTDLSTKAPLSAYMIQRYDEHREDLKOLKQFVKAS-LPEKYQEIFADSSK | 357 |
| WP_001040094 | 279 | LLGQIGDEFADLFSAAKKLYDSVLLSGILTVTDLSTKAPLSASMIQHYDEHREDLKOLKQFVKAS-LPEKYQEIFADSSK | 357 |
| WP_001040095 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_001040096 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_001040097 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_001040098 | 279 | LLGQIGDEFADLFSAAKKLYDSVLLSGILTVTDLSTKAPLSASMIQRYDEHREDLKOLKOFVKAS-LPEKYQEIFADSSK | 357 |
| WP_001040099 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_001040100 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_001040104 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_001040105 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_001040106 | 279 | LLGQIGDEFADLFSVAKKLYDSVLLSGILTVTDLSTKAPLSASMIQRYDEHREDLKOLKQFVKAS-LPEKYQEIFTDSSK | 357 |
| WP_001040107 | 279 | LLGQIGDEFADLFSVAKKLYDSVLLSGILTVTDLSTKAPLSASMIQRYDEHREDLKOLKQFVKAS-LPEKYQEIFTDSSK | 357 |
| WP_001040108 | 279 | LLGQIGDEFADLFSVAKKLYDSVLLSGILTVTDLSTKAPLSASMIQRYDEHREDLKOLKQFVKAS-LPEKYQEIFTDSSK | 357 |
| WP_001040109 | 279 | LLGQIGDEFADLFSVAKKLYDSVLLSGILTVTDLSTKAPLSASMIQRYDEHREDLKOLKQFVKAS-LPEKYQEIFTDSSK | 357 |
| WP_001040110 | 279 | LLGQIGDEFADLFSVAKKLYDSVLLSGILTVTDLSTKAPLSASMIQRYDEHREDLKOLKQFVKAS-LPEKYQEIFTDSSK | 357 |
| WP_015058523 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_017643650 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_017647151 | 279 | LLGQIGDEFADLFSVAKKLYDSVLLSGILTVTDLSTKAPLSASMIQRYDEHREDLKOLKQFVKAS-LPEKYQEIFTDSSK | 357 |
| WP_017648376 | 279 | LLGQIGDEFADLFSVAKKLYDSVLLSGILTVTDLSTKAPLSASMIQRYDEHREDLKOLKQFVKAS-LPEKYQEIFTDSSK | 357 |
| WP_017649527 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_017771611 | 279 | LLGQIGDEFADLFSVAKKLYDSVLLSGILTVTALSTKAPLSASMIQRYDEHREDLKOLKQFVKAS-LPEKYQEIFTDSSK | 357 |
| WP_017771984 | 279 | LLGQIGDEFADLFSAAKKLYDSVLLSGILTVIDLSTKAPLSASMIQRYDEHREDLKOLKQFVKAS-LPEKYQEIFADSSK | 357 |
| CFQ25032 | 279 | LLGQIGDEFADLFSAAKKLYDSVLLSGILTVIDLSTKASLSDSMIQRYDEHREDLKQLKQFVKAS-LPEKYQEIFADSSK | 357 |
| CFV16040 | 279 | LLGQIGDEFADLFSAAKKLYDSVLLSGILTVIDLSTKAPLSASMIQRYDEHREDLKOLKQFVKAS-LPEKYQEIFADSSK | 357 |
| KLJ37842 | 279 | LLGQIGDEFADLFSAAKKLYDSVLLSGILTVIDLSTKAPLSASMIQRYDEHREDLKOLKQFVKAS-LPEKYQEIFADSSK | 357 |
| KLJ72361 | 279 | LLGQIGDEFADLFSAAKKLYDSVLLSGILTVIDLSTKAPLSASMIQRYDEHREDLKOLKQFVKAS-LPEKYQEIFADSSK | 357 |
| KLL20707 | 279 | LLGQIGDEFADLFSAAKKLYDSVLLSGILTVIDLSTKAPLSASMIQRYDEHREDLKOLKQFVKAS-LPEKYQEIFADSSK | 357 |
| KLL42645 | 279 | LLGQIGDEFADLFSVAKKLYDSVLLSGILTVTDLSTKAPLSASMIQRYDEHREDLKQLKQFVKAS-LPEKYQEIFTDSSK | 357 |
| WP_047207273 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_047209694 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_050198062 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_050201642 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_050204027 | 279 | LLGQIGDEFADLFSVAKKLYDSVLLSGILTVTDLSTKAPLSASMIQRYDEHREDLKOLKQFVKAS-LPEKYQEIFTDSSK | 357 |
| WP_050881965 | 279 | LLGQIGDEFADLFSAAKKLYDSVLLSGILTVIDLSTKAPLSASMIQRYDEHREDLKOLKQFVKAS-LPEKYQEIFADSSK | 357 |
| WP_050886065 | 279 | LLGQIGDEFADLFSAAKKLYDSVLLSGILTVIDLSTKAPLSASMIQRYDEHREDLKOLKOFVKAS-LPEKYQEIFADSSK | 357 |
| AHN30376 | 279 | LLGQIGDEFADLFSAAKKLYDSVLLSGILTVTDLSTKAPLSASMIQRYDEHREDLKOLKQFVKAS-LPEKYQEIFADSSK | 357 |
| EAO78426 | 279 | LLGQIGDEFADLFSAAKKLYDSVLLSGILTVIDLSTKAPLSASMIQRYDEHREDLKOLKQFVKAS-LPEKYQEIFADSSK | 357 |
| CCW42055 | 279 | LLGQIGDEFADLFSAAKKLYDSVLLSGILTVTDLSTKAPLSASMIQRYDEHREDLKOLKQFVKAS-LPEKYQEIFADSSK | 357 |
| WP_003041502 | 278 | LLGEVGDEYADLFASAKNLYDAILLSGILTVDDNSTKAPLSASMVKRYEEHQKDLKKFEDFIKVN-ALDQYNAIFKDKNK | 356 |
| WP_037593752 | 279 | LLGEIGDEYADLFASAKNLYDAILLSGILAVDDNTTKAPLSASMVKRYEEHQKDLKKLKDFIKVN-APDQYNAIFKDKNK | 357 |
| WP_049516684 | 279 | LLGEIGDEYADLFASAKNLYDAILLSGILAVDDNTTKAPLSASMVKRYEEHQKDLKKLKDFIKVN-APAQYDDIFKDETK | 357 |
| GAD46167 | 278 | LLGEIGDEYADLFASAKNLYDAILLSGILAVDDNTTKAPLSASMVKRYEEHQKDLKKLKDFIKVN-APDQYNAIFKDKNK | 356 |
| WP_018363470 | 279 | LLGKIGDDYADLFTSSKNLYDAILLSGILTVDDNSTKAPLSASMIKRYVEHHEDLEKLKEFIKAN-KSELYHDIFKDKTQ | 357 |
| WP_003043819 | 278 | LLGQIGDQYADLFSAAKNLSDAILLSDILRSNSEVTKAPLSASMVKRYDEHHQDLALLKTLVRQQ-FPEKYAEIFKDDTK | 356 |
| WP_006269658 | 278 | FLGEVGDEYADLFASAKNLYDAILLSGILTVDDNSTKAPLSASMVKRYEEHQKDLKKLKDFIKVN-APDQYNAIFKDKNK | 356 |
| WP_048800889 | 278 | LLGKIGDDYADLFTSAKNLYDTILLSGILAVDDNSTKALLSASMIKRYEEHQKDLKKLKDFIKVN-APAQYDDIFKDETK | 356 |
| WP_012767106 | 278 | LLAQIGDQYADLFLAAKNLSDAILLSDILRVNSEITKAPLSASMIKRYDEHHODLTLLKALVROQ-LPEKYKEIFFDQSK | 356 |
| WP_014612333 | 278 | LLAQIGNQYADLFLAAKNLSDAILLSDILRVNSEITKAPLSASMIKRYDEHHQDLTLLKALVRQQ-LPEKYKEIFFDQSK | 356 |
| WP_015017095 | 278 | LLAQIGDQYADLFLAAKNLSDAILLSDILRVNSEITKAPLSASMIKRYDEHHODLTLLKALVROQ-LPEKYKEIFFDQSK | 356 |
| WP_015057649 | 278 | LLAQIGDQYADLFLAAKNLSDAILLSDILRVNSEITKAPLSASMIKRYDEHHODLTLLKALVRQQ-LPEKYKEIFFDQSK | 356 |
| WP_048327215 | 278 | LLAQIGDQYADLFLAAKNLSDAILLSDILRVNSEITKAPLSASMIKRYDEHHQDLTLLKALVRQQ-LPEKYKEIFFDQSK | 356 |
| WP_049519324 | 278 | LLAQIGDQYADLFLAAKNLSDAILLSDILRVNSEITKAPLSASMIKRYDEHHODLTLLKALVRQQ-LPEKYKEIFFDQSK | 356 |
| WP_012515931 | 278 | LLAQIGDQYADLFLAAKNLSDAILLSDILTESDEITRAPLSASMVKRYREHHKDLVTLKTLIKDQ-LPEKYQEIFLDKTK | 356 |
| WP_021320964 | 278 | LLAQIGDQYADLFLAAKNLSDAILLSDILTESDEITRAPLSASMVKRYREHHKDLVTLKTLIKDQ-LPEKYQEIFLDKTK | 356 |
| WP_037581760 | 278 | LLAQIGDQYADLFLAAKNLSDAILLSDILTESDEITRAPLSASMVKRYREHHKDLVTLKTLIKDQ-LPEKYQEIFLDKTK | 356 |
| WP_004232481 | 278 | LLGKIGDDYADLFTAAKNLYDAILLSGILTVDDNSTKAPLSASMIKRYEEHHEDLEKLKTFIKVN-NFDKYHEIFKDKSK | 356 |
| WP_009854540 | 279 | LLGKIGDDYADLFTSAKNLYDAILLSGILTVDDNSTKAPLSASMIKRYVEHHEDLEKLKEFIKAN-KSELYHDIFKDKNK | 357 |
| WP_012962174 | 279 | LIGKIGDEYADLFTSAKNLYDAILLSGILTVADNTTKAPLSASMIKRYNEHQVDLKKLKEFIKNN-ASDKYDEIFNDKDK | 357 |
| WP_039695303 | 279 | LLGKIGDDYADLFTSAKNLYDAILLSGILTVDDNSTKAPLSASMIKRYVEHHEDLEKLKEFIKAN-KSELYHDIFKDKNK | 357 |
| WP_014334983 | 278 | LLGKVGDDYADLFISAKNLYDAILLSGILTVDDNSTKAPLSASMIKRYVEHHEDLEKLKEFIKIN-KLKLYHDIFKDKTK | 356 |
| WP_003099269 | 278 | LLAQIGDQYADLFIAAKKLSDAILLSDIITVKGASTKAPLSASMVORYEEHQQDLALLKNLVKKQ-IPEKYKEIFDNKEK | 356 |
| AHY15608 | 278 | LLAQIGDQYADLFIAAKKLSDAILLSDIITVKGASTKAPLSASMVQRYEEHQQDLALLKNLVKKQ-IPEKYKEIFDNKEK | 356 |
| AHY17476 | 278 | LLAQIGDQYADLFIAAKKLSDAILLSDIITVKGASTKAPLSASMVQRYEEHQQDLALLKNLVKKQ-IPEKYKEIFDNKEK | 356 |
| ESR09100 | | -------------------------------------------------------------------------------- | |
| AGM98575 | 278 | LLAQIGDQYADLFIAAKKLSDAILLSDIITVKGASTKAPLSASMVQRYEEHQQDLALLKNLVKKQ-IPEKYKEIFDNKEK | 356 |
| ALF27331 | 278 | LLAQIEDNYAELFLSAKKLYDSILLSGILTVTDVSTKAPLSASMIQRYNEHQMDLAQLKOFIRQK-LSDKYNEVESDVSK | 356 |
| WP_018372492 | 276 | LLSKIDEEYAALFDLAKKVYDAVLLSNILTVKEKNTKAPLSASMIKRYEEHKDDLKAFKRFFRER-LPEKYETMFKDLTK | 354 |
| WP_045618028 | 279 | LLVQIGDDFADLFLVAKKLYDAILLSGILTVTDPSTKAPLSASMIDRYENHQKDLAALKOFIKTN-LPEKYDEVFSDQSK | 357 |
| WP_045635197 | 278 | LLGQIGDDFTDLFVSAKKLYDAILLSGILTVTDPSTKAPLSASMIERYENHONDLAALKQFIKNN-LPEKYDEVFSDQSK | 356 |
| WP_002263549 | 278 | LLAQIGDNYAELFLSAKKLYDSILLSGILTVTDVGTKAPLSASMIQRYNEHOMDLAQLKOFIRQK-LSDKYNEVESDVSK | 356 |
| WP_002263887 | 278 | LLAQIGDNYAELFLSAKKLYDSILLSGILTVTDVGTKAPLSASMIQRYNEHQMDLAQLKOFIRQK-LSDKYNEVESDVSK | 356 |
| WP_002264920 | 278 | LLGKIGDDYADLFTLAKNLYDAILLSGILTADDSSTKAPLSASMIKRYAEHHEDLEKLKEFIKAN-KPELYHDIFKDETK | 356 |
| WP_002269043 | 278 | LLAQIGDNYAELFLSAKKLYDSILLSGILTVTDVSTKAPLSASMIQRYNEHOMDLAQLKOFIRQK-LSDKYNEVESDVSK | 356 |
| WP_002269448 | 278 | LLAQIGDNYAELFLSAKKLYDSILLSGILTVTDVSTKAPLSASMIQRYNEHQMDLAQLKOFIRQK-LSDKYNEVFSDVSK | 356 |
| WP_002271977 | 278 | LLGKIGDDYADLFTLAKNLYDAILLSGILTADDSSTKAPLSASMIQRYNEHQMDLAQLKQFIRQK-LSDKYNEVFSDVSK | 356 |
| WP_002272766 | 278 | LLGKIGDDYADLFTLAKNLYDAILLSGILTADDSSTKAPLSASMIQRYNEHQMDLAQLKQFIRQK-LSDKYNEVESDVSK | 356 |
| WP_002273241 | 278 | LLAQIGDNYAELFLSAKKLYDSILLSGILTVTDVSTKAPLSASMIQRYNEHQMDLAQLKOFIRQK-LSDKYNEVESDVSK | 356 |
| WP_002275430 | 278 | LLTQIGDNYAELFLSAKKLYDSILLSGILTVTDVSTKAPLSASMIQRYNEHQMDLAQLKQFIRQK-LSDKYNEVESDVSK | 356 |
| WP_002276448 | 278 | LLAQIGDNYAELFLSAKKLYDSILLSGILTVTDVSTKAPLSASMIQRYNEHQMDLAQLKQFIRQK-LSDKYNEVESDVSK | 356 |
| WP_002277050 | 278 | LLGKIGDDYADLFTLAKNLYDAILLSGILTADDSSTKAPLSASMIKRYAEHHEDLEKLKEFIKAN-KPELYHDIFKDETK | 356 |
| WP_002277364 | 278 | LLAQIGDNYAELFLSAKKLYDSILLSGILTVTDVSTKAPLSASMIQRYNEHQMDLAQLKQFIRQK-LSDKYNEVESDVSK | 356 |
| WP_002279025 | 278 | LLAQIGDNYAELFLSAKKLYDSILLSGILTADDSSTKAPLSASMIQRYNEHOMDLAQLKQFIRQK-LSDKYNEVESDVSK | 356 |
| WP_002279859 | 278 | LLAQIGDNYAELFLSAKKLYDSILLSGILTVTDVSTKAPLSASMIQRYNEHOMDLAQLKQFIRQK-LSDKYNEVESDVSK | 356 |
| WP_002280230 | 278 | LLAQIGDNYAELFLSAKKLYDSILLSGILTVTDVSTKAPLSASMIQRYNEHOMDLAQLKOFIROK-LSDKYNEVFSDVSK | 356 |
| WP_002281696 | 278 | LLAQIGDNYAELFLSAKKLYDSILLSGILTVTDVSTKAPLSASMIQRYNEHOMDLAQLKOFIRQK-LSDKYNEVESDVSK | 356 |
| WP_002282247 | 278 | LLGKIGDDYADLFTLAKNLYDAILLSGILTADDSSTKAPLSASMIKRYAEHHEDLEKLKEFIKAN-KPELYHDIFKDETK | 356 |
| WP_002282906 | 278 | LLAQIGDNYAELFLSAKKLYDSILLSGILTVTDVGTKAPLSASMIQRYNEHQMDLAQLKQFIROK-LSDKYNEVESDVSK | 356 |
| WP_002283846 | 278 | LLAQIGDNYAELFLSAKKLYDSILLSGILTVTDVSTKAPLSASMIQRYNEHOMDLAQLKQFIRQK-LSDKYNEVESDVSK | 356 |
| WP_002287255 | 278 | LLAQIGDNYAELFLSAKKLYDSILLSGILTVTDVSTKAPLSASMIQRYNEHQMDLAQLKQFIRQK-LSDKYNEVESDVSK | 356 |
| WP_002288990 | 278 | LLAQIGDNYAELFLSAKKLYDSILLSGILTVTDVSTKAPLSASMIQRYNEHQMDLAQLKQFIRQK-LSDKYNEVESDVSK | 356 |
| WP_002289641 | 278 | LLAQIGDNYAELFLSAKKLYDSILLSGILTVTDVSTKAPLSASMIQRYNEHQMDLTOLKQFIROK-LSDKYNEVESDVSK | 356 |
| WP_002290427 | 278 | LLAQIGDNYAELFLSAKKLYDSILLSGILTVTDVSTKAPLSASMIQRYNEHOMDLAQLKOFIRQK-LSDKYNEVESDVSK | 356 |
| WP_002295753 | 278 | LLAQIGDNYAELFLSAKKLYDSILLSGILTVTDVSTKAPLSASMIQRYNEHOMDLAQLKQFIRQK-LSDKYNEVESDVSK | 356 |
| WP_002296423 | 278 | LLAQIGDNYAELFLSAKKLYDSILLSGILTVTDVSTKAPLSASMIQRYNEHOMDLAQLKQFIRQK-LSDKYNEVFSDVSK | 356 |
| WP_002304487 | 278 | LLGEIGDEYADLFASAKNLYDAILLSGILAVDDNTTKAPLSASMVKRYKEHKEELAAFKRFIKEK-LPKKYEEIFKDDTK | 356 |
| WP_002305844 | 278 | LLAQIGDNYAELFLSAKKLYDSILLSGILTVTDVSTKAPLSASMIQRYNEHQMDLAQLKQFIRQK-LSDKYNEVESDVSK | 356 |
| WP_002307203 | 278 | LLAQIGDNYAELFLSAKKLYDSILLSGILTVTDVSTKAPLSASMIQRYNEHOMDLAQLKOFIRQK-LSDKYNEVESDVSK | 356 |
| WP_002310390 | 278 | LLAQIGDNYAELFLSAKKLYDSILLSGILTVTDVSTKAPLSASMIQRYNEHQMDLAQLKOFIRQK-LSDKYNEVESDVSK | 356 |
| WP_002352408 | 278 | LLAQIGDNYAELFLSAKKLYDSILLSGILTVTDVSTKAPLSASMIQRYNEHQMDLAQLKQFIRQK-LSDKYNEVFSDVSK | 356 |
| WP_012997688 | 278 | LLAQIGDNYAELFLSAKKLYDSILLSGILTVTDVSTKAPLSASMIQRYNEHOMDLAQLKOFIRQK-LSDKYNEVFSDVSK | 356 |
| WP_014677909 | 278 | LLAQIGDNYAELFLSAKKLYDSILLSGILTVTDVSTKAPLSASMIQRYNEHQMDLAQLKQFIROK-LSDKYNEVFSDVSK | 356 |
| WP_019312892 | 278 | LLAQIGDNYAELFLSAKKLYDSILLSGILTVTDVSTKAPLSASMIQRYNEHQMDLAQLKQFIRQK-LSDKYNEVFSDVSK | 356 |
| WP_019313659 | 278 | LLAQIGDNYAELFLSAKKLYDSILLSGILTVTDVGTQAPLSASMIQRYNEHQMDLAQLKOFIRQK-LSDKYNEVESDVSK | 356 |
| WP_019314093 | 278 | LLAQIGDNYAELFLSAKKLYDSILLSGILTVTDVSTKAPLSASMIQRYNEHOMDLAQLKOFIRQK-LSDKYNEVESDVSK | 356 |
| WP_019315370 | 278 | LLAQIGDNYAELFLSAKKLYDSILLSGILTVTDVSTKAPLSASMIQRYNEHOMDLTOLKQFIRQK-LSDKYNEVESDVSK | 356 |
| WP_019803776 | 278 | LLAQIGDNYAELFLSAKKLYDSILLSGILTVTDVGTKAPLSASMIQRYNEHQMDLAQLKQFIRQK-LSDKYNEVESDVSK | 356 |
| WP_019805234 | 278 | LLAQIGDNYAELFLSAKKLYDSILLSGILTVTDVSTKAPLSASMIQRYNEHQMDLAQLKOFIRQK-LSDKYNEVESDVSK | 356 |
| WP_024783594 | 278 | LLAQIGDNYAELFLSAKKLYDSILLSGILTVTDVGTKAPLSASMIQRYNEHQMDLAQLKQFIRQK-LSDKYNEVFSDVSK | 356 |
| WP_024784288 | 278 | LLGKIGDDYADLFTLAKNLYDAILLSGILTADDSSTKAPLSASMIKRYAEHHEDLEKLKEFIKAN-KPELYHDIFKDETK | 356 |
| WP_024784666 | 278 | LLAQIGDNYAELFLSAKKLYDSILLSGILTVTDVSTKAPLSASMIQRYNEHOMDLVOLKOFIRQK-LSDKYNEVFSDVSK | 356 |
| WP_024784894 | 278 | LLAQIGDNYAELFLSAKKLYDSILLSGILTVTDVSTKAPLSASMIQRYNEHQMDLTOLKQFIRQK-LSDKYNEVESDVSK | 356 |
| WP_024786433 | 278 | LLGKIGDDYADLFTLAKNLYDAILLSGILTADDSSTKAPLSASMIKRYAEHHEDLEKLKEFIKAN-KPELYHDIFKDETK | 356 |
| WP_049473442 | 278 | LLGKIGDDYADLFTLAKNLYDAILLSGILTADDSSTKAPLSASMIQRYNEHQMDLAQLKQFIRQK-LSDKYNEVESDVSK | 356 |
| WP_049474547 | 278 | LLAQIGDNYAELFLSAKKLYDSILLSGILTVTDVSTKAPLSASMIQRYNEHQMDLTQLKOFIRQK-LSDKYNEVFSDVSK | 356 |
| EMC03581 | 271 | LLAQIGDNYAELFLSAKKLYDSILLSGILTVTDVSTKAPLSASMIQRYNEHQMDLAQLKQFIROK-LSDKYNEVESDVSK | 349 |
| WP_000428612 | 279 | LLGQIGDDFADLFVAAKKLYDAILLSGILTVTDPSTKAPLSASMIERYENHQKDLATLKQFIKTN-LPEKYDEVFSDQSK | 357 |
| WP_000428613 | 279 | LLGQIGDDFADLFLVAKKLYDAILLSGILTVTDPSTKAPLSASMIERYENHQKDLAVLKQFIKNN-LPEKYDEVFSDQSK | 357 |
| WP_049523028 | 278 | LLGQIGDVYADLFVVAKKLYDAILLAGILSVKDPGTKAPLSASMIERYDNHONDLSALKQFVRRN-LPEKYAEVFSDDSK | 356 |
| WP_003107102 | 247 | LLAQIGDQYADLFIAAKNLSDAILLSDILTVKGVNTKAPLSASMVQRFNEHQDDLKLLKKLVKVQ-LPEKYKEIFDIKDK | 325 |
| WP_054279288 | 280 | LLTQIGDEYADLFLSAKNLSDAILLSDILTVNGDGTQAPLSASLIKRYEEHRQDLALLKOMFKEQ-LPDLYRDVFTDENK | 358 |
| WP_049531101 | 279 | LLGQIGDDFADLFLVAKKLYDAILLSGILTVTDPSTKAPLSASMIERYENHQKDLAALKQFIKNN-LPEKYDEVFSDQSK | 357 |
| WP_049538452 | 279 | LLGQIGDGFADLFLVAKKLYDAILLSGILTVTDPSTKAPLSASMIERYQNHONDLASLKQFIKNN-LPEKYDEVFSDQSK | 357 |
| WP_049549711 | 279 | LLGQIGDDFADLFLVAKKLYDAILLSGILTVTDPSTKAPLSASMIERYENHQKDLTTLKQFIKNN-LPEKYDEVFSDQSK | 357 |
| WP_007896501 | 280 | LLAEIGDHYADLFLAAKNLSDAILLSDILTLSDENTRAPLSASMIKRYEEHQEDLALLKKLVKEQ-MPEKYWEIFSNAKK | 358 |
| EFR44625 | 232 | LLAEIGDHYADLFLAAKNLSDAILLSDILILSDENTRAPLSASMIKRYEEHQEDLALLKKLVKEQ-MPEKYWEIFSNAKK | 310 |
| WP_002897477 | 278 | LLGQIGDDFADLFLIAKKLYDAILLSGILTVTDPSTKAPLSASMIERYENHQKDLAALKQFIKNN-LPEKYVEVFSDQSK | 356 |
| WP_002906454 | 278 | LLGQIGDGFADLFLVAKKLYDAILLSGILTVTDPSTKAPLSASMIERYENHQEDLAALKQFIKNN-LSEKYAEVFSDQSK | 356 |
| WP_009729476 | 279 | LLGQIGDDFADLFLVAKKLYDAILLSGILTVINPSTKAPLSASMIERYENHQKDLASLKQFIKNN-LPEKYDEVFSDQSE | 357 |
| COR24647 | 278 | LLGIIGDEYADVFVAAKKVYDSILLSGILTTNNHSTKAPLSASMIDRYDEHNSDKKLLRDFIRTNIGKEVFKEVFYDTSK | 357 |
| WP_000066813 | 279 | LLGQIGDDFADLFLVAKKLYDAILLSGILTVKDLSTKAPLSASMIERYENHOKDLAALKOFIQNN-LQEKYDEVESDQSK | 357 |
| WP_009754323 | 279 | LLGQIGDDFADLFLVAKKLYDAILLSGILTVTDPSTKAPLSASMIERYENHQEDLAALKQFIKNN-LPEKYAEVFSDQSK | 357 |
| WP_044674937 | 278 | LLGKIGDDYADLFIATKSLYDGILLAGILSTTDSTTKAPLSSSMVNRYEEHKKDLALLKNFIHQN-LSDSYKEVENDKLK | 356 |
| WP_044676715 | 278 | LLGKIGDDYADLFIATKSLYDGILLAGILSTTDSTTKAPLSSSMVNRYEEHQKDLALLKNFIHQN-LSDSYKEVENDKLK | 356 |
| WP_044680361 | 278 | LLGKIGDDYADLFIATKSLYDGILLAGILSTTDSTTKAPLSSSMVNRYEEHQKDLALLKNFIHON-LSDSYKEVENDKLK | 356 |
| WP_044681799 | 278 | LLGKIGDDYADLFIATKSLYDGILLAGILSTTDSTTKAPLSSSMVNRYEEHKKDLALLKNFIHQN-LSDSYKEVENDKLK | 356 |
| WP_049533112 | 278 | LLGEIGDEYADLFASAKNLYDAILLSGILTVDDNSTKAPLSASMVKRYEEHQKDLKKLKDFIKVN-APDQYNAIFKDKNK | 356 |
| WP_029090905 | 244 | LADVLADEEYDLLLTAQKIYSAIILDESMDGYEYFA-----EAKKESYRKHQEELVLVKKMLKSNaITNDERAKF---EY | 315 |
| WP_006506696 | 284 | VEKDLGE-YVEFVDALHNVYSWVELQTIMGATHTD-NASISEAMVSRYNKIHDDLKLLKDCIKNN-VPNKYFDMERNDSE | 360 |
| AIT42264 | 278 | LLAQIGDQYADLFLAAKNLSDAILLSDILRVNTEITKAPLSASMIKRYDEHHQDLTLLKALVRQQ-LPEKYKEIFFDQSK | 356 |
| WP_034440723 | 284 | LLSNIDEGYRDVFLQAKNVYNAIELSKILKTDGKETKAPLSAQMVELYNQHREDLKKYKDYIKAY-LPEKYGETFKDATK | 362 |
| AKQ21048 | 278 | LLAQIGDQYADLFLAAKNLSDAILLSDILRVNTEITKAPLSASMIKRYDEHHQDLTLLKALVRQQ-LPEKYKEIFFDQSK | 356 |
| WP_004636532 | 277 | LLGMTDDVFLDVEMAAKNVYDAVEMSAIISTDTGNSKAVLSNQMINFYDEHKVDLAQLKOFFKTH-LPDKYYECFSDPSK | 355 |
| WP_002364836 | 284 | ILAKVGDEYSDVFLAAKNVYDAVELSTILADSDKKSHAKLSSSMIVRFTEHQEDLKKFKRFIREN-CPDEYDNLFKNEQK | 362 |
| WP_016631044 | 235 | ILAKVGDEYSDVFLAAKNVYDAVELSTILADSDKKSHAKLSSSMIVRFTEHQEDLKKEKRFIREN-CPDEYDNLFKNEQK | 313 |
| EMS75795 | 21 | LLEKSGEEFRDVFLQAKKVYDAILLSDILSTKKONSKAKLSLGMIERYDSHKKDLEELKQFVKAN-LPEKTAIFFKDSSK | 99 |
| WP_002373311 | 284 | ILAKVGDEYSDVFLAAKNVYDAVELSTILADSDKKSHAKLSSSMIVRFTEHQEDLKKFKRFIREN-CPDEYDNLFKNEQK | 362 |
| WP_002378009 | 284 | ILAKVGDEYSDVFLAAKNVYDAVELSTILADSDKKSHAKLSSSMIVRFTEHQEDLKNFKRFIREN-CPDEYDNLFKNEQK | 362 |
| WP_002407324 | 284 | ILAKVGDEYSDVFLAAKNVYDAVELSTILADSDKKSHAKLSSSMIVRFTEHQEDLKNFKRFIREN-CPDEYDNLFKNEQK | 362 |
| WP_002413717 | 284 | ILAKVGDEYSDVFLAAKNVYDAVELSTILADSDKKSHAKLSSSMIVRFTEHQEDLKKFKRFIREN-CPDEYDNLFKNEQK | 362 |
| WP_010775580 | 286 | ILAKVGDEYSDVFLAAKNVYDAVELSTILADSDKKSHAKLSSSMIVRFTEHQEDLKNFKRFIREN-CPDEYDNLFKNEQK | 364 |
| WP_010818269 | 284 | ILAKVGDEYSDVFLAAKNVYDAVELSTILADSDKKSHAKLSSSMIVRFTEHQEDLKKFKRFIREN-CPDEYDNLFKNEQK | 362 |
| WP_010824395 | 284 | ILAKVGDEYSDVFLAAKNVYDAVELSTILADSDKKSHAKLSSSMIVRFTEHQEDLKNFKRFIREN-CPDEYDNLFKNEQK | 362 |
| WP_016622645 | 284 | ILAKVGDEYSDVFLAAKNVYDAVELSTILADSDKKSYAKLSSSMIVRFTEHQEDLKKEKRFIREN-CPDEYDNLFKNEQK | 362 |
| WP_033624816 | 284 | ILAKVGDEYSDVFLAAKNVYDAVELSTILADSDKKSHAKLSSSMIVRFTEHQEDLKNFKRFIREN-CPDEYDNLFKNEQK | 362 |
| WP_033625576 | 284 | ILAKVGDEYSDVFLAAKNVYDAVELSTILADSDKKSHAKLSSSMIVRFTEHQEDLKKFKRFIREN-CPDEYDNLFKNEQK | 362 |
| WP_033789179 | 284 | ILAKVGDEYSDVFLAAKNVYDAVELSTILADSDKKSHAKLSSSMIVRFTEHQEDLKNFKRFIREN-CPDEYDNLFKNEQK | 362 |
| WP_002310644 | 282 | LLEKIGDDYIDLFVQAKNVYDAVLLSEILSDSTKNTRAKLSAGMIRRYDAHKEDLVLLKRFVKEN-LPKKYRAFFGDNSV | 360 |
| WP_002312694 | 283 | LLEKIGDDYIDLFVQAKNVYDAVLLSEILSDSTKNTRAKLSAGMIRRYDAHKEDLVLLKRFVKEN-LPKKYRAFFGDNSV | 361 |
| WP_002314015 | 283 | LLEKIGDDYIDLFVQAKNVYDAVLLSEILSDSTKNTRAKLSAGMIRRYDAHKEDLVLLKRFVKEN-LPKKYRAFFGDNSV | 361 |
| WP_002320716 | 283 | LLEKIGDDYIDLFVQAKNVYDAVLLSEILSDSTKNTRAKLSAGMIRRYDAHKEDLVLLKRFVKEN-LPKKYRAFFGDNSV | 361 |
| WP_002330729 | 282 | LLEKIGDDYIDLFVQAKNVYDAVLLSEILSDSTKNTRAKLSAGMIRRYDAHKEDLVLLKRFVKEN-LPKKYRAFFGDNSV | 360 |
| WP_002335161 | 283 | LLEKIGDDYIDLFVQAKNVYDAVLLSEILSDSTKNTRAKLSAGMIRRYDAHKEDLVLLKRFVKEN-LPKKYRAFFGDNSV | 361 |
| WP_002345439 | 283 | LLEKIGDDYIDLFVQAKNVYDAVLLSEILSDSTKNTRAKLSAGMIRRYDAHKEDLVLLKRFVKEN-LPKKYRAFFGDNSV | 361 |
| WP_034867970 | 276 | LLEKIGDEYLDIFLQAKKVHDAILLSEIISSTVKHTKAKLSSGMVERYERHKADLAKFKQFVKEN-VPQKATVFFKDTTK | 354 |
| WP_047937432 | 283 | LLEKIGDDYIDLFVQAKNVYDAVLLSEILSDSTKNTRAKLSAGMIRRYDAHKEDLVLLKRFVKEN-LPKKYRAFFGDNSV | 361 |
| WP_010720994 | 276 | LLEKIGDEYLDIFLQAKKVHDAILLSEIISSTVKHTQAKLSSGMVERYERHKADLAKFKQFVKEN-VPQKATVFFKDTTK | 354 |
| WP_010737004 | 276 | LLEKIGDEYLDIFLQAKKVHDAILLSEIISSTVKHTKAKLSSGMVERYERHKADLAKFKQFVKEN-VPQKATVFFKDTTK | 354 |
| WP_034700478 | 276 | LLEKIGDEYLDIFLQAKKVHDAILLSEIISSTVKHTQAKLSSGMVERYERHKADLAKFKQFVKEN-VPQKATVFFKDTTK | 354 |
| WP_007209003 | 281 | LLAIIGDEYGDIFVAAONLYQAILLAGILTSTEK-TRAKLSASMIQRYEEHAKDLKLLKRFVKEH-IPDKYAEIFNDATK | 358 |
| WP_023519017 | 276 | LLEKTSDDYAELFLKAKGVYDAILLSQILSKSDDETKAKLSANMKLRFEEHQRDLKOLKELVRRD-LPKKYDDFFKNRSK | 354 |
| WP_010770040 | 279 | LLAIIGDEYAEIFSATKSVYDAVALSGILSVTDGDTKAKLSASMVERYEAHQKDLVOFKQFIRKE-LPEMYAPIFRDNSV | 357 |
| WP_048604708 | 275 | LLGEIGDEYADVFEAAKNVYNAVELSGILTVTDNSTKAKLSASMIKRYEDHKTDLKLFKEFIRKN-LPEKYHEIFNDKNT | 353 |
| WP_010750235 | 276 | LLRKSNEEMIDVFLQVKKVYDAILLSDILSTKMKDTKAKLSAGMIERYQNHKKDLEELKOFVRAH-LHEKVTVFFKDSSK | 354 |
| AII16583 | 317 | LLAQIGDQYADLFLAAKNLSDAILLSDILRVNTEITKAPLSASMIKRYDEHHODLTLLKALVROQ-LPEKYKEIFFDQSK | 395 |
| WP_029073316 | 290 | KOPLLGD-CVEFIDLLHDIYSWVELQNILGSAHTS-EPSISAAMIQRYEDHKNDLKLLKDVIRKY-LPKKYFEVERDEKS | 366 |
| WP_031589969 | 290 | KQPLLGD-CVEFIDLLHDIYSWVELQNILGSAHTS-EPSISAAMIQRYEDHKNDLKLLKDVIRKY-LPKKYFEVERDEKS | 366 |
| KDA45870 | 276 | LLAEAGDQYLDIFVAAKKVYDAAILASILDVKDTQTKTVESQAMIERYEEHQKDLIELKRVFKKY-LPEKCHDFFSE-PK | 353 |
| WP_039099354 | 286 | LESSLDDNAHQIIESLQELYSGVLLAGIVPENQSLS-----QAMITKYDDHQKHLKMLKAVREAL-APEDRORLKQAYDQ | 359 |
| AKP02966 | 279 | LDSILDDDQFTVLDTANRIYSTITLNEIL-----NGESYFSMAKVNQYENHAIDLCKLRDMWHTT----KNEKAV-GLSR | 348 |
| WP_010991369 | 284 | LLALIGDEYAELFVAAKNAYSAVVLSSIITVAETETNAKLSASMIERFDTHEEDLGELKAFIKLH-LPKHYEEIFSNTEK | 362 |
| WP_033838504 | 284 | LLALIGDEYAELFVAAKNAYSAVVLSSIITVAETETNAKLSASMIERFDTHEEDLGELKAFIKLH-LPKHYEEIFSNTEK | 362 |
| EHN60060 | 287 | LLALIGDEYAELFVAAKNAYSAVVLSSIITVAETETNAKLSASMIERFDTHEEDLGELKAFIKLH-LPKHYEEIFSNTEK | 365 |
| EFR89594 | 53 | LLALIGDEYAELFVAAKNAYSAVVLSSIITVAETETNAKLSASMIERFDTHEEDLGELKAFIKLH-LPKHYEEIFSNTEK | 131 |
| WP_038409211 | 284 | LLAKIGDEYAEIFVAAKSTYNAVVLSNIITVTDTETKAKLSASMIERFDKHAKDLKRLKAFFKMQ-LPEKFNEVENDIEK | 362 |
| EFR95520 | | -------------------------------------------------------------------------------- | |
| WP_003723650 | 284 | LLAIIGDEYAELFVAAKNTYNAVVLSSIITVTDTETNAKLSASMIERFDAHEKDLVELKAFIKLN-LPKQYEEIFSNAAI | 362 |
| WP_003727705 | 284 | LLAIIGDEYAELFVAAKNTYNAVVLSSIITVTATETNAKLSASMIERFDAHEKELGELKAFIKLH-LPKQYQEIFNNAEI | 362 |
| WP_003730785 | 284 | LLAIIGDEYAELFVAAKNTYNAVVLSSIITVTATETNAKLSASMIERFDAHEKELGELKAFIKLH-LPKQYQEIFNNAEI | 362 |
| WP_003733029 | 284 | LLAIIGDEFAEVFVAAKNAYNAVVLSNIITVIDSTTRAKLSASLIERFENHKEDLKKMKRFVRTY-LPEKYDEIFDDTEK | 362 |
| WP_003739838 | 284 | LLAIIGDEYAELFVAAKNTYNAVVLSSIITVTDTETNAKLSASMIERFDAHEKDLSELKAFIKLH-LPKQYEEIFSNVAI | 362 |
| WP_014601172 | 284 | LLAIIGDEYAELFVAAKNTYNAVVLSSIITVTATETNAKLSASMIERFDAHEKDLGELKAFIKLH-LPKQYQEIFNNAAI | 362 |
| WP_023548323 | 284 | LLAIIGDEYAELFVAAKNTYNAVVLSSIITVTDTETNAKLSASMIERFDAHEKDLVELKAFIKLN-LPKQYEEIFSNAAI | 362 |
| WP_031665337 | 284 | LLAIIGDEYAELFVAAKNTYNAVVLSSIITVNDTETNAKLSASMIERFDAHEKDLVELKAFIKLN-LPKQYEEIFSNAAI | 362 |
| WP_031669209 | 284 | LLAIIGDEFAEVFVAAKNAYNAVVLSNIITVTDSTTRAKLSASLIERFENHKEDLKKMKRFVRTY-LPEKYDEIFDDTEK | 362 |
| WP_033920898 | 284 | LLAIIGDEYAELFVAAKNTYNAVVLSSIITVTDTETNAKLSASMIERFDAHEKDLVELKAFIKLN-LPKQYEEIFSNAAI | 362 |
| AKI42028 | 287 | LLAIIGDEYAELFVAAKNTYNAVVLSSIITVTATETNAKLSASMIERFDAHEKDLGELKAFIKLH-LPKQYQEIFNNAAI | 365 |
| AKI50529 | 287 | LLAIIGDEYAELFVAAKNTYNAVVLSSIITVTDTETNAKLSASMIERFDAHEKDLVELKAFIKLN-LPKQYEEIFSNAAI | 365 |
| EFR83390 | | -------------------------------------------------------------------------------- | |
| WP_046323366 | 284 | LLARVGDEYAEIFVAAKNAYNAVVLSSIITVSNTETKAKLSASMIERFDKHDKDLKRMKAFFKVR-LPENFNEVENDVEK | 362 |
| AKE81011 | 294 | LLAQIGDQYADLFLAAKNLSDAILLSDILRVNTEITKAPLSASMIKRYDEHHQDLTLLKALVRQQ-LPEKYKEIFFDQSK | 372 |
| CUO82355 | 288 | VENDLGE-YVEFIDSLHNIYSWVELQTIMGATHTD-NASISEAMVSRYNKIHEDLOLLKKCIKDN-VPKKYFDMFRNDSE | 364 |
| WP_033162887 | 289 | LQSELGE-YIEFIEMLHNIYSWVELQAILGATHTD-NPSISAAMVERYEEHKKDLRVLKKVIREE-LPDKYNEVERKDNR | 365 |
| AGZ01981 | 311 | LLAQIGDQYADLFLAAKNLSDAILLSDILRVNTEITKAPLSASMIKRYDEHHQDLTLLKALVRQQ-LPEKYKEIFFDQSK | 389 |
| AKA60242 | 278 | LLAQIGDQYADLFLAAKNLSDAILLSDILRVNTEITKAPLSASMIKRYDEHHQDLILLKALVRQQ-LPEKYKEIFFDQSK | 356 |
| AKS40380 | 278 | LLAQIGDQYADLFLAAKNLSDAILLSDILRVNTEITKAPLSASMIKRYDEHHQDLTLLKALVRQQ-LPEKYKEIFFDQSK | 356 |
| 4UN5_B | 282 | LLAQIGDQYADLFLAAKNLSDAILLSDILRVNTEITKAPLSASMIKRYDEHHQDLTLLKALVROQ-LPEKYKEIFFDQSK | 360 |
| WP_010922251 | 357 | 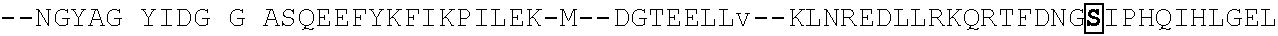 | 419 |
| WP_039695303 | 358 | --NGYAG YIEN G VKQDEFYKYLKNILSK-IkiDGSDYFL--DKIEREDFLRKORTFDNGSIPHQIHLQEM | 422 |
| WP_045635197 | 357 | --DGYAG YIDG K TTQETFYKYIKNLLSK-F--EGTDYFL--DKIEREDFLRKORTFDNGSIPHQIHLQEM | 419 |
| 5AXW_A | 169 | ------G SINR - ---------------K------TSDYVk----------------------------EA | 183 |
| WP_009880683 | 41 | --NGYAG YIDG G ASQEEFYKFIKPILEK-M--DGTEELLa--KLNREDLLRKORTFDNGSIPHQIHLGEL | 103 |
| WP_010922251 | 357 | --NGYAG YIDG G ASQEEFYKFIKPILEK-M--DGTEELLv--KLNREDLLRKORTFDNGSIPHQIHLGEL | 419 |
| WP_011054416 | 357 | --NGYAG YIDG G ASQEEFYKFIKPILEK-M--DGTEELLa--KLNREDLLRKORTFDNGSIPYQIHLGEL | 419 |
| WP_011284745 | 357 | --NGYAG YIDG G ASQEEFYKFIKPILEK-M--DGTEELLa--KLNREDLLRKORTFDNGSIPHQIHLGEL | 419 |
| WP_011285506 | 357 | --NGYAG YIDG G ASQEEFYKFIKPILEK-M--DGTEELLv--KLNREDLLRKORTFDNGSIPHQIHLGEL | 419 |
| WP_011527619 | 357 | --NGYAG YIDG G ASQEEFYKFIKPILEK-M--DGTEELLa--KLNREDLLRKORTFDNGSIPHQIHLGEL | 419 |
| WP_012560673 | 357 | --NGYAG YIDG G ASQEEFYKFIKPILEK-M--DGTEELLa--KLNREDLLRKORTFDNGSIPHQIHLGEL | 419 |
| WP_014407541 | 357 | --NGYAG YIDG G ASQEEFYKFIKPILEK-M--DGTEELLv--KLNREDLLRKORTEDNGSIPHQIHLGEL | 419 |
| WP_020905136 | 357 | --NGYAG YIDG G ASQEEFYKFIKPILEK-M--DGTEELLa--KLNREDLLRKORTFDNGSIPHQIHLGEL | 419 |
| WP_023080005 | 357 | --NGYAG YIDG G ASQEEFYKFIKPILEK-M--DGTEELLa--KLNREDLLRKORTFDNGSIPYQIHLGEL | 419 |
| WP_023610282 | 357 | --NGYAG YIDG G ASQEEFYKFIKPILEK-M--DGTEELLa--KLNREDLLRKORTFDNGSIPYQIHLGEL | 419 |
| WP_030125963 | 357 | --NGYAG YIDG G ASQEEFYKFIKPILEK-M--DGTEELLv--KLNREDLLRKORTEDNGSIPHOIHLGEL | 419 |
| WP_030126706 | 357 | --NGYAG YIDG G ASQEEFYKFIKPILEK-M--DGTEELLa--KLNREDLLRKORTEDNGSIPYQIHLGEL | 419 |
| WP_031488318 | 357 | --NGYAG YIDG G ASQEEFYKFIKPILEK-M--DGTEELLa--KLNREDLLRKORTFDNGSIPHQIHLGEL | 419 |
| WP_032460140 | 357 | --NGYAG YIDG G ASQEEFYKFIKPILEK-M--DGTEELLa--KLNREDLLRKORTFDNGSIPHQIHLGEL | 419 |
| WP_032461047 | 357 | --NGYAG YIDG G ASQEEFYKFIKPILEK-M--DGTEELLa--KLNREDLLRKORTFDNGSIPHQIHLGEL | 419 |
| WP_032462016 | 357 | --NGYAG YIDG G ASQEEFYKFIKPILEK-M--DGTEELLv--KLNREDLLRKORTFDNGSIPHQIHLGEL | 419 |
| WP_032462936 | 357 | --NGYAG YIDG G ASQEEFYKFIKPILEK-M--DGTEELLa--KLNREDLLRKORTFDNGSIPHQIHLGEL | 419 |
| WP_032464890 | 357 | --NGYAG YIDG G ASQEEFYKFIKPILEK-M--DGTEELLa--KLNRKDLLRKORTFDNGSIPHQIHLGEL | 419 |
| WP_033888930 | 182 | --NGYAG YIDG G ASQEEFYKFIKPILEK-M--DGTEELLv--KLNREDLLRKORTFDNGSIPHQIHLGEL | 244 |
| WP_038431314 | 357 | --NGYAG YIDG G ASQEEFYKFIKPILEK-M--DGTEELLa--KLNREDLLRKORTFDNGSIPHQIHLGEL | 419 |
| WP_038432938 | 357 | --NGYAG YIDG G ASQEEFYKFIKPILEK-M--DGTEELLa--KLNREDLLRKORTFDNGSIPYQIHLGEL | 419 |
| WP_038434062 | 357 | --NGYAG YIDG G ASQEEFYKFIKPILEK-M--DGTEELLa--KLNREDLLRKQRTFDNGSIPHQIHLGEL | 419 |
| BAQ51233 | 268 | --NGYAG YIDG G ASQEEFYKFIKPILEK-M--DGTEELLv--KLNREDLLRKORTFDNGSIPHQIHLGEL | 330 |
| KGE60162 | | ------- ---- - ---------------------------------------------------------- | |
| KGE60856 | | ------- ---- - ---------------------------------------------------------- | |
| WP_002989955 | 357 | --NGYAG YIDG G ASQEEFYKFIKPILEK-M--DGTEELLa--KLNREDLLRKORTFDNGSIPHQIHLGEL | 419 |
| WP_003030002 | 357 | --KGYAG YIEN G VKQDEFYKYLKGILLQ-I--NGSGDFL--DKIDREDFLRKORTFDNGSIPHQIHLQEM | 419 |
| WP_003065552 | 360 | --NGYAG YIEN G VKQDEFYKYLKNTLSK-Ia--GSDYFL--DKIEREDFLRKORTFDNGSIPHQIHLQEM | 422 |
| WP_001040076 | 358 | --DGYAG YIEG K TNQEAFYKYLSKLLTK-Q--EGSEYFL--EKIKNEDFLRKORTFDNGSIPHQVHLTEL | 420 |
| WP_001040078 | 358 | --DGYAG YIEG K TNQEAFYKYLSKLLTK-Q--EDSENFL--EKIKNEDFLRKORTFDNGSIPHQVHLTEL | 420 |
| WP_001040080 | 358 | --DGYAG YIEG K TNQEAFYKYLSKLLTK-Q--EDSENFL--EKIKNEDFLRKORTFDNGSIPHQVHLTEL | 420 |
| WP_001040081 | 358 | --DGYAG YIEG K TNQEAFYKYLSKLLTK-Q--EDSENFL--EKIKNEDFLRKORTFDNGSIPHQVHLTEL | 420 |
| WP_001040083 | 358 | --DGYAG YIEG K TNQEAFYKYLSKLLTK-Q--EDSENFL--EKIKNEDFLRKORTFDNGSIPHQVHLTEL | 420 |
| WP_001040085 | 358 | --DGYAG YIEG K TNQEAFYKYLSKLLTK-Q--EDSENFL--EKIKNEDFLRKORTFDNGSIPHQVHLTEL | 420 |
| WP_001040087 | 358 | --DGYAG YIEG K TNQEAFYKYLSKLLTK-Q--EDSENFL--EKIKNEDFLRKORTFDNGSIPHQVHLTEL | 420 |
| WP_001040088 | 358 | --DGYAG YIEG K TNQEAFYKYLSKLLTK-Q--EDSENFL--EKIKNEDFLRKORTFDNGSIPHQVHLTEL | 420 |
| WP_001040089 | 358 | --DGYAG YIEG K TNQEAFYKYLSKLLTK-Q--EDSENFL--EKIKNEDFLRKORTFDNGSIPHQVHLTEL | 420 |
| WP_001040090 | 358 | --DGYAG YIEG K TNQEAFYKYLSKLLTK-Q--EDSENFL--EKIKNEDFLRKORTFDNGSIPHQVHLTEL | 420 |
| WP_001040091 | 358 | --DGYAG YIEG K TNQEAFYKYLSKLLTK-Q--EDSENFL--EKIKNEDFLRKORTFDNGSIPHQVHLTEL | 420 |
| WP_001040092 | 358 | --DGYAG YIEG K TNQEAFYKYLSKLLTK-Q--EDSEYFL--EKIKNEDFLRKORTFDNGSIPHQVHLTEL | 420 |
| WP_001040094 | 358 | --DGYAG YIEG K TNQEAFYKYLSKLLTK-Q--EGSEYLL--EKIKNEDFLRKORTFDNGSIPHQVHLTEL | 420 |
| WP_001040095 | 358 | --DGYAG YIEG K TNQEAFYKYLSKLLTK-Q--EGSEYLL--EKIKNEDFLRKORTFDNGSIPHQVHLTEL | 420 |
| WP_001040096 | 358 | --DGYAG YIEG K TNQEAFYKYLSKLLTK-Q--EGSEYLL--EKIKNEDFLRKORTFDNGSIPHQVHLTEL | 420 |
| WP_001040097 | 358 | --DGYAG YIEG K TNQEAFYKYLSKLLTK-Q--EGSEYLL--EKIKNEDFLRKORTFDNGSIPHQVHLTEL | 420 |
| WP_001040098 | 358 | --DGYAG YIEG K TNQEAFYKYLSKLLTK-Q--EGSEYLL--EKIKNEDFLRKORTFDNGSIPHQVHLTEL | 420 |
| WP_001040099 | 358 | --DGYAG YIEG K TNQEAFYKYLSKLLTK-Q--EGSEYLL--EKIKNEDFLRKORTFDNGSIPHQVHLTEL | 420 |
| WP_001040100 | 358 | --DGYAG YIEG K TNQEAFYKYLSKLLTK-Q--EGSEYLL--EKIKNEDFLRKORTFDNGSIPHQVHLTEL | 420 |
| WP_001040104 | 358 | --DGYAG YIEG K TNQEAFYKYLSKLLTK-Q--EDSENFL--EKIKNEDFLRKORTFDNGSIPHQVHLTEL | 420 |
| WP_001040105 | 358 | --DGYAG YIEG K TNQEAFYKYLSKLLTK-Q--EDSENFL--EKIKNEDFLRKORTFDNGSIPHQVHLTEL | 420 |
| WP_001040106 | 358 | --DGYAG YIEG K TNQGAFYKYLSKLLTK-Q--EGSEYFL--EKIKNEDFLRKORTFDNGSIPHQVHLTEL | 420 |
| WP_001040107 | 358 | --DGYAG YIEG K TNQGAFYKYLSKLLTK-Q--EGSEYFL--EKIKNEDFLRKORTFDNGSIPHQVHLTEL | 420 |
| WP_001040108 | 358 | --DGYAG YIEG K TNQGAFYKYLSKLLTK-Q--EGSEYFL--EKIKNEDFLRKORTEDNGSIPHQVHLTEL | 420 |
| WP_001040109 | 358 | --DGYAG YIEG K TNQGAFYKYLSKLLTK-Q--EGSEYFL--EKIKNEDFLRKORTFDNGSIPHQVHLTEL | 420 |
| WP_001040110 | 358 | --DGYAG YIEG K TNQGAFYKYLSKLLTK-Q--EGSEYFL--EKIKNEDFLRKORTFDNGSIPHQVHLTEL | 420 |
| WP_015058523 | 358 | --DGYAG YIEG K TNQEAFYKYLSKLLTK-Q--EDSEYFL--EKIKNEDFLRKORTFDNGSIPHQVHLTEL | 420 |
| WP_017643650 | 358 | --DGYAG YIEG K TNQEAFYKYLSKLLTK-Q--EGSEYLL--EKIKNEDFLRKORTFDNGSIPHQVHLTEL | 420 |
| WP_017647151 | 358 | --DGYAG YIEG K TNQGAFYKYLSKLLTK-Q--EGSEYFL--EKIKNEDFLRKORTFDNGSIPHQVHLTEL | 420 |
| WP_017648376 | 358 | --DGYAG YIEG K TNQGAFYKYLSKLLTK-Q--EGSEYFL--EKIKNEDFLRKORTFDNGSIPHQVHLTEL | 420 |
| WP_017649527 | 358 | --DGYAG YIEG K TNQEAFYKYLSKLLTK-Q--EDSENFL--EKIKNEDFLRKORTFDNGSIPHQVHLTEL | 420 |
| WP_017771611 | 358 | --DGYAG YIEG K TNQGAFYKYLSKLLTK-Q--EGSEYFL--EKIKNEDFLRKORTFDNGSIPHQVHLTEL | 420 |
| WP_017771984 | 358 | --DGYAG YIEG K TNQEAFYKYLSKLLTK-Q--EDSENFL--EKIKNEDFLRKORTFDNGSIPHQVHLTEL | 420 |
| CFQ25032 | 358 | --DGYAG YIEG K TNQEAFYKYLSKLLTK-Q--EDSENFL--EKIKNEDFLRKORTFDNGSIPHQVHLTEL | 420 |
| CFV16040 | 358 | --DGYAG YIEG K TNQEAFYKYLSKLLTK-Q--EDSENFL--EKIKNEDFLRKORTFDNGSIPHQVHLTEL | 420 |
| KLJ37842 | 358 | --DGYAG YIEG K TNQEAFYKYLSKLLTK-Q--EDSENFL--EKIKNEDFLRKORTFDNGSIPHQVHLTEL | 420 |
| KLJ72361 | 358 | --DGYAG YIEG K TNQEAFYKYLSKLLTK-Q--EDSENFL--EKIKNEDFLRKORTFDNGSIPHQVHLTEL | 420 |
| KLL20707 | 358 | --DGYAG YIEG K TNQEAFYKYLSKLLTK-Q--EDSENFL--EKIKNEDFLRKORTFDNGSIPHQVHLTEL | 420 |
| KLL42645 | 358 | --DGYAG YIEG K TNQGAFYKYLSKLLTK-Q--EGSEYFL--EKIKNEDFLRKORTFDNGSIPHQVHLTEL | 420 |
| WP_047207273 | 358 | --DGYAG YIEG K TNQEAFYKYLSKLLTK-Q--EDSENFL--EKIKNEDFLRKORTEDNGSIPHQVHLTEL | 420 |
| WP_047209694 | 358 | --DGYAG YIEG K TNQEAFYKYLSKLLTK-Q--EGSEYLL--EKIKNEDFLRKORTFDNGSIPHQVHLTEL | 420 |
| WP_050198062 | 358 | --DGYAG YIEG K TNQEAFYKYLSKLLTK-Q--EDSENFL--EKIKNEDFLRKORTFDNGSIPHQVHLTEL | 420 |
| WP_050201642 | 358 | --DGYAG YIEG K TNQEAFYKYLSKLLTK-Q--EDSENFL--EKIKNEDFLRKORTFDNGSIPHQVHLTEL | 420 |
| WP_050204027 | 358 | --DGYAG YIES K TNQGAFYKYLSKLLTK-Q--EGSEYFL--EKIKNEDFLRKORTFDNGSIPHQVHLTEL | 420 |
| WP_050881965 | 358 | --DGYAG YIEG K TNQEAFYKYLSKLLTK-Q--EDSENFL--EKIKNEDFLRKORTFDNGSIPHQVHLTEL | 420 |
| WP_050886065 | 358 | --DGYAG YIEG K TNQEAFYKYLSKLLTK-Q--EDSENFL--EKIKNEDFLRKORTFDNGSIPHQVHLTEL | 420 |
| AHN30376 | 358 | --DGYAG YIEG K TNQEAFYKYLSKLLTK-Q--EDSEYFL--EKIKNEDFLRKORTFDNGSIPHQVHLTEL | 420 |
| EA078426 | 358 | --DGYAG YIEG K TNQEAFYKYLSKLLTK-Q--EDSENFL--EKIKNEDFLRKORTFDNGSIPHQVHLTEL | 420 |
| CCW42055 | 358 | --DGYAG YIEG K TNQEAFYKYLSKLLTK-Q--EGSEYLL--EKIKNEDFLRKORTEDNGSIPHQVHLTEL | 420 |
| WP_003041502 | 357 | --KGYAG YIES G VKQDEFYKYLKGILLQ-I--NGSGDFL--DKIDREDFLRKORTFDNGSIPHQIHLQEM | 419 |
| WP_037593752 | 358 | --KGYAG YIES G VEQDEFYKYLKGILLK-I--NGSGDFL--DKIDCEDFLRKORTFDNGSIPHQIHLQEM | 420 |
| WP_049516684 | 358 | --NGYAG YIEN G VKQDEFYKYLKNTLSK-I--DGSDYFL--DKIDREDFLRKORTFDNGSIPHQIHLQEM | 420 |
| GAD46167 | 357 | --KGYAG YIES G VEQDEFYKYLKGILLK-I--NGSGDFL--DKIDCEDFLRKORTFDNGSIPHQIHLQEM | 419 |
| WP_018363470 | 358 | --NGYAG YIEN G VKQDEFYKYLKGILTK-I--NGSDYFL--DKIEREDFLRKORTFDNGSIPHQIHLQEM | 420 |
| WP_003043819 | 357 | --NGYAG YVGI G ATQEEFYKFIKPILEK-M--DGAEELLa--KLNRDDLLRKORTFDNGSIPHQIHLKEL | 429 |
| WP_006269658 | 357 | --KGYAS YIES G VKQDEFYKYLKGILLK-I--NGSGDFL--DKIDREDFLRKORTFDNGSIPHQIHLQEM | 419 |
| WP_048800889 | 357 | --NGYAG YIEN G VKQDEFYKYLKNTLSK-I--DGSGYFL--DKIEREDFLRKORTFDNGSIPHQIHLQEM | 419 |
| WP_012767106 | 357 | --NGYAG YIDG G ASQEEFYKFIKPILEK-M--DGTEELLa--KLNREDLLRKORTFDNGSIPHQIHLGEL | 419 |
| WP_014612333 | 357 | --NGYAG YIDG G ASQEEFYKFIKPILEK-M--DGTEELLa--KLNREDLLRKORTFDNGSIPHQIHLGEL | 419 |
| WP_015017095 | 357 | --NGYAG YIDG G ASQEEFYKFIKPILEK-M--DGTEELLa--KLNREDLLRKORTFDNGSIPHQIHLGEL | 419 |
| WP_015057649 | 357 | --NGYAG YIDG G ASQEEFYKFIKPILEK-M--DGTEELLa--KLNREDLLRKORTFDNGSIPHQIHLGEL | 419 |
| WP_048327215 | 357 | --NGYAG YIDG G ASQEEFYKFIKPILEK-M--DGTEELLa--KLNREDLLRKORTFDNGSIPHQIHLGEL | 419 |
| WP_049519324 | 357 | --NGYAG YIDG G ASQEEFYKFIKPILEK-M--DGTEELLa--KLNREDLLRKORTFDNGSIPHQIHLGEL | 419 |
| WP_012515931 | 357 | --NGYAG YIEG Q VSQEEFYKYLKPILAR-L--DGSEPLL1--KIDREDFLRKORTFDNGSIPHQIHLEEL | 419 |
| WP_021320964 | 357 | --NGYAG YIEG 2 VSQEEFYKYLKPILAR-L--DGSEPLL1--KIDREDFLRKORTEDNGSIPHQIHLEEL | 419 |
| WP_037581760 | 357 | --NGYAG YIEG Q VSQEEFYKYLKPILAR-L--DGSEPLL1--KIDREDFLRKORTEDNGSIPHQIHLEEL | 419 |
| WP_004232481 | 357 | --NGYAG YIEN G VKQDIFYKHLKSIISE-K--NGGQYFL--DKIEREDFLRKORTFDNGSIPYQIHLQEM | 419 |
| WP_009854540 | 358 | --NGYAG YIEN G VKQDEFYKYLKNTLSK-I--DGSDYFL--DKIEREDFLRKORTFDNGSIPHQIHLQEM | 420 |
| WP_012962174 | 358 | --NGYAG YIEN G VKQDEFYKYLKTTLSK-I--DGSDYFL--DKIEREDFLRKORTFDNGSIPHQIHLQEM | 420 |
| WP_039695303 | 358 | --NGYAG YIEN G VKQDEFYKYLKNILSK-IkiDGSDYFL--DKIEREDFLRKORTFDNGSIPHQIHLQEM | 422 |
| WP_014334983 | 357 | --NGYAG YIDN G VKQDEFYKYLKTILTK-I--DDSDYFL--DKIERDDFLRKORTFDNGSIPHQIHLQEM | 419 |
| WP_003099269 | 357 | --NGYAG YIDG K TSQEEFYKYIKPILLK-L--DGTEKLIS--KLEREDFLRKORTEDNGSIPHQIHLNEL | 419 |
| AHY15608 | 357 | --NGYAG YIDG K TSQEEFYKYIKPILLK-L--DGTEKLIS--KLEREDFLRKORTFDNGSIPHQIHLNEL | 419 |
| AHY17476 | 357 | --NGYAG YIDG K TSQEEFYKYIKPILLK-L--DGTEKLIS--KLEREDFLRKORTFDNGSIPHQIHLNEL | 419 |
| ESR09100 | | ------- ---- - --------------------------------------------------------- | |
| AGM98575 | 357 | --NGYAG YIDG K TSQEEFYKYIKPILLK-L--DGTEKLIs--KLEREDFLRKORTFDNGSIPHQIHLNEL | 419 |
| ALF27331 | 357 | --DGYAG YIDG K TNQEAFYKYLKGLLNK-I--EGSGYFL--DKIEREDFLRKORTFDNGSIPHQIHLQEM | 419 |
| WP_018372492 | 355 | --PSYAA YVSG A VTEDDFYKFSKGLLID-V--EGAEYFL--EKIEREDFLRKORTFDNGAIPNQVHVKEL | 432 |
| WP_045618028 | 358 | --DGYAG YIDG K TTQEAFYKYIKNLLSK-L--EGADYFL--DKIEREDFLRKORTFDNGSIPHQIHLQEM | 420 |
| WP_045635197 | 357 | --DGYAG YIDG K TTQETFYKYIKNLLSK-F--EGTDYFL--DKIEREDFLRKORTFDNGSIPHQIHLQEM | 419 |
| WP_002263549 | 357 | --DGYAG YIDG K TNQEAFYKYLKGLLNK-I--EGSGYFL--DKIEREDFLRKORTFDNGSIPHQIHLQEM | 419 |
| WP_002263887 | 357 | --DGYAG YIDG K TNQEAFYKYLKGLLNK-I--EGSGYFL--DKIEREDFLRKORTFDNGSIPHQIHLQEM | 419 |
| WP_002264920 | 357 | --NGYAG YIEN G VKQDEFYKYLKNTLSK-I--AGSDYFL--DKIEREDFLRKORTFDNGSIPHQIHLQEM | 419 |
| WP_002269043 | 357 | --DGYAG YIDG K TNQEAFYKYLKGLLNK-I--EGSGYFL--DKIEREDFLRKORTFDNGSIPHQIHLQEM | 419 |
| WP_002269448 | 357 | --DGYAG YIDG K TNQEAFYKYLKGLLNK-I--EGSGYFL--DKIEREDFLRKORTFDNGSIPHQIHLQEM | 419 |
| WP_002271977 | 357 | --DGYAG YIDG K TNQEAFYKYLKGLLNK-I--EGSGYFL--DKIEREDFLRKQRTFDNGSIPHQIHLQEM | 419 |
| WP_002272766 | 357 | --DGYAG YIDG K TNQEAFYKYLKGLLNK-I--EGSGYFL--DKIEREDFLRKORTFDNGSIPHQIHLQEM | 419 |
| WP_002273241 | 357 | --DGYAG YIDG K TNQEAFYKYLKGLLNK-I--EGSGYFL--DKIEREDFLRKORTFDNGSIPHQIHLQEM | 419 |
| WP_002275430 | 357 | --DGYAG YIDG K TNQEAFYKYLKGLLNK-I--EGSGYFL--DKIEREDFLRKORTFDNGSIPHQIHLQEM | 419 |
| WP_002276448 | 357 | --DGYAG YIDG K TNQEAFYKYLKGLLNK-I--EGSGYFL--DKIEREDFLRKORTFDNGSIPHQIHLQEM | 419 |
| WP_002277050 | 357 | --NGYAG YIEN G VKQDEFYKYLKNTLSK-I--AGSDYFL--DKIEREDFLRKORTFDNGSIPHQIHLQEM | 419 |
| WP_002277364 | 357 | --DGYAG YIDG K TNQEAFYKYLKGLLNK-I--EGSGYFL--DKIEREDFLRKORTFDNGSIPHQIHLQEM | 419 |
| WP_002279025 | 357 | --DGYAG YIDG K TNQEAFYKYLKGLLNK-I--EGSGYFL--DKIEREDFLRKORTFDNGSIPHQIHLQEM | 419 |
| WP_002279859 | 357 | --DGYAG YIDG K TNQEAFYKYLKGLLNK-I--EGSGYFL--DKIEREDFLRKORTFDNGSIPHQIHLQEM | 419 |
| WP_002280230 | 357 | --DGYAG YIDG K TNQEAFYKYLKGLLNK-I--EGSGYFL--DKIEREDFLRKORTFDNGSIPHQIHLQEM | 419 |
| WP_002281696 | 357 | --DGYAG YIDG K TNQEAFYKYLKGLLNK-I--EGSGYFL--DKIEREDFLRKORTFDNGSIPHQIHLQEM | 419 |
| WP_002282247 | 357 | --NGYAG YIEN G VKQDEFYKYLKNTLSK-I--TGSDYFL--DQIEREDFLRKORTFDNGSIPHQIHLQEM | 419 |
| WP_002282906 | 357 | --DGYAG YIDG K TNQEAFYKYLKGLLNK-I--EGSGYFL--DKIEREDFLRKORTFDNGSIPHQIHLQEM | 419 |
| WP_002283846 | 357 | --DGYAG YIDG K TNQEAFYKYLKGLLNK-I--EGSGYFL--DKIEREDFLRKORTFDNGSIPHQIHLQEM | 419 |
| WP_002287255 | 357 | --DGYAG YIDG K TNQEAFYKYLKGLLNK-I--EGSGYFL--DKIEREDFLRKORTFDNGSIPHQIHLQEM | 419 |
| WP_002288990 | 357 | --DGYAG YIDG K TNQEAFYKYLKGLLNK-I--EGSGYFL--DKIEREDFLRKORTFDNGSIPHQIHLQEM | 419 |
| WP_002289641 | 357 | --DGYAG YIDG K TNQEAFYKYLKGLLNK-I--EGSGYFL--DKIEREDFLRKORTFDNGSIPHQIHLQEM | 419 |
| WP_002290427 | 357 | --DGYAG YIDG K TNQEAFYKYLKGLLNK-I--EGSGYFL--DKIEREDFLRKORTFDNGSIPHQIHLQEM | 419 |
| WP_002295753 | 357 | --DGYAG YIDG K TNQEAFYKYLKGLLNK-I--EGSGYFL--DKIEREDFLRKORTFDNGSIPHQIHLQEM | 419 |
| WP_002296423 | 357 | --DGYAG YIDG K TNQEAFYKYLKGLLNK-I--EGSGYFL--DKIEREDFLRKORTFDNGSIPHQIHLQEM | 419 |
| WP_002304487 | 357 | --NGYAG YVGA D ATEEEFYKYVKGILNK-V--EGADVWL--DKIDREDFLRKORTFDNGSIPHQIHLQEM | 429 |
| WP_002305844 | 357 | --DGYAG YIDG K TNQEAFYKYLKGLLNK-I--EGSGYFL--DKIEREDFLRKORTFDNGSIPHQIHLQEM | 419 |
| WP_002307203 | 357 | --DGYAG YIDG K TNQEAFYKYLKGLLNK-I--EGSGYFL--DKIEREDFLRKORTFDNGSIPHQIHLQEM | 419 |
| WP_002310390 | 357 | --DGYAG YIDG K TNQEAFYKYLKGLLNK-I--EGSGYFL--DKIEREDFLRKORTFDNGSIPHQIHLQEM | 419 |
| WP_002352408 | 357 | --DGYAG YIDG K TNQEAFYKYLKGLLNK-I--EGSGYFL--DKIEREDFLRKORTFDNGSIPHQIHLQEM | 419 |
| WP_012997688 | 357 | --DGYAG YIDG K TNQEAFYKYLKGLLNK-I--EGSGYFL--DKIEREDFLRKORTEDNGSIPHOIHLQEM | 419 |
| WP_014677909 | 357 | --DGYAG YIDG K TNQEAFYKYLKGLLNK-I--EGSGYFL--DKIEREDFLRKORTFDNGSIPHQIHLQEM | 419 |
| WP_019312892 | 357 | --DGYAG YIDG K TNQEAFYKYLKGLLNK-I--EGSGYFL--DKIEREDFLRKORTFDNGSIPHQIHLQEM | 419 |
| WP_019313659 | 357 | --DGYAG YIDG K TNQEAFYKYLKGLLNK-I--EGSGYFL--DKIEREDFLRKORTFDNGSIPHQIHLQEM | 419 |
| WP_019314093 | 357 | --DGYAG YIDG K TNQEAFYKYLKGLLNK-I--EGSGYFL--DKIEREDFLRKORTFDNGSIPHQIHLQEM | 419 |
| WP_019315370 | 357 | --DGYAG YIDG K TNQEAFYKYLKGLLNK-I--EGNGYFL--DKIEREDFLRKORTFDNGSIPHQIHLQEM | 419 |
| WP_019803776 | 357 | --DGYAG YIDG K TNQEAFYKYLKGLLNK-I--EGSGYFL--DKIEREDFLRKORTFDNGSIPHQIHLQEM | 419 |
| WP_019805234 | 357 | --DGYAG YIDG K TNQEAFYKYLKGLLNK-I--EGSGYFL--DKIEREDFLRKORTFDNGSIPHQIHLQEM | 419 |
| WP_024783594 | 357 | --DGYAG YIDG K TNQEAFYKYLKGLLNK-I--EGSGYFL--DKIEREDFLRKORTFDNGSIPHQIHLQEM | 419 |
| WP_024784288 | 357 | --NGYAG YIEN G VKQDEFYKYLKNTLSK-I--TGSDYFL--DQIEREDFLRKORTFDNGSIPHQIHLQEM | 419 |
| WP_024784666 | 357 | --DGYAG YIDG K TNQEAFYKYLKGLLNK-I--EGSGYFL--DKIEREDFLRKORTFDNGSIPHQIHLQEM | 419 |
| WP_024784894 | 357 | --DGYAG YIDG K TNQEAFYKYLKGLLNK-I--EGSGYFL--DKIEREDFLRKORTFDNGSIPHQIHLQEM | 419 |
| WP_024786433 | 357 | --NGYAG YIEN G VKQDEFYKYLKNTLSK-I--AGSDYFL--DKIEREDFLRKORTFDNGSIPHQIHLQEM | 419 |
| WP_049473442 | 357 | --DGYAG YIDG K TNQEAFYKYLKGLLNK-I--EGSGYFL--DKIEREDFLRKORTFDNGSIPHOIHLQEM | 419 |
| WP_049474547 | 357 | --DGYAG YIDG K TNQEAFYKYLKGLLNK-I--EGSGYFL--DKIEREDFLRKORTFDNGSIPHQIHLQEM | 419 |
| EMC03581 | 350 | --DGYAG YIDG K TNQEAFYKYLKGLLNK-I--EGSGYFL--DKIEREDFLRKORTFDNGSIPHOIHLQEM | 412 |
| WP_000428612 | 358 | --DGYAG YIDG K TTQESFYKYIKNLLSK-F--EGADYFL--EKIEREDFLRKORTFDNGSIPHQIHLQEM | 420 |
| WP_000428613 | 358 | --DGYAG YIDG K TTQEAFYKYIKNLLSK-F--EGTDYFL--EKIEREDFLRKORTFDNGSIPHQIHLQEM | 420 |
| WP_049523028 | 357 | --DGYAG YIDG K TTQEGFYKYIKNLISK-I--EGAEYFL--EKIEREDFLRKORTFDNGSIPHQIHLQEM | 419 |
| WP_003107102 | 326 | --NGYAG YING K TSQEDFYKYIKPILSK-L--KGAESLIs--KLEREDFLRKQRTFDNGSIPHQIHLNEL | 388 |
| WP_054279288 | 359 | --DGYAG YISG K TSQEAFYKYIKPILET-L--DGAEDFLt--KINREDFLRKORTFDNGSIPHQIHLGEL | 421 |
| WP_049531101 | 358 | --EGYAG YIDS K TTQEAFYKYIKNLLSK-I--DGADYLL--DKIEREDFLRKORTFDNGSIPHQIHLQEM | 420 |
| WP_049538452 | 358 | --DGYAG YVDG K TTQEAFYKYIKNLLSK-F--EGADYFL--EKIEREDFLRKORTFDNGSIPHQIHLQEM | 420 |
| WP_049549711 | 358 | --DGYAG YIDG K TTQEAFYKYIKNLLSK-F--EGTDYFL--EKIEREDFLRKORTFDNGSIPHQIHLQEM | 420 |
| WP_007896501 | 359 | --NGYAG YIEG K VSQEDFYRYIKPILSR-L--KGGDEFLa--KIDRDDFLRKORTFDNGSIPHQIHLKEL | 421 |
| EFR44625 | 311 | --NGYAG YIEG K VSQEDFYRYIKPILSR-L--KGGDEFLa--KIDRDDFLRKORTFDNGSIPHQIHLKEL | 373 |
| WP_002897477 | 357 | --DGYAG YIDG K TTQEAFYKYIKNLLSK-F--EGADYFL--DKIEREDFLRKORTFDNGSIPHQIHLQEM | 419 |
| WP_002906454 | 357 | --DGYAG FIDG K TTQEAFYKYIKNLLSK-L--EGADYFL--NKIEREDFLRKORTFDNGSIPHQIHLQEM | 419 |
| WP_009729476 | 358 | --DGYAG YIDG K TTQETFYKYIKNLLSK-F--EGADYFL--DKIEREDFLRKORTFDNGSIPHQIHLQEM | 420 |
| CQR24647 | 358 | --NGYAG YIDG K TNQEDFYKYLKNLLQK-V--DGGDYFI--EKIEREDFLRKORTFDNGSIPHQVHLDEM | 420 |
| WP_000066813 | 358 | --DGYAG YIDG K TTQEAFYKYIKNLLSK-F--EGADYFL--DKIEREDFLKKORTFDNGSIPHQIHLQEM | 420 |
| WP_009754323 | 358 | --DGYAG YIDG K TTQEAFYKYIKNLLSK-F--EGADYFL--DKIEREDFLRKORTEDNGSIPHQIHLQEM | 420 |
| WP_044674937 | 357 | --DGYAG YIEG K TTQENFYRFIKKAIEK-I--EGSDYFI--DKIDREDFLRKORTFDNGSIPHOIHLQEM | 419 |
| WP_044676715 | 357 | --DGYAG YIEG K TTQENFYRFIKKAIEK-I--EGSNYFI--DKIDREDFLRKORTFDNGSIPHQIHLQEM | 419 |
| WP_044680361 | 357 | --DGYAG YIEG K TTQENFYRFIKKAIEK-I--EGSNYFI--DKIDREDFLRKORTFDNGSIPHQIHLQEM | 419 |
| WP_044681799 | 357 | --DGYAG YIEG K TTQENFYRFIKKAIEK-I--EGSDYFI--DKIDREDFLRKORTFDNGSIPHQIHLQEM | 419 |
| WP_049533112 | 357 | --KGYAG YIEN G VKQDEFYKYLKGILLQ-I--NGSGDFL--DKIDREDFLRKORTFDNGSIPHQIHLQEM | 419 |
| WP_029090905 | 316 | fyTDYIG YEES K SKEERLFKHIELLLAKENVITTVEHALleKNITFASLLPLORSSRNAVIPYQVHEKEL | 403 |
| WP_006506696 | 361 | ksKGYYN YINR K APVDEFYKYVKKCIEK-VdtPEAKQILn--DIELENFLLKONSRINGSVPYQMQLDEM | 429 |
| AIT42264 | 357 | --NGYAG YIDG G ASQEEFYKFIKPILEK-M--DGTEELLv--KLNREDLLRKORTFDNGSIPHQIHLGEL | 419 |
| WP_034440723 | 363 | --NGYAG YIDG K TSQEDFYKFVKAQLKG---eENGEYFL--EAIENENFLRKORSFYNGVIPYQIHLQEL | 425 |
| AKQ21048 | 357 | --NGYAG YIDG G ASQEEFYKFIKPILEK-M--DGTEELLv--KLNREDLLRKORTFDNGSIPHQIHLGEL | 419 |
| WP_004636532 | 356 | --NGYAG YIDG K TNQEDFYKYIEKVMKT-IksDKKDYFL--DKIDREVFLRKORSFYNSVIPHQIHLQEM | 420 |
| WP_002364836 | 363 | --DGYAG YIAH A VSQLKFYQYVKKIIQD-I--AGAEYFL--EKIAQENFLRKORTFDNGVIPHQIHLAEL | 427 |
| WP_016631044 | 314 | --DGYAG YIAH A VSQLKFYQYVKKIIQD-I--AGAEYFL--EKIAQENFLRKORTFDNGVIPHQIHLAEL | 378 |
| EMS75795 | 100 | --NGYAG YIDG K TTQEDFYKFLKKELNG-I--AGSERFM--EKVDQENFLLKORTTANGVIPHQVHLTEL | 162 |
| WP_002373311 | 363 | --DGYAG YIAH A VSQLKFYQYVKKIIQD-I--AGAEYFL--EKIAQENFLRKORTFDNGVIPHQIHLAEL | 427 |
| WP_002378009 | 363 | --DGYAG YITH A VSQLKFYQYVKKIIQD-I--AGAEYFL--EKIAQENFLRKORTFDNGVIPHQIHLAEL | 427 |
| WP_002407324 | 363 | --DGYAG YITH A VSQLKFYQYVKKIIQD-I--AGAEYFL--EKIAQENFLRKORTFDNGVIPHQIHLAEL | 427 |
| WP_002413717 | 363 | --DGYAG YIAH A VSQLKFYQYVKKIIQD-I--AGAEYFL--EKIAQENFLRKORTFDNGVIPHQIHLAEL | 427 |
| WP_010775580 | 365 | --DGYAG YIAH A VSQLKFYQYVKKIIQD-I--AGAEYFL--EKIAQENFLRKORTFDNGVIPHQIHLAEL | 429 |
| WP_010818269 | 363 | --DGYAG YIAH A VSQLKFYQYVKKIIQD-I--AGAEYFL--EKIAQENFLRKORTFDNGVIPHQIHLAEL | 427 |
| WP_010824395 | 363 | --DGYAG YITH A VSQLKFYQYVKKIIQD-I--AGAEYFL--EKIAQENFLRKORTFDNGVIPHQIHLAEL | 427 |
| WP_016622645 | 363 | --DGYAG YIAH A VSQLKFYQYVKKIIQD-I--AGAEYFL--EKIAQENFLRKORTFDNGVIPHQIHLAEL | 427 |
| WP_033624816 | 363 | --DGYAG YIAH A VSQLKFYQYVKKIIQD-I--AGAEYFL--EKIAQENFLRKORTFDNGVIPHQIHLAEL | 427 |
| WP_033625576 | 363 | --DGYAG YIAH A VSQLKFYQYVKKIIQD-I--AGAEYFL--EKIAQENFLRKORTFDNGVIPHQIHLAEL | 427 |
| WP_033789179 | 363 | --DGYAG YIAH A VSQLKFYQYVKKIIQD-I--AGAEYFL--EKIAQENFLRKORTFDNGVIPHQIHLAEL | 427 |
| WP_002310644 | 361 | --NGYAG YIEG H ATQEDFYKFVKKELTG-I--RGSEVFL--TKIEQENFLRKORTFDNGVIPHQIHLTEL | 423 |
| WP_002312694 | 362 | --NGYAG YIEG H ATQEAFYKFVKKELTG-I--RGSEVFL--TKIEQENFLRKORTFDNGVIPHQIHLSEL | 424 |
| WP_002314015 | 362 | --NGYAG YIEG H ATQEDFYKFVKKELTG-I--RGSEVFL--TKIEQENFLRKORTFDNGVIPHQIHLTEL | 424 |
| WP_002320716 | 362 | --NGYAG YIEG H ATQEDFYKFVKKELTG-I--RGSEVFL--TKIEQENFLRKORTFDNGVIPHQIHLTEL | 424 |
| WP_002330729 | 361 | --NGYAG YIEG H ATQEDFYKFVKKELTG-I--RGSEVFL--TKIEQENFLRKORTFDNGVIPHQIHLTEL | 423 |
| WP_002335161 | 362 | --NGYAG YIEG H ATQEDFYKFVKKELTG-I--RGSEVFL--TKIEQENFLRKQRTFDNGVIPHQIHLTEL | 424 |
| WP_002345439 | 362 | --NGYAG YIEG H ATQEDFYKFVKKELTG-I--RGSEVFL--TKIEQENFLRKORTFDNGVIPHQIHLTEL | 424 |
| WP_034867970 | 355 | --NGYAG YIKG K TTQEEFYKFVKKELSG-V--VGSEPFL--EKIDQETFLLKORTYTNGVIPHQVHLIEL | 417 |
| WP_047937432 | 362 | --NGYAG YIEG H ATQEDFYKFVKKELTG-I--RGSEVFL--TKIEQENFLRKORTFDNGVIPHQIHLTEL | 424 |
| WP_010720994 | 355 | --NGYAG YIKG K TTQEEFYKFVKKELSG-V--VGSEPFL--EKIDQETFLLKORTYTNGVIPHQVHLIEL | 417 |
| WP_010737004 | 355 | --NGYAG YIKG K TTQEEFYKFVKKELSG-V--VGSEPFL--EKIDQETFLLKORTYTNGVIPHQVHLIEL | 417 |
| WP_034700478 | 355 | --NGYAG YIKG K TTQEEFYKFVKKELSG-V--VGSEPFL--EKIDQETFLLKORTYTNGVIPHQVHLIEL | 417 |
| WP_007209003 | 359 | --NGYAG YIDG K TKEEEFYKYLKTTLVQ---kSGYQYFI--EKIEQENFLRKORIYDNGVIPHQVHAEEL | 421 |
| WP_023519017 | 355 | --NGYAG YVKG K ATQEDFYKFLRTELAG-L--EESQSIM--EKIDLEIYLLKORTFANGVIPHQIHLVEM | 417 |
| WP_010770040 | 358 | --SGYAG YVEN S VTQAEFYKYIKKAIEK-V--PGAEYFL--EKIEQETFLDKORTENNGVIPHQIHLEEL | 422 |
| WP_048604708 | 354 | --DGYAG YIDN S TSQEKFYKYITNLIEK-I--DGAEYFL--KKIENEDFLRKORTFDNGIIPHQIHLEEL | 418 |
| WP_010750235 | 355 | --DGYAG YIDG K TTQADFYKFLKKELTG-V--PGSEPML--AKIDQENFLLKORTPINGVIPHQVHLTEF | 417 |
| AII16583 | 396 | --NGYAG YIDG G ASQEEFYKFIKPILEK-M--DGTEELLv--KLNREDLLRKORTEDNGSIPHQIHLGEL | 458 |
| WP_029073316 | 367 | kkNNYCN YINH K TPVDEFYKYIKKLIEK-IddPDVKTILn--KIELESFMLKONSRINGAVPYQMQLDEL | 435 |
| WP_031589969 | 367 | kkNNYCN YINH K TPVDEFYKYIKKLIEK-IddPDVKTILn--KIELESFMLKONSRINGAVPYQMQLDEL | 435 |
| KDA45870 | 354 | -iSGYAG YIDG K VSEEDFYKYTKKTLKG-I--PETEEILq--KIDANNYLRKORTFDNGAIPHQVHLKEL | 417 |
| WP_039099354 | 360 | ------- YVDG K -SKEDFYGDITKALKNnPdhPIVSEIKk--LIELDQFMPKORTKDNGAIPHQLHQQEL | 425 |
| AKP02966 | 349 | --QAYDD YINK K ---KELYTSLKKFLKVaLp-TNLAKEAe-EKISKGTYLVKPRNSENGVVPYQLNKIEM | 415 |
| WP_010991369 | 363 | --HGYAG YIDG - TKQADFYKYMKMTLEN-I--EGADYFI--AKIEKENFLRKORTFDNGAIPHOLHLEEL | 425 |
| WP_033838504 | 363 | --HGYAG YIDG - TKQADFYKYMKMTLEN-I--EGADYFI--AKIEKENFLRKORTFDNGAIPHOLHLEEL | 425 |
| EHN60060 | 366 | --HGYAG YIDG - TKQADFYKYMKMTLEN-I--EGADYFI--AKIEKENFLRKORTFDNGAIPHOLHLEEL | 428 |
| EFR89594 | 132 | --HGYAG YIDG - TKQADFYKYMKTTLEN-I--EGADYFI--AKIEKENFLRKORTFDNGAIPHOLHLEEL | 194 |
| WP_038409211 | 363 | --DGYAG YIDG - TTQEKFYKYMKKMLAN-I--DGADYFI--DQIEEENFLRKQRTFDNGTIPHQLHLEEL | 425 |
| EFR95520 | 1 | ------- ---- - ---------MKKMLAN-I--DGADYFI--DQIEEENFLRKORTFDNGTIPHQLHLEEL | 44 |
| WP_003723650 | 363 | --DGYAG YIDG - TKQVDFYKYLKTILEN-I--EGSDYFI--AKIEEENFLRKORTFDNGAIPHOLHLEEL | 425 |
| WP_003727705 | 363 | --DGYAG YIDG - TKQVDFYKYLKTTLEN-V--EGADYFI--TKIEEENFLRKORTFDNGVIPHQLHLEEL | 425 |
| WP_003730785 | 363 | --DGYAG YIDG - TKQVDFYKYLKTTLEN-V--EGADYFI--TKIEEENFLRKORTFDNGVIPHQLHLEEL | 425 |
| WP_003733029 | 363 | --HGYAG YISG - TKQADFYKYMKATLEK-I--EGADYFI--AKIEEENFLRKORTFDNGVIPHQLHLEEL | 425 |
| WP_003739838 | 363 | --DGYAG YIDG - TKQVDFYKYLKTLLEN-I--EGADYFI--AKIEEENFLRKORTEDNGAIPHOLHLEEL | 425 |
| WP_014601172 | 363 | --DGYAG YIDG - TKQVDFYKYLKTILEN-I--EGADYFI--AKIEEENFLRKORTFDNGAIPHOLHLEEL | 425 |
| WP_023548323 | 363 | --DGYAG YIDG - TKQVDFYKYLKTTLEN-V--EGADYFI--TKIEEENFLRKORTFDNGVIPHQLHLEEL | 425 |
| WP_031665337 | 363 | --DGYAG YIDG - TKQVDFYKYLKTILEN-I--EGSDYFI--AKIEEENFLRKORTFDNGAIPHOLHLEEL | 425 |
| WP_031669209 | 363 | --HGYAG YISG - TKQADFYKYMKATLEK-I--EGADYFI--AKIEEENFLRKORTFDNGVIPHOLHLEEL | 425 |
| WP_033920898 | 363 | --DGYAG YIDG - TKQVDFYKYLKTILEN-I--EGADYFI--AKIEEENFLRKORTFDNGAIPHOLHLEEL | 425 |
| AKI42028 | 366 | --DGYAG YIDG - TKQVDFYKYLKTILEN-I--EGADYFI--AKIEEENFLRKORTFDNGAIPHOLHLEEL | 428 |
| AKI50529 | 366 | --DGYAG YIDG - TKQVDFYKYLKTILEN-I--EGADYFI--AKIEEENFLRKORTFDNGAIPHOLHLEEL | 428 |
| EFR83390 | | ------- ---- - ---------------------------------------------------------- | |
| WP_046323366 | 363 | --DGYAG YIEG - TKQEAFYKYMKKMLEH-V--EGADYFI--NQIEEENFLRKORTEDNGAIPHOLHLEEL | 425 |
| AKE81011 | 373 | --NGYAG YIDG G ASQEEFYKFIKPILEK-M--DGTEELLv--KLNREDLLRKORTFDNGSIPHQIHLGEL | 435 |
| CUO82355 | 365 | kvKGYYN YINR K APVDEFYKFVKKCIEK-VdtPEAKQILh--DIELENFLLKONSRINGSVPYQMQLDEM | 433 |
| WP_033162887 | 366 | k1HNYLG YIKY D TPVEEFYKYIKGLLAK-VdtDEAREILe--RIDLEKFMLKONSRINGSIPYQMQKDEM | 434 |
| AGZ01981 | 390 | --NGYAG YIDG G ASQEEFYKFIKPILEK-M--DGTEELLv--KLNREDLLRKORTFDNGSIPHQIHLGEL | 452 |
| AKA60242 | 357 | --NGYAG YIDG G ASQEEFYKFIKPILEK-M--DGTEELLv--KLNREDLLRKORTFDNGSIPHQIHLGEL | 419 |
| AKS40380 | 357 | --NGYAG YIDG G ASQEEFYKFIKPILEK-M--DGTEELLv--KLNREDLLRKORTFDNGSIPHQIHLGEL | 419 |
| 4UN5_B | 361 | --NGYAG YIDG G ASQEEFYKFIKPILEK-M--DGTEELLv--KLNREDLLRKORTFDNGSIPHQIHLGEL | 423 |
| WP_010922251 | 420 | 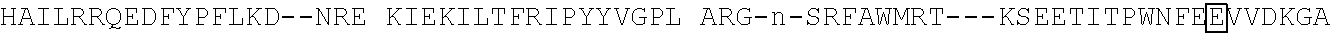 | |
| WP_039695303 | 423 | HAILRRQGDYYPFLKE--KQD RIEKILTFRIPYYVGPL VRKD--SRFAWAEY---RSDEKITPWNEDKVIDKEK | 489 |
| WP_045635197 | 420 | NAILRRQGEYYPFLKD--NKE KIEKILTFRIPYYVGPL ARGN--RDFAWLTR---NSDEAIRPWNFEEIVDKAS | 486 |
| 5AXW_A | 184 | KQLLKVOKAYHOLDQSfi--D TYIDLLETRRTYYEGPG ---Eg-SPFGWKDI---------------------- | 229 |
| WP_009880683 | 104 | HAILRRQEDFYPFLKD--NRE KIEKILTFRIPYYVGPL ARG-n-SRFAWMTR---KSEETITPWNFEEVVDKGA | 170 |
| WP_010922251 | 420 | HAILRRQEDFYPFLKD--NRE KIEKILTFRIPYYVGPL ARG-n-SRFAWMTR---KSEETITPWNFEEVVDKGA | 486 |
| WP_011054416 | 420 | HAILRRQEDFYPFLKD--NRE KIEKILTFRIPYYVGPL ARG-n-SRFAWMTR---KSEETITPWNFEEVVDKGA | 486 |
| WP_011284745 | 420 | HAILRRQEDFYPFLKD--NRE KIEKILTFRIPYYVGPL ARG-n-SRFAWMTR---KSEETITPWNFEEVVDKGA | 486 |
| WP_011285506 | 420 | HAILRRQEDFYPFLKD--NRE KIEKILTFRIPYYVGPL ARG-n-SRFAWMTR---KSEETITPWNFEEVVDKGA | 486 |
| WP_011527619 | 420 | HAILRRQEDFYPFLKD--NRE KIEKILTFRIPYYVGPL ARG-n-SRFAWMTR---KSEETITPWNFEEVVDKGA | 486 |
| WP_012560673 | 420 | HAILRRQEDFYPFLKD--NRE KIEKILTFRIPYYVGPL ARG-n-SRFAWMTR---KSEETITPWNFEEVVDKGA | 486 |
| WP_014407541 | 420 | HAILRRQEDFYPFLKD--NRE KIEKILTFRIPYYVGPL ARG-n-SRFAWMTR---KSEETITPWNFEEVVDKGA | 486 |
| WP_020905136 | 420 | HAILRRQEDFYPFLKD--NRE KIEKILTFRIPYYVGPL ARG-n-SRFAWMTR---KSEETITPWNFEEVVDKGA | 486 |
| WP_023080005 | 420 | HAILRRQEDFYPFLKD--NRE KIEKILTFRIPYYVGPL ARG-n-SRFAWMTR---KSEETITPWNFEEVVDKGA | 486 |
| WP_023610282 | 420 | HAILRRQEDFYPFLKD--NRE KIEKILTFRIPYYVGPL ARG-n-SRFAWMTR---KSEETITPWNFEEVVDKGA | 486 |
| WP_030125963 | 420 | HAILRRQEDFYPFLKD--NRE KIEKILTFRIPYYVGPL ARG-n-SRFAWMTR---KSEETITPWNFEEVVDKGA | 486 |
| WP_030126706 | 420 | HAILRRQEDFYPFLKD--NRE KIEKILTFRIPYYVGPL ARG-n-SRFAWMTR---KSEETITPWNFEEVVDKGA | 486 |
| WP_031488318 | 420 | HAILRRQEDFYPFLKD--NRE KIEKILTFRIPYYVGPL ARG-n-SRFAWMTR---KSEETITPWNFEEVVDKGA | 486 |
| WP_032460140 | 420 | HAILRRQEDFYPFLKD--NRE KIEKILTFRIPYYVGPL ARG-n-SRFAWMTR---KSEETITPWNFEEVVDKGA | 486 |
| WP_032461047 | 420 | HAILRRQEDFYPFLKD--NRE KIEKILTFRIPYYVGPL ARG-n-SRFAWMTR---KSEETITPWNFEEVVDKGA | 486 |
| WP_032462016 | 420 | HAILRRQEDFYPFLKD--NRE KIEKILTFRIPYYVGPL ARG-n-SRFAWMTR---KSEETITPWNFEEVVDKGA | 486 |
| WP_032462936 | 420 | HAILRRQEDFYPFLKD--NRE KIEKILTFRIPYYVGPL ARG-n-SRFAWMTR---KSEETITPWNFEEVVDKGA | 486 |
| WP_032464890 | 420 | HAILRRQEDFYPFLKD--NRE KIEKILTFRIPYYVGPL ARG-n-SRFAWMTR---KSEETITPWNFEEVVDKGA | 486 |
| WP_033888930 | 245 | HAILRRQEDFYPFLKD--NRE KIEKILTFRIPYYVGPL ARG-n-SRFAWMTR---KSEETITPWNFEEVVDKGA | 311 |
| WP_038431314 | 420 | HAILRRQEDFYPFLKD--NRE KIEKILTFRIPYYVGPL ARG-n-SRFAWMTR---KSEETITPWNFEEVVDKGA | 486 |
| WP_038432938 | 420 | HAILRRQEDFYPFLKD--NRE KIEKILTFRIPYYVGPL ARG-n-SRFAWMTR---KSEETITPWNFEEVVDKGA | 486 |
| WP_038434062 | 420 | HAILRRQEDFYPFLKD--NRE KIEKILTFRIPYYVGPL ARG-n-SRFAWMTR---KSEETITPWNFEEVVDKGA | 486 |
| BAQ51233 | 331 | HAILRRQEDFYPFLKD--NRE KIEKILTFRIPYYVGPL ARG-n-SRFAWMTR---KSEETITPWNFEEVVDKGA | 397 |
| KGE60162 | | --------------------- ----------------- ------------------------------------ | |
| KGE60856 | | --------------------- ----------------- ------------------------------------ | |
| WP_002989955 | 420 | HAILRRQEDFYPFLKD--NRE KIEKILTFRIPYYVGPL ARG-n-SRFAWMTR---KSEETITPWNFEEVVDKGA | 486 |
| WP_003030002 | 420 | HAILRRQEEHYPFLKE--NQD KIEKILTFRIPYYVGPL ARKG--SRFAWAEY---KADEKITPWNEDDILDKEK | 486 |
| WP_003065552 | 423 | HAILRRQGDYYPFLKE--NQD RIEKILTFRIPYYVGPL ARKD--SRFSWAEY---HSDEKITPWNEDKVIDKEK | 489 |
| WP_001040076 | 421 | RAIIRRQSEYYPFLKE--NLD RIEKILTFRIPYYVGPL AREK--SDFAWMTR---KTDDSIRPWNFEDLVDKEK | 487 |
| WP_001040078 | 421 | KAIIRRQSEYYPFLKE--NQD RIEKILTFRIPYYIGPL AREK--SDFAWMTR---KTDDSIRPWNFEDLVDKEK | 487 |
| WP_001040080 | 421 | KAIIRRQSEYYPFLKE--NQD RIEKILTFRIPYYIGPL AREK--SDFAWMTR---KTDDSIRPWNFEDLVDKEK | 487 |
| WP_001040081 | 421 | KAIIRRQSEYYPFLKE--NQD RIEKILTFRIPYYIGPL AREK--SDFAWMTR---KTDDSIRPWNFEDLVDKEK | 487 |
| WP_001040083 | 421 | KAIIRRQSEYYPFLKE--NQD RIEKILTFRIPYYIGPL AREK--SDFAWMTR---KTDDSIRPWNFEDLVDKEK | 487 |
| WP_001040085 | 421 | KAIIRRQSEYYPFLKE--NQD RIEKILTFRIPYYIGPL AREK--SDFAWMTR---KTDDSIRPWNFEDLVDKEK | 487 |
| WP_001040087 | 421 | KAIIRRQSEYYPFLKE--NQD RIEKILTFRIPYYIGPL AREK--SDFAWMTR---KTDDSIRPWNFEDLVDKEK | 487 |
| WP_001040088 | 421 | KAIIRROSEYYPFLKE--NQD RIEKILTFRIPYYIGPL AREK--SDFAWMTR---KTDDSIRPWNFEDLVDKEK | 487 |
| WP_001040089 | 421 | KAIIRRQSEYYPFLKE--NQD RIEKILTFRIPYYIGPL AREK--SDFAWMTR---KTDDSIRPWNFEDLVDKEK | 487 |
| WP_001040090 | 421 | KAIIRRQSEYYPFLKE--NQD RIEKILTFRIPYYIGPL AREK--SDFAWMTR---KTDDSIRPWNFEDLVDKEK | 487 |
| WP_001040091 | 421 | KAIIRRQSEYYPFLKE--NQD RIEKILTFRIPYYIGPL AREK--SDFAWMTR---KTDDSIRPWNFEDLVDKEK | 487 |
| WP_001040092 | 421 | KAIIRRQSEYYPFLKE--NQD KIEKILTFRIPYYVGPL ARGN--SDFAWMTR---KTDDSIRPWNFEDLVDKEK | 487 |
| WP_001040094 | 421 | RAIIRROSEYYPFLKE--NLD RIEKILTFRIPYYVGPL AREK--SDFAWMTR---KTDDSIRPWNFEDLVDKEK | 487 |
| WP_001040095 | 421 | RAIIRRQSEYYPFLKE--NLD RIEKILTFRIPYYVGPL AREK--SDFAWMTR---KTDDSIRPWNFEDLVDKEK | 487 |
| WP_001040096 | 421 | RAIIRRQSEYYPFLKE--NLD RIEKILTFRIPYYVGPL AREK--SDFAWMTR---KTDDSIRPWNFEDLVDKEK | 487 |
| WP_001040097 | 421 | RAIIRRQSEYYPFLKE--NLD RIEKILTFRIPYYVGPL AREK--SDFAWMTR---KTDDSIRPWNFEDLVDKEK | 487 |
| WP_001040098 | 421 | RAIIRRQSEYYPFLKE--NLD RIEKILTFRIPYYVGPL AREK--SDFAWMTR---KTDDSIRPWNFEDLVDKEK | 487 |
| WP_001040099 | 421 | RAIIRRQSEYYPFLKE--NLD RIEKILTFRIPYYVGPL AREK--SDFAWMTR---KTDDSIRPWNFEDLVDKEK | 487 |
| WP_001040100 | 421 | RAIIRRQSEYYPLLKE--NLD RIEKILTFRIPYYVGPL AREK--SDFAWMTR---KTDDSIRPWNFEDLVDKEK | 487 |
| WP_001040104 | 421 | KAIIRROSEYYPFLKE--NQD RIEKILTFRIPYYIGPL AREK--SDFAWMTR---KTDDSIRPWNFEDLVDKEK | 487 |
| WP_001040105 | 421 | KAIIRROSEYYPFLKE--NQD RIEKILTFRIPYYIGPL AREK--SDFAWMTR---KTDDSIRPWNFEDLVDKEK | 487 |
| WP_001040106 | 421 | KAIIRROSEYYPFLKE--NLD RIEKILTFRIPYYVGPL AREK--SDFAWMTR---KTDDSIRPWNFEELVDKEA | 487 |
| WP_001040107 | 421 | KAIIRROSEYYPFLKE--NLD RIEKILTFRIPYYVGPL AREK--SDFAWMTR---KTDDSIRPWNFEELVDKEA | 487 |
| WP_001040108 | 421 | KAIIRROSEYYPFLKE--NLD RIEKILTFRIPYYVGPL AREK--SDFAWMTR---KTDDSIRPWNFEELVDKEA | 487 |
| WP_001040109 | 421 | KAIIRROSEYYPFLKE--NLD RIEKILTFRIPYYVGPL AREK--SDFAWMTR---KTDDSIRPWNFEELVDKEA | 487 |
| WP_001040110 | 421 | KAIIRROSEYYPFLKE--NLD RIEKILTFRIPYYVGPL AREK--SDFAWMTR---KTDDSIRPWNFEELVDKEA | 487 |
| WP_015058523 | 421 | KAIIRROSEYYPFLKE--NQD KIEKILTFRIPYYVGPL ARGN--SDFAWMTR---KTDDSIRPWNFEDLVDKEK | 487 |
| WP_017643650 | 421 | RAIIRROSEYYPFLKE--NLD RIEKILTFRIPYYVGPL AREK--SDFAWMTR---KTDDSIRPWNFEDLVDKEK | 487 |
| WP_017647151 | 421 | KAIIRRQSEYYPFLKE--NLD RIEKILTFRIPYYVGPL AREK--SDFAWMTR---KTDDSIRPWNFEELVDKEA | 487 |
| WP_017648376 | 421 | KAIIRROSEYYPFLKE--NLD RIEKILTFRIPYYVGPL AREK--SDFAWMTR---KTDDSIRPWNFEELVDKEA | 487 |
| WP_017649527 | 421 | KAIIRRQSEYYPFLKE--NQD RIEKILTFRIPYYIGPL AREK--SDFAWMTR---KTDDSIRPWNFEDLVDKEK | 487 |
| WP_017771611 | 421 | KAIIRROSEYYPFLKE--NLD RIEKILTFRIPYYVGPL AREK--SDFAWMTR---KTDDSIRPWNFEELVDKEA | 487 |
| WP_017771984 | 421 | KAIIRROSEYYPFLKE--NQD RIEKILTFRIPYYIGPL AREK--SDFAWMTR---KTDDSIRPWNFEDLVDKEK | 487 |
| CFQ25032 | 421 | KAIIRRQSEYYPFLKE--NQD RIEKILTFRIPYYIGPL AREK--SDFAWMTR---KTDDSIRPWNFEDLVDKEK | 487 |
| CFV16040 | 421 | KAIIRRQSEYYPFLKE--NQD RIEKILTFRIPYYIGPL AREK--SDFAWMTR---KTDDSIRPWNFEDLVDKEK | 487 |
| KLJ37842 | 421 | KAIIRRQSEYYPFLKE--NQD RIEKILTFRIPYYIGPL AREK--SDFAWMTR---KTDDSIRPWNFEDLVDKEK | 487 |
| KLJ72361 | 421 | KAIIRRQSEYYPFLKE--NQD RIEKILTFRIPYYIGPL AREK--SDFAWMTR---KTDDSIRPWNFEDLVDKEK | 487 |
| KLL20707 | 421 | KAIIRRQSEYYPFLKE--NQD RIEKILTFRIPYYIGPL AREK--SDFAWMTR---KTDDSIRPWNFEDLVDKEK | 487 |
| KLL42645 | 421 | KAIIRRQSEYYPFLKE--NLD RIEKILTFRIPYYVGPL AREK--SDFAWMTR---KTDDSIRPWNFEELVDKEA | 487 |
| WP_047207273 | 421 | KAIIRROSEYYPFLKE--NQD RIEKILTFRIPYYIGPL AREK--SDFAWMTR---KIDDSIRPWNFEDLVDKEK | 487 |
| WP_047209694 | 421 | RAIIRRQSEYYPFLKE--NLD RIEKILTFRIPYYVGPL AREK--SDFAWMTR---KIDDSIRPWNFEDLVDKEK | 487 |
| WP_050198062 | 421 | KAIIRROSEYYPFLKE--NQD RIEKILTFRIPYYIGPL AREK--SDFAWMTR---KIDDSIRPWNFEDLVDKEK | 487 |
| WP_050201642 | 421 | KAIIRRQSEYYPFLKE--NQD RIEKILTFRIPYYIGPL AREK--SDFAWMTR---KTDDSIRPWNFEDLVDKEK | 487 |
| WP_050204027 | 421 | KAIIRRQSEYYPFLKE--NLD RIEKILTFRIPYYVGPL AREK--SDFAWMTR---KIDDSIRPWNFEELVDKEA | 487 |
| WP_050881965 | 421 | KAIIRROSEYYPFLKE--NQD RIEKILTFRIPYYIGPL AREK--SDFAWMTR---KTDDSIRPWNFEDLVDKEK | 487 |
| WP_050886065 | 421 | KDIIRRQSEYYPFLKE--NQD RIEKILTFRIPYYIGPL AREK--SDFAWMTR---KTDDSIRPWNFEDLVDKEK | 487 |
| AHN30376 | 421 | KAIIRRQSEYYPFLKE--NQD KIEKILTFRIPYYVGPL ARGN--SDFAWMTR---KTDDSIRPWNFEDLVDKEK | 487 |
| EAO78426 | 421 | KAIIRRQSEYYPFLKE--NQD RIEKILTFRIPYYIGPL AREK--SDFAWMTR---KTDDSIRPWNFEDLVDKEK | 487 |
| CCW42055 | 421 | RAIIRROSEYYPFLKE--NLD RIEKILTFRIPYYVGPL AREK--SDFAWMTR---KTDDSIRPWNFEELVDKEA | 487 |
| WP_003041502 | 420 | HAILRRQGEHYPFLKE--NQD KIEKILTFRIPYYVGPL ARKG--SRFAWAEY---KADEKITPWNEDDILDKEK | 486 |
| WP_037593752 | 421 | HAILRRQGEHYPFLKE--NQD KIEKILTFRIPYYVGPL ARKG--SRFAWAEY---KADEKITPWNEDDILDKEK | 487 |
| WP_049516684 | 421 | HAILRRQGEHYPFLKE--NQD KIEKILTFRIPYYVGPL ARKG--SRFAWAEY---KADEKITPWNEDDILDKEK | 487 |
| GAD46167 | 420 | HAILRRQGEHYPFLKE--NQD KIEKILTFRIPYYVGPL ARKG--SRFAWAEY---KADEKITPWNEDDILDKEK | 486 |
| WP_018363470 | 421 | HAILRRQGDYYPFLKE--NQE EIEKILTFRIPYYVGPL ARKD--SRFAWAEY---RSDEKITPWNEDKVIDKEK | 487 |
| WP_003043819 | 430 | HAILRRQEEFYPFLKE--NRE KIEKILTFRIPYYVGPL ARG-n-SRFAWLTR---KSEEAITPWNFEEVVDKGA | 496 |
| WP_006269658 | 420 | HAILRRQGEHYPFLKE--NQD KIEKILTFRIPYYVGPL ARKG--SRFAWAEY---KADEKITPWNEDDILDKEK | 486 |
| WP_048800889 | 420 | HAILRRQGEHYPFLKE--NQD KIEKILTFRIPYYVGPL VRKG--SRFAWAEY---KADEKITPWNEDDILDKEK | 486 |
| WP_012767106 | 420 | HAILRRQEDFYPFLKD--NRE KIEKILTFRIPYYVGPL ARG-n-SRFAWMTR---KSEETITPWNFEEVVDKGA | 486 |
| WP_014612333 | 420 | HAILRRQEDFYPFLKD--NRE KIEKILTFRIPYYVGPL ARG-n-SRFAWMTR---KSEETITPWNFEEVVDKGA | 486 |
| WP_015017095 | 420 | HAILRRQEDFYPFLKD--NRE KIEKILTFRIPYYVGPL ARG-n-SRFAWMTR---KSEETITPWNFEEVVDKGA | 486 |
| WP_015057649 | 420 | HAILRRQEDFYPFLKD--NRE KIEKILTFRIPYYVGPL ARG-n-SRFAWMTR---KSEETITPWNFEEVVDKGA | 486 |
| WP_048327215 | 420 | HAILRRQEDFYPFLKD--NRE KIEKILTFRIPYYVGPL ARG-n-SRFAWMTR---KSEETITPWNFEEVVDKGA | 486 |
| WP_049519324 | 420 | HAILRRQEDFYPFLKD--NRE KIEKILTFRIPYYVGPL ARG-n-SRFAWMTR---KSEETITPWNFEEVVDKGA | 486 |
| WP_012515931 | 420 | HAILRRQEVFYPFLKD--NRK KIESLLTFRIPYYVGPL ARG-h-SRFAWVKR---KFDGAIRPWNFEEIVDEEA | 486 |
| WP_021320964 | 420 | HAILRRQEVFYPFLKD--NRK KIESLLTFRIPYYVGPL ARG-h-SRFAWVKR---KFDGAIRPWNFEEIVDEEA | 486 |
| WP_037581760 | 420 | HAILRRQEVFYPFLKD--NRK KIESLLTFRIPYYVGPL ARG-h-SRFAWVKR---KFDGAIRPWNFEEIVDEEA | 486 |
| WP_004232481 | 420 | RTILRRQGEYYPFLKE--NQA KIEKILTFRIPYYVGPL ARKN--SRFAWAKY---HSDEPITPWNFDEVVDKEK | 486 |
| WP_009854540 | 421 | HAILRRQGDYYPFLKE--KQD RIEKILTFRIPYYVGPL VRKD--SRFAWAEY---RSDEKITPWNFDKVIDKEK | 487 |
| WP_012962174 | 421 | HAILRRQGEHYAFLKE--NQA KIEKILTFRIPYYVGPL ARKN--SRFAWAEY---HSDEKITPWNFDEIIDKEK | 487 |
| WP_039695303 | 423 | HAILRRQGDYYPFLKE--KOD RIEKILTFRIPYYVGPL VRKD--SRFAWAEY---RSDEKITPWNEDKVIDKEK | 489 |
| WP_014334983 | 420 | HSILRRQGDYYPFLKE--NQA KIEKILTFRIPYYVGPL ARKD--SRFAWANY---HSDEPITPWNFDEVVDKEK | 486 |
| WP_003099269 | 420 | KAIIRRQEKFYPFLKE--NQK KIEKLFTFKIPYYVGPL ANG-q-SSFAWLKR---QSNESITPWNFEEVVDQEA | 486 |
| AHY15608 | 420 | KAIIRRQEKFYPFLKE--NQK KIEKLFTFKIPYYVGPL ANG-q-SSFAWLKR---QSNESITPWNFEEVVDQEA | 486 |
| AHY17476 | 420 | KAIIRRQEKFYPFLKE--NQK KIEKLFTFKIPYYVGPL ANG-q-SSFAWLKR---QSNESITPWNFEEVVDQEA | 486 |
| ESR09100 | | --------------------- ----------------- ------------------------------------ | |
| AGM98575 | 420 | KAIIRRQEKFYPFLKE--NQK KIEKLFTFKIPYYVGPL ANG-q-SSFAWLKR---QSNESITPWNFEEVVDQEA | 486 |
| ALF27331 | 420 | RAIIRRQAEFYPFLAD--NQD RIEKILTFRIPYYIGPL ARGK--SDFSWLSR---KSADKITPWNFDEIVDKES | 486 |
| WP_018372492 | 433 | QAIILNQSKYYPFLAE--NKE KIEKILTFRIPYYVGPL ARGN--SSFAWLQR---KSDEAIRPWNFEQVVDMET | 499 |
| WP_045618028 | 421 | NAIIRRQGEHYPFLQE--NKE KIEKILTFRIPYYVGPL ARGN--RDFAWLTR---NSDQAIRPWNFEEIVDKAR | 487 |
| WP_045635197 | 420 | NAILRRQGEYYPFLKD--NKE KIEKILTFRIPYYVGPL ARGN--RDFAWLTR---NSDEAIRPWNFEEIVDKAS | 486 |
| WP_002263549 | 420 | RAIIRRQAEFYPFLAD--NQD RIEKLLTFRIPYYVGPL ARGK--SDFAWLSR---KSADKITPWNFDEIVDKES | 486 |
| WP_002263887 | 420 | RAIIRRQAEFYPFLAD--NQD RIEKLLTFRIPYYVGPL ARGK--SDFAWLSR---KSADKITPWNFDEIVDKES | 486 |
| WP_002264920 | 420 | HAILRRQGDYYPFLKE--NQD RIEKILTFRIPYYVGPL ARGK--SDFAWLSR---KSADKITPWNFDEIVDKES | 486 |
| WP_002269043 | 420 | RAIIRRQAEFYPFLAD--NQD RIEKILTFRIPYYVGPL ARGK--SDFAWLSR---KSADKITPWNFDEIVDKES | 486 |
| WP_002269448 | 420 | RAIIRRQAEFYPFLAD--NQD RIEKILTFRIPYYVGPL ARGK--SDFAWLSR---KSADKITPWNFDEIVDKES | 486 |
| WP_002271977 | 420 | RAIIRRQAEFYPFLAD--NQD RIEKILTFRIPYYVGPL ARGK--SDFAWLSR---KSADKITPWNFDEIVDKES | 486 |
| WP_002272766 | 420 | RAIIRRQAEFYPFLAD--NQD RIEKILTFRIPYYVGPL ARGK--SDFAWLSR---KSADKITPWNFDEIVDKES | 486 |
| WP_002273241 | 420 | RAIIRRQAEFYPFLAD--NQD RIEKILTFRIPYYVGPL ARGK--SDFAWLSR---KSADKITPWNFDEIVDKES | 486 |
| WP_002275430 | 420 | RAIIRRQAEFYPFLAD--NQD RIEKILTFRIPYYVGPL ARGK--SDFAWLSR---KSADKITPWNFDEIVDKES | 486 |
| WP_002276448 | 420 | RAIIRRQAEFYPFLAD--NQD RIEKILTFRIPYYVGPL ARGK--SDFAWLSR---KSADKITPWNFDEIVDKES | 486 |
| WP_002277050 | 420 | HAILRRQGDYYPFLKE--NQD RIEKILTFRIPYYVGPL ARKN--SRFAWAEY---HSDEAVMPWNEDQVIDKES | 486 |
| WP_002277364 | 420 | RAIIRRQAEFYPFLAD--NQD RIEKILTFRIPYYVGPL ARGK--SDFAWLSR---KSADKITPWNFDEIVDKES | 486 |
| WP_002279025 | 420 | RAIIRRQSEFYPFLAD--NQD RIEKILTFRIPYYVGPL ARGK--SDFAWLSR---KSADKITPWNFDEIVDKES | 486 |
| WP_002279859 | 420 | RAIIRRQAEFYPFLAD--NQD RIEKILTFRIPYYVGPL ARGK--SDFAWLSR---KSADKITPWNFDEIVDKES | 486 |
| WP_002280230 | 420 | RAIIRRQAEFYPFLAD--NQD RIEKILTFRIPYYVGPL ARGK--SDFAWLSR---KSADKITPWNFDEIVDKES | 486 |
| WP_002281696 | 420 | RAIIRRQAEFYPFLAD--NQD RIEKILTFRIPYYVGPL ARGK--SDFAWLSR---KSADKITPWNFDEIVDKES | 486 |
| WP_002282247 | 420 | HAILRRQGDYYPFLKE--NQD RIEKILTFRIPYYVGPL ARKN--SRFAWAEY---HSDEAVTPWNFDQVIDKES | 486 |
| WP_002282906 | 420 | RAIIRRQAEFYPFLAD--NQD RIEKLLTFRIPYYVGPL ARGK--SDFAWLSR---KSADKITPWNFDEIVDKES | 486 |
| WP_002283846 | 420 | RAIIRRQAEFYPFLAD--NQD RIEKILTFRIPYYVGPL ARGK--SDFAWLSR---KSADKITPWNFDEIVDKES | 486 |
| WP_002287255 | 420 | RAIIRRQAEFYPFLAD--NQD RIEKILTFRIPYYVGPL ARGK--SDFAWLSR---KSADKITPWNFDEIVDKES | 486 |
| WP_002288990 | 420 | RAIIRRQAEFYPFLAD--NQD RIEKILTFRIPYYVGPL ARGK--SDFAWLSR---KSADKITPWNFDEIVDKES | 486 |
| WP_002289641 | 420 | RAIIRRQAEFYPFLAD--NQD RIEKLLTFRIPYYVGPL ASGK--SDFAWLSR---KSADKITPWNFDEIVDKES | 486 |
| WP_002290427 | 420 | RAIIRRQAEFYPFLAD--NQD RIEKILTFRIPYYVGPL ARGK--SDFAWLSR---KSADKITPWNFDEIVDKES | 486 |
| WP_002295753 | 420 | RAIIRRQAEFYPFLAD--NQD RIEKILTFRIPYYVGPL ARGK--SDFAWLSR---KSADKITPWNFDEIVDKES | 486 |
| WP_002296423 | 420 | RAIIRRQAEFYPFLAD--NQD RIEKILTFRIPYYVGPL ARGK--SDFAWLSR---KSADKITPWNFDEIVDKES | 486 |
| WP_002304487 | 430 | HAILRRQGEHYPFLKE--NQD KIEKILTFRIPYYVGPL VRKG--SRFAWAEY---KADEKITPWNEDDILDKEK | 496 |
| WP_002305844 | 420 | RAIIRRQAEFYPFLAD--NQD RIEKILTFRIPYYVGPL ARGK--SDFAWLSR---KSADKITPWNFDEIVDKES | 486 |
| WP_002307203 | 420 | RAIIRRQAEFYPFLAD--NQD RIEKILTFRIPYYVGPL ARGK--SDFAWLSR---KSADKITPWNFDEIVDKES | 486 |
| WP_002310390 | 420 | RAIIRRQAEFYPFLAD--NQD RIEKILTFRIPYYVGPL ARGK--SDFAWLSR---KSADKITPWNFDEIVDKES | 486 |
| WP_002352408 | 420 | RAIIRRQAEFYPFLAD--NQD RIEKILTFRIPYYVGPL ARGK--SDFAWLSR---KSADKITPWNFDEIVDKES | 486 |
| WP_012997688 | 420 | RAIIRRQAEFYPFLAD--NQD RIEKILTFRIPYYVGPL ARGK--SDFAWLSR---KSADKITPWNFDEIVDKES | 486 |
| WP_014677909 | 420 | RAIIRRQAEFYPFLAD--NQD RIEKILTFRIPYYVGPL ARGK--SDFAWLSR---KSADKITPWNFDEIVDKES | 486 |
| WP_019312892 | 420 | RAIIRRQAEFYPFLAD--NQD RIEKILTFRIPYYVGPL ARGK--SDFAWLSR---KSADKITPWNFDEIVDKES | 486 |
| WP_019313659 | 420 | RAIIRRQAEFYPFLAD--NQD RIEKLLTFRIPYYVGPL ARGK--SDFAWLSR---KSADKITPWNFDEIVDKES | 486 |
| WP_019314093 | 420 | RAIIRRQAEFYPFLAD--NQD RIEKILTFRIPYYVGPL ARGK--SDFAWLSR---KSADKITPWNFDEIVDKES | 486 |
| WP_019315370 | 420 | RAIIRRQAEFYPFLAD--NQD RIEKILTFRIPYYVGPL ARGK--SDFAWLSR---KSADKITPWNFDEIVDKES | 486 |
| WP_019803776 | 420 | RAIIRRQAEFYPFLAD--NQD RIEKLLTFRIPYYVGPL ARGK--SDFAWLSR---KSADKITPWNFDEIVDKES | 486 |
| WP_019805234 | 420 | RAIIRRQAEFYPFLAD--NQD RIEKILTFRIPYYVGPL ARGK--SDFAWLSR---KSADKITPWNFDEIVDKES | 486 |
| WP_024783594 | 420 | RAIIRRQAEFYPFLAD--NQD RIEKLLTFRIPYYVGPL ARGK--SDFAWLSR---KSADKITPWNFDEIVDKES | 486 |
| WP_024784288 | 420 | HAILRRQGDYYPFLKE--NQD RIEKILTFRIPYYVGPL ARKN--SRFAWAEY---HSDEAVTPWNEDQVIDKES | 486 |
| WP_024784666 | 420 | RAIIRRQAEFYPFLAD--NQD RIEKILTFRIPYYVGPL ARGK--SDFAWLSR---KSADKITPWNFDEIVDKES | 486 |
| WP_024784894 | 420 | RAIIRRQAEFYPFLAD--NQD RIEKLLTFRIPYYVGPL ASGK--SDFAWLSR---KSADKITPWNFDEIVDKES | 486 |
| WP_024786433 | 420 | HAILRRQGDYYPFLKE--NQD RIEKILTFRIPYYVGPL ARKN--SRFAWAEY---HSDEAVTPWNFDQVIDKES | 486 |
| WP_049473442 | 420 | RAIIRRQAEFYPFLAD--NQD RIEKILTFRIPYYVGPL ARGK--SDFAWLSR---KSADKITPWNFDEIVDKES | 486 |
| WP_049474547 | 420 | RAIIRRQAEFYPFLAD--NQD RIEKLLTFRIPYYVGPL ASGK--SDFAWLSR---KSADKITPWNFDEIVDKES | 486 |
| EMC03581 | 413 | RAIIRRQAEFYPFLAD--NQD RIEKILTFRIPYYVGPL ARGK--SDFAWLSR---KSADKITPWNFDEIVDKES | 479 |
| WP_000428612 | 421 | NAILRRQGEHYPFLKE--NKE KIEKILTFRIPYYVGPL ARGN--RDFAWLTR---NSDQAIRPWNFEEIVDKAS | 487 |
| WP_000428613 | 421 | NAILRRQGEHYPFLKD--NKE KIEKILTFRIPYYVGPL ARGN--RDFAWLTR---NSDEAIRPWNFEEIVDKAS | 487 |
| WP_049523028 | 420 | NAILRHQGEYYPFLKE--NKD KIEQILTFRIPYYVGPL ARGN--SDFAWLSR---NSDEAIRPWNFEEMVDKSS | 486 |
| WP_003107102 | 389 | KSIIRRQEKYYPFLKD--KQV RIEKIFTFRIPYFVGPL ANG-n-SSFAWVKR---RSNESITPWNFEEVVEQEA | 455 |
| WP_054279288 | 422 | QAILERQQAYYPFLKD--NQE KIEKILTFRIPYYIGPL ARG-n-SRFAWLTR---TSDQKITPWNFDEMVDQEA | 488 |
| WP_049531101 | 421 | NAILRRQGEHYPFLKE--NRE KIEKILTFRIPYYVGPL ARGN--RDFAWLTR---NSDQAIRPWNFEEIVDKAS | 487 |
| WP_049538452 | 421 | NAILRRQGEHYPFLKE--NKE KIEKILTFRIPYYVGPL ARGN--RDFAWLTR---NSDQAIRPWNFEEIVDKAS | 487 |
| WP_049549711 | 421 | NAILRRQGEHYPFLKE--NKE KIEKILTFRIPYYVGPL ARGN--RDFAWLTR---NSDQAIRPWNFEEIVDKAS | 487 |
| WP_007896501 | 422 | HAILRRQEKYYPFLAE--QKE KIEQLLCFRIPYYVGPL AKGGn-SSFAWLKR---RSDEPITPWNFKDVVDEEA | 489 |
| EFR44625 | 374 | HAILRRQEKYYPFLAE--QKE KIEQLLCFRIPYYVGPL AKGGn-SSFAWLKR---RSDEPITPWNFKDVVDEEA | 441 |
| WP_002897477 | 420 | NAILRRQGEHYPFLKE--NRE KIEKILTFRIPYYVGPL ARDN--RDFSWLTR---NSDEPIRPWNFEEVVDKAR | 486 |
| WP_002906454 | 420 | NAILRRQGEHYLFLKE--NRE KIEKILAFRIPYYVGPL ARGN--RDFAWLTR---NSDQAIRPWNFEEVVDKAS | 486 |
| WP_009729476 | 421 | NAILRRQGEHYPFLKE--NKE KIEKILTFRIPYYVGPL ARGN--RDFAWLTR---NSDQAIRPWNFEEIVDKAS | 487 |
| CQR24647 | 421 | KAILRRQGEFYPFLKE--NAE KIQQILTFKIPYYVGPL ARGN--SRFAWASY---NSNEKMTPWNEDNVIDKTS | 487 |
| WP_000066813 | 421 | NAIIRRQGEHYPFLQE--NKE KIEKILTFRIPYYVGPL ARGN--GDFAWLTR---NSDQAIRPWNFEEIVDQAS | 487 |
| WP_009754323 | 421 | NAILRRQGEHYPLLKE--NKE KIEKILTFRIPYYVGPL ARGN--RDFAWLTR---NSDQAIRPWNFEEIVDKAS | 487 |
| WP_044674937 | 420 | HAIIRRQAEFYPFLVE--NQD KIEKILTFRIPYYVGPL ARGK--SEFAWLNR---KSDEKIRPWNFDEMVDKET | 486 |
| WP_044676715 | 420 | HAIIRRQAEFYPFLVE--NQD KIEKILTFRIPYYVGPL ARGK--SEFAWLNR---KSDEKIRPWNFDEMVDKET | 486 |
| WP_044680361 | 420 | HAIIRRQAEFYPFLVE--NQD KIEKILTFRIPYYVGPL ARGK--SEFAWLNR---KSDEKIRPWNFDEMVDKET | 486 |
| WP_044681799 | 420 | HAIIRRQAEFYPFLVE--NQD KIEKILTFRIPYYVGPL ARGK--SEFAWLNR---KSDEKIRPWNFDEMVDKET | 486 |
| WP_049533112 | 420 | HAILRRQEEHYPFLKE--NQD KIEKILTFRIPYYVGPL ARKG--SRFAWAEY---KADEKITPWNEDDILDKEK | 486 |
| WP_029090905 | 404 | VAILENQATYYPFLLE--QKD NIHKLLTFRIPYYVGPL ADQKd-SEFAWMVR---KQAGKITPFNFEEMVDIDA | 471 |
| WP_006506696 | 430 | IKIIDNQAEYYPILKE--KRE QLLSILTFRIPYYFGPL ETSEh----AWIKRlegKENQRILPWNYQDIVDVDA | 498 |
| AIT42264 | 420 | HAILRRQEDFYPFLKD--NRE KIEKILTFRIPYYVGPL ARG-n-SRFAWMTR---KSEETITPWNFEEVVDKGA | 486 |
| WP_034440723 | 426 | TAVLDQQEKHYSFLKE--NRD KIISLLTFRIPYYVGPL AKGE--SRFAWLER--sNSEEKIKPWNEDKIVDIDK | 493 |
| AKQ21048 | 420 | HAILRRQEDFYPFLKD--NRE KIEKILTFRIPYYVGPL ARG-n-SRFAWMTR---KSEETITPWNFEEVVDKGA | 486 |
| WP_004636532 | 421 | QAILDRQSQYYPFLAE--NRD KIESLVTFRIPYYVGPL TVSDq-SEFAWMER---QSDEPIRPWNEDEIVNKER | 488 |
| WP_002364836 | 428 | QAIIHRQAAYYPFLKE--NQE KIEQLVTFRIPYYVGPL SKGDa-STFAWLKR---QSEEPIRPWNLQETVDLDQ | 495 |
| WP_016631044 | 379 | QAIIHRQAAYYPFLKE--NQE KIEQLVTFRIPYYVGPL SKGDa-STFAWLKR---QSEEPIRPWNLQETVDLDQ | 446 |
| EMS75795 | 163 | KAIIERQKPYYPSLEE--ARD KMIRLLTFRIPYYVGPL AQGEetSSFAWLER---KTPEKVTPWNATEVIDYSA | 231 |
| WP_002373311 | 428 | QAIIHRQAAYYPFLKE--NQE KIEQLVTFRIPYYVGPL SKGDa-STFAWLKR---QSEEPIRPWNLQETVDLDQ | 495 |
| WP_002378009 | 428 | QAIIHRQAAYYPFLKE--NQE KIEQLVTFRIPYYVGPL SKGDa-NTFAWLKR---QSEEPIRPWNLQETVDLDQ | 495 |
| WP_002407324 | 428 | QAIIHRQAAYYPFLKE--NQE KIEQLVTFRIPYYVGPL SKGDa-STFAWLKR---QSEEPIRPWNLQETVDLDQ | 495 |
| WP_002413717 | 428 | QAIIHRQAAYYPFLKE--NQE KIEQLVTFRIPYYVGPL SKGDa-STFAWLKR---QSEEPIRPWNLQETVDLDQ | 495 |
| WP_010775580 | 430 | QAIIHRQAAYYPFLKE--NOK KIEQLVTFRIPYYVGPL SKGDa-STFAWLKR---QSEEPIRPWNLQETVDLDO | 497 |
| WP_010818269 | 428 | QAIIHRQAAYYPFLKE--NQE KIEQLVTFRIPYYVGPL SKGDa-STFAWLKR---QSEEPIRPWNLQETVDLDQ | 495 |
| WP_010824395 | 428 | QAIIHRQAAYYPFLKE--NQE KIEQLVTFRIPYYVGPL SKGDa-STFAWLKR---QSEEPIRPWNLQETVDLDQ | 495 |
| WP_016622645 | 428 | QAIIHRQAAYYPFLKE--NQE KIEQLVTFRIPYYVGPL SKGDa-STFAWLKR---QSEEPIRPWNLQETVDLDQ | 495 |
| WP_033624816 | 428 | QAIIHRQAAYYPFLKE--NQK KIEQLVTFRIPYYVGPL SKGDa-STFAWLKR---QSEEPIRPWNLQETVDLDQ | 495 |
| WP_033625576 | 428 | QAIIHRQAAYYPFLKE--NQE KIEQLVTFRIPYYVGPL SKGDa-STFAWLKR---QNEKPIRPWNLQETVDLDO | 495 |
| WP_033789179 | 428 | QAIIHRQAAYYPFLKE--NQK KIEQLVTFRIPYYVGPL SKGDa-STFAWLKR---QSEEPIRPWNLQETVDLDQ | 495 |
| WP_002310644 | 424 | RAIIANQKKHYPFLKE--EQE KLESLLTFKIPYYVGPL AKKQenSPFAWLIR---KSEEKIKPWNLPEIVDMEG | 492 |
| WP_002312694 | 425 | RAIIANQKKHYPFLKE--EQE KLESLLTFKIPYYVGPL AKKQenSPFAWLIR---KSEEKIKPWNLPEIVDMEG | 493 |
| WP_002314015 | 425 | RAIIANQKKHYPFLKE--EQE KLESLLTFKIPYYVGPL AKKQenSPFAWLIR---KSEEKIKPWNLPEIVDMEG | 493 |
| WP_002320716 | 425 | RAIIANQKKHYPFLKE--EQE KLESLLTFKIPYYVGPL AKKQenSPFAWLIR---KSEEKIKPWNLPEIVDMEG | 493 |
| WP_002330729 | 424 | RAIIANQKKHYPFLKE--EQE KLESLLTFKIPYYVGPL AKKQenSPFAWLIR---KSEEKIKPWNLPEIVDMEG | 492 |
| WP_002335161 | 425 | RAIIANQKKHYPFLKE--EQE KLESLLTFKIPYYVGPL AKKQenSPFAWLIR---KSEEKIKPWNLPEIVDMEG | 493 |
| WP_002345439 | 425 | RAIIANQKKHYPFLKE--EQE KLESLLTFKIPYYVGPL AKKQenSPFAWLIR---KSEEKIKPWNLPEIVDMEG | 493 |
| WP_034867970 | 418 | KAIIDQQKQHYPFLEE--AGP KIIALFKFRIPYYVGPL AKEQeaSSFAWIER---KTAEKINPWNFSEVVDIEK | 486 |
| WP_047937432 | 425 | RAIIANQKKHYPFLKE--EQE KLESLLTFKIPYYVGPL AKKQenSPFAWLIR---KSEEKIKPWNLPEIVDMEG | 493 |
| WP_010720994 | 418 | KAIIDQQKQHYPFLEE--AGP KIIALFKFRIPYYVGPL AKEQeaSSFAWIER---KTAEKINPWNFSEVVDIEK | 486 |
| WP_010737004 | 418 | KAIIDQQKQHYPFLEE--AGP KIIALFKFRIPYYVGPL AKEQeaSSFAWIER---KTAEKINPWNFSEVVDIEK | 486 |
| WP_034700478 | 418 | KAIIDQQKQHYPFLEE--AGP KIIALFKFRIPYYVGPL AKEQeaSSFAWIER---KTAEKINPWNFSEVVDIEK | 486 |
| WP_007209003 | 422 | RAILRKQEKYYSFLKE--NHE KIEQIFKVRIPYYVGPL AKHNeqSRFAWNIR---KSDEPIRPWNMNDVVDENA | 490 |
| WP_023519017 | 418 | REIMDRQKRFYPFLKG--AQG KIEKLLTFRIPYYVGPL AQEGq-SPFAWIKR---KSPSQITPWNFAEVVDKEN | 485 |
| WP_010770040 | 423 | EAIIQKQATYYPFLAD--NKE EMKQLVTFRIPYYVGPL ADGN--SPFAWLER---ISSEPIRPGNLAEVVDIKK | 489 |
| WP_048604708 | 419 | KAILHHQAMYYPFLQE--KFS NFVDLLTFRIPYYVGPL ANGN--SRFSWLSR---KSDEPIRPWNLAEVVDLSK | 485 |
| WP_010750235 | 418 | KAIIDQQKQYYPFLEK--SKE KMIQLLTFRIPYYVGPL AQDKetSSFAWLER---KTTEKIKPWNAKDVIDYGA | 486 |
| AII16583 | 459 | HAILRRQEDFYPFLKD--NRE KIEKILTFRIPYYVGPL ARG-n-SRFAWMTR---KSEETITPWNFEEVVDKGA | 525 |
| WP_029073316 | 436 | NKILENQSVYYSDLKD--NED KIRSILTFRIPYYFGPL ITKDr--QFDWIIKkegKENERILPWNANEIVDVDK | 506 |
| WP_031589969 | 436 | NKILENQSVYYSDLKD--NED KIRSILTFRIPYYFGPL ITKDr--QFDWIIKkegKENERILPWNANEIVDVDK | 506 |
| KDA45870 | 418 | VAIVENQGKYYPFLRE--NKD KFEKILNFRIPYYVGPL ARGN--SKFAWLTR--a-GEGKITPYNFDEMIDKET | 484 |
| WP_039099354 | 426 | DRIIENQQQYYPWLAE-1NPN KLDELVAFRVPYYVGPL QQQSsdAKFAWMIR---KAEGQITPWNEDDKVDRQA | 509 |
| AKP02966 | 416 | EKIIDNQSQYYPFLKE--NKE KLLSILSFRIPYYVGPL -QSSekNPFAWMER---KSNGHARPWNFDEIVDREK | 483 |
| WP_010991369 | 426 | EAILHQQAKYYPFLKE--NYD KIKSLVTFRIPYFVGPL ANGQ--SEFAWLTR---KADGEIRPWNIEEKVDFGK | 492 |
| WP_033838504 | 426 | EAILHQQAKYYPFLKE--NYD KIKSLVTFRIPYFVGPL ANGQ--SEFAWLTR---KADGEIRPWNIEEKVDFGK | 492 |
| EHN60060 | 429 | EAILHQQAKYYPFLKE--NYD KIKSLVTFRIPYFVGPL ANGQ--SEFAWLTR---KADGEIRPWNIEEKVDFGK | 495 |
| EFR89594 | 195 | EAILHQQAKYYPFLKE--NYD KIKSLVTFRIPYFVGPL ANGQ--SEFAWLTR---KADGEIRPWNIEEKVDFGK | 261 |
| WP_038409211 | 426 | EAILHQQAKYYPFLRK--DYE KIRSLVTFRIPYFIGPL ANGQ--SDFAWLTR---KADGEIRPWNIEEKVDFGK | 492 |
| EFR95520 | 45 | EAILHQQAKYYPFLRK--DYE KIRSLVTFRIPYFIGPL ANGQ--SDFAWLTR---KADGEIRPWNIEEKVDFGK | 111 |
| WP_003723650 | 426 | EAIIHQQAKYYPFLKE--DYD KIKSLVTFRIPYFVGPL ANGQ--SEFAWLTR---KADGEIRPWNIEEKVDFGK | 492 |
| WP_003727705 | 426 | EAILHQQAKYYPFLRE--GYD KIKSLVTFRIPYFVGPL ANGQ--SEFAWLTR---KDDGEIRPWNIEEKVDFGK | 492 |
| WP_003730785 | 426 | EAILHQQAKYYPFLRE--GYD KIKSLVTFRIPYFVGPL ANGQ--SEFAWLTR---KDDGEIRPWNIEEKVDFGK | 492 |
| WP_003733029 | 426 | EAILHQQAKYYPFLRE--DYE KIKSLVTFRIPYFVGPL AKGQ--SEFAWLTR---KADGEIRPWNIEEKVDFGK | 492 |
| WP_003739838 | 426 | EAILHQQAKYYPFLKE--AYD KIKSLVTFRIPYFVGPL ANGQ--SDFAWLTR---KADGEIRPWNIEEKVDFGK | 492 |
| WP_014601172 | 426 | EAIIHQQAKYYPFLRE--DYE KIKSLVTFRIPYFVGPL AKGQ--SEFAWLTR---KADGEIRPWNIEEKVDFGK | 492 |
| WP_023548323 | 426 | EAILHQQAKYYPFLRE--DYE KIKSLVTFRIPYFVGPL AKGQ--SEFAWLTR---KADGEIRPWNIEEKVDFGK | 492 |
| WP_031665337 | 426 | EAIIHQQAKYYTFLKE--DYD KIKSLVTFRIPYFVGPL ANGQ--SEFAWLTR---KADGEIRPWNIEEKVDFGK | 492 |
| WP_031669209 | 426 | EAILHQQAKYYPFLRE--DYE KIKSLVTFRIPYFVGPL AKGQ--SEFAWLTR---KADGEIRPWNIEEKVDFGK | 492 |
| WP_033920898 | 426 | EAIIHQQAKYYPFLRE--DYE KIKSLVTFRIPYFVGPL AKGQ--SEFAWLTR---KADGEIRPWNIEEKVDEGK | 492 |
| AKI42028 | 429 | EAIIHQQAKYYPFLRE--DYE KIKSLVTFRIPYFVGPL AKGQ--SEFAWLTR---KADGEIRPWNIEEKVDFGK | 495 |
| AKI50529 | 429 | EAIIHQQAKYYPFLRE--DYE KIKSLVTFRIPYFVGPL AKGQ--SEFAWLTR---KADGEIRPWNIEEKVDFGK | 495 |
| EFR83390 | | --------------------- ----------------- ------------------------------------ | |
| WP_046323366 | 426 | EAILHQQAKYYPFLKV--DYE KIKSLVTFRIPYFVGPL ANGQ--SEFSWLTR---KADGEIRPWNIEEKVDFGK | 492 |
| AKE81011 | 436 | HAILRRQEDFYPFLKD--NRE KIEKILTFRIPYYVGPL ARG-n-SRFAWMTR---KSEETITPWNFEEVVDKGA | 502 |
| CUO82355 | 434 | IKIIDNQAKYYPVLKE--KRE QLLSILTFRIPYYFGPL ETSEh----AWIKRlegKENQRILPWNYQDTVDVDA | 502 |
| WP_033162887 | 435 | IQIIDNQSVYYPQLKE--NRD KLISILEFRIPYYFGPL AHSE----FAWIKKfedKQKERILPWNYDQIVDIDA | 503 |
| AGZ01981 | 453 | HAILRRQEDFYPFLKD--NRE KIEKILTFRIPYYVGPL ARG-n-SRFAWMTR---KSEETITPWNFEEVVDKGA | 519 |
| AKA60242 | 420 | HAILRRQEDFYPFLKD--NRE KIEKILTFRIPYYVGPL ARG-n-SRFAWMTR---KSEETITPWNFEEVVDKGA | 486 |
| AKS40380 | 420 | HAILRRQEDFYPFLKD--NRE KIEKILTFRIPYYVGPL ARG-n-SRFAWMTR---KSEETITPWNFEEVVDKGA | 486 |
| 4UN5_B | 424 | HAILRRQEDFYPFLKD--NRE KIEKILTFRIPYYVGPL ARG-n-SRFAWMTR---KSEETITPWNFEEVVDKGA | 490 |
| WP_010922251 | 487 | 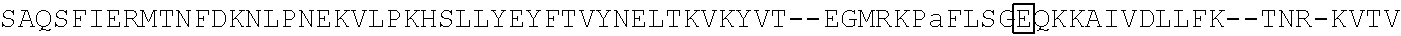 | 561 |
| WP_039695303 | 490 | SAEKFITRMTLNDLYLPEEKVLPKHSHVYETYAVYNELTKIKYVN--EQGKES-FFDSNMKQEIFDHVFK--ENR-KVTK | 563 |
| WP_045635197 | 487 | SAEDFINKMTNYDLYLPEEKVLPKHSLLYETFAVYNELTKVKFIA--EGLRDYqFLDSGQKKQIVNOLFK--ENR-KVTE | 561 |
| 5AXW_A | 230 | --KEWYEMLMGHCTYFPEELRSVKYAYNADLYNALNDLNNLVITR--DENEKLeYYE---KFQIIENVFK--QKK-KPTL | 299 |
| WP_009880683 | 171 | SAQSFIERMTNFDKNLPNEKVLPKHSLLYEYFTVYNELTKVKYVT--EGMRKPaFLSGEQKKAIVDLLFK--TNR-KVTV | 245 |
| WP_010922251 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_011054416 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_011284745 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_011285506 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_011527619 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_012560673 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_014407541 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_020905136 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_023080005 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_023610282 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_030125963 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_030126706 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_031488318 | 487 | SAQSFIERMTNFDKNLPNEKVLPKHSLLYEYFTVYNELTKVKYVT--EGMRKPeFLSGEQKKAIVDLLFK--TNR-KVTV | 561 |
| WP_032460140 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_032461047 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_032462016 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_032462936 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_032464890 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_033888930 | 312 | SAQSFIERMTNFDKNLPNEKVLPKHSLLYEYFTVYNELTKVKYVT--EGMRKPaFLSGEQKKAIVDLLFK--TNR-KVTV | 386 |
| WP_038431314 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_038432938 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_038434062 | 487 | SAQSFIERMTNFDKNLPNEKVLPKHSLLYEYFTVYNELTKVKYVT--EGMRKPaFLSGEQKKAIVDLLFK--TNR-KVTV | 561 |
| BAQ51233 | 398 | SAQSFIERMTNFDKNLPNEKVLPKHSLLYEYFTVYNELTKVKYVT--EGMRKPaFLSGEQKKAIVDLLFK--TNR-KVTV | 472 |
| KGE60162 | | -------------------------------------------------------------------------------- | |
| KGE60856 | | -------------------------------------------------------------------------------- | |
| WP_002989955 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_003030002 | 487 | SAEKFITRMTLNDLYLPEEKVLPKHSLLYETFTVYNELTKVKYVN--EQGEAK-FFDANMKQEIFDHVFK--ENR-KVTK | 560 |
| WP_003065552 | 490 | SAEKFITRMTLNDLYLPEEKVLPKHSHVYETYAVYNELTKIKYVN--EQGKDS-FFDSNMKQEIFDHVFK--ENR-KVTK | 563 |
| WP_001040076 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_001040078 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040080 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040081 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040083 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040085 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040087 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040088 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040089 | 488 | SAEAFHIRMTNNDFYLPEEKVLPKHSLIYEKFTVYNELTKVRYKN--EQGETY-FFDSNIKQEIFDGVFK--EHR-KVSK | 561 |
| WP_001040090 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040091 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040092 | 488 | SAEAFIHRMTNNDLYLPEEKVLPKHSLIYEKFTVYNELTKVRYKN--EQGETY-FFDSNVKQEIFDGVFK--EHR-KVSK | 561 |
| WP_001040094 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_001040095 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_001040096 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_001040097 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_001040098 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_001040099 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_001040100 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_001040104 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040105 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_001040106 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_001040107 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_001040108 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_001040109 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_001040110 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_015058523 | 488 | SAEAFIHRMTNNDLYLPEEKVLPKHSLIYEKFTVYNELTKVRYKN--EQGETY-FFDSNVKQEIFDGVFK--EHR-KVSK | 561 |
| WP_017643650 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_017647151 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_017648376 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_017649527 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_017771611 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_017771984 | 488 | SAEAFIHRMTNNDFYLPEEKVLPKHSLIYEKFTVYNELTKVRYKN--EQGETY-FFDSNIKQEIFDGVFK--EHR-KVSK | 561 |
| CFQ25032 | 488 | SAEAFIHRMTNNDFYLPEEKVLPKHSLIYEKFTVYNELTKVRYKN--EQGETY-FFDSNIKQEIFDGVFK--EHR-KVSK | 561 |
| CFV16040 | 488 | SAEAFIHRMTNNDFYLPEEKVLPKHSLIYEKFTVYNELTKVRYKN--EQGETY-FFDSNIKQEIFDGVFK--EHR-KVSK | 561 |
| KLJ37842 | 488 | SAEAFIHRMTNNDFYLPEEKVLPKHSLIYEKFTVYNELTKVRYKN--EQGETY-FFDSNIKQEIFDGVFK--EHR-KVSK | 561 |
| KLJ72361 | 488 | SAEAFIHRMTNNDFYLPEEKVLPKHSLIYEKFTVYNELTKVRYKN--EQGETY-FFDSNIKQEIFDGVFK--EHR-KVSK | 561 |
| KLL20707 | 488 | SAEAFIHRMTNNDFYLPEEKVLPKHSLIYEKFTVYNELTKVRYKN--EQGETY-FFDSNIKQEIFDGVFK--EHR-KVSK | 561 |
| KLL42645 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_047207273 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_047209694 | 488 | SAEAFIHRMTNNDLYLPEEKVLPKHSLIYEKFTVYNELTKVRFLA--EGFKDFqFLNRKQKETIFNSLFK--EKR-KVTE | 562 |
| WP_050198062 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_050201642 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_050204027 | 488 | SAEAFIHRMTNNDLYLPEEKVLPKHSLIYEKFTVYNELTKVRYKN--EQGETY-FFDSNIKQEIFDGVFK--EHR-KVSK | 561 |
| WP_050881965 | 488 | SAEAFIHRMTNNDFYLPEEKVLPKHSLIYEKFTVYNELTKVRYKN--EQGETY-FFDSNIKQEIFDGVFK--EHR-KVSK | 561 |
| WP_050886065 | 488 | SAEAFIHRMTNNDFYLPEEKVLPKHSLIYEKFTVYNELTKVRYKN--EQGETY-FFDSNIKQEIFDGVFK--EHR-KVSK | 561 |
| AHN30376 | 488 | SAEAFIHRMTNNDLYLPEEKVLPKHSLIYEKFTVYNELTKVRYKN--EQGETY-FFDSNVKQEIFDGVFK--EHR-KVSK | 561 |
| EAO78426 | 488 | SAEAFIHRMTNNDFYLPEEKVLPKHSLIYEKFTVYNELTKVRYKN--EQGETY-FFDSNIKQEIFDGVFK--EHR-KVSK | 561 |
| CCW42055 | 488 | SAEAFIHCMTNNDLYLPEEKVLPKHSLIYEKFTVYNELTKVRYKN--EQGETY-FFDSNIKQEIFDGVFK--EYR-KVSK | 561 |
| WP_003041502 | 487 | SAEKFITRMTLNDLYLPEEKVLPKHSLLYETFTVYNELTKVKYVN--EQGEAK-FFDANMKQEIFDHVFK--ENR-KVTK | 560 |
| WP_037593752 | 488 | SAEKFITRMTLNDLYLPEEKVLPKHSPLYETFTVYNELTKVKYVN--EQGEAK-FFDTNMKQEIFDHVFK--ENR-KVTK | 561 |
| WP_049516684 | 488 | SAEKFITRMTLNDLYLPEEKVLPKHSPLYETFTVYNELTKVKYVN--EQGEAK-FFDTNMKQEIFDHVFK--ENR-KVTK | 561 |
| GAD46167 | 487 | SAEKFITRMTLNDLYLPEEKVLPKHSPLYETFTVYNELTKVKYVN--EQGEAK-FFDTNMKQEIFDHVFK--ENR-KVTK | 560 |
| WP_018363470 | 488 | SAEKFITRMTLNDLYLPEEKVLPKHSHVYETFAVYNELTKVKYVN--EQGKDS-FFDSNMKQEIFDHVFK--ENR-KVTK | 561 |
| WP_003043819 | 497 | SAQSFIERMTNFDEQLPNKKVLPKHSLLYEYFTVYNELTKVKYVT--ERMRKPeFLSGEQKKAIVDLLFK--TNR-KVTV | 571 |
| WP_006269658 | 487 | SAEKFITRMTLNDLYLPEEKVLPKHSPLYEAFTVYNELTKVKYVN--EQGEAK-FFDTNMKQEIFDHVFK--ENR-KVTK | 560 |
| WP_048800889 | 487 | SAEKFITRMTLNDLYLPEEKVLPKHSLLYEIFTVYNELTKVKYVN--EQGEAK-FFDANMKQEIFDHVFK--ENP-KVTK | 560 |
| WP_012767106 | 487 | SAQSFIERMTNFDKNLPNEKVLPKHSLLYEYFTVYNELTKVKYVT--EGMRKPeFLSGKQKEAIVDLLFK--TNR-KVTV | 561 |
| WP_014612333 | 487 | SAQSFIERMTNFDKNLPNEKVLPKHSLLYEYFTVYNELTKVKYVT--EGMRKPeFLSGKQKEAIVDLLFK--TNR-KVTV | 561 |
| WP_015017095 | 487 | SAQSFIERMTNFDKNLPNEKVLPKHSLLYEYFTVYNELTKVKYVT--EGMRKPeFLSGKQKEAIVDLLFK--TNR-KVTV | 561 |
| WP_015057649 | 487 | SAQSFIERMTNFDKNLPNEKVLPKHSLLYEYFTVYNELTKVKYVT--EGMRKPeFLSGKQKEAIVDLLFK--TNR-KVTV | 561 |
| WP_048327215 | 487 | SAQSFIERMTNFDKNLPNEKVLPKHSLLYEYFTVYNELTKVKYVT--EGMRKPeFLSGKQKEAIVDLLFK--TNR-KVTV | 561 |
| WP_049519324 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_012515931 | 487 | SAQIFIEKMTKNDLYLPNEKVLPKHSLLYETFTVYNELTKVKYAT--EGMTRPqFLSADQKQAIVDLLFK--TNR-KVTV | 561 |
| WP_021320964 | 487 | SAQIFIEKMTKNDLYLPNEKVLPKHSLLYETFTVYNELTKVKYAT--EGMTRPqFLSADQKQAIVDLLFK--TNR-KVTV | 561 |
| WP_037581760 | 487 | SAQIFIEKMTKNDLYLPNEKVLPKHSLLYETFTVYNELTKVKYAT--EGMTRPqFLSADQKQAIVDLLFK--TNR-KVTV | 561 |
| WP_004232481 | 487 | SAEKFITRMTLNDLYLPEEKVLPKHSYVYETFAVYNELTKIKYVN--EQGKSF-FFDANMKQEIFDHVFK--ENR-KVTK | 560 |
| WP_009854540 | 488 | SAEKFITRMTLNDLYLPEEKVLPKHSHVYETYAVYNELTKIKYVN--EQGKES-FFDSNMKQEIFDHVFK--ENR-KVTK | 561 |
| WP_012962174 | 488 | SAEKFITRMTLNDLYLPEEKVLPKHSLVYETYTVYNELTKVKYVN--EQGKSN-FFDANMKQEIFEHVFK--ENR-KVTK | 561 |
| WP_039695303 | 490 | SAEKFITRMTLNDLYLPEEKVLPKHSHVYETYAVYNELTKIKYVN--EQGKES-FFDSNMKQEIFDHVFK--NR-KVTK | 563 |
| WP_014334983 | 487 | SAEKFITRMTLNDLYLPEEKVLPKHSHVYETFTVYNELTKIKYVN--EQGESF-FFDANMKQEIFDHVFK--ENR-KVTK | 560 |
| WP_003099269 | 487 | SARAFIERMTNFDTYLPEEKVLPKHSPLYEMFMVYNELTKVKYQT--EGMKRPVFLSSEDKEEIVNLLFK--KER-KVTV | 561 |
| AHY15608 | 487 | SARAFIERMTNFDTYLPEEKVLPKHSPLYEMFMVYNELTKVKYQT--EGMKRPVFLSSEDKEEIVNLLFK--KER-KVTV | 561 |
| AHY17476 | 487 | SARAFIERMTNFDTYLPEEKVLPKHSPLYEMFMVYNELTKVKYQT--EGMKRPVFLSSEDKEEIVNLLFK--KER-KVTV | 561 |
| ESR09100 | | -------------------------------------------------------------------------------- | |
| AGM98575 | 487 | SARAFIERMTNFDTYLPEEKVLPKHSPLYEMFMVYNELTKVKYQT--EGMKRPVFLSSEDKEEIVNLLFK--KER-KVTV | 561 |
| ALF27331 | 487 | SAEAFINRMTNYDLYLPNQKVLPRHSLLYEKFTVYNELTKVKYKT--EQGKTA-FFDANMKQEIFDGVFK--VYR-KVTK | 560 |
| WP_018372492 | 500 | SASRFIERMTLHDLYLPDEKVLPRHSLIYEKYTVFNELTKVRFTP--EGGKEV-YFSKIDKENIFDSLFK--RYR-KVTK | 573 |
| WP_045618028 | 488 | SAEDFINKMTNYDLYLPEEKVLPKHSLLYETFAVYNELTKVKFIA--EGLRDYqFLDSGQKQQIVTQLFK--EKR-KVTE | 562 |
| WP_045635197 | 487 | SAEDFINKMTNYDLYLPEEKVLPKHSLLYETFAVYNELTKVKFIA--EGLRDYqFLDSGQKKQIVNQLFK--ENR-KVTE | 561 |
| WP_002263549 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002263887 | 487 | SAEAFINRMTNYDLYLPNQKVLPKHSLLYEKFTVYNELIKVKYKT--EQGKTA-FFDANMKQEIFDGVFK--VYR-KVTK | 560 |
| WP_002264920 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_002269043 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_002269448 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002271977 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002272766 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002273241 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002275430 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002276448 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002277050 | 487 | SAQAFIEHMTNNDLYLPNEKVLPKHSPLYEKYTVYNELTKIKYVT--EIGEAK-FFDANLKQEIFDGLFK--HER-KVTK | 560 |
| WP_002277364 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002279025 | 487 | SAEAFINRMTNYDLYLPNQKVLPKHSLLYEKFTVYNELTKVKYKT--EQGETA-FFDANMKQEIFDGVFK--VYR-KVTK | 560 |
| WP_002279859 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_002280230 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002281696 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002282247 | 487 | SAQAFIEHMTNNDLYLPNEKVLPKHSPLYEKYTVYNELTKIKYVT--EIGEAK-FFDANLKQEIFDGLFK--HER-KVTK | 560 |
| WP_002282906 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002283846 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_002287255 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002288990 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_002289641 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002290427 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002295753 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002296423 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_002304487 | 497 | SAEKFITRMTLNDLYLPEEKVLPKHSLLYETFTVYNELTKVKYVN--EQGEAK-FFDANMKQEIFDHVFK--ENR-KVTK | 570 |
| WP_002305844 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002307203 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_002310390 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_002352408 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_012997688 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_014677909 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_019312892 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_019313659 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_019314093 | 487 | SVEAFINRMTNYDLYLPNQKVLPKHSLLYEKFTVYNELTKVKYKT--EQGKTA-FFDANMKQEIFDGVFK--VYR-KVTK | 560 |
| WP_019315370 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_019803776 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_019805234 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_024783594 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_024784288 | 487 | SAQAFIEHMTNNDLYLPNEKVLPKHSPLYEKYTVYNELTKIKYVT--EIGEAK-FFDANLKQEIFDGLFK--HER-KVTK | 560 |
| WP_024784666 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_024784894 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_024786433 | 487 | SAQAFIEHMTNNDLYLPNEKVLPKHSPLYEKYTVYNELTKIKYVT--EIGEAK-FFDANLKQEIFDGLFK--HER-KVTK | 560 |
| WP_049473442 | 487 | SAEAFINRMTNYDLYLPNQKVLPKHSLLYEKFTVYNELTKVKYKT--EQGKTA-FFDANMKQEIFDGVFK--VYR-KVTK | 560 |
| WP_049474547 | 487 | SAEAFINRMTNYDLYLPNQKVLPKHSLLYEKFTVYNELTKVKYKT--EQGKTA-FFDANMKQEIFDGVFK--VYR-KVTK | 560 |
| EMC03581 | 480 | SAEAFINRMTNYDLYLPNQKVLPKHSLLYEKFTVYNELTKVKYKT--EQGKTA-FFDANMKQEIFDGVFK--VYR-KVTK | 553 |
| WP_000428612 | 488 | SAESFINKMTNYDLYLPEEKVLPKHSLLYETFAVYNELTKVKFIA--EGLRDYqFLDSROKKDIFYTLFKaeDKR-KVTE | 564 |
| WP_000428613 | 488 | SAEDFINKMTNYDLYLPEEKVLPKHSLLYETFAVYNELTKVKFIA--EGLRDYqFLDSGQKKQIVTQLFK--EKR-KVTE | 562 |
| WP_049523028 | 487 | SAEDFIHRMTNYDLYLPEEKVLPKHSLLYETFTVYNELTKVKYIA--EGMKDYqFLDSGOKKQIVNOLFK--EKR-KVTE | 561 |
| WP_003107102 | 456 | SAKVFIERMTNFDTYLPEEKVLPKHSLLYEMFTVYNELTKVKYQA--EGMRKPeFLSSEEKIEIVSNLFK--TER-KVTV | 530 |
| WP_054279288 | 489 | SAQAFIERMTNFDEYLPQEKVLPKHSLTYEYFTVYNELTKVKYVT--EGMTKPeFLSAGQKEQIVELLFK--KYR-KVTV | 563 |
| WP_049531101 | 488 | SAEAFINKMTNYDLYLPEEKVLPKHSLLYETFAVYNELTKVKFIA--EGLRDYqFLDSGQKKKIINQLFK--EKR-KVTE | 562 |
| WP_049538452 | 488 | SAEDFINKMTNYDLYLPEEKVLPKHSLLYETFAVYNELTKVKFIA--EGLRDYqFLDSGQKKQIVNOLFK--EKR-KVTE | 562 |
| WP_049549711 | 488 | SAEDFINKMTNYDLYLPEEKVLPKHSLLYETFAVYNELTKVKFIA--EGLRDYqFLDSGQKKQIVNQLFK--EKR-KVTE | 562 |
| WP_007896501 | 490 | SAQAFIEGMTNYDTYLPEEKVLPKHSPLYEMFTVYNELTKVKYIA--ENMTKPIYLSAEQKEAIIDHLFK--QTR-KVTV | 564 |
| EFR44625 | 442 | SAQAFIEGMTNYDTYLPEEKVLPKHSPLYEMFTVYNELTKVKYIA--ENMTKP1YLSAEQKEAIIDHLFK--QTR-KVTV | 516 |
| WP_002897477 | 487 | SAEDFIHRMTNYDLYLPEEKVLPKHSLLYETFAVYNELTKVKFIA--EGLRDYqFLDSGQKKQIVNQLFK--EKR-KVTE | 561 |
| WP_002906454 | 487 | SAEDFINKMTNYDLYLPEEKVLPKHSLLYETFAVYNELTKVKFIA--EGLRDYqFLDSGQKKQIVNQLFK--DKR-KVTE | 561 |
| WP_009729476 | 488 | SAEDFINKMTNYDLYLPEEKVLPKHSLLYETFAVYNELTKVKFIA--EGLRDYqFLDSGQKKQIVTQLFK--EKR-KVTE | 562 |
| COR24647 | 488 | SAQAFIERMINNDLYLPDQKVLPKHSLLYQKFAVYNELTKIKYVT--ETGEAR-LFDVELKKEIFDGLFK--KER-KVTK | 561 |
| WP_000066813 | 488 | SAEDFINKMTNYDLYLPEEKVLPKHSLLYETFAVYNELTKVKFIA--EGLTRYqFLDKKQKKDIFDTFFKaeNKR-KVTE | 564 |
| WP_009754323 | 488 | SAESFINKMTNYDLYLPEEKVLPKHSLLYETFAVYNELTKVKFIA--EGLRDYqFFDSGQKKQIVNOLFK--EKR-KVTE | 562 |
| WP_044674937 | 487 | SAENFITRMTNYDQYLPDQKVLPKHSLLYEKFAVYNELTKVKFIA--EGMRDYqFLDSGQKKDIVKTLFK--TKR-KVTA | 561 |
| WP_044676715 | 487 | SAENFITRMTNYDQYLPDQKVLPKHSLLYEKFAVYNELTKVRYVT--EQGKSF-FFDANMKQEIFDGVFK--VYR-KVTK | 560 |
| WP_044680361 | 487 | SAENFITRMTNYDQYLPDQKVLPKHSLLYEKFAVYNELTKVRYVT--EQGKSF-FFDANMKQEIFDGVFK--VYR-KVTK | 560 |
| WP_044681799 | 487 | SAENFITRMTNYDQYLPDQKVLPKHSLLYEKFAVYNELTKVKFIA--EGMRDYqFLDSGQKKDIVKTLFK--TKR-KVTA | 561 |
| WP_049533112 | 487 | SAEKFITRMTLNDLYLPEEKVLPKHSLLYETFTVYNELTKVKYVN--EQGEAK-FFDANMKQEIFDHVFK--ENR-KVTK | 560 |
| WP_029090905 | 472 | SSEAFIKRMINKCTYLIHEDVIPKHSFSYAKFEVLNELNKIRLDG------KP--IDIPLKKRIFEGLFL---EKtKVTQ | 540 |
| WP_006506696 | 499 | TAEGFIKRMRSYCTYFPDEEVLPKNSLIVSKYEVYNELNKIRVDD--------kLLEVDVKNDIYNELFM--KNK-TVTE | 567 |
| AIT42264 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_034440723 | 494 | SAELFIENLTSRDTYLPDEPVLPKRSLIYQKFTIFNELTKISYID--ERGILQ-NFSSREKIAIFNDLFK---NKsKVTK | 567 |
| AKQ21048 | 487 | SAQSFIERMTNFDKNLPNEKVLPKHSLLYEYFTVYNELTKVKYVT--EGMRKPaFLSGEQKKAIVDLLFK--TNR-KVTV | 561 |
| WP_004636532 | 489 | SAEKFIERMTNMDTYLLEEKVLPKRSLLYQTFEVYNELTKVRYTN--EQGKTE-KLNRQQKAEIIETLFK-qKNR--VRE | 562 |
| WP_002364836 | 496 | SATAFIERMTNFDTYLPSEKVLPKHSLLYEKFMVENELTKISYTD--DRGIKA-NFSGKEKEKIFDYLFK--TRR-KVKK | 569 |
| WP_016631044 | 447 | SATAFIERMTNFDTYLPSEKVLPKHSLLYEKFMVFNELTKISYTD--DRGIKA-NFSGKEKEKIFDYLFK--TRR-KVKK | 520 |
| EMS75795 | 232 | SAMKFIQRMINYDTYLPTEKVLPKHSILYQKYTIFNELTKVAYKD--ERGIKH-QFSSKEKREIFKELFQ--KQR-KVTV | 305 |
| WP_002373311 | 496 | SATAFIERMTNFDTYLPSEKVLPKHSLLYEKFMVFNELTKISYTD--DRGIKA-NFSGKEKEKIFDYLFK--TRR-KVKK | 569 |
| WP_002378009 | 496 | SATAFIERMTNFDTYLPSEKVLPKHSLLYEKFMVFNELTKISYTD--DRGIKA-NFSGKEKEKIFDYLFK--TRR-KVKK | 569 |
| WP_002407324 | 496 | SATAFIERMTNFDTYLPSEKVLPKHSLLYEKFMVFNELTKISYTD--DRGIKA-NFSGKEKEKIEDYLFK--TRR-KVKK | 569 |
| WP_002413717 | 496 | SATAFIERMTNFDTYLPSEKVLPKHSLLYEKFMVFNELTKISYTD--DRGIKA-NFSGKEKEKIFDYLFK--TRR-KVKK | 569 |
| WP_010775580 | 498 | SATAFIERMTNFDTYLPSEKVLPKHSLLYEKFMVFNELTKISYTD--DRGIKA-NFSGKEKEKIFDYLFK--TRR-KVKK | 571 |
| WP_010818269 | 496 | SATAFIERMTNFDTYLPSEKVLPKHSLLYEKFMVFNELTKISYTD--DRGIKA-NFSGKEKEKIFDYLFK--TRR-KVKK | 569 |
| WP_010824395 | 496 | SATAFIERMTNFDTYLPSEKVLPKHSLLYEKFMVENELTKISYTD--DRGIKA-NFSGKEKEKIEDYLFK--TRR-KVKK | 569 |
| WP_016622645 | 496 | SATAFIERMTNFDTYLPSEKVLPKHSLLYEKFMVFNELTKISYTD--DRGIKA-NFSGKEKEKIEDYLFK--TRR-KVKK | 569 |
| WP_033624816 | 496 | SATAFIERMTNFDTYLPSEKVLPKHSLLYEKFMVFNELTKISYTD--DRGIKA-NFSGKEKEKIFDYLFK--TRR-KVKK | 569 |
| WP_033625576 | 496 | SATAFIERMTNFDTYLPSEKVLPKHSLLYEKFMVFNELTKISYTD--DRGIKA-NFSGKEKEKIFDYLFK--TRR-KVKK | 569 |
| WP_033789179 | 496 | SATAFIERMTNFDTYLPSEKVLPKHSLLYEKFMVFNELTKISYTD--DRGIKA-NFSGKEKEKIFDYLFK--TRR-KVKK | 569 |
| WP_002310644 | 493 | SAVRFIERMINTDMYMPHNKVLPKNSLLYQKFSIYNELTKVRYQD--ERGQMN-YFSSIEKKEIFHELFE--KNR-KVTK | 566 |
| WP_002312694 | 494 | SAVRFIERMINTDMYMPHNKVLPKNSLLYQKFSIYNELTKVRYQD--ERGOMN-YFSSIEKKEIFHELFE--KNR-KVTK | 567 |
| WP_002314015 | 494 | SAVRFIERMNNTDMYIPHNKVLPKNSLLYQKFSIYNELTKVRYQD--ERGOMN-YFSSIEKKEIFHELFE--KNR-KVTK | 567 |
| WP_002320716 | 494 | SAVRFIERMINTDMYIPHNKVLPKNSLLYQKFSIYNELTKVRYQD--ERGOMN-YFSSIEKKEIFHELFE--KNR-KVTK | 567 |
| WP_002330729 | 493 | SAVRFIERMINTDMYIPHNKVLPKNSLLYQKFSIYNELTKVRYQD--ERGOMN-YFSSIEKKEIFHELFE--KNR-KVTK | 566 |
| WP_002335161 | 494 | SAVRFIERMINTDMYMPHNKVLPKNSLLYQKFSIYNELTKVRYQD--ERGOMN-YFSSIEKKEIFHELFE--KNR-KVTK | 567 |
| WP_002345439 | 494 | SAVRFIERMINTDMYIPHNKVLPKNSLLYQKFSIYNELTKVRYQD--ERGOMN-YFSSIEKKEIFHELFE--KNR-KVTK | 567 |
| WP_034867970 | 487 | SAMRFIQRMTKQDTYLPTEKVLPKNSLLYQKYMIFNELTKVSYKD--ERGVKQ-YFSGDEKQQIFKOLFQ--KERgKITV | 561 |
| WP_047937432 | 494 | SAVRFIERMINTDMYMPHNKVLPKNSLLYQKFSIYNELTKVRYQD--ERGOMN-YFSSIEKKEIFHELFE--KNR-KVTK | 567 |
| WP_010720994 | 487 | SAMRFIQRMTKQDTYLPTEKVLPKNSLFYQKYMIFNELTKVSYKD--ERGVKQ-YFSGDEKQQIFKOLFQ--KERgKITV | 561 |
| WP_010737004 | 487 | SAMRFIQRMTKQDTYLPTEKVLPKNSLLYQKYMIFNELTKVSYKD--ERGVKQ-YFSGDEKQQIFKOLFQ--KERgKITV | 561 |
| WP_034700478 | 487 | SAMRFIQRMTKQDTYLPTEKVLPKNSLLYQKYMIFNELTKVSYKD--ERGVKQ-YFSGDEKQQIFKOLFQ--KERgKITV | 561 |
| WP_007209003 | 491 | SAVAFIERMTIKDIYL-NENVLPRHSLIYEKFTVFNELTKVLYAD--DRGVFQ-RFSAEEKEDIFEKLFK--SER-KVTK | 563 |
| WP_023519017 | 486 | SAIEFIERMTNQDTYLPKEKVLPKQSLIYORFMIFNELTKVSYTD--ERGKSH-YFSSEQKRKIFNELFK--QHP-RVTE | 559 |
| WP_010770040 | 490 | SATKFIERMTNFDTYLPTEKVLPKHSMIYEKYMVYNELTKVSYVD--ERGMNQ-RFSGEEKKQIVEELFK--QSR-KVTK | 563 |
| WP_048604708 | 486 | SAELFIERMTNFDLYLPSEKVLPKHSMLYEKYTVYNELTKVTYKD--EQGKVQ-NFSSEEKERIFIDLFK--QHR-KVTK | 559 |
| WP_010750235 | 487 | SATKFIQRMINYDTYLPTEKVLPKYSMLYQKYTIFNELTKVAYKD--DRGIKH-QFSSEEKLRIFQELFK--KQR-RVTK | 560 |
| AII16583 | 526 | SAQSFIERMTNFDKNLPNEKVLPKHSLLYEYFTVYNELTKVKYVT--EGMRKPaFLSGEQKKAIVDLLFK--TNR-KVTV | 600 |
| WP_029073316 | 507 | TADEFIKRMRNFCTYFPDEPVLAKNSLTVSKYEVLNEINKLRIND--------hLIKRDIKDKMLHTLFM--DHK-SISA | 575 |
| WP_031589969 | 507 | TADEFIKRMRNFCTYFPDEPVMAKNSLTVSKYEVLNEINKLRIND--------hLIKRDMKDKMLHTLFM--DHK-SISA | 575 |
| KDA45870 | 485 | SAEDFIKRMTINDLYLPTEPVLPKHSLLYERYTIFNELAGVRYVT--ENGEAK-YFDAQTKRSIFE-LFK1--DR-KVSE | 557 |
| WP_039099354 | 510 | SANEFIKRMTTTDTYLLAEDVLPKQSLIYQRFEVLNELNGLKIDD--QPITTE------LKQAIFTDLFM---QKtSVTV | 578 |
| AKP02966 | 484 | SSNKFIRRMTVTDSYLVGEPVLPKNSLIYQRYEVLNELNNIRITEn1KTNPTGSRLTVETKQHIYNELFK--NYK-KITV | 560 |
| WP_010991369 | 493 | SAVDFIEKMTNKDTYLPKENVLPKHSLCYQKYLVYNELTKVRYIN--DOGKTS-YFSGQEKEQIFNDLFK--QKR-KVKK | 566 |
| WP_033838504 | 493 | SAVDFIEKMTNKDTYLPKENVLPKHSLCYQKYLVYNELTKVRYIN--DQGKTS-YFSGQEKEQIFNDLFK--QKR-KVKK | 566 |
| EHN60060 | 496 | SAVDFIEKMTNKDTYLPKENVLPKHSLCYQKYLVYNELTKVRYIN--DOGKTS-YFSGQEKEQIFNDLFK--QKR-KVKK | 569 |
| EFR89594 | 262 | SAVDFIEKMTNKDTYLPKENVLPKHSLCYQKYLVYNELTKVRYIN--DOGKTS-YFSGQEKEQIFNDLFK--QKR-KVKK | 335 |
| WP_038409211 | 493 | SAIDFIEKMTNKDTYLPKENVLPKHSLCYQKYMVYNELTKIRYID--DQGKTH-HFSGQEKQQIFNGLFK--QQR-KVKK | 566 |
| EFR95520 | 112 | SAIDFIEKMTNKDTYLPKENVLPKHSLCYQKYMVYNELTKIRYID--DOGKTH-HFSGQEKQQIFNGLFK--QQR-KVKK | 185 |
| WP_003723650 | 493 | SAVDFIEKMTNKDTYLPKENVLPKHSLCYQKYMVYNELTKIRYID--DQGKTN-YFSGREKQQVENDLFK--QKR-KVKK | 566 |
| WP_003727705 | 493 | SAVDFIEKMTNKDTYLPKENVLPKHSLCYQKYMVYNELTKIRYID--DOGKIN-YFSGREKQQIFNDLFK--QKR-KVKK | 566 |
| WP_003730785 | 493 | SAVDFIEKMTNKDTYLPKENVLPKHSLCYQKYMVYNELTKIRYID--DOGKTN-YFSGREKQQIFNDLFK--QKR-KVKK | 566 |
| WP_003733029 | 493 | SAVDFIEKMTNKDTYLPKENVLPKHSLCYQKYMVYNELTKVRYID--DOGKTN-YFSGQEKQQIFNDLFK--QKR-KVKK | 566 |
| WP_003739838 | 493 | SAVDFIEKMTNKDTYLPKENVLPKHSLYYQKYMVYNELTKVRYID--DOGKTN-YFSGQEKQQIFNDYFK--QKR-KVSK | 566 |
| WP_014601172 | 493 | SAVDFIEKMTNKDTYLPKENVLPKHSLCYQKYMVYNELTKVRYID--DOGKTN-YFSGQEKQQIFNDLFK--QKR-KVKK | 566 |
| WP_023548323 | 493 | SAVDFIEKMTNKDTYLPKENVLPKHSLCYQKYMVYNELTKIRYID--DQGKTN-YFSGQEKQQIFNDLFK--QKR-KVKK | 566 |
| WP_031665337 | 493 | SAVDFIEKMTNKDTYLPKENVLPKHSLCYQKYMVYNELTKVRYID--DOGKTN-YFSGQEKQQIFNDLFK--QKR-KVKK | 566 |
| WP_031669209 | 493 | SAVDFIEKMTNKDTYLPKENVLPKHSLCYQKYMVYNELTKVRYID--DOGKTN-YFSGQEKQQIFNDLFK--QKR-KVKK | 566 |
| WP_033920898 | 493 | SAVDFIEKMTNKDTYLPKENVLPKHSLCYQKYMVYNELTKIRYID--DOGKTN-YFSGQEKQQIFNDLFK--QKR-KVKK | 566 |
| AKI42028 | 496 | SAVDFIEKMTNKDTYLPKENVLPKHSLCYQKYMVYNELTKVRYID--DOGKTN-YFSGQEKQQIFNDLFK--QKR-KVKK | 569 |
| AKI50529 | 496 | SAVDFIEKMTNKDTYLPKENVLPKHSLCYQKYMVYNELTKIRYID--DOGKTN-YFSGQEKQQIFNDLFK--QKR-KVKK | 569 |
| EFR83390 | 1 | ---------------------------------------------------------------IFNDLFK--QKR-KVKK | 14 |
| WP_046323366 | 493 | SAIDFIEKMTNKDTYLPKENVLPKHSMCYQKYMVYNELTKIRYTD--DQGKTH-YFSGQEKQQIFNDLFK--QKR-KVKK | 566 |
| AKE81011 | 503 | SAQSFIERMTNFDKNLPNEKVLPKHSLLYEYFTVYNELTKVKYVT--EGMRKPaFLSGEQKKAIVDLLFK--TNR-KVTV | 577 |
| CUO82355 | 503 | TAEGFIKRMRSYCTYFPDEEVLPKNSLIVSKYEVYNELNKIRVDD--------kLLEVDVKNDIYNELFM--KNK-TVTE | 571 |
| WP_033162887 | 504 | TAEGFIERMKNTGTYFPDEPVMAKNSLTVSKFEVLNELNKIRING--------kLIAVETKKELLSDLFM--KNK-TITD | 572 |
| AGZ01981 | 520 | SAQSFIERMTNFDKNLPNEKVLPKHSLLYEYFTVYNELTKVKYVT--EGMRKPaFLSGEQKKAIVDLLFK--TNR-KVTV | 594 |
| AKA60242 | 487 | SAQSFIERMTNFDKNLPNEKVLPKHSLLYEYFTVYNELTKVKYVT--EGMRKPaFLSGEQKKAIVDLLFK--TNR-KVTV | 561 |
| AKS40380 | 487 | SAQSFIERMTNFDKNLPNEKVLPKHSLLYEYFTVYNELTKVKYVT--EGMRKPaFLSGEQKKAIVDLLFK--TNR-KVTV | 561 |
| 4UN5_B | 491 | SAQSFIERMTNFDKNLPNEKVLPKHSLLYEYFTVYNELTKVKYVT--EGMRKPaFLSGEQKKAIVDLLFK--TNR-KVTV | 565 |
| WP_010922251 | 562 | KOLKEDYFKK--IECFDSVEISGVEDR---FNASLGTYHDLLKIIKDK DELDNEENEDILEDIVLTLTLFEDREMIE | 633 |
| WP_039695303 | 564 | EKLLNYLNKE--FPEYRIKDLIGLDKEnkSFNASLGTYHDLKKIL-DK AFLDDKVNEEVIEDIIKTLTLFEDKDMIH | 637 |
| WP_045635197 | 562 | KDIIHYLHN---VDGYDGIELKGIEKQ---FNASLSTYHDLLKIIKDK EFMDDAKNEAILENIVHTLTIFEDREMIK | 632 |
| 5AXW_A | 300 | KQIAKEILVNe--EDIKGYRVTSTGKPe---FTNLKVYHDIKDITARK ------ENAELLDQIAKILTIYQSSEDIQ | 368 |
| WP_009880683 | 246 | KOLKEDYFKK--IECFDSVEISGVEDR---FNASLGTYHDLLKIIKDK DFLDNEENEDILEDIVLTLTLFEDREMIE | 317 |
| WP_010922251 | 562 | KOLKEDYFKK--IECFDSVEISGVEDR---FNASLGTYHDLLKIIKDK DELDNEENEDILEDIVLTLTLFEDREMIE | 633 |
| WP_011054416 | 562 | KOLKEDYFKK--IECFDSVEISGVEDR---FNASLGTYHDLLKIIKDK DFLDNEENEDILEDIVLTLTLFEDREMIE | 633 |
| WP_011284745 | 562 | KOLKEDYFKK--IECFDSVEISGVEDR---FNASLGTYHDLLKIIKDK DFLDNEENEDILEDIVLTLTLFEDREMIE | 633 |
| WP_011285506 | 562 | KOLKEDYFKK--IECFDSVEISGVEDR---FNASLGTYHDLLKIIKDK DFLDNEENEDILEDIVLTLTLFEDREMIE | 633 |
| WP_011527619 | 562 | KOLKEDYFKK--IECFDSVEISGVEDR---FNASLGTYHDLLKIIKDK DFLDNEENEDILEDIVLTLTLFEDREMIE | 633 |
| WP_012560673 | 562 | KOLKEDYFKK--IECFDSVEISGVEDR---FNASLGTYHDLLKIIKDK DFLDNEENEDILEDIVLTLTLFEDREMIE | 633 |
| WP_014407541 | 562 | KOLKEDYFKK--IECFDSVEISGVEDR---FNASLGAYHDLLKIIKDK DFLDNEENEDILEDIVLTLTLFEDRGMIE | 633 |
| WP_020905136 | 562 | KOLKEDYFKK--IECFDSVEISGVEDR---FNASLGTYHDLLKIIKDK DFLDNEENEDILEDIVLTLTLFEDREMIE | 633 |
| WP_023080005 | 562 | KOLKEDYFKK--IECFDSVEISGVEDR---FNTSLGTYHDLLKIIKDK DFLDNEENEDILEDIVLTLTLFEDKEMIE | 633 |
| WP_023610282 | 562 | KOLKEDYFKK--IECFDSVEISGVEDR---FNTSLGTYHDLLKIIKDK DFLDNEENEDILEDIVLTLTLFEDKEMIE | 633 |
| WP_030125963 | 562 | KOLKEDYFKK--IECFDSVEISGVEDR---FNASLGTYHDLLKIIKDK DELDNEENEDILEDIVLTLTLFEDREMIE | 633 |
| WP_030126706 | 562 | KOLKEDYFKK--IECFDSVEISGVEDR---FNASLGTYHDLLKIIKDK DELDNEENEDILEDIVLTLTLFEDREMIE | 633 |
| WP_031488318 | 562 | KOLKEDYFKK--IECFDSVEISGVEDR---FNASLGTYHDLLKIIKDK DFLDNEENEDILEDIVLTLTLFEDREMIE | 633 |
| WP_032460140 | 562 | KOLKEDYFKK--IECFDSVEISGVEDR---FNASLGTYHDLLKIIKDK DELDNEENEDILEDIVLTLTLFEDREMIE | 633 |
| WP_032461047 | 562 | KQLKEDYFKK--IECFDSVEISGVEDR---FNASLGTYHDLLKIIKDK DELDNEENEDILEDIVLTLTLFEDREMIE | 633 |
| WP_032462016 | 562 | KOLKEDYFKK--IECFDSVEISGVEDR---FNASLGTYHDLLKIIKDK DFLDNEENEDILEDIVLTLTLFEDREMIE | 633 |
| WP_032462936 | 562 | KOLKEDYFKK--IECFDSVEISGVEDR---FNASLGTYHDLLKIIKDK DELDNEENEDILEDIVLTLTLFEDKEMIE | 633 |
| WP_032464890 | 562 | KOLKEDYFKK--IECFDSVEISGVEDR---FNASLGTYHDLLKIIKDK DFLDNEENEDILEDIVLTLTLFEDREMIE | 633 |
| WP_033888930 | 387 | KOLKEDYFKK--IECFDSVEISGVEDR---FNASLGTYHDLLKIIKDK DFLDNEENEDILEDIVLTLTLFEDREMIE | 458 |
| WP_038431314 | 562 | KOLKEDYFKK--IECFDSVEISGVEDR---FNASLGTYHDLLKIIKDK DFLDNEENEDILEDIVLTLTLFEDREMIE | 633 |
| WP_038432938 | 562 | KOLKEDYFKK--IECFDSVEISGVEDR---FNTSLGTYHDLLKIIKDK DELDNEENEDILEDIVLTLTLFEDKEMIE | 633 |
| WP_038434062 | 562 | KOLKEDYFKK--IECFDSVEISGVEDR---FNASLGTYHDLLKIIKDK DFLDNEENEDILEDIVLTLTLFEDREMIE | 633 |
| BAQ51233 | 473 | KOLKEDYFKK--IECFDSVEISGVEDR---FNASLGTYHDLLKIIKDK DFLDNEENEDILEDIVLTLTLFEDREMIE | 544 |
| KGE60162 | | ------------------------------------------------ ----------------------------- | |
| KGE60856 | | ------------------------------------------------ ----------------------------- | |
| WP_002989955 | 562 | KOLKEDYFKK--IECFDSVEISGVEDR---FNASLGTYHDLLKIIKDK DFLDNEENEDILEDIVLTLTLFEDREMIE | 633 |
| WP_003030002 | 561 | DKLLNYLNKE--FEEFRIVNLTGLDKEnkAFNSSLGTYHDLRKIL-DK SFLDDKANEKTIEDIIQTLTLFEDREMIR | 634 |
| WP_003065552 | 564 | EKLLNYLNKE--FPEYRIKDLIGLDKEnkSFNASLGTYHDLKKIL-DK AFLDDKVNEEVIEDIIKTLTLFEDKDMIH | 637 |
| WP_001040076 | 563 | KDIISFLNK---VDGYEGIAIKGIEKQ---FNASLSTYHDLKKIL-GK DFLDNTDNELILEDIVQTLTLFEDREMIK | 632 |
| WP_001040078 | 562 | KKLLDFLAKE--YEEFRIVDVIGLDKEnkAFNASLGTYHDLEKIL-DK DFLDNPDNESILEDIVOTLTLFEDREMIK | 635 |
| WP_001040080 | 562 | KKLLDFLAKE--YEEFRIVDVIGLDKEnkAFNASLGTYHDLEKIL-DK DFLDNPDNESILEDIVQTLTLFEDREMIK | 635 |
| WP_001040081 | 562 | KKLLDFLAKE--YEEFRIVDVIGLDKEnkAFNASLGTYHDLEKIL-DK DELDNPDNESILEDIVOTLTLFEDREMIK | 635 |
| WP_001040083 | 562 | KKLLDFLAKE--YEEFRIVDVIGLDKEnkAFNASLGTYHDLEKIL-DK DELDNPDNESILEDIIQTLTLFEDREMIK | 635 |
| WP_001040085 | 562 | KKLLDFLAKE--YEEFRIVDVIGLDKEnkAFNASLGTYHDLEKIL-DK DFLDNPDNESILEDIVQTLTLFEDREMIK | 635 |
| WP_001040087 | 562 | KKLLDFLAKE--YEEFRIVDVIGLDKEnkAFNASLGTYHDLEKIL-DK DELDNPDNESILEDIVQTLTLFEDREMIK | 635 |
| WP_001040088 | 562 | KKLLDFLAKE--YEEFRIVDVIGLDKEnkAFNASLGTYHDLEKIL-DK DFLDNPDNESILEDIVQTLTLFEDREMIK | 635 |
| WP_001040089 | 562 | KKLLDFLAKE--YEEFRIVDVIGLDKEnkAFNASLGTYHDLEKIL-DK DFLDNPDNESILEDIVOTLTLFEDREMIK | 635 |
| WP_001040090 | 562 | KKLLDFLAKE--YEEFRIVDVIGLDKEnkAFNASLGTYHDLEKIL-DK DFLDNPDNESILEDIVQTLTLFEDREMIK | 635 |
| WP_001040091 | 562 | KKLLDFLAKE--YEEFRIVDVIGLDKEnkAFNASLGTYHDLEKIL-DK DFLDNPDNESILEDIVQTLTLFEDREMIK | 635 |
| WP_001040092 | 562 | KQLLDFLAKE--FEEFRIVDVTGLDKEnkAFNASLGTYHDLKKIL-DK DFLDNPDNESILEDIVQTITLFEDREMIK | 635 |
| WP_001040094 | 563 | KDIISFLNK---VDGYEGIAIKGIEKQ---FNASLSTYHDLKKIL-GK DFLDNTDNELILEDIVQTLTLFEDREMIR | 632 |
| WP_001040095 | 563 | KDIISFLNK---VDGYEGIAIKGIEKQ---FNASLSTYHDLKKIL-GK DFLDNTDNELILEDIVOTLTLFEDREMIR | 632 |
| WP_001040096 | 563 | KDIISELNK---VDGYEGIAIKGIEKQ---FNASLSTYHDLKKIL-GK DFLDNTDNELILEDIVOTLTLFEDREMIR | 632 |
| WP_001040097 | 563 | KDIISFLNK---VDGYEGIAIKGIEKQ---FNASLSTYHDLKKIL-GK DFLDNTDNELILEDIVQTLTLFEDREMIR | 632 |
| WP_001040098 | 563 | KDIISFLNK---VDGYEGIAIKGIEKQ---FNASLSTYHDLKKIL-GK DFLDNTDNELILEDIVOTLTLFEDREMIR | 632 |
| WP_001040099 | 563 | KDIISFLNK---VDGYEGIAIKGIEKQ---FNASLSTYHDLKKIL-GK DFLDNTDNELILEDIVQTLTLFEDREMIR | 632 |
| WP_001040100 | 563 | KDIISFLNK---VDGYEGIAIKGIEKQ---FNASLSTYHDLKKIL-GK DFLDNTDNELILEDIVQTLTLFEDREMIR | 632 |
| WP_001040104 | 562 | KKLLDFLAKE--YEEFRIVDVIGLDKEnkAFNASLGTYHDLEKIL-DK DFLDNPDNESILEDIVQTLTLFEDREMIK | 635 |
| WP_001040105 | 562 | KKLLDFLAKE--YEEFRIVDVIGLDKEnkAFNASLGTYHDLEKIL-DK DFLDNPDNESILEDIVOTLTLFEDREMIK | 635 |
| WP_001040106 | 562 | KKLLDFLAKE--YEEFRIVDVIGLDKEnkAFNASLGTYHDLKKIL-DK DFLDNPDNESILEDIVQTLTLFEDREMIK | 635 |
| WP_001040107 | 562 | KKLLDFLAKE--YEEFRIVDVIGLDKEnkAFNASLGTYHDLKKIL-DK DELDNPDNESILEDIVQTLTLFEDREMIK | 635 |
| WP_001040108 | 562 | KKLLDFLAKE--YEEFRIVDVIGLDKEnkAFNASLGTYHDLKKIL-DK DELDNPDNESILEDIVOTLTLFEDREMIK | 635 |
| WP_001040109 | 562 | KKLLDFLAKE--YEEFRIVDVIGLDKEnkAFNASLGTYHDLKKIL-DK DELDNPDNESILEDIVQTLTLFEDREMIK | 635 |
| WP_001040110 | 562 | KKLLDFLAKE--YEEFRIVDVIGLDKEnkAFNASLGTYHDLKKIL-DK DFLDNPDNESILEDIVQTLTLFEDREMIK | 635 |
| WP_015058523 | 562 | KOLLDFLAKE--FEEFRIVDVTGLDKEnkAFNASLGTYHDLKKIL-DK DFLDNPDNESILEDIVQTITLFEDREMIK | 635 |
| WP_017643650 | 563 | KDIISFLNK---VDGYEGIAIKGIEKQ---FNASLSTYHDLKKIL-GK DELDNTDNELILEDIVOTLTLFEDREMIR | 632 |
| WP_017647151 | 562 | KKLLDFLAKE--YEEFRIVDVIGLDKEnkAFNASLGTYHDLKKIL-DK DELDNPDNESILEDIVOTLTLFEDREMIK | 635 |
| WP_017648376 | 562 | KKLLDFLAKE--YEEFRIVDVIGLDKEnkAFNASLGTYHDLKKIL-DK DELDNPDNESILEDIVOTLTLFEDREMIK | 635 |
| WP_017649527 | 562 | KKLLDFLAKE--YEEFRIVDVIGLDKEnkAFNASLGTYHDLEKIL-DK DFLDNPDNESILEDIVOTLTLFEDREMIK | 635 |
| WP_017771611 | 562 | KKLLDFLAKE--YEEFRIVDVIGLDKEnkAFNASLGTYHDLKKIL-DK DFLDNPDNESILEDIVOTLTLFEDREMIK | 635 |
| WP_017771984 | 562 | KKLLDFLAKE--YEEFRIVDVIGLDKEnkAFNASLGTYHDLEKIL-DK DFLDNPDNESILEDIVOTLTLFEDREMIK | 635 |
| CFQ25032 | 562 | KKLLDFLAKE--YEEFRIVDVIGLDKEnkAFNASLGTYHDLEKIL-DK DFLDNPDNESILEDIVQTLTLFEDREMIK | 635 |
| CFV16040 | 562 | KKLLDFLAKE--YEEFRIVDVIGLDKEnkAFNASLGTYHDLEKIL-DK DFLDNPDNESILEDIVQTLTLFEDREMIK | 635 |
| KLJ37842 | 562 | KKLLDFLAKE--YEEFRIVDVIGLDKEnkAFNASLGTYHDLEKIL-DK DFLDNPDNESILEDIVOTLTLFEDREMIK | 635 |
| KLJ72361 | 562 | KKLLDFLAKE--YEEFRIVDVIGLDKEnkAFNASLGTYHDLEKIL-DK DFLDNPDNESILEDIVOTLTLFEDREMIK | 635 |
| KLL20707 | 562 | KKLLDFLAKE--YEEFRIVDVIGLDKEnkAFNASLGTYHDLEKIL-DK DFLDNPDNESILEDIVOTLTLFEDREMIK | 635 |
| KLL42645 | 562 | KKLLDFLAKE--YEEFRIVDVIGLDKEnkAFNASLGTYHDLKKIL-DK DFLDNPDNESILEDIVOTLTLFEDREMIK | 635 |
| WP_047207273 | 562 | KKLLDFLAKE--YEEFRIVDVIGLDKEnkAFNASLGTYHDLEKIL-DK DFLDNPDNESILEDIVOTLTLFEDREMIK | 635 |
| WP_047209694 | 563 | KDIISFLNK---VDGYEGIAIKGIEKQ---FNASLSTYHDLKKIL-GK DFLDNTDNELILEDIVQTLTLFEDREMIR | 632 |
| WP_050198062 | 562 | KKLLDFLAKE--YEEFRIVDVIGLDKEnkAFNASLGTYHDLEKIL-DK DFLDNPDNESILEDIVQTLTLFEDREMIK | 635 |
| WP_050201642 | 562 | KKLLDFLAKE--YEEFRIVDVIGLDKEnkAFNASLGTYHDLEKIL-DK DFLDNPDNESILEDIVQTLTLFEDREMIK | 635 |
| WP_050204027 | 562 | KKLLDFLAKE--YEEFRIVDVIGLDKEnkAFNASLGTYHDLKKIL-DK DFLDNPDNESILEDIVOTLTLFEDREMIK | 635 |
| WP_050881965 | 562 | KKLLDFLAKE--YEEFRIVDVIGLDKEnkAFNASLGTYHDLEKIL-DK DFLDNPDNESILEDIVQTLTLFEDREMIK | 635 |
| WP_050886065 | 562 | KKLLDFLAKE--YEEFRIVDVIGLDKEnkAFNASLGTYHDLEKIL-DK DFLDNPDNESILEDIVQTLTLFEDREMIK | 635 |
| AHN30376 | 562 | KOLLDFLAKE--FEEFRIVDVTGLDKEnkAFNASLGTYHDLKKIL-DK DFLDNPDNESILEDIVQTITLFEDREMIK | 635 |
| EAO78426 | 562 | KKLLDFLAKE--YEEFRIVDVIGLDKEnkAFNASLGTYHDLEKIL-DK DFLDNPDNESILEDIVQTLTLFEDREMIK | 635 |
| CCW42055 | 562 | KQLLDFLAKE--FEEFRIVDVTGLDKEnkAFNASLGTYHDLEKIL-GK DFLDNPDNESILEDIVQTLTLFEDREMIK | 635 |
| WP_003041502 | 561 | DKLLNYLNKE--FEEFRIVNLTGLDKEnkVFNSSLGTYHDLRKIL-NK SFLDNKENAQIIEDIIQTLTLFEDREMIR | 634 |
| WP_037593752 | 562 | DKLLNYLNKE--FEEFRIVNLTGLDKEnkAFNSSLGTYHDLRKIL-DK SFLDDKANEKTIEDIIQTLTLFEDREMIR | 635 |
| WP_049516684 | 562 | DKLLNYLNKE--FEEFRIVNLTGLDKEnkAFNASLGTYHDLRKIL-DK SFLDDKVNEKIIEDIIQTLTLFEDREMIR | 635 |
| GAD46167 | 561 | DKLLNYLNKE--FEEFRIVNLTGLDKEnkAFNSSLGTYHDLRKIL-DK SFLDDKANEKTIEDIIQTLTLFEDREMIR | 634 |
| WP_018363470 | 562 | EKLLNYLDKE--FPEYRIQDLVGLDKEnkSFNASLGTYHDLKKIL-DK SFLDDKVNEEVIEDIIKTLTLFEDREMIQ | 635 |
| WP_003043819 | 572 | KOLKEDYFKK--IECFDSVEIIGVEDR---FNASLGTYHDLLKIIKDK DELDNEENEDILEDIVLTLTLFEDREMIE | 643 |
| WP_006269658 | 561 | DKLLNYLNKE--FEEFRIVNLTGLDKEnkAFNSSLGTYHDLRKIL-DK SFLDDKANEKTIEDIIQTLTLFEDREMIR | 634 |
| WP_048800889 | 561 | DKLLNYLDKE--FDEFRIVDLTGLDKEnkAFNASLGTYHDLRKIL-DK SFLDDKANEKTIEDIIQTLTLFEDREMIR | 634 |
| WP_012767106 | 562 | KQLKEDYFKK--IECFDSVEISGVEDR---FNASLGTYHDLLKIIKDK DELDNEENEDILEDIVLTLTLFEDKEMIE | 633 |
| WP_014612333 | 562 | KOLKEDYFKK--IECFDSVEISGVEDR---FNASLGTYHDLLKIIKDK DFLDNEENEDILEDIVLTLTLFEDKEMIE | 633 |
| WP_015017095 | 562 | KOLKEDYFKK--IECFDSVEISGVEDR---FNASLGTYHDLLKIIKDK DFLDNEENEDILEDIVLTLTLFEDKEMIE | 633 |
| WP_015057649 | 562 | KOLKEDYFKK--IECFDSVEISGVEDR---FNASLGTYHDLLKIIKDK DELDNEENEDILEDIVLTLTLFEDKEMIE | 633 |
| WP_048327215 | 562 | KOLKEDYFKK--IECFDSVEISGVEDR---FNASLGTYHDLLKIIKDK DELDNEENEDILEDIVLTLTLFEDKEMIE | 633 |
| WP_049519324 | 562 | KOLKEDYFKK--IECFDSVEISGVEDR---FNASLGTYHDLLKIIKDK DELDNEENEDILEDIVLTLTLFEDKEMIE | 633 |
| WP_012515931 | 562 | KQLKENYFKK--IECWDSVEITGVEDS---FNASLGTYHDLLKIIQDK DFLDNPDNQKIIEDIILTLTLFEDKKMIS | 633 |
| WP_021320964 | 562 | KOLKENYFKK--IECWDSVEITGVEDS---FNASLGTYHDLLKIIQDK DFLDNPDNOKIIEDIILTLTLFEDKKMIS | 633 |
| WP_037581760 | 562 | KOLKENYFKK--IECWDSVEITGVEDS---FNASLGTYHDLLKIIQDK DFLDNPDNOKIIEDIILTLTLFEDKKMIS | 633 |
| WP_004232481 | 561 | AKLLSYLNNE--FEEFRINDLIGLDKDskSFNASLGTYHDLKKIL-DK SFLDDKTNEQIIEDIVLTLTLFEDRDMIH | 634 |
| WP_009854540 | 562 | EKLLNYLNKE--FPEYRIKDLIGLDKEnkSFNASLGTYHDLKKIL-DK AFLDDKVNEEVIEDIIKTLTLFEDKDMIH | 635 |
| WP_012962174 | 562 | DKFLNYLNKE--FPEYRIQDLIGLDKEnkSFNASLGTYHDLKKIL-DK SFLDDKTNETIIEDIIQTLTLFEDRDMIR | 635 |
| WP_039695303 | 564 | EKLLNYLNKE--FPEYRIKDLIGLDKEnkSFNASLGTYHDLKKIL-DK AFLDDKVNEEVIEDIIKTLTLFEDKDMIH | 637 |
| WP_014334983 | 561 | AKLLSYLNNE--FEEFRINDLIGLDKDskSFNASLGTYHDLKKIL-DK SFLDDKINGQIIEDIVLTLTLFEDRDMIH | 634 |
| WP_003099269 | 562 | KOLKEEYFSK--MKCFHTVTILGVEDR---FNASLGTYHDLLKIFKDK AFLDDEANQDILEEIVWTLTLFEDQAMIE | 633 |
| AHY15608 | 562 | KQLKEEYFSK--MKCFHTVTILGVEDR---FNASLGTYHDLLKIFKDK AFLDDEANQDILEEIVWTLTLFEDQAMIE | 633 |
| AHY17476 | 562 | KQLKEEYFSK--MKCFHTVTILGVEDR---FNASLGTYHDLLKIFKDK AFLDDEANQDILEEIVWTLTLFEDQAMIE | 633 |
| ESR09100 | | ------------------------------------------------ ----------------------------- | |
| AGM98575 | 562 | KOLKEEYFSK--MKCFHTVTILGVEDR---FNASLGTYHDLLKIFKDK AFLDDEANQDILEEIVWTLTLFEDQAMIE | 633 |
| ALF27331 | 561 | DKLMDFLEKE--FDEFRIVDLTGLDKEnkAFNASYGTYHDLRKIL-DK DELDNSKNEKILEDIVLTLTLFEDREMIR | 634 |
| WP_018372492 | 574 | RKLKDFIEKElgYGYIDIDNIKGVEEQ---FNASYTTYQDLLKIIGDK EFLDNEENKDLLEEIIYILTVFEDRKMIE | 647 |
| WP_045618028 | 563 | KDIIQYLHN---VDSYDGIELKGIEKQ---FNASLSTYHDLLKIIKDK EFMDDSKNEAILENIVHTLTIFEDREMIK | 633 |
| WP_045635197 | 562 | KDIIHYLHN---VDGYDGIELKGIEKQ---FNASLSTYHDLLKIIKDK EFMDDAKNEAILENIVHTLTIFEDREMIK | 632 |
| WP_002263549 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002263887 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002264920 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002269043 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002269448 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002271977 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002272766 | 561 | DKLMDFLEKE--FDEFRIVDLTGLDKEnkVFNASYGTYHDLCKIL-DK DELDNSKNEKILEDIVLTLILFEDREMIR | 634 |
| WP_002273241 | 561 | DKLMDFLEKE--FDEFRIVDLTGLDKEnkVENASYGTYHDLCKIL-DK DFLDNSKNEKILEDIVLTLTLFEDREMIR | 634 |
| WP_002275430 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002276448 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002277050 | 561 | KKLRTFLDKN--FDEFRIVDIQGLDKEteTFNASYATYQDLLKVIKDK VEMDNPENAEILENIVLTLTLFEDREMIK | 635 |
| WP_002277364 | 561 | DKLMDFLEKE--FDEFRIVDLTGLDKEnkAFNASYGTYHDLRKIL-DK DELDNSKNEKILEDIVLTLTLFEDREMIR | 634 |
| WP_002279025 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002279859 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002280230 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002281696 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002282247 | 561 | KKLRTFLDKN--FDEFRIVDIQGLDKEteTFNASYATYQDLLKVIKDK VEMDNPENAEILENIVLTLTLFEDREMIK | 635 |
| WP_002282906 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002283846 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002287255 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002288990 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002289641 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002290427 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002295753 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002296423 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002304487 | 571 | DKLLNYLNKE--FEEFRIVNLTGLDKEnkVFNSSLGTYHDLRKIL-NK SFLDNKENEQIIEDIIQTLTLFEDREMIR | 644 |
| WP_002305844 | 561 | DKLMDFLEKE--FDEFRIVDLTGLDKEnkVFNASYGTYHDLCKIL-DK DFLDNSKNEKILEDIVLTLTLFEDREMIR | 634 |
| WP_002307203 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002310390 | 561 | DKLMDFLEKE--FDEFRIVDLTGLDKEnkAFNASYGTYHDLRKIL-DK DFLDNSKNEKILEDIVLTLTLFEDREMIR | 634 |
| WP_002352408 | 561 | DKLMDFLEKE--FDEFRIVDLTGLDKEnkAFNASYGTYHDLRKIL-DK DFLDNSKNEKILEDIVLTLTLFEDREMIR | 634 |
| WP_012997688 | 561 | DKLMDFLEKE--FDEFRIVDLTGLDKEnkAFNASYGTYHDLRKIL-DK DFLDNSKNEKILEDIVLTLTLFEDREMIR | 634 |
| WP_014677909 | 561 | DKLMDFLEKE--FDEFRIVDLTGLDKEnkAFNASYGTYHDLRKIL-DK DFLDNSKNEKILEDIVLILTLFEDREMIR | 634 |
| WP_019312892 | 561 | DKLMDFLEKE--FDEFRIVDLTGLDKEnkAFNASYGTYHDLRKIL-DK DFLDNSKNEKILEDIVLTLTLFEDREMIR | 634 |
| WP_019313659 | 561 | DKLMDFLEKE--FDEFRIVDLTGLDKEnkVFNASYGTYHDLCKIL-DK DFLDNSKNEKILEDIVLTLTLFEDREMIR | 634 |
| WP_019314093 | 561 | DKLMDFLEKE--FDEFRIVDLTGLDKEnkAFNASYGTYHDLRKIL-DK DFLDNSKNEKILEDIVLTLTLFEDREMIR | 634 |
| WP_019315370 | 561 | DKLMDFLEKE--FDEFRIVDLTGLDKEnkAFNASYGTYHDLRKIL-DK DFLDNSKNEKILEDIVLTLTLFEDREMIR | 634 |
| WP_019803776 | 561 | DKLMDFLEKE--FDEFRIVDLTGLDKEnkVFNASYGTYHDLCKIL-DK DFLDNSKNEKILEDIVLTLTLFEDREMIR | 634 |
| WP_019805234 | 561 | DKLMDFLEKE--FDEFRIVDLTGLDKEnkAFNASYGTYHDLRKIL-DK DFLDNSKNEKILEDIVLTLTLFEDREMIR | 634 |
| WP_024783594 | 561 | DKLMDFLEKE--FDEFRIVDLTGLDKEnkVFNASYGTYHDLCKIL-DK DFLDNSKNEKILEDIVLTLTLFEDREMIR | 634 |
| WP_024784288 | 561 | KKLRTFLDKN--FDEFRIVDIQGLDKEteTFNASYATYQDLLKVIKDK VFMDNPENAEILENIVLTLTLFEDREMIK | 635 |
| WP_024784666 | 561 | DKLMDFLEKE--FDEFRIVDLTGLDKEnkAFNASYGTYHDLRKIL-DK DFLDNSKNEKILEDIVLTLTLFEDREMIR | 634 |
| WP_024784894 | 561 | DKLMDFLEKE--FDEFRIVDLTGLDKEnkVFNASYGTYHDLCKIL-DK DFLDNSKNEKILEDIVLTLTLFEDREMIR | 634 |
| WP_024786433 | 561 | KKLRTFLDKN--FDEFRIVDIQGLDKEteTFNASYATYQDLLKVIKDK VFMDNPENAEILENIVLTLTLFEDREMIK | 635 |
| WP_049473442 | 561 | DKLMDFLEKE--FDEFRIVDLTGLDKEnkVFNASYGTYHDLCKIL-DK DELDNSKNEKILEDIVLTLTLFEDREMIR | 634 |
| WP_049474547 | 561 | DKLMDFLEKE--FDEFRIVDLTGLDKEnkVFNASYGTYHDLCKIL-DK DFLDNSKNEKILEDIVLTLTLFEDREMIR | 634 |
| EMC03581 | 554 | DKLMDFLEKE--FDEFRIVDLTGLDKEnkAFNASYGTYHDLRKIL-DK DFLDNSKNEKILEDIVLTLTLFEDREMIR | 627 |
| WP_000428612 | 565 | KDIIQYLHT---VDGYDGIELKGIEKQ---FNASLSTYHDLLKIIKDK EFMDDPNNEEILENIVHTLTIFEDREMIK | 635 |
| WP_000428613 | 563 | KDIIQFLHN---VDGYDGIELKGIEKQ---FNASLSTYHDLLKIIKDK EFMDDSKNEEILENIVHTLTIFEDREMIK | 633 |
| WP_049523028 | 562 | KDIIHYLHN---VDGYDGIELKGIEKH---FNSSLSTYHDLLKIIKDK EFMDDPKNEEIFENIVHTLTIFEDRVMIK | 632 |
| WP_003107102 | 531 | KOLKENYFNK--IRCLDSITISGVEDK---FNASLGTYHDLLNIIKNQ KILDDEQNQDSLEDIVLTLTLFEDEKMIA | 602 |
| WP_054279288 | 564 | KOLKEDFFSK--IECFDTVDISGVEDK---FNASLGTYHDLLKIIKDK AFLDNSENENIIEDIILTLTLFEDKEMIA | 635 |
| WP_049531101 | 563 | KDLIHYLHN---VDGYDGIELKGIEKQ---FNASLSTYHDLLKIIKDK RFMDEPKNQEILENIVHTLTIFEDREMIK | 633 |
| WP_049538452 | 563 | KDIIQYLHN---VDGYDGIELKGIEKQ---FNASLSTYHDLLKIIKDK EFMDDSKNEEILENIVHTLTIFEDREMIK | 633 |
| WP_049549711 | 563 | KDIIHYLHT---VDGYDGIELKGIEKQ---FNASLSTYHDLLKIIKDK EFMDDSKNEAILENIVHTLTIFEDREMIK | 633 |
| WP_007896501 | 565 | KDLKEKYFSQ--IEGLENVDVTGVEGA---FNASLGTYNDLLKIIKDK AFLDDEANAEILEEIVLILTLFQDEKLIE | 636 |
| EFR44625 | 517 | KDLKEKYFSQ--IEGLENVDVTGVEGA---FNASLGTYNDLLKIIKDK AFLDDEANAEILEEIVLILTLFQDEKLIE | 588 |
| WP_002897477 | 562 | KDIIHYLHN---VDGYDGIELKGIEKQ---FNANLSTYHDLLKITKDK EFMDDPKNEEILENIVHTLTIFEDREMIK | 632 |
| WP_002906454 | 562 | KDIIHYLHN---VDGYDGIELKGIEKQ---FNASLSTYHDLLKIIKDK EFMDNPKNGEILENIIHTLTIFEDREMIK | 632 |
| WP_009729476 | 563 | KDIIQFLHN---VDGYDGIELKGIEKQ---FNASLSTYHDLLKIIKDK AFMDDAKNEAILENIVHTLTIFEDREMIK | 633 |
| CQR24647 | 562 | KKILNFLDKN--FDEFRITDIQGLDNEtgNFNASYGTYHDLLKIIGDK EFMDSSDNVDVLEDIVLSLTLFEDREMIK | 636 |
| WP_000066813 | 565 | KDIIHYLHN---VDGYDGIELKGIEKQ---FNASLSTYHDLLKIIKDK AFMDDSKNEEILENIIHTLTIFEDREMIK | 635 |
| WP_009754323 | 563 | KDIIHYLHN---VDGYDGIELKGIEKQ---FNASLSTYHDLLKIIKDK EFMDNHKNQEILENIVHTLTIFEDREMIK | 633 |
| WP_044674937 | 562 | KDIKAYL-EN--SNGYAGVELKGLEEQ---FNASLPTYHDLLKILRDK AFIDAEENQEILEDIVLTLTLFEDREMIR | 632 |
| WP_044676715 | 561 | EKLMDFLGKE--FDEFRIVDLLGLDKDnkSFNASLGTYHDLKKIV-SK DLLDNPENEDILENVVLTLTLFEDREMIR | 634 |
| WP_044680361 | 561 | EKLMDFLGKE--FDEFRIVDLLGLDKDnkSFNASLGTYHDLKKIV-SK DLLDNPENEDILENVVLTLTLFEDREMIR | 634 |
| WP_044681799 | 562 | KDIKAYL-EN--SNGYAGVELKGLEEQ---FNASLPTYHDLLKILRDK AFIDAEENQEILEDIVLTLTLFEDREMIR | 632 |
| WP_049533112 | 561 | DKLLNYLGKE--FDEFRIVDLTGLDKEnkVFNSSLGTYHDLRKIL-DK SFLDNKENEQIIEDIIQTLTLFEDREMIR | 634 |
| WP_029090905 | 541 | TSLKKWLAEH---EHMTVSVVQGTQKEt-EFATSLQAFHRFVKIF-DR ETVSNPANEEMFEKIIYWSTVFEDKKIMR | 612 |
| WP_006506696 | 568 | KKLKNWLVNNqcCS--KDAEIKGFQKEn-QFSTSLTPWIDFTNIFGKI --DQSNFDLIENIIYDLTVFEDKKIMK | 637 |
| AIT42264 | 562 | KOLKEDYFKK--IECFDSVEISGVEDR---FNASLGTYHDLLKIIKDK DFLDNEENEDILEDIVLTLTLFEDREMIE | 633 |
| WP_034440723 | 568 | NOLVKYIENK---EQIIAPEIKGIEDS---FNSNYSTYIDLSKIPDMK --LLEKDEDEILEEIIKILTIFEDRKMRK | 637 |
| AKQ21048 | 562 | KQLKEDYFKK--IECFDSVEISGVEDR---FNASLGTYHDLLKIIKDK DELDNEENEDILEDIVLTLTLFEDREMIE | 633 |
| WP_004636532 | 563 | KDIANYLEQ---YGYVDGTDIKGVEDK---FNASLSTYNDLAKIDGAK AYLDDPEYADVWEDIIKILTIFEDKAMRK | 633 |
| WP_002364836 | 570 | KDIIQFYRNE--YN-TEIVTLSGLEED--QFNASFSTYQDLLKCGLTR AELDHPDNAEKLEDIIKILTIFEDRORIR | 641 |
| WP_016631044 | 521 | KDIIQFYRNE--YN-TEIVTLSGLEED--QFNASFSTYQDLLKCGLTR AELDHPDNAEKLEDIIKILTIFEDRORIR | 592 |
| EMS75795 | 306 | KKLQQFLSAN--YN-IEDAEILGVDKA---FNSSYATYHDELDLAKPN ELLEQPEMNAMFEDIVKILTIFEDREMIR | 381 |
| WP_002373311 | 570 | KDIIQFYRNE--YN-TEIVTLSGLEED--QFNASFSTYQDLLKCGLTR AELDHPDNAEKLEDIIKILTIFEDRORIR | 641 |
| WP_002378009 | 570 | KDIIQFYRNE--YN-TEIVTLSGLEED--QFNASFSTYQDLLKCGLTR AELDHPDNAEKLEDIIKILTIFEDRORIR | 641 |
| WP_002407324 | 570 | KDIIQFYRNE--YN-TEIVTLSGLEED--QFNASFSTYQDLLKCGLTR AELDHPDNAEKLEDIIKILTIFEDRORIR | 641 |
| WP_002413717 | 570 | KDIIQFYRNE--YN-TEIVTLSGLEED--QFNASFSTYQDLLKCGLTR AELDHPDNAEKLEDIIKILTIFEDRQRIR | 641 |
| WP_010775580 | 572 | KDIIQFYRNE--YN-TEIVTLSGLEED--QFNASFSTYQDLLKCGLTR AELDHPDNAEKLEDIIKILTIFEDRORIR | 643 |
| WP_010818269 | 570 | KDIIQFYRNE--YN-TEIVTLSGLEED--QFNASFSTYQDLLKCGLTR AELDHPDNAEKLEDIIKILTIFEDRQRIR | 641 |
| WP_010824395 | 570 | KDIIQFYRNE--YN-TEIVTLSGLEED--QFNASFSTYQDLLKCGLTR AELDHPDNAEKLEDIIKILTIFEDRORIR | 641 |
| WP_016622645 | 570 | KDIIQFYRNE--YN-TEIVTLSGLEED--QFNASFSTYQDLLKCGLTR AELDHPDNAEKLEDIIKILTIFEDRQRIR | 641 |
| WP_033624816 | 570 | KDIIQFYRNE--YN-TEIVTLSGLEED--QFNASFSTYQDLLKCGLTR AELDHPDNAEKLEDIIKILTIFEDRQRIR | 641 |
| WP_033625576 | 570 | KDIIQFYRNE--YN-TEIVTLSGLEED--QFNASFSTYQDLLKCGLTR AELDHPDNAEKLEDIIKILTIFEDRORIR | 641 |
| WP_033789179 | 570 | KDIIQFYRNE--YN-TEIVTLSGLEED--QFNASFSTYQDLLKCGLTR AELDHPDNAEKLEDIIKILTIFEDRORIR | 641 |
| WP_002310644 | 567 | KDLQEFLYLK--YD-IKHAELSGIEKA---FNASYTTYHDFLTMSENK QWLEDPELASMFEEIIKTLTVFEDREMIK | 641 |
| WP_002312694 | 568 | KDLQEFLYLK--YD-IKHAELSGIEKA---FNASYTTYHDFLTMSENK QWLEDPELASMFEEIIKTLTVFEDREMIK | 642 |
| WP_002314015 | 568 | KDLQEFLYLK--YD-IKHAELSGIEKA---FNASYTTYHDFLTMSENK QWLEDPELASMFEEIIKTLTVFEDREMIK | 642 |
| WP_002320716 | 568 | KDLQEFLYLK--YD-IKHAELSGIEKA---FNASYTTYHDFLTMSENK QWLEDPELASMFEEIIKTLTVFEDREMIK | 642 |
| WP_002330729 | 567 | KDLQEFLYLK--YD-IKHAELSGIEKA---FNASYTTYHDFLTMSENK QWLEDPELASMFEEIIKTLTVFEDREMIK | 641 |
| WP_002335161 | 568 | KDLQEFLYLK--YD-IKHAELSGIEKA---FNASYTTYHDFLTMSENK QWLEDPELASMFEEIIKTLTVFEDREMIK | 642 |
| WP_002345439 | 568 | KDLQEFLYLK--YD-IKHAELSGIEKA---FNASYTTYHDFLTMSENK QWLEDPELASMFEEIIKTLTVFEDREMIK | 642 |
| WP_034867970 | 562 | KKLONFLYTH--YH-IENAQIFGIEKA---FNASYSTYHDFMKLAKTN EWLEQPEMEPIFEDIVKILTIFEDRQMIK | 637 |
| WP_047937432 | 568 | KDLQEFLYLK--YD-IKHAELSGIEKA---FNASYTTYHDFLTMSENK QWLEDPELASMFEEIIKTLTVFEDREMIK | 642 |
| WP_010720994 | 562 | KKLONFLYTH--YH-IENAQIFGIEKA---FNASYSTYHDFMKLAKTN EWLEQPEMEPIFEDIVKILTIFEDRQMIK | 637 |
| WP_010737004 | 562 | KKLONFLYTH--YH-IENAQIFGIEKA---FNASYSTYHDFMKLAKTN EWLEQPEMEPIFEDIVKILTIFEDRQMIK | 637 |
| WP_034700478 | 562 | KKLONFLYTH--YH-IENAQIFGIEKA---FNASYSTYHDFMKLAKTN EWLEQPEMEPIFEDIVKILTIFEDROMIK | 637 |
| WP_007209003 | 564 | KKLENYLRIE1---SISSPSVKGIEEQ---FNANFGTYLDLKKEDELH PYLDDEKYQDTLEEVIKVLTVFEDRSMIQ | 634 |
| WP_023519017 | 560 | KOLRKFLELN--EQ-IDSTEIKGIETS---FNASYSTYHDLLKLS--- TLLDDPDMTTMFEEIIKILTIFEDREMIR | 631 |
| WP_010770040 | 564 | KLLEKFLSNE--FG-LVDVAIKGIE-T--SFNAGYGTYHDELKIGITR EQLDKEENSETLEEIVKILTVFEDRKMIR | 634 |
| WP_048604708 | 560 | KDLSNFLRNE--YN-LDDVIIDGIE-N--KFNASENTYHDFLKLKIDP KVLDDPANEPMFEEIVKILTIFEDRKMLR | 630 |
| WP_010750235 | 561 | KKLQHFLSAN--YN-IEDAEILGVDKV---FNSSYATYHDFLELAKPY ELLEQPEMEEMFEDIVKIITIFEDREMVR | 636 |
| AII16583 | 601 | KOLKEDYFKK--IECFDSVEISGVEDR---FNASLGTYHDLLKIIKDK DELDNEENEDILEDIVLTLTLFEDREMIE | 672 |
| WP_029073316 | 576 | NAMKKWLVKNqyFSNTDDIKIEGFQKEn-ACSTSLTPWIDFTKIFGEI ----NNSNYELIEKIIYDVTVFEDKKILR | 647 |
| WP_031589969 | 576 | NAMKKWLVKNqyFSNTDDIKIEGFQKEn-ACSTSLTPWIDFTKIFGKI ----NESNYDFIEKIIYDVTVFEDKKILR | 647 |
| KDA45870 | 558 | KMVIKHLKVV--MPAIRIQALKGLDNGk--FNASYGTYKDLVDMGVAP ELLNDEVNSEKWEDIIKTLTIFEGRKLIK | 630 |
| WP_039099354 | 579 | KNIQDYLVSEK--RYASRPAITGLSDEnk-FNSRLSTYHDLKTIVGDA --VDDVDKQADLEKCIEWSTIFEDGKIYS | 650 |
| AKP02966 | 561 | KKLTKWLIAQg---YYKNPILIGLSQKd-EFNSTLTTYLDMKKIFGSS -FMENNKNYNQIEELIEWLTIFEDKQILN | 632 |
| WP_010991369 | 567 | KDLELFLRNM--SH-VESPTIEGLE-D--SENSSYSTYHDLLKVGIKQ EILDNPVNTEMLENIVKILTVFEDKRMIK | 637 |
| WP_033838504 | 567 | KDLELFLRNM--SH-VESPTIEGLE-D--SENSSYSTYHDLLKVGIKQ EILDNPVNTEMLENIVKILTVFEDKRMIK | 637 |
| EHN60060 | 570 | KDLELFLRNM--SH-VESPTIEGLE-D--SENSSYSTYHDLLKVGIKQ EILDNPVNTEMLENIVKILTVFEDKRMIK | 640 |
| EFR89594 | 336 | KDLELFLRNM--SH-VESPTIEGLE-D--SFNSSYSTYHDLLKVGIKQ EILDNPVNTEMLENIVKILTVFEDKRMIK | 406 |
| WP_038409211 | 567 | KDLERFLYTI--NH-IESPTIEGVE-D--AFNSSFATYHDLQKGGVTQ EILDNPLNADMLEEIVKILTVFEDKRMIK | 637 |
| EFR95520 | 186 | KDLERFLYTI--NH-IESPTIEGVE-D--AFNSSFATYHDLQKGGVTQ EILDNPLNADMLEEIVKILTVFEDKRMIK | 256 |
| WP_003723650 | 567 | KDLELFLRNI--NH-IESPTIEGLE-D--SFNASYATYHDLLKVGMKQ EILDNPLNTEMLEDIVKILTVFEDKPMIK | 637 |
| WP_003727705 | 567 | KDLELFLRNI--NH-IESPTIEGLE-D--SFNASYATYHDLLKVGLKQ EILDNPLNTEILEDIVKILTVFEDKRMIK | 637 |
| WP_003730785 | 567 | KDLELFLRNI--NH-IESPTIEGLE-D--SFNASYATYHDLLKVGLKQ EILDNPLNTEILEDIVKILTVFEDKRMIK | 637 |
| WP_003733029 | 567 | KDLELFLRNI--NQ-IESPTIEGLE-D--SFNASYATYHDLLKVGMKQ EILDNPLNTEMLEDIVKILTVFEDKRMIK | 637 |
| WP_003739838 | 567 | KDLEQFLRNM--SH-IESPTIEGLE-D--SENSSYATYHDLLKVGIKQ EVLENPLNTEMLEDIVKILTVFEDKRMIK | 637 |
| WP_014601172 | 567 | KDLELFLRNI--NH-IESPTIEGLE-D--SFNASYATYHDLLKVGMKQ EILDNPLNTEMLEDIVKILTVFEDKPMIK | 637 |
| WP_023548323 | 567 | KDLELFLRNI--NH-VESPTIEGLE-D--SFNASYATYHDLMKVGIKQ EILDNPLNTEMLEDIVKILTVFEDKRMIK | 637 |
| WP_031665337 | 567 | KDLELFLRNI--NQ-IESPTIEGLE-D--SFNASYATYHDLLKVGMKQ EILDNPLNTEMLEDIVKILTVFEDKRMIK | 637 |
| WP_031669209 | 567 | KDLELFLRNI--NQ-IESPTIEGLE-D--SFNASYATYHDLLKVGMKQ EILDNPLNTEMLEDIVKILTVFEDKRMIK | 637 |
| WP_033920898 | 567 | KDLELFLRNI--NH-VESPTIEGLE-D--SFNASYATYHDLMKVGIKQ EILDNPLNTEMLEDIVKILTVFEDKRMIK | 637 |
| AKI42028 | 570 | KDLELFLRNI--NH-IESPTIEGLE-D--SFNASYATYHDLLKVGMKQ EILDNPLNTEMLEDIVKILTVFEDKPMIK | 640 |
| AKI50529 | 570 | KDLELFLRNI--NH-VESPTIEGLE-D--SFNASYATYHDLMKVGIKQ EILDNPLNTEMLEDIVKILTVFEDKRMIK | 640 |
| EFR83390 | 15 | KDLELFLRNI--NQ-IESPTIEGLE-D--SFNASYATYHDLLKVGMKQ EILDNPLNTEMLEDIVKILTVFEDKRMIK | 85 |
| WP_046323366 | 567 | KDLELFLYNM--NH-VESPTVEGVE-D--AFNSSFTTYHDLQKVGVPQ EILDDPLNTEMLEEIIKILTVFEDKRMIN | 637 |
| AKE81011 | 578 | KOLKEDYFKK--IECFDSVEISGVEDR---FNASLGTYHDLLKIIKDK DELDNEENEDILEDIVLTLTLFEDREMIE | 649 |
| CUO82355 | 572 | KKLKNWLVNNqcCR--KDAEIKGFQKEn-QFSTSLTPWIDFTNIFGKI ----DQSNEDLIEKIIYDLTVFEDKKIMK | 641 |
| WP_033162887 | 573 | KKLKDWLVTHqyYDINEELKIEGYQKD1-QFSTSLAPWIDFTKIFGEI ----NASNYQLIEKIIYDISIFEDKKILK | 644 |
| AGZ01981 | 595 | KOLKEDYFKK--IECFDSVEISGVEDR---FNASLGTYHDLLKIIKDK DELDNEENEDILEDIVLTLTLFEDREMIE | 666 |
| AKA60242 | 562 | KOLKEDYFKK--IECFDSVEISGVEDR---FNASLGTYHDLLKIIKDK DFLDNEENEDILEDIVLTLTLFEDREMIE | 633 |
| AKS40380 | 562 | KOLKEDYFKK--IECFDSVEISGVEDR---FNASLGTYHDLLKIIKDK DELDNEENEDILEDIVLTLTLFEDREMIE | 633 |
| 4UN5_B | 566 | KOLKEDYFKK--IECFDSVEISGVEDR---FNASLGTYHDLLKIIKDK DFLDNEENEDILEDIVLTLTLFEDREMIE | 637 |
| WP_010922251 | 634 | 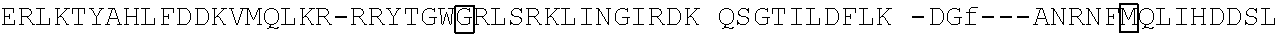 | 702 |
| WP_039695303 | 638 | ERLOKYSDIFTANQLKKLER-RHYTGWGRLSYKLINGIRNK ENNKTILDYLI DDG---SANRNFMQLINDDTL | 706 |
| WP_045635197 | 633 | QRLAQYDSLFDEKVIKALTR-RHYTGWGKLSAKLINGICDK QTGNTILDYLI DDG---KINRNFMQLINDDGL | 701 |
| 5AXW_A | 369 | EELTNLNSELTQEEIEQISN1KGYTGTHNLSLKAINLILDE ---------LW -------TNDNQIAIFNRLKL | 426 |
| WP_009880683 | 318 | ERLKTYAHLEDDKVMKOLKR-RRYTGWGRLSRKLINGIRDK QSGKTILDFLK -DGf---ANRNFMQLIHDDSL | 386 |
| WP_010922251 | 634 | ERLKTYAHLFDDKVMKOLKR-RRYTGWGRLSRKLINGIRDK QSGKTILDFLK -DGf---ANRNFMQLIHDDSL | 702 |
| WP_011054416 | 634 | ERLKTYAHLFDDKVMKOLKR-RRYTGWGRLSRKLINGIRDK QSGKTILDFLK -DGf---ANRNFMQLIHDDSL | 702 |
| WP_011284745 | 634 | ERLKTYAHLFDDKVMKOLKR-RRYTGWGRLSRKLINGIRDK QSGKTILDFLK -DGf---ANRNFMQLIHDDSL | 702 |
| WP_011285506 | 634 | ERLKTYAHLFDDKVMKOLKR-RRYTGWGRLSRKLINGIRDK QSGKTILDFLK -DGf---ANRNFMQLIHDDSL | 702 |
| WP_011527619 | 634 | ERLKTYAHLFDDKVMKOLKR-RRYTVWGRLSRKLINGIRDK QSGKTILDFLK -DGf---ANRNFMQLIHDDSL | 702 |
| WP_012560673 | 634 | ERLKTYAHLFDDKVMKOLKR-RRYTGWGRLSRKLINGIRDK QSGKTILDELK -DGf---ANRNFMQLIHDDSL | 702 |
| WP_014407541 | 634 | ERLKTYAHLFDDKVMKOLKR-RRYTGWGRLSRKLINGIRDK QSGKTILDFLK -DGf---ANRNFMQLIHDDSL | 702 |
| WP_020905136 | 634 | ERLKTYAHLFDDKVMKOLKR-RRYTGWGRLSRKLINGIRDK QSGKTILDFLK -DGf---ANRNFMQLIHDDSL | 702 |
| WP_023080005 | 634 | ERLKKYANLFDDKVMKOLKR-RHYTGWGRLSRKLINGIRDK QSGKTILDFLK -DGf---ANRNFMQLINDDSL | 702 |
| WP_023610282 | 634 | ERLKKYANLFDDKVMKOLKR-RHYTGWGRLSRKLINGIRDK QSGKTILDFLK -DGf---ANRNFMQLINDDSL | 702 |
| WP_030125963 | 634 | ERLKTYAHLFDDKVMKOLKR-RRYTGWGRLSRKLINGIRDK QSGKTILDFLK -DGf---ANRNFMQLIHDDSL | 702 |
| WP_030126706 | 634 | ERLKTYAHLFDDKVMKOLKR-RRYTGWGRLSRKLINGIRDK QSGKTILDFLK -DGf---ANRNFMQLIHDDSL | 702 |
| WP_031488318 | 634 | ERLKTYAHLFDDKVMKOLKR-RRYTGWGRLSRKLINGIRDK QSGKTILDFLK -DGf---ANRNFMQLIHDDSL | 702 |
| WP_032460140 | 634 | ERLKTYAHLFDDKVMKOLKR-RRYTGWGRLSRKLINGIRDK QSGKTILDFLK -DGf---ANRNFMQLIHDDSL | 702 |
| WP_032461047 | 634 | ERLKTYAHLEDDKVMKOLKR-RRYTGWGRLSRKLINGIRDK QSGKTILDFLK -DGf---ANRNFMQLIHDDSL | 702 |
| WP_032462016 | 634 | ERLKTYAHLFDDKVMKOLKR-RRYTGWGRLSRKLINGIRDK QSGKTILDFLK -DGf---ANRNFMQLIHDDSL | 702 |
| WP_032462936 | 634 | ERLKKYAHLFDDKVMKOLKR-RRYTGWGRLSRKLINGIRDK QSGKTILDFLK -DGf---ANRNFMQLIHDDSL | 702 |
| WP_032464890 | 634 | ERLKTYAHLEDDKVMKOLKR-RRYTGWGRLSRKLINGIRDK QSGKTILDFLK -DGf---ANRNFMQLIHDDSL | 702 |
| WP_033888930 | 459 | ERLKTYAHLEDDKVMKOLKR-RRYTGWGRLSRKLINGIRDK QSGKTILDFLK -DGf---ANRNFMQLIHDDSL | 527 |
| WP_038431314 | 634 | ERLKTYAHLEDDKVMKQLKR-RRYTGWGRLSRKLINGIRDK QSGKTILDFLK -DGf---ANRNFMQLIHDDSL | 702 |
| WP_038432938 | 634 | ERLKKYANLFDDKVMKQLKR-RHYTGWGRLSRKLINGIRDK QSGKTILDFLK -DGf---ANRNFMQLINDDSL | 702 |
| WP_038434062 | 634 | ERLKTYAHLEDDKVMKOLKR-RRYTGWGRLSRKLINGIRDK QSGKTILDFLK -DGf---ANRNFMQLIHDDSL | 702 |
| BAQ51233 | 545 | ERLKTYAHLFDDKVMKOLKR-RRYTGWGRLSRKLINGIRDK QSGKTILDFLK -DGf---ANRNFMQLIHDDSL | 613 |
| KGE60162 | | ----------------------------------------- ----------- --------------------- | |
| KGE60856 | | ----------------------------------------- ----------- --------------------- | |
| WP_002989955 | 634 | ERLKTYAHLFDDKVMKQLKR-RRYTGWGRLSRKLINGIRDK QSGKTILDFLK -DGf---ANRNFMQLIHDDSL | 702 |
| WP_003030002 | 635 | QRLOKYSDIFTKAQLKKLER-RHYTGWGRLSYKLINGIRNK ENKKTILDYLI DDG---YANRNFMQLINDDAL | 703 |
| WP_003065552 | 638 | ERLOKYSDIFTADQLKKLER-RHYTGWGRLSYKLINGIRNK ENNKTILDYLI DDG---SANRNFMQLINDDTL | 706 |
| WP_001040076 | 633 | KRLDIYKDFFTESQLKKLYR-RHYTGWGRLSAKLINGIRNK ENQKTILDYLI DDG---SANRNFMQLIKDAGL | 701 |
| WP_001040078 | 636 | KRLENYKDLFTESQLKKLYR-RHYTGWGRLSAKLINGIRDK ESQKTILDYLI DDG---RSNRNFMQLINDDGL | 704 |
| WP_001040080 | 636 | KRLENYKDLFTESQLKKLYR-RHYTGWGRLSAKLINGIRDK ESQKTILDYLI DDG---RSNRNFMQLINDDGL | 704 |
| WP_001040081 | 636 | KRLENYKDLFTESQLKKLYR-RHYTGWGRLSAKLINGIRDK ESQKTILDYLI DDG---RSNRNFMQLINDDGL | 704 |
| WP_001040083 | 636 | KRLENYKDLFTESQLKKLYR-RHYTGWGRLSAKLINGIRDK ESQKTILDYLI DDG---RSNRNFMQLINDDGL | 704 |
| WP_001040085 | 636 | KRLENYKDLFTESQLKKLYR-RHYTGWGRLSAKLINGIRDK ESQKTILDYLI DDG---RSNRNFMQLINDDGL | 704 |
| WP_001040087 | 636 | KRLENYKDLFTESQLKKLYR-RHYTGWGRLSAKLINGIRDK ESQKTILDYLI DDG---RSNRNFMQLINDDGL | 704 |
| WP_001040088 | 636 | KRLENYKDLFTESQLKKLYR-RHYTGWGRLSAKLINGIRDK ESQKTILDYLI DDG---RSNRNFMQLINDDGL | 704 |
| WP_001040089 | 636 | KRLENYKDLFTESQLKKLYR-RHYTGWGRLSAKLINGIRDK ESQKTILDYLI DDG---RSNRNFMQLINDDGL | 704 |
| WP_001040090 | 636 | KRLENYKDLFTESQLKKLYR-RHYTGWGRLSAKLINGIRDK ESQKTILDYLI DDG---RSNRNFMQLINDDGL | 704 |
| WP_001040091 | 636 | KRLENYKDLFTESQLKKLYR-RHYTGWGRLSAKLINGIRDK ESQKTILDYLI DDG---RSNRNFMQLINDDGL | 704 |
| WP_001040092 | 636 | KRLENYKDLFTESQLKKLYR-RHYTGWGRLSAKLINGIRDR ESQKTILDYLI SDG---RANRNFMQLINDDGL | 704 |
| WP_001040094 | 633 | KRLDIYKDFFTESQLKKLYR-RHYTGWGRLSAKLINGIRNK ENQKTILDYLI DDG---SANRNFMQLIKDAGL | 701 |
| WP_001040095 | 633 | KRLDIYKDFFTESQLKKLYR-RHYTGWERLSAKLINGIRNK ENQKTILDYLI DDG---SANRNFMQLIKDAGL | 701 |
| WP_001040096 | 633 | KRLDIYKDFFTESQLKKLYR-RHYTGWGRLSAKLINGIRNK ENQKTILDYLI DDG---SANRNFMQLIKDAGL | 701 |
| WP_001040097 | 633 | KRLDIYKDFFTESQLKKLYR-RHYTGWGRLSAKLINGIRNK ENQKTILDYLI DDG---SANRNFMQLIKDAGL | 701 |
| WP_001040098 | 633 | KRLDIYKDFFTESQLKKLYR-RHYTGWGRLSAKLINGIRNK ENQKTILDYLI DDG---SANRNFMQLIKDAGL | 701 |
| WP_001040099 | 633 | KRLDIYKDFFTESQLKKLYR-RHYTGWGRLSAKLINGIRNK ENQKTILDYLI DDG---SANRNFMQLIKDAGL | 701 |
| WP_001040100 | 633 | KRLDIYKDFFTESQLKKLYR-RHYTGWGRLSAKLINGIRNK ENQKTILDYLI DDG---SANRNFMQLIKDAGL | 701 |
| WP_001040104 | 636 | KRLENYKDLFTESQLKKLYR-RHYTGWGRLSAKLINGIRDK ESQKTILDYLI DDG---RSNRNFMQLINDDGL | 704 |
| WP_001040105 | 636 | KRLENYKDLFTESQLKKLYR-RHYTGWGRLSAKLINGIRDK ESQKTILDYLI DDG---RSNRNFMQLINDDGL | 704 |
| WP_001040106 | 636 | KRLENYKDLFTESQLKKLYR-RHYTGWGRLSAKLINGIRDK ESQKTILDYLI SDG---RANRNFMQLIHDDGL | 704 |
| WP_001040107 | 636 | KRLENYKDLFTESQLKKLYR-RHYTGWGRLSAKLINGIRDK ESQKTILDYLI SDG---RANRNFMQLIHDDGL | 704 |
| WP_001040108 | 636 | KRLENYKDLFTESQLKKLYR-RHYTGWGRLSAKLINGIRDK ESQKTILDYLI SDG---RANRNFMQLIHDDGL | 704 |
| WP_001040109 | 636 | KRLENYKDLFTESQLKKLYR-RHYTGWGRLSAKLINGIRDK ESQKTILDYLI SDG---RANRNFMQLIHDDGL | 704 |
| WP_001040110 | 636 | KRLENYKDLFTESQLKKLYR-RHYTGWGRLSAKLINGIRDK ESQKTILDYLI SDG---RANRNFMQLIHDDGL | 704 |
| WP_015058523 | 636 | KRLENYKDLFTESQLKKLYR-RHYTGWGRLSAKLINGIRDR ESQKTILDYLI SDG---RANRNFMQLINDDGL | 704 |
| WP_017643650 | 633 | KRLDIYKDFFTESQLKKLYR-RHYTGWGRLSAKLINGIRNK ENQKTILDYLI DDG---SANRNFMQLIKDAGL | 701 |
| WP_017647151 | 636 | KRLENYKDLFTESQLKKLYR-RHYTGWGRLSAKLINGIRDK ESQKTILDYLI DDG---KSNRNFMQLIHDDGL | 704 |
| WP_017648376 | 636 | KRLENYKDLFTESQLKKLYR-RHYTGWGRLSAKLINGIRDK ESQKTILDYLI DDG---KSNRNFMQLIHDDGL | 704 |
| WP_017649527 | 636 | KRLENYKDLFTESQLKKLYR-RHYTGWGRLSAKLINGIRDK ESQKTILDYLI DDG---RSNRNEMQLINDDGL | 704 |
| WP_017771611 | 636 | KRLENYKDLFTESQLKKLYR-RHYTGWGRLSAKLINGIRDK ESQKTILDYLI SDG---RANRNFMOLIHDDGL | 704 |
| WP_017771984 | 636 | KRLENYKDLFTESQLKKLYR-RHYTGWGRLSAKLINGIRDK ESQKTILDYLI DDG---RSNRNFMQLINDDGL | 704 |
| CFQ25032 | 636 | KRLENYKDLFTESQLKKLYR-RHYTGWGRLSAKLINGIRDK ESQKTILDYLI DDG---RSNRNFMQLINDDGL | 704 |
| CFV16040 | 636 | KRLENYKDLFTESQLKKLYR-RHYTGWGRLSAKLINGIRDK ESQKTILDYLI DDG---RSNRNFMQLINDDGL | 704 |
| KLJ37842 | 636 | KRLENYKDLFTESQLKKLYR-RHYTGWGRLSAKLINGIRDK ESQKTILDYLI DDG---RSNRNFMQLINDDGL | 704 |
| KLJ72361 | 636 | KRLENYKDLFTESQLKKLYR-RHYTGWGRLSAKLINGIRDK ESQKTILDYLI DDG---RSNRNFMQLINDDGL | 704 |
| KLL20707 | 636 | KRLENYKDLFTESQLKKLYR-RHYTGWGRLSAKLINGIRDK ESQKTILDYLI DDG---RSNRNFMQLINDDGL | 718 |
| KLL42645 | 636 | KRLENYKDLFTESQLKKLYR-RHYTGWGRLSAKLINGIRDK ESQKTILDYLI SDG---RANRNFMQLIHDDGL | 704 |
| WP_047207273 | 636 | KRLENYKDLFTESQLKKLYR-RHYTGWGRLSAKLINGIRDK ESQKTILDYLI DDG---RSNRNFMQLINDDGL | 704 |
| WP_047209694 | 633 | KRLDIYKDFFTESQLKKLYR-RHYTGWGRLSAKLINGIRNK ENQKTILDYLI DDG---SANRNFMQLIKDAGL | 701 |
| WP_050198062 | 636 | KRLENYKDLFTESQLKKLYR-RHYTGWGRLSAKLINGIRDK ESQKTILDYLI DDG---RSNRNFMQLINDDGL | 704 |
| WP_050201642 | 636 | KRLENYKDLFTESQLKKLYR-RHYTGWGRLSAKLINGIRDK ESQKTILDYLI DDG---RSNRNFMQLINDDGL | 704 |
| WP_050204027 | 636 | KRLENYKDLFTESQLKKLYR-RHYTGWGRLSAKLINGIRDK ESQKTILDYLI SDG---RANRNFMQLIHDDGL | 704 |
| WP_050881965 | 636 | KRLENYKDLFTESQLKKLYR-RHYTGWGRLSAKLINGIRDK ESQKTILDYLI DDG---RSNRNFMQLINDDGL | 704 |
| WP_050886065 | 636 | KRLENYKDLFTESQLKKLYR-RHYTGWGRLSAKLINGIRDK ESQKTILDYLI DDG---RSNRNFMQLINDDGL | 704 |
| AHN30376 | 636 | KRLENYKDLFTESQLKKLYR-RHYTGWGRLSAKLINGIRDR ESQKIILDYLI SDG---RANRNFMQLINDDGL | 704 |
| EAO78426 | 636 | KRLENYKDLFTESQLKKLYR-RHYTGWGRLSAKLINGIRDK ESQKTILDYLI DDG---RSNRNFMQLINDDGL | 704 |
| CCW42055 | 636 | KRLDIYKDFFTESQLKKLYR-RHYTGWGRLSAKLINGIRNK ENQKTILDYLI DDG---SANRNFMQLIKDAGL | 704 |
| WP_003041502 | 635 | QRLQKYSDIFTKAQLKKLER-RHYTGWGRLSYKLINGIRNK ENKKTILDYLI DDG---YANRNFMQLINDDAL | 703 |
| WP_037593752 | 636 | QRLQKYSDIFTKAQLKKLER-RHYTGWGRLSYKLINGIRNK ENKKTILDYLI DDG---YANRNFMQLINDDAL | 704 |
| WP_049516684 | 636 | QRLOKYSDIFTTQQLKKLER-RHYTGWGRLSYKLINGIRNK ENKKTILDYLI DDG---YANRNFMQLINDDAL | 704 |
| GAD46167 | 635 | QRLOKYSDIFTKAQLKKLER-RHYTGWGRLSYKLINGIRNK ENKKTILDYLI DDG---YANRNFMQLINDDAL | 703 |
| WP_018363470 | 636 | QRLOKYSDIFTKQQLKKLER-RHYTGWGRLSYKLINGIRNK ENNKTILDYLI DDG---SANRNFMQLINDDAL | 704 |
| WP_003043819 | 644 | ERLKTYAHLFDDKVMKOLKR-RHYTGWGRLSRKMINGIRDK QSGKTILDELK -DGf---SNRNFMQLIHDDSL | 712 |
| WP_006269658 | 635 | QRLOKYSDIFTKAQLKKLER-RHYTGWGRLSYKLINGIRNK ENKKTILDYLI DDG---YANRNFMQLINDDAL | 703 |
| WP_048800889 | 635 | QRLQKYSDIFTKAQLKKLER-RHYTGWGRLSYKLINGIRNK ENNKTILEYLV DDG---YANRNFMQLINDDTL | 703 |
| WP_012767106 | 634 | ERLKKYANLFDDKVMKOLKR-RHYTGWGRLSRKLINGIRDK QSGKTILDFLK -DGf---ANRNFMQLINDDSL | 702 |
| WP_014612333 | 634 | ERLKKYANLEDDKVMKOLKR-RHYTGWGRLSRKLINGIRDK QSGKTILDFLK -DGf---ANRNFMQLINDDSL | 702 |
| WP_015017095 | 634 | ERLKTYAHLEDDKVMKOLKR-RHYTGWGRLSRKLINGIRDK QSGKTILDFLK -DGf---ANRNFIQLIHDDSL | 702 |
| WP_015057649 | 634 | ERLKKYANLFDDKVMKQLKR-RHYTGWGRLSRKLINGIRDK QSGKTILDFLK -DGf---ANRNFMQLINDDSL | 702 |
| WP_048327215 | 634 | ERLKTYAHLFDDKVMKOLKR-RHYTGWGRLSRKLINGIRDK QSGKTILDFLK -DGf---ANRNFIQLIHDDSL | 702 |
| WP_049519324 | 634 | ERLKTYAHLEDDKVMKOLKR-RHYTGWGRLSRKLINGIRDK QSGKTILDFLK -DGf---ANRNFIQLIHDDSL | 702 |
| WP_012515931 | 634 | KRLDQYAHLFDKVVINKLER-HHYTGWGRLSGKLINGIRDK QSGKTILDFLK -DGf---ANRNFMQLIHDSEL | 702 |
| WP_021320964 | 634 | KRLDQYAHLFDKVVINKLER-HHYTGWGRLSGKLINGIRDK QSGKTILDFLK -DGf---ANRNFMQLIHDSEL | 702 |
| WP_037581760 | 634 | KRLDQYAHLFDKVVLNKLER-HHYTGWGRLSGKLINGIRDK QSGKTILDFLK -DGf---ANRNFMQLIHDSEL | 702 |
| WP_004232481 | 635 | ERLOKYSDIFTSQQLKKLER-RHYTGWGRLSYKLINGIRNK ENNKTILDFLI DDG---DANRNFMQLINDDSL | 703 |
| WP_009854540 | 636 | ERLOKYSDIFTANQLKKLER-RHYTGWGRLSYKLINGIRNK ENNKTILDYLI DDG---SANRNFMQLINDDTL | 704 |
| WP_012962174 | 636 | QRLQKYSDIFTPQQLKKLER-RHYTGWGRLSYKLINGIRNK ENGKSILDYLI DDG---YANRNFMQLISDDTL | 704 |
| WP_039695303 | 638 | ERLOKYSDIFTANQLKKLER-RHYTGWGRLSYKLINGIRNK ENNKTILDYLI DDG---SANRNFMQLINDDTL | 706 |
| WP_014334983 | 635 | ERLQKYSDFFTSQQLKKLER-RHYTGWGRLSYKLINGIRNK ENNKTILDFLI DDG---HANRNEMQLINDESL | 703 |
| WP_003099269 | 634 | RRLVKYADVFEKSVLKKLKK-RHYTGWGRLSQKLINGIKDK QTGKTILGFLK -DGv---ANRNFMQLINDSSL | 702 |
| AHY15608 | 634 | RRLVKYADVFEKSVLKKLKK-RHYTGWGRLSQKLINGIKDK QTGKTILGFLK -DGv---ANRNFMQLINDSSL | 702 |
| AHY17476 | 634 | RRLVKYADVFEKSVLKKLKK-RHYTGWGRLSQKLINGIKDK QTGKTILGFLK -DGv---ANRNFMQLINDSSL | 702 |
| ESR09100 | | ----------------------------------------- ----------- --------------------- | |
| AGM98575 | 634 | RRLVKYADVFEKSVLKKLKK-RHYTGWGRLSQKLINGIKDK QTGKTILGFLK -DGv---ANRNFMQLINDSSL | 702 |
| ALF27331 | 635 | KRLENYSDLLTKEQVKNLER-RHYTGWGRLSAELIHGIRNK ESRKTILDYLI DDG---NSNRNFMQLINDDAL | 703 |
| WP_018372492 | 648 | KRLSELNIPFENKIIKKLAR-KKYTGWGNLSRKLIDGIRNR ETNRTILGHLI DDGf---SNRNLMQLINDDGL | 716 |
| WP_045618028 | 634 | QRLAHYASIFDEKVIKALTR-RHYTGWGKLSAKLINGIYDK QSKKTILDYLI DDG---EINRNFMQLINDDGL | 702 |
| WP_045635197 | 633 | QRLAQYDSLFDEKVIKALTR-RHYTGWGKLSAKLINGICDK QTGNTILDYLI DDG---KINRNFMQLINDDGL | 701 |
| WP_002263549 | 635 | KRLENYSDLLTKEQVKKLER-RHYTGWGRLSAELIHGIRNK ESRKTILDYLI DDG---NSNRNFMQLINDDAL | 703 |
| WP_002263887 | 635 | KRLENYSDLLTKEQVKKLER-RHYTGWGRLSAELIHGIRNK ESRKTILDYLI DDG---NSNRNFMQLINDDAL | 703 |
| WP_002264920 | 635 | KRLKNYSDLLTKEQLKKLER-RHYTGWGRLSAELIHGIRNK ESRKTILDYLI DDG---NSNRNFMQLINDDAL | 703 |
| WP_002269043 | 635 | KRLKNYSDLLTKEQLKKLER-RHYTGWGRLSAELIHGIRNK ESRKTILDYLI DDG---NSNRNFMQLINDDAL | 703 |
| WP_002269448 | 635 | KRLENYSDLLTKEQVKKLER-RHYTGWGRLSAELIHGIRNK ESRKTILDYLI DDG---NSNRNFMQLINDDAL | 703 |
| WP_002271977 | 635 | KRLENYSDLLTKEQVKKLER-RHYTGWGRLSAELIHGIRNK ESRKTILDYLI DDG---NSNRNFMQLINDDAL | 703 |
| WP_002272766 | 635 | KRLENYSDLLTKEQVKKLER-RHYTGWGRLSAELIHGIRNK ESRKTILDYLI DDG---NSNRNFMQLINDDAL | 703 |
| WP_002273241 | 635 | KRLENYSDLLTKEQVKKLER-RHYTGWGRLSAELIHGIRNK ESRKTILDYLI DDG---NSNRNFMQLINDDAL | 703 |
| WP_002275430 | 635 | KRLENYSDLLTKEQVKKLER-RHYTGWGRLSAELIHGIRNK ESRKTLLDYLI DDG---NSNRNFMQLINDDAL | 703 |
| WP_002276448 | 635 | KRLENYSDLLTKEQVKKLER-RHYTGWGRLSAELIHGIRNK ESRKTILDYLI DDG---NSNRNFMQLINDDAL | 703 |
| WP_002277050 | 636 | QRLAKYADVEDKKVIDQLAR-RHYTGWGRLSAKLLNGIRDK QSCKTIMDYLI DDA---QSNRNLMQLITDDNL | 704 |
| WP_002277364 | 635 | KRLENYSDLLTKEQVKKLER-RHYTGWGRLSAELIHGIRNK ESRKTILDYLI DDG---NSNRNFMQLINDDAL | 703 |
| WP_002279025 | 635 | KRLKNYSDLLTKEQLKKLER-RHYTGWGRLSAELIHGIRNK ESRKTILDYLI DDG---NSNRNFMQLINDDAL | 703 |
| WP_002279859 | 635 | KRLKNYSDLLTKEQLKKLER-RHYTGWGRLSAELIHGIRNK ESRKTILDYLI DDG---NSNRNFMQLINDDAL | 703 |
| WP_002280230 | 635 | KRLENYSDLLTKEQVKKLER-RHYTGWGRLSAELIHGIRNK ESRKTILDYLI DDG---NSNRNFMQLINDDAL | 703 |
| WP_002281696 | 635 | KRLENYSDLLTKEQVKKLER-RHYTGWGRLSAELIHGIRNK ESRKTILDYLI DDG---NSNRNFMQLINDDAL | 703 |
| WP_002282247 | 636 | QRLAKYADVEDKKVIDQLAR-RHYTGWGRLSAKLLNGIRDK QSCKTIMDYLI DDA---QSNRNLMQLITDDNL | 704 |
| WP_002282906 | 635 | KRLENYSDLLTKEQVKKLER-RHYTGWGRLSAELIHGIRNK ESRKTILDYLI DDG---NSNRNFMQLINDDAL | 703 |
| WP_002283846 | 635 | KRLKNYSDLLTKEQLKKLER-RHYTGWGRLSAELIHGIRNK ESRKTILDYLI DDG---NSNRNFMQLINDDAL | 703 |
| WP_002287255 | 635 | KRLENYSDLLIKEQVKKLER-RHYTGWGRLSAELIHGIRNK ESRKTILDYLI DDG---NSNRNFMQLINDDAL | 703 |
| WP_002288990 | 635 | KRLKNYSDLLTKEQLKKLER-RHYTGWGRLSAELIHGIRNK ESRKTILDYLI DDG---NSNRNFMQLINDDAL | 703 |
| WP_002289641 | 635 | KRLENYSDLLTKEQVKKLER-RHYTGWGRLSAELIHGIRNK ESRKTILDYLI DDG---NSNRNFMQLINDDAL | 703 |
| WP_002290427 | 635 | KRLKNYSDLLTKEQLKKLER-RHYTGWGRLSAELIHGIRNK ESRKTILDYLI DDG---NSNRNFMQLINDDAL | 703 |
| WP_002295753 | 635 | KRLENYSDLLTKEQVKKLER-RHYTGWGRLSAELIHGIRNK ESRKTILDYLI DDG---NSNRNFMQLINDDAL | 703 |
| WP_002296423 | 635 | KRLKNYSDLLTKEQLKKLER-RHYTGWGRLSAELIHGIRNK ESRKTILDYLI DDG---NSNRNFMQLINDDAL | 703 |
| WP_002304487 | 645 | QRLQKYSDIFTKAQLKKLER-RHYTGWGRLSYKLINGIRDK QSNKTILGYLI DDG---YSNRNFMQLINDDAL | 713 |
| WP_002305844 | 635 | KRLENYSDLLIKEQVKKLER-RHYTGWGRLSAELIHGIRNK ESRKTILDYLI DDG---NSNRNFMQLINDDAL | 703 |
| WP_002307203 | 635 | KRLENYSDLLTKEQVKKLER-RHYTGWGRLSAELIHGIRNK ESRKTLLDYLI DDG---NSNRNFMQLINDDAL | 703 |
| WP_002310390 | 635 | KRLKNYSDLLTKEQLKKLER-RHYTGWGRLSAELIHGIRNK ESRKTILDYLI DDG---NSNRNFMQLINDDAL | 703 |
| WP_002352408 | 635 | KRLENYSDLLTKEQVKKLER-RHYTGWGRLSAELIHGIRNK ESRKTLLDYLI DDG---NSNRNFMQLINDDAL | 703 |
| WP_012997688 | 635 | KRLENYSDLLTKEQVKKLER-RHYTGWGRLSAELIHGIRNK ESRKTILDYLI DDG---NSNRNFMQLINDDAL | 703 |
| WP_014677909 | 635 | KRLKNYSDLLTKEQLKKLER-RHYTGWGRLSAELIHGIRNK ESRKTILDYLI DDG---NSNRNFMQLINDDAL | 703 |
| WP_019312892 | 635 | KRLENYSDLLTKEQVKKLER-RHYTGWGRLSAELIHGIRNK ESRKTILDYLI DDG---NSNRNFMQLINDDAL | 703 |
| WP_019313659 | 635 | KRLENYSDLLTKEQVKKLER-RHYTGWGRLSAELIHGIRNK ESRKTILDYLI DDG---NSNRNFMQLINDDAL | 703 |
| WP_019314093 | 635 | KRLENYSDLLTKEQVKKLER-RHYTGWGRLSAELIHGIRNK ESRKTLLDYLI DDG---NSNRNFMQLINDDAL | 703 |
| WP_019315370 | 635 | KRLENYSDLLTKEQVKKLER-RHYTGWGRLSAELIHGIRNK ESRKTLLDYLI DDG---NSNRNFMQLINDDAL | 703 |
| WP_019803776 | 635 | KRLENYSDLLTKEQVKKLER-RHYTGWGRLSAELIHGIRNK ESRKTILDYLI DDG---NSNRNFMQLINDDAL | 703 |
| WP_019805234 | 635 | KRLKNYSDLLTKEQLKKLER-RHYTGWGRLSAELIHGIRNK ESRKTILDYLI DDG---NSNRNFMQLINDDAL | 703 |
| WP_024783594 | 635 | KRLENYSDLLTKEQVKKLER-RHYTGWGRLSAELIHGIRNK ESRKTILDYLI DDG---NSNRNFMQLINDDAL | 703 |
| WP_024784288 | 636 | QRLAKYADVEDKKVIDQLAR-RHYTGWGRLSAKLLNGIRDK QSCKTIMDYLI DDA---QSNRNLMQLITDDNL | 704 |
| WP_024784666 | 635 | KRLENYSDLLTKEQVKKLER-RHYTGWGRLSAELIHGIRNK ESRKTILDYLI DDG---NSNRNFMQLINDDAL | 703 |
| WP_024784894 | 635 | KRLENYSDLLTKEQVKKLER-RHYTGWGRLSAELIHGIRNK ESRKTILDYLI DDG---NSNRNFMQLINDDAL | 703 |
| WP_024786433 | 636 | QRLAKYADVFDKKVIDQLAR-RHYTGWGRLSAKLLNGIRDK QSCKTIMDYLI DDA---QSNRNLMQLITDDNL | 704 |
| WP_049473442 | 635 | KRLENYSDLLTKEQVKKLER-RHYTGWGRLSAELIHGIRNK ESRKTILDYLI DDG---NSNRNFMQLINDDAL | 703 |
| WP_049474547 | 635 | KRLENYSDLLTKEQVKKLER-RHYTGWGRLSAELIHGIRNK ESRKTILDYLI DDG---NSNRNFMQLINDDAL | 703 |
| EMC03581 | 628 | KRLENYSDLLTKEQVKKLER-RHYTGWGRLSAELIHGIRNK ESRKTILDYLI DDG---NSNRNFMQLINDDAL | 696 |
| WP_000428612 | 636 | QRLAQYDSLFDEKVIKALTR-RHYTGWGKLSSKLINGIRDK QTGKTILDYLM DDG---YNNRNFMQLINDDEL | 704 |
| WP_000428613 | 634 | QRLAQYDSLFDEKVIKALIR-RHYTGWGKLSAKLIDGICDK QTGNTILDYLI DDG---KNNRNFMQLINDDGL | 702 |
| WP_049523028 | 633 | QRLNQYDSIFDEKVIKALTR-RHYTGWGKLSAKLINGIRDK KTSKTILDYLI DDG---YSNRNFMQLINDDGL | 701 |
| WP_003107102 | 603 | KRLSKYESIFDPSILKKLKK-RHYTGWGRLSQKLINGIRDK QTGKTILDFLI -DGq---ANRNFMQLINDPSL | 671 |
| WP_054279288 | 636 | NRLAVYEDLFDQNVLKOLKR-RHYTGWGRLSKOLINGMRDK HTGKTILDFLK -DGf---INRNFMQLINDDNL | 704 |
| WP_049531101 | 634 | QRLAQYASIFDEKVIKTLTR-RHYTGWGKLSAKLINCIRDR KTGKTILDYLI DDG---YNNRNFMQLINDDGL | 702 |
| WP_049538452 | 634 | QRLAQYDSIFDEKVIKALTR-RHYTGWGKLSAKLINGIRDK QTGKTILDYLI DDG---YSNRNEMQLINDDGL | 702 |
| WP_049549711 | 634 | QRLAQYDSLFDKKVIKALTR-RHYTGWGKLSAKLINGICDK QTGNTILDYLI DDG---EINRNFMQLINDDGL | 702 |
| WP_007896501 | 637 | KRLAKYANLFEKSVLKKLRK-RHYRGWGRLSRQLIDGMKDK ASGKTILDFLK -DDf---ANRNFIQLINDSSL | 705 |
| EFR44625 | 589 | KRLAKYANLFEKSVLKKLRK-RHYRGWGRLSRQLIDGMKDK ASGKTILDFLK -DDf---ANRNFIQLINDSSL | 657 |
| WP_002897477 | 633 | QRLAQYDTLFDEKVIKALTR-RHYTGWGKLSAKLINGIRDK QSGKTILDYLI DDD---KINRNFMQLINDDGL | 701 |
| WP_002906454 | 633 | QRLAQYDTLFDEKVIKALTR-RHYTGWGKLSAKLINGIRDK QTGKTILEYLI DDG---DCNRNFMQLINDDGL | 701 |
| WP_009729476 | 634 | QRLAQYDSLFDEKVIKALTR-RHYTGWGKLSAKLINGISDK QTGNTILDYLI DDG---EINRNFMQLINDDGL | 702 |
| COR24647 | 637 | QRLLKYEDIFSKKVIANLTR-RHYTGWGRLSAKLINGIKDK HSRKTILDYLI DDG---HSNRNFMQLINDDNL | 705 |
| WP_000066813 | 636 | QRLAQYDSLFDEKVIKALTR-RHYTGWGKLSAKLINGIRDK KSGKTILDYLI DDG---EINRNFMQLIHDDGL | 704 |
| WP_009754323 | 634 | QRLAQYDSIFDEKVIKALTR-RHYTGWGKLSAKLINGICDK KTGKTILDYLI DDG---YNNRNFMQLINDDGL | 702 |
| WP_044674937 | 633 | KRLEKYKDILTEEQRKKLER-RHYTGWGRLSAKLINGILDK VTRKTILGYLI DDG---TSNRNFMQLINDDTL | 701 |
| WP_044676715 | 635 | KRLEKYKDVLTEEQRKKLER-RHYTGWGRLSAKLINGIRDK VTRKTILDYLI DDG---TSNRNFMQLINDDTL | 703 |
| WP_044680361 | 635 | KRLEKYKDVLTEEQRKKLER-RHYTGWGRLSAKLINGIRDK VTRKTILDYLI DDG---TSNRNFMQLINDDTL | 703 |
| WP_044681799 | 633 | KRLEKYKDILTEEQRKKLER-RHYTGWGRLSAKLINGILDK VTRKTILGYLI DDG---TSNRNFMQLINDDTL | 701 |
| WP_049533112 | 635 | QRLOKYSDIFTKAQLKKLER-CHYTGWGRLSYKLINGIRNK ENKKTILDYLI DDG---YANRNFMQLINDDAL | 703 |
| WP_029090905 | 613 | RKLSEYPQLTEQQQVOLAQV--RFRGWGRLSQRLINRIKTP EDHKLSINEIL ------QTNENFMQIIRNKDY | 682 |
| WP_006506696 | 638 | RRLKKKYALPDDKVKQILKL--KYKDWSRLSKKLLDGIVAD SV--TVLDVLE -------SRLNLMEIINDKDL | 705 |
| AIT42264 | 634 | ERLKTYAHLFDDKVMKOLKR-RRYTGWGRLSRKLINGIRDK QSGKTILDFLK -DGf---ANRNFMQLIHDDSL | 702 |
| WP_034440723 | 638 | RQLMKFKDKLSEKAINQLSK-KHYTGWGQLSEKLINGIRDE QSNKTILDYLI DNGcpkNMNRNFMQLINDDTL | 710 |
| AKQ21048 | 634 | ERLKTYAHLEDDKVMKOLKR-RRYTGWGRLSRKLINGIRDK QSGKTILDFLK -DGf---ANRNFMQLIHDDSL | 702 |
| WP_004636532 | 634 | KQLQTYSDTLSPEILKKLER-KHYTGWGRFSKKLINGLRDE GSNKTILDYLK DEGssgPTNRNFMQLIRDNTL | 706 |
| WP_002364836 | 642 | TQLSTFKGQFSAEVLKKLER-KHYTGWGRLSKKLINGIYDK ESGKTILGYLI DDGvskHYNRNFMQLINDSQL | 714 |
| WP_016631044 | 593 | TQLSTFKGQFSAEVLKKLER-KHYTGWGRLSKKLINGIYDK ESGKTILDYLV DDGvskHYNRNFMQLINDSQL | 665 |
| EMS75795 | 382 | TQLKKYQSVLGDGFFKKLVK-KHYTGWGRLSERLINGIRDK KTNKTILDYLI DDDfpyNRNRNFMQLINDDSL | 454 |
| WP_002373311 | 642 | TOLSTFKGQFSAEVLKKLER-KHYTGWGRLSKKLINGIYDK ESGKTILDYLV DDGvskHYNRNFMQLINDSQL | 714 |
| WP_002378009 | 642 | TOLSTFKGQFSAEVLKKLER-KHYTGWGRLSKKLINGIYDK ESGKTILDYLI DDGvskHYNRNFMQLINDSQL | 714 |
| WP_002407324 | 642 | TOLSTFKGQFSAEVLKKLER-KHYTGWGRLSKKLINGIYDK ESGKTILDYLV DDGvskHYNRNFMQLINDSQL | 714 |
| WP_002413717 | 642 | TOLSTFKGQFSAEVLKKLER-KHYTGWGRLSKKLINGIYDK ESGKTILDYLV DDGvSkHYNRNFMQLINDSQL | 714 |
| WP_010775580 | 644 | TOLSTFKGOFSEEVLKKLER-KHYTGWGRLSKKLINGIYDK ESGKTILDYLI DDGVSKHYNRNFMQLINDSQL | 716 |
| WP_010818269 | 642 | TOLSTFKGOFSAEVLKKLER-KHYTGWGRLSKKLINGIYDK ESGKTILGYLI DDGvskHYNRNFMQLINDSQL | 714 |
| WP_010824395 | 642 | TOLSTFKGOFSAEVLKKLER-KHYTGWGRLSKKLINGIYDK ESGKTILDYLI DDGvskHYNRNFMQLINDSQL | 714 |
| WP_016622645 | 642 | TOLSTFKGQFSAEVLKKLER-KHYTGWGRLSKKLINGIYDK ESGKTILDYLV DDGvSkHYNRNFMQLINDSQL | 714 |
| WP_033624816 | 642 | TQLSTFKGQFSAEVLKKLER-KHYTGWGRLSKKLINGIYDK ESGKTILDYLI DDGvskHYNRNFMQLINDSQL | 714 |
| WP_033625576 | 642 | TQLSTFKGOFSAEVLKKLER-KHYTGWGRLSKKLINGIYDK ESGKTILDYLI DDGvskHYNRNFMQLINDSQL | 714 |
| WP_033789179 | 642 | TOLSTFKGQFSEEVLKKLER-KHYTGWGRLSKKLINGIYDK ESGKTILDYLI DDGvskHYNRNFMQLINDSQL | 714 |
| WP_002310644 | 642 | TRLSHHEATLGKHIIKKLTK-KHYTGWGRLSKELIQGIRDK QSNKTILDYLI DDDfphHRNRNFMQLINDDSL | 714 |
| WP_002312694 | 643 | TRLSHHEATLGKHIIKKLTK-KHYTGWGRLSKELIQGIRDK QSNKTILDYLI DDDfphHRNRNFMQLINDDSL | 715 |
| WP_002314015 | 643 | TRLSHHEATLGKHIIKKLTK-KHYTGWGRLSKELIQGIRDK QSNKTILDYLI DDDfphHRNRNFMQLINDDSL | 715 |
| WP_002320716 | 643 | TRLSHHEATLGKHIIKKLTK-KHYTGWGRLSKELIQGIRDK QSNKTILDYLI DDDfphHRNRNFMQLINDDSL | 715 |
| WP_002330729 | 642 | TRLSHHEATLGKHIIKKLTK-KHYTGWGRLSKELIQGIRDK QSNKTILDYLI DDDfphHRNRNFMQLINDDSL | 714 |
| WP_002335161 | 643 | TRLSHHEATLGKHIIKKLTK-KHYTGWGRLSKELIQGIRDK QSNKTILDYLI DDDfphHRNRNFMQLINDDSL | 715 |
| WP_002345439 | 643 | TRLSHHEATLGKHIIKKLTK-KHYTGWGRLSKELIQGIRDK QSNKTILDYLI DDDfphHRNRNCMQLINDDSL | 715 |
| WP_034867970 | 638 | HQLSKYQEVFGEKLLKEFAR-KHYTGWGRFSAKLIHGIRDR KINKTILDYLI DDDvpaNRNRNLMQLINDEHL | 710 |
| WP_047937432 | 643 | TRLSHHEATLGKHIIKKLTK-KHYTGWGRLSKELIQGIRDK QSNKTILDYLI DDDfphHRNRNFMQLINDDSL | 715 |
| WP_010720994 | 638 | HQLSKYQEVFGEKLLKEFAR-KHYTGWGRFSAKLIHGIRDR KINKTILDYLI DDDvpaNRNRNLMQLINDEHL | 710 |
| WP_010737004 | 638 | HQLSKYQEVFGEKLLKEFAR-KHYTGWGRFSAKLIHGIRDR KINKTILDYLI DDDvpaNRNRNLMQLINDEHL | 710 |
| WP_034700478 | 638 | HQLSKYQEVFGEKLLKEFAR-KHYTGWGRFSAKLIHGIRDR KTNKTILDYLI DDDvpaNRNRNLMQLINDEHL | 710 |
| WP_007209003 | 635 | NQLEQLPLNLSTKTIKALSR-RKYTGWGRLSARLIDGIHDK NSGKTILDYLI DESdsyIVNRNFMQLINDDHL | 707 |
| WP_023519017 | 632 | EQLKPYETVLGLPAIKKLAK-KHYTGWGRLSEKMIQGMREK QSRKTILDYLI DDDfpcNRNRNFMQLINDDHL | 704 |
| WP_010770040 | 635 | EQLKKYTYLFDEEVLKKLER-RHYTGWGRLSAKLLIGIKEK RTHKTILDYLI DDGgkqPINRNLMQLINDSDL | 707 |
| WP_048604708 | 631 | EQLSKFSDRLSEKTIKDLER-RHYTGWGRLSAKLINGIHDK QSNKTILDYLI DDApkkNINRNFMQLINDNRL | 703 |
| WP_010750235 | 637 | TQLKKYQRILGEEIFKKLVK-KKYTGWGRLSKRLINGIRDQ KINKTILDYLI DDDfpyNRNRNFMQLINDDHL | 709 |
| AII16583 | 673 | ERLKTYAHLFDDKVMKOLKR-RRYTGWGRLSRKLINGIRDK QSGKTILDFLK -DGf---ANRNFMQLIHDDSL | 741 |
| WP_029073316 | 648 | RRLKKEYDLDEEKIKKILKL--KYSGWSRLSKKLLSGIKTK RTPETVLEVME -------TNMNLMQVINDEKL | 717 |
| WP_031589969 | 648 | RRLKKEYDLDEEKIKKILKL--KYSGWSRLSKKLLSGIKTK RTPETVLEVME -------TNMNLMQVINDEKL | 717 |
| KDA45870 | 631 | RRLENYRDFLGEDILRKLSR-KKYTGWGRLSAKLLDGIYDK KTHKTILDCLM EDYS-----QNFMQLINDDTY | 698 |
| WP_039099354 | 651 | AKLNEIDWLTDQQRVOLAAK--RYRGWGRLSAKLLTQIVN- ANGQRIMDLLW -------TTDNFMRIVHSE-- | 712 |
| AKP02966 | 633 | EKLHSSNYSYTSDQIKKISN-MRYKGWGRLSKKILTCITTE TNTPKSLQLSN -DLm-wTTNNNFISIISNDKY | 706 |
| WP_010991369 | 638 | EQLQQFSDVLDGVVLKKLER-RHYTGWGRLSAKLLMGIRDK QSHLTILDYLM DDG----LNRNLMQLINDSNL | 706 |
| WP_033838504 | 638 | EQLQQFSDVLDGVVLKKLER-RHYTGWGRLSAKLLMGIRDK QSHLTILDYLM DDG----LNRNLMQLINDSNL | 706 |
| EHN60060 | 641 | EQLQQFSDVLDGVVLKKLER-RHYTGWGRLSAKLLMGIRDK QSHLTILDYLM DDG----LNRNLMQLINDSNL | 709 |
| EFR89594 | 407 | EQLQQFSDVLDGVVLKKLER-RHYTGWGRLSAKLLMGIRDK QSHLTILDYLM DDG----LNRNLMQLINDSNL | 475 |
| WP_038409211 | 638 | EQLQSFSDVLDGTILKKLER-RHYTGWGRLSAKLLTGIRDK HSHLTILDYLM DDG----LNRNLMQLINDSNL | 706 |
| EFR95520 | 257 | EQLQSFSDVLDGTILKKLER-RHYTGWGRLSAKLLTGIRDK HSHLTILDYLM DDG----LNRNLMQLINDSNL | 325 |
| WP_003723650 | 638 | EQLQQFSDVLDGGVLKKLER-RHYTGWGRLSAKLLVGIREK QSHLTILDYLM DDG----LNRNLMQLINDSNL | 706 |
| WP_003727705 | 638 | EQLEQFSDVLDGVVLKKLER-RHYTGWGRLSAKLLVGIRDK QSHLTILDYLM DDG----LNRNLMQLINDSNL | 706 |
| WP_003730785 | 638 | EQLEQFSDVLDGVVLKKLER-RHYTGWGRLSAKLLVGIRDK QSHLTILDYLM DDG----LNRNLMQLINDSNL | 706 |
| WP_003733029 | 638 | EQLQQFSDVLDGTVLKKLER-RHYTGWGRLSAKLLVGIRDK QSHLTILDYLM DDG----LNRNLMQLINDSNL | 706 |
| WP_003739838 | 638 | EQLQQFSDVLDGAVLKKLER-RHYTGWGRLSAKLLVGIRDK QSHLTILDYLM DDG----LNRNLMQLINDSNL | 706 |
| WP_014601172 | 638 | EQLQQFSDVLDGGVLKKLER-RHYTGWGRLSAKLLVGIREK QSHLTILDYLM DDG----LNRNLMQLINDSNL | 706 |
| WP_023548323 | 638 | EQLQQFSDVLDGTVLKKLER-RHYTGWGRLSAKLLVGIRDK QSHLTILDYLM DDG----LNRNLMQLINDSNL | 706 |
| WP_031665337 | 638 | EQLQQFSDVLDGVVLKKLER-RHYTGWGRLSAKLLVGIRDK QSHLTILEYLM DDG----LNRNLMQLINDSNL | 706 |
| WP_031669209 | 638 | EQLQQFSDVLDGTVLKKLER-RHYTGWGRLSAKLLVGIRDK QSHLTILDYLM DDG----LNRNLMQLINDSNL | 706 |
| WP_033920898 | 638 | EQLQQFSDVLDGTVLKKLER-RHYTGWGRLSAKLLVGIRDK QSHLTILDYLM DDG----LNRNLMQLINDSNL | 706 |
| AKI42028 | 641 | EQLQQFSDVLDGGVLKKLER-RHYTGWGRLSAKLLVGIREK QSHLTILDYLM DDG----LNRNLMQLINDSNL | 709 |
| AKI50529 | 641 | EQLQQFSDVLDGTVLKKLER-RHYTGWGRLSAKLLVGIRDK QSHLTILDYLM DDG----LNRNLMQLINDSNL | 709 |
| EFR83390 | 86 | EQLQQFSDVLDGVVLKKLER-RHYTGWGRLSAKLLVGIRDK QSHLTILEYLM DDG----LNRNLMQLINDSNL | 154 |
| WP_046323366 | 638 | ERLQEFSNVLDEAVLKKLER-RHYTGWGRLSAKLLIGIRDK ESHLTILDYLM DDK----HNRNLMQLINDSNL | 706 |
| AKE81011 | 650 | ERLKTYAHLFDDKVMKOLKR-RRYTGWGRLSRKLINGIRDK QSGKTILDFLK -DGf---ANRNFMOLIHDDSL | 718 |
| CUO82355 | 642 | RRLKKKYALPDDKIKQILKL--KYKDWSRLSKKLLDGIVAD SV--TVLDVLE -------SRLNLMEIINDKEL | 709 |
| WP_033162887 | 645 | RRLKKVYQLDDLLVDKILKL--NYTGWSRLSEKLLTGMTAD KA--TVLFVLE -------SNKNLMEIINDEKI | 712 |
| AGZ01981 | 667 | ERLKTYAHLFDDKVMKOLKR-RRYTGWGRLSRKLINGIRDK QSGKTILDFLK -DGf---ANRNFMQLIHDDSL | 735 |
| AKA60242 | 634 | ERLKTYAHLFDDKVMKOLKR-RRYTGWGRLSRKLINGIRDK QSGKTILDFLK -DGf---ANRNFMQLIHDDSL | 702 |
| AKS40380 | 634 | ERLKTYAHLEDDKVMKOLKR-RRYTGWGRLSRKLINGIRDK QSGKTILDFLK -DGf---ANRNFMQLIHDDSL | 702 |
| 4UN5_B | 638 | ERLKTYAHLEDDKVMKOLKR-RRYTGWGRLSRKLINGIRDK QSGKTILDFLK -DGf---ANRNFMQLIHDDSL | 706 |
| WP_010922251 | 703 | 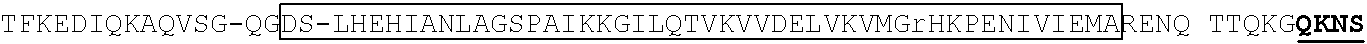 | 777 |
| WP_039695303 | 707 | PFKQIIQKSQVVG-DVDD-IEAVVHDLPGSPAIKKGILQSVKIVDELVKVMG-GNPDNIVIEMARENQ TTNRGRSQS | 780 |
| WP_045635197 | 702 | SFKEIIQKAQVIG-KTDD-VKQVVQELSGSPAIKKGILQSIKIVDELVKVMG-HAPESIVIEMARENQ TTARGKKNS | 775 |
| 5AXW_A | 427 | VPKKVDLSQQKEI---PT---TLVDDFILSPVVKRSFIQSIKVINAIIKKYG--LPNDIIIELAREKN --------S | 487 |
| WP_009880683 | 387 | TFKEDLQKAQVSG-QGDS-LHEHIANLAGSPAIKKGILQTVKVVDELVKVMGrHKPENIVIEMARENQ TTOKGQKNS | 461 |
| WP_010922251 | 703 | TFKEDIQKAQVSG-QGDS-LHEHIANLAGSPAIKKGILQTVKVVDELVKVMGrHKPENIVIEMARENQ TTOKGQKNS | 777 |
| WP_011054416 | 703 | TFKEDIQKAQVSG-QGDS-LHEHIANLAGSPAIKKGILQTVKVVDELVKVMGrHKPENIVIEMARENQ TTOKGQKNS | 777 |
| WP_011284745 | 703 | TFKEDIQKAQVSG-QGDS-LHEHIANLAGSPAIKKGILQTVKVVDELVKVMGrHKPENIVIEMARENQ TTQKGQKNS | 777 |
| WP_011285506 | 703 | TFKEDIQKAQVSG-QGDS-LHEHIANLAGSPAIKKGILQTVKVVDELVKVMGrHKPENIVIEMARENQ TTQKGQKNS | 777 |
| WP_011527619 | 703 | TFKEDIQKAQVSG-QGDS-LHEHIANLAGSPAIKKGILQTVKVVDELVKVMGrHKPENIVIEMARENQ TTOKGQKNS | 777 |
| WP_012560673 | 703 | TFKEDLQKAQVSG-QGDS-LHEHIANLAGSPAIKKGILQTVKVVDELVKVMGrHKPENIVIEMARENQ TTOKGQKNS | 777 |
| WP_014407541 | 703 | TFKEDIQKAQVSG-QGHS-LHEQIANLAGSPAIKKGILQTVKIVDELVKVMG-HKPENIVIEMARENQ TTQKGQKNS | 776 |
| WP_020905136 | 703 | TFKEDIQKAQVSG-QGDS-LHEHIANLAGSPAIKKGILQTVKVVDELVKVMGrHKPENIVIEMARENQ TTOKGQKNS | 777 |
| WP_023080005 | 703 | TFKEAIQKAQVSG-QGHS-LHEQIANLAGSPAIKKGILQTVKIVDELVKVMG-HKPENIVIEMARENQ TTOKGQKNS | 776 |
| WP_023610282 | 703 | TFKEAIQKAQVSG-QGHS-LHEQIANLAGSPAIKKGILQTVKIVDELVKVMG-HKPENIVIEMARENQ TTQKGQKNS | 776 |
| WP_030125963 | 703 | TFKEDLQKAQVSG-QGDS-LHEHIANLAGSPAIKKGILQTVKVVDELVKVMGrHKPENIVIEMARENQ TTOKGQKNS | 777 |
| WP_030126706 | 703 | TFKEDIQKAQVSG-QGDS-LHEHIANLAGSPAIKKGILQTVKVVDELVKVMGrHKPENIVIEMARENQ TTOKGQKNS | 777 |
| WP_031488318 | 703 | TFKEDIQKAQVSG-QGDS-LHEHIANLAGSPAIKKGILQTVKVVDELVKVMGrHKPENIVIEMARENQ TTQKGQKNS | 777 |
| WP_032460140 | 703 | TFKEDLQKAQVSG-QGDS-LHEHIANLAGSPAIKKGILQTVKVVDELVKVMGrHKPENIVIEMARENQ TTQKGQKNS | 777 |
| WP_032461047 | 703 | TFKEDLQKAQVSG-QGDS-LHEHIANLAGSPAIKKGILQTVKVVDELVKVMGrHKPENIVIEMARENQ TTOKGQKNS | 777 |
| WP_032462016 | 703 | TFKEDIQKAQVSG-QGDS-LHEHIANLAGSPAIKKGILQTVKVVDELVKVMGrHKPENIVIEMARENQ TTOKGQKNS | 777 |
| WP_032462936 | 703 | TFKEDIQKAQVSG-QGDS-LHEHIANLAGSPAIKKGILQTVKVVDELVKVMGrHKPENIVIEMARENQ TTOKGQKNS | 777 |
| WP_032464890 | 703 | TFKEDIQKAQVSG-QGDS-LHEHIANLAGSPAIKKGILQTVKVVDELVKVMGrHKPENIVIEMARENQ TTOKGQKNS | 777 |
| WP_033888930 | 528 | TFKEDIQKAQVSG-QGDS-LHEHIANLAGSPAIKKGILQTVKVVDELVKVMGrHKPENIVIEMARENQ TTOKGQKNS | 602 |
| WP_038431314 | 703 | TFKEDIQKAQVSG-QGDS-LHEHIANLAGSPAIKKGILQTVKVVDELVKVMGrHKPENIVIEMARENQ TTQKGQKNS | 777 |
| WP_038432938 | 703 | TFKEAIQKAQVSG-QGHS-LHEQIANLAGSPAIKKGILQTVKIVDELVKVMG-HKPENIVIEMARENQ TTQKGQKNS | 776 |
| WP_038434062 | 703 | TFKEDIQKAQVSG-QGDS-LHEHIANLAGSPAIKKGILQTVKIVDELVKVMGrHKPENIVIEMARENQ TTQKGQKNS | 777 |
| BAQ51233 | 614 | TFKEDIQKAQVSG-QGDS-LHEHIANLAGSPAIKKGILQTVKVVDELVKVMGrHKPENIVIEMARENQ TTOKGQKNS | 688 |
| KGE60162 | | -------------------------------------------------------------------- --------- | |
| KGE60856 | | -------------------------------------------------------------------- --------- | |
| WP_002989955 | 703 | TFKEDIQKAQVSG-QGDS-LHEHIANLAGSPAIKKGILQTVKVVDELVKVMGrHKPENIVIEMARENQ TTQKGQKNS | 777 |
| WP_003030002 | 704 | SFKEEIARAQIIG-DVDD-IANVVHDLPGSPAIKKGILQSVKIVDELVKVMG-HNPANIIIEMARENQ MTDKGRRNS | 777 |
| WP_003065552 | 707 | PFKQIIQKSQVVG-DVDD-IEAVVHDLPGSPAIKKGILQSVKIVDELVKVMG-DNPDNIVIEMARENQ TTNRGRSQS | 780 |
| WP_001040076 | 702 | SFKPIIDKARTGS-HSDN-LKEVIGELAGSPAIKKGILQSLKIVDELVKVMG-YEPEQIVVEMARENQ TTAKGLSRS | 775 |
| WP_001040078 | 705 | SFKSIISKAQAGS-HSDN-LKEVVGELAGSPAIKKGILQSLKIVDELVKVMG-YEPEQIVVEMARENQ TTNQGRRNS | 778 |
| WP_001040080 | 705 | SFKSIISKAQAGS-HSDN-LKEVVGELAGSPAIKKGILQSLKIVDELVKVMG-YEPEQIVVEMARENQ TTNQGRRNS | 778 |
| WP_001040081 | 705 | SEKSIISKAQAGS-HSDN-LKEVVGELAGSPAIKKGILQSLKIVDELVKVMG-YEPEQIVVEMARENQ TTNQGRRNS | 778 |
| WP_001040083 | 705 | SEKSIISKAQAGS-HSDN-LKEVVGELAGSPAIKKGILQSLKIVDELVKVMG-YEPEQIVVEMARENQ TTNQGRRNS | 778 |
| WP_001040085 | 705 | SFKSIISKAQAGS-HSDN-LKEVVGELAGSPAIKKGILQSLKIVDELVKVMG-YEPEQIVVEMARENQ TTNQGRRNS | 778 |
| WP_001040087 | 705 | SFKSIISKAQAGS-HSDN-LKEVVGELAGSPAIKKGILQSLKIVDELVKVMG-YEPEQIVVEMARENQ TTNQGRRNS | 778 |
| WP_001040088 | 705 | SEKSIISKAQAGS-HSDN-LKEVVGELAGSPAIKKGILQSLKIVDELVKVMG-YEPEQIVVEMARENQ TTNQGRRNS | 778 |
| WP_001040089 | 705 | SFKSIISKAQAGS-HSDN-LKEVVGELAGSPAIKKGILQSLKIVDELVKVMG-YEPEQIVVEMARENQ TTNQGRRNS | 778 |
| WP_001040090 | 705 | SFKSIISKAQAGS-HSDN-LKEVVGELAGSPAIKKGILQSLKIVDELVKVVG-YEPEQIVVEMARENQ TTNQGRRNS | 778 |
| WP_001040091 | 705 | SEKSIISKAQAGS-HSDN-LKEVVGELAGSPAIKKGILQSLKIVDELVKVMG-YEPEQIVVEMARENQ TTNQGRRNS | 778 |
| WP_001040092 | 705 | SFKSIISKAQSGS-HSDN-LKEVVGELAGSPAIKKGILQSLKIVDELVKVMG-YEPEQIVVEMARENQ TTNQGRRNS | 778 |
| WP_001040094 | 702 | SFKPIIDKARTGS-HSDN-LKEVVGELAGSPAIKKGILQSLKIVDELVKVMG-YEPEQIVVEMARENQ TTAKGLSRS | 775 |
| WP_001040095 | 702 | SFKPIIDKARTGS-HLDN-LKEVVGELAGSPAIKKGILQSLKIVDELVKVMG-YEPEQIVVEMARENQ TTAKGLSRS | 775 |
| WP_001040096 | 702 | SFKPIIDKARTGS-HLDN-LKEVVGELAGSPAIKKGILQSLKIVDELVKVMG-YEPEQIVVEMARENQ TTAKGLSRS | 775 |
| WP_001040097 | 702 | SFKPIIDKARTGS-HSDN-LKEVVGELAGSPAIKKGILQSLKIVDELVKVMG-YEPEQIVVEMARENQ TTAKGLSRL | 775 |
| WP_001040098 | 702 | SFKPIIDKARTGS-HSDN-LKEVVGELAGSPAIKKGILQSLKIVDELVKVMG-YEPEQIVVEMARENQ TTAKGLSRS | 775 |
| WP_001040099 | 702 | SFKPIIDKARTGS-HSDN-LKEVVGELAGSPAIKKGILQSLKIVDELVKVMG-YEPEQIVVEMARENQ TTAKGLSRS | 775 |
| WP_001040100 | 702 | SFKPIIDKARTGS-HSDN-LKEVVGELAGSPAIKKGILQSLKIVDELVKVMG-YEPEQIVVEMARENQ TTAKGLSRS | 775 |
| WP_001040104 | 705 | SFKSIISKAQAGS-HSDN-LKEVVGELAGSPAIKKGILQSLKIVDELVKVMG-YEPEQIVVEMARENQ TTNQGRRNS | 778 |
| WP_001040105 | 705 | SEKSIISKAQAGS-HSDN-LKEVVGELAGSPAIKKGILQSLKIVDELVKVMG-YEPEQIVVEMARENQ TTNQGRRNS | 778 |
| WP_001040106 | 705 | SFKPIIDKAQAGS-HSDN-LKEVVGELAGSPAIKKGILQSLKIVDELVKVMG-YEPEQVVVEMARENQ TTNQGRRNT | 778 |
| WP_001040107 | 705 | SFKPIIDKAQAGS-HSDN-LKEVVGELAGSPAIKKGILQSLKIVDELVKVMG-YEPEQVVVEMARENQ TTNQGRRNT | 778 |
| WP_001040108 | 705 | SFKPIIDKAQAGS-HSDN-LKEVVGELAGSPAIKKGILQSLKIVDELVKVMG-YEPEQVVVEMARENQ TTNQGRRNT | 778 |
| WP_001040109 | 705 | SFKPIIDKAQAGS-HSDN-LKEVVGELAGSPAIKKGILQSLKIVDELVKVMG-YEPEQVVVEMARENQ TTNQGRRNT | 778 |
| WP_001040110 | 705 | SFKPIIDKAQAGS-HSDN-LKEVVGELAGSPAIKKGILQSLKIVDELVKVMG-YEPEQVVVEMARENQ TTNQGRRNT | 778 |
| WP_015058523 | 705 | SFKSIISKAQSGS-HSDN-LKEVVGELAGSPAIKKGILQSLKIVDELVKVMG-YEPEQIVVEMARENQ TTNQGRRNS | 778 |
| WP_017643650 | 702 | SFKPIIDKARTGS-HSDN-LKEVVGELAGSPAIKKGILQSLKIVDELVKVMG-YEPEQIVVEMARENQ TTAKGLSRL | 775 |
| WP_017647151 | 705 | SFKSIISKAQAGS-HSDN-LKEVVGELAGSPAIKKGILQSLKIVDELVKVMG-YEPEQIVVEMARENQ TTNQGRRNS | 778 |
| WP_017648376 | 705 | SFKSIISKAQAGS-HSDN-LKEVVGELAGSPAIKKGILQSLKIVDELVKVMG-YEPEQIVVEMARENQ TTNQGRRNS | 778 |
| WP_017649527 | 705 | SFKSIISKAQAGS-HSDN-LKEVVGELAGSPAIKKGILQSLKIVDELVKVMG-YEPEQIVVEMARENQ TINQGRRNS | 778 |
| WP_017771611 | 705 | SFKPIIDKAQAGS-HSDN-LKEVVGELAGSPAIKKGILQSLKIVDELVKVMG-YEPEQVVVEMARENQ TTNQGRRNT | 778 |
| WP_017771984 | 705 | SEKSIISKAQAGS-HSDN-LKEVVGELAGSPAIKKGILQSLKIVDELVKVMG-YEPEQIVVEMARENQ TTNQGRRNS | 778 |
| CFQ25032 | 705 | SFKSIISKAQAGS-HSDN-LKEVVGELAGSPAIKKGILQSLKIVDELVKVMG-YEPEQIVVEMARENQ TTNQGRRNS | 778 |
| CFV16040 | 705 | SEKSIISKAQAGS-HSDN-LKEVVGELAGSPAIKKGILQSLKIVDELVKVMG-YEPEQIVVEMARENQ TTNQGRRNS | 778 |
| KLJ37842 | 705 | SFKSIISKAQAGS-HSDN-LKEVVGELAGSPAIKKGILQSLKIVDELVKVMG-YEPEQIVVEMARENQ TTNQGRRNS | 778 |
| KLJ72361 | 705 | SFKSIISKAQAGS-HSDN-LKEVVGELAGSPAIKKGILQSLKIVDELVKVMG-YEPEQIVVEMARENQ TTNQGRRNS | 778 |
| KLL20707 | 719 | SFKSIISKAQAGS-HSDN-LKEVVGELAGSPAIKKGILQSLKIVDELVKVMG-YEPEQIVVEMARENQ TTNQGRRNS | 792 |
| KLL42645 | 705 | SFKPIIDKAQAGS-HSDN-LKEVVGELAGSPAIKKGILQSLKIVDELVKVMG-YEPEQVVVEMARENQ TTNKGRRNT | 778 |
| WP_047207273 | 705 | SFKSIISKAQAGS-HSDN-LKEVVGELAGSPAIKKGILQSLKIVDELVKVMG-YEPEQIVVEMARENQ TTNQGRRNS | 778 |
| WP_047209694 | 702 | SFKPIIDKARTGS-HSDN-LKEVVGELAGSPAIKKGILQSLKIVDELVKVMG-YEPEQIVVEMARENQ TTAKGLSRS | 775 |
| WP_050198062 | 705 | SEKSIISKAQAGS-HSDN-LKEVVGELAGSPAIKKGILQSLKIVDELVKVMG-YEPEQIVVEMARENQ TTNQGRRNS | 778 |
| WP_050201642 | 705 | SFKSIISKAQAGS-HSDN-LKEVVGELAGSPAIKKGILQSLKIVDELVKVMG-YEPEQIVVEMARENQ TTNQGRRNS | 778 |
| WP_050204027 | 705 | SFKPIIDKAQAGS-HSDN-LKEVVGELAGSPAIKKGILQSLKIVDELVKVMG-YEPEQVVVEMARENQ TTNQGRRNT | 778 |
| WP_050881965 | 705 | SEKSIISKAQAGS-HSDN-LKEVVGELAGSPAIKKGILQSLKIVDELVKVMG-YEPEQIVVEMARENQ TTNQGRRNS | 778 |
| WP_050886065 | 705 | SEKSIISKAQAGS-HSDN-LKEVVGELAGSPAIKKGILQSLKIVDELVKVMG-YEPEQIVVEMARENQ TTNQGRRNS | 778 |
| AHN30376 | 705 | SFKSIISKAQSGS-HSDN-LKEVVGELAGSPAIKKGILQSLKIVDELVKVMG-YEPEQIVVEMARENQ TTNQGRRNS | 778 |
| EAO78426 | 705 | SFKSIISKAQAGS-HSDN-LKEVVGELAGSPAIKKGILQSLKIVDELVKVMG-YEPEQIVVEMARENQ TTNQGRRNS | 778 |
| CCW42055 | 705 | SFKSIISKAQSGS-HSDN-LKEVVSELAGSPAIKKGILQSLKIVDELVKVMG-YKPEQIVVEMARENQ TTNQGRRNS | 778 |
| WP_003041502 | 704 | SFKEEIAKAQIIG-DVDD-IANVVHDLPGSPAIKKGILQSVKIVDELVKVMG-HNPANIIIEMARENQ TTDRGRRNS | 777 |
| WP_037593752 | 705 | SFKEEIARAQIIG-DVDD-IANVVHDLPGSPAIKKGILQSVKIVDELVKVMG-HNPANIIIEMARENQ TTDKGRRNS | 778 |
| WP_049516684 | 705 | SFKEEIARAQIIG-DVDD-IANVVHDLPGSPAIKKGILQSVKIVDELVKVMG-HNPANIIIEMARENQ TTDKGRRNS | 778 |
| GAD46167 | 704 | SFKEEIARAQIIG-DVDD-IANVVHDLPGSPAIKKGILQSVKIVDELVKVMG-HNPANIIIEMARENQ TTDKGRRNS | 777 |
| WP_018363470 | 705 | SFKQIIQEAQVVG-DVDD-IETVVHDLPGSPAIKKGILQSVKIVDELIKVMG-DNPDNIVIEMARENQ TTNRGRSQS | 778 |
| WP_003043819 | 713 | TFKEEIEKAQVSG-QGDS-LHEQIADLAGSPAIKKGILQTVKIVDELVKVMG-HKPENIVIEMARENQ TTTKGLQQS | 786 |
| WP_006269658 | 704 | SFKEEIARAQIID-DVDD-IANVVHDLPGSPAIKKGILQSVKIVDELVKVMG-HNPANIIIEMARENQ TTDKGRRNS | 777 |
| WP_048800889 | 704 | PFKQIIKDAQAID-DVDD-IELIVHDLPGSPAIKKGILQSIKIVDELVKVMG-YNPDNIVIEMARENQ TTTKGRRNS | 777 |
| WP_012767106 | 703 | TFKEAIQKAQVSG-QGHS-LHEQIANLAGSPAIKKGILQSVKVVDELVKVMG-HKPENIVIEMARENQ TTQKGQKNS | 776 |
| WP_014612333 | 703 | TFKEAIQKAQVSG-QGHS-LHEQIANLAGSPAIKKGILQSVKVVDELVKVMG-HKPENIVIEMARENQ TTOKGQKNS | 776 |
| WP_015017095 | 703 | TFKEAIQKAQVSG-QGHS-LHEQIANLAGSPAIKKGILQSVKVVDELVKVMG-HKPENIVIEMARENQ TTOKGQKNS | 776 |
| WP_015057649 | 703 | TFKEAIQKAQVSG-QGHS-LHEQIANLAGSPAIKKGILQSVKVVDELVKVMG-HKPENIVIEMARENQ TTOKGQKNS | 776 |
| WP_048327215 | 703 | TFKEAIQKAQVSG-QGHS-LHEQIANLAGSPAIKKGILQSVKVVDELVKVMG-HKPENIVIEMARENQ TTQKGQKNS | 776 |
| WP_049519324 | 703 | TFKEAIQKAQVSG-QGHS-LHEQIANLAGSPAIKKGILQSVKVVDELVKVMG-HKPENIVIEMARENQ TTOKGQKNS | 776 |
| WP_012515931 | 703 | SFIDEIAKAQVIG-KTEY-SKDLVGNLAGSPAIKKGISQTIKIVDELVKIMG-YLPQQIVIEMARENQ TTAQGIKNA | 776 |
| WP_021320964 | 703 | SFIDEIAKAQVIG-KTEY-SKDLVGNLASSPAIKKGISQTIKIVDELVKIMG-YLPQQIVIEMARENQ TTAQGIKNA | 776 |
| WP_037581760 | 703 | SFIDEIAKAQVIG-KTEY-SKDLVGNLAGSPAIKKGISQTIKIVDELVKIMG-YLPQQIVIEMARENQ TTAQGIKNA | 776 |
| WP_004232481 | 704 | SFKTTIQEAQVVG-DVDD-IEAVVHDLPGSPAIKKGILQSVKIVDELVKVMG-HNPQNIVIEMARENQ ITGYGRNRS | 777 |
| WP_009854540 | 705 | PFKQIIQKSQVVG-DVDD-IEAVVHDLPGSPAIKKGILQSVKIVDELVKVMG-DNPDNIVIEMARENQ TTNRGRSQS | 778 |
| WP_012962174 | 705 | PFKQIIKDAQIIG-DIDD-VTSVVRELPGSPAIKKGILQSVKIVDELVKVMG-HNPDNIVIEMARENQ TTNRGRNOS | 778 |
| WP_039695303 | 707 | PFKQIIQKSQVVG-DVDD-IEAVVHDLPGSPAIKKGILQSVKIVDELVKVMG-GNPDNIVIEMARENQ TTNRGRSQS | 780 |
| WP_014334983 | 704 | SFKTIIQEAQVVG-DVDD-IEAVVHDLPGSPAIKKGILQSVKIVDELVKVMG-DNPDNIVIEMARENQ TTGYGRNKS | 777 |
| WP_003099269 | 703 | DFAKIIKNEQEKTiKNES-LEETIANLAGSPAIKKGILQSIKIVDEIVKIMG-QNPDNIVIEMARENQ STMQGIKNS | 777 |
| AHY15608 | 703 | DFAKIIKNEQEKTiKNES-LEETIANLAGSPAIKKGILQSIKIVDEIVKIMG-QNPDNIVIEMARENQ STMQGIKNS | 777 |
| AHY17476 | 703 | DFAKIIKNEQEKTiKNES-LEETIANLAGSPAIKKGILQSIKIVDEIVKIMG-QNPDNIVIEMARENQ STMQGIKNS | 777 |
| ESR09100 | | -------------------------------------------------------------------- --------- | |
| AGM98575 | 703 | DFAKIIKNEQEKTiKNES-LEETIANLAGSPAIKKGILQSIKIVDEIVKIMG-QNPDNIVIEMARENQ STMQGIKNS | 777 |
| ALF27331 | 704 | SFKEEIAKAQVIG-ETDN-LNQVVSDIAGSPAIKKGILQSLKIVDELVKIMG-HQPENIVVEMARENQ FTNQGRRNS | 777 |
| WP_018372492 | 717 | DFKEIIRKAQTIE-NIDT-NQALVSSLPGSPAIKKGILQSLNIVDEIIAIMG-YAPTNIVIEMARENQ TTQKGRDNS | 790 |
| WP_045618028 | 703 | SFKEIIQKAQVVG-KIND-VKQVVQELPGSPAIKKGILQSIKLVDELVKVMG-HAPESIVIEMARENQ TTARGKKNS | 776 |
| WP_045635197 | 702 | SFKEIIQKAQVIG-KTDD-VKQVVQELSGSPAIKKGILQSIKIVDELVKVMG-HAPESIVIEMARENQ TTARGKKNS | 775 |
| WP_002263549 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002263887 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002264920 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002269043 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002269448 | 704 | SFKEEIAKAQVIG-ETDN-LNQVVSDIAGSPAIKKGILQSLKIVDELVKIMG-HQPENIVVEMARENQ FTNQGRONS | 777 |
| WP_002271977 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002272766 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002273241 | 704 | SFKEEIAKAQVIG-ETDN-LNQVVSDIAGSPAIKKGILQSLKIVDELVKIMG-HQPENIVVEMARENQ FTKQGRRNS | 777 |
| WP_002275430 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002276448 | 704 | SFKEEIAKAQVIG-ETDN-LNQVVSDIAGSPAIKKGILQSLKIVDELVKIMG-HQPENIVVEMARENQ FTKQGRRNS | 777 |
| WP_002277050 | 705 | TFKDDIVKAQYVD-NSDD-LHQVVQSLAGSPAIKKGILQSLKIVDELVKVMG-KEPEQIVVEMARENQ TTAKGRRNS | 778 |
| WP_002277364 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002279025 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002279859 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002280230 | 704 | SFKEEIAKAQVIG-ETDN-LNQVVSDIAGSPAIKKGILQSLKIVDELVKIMG-HQPENIVVEMARENQ FTKQGRRNS | 777 |
| WP_002281696 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002282247 | 705 | TFKDDIVKAQYVD-NSDD-LHQVVQSLAGSPAIKKGILQSLKIVDELVKVMG-KEPEQIVVEMARENQ TTAKGRRNS | 778 |
| WP_002282906 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002283846 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002287255 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002288990 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002289641 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002290427 | 704 | SFKEEIAKAQVIG-ETDN-LNQVVSDIAGSLAIKKGILQSLKIVDELVKIMG-HQPENIVVEMARENQ FTNQGRRNS | 777 |
| WP_002295753 | 704 | SFKEEIAKAQVIG-ETDN-LNQVVSDIAGSPAIKKGILQSLKIVDELVKIMG-HQPENIVVEMARENQ FTKQGRRNS | 777 |
| WP_002296423 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002304487 | 714 | SFKEEIAKAQVIG-EMDG-LNQVVSDIAGSPAIKKGILQSLKIVDELVKVMG-HNPANIVIEMARENQ TTAKGRRSS | 787 |
| WP_002305844 | 704 | SFKEEIAKAQVIG-ETDN-LNQVVSDIAGSPAIKKGILQSLKIVDELVKIMG-HQPENIVVEMARENQ FTKQGRRNS | 777 |
| WP_002307203 | 704 | SFKEEIAKAQVIG-ETDN-LNQVVSDIAGSPAIKKGILQSLKIVDELVKIMG-HQPENIVVEMARENQ FTNQGRRNS | 777 |
| WP_002310390 | 704 | SFKEEIAKAQVIG-ETDN-LNQVVSDIAGSLAIKKGILQSLKIVDELVKIMG-HQPENIVVEMARENQ FTNQGRRNS | 777 |
| WP_002352408 | 704 | SFKEEIAKAQVIG-ETDN-LNQVVSDIAGSPAIKKGILQSLKIVDELVKIMG-HQPENIVVEMARENQ FTNOGORNS | 777 |
| WP_012997688 | 704 | SFKEEIAKAQVIG-ETDN-LNQVVSDIAGSPAIKKGILQSLKIVDELVKIMG-HQPENIVVEMARENQ FTNQGRRNS | 777 |
| WP_014677909 | 704 | SFKEEIAKAQVIG-ETDN-LNQVVSDIAGSPAIKKGILQSLKIVDELVKIMG-HQPENIVVEMARENQ FTNQGRRNS | 777 |
| WP_019312892 | 704 | SFKEEIAKAQVIG-ETDN-LNQVVSDIAGSPAIKKGILQSLKIVDELVKIMG-HQPENIVVEMARENQ FTNQGRRNS | 777 |
| WP_019313659 | 704 | SFKEEIAKAQVIG-ETDN-LNQVVSDIAGSPAIKKGILQSLKIVDELVKIMG-HQPENIVVEMARENQ FTNQGRRNS | 777 |
| WP_019314093 | 704 | SFKEEIAKAQVIG-ETDN-LNQVVSDIAGSPAIKKGILQSLKIVDELVKIMG-HQPENIVVEMARENQ FTNQGRRNS | 777 |
| WP_019315370 | 704 | SFKEEIAKAQVIG-ETDN-LNQVVSDIAGSPAIKKGILQSLKIVDELVKIMG-HQPENIVVEMARENQ FTNQGRRNS | 777 |
| WP_019803776 | 704 | SFKEEIAKAQVIG-ETDN-LNQVVSDIAGSPAIKKGILQSLKIVDELVKIMG-HQPENIVVEMARENQ FTNQGRRNS | 777 |
| WP_019805234 | 704 | SFKEEIAKAQVIG-ETDN-LNQVVSDIAGSLAIKKGILQNLKIVDELVKIMG-HQPENIVVEMARENQ FTNQGRRNS | 777 |
| WP_024783594 | 704 | SFKEEIAKAQVIG-ETDN-LNQVVSDIAGSPAIKKGILQSLKIVDELVKIMG-HQPENIVVEMARENQ FTNQGRRNS | 777 |
| WP_024784288 | 705 | TFKDDIVKAQYVD-NSDD-LHQVVQSLAGSPAIKKGILQSLKIVDELVKVMG-KEPEQIVVEMARENQ TTAKGRRNS | 778 |
| WP_024784666 | 704 | SFKEEIAKAQVIG-ETDN-LNQVVSDIAGSPAIKKGILQSLKIVDELVKIMG-HQPENIVVEMARENQ FTNQGRRNS | 777 |
| WP_024784894 | 704 | SFKEEIAKAQVIG-ETDN-LNQVVSDIAGSPAIKKGILQSLKIVDELVKIMG-HQPENIVVEMARENQ FTNQGRRNS | 777 |
| WP_024786433 | 705 | TFKDDIVKAQYVD-NSDD-LHQVVQSLAGSPAIKKGILQSLKIVDELVKVMG-KEPEQIVVEMARENQ TTAKGRRNS | 778 |
| WP_049473442 | 704 | SFKEEIAKAQVIG-ETDN-LNQVVSDIAGSPAIKKGILQSLKIVDELVKIMG-HQPENIVVEMARENQ FTNQGRRNS | 777 |
| WP_049474547 | 704 | SFKEEIAKAQVIG-ETDN-LNQVVSDIAGSPAIKKGILQSLKIVDELVKIMG-HQPENIVVEMARENQ FTNQGRRNS | 777 |
| EMC03581 | 697 | SFKEEIAKAQVIG-ETDN-LNQVVSDIAGSPAIKKGILQSLKIVDELVKIMG-HQPENIVVEMARENQ FTNQGRRNS | 770 |
| WP_000428612 | 705 | SFKEIIKKAQVVG-KTDD-VKQVVQELPGSPAIKKGILQSIKLVDELVKVMG-HEPESIVIEMARENQ TTARGKKNS | 778 |
| WP_000428613 | 703 | SFKEITQKAQVVG-KTDD-VKQVVQELPGSPAIKKGILQSIKIVDELVKVMG-HTPESIVIEMARENQ TTARGKKNS | 776 |
| WP_049523028 | 702 | SFKETIQKAQVVG-ETND-VKQVVQELPGSPAIKKGILQSIKIVDELVKVMG-HAPESVVIEMARENO TTNKGKSKS | 775 |
| WP_003107102 | 672 | DFASIIKEAQEKTIKSEK-LEETIANLAGSPAIKKGILQSVKIVDEVVKVMG-YEPSNIVIEMARENQ STORGINNS | 746 |
| WP_054279288 | 705 | SFKEEIKKAQEGG-LKDS-INDQIRDLAGSPAIKKGILQTINIVDEIVKIMG-KAPQHIVVEMARDVQ KTDIGVKQS | 778 |
| WP_049531101 | 703 | SFKEIIQESQVVG-KPDD-VKQIVQELPGSSAIKKGILQSIKLVDELVKVMG-HDPESIVIEMARENQ TTARGKKNS | 776 |
| WP_049538452 | 703 | SFKEIIQKAQVFG-KIND-VKQVVQELPGSPAIKKGILQSIKIVEELVKVMG-HEPESIVIEMARENQ TTTRGKKNS | 776 |
| WP_049549711 | 703 | SFKKIIQKSQVVG-ETDD-VKQVVRELPGSPAIKKGILQSIKIVDELVKVMD-HAPESIVIEMARENQ TTARGKKNS | 776 |
| WP_007896501 | 706 | DFEKLIDDAQKKAIKRES-LTEAVANLAGSPAIKKGILQSLKVVDEIVKVMG-HNPDNIVIEMSRENQ TTAQGLKNA | 780 |
| EFR44625 | 658 | DFEKLIDDAQKKAiKRES-LTEAVANLAGSPAIKKGILQSLKVVDEIVKVMG-HNPDNIVIEMSRENQ TTAQGLKNA | 732 |
| WP_002897477 | 702 | SFKEIIQKAQVVG-KTDD-VKQVVQELPGSPAIKKGILQSIKIVDELVKVMG-YALESIVIEMARENQ TTARGKKNS | 775 |
| WP_002906454 | 702 | SFKEIIQKAQVVG-KTDD-VKQVVQEIPGSPAIKKGILQSIKIVDELVKVMG-HNPESIVIEMARENQ TTAKGKKNS | 775 |
| WP_009729476 | 703 | SFKEIIQKAQVVG-KIND-VKQVVQELPGSPAIKKGILQSIKIVDELVKVMG-HAPESIVIEMARENQ TTARGKKNS | 776 |
| COR24647 | 706 | SFKDEIANSQVIG-DGDD-LHQVVQELAGSPAIKKGILQSLKIVDELVKVMG-YNPEQIVVEMARENQ TTARGRNNS | 779 |
| WP_000066813 | 705 | SFKEIIQKAQVFG-KTND-VKQVVQELPGSPAIKKGILQSIKIVDELVKVMG-HAPESIVIEMARENQ TTARGKKNS | 778 |
| WP_009754323 | 703 | SFKEIIQKAQVVG-KTDD-LTQVVRELSGSPAIKKGILQSIKIVDELVKIMG-YAPESIVIEMARENQ TTAKGKKNS | 776 |
| WP_044674937 | 702 | SFVDEIRLAQGSG-EAED-YRAEVONLAGSPAIKKGILQSLKIVDELIEVMG-YDPEHIVVEMARENQ FTNQGRRNS | 775 |
| WP_044676715 | 704 | SFVDEIRLAQGSG-EAED-YRAEVONLAGSPAIKKGILQSLKIVDELIEVMG-YDPEHIVVEMARENQ FTNQGRRNS | 777 |
| WP_044680361 | 704 | SFVDEIRLAQGSG-EAED-YRAEVONLAGSPAIKKGILQSLKIVDELIEVMG-YDPEHIVVEMARENQ FTNQGRRNS | 777 |
| WP_044681799 | 702 | SFVDEIRLAQGSG-EAED-YRAEVONLAGSPAIKKGILQSLKIVDELIEVMG-YDPEHIVVEMARENQ FTNQGRRNS | 775 |
| WP_049533112 | 704 | SFKEEIAKAQVIG-ETDD-LNQVVSDIAGSPAIKKGILQSLKIVDELVKVMG-YNPANIVIEMARENQ TTDKGRRNS | 777 |
| WP_029090905 | 683 | LFKKIIEEQFENEtALLN--KORIDELAASPANKKGIWQAIKIVKELEKVLQ-QPAENIFIEFARSDE ES----KRS | 752 |
| WP_006506696 | 706 | GYAQMIEEATSCPeDGKF-TYEEVERLAGSPALKRGIWQSLQIVEEITKVMK-CRPKYIYIEFERSEE -----KERT | 776 |
| AIT42264 | 703 | TFKEDIQKAQVSG-QGDS-LHEHIANLAGSPAIKKGILQTVKVVDELVKVMGrHKPENIVIEMARENQ TTOKGQKNS | 777 |
| WP_034440723 | 711 | SFKEKIRKAQDIN-QVND-IKEIVKDLPGSPAIKKGIYQSIRIVDEIIRKMK-DRPKNIVIEMARENQ TTQEGKNKS | 784 |
| AKQ21048 | 703 | TFKEDIQKAQVSG-QGDS-LHEHIANLAGSPAIKKGILQTVKVVDELVKVMGrHKPENIVIEMARENQ TTOKGQKNS | 777 |
| WP_004636532 | 707 | SFKKKIEDAQTIE-DTTH-IYDTVAELPGSPAIKKGIRQALKIVEEIIDIIG-YEPENIVVEMARESQ TTKKGKDLS | 780 |
| WP_002364836 | 715 | SFKNAIQKAQSSE-HEET-LSETVNELAGSPAIKKGIYQSLKIVDELVAIMG-YAPKRIVVEMARENQ TTSTGKRRS | 788 |
| WP_016631044 | 666 | SFKNAIQKAQSSE-HEET-LSETVNELAGSPAIKKGIYQSLKIVDELVAIMG-YAPKRIVVEMARENQ TTSTGKRRS | 739 |
| EMS75795 | 455 | SFKEELANELALA-GNOS-LLEVVEALLGSPAIKKGIWOTLKIVEELIEIIG-YNPKNIVVEMARENQ RT----NRS | 524 |
| WP_002373311 | 715 | SFKNAIQKAQSSE-HEET-LSETVNELAGSPAIKKGIYQSLKIVDELVAIMG-YAPKRIVVEMARENQ TTSTGKRRS | 788 |
| WP_002378009 | 715 | SFKNAIQKAQSSE-HEET-LSETVNELAGSPAIKKGIYQSLKIVDELVAIMG-YAPKRIVVEMARENQ TTSTGKRRS | 788 |
| WP_002407324 | 715 | SFKNAIQKAQSSE-HEET-LSETVNELAGSPAIKKGIYQSLKIVDELVAIMG-YAPKRIVVEMARENQ TTSTGKRRS | 788 |
| WP_002413717 | 715 | SFKNAIQKAQSSE-HEET-LSETVNELAGSPAIKKGIYQSLKIVDELVAIMG-YAPKRIVVEMARENQ TTSTGKRRS | 788 |
| WP_010775580 | 717 | SFKNAIQKAQSSE-HEET-LSETVNELAGSPAIKKGIYQSLKIVDELVAIMG-YAPKRIVVEMARENQ TTSTGKRRS | 790 |
| WP_010818269 | 715 | SFKNAIQKAQSSE-HEET-LSETVNELAGSPAIKKGIYQSLKIVDELVAIMG-YAPKRIVVEMARENQ TTSTGKRRS | 788 |
| WP_010824395 | 715 | SFKNAIQKAQSSE-HEET-LSETVNELAGSPAIKKGIYQSLKIVDELVAIMG-YAPKRIVVEMARENQ TTSTGKRRS | 788 |
| WP_016622645 | 715 | SFKNAIQKAQSSE-HEET-LSETVNELAGSPAIKKGIYQSLKIVDELVAIMG-YAPKRIVVEMARENQ TTSTGKRRS | 788 |
| WP_033624816 | 715 | SFKNAIQKAQSSE-HEET-LSETVNELAGSPAIKKGIYQSLKIVDELVAIMG-YAPKRIVVEMARENQ TTSTGKRRS | 788 |
| WP_033625576 | 715 | SFKNAIQKAQSSE-HEET-LSETVNELAGSPAIKKGIYQSLKIVDELVAIMG-YAPKRIVVEMARENQ TTSTGKRRS | 788 |
| WP_033789179 | 715 | SFKNAIQKAQSSE-HEET-LSETVNELAGSPAIKKGIYQSLKIVDELVAIMG-YAPKRIVVEMARENQ TTSTGKRRS | 788 |
| WP_002310644 | 715 | SFKKEIKKAQMIT-DTEN-LEEIVKELTGSPAIKKGILQSLKIVDEIVGIMG-YEPANIVVEMARENQ TTGRGLKSS | 788 |
| WP_002312694 | 716 | SFKKEIKKAQMIT-DTEN-LEEIVKELTGSPAIKKGILQSLKIVDEIVGIMG-YEPANIVVEMARENQ TTGRGLKSS | 789 |
| WP_002314015 | 716 | SFKKEIKKAQMIT-DTEN-LEEIVKELTGSPAIKKGILQSLKIVDEIVGIMG-YEPANIVVEMARENQ TTGRGLKSS | 789 |
| WP_002320716 | 716 | SFKKEIKKAQMIT-DTEN-LEEIVKELTGSPAIKKGILQSLKIVDEIVGIMG-YEPANIVVEMARENQ TTGRGLKSS | 789 |
| WP_002330729 | 715 | SFKKEIKKAQMIT-DTEN-LEEIVKELTGSPAIKKGILQSLKIVDEIVGIMG-YEPANIVVEMARENQ TTGRGLKSS | 788 |
| WP_002335161 | 716 | SFKKEIKKAQMIT-DTEN-LEEIVKELTGSPAIKKGILQSLKIVDEIVGIMG-YEPANIVVEMARENQ TTGRGLKSS | 789 |
| WP_002345439 | 716 | SFKKEIKKAQMIT-DTEN-LEEIVKELTGSPAIKKGILQSLKIVDEIVGIMG-YEPANIVVEMARENQ TTGRGLKSS | 789 |
| WP_034867970 | 711 | SFKEEIAKATVFS-KHKS-LVDVIQDLPGSPAIKKGIWQSLKIVEELIAIIG-YKPKNIVIEMARENQ KT----HRT | 780 |
| WP_047937432 | 716 | SFKKEIKKAQMIT-DTEN-LEEIVKELTGSPAIKKGILQSLKIVDEIVGIMG-YEPANIVVEMARENQ TTGRGLKSS | 789 |
| WP_010720994 | 711 | SFKEEIAKATVFS-KHKS-LVDVIQDLPGSPAIKKGIWQSLKIVEELIAIIG-YKPKNIVIEMARENQ KT----HRT | 780 |
| WP_010737004 | 711 | SFKEEIAKATVFS-KHKS-LVDVIQDLPGSPAIKKGIWQSLKIVEELIAIIG-YKPKNIVIEMARENQ KT----HRT | 780 |
| WP_034700478 | 711 | SFKEEIAKATVFS-KHKS-LVDVIQDLPGSPAIKKGIWQSLKIVEELIAIIG-YKPKNIVIEMARENQ KT----HRT | 780 |
| WP_007209003 | 708 | SFKKIIEDSQPYK-EQQS-AEEIVSELSGSPAIKKGILQSLKIVDELVAIMG-YKPKNIVVEMARENQ TTGRGKONS | 781 |
| WP_023519017 | 705 | SFKETIANELIMS-DSNV-LLDQVKAIPGSPAVKKGIWQSIKIVEEIIGIIG-KAPKNIVIEMARENQ RTSR----S | 774 |
| WP_010770040 | 708 | SFKSEIAEAQSDM-NTED-LHEVVONLAGSPAIKKGILQSLKIVDELVDIMG-SLPKNIVVEMARENQ TTSRGRINS | 781 |
| WP_048604708 | 704 | TFKEEIEKEQLKA-NSEESLIEIVONLAGSPAIKKGIFQSLKIVDELVEIMG-YAPTNIVVEMARENQ TTANGRRNS | 778 |
| WP_010750235 | 710 | SFKEEIAKELTLS-DKQS-LLEVVEAIPGSPAIKKGIWQTLKIVEELIAIIG-YKPKNIVIEMARENQ TTTGGKNRS | 783 |
| AII16583 | 742 | TFKEDIQKAQVSG-QGDS-LHEHIANLAGSPAIKKGILQTVKVVDELVKVMGrHKPENIVIEMARENQ TTOKGOKNS | 816 |
| WP_029073316 | 718 | GFKKTIDDANSTSVSGKF-SYAEVQELAGSPAIKRGIWQALLIVDEIKKIMK-HEPAHVYIEFARNED -----KERK | 788 |
| WP_031589969 | 718 | GFKKTIDDANSTSVSGKF-SYAEVQELAGSPAIKRGIWQALLIVDEIKKIMK-HEPAHVYIEFARNED -----KERK | 788 |
| KDA45870 | 699 | SFKETIKNAQVIE-KEET-LAKTVQELPGSPAIKKGILQSLEIVDEIIKVMG-YKPKSIVVEMARETQ --THGTRKR | 771 |
| WP_039099354 | 713 | DFDKLITEANQMM-LAENdVQDVINDLYTSPONKKALRQILLVVNDIQKAMKgQAPERILIEFAREDE VNPRLSVQR | 788 |
| AKP02966 | 707 | DFKNYIENHNLNKNEDQN-ISNLVNDIHVSPALKRGITQSIKIVQEIVKFMG-HAPKYIFIEVTRETK TTSRGKRIQ | 785 |
| WP_010991369 | 707 | SFKSIIEKEQVTT-ADKD-IQSIVADLAGSPAIKKGILQSLKIVDELVSVMG-YPPQTIVVEMARENQ TTGKGKNNS | 780 |
| WP_033838504 | 707 | SFKSIIEKEQVTT-ADKD-IQSIVADLAGSPAIKKGILQSLKIVDELVSVMG-YPPQTIVVEMARENQ TTGKGKNNS | 780 |
| EHN60060 | 710 | SFKSIIEKEQVTT-ADKD-IQSIVADLAGSPAIKKGILQSLKIVDELVSVMG-YPPQTIVVEMARENQ TTGKGKNNS | 783 |
| EFR89594 | 476 | SFKSIIEKEQVTT-ADKD-IQSIVADLAGSPAIKKGILQSLKIVDELVSVMG-YPPQTIVVEMARENQ TTGKGKNNS | 549 |
| WP_038409211 | 707 | SFKSIIEKEQVST-ADKG-IQSIVAELAGSPAIKKGILQSLKIVDELVGIMG-YPPQTIVVEMARENQ TTGKGKNNS | 780 |
| EFR95520 | 326 | SFKSIIEKEQVST-ADKG-IQSIVAELAGSPAIKKGILQSLKIVDELVGIMG-YPPQTIVVEMARENQ TTGKGKNNS | 399 |
| WP_003723650 | 707 | SFKSIIEKEQVST-TDKD-LQSIVAELAGSPAIKKGILQSLKIVDELVSIMG-YPPQTIVVEMARENQ TTGKGKNNS | 780 |
| WP_003727705 | 707 | SFKSIIEKEQVST-TDKD-LQSIVADLAGSPAIKKGILQSLKIVDELVSIMG-YPPQTIVVEMARENQ TTGKGKNNS | 780 |
| WP_003730785 | 707 | SFKSIIEKEQVST-TDKD-LOSIVADLAGSPAIKKGILQSLKIVDELVSIMG-YPPQTIVVEMARENQ TTGKGKNNS | 780 |
| WP_003733029 | 707 | SFKSIIEKEQVST-TDKD-LOSIVAELAGSPAIKKGILQSLKIVDELVSVMG-YPPQTIVVEMARENQ TTNKGKNNS | 780 |
| WP_003739838 | 707 | SFKSIIEKEQVST-TDKD-LQSIVADLAGSPAIKKGILQSLKIVDELVSIMG-YPPQTIVVEMARENQ TTVKGKNNS | 780 |
| WP_014601172 | 707 | SFKSIIEKEQVST-TDKD-LOSIVADLAGSPAIKKGILQSLKIVDELVSIMG-YPPQTIVVEMARENQ TTGKGKNNS | 780 |
| WP_023548323 | 707 | SFKSIIEKEQVST-ADKD-LOSIVADLAGSPAIKKGILQSLKVVEELVSVMG-YPPQTIVVEMARENQ TTNKGKNNS | 780 |
| WP_031665337 | 707 | SFKSIIEKEQVST-TDKD-LQSIVAELAGSPAIKKGILQSLKIVDELVSIMG-YPPQTIVVEMARENQ TTGKGKNNS | 780 |
| WP_031669209 | 707 | SFKSIIEKEQVST-ADKD-LOSIVADLAGSPAIKKGILQSLKIVDELVSVMG-YPPQTIVVEMARENQ TTNKGKNNS | 780 |
| WP_033920898 | 707 | SFKSIIEKEQVST-ADKD-LQSIVADLAGSPAIKKGILQSLKVVEELVSVMG-YPPQTIVVEMARENQ TTNKGKNNS | 780 |
| AKI42028 | 710 | SFKSIIEKEQVST-TDKD-LOSIVADLAGSPAIKKGILQSLKIVDELVSIMG-YPPQTIVVEMARENQ TTGKGKNNS | 783 |
| AKI50529 | 710 | SFKSIIEKEQVST-ADKD-LOSIVADLAGSPAIKKGILQSLKVVEELVSVMG-YPPQTIVVEMARENQ TTNKGKNNS | 783 |
| EFR83390 | 155 | SFKSIIEKEQVST-TDKD-LOSIVADLAGSPAIKKGILQSLKIVDELVSIMG-YPPQTIVVEMARENQ TTVKGKNNS | 228 |
| WP_046323366 | 707 | SFKSIIEKEQVST-ADKD-IQSIVADLAGSPAIKKGILQSLKIVDELVGIMG-YPPQTIVVEMARENQ TTGKGKNNS | 780 |
| AKE81011 | 719 | TFKEDIQKAQVSG-QGDS-LHEHIANLAGSPAIKKGILQTVKVVDELVKVMGrHKPENIVIEMARENQ TTOKGQKNS | 793 |
| CUO82355 | 710 | GYAQMIEEASSCPKDGKF-TYEEVAKLAGSPALKRGIWQSLQIVEEITKVMK-CRPKYIYIEFERSEE -----KERT | 780 |
| WP_033162887 | 713 | GYKQIIEESNMQDIEGPF-KYDEVKKLAGSPAIKRGIWQALLVVREITKFMK-HEPSHIYIEFAREEQ -----KVRK | 783 |
| AGZ01981 | 736 | TFKEDIQKAQVSG-QGDS-LHEHIANLAGSPAIKKGILQTVKVVDELVKVMGrHKPENIVIEMARENQ TTQKGQKNS | 810 |
| AKA60242 | 703 | TFKEDIQKAQVSG-QGDS-LHEHIANLAGSPAIKKGILQTVKVVDELVKVMGrHKPENIVIEMARENQ TTOKGQKNS | 777 |
| AKS40380 | 703 | TFKEDIQKAQVSG-QGDS-LHEHIANLAGSPAIKKGILQTVKVVDELVKVMGrHKPENIVIEMARENQ TTQKGQKNS | 777 |
| 4UN5_B | 707 | TFKEDIQKAQVSG-QGDS-LHEHIANLAGSPAIKKGILQTVKVVDELVKVMGrHKPENIVIEMARENQ TTQKGQKNS | 781 |
| WP_010922251 | 778 | 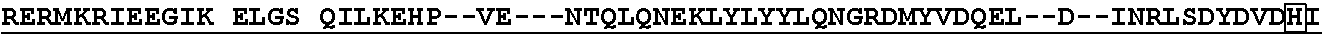 | 841 |
| WP_039695303 | 781 | QQRLKKLONSLK PSYI E----DK--VE---NSHLONDQLFLYYIQNGKDMYTGDEL--D--IDHLSDYDIDHI | 851 |
| WP_045635197 | 776 | QQRYKRIEDSLK ILAS NILKENP--TD---NNQLQNDRLFLYYLONGKDMYTGEAL--D--INQLSSYDIDHI | 843 |
| 5AXW_A | 488 | KDAQKMINEMQK QTNE EIIRTTGk--E---NAKYLIEKIKLHDMQEGKCLYSLEAIplEd1LNNPFNYEVDHI | 561 |
| WP_009880683 | 462 | RERMKRIEEGIK ELGS DILKEYP--VE---NTQLQNEKLYLYYLONGRDMYVDQEL--D--INRLSDYDVDHI | 525 |
| WP_010922251 | 778 | RERMKRIEEGIK ELGS QILKEHP--VE---NTQLQNEKLYLYYLQNGRDMYVDQEL--D--INRLSDYDVDHI | 841 |
| WP_011054416 | 778 | RERMKRIEEGIK ELGS DILKEYP--VE---NTQLQNEKLYLYYLONGRDMYVDQEL--D--INRLSDYDVDHI | 841 |
| WP_011284745 | 778 | RERMKRIEEGIK ELGS QILKEHP--VE---NTQLQNEKLYLYYLONGRDMYVDQEL--D--INRLSDYDVDHI | 841 |
| WP_011285506 | 778 | RERMKRIEEGIK ELGS QILKEHP--VE---NTQLQNEKLYLYYLONGRDMYVDQEL--D--INRLSDYDVDHI | 841 |
| WP_011527619 | 778 | RERMKRIEEGIK ELGS QILKEHP--VE---NTQLQNEKLYLYYLONGRDMYVDQEL--D--INRLSDYDVDHI | 841 |
| WP_012560673 | 778 | RERMKRIEEGIK ELGS DILKEYP--VE---TTQLQNEKLYLYYLONGRDMYVDQEL--D--INRLSDYDVDHI | 841 |
| WP_014407541 | 777 | RERMKRIEEGIK ELGS QILKEHP--VE---NTQLQNEKLYLYYLONGRDMYVDQEL--D--INRLSDYDVDHI | 840 |
| WP_020905136 | 778 | RERMKRIEEGIK ELGS QILKEHP--VE---NTQLQNEKLYLYYLONGRDMYVDQEL--D--INRLSDYDVDHI | 841 |
| WP_023080005 | 777 | RERMKRIEEGIK ELGS QILKEHP--VE---NTQLQNEKLYLYYLONGRDMYVDQEL--D--INRLSDYDVDHI | 840 |
| WP_023610282 | 777 | RERMKRIEEGIK ELGS QILKEHP--VE---NTQLQNEKLYLYYLQNGRDMYVDQEL--D--INRLSDYDVDHI | 840 |
| WP_030125963 | 778 | RERMKRIEEGIK ELGS DILKEYP--VE---NTQLQNEKLYLYYLONGRDMYVDQEL--D--INRLSDYDVDHI | 841 |
| WP_030126706 | 778 | RERMKRIEEGIK ELGS DILKEYP--VE---NTQLQNEKLYLYYLONGRDMYVDQEL--D--INRLSDYDVDHI | 841 |
| WP_031488318 | 778 | RERMKRIEEGIK ELGS DILKEYP--VE---NTQLQNEKLYLYYLONGRDMYVDQEL--D--INRLSDYDVDHI | 841 |
| WP_032460140 | 778 | RERMKRIEEGIK ELGS DILKEYP--VE---NTQLQNEKLYLYYLQNGRDMYVDQEL--D--INRLSDYDVDHI | 841 |
| WP_032461047 | 778 | RERMKRIEEGIK ELGS DILKEYP--VE---NTQLQNEKLYLYYLONGRDMYVDQEL--D--INRLSDYDVDHI | 841 |
| WP_032462016 | 778 | RERMKRIEEGIK ELGS QILKEHP--VE---NTQLQNEKLYLYYLONGRDMYVDQEL--D--INRLSDYDVDHI | 841 |
| WP_032462936 | 778 | RERMKRIEEGIK ELGS DILKEYP--VE---NTQLQNEKLYLYYLQNGRDMYVDQEL--D--INRLSDYDVDHI | 841 |
| WP_032464890 | 778 | RERMKRIEEGIK ELGS QILKEHP--VE---NTQLQNEKLYLYYLONGRDMYVDQEL--D--INRLSDYDVDHI | 841 |
| WP_033888930 | 603 | RERMKRIEEGIK ELGS QILKEHP--VE---NTQLQNEKLYLYYLONGRDMYVDQEL--D--INRLSDYDVDHI | 666 |
| WP_038431314 | 778 | RERMKRIEEGIK ELGS DILKEYP--VE---NTQLQNEKLYLYYLONGRDMYVDQEL--D--INRLSDYDVDHI | 841 |
| WP_038432938 | 777 | RERMKRIEEGIK ELGS QILKEHP--VE---NTQLQNEKLYLYYLONGRDMYVDQEL--D--INRLSDYDVDHI | 840 |
| WP_038434062 | 778 | RERMKRIEEGIK ELGS DILKEYP--VE---NTQLQNEKLYLYYLONGRDMYVDQEL--D--INRLSDYDVDHI | 841 |
| BAQ51233 | 689 | RERMKRIEEGIK ELGS QILKEHP--VE---NTQLQNEKLYLYYLONGRDMYVDQEL--D--INRLSDYDVDHI | 752 |
| KGE60162 | 1 | ------------ ---- -------------------------------------QEL--D--INRLSGYDVDHI | 16 |
| KGE60856 | | ------------ ---- --------------------------------------------------------- | |
| WP_002989955 | 778 | RERMKRIEEGIK ELGS QILKEHP--VE---NTQLQNEKLYLYYLONGRDMYVDQEL--D--INRLSDYDVDHI | 841 |
| WP_003030002 | 778 | QQRLKLLQDSLK PVNI K-----N--VE---NQQLQNDRLFLYYIQNGKDMYTGETL--D--INNLSQYDIDHI | 840 |
| WP_003065552 | 781 | QQRLKKLONSLK PSYI E----DK--VE---NSHLONDQLFLYYIQNGKDMYTGDEL--D--IDHLSDYDIDHI | 851 |
| WP_001040076 | 776 | RQRLTTLRESLA NLKS EKKPKYV--KDqveNHHLSDDRLFLYYLONGKDMYTDDEL--D--IDNLSQYDIDHI | 846 |
| WP_001040078 | 779 | RQRYKLLDDGVK NLAS NILKEYP--TD---NQALQNERLFLYYLONGRDMYTGEAL--D--IDNLSQYDIDHI | 846 |
| WP_001040080 | 779 | RQRYKLLDDGVK NLAS NILKEYP--TD---NQALQNERLFLYYLQNGRDMYTGEAL--D--IDNLSQYDIDHI | 846 |
| WP_001040081 | 779 | RQRYKLLDDGVK NLAS NILKEYP--TD---NQALQNERLFLYYLONGRDMYTGEAL--D--IDNLSQYDIDHI | 846 |
| WP_001040083 | 779 | RQRYKLLDDGVK NLAS NILKEYP--TD---NQALQNERLFLYYLONGRDMYTGEAL--D--IDNLSQYDIDHI | 846 |
| WP_001040085 | 779 | RQRYKLLDDGVK NLAS NILKEYP--TD---NQALONERLFLYYLONGRDMYTGEAL--D--IDDLSQYDIDHI | 846 |
| WP_001040087 | 779 | RQRYKLLDDGVK NLAS NILKEYP--TD---NQALQNERLFLYYLONGRDMYTGEAL--D--IDNLSQYDIDHI | 846 |
| WP_001040088 | 779 | RQRYKLLDDGVK NLAS NILKEYP--TD---NQALQNERLFLYYLONGRDMYTGEAL--D--IDNLSQYDIDHI | 846 |
| WP_001040089 | 779 | RQRYKLLDDGVK NLAS NILKEYP--TD---NQALQNERLFLYYLONGRDMYTGEAL--D--IDNLSQYDIDHI | 846 |
| WP_001040090 | 779 | RQRYKLLDDGVK NLAS NILKEYP--TD---NQALQNERLFLYYLONGRDMYTGEAL--D--IDNLSQYDIDHI | 846 |
| WP_001040091 | 779 | RQRYKLLDDGVK NLAS NILKEYP--TD---NQALQNERLFLYYLONGRDMYTGEAL--D--IDNLSQYDIDHI | 846 |
| WP_001040092 | 779 | RQRYKLLEDGVK NLAS DILKEYP--TD---NQALONERLFLYYLONGRDMYTGEAL--D--IDNLSQYDIDHI | 846 |
| WP_001040094 | 776 | RQRLTTLRESLA NLKS EKKPKYV--KDqveNHHLSDDRLFLYYLONGKDMYTDDEL--D--IDNLSQYDIDHI | 846 |
| WP_001040095 | 776 | RQRLTTLRESLA NLKS EKKPKYV--KDqveNHHLSDDRLFLYYLONGKDMYTDDEL--D--IDNLSQYDIDHI | 846 |
| WP_001040096 | 776 | RORLTTLRESLA NLKS EKKPKYV--KDqveNHHLSDDRLFLYYLONGKDMYTDDEL--D--IDNLSQYDIDHI | 846 |
| WP_001040097 | 776 | RORLTTLRESLA NLKS EKKPKYV--KDqveNHHLSDDRLFLYYLONGKDMYTDDEL--D--IDNLSQYDIDHI | 846 |
| WP_001040098 | 776 | RQRLTTLRESLA NLKS EKKPKYV--KDqveNHHLSDDRLFLYYLONGKDMYTDDEL--D--IDNLSQYDIDHI | 846 |
| WP_001040099 | 776 | RORLTTLRESLA NLKS EKKPKYV--KDqveNHHLSDDRLFLYYLONGKDMYTDDEL--D--IDNLSQYDIDHI | 846 |
| WP_001040100 | 776 | RORLTTLRESLA NLKS EKKPKYV--KDqveNHHLSDDRLFLYYLONGKDMYTDDEL--D--IDNLSQYDIDHI | 846 |
| WP_001040104 | 779 | RQRYKLLDDGVK NLAS NILKEYP--TD---NQALONERLFLYYLONGRDMYTGEAL--D--IDNLSQYDIDHI | 846 |
| WP_001040105 | 779 | RQRYKLLDDGVK NLAS NILKEYP--TD---NQALQNERLFLYYLONGRDMYTGEAL--D--IDNLSQYDIDHI | 846 |
| WP_001040106 | 779 | RORYKLLEEGVK NLAS NILKEYP--TD---NQALQNERLFLYYLONGRDMYTGEAL--D--IDNLSQYDIDHI | 846 |
| WP_001040107 | 779 | RQRYKLLEEGVK NLAS NILKEYP--TD---NQALQNERLFLYYLONGRDMYTGEAL--D--IDNLSQYDIDHI | 846 |
| WP_001040108 | 779 | RORYKLLEEGVK NLAS NILKEYP--TD---NQALQNERLFLYYLONGRDMYTGETL--D--IDNLSQYDIDHI | 846 |
| WP_001040109 | 779 | RORYKLLEEGVK NLAS NILKEYP--TD---NQALQNERLFLYYLONGRDMYTGEAL--D--IDNLSQYDIDHI | 846 |
| WP_001040110 | 779 | RORYKLLEEGVK NLAS NILKEYP--TD---NQALQNERLFLYYLONGRDMYTGEAL--D--IDNLSQYDIDHI | 846 |
| WP_015058523 | 779 | RQRYKLLEDGVK NLAS DILKEYP--TD---NQALQNERLFLYYLONGRDMYTGEAL--D--IDNLSQYDIDHI | 846 |
| WP_017643650 | 776 | RQRLTTLRESLA NLKS EKKPKYV--KDqveNHHLSDDRLFLYYLONGKDMYTDDEL--D--IDNLSQYDIDLI | 846 |
| WP_017647151 | 779 | RQRYKLLDDGVK NLAS NILKEYP--TD---NQALONERLFLYYLONGRDMYTGKAL--D--IDNLSQYDIDHI | 846 |
| WP_017648376 | 779 | RQRYKLLDDGVK NLAS NILKEYP--TD---NQALQNERLFLYYLQNGRDMYTGKAL--D--IDNLSQYDIDHI | 846 |
| WP_017649527 | 779 | RORYKLLDDGVK NLAS NILKEYP--TD---NQALONERLFLYYLQNGRDMYTGEAL--D--IDNLSQYDIDHI | 846 |
| WP_017771611 | 779 | RORYKLLEEGVK NLAS NILKEYP--TD---NQALONERLFLYYLONGRDMYTGEAL--D--IDNLSQYDIDHI | 846 |
| WP_017771984 | 779 | RQRYKLLDDGVK NLAS NILKEYP--TD---NQALQNERLFLYYLONGRDMYTGEAL--D--IDNLSQYDIDHI | 846 |
| CFQ25032 | 779 | RQRYKLLDDGVK NLAS NILKEYP--TD---NQALQNERLFLYYLONGRDMYTGEAL--D--IDNLSQYDIDHI | 846 |
| CFV16040 | 779 | RQRYKLLDDGVK NLAS NILKEYP--TD---NQALQNERLFLYYLONGRDMYTGEAL--D--IDNLSQYDIDHI | 846 |
| KLJ37842 | 779 | RORYKLLDDGVK NLAS NILKEYP--TD---NQALQNERLFLYYLONGRDMYTGEAL--D--IDNLSQYDIDHI | 846 |
| KLJ72361 | 779 | RQRYKLLDDGVK NLAS NILKEYP--TD---NQALQNERLFLYYLONGRDMYTGEAL--D--IDNLSQYDIDHI | 846 |
| KLL20707 | 793 | RORYKLLDDGVK NLAS NILKEYP--TD---NQALQNERLFLYYLONGRDMYTGEAL--D--IDNLSQYDIDHI | 860 |
| KLL42645 | 779 | RORYKLLEEGVK NLAS NILKEYP--TD---NQALONERLFLYYLONGRDMYTGEAL--D--IDNLSQYDIDHI | 846 |
| WP_047207273 | 779 | RQRYKLLDDGVK NLAS NILKEYP--TD---NQALQNERLFLYYLONGRDMYTGEAL--D--IDNLSQYDIDHI | 846 |
| WP_047209694 | 776 | RQRLTTLRESLA NLKS EKKPKYV--KDqveNHHLSDDRLFLYYLQNGKDMYTDDEL--D--IDNLSQYDIDHI | 846 |
| WP_050198062 | 779 | RQRYKLLDDGVK NLAS NILKEYP--TD---NQALQNERLFLYYLONGRDMYTGEAL--D--IDNLSQYDIDHI | 846 |
| WP_050201642 | 779 | RQRYKLLDDGVK NLAS NILKEYP--TD---NQALQNERLFLYYLONGRDMYTGEAL--D--IDNLSQYDIDHI | 846 |
| WP_050204027 | 779 | RQRYKLLEEGVK NLAS NILKEYP--TD---NQALQNERLFLYYLONGRDMYTGEAL--D--IDNLSQYDIDHI | 846 |
| WP_050881965 | 779 | RORYKLLDDGVK NLAS NILKEYP--TD---NQALQNERLFLYYLONGRDMYTGEAL--D--IDNLSQYDIDHI | 846 |
| WP_050886065 | 779 | RORYKLLDDGVK NLAS NILKEYP--TD---NQALQNERLFLYYLONGRDMYTGEAL--D--IDNLSQYDIDHI | 846 |
| AHN30376 | 779 | RQRYKLLEDGVK NLAS DILKEYP--TD---NQALQNERLFLYYLONGRDMYTGEAL--D--IDSLSQYDIDHI | 846 |
| EAO78426 | 779 | RQRYKLLDDGVK NLAS NILKEYP--TD---NQALQNERLFLYYLONGRDMYTGEAL--D--IDNLSQYDIDHI | 846 |
| CCW42055 | 779 | RQRYKLLDDGVR NLAS NILKEYP--TD---NQALQNERLFLYYLONGRDMYTEKAL--D--IDNLSQYDIDHI | 846 |
| WP_003041502 | 778 | QQRLKLLQDSLK PVNI K-----N--VE---NQQLQNDRLFLYYIQNGKDMYTGETL--D--INNLSQYDIDHI | 840 |
| WP_037593752 | 779 | QQRLKLLQDSLK PVNI K-----N--VE---NQQLQNDRLFLYYIQNGKDMYTGETL--D--INNLSQYDIDHI | 841 |
| WP_049516684 | 779 | QQRLKLLQDSLK PVNI K-----N--VE---NQQLQNDRLFLYYIQNGKDMYTGETL--D--INNLSQYDIDHI | 841 |
| GAD46167 | 778 | QQRLKLLQDSLK PVNI K-----N--VE---NQQLQNDRLFLYYIQNGKDMYTGETL--D--INNLSQYDIDHI | 840 |
| WP_018363470 | 779 | QQRLKKLQNSLK PSYI E----DK--VE---NSHLONDQLFLYYIQNGKDMYTGDEL--D--IDHLSDYDIDHI | 849 |
| WP_003043819 | 787 | RERKKRIEEGIK ELES QILKENP--VE---NTQLQNEKLYLYYLONGRDMYVDQEL--D--INRLSDYDVDHI | 850 |
| WP_006269658 | 778 | QQRLKLLQDSLK PVNI K-----N--VE---NQQLQNDRLFLYYIQNGKDMYTGETL--D--INNLSQYDIDHI | 840 |
| WP_048800889 | 778 | QQRLKLLQDSLT PVSI K-----N--VE---NQQLQNDRLFLYYIQNGKDMYTGEEL--D--IHHLSDYDIDHI | 840 |
| WP_012767106 | 777 | RERMKRIEEGIK ELGS QILKEHP--VE---NTQLQNEKLYLYYLONGRDMYVDQEL--D--INRLSDYDVDHI | 840 |
| WP_014612333 | 777 | RERMKRIEEGIK ELGS QILKEHP--VE---NTQLQNEKLYLYYLONGRDMYVDQEL--D--INRLSDYDVDHI | 840 |
| WP_015017095 | 777 | RERMKRIEEGIK ELGS QILKEHP--VE---NTQLQNEKLYLYYLONGRDMYVDQEL--D--INRLSDYDVDHI | 840 |
| WP_015057649 | 777 | RERMKRIEEGIK ELGS QILKEHP--VE---NTQLQNEKLYLYYLONGRDMYVDQEL--D--INRLSDYDVDHI | 840 |
| WP_048327215 | 777 | RERMKRIEEGIK ELGS QILKEHP--VE---NTQLQNEKLYLYYLONGRDMYVDQEL--D--INRLSDYDVDHI | 840 |
| WP_049519324 | 777 | RERMKRIEEGIK ELGS QILKEHP--VE---NTQLQNEKLYLYYLONGRDMYVDQEL--D--INRLSDYDVDHI | 840 |
| WP_012515931 | 777 | RORMRKLEETAK KLGS NILKEHP--VD---NSQLQNDKRYLYYLONGKDMYTGDDL--D--IDYLSSYDIDHI | 840 |
| WP_021320964 | 777 | RORMRKLEETAK KLGS NILKEHP--VD---NSQLQNDKRYLYYLONGKDMYTGDDL--D--IDYLSSYDIDHI | 840 |
| WP_037581760 | 777 | RQRMRKLEETAK KLGS NILKEHP--VD---NSQLQNDKRYLYYLONGKDMYTGDDL--D--IDYLSSYDIDHI | 840 |
| WP_004232481 | 778 | NORLKRLQDSLK PSYV D----SK--VE---NSHLONDRLFLYYIQNGKDMYTGEEL--D--IDHLSDYDIDHI | 848 |
| WP_009854540 | 779 | QQRLKKLQSSLK PSYI E----DK--VE---NSHLONDQLFLYYIQNGKDMYTGDEL--D--IDHLSDYDIDHI | 849 |
| WP_012962174 | 779 | QQRLKKLQDSLK PSYI E----GK--VE---NNHLQDDRLFLYYIQNGKDMYTGDEL--D--IDHLSDYDIDHI | 849 |
| WP_039695303 | 781 | QQRLKKLQNSLK PSYI E----DK--VE---NSHLQNDQLFLYYIQNGKDMYTGDEL--D--IDHLSDYDIDHI | 851 |
| WP_014334983 | 778 | NQRLKRLQDSLK PSYV D----SK--VE---NSHLQNDRLFLYYIQNGKDMYTGEEL--D--IDRLSDYDIDHI | 848 |
| WP_003099269 | 778 | RQRLRKLEEVHK NTGS KILKEYN--VS---NTQLQSDRLYLYLLQDGKDMYTGKEL--D--YDNLSQYDIDHI | 841 |
| AHY15608 | 778 | RQRLRKLEEVHK NTGS KILKEYN--VS---NTQLQSDRLYLYLLQDGKDMYTGKEL--D--YDNLSQYDIDHI | 841 |
| AHY17476 | 778 | RQRLRKLEEVHK NTGS KILKEYN--VS---NTQLQSDRLYLYLLQDGKDMYTGKEL--D--YDNLSQYDIDHI | 841 |
| ESR09100 | | ------------ ---- --------------------------------------------------------- | |
| AGM98575 | 778 | RORLRKLEEVHK NTGS KILKEYN--VS---NTQLQSDRLYLYLLQDGKDMYTGKEL--D--YDNLSQYDIDHI | 841 |
| ALF27331 | 778 | QQRLKGLTDSIK EFGS QILKEHP--VE---NSQLQNDRLFLYYLONGRDMYTGEEL--D--IDYLSQYDIDHI | 841 |
| WP_018372492 | 791 | AQRLKKIEDGIK -LGS DLLKONP--IQd--NKDLQKEKLFLYYMONGIDLYTGQPLncD--PDSLAFYDVDHI | 857 |
| WP_045618028 | 777 | QQRYKRIEDALK NLAH NILKEHP--TD---NIQLQNDRLFLYYLONGKDMYTGKSL--D--INQLSSCDIDHI | 844 |
| WP_045635197 | 776 | QQRYKRIEDSLK ILAS NILKENP--TD---NNQLQNDRLFLYYLONGKDMYTGEAL--D--INQLSSYDIDHI | 843 |
| WP_002263549 | 778 | QQRLKGLTDSIK EFGS QILKEHP--VE---NSQLQNDRLFLYYLONGRDMYTGEEL--D--IDYLSQYDIDHI | 841 |
| WP_002263887 | 778 | QQRLKGLTDSIK EFGS QILKEHP--VE---NSQLQNDRLFLYYLONGRDMYTGEEL--D--IDYLSQYDIDHI | 841 |
| WP_002264920 | 778 | QQRLKGLTDSIK EFGS QILKEHP--VE---NSQLQNDRLFLYYLONGRDMYTGEEL--D--IDYLSQYDIDHI | 841 |
| WP_002269043 | 778 | QQRLKGLTDSIK EFGS QILKEHP--VE---NSQLQNDRLFLYYLONGRDMYTGEEL--D--IDYLSQYDIDHI | 841 |
| WP_002269448 | 778 | QQRLKGLTDSIK EFGS QILKEHP--VE---NSQLQNDRLFLYYLONGRDMYTGEEL--D--IDYLSQYDIDHI | 841 |
| WP_002271977 | 778 | QQRLKGLTDSIK EFGS QILKEHP--VE---NSQLQNDRLFLYYLONGRDMYTGEEL--D--IDYLSQYDIDHI | 841 |
| WP_002272766 | 778 | QQRLKGLTDSIK EFGS QILKEHP--VE---NSQLQNDRLFLYYLONGRDMYTGEEL--D--IDYLSQYDIDHI | 841 |
| WP_002273241 | 778 | QQRLKGLTDSIK EFGS QILKEHP--VE---NSQLQNDRLFLYYLONGRDMYTGEEL--D--IDYLSQYDIDHI | 841 |
| WP_002275430 | 778 | QQRLKGLTDSIK EFGS QILKEHP--VE---NSQLQNDRLFLYYLONGRDMYTGEEL--D--IDYLSQYDIDHI | 841 |
| WP_002276448 | 778 | QQRLKGLTDSIK EFGS QILKEHP--VE---NSQLQNDRLFLYYLONGRDMYTGEEL--D--IDYLSQYDIDHI | 841 |
| WP_002277050 | 779 | QQRYKRLKEAIK DLNH KILKEHP--TD---NQALONNRLFLYYLONGRDMYTGESL--D--INRLSDYDIDHV | 846 |
| WP_002277364 | 778 | QQRLKGLTDSIK EFGS QILKEHP--VE---HSQLQNDRLFLYYLONGRDMYTGEEL--D--IDYLSQYDIDHI | 841 |
| WP_002279025 | 778 | QQRLKGLTDSIK EFGS QILKEHP--VE---NSQLQNDRLFLYYLONGRDMYTGEEL--D--IDYLSQYDIDHI | 841 |
| WP_002279859 | 778 | QQRLKGLTDSIK EFGS QILKEHP--VE---NSQLQNDRLFLYYLONGRDMYTGEEL--D--IDYLSQYDIDHI | 841 |
| WP_002280230 | 778 | QQRLKGLTDSIK EFGS QILKEHP--VE---NSQLQNDRLFLYYLONGRDMYTGEEL--D--IDYLSQYDIDHI | 841 |
| WP_002281696 | 778 | QQRLKGLTDSIK EFGS QILKEHP--VE---NSQLQNDRLFLYYLONGRDMYTGEEL--D--IDYLSQYDIDHI | 841 |
| WP_002282247 | 779 | QQRYKRLKEAIK DLNH KILKEHP--TD---NQALONNRLFLYYLONGRDMYTGESL--D--INRLSDYDIDHV | 846 |
| WP_002282906 | 778 | QQRLKGLTDSIK EFGS QILKEHP--VE---NSQLQNDRLFLYYLQNGRDMYTGEEL--D--IDYLSQYDIDHI | 841 |
| WP_002283846 | 778 | QQRLKGLTDSIK EFGS QILKEHP--VE---NSQLQNDRLFLYYLONGRDMYTGEEL--D--IDYLSQYDIDHI | 841 |
| WP_002287255 | 778 | QQRLKGLTDSIK EFGS QILKEHP--VE---HSQLQNDRLFLYYLONGRDMYTGEEL--D--IDYLSQYDIDHI | 841 |
| WP_002288990 | 778 | QQRLKGLTDSIK EFGS QILKEHP--VE---NSQLQNDRLFLYYLONGRDMYTGEEL--D--IDYLSQYDIDHI | 841 |
| WP_002289641 | 778 | QQRLKGLTDSIK EFGS QILKEHP--VE---NSQLONDRLFLYYLONGRDMYTGEEL--D--IDYLSQYDIDHI | 841 |
| WP_002290427 | 778 | QQRLKGLTDSIK EFGS QILKEHP--VE---NSQLONDRLFLYYLONGRDMYTGEEL--D--IDYLSQYDIDHI | 841 |
| WP_002295753 | 778 | QQRLKGLTDSIK EFGS QILKEHP--VE---NSQLQNDRLFLYYLONGRDMYTGEEL--D--IDYLSQYDIDHI | 841 |
| WP_002296423 | 778 | QQRLKGLTDSIK EFGS QILKEHP--VE---NSQLQNDRLFLYYLONGRDMYTGEEL--D--IDYLSQYDIDHI | 841 |
| WP_002304487 | 788 | QKRYKRLEEAIK DINH KILKEHP--TD---NQALQNDRLFLYYLONGRDMYTEDPL--D--INRLSDYDIDHI | 855 |
| WP_002305844 | 778 | QQRLKGLTDSIK EFGS QILKEHP--VE---NSQLQNDRLFLYYLONGRDMYTGEEL--D--IDYLSQYDIDHI | 841 |
| WP_002307203 | 778 | QQRLKGLTDSIK EFGS QILKEHP--VE---NSQLQNDRLFLYYLONGRDMYTGEEL--D--IDYLSQYDIDHI | 841 |
| WP_002310390 | 778 | QQRLKGLTDSIK EFGS QILKEHP--VE---NSQLQNDRLFLYYLONGRDMYTGEEL--D--IDYLSQYDIDHI | 841 |
| WP_002352408 | 778 | QQRLKGLTDSIK EFGS QILKEHP--VE---NSQLQNDRLFLYYLONGRDMYTGEEL--D--IDYLSQYDIDHI | 841 |
| WP_012997688 | 778 | QQRLKGLTDSIK EFGS QILKEHP--VK---HSQLQNDRLFLYYLONGRDMYTGEEL--D--IDYLSQYDIDHI | 841 |
| WP_014677909 | 778 | QQRLKGLTDSIK EFGS QILKEHP--VE---NSQLQNDRLFLYYLONGRDMYTGEEL--D--IDYLSQYDIDHI | 841 |
| WP_019312892 | 778 | QQRLKGLTDSIK EFGS QILKEHP--VE---NSQLQNDRLFLYYLONGRDMYTGEEL--D--IDYLSQYDIDHI | 841 |
| WP_019313659 | 778 | QQRLKGLTDSIK EFGS QILKEHP--VE---NSQLQNDRLFLYYLQNGRDMYTGEEL--D--IDYLSQYDIDHI | 841 |
| WP_019314093 | 778 | QQRLKGLTDSIK EFGS QILKEHP--VE---NSQLQNDRLFLYYLONGRDMYTGEEL--D--IDYLSQYDIDHI | 841 |
| WP_019315370 | 778 | QQRLKGLTDSIK EFGS QILKEHP--VE---NSQLQNDRLFLYYLONGRDMYTGEEL--D--IDYLSQYDIDHI | 841 |
| WP_019803776 | 778 | QQRLKGLTDSIK EFGS QILKEHP--VE---NSQLQNDRLFLYYLONGRDMYTGEEL--D--IDYLSQYDIDHI | 841 |
| WP_019805234 | 778 | QQRLKGLTDSIK EFGS QILKEHP--VE---NSQLQNDRLFLYYLONGRDMYTGEEL--D--IDYLSQYDIDHI | 841 |
| WP_024783594 | 778 | QQRLKGLTDSIK EFGS QILKEHP--VE---NSQLONDRLFLYYLONGRDMYTGEEL--D--IDYLSQYDIDHI | 841 |
| WP_024784288 | 779 | QQRYKRLKEAIK DLNH KILKEHP--TD---NQALONNRLFLYYLONGRDMYTGESL--D--INRLSDYDIDHV | 846 |
| WP_024784666 | 778 | QQRLKGLTDSIK EFGS QILKEHP--VE---HSQLQNDRLFLYYLONGRDMYTGEEL--D--IDYLSQYDIDHI | 841 |
| WP_024784894 | 778 | QQRLKGLTDSIK EFGS QILKEHP--VE---NSQLQNDRLFLYYLONGRDMYTGEEL--D--IDYLSQYDIDHI | 841 |
| WP_024786433 | 779 | QQRYKRLKEAIK DLNH KILKEHP--TD---NQALONNRLFLYYLONGRDMYTGESL--D--INRLSDYDIDHV | 846 |
| WP_049473442 | 778 | QQRLKGLTDSIK EFGS QILKEHP--VE---NSQLQNDRLFLYYLONGRDMYTGEEL--D--IDYLSQYDIDHI | 841 |
| WP_049474547 | 778 | QQRLKGLTDSIK EFGS QILKEHP--VE---NSQLQNDRLFLYYLONGRDMYTGEEL--D--IDYLSQYDIDHI | 841 |
| EMC03581 | 771 | QQRLKGLTDSIK EFGS QILKEHP--VE---HSQLQNDRLFLYYLONGRDMYTGEEL--D--IDYLSQYDIDHI | 834 |
| WP_000428612 | 779 | QQRYKRIEDSLK ILAS KILKEHP--TD---NIQLQNDRLFLYYLONGRDMYTGKPL--D--INQLSSYDIDHI | 846 |
| WP_000428613 | 777 | QQRYKRIEDALK NLAS NILKEHP--TN---NIQLQNDRLFLYYLONGRDMYTGKPL--D--INQLSSYDIDHI | 844 |
| WP_049523028 | 776 | QQRLKTLSDAIS ELG- NILKEHP--TD---NIQLQNDRLFLYYLQNGKDMYTGEAL--D--INQLSNYDIDHI | 839 |
| WP_003107102 | 747 | RERLRKLEEVHK NIGS KILKEHE--IS---NAQLQSDRVYLYLLQDGKDMYTGKDL--D--FDRLSQYDIDHI | 810 |
| WP_054279288 | 779 | RERMKRVQEVLK KLGS QLLKEHP--VE---NFQLQNERLYLYYLONGKDMYTGEEL--S--ISNLSHYDIDHI | 842 |
| WP_049531101 | 777 | QQRYKRIEDSLK ILAS NILKEHP--TD---NIQLQNDRLFLYYLONGKDMYTGNPL--D--INHLSSYDIDHI | 844 |
| WP_049538452 | 777 | QQRYKRIENSLK ILAS KILKEHP--TD---NNQLQNDRLFLYYLONGKDMYTGEAL--D--INQLSSCDIDHI | 844 |
| WP_049549711 | 777 | QQRYKRIEDSLK ILAS NILKENP--TD---NNQLQNDRLFLYYLONGKDMYTGEAL--D--INQLSSYDIDHI | 844 |
| WP_007896501 | 781 | RQRLKKIKEVHK KTGS RILEDNSerIT---NLTLQDNRLYLYLLQDGKDMYTGQDL--D--INNLSQYDIDHI | 846 |
| EFR44625 | 733 | RQRLKKIKEVHK KTGS RILEDNSerIT---NLTLQDNRLYLYLLQDGKDMYTGQDL--D--INNLSQYDIDHI | 798 |
| WP_002897477 | 776 | QQRYKRIEDALK NLAP NILKENP--TD---NIQLKNDRLFLYYLONGKDMYTGKPL--D--INQLSSYDIDHI | 843 |
| WP_002906454 | 776 | QQRYKRIEDALK NLAP NILKENP--TD---NIQLQNDRLFLYYLONGKDMYTGKAI--D--INQLSNYDIDHI | 843 |
| WP_009729476 | 777 | QQRYKRIEDSLK ILAS KILKEHP--TD---NIQLQNDRLFLYYLONGKDMYTGEAL--D--INQLSSCDIDHI | 844 |
| COR24647 | 780 | QQRLGSLTKAIQ DFGS DILKRYP--VE---NNQLQNDQLYLYYLONGKDMYTGDTL--D--IHNLSQYDIDHI | 843 |
| WP_000066813 | 779 | QQRYKRIEDSLK NLAS NILKENP--TD---NIQLQNDRLFLYYLONGRDMYTGKPL--E--INQLSNYDIDHI | 846 |
| WP_009754323 | 777 | QQRYKRIEDALK NLAP TISKENP--TD---NIQLQNDRLFLYYLONGKDMYTGEAL--D--INQLSSYDIDHI | 844 |
| WP_044674937 | 776 | QQRYKKIENAIK NLNS KILKEYP--TN---NQALQNDRLFLYYLONGKDMYTDEEL--D--IDQLSQYDIDHI | 843 |
| WP_044676715 | 778 | QQRYKKIENAIK NLNS KILKEYP--TN---NQALQNDRLFLYYLONGKDMYTDEEL--D--IDQLSQYDIDHI | 845 |
| WP_044680361 | 778 | QQRYKKIENAIK NLNS KILKEYP--TN---NQALQNDRLFLYYLONGKDMYTDEEL--D--IDOLSQYDIDHI | 845 |
| WP_044681799 | 776 | QQRYKKIENAIK NLNS KILKEYP--TN---NQALQNDRLFLYYLONGKDMYTDEEL--D--IDOLSQYDIDHI | 843 |
| WP_049533112 | 778 | QQRLKLLQDSLK PVNI K-----N--VE---NQQLQNDRLFLYYIQNGKDMYTGETL--D--INNLSQYDIDHI | 840 |
| WP_029090905 | 753 | TPRDKFIEKAYA ETDT EHLKELK---Qr--SKQLSSQRLFLYFIQNGKCMYSGEHL--D--IERLDSYEVDHI | 823 |
| WP_006506696 | 777 | ESKIKKLENVYK DEQT SVLEELKg-FDn--TKKISSDSLFLYFTQLGKCMYSGKKL--D--IDSLDKYQIDHI | 849 |
| AIT42264 | 778 | RERMKRIEEGIK ELGS QILKEHP--VE---NTQLQNEKLYLYYLONGRDMYVDQEL--D--INRLSDYDVDHI | 841 |
| WP_034440723 | 785 | KARLKKIQEGLE NLDS HVEKQAL---D---EEMLKSPKYYLYCLONGKDIYTGKDL--D--IGQLQTYDIDHI | 848 |
| AKQ21048 | 778 | RERMKRIEEGIK ELGS QILKEHP--VE---NTQLQNEKLYLYYLONGRDMYVDQEL--D--INRLSDYDVDHI | 841 |
| WP_004636532 | 781 | KERLEKLTEAIK EFDG --VKVKD--LK---NENLRNDRLYLYYLONGRDMYTNEPL--D--INNLSKYDIDHI | 845 |
| WP_002364836 | 789 | IQRLKIVEKAMA EIGS NLLKEQP--TT---NEQLRDTRLFLYYMONGKDMYTGDEL--S--LHRLSHYDIDHI | 852 |
| WP_016631044 | 740 | IQRLKIVEKAMA EIGS NLLKEQP--TT---NEQLRDTRLFLYYMONGKDMYTGDEL--S--LHRLSHYDIDHI | 803 |
| EMS75795 | 525 | KPRLKALEEALK SFDS PLLKEQP--VD---NQALOKDRLYLYYLONGKDMYTGEAL--D--IDRLSEYDIDHI | 588 |
| WP_002373311 | 789 | IQRLKIVEKAMA EIGS NLLKEQP--TT---NEQLRDTRLFLYYMONGKDMYTGDEL--S--LHRLSHYDIDHI | 852 |
| WP_002378009 | 789 | IQRLKIVEKAMA EIGS NLLKEQP--TT---NEQLRDTRLFLYYMONGKDMYTGDEL--S--LHRLSHYDIDHI | 852 |
| WP_002407324 | 789 | IQRLKIVEKAMA EIGS NLLKEQP--TT---NEQLRDTRLFLYYMQNGKDMYTGDEL--S--LHRLSHYDIDHI | 852 |
| WP_002413717 | 789 | IQRLKIVEKAMA EIGS NLLKEQP--TT---NEQLRDTRLFLYYMONGKDMYTGDEL--S--LHRLSHYDIDHI | 852 |
| WP_010775580 | 791 | IQRLKIVEKAMA EIGS NLLKEQP--TT---NEQLRDTRLFLYYMONGKDMYTGDEL--S--LHRLSHYDIDHI | 854 |
| WP_010818269 | 789 | IQRLKIVEKAMA EIGS NLLKEQP--TT---NEQLRDTRLFLYYMONGKDMYTGDEL--S--LHRLSHYDIDHI | 852 |
| WP_010824395 | 789 | IQRLKIVEKAMA EIGS NLLKEQP--TT---NEQLRDTRLFLYYMONGKDMYTGDEL--S--LHRLSHYDIDHI | 852 |
| WP_016622645 | 789 | IQRLKIVEKAMA EIGS NLLKEQP--TT---NEQLRDTRLFLYYMONGKDMYTGDEL--S--LHRLSHYDIDHI | 852 |
| WP_033624816 | 789 | IQRLKIVEKAMA EIGS NLLKEQP--TT---NEQLRDTRLFLYYMONGKDMYTGDEL--S--LHRLSHYDIDHI | 852 |
| WP_033625576 | 789 | IQRLKIVEKAMA EIGS NLLKEQP--TT---NEQLRDTRLFLYYMONGKDMYTGDEL--S--LHRLSHYDIDHI | 852 |
| WP_033789179 | 789 | IQRLKIVEKAMA EIGS NLLKEQP--TT---NEQLRDTRLFLYYMONGKDMYTGDEL--S--LHRLSHYDIDHI | 852 |
| WP_002310644 | 789 | RPRLKALEESLK DFGS QLLKEYP--TD---NSSLOKDRLYLYYLONGRDMYTGAPL--D--IHRLSDYDIDHI | 852 |
| WP_002312694 | 790 | RPRLKALEESLK DFGS QLLKEYP--TD---NSSLOKDRLYLYYLONGRDMYTGAPL--D--IHRLSDYDIDHI | 853 |
| WP_002314015 | 790 | RPRLKALEESLK DFGS QLLKEYP--TD---NSSLOKDRLYLYYLONGRDMYTGAPL--D--IHRLSDYDIDHI | 853 |
| WP_002320716 | 790 | RPRLKALEESLK DFGS QLLKEYP--TD---NSSLOKDRLYLYYLONGRDMYTGAPL--D--IHRLSDYDIDHI | 853 |
| WP_002330729 | 789 | RPRLKALEESLK DFGS QLLKEYP--TD---NSSLOKDRLYLYYLONGRDMYTGAPL--D--IHRLSDYDIDHI | 852 |
| WP_002335161 | 790 | RPRLKALEESLK DFGS QLLKEYP--TD---NSSLOKDRLYLYYLONGRDMYTGAPL--D--IHRLSDYDIDHI | 853 |
| WP_002345439 | 790 | RPRLKALEESLK DFGS QLLKEYP--TD---NSSLOKDRLYLYYLONGRDMYTGAPL--D--IHRLSDYDIDHI | 853 |
| WP_034867970 | 781 | SPRLKALENGLK QIGS TLLKEQP--TD---NKALQKERLYLYYLONGRDMYTGEPL--E--IENLHQYEVDHI | 844 |
| WP_047937432 | 790 | RPRLKALEESLK DEGS QLLKEYP--TD---NSSLOKDRLYLYYLONGRDMYTGAPL--D--IHRLSDYDIDHI | 853 |
| WP_010720994 | 781 | KPRLKALENGLK QIGS TLLKEQP--TD---NKALQKERLYLYYLONGRDMYTGEPL--E--IENLHQYEVDHI | 844 |
| WP_010737004 | 781 | SPRLKALENGLK QIGS TLLKEQP--TD---NKALQKERLYLYYLONGRDMYTGEPL--E--IENLHQYEVDHI | 844 |
| WP_034700478 | 781 | KPRLKALENGLK QIGS TLLKEQP--TD---NKALQKERLYLYYLONGRDMYTGEPL--E--IENLHQYEVDHI | 844 |
| WP_007209003 | 782 | KPRLKGIENGLK EFSD SVLKGSS--ID---NKQLQNDRLYLYYLQNGKDMYTGHEL--D--IDHLSTYDIDHI | 845 |
| WP_023519017 | 775 | RPRLKALEEALK NIDS PLLKDYP--TD---NQALQKDRLYLYYLONGKDMYTGEPL--E--IHRLSEYDIDHI | 838 |
| WP_010770040 | 782 | NPRMKALEEAMR NLRS NLLKEYP--TD---NQALQNDRLYLYYLONGKDMYTGLDL--S--LHNLSSYDIDHI | 845 |
| WP_048604708 | 779 | RPRLKNLEKAID DLDS EILKKHP--VD---NKALQKDRLYLYYLONGKDMYTNEEL--D--IHKLSTYDIDHI | 842 |
| WP_010750235 | 784 | KPRLKSLEEALK NEDS QLLKERP--VD---NQSLOKDRLYLYYLONGKDMYTGESL--D--IDRLSEYDIDHI | 847 |
| AII16583 | 817 | RERMKRIEEGIK ELGS QILKEHP--VE---NTQLQNEKLYLYYLONGRDMYVDQEL--D--INRLSDYDVDHI | 880 |
| WP_029073316 | 789 | DSFVNQMLKLYK DFED EANKHLKg-EDa--KSKIRSERLKLYYTQMGKCMYTGKSL--D--IDRLDTYQVDHI | 860 |
| WP_031589969 | 789 | DSFVNQMLKLYK DFED EANKHLKg-EDa--KSKIRSERLKLYYTQMGKCMYTGKSL--D--IDRLDTYQVDHI | 860 |
| KDA45870 | 772 | EDRVQQIVKNLK ELPK ------P---S---NAELSDERKYLYCLONGRDMYTGAPL--D--YDHLQFYDVDHI | 833 |
| WP_039099354 | 789 | KRQVEQVYQNIS EL-- EIRNELK---D1-SNSALSNTRLFLYFMQGGRDMYTGDSL--N--IDRLSTYDIDHI | 856 |
| AKP02966 | 786 | RLQSKLLNKANG -LVP EELKKHKn--D------LSSERIMLYFLONGKSLYSEESL--N--INKLSDYQVDHI | 858 |
| WP_010991369 | 781 | RPRYKSLEKAIK EFGS QILKEHP--TD---NQELRNNRLYLYYLONGKDMYTGQDL--D--IHNLSNYDIDHI | 844 |
| WP_033838504 | 781 | RPRYKSLEKAIK EFGS QILKEHP--TD---NQELRNNRLYLYYLQNGKDMYTGQDL--D--IHNLSNYDIDHI | 844 |
| EHN60060 | 784 | RPRYKSLEKAIK EFGS QILKEHP--TD---NQELRNNRLYLYYLONGKDMYTGQDL--D--IHNLSNYDIDHI | 847 |
| EFR89594 | 550 | RPRYKSLEKAIK EFGS QILKEHP--TD---NQELKNNRLYLYYLONGKDMYTGQDL--D--IHNLSNYDIDHI | 613 |
| WP_038409211 | 781 | KPRFISLEKAIK EFGS QILKEHP--TD---NQCLKNDRLYLYYLONGKDMYTGKEL--D--IHNLSNYDIDHI | 844 |
| EFR95520 | 400 | KPRFISLEKAIK EFGS QILKEHP--TD---NQCLKNDRLYLYYLONGKDMYTGKEL--D--IHNLSNYDIDHI | 463 |
| WP_003723650 | 781 | KPRYKSLEKAIK EFGS QILKEHP--TD---NQELKNNRLYLYYLONGKDMYTGQEL--D--IHNLSNYDIDHI | 844 |
| WP_003727705 | 781 | KPRYKSLEKAIK DFGS QILKEHP--TD---NQELKNNRLYLYYLONGKDIYTGQEL--D--IHNLSNYDIDHI | 844 |
| WP_003730785 | 781 | KPRYKSLEKAIK DFGS QILKEHP--TD---NQELKNNRLYLYYLONGKDIYTGQEL--D--IHNLSNYDIDHI | 844 |
| WP_003733029 | 781 | KPRYKSLEKAIK EFGS QILKEHP--TD---NQELKNNRLYLYYLQNGKDMYTGQEL--D--IHNLSNYDIDHI | 844 |
| WP_003739838 | 781 | RPRYKSLEKAIK EFGS QILKEHP--TD---NQELRNNRLYLYYLONGKDMYTGQEL--D--IHNLSNYDIDHI | 844 |
| WP_014601172 | 781 | KPRYKSLEKAIK EFGS KILKEHP--TD---NQELKNNRLYLYYLONGKDMYTGQEL--D--IHNLSNYDIDHI | 844 |
| WP_023548323 | 781 | KPRYKSLEKAIK EFGS QILKEHP--TD---NQELKNNRLYLYYLONGKDMYTGQEL--D--IHNLSNYDIDHI | 844 |
| WP_031665337 | 781 | KPRYKSLEKAIK EFGS QILKEHP--TD---NQELKNNRLYLYYLONGKDMYTGQEL--D--IHNLSNYDIDHI | 844 |
| WP_031669209 | 781 | KPRYKSLEKAIK EFGS QILKEHP--TD---NQELKNNRLYLYYLONGKDMYTGQEL--D--IHNLSNYDIDHI | 844 |
| WP_033920898 | 781 | KPRYKSLEKAIK EFGS QILKEHP--TD---NQELKNNRLYLYYLONGKDMYTGQEL--D--IHNLSNYDIDHI | 844 |
| AKI42028 | 784 | KPRYKSLEKAIK EFGS KILKEHP--TD---NQELKNNRLYLYYLONGKDMYTGQEL--D--IHNLSNYDIDHI | 847 |
| AKI50529 | 784 | KPRYKSLEKAIK EFGS QILKEHP--TD---NQELKNNRLYLYYLONGKDMYTGQEL--D--IHNLSNYDIDHI | 847 |
| EFR83390 | 229 | RPRYKSLEKAIK EFGS QILKEHP--TD---NQELKNNRLYLYYLONGKDIYTGQEL--D--IHNLSNYDIDHI | 292 |
| WP_046323366 | 781 | KPRFTSLEKAIK ELGS QILKEHP--TD---NQGLKNDRLYLYYLONGKDMYTGQEL--D--IHNLSNYDIDHV | 844 |
| AKE81011 | 794 | RERMKRIEEGIK ELGS QILKEHP--VE---NTQLQNEKLYLYYLONGRDMYVDQEL--D--INRLSDYDVDHI | 857 |
| CUO82355 | 781 | ESKIKKLENVYK DEQT SVLEELKg-FDn--TKKISSDSLFLYFTQLGKCMYSGKKL--D--IDSLDKYQIDHI | 853 |
| WP_033162887 | 784 | ESKIAKLQKIYE NLQT QVYESLKK-EDa--KKRMETDALYLYYLQMGKSMYSGKPL--D--IDKLSTYQIDHI | 855 |
| AGZ01981 | 811 | RERMKRIEEGIK ELGS QILKEHP--VE---NTQLQNEKLYLYYLONGRDMYVDQEL--D--INRLSDYDVDHI | 874 |
| AKA60242 | 778 | RERMKRIEEGIK ELGS QILKEHP--VE---NTQLQNEKLYLYYLONGRDMYVDQEL--D--INRLSDYDVDAI | 841 |
| AKS40380 | 778 | RERMKRIEEGIK ELGS QILKEHP--VE---NTQLQNEKLYLYYLONGRDMYVDQEL--D--INRLSDYDVDHI | 841 |
| 4UN5_B | 782 | RERMKRIEEGIK ELGS QILKEHP--VE---NTQLQNEKLYLYYLONGRDMYVDQEL--D--INRLSDYDVDAI | 845 |
| WP_010922251 | 842 | 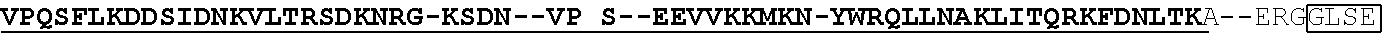 | 910 |
| WP_039695303 | 852 | IPQAFIKDDSIDNRVLTSSAKNRG-KSDD--VP S--LDIVRARKA-EWVRLYKSGLISKRKFDNLTKA--ERGGLTE | 920 |
| WP_045635197 | 844 | IPQAFIKDDSLDNRVLTSSKDNRG-KSDN--VP S--IEVVQKRKA-FWQQLLDSKLISERKENNLTKA--ERGGLDE | 912 |
| 5AXW_A | 562 | IPRSVSFDNSFNNKVLVKQEEASK-KGNR--TP Fqy-LSSSDSKI-SYETFKKHILNLAKGKGRISKTk-KEYLLEE | 632 |
| WP_009880683 | 526 | VPQSFLKDDSIDNKVLTRSDKNRG-KSDN--VP S--EEVVKKMKN-YWKQLLNAKLITORKFDNLTKA--ERGGLSE | 594 |
| WP_010922251 | 842 | VPQSFLKDDSIDNKVLTRSDKNRG-KSDN--VP S--EEVVKKMKN-YWROLLNAKLITQRKFDNLTKA--ERGGLSE | 910 |
| WP_011054416 | 842 | VPQSFLKDDSIDNKVLTRSDKNRG-KSDN--VP S--EEVVKKMKN-YWROLLNAKLITORKFDNLTKA--ERGGLSE | 910 |
| WP_011284745 | 842 | VPQSFIKDDSIDNKVLTRSDKNRG-KSNN--VP S--EEVVKKMKN-YWROLLNAKLITORKFDNLTKA--ERGGLSE | 910 |
| WP_011285506 | 842 | VPQSFLKDDSIDNKVLTRSDKNRG-KSDN--VP S--EEVVKKMKN-YWROLLNAKLITORKFDNLTKA--ERGGLSE | 910 |
| WP_011527619 | 842 | VPQSFIKDDSIDNKVLTRSDKNRG-KSDN--VP S--EEVVKKMKN-YWRQLLNAKLITQRKFDNLTKA--ERGGLSE | 910 |
| WP_012560673 | 842 | VPQSFLKDDSIDNKVLTRSDKNRG-KSDN--VP S--EEVVKKMKN-YWKOLLNAKLITQRKFDNLTKA--ERGGLSE | 910 |
| WP_014407541 | 841 | VPQSFIKDDSIDNKVLTRSDKNRG-KSDN--VP S--EEVVKKMKN-YWROLLNAKLITQRKFDNLTKA--ERGGLSE | 909 |
| WP_020905136 | 842 | VPQSFIKDDSIDNKVLTRSDKNRG-KSDN--VP S--EEVVKKMKN-YWROLLNAKLITORKFDNLTKA--ERGGLSE | 910 |
| WP_023080005 | 841 | VPQSFLKDDSIDNKVLTRSDKNRG-KSDN--VP S--EEVVKKMKN-YWROLLNAKLITORKFDNLTKA--ERGGLSE | 909 |
| WP_023610282 | 841 | VPQSFLKDDSIDNKVLTRSDKNRG-KSDN--VP S--EEVVKKMKN-YWROLLNAKLITQRKFDNLTKA--ERGGLSE | 909 |
| WP_030125963 | 842 | VPQSFIKDDSIDNKVLTRSDKNRG-KSDN--VP S--EEVVKKMKN-YWROLLNAKLITORKFDNLTKA--ERGGLSE | 910 |
| WP_030126706 | 842 | VPQSFLKDDSIDNKVLTRSDKNRG-KSDN--VP S--EEVVKKMKN-YWROLLNAKLITORKFDNLTKA--ERGGLSE | 910 |
| WP_031488318 | 842 | VPQSFIKDDSIDNKVLTRSDKNRG-KSDN--VP S--EEVVKKMKN-YWKOLLNAKLITQRKFDNLTKA--ERGGLSE | 910 |
| WP_032460140 | 842 | VPQSFLKDDSIDNKVLTRSDKNRG-KSDN--VP S--EEVVKKMKN-YWKOLLNAKLITQRKFDNLTKA--ERGGLSE | 910 |
| WP_032461047 | 842 | VPQSFLKDDSIDNKVLTRSDKNRG-KSDN--VP S--EEVVKKMKN-YWKQLLNAKLITORKFDNLTKA--ERGGLSE | 910 |
| WP_032462016 | 842 | VPQSFLKDDSIDNKVLTRSDKNRG-KSDN--VP S--EEVVKKMKN-YWROLLNAKLITORKFDNLTKA--ERGGLSE | 910 |
| WP_032462936 | 842 | VPQSFLKDDSIDNKVLTRSDKNRG-KSDN--VP S--EEVVKKMKN-YWKOLLNAKLITQRKFDNLTKA--ERGGLSE | 910 |
| WP_032464890 | 842 | VPQSFIKDDSIDNKVLTRSDKNRG-KSDN--VP S--EEVVKKMKN-YWROLLNAKLITQRKFDNLTKA--ERGGLSE | 910 |
| WP_033888930 | 667 | VPQSFLKDDSIDNKVLTRSDKNRG-KSDN--VP S--EEVVKKMKN-YWRQLLNAKLITORKFDNLTKA--ERGGLSE | 735 |
| WP_038431314 | 842 | VPQSFLKDDSIDNKVLIRSDKNRG-KSDN--VP S--EEVVKKMKN-YWROLLNAKLITQRKFDNLTKA--ERGGLSE | 910 |
| WP_038432938 | 841 | VPQSFLKDDSIDNKVLTRSDKNRG-KSDN--VP S--EEVVKKMKN-YWROLLNAKLITORKFDNLTKA--ERGGLSE | 909 |
| WP_038434062 | 842 | VPQSFLKDDSIDNKVLTRSDKNRG-KSDN--VP S--EEVVKKMKN-YWKOLLNAKLITORKFDNLTKA--ERGGLSE | 910 |
| BAQ51233 | 753 | VPQSFLKDDSIDNKVLTRSDKNRG-KSDN--VP S--EEVVKKMKN-YWROLLNAKLITORKFDNLTKA--ERGGLSE | 821 |
| KGE60162 | 17 | VPQSFLKDDSIDNKVLTRSDKNRG-KSDN--VP S--EEVVKKMKN-YWROLLNAKLITORKFDNLTKA--ERGGLSE | 85 |
| KGE60856 | | --------------------------------- -------------------------------------------- | |
| WP_002989955 | 842 | VPQSFIKDDSIDNKVLTRSDKNRG-KSDN--VP S--EEVVKKMKN-YWROLLNAKLITQRKFDNLTKA--ERGGLSE | 910 |
| WP_003030002 | 841 | IPQAFIKDNSLDNRVLTRSDKNRG-KSDD--VP S--IEVVHEMKS-FWSKLLSVKLITQRKFDNLTKA--ERGGLTE | 909 |
| WP_003065552 | 852 | IPQAFIKDDSIDNRVLTSSAKNRG-KSDD--VP S--LDIVRARKA-EWVRLYKSGLISKRKFDNLTKA--ERGGLTE | 920 |
| WP_001040076 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040078 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040080 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040081 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040083 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040085 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040087 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040088 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040089 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040090 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040091 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040092 | 847 | VPQAFIKDDSIDNRVLVSSAKNRG-KSDD--VP S--IDIVKARKA-FWKKLLDAKLISQRKYDNLTKA--ERGGLTP | 915 |
| WP_001040094 | 847 | IPQAFIKDDSIDNRVLVSSAKNRG-KSDD--VP S--VEIVKDCKV-FWKKLLDAKLMSQRKYDNLTKA--ERGGLTS | 915 |
| WP_001040095 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040096 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040097 | 847 | IPQAFIKDDSIDNRVLVSSAKNRG-KSDD--VP S--VEIVKDCKV-FWKKLLDAKLMSQRKYDNLTKA--ERGGLTS | 915 |
| WP_001040098 | 847 | IPQAFIKDDSIDNRVLVSSAKNRG-KSDD--VP S--VEIVKDCKV-FWKKLLDAKLMSQRKYDNLTKA--ERGGLTS | 915 |
| WP_001040099 | 847 | IPQAFIKDDSIDNRVLVSSAKNRG-KSDD--VP S--VEIVKDCKV-FWKKLLDAKLMSORKYDNLTKA--ERGGLTS | 915 |
| WP_001040100 | 847 | IPQAFIKDDSIDNRVLVSSAKNRG-KSDD--VP S--VEIVKDCKV-FWKKLLDAKLMSQRKYDNLTKA--ERGGLTS | 915 |
| WP_001040104 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040105 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_001040106 | 847 | VPQAFIKDDSIDNRVLVSSAKNRG-KSDN--VP S--IDIVKARKA-FWKKLLDAKLMSQRKYDNLTKA--ERGGLTS | 915 |
| WP_001040107 | 847 | VPQAFIKDDSIDNRVLVSSAKNRG-KSDN--VP S--IDIVKARKA-FWKKLLDAKLMSORKYDNLTKA--ERGGLTS | 915 |
| WP_001040108 | 847 | VPQAFIKDDSIDNRVLVSSAKNRG-KSDN--VP S--IDIVKARKA-FWKKLLDAKLMSQRKYDNLTKA--ERGGLTS | 915 |
| WP_001040109 | 847 | VPQAFIKDDSIDNRVLVSSAKNRG-KSDN--VP S--IDIVKARKA-FWKKLLDAKLMSORKYDNLTKA--ERGGLTS | 915 |
| WP_001040110 | 847 | VPQAFIKDDSIDNRVLVSSAKNRG-KSDN--VP S--IDIVKARKA-FWKKLLDAKLMSQRKYDNLTKA--ERGGLTS | 915 |
| WP_015058523 | 847 | VPQAFIKDDSIDNRVLVSSAKNRG-KSDD--VP S--IDIVKARKA-FWKKLLDAKLISQRKYDNLTKA--ERGGLTP | 915 |
| WP_017643650 | 847 | IPQAFIKDDSIDNRVLVSSAKNRG-KSDD--VP S--VEIVKDCKV-FWKKLLDAKLMSQRKYDNLTKA--ERGGLTS | 915 |
| WP_017647151 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_017648376 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_017649527 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_017771611 | 847 | VPQAFIKDDSIDNRVLVSSAKNRG-KSDN--VP S--IDIVKARKA-FWKKLLDAKLMSQRKYDNLTKA--ERGGLTS | 915 |
| WP_017771984 | 847 | IPQAFIKDDSIDNRVLVSSAKNRG-KSDD--VP S--LEIVKDCKV-FWKKLLDAKLMSORKYDNLTKA--ERGGLTS | 915 |
| CFQ25032 | 847 | IPQAFIKDDSIDNRVLVSSAKNRG-KSDD--VP S--LEIVKDCKV-FWKKLLDAKLMSQRKYDNLTKA--ERGGLTS | 915 |
| CFV16040 | 847 | IPQAFIKDDSIDNRVLVSSAKNRG-KSDD--VP S--LEIVKDCKV-FWKKLLDAKLMSQRKYDNLTKA--ERGGLTS | 915 |
| KLJ37842 | 847 | IPQAFIKDDSIDNRVLVSSAKNRG-KSDD--VP S--LEIVKDCKV-FWKKLLDAKLMSQRKYDNLTKA--ERGGLTS | 915 |
| KLJ72361 | 847 | IPQAFIKDDSIDNRVLVSSAKNRG-KSDD--VP S--LEIVKDCKV-FWKKLLDAKLMSQRKYDNLTKA--ERGGLTS | 915 |
| KLL20707 | 861 | IPQAFIKDDSIDNRVLVSSAKNRG-KSDD--VP S--LEIVKDCKV-FWKKLLDAKLMSQRKYDNLTKA--ERGGLTS | 929 |
| KLL42645 | 847 | VPQAFIKDDSIDNRVLVSSAKNRG-KSDN--VP S--IDIVKARKA-FWKKLLDAKLMSQRKYDNLTKA--ERGGLTS | 915 |
| WP_047207273 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_047209694 | 847 | IPQAFIKDDSIDNRVLVSSAKNRG-KSDD--VP S--VEIVKDCKV-FWKKLLDAKLMSQRKYDNLTKA--ERGGLTS | 915 |
| WP_050198062 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_050201642 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_050204027 | 847 | VPQAFIKDDSIDNRVLVSSAKNRG-KSDN--VP S--IDIVKARKA-FWKKLLDAKLMSQRKYDNLTKA--ERGGLTS | 915 |
| WP_050881965 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_050886065 | 847 | IPQAFIKDDSIDNRVLVSSAKNRG-KSDD--VP S--LEIVKDCKV-FWKKLLDAKLMSQRKYDNLTKA--ERGGLTS | 915 |
| AHN30376 | 847 | VPQAFIKDDSIDNRVLVSSAKNRG-KSDD--VP S--IDIVKARKA-FWKKLLDAKLISQRKYDNLTKA--ERGGLTP | 915 |
| EAO78426 | 847 | IPQAFIKDDSIDNRVLVSSAKNRG-KSDD--VP S--LEIVKDCKV-FWKKLLDAKLMSQRKYDNLTKA--ERGGLTS | 915 |
| CCW42055 | 847 | IPQAFIKDDSIDNRVLVSSAKNRG-KSDD--VP S--LEIVKDCKV-FWKKLLDAKLMSQRKYDNLTKA--ERGGLTS | 915 |
| WP_003041502 | 841 | IPQAYIKDDSFDNRVLTSSSENRG-KSDN--VP S--IEVVCARKA-DWMRLRKAGLISQRKFDNLTKA--ERGGLTE | 909 |
| WP_037593752 | 842 | IPQAFIKDNSLDNRVLTRSDKNRG-KSDD--VP S--IEVVHEMKS-FWSKLLSVKLITQRKFDNLTKA--ERGGLTE | 910 |
| WP_049516684 | 842 | IPQAFIKDNSLDNRVLTRSDKNRG-KSDD--VP S--IEVVHEMKS-FWSKLLSVKLITQRKFDNLTKA--ERGGLTE | 910 |
| GAD46167 | 841 | IPQAFIKDNSLDNRVLTRSDKNRG-KSDD--VP S--IEVVHEMKS-FWSKLLSVKLITQRKFDNLTKA--ERGGLTE | 909 |
| WP_018363470 | 850 | IPQAFIKDDSIDNRVLTSSAKNRG-KSDD--VP S--LGIVRARKA-EWVRLYKSGLISKRKFDNLTKA--ERGGLTE | 918 |
| WP_003043819 | 851 | VPQSFIKDDSIDNKVLTRSVENRG-KSDN--VP S--EEVVKKMKN-YWROLLNAKLITORKFDNLTKA--ERGGLSE | 919 |
| WP_006269658 | 841 | IPQAFIKDNSLDNRVLTRSDKNRG-KSDD--VP S--IEVVHEMKS-FWSKLLSVKLITORKFDNLTKA--ERGGLTE | 909 |
| WP_048800889 | 841 | IPQAFIKDDSIDNRVLTSSAKNRG-KSDN--VP N--LEVVCDRKA-DWIRLREAGLISQRKFDNLTKA--ERGGLTE | 909 |
| WP_012767106 | 841 | VPQSFIKDDSIDNKILTRSDKNRG-KSDN--VP S--EEVVKKMKN-YWROLLNAKLITORKFDNLTKA--ERGGLSE | 909 |
| WP_014612333 | 841 | VPQSFIKDDSIDNKVLTRSDKNRG-KSDN--VP S--EEVVKKMKN-YWROLLNAKLITQRKFDNLTKA--ERGGLSE | 909 |
| WP_015017095 | 841 | VPQSFIKDDSIDNKVLTRSDKNRG-KSDD--VP S--EEVVKKMKN-YWROLLNAKLITQRKFDNLTKA--ERGGLSE | 909 |
| WP_015057649 | 841 | VPQSFIKDDSIDNKVLTRSDKNRG-KSDN--VP S--EEVVKKMKN-YWRQLLNAKLITORKFDNLTKA--ERGGLSE | 909 |
| WP_048327215 | 841 | VPQSFIKDDSIDNKVLTRSDKNRG-KSDD--VP S--EEVVKKMKN-YWRQLLNAKLITORKFDNLTKA--ERGGLSE | 909 |
| WP_049519324 | 841 | VPQSFIKDDSIDNKVLTRSDKNRG-KSDN--VP S--EEVVKKMKN-YWROLLNAKLITORKFDNLTKA--ERGGLSE | 909 |
| WP_012515931 | 841 | IPQSFIKNNSIDNKVLTSQGANRG-KLDN--VP S--EAIVRKMKG-YWQSLLRAGAISKQKFDNLTKA--ERGGLTQ | 909 |
| WP_021320964 | 841 | IPQSFIKNNSIDNKVLTSQGANRG-KLDN--VP S--EAIVRKMKG-YWQSLLRAGAISKQKFDNLTKA--ERGGLTQ | 909 |
| WP_037581760 | 841 | IPQSFIKNNSIDNKVLTSQGANRG-KLDN--VP S--EAIVRKMKG-YWQSLLRAGAISKQKFDNLTKA--ERGGLTQ | 909 |
| WP_004232481 | 849 | IPQAFIKDNSIDNRVLTSSAKNRG-KSDD--VP S--IEIVRNRKS-YWYKLYKSGLISKRKFDNLTKA--ERGGLTE | 917 |
| WP_009854540 | 850 | IPQAFIKDDSIDNRVLTSSAKNRG-KSDD--VP S--LDIVRARKA-EWVRLYKSGLISKRKFDNLTKA--ERGGLTE | 918 |
| WP_012962174 | 850 | IPQAFIKDDSIDNRVLTSSAKNRG-KSDD--VP S--LDIVHDRKA-DWIRLYKSGLISKRKFDNLTKA--ERGGLTE | 918 |
| WP_039695303 | 852 | IPQAFIKDDSIDNRVLTSSAKNRG-KSDD--VP S--LDIVRARKA-EWVRLYKSGLISKRKFDNLTKA--ERGGLTE | 920 |
| WP_014334983 | 849 | IPQAFIKDNSIDNKVLTSSAKNRG-KSDD--VP S--IEIVRNRRS-YWYKLYKSGLISKRKFDNLTKA--ERGGLTE | 917 |
| WP_003099269 | 842 | IPQSFIKDNSIDNTVLTTQASNRG-KSDN--VP N--IETVNKMKS-FWYKQLKSGAISQRKFDHLTKA--ERGALSD | 910 |
| AHY15608 | 842 | IPQSFIKDNSIDNTVLTTQASNRG-KSDN--VP N--IETVNKMKS-FWYKQLKSGAISQRKFDHLTKA--ERGALSD | 910 |
| AHY17476 | 842 | IPQSFIKDNSIDNTVLTTQASNRG-KSDN--VP N--IETVNKMKS-FWYKQLKSGAISQRKFDHLTKA--ERGALSD | 910 |
| ESR09100 | | --------------------------------- -------------------------------------------- | |
| AGM98575 | 842 | IPQSFIKDNSIDNTVLTTQASNRG-KSDN--VP N--IETVNKMKS-FWYKQLKSGAISQRKFDHLTKA--ERGALSD | 910 |
| ALF27331 | 842 | IPQAFIKDNSIDNRVLTSSKENRG-KSDD--VP S--KDVVRKMKS-YWSKLLSAKLITORKFDNLTKA--ERGGLTD | 910 |
| WP_018372492 | 858 | VPRSYIKNDSFDNKVLTTSKGNRK-KLDD--VP A--KEVVEKMEN-TWRRLHAAGLISDIKLSYLMKGe-----LTE | 923 |
| WP_045618028 | 845 | IPQAFIKDDSLDNRVLISSKDNRG-KSDN--VP S--LEIVOKRKA-FWQQLLDSKLISERKENNLTKA--ERGGLDE | 913 |
| WP_045635197 | 844 | IPQAFIKDDSLDNRVLISSKDNRG-KSDN--VP S--IEVVQKRKA-FWQQLLDSKLISERKENNLTKA--ERGGLDE | 912 |
| WP_002263549 | 842 | IPQAFIKDNSIDNRVLTSSKENRG-KSDD--VP S--KDVVRKMKS-YWSKLLSAKLITQRKFDNLTKA--ERGGLTD | 910 |
| WP_002263887 | 842 | IPQAFIKDNSIDNRVLTSSKENRG-KSDD--VP S--KDVVRKMKS-YWSKLLSAKLITORKFDNLTKA--ERGGLTD | 910 |
| WP_002264920 | 842 | IPQAFIKDNSIDNRVLTSSKENRG-KSDD--VP S--KDVVRKMKS-YWSKLLSAKLITQRKFDNLTKG--ERGGLTD | 910 |
| WP_002269043 | 842 | IPQAFIKDNSIDNRVLTSSKENRG-KSDD--VP S--KDVVRKMKS-YWSKLLSAKLITQRKFDNLTKA--ERGGLTD | 910 |
| WP_002269448 | 842 | IPQAFIKDNSIDNRVLTSSKENRG-KSDD--VP S--EDVVRKMKS-YWSKLLSAKLITORKFDNLTKA--ERGGLTD | 910 |
| WP_002271977 | 842 | IPQAFIKDNSIDNRVLTSSKENRG-KSDD--VP S--KDVVRKMKS-YWSKLLSAKLITORKFDNLTKA--ERGGLTD | 910 |
| WP_002272766 | 842 | IPQAFIKDNSIDNRVLTSSKENRG-KSDD--VP S--KDVVRKMKP-YWSKLLSAKLITORKFDNLTKA--ERGGLTD | 910 |
| WP_002273241 | 842 | IPQAFIKDNSIDNRVLTSSKENRG-KSDD--VP S--KDVVRKMKS-YWSKLLSAKLITQRKFDNLTKA--ERGGLTD | 910 |
| WP_002275430 | 842 | IPQAFIKDNSIDNRVLTSSKENRG-KSDD--VP S--KDVVRKMKP-YWSKLLSAKLITORKFDNLTKA--ERGGLTD | 910 |
| WP_002276448 | 842 | IPQAFIKDNSIDNRVLTSSKENRG-KSDD--VP S--KDVVRKMKS-YWSKLLSAKLITORKFDNLTKA--ERGGLTD | 910 |
| WP_002277050 | 847 | IPQAFIKDNSIDNRVLTSSKANRG-KSDD--VP S--EDVVNRMRP-FWNKLLSSGLISQRKYNNLTKK--E---LTP | 912 |
| WP_002277364 | 842 | IPQAFIKDNSIDNRVLTSSKENRG-KSDD--VP S--KNVVRKMKS-YWSKLLSAKLITQRKFDNLTKA--ERGGLTD | 910 |
| WP_002279025 | 842 | IPQAFIKDNSIDNRVLTSSKENRG-KSDD--VP S--KDVVRKMKP-YWSKLLSAKLITORKFDNLTKA--ERGGLTD | 910 |
| WP_002279859 | 842 | IPQAFIKDNSIDNRVLTSSKENRG-KSDD--VP S--KDVVRKMKS-YWSKLLSAKLITQRKFDNLTKG--ERGGLTD | 910 |
| WP_002280230 | 842 | IPQAFIKDNSIDNRVLTSSKENRG-KSDD--VP S--KDVVRKMKS-YWSKLLSAKLITQRKFDNLTKA--ERGGLTD | 910 |
| WP_002281696 | 842 | IPQAFIKDNSIDNRVLTSSKENRG-KSDD--VP S--KNVVRKMKS-YWSKLLSAKLITQRKFDNLTKA--ERGGLTD | 910 |
| WP_002282247 | 847 | IPQAFIKDNSIDNRVLTSSKANRG-KSDD--VP S--EDVVNRMRP-FWNKLLSSGLISQRKYNNLTKK--E---LTL | 912 |
| WP_002282906 | 842 | IPQAFIKDNSIDNRVLTSSKENRG-KSDD--VP S--KDVVRKMKS-YWSKLLSAKLITORKFDNLTKA--ERGGLTD | 910 |
| WP_002283846 | 842 | IPQAFIKDNSIDNRVLTSSKENRG-KSDD--VP S--KNVVRKMKS-YWSKLLSAKLITQRKFDNLTKA--ERGGLTD | 910 |
| WP_002287255 | 842 | IPQAFIKDNSIDNRVLTSSKENRG-KSDD--VP S--KNVVRKMKS-YWSKLLSAKLITQRKFDNLTKA--ERGGLTD | 910 |
| WP_002288990 | 842 | IPQAFIKDNSIDNRVLTSSKENRG-KSDD--VP S--KDVVRKMKS-YWSKLLSAKLITQRKFDNLTKA--ERGGLTD | 910 |
| WP_002289641 | 842 | IPQAFIKDNSIDNRVLTSSKENRG-KSDD--VP S--KDVVRKMKS-YWSKLLSAKLITORKFDNLTKA--ERGGLTD | 910 |
| WP_002290427 | 842 | IPQAFIKDNSIDNRVLTSSKENRG-KSDD--VP S--KDVVRKMKS-YWSKLLSAKLITORKFDNLTKG--ERGGLTD | 910 |
| WP_002295753 | 842 | IPQAFIKDNSIDNRVLTSSKENRG-KSDD--VP S--KDVVRKMKS-YWSKLLSAKLITQRKFDNLTKA--ERGGLTD | 910 |
| WP_002296423 | 842 | IPQAFIKDNSIDNRVLTSSKENRG-KSDD--VP S--KNVVRKMKS-YWSKLLSAKLITORKFDNLTKA--ERGGLTD | 910 |
| WP_002304487 | 856 | IPQAFIKDNSIDNRVLTRSDKNRG-KSDD--VP S--EEVVHKMKP-FWSKLLSAKLITORKFDNLTKA--ERGGLTD | 924 |
| WP_002305844 | 842 | IPQAFIKDNSIDNRVLTSSKENRG-KSDD--VP S--KDVVRKMKS-YWSKLLSAKLITQRKFDNLTKA--ERGGLTD | 910 |
| WP_002307203 | 842 | IPQAFIKDNSIDNRVLTSSKENRG-KSDD--VP S--KDVVRKMKS-YWSKLLSAKLITQRKFDNLTKA--ERGGLTD | 910 |
| WP_002310390 | 842 | IPQAFIKDNSIDNRVLTSSKENRG-KSDD--VP S--KDVVRKMKS-YWSKLLSAKLITORKFDNLTKG--ERGGLTD | 910 |
| WP_002352408 | 842 | IPQAFIKDNSIDNRVLTSSKENRG-KSDD--VP S--KDVVRKMKS-YWSKLLSAKLITQRKFDNLTKA--ERGGLTD | 910 |
| WP_012997688 | 842 | IPQAFIKDNSIDNRVLTSSKENRG-KSDD--VP S--KNVVRKMKS-YWSKLLSAKLITQRKFDNLTKA--ERGGLTD | 910 |
| WP_014677909 | 842 | IPQAFIKDNSIDNRVLTSSKENRG-KSDD--VP S--KDVVRKMKS-YWSKLLSAKLITORKFDNLTKA--ERGGLTD | 910 |
| WP_019312892 | 842 | IPQAFIKDNSIDNRVLTSSKENRG-KSDD--VP S--KNVVRKMKS-YWSKLLSAKLITQRKFDNLTKA--ERGGLTD | 910 |
| WP_019313659 | 842 | IPQAFIKDNSIDNRVLTSSKENRG-KSDD--VP S--KDVVRKMKS-YWSKLLSAKLITORKFDNLTKA--ERGGLTD | 910 |
| WP_019314093 | 842 | IPQAFIKDNSIDNRVLTSSKENRG-KSDD--VP S--KDVVRKMKS-YWSKLLSAKLITORKFDNLTKA--ERGGLTD | 910 |
| WP_019315370 | 842 | IPQAFIKDNSIDNRVLTSSKENRG-KSDD--VP S--KDVVRKMKP-YWSKLLSAKLITQRKFDNLTKA--ERGGLTD | 910 |
| WP_019803776 | 842 | IPQAFIKDNSIDNRVLTSSKENRG-KSDD--VP S--KDVVRKMKS-YWSKLLSAKLITORKFDNLTKA--ERGGLTD | 910 |
| WP_019805234 | 842 | IPQAFIKDNSIDNRVLTSSKENRG-KSDD--VP S--KDVVRKMKS-YWSKLLSAKLITQRKFDNLTKG--RGGLTD | 910 |
| WP_024783594 | 842 | IPQAFIKDNSIDNRVLTSSKENRG-KSDD--VP S--KDVVRKMKS-YWSKLLSAKLITQRKFDNLTKA--ERGGLTD | 910 |
| WP_024784288 | 847 | IPQAFIKDNSIDNRVLTSSKANRG-KSDD--VP S--EDVVNRMRP-FWNKLLSSGLISQRKYNNLIKK--E---LTL | 912 |
| WP_024784666 | 842 | IPQAFIKDNSIDNRVLTSSKENRG-KSDD--VP S--KNVVRKMKS-YWSKLLSAKLITQRKFDNLTKA--ERGGLTD | 910 |
| WP_024784894 | 842 | IPQAFIKDNSIDNRVLTSSKENRG-KSDD--VP S--KDVVRKMKS-YWSKLLSAKLITQRKFDNLTKA--ERGGLTD | 910 |
| WP_024786433 | 847 | IPQAFIKDNSIDNRVLTSSKANRG-KSDD--VP S--EDVVNRMRP-FWNKLLSSGLISQRKYNNLTKK--E---LTL | 912 |
| WP_049473442 | 842 | IPQAFIKDNSIDNRVLTSSKENRG-KSDD--VP S--KDVVRKMKP-YWSKLLSAKLITORKFDNLTKA--ERGGLTD | 910 |
| WP_049474547 | 842 | IPQAFIKDNSIDNRVLTSSKENRG-KSDD--VP S--KDVVRKMKS-YWSKLLSAKLITORKFDNLTKA--ERGGLTD | 910 |
| EMC03581 | 835 | IPQAFIKDNSIDNRVLTSSKENRG-KSDD--VP S--KNVVRKMKS-YWSKLLSAKLITORKFDNLTKA--ERGGLTD | 903 |
| WP_000428612 | 847 | VPQAFIKDDSLDNRVLTSLKDNRG-KSDN--VP S--LEVVEKMKT-FWQQLLDSKLISYRKENNLTKA--ERGGLDE | 915 |
| WP_000428613 | 845 | VPQAFIKDDSLDNRVLTSLKDNRG-KSDN--VP S--IEVVQKRKA-FWQQLLDSKLISERKENNLTKA--ERGGLDE | 913 |
| WP_049523028 | 840 | IPQAFIKDDSLDNRVLTSSKDNRG-KSDN--VP S--LEIVEKMKG-FWQQLLDSKLISERKENNLTKA--ERGGLDE | 908 |
| WP_003107102 | 811 | IPQSFIKDNSIDNIVLTSQESNRG-KSDN--VP Y--IAIVNKMKS-YWQHQLKSGAISQRKFDNLTKA--ERGGLSE | 879 |
| WP_054279288 | 843 | IPRSFIKDDSIDNKVLTRSEHNRG-KTDN--VP S--IEVVKRMKP-YWQKLLDTKVISQRKFDNLTKA--ERGGLQE | 911 |
| WP_049531101 | 845 | IPQAFIKDDSLDNRVLTSSKDNRG-KSDN--VP S--LEVVQKRKA-FWQQLLESKLISERKENNLTKA--ERGGLNE | 913 |
| WP_049538452 | 845 | IPQAFIKDDSLDNRVLTSSKENRG-KSDN--VP C--LEVVDKMKV-FWQQLLDFKLISYRKENNLTKA--ERGGLDE | 913 |
| WP_049549711 | 845 | IPQAFIKDDSLDNRVLTSSKDNRG-KSDN--VP S--LEVVOKRKA-FWQQLLDSKLISERKENNLTKAerERDGLNE | 915 |
| WP_007896501 | 847 | IPQSFIKDNSIDNLVLTTQKANRG-KSDN--VP S--IEVVRDMKDrVWRRQLANGAISROKFDHLTKA--ERGGLAD | 916 |
| EFR44625 | 799 | IPQSFIKDNSIDNLVLTTQKANRG-KSDN--VP S--IEVVRDMKDrVWRRQLANGAISRQKFDHLTKA--ERGGLAD | 868 |
| WP_002897477 | 844 | IPQAFIKDDSIDNRVLTSSKDNRG-KSDN--VP S--LEVVOKRKA-FWQQLLDSKLISERKENNLTKA--ERGGLDE | 912 |
| WP_002906454 | 844 | IPQAFIKDDSLDNRVLTSSKDNRG-KSDN--VP S--IEVVQKRKA-FWQQLLDSKLISERKENNLTKA--KRGGLDE | 912 |
| WP_009729476 | 845 | IPQAFIKDDSLDNRVLTSSKDNRG-KSDN--VP S--LEVVDKMKV-FWQQLLDSKLISYRKENNLTKA--ERGGLNE | 913 |
| CQR24647 | 844 | IPQSFIKDNSLDNRVLINSKSNRG-KSDN--VP S--NEVVKRMKG-FWLKOLDAKLISQRKFDNLTKA--ERGGLSA | 912 |
| WP_000066813 | 847 | IPQAFIKDDSLDNRVLTSSKDNRG-KSDN--VP S--LEVVEKMKA-FWQQLLDSKLISERKENNLTKAerERGGLNE | 917 |
| WP_009754323 | 845 | IPQAFIKDDSLDNRVLTSSKDNRG-KSDN--VP S--LEVVKKRKA-FWQQLLDSKLISERKENNLTKA--ERGGLDE | 913 |
| WP_044674937 | 844 | IPQAFIKDDSLDNKVLTKSAKNRG-KSDD--VP S--LEIVHKKKN-FWKOLLDSQLISQRKFDNLTKA--ERGGLTN | 912 |
| WP_044676715 | 846 | IPQAFIKDDSLDNKVLTKSAKNRG-KSDD--VP S--LEIVHKKKN-FWKOLLDSQLISQRKFDNLTKA--ERGGLIN | 914 |
| WP_044680361 | 846 | IPQAFIKDDSLDNKVLTKSAKNRG-KSDD--VP S--LEIVHKKKN-FWKQLLDSQLISQRKFDNLTKA--ERGGLIN | 914 |
| WP_044681799 | 844 | IPQAFIKDDSLDNKVLTKSAKNRG-KSDD--VP S--LEIVHKKKN-FWKQLLDSQLISQRKFDNLTKA--ERGGLIN | 912 |
| WP_049533112 | 841 | IPQAFIKDDSFDNRVLTSSSENRG-KSDN--VP S--IEVVRARKA-DWMRLRKAGLISQRKFDNLTKA--ERGGLTE | 909 |
| WP_029090905 | 824 | LPQSYIKDNSIENLALVKKVENQR-KKDS11LN S---SIINQNYS-RWEQLKNAGLIGEKKERNLTRTK-----ITD | 890 |
| WP_006506696 | 850 | VPQSLVKDDSFDNRVLVVPSENQR-KLDD1vVP ---FDIRDKMYR-FWKLLFDHELISPKKFYSLIKTe-----YTE | 916 |
| AIT42264 | 842 | VPQSFLKDDSIDNKVLTRSDKNRG-KSDN--VP S--EEVVKKMKN-YWROLLNAKLITQRKFDNLTKA--ERGGLSE | 910 |
| WP_034440723 | 849 | IPRSFITDNSFDNLVLTSSTVNRG-KLDN--VP Sp--DIVRQQKG-FWKOLLRAGLMSQRKENNLTKGK-----LTD | 914 |
| AKQ21048 | 842 | VPQSFLKDDSIDNKVLTRSDKNRG-KSDN--VP S--EEVVKKMKN-YWROLLNAKLITORKFDNLTKA--ERGGLSE | 910 |
| WP_004636532 | 846 | IPQSFTTDNSIDNKVLVSRTKNQGnKSDD--VP S--INIVHKMKP-FWRQLHKAGLISDRKFKNLTKA--EHGGLTE | 915 |
| WP_002364836 | 853 | IPQSFMKDDSLDNLVLVGSTENRG-KSDD--VP S--KEVVKDMKA-YWEKLYAAGLISQRKFORLTKG--EQGGLTL | 921 |
| WP_016631044 | 804 | IPQSFMKDDSLDNLVLVGSTENRG-KSDD--VP S--KEVVKDMKA-YWEKLYAAGLISQRKFORLTKG--EQGGLTL | 872 |
| EMS75795 | 589 | IPRSFIVDNSIDNKVLVSSKENRL-KMDD--VP D--QKVVIRMRR-YWEKLLRANLISERKFAYLTKLe-----LTP | 654 |
| WP_002373311 | 853 | IPQSFMKDDSLDNLVLVGSTENRG-KSDD--VP S--KKVVKKMKA-YWEKLYAAGLISQRKFORLTKG--EQGGLTL | 921 |
| WP_002378009 | 853 | IPQSFMKDDSLDNLVLVGSTENRG-KSDD--VP S--KEVVKDMKA-YWEKLYAAGLISQRKFORLTKG--EQGGLTL | 921 |
| WP_002407324 | 853 | IPQSFMKDDSLDNLVLVGSTENRG-KSDD--VP S--KEVVKDMKA-YWEKLYAAGLISQRKFORLTKG--EQGGLTL | 921 |
| WP_002413717 | 853 | IPQSFMKDDSLDNLVLVGSTENRG-KSDD--VP S--KEVVKDMKA-YWEKLYAAGLISQRKFORLTKG--EQGGLTL | 921 |
| WP_010775580 | 855 | IPQSFMKDDSLDNLVLVGSTENRG-KSDD--VP S--KEVVKKMKA-YWEKLYAAGLISQRKFORLTKG--EQGGLTL | 923 |
| WP_010818269 | 853 | IPQSFMKDDSLDNLVLVGSTENRG-KSDD--VP S--KEVVKDMKA-YWEKLYAAGLISQRKFQRLTKG--EQGGLTL | 921 |
| WP_010824395 | 853 | IPQSFMKDDSLDNLVLVGSTENRG-KSDD--VP S--KEVVKDMKA-YWEKLYAAGLISQRKFORLTKG--EQGGLTL | 921 |
| WP_016622645 | 853 | IPQSFMKDDSLDNLVLVGSTENRG-KSDD--VP S--KEVVKDMKA-YWEKLYAAGLISQRKFORLTKG--EQGGLTL | 921 |
| WP_033624816 | 853 | IPQSFMKDDSLDNLVLVGSTENRG-KSDD--VP S--KEVVKKMKA-YWEKLYAAGLISQRKFORLTKG--EQGGLTL | 921 |
| WP_033625576 | 853 | IPQSFMKDDSLDNLVLVGSTENRG-KSDD--VP S--KEVVKKMKA-YWEKLYAAGLISQRKFORLTKG--EQGGLTL | 921 |
| WP_033789179 | 853 | IPQSFMKDDSLDNLVLVGSTENRG-KSDD--VP S--KEVVKKMKA-YWEKLYAAGLISQRKFORLTKG--EQGGLTL | 921 |
| WP_002310644 | 853 | IPRSFTTDNSIDNKVLVSSKENRL-KKDD--VP S--EKVVKKMRS-FWYDLYSSKLISKRKLDNLTKIk-----LTE | 918 |
| WP_002310644 | 853 | IPRSFTTDNSIDNKVLVSSKENRL-KKDD--VP S--EKVVKKMRS-FWYDLYSSKLISKRKLDNLTKIk-----LTE | 918 |
| WP_002312694 | 854 | IPRSFTTDNSIDNKVLVSSKENRL-KKDD--VP S--EKVVKKMRS-FWYDLYSSKLISKRKLDNLTKIk-----LTE | 919 |
| WP_002314015 | 854 | IPRSFTTDNSIDNKVLVSSKENRL-KKDD--VP S--EKVVKKMRS-FWYDLYSSKLISKRKLDNLTKIk-----LTE | 919 |
| WP_002320716 | 854 | IPRSFTTDNSIDNKVLVSSKENRL-KKDD--VP S--EKVVKKMRS-FWYDLYSSKLISKRKLDNLTKIk-----LTE | 919 |
| WP_002330729 | 853 | IPRSFTTDNSIDNKVLVSSKENRL-KKDD--VP S--EKVVKKMRS-FWYDLYSSKLISKRKLDNLTKIk-----LTE | 918 |
| WP_002335161 | 854 | IPRSFTTDNSIDNKVLVSSKENRL-KKDD--VP S--EKVVKKMRS-FWYDLYSSKLISKRKLDNLTKIk-----LTE | 919 |
| WP_002345439 | 854 | IPRSFTTDNSIDNKVLVSSKENRL-KKDD--VP S--EKVVKKMRS-FWYDLYSSKLISKRKLDNLTKIk-----LTE | 919 |
| WP_034867970 | 845 | IPRSFIVDNSIDNKVLVASKQNQK-KRDD--VP K--KQIVNEQRI-FWNOLKEAKLISTKKYAYLTKIe-----LTP | 910 |
| WP_047937432 | 854 | IPRSFTTDNSIDNKVLVSSKENRL-KKDD--VP S--EKVVKKMRS-FWYDLYSSKLISKRKLDNLTKIk-----LTE | 919 |
| WP_010720994 | 845 | IPRSFIVDNSIDNKVLVASKQNQK-KKDD--VP K--KQIVNEQRI-FWNOLKEAKLISPKKYAYLTKIe-----LTP | 910 |
| WP_010737004 | 845 | IPRSFIVDNSIDNKVLVSSKENRL-KKDD--VP K--KQIVNEQRI-FWNQLKEAKLISPKKYAYLTKIe-----LTP | 910 |
| WP_034700478 | 845 | IPRSFIVDNSIDNKVLVSSKENRL-KKDD--VP N--KQIVNEQRI-FWNQLKEAKLISPKKYAYLTKIe-----LTP | 910 |
| WP_007209003 | 846 | IPQSFLTDNSIDNRVLTTSKSNRG-KSDN--VP S--EEVVRKMDR-FWRKLLNAKLISERKYTNLTKKe-----LTE | 911 |
| WP_023519017 | 839 | IPRSFIVDNSLDNKVLVSSKVNRG-KLDN--AP D--PLVVKRMRS-HWEKLHQAKLISDKKLANLTKQn-----LTE | 904 |
| WP_010770040 | 846 | VPQSFTTDNSLDNRVLVSSKENRG-KKDD--VP S--KEVVQKNIT-LWETLKNSNLISQKKYDNLTKG--LRGGLTE | 914 |
| WP_048604708 | 843 | IPQSFIVDNSLDNRVLVSSSKNRG-KLDD--VP S--KEVVKKMRA-FWESLYRSGLISKKKFDNLVKA--ESGGLSE | 911 |
| WP_010750235 | 848 | IPRSFIVDHSLDNKVLVSSKENRL-KKDD--VP D--SKVVKRMKA-YWEKLLRANLISERKFSYLTKLe-----LTD | 913 |
| AII16583 | 881 | VPQSFLKDDSIDNKVLTRSDKNRG-KSDN--VP S--EEVVKKMKN-YWRQLLNAKLITQRKEDNLTKA--ERGGLSE | 949 |
| WP_029073316 | 861 | VPQSLLKDDSIDNKVLVLSSENQR-KLDDlvlP ---EMIRNKMFG-FWNKLYENKIISPKKFYSLIKSe-----YSD | 927 |
| WP_031589969 | 861 | VPQSLLKDDSIDNKVLVLSSENQR-KLDDlvlP ---SSIRNKMYG-FWEKLENNKIISPKKFYSLIKTe-----FNE | 927 |
| KDA45870 | 834 | IPQSFLKDDSIENKVLTIKKENVR-KTNG--LP S--EAVIQKMGS-FWKKLLDAGAMTNKKYDNLRRN1--HGGLNE | 902 |
| WP_039099354 | 857 | LPQSFIKDNSLDNRVLVSQRMNRS-KADQ--VP S--VELGQKMQI-QWEQMLRAGLITKKKYDNLTLNp-------- | 923 |
| AKP02966 | 859 | LPRTYIPDDSLENKALVLAKENQR-KADDllLN S---NVIDKNLE-RWTYMLNNNMMGLKKFKNLTRRv-----ITD | 925 |
| WP_010991369 | 845 | VPQSFITDNSIDNLVLTSSAGNRE-KGDD--VP P--LEIVRKRKV-FWEKLYQGNLMSKRKFDYLTKA--ERGGLTE | 913 |
| WP_033838504 | 845 | VPQSFITDNSIDNLVLTSSAGNRE-KGDD--VP P--LEIVRKRKV-FWEKLYQGNLMSKRKFDYLTKA--ERGGLTE | 913 |
| EHN60060 | 848 | VPQSFITDNSIDNLVLTSSAGNRE-KGDD--VP P--LEIVRKRKV-FWEKLYQGNLMSKRKFDYLTKA--ERGGLTE | 916 |
| EFR89594 | 614 | VPQSFITDNSIDNLVLTSSAGNRE-KGDD--VP P--LEIVQKRKV-FWEKLYQGNLMSKRKFDYLTKA--ERGGLTE | 682 |
| WP_038409211 | 845 | IPQSFITDNSIDNRVLTSSTANRE-KGDN--VP L--LEVVRKRKA-FWEKLYQAKLMSKRKFDYLTKA--ERGGLTE | 913 |
| EFR95520 | 464 | IPQSFITDNSIDNRVLTSSTANRE-KGDN--VP L--LEVVRKRKA-FWEKLYQAKLMSKRKEDYLTKA--ERGGLTE | 532 |
| WP_003723650 | 845 | VPQSFITDNSIDNLVLTSSAGNRE-KGGD--VP P--LEIVRKRKV-FWEKLYQGNLMSKRKFDYLTKA--ERGGLTE | 913 |
| WP_003727705 | 845 | VPQSFITDNSIDNLVLTSSAGNRE-KGGD--VP P--LEIVRKRKV-FWEKLYQGNLMSKRKFDYLTKA--ERGGLTE | 913 |
| WP_003730785 | 845 | VPQSFITDNSIDNLVLTSSAGNRE-KGGD--VP P--LEIVRKRKV-FWEKLYQGNLMSKRKFDYLTKA--ERGGLTE | 913 |
| WP_003733029 | 845 | VPQSFITDNSVDNLVLTSSAGNRE-KGGN--VP P--LEIVQKRKI-FWEKLYQGNLMSKRKFDYLTKA--ERGGLTE | 913 |
| WP_003739838 | 845 | VPQSFITDNSIDNLVLTSSAGNRE-KGDD--VP P--LEIVRKRKV-FWEKLFQGNLMSKRKFDYLTKA--ERGGLTE | 913 |
| WP_014601172 | 845 | VPQSFITDNSIDNLVLTSSAGNRE-KGGD--VP P--LEIVRKRKV-FWEKLYQGNLMSKRKFDYLTKA--ERGGLTD | 913 |
| WP_023548323 | 845 | VPQSFITDNSIDNLVLTSSAGNRE-KGDN--VP P--LEIVQKRKI-FWEKLYQGNLMSKRKFDYLTKA--ERGGLTE | 913 |
| WP_031665337 | 845 | VPQSFITDNSIDNLVLTSSAGNRE-KGGD--VP P--LEIVRKRKV-FWEKLYQGNLMSKRKFDYLTKA--ERGGLTE | 913 |
| WP_031669209 | 845 | VPQSFITDNSVDNLVLTSSAGNRE-KGDN--VP P--LEIVOKRKI-FWEKLYQGNLMSKRKFDYLTKA--ERGGLTE | 913 |
| WP_033920898 | 845 | VPQSFITDNSIDNLVLTSSAGNRE-KGDN--VP P--LEIVOKRKI-FWEKLYQGNLMSKRKEDYLTKA--ERGGLTE | 913 |
| AKI42028 | 848 | VPQSFITDNSIDNLVLTSSAGNRE-KGGD--VP P--LEIVRKRKV-FWEKLYQGNLMSKRKFDYLTKA--ERGGLTD | 916 |
| AKI50529 | 848 | VPQSFITDNSIDNLVLTSSAGNRE-KGDN--VP P--LEIVQKRKI-FWEKLYQGNLMSKRKFDYLTKA--ERGGLTE | 916 |
| EFR83390 | 293 | VPQSFITDNSIDNLVLTSSAGNRE-KGDD--VP P--LEIVRKRKV-FWEKLYQGNLMSKRKFDYLTKA--ERGGLTE | 361 |
| WP_046323366 | 845 | VPQSFITDNSIDNRVLASSAANRE-KGDN--VP S--LEVVRKRKV-YWEKLYQAKLMSKRKEDYLTKA--ERGGLTE | 913 |
| AKE81011 | 858 | VPQSFLKDDSIDNKVLTRSDKNRG-KSDN--VP S--EEVVKKMKN-YWROLLNAKLITORKFDNLTKA--ERGGLSE | 926 |
| CUO82355 | 854 | VPQSLVKDDSFDNRVLVLPSENQR-KLDD1vVP ---FDIRDKMYR-FWKLLFDHELISPKKFYSLIKTe-----YTE | 920 |
| WP_033162887 | 856 | LPQSLIKDDSFDNRVLVLPEENQW-KLDSetVP ---FEIRNKMIG-FWQMLHENGLMSNKKFFSLIRTd-----FSD | 922 |
| AGZ01981 | 875 | VPQSFLKDDSIDNKVLTRSDKNRG-KSDN--VP S--EEVVKKMKN-YWROLLNAKLITORKFDNLTKA--ERGGLSE | 943 |
| AKA60242 | 842 | VPQSFLKDDSIDNKVLTRSDKNRG-KSDN--VP S--EEVVKKMKN-YWRQLLNAKLITORKFDNLTKA--ERGGLSE | 910 |
| AKS40380 | 842 | VPQSFLKDDSIDNKVLTRSDKNRG-KSDN--VP S--EEVVKKMKN-YWRQLLNAKLITQRKFDNLTKA--ERGGLSE | 910 |
| 4UN5_B | 846 | VPQSFLKDDSIDNKVLTRSDKNRG-KSDN--VP S--EEVVKKMKN-YWROLLNAKLITORKFDNLTKA--ERGGLSE | 914 |
| WP_010922251 | 911 | 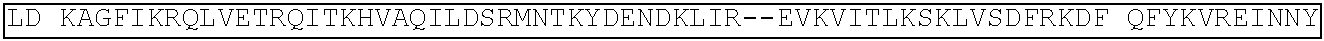 | 981 |
| WP_039695303 | 921 | AD KAGFIKRQLVETROITKHVAQILDARFNTEHDENDKVIR--DVKVITLKSNLVSQFRKDF EFYKVREINDY | 991 |
| WP_045635197 | 913 | RD KVGFIKRQLVETRQITKHVAQILDARYNTEVNEKDKKNR--TVKIITLKSNLVSNFRKEF RLYKVREINDY | 983 |
| 5AXW_A | 633 | RD QKDFINRNLVDTRYATRGLMNLLRSYFR---------VNn1DVKVKSINGGFTSFLRRKW KFKKERNKGYK | 702 |
| WP_009880683 | 595 | LD KAGFIKRQLVETROITKHVAQILDSRMNTKYDENDKLIR--EVRVITLKSKLVSDFRKDF QFYKVREINNY | 665 |
| WP_010922251 | 911 | LD KAGFIKRQLVETROITKHVAQILDSRMNTKYDENDKLIR--EVKVITLKSKLVSDFRKDF QFYKVREINNY | 981 |
| WP_011054416 | 911 | LD KVGFIKRQLVETROITKHVAQILDSRMNTKYDENDKLIR--EVRVITLKSKLVSDFRKDF QFYKVREINNY | 981 |
| WP_011284745 | 911 | LD KAGFIKRQLVETROITKHVAQILDSRMNTKYDENDKLIR--EVKVITLKSKLVSDFRKDF QFYKVREINNY | 981 |
| WP_011285506 | 911 | LD KAGFIKRQLVETROITKHVAQILDSRMNTKYDENDKLIR--EVKVITLKSKLVSDFRKDF QFYKVREINNY | 981 |
| WP_011527619 | 911 | LD KAGFIKRQLVETRQITKHVAQILDSRMNTKYDENDKLIR--EVKVITLKSKLVSDFRKDF QFYKVREINNY | 981 |
| WP_012560673 | 911 | LD KAGFIKRQLVETRQITKHVAQILDSRMNTKYDENDKLIR--EVRVITLKSKLVSDFRKDF QFYKVREINNY | 981 |
| WP_014407541 | 910 | LD KAGFIKRQLVETRQITKHVAQILDSRMNTKYDENDKLIR--EVKVITLKSKLVSDFRKDF QFYKVREINNY | 980 |
| WP_020905136 | 911 | LD KAGFIKRQLVETROITKHVAQILDSRMNTKYDENDKLIR--EVKVITLKSKLVSDFRKDF QFYKVREINNY | 981 |
| WP_023080005 | 910 | LD KAGFIKRQLVETRQITKHVAQILDSRMNTKYDENDKLIR--EVKVITLKSKLVSDFRKDF QFYKVREINNY | 980 |
| WP_023610282 | 910 | LD KAGFIKRQLVETROITKHVAQILDSRMNTKYDENDKLIR--EVKVITLKSKLVSDFRKDF QFYKVREINNY | 980 |
| WP_030125963 | 911 | LD KAGFIKRQLVETROITKHVAQILDSRMNTKYDENDKLIR--EVKVITLKSKLVSDFRKDF QFYKVREINNY | 981 |
| WP_030126706 | 911 | LD KAGFIKRQLVETROITKHVAQILDSRMNTKYDENDKLIR--EVKVITLKSKLVSDFRKDF QFYKVREINNY | 981 |
| WP_031488318 | 911 | LD KAGFIKRQLVETRQITKHVAQILDSRMNTKYDENDKLIR--EVKVITLKSKLVSDERKDF QFYKVREINNY | 981 |
| WP_032460140 | 911 | LD KAGFIKRQLVETRQITKHVAQILDSRMNTKYDENDKLIR--EVRVITLKSKLVSDFRKDF QFYKVREINNY | 981 |
| WP_032461047 | 911 | LD KAGFIKRQLVETROITKHVAQILDSRMNTKYDENDKLIR--EVRVITLKSKLVSDFRKDF QFYKVREINNY | 981 |
| WP_032462016 | 911 | LD KAGFIKRQLVETROITKHVAQILDSRMNTKYDENDKLIR--EVKVITLKSKLVSDERKDF QFYKVREINNY | 981 |
| WP_032462936 | 911 | LD KAGFIKRQLVETROITKHVAQILDSRMNTKYDENDKLIR--EVRVITLKSKLVSDERKDE QFYKVREINNY | 981 |
| WP_032464890 | 911 | LD KAGFIKRQLVETROITKHVAQILDSRMNTKYDENDKLIR--EVKVITLKSKLVSDFRKDF QFYKVREINNY | 981 |
| WP_033888930 | 736 | LD KAGFIKRQLVETROITKHVAQILDSRMNTKYDENDKLIR--EVKVITLKSKLVSDFRKDF QFYKVREINNY | 806 |
| WP_038431314 | 911 | LD KAGFIKRQLVETROITKHVAQILDSRMNTKYDENDKLIR--EVKVITLKSKLVSDERKDF QFYKVREINNY | 981 |
| WP_038432938 | 910 | LD KAGFIKRQLVETROITKHVAQILDSRMNTKYDENDKLIR--EVKVITLKSKLVSDFRKDF QFYKVREINNY | 980 |
| WP_038434062 | 911 | LD KAGFIKRQLVETROITKHVAQILDSRMNTKYDENDKLIR--EVKVITLKSKLVSDERKDF QFYKVREINNY | 981 |
| BAQ51233 | 822 | LD KAGFIKRQLVETROITKHVAQILDSRMNTKYDENDKLIR--EVKVITLKSKLVSDFRKDF QFYKVREINNY | 892 |
| KGE60162 | 86 | LD KVGFIKRQLVETROITKHVAQILDSRMNTKYDENDKLIR--EVRVITLKSKLVSDFRKDF QFYKVREINNY | 156 |
| KGE60856 | | -- ------------------------------------------------------------ ----------- | |
| WP_002989955 | 911 | LD KAGFIKRQLVETROITKHVAQILDSRMNTKYDENDKLIR--EVKVITLKSKLVSDERKDF QFYKVREINNY | 981 |
| WP_003030002 | 910 | ED KAGFIKRQLVETRQITKHVAQILDERENTEFDGNKRRIR--NVKIITLKSNLVSNERKEF ELYKVREINDY | 980 |
| WP_003065552 | 921 | AD KAGFIKRQLVETROITKHVAQILDARFNTESDENDKVIR--DVKVITLKSNLVSQFRKDF EFYKVREINDY | 991 |
| WP_001040076 | 916 | DD KARFIQRQLVETRQITKHVARILDERFNNELDSKGRRIR--KVKIVTLKSNLVSNERKEF VFYKIREVNNY | 986 |
| WP_001040078 | 916 | DD KARFIQROLVETROITKHVARILDERFNNELDSKGRRIR--KVKIVTLKSNLVSNERKEF GFYKIREVNDY | 986 |
| WP_001040080 | 916 | DD KARFIQRQLVETRQITKHVARILDERFNNELDSKGRRIR--KVKIVTLKSNLVSNERKEF GFYKIREVNNY | 986 |
| WP_001040081 | 916 | DD KARFIQRQLVETRQITKHVARILDERFNNELDSKGRRIR--KVKIVTLKSNLVSNERKEF GFYKIREVNNY | 986 |
| WP_001040083 | 916 | DD KARFIQROLVETROITKHVARILDERFNNELDSKGRRIR--KVKIVTLKSNLVSNERKEF GFYKIREVNNY | 986 |
| WP_001040085 | 916 | DD KARFIQROLVETRQITKHVARILDERFNNELDSKGRRIR--KVKIVTLKSNLVSNERKEF GFYKIREVNNY | 986 |
| WP_001040087 | 916 | DD KARFIQRQLVETRQITKHVARILDERFNNELDSKGRRIR--KVKIVTLKSNLVSNERKEF GFYKIREVNNY | 986 |
| WP_001040088 | 916 | DD KARFIQROLVETROITKHVARILDERFNNELDSKGRRIR--KVKIVTLKSNLVSNFRKEF GFYKIREVNNY | 986 |
| WP_001040089 | 916 | DD KARFIQROLVETRQITKHVARILDERFNNELDSKGRRIR--KVKIVTLKSNLVSNERKEF GFYKIREVNNY | 986 |
| WP_001040090 | 916 | DD KARFIQRQLVETRQITKHVARILDERFNNELDSKGRRIR--KVKIVTLKSNLVSNFRKEF GFYKIREVNNY | 986 |
| WP_001040091 | 916 | DD KARFIQRQLVETROITKHVARILDERFNNELDSKGRRIR--KVKIVTLKSNLVSNERKEF GFYKIREVNNY | 986 |
| WP_001040092 | 916 | DD KAGFIQRQLVETROITKHVARILDERFNNKVDDNNKPIR--KVKIVTLKSNLVSNFRKEF GFYKIREVNNY | 986 |
| WP_001040094 | 916 | DD KARFIQROLVETROITKHVARILDERFNNELDSKGRRIR--KVKIVTLKSNLVSNERKEF GFYKIREVNNY | 986 |
| WP_001040095 | 916 | DD KARFIQROLVETRQITKHVARILDERFNNELDSKGRRIR--KVKIVTLKSNLVSNERKEF GFYKIREVNNY | 986 |
| WP_001040096 | 916 | DD KARFIQROLVETROITKHVARILDERFNNELDSKGRRIR--KVKIVTLKSNLVSNERKEF GFYKIREVNNY | 986 |
| WP_001040097 | 916 | DD KARFIQROLVETROITKHVARILDERFNNELDSKGRRIR--KVKIVTLKSNLVSNERKEF GFYKIREVNNY | 986 |
| WP_001040098 | 916 | DD KARFIQROLVETROITKHVARILDERFNNELDSKGRRIR--KVKIVTLKSNLVSNERKEF GFYKIREVNNY | 986 |
| WP_001040099 | 916 | DD KARFIQROLVETROITKHVARILDERFNNELDSKGRRIR--KVKIVTLKSNLVSNERKEF GFYKIREVNNY | 986 |
| WP_001040100 | 916 | DD KARFIQRQLVETRQITKHVARILDERFNNELDSKGRRIR--KVKIVTLKSNLVSNERKEF GFYKIREVNNY | 986 |
| WP_001040104 | 916 | DD KARFIQRQLVETRQITKHVARILDERFNNELDSKGRRIR--KVKIVTLKSNLVSNFRKEF GFYKIREVNNY | 986 |
| WP_001040105 | 916 | DD KARFIQROLVETROITKHVARILDERFNNELDSKGRRIR--KVKIVTLKSNLVSNERKEF GFYKIREVNNY | 986 |
| WP_001040106 | 916 | DD KARFIQROLVETROITKHVARILDERFNNELDSKGRRIR--KVKIVTLKSNLVSNFRKEF GFYKIREVNDY | 986 |
| WP_001040107 | 916 | DD KARFIQROLVETROITKHVARILDERFNNELDSKGRRIR--KVKIVTLKSNLVSNFRKEF GFYKIREVNDY | 986 |
| WP_001040108 | 916 | DD KARFIQROLVETRQITKHVARILDERFNNELDSKGRRIR--KVKIVTLKSNLVSNERKEF GFYKIREVNDY | 986 |
| WP_001040109 | 916 | DD KARFIQRQLVETROITKHVARILDERFNNELDSKGRRIR--KVKIVTLKSNLVSNERKEF GFYKIREVNDY | 986 |
| WP_001040110 | 916 | DD KARFIQROLVETROITKHVARILDERFNNELDSKGRRIR--KVKIVTLKSNLVSNERKEF GFYKIREVNDY | 986 |
| WP_015058523 | 916 | DD KAGFIQRQLVETROITKHVARILDERFNNKVDDNNKPIR--KVKIVTLKSNLVSNERKEF GFYKIREVNNY | 986 |
| WP_017643650 | 916 | DD KARFIQROLVETROITKHVARILDERFNNELDSKGRRIR--KVKIVTLKSNLVSNERKEF GFYKIREVNNY | 986 |
| WP_017647151 | 916 | DD KARFIQROLVETROITKHVARILDERFNNELDSKGRRIR--KVKIVTLKSNLVSNERKEF GFYKIREVNNY | 986 |
| WP_017648376 | 916 | DD KARFIQRQLVETRQITKHVARILDERFNNELDSKGRRIR--KVKIVTLKSNLVSNERKEF GFYKIREVNNY | 986 |
| WP_017649527 | 916 | DD KARFIQROLVETROITKHVARILDERFNNELDSKGRRIR--KVKIVTLKSNLVSNERKEF GFYKIREVNNY | 986 |
| WP_017771611 | 916 | DD KARFIQRQLVETROITKHVARILDERFNNELDSKGRRIR--KVKIVTLKSNLVSNERKEF GFYKIREVNDY | 986 |
| WP_017771984 | 916 | DD KARFIQRQLVETROITKHVARILDERFNNELDSKGRRIR--KVKIVTLKSNLVSNERKEF GFYKIREVNNY | 986 |
| CFQ25032 | 916 | DD KARFIQRQLVETROITKHVARILDERFNNELDSKGRRIR--KVKIVTLKSNLVSNFRKEF GFYKIREVNNY | 986 |
| CFV16040 | 916 | DD KARFIQRQLVEIRQITKHVARILDERFNNELDSKGRRIR--KVKIVTLKSNLVSNFRKEF GFYKIREVNNY | 986 |
| KLJ37842 | 916 | DD KARFIQRQLVETRQITKHVARILDERFNNELDSKGRRIR--KVKIVTVKSNLVSNERKEF GFYKIREVNNY | 986 |
| KLJ72361 | 916 | DD KARFIQRQLVETRQITKHVARILDELFNNELDSKGRRIR--KVKIVTLKSNLVSNFRKEF GFYKIREVNNY | 986 |
| KLL20707 | 930 | DD KARFIQRQLVETRQITKHVARILDERFNNELDSKGRRIR--KVKIVTLKSNLVSNFRKEF GFYKIREVNNY | 1000 |
| KLL42645 | 916 | DD KARFIQRQLVETROITKHVARILDERFNNELDSKGRRIR--KVKIVTLKSNLVSNERKEF GFYKIREVNDY | 986 |
| WP_047207273 | 916 | DD KARFIQRQLVETROITKHVARILDERFNNELDSKGRRIR--KVKIVTLKSNLVSNERKEF GFYKIREVNNY | 986 |
| WP_047209694 | 916 | DD KARFIQRQLVETROITKHVARILDERFNNELDSKGRRIR--KVKIVTLKSNLVSNFRKEF GFYKIREVNNY | 986 |
| WP_050198062 | 916 | DD KARFIQRQLVETRQITKHVARILDERFNNELDSKGRRIR--KVKIVTLKSNLVSNFRKEF GFYKIREVNNY | 986 |
| WP_050201642 | 916 | DD KARFIQROLVETROITKHVASILDERFNNELDSKGRRIR--KVKIVTLKSNLVSNERKEF GFYKIREVNNY | 986 |
| WP_050204027 | 916 | DD KARFIQRQLVETROITKHVARILDERFNNELDSKGRRIR--KVKIVTLKSNLVSNERKEF GFYKIREVNDY | 986 |
| WP_050881965 | 916 | DD KARFIQRQLVETRQITKHVARILDERFNNELDSKGRRIR--KVKIVILKSNLVSNERKEF GFYKIREVNNY | 986 |
| WP_050886065 | 916 | DD KARFIQROLVETROITKHVARILDERFNNELDSKGRRIR--KVKIVTLKSNLVSNERKEF GFYKIREVNNY | 986 |
| AHN30376 | 916 | DD KAGFIQRQLVETROITKHVARILDERFNNKVDDNNKPIR--KVKIVTLKSNLVSNERKEF GFYKIREVNNY | 986 |
| EAO78426 | 916 | DD KARFIQRQLVETROITKHVARILDERFNNELDSKGRRIR--KVKIVTLKSNLVSNFRKEF GFYKIREVNNY | 986 |
| CCW42055 | 916 | DD KARFIQRQLVETROITKHVARILDERFNNELDSKGRRIR--KVKIVTLKSNLVSNFRKEF GFYKIREVNDY | 986 |
| WP_003041502 | 910 | ND KAGFIKRQLVETROITKHVAQVLDARFNAKHDENKKVIR--DVKIITLKSNLVSQFRKDF KFYKVREINDY | 980 |
| WP_037593752 | 911 | ED KAGFIKRQLVETRQITKHVAQILDERENTEFDGAQRRIR--NVKIITLKSNLVSNERKEF ELYKVREINDY | 981 |
| WP_049516684 | 911 | ED KAGFIKRQLVETRQITKHVAQILDERENTEFDGAQRRIR--NVKIITLKSNLVSNERKEF ELYKVREINDY | 981 |
| GAD46167 | 910 | ED KAGFIKRQLVETRQITKHVAQILDERENTEFDGAQRRIR--NVKIITLKSNLVSNFRKEF ELYKVREINDY | 980 |
| WP_018363470 | 919 | AD KAGFIKRQLVETROITKHVAQILDARFNTERDENDKVIR--DVKVITLKSNLVSQFRKEF KFYKVREINDY | 989 |
| WP_003043819 | 920 | AD KAGFIKRQLVETROITKHVARILDSRMNTKRDKNDKPIR--EVKVITLKSKLVSDFRKDF QLYKVRDINNY | 990 |
| WP_006269658 | 910 | ED KAGFIKRQLVETROITKHVAQILDERFNTEFDGNKRRIR--NVKIITLKSNLVSNFRKEF ELYKVREINDY | 980 |
| WP_048800889 | 910 | ND KAGFIHRQLVETROITKHVAQILDARFNPKRDDNKKVIR--DVKIITLKSNLVSQFRRDF KLYKVREINDY | 980 |
| WP_012767106 | 910 | LD KAGFIKRQLVETROITKHVAQILDSRMNTKYDENDKLIR--EVKVITLKSKLVSDFRKDF QFYKVREINNY | 980 |
| WP_014612333 | 910 | LD KAGFIKRQLVETRQITKHVAQILDSRMNTKYDENDKLIR--EVKVITLKSKLVSDERKDF QFYKVREINNY | 980 |
| WP_015017095 | 910 | LD KAGFIKRQLVETRQITKHVAQILDSRMNTKYDENDKLIR--EVKVITLKSKLVSDERKDE QFYKVREINNY | 980 |
| WP_015057649 | 910 | LD KAGFIKRQLVETRQITKHVAQILDSRMNTKYDENDKLIR--EVKVITLKSKLVSDFRKDF QFYKVREINNY | 980 |
| WP_048327215 | 910 | LD KAGFIKRQLVETRQITKHVAQILDSRMNTKYDENDKLIR--EVKVITLKSKLVSDFRKDF QFYKVREINNY | 980 |
| WP_049519324 | 910 | LD KAGFIKRQLVETROITKHVAQILDSRMNTKYDENDKLIR--EVKVITLKSKLVSDFRKDF QFYKVREINNY | 980 |
| WP_012515931 | 910 | VD KAGFIQROLVETROITKHVAQILDSRENTEFDDHNKRIR--KVHIITLKSKLVSDFRKEF GLYKIRDINHY | 980 |
| WP_021320964 | 910 | VD KAGFIQROLVETROITKHVAQILDSRENTEFDDHNKRIR--KVHIITLKSKLVSDFRKEF GLYKIRDINHY | 980 |
| WP_037581760 | 910 | VD KAGFIQLOLVETROITKHVAQILDSRENTEFDDHNKRIR--KVHIITLKSKLVSDFRKEF GLYKIRDINHY | 980 |
| WP_004232481 | 918 | TD KAGFIKRQLVETROITKHVAQILDARFNTKCDENDKVIR--DVKVITLKSSLVSQFRKEF KFYKVREINDY | 988 |
| WP_009854540 | 919 | AD KAGFIKRQLVETROITKHVAQILDARFNTEHDENDKVIR--DVKVITLKSNLVSQFRKDF EFYKVREINDY | 989 |
| WP_012962174 | 919 | ND KAGFIKRQLVETROITKHVAQILDSRENTERDENDKVIR--NVKVITLKSNLVSQFRKDF KFYKVREINDY | 989 |
| WP_039695303 | 921 | AD KAGFIKRQLVETROITKHVAQILDARFNTEHDENDKVIR--DVKVITLKSNLVSQFRKDF EFYKVREINDY | 991 |
| WP_014334983 | 918 | AD KAGFIKRQLVETROITKHVAQILDARFNTKRDENDKVIR--DVKVITLKSNLVSQFRKEF KFYKVREINDY | 988 |
| WP_003099269 | 911 | FD KAGFIKRQLVETROITKHVAQILDSRFNSNLTEDSKSNR--NVKIITLKSKMVSDERKDF GFYKLREVNDY | 981 |
| AHY15608 | 911 | FD KAGFIKRQLVETROITKHVAQILDSRENSNLTEDSKSNR--NVKIITLKSKMVSDERKDF GFYKLREVNDY | 981 |
| AHY17476 | 911 | FD KAGFIKRQLVETROITKHVAQILDSRENSNLTEDSKSNR--NVKIITLKSKMVSDERKDF GFYKLREVNDY | 981 |
| ESR09100 | | -- ------------------------------------------------------------ ----------- | |
| AGM98575 | 911 | FD KAGFIKRQLVETROITKHVAQILDSRENSNLTEDSKSNR--NVKIITLKSKMVSDERKDF GFYKLREVNDY | 981 |
| ALF27331 | 911 | DD KAGFIKRQLVETROITKHVARILDERENTETDENNKKIR--QVKIVTLKSNLVSNFRKEF ELYKVREINDY | 981 |
| WP_018372492 | 924 | ED KAGFIRRQLVETROITKHVARLLDEKLNRKKNENGEKLR--TTKIITLKSVFASRFRANF DLYKLRELNHY | 994 |
| WP_045618028 | 914 | RD KVGFIKRQLVETRQITKHVAQILDARFNTEVTEKDKKDR--SVKIITLKSNLVSNFRKEF RLYKVREINDY | 984 |
| WP_045635197 | 913 | RD KVGFIKRQLVETROITKHVAQILDARYNTEVNEKDKKNR--TVKIITLKSNLVSNERKEF RLYKVREINDY | 983 |
| WP_002263549 | 911 | DD KAGFIKRQLVETROITKHVARILDERENTETDENNKKIR--QVKIVTLKSNLVSNERKEF ELYKVREINDY | 981 |
| WP_002263887 | 911 | DD KAGFIKRQLVETROITKHVARILDERENTETDENNKKIR--QVKIVTLKSNLVSNFRKEF ELYKVREINDY | 981 |
| WP_002264920 | 911 | DD KAGFIKRQLVETRQITKHVARILDERENTETDENNKKIR--QVKIVILKSNLVSNERKEF ELYKVREINDY | 981 |
| WP_002269043 | 911 | DD KAGFIKRQLVETROITKHVARILDERENTETDENNKKIR--QVKIVILKSNLVSNFRKEF ELYKVREINDY | 981 |
| WP_002269448 | 911 | DD KAGFIKRQLVETROITKHVARILDERFYTETDENNKKIR--QVKIVTLKSNLVSNERKEF ELYKVREINDY | 981 |
| WP_002271977 | 911 | DD KAGFIKRQLVETROITKHVARILDERFYTETDENNKKIR--QVKIVTLKSNLVSNERKEF ELYKVREINDY | 981 |
| WP_002272766 | 911 | DD KAGFIKRQLVETROITKHVARILDERENTETDENNKKIR--QVKIVTLKSNLVSNERKEF ELYKVREINDY | 981 |
| WP_002273241 | 911 | DD KAGFIKRQLVETRQITKHVARILDERFYTETDENNKKIR--QVKIVILKSNLVSNERKEF ELYKVREINDY | 981 |
| WP_002275430 | 911 | DD KAGFIKRQLVETROITKHVARILDERENTETDENNKKIR--QVKIVTLKSNLVSNERKEF ELYKVREINDY | 981 |
| WP_002276448 | 911 | DD KAGFIKROLVETRQITKHVARILDERFYTETDENNKKIR--QVKIVTLKSNLVSNFRKEF ELYKVREINDY | 981 |
| WP_002277050 | 913 | DD KAGFIKRQLVETROITKHVARMLDERENKEFDDNNKRIR--RVKIVTLKSNLVSSFRKEF ELYKVREINDY | 983 |
| WP_002277364 | 911 | DD KAGFIKRQLVETROITKHVARILDERFHTETDENNKKIR--QVKIVTLKSNLVSNERKEF ELYKVREINDY | 981 |
| WP_002279025 | 911 | DD KAGFIKRQLVETROITKHVARILDERENTETDENNKKIR--QVKIVTLKSNLVSNFRKEF ELYKVREINDY | 981 |
| WP_002279859 | 911 | DD KAGFIKRQLVETRQITKHVARILDERENTETDENNKKIR--QVKIVTLKSNLVSNFRKEF ELYKVREINDY | 981 |
| WP_002280230 | 911 | DD KAGFIKRQLVETRQITKHVARILDERFYTETDENNKKIR--QVKIVTLKSNLVSNERKEF ELYKVREINDY | 981 |
| WP_002281696 | 911 | DD KAGFIKRQLVETROITKHVARILDERFHTETDENNKKIR--QVKIVTLKSNLVSNERKEF ELYKVREINDY | 981 |
| WP_002282247 | 913 | DD KAGFIKRQLVETROITKHVARMLDERENKEFDDNNKRIR--RVKIVTLKSNLVSSFRKEF ELYKVREINDY | 983 |
| WP_002282906 | 911 | DD KAGFIKRQLVETROITKHVARILDERENTETDENNKKIR--QVKIVILKSNLVSNERKEF ELYKVREINDY | 981 |
| WP_002283846 | 911 | DD KAGFIKRQLVETRQITKHVARILDERFHTETDENNKKIR--QVKIVTLKSNLVSNERKEF ELYKVREINDY | 981 |
| WP_002287255 | 911 | DD KAGFIKRQLVETROITKHVARILDERFHTETDENNKKIR--QVKIVTLKSNLVSNFRKEF ELYKVREINDY | 981 |
| WP_002288990 | 911 | DD KAGFIKRQLVETROITKHVARILDERENTETDENNKKIR--QVKIVILKSNLVSNFRKEF ELYKVREINDY | 981 |
| WP_002289641 | 911 | DD KAGFIKRQLVETROITKHVARILDERENTETDENNKKIR--QVKIVTLKSNLVSNERKEF ELYKVREINDY | 981 |
| WP_002290427 | 911 | DD KAGFIKRQLVETROITKHVARILDERENTETDENNKKIR--QVKIVTLKSNLVSNERKEF ELYKVREINDY | 981 |
| WP_002295753 | 911 | DD KAGFIKRQLVETRQITKHVARILDERFYTETDENNKKIR--QVKIVTLKSNLVSNERKEF ELYKVREINDY | 981 |
| WP_002296423 | 911 | DD KAGFIKRQLVETROITKHVARILDERFHTETDENNKKIR--QVKIVTLKSNLVSNERKEF ELYKVREINDY | 981 |
| WP_002304487 | 925 | DD KAGFIKRQLVETROITKHVARILDERENTETDENNKKIR--QVKIVTLKSNLVSNERKEF ELYKVREINDY | 995 |
| WP_002305844 | 911 | DD KAGFIKRQLVETROITKHVARILDERFYTETDENNKKIR--QVKIVTLKSNLVSNERKEF ELYKVREINDY | 981 |
| WP_002307203 | 911 | DD KAGFIKRQLVETRQITKHVARILDERFNTETDENNKKIR--QVKIVTLKSNLVSNERKEF ELYKVREINDY | 981 |
| WP_002310390 | 911 | DD KAGFIKHQLVETROITKHVARILDERENTETDENNKKIR--QVKIVTLKSNLVSNFRKEF ELYKVREINDY | 981 |
| WP_002352408 | 911 | DD KAGFIKRQLVETROITKHVARILDERFNTETDENNKKIR--QVKIVTLKSNLVSNERKEF ELYKVREINDY | 981 |
| WP_012997688 | 911 | DD KAGFIKROLVETRQITKHVARILDERFHTETDENNKKIR--QVKIVTLKSNLVSNFRKEF ELYKVREINDY | 981 |
| WP_014677909 | 911 | DD KAGFIKROLVETRQITKHVARILDERFYTETDENNKKIR--QVKIVTLKSNLVSNFRKEF ELYKVREINDY | 981 |
| WP_019312892 | 911 | DD KAGFIKRQLVETROITKHVARILDERFHTETDENNKKIR--QVKIVTLKSNLVSNERKEF ELYKVREINDY | 981 |
| WP_019313659 | 911 | DD KAGFIKROLVETROITKHVARILDERFHTETDENNKKIR--QVKIVTLKSNLVSNERKEF ELYKVREINDY | 981 |
| WP_019314093 | 911 | DD KAGFIKRQLVETRQITKHVARILDERENTETDENNKKIR--QVKIVTLKSNLVSNFRKEF ELYKVREINDY | 981 |
| WP_019315370 | 911 | DD KAGFIKRQLVETROITKHVARILDERFNTETDENNKKIR--QVKIVILKSNLVSNFRKEF ELYKVREINDY | 981 |
| WP_019803776 | 911 | DD KAGFIKRQLVETROITKHVARILDERFNTETDENNKKIR--QVKIVTLKSNLVSNFRKEF ELYKVREINDY | 981 |
| WP_019805234 | 911 | DD KAGFIKROLVETRQITKHVARILDERFNTETDENNKKIR--QVKIVTLKSNLVSNFRKEF ELYKVREINDY | 981 |
| WP_024783594 | 911 | DD KAGFIKRQLVETROITKHVARILDERFNTETDENNKKIR--QVKIVTLKSNLVSNFRKEF ELYKVREINDY | 981 |
| WP_024784288 | 913 | DD KAGFIKRQLVETRQITKHVARMLDERFNKEFDDNNKRIR--RVKIVTLKSNLVSSFRKEF ELYKVREINDY | 983 |
| WP_024784666 | 911 | DD KAGFIKRQLVETRQITKHVARILDERFHTETDENNKKIR--QVKIVTLKSNLVSNERKEF ELYKVREINDY | 981 |
| WP_024784894 | 911 | DD KAGFIKROLVETROITKHVARILDERENTETDENNKKIR--QVKIVTLKSNLVSNERKEF ELYKVREINDY | 981 |
| WP_024786433 | 913 | DD KAGFIKRQLVETROITKHVARMLDERFNKEFDDNNKRIR--RVKIVTLKSNLVSSFRKEF ELYKVREINDY | 983 |
| WP_049473442 | 911 | DD KAGFIKRQLVETROITKHVARILDERENTETDENNKKIR--QVKIVTLKSNLVSNERKEF ELYKVREINDY | 981 |
| WP_049474547 | 911 | DD KAGFIKROLVETRQITKHVARILDERFNTETDENNKKIR--QVKIVTLKSNLVSNERKEF ELYKVREINDY | 981 |
| EMC03581 | 904 | DD KAGFIKRQLVETROITKHVARILDERFHTETDENNKKIR--QVKIVTLKSNLVSNFRKEF ELYKVREINDY | 974 |
| WP_000428612 | 916 | RD KVGFIKRQLVETRQITKHVAQILDARYNTEVNEKDKKNR--TVKIITLKSNLVSNERKEF RLYKIREINDY | 986 |
| WP_000428613 | 914 | RD KVGFIKRQLVETRQITKHVAQILDARFNKEVNEKDKKNR--TVKIITLKSNLVSNFRKEF RLYKVREINDY | 984 |
| WP_049523028 | 909 | RD KVGFIKRQLVETROITKHVAQILDDRFNAEVNEKNQKLR--SVKIITLKSNLVSNFRKEF GLYKVREINDY | 979 |
| WP_003107102 | 880 | YD KAGFIKRQLVETROITKHVAQILNNRENNNVDDSSKNKR--PVKIITLKSKMVSDFRKEF GFYKIREVNDY | 950 |
| WP_054279288 | 912 | SD KANFIQROLVETROITKHVAQILDSRENTERDEKDRPIR--RVKVITLKSKFVSDFRQDE GFYKLREINDY | 982 |
| WP_049531101 | 914 | RD KVGFIKRQLVETRQITKHVAQILDSRFNTKVNEKNQKIR--TVKIITLKSNLVSNFRKEF RLYKVREINDY | 984 |
| WP_049538452 | 914 | RD KVGFIRROLVETRQITKHVAQILDSRFNTEVTEKDKKNR--NVKIITLKSNLVSNFRKEF GLYKVREINDY | 984 |
| WP_049549711 | 916 | LD KVGFIKRQLVETROITKHVAQILDARFNKEVTEKDKKNR--NVKIITLKSNLVSNFRKEF RLYKVREINDY | 986 |
| WP_007896501 | 917 | SD KARFLRRQLVETROITKHVAQLLDSRENSKSNONKKLAR--NVKIITLKSKIVSDFRKDF GLYKLREVNNY | 987 |
| EFR44625 | 869 | SD KARFLRROLVETROITKHVAQLLDSRENSKSNONKKLAR--NVKIITLKSKIVSDFRKDF GLYKLREVNNY | 939 |
| WP_002897477 | 913 | RD KVGFIRROLVETQQITKNVAQILDARFNTEVKEKNQKIR--TVKIITLKSNLVSNERKEF GLYKVREINNY | 983 |
| WP_002906454 | 913 | RD KVGFIKRQLVETRQITKHVAQLLDTRENTEVNEENQKIR--TVKIITLKSNLVSNERKEF GLYKVREINDY | 983 |
| WP_009729476 | 914 | LD KVGFIKRQLVETRQITKHVAQILDARFNKEVTEKDKKNR--TVKIITLKSNLVSNFRKEF ELYKVREINDY | 984 |
| CQR24647 | 913 | ED KAGFIKRQLVETROITKHVARILDERFNRDEDKNDKRIR--NVKIVTLKSNLVSNERKEF GFYKVREINNF | 983 |
| WP_000066813 | 918 | LD KVGFIKRQLVETRQITKHVAQFLDARFNKEVTEKDKKNR--NVKIITLKSNLVSNFRKEF GLYKVREINDY | 988 |
| WP_009754323 | 914 | RD KVGFIKRQLVETRQITKHVARILDARFNTEVSEKNQKIR--SVKIITLKSNLVSNERKEF KLYKVREINDY | 984 |
| WP_044674937 | 913 | ED KARFIQRQLVETROITKHVARILDTRENTKLDEAGNRIRdpKVNIITLKSNLVSQFRKDY QLYKVREINNY | 985 |
| WP_044676715 | 915 | ED KARFIQRQLVETROITKHVARILDTRENTKLDEAGNRIRdpKVNIITLKSNLVSQFRKDY QLYKVREINNY | 987 |
| WP_044680361 | 915 | ED KARFIQRQLVETROITKHVARILDTRENTKLDEAGNRIRdpKVNIITLKSNLVSQFRKDY QLYKVREINNY | 987 |
| WP_044681799 | 913 | ED KARFIQRQLVETROITKHVARILDTRENTKLDEAGNRIRdpKVNIITLKSNLVSQFRKDY QLYKVREINNY | 985 |
| WP_049533112 | 910 | ND KAGFIKRQLVETRQITKHVAQVLDARFNAKHDENKKVIR--DVKIITLKSNLVSQFRKDF KFYKVREINDY | 980 |
| WP_029090905 | 891 | RD KEGFIARQLVETRQITKHVTQLLQQEY-----------K-dTTKVFAIKATLVSGLRRKF EFIKNRNVNDY | 951 |
| WP_006506696 | 917 | RD EERFINROLVETRQITKNVTQIIEDHYST-------------TKVAAIRANLSHEFRVKN HIYKNRDINDY | 976 |
| AIT42264 | 911 | LD KAGFIKRQLVETRQITKHVAQILDSRMNTKYDENDKLIR--EVKVITLKSKLVSDFRKDF QFYKVREINNY | 981 |
| WP_034440723 | 915 | RD ROOFINROLVETROITKHVANLLSHHLNEK-----KEVG--EINIVLLKSALTSQFRKKE DFYKVREVNDY | 980 |
| AKQ21048 | 911 | LD KAGFIKRQLVETROITKHVAQILDSRMNTKYDENDKLIR--EVKVITLKSKLVSDFRKDF QFYKVREINNY | 981 |
| WP_004636532 | 916 | AD RAHFLNRQLVETROITKHVANLLDSQYNTAEEQ-----R---INIVLLKSSMTSRFRKEF KLYKVREINDY | 980 |
| WP_002364836 | 922 | ED KAHFIQROLVETROITKNVAGILDQRYNANSKE-----K--KVQIITLKASLTSQFRSIF GLYKVREVNDY | 987 |
| WP_016631044 | 873 | ED KAHFIQRQLVETROITKNVAGILDQRYNAKSKE-----K--KVQIITLKASLTSQFRSIF GLYKVREVNDY | 938 |
| EMS75795 | 655 | ED KARFIQROLVETRQITKHVAAILDQYFN-QPEE-SK-NK--GIRIITLKSSLVSQFRKTF GINKVREINNH | 722 |
| WP_002373311 | 922 | ED KAHFIQROLVETRQITKNVAGILDQRYNAKSKE-----K--KVQIITLKASLTSQFRSIF GLYKVREVNDY | 987 |
| WP_002378009 | 922 | ED KAHFIQROLVETROITKNVAGILDQRYNAKSKE-----K--KVQIITLKASLTSQFRSIF GLYKVREVNDY | 987 |
| WP_002407324 | 922 | ED KAHFIQROLVETRQITKNVAGILDQRYNAKSKE-----K--KVQIITLKASLTSQFRSIF GLYKVREVNDY | 987 |
| WP_002413717 | 922 | ED KAHFIQRQLVETROITKNVAGILNQRYNANSKE-----K--KVQIITLKASLTSQFRSIF GLYKVREVNDY | 987 |
| WP_010775580 | 924 | ED KAHFIQROLVETROITKNVAGILDORYNAKSKE-----K--KVQIITLKASLTSQFRSIF GLYKVREVNDY | 989 |
| WP_010818269 | 922 | ED KAHFIQROLVETROITKNVAGILDQRYNAKSKE-----K--KVQIITLKASLTSQFRSIF GLYKVREVNDY | 987 |
| WP_010824395 | 922 | ED KAHFIQROLVETROITKNVAGILDQLYNAKSKE-----K--KVQIITLKASLTSQFRSIF GLYKVREVNDY | 987 |
| WP_016622645 | 922 | ED KAHFIQROLVETROITKNVAGILDQRYNAKSKE-----K--KVQIITLKASLTSQFRSIF GLYKVREVNDY | 987 |
| WP_033624816 | 922 | ED KAHFIQROLVETROITKNVAGILDQRYNAKSKE-----K--KVQIITLKASLTSQFRSIF GLYKVREVNDY | 987 |
| WP_033625576 | 922 | ED KAHFIQROLVETROITKNVAGILDQRYNAKSKE-----K--KVQIITLKASLTSQFRSIF GLYKVREVNDY | 987 |
| WP_033789179 | 922 | ED KAHFIQROLVETROITKNVAGILDQRYNAKSKE-----K--KVQIITLKASLTSQFRSIF GLYKVREVNDY | 987 |
| WP_002310644 | 919 | ED KAGFIKRQLVETRQITKHVAGILHHRFN-KAEDTNEPIR--KVRIITLKSALVSQFRNRF GIYKVREINEY | 988 |
| WP_002312694 | 920 | ED KAGFIKRQLVETRQITKHVAGILHHREN-KAEDTNDPIR--KVRIITLKSALVSQFRNRF GIYKVREINEY | 989 |
| WP_002314015 | 920 | ED KAGFIKRQLVETROITKHVAGILHHRFN-KAEDTNEPIR--KVRIITLKSALVSQFRNRF GIYKVREINEY | 989 |
| WP_002320716 | 920 | ED KAGFIKRQLVETROITKHVAGILHHREN-KAEDTNEPIR--KVRIITLKSALVSQFRNRF GIYKVREINEY | 989 |
| WP_002330729 | 919 | ED KAGFIKRQLVETROITKHVAGILHHREN-KAEDTNEPIR--KVRIITLKSALVSQFRNRF GIYKVREINEY | 988 |
| WP_002335161 | 920 | ED KAGFIKRQLVETROITKHVAGILHHREN-KAEDTNEPIR--KVRIITLKSALVSQFRNRF GIYKVREINEY | 989 |
| WP_002345439 | 920 | ED KAGFIKRQLVETRQITKHVAGILHHREN-KAEDTNEPIR--KVRIITLKSALVSQFRNRF GIYKVREINEY | 989 |
| WP_034867970 | 911 | ED KARFIQRQLVETROITKHVANILHQSFN-QEEEGTD-CD--GVQIITLKATLTSQFRQTF GLYKVREINPH | 979 |
| WP_047937432 | 920 | ED KAGFIKRQLVETROITKHVAGILHHREN-KAEDTNEPIR--KVRIITLKSALVSQFRNRF GIYKVREINEY | 989 |
| WP_010720994 | 911 | ED KARFIQROLVETROITKHVANILHOSFN-QEEEGTD-CD--GVQIITLKATLTSQFRQTF GLYKVREINPH | 979 |
| WP_010737004 | 911 | ED KARFIQRQLVETROITKHVANILHQSFN-QEEEGTD-CD--GVQIITLKATLTSQFROTF GLYKVREINPH | 979 |
| WP_034700478 | 911 | ED KARFIQRQLVETROITKHVANILHQSFN-QEEEGTD-CD--GVQIITLKATLTSQFRQTF GLYKVREINPH | 979 |
| WP_007209003 | 912 | SD KAGFLKRQLVETRQITKHVATILDSKFNE--DSNNRDVQ-----IITLKSALVSEFRKTF NLYKVREINDL | 977 |
| WP_023519017 | 905 | AD KARFIQRQLVETRQITKHVANLLHQHFN-LPEEVSA-TE--KTSIITLKSTLTSQFROMF DIYKVREINHH | 973 |
| WP_010770040 | 915 | DD RAHFIKRQLVETROITKHVARILDORFNSQKDEEGKTIR--AVRVVTLKSSLTSQFRKQF AIHKVREINDY | 985 |
| WP_048604708 | 912 | DD KAGFIHRQLVETROITKNVARILHQRFNSEKDEEGNLIR--KVRIITLKSALTSQFRKNY GIYKIREINDY | 982 |
| WP_010750235 | 914 | DD KARFIQROLVETRQITKHVAAILHQYFN-QTQELEK-EK--DIRIITLKSSLVSQFRQVF GIHKVREINHH | 982 |
| AII16583 | 950 | LD KAGFIKRQLVETROITKHVAQILDSRMNTKYDENDKLIR--EVKVITLKSKLVSDFRKDF QFYKVREINNY | 1020 |
| WP_029073316 | 928 | KD KERFINRQIVETROITKHVAQIISNHYET-------------TKVVTVRADLSHAFRERY HIYKNRDINDF | 987 |
| WP_031589969 | 928 | KD QERFINROIVETRQITKHVAQIIDNHYEN-------------TKVVTVRADLSHQFRERY HIYKNRDINDF | 987 |
| KDA45870 | 903 | KL KERFIERQLVETRQITKYVAQLLDORLN--YDGNGVELD-eKIAIVILKAQLASQFRSEF KLRKVRALNNL | 972 |
| WP_039099354 | 924 | -D MKGFINROLVETROVIKLATNLLMEQYGED-----------NIELITVKSGLTHQMRTEF DFPKNRNLNNH | 990 |
| AKP02966 | 926 | KD KLGFIHRQLVQTSQMVKGVANILNSMYK---NQGTTCIQ--------ARANLSTAFRKAL ELVKNRNINDF | 999 |
| WP_010991369 | 914 | AD KARFIHRQLVETRQITKNVANILHQRFNYEKDDHGNTMK--QVRIVTLKSALVSQFRKOF QLYKVRDVNDY | 984 |
| WP_033838504 | 914 | AD KARFIHRQLVETROITKNVANILHQRFNYEKDDHGNTMK--QVRIVTLKSALVSQFRKOF QLYKVRDVNDY | 984 |
| EHN60060 | 917 | AD KARFIHRQLVETROITKNVANILHQRFNYEKDDHGNTMK--QVRIVTLKSALVSQFRKOF QLYKVRDVNDY | 987 |
| EFR89594 | 683 | AD KARFIHRQLVETRQITKNVANILHQRFNYGKDDHGNTMK--QVRIVTLKSALVSQFRKOF QLYKVRGVNDY | 753 |
| WP_038409211 | 914 | AD KANFIQRQLVETROITKNVANILYQRFNCKQDENGNEVE--QVRIVTLKSTLVSQFRKOF QLYKVREVNDY | 984 |
| EFR95520 | 533 | AD KANFIQRQLVETROITKNVANILYQRFNCKQDENGNEVE--QVRIVTLKSTLVSQFRKOF QLYKVREVNDY | 603 |
| WP_003723650 | 914 | AD KARFIHRQLVETRQITKNVANILYQRFNKETDNHGNTME--QVRIVTLKSALVSQFRKOF QLYKVREVNGY | 984 |
| WP_003727705 | 914 | AD KARFIHRQLVETRQITKNVANILHQRFNKETDNHGNTME--QVRIVTLKSALVSQFRKOF QLYKVREVNDY | 984 |
| WP_003730785 | 914 | AD KARFIHRQLVETRQITKNVANILHQRFNKETDNHGNTME--QVRIVTLKSALVSQFRKOF QLYKVREVNDY | 984 |
| WP_003733029 | 914 | AD KARFIHRQLVETRQITKNVANILHQRFNYKTDGNKDTME--TVRIVTLKSALVSQFRKOF QFYKVREVNDY | 984 |
| WP_003739838 | 914 | AD KATFIHRQLVETROITKNVANILHQRFNNETDNHGNNME--QVRIVMLKSALVSQFRKOF QLYKVREVNDY | 984 |
| WP_014601172 | 914 | AD KARFIHRQLVETRQITKNVANILHQRENNETDNHGNTME--QVRIVTLKSALVSOFRKOF QLYKVREVNDY | 984 |
| WP_023548323 | 914 | AD KARFIHRQLVETRQITKNVANILHQRFNYKTDDNEDTME--PVRIVTLKSALVSQFRKOF QLYKVREVNDY | 984 |
| WP_031665337 | 914 | AD KARFIHRQLVETRQITKNVANILHQRFNKETDNHGNTME--QVRIVTLKSALVSQFRKOF QLYKVREVNDY | 984 |
| WP_031669209 | 914 | AD KARFIHRQLVETROITKNVANILHQRFNYKTDGNKDTME--TVRIVTLKSALVSQFRKOF QFYKVREVNDY | 984 |
| WP_033920898 | 914 | AD KARFIHRQLVETRQITKNVANILHQRFNYKTDDNEDTME--PVRIVTLKSALVSQFRKOF QLYKVREVNDY | 984 |
| AKI42028 | 917 | AD KARFIHRQLVETROITKNVANILHQRFNNETDNHGNTME--QVRIVTLKSALVSQFRKOF QLYKVREVNDY | 987 |
| AKI50529 | 917 | AD KARFIHRQLVETROITKNVANILHQRFNYKTDDNEDTME--PVRIVTLKSALVSQFRKOF QLYKVREVNDY | 987 |
| EFR83390 | 362 | AD KARFIHRQLVETRQITKNVANILHQRENNETDNHGNTME--QVRIVTLKSALVSQFRKOF QLYKVREVNDY | 432 |
| WP_046323366 | 914 | AD KARFIHRQLVETRQITKNVANILHQRFNCKKDESGNVIE--QVRIVTLKAALVSQFRKOF QLYKVREVNDY | 984 |
| AKE81011 | 927 | LD KAGFIKRQLVETROITKHVAQILDSRMNTKYDENDKLIR--EVKVITLKSKLVSDERKDF QFYKVREINNY | 997 |
| CUO82355 | 921 | RD EERFINROLVETRQITKNVTQIIEDHYST-------------TKVAAIRANLSHEFRVKN HIYKNRDINDY | 980 |
| WP_033162887 | 923 | KD KERFINROLVETRQIIKNVAVIINDHYTN-------------TNIVTVRAELSHQFRERY KIYKNRDINDF | 982 |
| AGZ01981 | 944 | LD KAGFIKRQLVETROITKHVAQILDSRMNTKYDENDKLIR--EVKVITLKSKLVSDFRKDF QFYKVREINNY | 1014 |
| AKA60242 | 911 | LD KAGFIKRQLVETROITKHVAQILDSRMNTKYDENDKLIR--EVKVITLKSKLVSDERKDE QFYKVREINNY | 981 |
| AKS40380 | 911 | LD KAGFIKRQLVETROITKHVAQILDSRMNTKYDENDKLIR--EVKVITLKSKLVSDERKDF QFYKVREINNY | 981 |
| 4UN5_B | 915 | LD KAGFIKRQLVETROITKHVAQILDSRMNTKYDENDKLIR--EVKVITLKSKLVSDFRKDF QFYKVREINNY | 985 |
| WP_010922251 | 982 | 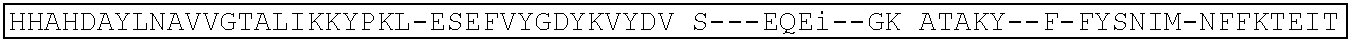 | 1051 |
| WP_039695303 | 992 | HHAHDAYLNAVVGTALLKKYPKL-ASEFVYGEYKKYDI S---SD------ KATAK--YfFYSNLM-NFFKTKVK | 1058 |
| WP_045635197 | 984 | HHAHDAYLNAVVAKAILKKYPKL-EPEFVYGEYQKYDL SkdpKEV---EK ATEKY--F-FYSNLL-NFFKEEVH | 1055 |
| 5AXW_A | 703 | HHAEDALI--------------IaNADFIFKEWKKLDK Nq-mFE----EK ETEQEykEiFITPHQiKHIKDFKD | 771 |
| WP_009880683 | 666 | HHAHDAYLNAVVGTALIKKYPKL-ESEFVYGDYKVYDI S---EQEi--GK ATAKY--F-FYSNIM-NFFKTEIT | 735 |
| WP_010922251 | 982 | HHAHDAYLNAVVGTALIKKYPKL-ESEFVYGDYKVYDV S---EQEi--GK ATAKY--F-FYSNIM-NFFKTEIT | 1051 |
| WP_011054416 | 982 | HHAHDAYLNAVVGTALIKKYPKL-ESEFVYGDYKVYDV S---EQEi--GK ATAKY--F-FYSNIM-NFFKTEIT | 1051 |
| WP_011284745 | 982 | HHAHDAYLNAVVGTALIKKYPKL-ESEFVYGDYKVYDV S---EQEi--GK ATAKY--F-FYSNIM-NFFKTEIT | 1051 |
| WP_011285506 | 982 | HHAHDAYLNAVVGTALIKKYPKL-ESEFVYGDYKVYDV S---EQEi--GK ATAKY--F-FYSNIM-NFFKTEIT | 1051 |
| WP_011527619 | 982 | HHAHDAYLNAVVGTALIKKYPKL-ESEFVYGDYKVYDV S---EQEi--GK ATAKY--F-FYSNIM-NFFKTEIT | 1051 |
| WP_012560673 | 982 | HHAHDAYLNAVVGTALIKKYPKL-ESEFVYGDYKVYDI S---EQEi--GK ATAKY--F-FYSNIM-NFFKTEIT | 1051 |
| WP_014407541 | 981 | HHAHDAYLNAVVGTALIKKYPKL-ESEFVYGDYKVYDV S---EQEi--GK ATAKY--F-FYSNIM-NFFKTEIT | 1050 |
| WP_020905136 | 982 | HHAHDAYLNAVVGTALIKKYPKL-ESEFVYGDYKVYDV S---EQEi--GK ATAKY--F-FYSNIM-NFFKTEIT | 1051 |
| WP_023080005 | 981 | HHAHDAYLNAVVGTALIKKYPKL-ESEFVYGDYKVYDV S---EQEi--GK ATAKY--F-FYSNIM-NFFKTEIT | 1050 |
| WP_023610282 | 981 | HHAHDAYLNAVVGTALIKKYPKL-ESEFVYGDYKVYDV S---EQEi--GK ATAKY--F-FYSNIM-NFFKTEIT | 1050 |
| WP_030125963 | 982 | HHAHDAYLNAVVGTALIKKYPKL-ESEFVYGDYKVYDV S---EQEi--GK ATAKY--F-FYSNIM-NFFKTEIT | 1051 |
| WP_030126706 | 982 | HHAHDAYLNAVVGTALIKKYPKL-ESEFVYGDYKVYDV S---EQEi--GK ATAKY--F-FYSNIM-NFFKTEIT | 1051 |
| WP_031488318 | 982 | HHAHDAYLNAVVGTALIKKYPKL-ESEFVYGDYKVYDV S---EQEi--GK ATAKY--F-FYSNIM-NFFKTEIT | 1051 |
| WP_032460140 | 982 | HHAHDAYLNAVVGTALIKKYPKL-ESEFVYGDYKVYDI S---EQEi--GK ATAKY--F-FYSNIM-NFFKTEIT | 1051 |
| WP_032461047 | 982 | HHAHDAYLNAVVGTALIKKYPKL-ESEFVYGDYKVYDI S---EQEi--GK ATAKY--F-FYSNIM-NFFKTEIT | 1051 |
| WP_032462016 | 982 | HHAHDAYLNAVVGTALIKKYPKL-ESEFVYGDYKVYDV S---EQEi--GK ATAKY--F-FYSNIM-NFFKTEIT | 1051 |
| WP_032462936 | 982 | HHAHDAYLNAVVGTALIKKYPKL-ESEFVYGDYKVYDV S---EQEi--GK ATAKY--F-FYSNIM-NFFKTEIT | 1051 |
| WP_032464890 | 982 | HHAHDAYLNAVVGTALIKKYPKL-ESEFVYGDYKVYDV S---EQEi--GK ATAKY--F-FYSNIM-NFFKTEIT | 1051 |
| WP_033888930 | 807 | HHAHDAYLNAVVGTALIKKYPKL-ESEFVYGDYKVYDV S---EQEi--GK ATAKY--F-FYSNIM-NFFKTEIT | 876 |
| WP_038431314 | 982 | HHAHDAYLNAVVGTALIKKYPKL-ESEFVYGDYKVYDV S---EQEi--GK ATAKY--F-FYSNIM-NFFKTEIT | 1051 |
| WP_038432938 | 981 | HHAHDAYLNAVVGTALIKKYPKL-ESEFVYGDYKVYDV S---EQEi--GK ATAKY--F-FYSNIM-NFFKTEIT | 1050 |
| WP_038434062 | 982 | HHAHDAYLNAVVGTALIKKYPKL-ESEFVYGDYKVYDV S---EQEi--GK ATAKY--F-FYSNIM-NFFKTEIT | 1051 |
| BAQ51233 | 893 | HHAHDAYLNAVVGTALIKKYPKL-ESEFVYGDYKVYDV S---EQEi--GK ATAKY--F-FYSNIM-NFFKTEIT | 962 |
| KGE60162 | 157 | HHAHDAYLNAVVGTALIKKYPKL-ESEFVYGDYKVYDV S---EQEi--GK ATAKY--F-FYSNIM-NFFKTEIT | 226 |
| KGE60856 | | -------------------------------------- ------------ ------------------------ | |
| WP_002989955 | 982 | HHAHDAYLNAVVGTALIKKYPKL-ESEFVYGDYKVYDV S---EQEi--GK ATAKY--F-FYSNIM-NFFKTEIT | 1051 |
| WP_003030002 | 981 | HHAHDAYLNAVVGNALLLKYPQL-EPEFVYGEYPKYN- S---YR---sRK SATEK--FIFYSNIL-RFFKKE-- | 1041 |
| WP_003065552 | 992 | HHAHDAYLNAVVGTALLKKYPKL-ASEFVYGEYKKYDI S---SD------ KATAK--YfFYSNLM-NFFKRVIR | 1058 |
| WP_001040076 | 987 | HHAHDAYLNAVVAKAILTKYPOL-EPEFVYGDYPKYN- S---YKT---RK ATEKL--F-FYSNIM-NFFKTKVT | 1049 |
| WP_001040078 | 987 | HHAHDAYLNAVVAKAILTKYPQL-EPEFVYGLYRRKK- L---SKI---VR ATRKM--F-FYSNLM-NMFKRVVR | 1057 |
| WP_001040080 | 987 | HHAHDAYLNAVVAKAILTKYPOL-EPEFVYGDYPKYN- S---YKT---RK ATEKL--F-FYSNIM-NFFKTKVT | 1049 |
| WP_001040081 | 987 | HHAHDAYLNAVVAKAILTKYPOL-EPEFVYGDYPKYN- S---YKT---RK ATEKL--F-FYSNIM-NFFKTKVT | 1049 |
| WP_001040083 | 987 | HHAHDAYLNAVVAKAILTKYPOL-EPEFVYGDYPKYN- S---YKT---RK ATEKL--F-FYSNIM-NFFKTKVT | 1049 |
| WP_001040085 | 987 | HHAHDAYLNAVVAKAILTKYPQL-EPEFVYGDYPKYN- S---YKT---RK ATEKL--F-FYSNIM-NFFKTKVT | 1049 |
| WP_001040087 | 987 | HHAHDAYLNAVVAKAILTKYPOL-EPEFVYGDYPKYN- S---YKT---RK ATEKL--F-FYSNIM-NFFKTKVT | 1049 |
| WP_001040088 | 987 | HHAHDAYLNAVVAKAILTKYPOL-EPEFVYGDYPKYN- S---YKT---RK ATEKL--F-FYSNIM-NFFKTKVT | 1049 |
| WP_001040089 | 987 | HHAHDAYLNAVVAKAILTKYPOL-EPEFVYGDYPKYN- S---YKT---RK ATEKL--F-FYSNIM-NFFKTKVT | 1049 |
| WP_001040090 | 987 | HHAHDAYLNAVVAKAILTKYPOL-EPEFVYGDYPKYN- S---YKT---RK ATEKL--F-FYSNIM-NFFKTKVT | 1049 |
| WP_001040091 | 987 | HHAHDAYLNAVVAKAILTKYPOL-EPEFVYGDYPKYN- S---YKT---RK ATEKL--F-FYSNIM-NFFKTKVT | 1049 |
| WP_001040092 | 987 | HHAHDAYLNAVVAKAILTKYPOL-EPEFVYGDYPKYN- S---YKT---RK ATEKL--F-FYSNIM-NFFKTKVT | 1049 |
| WP_001040094 | 987 | HHAHDAYLNAVVAKAILTKYPOL-EPEFVYGDYPKYN- S---YKT---RK ATEKL--F-FYSNIM-NFFKTKVT | 1049 |
| WP_001040095 | 987 | HHAHDAYLNAVVAKAILTKYPOL-EPEFVYGDYPKYN- S---YKT---RK ATEKL--F-FYSNIM-NFFKTKVT | 1049 |
| WP_001040096 | 987 | HHAHDAYLNAVVAKAILTKYPOL-EPEFVYGDYPKYN- S---YKT---RK ATEKL--F-FYSNIM-NFFKTKVT | 1049 |
| WP_001040097 | 987 | HHAHDAYLNAVVAKAILTKYPOL-EPEFVYGDYPKYN- S---YKT---RK ATEKL--F-FYSNIM-NFFKTKVT | 1049 |
| WP_001040098 | 987 | HHAHDAYLNAVVAKAILTKYPOL-EPEFVYGDYPKYN- S---YKT---RK ATEKL--F-FYSNIM-NFFKTKVT | 1049 |
| WP_001040099 | 987 | HHAHDAYLNAVVAKAILTKYPOL-EPEFVYGDYPKYN- S---YKT---RK ATEKL--F-FYSNIM-NFFKTKVT | 1049 |
| WP_001040100 | 987 | HHAHDAYLNAVVAKAILTKYPOL-EPEFVYGDYPKYN- S---YKT---RK ATEKL--F-FYSNIM-NFFKTKVT | 1049 |
| WP_001040104 | 987 | HHAHDAYLNAVVAKAILTKYPOL-EPEFVYGDYPKYN- S---YKT---RK ATEKL--F-FYSNIM-NFFKTKVT | 1049 |
| WP_001040105 | 987 | HHAHDAYLNAVVAKAILTKYPOL-EPEFVYGDYPKYN- S---YKT---RK ATEKL--F-FYSNIM-NFFKTKVT | 1049 |
| WP_001040106 | 987 | HHAHDAYLNAVVAKAILTKYPOL-EPEFVYGDYPKYN- S---YKT---RK ATEKL--F-FYSNIM-NFFKTKVT | 1049 |
| WP_001040107 | 987 | HHAHDAYLNAVVAKAILTKYPOL-EPEFVYGDYPKYN- S---YKT---RK ATEKL--F-FYSNIM-NFFKTKVT | 1049 |
| WP_001040108 | 987 | HHAHDAYLNAVVAKAILTKYPOL-EPEFVYGDYPKYN- S---YKT---RK ATEKL--F-FYSNIM-NFFKTKVT | 1049 |
| WP_001040109 | 987 | HHAHDAYLNAVVAKAILTKYPOL-EPEFVYGDYPKYN- S---YKT---RK ATEKL--F-FYSNIM-NFFKTKVT | 1049 |
| WP_001040110 | 987 | HHAHDAYLNAVVAKAILTKYPOL-EPEFVYGDYPKYN- S---YKT---RK ATEKL--F-FYSNIM-NFFKTKVT | 1049 |
| WP_015058523 | 987 | HHAHDAYLNAVVAKAILTKYPOL-EPEFVYGDYPKYN- S---YKT---RK ATEKL--F-FYSNIM-NFFKTKVT | 1049 |
| WP_017643650 | 987 | HHAHDAYLNAVVAKAILTKYPOL-EPEFVYGDYPKYN- S---YKT---RK ATEKL--F-FYSNIM-NFFKTKVT | 1049 |
| WP_017647151 | 987 | HHAHDAYLNAVVAKAILTKYPOL-EPEFVYGDYPKYN- S---YKT---RK ATEKL--F-FYSNIM-NFFKTKVT | 1049 |
| WP_017648376 | 987 | HHAHDAYLNAVVAKAILTKYPOL-EPEFVYGDYPKYN- S---YKT---RK ATEKL--F-FYSNIM-NFFKTKVT | 1049 |
| WP_017649527 | 987 | HHAHDAYLNAVVAKAILTKYPOL-EPEFVYGDYPKYN- S---YKT---RK ATEKL--F-FYSNIM-NFFKTKVT | 1049 |
| WP_017771611 | 987 | HHAHDAYLNAVVAKAILTKYPOL-EPEFVYGDYPKYN- S---YKT---RK ATEKL--F-FYSNIM-NFFKTKVT | 1049 |
| WP_017771984 | 987 | HHAHDAYLNAVVAKAILTKYPOL-EPEFVYGDYPKYN- S---YKT---RK ATEKL--F-FYSNIM-NFFKTKVT | 1049 |
| CFQ25032 | 987 | HHAHDAYLNAVVAKAILTKYPOL-EPEFVYGDYPKYN- S---YKT---RK ATEKL--F-FYSNIM-NFFKTKVT | 1049 |
| CVF16040 | 987 | HHAHDAYLNAVVAKAILTKYPOL-EPEFVYGDYPKYN- S---YKT---RK ATEKL--F-FYSNIM-NFFKTKVT | 1049 |
| KLJ37842 | 987 | HHAHDAYLNAVVAKAILTKYPOL-EPEFVYGDYPKYN- S---YKT---RK ATEKL--F-FYSNIM-NFFKTKVT | 1049 |
| KLJ72361 | 987 | HHAHDAYLNAVVAKAILTKYPOL-EPEFVYGDYPKYN- S---YKT---RK ATEKL--F-FYSNIM-NFFKTKVT | 1049 |
| KLL20707 | 1001 | HHAHDAYLNAVVAKAILTKYPOL-EPEFVYGDYPKYN- S---YKT---RK ATEKL--F-FYSNIM-NFFKTKVT | 1063 |
| KLL42645 | 987 | HHAHDAYLNAVVAKAILTKYPOL-EPEFVYGDYPKYN- S---YKT---RK ATEKL--F-FYSNIM-NFFKTKVT | 1049 |
| WP_047207273 | 987 | HHAHDAYLNAVVAKAILTKYPOL-EPEFVYGDYPKYN- S---YKT---RK ATEKL--F-FYSNIM-NFFKTKVT | 1049 |
| WP_047209694 | 987 | HHAHDAYLNAVVAKAILTKYPOL-EPEFVYGDYPKYN- S---YKT---RK ATEKL--F-FYSNIM-NFFKTKVT | 1049 |
| WP_050198062 | 987 | HHAHDAYLNAVVAKAILTKYPOL-EPEFVYGDYPKYN- S---YKT---RK ATEKL--F-FYSNIM-NFFKTKVT | 1049 |
| WP_050201642 | 987 | HHAHDAYLNAVVAKAILTKYPOL-EPEFVYGDYPKYN- S---YKT---RK ATEKL--F-FYSNIM-NFFKTKVT | 1049 |
| WP_050204027 | 987 | HHAHDAYLNAVVAKAILTKYPOL-EPEFVYGDYPKYN- S---YKT---RK ATEKL--F-FYSNIM-NFFKTKVT | 1049 |
| WP_050881965 | 987 | HHAHDAYLNAVVAKAILTKYPOL-EPEFVYGDYPKYN- S---YKT---RK ATEKL--F-FYSNIM-NFFKTKVT | 1049 |
| WP_050886065 | 987 | HHAHDAYLNAVVAKAILTKYPOL-EPEFVYGDYPKYN- S---YKT---RK ATEKL--F-FYSNIM-NFFKTKVT | 1049 |
| AHN30376 | 987 | HHAHDAYLNAVVAKAILTKYPQL-EPEFVYGDYPKYN- S---YKT---RK ATEKL--F-FYSNIM-NFFKTKVT | 1049 |
| EAO78426 | 987 | HHAHDAYLNAVVAKAILTKYPOL-EPEFVYGDYPKYN- S---YKT---RK ATEKL--F-FYSNIM-NFFKTKVT | 1049 |
| CCW42055 | 987 | HHAHDAYLNAVVAKAILTKYPOL-EPEFVYGDYPKYN- S---YKT---RK ATEKL--F-FYSNIM-NFFKTKVT | 1049 |
| WP_003041502 | 981 | HHAHDAYLNAVIGTALLKKYPKL-ASEFVYGEFKKYDV S---DK---eIG KATAK--YfFYSNLM-NFFKKEVK | 1050 |
| WP_037593752 | 982 | HHAHDAYLNAVVGNALLLKYPOL-EPEFVYGEYPKYN- S---YR---SRK SATEK--FlFYSNIL-RFFKKE-- | 1042 |
| WP_049516684 | 982 | HHAHDAYLNAVVGNALLLKYPOL-EPEFVYGEYPKYN- S---YR---SRK SATEK--FlFYSNIL-RFFKKE-- | 1042 |
| GAD46167 | 981 | HHAHDAYLNAVVGNALLLKYPOL-EPEFVYGEYPKYN- S---YR---SRK SATEK--FlFYSNIL-RFFKKE-- | 1041 |
| WP_018363470 | 990 | HHAHDAYLNAVVGTALLKKYPKL-APEFVYGEYKKYDV S---SDDhseMG KATAK--YfFYSNLM-NFFKRNIR | 1062 |
| WP_003043819 | 991 | HHAHDAYLNAVVGTALIKKYPKL-ESEFVYGDYKVYDV S---EQEi--GK ATAKR--F-FYSNIM-NFFKTEVK | 1060 |
| WP_006269658 | 981 | HHAHDAYLNAVVGNALLLKYPOL-EPEFVYGEYPKYN- S---YR---SRK SATEK--FlFYSNIL-RFFKKE-- | 1041 |
| WP_048800889 | 981 | HHAHDAYLNAVVGTALLKKYPKL-TSEFVYGEYKKYDV S---DND--eIG KATAK--YfFYSNLM-NFFKKEVK | 1051 |
| WP_012767106 | 981 | HHAHDAYLNAVVGTALIKKYTKL-ESEFVYGDYKVYDV S---EQEi--GK ATAKR--F-FYSNIM-NFFKTEIT | 1050 |
| WP_014612333 | 981 | HHAHDAYLNAVVGTALIKKYPKL-ESEFVYGDYKVYDV S---EQEi--GK ATAKR--F-FYSNIM-NFFKTEIT | 1050 |
| WP_015017095 | 981 | HHAHDAYLNAVVGTALIKKYPKL-ESEFVYGDYKVYDV S---EQEi--GK ATAKR--F-FYSNIM-NFFKTEIT | 1050 |
| WP_015057649 | 981 | HHAHDAYLNAVVGTALIKKYPKL-ESEFVYGDYKVYDV S---EQEi--GK ATAKR--F-FYSNIM-NFFKTEIT | 1050 |
| WP_048327215 | 981 | HHAHDAYLNAVVGTALIKKYPKL-ESEFVYGDYKVYDV S---EQEi--GK ATAKR--F-FYSNIM-NFFKTEIT | 1050 |
| WP_049519324 | 981 | HHAHDAYLNAVVGTALIKKYPKL-ESEFVYGDYKVYDV S---EQEi--GK ATAKR--F-FYSNIM-NFFKTEIT | 1050 |
| WP_012515931 | 981 | HHAHDAYLNAVVAKAILGKYPQL-APEFVYGDYPKYN- S---FKEr--QK ATQKM--L-FYSNIL-KFFKDQES | 1043 |
| WP_021320964 | 981 | HHAHDAYLNAVVAKAILGKYPQL-APEFVYGDYPKYN- S---FKEr--QK ATQKT--L-FYSNIL-KFFKDQES | 1043 |
| WP_037581760 | 981 | HHAHDAYLNAVVAKAILGKYPQL-APEFVYGDYPKYN- S---FKEr--QK ATQKT--L-FYSNIL-KKFKDQES | 1043 |
| WP_004232481 | 989 | HHAHDAYLNAVVGTALLKKYPKL-APEFVYGEYKKYDV S---SDNhseLG KATAK--YfFYSNLM-NFFKTEVK | 1061 |
| WP_009854540 | 990 | HHAHDAYLNAVVGTALLKKYPKL-ASEFVYGEYKKYDI S---SD------ KATAK--YfFYSNLM-NFFKTKVK | 1056 |
| WP_012962174 | 990 | HHAHDAYLNAVVGTALLKKYPKL-APEFVYGEYKKYDI S---GD------ KATAK--YfFYSNLM-NFFKRVIR | 1056 |
| WP_039695303 | 992 | HHAHDAYLNAVVGTALLKKYPKL-ASEFVYGEYKKYDI S---SD------ KATAK--YfFYSNLM-NFFKTKVK | 1058 |
| WP_014334983 | 989 | HHAHDAYLNAVVGTALLKKYPKL-TPEFVYGEYKKYDV S---SDDyseMG KATAK--YfFYSNLM-NFFKTEVK | 1061 |
| WP_003099269 | 982 | HHAQDAYLNAVVGTALLKKYPKL-EAEFVYGDYKHYDL P---DSS1--GK ATTRM--F-FYSNLM-NFFKKEIK | 1051 |
| AHY15608 | 982 | HHAQDAYLNAVVGTALLKKYPKL-EAEFVYGDYKHYDL P---DSS1--GK ATTRM--F-FYSNLM-NFFKKEIK | 1051 |
| AHY17476 | 982 | HHAQDAYLNAVVGTALLKKYPKL-EAEFVYGDYKHYDL P---DSS1--GK ATTRM--F-FYSNLM-NFFKKEIK | 1051 |
| ESR09100 | | -------------------------------------- ------------ ------------------------ | |
| AGM98575 | 982 | HHAQDAYLNAVVGTALLKKYPKL-EAEFVYGDYKHYDL P---DSS1--GK ATTRM--F-FYSNLM-NFFKKEIK | 1051 |
| ALF27331 | 982 | HHAHDAYLNAVIGKALLGVYPQL-EPEFVYGDYPHFH- G---HK---eNK ATAKK--F-FYSNIM-NFFKKD-- | 1041 |
| WP_018372492 | 995 | HHAHDAYLNAVVAQALLKVYPKF-ERELVYGSYVKESI ----FS----RK ATERM---rMYNNIL-KFISKD-- | 1055 |
| WP_045618028 | 985 | HHAHDPYLNAVVAKAILKKYPKL-EPEFVYGDYQKYDL TkdpKEV---EK ATEKY--F-FYSNLL-NFFKEEVH | 1056 |
| WP_045635197 | 984 | HHAHDAYLNAVVAKAILKKYPKL-EPEFVYGEYQKYDL SkdpKEV---EK ATEKY--F-FYSNLL-NFFKEEVH | 1055 |
| WP_002263549 | 982 | HHAHDAYLNAVIGKALLGVYPQL-EPEFVYGDYPHFH- G---HK---eNK ATAKK--F-FYSNIM-NFFKKD-- | 1041 |
| WP_002263887 | 982 | HHAHDAYLNAVIGKALLGVYPQL-EPEFVYGDYPHFH- G---HK---eNK ATAKK--F-FYSNIM-NFFKKD-- | 1041 |
| WP_002264920 | 982 | HHAHDAYLNAVIGKALLGVYPQL-EPEFVYGDYPHFH- G---HK---eNK ATAKK--F-FYSNIM-NFFKKD-- | 1041 |
| WP_002269043 | 982 | HHAHDAYLNAVIGKALLGVYPQL-EPEFVYGDYPHFH- G---HK---eNK ATAKK--F-FYSNIM-NFFKKD-- | 1041 |
| WP_002269448 | 982 | HHAHDAYLNAVIGKALLGVYPQL-EPEFVYGDYPHFH- G---HK---eNK ATAKK--F-FYSNIM-NFFKKD-- | 1041 |
| WP_002271977 | 982 | HHAHDAYLNAVIGKALLGVYPQL-EPEFVYGDYPHFH- G---HK---eNK ATAKK--F-FYSNIM-NFFKKD-- | 1041 |
| WP_002272766 | 982 | HHAHDAYLNAVIGKALLGVYPQL-EPEFVYGDYPHFH- G---HE---eNK ATAKK--F-FYSNIM-NFFKKD-- | 1041 |
| WP_002273241 | 982 | HHAHDAYLNAVIGKALLGVYPQL-EPEFVYGDYPHFH- G---HK---eNK ATAKK--F-FYSNIM-NFFKKD-- | 1041 |
| WP_002275430 | 982 | HHAHDAYLNAVIGKALLGVYPQL-EPEFVYGDYPHFH- G---HE---eNK ATAKK--F-FYSNIM-NFFKKD-- | 1041 |
| WP_002275430 | 982 | HHAHDAYLNAVIGKALLGVYPQL-EPEFVYGDYPHFH- G---HK---eNK ATAKK--F-FYSNIM-NFFKKD-- | 1041 |
| WP_002276448 | 982 | HHAHDAYLNAVIGKALLGVYPQL-EPEFVYGDYPHFH- G---YR---eRK ATAKK--F-FYSNIM-NFFKKD-- | 1041 |
| WP_002277050 | 984 | HHAHDAYLNAVVVKALLVKYPKL-EPEFVYGEYPLYN- S---YR---eRK ATQKM--F-FYSNIM-NMFKSKVK | 1046 |
| WP_002277364 | 982 | HHAHDAYLNAVIGKALLGVYPQL-EPEFVYGDYPHFH- G---HK---eNK ATAKK--F-FYSNIM-NFFKKD-- | 1041 |
| WP_002279025 | 982 | HHAHDAYLNAVIGKALLGVYPQL-EPEFVYGDYPHFH- G---HE---eNK ATAKK--F-FYSNIM-NFFKKD-- | 1041 |
| WP_002279859 | 982 | HHAHDAYLNAVIGKALLGVYPQL-EPEFVYGDYPHFH- G---HK---eNK ATAKK--F-FYSNIM-NFFKKD-- | 1041 |
| WP_002280230 | 982 | HHAHDAYLNAVIGKALLGVYPQL-EPEFVYGDYPHFH- G---HK---eNK ATAKK--F-FYSNIM-NFFKKD-- | 1041 |
| WP_002281696 | 982 | HHAHDAYLNAVIGKALLGVYPQL-EPEFVYGDYPHFH- G---HK---eNK ATAKK--F-FYSNIM-NFFKKD-- | 1041 |
| WP_002282247 | 984 | HHAHDAYLNAVVVKALLVKYPKL-EPEFVYGDYPHFH- S---YR---eRK ATQKM--F-FYSNIM-NMFKSKVK | 1046 |
| WP_002282906 | 982 | HHAHDAYLNAVIGKALLGVYPQL-EPEFVYGDYPHFH- G---HK---eNK ATAKK--F-FYSNIM-NFFKKD-- | 1041 |
| WP_002283846 | 982 | HHAHDAYLNAVIGKALLGVYPQL-EPEFVYGDYPHFH- G---HK---eNK ATAKK--F-FYSNIM-NFFKKD-- | 1041 |
| WP_002287255 | 982 | HHAHDAYLNAVIGKALLGVYPQL-EPEFVYGDYPHFH- G---HK---eNK ATAKK--F-FYSNIM-NFFKKD-- | 1041 |
| WP_002288990 | 982 | HHAHDAYLNAVIGKALLGVYPQL-EPEFVYGDYPHFH- G---HK---eNK ATAKK--F-FYSNIM-NFFKKD-- | 1041 |
| WP_002289641 | 982 | HHAHDAYLNAVIGKALLGVYPQL-EPEFVYGDYPHFH- G---HK---eNK ATAKK--F-FYSNIM-NFFKKD-- | 1041 |
| WP_002290427 | 982 | HHAHDAYLNAVIGKALLGVYPQL-EPEFVYGDYPHFH- G---HK---eNK ATAKK--F-FYSNIM-NFFKKD-- | 1041 |
| WP_002295753 | 982 | HHAHDAYLNAVIGKALLGVYPQL-EPEFVYGDYPHFH- G---HK---eNK ATAKK--F-FYSNIM-NFFKKD-- | 1041 |
| WP_002296423 | 982 | HHAHDAYLNAVIGKALLGVYPQL-EPEFVYGDYPHFH- G---HK---eNK ATAKK--F-FYSNIM-NFFKKD-- | 1041 |
| WP_002304487 | 996 | HHAHDAYLNAVIGKALLGVYPQL-EPEFVYGDYPHFH- G---HK---eNK ATAKK--F-FYSNIM-NFFKKG-- | 1055 |
| WP_002305844 | 982 | HHAHDAYLNAVIGKALLGVYPQL-EPEFVYGDYPHFH- G---HK---eNK ATAKK--F-FYSNIM-NFFKKD-- | 1041 |
| WP_002307203 | 982 | HHAHDAYLNAVIGKALLGVYPQL-EPEFVYGDYPHFH- G---HK---eNK ATAKK--F-FYSNIM-NFFKKD-- | 1041 |
| WP_002310390 | 982 | HHAHDAYLNAVIGKALLGVYPQL-EPEFVYGDYPHFH- G---HK---eNK ATAKK--F-FYSNIM-NFFKKD-- | 1041 |
| WP_002310390 | 982 | HHAHDAYLNAVIGKALLGVYPQL-EPEFVYGDYPHFH- G---HK---eNK ATAKK--F-FYSNIM-NFFKKD-- | 1041 |
| WP_002352408 | 982 | HHAHDAYLNAVIGKALLGVYPQL-EPEFVYGDYPHFH- G---HK---eNK ATAKK--F-FYSNIM-NFFKKD-- | 1041 |
| WP_012997688 | 982 | HHAHDAYLNAVIGKALLGVYPQL-EPEFVYGDYPHFH- G---HK---eNK ATAKK--F-FYSNIM-NFFKKD-- | 1041 |
| WP_014677909 | 982 | HHAHDAYLNAVIGKALLGVYPQL-EPEFVYGDYPHFH- G---HK---eNK ATAKK--F-FYSNIM-NFFKKD-- | 1041 |
| WP_019312892 | 982 | HHAHDAYLNAVIGKALLGVYPQL-EPEFVYGDYPHFH- G---HK---eNK ATAKK--F-FYSNIM-NFFKKD-- | 1041 |
| WP_019313659 | 982 | HHAHDAYLNAVIGKALLGVYPQL-EPEFVYGDYPHFH- G---HK---eNK ATAKK--F-FYSNIM-NFFKKD-- | 1041 |
| WP_019314093 | 982 | HHAHDAYLNAVIGKALLGVYPQL-EPEFVYGDYPHFH- G---HE---eNK ATAKK--F-FYSNIM-NFFKKD-- | 1041 |
| WP_019315370 | 982 | HHAHDAYLNAVIGKALLGVYPQL-EPEFVYGDYPHFH- G---HK---eNK ATAKK--F-FYSNIM-NFFKKD-- | 1041 |
| WP_019803776 | 982 | HHAHDAYLNAVIGKALLGVYPQL-EPEFVYGDYPHFH- G---HK---eNK ATAKK--F-FYSNIM-NFFKKD-- | 1041 |
| WP_019805234 | 982 | HHAHDAYLNAVIGKALLGVYPQL-EPEFVYGDYPHFH- G---HK---eNK ATAKK--F-FYSNIM-NFFKKD-- | 1041 |
| WP_024783594 | 982 | HHAHDAYLNAVIGKALLGVYPQL-EPEFVYGDYPHFH- G---HK---eNK ATAKK--F-FYSNIM-NFFKKD-- | 1041 |
| WP_024784288 | 984 | HHAHDAYLNAVIGKALLGVYPQL-EPEFVYGDYPHFH- S---YR---eRK ATQKM--F-FYSNIM-NMFKSKVK | 1046 |
| WP_024784666 | 982 | HHAHDAYLNAVIGKALLGVYPQL-EPEFVYGDYPHFH- G---HK---eNK ATAKK--F-FYSNIM-NFFKKD-- | 1041 |
| WP_024784894 | 982 | HHAHDAYLNAVIGKALLGVYPQL-EPEFVYGDYPHFH- G---HK---eNK ATAKK--F-FYSNIM-NFFKKD-- | 1041 |
| WP_024786433 | 984 | HHAHDAYLNAVIGKALLGVYPQL-EPEFVYGDYPHFH- S---YR---eRK ATQKM--F-FYSNIM-NMFKSKVK | 1046 |
| WP_049473442 | 982 | HHAHDAYLNAVIGKALLGVYPQL-EPEFVYGDYPHFH- G---HE---eNK ATAKK--F-FYSNIM-NFFKKD-- | 1041 |
| WP_049474547 | 982 | HHAHDAYLNAVIGKALLGVYPQL-EPEFVYGDYPHFH- G---HK---eNK ATAKK--F-FYSNIM-NFFKKD-- | 1041 |
| EMC03581 | 975 | HHAHDAYLNAVIGKALLGVYPQL-EPEFVYGDYPHFH- G---HK---eNK ATAKK--F-FYSNIM-NFFKKD-- | 1034 |
| WP_000428612 | 987 | HHAHDAYLNAVVAKAILKKYPKL-EPEFVYGDYQKYDL SkdpKEI---EK ATEKY--F-FYSNLL-NFFKEEVH | 1058 |
| WP_000428613 | 985 | HHAHDAYLNAVVAKAILKKYPKL-EPEFVYGDYQKYDL SrnpKEV---EK ATEKY--F-FYSNLL-NFFKEEVH | 1056 |
| WP_049523028 | 980 | HHAHDAYLNAVVAKAILKKYPKL-EPEFVYGDYQKYDL TkdpKEI---EK ATEKY--F-FYSNLL-NFFKDKVY | 1051 |
| WP_003107102 | 951 | HHAHDAYLNAVVGTALLKKYPKL-EAEFVYGDYKHYDL S---DTSl--GK ATAKM--F-FYSNIM-NFFKKEVR | 1020 |
| WP_054279288 | 983 | HHAHDAYLNAVVGTALLKKYPKL-ASEFVYGDYQKYDL S---GKAs--GK ATAKY--F-FYSNLM-NFFKSEVK | 1052 |
| WP_049531101 | 985 | HHAHDAYLNAVVAKAILKKYPKL-EPEFVYGDYQKYDL SrdpKEI---EK ATEKY--F-FYSNLL-NFFKEEVH | 1056 |
| WP_049538452 | 985 | HHAHDAYLNAVVAKAILKKYPKL-EPEFVYGDYQKYDL SkdpKDI---EK ATEKY--F-FYSNLL-NFFKEEVH | 1056 |
| WP_049549711 | 987 | HHAHDAYLNAVVAKAILKKYPKL-EPEFVYGDYQKNDL SkdpKDI---EK ATEKY--F-FYSNLL-NFFKEEVH | 1058 |
| WP_007896501 | 988 | HHAHDAYLNAVVGTALLKKYPKL-EAEFVYGDYKHFDL S---DPSl--GK ATAKV--F-FYSNIM-NFFKEELS | 1057 |
| EFR44625 | 940 | HHAHDAYLNAVVGTALLKKYPKL-EAEFVYGDYKHFDL S---DPSl--GK ATAKV--F-FYSNIM-NFFKEELS | 1009 |
| WP_002897477 | 984 | HHAHDAYLNAVVAKAILKKYPKL-EPEFVYGDYQKYDL FkpsKEI---EK ATEKY--F-FYSNLL-NFFKEEVL | 1055 |
| WP_002906454 | 984 | HHAHDAYLNAVVAKAILKKYPKL-EPEFVYGDYQKYDL SkasNTI---EK ATEKY--F-FYSNLL-NFFKEEVR | 1055 |
| WP_009729476 | 985 | HHAHDAYLNAVVAKAILKKYPKL-EPEFVYGDYQKYDL SkdpKEI---EK ATEKY--F-FYSNLL-NFFKEEVH | 1056 |
| CQR24647 | 984 | HHAHDAYLNAVVAKALLIRYPKL-EPEFVYGEYPKYN- S---YRE---EK ATEKM--F-FYSNIM-NMFKTTIK | 1046 |
| WP_000066813 | 989 | HHAHDAYLNAVLAKAILKKYPKL-EPEFVYGDYQKYDL SrepKEV---EK ATQKY--F-FYSNLL-NFFKEEVH | 1060 |
| WP_009754323 | 985 | HHAHDAYLNAVVAKAILKKYPKL-EPEFVYGDYQKYDL SkdpKEI---EK ATEKY--F-FYSNLL-NFFKEEVH | 1056 |
| WP_044674937 | 986 | HHAHDAYLNAVVATALLKKYPQL-APEFVYGDYPKYN- S---YKS---EK ATEKV--L-FYSNIM-NFFRRVLV | 1048 |
| WP_044676715 | 988 | HHAHDAYLNAVVATALLKKYPQL-APEFVYGDYPKYN- S---YKS---EK ATEKV--L-FYSNIM-NFFRRVLV | 1050 |
| WP_044680361 | 988 | HHAHDAYLNAVVATALLKKYPQL-APEFVYGDYPKYN- S---YKS---EK ATEKV--L-FYSNIM-NFFRRVLV | 1050 |
| WP_044681799 | 986 | HHAHDAYLNAVVATALLKKYPQL-APEFVYGDYPKYN- S---YKS---EK ATEKV--L-FYSNIM-NFFRRVLV | 1048 |
| WP_049533112 | 981 | HHAHDAYLNAVIGTALLKKYPKL-ASEFVYGEFKKYDV S---DK---eIG KATAK--YfFYSNLM-NFFKKEVK | 1050 |
| WP_029090905 | 952 | HHAQDAFLVAFLGTNITSNYPKI-EMEYLFKGYQHYLN ------Ev--GK AAKPKftF-IVENLS--------- | 1007 |
| WP_006506696 | 977 | HHAHDAYIVALIGGFMRDRYPNMhDSKAVYSEYMKMGR ----NKNd--QK -----G---FVINSM-NYPY-EV- | 1038 |
| AIT42264 | 982 | HHAHDAYLNAVVGTALIKKYPKL-ESEFVYGDYKVYDV S---EQEi--GK ATAKY--F-FYSNIM-NFFKTEIT | 1051 |
| WP_034440723 | 981 | HHAHDAYLNGVIALKLLELYPYM-AKDLIYGKYSYHRK G---------DK ATQAK--Y-KMSNII-ERFSQDL- | 1041 |
| AKQ21048 | 982 | HHAHDAYLNAVVGTALIKKYPKL-ESEFVYGDYKVYDV S---EQEi--GK ATAKY--F-FYSNIM-NFFKTEIT | 1051 |
| WP_004636532 | 981 | HHGHDAYLNAVVATTIMKVYPNL-KPQFVYGQYKKTSM ----FKE---EK ATARK--H-FYSNIT-KFFKKEKV | 1042 |
| WP_002364836 | 988 | HHGQDAYLNCVVATTLLKVYPNL-APEFVYGEYPKFQT ----FKE---EK ATAKA--I-IYTNLL-RFFTED-- | 1047 |
| WP_016631044 | 939 | HHGQDAYLNCVVATTLLKVYPNL-APEFVYGEYPKFQT ----FKE---NK ATAKA--I-IYTNLL-RFFTED-- | 998 |
| EMS75795 | 723 | HHAHDAYLNGVVAIALLKKYPKL-EPEFVYGNYTKENL ----AT---eNK ATAKK--E-FYSNIL-RFFEKE-- | 782 |
| WP_002373311 | 988 | HHGQDAYLNCVVATTLLKVYPNL-APEFVYGEYPKFQA ----FKE---NK ATAKT--I-IYTNLM-RFFTED-- | 1047 |
| WP_002378009 | 988 | HHGQDAYLNCVVATTLLKVYPNL-APEFVYGEYPKFQT ----FKE---NK ATAKA--I-IYTNLL-RFFTED-- | 1047 |
| WP_002407324 | 988 | HHGQDAYLNCVVATTLLKVYPNL-APEFVYGEYPKFQT ----FKE---NK ATAKA--I-IYTNLL-RFFTED-- | 1047 |
| WP_002413717 | 988 | HHGQDAYLNCVVATTLLKVYPNL-APEFVYGEYPKFQT ----FKE---NK ATAKA--I-IYTNLL-RFFTED-- | 1047 |
| WP_010775580 | 990 | HHGQDAYLNCVVATTLLKVYPNL-APEFVYGEYPKFQA ----FKE---NK ATAKA--I-IYTNLL-RFFTED-- | 1049 |
| WP_010818269 | 988 | HHGQDAYLNCVVATTLLKVYPNL-APEFVYGEYPKFQT ----FKE---NK ATAKA--I-IYTNLL-RFFTED-- | 1047 |
| WP_010824395 | 988 | HHGQDAYLNCVVATTLLKVYPNL-APEFVYGEYPKFQT ----FKE---NK ATAKT--I-IYTNLM-RFFTED-- | 1047 |
| WP_016622645 | 988 | HHGQDAYLNCVVATTLLKVYPNL-APEFVYGEYPKFQT ----FKE---NK ATAKA--I-IYTNLL-RFFTED-- | 1047 |
| WP_033624816 | 988 | HHGQDAYLNCVVATTLLKVYPNL-APEFVYGEYPKFQA ----FKE---NK AMAKA--I-IYTNLL-RFFTED-- | 1047 |
| WP_033625576 | 988 | HHGQDAYLNCVVATTLLKVYPNL-APEFVYGEYPKFQA ----FKE---NK ATAKA--I-IYTNLM-RFFTEV-- | 1047 |
| WP_033789179 | 988 | HHGQDAYLNCVVATTLLKVYPNL-APEFVYGEYPKFQA ----FKE---NK ATAKA--I-IYTNLL-RFFTED-- | 1047 |
| WP_002310644 | 989 | HHAHDAYLNGVVALALLKKYPQL-APEFVYGEYLKFNA ----HK---aNK ATVKK--E-FYSNIM-KFFESD-- | 1048 |
| WP_002312694 | 990 | HHAHDAYLNGVVALALLKKYPQL-APEFVYGEYLKFNA ----HK---aNK ATVKK--E-FYSNIM-KFFESD-- | 1049 |
| WP_002314015 | 990 | HHAHDAYLNGVVALALLKKYPQL-APEFVYGEYLKFNA ----HK---aNK ATVKK--E-FYSNIM-KFFESD-- | 1049 |
| WP_002320716 | 990 | HHAHDAYLNGVVALALLKKYPQL-APEFVYGEYLKFNA ----HK---aNK ATVKK--E-FYSNIM-KFFESD-- | 1049 |
| WP_002330729 | 989 | HHAHDAYLNGVVALALLKKYPQL-APEFVYGEYLKFNA ----HK---aNK ATVKK--E-FYSNIM-KFFESD-- | 1048 |
| WP_002335161 | 990 | HHAHDAYLNGVVALALLKKYPQL-APEFVYGEYLKFNA ----HK---aNK ATVKK--E-FYSNIM-KFFESD-- | 1049 |
| WP_002345439 | 990 | HHAHDAYLNGVVALALLKKYPQL-APEFVYGEYLKFNA ----HK---aNK ATVKK--E-FYSNIM-KFFESD-- | 1049 |
| WP_034867970 | 980 | HHAHDAYLNGFIANVLLKRYPKL-APEFVYGKYVKYSL ----AR---eNK ATAKK--E-FYSNIL-KFLESD-- | 1039 |
| WP_047937432 | 990 | HHAHDAYLNGVIALALLKKYPQL-APEFVYGEYLKFNA ----HK---aNK ATVKK--E-FYSNIM-KFFESD-- | 1049 |
| WP_010720994 | 980 | HHAHDAYLNGFIANVLLKRYPKL-APEFVYGKYVKYSL ----AR---eNK ATAKK--E-FYSNIL-KFLESD-- | 1039 |
| WP_010737004 | 980 | HHAHDAYLNGFIANVLLKRYPKL-APEFVYGKYVKYSL ----AR---eNK ATAKK--E-FYSNIL-KFLESD-- | 1039 |
| WP_034700478 | 980 | HHAHDAYLNGFIANVLLKRYPKL-APEFVYGKYVKYSL ----AR---eNK ATAKK--E-FYSNIL-KFLESD-- | 1039 |
| WP_007209003 | 978 | HHAHDAYLNAVVALSLLRVYPQL-KPEFVYGEYGKNS- ----IHDq--NK ATIKK---qFYSNIT-RYFASK-- | 1037 |
| WP_023519017 | 974 | HHAHDAYLNGVVAMTLLKKYPKL-APEFVYGSYIKGDI ----NQ---iNK ATAKK--E-FYSNIM-KFFESE-- | 1033 |
| WP_010770040 | 986 | HHGHDAYLNGVVANSLLRVYPQL-QPEFVYGDYPKENA ----YKA---NK ATAKK--Q-LYTNIM-KFFAED-- | 1045 |
| WP_048604708 | 983 | HHAHDAYLNGVVATALLKIYPOL-EPEFVYGEFHRFNA ----FKE---NK ATAKK--Q-FYSNLM-EFSKSD-- | 1042 |
| WP_010750235 | 983 | HHAHDAYLNAVVALALLKKYPRL-APEFVYGSFAKFHL ----VK---eNK ATAKK--E-FYSNIL-KFFEKE-- | 1042 |
| AII16583 | 1021 | HHAHDAYLNAVVGTALIKKYPKL-ESEFVYGDYKVYDV S---EQEi--GK ATAKY--F-FYSNIM-NFFKTEIT | 1090 |
| WP_029073316 | 988 | HHAHDAYIATILGTYIGHRFESL-DAKYIYGEYQKIFR ----NKNk--DK ---KDg---FILNSM-RNLYADK- | .052 |
| WP_031589969 | 988 | HHAHDAYIATILGTYIGHRFESL-DAKYIYGEYKRIFR ----QKNk--GK ---NDg---FILNSM-RNIYADK- | 1052 |
| KDA45870 | 973 | HHAHDAYLNAVVANLIMAKYPEL-EPEFVYGKYRKTK- ----FKG1--GK ATAKN---tLYANVL-YELKENEV | 1034 |
| WP_039099354 | 991 | HHAFDAYLTAFVGLYLLKRYPKL-KPYFVYGEYQKAS- ----QQ----DK ---RN--F----NFL-NGLKKD-- | 1043 |
| AKP02966 | 1000 | HHAQDAYLASFLGTYRLRRFPTD-EMLLMNGEYNKFYG -----KElysKK -SRKN-gF-IISPLV--------- | 1062 |
| WP_010991369 | 985 | HHAHDAYLNGVVANTLLKVYPQL-EPEFVYGDYHQFDW ----FKA---NK ATAKK--Q-FYTNIM-LFFAQK-- | 1044 |
| WP_033838504 | 985 | HHAHDAYLNGVVANTLLKVYPQL-EPEFVYGDYHQFDW ----FKA---NK ATAKK--Q-FYTNIM-LFFAQK-- | 1044 |
| EHN60060 | 988 | HHAHDAYLNGVVANTLLKVYPQL-EPEFVYGDYHQFDW ----FKA---NK ATAKK--Q-FYTNIM-LFFAQK-- | 1047 |
| EFR89594 | 754 | HHAHDAYLNGVVANTLLKVYPQL-EPEFVYGDYHQFDW ----FKA---NK ATAKK--Q-FYTNIM-LFFAQK-- | 813 |
| WP_038409211 | 985 | HHAHDAYLNGVVANTLLKVYPOL-EPEFVYGDYHOFDW ----FKA---NK ATAKK--Q-FYTNIM-RFFAKE-- | 1044 |
| EFR95520 | 604 | HHAHDAYLNGVVANTLLKVYPQL-EPEFVYGDYHOFDW ----FKA---NK ATAKK--Q-FYTNIM-RFFAKE-- | 663 |
| WP_003723650 | 985 | HHAHDAYLNGVVANTLLKVYPQL-EPEFVYGEYHQFDW ----FKA---NK ATAKK--Q-FYTNIM-LFFAQK-- | 1044 |
| WP_003727705 | 985 | HHAHDAYLNGVVANTLLKVYPOL-EPEFVYGEYHQFDW ----FKA---NK ATAKK--Q-FYTNIM-LFFAQK-- | 1044 |
| WP_003730785 | 985 | HHAHDAYLNGVVANTLLKVYPQL-EPEFVYGEYHQFDW ----FKA---NK ATAKK--Q-FYTNIM-LFFAQK-- | 1044 |
| WP_003733029 | 985 | HHAHDAYLNGVVANTLLKVYPQL-EPEFVYGEYHQFGW ----FKA---NK ATAKK--Q-FYTNIM-LFFAQK-- | 1044 |
| WP_003739838 | 985 | HHAHDAYLNGVVANTLLKVYPQL-EPEFVYGEYHQFDW ----FKA---NK ATAKK--Q-FYTNIM-LFFAQK-- | 1044 |
| WP_014601172 | 985 | HHAHDAYLNGVVANTLLKVYPOL-EPEFVYGEYHQFDW ----FKA---NK ATAKK--Q-FYTNIM-LFFGQK-- | 1044 |
| WP_023548323 | 985 | HHAHDAYLNGVVANTLLKVYPQL-EPEFVYGEYHQFDW ----FKA---NK ATAKK--Q-FYTNIM-LFFAQK-- | 1044 |
| WP_031665337 | 985 | HHAHDAYLNGVVANTLLKVYPQL-EPEFVYGEYHQFDW ----FKA---NK ATAKK--Q-FYTNIM-LFFAQK-- | 1044 |
| WP_031669209 | 985 | HHAHDAYLNGVVANTLLKVYPQL-EPEFVYGEYHQFDW ----FKA---NK ATAKK--Q-FYTNIM-LFFAQK-- | 1044 |
| WP_033920898 | 985 | HHAHDAYLNGVVANTLLKVYPQL-EPEFVYGEYHQFDW ----FKA---NK ATAKK--Q-FYTNIM-LFFAQK-- | 1044 |
| AKI42028 | 988 | HHAHDAYLNGVVANTLLKVYPQL-EPEFVYGEYHQFDW ----FKA---NK ATAKK--Q-FYTNIM-LFFGQK-- | 1047 |
| AKI50529 | 988 | HHAHDAYLNGVVANTLLKVYPQL-EPEFVYGEYHQFDW ----FKA---NK ATAKK--Q-FYTNIM-LFFAQK-- | 1047 |
| EFR83390 | 433 | HHAHDAYLNGVVANTLLKVYPQL-EPEFVYGEYHQFDW ----FKA---NK ATAKK--Q-FYTNIM-LFFAQK-- | 492 |
| WP_046323366 | 985 | HHAHDAYLNCVVANTLLKVYPQL-EPEFVYGDYHQFDW ----FKA---NK ATAKK--Q-FYTNIM-LFFAKK-- | 1044 |
| AKE81011 | 998 | HHAHDAYLNAVVGTALIKKYPKL-ESEFVYGDYKVYDV S---EQEi--GK ATAKY--F-FYSNIM-NFFKTEIT | 1067 |
| CUO82355 | 981 | HHAHDAYIVALIGGFMRDRYPNMhDSKAVYSEYMKMER ----NKNd--QK -----g---FVINSM-NYPY-EV- | 1042 |
| WP_033162887 | 983 | HHAHDAYIACIVGQFMHONFEHL-DAKIIYGQYK---- -----KNy--KK ---NYg---FILNSM-NHLQSDI- | 1042 |
| AGZ01981 | 1015 | HHAHDAYLNAVVGTALIKKYPKL-ESEFVYGDYKVYDV S---EQEi--GK ATAKY--F-FYSNIM-NFFKTEIT | 1084 |
| AKA60242 | 982 | HHAHDAYLNAVVGTALIKKYPKL-ESEFVYGDYKVYDV S---EQEi--GK ATAKY--F-FYSNIM-NFFKTEIT | 1051 |
| AKS40380 | 982 | HHAHDAYLNAVVGTALIKKYPKL-ESEFVYGDYKVYDV S---EQEi--GK ATAKY--F-FYSNIM-NFFKTEIT | 1051 |
| 4UN5_B | 986 | HHAHDAYLNAVVGTALIKKYPKL-ESEFVYGDYKVYDV S---EQEi--GK ATAKY--F-FYSNIM-NFFKTEIT | 1055 |
| WP_010922251 | 1052 | 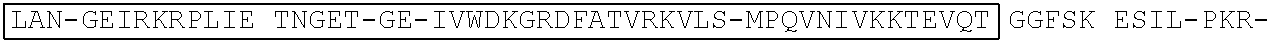 | 1114 |
| WP_039695303 | 1059 | YAD-GTVFERPIIE T-NAD-GE-IAWNKQIDFEKVRKVLS-YPQVNIVKKVETQT GGFSK ESIL-PKG- | 1120 |
| WP_045635197 | 1056 | YAD-GTIVKRENIE Y-SKDtGE-IAWNKEKDFAIIKKVLS-LPQVNIVKKREVQT GGFSK ESIL-PKG- | 1118 |
| 5AXW_A | 772 | YKYSHRVDKKPNRE VNNLN-GL---YDKDND--KLKKLINKSPEKLLMYHHDPQT --YQK KLIMeQYGd | 852 |
| WP_009880683 | 736 | LAN-GEIRKRPLIE TNGET-GE-IVWDKGRDFATVRKVLS-MPQVNIVKKTEVQT GGFSK ESIL-PKR- | 798 |
| WP_010922251 | 1052 | LAN-GEIRKRPLIE TNGET-GE-IVWDKGRDFATVRKVLS-MPQVNIVKKTEVQT GGFSK ESIL-PKR- | 1114 |
| WP_011054416 | 1052 | LAN-GEIRKRPLIE TNGET-GE-IVWDKGRDFATVRKVLS-MPQVNIVKKTEVQT GGFSK ESIL-PKR- | 1114 |
| WP_011284745 | 1052 | LAN-GEIRKRPLIE TNGET-GE-IVWDKGRDFATVRKVLS-MPQVNIVKKTEVQT GGFSK ESIL-PKR- | 1114 |
| WP_011285506 | 1052 | LAN-GEIRKRPLIE TNGET-GE-IVWDKGRDFATVRKVLS-MPQVNIVKKTEVQT GGFSK ESIL-PKR- | 1114 |
| WP_011527619 | 1052 | LAN-GEIRKRPLIE TNGET-GE-IVWDKGRDFATVRKVLS-MPQVNIVKKTEVQT GGFSK ESIL-PKR- | 1114 |
| WP_012560673 | 1052 | LAN-GEIRKRPLIE TNGET-GE-IVWDKGRDFATVRKVLS-MPQVNIVKKTEVQT GGFSK ESIL-PKR- | 1114 |
| WP_014407541 | 1051 | LAN-GEIRKRPLIE TNGET-GE-IVWDKGRDFATVRKVLS-MPQVNIVKKTEVQT GGFSK ESIL-PKR- | 1113 |
| WP_020905136 | 1052 | LAN-GEIRKRPLIE TNGET-GE-IVWDKGRDFATVRKVLS-MPQVNIVKKTEVQT GGFSK ESIL-PKR- | 1114 |
| WP_023080005 | 1051 | LAN-GEIRKRPLIE TNGET-GE-IVWDKGRDFATVRKVLS-MPQVNIVKKTEVQT GGFSK ESIL-PKR- | 1113 |
| WP_023610282 | 1051 | LAN-GEIRKRPLIE TNGET-GE-IVWDKGRDFATVRKVLS-MPQVNIVKKTEVQT GGFSK ESIL-PKR- | 1113 |
| WP_030125963 | 1052 | LAN-GEIRKRPLIE TNGET-GE-IVWDKGRDFATVRKVLS-MPQVNIVKKTEVQT GGFSK ESIL-PKR- | 1114 |
| WP_030126706 | 1052 | LAN-GEIRKRPLIE TNGET-GE-IVWDKGRDFATVRKVLS-MPQVNIVKKTEVQT GGFSK ESIL-PKR- | 1114 |
| WP_031488318 | 1052 | LAN-GEIRKRPLIE TNGET-GE-IVWDKGRDFATVRKVLS-MPQVNIVKKTEVQT GGFSK ESIL-PKR- | 1114 |
| WP_032460140 | 1052 | LAN-GEIRKRPLIE TNGET-GE-IVWDKGRDFATVRKVLS-MPQVNIVKKTEVQT GGFSK ESIL-PKR- | 1114 |
| WP_032461047 | 1052 | LAN-GEIRKRPLIE TNGET-GE-IVWDKGRDFATVRKVLS-MPQVNIVKKTEVQT GGFSK ESIL-PKR- | 1114 |
| WP_032462016 | 1052 | LAN-GEIRKRPLIE TNGET-GE-IVWDKGRDFATVRKVLS-MPQVNIVKKTEVQT GGFSK ESIL-PKR- | 1114 |
| WP_032462936 | 1052 | LAN-GEIRKRPLIE TNGET-GE-IVWDKGRDFATVRKVLS-MPQVNIVKKTEVQT GGFSK ESIL-PKR- | 1114 |
| WP_032464890 | 1052 | LAN-GEIRKRPLIE TNGET-GE-IVWDKGRDFATVRKVLS-MPQVNIVKKTEVQT GGFSK ESIL-PKR- | 1114 |
| WP_033888930 | 877 | LAN-GEIRKRPLIE TNGET-GE-IVWDKGRDFATVRKVLS-MPQVNIVKKTEVQT GGFSK ESIL-PKR- | 939 |
| WP_038431314 | 1052 | LAN-GEIRKRPLIE TNGET-GE-IVWDKGRDFATVRKVLS-MPQVNIVKKTEVQT GGFSK ESIL-PKR- | 1114 |
| WP_038432938 | 1051 | LAN-GEIRKRPLIE TNGET-GE-IVWDKGRDFATVRKVLS-MPQVNIVKKTEVQT GGFSK ESIL-PKR- | 1113 |
| WP_038434062 | 1052 | LAN-GEIRKRPLIE TNGET-GE-IVWDKGRDFATVRKVLS-MPQVNIVKKTEVQT GGFSK ESIL-PKR- | 1114 |
| BAQ51233 | 963 | LAN-GEIRKRPLIE TNGET-GE-IVWDKGRDFATVRKVLS-MPQVNIVKKTEVQT GGFSK ESIL-PKR- | 1025 |
| KGE60162 | 227 | LAN-GEIRKRPLIE TNGET-GE-IVWDKGRDFATVRKVLS-MPQVNIVKKTEVQT GGFSK ESIL-PKR- | 289 |
| KGE60856 | 1 | ------------IE TNGET-GE-IVWDKGRDFATVRKVLS-MPQVNIVKKTEVQT GGFSK ESIL-PKR- | 52 |
| WP_002989955 | 1052 | LAN-GEIRKRPLIE TNGET-GE-IVWDKGRDFATVRKVLS-MPQVNIVKKTEVQT GGFSK ESIL-PKR- | 1114 |
| WP_003030002 | 1042 | -----------DIQ T-NED-GE-IAWNKEKHIKILRKVLS-YPQVNIVKKTEEQT GGFSK ESIL-PKG- | 1093 |
| WP_003065552 | 1059 | YSN-GKVIVRPVVE Y-SKD-TEdIAWDKKSNFRTICKVLS-YPQVNIVKKVETOT GGFSK ESIL-PKG- | 1121 |
| WP_001040076 | 1050 | LAD-GTVVVKDDIE VNNDT-GE-IVWDKKKHFATVRKVLS-YPQVNIVKKTEIQT GGFSK ESIL-AHG- | 1112 |
| WP_001040078 | 1058 | LAD-GSIVVRPVIE TGRYM-GK-TAWDKKKHFATVRKVLS-YPQVNIVKKTEIQT GGFSK ESIL-AHG- | 1120 |
| WP_001040080 | 1050 | LAD-GTVVVKDDIE VNNDT-GE-IVWDKKKHFATVRKVLS-YPONNIVKKTEIQT GGFSK ESIL-AHG- | 1112 |
| WP_001040081 | 1050 | LAD-GTVVVKDDIE VNNDT-GE-IVWDKKKHFATVRKVLS-YPQNNIVKKTEIQT GGFSK ESIL-AHG- | 1112 |
| WP_001040083 | 1050 | LAD-GTVVVKDDIE VNNDT-GE-IVWDKKKHFATVRKVLS-YPONNIVKKTEIQT GGFSK ESIL-AHG- | 1112 |
| WP_001040085 | 1050 | LAD-GTVVVKDDIE VNNDT-GE-IVWDKKKHFATVRKVLS-YPONNIVKKTEIQT GGFSK ESIL-AHG- | 1112 |
| WP_001040087 | 1050 | LAD-GTVVVKDDIE VNNDT-GE-IVWDKKKHFATVRKVLS-YPQNNIVKKTEIQT GGFSK ESIL-AHG- | 1112 |
| WP_001040088 | 1050 | LAD-GTVVVKDDIE VNNDT-GE-IVWDKKKHFATVRKVLS-YPQNNIVKKTEIQT GGFSK ESIL-AHG- | 1112 |
| WP_001040089 | 1050 | LAD-GTVVVKDDIE VNNDT-GE-IVWDKKKHFATVRKVLS-YPONNIVKKTEIQT GGFSK ESIL-AHG- | 1112 |
| WP_001040090 | 1050 | LAD-GTVVVKDDIE VNNDT-GE-IVWDKKKHFATVRKVLS-YPQNNIVKKTEIQT GGFSK ESIL-AHG- | 1112 |
| WP_001040091 | 1050 | LAD-GTVVVKDDIE VNNDT-GE-IVWDKKKHFATVRKVLS-YPQNNIVKKTEIQT GGFSK ESIL-AHG- | 1112 |
| WP_001040092 | 1050 | LAD-ETVVVKDDIE VNNET-GE-IAWDKKKHFATVRKVLS-YPQVNIVKKTEVQT GGFSK ESIL-AHS- | 1112 |
| WP_001040094 | 1050 | LAD-GTVVVKDDIE VNNDT-GE-IVWDKKKHFATVRKVLS-YPQVNIVKKTEIQT GGFSK ESIL-AHG- | 1112 |
| WP_001040095 | 1050 | LAD-GTVVVKDDIE VNNDT-GE-IVWDKKKHFATVRKVLS-YPQVNIVKKTEVQT GGFSK ESIL-AHG- | 1112 |
| WP_001040096 | 1050 | LAD-GTVVVKDDIE VNNDT-GE-IVWDKKKHFATVRKVLS-YPQVNIVKKTEVQT GGFSK ESIL-AHG- | 1112 |
| WP_001040097 | 1050 | LAD-GTVVVKDDIE VNNDT-GE-IVWDKKKHFATVRKVLS-YPQVNIVKKTEIQT GGFSK ESIL-AHG- | 1112 |
| WP_001040098 | 1050 | LAD-GTVVVKDDIE VNNDT-GE-IVWDKKKHFATVRKVLS-YPQVNIVKKTEIQT GGFSK ESIL-AHG- | 1112 |
| WP_001040099 | 1050 | LAD-GTVVVKDDIE VNNDT-GE-IVWDKKKHFATVRKVLS-YPQVNIVKKTEIQT GGFSK ESIL-AHG- | 1112 |
| WP_001040100 | 1050 | LAD-GTVVVKDDIE VNNDT-GE-IVWDKKKHFATVRKVLS-YPQVNIVKKTEIQT GGFSK ESIL-AHG- | 1112 |
| WP_001040104 | 1050 | LAD-GTVVVKDDIE VNNDT-GE-IVWDKKKHFATVRKVLS-YPONNIVKKTEIQT GGFSK ESIL-AHG- | 1112 |
| WP_001040105 | 1050 | LAD-GTVVVKDDIE VNNDT-GE-IVWDKKKHFATVRKVLS-YPQNNIVKKTEIQT GGFSK ESIL-AHG- | 1112 |
| WP_001040106 | 1050 | LAD-GTVVVKDDIE VNNDT-GE-IVWDKKKHFATVRKVLS-YPQVNIVKKTEIQT GGFSK ESIL-AHG- | 1112 |
| WP_001040107 | 1050 | LAD-GTVVVKDDIE VNNDT-GE-IVWDKKKHFATVRKVLS-YPQVNIVKKTEIQT GGFSK ESIL-AHG- | 1112 |
| WP_001040108 | 1050 | LAD-GTVVVKDDIE VNNDT-GE-IVWDKKKHFATVRKVLS-YPQVNIVKKTEIQT GGFSK ESIL-AHG- | 1112 |
| WP_001040109 | 1050 | LAD-GTVVVKDDIE VNNDT-GE-IVWDKKKHFATVRKVLS-YPQVNIVKKTEIQT GGFSK ESIL-AHG- | 1112 |
| WP_001040110 | 1050 | LAD-GTVVVKDDIE VNNDT-GE-IVWDKKKHFATVRKVLS-YPQVNIVKKTEIQT GGFSK ESIL-AHG- | 1112 |
| WP_015058523 | 1050 | LAD-ETVVVKDDIE VNNET-GE-IAWDKKKHFATVRKVLS-YPQVNIVKKTEVQT GGFSK ESIL-AHS- | 1112 |
| WP_017643650 | 1050 | LAD-GTVVVKDDIE VNNDT-GE-IVWDKKKHFATVRKVLS-YPQVNIVKKTEIQT GGFSK ESIL-AHG- | 1112 |
| WP_017647151 | 1050 | LAD-GTVVVKDDIE VNNDT-GE-IVWDKKKHFATVRKVLS-YPQVNIVKKTEIQT GGFSK ESIL-AHG- | 1112 |
| WP_017648376 | 1050 | LAD-GTVVVKDDIE VNNDT-GE-IVWDKKKHFATVRKVLS-YPQVNIVKKTEIQT GGFSK ESIL-AHG- | 1112 |
| WP_017649527 | 1050 | LAD-GTVVVKDDIE VNNDT-GE-IVWDKKKHFATVRKVLS-YPONNIVKKTEIQT GGFSK ESIL-AHG- | 1112 |
| WP_017771611 | 1050 | LAD-GTVVVKDDIE VNNDT-GE-IVWDKKKHFATVRKVLS-YPQVNIVKKTEIQT GGFSK ESIL-AHG- | 1112 |
| WP_017771984 | 1050 | LAD-GTVVVKDDIE VNNDT-GE-IVWDKKKHFATVRKVLS-YPQNNIVKKTEIQT GGFSK ESIL-AHG- | 1112 |
| CFQ25032 | 1050 | LAD-GTVVVKDDIE VNNDT-GE-IVWDKKKHFATVRKVLS-YPQNNIVKKTEIQT GGFSK ESIL-AHG- | 1112 |
| CFV16040 | 1050 | LAD-GTVVVKDDIE VNNDT-GE-IVWDKKKHFATVRKVLS-YPONNIVKKTEIQT GGFSK ESIL-AHG- | 1112 |
| KLJ37842 | 1050 | LAD-GTVVVKDDIE VNNDT-GE-IVWDKKKHFATVRKVLS-YPQNNIVKKTEIQT GGFSK ESIL-AHG- | 1112 |
| KLJ72361 | 1050 | LAD-GTVVVKDDIE VNNDT-GE-IVWDKKKHFATVRKVLS-YPQNNIVKKTEIQT GGFSK ESIL-AHG- | 1112 |
| KLL20707 | 1064 | LAD-GTVVVKDDIE VNNDT-GE-IVWDKKKHFATVRKVLS-YPONNIVKKTEIQT GGFSK ESIL-AHG- | 1126 |
| KLL42645 | 1050 | LAD-GTVVVKDDIE VNNDT-GE-IVWDKKKHFATVRKVLS-YPQVNIVKKTEIQT GGFSK ESIL-AHG- | 1112 |
| WP_047207273 | 1050 | LAD-GTVVVKDDIE VNNDT-GE-IVWDKKKHFATVRKVLS-YPQNNIVKKTEIQT GGFSK ESIL-AHG- | 1112 |
| WP_047209694 | 1050 | LAD-GTVVVKDDIE VNNDT-GE-IVWDKKKHFATVRKVLS-YPQVNIVKKTEIQT GGFSK ESIL-AHG- | 1112 |
| WP_050198062 | 1050 | LAD-GTVVIKDDIE VNNDT-GE-IVWDKKKHFATVRKVLS-YPQNNIVKKTEIQT GGFSK ESIL-AHG- | 1112 |
| WP_050201642 | 1050 | LAD-GTVVVKDDIE VNNDT-GE-IVWDKKKHFATVRKVLS-YPQNNIVKKTEIQT GGFSK ESIL-AHG- | 1112 |
| WP_050204027 | 1050 | LAD-GTVVVKDDIE VNNDT-GE-IVWDKKKHFATVRKVLS-YPQVNIVKKTEIQT GGFSK ESIL-AHG- | 1112 |
| WP_050881965 | 1050 | LAD-GTVVVKDDIE VNNDT-GE-IVWDKKKHFATVRKVLS-YPONNIVKKTEIQT GGFSK ESIL-AHG- | 1112 |
| WP_050886065 | 1050 | LAD-GTVVVKDDIE VNNDT-GE-IVWDKKKHFATVRKVLS-YPQNNIVKKTEIQT GGFSK ESIL-AHG- | 1112 |
| AHN30376 | 1050 | LAD-ETVVVKDDIE VNNET-GE-IAWDKKKHFATVRKVLS-YPQVNIVKKTEVQT GGFSK ESIL-AHS- | 1112 |
| EAO78426 | 1050 | LAD-GTVVVKDDIE VNNDT-GE-IVWDKKKHFATVRKVLS-YPQNNIVKKTEIQT GGFSK ESIL-AHG- | 1112 |
| CCW42055 | 1050 | LAD-GTVVVKDDIE VNNDT-GE-IVWDKKKHFATVRKVLS-YPQNNIVKKTEIQT GGFSK ESIL-AHG- | 1112 |
| WP_003041502 | 1051 | FAD-GTVVERPDIE T-SED-GE-IAWNKQTDFKIVRKVLS-YPQVNIVKKTEVQT HGLDR PSPK-PKP- | 1122 |
| WP_037593752 | 1043 | -----------DIQ T-NED-GE-IAWNKEKHIKILRKVLS-YPQVNIVKKTEEQT GGFSK ESIL-PKG- | 1094 |
| WP_049516684 | 1043 | -----------DIQ T-NED-GE-IAWNKEKHIKILRKVLS-YPQVNIVKKTEEQT GGFSK ESIL-PKG- | 1094 |
| GAD46167 | 1042 | -----------DIQ T-NED-GE-IAWNKEKHIKILRKVLS-YPQVNIVKKTEEQT GGFSK ESIL-PKG- | 1093 |
| WP_018363470 | 1063 | YSN-GKVIVRPVVE Y-SKDtGE-IAWNKRTDFEKVRKVLS-YPQVNIVKKVETQT GGFSK ESIL-PKG- | 1125 |
| WP_003043819 | 1061 | LAN-GEIRKRPLIE TNGET-GE-VVWNKEKDFATVRKVLA-MPQVNIVKKTEVQT GGFSK ESIL-SKR- | 1123 |
| WP_006269658 | 1042 | -----------DIQ T-NED-GE-IAWNKEKHIKILRKVLS-YPQVNIVKKTEEQT GGFSK ESIL-PKG- | 1093 |
| WP_048800889 | 1052 | FAD-GTVVERPDIE T-SED-GE-IAWNKQTDFKIVRKVLS-YPQVNIVKKVEKQT GRFSK ESIL-PKG- | 1113 |
| WP_012767106 | 1051 | LAN-GEIRKRPLIE TNEET-GE-IVWDKGRDFATVRKVLS-MPQVNIVKKTEVQT GALTN ESIY-ARG- | 1113 |
| WP_014612333 | 1051 | LAN-GEIRKRPLIE TNEET-GE-IVWDKGRDFATVRKVLS-MPQVNIVKKTEVQT GALTN ESIY-ARG- | 1113 |
| WP_015017095 | 1051 | LAN-GEIRKRPLIE TNEET-GE-IVWNKGRDFATVRKVLS-MPQVNIVKKTEVQT GALTN ESIY-ARG- | 1113 |
| WP_015057649 | 1051 | LAN-GEIRKRPLIE TNEET-GE-IVWDKGRDFATVRKVLS-MPQVNIVKKTEVQT GALTN ESIY-ARG- | 1113 |
| WP_048327215 | 1051 | LAN-GEIRKRPLIE TNEET-GE-IVWDKGRDFATVRKVLS-MPQVNIVKKTEVQT GALTN ESIY-ARG- | 1113 |
| WP_049519324 | 1051 | LAN-GEIRKRPLIE TNEET-GE-IVWDKGRDFATVRKVLS-MPQVNIVKKTEVQT GALTN ESIY-ARG- | 1113 |
| WP_012515931 | 1044 | L------------H VNSD--GE-EIWNANKHLPIIKNVLS-IPQVNIVKKTEVQT GGFYK ESIL-SKG- | 1094 |
| WP_021320964 | 1044 | L------------H VNSD--GE-EIWNANKHLPIIKNVLS-IPQVNIVKKTEVQT GGFYK ESIL-SKG- | 1094 |
| WP_037581760 | 1044 | L------------H VNSD--GE-EIWNANKHLPIIKNVLS-IPQVNIVKKTEVQT GGFYK ESIL-SKG- | 1094 |
| WP_004232481 | 1062 | YAD-GRVFERPDIE T-NAD-GE-VVWNKQRDFNIVRKVLS-YPQVNIVKKVEVQT GGFSK ESIL-PKG- | 1123 |
| WP_009854540 | 1057 | YAD-GTVFERPIIE T-NAD-GE-IAWNKQIDFEKVRKVLS-YPQVNIVKKVETQT GGFSK ESIL-PKG- | 1118 |
| WP_012962174 | 1057 | YSN-GKVVVRPVIE C-SKDtGE-IAWNKQTDFEKVRRVLS-YPQVNIVKKVETQT GGFSK ESIL-PKG- | 1119 |
| WP_039695303 | 1059 | YAD-GTVFERPIIE T-NAD-GE-IAWNKQIDFEKVRKVLS-YPQVNIVKKVETQT GGFSK ESIL-PKG- | 1120 |
| WP_014334983 | 1062 | YAD-GRVFERPDIE T-NAD-GE-VVWNKQKDFDIVRKVLS-YPQVNIVKKVEAQT GGFSK ESIL-SKG- | 1123 |
| WP_003099269 | 1052 | LAD-DTIFTRPQIE VNTET-GE-IVWDKVKDMQTIRKVMS-YPQVNIVMKTEVQT GGFSK ESIW-PKG- | 1114 |
| AHY15608 | 1052 | LAD-DTIFTRPQIE VNTET-GE-IVWDKVKDMQTIRKVMS-YPQVNIVMKTEVQT GGFSK ESIW-PKG- | 1114 |
| AHY17476 | 1052 | LAD-DTIFTRPQIE VNTET-GE-IVWDKVKDMQTIRKVMS-YPQVNIVMKTEVQT GGFSK ESIW-PKG- | 1114 |
| ESR09100 | | -------------- ----------------------------------------- ----- --------- | |
| AGM98575 | 1052 | LAD-DTIFTRPQIE VNTET-GE-IVWDKVKDMQTIRKVMS-YPQVNIVMKTEVQT GGFSK ESIW-PKG- | 1114 |
| ALF27331 | 1042 | -----------DVR T-DKN-GE-IIWKKDEHISNIKKVLS-YPQVNIVKKVEEQT GGFSK ESIL-PKG- | 1093 |
| WP_018372492 | 1056 | --K----------K --DQEtGE-IVWDKKEIENIVKKVIY-SSPVNIVKKREEQS GALFK QSNM-AVGy | 1108 |
| WP_045618028 | 1057 | YAD-GTIVKRENIE Y-SKDtGE-IAWNKEKDFATIKKVLS-LPQVNIVKKTEEQT GGLFD NNIV-SKKk | 1124 |
| WP_045635197 | 1056 | YAD-GTIVKRENIE Y-SKDtGE-IAWNKEKDFAIIKKVLS-LPQVNIVKKREVQT GGFSK ESIL-PKG- | 1118 |
| WP_002263549 | 1042 | -----------DVR T-DKN-GE-IIWKKDEHISNIKKVLS-YPQVNIVKKVEEQT GGFSK ESIL-PKG- | 1093 |
| WP_002263887 | 1042 | -----------DVR T-DKN-GE-IIWKKDEHISNIKKVLS-YPQVNIVKKVEEQT GGFSK ESIL-PKG- | 1093 |
| WP_002264920 | 1042 | -----------DVR T-DKN-GE-IIWKKDEYISNIKKVLS-YPQVNIVKKVEEQT GGFSK ESIL-PKG- | 1093 |
| WP_002269043 | 1042 | -----------DVR T-DKN-GE-IIWKKDEHISNIKKVLS-YPQVNIVKKVEEQT GGFSK ESIL-PKG- | 1093 |
| WP_002269448 | 1042 | -----------DVR T-DKN-GE-IIWKKDEHISNIKKVLS-YPQVNIVKKVEEQT GGFSK ESIL-PKG- | 1093 |
| WP_002271977 | 1042 | -----------DVR T-DKN-GE-IIWKKDEHISNIKKVLS-YPQVNIVKKVEEQT GGFSK ESIL-PKG- | 1093 |
| WP_002272766 | 1042 | -----------DVR T-DKN-GE-IIWKKDEHISNIKKVLS-YPQVNIVKKVEEQT GGFSK ESIL-PKG- | 1093 |
| WP_002273241 | 1042 | -----------DVR T-DKN-GE-IIWKKDEHISNIKKVLS-YPQVNIVKKVEEQT GGFSK ESIL-PKG- | 1093 |
| WP_002275430 | 1042 | -----------DVR T-DKN-GE-IIWKKDEHISNIKKVLS-YPQVNIVKKVEEQT GGFSK ESIL-PKG- | 1093 |
| WP_002276448 | 1042 | -----------DVR T-DRN-GE-IIWKKDEHISNIKKVLS-YPQVNIVKKVEEQT GGFSK ESIL-PKG- | 1093 |
| WP_002277050 | 1047 | LAD-DQIVERPMIE VNDET-GE-IAWDKTKHITTVKKVLS-YPQVNIVKKVEEQT GGLED -----PKS- | 1111 |
| WP_002277364 | 1042 | -----------DVR T-DKN-GE-IIWKKDEHISNIKKVLS-YPQVNIVKKVEEQT GGFSK ESIL-PKG- | 1093 |
| WP_002279025 | 1042 | -----------DVR T-DKN-GE-IIWKKDEHISNIKKVLS-YPQVNIVKKVEEQT GGFSK ESIL-PKG- | 1093 |
| WP_002279859 | 1042 | -----------DVR T-DKN-GE-IIWKKDEYISNIKKVLS-YPQVNIVKKVEEQT GGFSK ESIL-PKG- | 1093 |
| WP_002280230 | 1042 | -----------DVR T-DKN-GE-IIWKKDEHISNIKKVLS-YPQVNIVKKVEEQT GGFSK ESIL-PKG- | 1093 |
| WP_002281696 | 1042 | -----------DVR T-DKN-GE-IIWKKDEHISNIKKVLS-YPQVNIVKKVEEQT GGFSK ESIL-PKG- | 1093 |
| WP_002282247 | 1047 | LAD-DQIVERPMIE VNDET-GE-IAWDKTKHITTVKKVLS-YPQVNIVKKVEEQT GGLFD -----PKS- | 1111 |
| WP_002282906 | 1042 | -----------DVR I-DKN-GE-IIWKKDEHISNIKKVLS-YPQVNIVKKVEEQT GGFSK ESIL-PKG- | 1093 |
| WP_002283846 | 1042 | -----------DVR T-DKN-GE-IIWKKDEHISNIKKVLS-YPQVNIVKKVEEQT GGFSK ESIL-PKG- | 1093 |
| WP_002287255 | 1042 | -----------DVR T-DKN-GE-IIWKKDEHISNIKKVLS-YPQVNIVKKVEEQT GGFSK ESIL-PKG- | 1093 |
| WP_002288990 | 1042 | -----------DVR T-DKN-GE-IIWKKDEHISNIKKVLS-YPQVNIVKKVEEQT GGFSK ESIL-PKG- | 1093 |
| WP_002289641 | 1042 | -----------DVR T-DKN-GE-IIWKKDEHISNIKKVLS-YPQVNIVKKVEEQT GGFSK ESIL-PKG- | 1093 |
| WP_002290427 | 1042 | -----------DVR T-DKN-GE-IIWKKDEYISNIKKVLS-YPQVNIVKKVEEQT GGFFK ESIL-PKG- | 1093 |
| WP_002295753 | 1042 | -----------DVR T-DKN-GE-IIWKKDEYISNIKKVLS-YPQVNIVKKVEEQT GGFSK ESIL-PKG- | 1093 |
| WP_002296423 | 1042 | -----------DVR T-DKN-GE-IIWKKDEHISNIKKVLS-YPQVNIVKKVEEQT GGFSK ESIL-PKG- | 1093 |
| WP_002304487 | 1056 | -----------DVR T-DKN-GE-IIWKKDEHISNIKKVLS-YPQVNIVKKVEEQT GGFSK ESIL-PKG- | 1107 |
| WP_002305844 | 1042 | -----------DVR T-DKN-GE-IIWKKDEHISNIKKVLS-YPQVNIVKKVEEQT GGFSK ESIL-PKG- | 1093 |
| WP_002307203 | 1042 | -----------DVR T-DKN-GE-IIWKKDEYISNIKKVLS-YPQVNIVKKVEEQT GGFSK ESIL-PKG- | 1093 |
| WP_002310390 | 1042 | -----------DVR T-DKN-GE-IIWKKDEYISNIKKVLS-YPQVNIVKKVEEQT GGFFK ESIL-PKG- | 1093 |
| WP_002352408 | 1042 | -----------DVR T-DKN-GE-IIWKKDEYISNIKKVLS-YPQVNIVKKVEEQT GGFSK ESIL-PKG- | 1093 |
| WP_012997688 | 1042 | -----------DVR T-DKN-GE-IIWKKDEHISNIKKVLS-YPQVNIVKKVEEQT GGFSK ESIL-PKG- | 1093 |
| WP_014677909 | 1042 | -----------DVR T-DKN-GE-IIWKKDEHISNIKKVLS-YPQVNIVKKVEEQT GGFSK ESIL-PKG- | 1093 |
| WP_019312892 | 1042 | -----------DVR T-DKN-GE-IIWKKDEHISNIKKVLS-YPQVNIVKKVEEQT GGFSK ESIL-PKG- | 1093 |
| WP_019313659 | 1042 | -----------DVR T-DKN-GE-IIWKKDEHISNIKKVLS-YPQVNIVKKVEEQT GGFSK ESIL-PKG- | 1093 |
| WP_019314093 | 1042 | -----------DVR T-DKN-GE-IIWKKDEYISNIKKVLS-YPQVNIVKKVEEQT GGFSK ESIL-PKG- | 1093 |
| WP_019315370 | 1042 | -----------DVR T-DKN-GE-IIWKKDEHISNIKKVLS-YPQVNIVKKVEEQT GGFSK ESIL-PKG- | 1093 |
| WP_019803776 | 1042 | -----------DVR T-DKN-GE-IIWKKDEHISNIKKVLS-YPQVNIVKKVEEQT GGFSK ESIL-PKG- | 1093 |
| WP_019805234 | 1042 | -----------DVR T-DKN-GE-IIWKKDEYISNIKKVLS-YPQVNIVKKVEEQT GGFFK ESIL-PKG- | 1093 |
| WP_024783594 | 1042 | -----------DVR T-DKN-GE-IIWKKDEHISNIKKVLS-YPQVNIVKKVEEQT GGFSK ESIL-PKG- | 1093 |
| WP_024784288 | 1047 | LAD-DQIVERPMIE VNDET-GE-IAWDKTKHITTVKKVLS-YPQVNIVKKVEEQT GGLED -----PKS- | 1111 |
| WP_024784666 | 1042 | -----------DVR T-DKN-GE-IIWKKDEHISNIKKVLS-YPQVNIVKKVEEQT GGFSK ESIL-PKG- | 1093 |
| WP_024784894 | 1042 | -----------DVR T-DKN-GE-IIWKKDEHISNIKKVLS-YPQVNIVKKVEEQT GGFSK ESIL-PKG- | 1093 |
| WP_024786433 | 1047 | LAD-DQIVERPMIE VNDET-GE-IAWDKTKHITTVKKVLS-YPQVNIVKKVEEQT GGLFD -----PKS- | 1111 |
| WP_049473442 | 1042 | -----------DVR T-DKN-GE-IIWKKDEHISNIKKVLS-YPQVNIVKKVEEQT GGFSK ESIL-PKG- | 1093 |
| WP_049474547 | 1042 | -----------DVR T-DKN-GE-IIWKKDEHISNIKKVLS-YPQVNIVKKVEEQT GGFSK ESIL-PKG- | 1093 |
| EMC03581 | 1035 | -----------DVR T-DKN-GE-IIWKKDEHISNIKKVLS-YPQVNIVKKVEEQT GGFSK ESIL-PKG- | 1086 |
| WP_000428612 | 1059 | YAD-GTIVKRENIE Y-SKDtGE-IAWNKEKDFATIKKVLS-LPQVNIVKKREVQT GGFSK ESIL-PKG- | 1121 |
| WP_000428613 | 1057 | YAD-GTIVKRENIE Y-SKDtGE-IAWNKEKDFATIKKVLS-YPQVNIVKKREVQT GGFSK ESIL-PKG- | 1119 |
| WP_049523028 | 1052 | YAD-GTIIQRGNVE Y-SKDtGE-IAWNKKRDFAIVRKVLS-YPQVNIVKKTEEQT GGFSK ESIL-PKG- | 1114 |
| WP_003107102 | 1021 | LAD-GTVITRPQIE TNTET-GE-IVWDKVKDIKTIRKVLS-IPQINVVKKTEVQT GGFSK ESIL-SKR- | 1083 |
| WP_054279288 | 1053 | LAN-GNIIKRSPIE VNEET-GE-IVWDKTKDFGTVRKVLS-APQVNIVKKTEIQT GGFSN ETIL-SKG- | 1115 |
| WP_049531101 | 1057 | YAD-GTIVKRENIE Y-SKDtGE-IAWNKEIDFATIRKILS-LSQVNIVKKTEEQT GGLFD NNIV-SKKk | 1124 |
| WP_049538452 | 1057 | YAD-GTIVKRENIE Y-SKDtGE-IAWNKEKDFATIKKILS-LPQVNIVKKTEEQT GGLFD NNIV-SKKk | 1124 |
| WP_049549711 | 1059 | YAD-GTIVKRENIE Y-SKDtGE-IAWNKEKDFATIKKVLS-YPQVNIVKKTEEQT GGLFD NNIV-SKEk | 1126 |
| WP_007896501 | 1058 | LAD-GTLMKRPVIE TNTET-GE-VVWDKVKDFKTIRKVLS-YPQVNIVKKTEIQS GAFSK ESVL-SKG- | 1120 |
| EFR44625 | 1010 | LAD-GTLMKRPVIE TNTET-GE-VVWDKVKDFKTIRKVLS-YPQVNIVKKTEIQS GAFSK ESVL-SKG- | 1072 |
| WP_002897477 | 1056 | YAD-GTIRKRENIE Y-SKDtGE-IAWDKEKDFATIKKVLS-YPQVNIVKKREVQT GGFSK ESIL-PKG- | 1118 |
| WP_002906454 | 1056 | YAD-GTIKKRENIE Y-SNDtGE-IAWNKEKDFATIKKVLS-LPQVNIVKKTEEQT GGLFD NNIV-SKKk | 1123 |
| WP_009729476 | 1057 | YAD-GTIVKRENIE Y-SKDtGE-IAWNKEKDFATIKKVLS-LPQVNIVKKREVQT GGFSK ESIL-PKG- | .119 |
| CQR24647 | 1047 | LAD-GRVVEKPVIE ANEET-GE-IAWDKTKHFANVKKVLS-YPQVSIVKKVEEQT GGFSK ESIL-PKG- | 1109 |
| WP_000066813 | 1061 | YAD-GTIVKRENIE Y-SKDtGE-IAWNKEKDFATVKKVLS-LPQVNIVKKTEVQT GGFSK ESIL-PKG- | 1123 |
| WP_009754323 | 1057 | YAD-GTIVKRENIE Y-SKDtGE-IAWNKEKDFVTIKKVLS-YPQVNIVKKREVQT GGFSK ESIL-PKG- | 1119 |
| WP_044674937 | 1049 | YSKtGEVRIRPVIE VNKET-GE-IVWDKKSDFRTVRKVLS-YPQVNVVKKVEMQT GGFSK ESIL-QHG- | 1112 |
| WP_044676715 | 1051 | YSKtGEVRIRPVIE VNKET-GE-IVWDKKSDFRTVRKVLS-YPQVNVVKKVEMQT GGFSK ESIL-QHG- | 1114 |
| WP_044680361 | 1051 | YSKtGEVRIRPVIE VNKET-GE-IVWDKKSDFRTVRKVLS-YPQVNVVKKVEMQT GGFSK ESIL-QHG- | 1114 |
| WP_044681799 | 1049 | YSKtGEVRIRPVIE VNKET-GE-IVWDKKSDFKTVRKVLS-YPQVNVVKKVEMQT GGFSK ESIL-QHG- | 1112 |
| WP_049533112 | 1051 | FAD-GTVVERPDIE T-SED-GE-IAWNKQTDFKIVRKVLS-YPQVNIVKKTEVQT HGLDR PSPK-PKP- | 1122 |
| WP_029090905 | 1008 | -KQ----------Q --NSTtGE-VKWNPEVDIAKLKRILN-FKQCNIVRKVEEQS GALFK ETIY-PVEe | 1061 |
| WP_006506696 | 1039 | --D----------- ------GK-LIWNP-DLINEIKKCFY-YKDCYCTTKLDOKS GQLEN -TVL-SNDa | 1084 |
| AIT42264 | 1052 | LAN-GEIRKRPLIE TNGET-GE-IVWDKGRDFATVRKVLS-MPQVNIVKKTEVQT GGFSK ESIL-PKR- | 1114 |
| WP_034440723 | 1042 | ------------LA --NPD-GE-IAWEKDKDLNTIRKVLS-SKQINIIKKAEEGK GRLFK ETIN-SRPS | 1092 |
| AKQ21048 | 1052 | LAN-GEIRKRPLIE TNGET-GE-IVWDKGRDFATVRKVLS-MPQVNIVKKTEVQT GGFSK ESIL-PKR- | 1114 |
| WP_004636532 | 1043 | -------------- VNEET-GE-ILWDTERHLSTIKRVLS-WKOMNIVKKVEKQK GQLWK ETIY-PKG- | 1092 |
| WP_002364836 | 1048 | --E----------P RFTKD-GE-ILWSN-SYLKTIKKELN-YHOMNIVKKVEVQK GGFSK ESIK-PKG- | 1098 |
| WP_016631044 | 999 | --E----------P RFTKD-GE-ILWSN-SYLKTIKKELN-YHOMNIVKKVEVOK GGFSK ESIK-PKG- | 1049 |
| EMS75795 | 783 | --E----------Y SYDEN-GE-IFWDKARHIPQIKKVIS-SHQVNIVKKVEVQT GGFYK ETVN-PKG- | 834 |
| WP_002373311 | 1048 | --E----------P RFTKD-SE-ILWSN-SYLKTIKKELN-YHOMNIVKKVEVQK GGFSK ESIK-PKG- | 1098 |
| WP_002378009 | 1048 | --E----------P RFTKD-GE-ILWSN-SYLKTIKKELN-YHOMNIVKKVEVQK GGFSK ESIK-PKG- | 1098 |
| WP_002407324 | 1048 | --E----------P RFTKD-GE-ILWSN-SYLKTIKKELN-YHOMNIVKKVEVQK GGFSK ESIK-PKG- | 1098 |
| WP_002413717 | 1048 | --E----------P RFTKD-GE-ILWSN-SYLKTIKKELN-YHQMNIVKKVEVQK GGFSK ESIK-PKG- | 1098 |
| WP_010775580 | 1050 | --E----------P RFTKD-GE-ILWSN-SYLKTIKKELN-YHOMNIVKKVEVQK GGFSK ESIK-PKG- | 1100 |
| WP_010818269 | 1048 | --E----------P RFTKD-GE-ILWSN-SYLKTIKKELN-YHOMNIVKKVEVQK GGFSK ESIK-PKG- | 1098 |
| WP_010824395 | 1048 | --E----------P RFTKD-GE-ILWSN-SYLKTIKKELN-YHQMNIVKKVEVQK GGFSK ESIK-PKG- | 1098 |
| WP_016622645 | 1048 | --E----------P RFTKD-GE-ILWSN-SYLKTIKKELN-YHQMNIVKKVEVQK GGFSK ESIK-PKG- | 1098 |
| WP_033624816 | 1048 | --E----------P RFTKD-GE-ILWSN-SYLKTIKKELN-YHOMNIVKKVEVOK GGFSK ESIK-PKG- | 1098 |
| WP_033625576 | 1048 | --E----------P RFTKD-GE-ILWSN-SYLKTIKKELN-YHOMNIVKKVEVOK GGFSK ESIK-PKG- | 1098 |
| WP_033789179 | 1048 | --E----------P RFTKD-GE-ILWSN-SYLKTIKKELN-YHQMNIVKKVEVQK GGFSK ESIK-PKG- | 1098 |
| WP_002310644 | 1049 | --T----------P VCDEN-GE-IFWDKSKSIAQVKKVIN-HHHMNIVKKTEIQK GGFSK ETVE-PKK- | 1100 |
| WP_002312694 | 1050 | --T----------P VCDEN-GE-IFWDKSKSIAQVKKVIN-HHHMNIVKKTEIQK GGFSK ETVE-PKK- | 1101 |
| WP_002314015 | 1050 | --T----------P VCDEN-GE-IFWDKSKSIAQVKKVIN-HHHMNIVKKTEIQK GGFSK ETVE-PKK- | 1101 |
| WP_002320716 | 1050 | --T----------P VCDEN-GE-IFWDKSKSIAQVKKVIN-HHHMNIVKKTEIQK GGFSK ETVE-PKK- | 1101 |
| WP_002330729 | 1049 | --T----------P VCDEN-GE-IFWDKSKSIAQVKKVIN-HHHMNIVKKTEIQK GGFSE ETVE-PKK- | 1100 |
| WP_002335161 | 1050 | --T----------P VCDEN-GE-IFWDKSKSIAQVKKVIN-HHHMNIVKKTEIQK GGFSK ETVE-PKK- | 1101 |
| WP_002345439 | 1050 | --T----------P VCDEN-GE-IFWDKSKSIAQVKKVIN-HHHMNIVKKTEIQK GGFSK ETVE-PKK- | 1101 |
| WP_034867970 | 1040 | --E----------P FCDEN-GE-IYWEKSHHLPRIKKVLS-SHQVNVVKKVEQQK GGFYK ETVN-SKE- | 1091 |
| WP_047937432 | 1050 | --T----------P VCDEN-GE-IFWDKSKSIAQVKKVIN-HHHMNIVKKTEIQK GGFSK ETVE-PKK- | 1101 |
| WP_010720994 | 1040 | --E----------P FCDEN-GE-IYWEKSHHLPRIKKVLS-SHQVNVVKKVEQQK GGFYK ETVN-SKE- | 1091 |
| WP_010737004 | 1040 | --E----------P FCDEN-GE-IYWEKSHHLPRIKKVLS-SHQVNVVKKVEQQK GGFYK ETVN-SKE- | 1091 |
| WP_034700478 | 1040 | --E----------P FCDEN-GE-IYWEKSHHLPRIKKVLS-SHQVNVVKKVEQQK GGFYK ETVN-SKE- | 1091 |
| WP_007209003 | 1038 | --D----------- IINDD-GE-ILWNKQETIAQVIKTLG-MHQVNVVKKVEIQK GGFSK ESIQ-PKG- | 1089 |
| WP_023519017 | 1034 | --E----------I ICDEQ-GE-VIWNKKRDLSTIKKTIG-AHQVNIVKKVEKQK GGFYK ETIN-SKA- | 1085 |
| WP_010770040 | 1046 | --A----------V IIDEN-GE-ILWDK-KNIATVKKVMS-YPQMNIVKKPEIQT GSFSK ETIK-PKG- | 1096 |
| WP_048604708 | 1043 | --K----------V IIDEN-GE-ILWNQ-KKIVTVKKVMN-YRQMNIVKKVEIQK GGFSK ESIL-PKG- | 1093 |
| WP_010750235 | 1043 | --E----------Q FCDEN-GE-IFWDKRKHIQQIKKVIS-SHQVNIVKKVEVQT GSFYK ETVN-TKE- | 1094 |
| AII16583 | 1091 | LAN-GEIRKRPLIE TNGET-GE-IVWDKGRDFATVRKVLS-MPQVNIVKKTEVQT GGFSK ESIL-PKR- | 1153 |
| WP_029073316 | 1053 | --D----------- ----T-GE-VVWDP-EWISRIKKCFY-YKDCFVTKKLEENN GSFFN -TVR-PNDe | 1099 |
| WP_031589969 | 1053 | --D----------- ----T-GE-IVWDP-NYIDRIKKCFY-YKDCFVTKKLEENN GTFFN -TVL-PNDt | 1099 |
| KDA45870 | 1035 | YPF----------- -----------WDKARDLPTIKRYLY-RAQVNKVRKAERQT GGFSD EMLV-PKS- | 1078 |
| WP_039099354 | 1044 | -------------E LVDEN-TEaVIWNKESGLAYLNKIYQ-FKKILVTREVHENS GALEN QTLYaAKDd | 1097 |
| AKP02966 | 1063 | --N-------GTTQ --DRNtGE-IIWNVG-FRDKILKIFN-YHQCNVTRKTEIKT GQFYD QTIYSPKNp | 1118 |
| WP_010991369 | 1045 | --D----------R IIDEN-GE-ILWDK-KYLDTVKKVMS-YRQMNIVKKTEIQK GEFSK ATIK-PKG- | 1095 |
| WP_033838504 | 1045 | --D----------R IIDEN-GE-ILWDK-KYLDTVKKVMS-YRQMNIVKKTEIQK GEFSK ATIK-PKG- | 1095 |
| EHN60060 | 1048 | --D----------R IIDEN-GE-ILWDK-KYLDTVKKVMS-YRQMNIVKKTEIQK GEFSK ATIK-PKG- | 1098 |
| EFR89594 | 814 | --D----------R IIDEN-GE-ILWDK-KYLDTVKKVMS-YRQMNIVKKTEIQK GEFSK ATIK-PKG- | 864 |
| WP_038409211 | 1045 | --N----------Q IIDKN-GE-ILWDN-RYLDTIKKVLS-YROMNIVKKTEIQK GEFSN ATVN-PKG- | 1095 |
| EFR95520 | 664 | --N----------Q IIDKN-GE-ILWDN-RYLDTIKKVLS-YROMNIVKKTEIQK GEFSN ATVN-PKG- | 714 |
| WP_003723650 | 1045 | --E----------R IIDEN-GE-ILWDK-KYLETIKKVLD-YROMNIVKKTEIQK GEFSK ATIK-PKG- | 1095 |
| WP_003727705 | 1045 | --E----------R IIDEN-GE-ILWDK-KYLETIKKVLD-YRQINIVKKTEIQK GEFSK ATIK-PKG- | 1095 |
| WP_003730785 | 1045 | --E----------R IIDEN-GE-ILWDK-KYLETIKKVLD-YRQINIVKKTEIQK GEFSK ATIK-PKG- | 1095 |
| WP_003733029 | 1045 | --D----------R IIDEN-GE-ILWDK-RYLETVKKVLG-YRQMNIVKKTEIQK GEFSN VTPN-PKG- | 1095 |
| WP_003739838 | 1045 | --E----------R IIDEN-GE-ILWDK-KYLETIKKVLD-YRQMNIVKKTEIQK GEFSK ATIK-PKG- | 1095 |
| WP_014601172 | 1045 | --E----------R IIDEN-GE-ILWDK-KYLETIKKVLD-YROMNIVKKTEIQK GEFSK ATIK-PKG- | 1095 |
| WP_023548323 | 1045 | --E----------R IIDEN-GE-ILWDK-KYLETIKKVLN-YROMNIVKKTEIQK GEFSN QNPK-PRG- | 1095 |
| WP_031665337 | 1045 | --E----------R IIDEN-GE-ILWDK-KYLETIKKVLD-YROMNIVKKTEIQK GEFSK ATIK-PKG- | 1095 |
| WP_031669209 | 1045 | --E----------R IIDEN-GE-ILWDK-RYLETVKKVLG-YROMNIVKKTEIQK GEFSN VTPN-PKG- | 1095 |
| WP_033920898 | 1045 | --E----------R IIDEN-GE-ILWDK-KYLETIKKVLN-YRQMNIVKKTEIQK GEFSN QNPK-PRG- | 1095 |
| AKI42028 | 1048 | --E----------R IIDEN-GE-ILWDK-KYLETIKKVLD-YRQMNIVKKTEIQK GEFSK ATIK-PKG- | 1098 |
| AKI50529 | 1048 | --E----------R IIDEN-GE-ILWDK-KYLETIKKVLN-YRQMNIVKKTEIQK GEFSN QNPK-PRG- | 1098 |
| EFR83390 | 493 | --E----------R IIDEN-GE-ILWDK-KYLETIKKVLD-YRQMNIVKKTEIQK GEFSK ATIK-PKG- | 543 |
| WP_046323366 | 1045 | --D----------R IIDEN-GE-ILWDK-KYLDTIKKVLN-YRQMNIVKKTEIQK GEFSN ATAN-PKG- | 1095 |
| AKE81011 | 1068 | LAN-GEIRKRPLIE TNGET-GE-IVWDKGRDFATVRKVLS-MPQVNIVKKTEVQT GGFSK ESIL-PKR- | 1130 |
| CUO82355 | 1043 | --D----------- ------GK-LIWNP-DLINEIKKCFY-YKDCYCTTKLDQKS GQMFN -TVL-PNDa | 1088 |
| WP_033162887 | 1043 | --D----------- ----T-GE-VMWDP-AKIGKIKSCFY-YKDVYVTKKLEONS GTLEN -TVL-PNDa | 1089 |
| AGZ01981 | 1085 | LAN-GEIRKRPLIE TNGET-GE-IVWDKGRDFATVRKVLS-MPQVNIVKKTEVQT GGFSK ESIL-PKR- | 1147 |
| AKA60242 | 1052 | LAN-GEIRKRPLIE TNGET-GE-IVWDKGRDFATVRKVLS-MPQVNIVKKTEVQT GGFSK ESIL-PKR- | 1114 |
| AKS40380 | 1052 | LAN-GEIRKRPLIE TNGET-GE-IVWDKGRDFATVRKVLS-MPQVNIVKKTEVQT GGFSK ESIL-PKR- | 1114 |
| 4UN5_B | 1056 | LAN-GEIRKRPLIE TNGET-GE-IVWDKGRDFATVRKVLS-MPQVNIVKKTEVQT GGFSK ESIL-PKR- | 1118 |
| WP_010922251 | 1115 | --NSD KLIA----RKKDW-DPKKYGGFDSPTVAYSV-LVVAK--VE--KGKSKKLKSVKELLGITIME RSSFEK | 1176 |
| WP_039695303 | 1121 | --DSD KLIPRKTKKV-YW-DTKKYGGFDSPTVAYSV-FVVAD--VE--KGKAKKLKTVKELVGISIME RSFFEE | 1185 |
| WP_045635197 | 1119 | --NSD KLIPRKT-KDILL-DTTKYGGFDSPVIAYSI-LLIAD--IE--KGKAKKLKTVKTLVGITIME KAAFEE | 1183 |
| 5AXW_A | 853 | --EKN -LYKYYEeTGNYL---TKYSKKDNGPVIKKI------------KYYGNKLNAHLDITDDYPNS -VKLSL | 912 |
| WP_009880683 | 799 | --NSD KLIA----RKKDW-DPKKYGGFDSPTVAYSV-LVVAK--VE--KGKSKKLKSVKELVGITIME RSSFEK | 860 |
| WP_010922251 | 1115 | --NSD KLIA----RKKDW-DPKKYGGFDSPTVAYSV-LVVAK--VE--KGKSKKLKSVKELLGITIME RSSFEK | 1176 |
| WP_011054416 | 1115 | --NSD KLIA----RKKDW-DPKKYGGFDSPTVAYSV-LVVAK--VE--KGKSKKLKSVKELLGITIME RSSFEK | 1176 |
| WP_011284745 | 1115 | --NSD KLIA----RKKDW-DPKKYGGFDSPTVAYSV-LVVAK--VE--KGKSKKLKSVKELLGITIME RSSFEK | 1176 |
| WP_011285506 | 1115 | --NSD KLIA----RKKDW-DPKKYGGFDSPTVAYSV-LVVAK--VE--KGKSKKLKSVKELLGITIME RSSFEK | 1176 |
| WP_011527619 | 1115 | --NSD KLIA----RKKDW-DPKKYGGFDSPTVAYSV-LVVAK--VE--KGKSKKLKSVKELLGITIME RSSFEK | 1176 |
| WP_012560673 | 1115 | --NSD KLIA----RKKDW-DPKKYGGFDSPTVAYSV-LVVAK--VE--KGKSKKLKSVKELVGITIME RSSFEK | 1176 |
| WP_014407541 | 1114 | --NSD KLIA----RKKDW-DPKKYGGFDSPTVAYSV-LVVAK--VE--KGKSKKLKSVKELLGITIME RSSFEK | 1175 |
| WP_020905136 | 1115 | --NSD KLIA----RKKDW-DPKKYGGFDSPTVAYSV-LVVAK--VE--KGKSKKLKSVKELLGLTIME RSSFEK | 1176 |
| WP_023080005 | 1114 | --NSD KLIA----RKKDW-DPKKYGGFDSPTVAYSV-LVVAK--VE--KGKSKKLKSVKELLGITIME RSSFEK | 1175 |
| WP_023610282 | 1114 | --NSD KLIA----RKKDW-DPKKYGGFDSPTVAYSV-LVVAK--VE--KGKSKKLKSVKELLGITIME RSSFEK | 1175 |
| WP_030125963 | 1115 | --NSD KLIA----RKKDW-DPKKYGGFDSPTVAYSV-LVVAK--VE--KGKSKKLKSVKELLGITIME RSSFEK | 1176 |
| WP_030126706 | 1115 | --NSD KLIA----RKKDW-DPKKYGGFDSPTVAYSV-LVVAK--VE--KGKSKKLKSVKELLGITIME RSSFEK | 1176 |
| WP_031488318 | 1115 | --NSD KLIA----RKKDW-DPKKYGGFDSPTVAYSV-LVVAK--VE--KGKSKKLKSVKELLGITIME RSSFEK | 1176 |
| WP_032460140 | 1115 | --NSD KLIA----RKKDW-DPKKYGGFDSPTVAYSV-LVVAK--VE--KGKSKKLKSVKELVGITIME RSSFEK | 1176 |
| WP_032461047 | 1115 | --NSD KLIA----RKKDW-DPKKYGGFDSPTVAYSV-LVVAK--VE--KGKSKKLKSVKELVGITIME RSSFEK | 1176 |
| WP_032462016 | 1115 | --NSD KLIA----RKKDW-DPKKYGGFDSPTVAYSV-LVVAK--VE--KGKSKKLKSVKELLGITIME RSSFEK | 1176 |
| WP_032462936 | 1115 | --NSD KLIA----RKKDW-DPKKYGGFDSPTVAYSV-LVVAK--VE--KGKSKKLKSVKELLGITIME RSSFEK | 1176 |
| WP_032464890 | 1115 | --NSD KLIA----RKKDW-DPKKYGGFDSPTVAYSV-LVVAK--VE--KGKSKKLKSVKELLGITIME RSSFEK | 1176 |
| WP_033888930 | 940 | --NSD KLIA----RKKDW-DPKKYGGFDSPTVAYSV-LVVAK--VE--KGKSKKLKSVKELLGITIME RSSFEK | 1001 |
| WP_038431314 | 1115 | --NSD KLIA----RKKDW-DPKKYGGFDSPTVAYSV-LVVAK--VE--KGKSKKLKSVKELLGITIME RSSFEK | 1176 |
| WP_038432938 | 1114 | --NSD KLIA----RKKDW-DPKKYGGFDSPTVAYSV-LVVAK--VE--KGKSKKLKSVKELLGITIME RSSFEK | 1175 |
| WP_038434062 | 1115 | --NSD KLIA----RKKDW-DPKKYGGFDSPTVAYSV-LVVAK--VE--KGKSKKLKSVKELLGITIME RSSFEK | 1176 |
| BAQ51233 | 1026 | --NSD KLIA----RKKDW-DPKKYGGFDSPTVAYSV-LVVAK--VE--KGKSKKLKSVKELLGITIME RSSFEK | 1087 |
| KGE60162 | 290 | --NSD KLIA----RKKDW-DPKKYGGFDSPTVAYSV-LVVAK--VE--KGKSKKLKSVKELLGITIME RSSFEK | 351 |
| KGE60856 | 53 | --NSD KLIA----RKKDW-DPKKYGGFDSPTVAYSV-LVVAK--VE--KGKSKKLKSVKELLGITIME RSSFEK | 114 |
| WP_002989955 | 1115 | --NSD KLIA----RKKDW-DPKKYGGFDSPTVAYSV-LVVAK--VE--KGKSKKLKSVKELLGITIME RSSFEK | 1176 |
| WP_003030002 | 1094 | --ESD KLIPRKT-KNSYW-NPKKYGGFDSPVVAYSI-LVFAD--VE--KGKSKKLRKVQDMVGITIME KKRFEK | 1158 |
| WP_003065552 | 1122 | --DSD KLIPRKTKKA-YW-DTKKYGGFDSPTVAYSV-FVVAD--VE--KGKAKKLKTVKELVGISIME RSFFEE | 1186 |
| WP_001040076 | 1113 | --NSD KLIPRKT-KDIYL-DPKKYGGFDSPIVAYSV-LVVAD--IK--KGKAQKLKTVTELLGITIME RSRFEK | 1177 |
| WP_001040078 | 1121 | --NSD KLIPRKT-KDIYL-DPKKYGGFDSPIVAYSV-LVVAD--IK--KGKAQKLKTVTELLGITIME RSKFEK | 1185 |
| WP_001040080 | 1113 | --NSD KLIPRKT-KDIYL-DPKKYGGFDSPIVAYSV-LVVAD--IK--KGKAQKLKTVTELLGITIME RSRFEK | 1177 |
| WP_001040081 | 1113 | --NSD KLIPRKT-KDIYL-DPKKYGGFDSPIVAYSV-LVVAD--IK--KGKAQKLKTVTELLGITIME RSRFEK | 1177 |
| WP_001040083 | 1113 | --NSD KLIPRKT-KDIYL-DPKKYGGFDSPIVAYSV-LVVAD--IK--KGKAQKLKTVTELLGITIME RSRFEK | 1177 |
| WP_001040085 | 1113 | --NSD KLIPRKT-KDIYL-DPKKYGGFDSPIVAYSV-LVVAD--IK--KGKAQKLKTVTELLGITIME RSRFEK | 1177 |
| WP_001040087 | 1113 | --NSD KLIPRKT-KDIYL-DPKKYGGFDSPIVAYSV-LVVAD--IK--KGKAQKLKTVTELLGITIME RSRFEK | 1177 |
| WP_001040088 | 1113 | --NSD KLIPRKT-KDIYL-DPKKYGGFDSPIVAYSV-LVVAD--IK--KGKAQKLKTVTELLGITIME RSRFEK | 1177 |
| WP_001040089 | 1113 | --NSD KLIPRKT-KDIYL-DPKKYGGFDSPIVAYSV-LVVAD--IK--KGKAQKLKTVTELLGITIME RSRFEK | 1177 |
| WP_001040090 | 1113 | --NSD KLIPRKT-KDIYL-DPKKYGGFDSPIVAYSV-LVVAD--IK--KGKAQKLKTVTELLGITIME RSRFEK | 1177 |
| WP_001040091 | 1113 | --NSD KLIPRKT-KDIYL-DPKKYGGFDSPIVAYSV-LVVAD--IK--KGKAQKLKTVTELLGITIME RSRFEK | 1177 |
| WP_001040092 | 1113 | --NSD KLIPRKT-KDIYL-DPKKYGGFDSPIVAYSV-LVLAD--IK--KGKAQKLKTVKELIGITIME RERFEK | 1177 |
| WP_001040094 | 1113 | --NSD KLIPRKT-KDIYL-DPKKYGGFDSPIVAYSV-LVVAD--IK--KGKAQKLKTVTELLGITIME RSRFEK | 1177 |
| WP_001040095 | 1113 | --NSD KLIPRKT-KDIYL-DPKKYGGFDSPIVAYSV-LVVAD--IK--KGKAQKLKTVTELLGITIME RSRFEK | 1177 |
| WP_001040096 | 1113 | --NSD KLIPRKT-KDIYL-DPKKYGGFDSPIVAYSV-LVVAD--IK--KGKAQKLKTVTELLGITIME RSRFEK | 1177 |
| WP_001040097 | 1113 | --NSD KLIPRKT-KDIYL-DPKKYGGFDSPIVAYSV-LVVAD--IK--KGKAQKLKTVTELLGITIME RSRFEK | 1177 |
| WP_001040098 | 1113 | --NSD KLIPRKT-KDIYL-DPKKYGGFDSPIVAYSV-LVVAD--IK--KGKAQKLKTVTELLGITIME RSRFEK | 1177 |
| WP_001040099 | 1113 | --NSD KLIPRKT-KDIYL-DPKKYGGFDSPKVAYSV-LVVAD--IK--KGKAQKLKTVTELLGITIME RSRFEK | 1177 |
| WP_001040100 | 1113 | --NSD KLIPRKT-KDIYL-DPKKYGGFDSPIVAYSV-LVVAD--IK--KGKAQKLKTVTELLGITIME RSRFEK | 1177 |
| WP_001040104 | 1113 | --NSD KLIPRKT-KDIYL-DPKKYGGFDSPIVAYSV-LVVAD--IK--KGKAQKLKTVTELLGITIME RSRFEK | 1177 |
| WP_001040105 | 1113 | --NSD KLIPRKT-KDIYL-DPKKYGGFDSPIVAYSV-LVVAD--IK--KGKAQKLKTVTELLGITIME RSRFEK | 1177 |
| WP_001040106 | 1113 | --NSD KLIPRKT-KDIYL-DPKKYGGFDSPIVAYSV-LVVAD--IK--KGKAQKLKTVTELLGITIME RFRFEK | 1177 |
| WP_001040107 | 1113 | --NSD KLIPRKT-KDIYL-DPKKYGGFDSPIVAYSV-LVVAD--IK--KGKAQKLKTVTELLGITIME RFRFEK | 1177 |
| WP_001040108 | 1113 | --NSD KLIPRKT-KDIYL-DPKKYGGFDSPIVAYSV-LVVAD--IK--KGKAQKLKTVTELLGITIME RFRFEK | 1177 |
| WP_001040109 | 1113 | --NSD KLIPRKT-KDIYL-DPKKYGGFDSPIVAYSV-LVVAD--IK--KGKAQKLKTVTELLGITIME RFRFEK | 1177 |
| WP_001040110 | 1113 | --NSD KLIPRKT-KDIYL-DPKKYGGFDSPIVAYSV-LVVAD--IK--KGKAQKLKTVTELLGITIME RFRFEK | 1177 |
| WP_015058523 | 1113 | --NSD KLIPRKT-KDIYL-DPKKYGGFDSPIVAYSV-LVLAD--IK--KGKAQKLKTVKELIGITIME RERFEK | 1177 |
| WP_017643650 | 1113 | --NSD KLIPRKT-KDIYL-DPKKYGGFDSPIVAYSV-LVVAD--IK--KGKAQKLKTVTELLGITIME RSRFEK | 1177 |
| WP_017647151 | 1113 | --NSD KLIPRKT-KDIYL-DPKKYGGFDSPIVAYSV-LVVAD--IK--KGKAQKLKTVTELLGITIME RSRFEK | 1177 |
| WP_017648376 | 1113 | --NSD KLIPRKT-KDIYL-DPKKYGGFDSPIVAYSV-LVVAD--IK--KGKAQKLKTVTELLGITIME RSRFEK | 1177 |
| WP_017649527 | 1113 | --NSD KLIPRKT-KDIYL-DPKKYGGFDSPIVAYSV-LVVAD--IK--KGKAQKLKTVTELLGITIME RSRFEK | 1177 |
| WP_017771611 | 1113 | --NSD KLIPRKT-KDIYL-DPKKYGGFDSPIVAYSV-LVVAD--IK--KGKAQKLKTVTELLGITIME RFRFEK | 1177 |
| WP_017771984 | 1113 | --NSD KLIPRKT-KDIYL-DPKKYGGEDSPIVAYSV-LVAAD--IK--KGKAQKLKTVTELLGITIME RSRFEK | 1177 |
| CFQ25032 | 1113 | --NSD KLIPRKT-KDIYL-DPKKYGGFDSPIVAYSV-LVVAD--IK--KGKAQKLKTVTELLGITIME RSRFEK | 1177 |
| CFV16040 | 1113 | --NSD KLIPRKT-KDIYL-DPKKYGGFDSPIVAYSV-LVVAD--IK--KGKAQKLKTVTELLGITIME RSRFEK | 1177 |
| KLJ37842 | 1113 | --NSD KLIPRKT-KDIYL-DPKKYGGFDSPIVAYSV-LVVAD--IK--KGKAQKLKTVTELLGITIME RSRFEK | 1177 |
| KLJ72361 | 1113 | --NSD KLIPRKT-KDIYL-DPKKYGGFDSPIVAYSV-LVVAD--IK--KGKAQKLKTVTELLGITIME RSRFEK | 1177 |
| KLL20707 | 1127 | --NSD KLIPRKT-KDIYL-DPKKYGGFDSPIVAYSV-LVVAD--IK--KGKAQKLKTVTELLGITIME RSRFEK | 1191 |
| KLL42645 | 1113 | --NSD KLIPRKT-KDIYL-DPKKYGGFDSPIVAYSV-LVVAD--IK--KGKAQKLKTVTELLGITIME RFRFEK | 1177 |
| WP_047207273 | 1113 | --NSD KLIPRKT-KDIYL-DPKKYGGFDSPIVAYSV-LVVAD--IK--KGKAQKLKTVTELLGITIME RSRFEK | 1177 |
| WP_047209694 | 1113 | --NSD KLIPRKT-KDIYL-DPKKYGGFDSPIVAYSV-LVVAD--IK--KGKAQKLKTVTELLGITIME RSRFEK | 1177 |
| WP_050198062 | 1113 | --NSD KLIPRKT-KDIYL-DPKKYGGFDSPIVAYSV-LVVAD--IK--KGKAQKLKTVTELLGITIME RSRFEK | 1177 |
| WP_050201642 | 1113 | --NSD KLIPRKT-KDIYL-DPKKYGGFDSPIVAYSV-LVVAD--IK--KGKAQKLKTVTELLGITIME RSRFEK | 1177 |
| WP_050204027 | 1113 | --NSD KLIPRKT-KDIYL-DPKKYGGFDSPIVAYSV-LVVAD--IK--KGKAQKLKTVTELLGITIME RFRFEK | 1177 |
| WP_050881965 | 1113 | --NSD KLIPRKT-KDIYL-DPKKYGGFDSPIVAYSV-LVVAD--IK--KGKAQKLKTVTELLGITIME RSRFEK | 1177 |
| WP_050886065 | 1113 | --NSD KLIPRKT-KDIYL-DPKKYGGFDSPIVAYSV-LVVAD--IK--KGKAQKLKTVTELLGITIME RSRFEK | 1177 |
| AHN30376 | 1113 | --NSD KLIPRKT-KDIYL-DPKKYGGFDSPIVAYSV-LVLAD--IK--KGKAQKLKTVKELIGITIME RERFEK | 1177 |
| EAO78426 | 1113 | --NSD KLIPRKT-KDIYL-DPKKYGGFDSPIVAYSV-LVVAD--IK--KGKAQKLKTVTELLGITIME RSRFEK | 1177 |
| CCW42055 | 1113 | --NSD KLIPRKT-KDIYL-DPKKYGGFDSPIVAYSV-LVVAD--IK--KGKAQKLKTVTELLGITIME RSRFEK | 1177 |
| WP_003041502 | 1123 | --DSS ENLVGVK-RNL---DPKKYGGYAGISNSYAV-LVKAI--IE--KGVKKKETMVLEFQGISILD RITFEK | 1185 |
| WP_037593752 | 1095 | --ESD KLIPRKT-KNSYW-NPKKYGGFDSPVVAYSI-LVFAD--VE--KGKSKKLRKVQDMVGITIME KKRFEK | 1159 |
| WP_049516684 | 1095 | --ESD KLIPRKT-KNSYW-NPKKYGGFDSPVVAYSI-LVFAD--VE--KGKSKKLRKVQDMVGITIME KKRFEK | 1159 |
| GAD46167 | 1094 | --ESD KLIPRKT-KNSYW-NPKKYGGFDSPVVAYSI-LVFAD--VE--KGKSKKLRKVQDMVGITIME KKRFEK | 1158 |
| WP_018363470 | 1126 | --DSD KLIPRKTKKV-LW-EPKKYGGFDSPTVAYSV-LVVAD--VE--KGKTKKLKTVKELVGISIME RSFFEK | 1190 |
| WP_003043819 | 1124 | --ESA KLIP----RKKGW-DTRKYGGFGSPTVAYSI-LVVAK--VE--KGKAKKLKSVKVLVGITIME KGSYEK | 1185 |
| WP_006269658 | 1094 | --ESD KLIPRKT-KNSYW-DPKKYGGFDSPVVAYSI-LVFAD--VE--KGKSKKLRKVQDMVGITIME KKRFEK | 1158 |
| WP_048800889 | 1114 | --DSD KLIARKTKEN-YW-DTKKYGGFDSPTVAYSV-LVVAD--IK--KGKAKKLKTVKELVGISIME RPFFEK | 1178 |
| WP_012767106 | 1114 | --SFD KLIS----RKHRF-ESSKYGGFGSPTVTYSV-LVVAKskVQ--DGKVKKIKTGKELIGMTLLD KLVFEK | 1177 |
| WP_014612333 | 1114 | --SFD KLIS----RKHRF-ESSKYGGFGSPTVTYSV-LVVAKSKVQ--DGKVKKIKTGKELIGITLLD KLVFEK | 1177 |
| WP_015017095 | 1114 | --SFD KLIS----RKHRF-ESSKYGGFGSPTVTYSV-LVVAKskVQ--DGKVKKIKTGKELIGITLLD KLVFEK | 1177 |
| WP_015057649 | 1114 | --SED KLIS----RKHRF-ESSKYGGFGSPTVTYSV-LVVAKskVQ--DGKVKKIKTGKELIGITLLD KLVFEK | 1177 |
| WP_048327215 | 1114 | --SFD KLIS----RKHRF-ESSKYGGFGSPTVTYSV-LVVAKckVQ--DGKVKKIKTGKELIGITLLD KLVFEK | 1177 |
| WP_049519324 | 1114 | --SFD KLIS----RKHRF-ESSKYGGFGSPTVTYSV-LVVAKskVQ--DGKVKKIKTGKELIGITLLD KLVFEK | 1177 |
| WP_012515931 | 1095 | --NSD KLIP----RKNNW-DTRKYGGFDSPTVAYSV-LVIAK--ME--KGKAKVLKPVKEMVGITIME RTAFEE | 1156 |
| WP_021320964 | 1095 | --NSD KLIP----RKNNW-DTRKYGGFDSPTVAYSV-LVIAK--ME--KGKAKVLKPVKEMVGITIME RIAFEE | 1156 |
| WP_037581760 | 1095 | --NSD KLIP----RKNNW-DTRKYGGFDSPTVAYSV-LVIAK--ME--KGKAKVLKPVKEMVGITIME RIAFEE | 1156 |
| WP_004232481 | 1124 | --DSD KLIPRKTKKL-QW-ETQKYGGFDSPTVAYSV-LVVAD--VE--KGKTRKLKTVKELVGISIME RSSFEE | 1188 |
| WP_009854540 | 1119 | --DSD KLIPRKTKKV-YW-DTKKYGGFDSPTVAYSV-FVVAD--VE--KGKAKKLKTVKELVGISIME RSFFEE | 1183 |
| WP_012962174 | 1120 | --NSD KLIPRKTKKF-RW-DTPKYGGFDSPNIAYSV-FVIAD--VE--KGKAKKLKTVKELVGISIME RSSFEE | 1184 |
| WP_039695303 | 1121 | --DSD KLIPRKTKKV-YW-DTKKYGGFDSPTVAYSV-FVVAD--VE--KGKAKKLKTVKELVGISIME RSFFEE | 1185 |
| WP_014334983 | 1124 | --DSD KLIPRKTKKV-YW-NTKKYGGFDSPTVAYSV-LVVAD--IE--KGKAKKLKTVKELVGISIME RSFFEE | 1188 |
| WP_003099269 | 1115 | --DSD KLIA----RKKSW-DPKKYGGFDSPIIAYSV-LVVAK--IA--KGKTQKLKTIKELVGIKIME QDEFEK | 1176 |
| AHY15608 | 1115 | --DSD KLIA----RKKSW-DPKKYGGFDSPIIAYSV-LVVAK--IA--KGKTQKLKTIKELVGIKIME QDEFEK | 1176 |
| AHY17476 | 1115 | --DSD KLIA----RKKSW-DPKKYGGFDSPIIAYSV-LVVAK--IA--KGKTQKLKTIKELVGIKIME QDEFEK | 1176 |
| ESR09100 | 1 | ----- -------------------------------------------------------------ME QDEFEK | 8 |
| AGM98575 | 1115 | --DSD KLIA----RKKSW-DPKKYGGFDSPIIAYSV-LVVAK--IA--KGKTQKLKTIKELVGIKIME QDEFEK | 1176 |
| ALF27331 | 1094 | --NSD KLIPRKT-KKFYW-DTKKYGGFDSPIVAYSI-LVIAN--IE--KGKSKKLKLVKDLVGITIME RTIFEK | 1158 |
| WP_018372492 | 1109 | ---NN KLIP----RKKDW-SVDKYGGFIEPAESYSLaIFYTD--IN-----GKKPKKKSTIIAISRME KKDYEK | 1167 |
| WP_045618028 | 1125 | VVDAS KLTPIKS-G---L-SPEKYGGYARPTIAYSV-LVIAD--IE--KGKAKKLKRIKEMVGITVQD KKKFEA | 1188 |
| WP_045635197 | 1119 | --NSD KLIPRKT-KDILL-DTTKYGGFDSPVIAYSI-LLIAD--IE--KGKAKKLKTVKTLVGITIME KAAFEE | 1183 |
| WP_002263549 | 1094 | --NSD KLIPRKT-KKFYW-DTKKYGGFDSPIVAYSI-LVIAD--IE--KGKSKKLKTVKALVGVTIME KMTFER | 1158 |
| WP_002263887 | 1094 | --NSD KLIPRKT-KKFYW-DTKKYGGFDSPIVAYSI-LVIAD--IE--KGKSKKLKTVKALVGVTIME KMTFER | 1158 |
| WP_002264920 | 1094 | --DSD KLIPRKT-KKFYW-DTKKYGGFDSPIVAYSI-LVIAD--IE--KGKSKKLKTVKALVGVTIME KMTFER | 1158 |
| WP_002269043 | 1094 | --NSD KLIPRKT-KKFYW-DTKKYGGFDSPIVAYSI-LVIAD--IE--KGKSKKLKTVKALVGVTIME KMTFER | 1158 |
| WP_002269448 | 1094 | --NSD KLIPRKT-KKFYW-DTKKYGGFDSPIVAYSI-LVIAD--IE--KGKSKKLKTVKALVGVTIME KMTFER | 1158 |
| WP_002271977 | 1094 | --NSD KLIPRKT-KKFYW-DTKKYGGFDSPIVAYSI-LVIAD--IE--KGKSKKLKTVKALVGVTIME KMTFER | 1158 |
| WP_002272766 | 1094 | --NSD KLIPRKT-KKFYW-DTKKYGGFDSPIVAYSI-LVIAD--IE--KGKSKKLKTVKALVGVTIME KMTFER | 1158 |
| WP_002273241 | 1094 | --NSD KLIPRKT-KKFYW-DTKKYGGFDSPIVAYSI-LVIAD--IE--KGKSKKLKTVKALVGVTIME KMTFER | 1158 |
| WP_002275430 | 1094 | --NSD KLIPRKT-KKFYW-DTKKYGGFDSPIVAYSI-LVIAD--IE--KGKSKKLKTVKALVGVTIME KMTFER | 1158 |
| WP_002276448 | 1094 | --NSD KLIPRKT-KKFYW-DTKKYGGFDSPIVAYSI-LVIAD--IE--KGKSKKLKTVKALVGVTIME KMTFER | 1158 |
| WP_002277050 | 1112 | --PLE KLVPLKK----AL-NPEKYGGYOKPTTAYPI-LLIVD---------------TKQLIPISVMD KKRFEQ | 1166 |
| WP_002277364 | 1094 | --NSD KLIPRKT-KKFYW-DTKKYGGFDSPIVAYSI-LVIAD--IE--KGKSKKLKTVKALVGVTIME KMTFER | 1158 |
| WP_002279025 | 1094 | --NSD KLIPRKT-KKFYW-DTKKYGGFDSPIVAYSI-LVIAD--IE--KGKSKKLKTVKALVGVTIME KMTFER | 1158 |
| WP_002279859 | 1094 | --DSD KLIPRKT-KKFYW-DTKKYGGFDSPIVAYSI-LVIAD--IE--KGKSKKLKTVKALVGVTIME KMTFER | 1158 |
| WP_002280230 | 1094 | --NSD KLIPRKT-KKFYW-DTKKYGGFDSPIVAYSI-LVIAD--IE--KGKSKKLKTVKALVGVTIME KMTFER | 1158 |
| WP_002281696 | 1094 | --NSD KLIPRKT-KKFYW-DTKKYGGFDSPIVAYSI-LVIAD--IE--KGKSKKLKTVKALVGVTIME KMTFER | 1158 |
| WP_002282247 | 1112 | --PLE KLVPLKK----AL-NPEKYGGYQKPTTAYPI-LLIVD---------------TKQLIPISVMD KKRFEQ | 1166 |
| WP_002282906 | 1094 | --NSD KLIPRKT-KKFYW-DTKKYGGFDSPIVAYSI-LVIAD--IE--KGKSKKLKTVKALVGVTIME KMTFER | 1158 |
| WP_002283846 | 1094 | --NSD KLIPRKT-KKFYW-DTKKYGGFDSPIVAYSI-LVIAD--IE--KGKSKKLKTVKALVGVTIME KMTFER | 1158 |
| WP_002287255 | 1094 | --NSD KLIPRKT-KKFYW-DTKKYGGFDSPIVAYSI-LVIAD--IE--KGKSKKLKTVKALVGVTIME KMTFER | 1158 |
| WP_002288990 | 1094 | --NSY KLIPRKT-KKFYW-DTKKYGGFDSPIVAYSI-LVIAD--IE--KGKSKKLKTVKALVGVTIME KMTFER | 1158 |
| WP_002289641 | 1094 | --NSD KLIPRKT-KKFYW-DTKKYGGFDSPIVAYSI-LVIAD--IE--KGKSKKLKTVKALVGVTIME KMTFER | 1158 |
| WP_002290427 | 1094 | --DSD KLIPRKT-KKFYW-DTKKYGGFDSPIVAYSI-LVIAD--IE--KGKSKKLKTVKALVGVTIME KMTFER | 1158 |
| WP_002295753 | 1094 | --NSD KLIPRKT-KKFYW-DTKKYGGFDSPIVAYSI-LVIAD--IE--KGKSKKLKTVKALVGVTIME KMTFER | 1158 |
| WP_002296423 | 1094 | --NSD KLIPRKT-KKFYW-DTKKYGGFDSPIVAYSI-LVIAD--IE--KGKSKKLKTVKALVGVTIME KMTFER | 1158 |
| WP_002304487 | 1108 | --NSD KLIPRKT-KKFYW-DTKKYGGFDSPIVAYSI-LVIAD--IE--KGKSKKLKTVKALVGVTIME KMTFER | 1172 |
| WP_002305844 | 1094 | --NSD KLIPRKT-KKFYW-DTKKYGGFDSPIVAYSI-LVIAD--IE--KGKSKKLKTVKALVGVTIME KMTFER | 1158 |
| WP_002307203 | 1094 | --NSD KLIPRKT-KKFYW-DTKKYGGFDSPIVAYSI-LVIAD--IE--KGKSKKLKTVKALVGVTIME KMTFER | 1158 |
| WP_002310390 | 1094 | --DSD KLIPRKT-KKFYW-DTKKYGGFDSPIVAYSI-LVIAD--IE--KGKSKKLKTVKALVGVTIME KMTFER | 1158 |
| WP_002352408 | 1094 | --DSD KLIPRKT-KKFYW-DTKKYGGFDSPIVAYSI-LVIAD--IE--KGKSKKLKTVKALVGVTIME KMTFER | 1158 |
| WP_012997688 | 1094 | --NSD KLIPRKT-KKFYW-DTKKYGGFDSPIVAYSI-LVIAD--IE--KGKSKKLKTVKALVGVTIME KMTFER | 1158 |
| WP_014677909 | 1094 | --NSD KLIPRKT-KKFYW-DTKKYGGFDSPIVAYSI-LVIAD--IE--KGKSKKLKTVKALVGVTIME KMTFER | 1158 |
| WP_019312892 | 1094 | --NSD KLIPRKT-KKFYW-DTKKYGGFDSPIVAYSI-LVIAD--IE--KGKSKKLKTVKALVGVTIME KMTFER | 1158 |
| WP_019313659 | 1094 | --NSD KLIPRKT-KKFYW-DTKKYGGFDSPIVAYSI-LVIAD--IE--KGKSKKLKTVKALVGVTIME KMTFER | 1158 |
| WP_019314093 | 1094 | --NSD KLIPRKT-KKFYW-DTKKYGGFDSPIVAYSI-LVIAD--IE--KGKSKKLKTVKALVGVTIME KMTFER | 1158 |
| WP_019315370 | 1094 | --NSD KLIPRKT-KKFYW-DTKKYGGFDSPIVAYSI-LVIAD--IE--KGKSKKLKTVKALVGVTIME KMTFER | 1158 |
| WP_019803776 | 1094 | --NSD KLIPRKT-KKFYW-DTKKYGGFDSPIVAYSI-LVIAD--IE--KGKSKKLKTVKALVGVTIME KMTFER | 1158 |
| WP_019805234 | 1094 | --DSD KLIPRKT-KKFYW-DTKKYGGFDSPIVAYSI-LVIAD--IE--KGKSKKLKTVKALVGVTIME KMTFER | 1158 |
| WP_024783594 | 1094 | --NSD KLIPRKT-KKFYW-DTKKYGGFDSPIVAYSI-LVIAD--IE--KSKSKKLKTVKALVGVTIME KMTFER | 1158 |
| WP_024784288 | 1112 | --PLE KLVPLKK----AL-NPEKYGGYQKPTTAYPI-LLIVD---------------TKQLIPISVMD KKRFEQ | 1166 |
| WP_024784666 | 1094 | --NSD KLIPRKT-KKFYW-DTKKYGGFDSPIVAYSI-LVIAD--IE--KGKSKKLKTVKALVGVTIME KMTFER | 1158 |
| WP_024784894 | 1094 | --NSD KLIPRKT-KKFYW-DTKKYGGFDSPIVAYSI-LVIAD--IE--KGKSKKLKTVKALVGVTIME KMTFER | 1158 |
| WP_024786433 | 1112 | --PLE KLVPLKK----AL-NPEKYGGYQKPTTAYPI-LLIVD---------------TKQLIPISVMD KKRFEQ | 1166 |
| WP_049473442 | 1094 | --NSD KLIPRKT-KKFYW-DTKKYGGFDSPIVAYSI-LVIAD--IE--KGKSKKLKTVKALVGVTIME KMTFER | 1158 |
| WP_049474547 | 1094 | --NSD KLIPRKT-KKFYW-DTKKYGGEDSPIVAYSI-LVIAD--IE--KGKSKKLKTVKALVGVTIME KMTFER | 1158 |
| EMC03581 | 1087 | --NSD KLIPRKT-KKFYW-DTKKYGGFDSPIVAYSI-LVIAD--IE--KGKSKKLKTVKALVGVTIME KMTFER | 1151 |
| WP_000428612 | 1122 | --NSD KLIPRKT-KDILW-DTTKYGGFDSPVIAYSI-LLIAD--IE--KGKAKRLKTVKTLVGITIME KATFEK | 1186 |
| WP_000428613 | 1120 | --NSD KLIPRKT-KDILW-ETTKYGGFDSPVIAYSI-LLIAD--IE--KGKAKKLKTVKTLVGITIME KAAFEE | 1184 |
| WP_049523028 | 1115 | --NSD KLIPRKT-KNVQL-DTTKYGGFDSPVIAYSI-LLVAD--VE--KGKSKKLKTVKSLIGITIME KVKFEA | 1179 |
| WP_003107102 | 1084 | --DSD KLIP----RKNNW-DPKKYGGFGSPIIAYSV-LVVAK--VT--KGKSQKTKSVKELVGITIME QNEFEK | 1145 |
| WP_054279288 | 1116 | --KSS KLIP----RKNKWrDTTKYGGFNTPTVAYSV-LVVAK--VE--KGKAKKLKPVKELVGITIME RTKFEA | 1178 |
| WP_049531101 | 1125 | vvDAS KLIPIKS-G---L-SPEKYGGYARPTIAYSV-LVIAD--IE--KGKAKKLKRIKEMVGITIQD KKKFEA | 1188 |
| WP_049538452 | 1125 | vvDAS KLIPIKS-G---L-SPEKYGGYARPTIAYSV-LVIAD--IE--KGKTKKLKRIKEMIGITVQD KKIFES | 1188 |
| WP_049549711 | 1127 | vvDAS KLIPIKS-G---L-SPEKYGGYARPTIAYSV-LVIAD--IE--KGKTKKLKRIKEMVGITIQD KKKFEA | 1190 |
| WP_007896501 | 1121 | --NSD KLIE----RKKGW-DPKKYGGFDSPNTAYSI-FVVAK--VA--KRKAQKLKTVKEIVGITIME QAEYEK | 1182 |
| EFR44625 | 1073 | --NSD KLIE----RKKGW-DPKKYGGFDSPNTAYSI-FVVAK--VA--KRKAQKLKTVKEIVGITIME QAEYEK | 1134 |
| WP_002897477 | 1119 | --NSD KLIPRKT-KDILW-DTTKYGGFDSPVIAYSI-LLIAD--IE--KGKAKKLKTVKTLVGITIME KAAFEE | 1183 |
| WP_002906454 | 1124 | vvDAS KLIPIKS-S---L-SPEKYGGYARPTIAYSV-LVIAD--IEkgKGKAKKLKRIKEIVGITIQD KKKFES | 1189 |
| WP_009729476 | 1120 | --NSD KLIPRKT-KDILW-DTTKYGGFDSPVIAYSI-LLIAD--IE--KGKAKKLKTVKTLVGITIME KDAFEK | 1184 |
| COR24647 | 1110 | --GSD KLIARKT-KNNYL-STQKYGGFDSPTVAYSI-MFVAD--IE--KGKSKRLKTVKEMIGITIME RSRFES | 1174 |
| WP_000066813 | 1124 | --NSD KLIPRKT-KEILW-DTTKYGGFDSPVIAYSI-LLIAD--IE--KGKAKKLKTVKTLVGITIME KATFEK | 1188 |
| WP_009754323 | 1120 | --NSD KLIPRKT-KDILW-DTTKYGGFDSPVIAYSI-LLIAD--IE--KGKAKKLKTVKTLVGITIME KAAFEK | 1184 |
| WP_044674937 | 1113 | --DSD KLIPRKT-EKFYL-DTKKYGGFDSPTIAYSV-LLIAD--IE--KGKAKKLKRVKELIGITIME RMAFEK | 1177 |
| WP_044676715 | 1115 | --DSD KLIPRKT-EKFYL-DTKKYGGFDSPTIAYSV-LLIAD--IE--KGKAKKLKRVKELIGITIME RMAFEK | 1179 |
| WP_044680361 | 1115 | --DSD KLIPRKT-EKFYL-DTKKYGGFDSPTIAYSV-LLIAD--IE--KGKAKKLKRVKELIGITIME RMAFEK | 1179 |
| WP_044681799 | 1113 | --DSD KLIPRKT-EKFYL-DTKKYGGFDSPTIAYSV-LLIAD--IE--KGKAKKLKRVKELIGITIME RMAFEK | 1177 |
| WP_049533112 | 1123 | --DSS ENLVGVK-RNL---DPKKYGGYAGISNSYAV-LVKAI--IE--KGVKKKETMVLEFQGISILD RITFEK | 1185 |
| WP_029090905 | 1062 | --SSS KTIP----LKKHL-DTAIYGGYTAVNYASYA---LIQ--FK----KGRKLK--REIIGIPLAV QTRIDN | 1117 |
| WP_006506696 | 1085 | haDKG AVVP---vNKNRS-DVHKYGGFSG--LQYTI----VA--IEgqKKKGKKTELVKKISGVPLHL KAASIN | 1149 |
| AIT42264 | 1115 | --NSD KLIA----RKKDW-DPKKYGGFDSPTVAYSV-LVVAK--VE--KGKSKKLKSVKELLGITIME RSSFEK | 1176 |
| WP_034440723 | 1093 | k-KTE KRIP----IKNNL-DPNIYGGYIEEKMAYYI----AInyLE--NGKTKK-----AIVGISIKD KKDFEG | 1149 |
| AKQ21048 | 1115 | --NSD KLIA----RKKDW-DPKKYGGFDSPTVAYSV-LVVAK--VE--KGKSKKLKSVKELLGITIME RSSFEK | 1176 |
| WP_004636532 | 1093 | --DSS KLIP----VKEGM-DPQKYGGLSQVSEAFAV-VIT----HE--KGKKKQLK--SDLISIPIVD QKAYEQ | 1150 |
| WP_002364836 | 1099 | --PSN KLIP----VKNGL-DPQKYGGFDSPIVAYTV-LF--T--HE--KGK-KPL-IKQEILGITIME KTRFEQ | 1156 |
| WP_016631044 | 1050 | --PSN KLIP----VKNGL-DPQKYGGFDSPVVAYTV-LF--T--HE--KGK-KPL-IKQEILGITIME KTRFEQ | 1107 |
| EMS75795 | 835 | --KPD KLIQ----RKAGW-DVSKYGGFGSPVVAYAV-AFI----YE--KGKAR--KKAKAIEGITIMK QSLFEQ | 892 |
| WP_002373311 | 1099 | --PSN KLIP----VKNGL-DPQKYGGFDSPVVAYTV-LF--T--HE--KGK-KPL-IKQEILGITIME KTRFEQ | 1156 |
| WP_002378009 | 1099 | --PSN KLIP----VKNGL-DPQKYGGFDSPIVAYTV-LF--T--HE--KGK-KPL-IKQEILGITIME KTRFEQ | 1156 |
| WP_002407324 | 1099 | --PSN KLIP----VKNGL-DPQKYGGFDSPIVAYTV-LF--T--HE--KGK-KPL-IKQEILGITIME KTRFEQ | 1156 |
| WP_002413717 | 1099 | --PSN KLIP----VKNGL-DPQKYGGFDSPVVAYTV-LF--T--HE--KGK-KPL-IKQEILGITIME KTRFEQ | 1156 |
| WP_010775580 | 1101 | --PSN KLIP----VKNGL-DPQKYGGFDSPVVAYTV-LF--T--HE--KGK-KPL-IKQEILGITIME KTRFEQ | 1158 |
| WP_010818269 | 1099 | --PSN KLIP----VKNGL-DPQKYGGFDSPIVAYTV-LF--T--HE--KGK-KPL-IKQEILGITIME KTRFEQ | 1156 |
| WP_010824395 | 1099 | --PSN KLIP----VKNGL-DPQKYGGFDSPVVAYTV-LF--T--HE--KGK-KPL-IKQEILGITIME KTRFEQ | 1156 |
| WP_016622645 | 1099 | --PSN KLIP----VKNGL-DPQKYGGFDSPIVAYTV-LF--T--HE--KGK-KPL-IKQEILGITIME KTRFEQ | 1156 |
| WP_033624816 | 1099 | --PSN KLIP----VKNGL-DPQKYGGFDSPVVAYTV-LF--T--HE--KGK-KPL-IKQEILGITIME KTRFEQ | 1156 |
| WP_033625576 | 1099 | --PSN KLIP----VKNGL-DPQKYGGFDSPVVAYTV-LF--T--HE--KGK-KPL-IKQEILGITIME KTKFEQ | 1156 |
| WP_033789179 | 1099 | --PSN KLIP----VKNGL-DPQKYGGFDSPVVAYTV-LF--T--HE--KGK-KPL-IKQEILGITIME KTRFEQ | 1156 |
| WP_002310644 | 1101 | --DSS KLLP----RKNNW-DPAKYGGLGSPNVAYTV-AFT----YE--KGKAR--KRTNALEGITIME REAFEQ | 1158 |
| WP_002312694 | 1102 | --DSS KLLP----RKNNW-DPAKYGGLGSPNVAYTV-AFT----YE--KGKAR--KRTNALEGITIME REAFEQ | 1159 |
| WP_002314015 | 1102 | --DSS KLLP----RKNNW-DPAKYGGLGSPNVAYTV-AFT----YE--KGKAR--KRTNALEGITIME REAFEQ | 1159 |
| WP_002320716 | 1102 | --DSS KLLP----RKNNW-DPAKYGGLGSPNVAYTV-AFT----YE--KGKAR--KRTNALEGITIME REAFEQ | 1159 |
| WP_002330729 | 1101 | --DSS KLLP----RKNNW-DPAKYGGLGSPNVAYTV-AFT----YE--KGKAR--KRTNALEGITIME REAFEQ | 1158 |
| WP_002335161 | 1102 | --DSS KLLP----RKNNW-DPAKYGGLGSPNVAYTV-AFT----YE--KGKAR--KRTNALEGITIME REAFEQ | 1159 |
| WP_002345439 | 1102 | --DSS KLLP----RKNNW-DPTKYGGLGSPNVAYTV-AFT----YE--KGKAR--KRTNALEGITIME REAFEQ | 1159 |
| WP_034867970 | 1092 | --KPD KLIE----RKNNW-DVTKYGGFGSPVIAYAI-AFV----YA--KGKTQ--KKTRAIEGITIME QAAFEK | 1149 |
| WP_047937432 | 1102 | --DSS KLLP----RKNNW-DPAKYGGLGSPNVAYTV-AFT----YE--KGKAR--KRTNALEGITIME REAFEQ | 1159 |
| WP_010720994 | 1092 | --KPD KLIE----RKNNW-DVTKYGGFGSPVIAYAI-AFV----YA--KGKTQ--KKTKAIEGITIME QAAFEK | 1149 |
| WP_010737004 | 1092 | --KPD KLIE----RKNNW-DVTKYGGFGSPVIAYAI-AFV----YA--KGKTQ--KKTRAIEGITIME QAAFEK | 1149 |
| WP_034700478 | 1092 | --KPD KLIE----RKNNW-DVTKYGGFGSPVIAYAI-AFV----YA--KGKTQ--KKTRAIEGITIME QAAFEK | 1149 |
| WP_007209003 | 1090 | --ESQ KLIR----RKQQW-NTKKYGGFDSPVVAYAI---LLS--FD--KGK-RKARSFK-IVGITIQD RESFEG | 1147 |
| WP_023519017 | 1086 | --NPE KLIP----RKASL-DPLKYGGYGSPLVAYTV-IFI----FE--KGKQK--KVTKGIEGITVME QLRFEQ | 1143 |
| WP_010770040 | 1097 | --DSD KLIS----RKTNW-SPKLYGGFDSPQVAYSV-II--T--YE--KGK-KKVRA-KAIVGITIME QSLFKK | 1154 |
| WP_048604708 | 1094 | --DSD KLIS----RKKEW-DTTKYGGFDSPNVAYSV-VI--R--YE--KGK-TRKLV-KTIVGITIME RAAFEK | 1151 |
| WP_010750235 | 1095 | --KPD KLIK----RKNNW-DVTKYGGFGSPVVAYAV-VFT----YE--KGKNH--KKAKAIEGITIME QALFEK | 1152 |
| AII16583 | 1154 | --NSD KLIA----RKKDW-DPKKYGGFDSPTVAYSV-LVVAK--VE--KGKSKKLKSVKELLGITIME RSSFEK | 1215 |
| WP_029073316 | 1100 | hsEKG AKVP---vNKLRS-NVHKYGGFEG--LKYSI----VA--IKgkKKKGKKIIDVNKLVGIPLMY KNVDDE | 1164 |
| WP_031589969 | 1100 | nsDKD ATVP---vNKYRS-NVNKYGGFSG--VNSFI----VA--IKgkKKKGKKVIEVNKLTGIPLMY KNADEE | 1164 |
| KDA45870 | 1079 | --DSG KLLP----RKEGL-DPVKYGGYAKAVESYAV-LITAD-eVK--KGKTKKVKT---LVNIPIID SKKYEA | 1138 |
| WP_039099354 | 1098 | k-ASG QLIPAKQdRPTAL-----YGGYSGKTVAYMC---IVR--IKnkKGDLYKVCGVETSWLAQLKQ KKAFLK | 1170 |
| AKP02966 | 1119 | k---- KLIA----QKKDM-DPNIYGGFSGDNKSSIT---IVK--ID-----NNKIKPVA--IPIRLIN ----DK | 1172 |
| WP_010991369 | 1096 | --NSS KLIP----RKTNW-DPMKYGGLDSPNMAYAV-VI--E--YA--KGK-NKLVFEKKIIRVTIME RKAFEK | 1154 |
| WP_033838504 | 1096 | --NSS KLIS----RKTNW-DPMKYGGLDSPNMAYAV-VI--E--YA--KGK-NKLVFEKKIIRVTIME RKAFEK | 1154 |
| EHN60060 | 1099 | --NSS KLIS----RKTNW-DPMKYGGLDSPNMAYAV-VI--E--YA--KGK-NKLVFEKKIIRVTIME RKAFEK | 1157 |
| EFR89594 | 865 | --NSS KLIP----RKTNW-DPMKYGGLDSPNMAYAV-VI--E--YA--KGK-NKLVFEKKIIRVTIME RKAFEK | 923 |
| WP_038409211 | 1096 | --NSS KLIS----RKADW-NPIKYGGFDGSNMAYSI-VI--E--YE--KRK-KKTVIKKELIQINIME RVAFEK | 1154 |
| EFR95520 | 715 | --NSS KLIS----RKADW-NPIKYGGFDGSNMAYSI-VI--E--YE--KRK-KKTVIKKELIQINIME RVAFEK | 773 |
| WP_003723650 | 1096 | --NSS KLIP----RKENW-DPMKYGGLDSPNMAYAV-II--E--HA--KGK-KKIVIEKKLIQINIME RKMFEK | 1154 |
| WP_003727705 | 1096 | --NSS KLIP----RKENW-DPMKYGGLDSPNMAYAV-II--E--HA--KGK-KKIVIEKKLIQINIME RKMFEK | 1154 |
| WP_003730785 | 1096 | --NSS KLIP----RKENW-DPVKYGGLDSPNMAYAV-II--E--HA--KGK-KKIVIEKKLIQINIME RKMFEK | 1154 |
| WP_003733029 | 1096 | --KSN KLIP----RKKDW-DPIKYGGFDGSKMAYAI-II--E--YE--KQK-RKVRIEKKLIQINIME REAFEK | 1154 |
| WP_003739838 | 1096 | --NSS KLIP----RKENW-DPMKYGGLDSPNMAYAV-II--E--HA--KGK-KKVVFEKKIIRITIME RKAFEK | 1154 |
| WP_014601172 | 1096 | --NSS KLIP----RKENW-DPMKYGGLDSPNMAYAV-II--E--HA--KGK-KKLIFEKKIIRITIME RKMFEK | 1154 |
| WP_023548323 | 1096 | --DSS KLIP----KKTNL-NPIKYGGFEGSNMAYAI-II--E--HE--KRK-KKVTIEKKLIQINIME RKAFEK | 1154 |
| WP_031665337 | 1096 | --NSS KLIP----RKENW-DPMKYGGLDSPNMAYAV-II--E--HA--KGK-KRIVIEKKLIQINIME RKMFEK | 1154 |
| WP_031669209 | 1096 | --KSN KLIP----RKKDW-DPIKYGGFDGSKMAYAI-II--E--YE--KQK-RKVRIEKKLIQINIME REAFEK | 1154 |
| WP_033920898 | 1096 | --DSS KLIP----KKTNL-NPIKYGGFEGSNMAYAI-II--E--HE--KRK-KKVTIEKKLIQINIME RKAFEK | 1154 |
| AKI42028 | 1099 | --NSS KLIP----RKENW-DPMKYGGLDSPNMAYAV-II--E--HA--KGK-KKLIFEKKIIRITIME RKMFEK | 1157 |
| AKI50529 | 1099 | --DSS KLIP----KKTNL-NPIKYGGFEGSNMAYAI-II--E--HE--KRK-KKVTIEKKLIQINIME RKAFEK | 1157 |
| EFR83390 | 544 | --NSS KLIP----RKENW-DPMKYGGLDSPNMAYAV-II--E--HA--KGK-KKIVIEKKLIQINIME RKMFEK | 602 |
| WP_046323366 | 1096 | --NSS KLIP----RKADW-DPIKYGGFDGSNMAYAI-VI--E--HE--KRK-KKTVIKKELIQINIME RTAFEK | 1154 |
| AKE81011 | 1131 | --NSD KLIA----RKKDW-DPKKYGGFDSPTVAYSV-LVVAK--VE--KGKSKKLKSVKELLGITIME RSSFEK | 1192 |
| CUO82355 | 1089 | hsAKG AVIP---vNKNRK-DVNKYGGFSG--LQYVI----AA--IEgtKKKGKKLVKVRKLSGIPLYL KQADIK | 1153 |
| WP_033162887 | 1090 | hsEKG ATVP---1NKYRA-DVHKYGGFGN--VOSII----VA--IEgkKKKGKKLIDVRKLTSIPLHL KNAPVE | 1154 |
| AGZ01981 | 1148 | --NSD KLIA----RKKDW-DPKKYGGFDSPTVAYSV-LVVAK--VE--KGKSKKLKSVKELLGITIME RSSFEK | 1209 |
| AKA60242 | 1115 | --NSD KLIA----RKKDW-DPKKYGGFDSPTVAYSV-LVVAK--VE--KGKSKKLKSVKELLGITIME RSSFEK | 1176 |
| AKS40380 | 1115 | --NSD KLIA----RKKDW-DPKKYGGFDSPTVAYSV-LVVAK--VE--KGKSKKLKSVKELLGITIME RSSFEK | 1176 |
| 4UN5_B | 1119 | --NSD KLIA----RKKDW-DPKKYGGFDSPTVAYSV-LVVAK--VE--KGKSKKLKSVKELLGITIME RSSFEK | 1180 |
| WP_010922251 | 1177 | 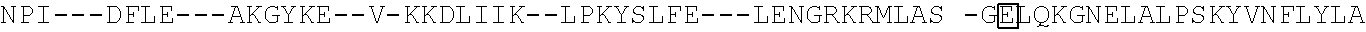 | 1239 |
| WP_039695303 | 1186 | NPV---EFLE---NKGYHN--I-REDKLIK--LPKYSLFE---FEGGRRRLLAS ASELQKGNEMVLPGYLVELLYHA | 1248 |
| WP_045635197 | 1184 | NPI---TFLE---NKGYHN--V-RKENILC--LPKYSLFE---LENGRRRLLAS AKELQKGNEIVLPVYLTTLLYHS | 1246 |
| 5AXW_A | 913 | KPYrfdVYLD---NGVYKFvtV-KNLDVIK----KENYYE---VNSKAYEEAKK -KKISNQAEFIASFYNNDLIKIN | 978 |
| WP_009880683 | 861 | DPV---DFLE---AKGYKE--V-RKDLIIK--LPKYSLFE---LENGRKRMLAS -GELQKGNELALPSKYVNFLYLA | 923 |
| WP_010922251 | 1177 | NPI---DFLE---AKGYKE--V-KKDLIIK--LPKYSLFE---LENGRKRMLAS -GELQKGNELALPSKYVNFLYLA | 1239 |
| WP_011054416 | 1177 | DPI---DFLE---AKGYKE--V-RKDLIIK--LPKYSLFE---LENGRKRMLAS -GELQKGNELALPSKYVNFLYLA | 1239 |
| WP_011284745 | 1177 | NPI---DFLE---AKGYKE--V-RKDLIVK--LPKYSLFE---LENGRKRMLAS -GELQKGNELALPSKYVNFLYLA | 1239 |
| WP_011285506 | 1177 | NPI---DFLE---AKGYKE--V-KKDLIIK--LPKYSLFE---LENGRKRMLAS -GELQKGNELALPSKYVNFLYLA | 1239 |
| WP_011527619 | 1177 | NPI---DFLE---AKGYKE--V-RKDLIIK--LPKYSLFE---LENGRKRMLAS -GELQKGNELALPSKYVNFLYLA | 1239 |
| WP_012560673 | 1177 | DPV---DFLE---AKGYKE--V-RKDLIIK--LPKYSLFE---LENGRKRMLAS -GELQKGNELALPSKYVNFLYLA | 1239 |
| WP_014407541 | 1176 | NPI---DFLE---AKGYKE--V-KKDLIIK--LPKYSLFE---LENGRKRMLAS -GELQKGNELALPSKYVNFLYLA | 1238 |
| WP_020905136 | 1177 | NPI---DFLE---AKGYKE--V-KKDLIIK--LPKYSLFE---LENGRKRMLAS -GELQKGNELALPSKYVNFLYLA | 1239 |
| WP_023080005 | 1176 | NPI---DFLE---AKGYKE--V-RKDLIIK--LPKYSLFE---LENGRKRMLAS -GELQKGNELALPSKYVNFLYLA | 1238 |
| WP_023610282 | 1176 | NPI---DFLE---AKGYKE--V-RKDLIIK--LPKYSLFE---LENGRKRMLAS -GELQKGNELALPSKYVNFLYLA | 1238 |
| WP_030125963 | 1177 | NPI---DFLE---AKGYKE--V-RKDLIIK--LPKYSLFE---LENGRKRMLAS -GELQKGNELALPSKYVNFLYLA | 1239 |
| WP_030126706 | 1177 | NPI---DFLE---AKGYKE--V-RKDLIIK--LPKYSLFE---LENGRKRMLAS -GELQKGNELALPSKYVNFLYLA | 1239 |
| WP_031488318 | 1177 | NPI---DFLE---AKGYKE--V-RKDLIIK--LPKYSLFE---LENGRKRMLAS -GELQKGNELALPSKYVNFLYLA | 1239 |
| WP_032460140 | 1177 | DPV---DFLE---AKGYKE--V-RKDLIIK--LPKYSLFE---LENGRKRMLAS -GELQKGNELALPSKYVNFLYLA | 1239 |
| WP_032461047 | 1177 | DPV---DFLE---AKGYKE--V-RKDLIIK--LPKYSLFE---LENGRKRMLAS -GELQKGNELALPSKYVNFLYLA | 1239 |
| WP_032462016 | 1177 | NPI---DFLE---AKGYKE--V-KKDLIIK--LPKYSLFE---LENGRKRMLAS -GELQKGNELALPSKYVNFLYLA | 1239 |
| WP_032462936 | 1177 | NPI---DFLE---AKGYKE--V-RKDLIIK--LPKYSLFE---LENGRKRMLAS -GELQKGNELALPSKYVNFLYLA | 1239 |
| WP_032464890 | 1177 | NPI---DFLE---AKGYKE--V-RKDLIIK--LPKYSLFE---LENGRKRMLAS -GELQKGNELALPSKYVNFLYLA | 1239 |
| WP_033888930 | 1002 | NPI---DFLE---AKGYKE--V-KKDLIIK--LPKYSLFE---LENGRKRMLAS -GELQKGNELALPSKYVNFLYLA | 1064 |
| WP_038431314 | 1177 | NPI---DFLE---AKGYKE--V-RKDLIIK--LPKYSLFE---LENGRKRMLAS -GELQKGNELALPSKYVNFLYLA | 1239 |
| WP_038432938 | 1176 | NPI---DFLE---AKGYKE--V-RKDLIIK--LPKYSLFE---LENGRKRMLAS -GELQKGNELALPSKYVNFLYLA | 1238 |
| WP_038434062 | 1177 | NPI---DFLE---AKGYKE--V-RKDLIIK--LPKYSLFE---LENGRKRMLAS -GELQKGNELALPSKYVNFLYLA | 1239 |
| BAQ51233 | 1088 | NPI---DFLE---AKGYKE--V-KKDLIIK--LPKYSLFE---LENGRKRMLAS -GELQKGNELALPSKYVNFLYLA | 1150 |
| KGE60162 | 352 | DPI---DFLE---AKGYKE--V-RKDLIIK--LPKYSLFE---LENGRKRMLAS -GELQKGNELALPSKYVNFLYLA | 414 |
| KGE60856 | 115 | DPI---DFLE---AKGYKE--V-RKDLIIK--LPKYSLFE---LENGRKRMLAS -GELQKGNELALPSKYVNFLYLA | 177 |
| WP_002989955 | 1177 | NPI---DFLE---AKGYKE--V-RKDLIIK--LPKYSLFE---LENGRKRMLAS -GELQKGNELALPSKYVNFLYLA | 1239 |
| WP_003030002 | 1159 | HPV---DFLE---QRGYRN--V-RLEKIIK--LPKYSLFE---LENKRRRLLAS ARELQKGNELVIPQRFTTLLYHS | 1221 |
| WP_003065552 | 1187 | NPV---EFLE---NKGYHN--I-REDKLIK--LPKYSLFE---FEGGKRRLLAS ASELQKGNEMVIPGHLVKLLYHA | 1249 |
| WP_001040076 | 1178 | NPS---AFLE---SKGYLN--I-RTDKLII--LPKYSLFE---LENGRRRLLAS AGELQKGNELALPTQFMKFLYLA | 1240 |
| WP_001040078 | 1186 | NPS---AFLE---SKGYLN--I-RDDKLMI--LPKYSLFE---LENGRRRLLAS AGELQKGNELALPTQFMKFLYLA | 1248 |
| WP_001040080 | 1178 | NPS---AFLE---SKGYLN--I-RADKLII--LPKYSLFE---LENGRRRLLAS AGELQKGNELALPTQFMKFLYLA | 1240 |
| WP_001040081 | 1178 | NPS---AFLE---SKGYLN--I-RADKLII--LPKYSLFE---LENGRRRLLAS AGETIDRLQKGNELALPTQFMKFLYLA | 1240 |
| WP_001040083 | 1178 | NPS---AFLE---SKGYLN--I-RADKLII--LPKYSLFE---LENGRRRLLAS AGELQKGNELALPTQFMKFLYLA | 1240 |
| WP_001040085 | 1178 | NPS---AFLE---SKGYLN--I-RADKLII--LPKYSLFE---LENGRRRLLAS AGELQKGNELALPTQFMKFLYLA | 1240 |
| WP_001040087 | 1178 | NPS---AFLE---SKGYLN--I-RADKLII--LPKYSLFE---LENGRRRLLAS AGELQKGNELALPTQFMKFLYLA | 1240 |
| WP_001040088 | 1178 | NPS---AFLE---SKGYLN--I-RADKLII--LPKYSLFE---LENGRRRLLAS AGELQKGNELALPTQFMKFLYLA | 1240 |
| WP_001040089 | 1178 | NPS---AFLE---SKGYLN--I-RDDKLMI--LPKYSLFE---LENGRRRLLAS AGELQKGNELALPTQFMKFLYLA | 1240 |
| WP_001040090 | 1178 | NPS---AFLE---SKGYLN--I-RADKLII--LPKYSLFE---LENGRRRLLAS AGELQKGNELALPTQFMKFLYLA | 1240 |
| WP_001040091 | 1178 | NPS---AFLE---SKGYLN--I-RADKLII--LPKYSLFE---LENGRRRLLAS AGELQKGNELALPTQFMKFLYLA | 1240 |
| WP_001040092 | 1178 | NPS---AFLE---SKGYLN--I-RTDKLII--LPKYSLFE---LENGRRRLLAS AGELQKGNELALPTQYMKFLYLA | 1240 |
| WP_001040094 | 1178 | NPS---AFLE---SKGYLN--I-RDDKLMI--LPKYSLFE---LENGRRRLLAS AGELQKGNELALPTQFMKFLYLA | 1240 |
| WP_001040095 | 1178 | NPS---AFLE---SKGYLN--I-RTDKLII--LPKYSLFE---LENGRRRLLAS AGELQKGNELALPTQYMKFLYLA | 1240 |
| WP_001040096 | 1178 | NPS---AFLE---SKGYLN--I-RTDKLII--LPKYSLFE---LENGRRRLLAS AGELQKGNELALPTQYMKFLYLA | 1240 |
| WP_001040097 | 1178 | NPS---AFLE---SKGYLN--I-RDDKLMI--LPKYSLFE---LENGRRRLLAS ADELQKGNELALPTQFMKFLYLA | 1240 |
| WP_001040098 | 1178 | NPS---AFLE---SKGYLN--I-RDDKLMI--LPKYSLFE---LENGRRRLLAS AGELQKGNELALPTQFMKFLYLA | 1240 |
| WP_001040099 | 1178 | NPS---AFLE---SKGYLN--I-RDDKLMI--LPKYSLFE---LENGRRRLLAS AGELQKGNELALPTQFMKFLYLA | 1240 |
| WP_001040100 | 1178 | NPS---AFLE---SKGYLD--I-RDDKLMI--LPKYSLFE---LENGRRRLLAS AGELQKGNELALPTQFMKFLYLA | 1240 |
| WP_001040104 | 1178 | NPS---AFLE---SKGYLN--I-RADKLII--LPKYSLFE---LENGRRRLLAS AGELQKGNELALPTQFMKFLYLA | 1240 |
| WP_001040105 | 1178 | NPS---AFLE---SKGYLN--I-RADKLII--LPKYSLFE---LENGRRRLLAS AGELQKGNELALPTQFMKFLYLA | 1240 |
| WP_001040106 | 1178 | NPS---AFLE---SKGYLN--I-RDDKLMI--LPKYSLFE---LENGRRRLLAS AGELQKGNELALPTQYMKFLYLA | 1240 |
| WP_001040107 | 1178 | NPS---AFLE---SKGYLN--I-RDDKLMI--LPKYSLFE---LENGRRRLLAS AGELQKGNELALPTQYMKFLYLA | 1240 |
| WP_001040108 | 1178 | NPS---AFLE---SKGYLN--I-RDDKLMI--LPKYSLFE---LENGRRRLLAS AGELQKGNELALPTQYMKFLYLA | 1240 |
| WP_001040109 | 1178 | NPS---AFLE---SKGYLN--I-RDDKLMI--LPKYSLFE---LENGRRRLLAS AGELQKGNELALPTQYMKFLYLA | 1240 |
| WP_001040110 | 1178 | NPS---AFLE---SKGYLN--I-RDDKLMI--LPKYSLFE---LENGRRRLLAS AGELQKGNELALPTQYMKFLYLA | 1240 |
| WP_015058523 | 1178 | NPS---AFLE---SKGYLN--I-RTDKLII--LPKYSLFE---LENGRRRLLAS AGELQKGNELALPTQYMKFLYLA | 1240 |
| WP_017643650 | 1178 | NPS---AFLE---SKGYLN--I-RDDKLMI--LPKYSLFE---LENGRRRLLAS ADELQKGNELALPTQFMKFLYLA | 1240 |
| WP_017647151 | 1178 | NPS---AFLE---SKGYLN--I-RDDKLMI--LPKYSLFE---LENGRRRLLAS AGELQKGNELALPTQFMKFLYLA | 1240 |
| WP_017648376 | 1178 | NPS---AFLE---SKGYLN--I-RDDKLMI--LPKYSLFE---LENGRRRLLAS AGELQKGNELALPTQFMKFLYLA | 1240 |
| WP_017649527 | 1178 | NPS---AFLE---SKGYLN--I-RADKLII--LPKYSLFE---LENGRRRLLAS AGELQKGNELALPTQFMKFLYLA | 1240 |
| WP_017771611 | 1178 | NPS---AFLE---SKGYLN--I-RDDKLMI--LPKYSLFE---LENGRRRLLAS AGELQKGNELALPTQYMKFLYLA | 1240 |
| WP_017771984 | 1178 | NPS---AFLE---SKGYLN--I-RADKLII--LPKYSLFE---LENGRRRLLAS AGELQKGNELALPTQFMKFLYLA | 1240 |
| CFQ25032 | 1178 | NPS---AFLE---SKGYLN--I-RADKLII--LPKYSLFE---LENGRRRLLAS AGELQKGNELALPTQFMKFLYLA | 1240 |
| CFV16040 | 1178 | NPS---AFLE---SKGYLN--I-RADKLII--LPKYSLFE---LENGRRRLLAS AGELQKGNELALPTQFMKFLYLA | 1240 |
| KLJ37842 | 1178 | NPS---AFLE---SKGYLN--I-RADKLII--LPKYSLFE---LENGRRRLLAS AGELQKGNELALPTQFMKFLYLA | 1240 |
| KLJ72361 | 1178 | NPS---AFLE---SKGYLN--I-RADKLII--LPKYSLFE---LENGRRRLLAS AGELQKGNELALPTQFMKFLYLA | 1240 |
| KLL20707 | 1192 | NPS---AFLE---SKGYLN--I-RADKLII--LPKYSLFE---LENGRRRLLAS AGELQKGNELALPTQFMKFLYLA | 1254 |
| KLL42645 | 1178 | NPS---AFLE---SKGYLN--I-RDDKLMI--LPKYSLFE---LENGRRRLLAS AGELQKGNELALPTQYMKFLYLA | 1240 |
| WP_047207273 | 1178 | NPS---AFLE---SKGYLN--I-RADKLII--LPKYSLFE---LENGRRRLLAS AGELQKGNELALPTQFMKFLYLA | 1240 |
| WP_047209694 | 1178 | NPS---AFLE---SKGYLN--I-RDDKLMI--LPKYSLFE---LENGRRRLLAS AGELQKGNELALPTQFMKFLYLA | 1240 |
| WP_050198062 | 1178 | NPS---AFLE---SKGYLN--I-RADKLII--LPKYSLFE---LENGRRRLLAS AGELQKGNELALPTQFMKFLYLA | 1240 |
| WP_050201642 | 1178 | NPS---AFLE---SKGYLN--I-RADKLII--LPKYSLFE---LENGRRRLLAS AGELQKGNELALPTQFMKFLYLA | 1240 |
| WP_050204027 | 1178 | NPS---AFLE---SKGYLN--I-RDDKLMI--LPKYSLFE---LENGRRRLLAS AGELQKGNELALPTQYMKFLYLA | 1240 |
| WP_050881965 | 1178 | NLS---AFLE---SKGYLN--I-RADKLII--LPKYSLFE---LENGRRRLLAS AGELQKGNELALPTQFMKFLYLA | 1240 |
| WP_050886065 | 1178 | NPS---AFLE---SKGYLN--I-RADKLII--LPKYSLFE---LENGRRRLLAS AGELQKGNELALPTQFMKFLYLA | 1240 |
| AHN30376 | 1178 | NPS---AFLE---SKGYLN--I-RTDKLII--LPKYSLFE---LENGRRRLLAS AGELQKGNELALPTQYMKFLYLA | 1240 |
| EAO78426 | 1178 | NPS---AFLE---SKGYLN--I-RADKLII--LPKYSLFE---LENGRRRLLAS AGELQKGNELALPTQFMKFLYLA | 1240 |
| CCW42055 | 1178 | NPS---AFLE---SKGYLN--I-RTDKLII--LPKYSLFE---LENGRRRLLAS AGELQKGNELALPTQFMKFLYLA | 1240 |
| WP_003041502 | 1186 | DKR---AFLL---GKGYKD--I-K--KIIE--LPKYSLFE---LKDGSRRMLAS RGEIHKGNELFVPQKFTTLLYHA | 1253 |
| WP_037593752 | 1160 | NPV---DFLE---QRGYRN--V-RLEKIIK--LPKYSLFE---LENKRRRLLAS ARELQKGNELVIPQRFTTLLYHS | 1222 |
| WP_049516684 | 1160 | HPV---DFLE---QRGYRN--V-RLEKIIK--LPKYSLFE---LENKRRRLLAS ARELQKGNELVIPQRFTTLLYHS | 1222 |
| GAD46167 | 1159 | NPV---DFLE---QRGYRN--V-RLEKIIK--LPKYSLFE---LENKRRRLLAS ARELQKGNELVIPQRFTTLLYHS | 1221 |
| WP_018363470 | 1191 | NPV---EFLK---NKGYQN--V-QEDKLMK--LPKYSLFE---FEGGRRRLLAS ATELQKGNEIMLSAHLVALLYHA | 1253 |
| WP_003043819 | 1186 | DPI---GFLE---AKGYKD--I-KKELIFK--LPKYSLFE---LENGRRRMLAS --ELQKANELVLPQHLVRLLYYT | 1248 |
| WP_006269658 | 1159 | NPV---DFLE---QRGYRN--V-RLEKIIK--LPKYSLFE---LENKRRRLLAS AKELQKGNELVIPQRFTTLLYHS | 1221 |
| WP_048800889 | 1179 | NPI---MFLE---SKGYRN--I-QKDKLIK--LPKYSLFE---FEGGRRRLLAS AVELQKGNEMVLPQYLNNLLYHA | 1241 |
| WP_012767106 | 1178 | NPL---KFIE---DKGYGN--V-QIDKCIK--LPKYSLFE---FENGTRRMLAS RGDLQKANEMFLPAKLVTLLY-- | 1245 |
| WP_014612333 | 1178 | NPL---KFIE---DKGYGN--V-QIDKCIK--LPKYSLFE---FENGTRRMLAS RGDLQKANEMFLPAKLVTLLY-- | 1245 |
| WP_015017095 | 1178 | NPL---KFIE---DKGYGN--V-QIDKCIK--LPKYSLFE---FENGTRRMLAS RGDLQKANEMFLPAKLVTLLY-- | 1245 |
| WP_015057649 | 1178 | NPL---KFIE---DKGYGN--V-QIDKCIK--LPKYSLFE---FENGTRRMLAS RGDLQKANEMFLPAKLVTLLY-- | 1245 |
| WP_048327215 | 1178 | NPL---KFIE---DKGYGN--V-QIDKCIK--LPKYSLFE---FENGTRRMLAS RGDLQKANEMFLPAKLVTLLY-- | 1245 |
| WP_049519324 | 1178 | NPL---KFIE---DKGYGN--V-QIDKCIK--LPKYSLFE---FENGTRRMLAS RGDLQKANEMFLPAKLVTLLY-- | 1245 |
| WP_012515931 | 1157 | NPV---VFLE---ARGYRE--I-QEHLIIK--LPKYSLFE---LENGRRRLLAS -SELQKGNELFLPVDYMTFLYLA | 1219 |
| WP_021320964 | 1157 | NPV---VFLE---AKGYRE--I-QEHLIIK--LPKYSLFE---LENGRRRLLAS -SELQKGNELFLPVDYMTFLYLA | 1219 |
| WP_037581760 | 1157 | NPV---VFLE---AKGYRE--I-QEHLIIK--LPKYSLFE---LENGRRRLLAS -SELQKGNELFLPVDYMTFLYLA | 1219 |
| WP_004232481 | 1189 | NPV---SFLE---KKGYHN--V-QEDKLIK--LPKYSLFE---FEGGRRRLLAS ATELQKGNEVVLPQYMVNLLYHS | 1251 |
| WP_009854540 | 1184 | NPV---EFLE---NKGYHN--I-REDKLIK--LPKYSLFE---FEGGRRRLLAS ASELQKGNEMVLPGYLVELLYHA | 1246 |
| WP_012962174 | 1185 | NPV---VFLE---KKGYQN--V-QEDNLIK--LPKYSLFE---FEGGRRRLLAS ASELQKGNEVVLSRHLVELLYHA | 1247 |
| WP_039695303 | 1186 | NPV---EFLE---NKGYHN--I-REDKLIK--LPKYSLFE---FEGGRRRLLAS ASELQKGNEMVLPGYLVELLYHA | 1248 |
| WP_014334983 | 1189 | NPV---SFLE---KKGYHN--V-QEDKLIK--LPKYSLFE---FEGGRRRLLAS ATELQKGNEVMLPAHLVELLYHA | 1251 |
| WP_003099269 | 1177 | DPI---AFLE---KKGYQD--I-QTSSIIK--LPKYSLFE---LENGRKRLLAS --ELQKGNELALPNKYVKFLYLA | 1239 |
| AHY15608 | 1177 | DPI---AFLE---KKGYQD--I-QTSSIIK--LPKYSLFE---LENGRKRLLAS --ELQKGNELALPNKYVKFLYLA | 1239 |
| AHY17476 | 1177 | DPI---AFLE---KKGYQD--I-QTSSIIK--LPKYSLFE---LENGRKRLLAS --ELQKGNELALPNKYVKFLYLA | 1239 |
| ESR09100 | 9 | DPI---AFLE---KKGYQD--I-QTSSIIK--LPKYSLFE---LENGRKRLLAS -KELQKGNELALPNKYVKFLYLA | 71 |
| AGM98575 | 1177 | DPI---AFLE---KKGYQD--I-QTSSIIK--LPKYSLFE---LENGRKRLLAS --ELQKGNELALPNKYVKFLYLA | 1239 |
| ALF27331 | 1159 | NPV---AFLE---RKGYRN--V-QEENIVK--LPKYSLFE---LENGRRRLLAS ARELQKGNEIVLPNHLGTMLYHA | 1221 |
| WP_018372492 | 1168 | EPEr---FLA---QKGFER--V-EKT--IK--LPKYSLFE---MEKGRRRLLAS SGELQKGNQVLLPEHLIRLLSYA | 1228 |
| WP_045618028 | 1189 | NPI---AYLE---ECGYKN--I-NPNLIIK--LPKYSLFE---FNNGQRRLLAS SIELQKGNELIVPYHFTALLYHA | 1251 |
| WP_045635197 | 1184 | NPI---TFLE---NKGYHN--V-RKENILC--LPKYSLFE---LENGRRRLLAS AKELQKGNEIVLPVYLTTLLYHS | 1246 |
| WP_002263549 | 1159 | DPV---AFLE---RKGYRN--V-QEENIIK--LPKYSLFK---LENGRKRLLAS ARELQKGNEIVLPNHLGTLLYHA | 1221 |
| WP_002263887 | 1159 | DPV---AFLE---RKGYRN--V-QEENIIK--LPKYSLFK---LENGRKRLLAS ARELQKGNEIVLPNHLGTLLYHA | 1221 |
| WP_002264920 | 1159 | DPV---AFLE---RKGYRN--V-QEENIIK--LPKYSLFK---LENGRKRLLAS ARELQKGNEIVLPNHLGTLLYHA | 1221 |
| WP_002269043 | 1159 | DPV---AFLE---RKGYRN--V-QEENIIK--LPKYSLFK---LENGRKRLLAS ARELQKGNEIVLPNHLGTLLYHA | 1221 |
| WP_002269448 | 1159 | DPV---AFLE---RKGYRN--V-QEENIIK--LPKYSLFK---LENGRKRLLAS ARELQKGNEIVLPNHLGTLLYHA | 1221 |
| WP_002271977 | 1159 | DPV---AFLE---RKGYRN--V-QEENIIK--LPKYSLFK---LENGRKRLLAS ARELQKGNEIVLPNHLGTLLYHA | 1221 |
| WP_002272766 | 1159 | DPV---AFLE---RKGYRN--V-QEENIIK--LPKYSLFK---LENGRKRLLAS ARELQKGNEIVLPNHLGTLLYHA | 1221 |
| WP_002273241 | 1159 | DPV---AFLE---RKGYRN--V-QEENIIK--LPKYSLFK---LENGRKRLLAS ARELQKGNEIVLPNHLGTLLYHA | 1221 |
| WP_002275430 | 1159 | DPV---AFLE---RKGYRN--V-QEENIIK--LPKYSLFK---LENGRKRLLAS ARELQKGNEIVLPNHLGTLLYHA | 1221 |
| WP_002276448 | 1159 | DPV---AFLE---RKGYRN--V-QEENIIK--LPKYSLFK---LENGRKRLLAS ARELQKGNEIVLPNHLGTLLYHA | 1221 |
| WP_002277050 | 1167 | NPV---KFLK---DKGYQQ--I-EKNNFVK--LPKYTLVD---IGNGIKRLWAS SKEVHKGNOLVVSKKSQDLLYHA | 1229 |
| WP_002277364 | 1159 | DPV---AFLE---RKGYRN--V-QEENIIK--LPKYSLFK---LENGRKRLLAS ARELQKGNEIVLPNHLGTLLYHA | 1221 |
| WP_002279025 | 1159 | DPV---AFLE---RKGYRN--V-QEENIIK--LPKYSLFK---LENGRKRLLAS ARELQKGNEIVLPNHLGTLLYHA | 1221 |
| WP_002279859 | 1159 | DPV---AFLE---RKGYRN--V-QEENIIK--LPKYSLFK---LENGRKRLLAS ARELQKGNEIVLPNHLGTLLYHA | 1221 |
| WP_002280230 | 1159 | DPV---AFLE---RKGYRN--V-QEENIIK--LPKYSLFK---LENGRKRLLAS ARELQKGNEIVLPNHLGTLLYHA | 1221 |
| WP_002281696 | 1159 | DPV---AFLE---RKGYRN--V-QEENIIK--LPKYSLFK---LENGRKRLLAS ARELQKGNEIVLPNHLGTLLYHA | 1221 |
| WP_002282247 | 1167 | NPV---KFLK---DKGYQQ--I-EKNNFVK--LPKYTLVD---IGNGIKRLWAS SKEVHKGNQLVVSKKSQDLLYHA | 1229 |
| WP_002282906 | 1159 | DPV---AFLE---RKGYRN--V-QEENIIK--LPKYSLFK---LENGRKRLLAS ARELQKGNEIVLPNHLGTLLYHA | 1221 |
| WP_002283846 | 1159 | DPV---AFLE---RKGYRN--V-QEENIIK--LPKYSLFK---LENGRKRLLAS ARELQKGNEIVLPNHLGTLLYHA | 1221 |
| WP_002287255 | 1159 | DPV---AFLE---RKGYRN--V-QEENIIK--LPKYSLFK---LENGRKRLLAS ARELQKGNEIVLPNHLGTLLYHA | 1221 |
| WP_002288990 | 1159 | DPV---AFLE---RKGYRN--V-QEENIIK--LPKYSLFK---LENGRKRLLAS ARELQKGNEIVLPNHLGTLLYHA | 1221 |
| WP_002289641 | 1159 | DPV---AFLE---RKGYRN--V-QEENIIK--LPKYSLFK---LENGRKRLLAS ARELQKGNEIVLPNHLGTLLYHA | 1221 |
| WP_002290427 | 1159 | DPV---AFLE---RKGYRN--V-QEENIIK--LPKYSLFK---LENGRKRLLAS ARELQKGNEIVLPNHLGTLLYHA | 1221 |
| WP_002295753 | 1159 | DPI---AFLE---RKGYRN--V-QEENIIK--LPKYSLFK---LENGRKRLLAS ARELQKGNEIVLPNHLGTLLYHA | 1221 |
| WP_002296423 | 1159 | DPV---AFLE---RKGYRN--V-QEENIIK--LPKYSLFK---LENGRKRLLAS ARELQKGNEIVLPNHLGTLLYHA | 1221 |
| WP_002304487 | 1173 | DPV---AFLE---RKGYRN--V-QEENIIK--LPKYSLFK---LENGRKRLLAS ARELQKGNEIVLPNHLETLLYHA | 1235 |
| WP_002305844 | 1159 | DPV---AFLE---RKGYRN--V-QEENIIK--LPKYSLFK---LENGRKRLLAS ARELQKGNEIVLPNHLGTLLYHA | 1221 |
| WP_002307203 | 1159 | DPI---AFLE---RKGYRN--V-QEENIIK--LPKYSLFK---LENGRKRLLAS ARELQKGNEIVLPNHLGTLLYHA | 1221 |
| WP_002310390 | 1159 | DPV---AFLE---RKGYRN--V-QEENIIK--LPKYSLFK---LENGRKRLLAS ARELQKGNEIVLPNHLGTLLYHA | 1221 |
| WP_002352408 | 1159 | DPV---AFLE---RKGYRN--V-QEENIIK--LPKYSLFK---LENGRKRLLAS ARELQKGNEIVLPDHLGTLLYHA | 1221 |
| WP_012997688 | 1159 | DPV---AFLE---RKGYRN--V-QEENIIK--LPKYSLFK---LENGRKRLLAS ARELQKGNEIVLPNHLGTLLYHA | 1221 |
| WP_014677909 | 1159 | DPV---AFLE---RKGYRN--V-QEENIIK--LPKYSLFK---LENGRKRLLAS ARELQKGNEIVLPNHLGTLLYHA | 1221 |
| WP_019312892 | 1159 | DPV---AFLE---RKGYRN--V-QEENIIK--LPKYSLFK---LENGRKRLLAS ARELQKGNEIVLPNHLGTLLYHA | 1221 |
| WP_019313659 | 1159 | DPV---AFLE---RKGYRN--V-QEENIIK--LPKYSLFK---LENGRKRLLAS ARELQKGNEIVLPNHLGTLLYHA | 1221 |
| WP_019314093 | 1159 | DPI---AFLE---RKGYRN--V-QEENIIK--LPKYSLFK---LENGRKRLLAS ARELQKGNEIVLPNHLGTLLYHA | 1221 |
| WP_019315370 | 1159 | DPV---AFLE---RKGYRN--V-QEENIIK--LPKYSLFK---LENGRKRLLAS ARELQKGNEIVLPNHLGTLLYHA | 1221 |
| WP_019803776 | 1159 | DPV---AFLE---RKGYRN--V-QEENIIK--LPKYSLFK---LENGRKRLLAS ARELQKGNEIVLPNHLGTLLYHA | 1221 |
| WP_019805234 | 1159 | DPV---AFLE---RKGYRN--V-QEENIIK--LPKYSLFK---LENGRKRLLAS ARELQKGNEIVLPNHLGTLLYHA | 1221 |
| WP_024783594 | 1159 | DPV---AFLE---RKGYRN--V-QEENIIK--LPKYSLFK---LENGRKRLLAS ARELQKGNEIVLPNHLGTLLYHA | 1221 |
| WP_024784288 | 1167 | NPV---KFLK---DKGYQQ--I-EKNNFVK--LPKYTLVD---IGNGIKRLWAS SKEVHKGNQLVVSKKSQDLLYHA | 1229 |
| WP_024784666 | 1159 | DPV---AFLE---RKGYRN--V-QEENIIK--LPKYSLFK---LENGRKRLLAS ARELQKGNEIVLPNHLGTLLYHA | 1221 |
| WP_024784894 | 1159 | DPV---AFLE---RKGYRN--V-QEENIIK--LPKYSLFK---LENGRKRLLAS ARELQKGNEIVLPNHLGTLLYHA | 1221 |
| WP_024786433 | 1167 | NPV---KFLK---DKGYQQ--I-EKNNFVK--LPKYTLVD---IGNGIKRLWAS SKEVHKGNQLVVSKKSQDLLYHA | 1229 |
| WP_049473442 | 1159 | DPV---AFLE---RKGYRN--V-QEENIIK--LPKYSLFK---LENGRKRLLAS ARELQKGNEIVLPNHLGTLLYHA | 1221 |
| WP_049474547 | 1159 | DPV---AFLE---RKGYRN--V-QEENIIK--LPKYSLFK---LENGRKRLLAS ARELQKGNEIVLPNHLGTLLYHA | 1221 |
| EMC03581 | 1152 | DPV---AFLE---RKGYRN--V-QEENIIK--LPKYSLFK---LENGRKRLLAS ARELQKGNEIVLPNHLGTLLYHA | 1214 |
| WP_000428612 | 1187 | SPI---AFLE---NKGYHN--V-RKENILC--LPKYSLFE---LKNGRRRMLAS AKELQKGNEIVLPVHLTTLLYHA | 1249 |
| WP_000428613 | 1185 | NPI---TFLE---NKGYHN--V-RKENILC--LPKYSLFE---LENGRRRLLAS AKELQKGNEIVLPVYLTTLLYHS | 1247 |
| WP_049523028 | 1180 | NPV---AFLE---GKGYQN--V-VEENIIR--LPKYSLFE---LENGRRRMLAS AKELQKGNEMVLPSYLIALLYHA | 1242 |
| WP_003107102 | 1146 | DRI---TFLE---KKGYQD--I-QESLIIK--LPKFSLFE---LENGRKRLLAS --ELQKGNELSLPNKYIQFLYLA | 1208 |
| WP_054279288 | 1179 | NPI---AFLE---SKGYHD--I-QEHLMIT--LPKYSLFE---LENGRRRLLAS --ELQKGNEMVLPQHLVTFLYRV | 1241 |
| WP_049531101 | 1189 | NPT---AYLE---EYGYKN--I-NPNLIIK--LPKYSLFK---ENDGORRLLAS SIELQKGNELILPYHFTTLLYHA | 1251 |
| WP_049538452 | 1189 | NPI---AYLE---ECGYKN--I-NPNLIIK--LPKYSLFE---FNGGORRLLAS SIELQKGNELILPYHFTALLYHT | 1251 |
| WP_049549711 | 1191 | NPI---AYLE---ECGYKN--I-NPNLIIK--LPKYSLFE---FNGGQRRLLAS SIELQKGNELILPYHFTALLYHA | 1253 |
| WP_007896501 | 1183 | DNI---AFLE---KKGYQD--I-QEKLLIK--LPKYSLFE---LENGRRRLLAS --EFQKGNELALSGKYMKFLYLA | 1245 |
| EFR44625 | 1135 | DNI---AFLE---KKGYQD--I-QEKLLIK--LPKYSLFE---LENGRRRLLAS --EFQKGNELALSGKYMKFLYLA | 1197 |
| WP_002897477 | 1184 | NPI---TFLE---NKGYHN--V-RKENILC--LPKYSLFE---LENGRRRLLAS AKELQKGNEIVLPVCLTTLLYHS | 1246 |
| WP_002906454 | 1190 | NPV---TYLE---ECGYKN--I-NSNLIIK--LPKYSLFE---ENDGORRLLAS SIELQKGNELILPYHLTALLYHA | 1252 |
| WP_009729476 | 1185 | NPI---AFLE---NKGYHN--V-CKENILC--LPKYSLFE---LENGRRRLLAS AKELQKCNEIVLPVYLITLLYHS | 1247 |
| COR24647 | 1175 | NSV---TFLE---EKGYRN--I-RENTIIK--FPKYSLFE---LENGRRRLLAS AIELQKGNEMFLPQQFVNLLYHA | 1237 |
| WP_000066813 | 1189 | NPI---TFLE---NKGYHN--V-RKENILC--LPKYSLFE---LESGRRRMLAS AKELQKGNEIVLPVYLTTLLYHS | 1251 |
| WP_009754323 | 1185 | NPI---TFLE---NKGYHN--V-RKENILC--LPKYSLFE---LENGRRRLLAS AKELQKGNEIVLPVYLTTLLYHS | 1247 |
| WP_044674937 | 1178 | NPI---EFLE---HKGYKN--I-LEKNIIK--LPKYSLFE---LENGRRRLLAS AKELQKGNEMILPPHLVTLLYHS | 1240 |
| WP_044676715 | 1180 | NPI---EFLE---HKGYKN--I-LEKNIIK--LPKYSLFE---LENGRRRLLAS AKELQKGNEMILPPHLVTLLYHS | 1242 |
| WP_044680361 | 1180 | NPI---EFLE---HKGYKN--I-LEKNIIK--LPKYSLFE---LENGRRRLLAS AKELQKGNEMILPPHLVTLLYHS | 1242 |
| WP_044681799 | 1178 | NPI---EFLE---HKGYKN--I-LEKNIIK--LPKYSLFE---LENGRRRLLAS AKELQKGNEMILPPHLVTLLYHS | 1240 |
| WP_049533112 | 1186 | DKR---AFLL---GKGYKD--I-K--KIIE--LPKYSLFE---LKDGSRRMLAS RGEIHKGNELFVPQKFTTLLYHA | 1253 |
| WP_029090905 | 1118 | SETslqAYIA---EQIKSE--VeILN----grILKYQLIS----NNGNRLYIAG -SERHNARQLIVSDEAAKVIWLI | 1181 |
| WP_006506696 | 1150 | EKI---NYIE--eKEGLSD--VrIIK---Dn-IPVNOMIEm----DGGEYLLTS --EYVNARQLVLNEKQCALIADI | 1211 |
| AIT42264 | 1177 | NPI---DFLE---AKGYKE--V-KKDLIIK--LPKYSLFE---LENGRKRMLAS -GELQKGNELALPSKYVNFLYLA | 1239 |
| WP_034440723 | 1150 | QTT---EYLG---KIGFNK--ASIIN---S--FKNYTLFE---LENGSRRMIVG KGELQKGNQMYLPQNLLEFVYHL | 1217 |
| AKQ21048 | 1177 | NPI---DFLE---AKGYKE--V-KKDLIIK--LPKYSLFE---LENGRKRMLAS -GELQKGNELALPSKYVNFLYLA | 1239 |
| WP_004636532 | 1151 | HPT---AYLE---EAGYNN--P-TV--LHE--LFKYQLFE---LEDGSRRMIAS AKEFQKGNQMVLPLELVELLYHA | 1211 |
| WP_002364836 | 1157 | NPI---LFLE---EKGFLR--P-RV--LMK--LPKYTLYE---FPEGRRRLLAS AKEAQKGNQMVLPEHLLTLLYHA | 1217 |
| WP_016631044 | 1108 | NPI---LFLE---EKGFLR--P-RV--LMK--LPKYTLYE---FPEGRRRLLAS AKEAQKGNQMVLPEHLLTLLYHA | 1168 |
| EMS75795 | 893 | DPI---GFLS---NKGYSN--V-TKF--IK--LSKYTLYE---LENGRRRMVAS -KEAQKANSFILPEKLVTLLYHA | 953 |
| WP_002373311 | 1157 | NPI---LFLE---EKGFLR--P-RV--LMK--LPKYTLYE---FPEGRRRLLAS AKEAQKGNQMVLPEHLLTLLYHA | 1217 |
| WP_002378009 | 1157 | NPI---LFLE---EKGFLR--P-RV--LMK--LPKYTLYQ---FPEGRRRLLAS AKEAQKGNQMVLPEHLLTLLYHA | 1217 |
| WP_002407324 | 1157 | NPI---LFLE---EKGFLR--P-RV--LMK--LPKYTLYE---FPEGRRRLLAS AKEAQKGNQMVLPEHLLTLLYHA | 1217 |
| WP_002413717 | 1157 | NPI---LFLE---EKGFLR--P-RV--LMK--LPKYTLYE---FPEGRRRLLAS AKEAQKGNQMVLPEHLLTLLYHA | 1217 |
| WP_010775580 | 1159 | NPI---LFLE---EKGFLR--P-RV--LMK--LPKYTLYE---FPEGRRRLLAS AKEAQKGNQMVLPEHLLILLYHA | 1219 |
| WP_010818269 | 1157 | NPI---LFLE---EKGFLR--P-RV--LMK--LPKYTLYE---FPEGRRRLLAS AKEAQKGNQMVLPEHLLTLLYHA | 1217 |
| WP_010824395 | 1157 | NPI---LFLE---EKGFLR--P-RV--LMK--LPKYTLYE---FPEGRRRLLAS AKEAQKGNOMVLPEHLLTLLYHA | 1217 |
| WP_016622645 | 1157 | NPI---LFLE---EKGFLR--P-RV--LMK--LPKYTLYE---FPEGRRRLLAS AKEAQKGNQMVLPEHLLTLLYHA | 1217 |
| WP_033624816 | 1157 | NPI---LFLE---EKGFLR--P-RV--LMK--LPKYTLYE---FPEGRRRLLAS AKEAQKGNQMVLPERLLTLLYHA | 1217 |
| WP_033625576 | 1157 | NPI---LFLE---EKGFLR--P-RV--LMK--LPKYTLYE---FPEGRRRLLAS AKEAQKGNQMVLPEHLLTLLYHA | 1217 |
| WP_033789179 | 1157 | NPI---LFLE---EKGFLR--P-RV--LMK--LPKYTLYE---FPEGRRRLLAS AKEAQKGNQMVLPEHLLTLLYHA | 1217 |
| WP_002310644 | 1159 | SPV---LFLK---NKGYEQ--A-EIE--MK--LPKYALFE---LENGRKRMVAS -KEAQKANSFLLPEHLVTLLYHA | 1219 |
| WP_002312694 | 1160 | SPV---LFLK---NKGYEQ--A-EIE--MK--LPKYALFE---LENGRKRMVAS -KEAQKANSFLLPEHLVTLLYHA | 1220 |
| WP_002314015 | 1160 | SPV---LFLK---NKGYEQ--A-EIE--MK--LPKYALFE---LENGRKRMVAS -KEAQKANSELLPEHLVTLLYHA | 1220 |
| WP_002320716 | 1160 | SPV---LFLK---NKGYEQ--A-EIE--MK--LPKYALFE---LENGRKRMVAS -KEAQKANSELLPEHLVTLLYHA | 1220 |
| WP_002330729 | 1159 | SPV---LFLK---NKGYEQ--A-EIE--MK--LPKYALFE---LENGRKRMVAS -KEAQKANSELLPEHLVTLLYHA | 1219 |
| WP_002335161 | 1160 | SPV---LFLK---NKGYEQ--A-EIE--MK--LPKYALFE---LENGRKRMVAS -KEAQKANSFLLPEHLVTLLYHA | 1220 |
| WP_002345439 | 1160 | SPV---LFLK---NKGYEQ--A-EIE--MK--LPKYALFE---LENGRKRMVAS -KEAQKANSFLLPEHLVILLYHA | 1220 |
| WP_034867970 | 1150 | DPT---TFLK---EKGFPQ--V-TEF--IK--LPKYTLFE---FDNGRRRFLAS -KESQKGNPFILSDQLVTLLYHA | 1210 |
| WP_047937432 | 1160 | SPV---LFLK---NKGYEQ--A-EIE--MK--LPKYALFE---LENGRKRMVAS -KEAQKANSFLLPEHLVTLLYHA | 1220 |
| WP_010720994 | 1150 | DPT---TFLK---DKGFPQ--V-TEF--IK--LPKYTLFE---FDNGRRRFLAS -KESQKGNPFILSDQLVTLLYHA | 1210 |
| WP_010737004 | 1150 | DPT---TFLK---EKGFPQ--V-TEF--IK--LPKYTLFE---FDNGRRRFLAS -KESQKGNPFILSDQLVTLLYHA | 1210 |
| WP_034700478 | 1150 | DPT---TFLK---DKGFPH--V-TEF--IK--LPKYTLFE---FDNGRRRFLAS -KESQKGNPFILSDQLVTLLYHA | 1210 |
| WP_007209003 | 1148 | NPIl---YLS---KKDYHN---pKVEAI----LPKYSLFE---FENGRRRMVAS -SETQKGNOLIIPGHLMELLYHS | 1208 |
| WP_023519017 | 1144 | DPR---EFLK---TKGYEG--V-KQW--LI--LPKYILFE---AQGGYRRMIAS -QETQKANSLILPENLVTLLYHA | 1204 |
| WP_010770040 | 1155 | DPV---SLLE---EKGYAN--P-EV--LIH--LPKYTLYE---LENGRRRLLAS ANEAQKGNQLVLPASLVTLLYHA | 1215 |
| WP_048604708 | 1152 | NER---EFLK---NKGYQN--P-QI--CMK--LPKYSLYE---FDDGRRRLLAS AKEAQKGNQMVLPAHLVTFLYHA | 1212 |
| WP_010750235 | 1153 | DPI---SFLI---EKGYSN--V-NQF--IK--LPKYTLFE---LANGORRMLAS -QELQKANSFILPEKLVTLLYHA | 1213 |
| AII16583 | 1216 | NPI---DFLE---AKGYKE--V-KKDLIIK--LPKYSLFE---LENGRKRMLAS -GELQKGNELALPSKYVNFLYLA | 1278 |
| WP_029073316 | 1165 | TKI---NYIK--eSEGLEE--VKIIK---E--ILKNQLIEi----NGGLFYVTS --EIVNARQLILDENCTRIIDGI | 1225 |
| WP_031589969 | 1165 | IKI---NYLK--qAEDLEE--VqIGK---E--ILKNQLIEk----DGGLYYIVA --EIINAKQLILNESQTKLVCEI | 1225 |
| KDA45870 | 1139 | DPT---AYLA---SRGYTNvtNsFIL-------PKYSLLEd---PEGRRRYLAS -KEFQKANELILPQHLVELLYWV | 1199 |
| WP_039099354 | 1171 | QKI-spQFTKv---KKQKGtiV-KVVEDFEv-IAPHILINqrfFDNGQELTLGS ----HNEQELILDKTAVKLLNGA | 1241 |
| AKP02966 | 1173 | KTL--qNWLE---ENVKHKksIqIIK---Nn-VPIGQIIY------SKKVGLLS -REIANRQQLILPPEHSALLRIL | 1237 |
| WP_010991369 | 1155 | DEK---AFLE---EQGYRQ--P-KV--LAK--LPKYTLYE---CEEGRRRMLAS ANEAQKGNQQVLPNHLVTLLHHA | 1215 |
| WP_033838504 | 1155 | DEK---AFLE---EQGYRQ--P-KV--LAK--LPKYTLYE---CEEGRRRMLAS ANEAQKGNQQVLPNHLVTLLHHV | 1215 |
| EHN60060 | 1158 | DEK---AFLE---EQGYRQ--P-KV--LAK--LPKYTLYE---CEEGRRRMLAS ANEAQKGNQQVLPNHLVTLLHHV | 1218 |
| EFR89594 | 924 | DEK---AFLE---EQGYRQ--P-KV--LAK--LPKYTLYE---CEEGRRRMLAS ANEAQKGNQQVLPNHLVTLLHHA | 984 |
| WP_038409211 | 1155 | DQK---AFLE---EKGYYS--P-KV--LTK--IPKYTLYE---CENGRRRMLGS ANEAQKGNQMVLPNHLMILLYHA | 1215 |
| EFR95520 | 774 | DQK---AFLE---EKGYYS--P-KV--LTK--IPKYTLYE---CENGRRRMLGS ANEAQKGNQMVLPNHLMTLLYHA | 834 |
| WP_003723650 | 1155 | DEE---AFLE---EKGYRH--P-KV--LTK--LPKYTLYE---CEKGRRRMLAS ANEAQKGNQLVLSNHLVSLLYHA | 1215 |
| WP_003727705 | 1155 | DEE---AFLE---EKGYHQ--P-KV--LTK--LPKYTLYE---CEKGRRRMLSS ANEAQKGNQLVLSNHLVSLLYHA | 1215 |
| WP_003730785 | 1155 | DEE---AFLE---EKGYHQ--P-KV--LTK--LPKYTLYE---CEKGRRRMLSS ANEAQKGNQLVLSNHLVSLLYHA | 1215 |
| WP_003733029 | 1155 | DEK---TFLE---EKGYHQ--P-KV--LIK--VPKYTLYE---CKNGRRRMLGS ANEAHKGNQMLLPNHLMALLYHA | 1215 |
| WP_003739838 | 1155 | DEK---SFLE---KQGYRQ--P-KV--LTK--LPKYTLYE---CENGRRRMLAS ANEAQKGNQQVLKGOLITLLHHA | 1215 |
| WP_014601172 | 1155 | DEE---AFLE---EKGYRH--P-KV--LTK--LPKYTLYE---CEKGRRRMLAS ANEAQKGNQLVLSNHLVSLLYHA | 1215 |
| WP_023548323 | 1155 | DEK---VFLE---GKGYHQ--P-KV--LTK--LPKYALYE---CENGRRRMLGS ANEVHKGNQMLLPNHLMTLLYHA | 1215 |
| WP_031665337 | 1155 | DEE---AFLE---EKGYRH--P-KV--LTK--LPKYTLYE---CEKGRRRMLAS ANEAQKGNQLVLSNHLVSLLYHA | 1215 |
| WP_031669209 | 1155 | DEK---TFLE---EKGYHQ--P-KV--LIK--VPKYTLYE---CENGRRRMLGS ANEAHKGNQMLLPNHLMALLYHA | 1215 |
| WP_033920898 | 1155 | DEK---VFLE---GKGYHQ--P-KV--LTK--LPKYALYE---CENGRRRMLGS ANEVHKGNQMLLPNHLMTLLYHA | 1215 |
| AKI42028 | 1158 | DEE---AFLE---EKGYRH--P-KV--LTK--LPKYTLYE---CEKGRRRMLAS ANEAQKGNQLVLSNHLVSLLYHA | 1218 |
| AKI50529 | 1158 | DEK---VFLE---GKGYHQ--P-KV--LTK--LPKYALYE---CENGRRRMLGS ANEVHKGNQMLLPNHLMTLLYHA | 1218 |
| EFR83390 | 603 | DEE---AFLE---EKGYRH--P-KV--LTK--LPKYTLYE---CEKGRRRMLAS ANEAQKGNQLVLSNHLVSLLYHA | 663 |
| WP_046323366 | 1155 | DOK---EFLE---GKGYRN--P-KV--ITK--IPKYTLYE---CENGRRRMLGS ANEAQKGNQMVLPNHLMTLLYHA | 1215 |
| AKE81011 | 1193 | NPI---DFLE---AKGYKE--V-KKDLIIK--LPKYSLFE---LENGRKRMLAS -GELQKGNELALPSKYVNFLYLA | 1255 |
| CUO82355 | 1154 | EQI---EYVE--kEEKLSD--VKIIK---Nn-IPLNQLIEi----DGRQYLLTS --ECVNAMQLVLNEEQCKLIADI | 1215 |
| WP_033162887 | 1155 | EQL---SYIAspeHEDLID--VrIVK---E--ILKNQLIEi----DGGLYYVTS --EYVTARQLSLNEQSCKLISEI | 1217 |
| AGZ01981 | 1210 | NPI---DFLE---AKGYKE--V-KKDLIIK--LPKYSLFE---LENGRKRMLAS -GELQKGNELALPSKYVNFLYLA | 1272 |
| AKA60242 | 1177 | NPI---DFLE---AKGYKE--V-KKDLIIK--LPKYSLFE---LENGRKRMLAS -GELQKGNELALPSKYVNFLYLA | 1239 |
| AKS40380 | 1177 | NPI---DFLE---AKGYKE--V-KKDLIIK--LPKYSLFE---LENGRKRMLAS -GELQKGNELALPSKYVNFLYLA | 1239 |
| 4UN5_B | 1181 | NPI---DFLE---AKGYKE--V-KKDLIIK--LPKYSLFE---LENGRKRMLAS -GELQKGNELALPSKYVNFLYLA | 1243 |
| WP_010922251 | 1240 | SHYEKLKgSPEDnEQKOL--FVEQHKHYLDEIIEQISEFSKRVILAD--ANLDKVL-S AYN-KH---RDKPIREq- | 1305 |
| WP_039695303 | 1249 | HRAD----NFNS-TEYLN--YVSEHKKEFEKVLSCVEDFANLYVDVE--KNLSKIR-A VAD-SM---DNFSIEE-- | 1308 |
| WP_045635197 | 1247 | KNVH----KLDE-PGHLE--YIQKHRNEFKDLLNLVSEFSQKYVLAD--ANLEKIK-S LYA-DN---EQADIEI-- | 1306 |
| 5AXW_A | 979 | GELYRVIgVNND1LNRIE---VNMIDITYREYLENMNDKRPPRIIKTiaSKTQSIK-K LYEvKSk--KHPQIIKkg | 1056 |
| WP_009880683 | 924 | SHYEKLKgSPEDnEQKOL--FVEQHKHYLDEIIEQISEFSKRVILAD--ANLDKVL-S AYN-KH---RDKPIREq- | 989 |
| WP_010922251 | 1240 | SHYEKLKgSPEDnEQKOL--FVEQHKHYLDEIIEQISEFSKRVILAD--ANLDKVL-S AYN-KH---RDKPIREq- | 1305 |
| WP_011054416 | 1240 | SHYEKLKgSPEDnEQKOL--FVEQHKHYLDEIIEQISEFSKRVILAD--ANLDKVL-S AYN-KH---RDKPIREq- | 1305 |
| WP_011284745 | 1240 | SHYEKLKgSPEDnEQKOL--FVEQHKHYLDEIIEQISEFSKRVILAD--ANLDKVL-S AYN-KH---RDKPIREq- | 1305 |
| WP_011285506 | 1240 | SHYEKLKgSPEDnEQKOL--FVEQHKHYLDEIIEQISEFSKRVILAD--ANLDKVL-S AYN-KH---RDKPIREq- | 1305 |
| WP_011527619 | 1240 | SHYEKLKgSPEDNEQKOL--FVEQHKHYLDEIIEQISEFSKRVILAD--ANLDKVL-S AYN-KH---RDKPIREq- | 1305 |
| WP_012560673 | 1240 | SHYEKLKgSPEDnEQKOL--FVEQHKHYLDEIIEQISEFSKRVILAD--ANLDKVL-S AYN-KH---RDKPIREq- | 1305 |
| WP_014407541 | 1239 | SHYEKLKgSPEDNEQKOL--FVEQHKHYLDEIIEQISEFSKRVILAD--ANLDKVL-S AYN-KH---RDKPIREq- | 1304 |
| WP_020905136 | 1240 | SHYEKLKgSPEDnEQKOL--FVEQHKHYLDEIIEQISEFSKRVILAD--ANLDKVL-S AYN-KH---RDKPIREq- | 1305 |
| WP_023080005 | 1239 | SHYEKLKgSPEDnEQKOL--FVEQHKHYLDEIIEQISEFSKRVILAD--ANLDKVL-S AYN-KH---RDKPIREq- | 1304 |
| WP_023610282 | 1239 | SHYEKLKgSPEDnEQKOL--FVEQHKHYLDEIIEQISEFSKRVILAD--ANLDKVL-S AYN-KH---RDKPIREq- | 1304 |
| WP_030125963 | 1240 | SHYEKLKgSPEDnEQKOL--FVEQHKHYLDEIIEQISEFSKRVILAD--ANLDKVL-S AYN-KH---RDKPIREq- | 1305 |
| WP_030126706 | 1240 | SHYEKLKgSPEDnEQKOL--FVEQHKHYLDEIIEQISEFSKRVILAD--ANLDKVL-S AYN-KH---RDKPIREq- | 1305 |
| WP_031488318 | 1240 | SHYEKLKgSPEDnEQKQL--FVEQHKHYLDEIIEQISEFSKRVILAD--ANLDKVL-S AYN-KH---RDKPIREq- | 1305 |
| WP_032460140 | 1240 | SHYEKLKgSPEDnEQKOL--FVEQHKHYLDEIIEQISEFSKRVILAD--ANLDKVL-S AYN-KH---RDKPIREq- | 1305 |
| WP_032461047 | 1240 | SHYEKLKgSPEDnEQKOL--FVEQHKHYLDEIIEQISEFSKRVILAD--ANLDKVL-S AYN-KH---RDKPIREq- | 1305 |
| WP_032462016 | 1240 | SHYEKLKgSPEDnEQKOL--FVEQHKHYLDEIIEQISEFSKRVILAD--ANLDKVL-S AYN-KH---RDKPIREq- | 1305 |
| WP_032462936 | 1240 | SHYEKLKgSPEDnEQKOL--FVEQHKHYLDEIIEQISEFSKRVILAD--ANLDKVL-S AYN-KH---RDKPIREq- | 1305 |
| WP_032464890 | 1240 | SHYEKLKgSPEDnEQKOL--FVEQHKHYLDEIIEQISEFSKRVILAD--ANLDKVL-S AYN-KH---RDKPIREq- | 1305 |
| WP_033888930 | 1065 | SHYEKLKgSPEDnEQKOL--FVEQHKHYLDEIIEQISEFSKRVILAD--ANLDKVL-S AYN-KH---RDKPIREq- | 1130 |
| WP_038431314 | 1240 | SHYEKLKgSPEDnEQKOL--FVEQHKHYLDEIIEQISEFSKRVILAD--ANLDKVL-S AYN-KH---RDKPIREq- | 1305 |
| WP_038432938 | 1239 | SHYEKLKgSPEDnEQKOL--FVEQHKHYLDEIIEQISEFSKRVILAD--ANLDKVL-S AYN-KH---RDKPIREq- | 1304 |
| WP_038434062 | 1240 | SHYEKLKgSPEDNEQKQL--FVEQHKHYLDEIIEQISEFSKRVILAD--ANLDKVL-S AYN-KH---RDKPIREq- | 1305 |
| BAQ51233 | 1151 | SHYEKLKgSPEDnEQKOL--FVEQHKHYLDEIIEQISEFSKRVILAD--ANLDKVL-S AYN-KH---RDKPIREq- | 1216 |
| KGE60162 | 415 | SHYEKLKgSPEDnEQKOL--FVEQHKHYLDEIIEQISEFSKRVILAD--ANLDKVL-S AYN-KH---RDKPIREq- | 480 |
| KGE60856 | 178 | SHYEKLKgSPEDnEQKOL--FVEQHKHYLDEIIEQISEFSKRVILAD--ANLDKVL-S AYN-KH---RDKPIREq- | 243 |
| WP_002989955 | 1240 | SHYEKLKgSPEDNEQKOL--FVEQHKHYLDEIIEQISEFSKRVILAD--ANLDKVL-S AYN-KH---RDKPIREq- | 1305 |
| WP_003030002 | 1222 | YQIE----KNYE-PEHRE--YVEKHKDEFKELLEYISVFSRKYVLAD--NNLTKIE-M LFS-KN---KDAEVSS-- | 1281 |
| WP_003065552 | 1250 | QRIN----SENS-TKYLD--YVSAHKKEFEKVLSCVEDFANLYVDVE--KNLSKIR-A VAD-SM---DNFSIEE-- | 1309 |
| WP_001040076 | 1241 | SRYNESKgKPEEiEKKQE--FVNQHVSYFDDIFQIINDFSKRVILAD--ANLEKIN-R LYQ-DNk--ENIPVDE-- | 1306 |
| WP_001040078 | 1249 | SRYNESKgKPEEiEKKQE--FVNQHVSYFDDILQLINDFSKRVILAD--ANLEKIN-K LYQ-DNk--ENISVDE-- | 1314 |
| WP_001040080 | 1241 | SRYNESKgKPEEiEKKQE--FVNQHVSYFDDILOLINDFSKRVILAD--ANLEKIN-K LYQ-DNk--ENISVDE-- | 1306 |
| WP_001040081 | 1241 | SRYNESKgKPEEiEKKQE--FVNQHVSYFDDILOLINDFSKRVILAD--ANLEKIN-K LYQ-DNk--ENISVDE-- | 1306 |
| WP_001040083 | 1241 | SRYNESKgKPEEiEKKQE--FVNQHVSYFDDILQLINDFSKRVILAD--ANLEKIN-K LYQ-DNk--ENISVDE-- | 1306 |
| WP_001040085 | 1241 | SRYNESKgKPEEiEKKQE--FVNQHVSYFDDILQLINDESKRVILAD--ANLEKIN-K LYQ-DNk--ENISVDE-- | 1306 |
| WP_001040087 | 1241 | SRYNESKgKPEEiEKKQE--FVNQHVSYFDDILQLINDFSKRVILAD--ANLEKIN-K LYQ-DNk--ENISVDE-- | 1306 |
| WP_001040088 | 1241 | SRYNESKgKPEEiEKKQE--FVNQHVSYFDDILQLINDESKRVILAD--ANLEKIN-K LYQ-DNk--ENISVDE-- | 1306 |
| WP_001040089 | 1241 | SRYNESKgKPEEiEKKQE--FVNQHVSYFDDILOLINDFSKRVILAD--ANLEKIN-K LYQ-DNk--ENISVDE-- | 1306 |
| WP_001040090 | 1241 | SRYNESKgKPEEiEKKQE--FVNQHVSYFDDILQLINDESKRVILAD--ANLEKIN-K LYQ-DNk--ENISVDE-- | 1306 |
| WP_001040091 | 1241 | SRYNESKgKPEEiEKKQE--FVNQHVSYFDDILQLINDESKRVILAD--ANLEKIN-K LYQ-DNk--ENISVDE-- | 1306 |
| WP_001040092 | 1241 | SRYNESKgKPEEiEKKQE--FVNQHISYFDDILOLINDFSKRVILAD--ANLEKIN-K LYS-DNk--DNTPVDE-- | 1306 |
| WP_001040094 | 1241 | SRYNELKgKPEEiEQKQE--FVVQHVSYFDDILQIINDFSNRVILAD--ANLEKIN-K LYQ-DNk--ENIPVDE-- | 1306 |
| WP_001040095 | 1241 | SRYNELKgKPEEiEQKQE--FVVQHVSYFDDILQIINDFSNRVILAD--ANLEKIN-K LYQ-DNk--ENIPVDE-- | 1306 |
| WP_001040096 | 1241 | SRYNELKgKPEEiEQKQE--FVVQHVSYFDDILQIINDFSNRVILAD--ANLEKIN-K LYQ-DNk--ENIPVDE-- | 1306 |
| WP_001040097 | 1241 | SRYNELKgKPEEiEQKQE--FVVQHVSYFDDILQIINDFSNRVILAD--ANLEKIN-K LYQ-DNk--ENIPVDE-- | 1306 |
| WP_001040098 | 1241 | SRYNELKgKPEEiEQKQE--FVVQHVSYFDDILQIINDFSNRVILAD--ANLEKIN-K LYQ-DNk--ENIPVDE-- | 1306 |
| WP_001040099 | 1241 | SRYNELKgKPEEiEQKQE--FVVQHVSYFDDILQIINDFSNRVILAD--ANLEKIN-K LYQ-DNk--ENIPVDE-- | 1306 |
| WP_001040100 | 1241 | SRYNELKgKPEEEiQKQE--FVVQHVSYFDDILQIINDFSNRVILAD--ANLEKIN-K LYQ-DNk--ENIPVDE-- | 1306 |
| WP_001040104 | 1241 | SRYNESKgKPEEiEKKQE--FVNQHVSYFDDILQLINDESKRVILAD--ANLEKIN-K LYQ-DNk--ENISVDE-- | 1306 |
| WP_001040105 | 1241 | SRYNESKgKPEEiEKKQE--FVNQHVSYFDDILQLINDFSKRVILAD--ANLEKIN-K LYQ-DNk--ENISVDE-- | 1306 |
| WP_001040106 | 1241 | SRYNELKgKPEEiEQKQE--FVVQHVSYFDDILQIINDFSNRVILAD--ANLEKIN-K LYQ-DNk--ENISVDE-- | 1306 |
| WP_001040107 | 1241 | SRYNELKgKPEEiEQKQE--FVVQHVSYFDDILQIINDFSNRVILAD--ANLEKIN-K LYQ-DNk--ENISVDE-- | 1306 |
| WP_001040108 | 1241 | SRYNELKgKPEEiEQKQE--FVVQHVSYFDDILQIINDFSNRVILAD--ANLEKIN-K LYQ-DNk--ENISVDE-- | 1306 |
| WP_001040109 | 1241 | SRYNELKgKPEEiEQKQE--FVVQHVSYFDDILQIINDFSNRVILAD--ANLEKIN-K LYQ-DNk--ENISVDE-- | 1306 |
| WP_001040110 | 1241 | SRYNELKgKPEEiEQKQE--FVVQHVSYEDDILQIINDFSNRVILAD--ANLEKIN-K LYQ-DNk--ENISVDE-- | 1306 |
| WP_015058523 | 1241 | SRYNESKgKPEEiEKKQE--FVNQHVSYFDDILOLINDFSKRVILAD--ANLEKIN-K LYS-DNk--DNTPVDE-- | 1306 |
| WP_017643650 | 1241 | SRYNELKgKPEEiEQKQE--FVVQHVSYFDDILQIINDFSNRVILAD--ANLEKIN-K LYQ-DNk--ENIPVDE-- | 1306 |
| WP_017647151 | 1241 | SRYNELKgKPEEiEQKQE--FVVQHVSYFDDILQIINDFSNRVILAD--ANLEKIN-K LYQ-DNk--ENISVDE-- | 1306 |
| WP_017648376 | 1241 | SRYNELKgKPEEiEQKQE--FVVQHVSYFDDILQIINDFSNRVILAD--ANLEKIN-K LYQ-DNk--ENISVDE-- | 1306 |
| WP_017649527 | 1241 | SRYNESKgKPEEiEKKQE--FVNQHVSYFDDILOLINDFSKRVILAD--ANLEKIN-K LYQ-DNk--ENISVDE-- | 1306 |
| WP_017771611 | 1241 | SRYNELKgKPEEiEQKQE--FVVOHVSYFDDILQIINDFSNRVILAD--ANLEKIN-K LYQ-DNk--ENISVDE-- | 1306 |
| WP_017771984 | 1241 | SRYNESKgKPEEiEKKQE--FVNQHVSYFDDILQLINDESKRVILAD--ANLEKIN-K LYQ-DNk--ENISVDE-- | 1306 |
| CFQ25032 | 1241 | SRYNESKgKPEEiEKKQE--FVNQHVSYFDDILOLINDFSKRVILAD--ANLEKIN-K LYQ-DNk--ENISVDE-- | 1306 |
| CFV16040 | 1241 | SRYNESKgKPEEiEKKQE--FVNQHVSYFDDILOLINDFSKRVILAD--ANLEKIN-K LYQ-DNk--ENISVDE-- | 1306 |
| KLJ37842 | 1241 | SRYNESKgKPEEiEKKQE--FVNQHVSYFDDILQLINDFSKRVILAD--ANLEKIN-K LYQ-DNk--ENISVDE-- | 1306 |
| KLJ72361 | 1241 | SRYNESKgKPEEiEKKQE--FVNQHVSYFDDILQLINDFSKRVILAD--ANLEKIN-K LYQ-DNk--ENISVDE-- | 1306 |
| KLL20707 | 1255 | SRYNESKgKPEEiEKKQE--FVNQHVSYFDDILQLINDFSKRVILAD--ANLEKIN-K LYQ-DNk--ENISVDE-- | 1320 |
| KLL42645 | 1241 | SRYNELKgKPEEiEQKQE--FVVQHVSYFDDILQIINDFSNRVILAD--ANLEKIN-K LYQ-DNk--ENISVDE-- | 1306 |
| WP_047207273 | 1241 | SRYNESKgKPEEiEKKQE--FVNQHVSYFDDILQLINDESKRVILAD--ANLEKIN-K LYQ-DNk--ENISVDE-- | 1306 |
| WP_047209694 | 1241 | SRYNELKgKPEEiEQKQE--FVVQHVSYFDDILQIINDFSNRVILAD--ANLEKIN-K LYQ-DNk--ENIPVDE-- | 1306 |
| WP_050198062 | 1241 | SRYNESKgKPEEiEKKQE--FVNQHVSYFDDILQLINDFSKRVILAD--ANLEKIN-K LYQ-DNk--ENISVDE-- | 1306 |
| WP_050201642 | 1241 | SRYNESKgKPEEiEKKQE--FVNQHVSYFDDILOLINDFSKRVILAD--ANLEKIN-K LYQ-DNk--ENISVDE-- | 1306 |
| WP_050204027 | 1241 | SRYNELKgKPEEiEQKQE--FVVQHVSYFDDILQIINDFSNRVILAD--ANLEKIN-K LYQ-DNk--ENISVDE-- | 1306 |
| WP_050881965 | 1241 | SRYNESKgKPEEiEKKQE--FVNQHVSYFDDILOLINDFSKRVILAD--ANLEKIN-K LYQ-DNk--ENISVDE-- | 1306 |
| WP_050886065 | 1241 | SRYNESKgKPEEiEKKQE--FVNQHVSYFDDILQLINDFSKRVILAD--ANLEKIN-K LYQ-DNk--ENISVDE-- | 1306 |
| AHN30376 | 1241 | SRYNESKgKPEEiEKKQE--FVNQHVSYFDDILQLINDESKRVILAD--ANLEKIN-K LYS-DNk--DNTPVDE-- | 1306 |
| EAO78426 | 1241 | SRYNESKgKPEEiEKKQE--FVNQHVSYFDDILQLINDFSKRVILAD--ANLEKIN-K LYQ-DNk--ENISVDE-- | 1306 |
| CCW42055 | 1241 | SRYNESKgKPEEiEKKQE--FVNQHVSYFDDILQIINDFSNRVILAD--ANLEKIN-K LYQ-DNk--ENISVDE-- | 1306 |
| WP_003041502 | 1254 | KRIN----NPIN-KDHIE--YVKKHRDDFKELLNYVLEFNEKYVGAT--KNGERLK-E AVA-DF---DSKSNEE-- | 1313 |
| WP_037593752 | 1223 | YQIE----KNYE-PEHRE--YVEKHKDEFKELLEYISVFSRKYVLAD--NNLTKIE-M LFS-KN---KDAEVSS-- | 1282 |
| WP_049516684 | 1223 | YRIE----KDYE-PEHRE--YVEKHKDEFKELLEYISVFSRKYVLAD--NNLTKIE-M LFS-KN---KDAEVSS-- | 1282 |
| GAD46167 | 1222 | YQIE----KNYE-PEHRE--YVEKHKDEFKELLEYISVFSRKYVLAD--NNLTKIE-M LFS-KN---KDAEVSS-- | 1281 |
| WP_018363470 | 1254 | HRIG----NFNS-AEHLK--YVSEHKKEFEEVLSCVENFANVYVDVE--KNLSKIR-A AAD-SM---DNFSIEE-- | 1313 |
| WP_003043819 | 1249 | QNISATTgSNNLg-------YIEQHREEFKEIFEKIIDFSEKYILKN--KVNSNLK-S SFD-EQfavSDSIL--1- | 1310 |
| WP_006269658 | 1222 | YRIE----KDYE-PEHRE--YVEKHKDEFKELLEYISVFSRKYVLAD--NNLTKIE-M LFS-KN---KDAEVSS-- | 1281 |
| WP_048800889 | 1242 | HRID----NSDN-SEHLK--YITEHKEEFGKLLSYIENFAKSYVDVD--KNLEKIQ-L AVE-KI---DSFSVKE-- | 1301 |
| WP_012767106 | 1246 | -HAHKIESSKE--LEHEA--YILDHYNDLYQLLSYIERFASLYVDVE--KNISKVK-E LFS-NI---ESYSISEi- | 1308 |
| WP_014612333 | 1246 | -HAHKIESSKE--LEHEA--YILDHYNDLYQLLSYIERFASLYVDVE--KNISKVK-E LFS-NI---ESYSISEi- | 1308 |
| WP_015017095 | 1246 | -HAHKIESSKE--LEHEA--YILDHYNDLYQLLSYIERFASLYVDVE--KNISKVK-E LFS-NI---ESYSISEi- | 1308 |
| WP_015057649 | 1246 | -HAHKIESSKE--LEHEA--YILDHYNDLYQLLSYIERFASLYVDVE--KNISKVK-E LFS-NI---ESYSISEi- | 1308 |
| WP_048327215 | 1246 | -HAHKIESSKE--LEHEA--YILDHYNDLYQLLSYIERFASLYVDVE--KNISKVK-E LFS-NI---ESYSISEi- | 1308 |
| WP_049519324 | 1246 | -HAHKIESSKE--LEHEA--YILDHYNDLYQLLSYIERFASLYVDVE--KNISKVK-E LFS-NI---ESYSISEI- | 1308 |
| WP_012515931 | 1220 | AHYHELTgSSEDVLRKKY--FVDRHLHYFDDIIQMINDFAERHILAS--SNLEKIN-H TYH-NN---SDLPVNEr- | 1285 |
| WP_021320964 | 1220 | AHYHELTgSSEDVLRKKY--FVERHLHYFDDIIQMINDFAERHILAS--SNLEKIN-H TYH-NN---SDLPINEr- | 1285 |
| WP_037581760 | 1220 | AHYHELTgSSEDVLRKKY--FVERHLHYFDDIIQMINDFAERHILAS--SNLEKIN-H TYH-NN---SDLPVNEr- | 1285 |
| WP_004232481 | 1252 | QHVN----NSHK-PEHLN--YVKQHKDEFKDIFNLIISIARINILKP--KVVDNL--- -IN-EF---TEYGQED-- | 1308 |
| WP_009854540 | 1247 | HRAD----NFNS-TEYLN--YVSEHKKEFEKVLSCVEDFANLYVDVE--KNLSKIR-A VAD-SM---DNFSIEE-- | 1306 |
| WP_012962174 | 1248 | HRVN----SFNN-SEHLK--YVSEHKKEFGEVLSCVENFAKSYVDVE--KNLGKIR-A VAD-KI---DTFSIED-- | 1307 |
| WP_039695303 | 1249 | HRAD----NENS-TEYLN--YVSEHKKEFEKVLSCVEDFANLYVDVE--KNLSKIR-A VAD-SM---DNFSIEE-- | 1308 |
| WP_014334983 | 1252 | HRID----SFNS-TEHLK--YVSEHKKEFEKVLSCVENFSNLYVDVE--KNLSKVR-A AAE-SM---TNFSLEE-- | 1311 |
| WP_003099269 | 1240 | SHYTKFTgKEEDrEKKRS--YVESHLYYFDEIMQIIVEYSNRYILAD--SNLIKIQ-N LYK-EKd---NFSIEEq- | 1305 |
| AHY15608 | 1240 | SHYTKFTgKEEDrEKKRS--YVESHLYYFXEVKSSF---------------------- ------------------ | 1273 |
| AHY17476 | 1240 | SHYTKFTgKEEDrEKKRS--YVESHLYXFX---------------------------- ------------------ | 1267 |
| ESR09100 | 72 | SHYTKFTgKEEDrEKKRS--YVESHLYYFDEIMQIIVEYSNRYILAD--SNLIKIQ-N LYK--Ek--DNFSIEEq- | 137 |
| AGM98575 | 1240 | SHYTKFTgKEEDrEKKRS--YVESHLYYFDVRLSQVERVINVEF-------------- ------------------ | 1281 |
| ALF27331 | 1222 | KNIH----KVDE-PKHLD--YVKKHKDEFKELLDVVSNFSKKNILAE--SNLEKIE-E LYA-QN---NNKDITE-- | 1281 |
| WP_018372492 | 1229 | KKVDVLVKSKDD---DYD---LEEHRAEFAELLDCIKKENDMYILAS--SNMSKIE-E IYQ-KNi---DAPIEE-- | 1289 |
| WP_045618028 | 1252 | QRIN----KISE-PIHKQ--YVETHQSEFKELLTAIISLSKKYI-QK--PNVESL--- LQQ-AF---DQSDKDIyq | 1310 |
| WP_045635197 | 1247 | KNVH----KLDE-PGHLE--YIQKHRNEFKDLLNLVSEFSQKYVLAD--ANLEKIK-S LYA-DN---EQADIEI-- | 1306 |
| WP_002263549 | 1222 | KNIH----KVDE-PKHLD--YVDKHKDEFKELLDVVSNFSKKYTLAE--GNLEKIK-E LYA-QN---NGEDLKE-- | 1281 |
| WP_002263887 | 1222 | KNIH----KVDE-PKHLD--YVDKHKDEFKELLDVVSNFSKKYTLAE--GNLEKIK-E LYA-QN---NGEDLKE-- | 1281 |
| WP_002264920 | 1222 | KNIH----KVDE-PKHLD--YVDKHKDEFKELLDVVSNFSKKYTLAE--GNLEKIK-E LYA-QN---NGEDLKE-- | 1281 |
| WP_002269043 | 1222 | KNIH----KVDE-PKHLD--YVDKHKDEFKELLDVVSNFSKKYTLAE--GNLEKIK-E LYA-QN---NGEDLKE-- | 1281 |
| WP_002269448 | 1222 | KNIH----KVDE-PKHLD--YVDKHKDEFKELLDVVSNFSKKYTLAE--GNLEKIK-E LYA-QN---NGEDLKE-- | 1281 |
| WP_002271977 | 1222 | KNIH----KVDE-PKHLD--YVDKHKDEFKELLDVVSNFSKKYTLAE--GNLEKIK-E LYA-QN---NGEDLKE-- | 1281 |
| WP_002272766 | 1222 | KNIH----KVDE-PKHLD--YVDKHKDEFKELLDVVSNFSKKYTLAE--GNLEKIK-E LYA-QN---NGEDLKE-- | 1281 |
| WP_002273241 | 1222 | KNIH----KVDE-PKHLD--YVDKHKDEFKELLDVVSNFSKKYTLAE--GNLEKIK-E LYA-QN---NGEDLKE-- | 1281 |
| WP_002275430 | 1222 | KNIH----KVDE-PKHLD--YVDKHKDEFKELLDVVSNFSKKYTLAE--GNLEKIK-E LYA-QN---NGEDLKE-- | 1281 |
| WP_002276448 | 1222 | KNIH----KVDE-PKHLD--YVDKHKDEFKELLDVVSNFSKKYTLAE--GNLEKIK-E LYA-QN---NGEDLKE-- | 1281 |
| WP_002277050 | 1230 | HHL-------DN-DYSNE--YVKNHYQQFDILFNEITSFSKKCKLGK--EHIQKIE-E AYSKER---DSASIEE-- | 1287 |
| WP_002277364 | 1222 | KNIH----KVDE-PKHLD--YVDKHKDEFKELLDVVSNFSKKYTLAE--GNLEKIK-E LYA-QN---NGEDLKE-- | 1281 |
| WP_002279025 | 1222 | KNIH----KVDE-PKHLD--YVDKHKDEFKELLDVVSNFSKKYTLAE--GNLEKIK-E LYA-ON---NGEDLKE-- | 1281 |
| WP_002279859 | 1222 | KNIH----KVDE-PKHLD--YVDKHKDEFKELLDVVSNFSKKYTLAE--GNLEKIK-E LYA-QN---NGEDLKE-- | 1281 |
| WP_002280230 | 1222 | KNIH----KVDE-PKHLD--YVDKHKDEFKELLDVVSNFSKKYTLAE--GNLEKIK-E LYA-QN---NGEDLKE-- | 1281 |
| WP_002281696 | 1222 | KNIH----KVDE-PKHLD--YVDKHKDEFKELLDVVSNFSKKYTLAE--GNLEKIK-E LYA-QN---NGEDLKE-- | 1281 |
| WP_002282247 | 1230 | HHL-------DN-DYSNE--YVKNHYQQFDILFNEITSFSKKCKLGK--EHIQKIE-E AYSKER---DFASIEE-- | 1287 |
| WP_002282906 | 1222 | KNIH----KVDE-PKHLD--YVDKHKDEFKELLDVVSNFSKKYTLAE--GNLEKIK-E LYA-QN---NGEDLKE-- | 1281 |
| WP_002283846 | 1222 | KNIH----KVDE-PKHLD--YVDKHKDEFKELLDVVSNFSKKYTLAE--GNLEKIK-E LYA-QN---NGEDLKE-- | 1281 |
| WP_002287255 | 1222 | KNIH----KVDE-PKHLD--YVDKHKDEFKELLDVVSNFSKKYTLAE--GNLEKIK-E LYA-QN---NGEDLKE-- | 1281 |
| WP_002288990 | 1222 | KNIH----KVDE-PKHLD--YVDKHKDEFKELLDVVSNFSKKYTLAE--GNLEKIK-E LYA-QN---NGEDLKE-- | 1281 |
| WP_002289641 | 1222 | KNIH----KVDE-PKHLD--YVDKHKDEFKELLDVVSNFSKKYTLAE--GNLEKIK-E LYA-QN---NGEDLKE-- | 1281 |
| WP_002290427 | 1222 | KNIH----KVDE-PKHLD--YVDKHKDEFKELLDVVSNFSKKYTLAE--GNLEKIK-E LYA-QN---NGEDLKE-- | 1281 |
| WP_002295753 | 1222 | KNIH----KVDE-PKHLD--YVDKHKDEFKELLDVVSNFSKKYTLAE--GNLEKIK-E LYA-QN---NGEDLKE-- | 1281 |
| WP_002296423 | 1222 | KNIH----KVDE-PKHLD--YVDKHKDEFKELLDVVSNFSKKYTLAE--GNLEKIK-E LYA-QN---NGEDLKE-- | 1281 |
| WP_002304487 | 1236 | KNIH----KVDE-PKHLD--YVDKHKDEFKELLDVVSNFSKKYTLAE--GNLEKIK-E LYA-ON---NGEDLKE-- | 1295 |
| WP_002305844 | 1222 | KNIH----KVDE-PKHLD--YVDKHKDEFKELLDVVSNFSKKYTLAE--GNLEKIK-E LYA-QN---NGEDLKE-- | 1281 |
| WP_002307203 | 1222 | KNIH----KVDE-PKHLD--YVDKHKDEFKELLDVVSNFSKKYTLAE--GNLEKIK-E LYA-QN---NGEDLKE-- | 1281 |
| WP_002310390 | 1222 | KNIH----KVDE-PKHLD--YVDKHKDEFKELLDVVSNFSKKYTLAE--GNLEKIK-E LYA-QN---NGEDLKE-- | 1281 |
| WP_002352408 | 1222 | KNIH----KVDE-PKHLD--YVDKHKDEFKELLDVVSNFSKKYTLAE--GNLEKIK-E LYA-QN---NGEDLKE-- | 1281 |
| WP_012997688 | 1222 | KNIH----KVDE-PKHLD--YVDKHKDEFKELLDVVSNFSKKYTLAE--GNLEKIK-E LYA-QN---NGEDLKE-- | 1281 |
| WP_014677909 | 1222 | KNIH----KVDE-PKHLD--YVDKHKDEFKELLDVVSNFSKKYTLAE--GNLEKIK-E LYA-QN---NGEDLKE-- | 1281 |
| WP_019312892 | 1222 | KNIH----KVDE-PKHLD--YVDKHKDEFKELLDVVSNFSKKYTLAE--GNLEKIK-E LYA-QN---NGEDLKE-- | 1281 |
| WP_019313659 | 1222 | KNIH----KVDE-PKHLD--YVDKHKDEFKELLDVVSNFSKKYTLAE--GNLEKIK-E LYA-QN---NGEDLKE-- | 1281 |
| WP_019314093 | 1222 | KNIH----KVDE-PKHLD--YVDKHKDEFKELLDVVSNFSKKYTLAE--GNLEKIK-E LYA-QN---NGEDLKE-- | 1281 |
| WP_019315370 | 1222 | KNIH----KVDE-PKHLD--YVDKHKDEFKELLDVVSNFSKKYTLAE--GNLEKIK-E LYA-QN---NGEDLKE-- | 1281 |
| WP_019803776 | 1222 | KNIH----KVDE-PKHLD--YVDKHKDEFKELLDVVSNFSKKYTLAE--GNLEKIK-E LYA-QN---NGEDLKE-- | 1281 |
| WP_019805234 | 1222 | KNIH----KVDE-PKHLD--YVDKHKDEFKELLDVVSNFSKKYTLAE--GNLEKIK-E LYA-QN---NGEDLKE-- | 1281 |
| WP_024783594 | 1222 | KNIH----KVDE-PKHLD--YVDKHKDEFKELLDVVSNFSKKYTLAE--GNLEKIK-E LYA-QN---NGEDLKE-- | 1281 |
| WP_024784288 | 1230 | HHL-------DN-DYSNE--YVKNHYQQFDILFNEITSFSKKCKLGK--EHIQKIE-E AYSKER---DFASIEE-- | 1287 |
| WP_024784666 | 1222 | KNIH----KVDE-PKHLD--YVDKHKDEFKELLDVVSNFSKKYTLAE--GNLEKIK-E LYA-QN---NGEDLKE-- | 1281 |
| WP_024784894 | 1222 | KNIH----KVDE-PKHLD--YVDKHKDEFKELLDVVSNFSKKYTLAE--GNLEKIK-E LYA-QN---NGEDLKE-- | 1281 |
| WP_024786433 | 1230 | HHL-------DN-DYSNE--YVKNHYQQFDILFNEITSFSKKCKLGK--EHIQKIE-E AYSKER---DSASIEE-- | 1287 |
| WP_049473442 | 1222 | KNIH----KVDE-PKHLD--YVDKHKDEFKELLDVVSNFSKKYTLAE--GNLEKIK-E LYA-QN---NGEDLKE-- | 1281 |
| WP_049474547 | 1222 | KNIH----KVDE-PKHLD--YVDKHKDEFKELLDVVSNFSKKYTLAE--GNLEKIK-E LYA-QN---NGEDLKE-- | 1281 |
| EMC03581 | 1215 | KNIH----KVDE-PKHLD--YVDKHKDEFKELLDVVSNFSKKYTLAE--GNLEKIK-E LYA-QN---NGEDLKE-- | 1274 |
| WP_000428612 | 1250 | KNIH----RLDE-PEHLE--YIQKHRNEFKGLLNLVSEFSQKYVLAD--ANLEKIK-N LYA-DN---EQADIEI-- | 1309 |
| WP_000428613 | 1248 | KNVH----KLDE-PEHLE--YIQKHRNEFKDLLNLVSEFSQKYVLAD--ANLEKIQ-N LYA-DN---EQADIEI-- | 1307 |
| WP_049523028 | 1243 | KRIQ----KKDE-PEHLE--YIKQHHSEFNDLLNFVSEFSQKYVLAE--SNLEKIK-N LYI-DN---EQTNMEE-- | 1302 |
| WP_003107102 | 1209 | SRYTSFSgKEEDrEKHRH--FVESHLHYFDEIKDIIADFSRRYILAD--ANLEKIL-T LYN-EKn---QFSIEEq- | 1274 |
| WP_054279288 | 1242 | SKRDK--gTQSEnME-----YISNHKEKFIEIFHYIIRYAEKNVIKP--KVIERLN-D TFNqKF---NDSDLTE1- | 1303 |
| WP_049531101 | 1252 | QRIN----KISE-PIHKQ--YVETHOSEFEELLTTIISLSKKYI-QK--PIVESL--- LQQ-AF---EQADKDIyq | 1310 |
| WP_049538452 | 1252 | QRIN----KISE-PIHKQ--YVEAHQNEFKELLTTIISLSKKYI-QK--PNVESL--- LQQ-AF---EQADKDIyq | 1310 |
| WP_049549711 | 1254 | QRIN----KFSE-PIHKQ--YVEAHQNEFKELLTIIISLSKKYI-QK--PNVESL--- LHQ-AF---EQADNDIyq | 1312 |
| WP_007896501 | 1246 | SRYDKLSSKIESeQQKKL--FVEQHLHYFDEILDIVVKHATCYIKAE--NNLKKII-S LYK-KK---EAYSINEq- | 1311 |
| EFR44625 | 1198 | SRYDKLSSKIESeQQKKL--FVEQHLHYFDEILDIVVKHATCYIKAE--NNLKKII-S LYK-KK---EAYSINEq- | 1263 |
| WP_002897477 | 1247 | KNLH----KLDE-PEHLE--YIQKHRNEFKDLLNLVSEFSQKYILAE--ANLEKIK-D LYA-DN---EQADIEI-- | 1306 |
| WP_002906454 | 1253 | QRIN----KISE-PIHKQ--YVEAHQNEFKELLTTIISLSKKYI-QK--PNVELL--- LQQ-AF---DQADKDIyq | 1311 |
| WP_009729476 | 1248 | KNVH----KLDE-PGHLE--YIQKHRNEFKDLLNLVSEFSQKYVLAD--ANLEKIK-N LYA-DN---EQADIEI-- | 1307 |
| CQR24647 | 1238 | QHAN----KEDS----VI--YLEKHRHELSELFHHIIGVSEKTILKP--KVEMTLN-E AFE-KHf--EFDEVSE-- | 1295 |
| WP_000066813 | 1252 | KNVH----KLDE-PEHLE--YIQKHRYEFKDLLNLVSEFSQKYVLAD--ANLEKIK-N LYA-DN---EQADIEI-- | 1311 |
| WP_009754323 | 1248 | KNVH----KLDE-PEHLE--YIQKHRYEFKDLLNLVSEFSQKYVLAE--ANLEKIK-S LYV-DN---EQADIEI-- | 1307 |
| WP_044674937 | 1241 | SNIH----KITE-PIHLN--YVNKNKHEFKELLRHISDFSTRYILAQ--DRLSKIE-E LYD-KN---DGDDISD-- | 1300 |
| WP_044676715 | 1243 | SNIH----KITE-PIHLN--YVNKNKHEFKELLRHISDFSTRYILAQ--DRLSKIE-E LYD-KN---DGDDISD-- | 1302 |
| WP_044680361 | 1243 | SNIH----KITE-PIHLN--YVNKNKHEFKELLRHISDFSTRYILAQ--DRLSKIE-E LYD-KN---DGDDISD-- | 1302 |
| WP_044681799 | 1241 | SNIH----KITE-PIHLN--YVNKNKHEFKELLRHISDFSTRYILAQ--DRLSKIE-E LYD-KN---DGDDISD-- | 1300 |
| WP_049533112 | 1254 | KRIN----NPIN-KDHIE--YVKKHRDDFKELLNYVLEFNEKYVGAT--KNGERLK-E AVA-DF---DSKSNEE-- | 1313 |
| WP_029090905 | 1182 | STKQA-----DE-AMFLKyyRLEHLEAVFEEL---IRKQAADYQIFE--KLIKKIEvN FYS----c----TYNEk- | 1240 |
| WP_006506696 | 1212 | YNAIYKQ-DYDNIDDILMi-----------QLYIELTNKMKVLYPAY-rGIAEKFE-S YVV----i----SKEEk- | 1268 |
| AIT42264 | 1240 | SHYEKLKgSPEDnEQKOL--FVEQHKHYLDEIIEQISEFSKRVILAD--ANLDKVL-S AYN-KH---RDKPIREq- | 1305 |
| WP_034440723 | 1218 | KHYNE-----DE--TSHK--FIVEHKAYFDELLNYIVEFANKYLELE--NSIEKIK-D LYH-----gKGPDVEEke | 1276 |
| AKQ21048 | 1240 | SHYEKLKgSPEDnEQKOL--FVEQHKHYLDEIIEQISEFSKRVILAD--ANLDKVL-S AYN-KH---RDKPIREq- | 1305 |
| WP_004636532 | 1212 | NRYDKVK-----fPDSIE--YVHDNLAKFDDLLEYVIDESNKYINAD--KNVQKIQ-K IYK-EH---GTEDVEL-- | 1271 |
| WP_002364836 | 1218 | KQCLL----PNQ-SESLA--YVEQHQPEFQEILERVVDFAEVHTLAK--SKVQQIV-K LFE-AN---QTADVKE-- | 1277 |
| WP_016631044 | 1169 | KQCLL----PNQ-SESLA--YVEQHOPEFQEILERVVDFAEVHTLAK--SKVQQIV-K LFE-AN---QTADVKE-- | 1228 |
| EMS75795 | 954 | QHYDEIAhKESF-----D--YVNDHLSEFREILDQVIDFSNRYTIAA--KNTEKIA-E LFE-QN---QESTVQS-- | 1013 |
| WP_002373311 | 1218 | KOCLL----PNQ-SESLA--YVEQHQPEFQEILERVVDFAEVHTLAK--SKVQQIV-K LFE-AN---QTADVKE-- | 1277 |
| WP_002378009 | 1218 | KQCLL----PNQ-SESLA--YVEQHOPEFQEILERVVDFAEVHTLAK--SKVQQIV-K LFE-AN---QTADVKE-- | 1277 |
| WP_002407324 | 1218 | KQCLL----PNQ-SESLA--YVEQHQPEFQEILERVVDFAEVHTLAK--SKVQQIV-K LFE-AN---QTADVKE-- | 1277 |
| WP_002413717 | 1218 | KQCLL----PNQ-SESLA--YVEQHOPEFQEILERVVDFAEVHTLAK--SKVQQIV-K LFE-AN---QTADVKE-- | 1277 |
| WP_010775580 | 1220 | KQCLL----PNQ-SESLA--YVEQHOPEFQEILERVVDFAEVHTLAK--SKVOQIV-K LFE-AN---QTADVKE-- | 1279 |
| WP_010818269 | 1218 | KQCLL----PNQ-SESLA--YVEQHOPEFQEILERVVDFAEVHTLAK--SKVQQIV-K LFE-AN---QTADVKE-- | 1277 |
| WP_010824395 | 1218 | KQCLL----PNQ-SESLA--YVEQHOPEFQEILERVVDFAEVHTLAK--SKVQQIV-K LFE-AN---QTADVKE-- | 1277 |
| WP_016622645 | 1218 | KQCLL----PNQ-SESLA--YVEQHOPEFQEILERVVDFAEVHTLAK--SKVQQIV-K LFE-AN---QTADVKE-- | 1277 |
| WP_033624816 | 1218 | KQCLL----PNQ-SESLA--YVEQHOPEFQEILERVVDFAEVHTLAK--SKVQQIV-K LFE-AN---QTADVKE-- | 1277 |
| WP_033625576 | 1218 | KQCLL----PNQ-SESLA--YVEQHOPEFQEILERVVDFAEVHTLAK--SKVQQIV-K LFE-TN---QTADVKE-- | 1277 |
| WP_033789179 | 1218 | KOCLL----PNQ-SESLA--YVEQHOPEFQEILERVVDFAEVHTLAK--SKVQQIV-K LFE-AN---QTADVKE-- | 1277 |
| WP_002310644 | 1220 | KQYDEIShKESF-----D--YVNEHHKEFSEVFARVLEFAGKYTLAE--KNIEKLE-K IYK-EN---QTDDLAK-- | 1279 |
| WP_002312694 | 1221 | KQYDEIShKESF-----D--YVNEHHKEFSEVFARVLEFAGKYTLAE--KNIEKLE-K IYK-EN---QTDDLAK-- | 1280 |
| WP_002314015 | 1221 | KQYDEIShKESF-----D--YVNEHHKEFSEVFARVLEFAGKYTLAE--KNIEKLE-K IYK-EN---QTDDLAK-- | 1280 |
| WP_002320716 | 1221 | KQYDEIShKESF-----D--YVNEHHKEFSEVFARVLEFAGKYTLAE--KNIEKLE-K IYK-EN---QTDDLAK-- | 1280 |
| WP_002330729 | 1220 | KQYDEIShKESF-----D--YVNEHHKEFSEVFARVLEFAGKYTLAE--KNIEKLE-K IYK-EN---QTDDLAK-- | 1279 |
| WP_002335161 | 1221 | KQYDEIShKESF-----D--YVNEHHKEFSEVFARVLEFAGKYTLAE--KNIEKLE-K IYK-EN---QTDDLAK-- | 1280 |
| WP_002345439 | 1221 | KQYDEIShKESF-----D--YVNEHHKEFSEVFARVLEFAGKYTLAE--KNIEKLE-K IYK-EN---QTDDLAK-- | 1280 |
| WP_034867970 | 1211 | QHYDKITyQESF-----D--YVNTHLSDFSAILTEVLAFAEKYTLAD--KNIERIQ-E LYE-EN---KYGETSM-- | 1270 |
| WP_047937432 | 1221 | KQYDEIShKESF-----D--YVNEHHKEFSEVFARVLEFAGKYTLAE--KNIEKLE-K IYK-EN---QTDDLAK-- | 1280 |
| WP_010720994 | 1211 | QHYDKITyQESF-----D--YVNTHLSDFSAILTEVLAFAEKYTLAD--KNIERIQ-E LYE-EN---KYGEISM-- | 1270 |
| WP_010737004 | 1211 | QHYDKITyQESF-----D--YVNTHLSDFSAILTEVLAFAEKYTLAD--KNIERIQ-E LYE-EN---KYGETSM-- | 1270 |
| WP_034700478 | 1211 | QHYDKITyQESF-----D--YVNTHLSDFSAILTEVLAFAEKYTLAD--KNIERIQ-E LYE-EN---KYGEISM-- | 1270 |
| WP_007209003 | 1209 | KKIIN--gKNSD---SVS--YIQNNKEKFREIFEYIVDFSSKYISAD--ANLNKIE-K IFE-NNfh----KASEqe | 1269 |
| WP_023519017 | 1205 | RHYDEINhKVSF-----D--YVNAHKEGENDIFDFISDFGVRYILAP--QHLEKIK-V AYE-KN---KEVDLKE-- | 1264 |
| WP_010770040 | 1216 | KOVDE-----DS-GKSEE--YVREHRAEFAEILNYVQAFSETKILAN--KNLQTIL-K LYE-EN---KEADIKE-- | 1274 |
| WP_048604708 | 1213 | KHCNE-----KP-D-SLK--YVTEHQSGFSEIMAHVKDFAEKYTLVD--KNLEKIL-S LYA-KN---MDSEVKE-- | 1270 |
| WP_010750235 | 1214 | NHYDEIAyKDSY-----D--YVNEHFSNFQDILDKVIIFAEKYTSAP--QKLNQII-A TYE-KN---QEADRKI-- | 1273 |
| AII16583 | 1279 | SHYEKLKgSPEDNEQKQL--FVEQHKHYLDEIIEQISEFSKRVILAD--ANLDKVL-S AYN-KH---RDKPIREq- | 1344 |
| WP_029073316 | 1226 | YKAMKYK-NYSE1SQEEIm-----------NVYDIFVEKLKLYYPTY-KNIATNFE-N FEN----1----SDEEk- | 1282 |
| WP_031589969 | 1226 | YKAMKYK-NYDNIDSEKII-----------DLYRLLINKMELYYPEYrkQLVKKFE-D LKV----i----SIEEk- | 1283 |
| KDA45870 | 1200 | NAKDG--------EQKLE-----DHKAEFKELFDKIMEFADKYVVAP--KNSEKIR-R LYE-ENq-----DATPme | 1253 |
| WP_039099354 | 1242 | LPLTQ-----SEeLAEQV----------YDEILDQVMHYFPLYDTNQfrAKLSAGKaA DGN-KMv-----QVGQqv | 1306 |
| AKP02966 | 1238 | QIPDE-----DpDQILAf----YDKNILVEILQELITKMKKFYPFY--KNEQEFLaS FNQ--------ATTSEk-- | 1296 |
| WP_010991369 | 1216 | ANCEV-----SD-GKSLD--YIESNREMFAELLAHVSEFAKRYTLAE--ANLNKIN-Q LFE-QN---KEGDIKA-- | 1274 |
| WP_033838504 | 1216 | ANCEV-----SD-GKSLD--YIESNREMFAELLAHVSEFAKRYTLAE--ANLNKIN-Q LFE-QN---KEGDIKA-- | 1274 |
| EHN60060 | 1219 | ANCEV-----SD-GKSLD--YIESNREMFAELLAHVSEFAKRYTLAE--ANLNKIN-Q LFE-QN---KEGDIKA-- | 1277 |
| EFR89594 | 985 | ANCEV-----SD-GKSLD--YIESNREMFAELLAHVSEFAKRYTLAE--ANLNKIN-Q LFE-QN---KEGDIKA-- | 1043 |
| WP_038409211 | 1216 | KNCEA-----ND-GESLA--YIEMHREMFAELLAYISEFAKRYTLAN--DRLEKIN-M FFE-QN---KKGDIKV-- | 1274 |
| EFR95520 | 835 | KNCEA-----ND-GESLA--YIEMHREMFAELLAYISEFAKRYTLAN--DRLEKIN-M FFE-QN---KKGDIKV-- | 893 |
| WP_003723650 | 1216 | KNCEA-----SD-GKSLK--YIEAHRETFSELLAQVSEFATRYTLAD--ANLSKIN-N LFE-QN---KEGDIKA-- | 1274 |
| WP_003727705 | 1216 | KNCEA-----SD-GKSLK--YIEAHRETFSELLAQVSEFATKYTLAD--ANLSKIN-N LFE-QN---KEGDIKA-- | 1274 |
| WP_003730785 | 1216 | KNCEA-----SD-GKSLK--YIEAHRETFSELLAQVSEFATKYTLAD--ANLSKIN-N LFE-QN---KEGDIKA-- | 1274 |
| WP_003733029 | 1216 | EKYEA-----ID-GESLA--YIEVHRALFDELLAYISEFARKYTLSN--DRLDEIN-M LYE-RN---KDGDVKS-- | 1274 |
| WP_003739838 | 1216 | KNCEA-----SD-GKSLD--YIESNREMFGELLAHVSEFAKRYTLAD--ANLSKIN-Q LFE-QN---KDNDIKV-- | 1274 |
| WP_014601172 | 1216 | KNCEA-----SD-GKSLK--YIEAHRETFSELLAQVSEFATRYTLAD--ANLSKIN-N LFE-QN---KEGDIQA-- | 1274 |
| WP_023548323 | 1216 | EKREA-----ID-GESLA--YIEAHKAVFGELLAHISEFARKYTLAN--DKLDEIN-M LYE-RN---KDGDVKS-- | 1274 |
| WP_031665337 | 1216 | KNCEA-----SD-GKSLK--YIEAHRETFSELLAQVSEFATRYTLAD--ANLSKIN-N LFE-QN---KEGDIKA-- | 1274 |
| WP_031669209 | 1216 | EKYEA-----ID-GESLA--YIEVHRALFDELLAYISEFARKYTLSN--DRLDEIN-M LYE-RN---KDGDVKS-- | 1274 |
| WP_033920898 | 1216 | EKREA-----ID-GESLA--YIEAHKAVFGELLAHISEFARKYTLAN--DKLDEIN-M LYE-RN---KDGDVKS-- | 1274 |
| AKI42028 | 1219 | KNCEA-----SD-GKSLK--YIEAHRETFSELLAQVSEFATRYTLAD--ANLSKIN-N LFE-QN---KEGDIQA-- | 1277 |
| AKI50529 | 1219 | EKREA-----ID-GESLA--YIEAHKAVFGELLAHISEFARKYTLAN--DKLDEIN-M LYE-RN---KDGDVKS-- | 1277 |
| EFR83390 | 664 | KNCEA-----SD-GKSLK--YTEAHRETFSELLAQVSEFATRYTLAD--ANLSKIN-N LFE-QN---KEGDIKX-- | 722 |
| WP_046323366 | 1216 | KNCEA-----SD-GKSLA--YIESHREMFAELLDSISEFASRYTLAD--ANLEKIN-T IFE-QN---KSGDVKV-- | 1274 |
| AKE81011 | 1256 | SHYEKLKgSPEDnEQKOL--FVEQHKHYLDEIIEQISEFSKRVILAD--ANLDKVL-S AYN-KH---RDKPIREq- | 1321 |
| CUO82355 | 1216 | YNAIYKQ-DFDGIDNMLMi-----------QLYLQLIDKLKTLYPIY-mGIVEKFE-K FVS----i----SKEEk- | 1272 |
| WP_033162887 | 1218 | YAAMLKK-RYEYIDEEEIf-----------DLYLQLLQKMDTLYPAY-kGIAKRFF-D FKN----i----DVVEK- | 1274 |
| AGZ01981 | 1273 | SHYEKLKgSPEDnEQKOL--FVEQHKHYLDEIIEQISEFSKRVILAD--ANLDKVL-S AYN-KH---RDKPIREq- | 1338 |
| AKA60242 | 1240 | SHYEKLKgSPEDnEQKOL--FVEQHKHYLDEIIEQISEFSKRVILAD--ANLDKVL-S AYN-KH---RDKPIREq- | 1305 |
| AKS40380 | 1240 | SHYEKLKgSPEDNEQKQL--FVEQHKHYLDEIIEQISEFSKRVILAD--ANLDKVL-S AYN-KH---RDKPIREq- | 1305 |
| 4UN5_B | 1244 | SHYEKLKgSPEDnEQKQL--FVEQHKHYLDEIIEQISEFSKRVILAD--ANLDKVL-S AYN-KH---RDKPIREq- | 1309 |
| WP_010922251 | 1306 | 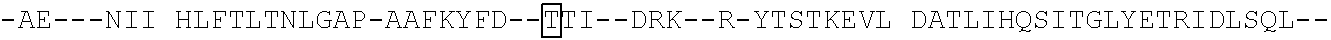 | 1365 |
| WP_039695303 | 1309 | ISN---SFI NLLTLTALGAP-ADFNFLG--EKI--PRK--R-YTSTKECL NATLIHQSITGLYETRIDLSKL-- | 1369 |
| WP_045635197 | 1307 | LAN---SFI NLLTFTALGAP-AAFKFFG--KDI--DRK--R-YTTVSEIL NATLIHQSITGLYETWIDLSKL-- | 1367 |
| 5AXW_A | | --------- ----------------------------------------- ------------------------ | |
| WP_009880683 | 990 | -AE---NII HLFTLTNLGAP-AAFKCFD--TTI--GRN--R-YKSIKEVL DATLIHQSITGLYETRIDLSQL-- | 1049 |
| WP_010922251 | 1306 | -AE---NII HLFTLTNLGAP-AAFKYFD--TTI--DRK--R-YTSTKEVL DATLIHQSITGLYETRIDLSQL-- | 1365 |
| WP_011054416 | 1306 | -AE---NII HLFTLTNLGAP-AAFKYFD--TTI--DRK--R-YTSTKEVL DATLIHQSITGLYETRIDLSQL-- | 1365 |
| WP_011284745 | 1306 | -AE---NII HLFTLTNLGAP-AAFKYFD--TTI--DRK--R-YTSTKEVL DATFIHQSITGLYETRIDLSQL-- | 1365 |
| WP_011285506 | 1306 | -AE---NII HLFTLTNLGAP-AAFKYFD--TTI--DRK--R-YTSTKEVL DATLIHQSITGLYETRIDLSQL-- | 1365 |
| WP_011527619 | 1306 | -AE---NII HLFTLTNLGAP-TAFKYFD--TTI--DRK--R-YTSTKEVL DATFIHQSITGLYETRIDLSQL-- | 1365 |
| WP_012560673 | 1306 | -AE---NII HLFTLTNLGAP-AAFKCFD--TTI--GRN--R-YKSIKEVL DATLIHQSITGLYETRIDLSQL-- | 1365 |
| WP_014407541 | 1305 | -AE---NII HLFTLTNLGAP-AAFKYFD--TTI--DRK--R-YTSTKEVL DATLIHQSITGLYETRIDLSQL-- | 1364 |
| WP_020905136 | 1306 | -AE---NII HLFTLTNLGAP-AAFKYFD--TTI--DRK--R-YTSTKEVL DATLIHQSITGLYETRIDLSQL-- | 1365 |
| WP_023080005 | 1305 | -AK---NII HLFTLTNLGAP-AAFKYFD--TTI--ERN--R-YKSIKEVL DATLIHQSITGLYETRIDLSQL-- | 1364 |
| WP_023610282 | 1305 | -AK---NII HLFTLTNLGAP-AAFKYFD--TTI--ERN--R-YKSIKEVL DATLIHQSITGLYEIRIDLSQL-- | 1364 |
| WP_030125963 | 1306 | -AE---NII HLFTLTNLGAP-AAFKYFD--TTI--GRN--R-YKSIKEVL DATLIHQSITGLYETRIDLSQL-- | 1365 |
| WP_030126706 | 1306 | -AE---NII HLFTLTNLGAP-AAFKYFD--TTI--DRK--R-YTSTKEVL DATLIHQSITGLYETRIDLSQL-- | 1365 |
| WP_031488318 | 1306 | -AE---NII HLFTLTNFGAP-AAFIYFD--TTI--GRN--R-YKSIKEVL DATLIHQSITGLYETRIDLSQL-- | 1365 |
| WP_032460140 | 1306 | -AE---NII HLFTLTNLGAP-AAFKYFD--TTI--GRN--R-YKSIKEVL DATLIHQSITGLYETRIDLSQL-- | 1365 |
| WP_032461047 | 1306 | -AE---NII HLFTLTNLGAP-AAFKYFD--TTI--GRN--R-YKSIKEVL DATLIHQSITGLYETRIDLSQL-- | 1365 |
| WP_032462016 | 1306 | -AE---NII HLFTLTNLGAP-AAFKYFD--TTI--DRK--R-YTSTKEVL DATLIHQSITGLYETRIDLSQL-- | 1365 |
| WP_032462936 | 1306 | -AE---NII HLFTLTNLGAP-AAFKYFD--TTI--DRK--R-YTSTKEVL DATLIHQSITGLYETRIDLSQL-- | 1365 |
| WP_032464890 | 1306 | -AE---NII HLFTLTNLGAP-TAFKYFD--TTI--DRK--R-YTSTKEVL DATFIHQSITGLYETRIDLSQL-- | 1365 |
| WP_033888930 | 1131 | -AE---NII HLFTLTNLGAP-AAFKYFD--TTI--DRK--R-YTSTKEVL DATLIHQSITGLYETRIDLSQL-- | 1190 |
| WP_038431314 | 1306 | -AE---NII HLFTLTNLGAP-AAFKYFD--TTI--DRK--R-YTSTKEVL DATLIHQSITGLYETRIDLSQL-- | 1365 |
| WP_038432938 | 1305 | -AK---NII HLFTLTNLGAP-AAFKYFD--TTI--ERN--R-YKSIKEVL DATLIHQSITGLYETRIDLSQL-- | 1364 |
| WP_038434062 | 1306 | -AE---NII HLFTLTNLGAP-AAFKYFD--TTI--GRN--R-YKSIKEVL DATLIHQSITGLYETRIDLSQL-- | 1365 |
| BAQ51233 | 1217 | -AE---NII HLFTLTNLGAP-AAFKYFD--TTI--DRK--R-YTSTKEVL DATLIHQSITGLYETRIDLSQL-- | 1276 |
| KGE60162 | 481 | -AE---NII HLFTLTNLGAP-AAFKYFD--TTI--DRK--R-YTSTKEVL DATLIHQSITGLYETRIDLSQL-- | 540 |
| KGE60856 | 244 | -AE---NII HLFTLTNLGAP-AAFKYFD--TTI--DRK--R-YTSTKEVL DATLIHQSITGLYETRIDLSQL-- | 303 |
| WP_002989955 | 1306 | -AE---NII HLFTLTNLGAP-TAFKYFD--TTI--DRK--R-YTSTKEVL DATFIHQSITGLYETRIDLSQL-- | 1365 |
| WP_003030002 | 1282 | LAK---SFI SLLTFTAFGAP-AAFNFFG--ENI--DRK--R-YTSVTECL NATLIHQSITGLYETRIDLSKL-- | 1342 |
| WP_003065552 | 1310 | ISN---SFI NLLTLTALGAP-ADFNFLG--EKI--PRK--R-YTSTKECL NATLIHQSITGLYETRIDLSKI-- | 1370 |
| WP_001040076 | 1307 | LAN---NII NLFTFTSLGAP-AAFKFFD--KIV--DRK--R-YTSTKEVL NSTLIHQSITGLYETRIDLGKL-- | 1367 |
| WP_001040078 | 1315 | LAN---NII NLFTFTSLGAP-AAFKFFD--KIV--DRK--R-YTSTKEVL NSTLIHQSITGLYETRIDLSKL-- | 1375 |
| WP_001040080 | 1307 | LAN---NII NLFTFTSLGAP-AAFKFFD--KIV--DRK--R-YTSTKEVL NSTLIHQSITGLYETRIDLGKL-- | 1367 |
| WP_001040081 | 1307 | LAN---NII NLFTFTSLGAP-AAFKFFD--KIV--DRK--R-YTSTKEVL NSTLIHQSITGLYETRIDLGKL-- | 1367 |
| WP_001040083 | 1307 | LAN---NII NLFTFTSLGAP-AAFKFFD--KIV--DRK--R-YTSTKEVL NSTLIHQSITGLYETRIDLGKL-- | 1367 |
| WP_001040085 | 1307 | LAN---NII NLFTFTSLGAP-AAFKFFD--KIV--DRK--R-YTSTKEVL NSTLIHQSITGLYETRIDLGKL-- | 1367 |
| WP_001040087 | 1307 | LAN---NII NLFTFTSLGAP-AAFKFFD--KIV--DRK--R-YTSTKEVL NSTLIHQSITGLYETRIDLGKL-- | 1367 |
| WP_001040088 | 1307 | LAN---NII NLFTFTSLGAP-AAFKFFD--KIV--DRK--R-YTSTKEVL NSTLIHQSITGLYETRIDLGKL-- | 1367 |
| WP_001040089 | 1307 | LAN---NII NLFTFTSLGAP-AAFKFFD--KIV--DRK--R-YTSTKEVL NSTLIHQSITGLYETRIDLGKL-- | 1367 |
| WP_001040090 | 1307 | LAN---NII NLFTFTSLGAP-AAFKFFD--KIV--DRK--R-YTSTKEVL NSTLIHQSITGLYETRIDLGKL-- | 1367 |
| WP_001040091 | 1307 | LAN---NII NLFTFTSLGAP-AAFKFFD--KIV--DRK--R-YTSTKEVL NSTLIHQSITGLYETRIDLGKL-- | 1367 |
| WP_001040092 | 1307 | LAK---NII NLFTFTSLGAP-AAFKFFD--KSV--DRK--R-YTSTKEVL DSTLIHQSITGLYETRIDLGKL-- | 1367 |
| WP_001040094 | 1307 | LAN---NII NLFTFTSLGAP-AAFKFFD--KIV--DRK--R-YTSTKEVL NSTLIHQSITGLYETRIDLGKL-- | 1367 |
| WP_001040095 | 1307 | LAN---NII NLFTFTSLGAP-AAFKFFD--KIV--DRK--R-YTSTKEVL NSTLIHQSITGLYETRIDLGKL-- | 1367 |
| WP_001040096 | 1307 | LAN---NII NLFTFTSLGAP-AAFKFFD--KIV--DRK--R-YTSTKEVL NSTLIHQSITGLYETRIDLGKL-- | 1367 |
| WP_001040097 | 130 | LAN---NII NLFTFTSLGAP-AAFKFFD--KIV--DRK--R-YTSTKEVL NSTLIHQSITGLYETRIDLGKL-- | 1367 |
| WP_001040098 | 1307 | LAN---NII NLFTFTSLGAP-AAFKFFD--KIV--DRK--R-YTSTKEVL NSTLIHQSITGLYETRIDLGKL-- | 1367 |
| WP_001040099 | 1307 | LAN---NII NLFTFTSLGAP-AAFKFFD--KIV--DRK--R-YTSTKEVL NSTLIHKSITGLYETRIDLGKL-- | 1367 |
| WP_001040100 | 1307 | LAN---NII NLFTFTSLGAP-AAFKFFD--KIV--DRK--R-YTSTKEVL NSTLIHQSITGLYETRIDLGKL-- | 1367 |
| WP_001040104 | 1307 | LAN---NII NLFTFTSLGAP-AAFKFFD--KIV--DRK--R-YTSTKEVL NSTLIHQSITGLYETRIDLGKL-- | 1367 |
| WP_001040105 | 1307 | LAN---NII NLFTFTSLGAP-AAFKFFD--KIV--DRK--R-YTSTKEVL NSTLIHQSITGLYETRIDLGKL-- | 1367 |
| WP_001040106 | 1307 | LAN---NII NLFTFTSLGAP-AAFKFFD--KIV--DRK--R-YTSTKEVL NSTLIHQSITGLYETRIDLGKL-- | 1367 |
| WP_001040107 | 1307 | LAN---NII NLFTFTSLGAP-AAFKFFD--KIV--DRK--R-YTSTKEVL NSTLIHQSITGLYETRIDLGKL-- | 1367 |
| WP_001040108 | 1307 | LAN---NII NLFTFTSLGAP-AAFKFFD--KIV--DRK--R-YTSTKEVL NSTLIHQSITGLYETRIDLGKL-- | 1367 |
| WP_001040109 | 1307 | LAN---NII NLFTFTSLGAP-AAFKFFD--KIV--DRK--R-YTSTKEVL NSTLIHQFITGLYETRIDLGKL-- | 1367 |
| WP_001040110 | 1307 | LAN---NII NLFTFTSLGAP-AAFKFFD--KIV--DRK--R-YTSTKEVL NSTLIHQSITGLYETRIDLGKL-- | 1367 |
| WP_015058523 | 1307 | LAK---NII NLFTFTSLGAP-AAFKFFD--KSV--DRK--R-YTSTKEVL DSTLIHQSITGLYETRIDLGKL-- | 1367 |
| WP_017643650 | 1307 | LAN---NII NLFTFTSLGAP-AAFKFFD--KIV--DRK--R-YTSTKEVL NSTLIHQSITGLYETRIDLGKL-- | 1367 |
| WP_017647151 | 1307 | LAN---NII NLFTFTSLGAP-AAFKFFD--KIV--DRK--R-YTSTKEVL NSTLIHQSITGLYETRIDLGKL-- | 1367 |
| WP_017648376 | 1307 | LAN---NII NLFTFTSLGAP-AAFKFFD--KIV--DRK--R-YTSTKEVL NSTLIHQSITGLYETRIDLGKL-- | 1367 |
| WP_017649527 | 1307 | LAN---NII NLFTFTSLGAP-AAFKFFD--KIV--DRK--R-YTSTKEVL NSTLIHQSITGLYETRIDLGKL-- | 1367 |
| WP_017771611 | 1307 | LAN---NII NLFTFTSLGAP-AAFKFFD--KIV--DRK--R-YTSTKEVL NSTLIHQSITGLYETRIDLGKL-- | 1367 |
| WP_017771984 | 1307 | LAN---NII NLFTFTSLGAP-AAFKFFD--KIV--DRK--R-YTSTKEVL NSTLIHQSITGLYETRIDLGKL-- | 1367 |
| CFQ25032 | 1307 | LAN---NII NLFTFTSLGAP-AAFKFFD--KIV--DRK--R-YTSTKEVL NSTLIHQSITGLYETRIDLGKL-- | 1367 |
| CFV16040 | 1307 | LAN---NII NLFTFTSLGAP-AAFKFFD--KIV--DRK--R-YTSTKEVL NSTLIHQSITGLYETRIDLGKL-- | 1367 |
| KLJ37842 | 1307 | LAN---NII NLFTFTSLGAP-AAFKFFD--KIV--DRK--R-YTSTKEVL NSTLIHQSITGLYETRIDLGKL-- | 1367 |
| KLJ72361 | 1307 | LAN---NII NLFTFTSLGAP-AAFKFFD--KIV--DRK--R-YTSTKEVL NSTLIHQSITGLYETRIDLGKL-- | 1367 |
| KLL20707 | 1321 | LAN---NII NLFTFTSLGAP-AAFKFFD--KIV--DRK--R-YTSTKEVL NSTLIHQSITGLYETRIDLGKL-- | 1381 |
| KLL42645 | 1307 | LAN---NII NLFTFTSLGAP-AAFKFFD--KIV--DRK--R-YTSTKEVL NSTLIHQSITGLYETRIDLGKL-- | 1367 |
| WP_047207273 | 1307 | LAN---NII NLFTFTSLGAP-AAFKFFD--KIV--DRK--R-YTSTKEVL NSTLIHQSITGLYETRIDLGKL-- | 1367 |
| WP_047209694 | 1307 | LAN---NII NLFTFTSLGAP-AAFKFFD--KIV--DRK--R-YTSTKEVL NSTLIHKSITGLYETRIDLGKL-- | 1367 |
| WP_050198062 | 1307 | LAN---NII NLFTFTSLGAP-AAFKFFD--KIV--DRK--R-YTSTKEVL NSTLIHQSITGLYETRIDLGKL-- | 1367 |
| WP_050201642 | 1307 | LAN---NII NLFTFTSLGAP-AAFKFFD--KIV--DRK--R-YTSTKEVL NSTLIHQSITGLYETRIDLGKL-- | 1367 |
| WP_050204027 | 1307 | LAN---NII NLFTFTSLGAP-AAFKFFD--KIV--DRK--R-YTSTKEVL NSTLIHQSITGLYETRIDLGKL-- | 1367 |
| WP_050881965 | 1307 | LAN---NII NLFTFTSLGAP-AAFKFFD--KII--DRK--R-YTSTKEVL NSTLIHQSITGLYETRIDLGKL-- | 1367 |
| WP_050886065 | 1307 | LAN---NII NLFTFTSLGAP-AAFKFFD--KIV--DRK--R-YTSTKEVL NSTLIHQSITGLYETRIDLGKL-- | 1367 |
| AHN30376 | 1307 | LAK---NII NLFTFTSLGAP-AAFKFFD--KSV--DRK--R-YTSTKEVL DSTLIHQSITGLYETRIDLGKL-- | 1367 |
| EAO78426 | 1307 | LAN---NII NLFTFTSLGAP-AAFKFFD--KIV--DRK--R-YTSTKEVL NSTLIHQSITGLYETRIDLGKL-- | 1367 |
| CCW42055 | 1307 | LAN---NII NLFTFTSLGAP-AAFKFFD--KIV--DRK--R-YTSTKEVL NSTLIHQSITGLYETRIDLGKL-- | 1367 |
| WP_003041502 | 1314 | ICT---SFL GLFELTSLGSA-SDFEFLG--VKI--PRY--RdYTPSSLLK DSTLIHQSITGLYETRIDLSKL-- | 1383 |
| WP_037593752 | 1283 | LAK---SFI SLLTFTAFGAP-AAFNFFG--ENI--DRK--R-YTSVTECL NATLIHQSITGLYETRIDLSKL-- | 1343 |
| WP_049516684 | 1283 | LAK---SFI SLLTFTAFGAP-AAFNFFG--ENI--DRK--R-YTSVTECL NATLIHQSITGLYETRIDLSKL-- | 1343 |
| GAD46167 | 1282 | LAK---SFI SLLTFTAFGAP-AAFNFFG--ENI--DRK--R-YTSVTECL NATLIHQSITGLYETRIDLSKL-- | 1342 |
| WP_018363470 | 1314 | ISD---SFI NLLTLTALGAP-ADFNFLG--EKI--PRK--R-YNSTKECL NATLIHQSITGLYETRIDLSKL-- | 1374 |
| WP_003043819 | 1311 | -SN---SFV SLLKYTSFGAS-GGFTFLD--LDVkqGRL--R-YQTVTEVL DATLIYQSITGLYETRTDLSQL-- | 1372 |
| WP_006269658 | 1282 | LAK---SFI SLLTFTAFGAP-AAFNFFG--ENI--DRK--R-YTSVTECL NATLIHQSITGLYETRIDLSKL-- | 1342 |
| WP_048800889 | 1302 | ISN---SFI HLLTLTALGAP-ADFNFLG--EKI--PRK--R-YTSTKECL NATLIHQSITGLYETQTDLSKL-- | 1362 |
| WP_012767106 | 1309 | -CS---SVI NLLTLTASGAP-ADFKFLG--TTI--PRK--R-YGSPQSIL SSTLIHQSITGLYETRIDLSQL-- | 1368 |
| WP_014612333 | 1309 | -CS---SVI NLLTLTASGAP-ADFKFLG--TTI--PRK--R-YGSPQSIL SSTLIHQSITGLYETRIDLSQL-- | 1368 |
| WP_015017095 | 1309 | -CS---SVI NLLTLTASGAP-ADFKFLG--TTI--PRK--R-YGSPQSIL SSTLIHQSITGLYETRIDLSQL-- | 1368 |
| WP_015057649 | 1309 | -CS---SVI NLLTLTASGAP-ADFKFLG--TTI--PRK--R-YGSPQSIL SSTLIHQSITGLYETRIDLSQL-- | 1368 |
| WP_048327215 | 1309 | -CS---SVI NLLTLTASGAP-ADFKFLG--TTI--PRK--R-YGSPQSIL SSTLIHQSITGLYETRIDLSQL-- | 1368 |
| WP_049519324 | 1309 | -CS---SVI NLLTLTASGAP-ADFKFLG--TTI--PRK--R-YGSPQSIL SSTLIHQSITGLYETRIDLSQL-- | 1368 |
| WP_012515931 | 1286 | -AE---NII NVFTFVALGAP-AAFKFFD--ATI--DRK--R-YTSTKEVL NATLIHQSVTGLYETRIDLSQL-- | 1345 |
| WP_021320964 | 1286 | -AE---NII NVFTFVALGAP-AAFKFFD--ATI--DRK--R-YTSTKEVL NATLIHQSVTGLYETRIDLSQL-- | 1345 |
| WP_037581760 | 1286 | -AE---NII NVFTFVALGAP-AAFKFFD--ATI--DRK--R-YTSTKEVL NATLIHQSVTGLYETRIDLSQL-- | 1345 |
| WP_004232481 | 1309 | ISSlseSFI NLLKFISFGAP-GAFKFLK--LDV--KQSnlR-YKSTTEAL SATLIHQSVTGLYETRIDLSKI-- | 1374 |
| WP_009854540 | 1307 | ISN---SFI NLLTLTALGAP-ADFNFLG--EKI--PRK--R-YTSTKECL TATLIHQSITGLYETRIDLSKL-- | 1367 |
| WP_012962174 | 1308 | ISI---SFV NLLTLTALGAP-ADFNFLG--EKI--PRK--R-YTSTKECL NATLIHQSITGLYETRIDLSKL-- | 1368 |
| WP_039695303 | 1309 | ISN---SFI NLLTLTALGAP-ADFNFLG--EKI--PRK--R-YTSTKECL NATLIHQSITGLYETRIDLSKL-- | 1369 |
| WP_014334983 | 1312 | ISA---SFI NLLTLTALGAP-ADFNFLG--EKI--PRK--R-YTSTKECL SATLIHQSVTGLYETRIDLSKL-- | 1372 |
| WP_003099269 | 1306 | -AI---NML NLFTFTDLGAP-SAFKFFN--GDI--DRK--R-YSSTNEII NSTLIYQSPTGLYETRIDLSKL-- | 1365 |
| AHY15608 | | --------- ----------------------------------------- ------------------------ | |
| AHY17476 | | --------- ----------------------------------------- ------------------------ | |
| ESR09100 | 138 | -AI---NML NLFTFTDLGAP-SAFKFFNg--DI--DRK--R-YSSTNEII NSTLIYQSPTGLYETRIDLSKL-- | 197 |
| AGM98575 | | --------- ----------------------------------------- ------------------------ | |
| ALF27331 | 1282 | LAS---SFI NLLTFTAIGAP-AAFKFFD--NNI--DRK--R-YTSTTEIL NATLIHQSITGLYETRIDLSRL-- | 1342 |
| WP_018372492 | 1290 | VAR---SFV -LLNFTMMGAA-TDFKFFG--QII--PRK--R-YPSTTECL KSTLIHQSVTGLYETRIDLSKL-- | 1350 |
| WP_045618028 | 1311 | LSE---SFI SLLKLISFGAP-GTFKFLG--VEI--SQSnvR-YQSVSSCF NATLIHQSITGLYETRIDLSKL-- | 1373 |
| WP_045635197 | 1307 | LAN---SFI NLLTFTALGAP-AAFKFFG--KDI--DRK--R-YTTVSEIL NATLIHQSITGLYETWIDLSKL-- | 1367 |
| WP_002263549 | 1282 | LAS---SFI NLLTFTAIGAP-ATFKFFD--KNI--DRK--R-YTSTTEIL NATLIHQSITGLYETRIDLNKL-- | 1342 |
| WP_002263887 | 1282 | LAS---SFI NLLTFTAIGAP-ATFKFFD--KNI--DRK--R-YTSTTEIL NATLIHQSITGLYETRIDLNKL-- | 1342 |
| WP_002264920 | 1282 | LAS---SFI NLLTFTAIGAP-ATFKFFD--KNI--DRK--R-YTSTTEIL NATLIHQSITGLYETRIDLSKL-- | 1342 |
| WP_002269043 | 1282 | LAS---SFI NLLTFTAIGAP-ATFKFFD--KNI--DRK--R-YTSTTEIL NATLIHQSITGLYETRIDLNKL-- | 1342 |
| WP_002269448 | 1282 | LAS---SFI NLLTFTAIGAP-ATFKFFD--KNI--DRK--R-YTSTTEIL NATLIHQSITGLYETRIDLSKL-- | 1342 |
| WP_002271977 | 1282 | LAS---SFI NLLTFTAIGAP-ATFKFFD--KNI--DRK--R-YTSTTEIL NATLIHQSITGLYETRIDLSKL-- | 1342 |
| WP_002272766 | 1282 | LAS---SFI NLLTFTAIGAP-ATFKFFD--KNI--DRK--R-YTSTTEIL NATLIHQSITGLYETRIDLSKL-- | 1342 |
| WP_002273241 | 1282 | LAS---SFI NLLTFTAIGAP-ATFKFFD--KNI--DRK--R-YTSTTEIL NATLIHQSITGLYETRIDLSKL-- | 1342 |
| WP_002275430 | 1282 | LSS---SFI NLLTFTAIGAP-AAFKFFD--KNI--DRK--R-YTSTTEIL NATLIHQSITGLYETRIDLSKL-- | 1342 |
| WP_002276448 | 1282 | LAS---SFI NLLTFTAIGAP-ATFKFFD--KNI--DRK--R-YTSTTEIL NATLIHQSITGLYETRIDLSKL-- | 1342 |
| WP_002277050 | 1288 | LAD---GFI KLLGFTQLGAT-SPFSFLG--IKL--NQK--Q-YTGKKDYL EATLIHQSITGLYETRIDLNKL-- | 1352 |
| WP_002277364 | 1282 | LAS---SFI NLLTFTAIGAP-ATFKFFD--KNI--DRK--R-YTSTTEIL NATLIHQSITGLYETRIDLSKL-- | 1342 |
| WP_002279025 | 1282 | LAS---SFI NLLTFTAIGAP-ATFKFFD--KNI--DRK--R-YTSTTEIL NATLIHQSITGLYETRIDLSKL-- | 1342 |
| WP_002279859 | 1282 | LAS---SFI NLLTFTAIGAP-ATFKFFD--KNI--DRK--R-YTSTTEIL NATLIHQSITGLYETRIDLSKL-- | 1342 |
| WP_002280230 | 1282 | LAS---SFI NLLTFTAIGAP-ATFKFFD--KNI--DRK--R-YTSTTEIL NATLIHQSITGLYETRIDLSKL-- | 1342 |
| WP_002281696 | 1282 | LSS---SFI NLLTFTAIGAP-AAFKFFD--KNI--DRK--R-YTSTTEIL NATLIHQSITGLYETRIDLSKL-- | 1342 |
| WP_002282247 | 1288 | LAD---GFI KLLGFTQLGAT-SPFSFLG--IKL--NQK--Q-YTGKKDYL EATLIHQSITGLYETRIDLSKL-- | 1352 |
| WP_002282906 | 1282 | LAS---SFI NLLTFTAIGAP-ATFKFFD--KNI--DRK--R-YTSTTEIL NATLIHQSITGLYETRIDLSKL-- | 1342 |
| WP_002283846 | 1282 | LAS---SFI NLLTFTAIGAP-ATFKFFD--KNI--DRK--R-YTSTTEIL NATLIHQSITGLYETRIDLSKL-- | 1342 |
| WP_002287255 | 1282 | LAS---SFI NLLTFTAIGAP-ATFKFFD--KNI--DRK--R-YTSTTEIL NATLIHQSITGLYETRIDLSKL-- | 1342 |
| WP_002288990 | 1282 | LAS---SFI NLLTFTAIGAP-AAFKFFD--KNI--DRK--R-YTSTTEIL NATLIHQSITGLYETRIDLSKL-- | 1342 |
| WP_002289641 | 1282 | LAS---SFI NLLTFTAIGAP-ATFKFFD--KNI--DRK--R-YTSTTEIL NATLIHQSITGLYETRIDLSKL-- | 1342 |
| WP_002290427 | 1282 | LAS---SFI NLLTFTAIGAP-ATFKFFD--KNI--DRK--R-YTSTTEIL NATLIHQSITGLYETRIDLSKL-- | 1342 |
| WP_002295753 | 1282 | LAS---SFI NLLTFTAIGAP-AAFKFFD--KNI--DRK--R-YTSTTEIL NATLIHQSITGLYETRIDLSKL-- | 1342 |
| WP_002296423 | 1282 | LAS---SFI NLLTFTAIGAP-ATFKFFD--KNI--DRK--R-YTSTTEIL NATLIHQSITGLYETRIDLSKL-- | 1342 |
| WP_002304487 | 1296 | LAS---SFI NLLTFTAIGAP-AAFKFFD--KNI--DRK--R-YTSTTEIL NATLIHQSITGLYETRIDLNKL-- | 1356 |
| WP_002305844 | 1282 | LAS---SFI NLLTFTAIGAP-ATFKFFD--KNI--DRK--R-YTSTTEIL NATLIHQSITGLYETRIDLSKL-- | 1342 |
| WP_002307203 | 1282 | LAS---SFI NLLTFTAIGAP-ATFKFFD--KNI--DRK--R-YTSTTEIL NATLIHQSITGLYETRIDLSKL-- | 1342 |
| WP_002310390 | 1282 | LAS---SFI NLLTFTAIGAP-ATFKFFD--KNI--DRK--R-YTSTTEIL NATLIHQSITGLYETRIDLSKL-- | 1342 |
| WP_002352408 | 1282 | LAS---SFI NLLTFTAIGAP-ATFKFFD--KNI--DRK--R-YTSTTEIL NATLIHQSITGLYETRIDLSKL-- | 1342 |
| WP_012997688 | 1282 | LAS---SFI NLLTFTAIGAP-ATFKFFD--KNI--DRK--R-YTSTTEIL NATLIHQSITGLYETRIDLSKL-- | 1342 |
| WP_014677909 | 1282 | LAS---SFI NLLTFTAIGAP-ATFKFFD--KNI--DRK--R-YTSTTEIL NATLIHQSITGLYETRIDLSKL-- | 1342 |
| WP_019312892 | 1282 | LAS---SFI NLLTFTAIGAP-ATFKFFD--KNI--DRK--R-YTSTTEIL NATLIHQSITGLYETRIDLSKL-- | 1342 |
| WP_019313659 | 1282 | LAS---SFI NLLTFTAIGAP-ATFKFFD--KNI--DRK--R-YTSTTEIL NATLIHQSITGLYETRIDLNKL-- | 1342 |
| WP_019314093 | 1282 | LAS---SFI NLLTFTAIGAP-AAFKFFD--KNI--DRK--R-YTSTTEIL NATLIHQSITGLYETRIDLSKL-- | 1342 |
| WP_019315370 | 1282 | LSS---SFI NLLTFTAIGAP-AAFKFFD--KNI--DRK--R-YTSTTEIL NATLIHQSITGLYETRIDLSKL-- | 1342 |
| WP_019803776 | 1282 | LAS---SFI NLLTFTAIGAP-ATFKFFD--KNI--DRK--R-YTSTTEIL NATLIHQSITGLYETRIDLNKL-- | 1342 |
| WP_019805234 | 1282 | LAS---SFI NLLTFTAIGAP-ATFKFFD--KNI--DRK--R-YTSTTEIL NATLIHQSITGLYETRIDLSKL-- | 1342 |
| WP_024783594 | 1282 | LAS---SFI NLLTFTAIGAP-ATFKFFD--KNI--DRK--R-YTSTTEIL NATLIHQSITGLYETRIDLNKL-- | 1342 |
| WP_024784288 | 1288 | LAD---GFI KLLGFTQLGAT-SPFSFLG--IKL--NQK--Q-YTGKKDYL EATLIHQSITGLYETRIDLSKL-- | 1352 |
| WP_024784666 | 1282 | LAS---SFI NLLTFTAIGAP-ATFKFFD--KNI--DRK--R-YTSTTEIL NATLIHQSITGLYETRIDLSKL-- | 1342 |
| WP_024784894 | 1282 | LAS---SFI NLLTFTAIGAP-ATFKFFD--KNI--DRK--R-YTSTTEIL NATLIHQSITGLYETRIDLSKL-- | 1342 |
| WP_024786433 | 1288 | LAD---GFI KLLGFTQLGAT-SPFSFLG--IKL--NQK--Q-YTGKKDYL EATLIHQSITGLYETRIDLSKL-- | 1352 |
| WP_049473442 | 1282 | LAS---SFI NLLTFTAIGAP-ATFKFFD--KNI--DRK--R-YTSTTEIL NATLIHQSITGLYETRIDLSKL-- | 1342 |
| WP_049474547 | 1282 | LAS---SFI NLLTFTAIGAP-ATFKFFD--KNI--DRK--R-YTSTTEIL KATLIHQSITGLYETRIDLSKL-- | 1342 |
| EMC03581 | 1275 | LAS---SFI NLLTFTAIGAP-ATFKFFD--KNI--DRK--R-YTSTTEIL NATLIHQSITGLYETRIDLSKL-- | 1335 |
| WP_000428612 | 1310 | LAN---SFI NLLTFTALGAP-AAFKFFG--KDV--DRK--R-YTTVSEIL NATLIHQSITGLYETRIDLSKL-- | 1370 |
| WP_000428613 | 1308 | LAN---SFI NLLTFTALGAP-AAFKFFG--KDI--DRK--R-YTTVSEIL NATLIHQSITGLYETRIDLSKL-- | 1368 |
| WP_049523028 | 1303 | IAN---SFI NLLTFTAFGAP-AVFKFFG--KDI--ERK--R-YSTVTEIL KATLIHQSLTGLYETRIDLSKL-- | 1363 |
| WP_003107102 | 1275 | -AT---NML NLFTFTGLGAP-ATLKFFN--VDI--DRK--R-YTSSTEIL NSTLIRQSITGLYETRIDLSKI-- | 1334 |
| WP_054279288 | 1304 | -SI---SFL NLFKFTSFGAP-EKFTFLN--SEIkqDDV--R-YRSTKECL NSTLIHQSVTGLYETRIDLSQF-- | 1365 |
| WP_049531101 | 1311 | LSE---SFI SLLKLTSFGAP-GAFRFLG--VEI--SQSnvR-YQSVSSCF NATLIHQSITGLYETRIDLSKL-- | 1373 |
| WP_049538452 | 1311 | LSE---SFI SLLKLTSFGAP-GAFKFLG--VEI--SQSsvR-YKPNSQFL DATLIHQSITGLYETRIDLSKL-- | 1373 |
| WP_049549711 | 1313 | LSE---SFI SLLKLTSFGAP-GAFKFLG--AEI--SQSsvR-YKPNSQFL DTTLIHQSITGLYETRIDLSKL-- | 1375 |
| WP_007896501 | 1312 | -AL---NML NLFIFTSLGAP-STFVFFD--ETI--DRK--R-YTTSSDVL NGILIQQSITGLYETRIDLSRF-- | 1371 |
| EFR44625 | 1264 | -AL---NML NLFIFTSLGAP-STFVFFD--ETI--DRK--R-YTTSSDVL NGILIQQSITGLYETRIDLSRF-- | 1323 |
| WP_002897477 | 1307 | LAN---SFI NLLTFTALGAP-AAFKFFG--KDV--DRK--R-YTTVSEIL NATLIHQSITGLYETRIDLSKL-- | 1367 |
| WP_002906454 | 1312 | LSE---SFI SLLKLTSFGAP-GAFKFLG--VEI--SQSsvR-YKPNSQFL DTTLIHQSITGLYETRIDLSKL-- | 1374 |
| WP_009729476 | 1308 | LAN---SFI NLLTFTALGAP-AAFKFFG--KDV--DRK--R-YTTVSEIL NATLIHQSITGLYETRIDLSKL-- | 1368 |
| CQR24647 | 1296 | LAQ---SFI SLLKFTAFGAP-GGFKFLD--ADI--KQSn1R-YQTVTEVL SSTLIHQSVTGLYETRIDLSKL-- | 1358 |
| WP_000066813 | 1312 | LAN---SFI NLLTFTALGAP-AAFKFLG--KDV--DRK--R-YTTVSEIL NATLIHQSITGLYETRIDLSKL-- | 1372 |
| WP_009754323 | 1308 | LAN---SFI NLLTFTALGAP-AAFKFFG--KDV--DRK--R-YTTVSEIL NATLIHQSITGLYETRIDLSKL-- | 1368 |
| WP_044674937 | 1301 | LTS---SFV NLLTFTAIGAP-AAFKFLG--SVI--DRK--R-YTSIAEIL EATLIHQSVTGLYETRIDLSKL-- | 1361 |
| WP_044676715 | 1303 | LTS---SFV NLLTFTAIGAP-AAFKFLG--SVI--DRK--R-YTSIAEIL EATLIHQSVTGLYETRIDLSKL-- | 1363 |
| WP_044680361 | 1303 | LTS---SFV NLLTFTAIGAP-AAFKFLG--SVI--DRK--R-YTSIAEIL EATLIHQSVTGLYETRIDLSKL-- | 1363 |
| WP_044681799 | 1301 | LTS---SFV NLLTFTAIGAP-AAFKFLG--SVI--DRK--R-YTSIAEIL EATLIHQSVTGLYETRIDLSKL-- | 1361 |
| WP_049533112 | 1314 | ICT---SFL GLFELTSLGSA-SDFEFLG--VKI--PRY--RdYTPSSLLK DSTLIHQSITGLYETRIDLSKL-- | 1383 |
| WP_029090905 | 1241 | -VK----VI ELLKITQANATnGDLKLLK----M-sNREg-R-LGSVSVAL DFKI-INQSVTGLYQSIEDYNN-- | 1300 |
| WP_006506696 | 1269 | -AN----II QMLIVMHRGPQnGNIVYDDf--KI-sDRIg-R-LKTKNHNL NIVFISQSPTGIYTKKYKL----- | 1329 |
| AIT42264 | 1306 | -AE---NII HLFTLTNLGAP-AAFKYFD--TTI--DRK--R-YTSTKEVL DATLIHQSITGLYETRIDLSQL-- | 1365 |
| WP_034440723 | 1277 | LVE---SFI NLLAITKCGPA-ADITFLG--EKI--SRK--R-YRSINCLW GSEVIFQSPTGLYETRLRLE---- | 1335 |
| AKQ21048 | 1306 | -AE---NII HLFTLTNLGAP-AAFKYFD--TTI--DRK--R-YTSTKEVL DATLIHQSITGLYETRIDLSQL-- | 1365 |
| WP_004636532 | 1272 | TVE---SFV NLMTFTAMGAP-ATFKFYG--ESI--TRS--R-YTSITEFR GSTLIFQSITGLYETRYKL----- | 1329 |
| WP_002364836 | 1278 | IAA---SFI QLMQFNAMGAP-STFKFFQ--KDI--ERA--R-YTSIKEIF DATIIYQSTTGLYETRRKV----- | 1335 |
| WP_016631044 | 1229 | IAA---SFI QLMQFNAMGAP-STFKFFQ--KDI--ERA--R-YTSIKEIF DATIIYQSPTGLYETRRKV----- | 1286 |
| EMS75795 | 1014 | LSQ---SFI NLMQLNAMGAP-ADFKFFD--VII--PRK--R-YPSLTEIW ESTIIYQSITGLRETRTRMATLwd | 1076 |
| WP_002373311 | 1278 | IAA---SFI QLMQFNAMGAP-STFKFFQ--KDI--ERA--R-YTSIKEIF DATIIYQSTTGLYETRRKV----- | 1335 |
| WP_002378009 | 1278 | IAA---SFI QLMQFNAMGAP-STFKFFQ--KDI--ERA--R-YTSIKEIF DATIIYQSTTGLYETRRKV----- | 1335 |
| WP_002407324 | 1278 | IAA---SFI QLMQFNAMGAP-STFKFFQ--KDI--ERA--R-YTSIKEIF DATIIYQSTTGLYETRRKV----- | 1335 |
| WP_002413717 | 1278 | IAA---SFI QLMQFNAMGAP-STFKFFQ--KDI--ERA--R-YTSIKEIF DATIIYQSTTGLYETRRKV----- | 1335 |
| WP_010775580 | 1280 | IAA---SFI QLMQFNAMGAP-STFKFFQ--KDI--ERA--R-YTSIKEIF DATIIYQSTTGLYETRRKV----- | 1337 |
| WP_010818269 | 1278 | IAA---SFI QLMQFNAMGAP-STFKFFQ--KDI--ERA--R-YTSIKEIF DATIIYQSTTGLYETRRKV----- | 1335 |
| WP_010824395 | 1278 | IAA---SFI QLMQFNAMGAP-STFKFFQ--KDI--ERA--R-YTSIKEIF DATIIYQSTTGLYETRRKV----- | 1335 |
| WP_016622645 | 1278 | IAA---SFI QLMQFNAMGAP-STFKFFQ--KDI--ERA--R-YTSIKEIF DATIIYQSTTGLYETRRKV----- | 1335 |
| WP_033624816 | 1278 | IAA---SFI QLMQFNAMGAP-STFKFFQ--KDI--ERA--R-YTSIKEIF DATIIYQSTTGLYETRRKV----- | 1335 |
| WP_033625576 | 1278 | IAA---SFI QLMQFNAMGAP-STFKFFQ--KDI--ERA--R-YTSIKEIF DATIIYQSTTGLYETRRKV----- | 1335 |
| WP_033789179 | 1278 | IAA---SFI QLMQFNAMGAP-STFKFFQ--KDI--ERA--R-YTSIKEIF DATIIYQSTTGLYETRRKV----- | 1335 |
| WP_002310644 | 1280 | LAS---SFV NLMQFNAMGAP-ADFKFFD--VTI--PRK--R-YTSLTEIW QSTIIHQSITGLYETRIRMGK--- | 1339 |
| WP_002312694 | 1281 | LAS---SFV NLMQFNAMGAP-ADFKFFD--VTI--PRK--R-YTSLTEIW QSTIIHQSITGLYETRIRMGK--- | 1340 |
| WP_002314015 | 1281 | LAS---SFV NLMQFNAMGAP-ADFKFFD--VTI--PRK--R-YTSLTEIW QSTIIHQSITGLYETRIRMGK--- | 1340 |
| WP_002320716 | 1281 | LAS---SFV NLMQFNAMGAP-ADFKFFD--VTI--PRK--R-YTSLTEIW QSTIIHQSITGLYETRIRMGK--- | 1340 |
| WP_002330729 | 1280 | LAS---SFV NLMQFNAMGAP-ADFKFFD--VTI--PRK--R-YTSLTEIW QSTIIHQSITGLYETRIRMGK--- | 1339 |
| WP_002335161 | 1281 | LAS---SFV NLMQFNAMGAP-ADFKFFD--VTI--PRK--R-YTSLTEIW QSTIIHQSITGLYETRIRMGK--- | 1340 |
| WP_002345439 | 1281 | LAS---SFV NLMQFNAMGAP-ADFKFFD--VTI--PRK--R-YTSLTEIW QSTIIHQSITGLYETRIRMGK--- | 1340 |
| WP_034867970 | 1271 | IAQ---SFL QLLQFNAIGAP-ADFKFFG--VTI--PRK--R-YTSLTEIW DATIIYQSVTGLYETRIRMGDLwa | 1333 |
| WP_047937432 | 1281 | LAS---SFV NLMQFNAMGAP-ADFKFFD--VTI--PRK--R-YTSLTEIW QSTIIHQSITGLYETRIRMGK--- | 1340 |
| WP_010720994 | 1271 | IAQ---SFL QLLOFNAIGAP-ADFKFFG--VTI--PRK--R-YTSLTEIW DATIIYQSVTGLYETRIRMGDLwa | 1333 |
| WP_010737004 | 1271 | IAQ---SFL QLLQFNAIGAP-ADFKFFG--VTI--PRK--R-YTSLTEIW DATIIYQSVTGLYETRIRMGDLwa | 1333 |
| WP_034700478 | 1271 | IAQ---SFL QLLQFNAIGAP-ADFKFFG--VTI--PRK--R-YTSLTEIW DATIIYQSVTGLYETRIRMGDLwa | 1333 |
| WP_007209003 | 1270 | IAK---SFI NLLTFTAMGAP-ADFEFFG--EKI--PRK--R-YVSISEII DAVFIHQSITGLYETRVRLTEV-- | 1330 |
| WP_023519017 | 1265 | MID---AIL SLLKFTLFGAS-VEFKFFD--IKI---LK--R-YKSLTDIW EATIIYQSVTGLYERRVEVRKLwd | 1326 |
| WP_010770040 | 1275 | IAE---SFV NLMKFSAYGAP-MDFKFFG--KTI--PRS--R-YTSVGELL SATIINQSITGLYETRRKL----- | 1332 |
| WP_048604708 | 1271 | IAQ---SFV DLMQLNAFGAP-ADFKFFG--ETI--PRK--R-YTSVNELL EATIINQSITGLYETRRRL----- | 1328 |
| WP_010750235 | 1274 | MAH---SFV NLMQFNALGAP-ADFKFFD--TTI--TRK--R-YTSLTEIW QSTIIYQSVTGLYETRRRMADLwd | 1336 |
| AII16583 | 1345 | -AE---NII HLFTLTNLGAP-AAFKYFD--TTI--DRK--R-YTSTKEVL DATLIHQSITGLYETRIDLSQL-- | 1404 |
| WP_029073316 | 1283 | -CE----VI QMLVVMHAGPQnGNITFDDf--KL-sNRLg-R-LNCKTISL TTVFIADSPTGMYSKKYKL----- | 1343 |
| WP_031589969 | 1284 | -CN----II QILATLHCNSSIGKIMYSDf--KI-sTTIg-R-LNGRTISL DISFIAESPTGMYSKKYKL----- | 1344 |
| KDA45870 | 1254 | LGK---NFV ELLRYTADGAA-SDFKFFG--ENI--PRK--R-YNSAGSLL NGTLIYQSKTGLYETRIDLGKL-- | 1314 |
| WP_039099354 | 1307 | ILDr----V -LIGLHANAAV-SDLGVLKisTPL--GKM--Q---QPSGIS DTQIIYQSPTGLFERRVALRDL-- | 1368 |
| AKP02966 | 1297 | INS1-eELI TLLHANSTSAH-LIFNNIE-KKAF--GRK-------THGLT DTDFIYQSVTGLYETRIHIE---- | 1356 |
| WP_010991369 | 1275 | IAQ---SFV DLMAFNAMGAP-ASFKFFE--TTI--ERK--R-YNNLKELL NSTIIYQSITGLYESRKRL----- | 1332 |
| WP_033838504 | 1275 | IAQ---SFV DLMAFNAMGAP-ASFKFFE--TTI--ERK--R-YNNLKELL NSTIIYQSITGLYESRKRL----- | 1332 |
| EHN60060 | 1278 | IAQ---SFV DLMAFNAMGAP-ASFKFFE--TTI--ERK--R-YNNLKELL NSTIIYQSITGLYESRKRL----- | 1335 |
| EFR89594 | 1044 | IAQ---SFV DLMAFNAMGAP-ASFKFFE--TTI--ERK--R-YNNLKELL NSTIIYQSITGLYESRKRL----- | 1101 |
| WP_038409211 | 1275 | IAK---SFD KLKVFNAFGAP-RDFEFFE--TTI--KRK--R-YYNIKELL NATIIYQSITGLYEARKRL----- | 1332 |
| EFR95520 | 894 | IAK---SFD KLKVFNAFGAP-RDFEFFE--TTI--KRK--R-YYNIKELL NATIIYQSITGLYEARKRL----- | 951 |
| WP_003723650 | 1275 | IAQ---SFV DLMAFNAMGAP-ASFKFFE--ATI--DRK--R-YTNLKELL SSTIIYQSITGLYESRKRL----- | 1332 |
| WP_003727705 | 1275 | IAQ---SFV DLMAFNAMGAP-ASFKFFE--ATI--DRK--R-YTNLKELL SSTIIYQSITGLYESRKRL----- | 1332 |
| WP_003730785 | 1275 | IAQ---SFV DLMAFNAMGAP-ASFKFFE--ATI--DRK--R-YTNLKELL SSTIIYQSITGLYESRKRL----- | 1332 |
| WP_003733029 | 1275 | IAE---SFV SLKKFNAFGVH-QDFSFFG--TKI--ERK--R-DRKLNELL NSTIIYQSITGLYESRKRL----- | 1332 |
| WP_003739838 | 1275 | IAQ---SFV NLMAFNAMGAP-ASFKFFE--ATI--ERK--R-YTNLKELL SATIIYQSITGLYEARKRI----- | 1332 |
| WP_014601172 | 1275 | IAQ---SFV DLMAFNAMGAP-ASFKFFE--ATI--DRK--R-YTNLKELL SSTIIYQSITGLYESRKRL----- | 1332 |
| WP_023548323 | 1275 | IAE---SFV SLKKFNAFGVH-KDFNFFG--TTI--KRK--R-DRKLKELL NSTIIYQSITGLYESRKRL----- | 1332 |
| WP_031665337 | 1275 | IAQ---SFV DLMAFNAMGAP-ASFKFFE--ATI--DRK--R-YTNLKELL SSTIIYQSITGLYESRKRI----- | 1332 |
| WP_031669209 | 1275 | IAE---SFV SLKKFNAFGVH-QDFSFFG--TKI--ERK--R-DRKLNELL NSTIIYQSITGLYESRKRL----- | 1332 |
| WP_033920898 | 1275 | IAE---SFV SLKKFNAFGVH-KDFNFFG--TTI--KRK--R-DRKLKELL NSTIIYQSITGLYESRKRL----- | 1332 |
| AKI42028 | 1278 | IAQ---SFV DLMAFNAMGAP-ASFKFFE--ATI--DRK--R-YTNLKELL SSTIIYQSITGLYESRKRL----- | 1335 |
| AKI50529 | 1278 | IAE---SFV SLKKFNAFGVH-KDFNFFG--TTI--KRK--R-DRKLKELL NSTIIYQSITGLYESRKRI----- | 1335 |
| EFR83390 | 723 | IAQ---SFV DLMVFNAMGAP-ASFKFFE--ATI--DRK--R-YTNLKELL SSTIIYQSITGLYESRKRL----- | 780 |
| WP_046323366 | 1275 | IAQ---SFV NLLEFNAMGAP-ASFKYFE--TNI--ERK--R-YNNLKELL NATIIYQSITGLYEARKRL----- | 1332 |
| AKE81011 | 1322 | -AE---NII HLFTLTNLGAP-AAFKYFD--TTI--DRK--R-YTSTKEVL DATLIHQSITGLYETRIDLSQL-- | 1381 |
| CUO82355 | 1273 | -AN----VI QMLIIMHKGPQnGNIIYDDf--NV-gKRIg-R-LNGRTFYL NIEFISQSPTGIYTKKYKL----- | 1333 |
| WP_033162887 | 1275 | -CD----VI QILIIMHAGPMnGNIMYDDf--KF-tNRIg-R-FTHKNIDL KTTFISTSVTGLFSKKYKL----- | 1335 |
| AGZ01981 | 1339 | -AE---NII HLFTLTNLGAP-AAFKYFD--TTI--DRK--R-YTSTKEVL DATLIHQSITGLYETRIDLSQL-- | 1398 |
| AKA60242 | 1306 | -AE---NII HLFTLTNLGAP-AAFKYFD--TTI--DRK--R-YTSTKEVL DATLIHQSITGLYETRIDLSQL-- | 1365 |
| AKS40380 | 1306 | -AE---NII HLFTLTNLGAP-AAFKYFD--TTI--DRK--R-YTSTKEVL DATLIHQSITGLYETRIDLSQL-- | 1365 |
| 4UN5_B | 1310 | -AE---NII HLFTLTNLGAP-AAFKYFD--TTI--DRK--R-YTSTKEVL DATLIHQSITGLYETRIDLSQL-- | 1369 |
| WP_010922251 | 1366 | GGD | 1368 |
| WP_039695303 | 1370 | GEE | 1372 |
| WP_045635197 | 1368 | GED | 1370 |
| 5AXW_A | | --- | |
| WP_009880683 | 1050 | GGD | 1052 |
| WP_010922251 | 1366 | GGD | 1368 |
| WP_011054416 | 1366 | GGD | 1368 |
| WP_011284745 | 1366 | GGD | 1368 |
| WP_011285506 | 1366 | GGD | 1368 |
| WP_011527619 | 1366 | GGD | 1368 |
| WP_012560673 | 1366 | GGD | 1368 |
| WP_014407541 | 1365 | GGD | 1367 |
| WP_020905136 | 1366 | GGD | 1368 |
| WP_023080005 | 1365 | GGD | 1367 |
| WP_023610282 | 1365 | GGD | 1367 |
| WP_030125963 | 1366 | GGD | 1368 |
| WP_030126706 | 1366 | GGD | 1368 |
| WP_031488318 | 1366 | GGD | 1368 |
| WP_032460140 | 1366 | GGD | 1368 |
| WP_032461047 | 1366 | GGD | 1368 |
| WP_032462016 | 1366 | GGD | 1368 |
| WP_032462936 | 1366 | GGD | 1368 |
| WP_032464890 | 1366 | GGD | 1368 |
| WP_033888930 | 1191 | GGD | 1193 |
| WP_038431314 | 1366 | GGD | 1368 |
| WP_038432938 | 1365 | GGD | 1367 |
| WP_038434062 | 1366 | GGD | 1368 |
| BAQ51233 | 1277 | GGD | 1279 |
| KGE60162 | 541 | GGD | 543 |
| KGE60856 | 304 | GGD | 306 |
| WP_002989955 | 1366 | GGD | 1368 |
| WP_003030002 | 1343 | GED | 1345 |
| WP_003065552 | 1371 | GEE | 1373 |
| WP_001040076 | 1368 | GED | 1370 |
| WP_001040078 | 1376 | GED | 1378 |
| WP_001040080 | 1368 | GED | 1370 |
| WP_001040081 | 1368 | GED | 1370 |
| WP_001040083 | 1368 | GED | 1370 |
| WP_001040085 | 1368 | GED | 1370 |
| WP_001040087 | 1368 | GED | 1370 |
| WP_001040088 | 1368 | GGD | 1370 |
| WP_001040089 | 1368 | GED | 1370 |
| WP_001040090 | 1368 | GED | 1370 |
| WP_001040091 | 1368 | GED | 1370 |
| WP_001040092 | 1368 | GED | 1370 |
| WP_001040094 | 1368 | GED | 1370 |
| WP_001040095 | 1368 | GEG | 1370 |
| WP_001040096 | 1368 | GEG | 1370 |
| WP_001040097 | 1368 | GED | 1370 |
| WP_001040098 | 1368 | GED | 1370 |
| WP_001040099 | 1368 | GED | 1370 |
| WP_001040100 | 1368 | GED | 1370 |
| WP_001040104 | 1368 | GED | 1370 |
| WP_001040105 | 1368 | GED | 1370 |
| WP_001040106 | 1368 | GED | 1370 |
| WP_001040107 | 1368 | GED | 1370 |
| WP_001040108 | 1368 | GED | 1370 |
| WP_001040109 | 1368 | GED | 1370 |
| WP_001040110 | 1368 | GED | 1370 |
| WP_015058523 | 1368 | GED | 1370 |
| WP_017643650 | 1368 | GED | 1370 |
| WP_017647151 | 1368 | GED | 1370 |
| WP_017648376 | 1368 | GED | 1370 |
| WP_017649527 | 1368 | GED | 1370 |
| WP_017771611 | 1368 | GED | 1370 |
| WP_017771984 | 1368 | GED | 1370 |
| CFQ25032 | 1368 | GED | 1370 |
| CFV16040 | 1368 | GED | 1370 |
| KLJ37842 | 1368 | GED | 1370 |
| KLJ72361 | 1368 | GGD | 1370 |
| KLL20707 | 1382 | GED | 1384 |
| KLL42645 | 1368 | GED | 1370 |
| WP_047207273 | 1368 | GED | 1370 |
| WP_047209694 | 1368 | GED | 1370 |
| WP_050198062 | 1368 | GED | 1370 |
| WP_050201642 | 1368 | GED | 1370 |
| WP_050204027 | 1368 | GED | 1370 |
| WP_050881965 | 1368 | GED | 1370 |
| WP_050886065 | 1368 | GED | 1370 |
| AHN30376 | 1368 | GED | 1370 |
| EAO78426 | 1368 | GED | 1370 |
| CCW42055 | 1368 | GED | 1370 |
| WP_003041502 | 1384 | GED | 1386 |
| WP_037593752 | 1344 | GED | 1346 |
| WP_049516684 | 1344 | GED | 1346 |
| GAD46167 | 1343 | GED | 1345 |
| WP_018363470 | 1375 | GEE | 1377 |
| WP_003043819 | 1373 | GGD | 1375 |
| WP_006269658 | 1343 | GED | 1345 |
| WP_048800889 | 1363 | GED | 1365 |
| WP_012767106 | 1369 | GGD | 1371 |
| WP_014612333 | 1369 | GGD | 1371 |
| WP_015017095 | 1369 | GGD | 1371 |
| WP_015057649 | 1369 | GGD | 1371 |
| WP_048327215 | 1369 | GGD | 1371 |
| WP_049519324 | 1369 | GGD | 1371 |
| WP_012515931 | 1346 | GEN | 1348 |
| WP_021320964 | 1346 | GEN | 1348 |
| WP_037581760 | 1346 | GEN | 1348 |
| WP_004232481 | 1375 | GEE | 1377 |
| WP_009854540 | 1368 | GEE | 1370 |
| WP_012962174 | 1369 | GEE | 1371 |
| WP_039695303 | 1370 | GEE | 1372 |
| WP_014334983 | 1373 | GEE | 1375 |
| WP_003099269 | 1366 | GGK | 1368 |
| AHY15608 | | --- | |
| AHY17476 | | --- | |
| ESR09100 | 198 | GGK | 200 |
| AGM98575 | | --- | |
| ALF27331 | 1343 | GGD | 1345 |
| WP_018372492 | 1351 | GEN | 1353 |
| WP_045618028 | 1374 | GED | 1376 |
| WP_045635197 | 1368 | GED | 1370 |
| WP_002263549 | 1343 | GGD | 1345 |
| WP_002263887 | 1343 | GGD | 1345 |
| WP_002264920 | 1343 | GGD | 1345 |
| WP_002269043 | 1343 | GGD | 1345 |
| WP_002269448 | 1343 | GGD | 1345 |
| WP_002271977 | 1343 | GGD | 1345 |
| WP_002272766 | 1343 | GGD | 1345 |
| WP_002273241 | 1343 | GGD | 1345 |
| WP_002275430 | 1343 | GGD | 1345 |
| WP_002276448 | 1343 | GGD | 1345 |
| WP_002277050 | 1353 | GGD | 1355 |
| WP_002277364 | 1343 | GGD | 1345 |
| WP_002279025 | 1343 | GGD | 1345 |
| WP_002279859 | 1343 | GGD | 1345 |
| WP_002280230 | 1343 | GGD | 1345 |
| WP_002281696 | 1343 | GGD | 1345 |
| WP_002282247 | 1353 | GGD | 1355 |
| WP_002282906 | 1343 | GGD | 1345 |
| WP_002283846 | 1343 | GGD | 1345 |
| WP_002287255 | 1343 | GGD | 1345 |
| WP_002288990 | 1343 | GGD | 1345 |
| WP_002289641 | 1343 | GGD | 1345 |
| WP_002290427 | 1343 | GGD | 1345 |
| WP_002295753 | 1343 | GGD | 1345 |
| WP_002296423 | 1343 | GGD | 1345 |
| WP_002304487 | 1357 | GGD | 1359 |
| WP_002305844 | 1343 | GGD | 1345 |
| WP_002307203 | 1343 | GGD | 1345 |
| WP_002310390 | 1343 | GGD | 1345 |
| WP_002352408 | 1343 | GGD | 1345 |
| WP_012997688 | 1343 | GGD | 1345 |
| WP_014677909 | 1343 | GGD | 1345 |
| WP_019312892 | 1343 | GGD | 1345 |
| WP_019313659 | 1343 | GGD | 1345 |
| WP_019314093 | 1343 | GGD | 1345 |
| WP_019315370 | 1343 | GGD | 1345 |
| WP_019803776 | 1343 | GGD | 1345 |
| WP_019805234 | 1343 | GGD | 1345 |
| WP_024783594 | 1343 | GGD | 1345 |
| WP_024784288 | 1353 | GGD | 1355 |
| WP_024784666 | 1343 | GGD | 1345 |
| WP_024784894 | 1343 | GGD | 1345 |
| WP_024786433 | 1353 | GGD | 1355 |
| WP_049473442 | 1343 | GGD | 1345 |
| WP_049474547 | 1343 | GGD | 1345 |
| EMC03581 | 1336 | GGD | 1338 |
| WP_000428612 | 1371 | GED | 1373 |
| WP_000428613 | 1369 | GED | 1371 |
| WP_049523028 | 1364 | GEE | 1366 |
| WP_003107102 | 1335 | GGD | 1337 |
| WP_054279288 | 1366 | GGD | 1368 |
| WP_049531101 | 1374 | GED | 1376 |
| WP_049538452 | 1374 | GED | 1376 |
| WP_049549711 | 1376 | GED | 1378 |
| WP_007896501 | 1372 | GGD | 1374 |
| EFR44625 | 1324 | GGD | 1326 |
| WP_002897477 | 1368 | GEE | 1370 |
| WP_002906454 | 1375 | GED | 1377 |
| WP_009729476 | 1369 | GED | 1371 |
| CQR24647 | 1359 | GGE | 1361 |
| WP_000066813 | 1373 | GED | 1375 |
| WP_009754323 | 1369 | GED | 1371 |
| WP_044674937 | 1362 | GGD | 1364 |
| WP_044676715 | 1364 | GGD | 1366 |
| WP_044680361 | 1364 | GGD | 1366 |
| WP_044681799 | 1362 | GGD | 1364 |
| WP_049533112 | 1384 | GED | 1386 |
| WP_029090905 | | --- | |
| WP_006506696 | | --- | |
| AIT42264 | 1366 | GGD | 1389 |
| WP_034440723 | | --- | |
| AKQ21048 | 1366 | GGD | 1384 |
| WP_004636532 | 1330 | -ED | 1332 |
| WP_002364836 | 1336 | -VD | 1337 |
| WP_016631044 | 1287 | -VD | 1288 |
| EMS75795 | 1077 | GEQ | 1079 |
| WP_002373311 | 1336 | -VD | 1337 |
| WP_002378009 | 1336 | -VD | 1337 |
| WP_002407324 | 1336 | -VD | 1337 |
| WP_002413717 | 1336 | -VD | 1337 |
| WP_010775580 | 1338 | -VD | 1339 |
| WP_010818269 | 1336 | -VD | 1337 |
| WP_010824395 | 1336 | -VD | 1337 |
| WP_016622645 | 1336 | -VD | 1337 |
| WP_033624816 | 1336 | -VD | 1337 |
| WP_033625576 | 1336 | -VD | 1337 |
| WP_033789179 | 1336 | -VD | 1337 |
| WP_002310644 | | --- | |
| WP_002312694 | | --- | |
| WP_002314015 | | --- | |
| WP_002320716 | | --- | |
| WP_002330729 | | --- | |
| WP_002335161 | | --- | |
| WP_002345439 | | --- | |
| WP_034867970 | 1334 | GEQ | 1336 |
| WP_047937432 | | --- | |
| WP_010720994 | 1334 | GEQ | 1336 |
| WP_010737004 | 1334 | GEQ | 1336 |
| WP_034700478 | 1334 | GEQ | 1336 |
| WP_007209003 | | --- | |
| WP_023519017 | 1327 | GER | 1330 |
| WP_010770040 | 1333 | -VD | 1334 |
| WP_048604708 | 1329 | -GD | 1330 |
| WP_010750235 | 1337 | GVQ | 1339 |
| AII16583 | 1405 | GGD | 1424 |
| WP_029073316 | | --- | |
| WP_031589969 | | --- | |
| KDA45870 | | --- | |
| WP_039099354 | | --- | |
| AKP02966 | | --- | |
| WP_010991369 | 1333 | -DD | 1334 |
| WP_033838504 | 1333 | -DD | 1334 |
| EHN60060 | 1336 | -DD | 1337 |
| EFR89594 | 1102 | -DD | 1103 |
| WP_038409211 | 1333 | -ED | 1334 |
| EFR95520 | 952 | -ED | 953 |
| WP_003723650 | 1333 | -DD | 1334 |
| WP_003727705 | 1333 | -DD | 1334 |
| WP_003730785 | 1333 | -DD | 1334 |
| WP_003733029 | 1333 | -DN | 1334 |
| WP_003739838 | 1333 | -DG | 1334 |
| WP_014601172 | 1333 | -DD | 1334 |
| WP_023548323 | 1333 | -DS | 1334 |
| WP_031665337 | 1333 | -DD | 1334 |
| WP_031669209 | 1333 | -DN | 1334 |
| WP_033920898 | 1333 | -DS | 1334 |
| AKI42028 | 1336 | -DD | 1337 |
| AKI50529 | 1336 | -DS | 1337 |
| EFR83390 | 781 | -DD | 782 |
| WP_046323366 | 1333 | -DD | 1334 |
| AKE81011 | 1382 | GGD | 1400 |
| CUO82355 | | --- | |
| WP_033162887 | | --- | |
| AGZ01981 | 1399 | GGD | 1417 |
| AKA60242 | 1366 | GGD | 1368 |
| AKS40380 | 1366 | GGD | 1376 |
| 4UN5_B | 1370 | GGD | 1372 |

Additional suitable Cas9 sequences in which amino acid residues homologous to residues 10, 122, 137, 182, 262, 294, 409, 480, 543, 660, 694, 840, 1219, and/or 1329 of SEQ ID NO: 9 can be identified are known to those of skill in the art. See, e.g., Supplementary Table S2 and Supplementary Figure S2 of Fonfara et al., Phylogeny of Cas9 determines functional exchangeability of dual-RNA and Cas9 among orthologous type II CRISPR-Cas systems, Nucl. Acids Res. 2013, doi: 10.1093/nar/gkt1074, which are incorporated herein by reference in their entirety. Cas9 variants of the sequences provided herein or known in the art comprising one or more mutations, e.g., at least one, at least two, at least three, at least four, at least five, at least six, or at least seven mutations as provided herein, e.g., of one or more amino acid residue that is homologous to amino acid residue 10, 122, 137, 182, 262, 294, 409, 480, 543, 660, 694, 840, 1219, and/or 1329 in SEQ ID NO: 9 are provided by this disclosure, for example, Cas9 variants comprising a A262T, K294R, S409I, E480K, E543D, M694I, and/or E1219V mutation.

### Example 4: Evolution of Cas9 with Broadened PAM Specificities

By evolving S. pyogenes Cas9 on a NNN PAM library using PACE, Cas9s with broadened PAM specificities have been evolved with higher activities for many non-canonical PAMs. Such a Cas9 still retains its native DNA binding and cutting activity and can be utilized with all of the current tools available. It has been hypothesized that by modulating Cas9's interactions with the DNA, the PAM specificity of Cas9 can be modified and expanded. Other Cas9s such as Staphylococcus aureus could also be engineered to change and expand their PAM specificity by such a method. Methods to modulate DNA binding such as targeted mutagenesis of the Cas9 protein, fusions to DNA-binding proteins, and the use of multiple Cas9 proteins tethered to each other could also expand the PAMs that can be targeted.

Cas9 Evolution. After evolution using overnight propagation of the phage with the mutagenesis plasmid (MP), the resultant phages containing mutations, discussed above, in PACE were used. Twenty-four individual phages were sequenced from the PACE run. Mutations found in the Cas9 gene are documented in Table 3 below. The Cas9 gene containing these mutations was cloned out of the phage and into plasmids to test both DNA binding and cutting activity.

TABLE 3
Cas9 mutations

| | | | | | | | | | | | | |
|---|---|---|---|---|---|---|---|---|---|---|---|---|
| pJH306 (WT | | | | | | | | | | | | |
| dCas9-VPR) | | | | | | | | | | | | |
| pJH407 | | | | | | | | | | | | |
| (WT Cas9) | | | | | | | | | | | | |
| pJH562 | | | | | | E480K | E543D | | E1219V | | | |
| (dCas9-VPR) | | | | | | | | | | | | |
| pJH599 | A262T | | | | S409I | E480K | E543D | M694I | E1219V | | | |
| (dCas9-VPR) | | | | | | | | | | | | |
| pJH600 | | | K294R | | | E480K | E543D | | E1219V | | Q1256K | L1362P |
| (dCas9-VPR) | | | | | | | | | | | | |
| pJH601 | A262T | | | | S409I | E480K | E543D | M694I | E1219V | | | |
| (dCas9-VPR) | | | | | | | | | | | | |
| pJH602 | | | K294R | | | E480K | E543D | | E1219V | | Q1256K | |
| (dCas9-VPR) | | | | | | | | | | | | |
| pJH603 | A262T | | | | S409I | E480K | E543D | M694I | E1219V | | | |
| (dCas9-VPR) | | | | | | | | | | | | |
| pJH604 | | S267G | K294R | | | E480K | E543D | | E1219V | N1224K | Q1256K | |
| (dCas9-VPR) | | | | | | | | | | | | |
| pJH605 | A262T | | | F405I | S409I | E480K | E543D | M694I | E1219V | | | |
| (dCas9-VPR) | | | | | | | | | | | | |
| pJH760 (pJH599 | A262T | | | | S409I | E480K | E543D | M694I | E1219V | | | |
| Minus VPR, | | | | | | | | | | | | |
| Nuclease | | | | | | | | | | | | |
| Positive) | | | | | | | | | | | | |

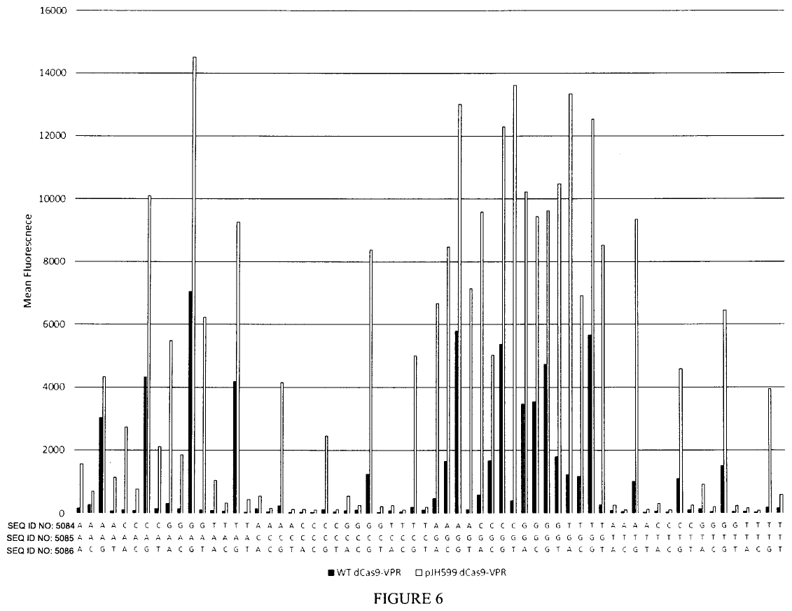

GFP Activation in Human Cell Culture. Testing was performed on a reporter with a GFP reporter activated by dCas9-VPR. Testing was performed on 5′-NGG-3′ PAM first (FIGS. 4A and 4B) and then with a library of GFP reporters all containing the same Cas9 target site, but with NNN at the PAM position (FIGS. 5A and 5B). In this testing, dCas9-VPR was found to activate the GFP signal. Wild-type Cas9 with no mutations (pJH306), Cas9 evolved from overnight propagation (pJH562), and Cas9 evolved from PACE (pJH599-pJH605) were all tested. Mutations for each Cas9 are documented in Table 3 above.

dCas9-VPR on all 64 PAM Sequences. pJH306 (WT dCas9-VPR) and pJH599 (WT dCas9-VPR with mutations A262T, S409I, E480K, E543D, M694I, and E1219V) were tested on all 64 PAM sequences (FIG. 6). dCas9-VPR was used to activate GFP as before and a different reporter plasmid was used for each well to ascertain the activation efficiency for all 64 different PAM sequences. The mean GFP fluorescence was measured for all transfected cells, as gated by iRFP signal. pJH599 showed either improved or similar levels of activation as compared to pJH306 for all PAM sequences.

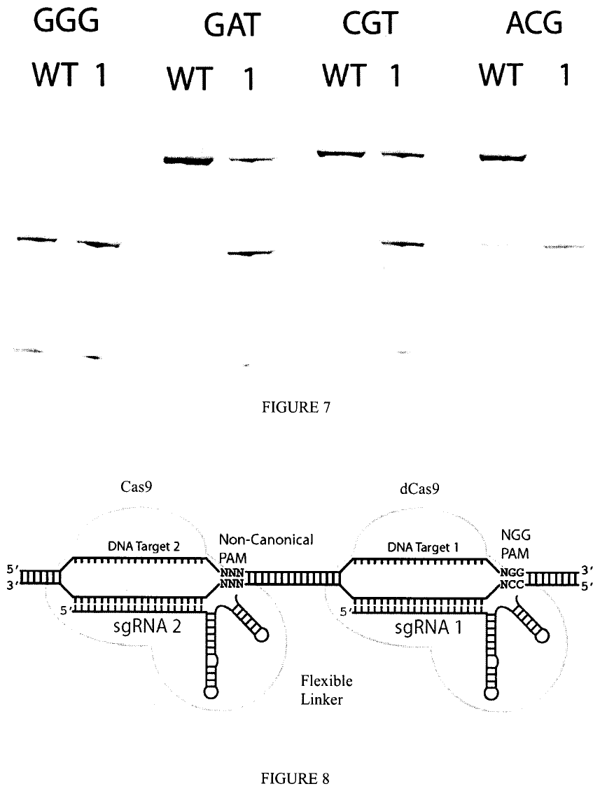

In Vitro Cutting Assay. The ability of expressed and purified WT Cas9 (WT) and Cas 9 with an E1219V mutation (1) to cut DNA with different PAMs was tested (FIG. 7). Cas9 was incubated with dsDNA containing the target site. Cutting was measured by running the DNA on a gel to compare the amount of uncut product to the amount of cut products, which run faster due to their smaller size. The E1219V mutation was found to increase Cas9 cutting activity on non-canonical PAMs while maintaining activity on 5′-NGG-3′ PAMs.

Evolving Different Systems. In addition to S. pyogenes Cas9 evolution, other Cas9 systems such as S. aureus, S. thermophilus, N. meningitidis, and T. denticola, etc. can be evolved to modify and expand their PAM specificities. The data indicates that, by using a similar system to the S. pyogenes Cas9 evolution, a phage containing the S. aureus Cas9 can also be evolved to expand its PAM specificity.

Modulating PAM Specificity. By mutating neutral and negatively charged amino acids to positively charged amino acids, Cas9 could be modified to expand the PAMs that can be targeted. Generally, incorporating mutations into Cas9 proteins that generate a net increase in positive charge may increase the affinity of Cas9 to bind DNA. In combination with the Cas9 mutations provided herein, additional residues that could be mutated for increased PAM targeting in S. pyogenes Cas9 further include ones that have been identified to change the PAM specificity (D1135, G1218, R1333, R1335, T1337) 38 and residues that can increase Cas9 activity (S845 and L847) 37. Residues that increase Cas9 specificity such as the mutations of arginine, histidine, and lysine to alanine previously identified³⁷ and the mutations of asparagine, arginine, and glutamine to alanine as previously identified³⁹ could lead to lower tolerability for non-canonical PAMs as these mutations presumably decrease the interactions between the Cas9 and DNA.

Fusions to Modulate PAM Specificity. Programmable DNA-binding proteins such as zinc-finger domains, TALEs, and other Cas9 proteins could be fused to Cas9 to improve the ability to target nucleotide sequences having canonical or non-canonical PAMs, for example to increase activity, specificity or efficiency. A nuclease-null dCas9 could be fused to a nuclease active Cas9 to increase the ability of the nuclease active Cas9 to target different PAM sequences. One example of a nuclease-null dCas9 fused to a nuclease active Cas9 is shown in FIG. 8. Such fusions can be useful for improving the ability to target nucleotide sequences having canonical or non-canonical PAMs. The Cas9s could either be from the same species or from different species. Furthermore, both Cas9s could be nuclease-null dCas9, and could be further fused to effector proteins such as VP64, VP64-p65-Rta, FokI, GFP and other fluorescent proteins, deaminases, or any of the effector proteins provided herein.

Using Cas9 to Localize Other Nucleases and Other DNA-Binding Proteins. Cas9 can also be used to overcome the native binding specificity of other proteins by localizing them to their DNA targets. DNA nucleases, recombinases, deaminases, and other effectors often have a native DNA specificity. Cas9 can be fused to these proteins to overcome and expand their native DNA specificities. gRNAs will target the Cas9 adjacent to the DNA effector's target site and will help to localize them to their target site.

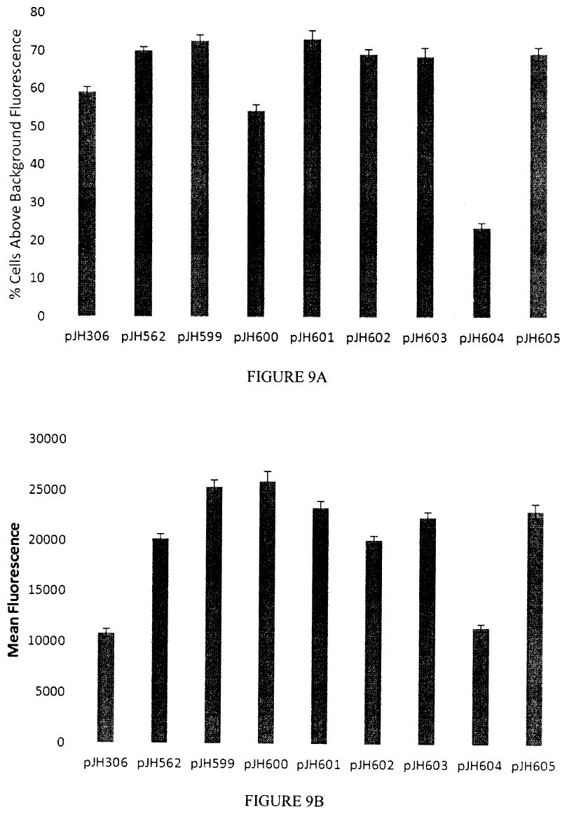

dCas9-VPR on NNNNN PAM Library. To test that the evolved Cas9s had not picked up specificity in the 4th and 5th PAM positions, dCas9-VPR on a NNNNN PAM library was tested. As seen with the NNN library, most of the constructs (e.g., pJH562, pJ559, pJH600, pJH601, pJH602, pJH603, and pJH605) showed improved activity. pJH599 consistently showed improvement in both the percentage of cells that showed GFP activation (FIG. 9A) and the mean fluorescence of those cells that showed GFP activation (FIG. 9B).

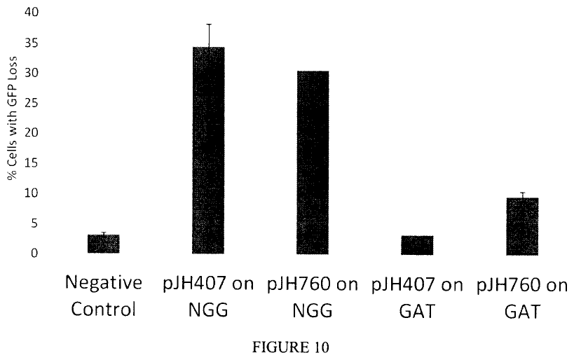

Cas9 GFP Cutting. The WT Cas9, pJH407, was compared with a nuclease-positive evolved Cas9, pJH760 (FIG. 10). pJH760 contained the same mutations as pJH599 but without the D10A and H480A nuclease-inactivating mutations, and without the VPR fusion. A genomically integrated GFP gene was cut by Cas9 and the activity was measured by the loss of GFP signal in cells. On a site with 5′-NGG-3′ PAM, pJH407 and pJH760 showed comparable activity. On a site with GAT PAM, pJH760 showed a significant increase in activity as compared to pJH407.

### Example 5 Cas9:DNA Editing Enzyme Fusion Proteins

This disclosure further provides Cas9 fused with a DNA editing enzyme for the targeted editing of DNA sequences. FIG. 11 illustrates double stranded DNA substrate binding by DNA editing enzyme-dCas9:sgRNA complexes. The DNA editing enzyme shown is a deaminase. The structures according to FIG. 11 are identified in these sequences (36 bp: underlined, sgRNA target sequence: bold; PAM: boxed; 21 bp: italicized).

### Example 6 PAM Depletion Assay

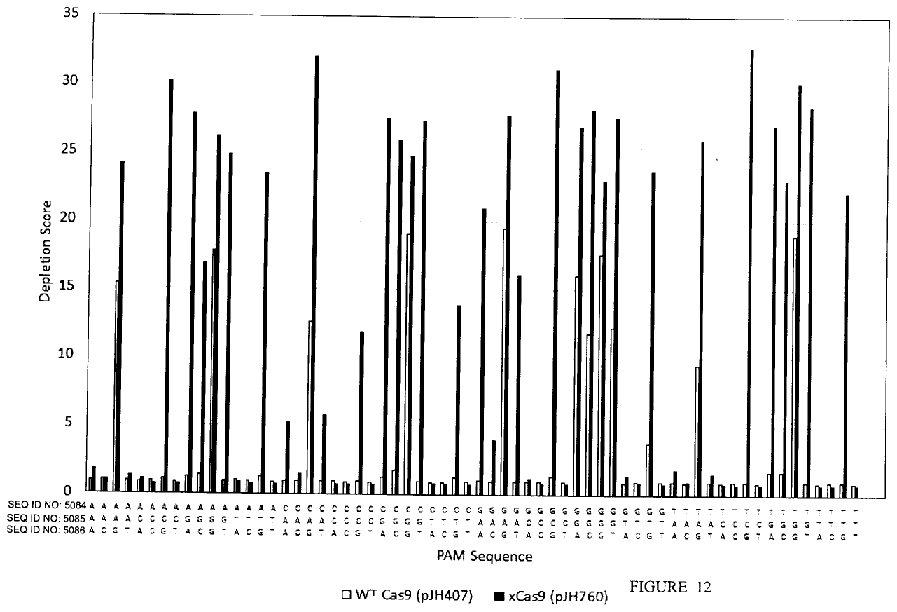

In E. coli, a library of PAM sequences are encoded in a plasmid that also contains an antibiotic gene. If the Cas9 can cut the PAM sequences on the plasmid, the plasmid is not replicated and is lost; only plasmids that are uncut remain the population. The plasmids that were cut by the Cas9 can be determined by sequencing the initial plasmid population and the final plasmid population through high throughput sequencing. The proportion of the library consisting of each PAM sequence was obtained by dividing the number of reads containing the PAM sequence by the total number of reads. The depletion score was then calculated by dividing the proportion of the library containing the PAM section before the selection by the proportion of the library containing the PAM sequence after selection. Higher depletion scores signify higher cutting activity by the Cas9 of that particular PAM sequence. The results of the PAM depletion assay are shown in FIG. 12.

A number of PAM sequences that were not cut with the wild-type Cas9 were cut with the evolved Cas9 (xCas9 v1.0, pJH760). Notably, all PAM sequences of the form NGN and NNG, as well as GAA and GAT showed a depletion greater than 10-fold with the xCas9. A single G in either the second or third PAM position could be sufficient for cutting with the newly evolved Cas9, opening up the sequence space significantly for the target sites that can be targeted using Cas9. The PAM depletion scores are given in Table 4.

TABLE 4
PAM Depletion Scores

| PAM Sequence | WT Cas9 | xCas9 v1.0 |
|---|---|---|
| TCG | 0.96 | 32.75 |
| CAG | 12.63 | 32.01 |
| GCG | 1.38 | 31.14 |
| ACG | 1.12 | 30.15 |
| TGG | 18.99 | 30.13 |
| TGT | 1.01 | 28.37 |
| GGC | 11.78 | 28.17 |
| AGA | 1.25 | 27.80 |
| GAG | 19.47 | 27.71 |
| GGT | 12.22 | 27.59 |
| CGA | 1.27 | 27.53 |
| CGT | 0.97 | 27.30 |
| TGA | 1.72 | 27.02 |
| GGA | 16.03 | 26.90 |
| AGG | 17.80 | 26.19 |
| TAG | 9.54 | 25.98 |
| CGC | 1.76 | 25.88 |
| AGT | 0.97 | 24.88 |
| CGG | 19.03 | 24.77 |
| AAG | 15.40 | 24.16 |
| GTG | 3.76 | 23.71 |
| ATG | 1.26 | 23.48 |
| GGG | 17.59 | 23.01 |
| TGC | 1.71 | 23.00 |
| TTG | 0.98 | 22.15 |
| GAA | 1.04 | 20.96 |
| AGC | 1.43 | 16.85 |
| GAT | 1.02 | 16.09 |
| CTG | 1.29 | 13.88 |
| CCG | 1.02 | 11.86 |
| CAT | 0.99 | 5.75 |
| CAA | 1.00 | 5.28 |
| GAC | 0.98 | 3.96 |
| TAA | 0.97 | 1.90 |
| AAA | 1.02 | 1.81 |
| TAT | 0.95 | 1.56 |
| CAC | 1.01 | 1.48 |
| GTA | 0.94 | 1.42 |
| AAT | 0.98 | 1.39 |
| GCA | 0.98 | 1.22 |
| ACA | 0.95 | 1.13 |
| AAC | 1.08 | 1.09 |
| TAC | 0.92 | 0.95 |
| ATA | 1.04 | 0.94 |
| GTC | 0.99 | 0.92 |
| GCT | 0.99 | 0.87 |
| TCA | 0.94 | 0.84 |
| CTA | 0.95 | 0.83 |
| GCC | 0.99 | 0.82 |
| GTT | 0.97 | 0.81 |
| TCC | 0.98 | 0.80 |
| ACC | 0.96 | 0.80 |
| CTC | 0.92 | 0.80 |
| CCA | 0.97 | 0.79 |
| CCC | 0.95 | 0.79 |
| CTT | 0.96 | 0.79 |
| ATC | 0.96 | 0.78 |
| CCT | 0.94 | 0.78 |
| ATT | 0.93 | 0.77 |
| ACT | 0.89 | 0.77 |
| TTT | 0.94 | 0.77 |
| TCT | 0.95 | 0.77 |
| TTC | 0.96 | 0.76 |
| TTA | 0.94 | 0.76 |

### Example 7: GFP Cutting Mammalian Cells

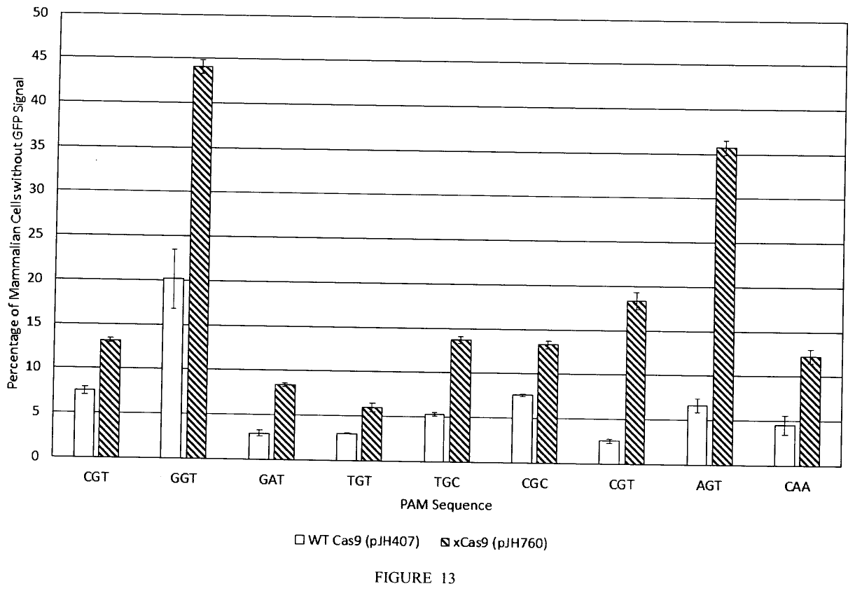

Wild-type or evolved Cas9 and gRNAs were transfected into mammalian cells containing a genomically integrated GFP gene. Different gRNAs targeted different sites with different PAM sequences, such that cutting of the GFP by Cas9 would lead to a loss of the GFP signal. The GFP signal was quantified after five days with flow cytometry. As shown in FIG. 13, evolved Cas9 cut GFP in mammalian, in line with the results in the GFP activation assay and in the PAM depletion assay. High throughput sequencing around the cut site verified the results seen with the flow cytometer; the indels were proportional to the cutting percentage seen with the flow cytometer.

### Example 8: Further Evaluation of the HHH Library

As SpCas9 has a preference for the G residues at the second and third base, evaluation was continued using the end point from the last evolution on a HHH (H=A, C, or T) PAM library. After evolution, 13 colonies were sequenced and a number of new mutations were identified. Three mutations, the E1219V, E480K, and E543D mutations were found in all the clones. A number of the clones either had the S267G/K294R/Q1256K mutations or the A262T/S409I mutations but those mutations were never seen together, suggesting that the clones had taken two divergent paths along the evolutionary landscape. The new mutations are given in Table 5.

TABLE 5
New Mutations

| HHH | HHH | HHH | HHH | HHH | HHH | HHH | HHH | HHH | HHH | HHH | HHH | HHH |
|---|---|---|---|---|---|---|---|---|---|---|---|---|
| #1 | #2 | #3 | #4 | #5 | #6 | #7 | #8 | #9 | #10 | #11 | #12 | #13 |
| (xCas9 | (xCas9 | (xCas9 | (xCas9 | (xCas9 | (xCas9 | (xCas9 | (xCas9 | (xCas9 | (xCas9 | (xCas9 | (xCas9 | (xCas9 |
| 2.0) | 2.1) | 2.2) | 2.3) | 2.4) | 2.5) | 2.6) | 2.7) | 2.8) | 2.9) | 2.10) | 2.11) | 2.12) |
| | | D23N | | | | | | | | | | |
| | | E108G | | | | | | | | | | |
| R115H | | | | | | | | | | | | |
| K141Q | | | | | | | | | | | | |
| | | | | | | | | | D180N | | | |
| | | | | | | | | P230S | | | | P230S |
| | | | D257N | | | | | | | | | |
| | | A262T | | | | A262T | | | | A262T | | |
| S267G | S267G | | S267G | | S267G | | | S267G | | | S267G | S267G |
| | | | | | | | | | D284N | | | |
| K294R | K294R | | K294R | | K294R | | K294R | K294R | | | K294R | K294R |
| | | | | | | R324L | | | | | | |
| Q394H | | | | | | | | | | | | |
| | | S409I | | S409I | | S409I | | | | S409I | | |
| | | | | LA55F | | | | | | | | |
| | | | T466A | | | | | | | | | |
| | | | | | | | | | T474I | | | |
| E480K | E480K | E480K | E480K | E480K | E480K | E480K | E480K | E480K | E480K | E480K | E480K | E480K |
| E543D | E543D | E543D | E543D | E543D | E543D | E543D | E543D | E543D | E543D | E543D | E543D | E543D |
| | | | | | | | | | K554R | | | |
| | | | | R654L | | | | | | | | |
| | | M694I | | | | M694I | | | | M694I | | |
| | | | | | | | A711E | | | | | |
| | | L727P | | | | | | | | | | |
| | | | | | | | | | M763I | | | |
| | | | I1063V | | | | | | | | | |
| | | | | V1100I | | | | | | | | |
| E1219V | E1219V | E1219V | E1219V | E1219V | E1219V | E1219V | E1219V | E1219V | E1219V | E1219V | E1219V | E1219V |
| | | | | | | | | | K1244N | | | |
| Q1256K | Q1256K | | Q1256K | | Q1256K | | Q1256K | Q1256K | | | Q1256K | Q1256K |
| | | K1289Q | | | | | | | | | | |
| | | | | A1323S | | | | | | | | |

### Example 9: Further Evolved Cas9 Proteins for Gene Editing

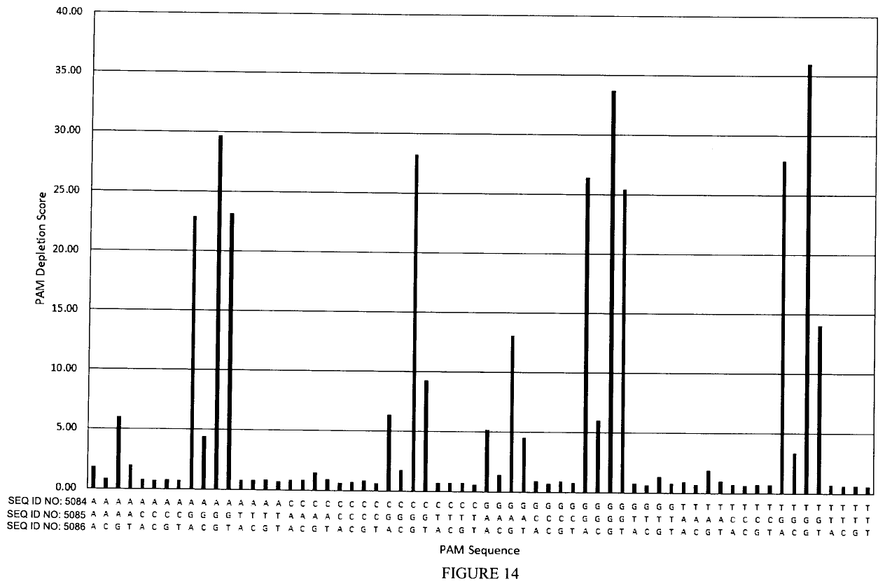

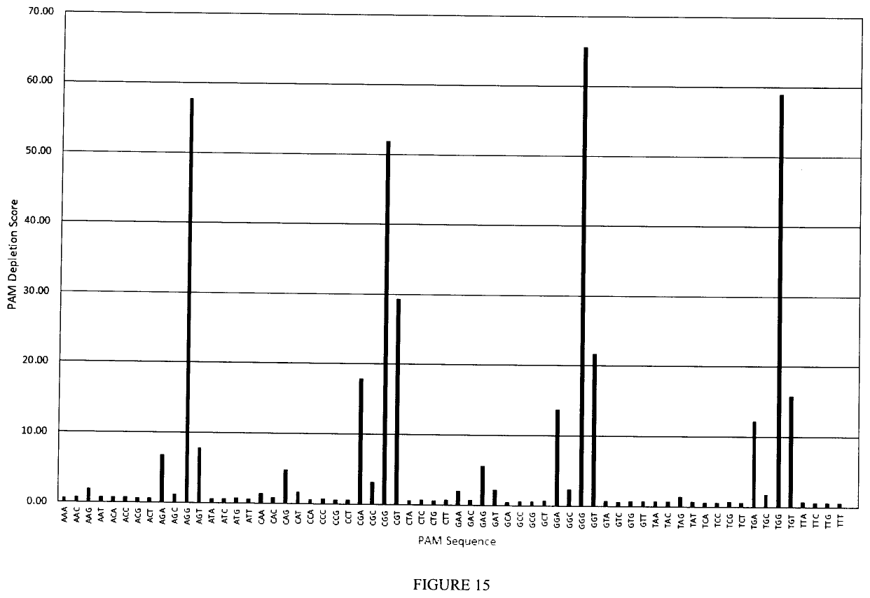

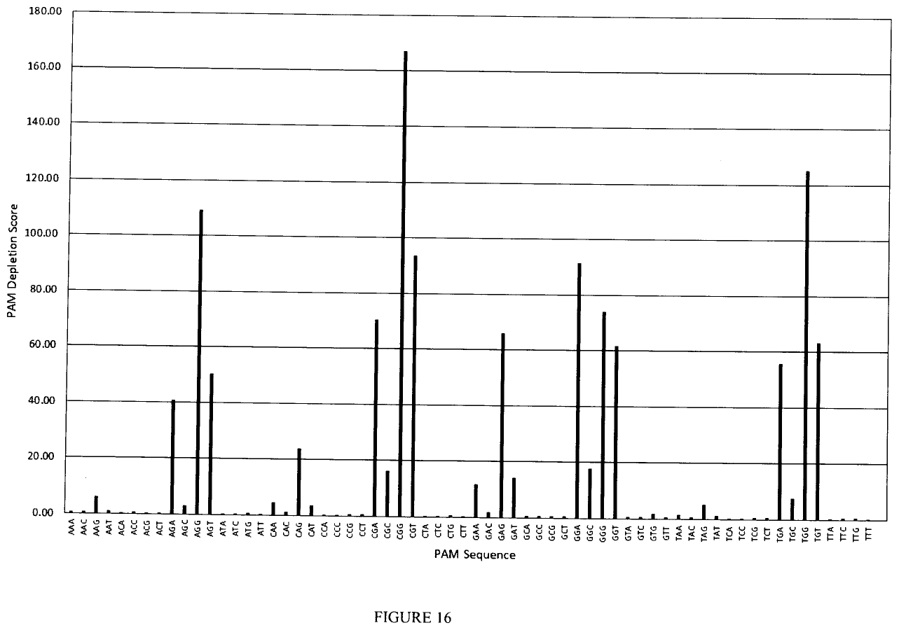

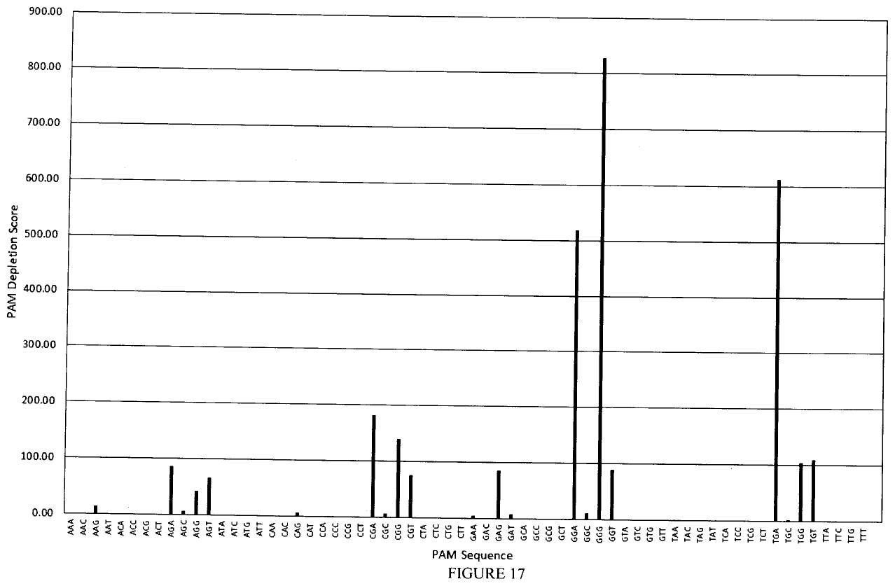

pJH760, described in Example 6, was tested in the PAM depletion assay on a number of new targets. Four new targets were selected: re2 (GGGGCCACTAGGGACAGGAT (SEQ ID NO: 314)), a synthetic target previously used for GFP activation in mammalian cells; VEGF (GGGTGGGGGGAGTTTGCTCC (SEQ ID NO: 315)), a target within the VEGF gene, CLTA (GCAGATGTAGTGTTTCCACA (SEQ ID NO: 316)), a target within the CLTA gene; and CCR5D (TCACTATGCTGCCGCCCAGT (SEQ ID NO: 317)), a target within the CCR5D gene. The results of the PAM depletion assay are given in FIGS. 14 to 17. It was found that, in addition to the canonical NGG sequence, PAM depletion demonstrates cutting for most NGN and some NNG sequences as well.

The HHH PAM library was further evolved using the end point from the last evolution on a HHH (H=A, C, or T) PAM library. After evolution, 13 colonies were sequenced and a number of new mutations were identified. Three mutations, the E1219V, E480K, and E543D mutations were found in all the clones. A number of the clones either had the K294R/Q1256K mutations or the A262T/S409I mutations but those mutations were never seen together, suggesting that the clones had taken two divergent paths along the evolutionary landscape. The new mutations are given in Tables 8 and 9 below.

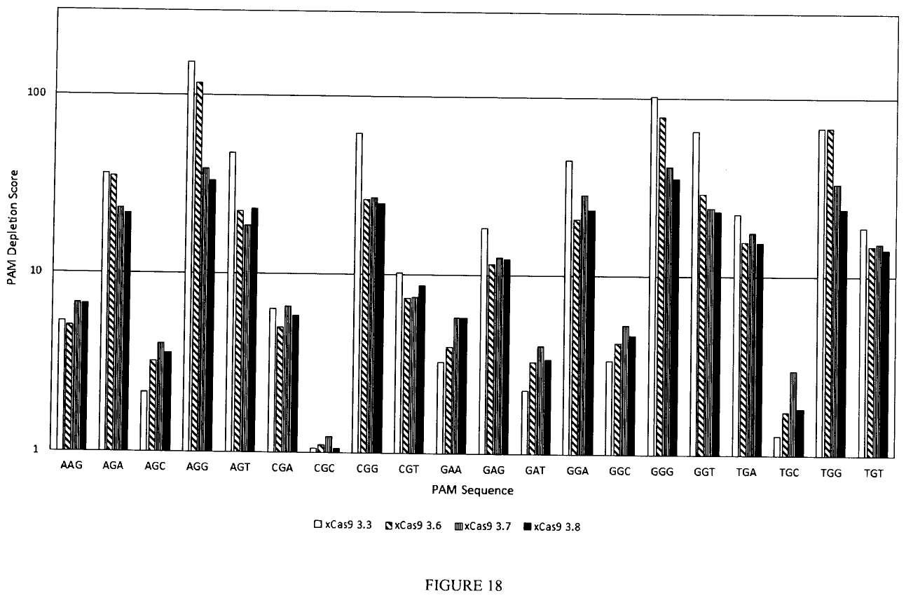

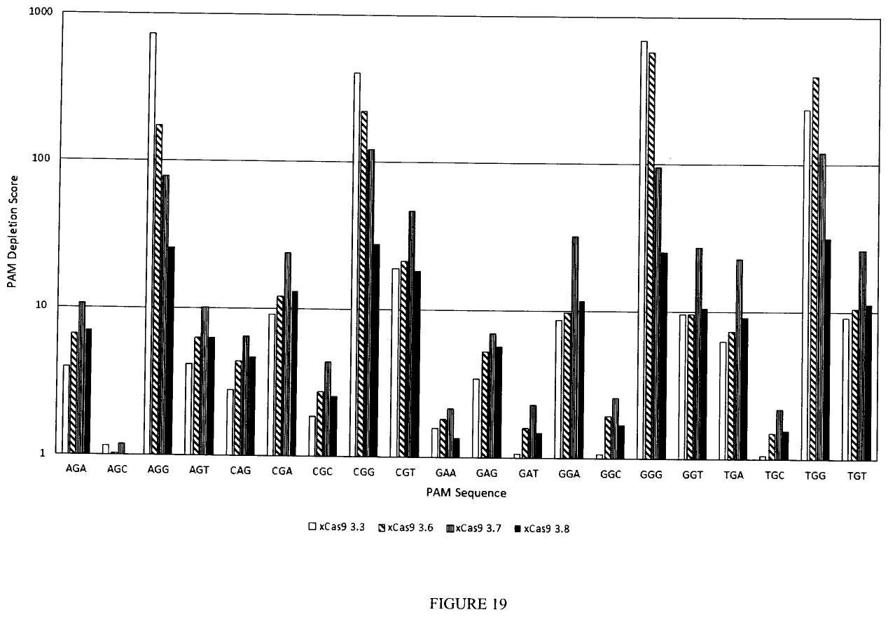

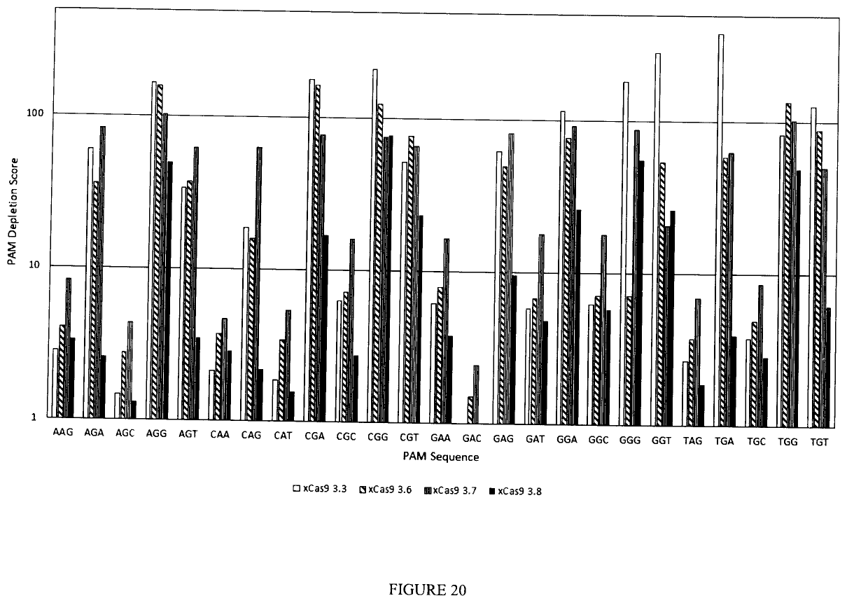

As expected, variation in activity was seen with different targets. The PAM depletion assay scores are given in Table 10. NGN consistently showed cutting activity with some targets. Variation was seen amongst the xCas9 3.x mutants in terms of which mutant had the best activity. Of note, xCas9 3.3 contained the K294R/Q1256K series of mutations, while the other three mutants (3.6, 3.7, and 3.8) contained the A262T/S409I series of mutations. xCas9 3.6 and 3.7 outperformed 3.8. While 3.3 seemed to have the highest activity for most cases, 3.6 and 3.7 performed better on certain PAM sequences. The results of the PAM depletion assays for three of the new targets described above are given in FIGS. 18 to 20.

A NNNNN PAM depletion library was constructed. It was assayed to examine any fourth or fifth base specificity. Initial results of the PAM depletion assay show that there is no preference at the fourth and fifth base as expected.

In summary, E1219V was found to be one of the earliest mutations that fixed in the evaluation. It is close to the PAM sequence in the crystal structure. E480K and E543D were also seen in all of the clones from the early stages of the evolution, and may be important. K294R/Q1256K and A262T/S409I seem to be two divergent paths, and may be important. Their PAM sequence profiles seem to be slightly different, which implies their importance relative to PAM activity determination.

TABLE 6
Diseases/disorders containging T to C Changes. The table includes human gene
mutations that may be corrected by changing a cytosine (C) to a thymine (T). The gene
name, gene symbol,and Gene ID are indicated.

| SEQ | | | | |
|---|---|---|---|---|
| ID | | Gene | Gene | |
| NO: | Name | ID | Symbol | Flanks |
| 674 | NM_000410.3(HFE): c.314T > C | 3077 | HFE | [′ATGTTCACTGTTGACTTCTGGACTAYTATGG |
| | (p.Ile105Thr) | | | AAAATCACAACCACAGCAAG′] |
| 675 | NM_144631.5(ZNF513): | 130557 | ZNF513 | [′GCTGGGAGCTGCCATGTGTGGGCGCYGCAT |
| | c.1015T > C (p.Cys339Arg) | | | GCGAGGAGAGGCTGGAGGGGG′] |
| 676 | NM_001199107.1(TBC1D24): | 57465 | TBC1D24 | [′CGTGGCGCTGGCCATCCTCAAGTTCYTCCAC |
| | c.751T > C (p.Phe251Leu) | | | AAGGTGAGGGCCGGGCAGCC′] |
| 677 | NM_001708.2(OPN1SW): | 611 | OPN1SW | [′CATCTTCTGCTTCATTGTGCCTCTCBCCCTCA |
| | c.640T > C (p.Ser214Pro) | | | TCTGCTTCTCCTACACTCA′] |
| 678 | NM_000235.3(LIPA): c.599T > C | 3988 | LIPA | [′AAAAGGATTAAAATGTTTTTTGCCCYGGGTC |
| | (p.Leu200Pro) | | | CTGTGGCTTCCGTCGCCTTC′] |
| 679 | NM_000071.2(CBS): c.833T > C | 875 | CBS | [′CTGAAGCCGCGCCCTCTGCAGATCAYTGGG |
| | (p.Ile278Thr) | | | GTGGATCCCGAAGGGTCCATC′] |
| 680 | NM_000071.2(CBS): c.1616T > C | 875 | CBS | [′TTCGGGGTGGTCACCGCCATTGACTYGCTGA |
| | (p.Leu539Ser) | | | ACTTCGTGGCCGCCCAGGAG′] |
| 681 | NM_033409.3(SLC52A3): | 113278 | SLC52A3 | [′CGCCCACTTCTCACCCCTGGTCTTCYTCCTCC |
| | c.670T > C (p.Phe224Leu) | | | TCCTATCCATCATGATGGC′] |
| 682 | NM_033409.3(SLC52A3): | 113278 | SLC52A3 | [′CTTTTCAGCGGCTGCCTCAGTTACGYCAAGG |
| | c.1238T > C (p.Val413Ala) | | | TGATGCTGGGCGTGGTCCTG′] |
| 683 | NM_000274.3(OAT): c.163T > C | 4942 | OAT | [′GGAATATAAGTATGGTGCACACAACYACCA |
| | (p.Tyr55His) | | | TCCTTTACCTGTAGCCCTGGA′] |
| 684 | NM_000274.3(OAT): c.1205T > C | 4942 | OAT | [′TGTCTACGACTTCGAGATAATGGACYTCTGG |
| | (p.Leu402Pro) | | | CCAAGCCAACCCATGGCGAC′] |
| 685 | NM_000274.3(OAT): c.1180T > C | 4942 | OAT | [′TTTAGATTGGGATGCTTGGAAGGTGYGTCTA |
| | (p.Cys394Arg) | | | CGACTTCGAGATAATGGACT′] |
| 686 | NM_001385.2(DPYS): c.1078T > | 1807 | DPYS | [′TGTTGAAGATCGGATGTCCGTAATAYGGGA |
| | C (p.Trp360Arg) | | | AAAAGGCGTGGTGGGTTTCAC′] |
| 687 | NM_000027.3(AGA): c.916T > C | 175 | AGA | [′TCCAGAATTCTTTGGGGCTGTTATAYGTGCC |
| | (p.Cys306Arg) | | | AATGTGACTGGAAGTTACGG′] |
| 688 | NM_000027.3(AGA): c.214T > C | 175 | AGA | [′GTGTGAGAGAGAGCAGTGTGACGGCYCTGT |
| | (p.Ser72Pro) | | | AGGCTTTGGAGGAAGTCCTGA′] |
| 689 | NM_000398.6(CYB5R3): | 1727 | CYB5R3 | [′CAAGTTTCCCGCTGGAGGGAAGATGYCTCA |
| | c.382T > C (p.Ser128Pro) | | | GTACCTGGAGAGCATGCAGAT′] |
| 690 | NM_000398.6(CYB5R3): | 1727 | CYB5R3 | [′GAGTTCCGGGGCCCCAGTGGGCTGCYGGTC |
| | c.446T > C (p.Leu149Pro) | | | TACCAGGGCAAAGGTGATTCG′] |
| 691 | NM_000398.6(CYB5R3): | 1727 | CYB5R3 | [′GAAGGACCCTGATGACCACACTGTGYGCCA |
| | c.610T > C (p.Cys204Arg) | | | CCTGCTCTTTGCCAACCAGGT′] |
| 692 | NM_000398.6(CYB5R3): | 1727 | CYB5R3 | [′CCGTCACCCCAGCACATCCTGGGCCYCCCTG |
| | c.218T > C (p.Leu73Pro) | | | TCGGTGAGTCACGCCCCTGC′] |
| 693 | NM_000552.3(VWF): c.4883T > C | 7450 | VWF | [′CCTGGAGACATCCAGGTGGTGCCCAYTGGA |
| | (p.Ile1628Thr) | | | GTGGGCCCTAATGCCAACGTG′] |
| 694 | NM_000552.3(VWF): c.4837T > C | 7450 | VWF | [′CTACATGGTCACCGGAAATCCTGCCYCTGAT |
| | (p.Ser1613Pro) | | | GAGATCAAGAGGCTGCCTGG′] |
| 695 | NM_000552.3(VWF): c.3814T > C | 7450 | VWF | [′GGAACCGCCGTTGCACGATTTCTACBGCAGC |
| | (p.Cys1272Arg) | | | AGGCTACTGGACCTGGTCTT′] |
| 696 | NM_000552.3(VWF): c.8317T > C | 7450 | VWF | [′CAACGATGTGCAGGACCAGTGCTCCYGCTG |
| | (p.Cys2773Arg) | | | CTCTCCGACACGGACGGAGCC′] |
| 697 | NM_000552.3(VWF): c.3445T > C | 7450 | VWF | [′TGAGTGTGAGTGGCGCTATAACAGCYGTGC |
| | (p.Cys1149Arg) | | | ACCTGCCTGTCAAGTCACGTG′] |
| 698 | NM_000552.3(VWF): c.3178T > C | 7450 | VWF | [′GAAGCAGACGATGGTGGATTCCTCCYGTAG |
| | (p.Cys1060Arg) | | | AATCCTTACCAGTGACGTCTT′] |
| 699 | NM_001042492.2(NF1): c.5858T | 4763 | NF1 | [′TGTTTGGAATACATGACTCCATGGCYGTCAA |
| | > C (p.Leu1953Pro) | | | ATCTAGTTCGTTTTTGCAAG′] |
| 700 | NM_000267.3(NF1): c.1523T > C | 4763 | NF1 | [′ATTCATGCAGATCCAAAGCTCTTGCYTTGTG |
| | (p.Leu508Pro) | | | TAAGTATTTTTTTATGAAAT′] |
| 701 | NM_000267.3(NF1): c.6200T > C | 4763 | NF1 | [′ATTTTAGCACGCTACATGCTGATGCYGTCCT |
| | (p.Leu2067Pro) | | | TCAACAATTCCCTTGATGTG′] |
| 702 | NM_000267.3(NF1): c.1070T > C | 4763 | NF1 | [′CTGTTGGGGTTTTTATAGAACCTGCYTTTTA |
| | (p.Leu357Pro) | | | ATCCAAGTAAGCCATTCTCA′] |
| 703 | NM_000267.3(NF1): c.3728T > C | 4763 | NF1 | [′TCTCAGGATGAACTAGCTCGAGTTCYGGTTA |
| | (p.Leu1243Pro) | | | CTCTGTTTGATTCTCGGCAT′] |
| 704 | NM_000308.2(CTSA): c.247T > C | 5476 | CTSA | [′CTCCGGCTCCAAGCACCTCCACTACYGGTCT |
| | (p.Trp83Arg) | | | GCCGCCCTGCCTTCTGGGCG′] |
| 705 | NM_001127695.1(CTSA): | 5476 | CTSA | [′ATCTCCTACAGGCTTTGGTCTTCTCYCCAGA |
| | Tc.707 > C (p.Leu236Pro) | | | CCCACTGCTGCTCTCAAAAC′] |
| 706 | NM_000308.2(CTSA): c.1271T > | 5476 | CTSA | [′GATGTAGACATGGCCTGCAATTTCAYGGGG |
| | C (p.Met424Thr) | | | GATGAGTGGTTTGTGGATTCC′] |
| 707 | NM_000405.4(GM2A): c.412T > | 2760 | GM2A | [′GCGTACCTATGGGCTTCCTTGCCACYGTCCC |
| | C (p.Cys138Arg) | | | TTCAAAGAAGTAAGTACTTA′] |
| 708 | NM_001165974.1(UROC1): | 131669 | UROC1 | [′CCAGAGTTTGCCCAGGAGCTGCAACYGTAC |
| | c.209T > C (p.Leu70Pro) | | | GGACACATCTACATGTACCGG′] |
| 709 | NM_003730.4(RNASET2): | 8635 | RNASET2 | [′ATATGGAGTGATACCCAAAATCCAGYGCCT |
| | c.550T > C (p.Cys184Arg) | | | TCCACCAAGCCAGGTTAGACA′] |
| 710 | NM_199069.1(NDUFAF3): | 25915 | NDUFAF3 | [′GACTTCGCCGCGCGTTGGTCAGCCAYGGCC |
| | c.2T > C (p.Met1Thr) | | | ACCGCTCTCGCGCTACGTAGC′] |
| 711 | NM_178012.4(TUBB2B): | 347733 | TUBB2B | [′CATGAACACCTTCAGCGTCATGCCCYCACCC |
| | c.514T> C (p.Ser172Pro) | | | AAGGTGTCAGACACGGTGGT′] |
| 712 | NM_178012.4(TUBB2B): | 347733 | TUBB2B | [′CCCACCTACGGGGACCTCAACCACCYGGTG |
| | c.683T > C (p.Leu228Pro) | | | TCGGCCACCATGAGCGGGGTC′] |
| 713 | NM_178012.4(TUBB2B): | 347733 | TUBB2B | [′CATGGTGCCCTTCCCTCGCCTGCACYTCTTC |
| | c.793T > C (p.Phe265Leu) | | | ATGCCCGGCTTCGCGCCCCT′] |
| 714 | NM_000110.3(DPYD): c.85T > C | 1806 | DPYD | [′TCGAACACAAACTCATGCAACTCTGYGTTCC |
| | (p.Cys29Arg) | | | ACTTCGGCCAAGAAATTAGA′] |
| 715 | NM_133459.3(CCBE1): c.520T > | 147372 | CCBE1 | [′CATCCGGGAAGATGATGGGAAGACAYGTAC |
| | C (p.Cys174Arg) | | | CAGGGGAGACAAATATCCCAA′] |
| 716 | NM_000035.3(ALDOB): c.442T > | 229 | ALDOB | [′GAAAGATGGTGTTGACTTTGGGAAGYGGCG |
| | C (p.Trp148Arg) | | | TGCTGTGCTGAGGATTGCCGA′] |
| 717 | NM_000320.2(QDPR): c.106T > C | 5860 | QDPR | [′AACCAAAGCTGTTTTCTCCTTCCAGYGGGTT |
| | (p.Trp36Arg) | | | GCCAGCGTTGATGTGGTGGA′] |
| 718 | NM_173560.3(RFX6): | 222546 | RFX6 | [′GCAGACACAGCTCACGCTGCAGTGGYGAGA |
| | c.380+2T > C | | | CTCGCCCGCAGGGTACACTGA′] |
| 719 | NM_173560.3(RFX6): c.649T > C | 222546 | RFX6 | [′AAAATGTACTAATTTTTTAAGGTTTYCTGGA |
| | (p.Ser217Pro) | | | AGCAAGCTAAAGAATGAGGT′] |
| 720 | NM_000037.3(ANK1): | 286 | ANK1 | [′CTGCGGGGCCTGTGACGTGCGGGCCRGGCC |
| | c.−108T > C | | | CCCGAGGGCCTTATCGGCCCC′] |
| 721 | NM_001145308.4(LRTOMT): | 220074 | LRTOMT | [′TCACATCCTCACCACCCTGGACCACYGGAGC |
| | c.313T > C (p.Trp105Arg) | | | AGCCGCTGCGAGTACTTGAG′] |
| 722 | NM_001012515.2(FECH): | 2235 | FECH | [′CCTGTCTGCAGGGAGACTAAATCCTYCTTCA |
| | c.1268T > C (p.Phe423Ser) | | | CCAGCCAGCAGCTGTGACCC′] |
| 723 | NM_000140.3(FECH): | 2235 | FECH | [′TATTGAGTAGAAAACATTTCTCAGGYTGCTA |
| | c.315 − 48T > C | | | AGCTGGAATAAAATCCACTT′] |
| 724 | NM_024120.4(NDUFAF5): | 79133 | NDUFAF5 | [′GCTGTCAATGACCTGGGACATCTGCYTGGG |
| | c.686T > C (p.Leu229Pro) | | | AGAGCTGGCTTTAATACTCTG′] |
| 725 | NM_000277.1(PAH): c.932T > C | 5053 | PAH | [′TTACAGGAAATTGGCCTTGCCTCTCYGGGTG |
| | (p.Leu311Pro) | | | CACCTGATGAATACATTGAA′] |
| 726 | NM_000277.1(PAH): c.764T > C | 5053 | PAH | [′GGCCTGCTTTCCTCTCGGGATTTCTYGGGTG |
| | (p.Leu255Ser) | | | GCCTGGCCTTCCGAGTCTTC′] |
| 727 | NM_000277.1(PAH): c.143T > C | 5053 | PAH | [′TCACTCAAAGAAGAAGTTGGTGCATYGGCC |
| | (p.Leu48Ser) | | | AAAGTATTGCGCTTATTTGAG′] |
| 728 | NM_000277.1(PAH): c. 1045T > C | 5053 | PAH | [′AAAGGCATATGGTGCTGGGCTCCTGBCATCC |
| | (p.Ser349Pro) | | | TTTGGTGAATTACAGGTATG′] |
| 729 | NM_000277.1(PAH): c.293T > C | 5053 | PAH | [′GCTCTGACAAACATCATCAAGATCTYGAGG |
| | (p.Leu98Ser) | | | CATGACATTGGTGCCACTGTC′] |
| 730 | NM_000277.1(PAH): c.734T > C | 5053 | PAH | [′TGCACTGGTTTCCGCCTCCGACCTGHGGCTG |
| | (p.Val245Ala) | | | GCCTGCTTTCCTCTCGGGAT′] |
| 731 | NM_000277.1(PAH): c.194T > C | 5053 | PAH | [′GAGAATGATGTAAACCTGACCCACANTGAA |
| | (p.Ile65Thr) | | | TCTAGACCTTCTCGTTTAAAG′] |
| 732 | NM_000130.4(F5): c.1160T > C | 2153 | F5 | [′CATTTGGATAATTTCTCAAACCAAAYTGGAA |
| | (p.Ile387Thr) | | | AACATTATAAGAAAGTTATG′] |
| 733 | NM_000512.4(GALNS): c.413T > | 2588 | GALNS | [′AAGGCCGGCTACGTCAGCAAGATTGYCGGC |
| | C (p.Val138Ala) | | | AAGTGGTAAGTCTCCTGGCCA′] |
| 734 | NM_018077.2(RBM28): | 55131 | RBM28 | [′TCCTTTGACTCAGAAGAAGAAGAACYTGGG |
| | c.1052T > C (p.Leu351Pro) | | | GAGCTTCTCCAACAGTTTGGA′] |
| 735 | NM_203395.2(IYD): c.347T > C | 389434 | IYD | [′AATGAGCAAGTCCCAATGGAAGTCAYTGAT |
| | (p.Ile116Thr) | | | AATGTCATCAGAACGGCAGGT′] |
| 736 | NM_015702.2(MMADHC): | 27249 | MMADHC | [′CATTTAGGATTCTCTGTTGATGACCYTGGAT |
| | c.776T > C (p.Leu259Pro) | | | GCTGTAAAGTGATTCGTCAT′] |
| 737 | NM_014165.3(NDUFAF4): | 29078 | NDUFAF4 | [′AAAGATGAAAAGCTGCTGTCGTTTCYAAAA |
| | c.194T > C (p.Leu65Pro) | | | GATGTGTATGTTGATTCCAAA′] |
| 738 | NM_001136271.2(NKX2-6): | 137814 | NKX2-6 | [′GCAGGTGCTGGCCCTGGAGCGGCGCYTCAA |
| | c.451T > C (p.Phe151Leu) | | | GCAGCAGCGGTACCTGTCAGC′] |
| 739 | NM_013319.2(UBIAD1): | 29914 | UBIAD1 | [′TATCTACTTTGGAGGCCTGTCTGGCYCCTTT |
| | c.511T > C (p.Ser171Pro) | | | CTCTACACAGGAGGTAAGAT′] |
| 740 | NM_001127628.1(FBP1): | 2203 | FBP1 | [′TTGTCTAAAAAGGCCATCGGGGAGTYCATTT |
| | c.581T > C (p.Phe194Ser) | | | TGGTGGACAAGGATGTGAAG′] |
| 741 | NM_000046.3(ARSB): c.349T > C | 411 | ARSB | [′TTTACAGCACCAAATAATCTGGCCCYGTCAG |
| | (p.Cys117Arg) | | | CCCAGCTGTGTTCCTCTGGA′] |
| 742 | NM_000046.3(ARSB): c.707T > C | 411 | ARSB | [′TCTTTCCAGCCTCTGTTTCTCTACCYTGCTCT |
| | (p.Leu236Pro) | | | CCAGTCTGTGCATGAGCCC′] |
| 743 | NM_000404.2(GLB1): c.152T > C | 2720 | GLB1 | [′AAGGATGGCCAGCCATTTCGCTACAYCTCA |
| | (p.Ile51Thr) | | | GGAAGCATTCACTACTCCCGT′] |
| 744 | NM_000404.2(GLB1): c.247T > C | 2720 | GLB1 | [′AAGGGGCTGTGTGTGTCTTGGCAGGBATGTG |
| | (p.Tyr83His) | | | CCCTGGAACTTTCATGAGCC′] |
| 745 | NM_024782.2(NHEJ1): c.367T > | 79840 | NHEJ1 | [′CCTCCCCTTCTATTGGAATTTCCACYGCATG |
| | C (p.Cys123Arg) | | | CTAGCTAGTCCTTCCCTGGT′] |
| 746 | NM_139241.3(FGD4): c.893T > C | 121512 | FGD4 | [′CAGAAATTGGCACCATTCCTTAAGABGTATG |
| | (p.Met298Thr) | | | GAGAATATGTGAAAGGATTT′] |
| 747 | NM_020631.4(PLEKHG5): | 57449 | PLEKHG5 | [′TTGGGCCTTCCCTAACCAGGGTCCTYCCTCC |
| | c.1940T > C (p.Phe647Ser) | | | TTATCTACCTGAATGAGTTT′] |
| 748 | NM_015559.2(SETBP1): | 26040 | SETBP1 | [′GAGACGATCCCCAGCGACAGCGGCAYTGGG |
| | c.2612T > C (p.Ile871Thr) | | | ACAGACAACAACAGCACTTCT′] |
| 749 | NM_138387.3(G6PC3): c.554T > | 92579 | G6PC3 | [′CTCCTAGGCGCTGTCCTGGGCTGGCYGATGA |
| | C (p.Leu185Pro) | | | CTCCCCGAGTGCCTATGGAG′] |
| 750 | NM_022489.3(INF2): c.556T > C | 64423 | INF2 | [′CTTCAGCATTGTCATGAACGAGCTCYCCGGC |
| | (p.Ser186Pro) | | | AGCGACAACGTGCCCTACGT′] |
| 751 | NM_022489.3(INF2): c.125T > C | 64423 | INF2 | [′GACCCCGAGCTGTGCATCCGGCTGCYCCAG |
| | (p.Leu42Pro) | | | ATGCCCTCTGTGGTCAACTAC′] |
| 752 | NM_018122.4(DARS2): | 55157 | DARS2 | [′TTTGAAATTAAGAACTTCGTGAAGGBACCA |
| | c.492+2T > C | | | ACCTCTGTTATTAATAAAATA′] |
| 753 | NM_015272.3(RPGRIP1L): | 23322 | RPGRIP1L | [′CCTTCATCCCGAATATAACTTCACTYCTCAA |
| | c.1975T > C (p.Ser659Pro) | | | TATCTTGTTCATGTTAATGA′] |
| 754 | NM_001029871.3(RSPO4): | 343637 | RSPO4 | [′CTTCAGCCAGGACTTCTGCATCCGGYGCAAG |
| | c.319T > C (p.Cys107Arg) | | | AGGCAGTTTTACTTGTACAA′] |
| 755 | NM_000429.2(MAT1A): c.914T > | 4143 | MATIA | [′GCTGCCCGCTGGGTGGCCAAGTCTCYGGTG |
| | C (p.Leu305Pro) | | | AAAGCAGGGCTCTGCCGGAGA′] |
| 756 | NM_032581.3(FAM126A): | 84668 | FAM126A | [′AACATTTTGTCTTTCTTCTAGTTGCBAGAAC |
| | c.158T > C (p.Leu53Pro) | | | CTGTCTGTCACCAGCTCTTT′] |
| 757 | NM_006364.2(SEC23A): | 10484 | SEC23A | [′GGGTGATTCTTTCAATACTTCCTTAYTCAAA |
| | c.1144T > C (p.Phe382Leu) | | | CAAACTTTTCAAAGAGTCTT′] |
| 758 | NM_018006.4(TRMU): c.229T > | 55687 | TRMU | [′CCATCAAGTGTCCTACGTAAAGGAGYATTG |
| | C (p.Tyr77His) | | | GAATGATGTGTTCAGGTGAGT′] |
| 759 | NM_001174089.1(SLC4A11): | 83959 | SLC4A11 | [′CTGCAGGTGCTTCAGCTGCTGCTGCYGTGTG |
| | c.2480T > C (p.Leu827Pro) | | | CCTTCGGCATGAGCTCCCTG′] |
| 760 | NM_001174089.1(SLC4A11): | 83959 | SLC4A11 | [′AGTGACAGGGGTGCGGTACCAGCAGYCGTG |
| | c.589T > C (p.Ser197Pro) | | | GCTCTGCATCATGTGAGTTGC′] |
| 761 | NM_148960.2(CLDN19): | 149461 | CLDN19 | [′GCCCTGATGGTGGTGGCCGTGCTCCYGGGCT |
| | c.269T > C (p.Leu90Pro) | | | TCGTGGCCATGGTCCTCAGC′] |
| 762 | NM_153704.5(TMEM67): | 91147 | TMEM67 | [′AACACAGATGGTCAGACTTTTGAGAYTGCA |
| | c.2498T > C (p.Ile833Thr) | | | ATTTCTAACCAGATGAGACAA′] |
| 763 | NM_153704.5(TMEM67): | 91147 | TMEM67 | [′ACAGGTCTTTACTGGCTTATTTTCTYCAAAG |
| | c.1769T > C (p.Phe590Ser) | | | TGAGTGAGTTTCTGAATTTT′] |
| 764 | NM_153704.5(TMEM67): | 91147 | TMEM67 | [′AGAACGTTTTGTCACTTATGTTGGAHGTGCC |
| | c.1843T > C (p.Cys615Arg) | | | TTTGCTCTGAAGGTAAGTTT′] |
| 765 | NM_153704.5(TMEM67): | 91147 | TMEM67 | [′ACATCTTGTCAAGCTCTTGGAAATAYGTGTG |
| | c.755T > C (p.Met252Thr) | | | TGATGAACATGAATTCTTAC′] |
| 766 | NM_017777.3(MKS1): | 54903 | MKS1 | [′CCCCGTGCGCAACTTGCGCCTCCGGYAGTCG |
| | c.80+2T > C | | | CACCGCCCCAGCCCCGAGGC′] |
| 767 | NM_001041.3(SI): c.1022T > C | 6476 | SI | [′AATTACATGCTCATTTACTTTAAGCYTGTTG |
| | (p.Leu341Pro) | | | GACTACCAGCAATGCCAGCA′] |
| 768 | NM_001041.3(SI): c.1859T > C | 6476 | SI | [′ATGGAATGGTCTATAACTGGAATGCYGGAG |
| | (p.Leu620Pro) | | | TTCAGTTTGTTTGGAATACCT′] |
| 769 | NM_015506.2(MMACHC): | 25974 | MMACHC | [′CACCCCAACCGACGCCCCAAGATCCYGGCC |
| | c.347T > C (p.Leul16Pro) | | | CAGACAGCAGCCCATGTAGCT′] |
| 770 | NM_000190.3(HMBS): c.739T > | 3145 | HMBS | [′GCACGATCCCGAGACTCTGCTTCGCYGCATC |
| | C (p.Cys247Arg) | | | GCTGAAAGGGCCTTCCTGAG′] |
| 771 | NM_000190.3(HMBS): c.242T > | 3145 | HMBS | [′GAGAAAAGCCTGTTTACCAAGGAGCYTGAA |
| | C (p.Leu81Pro) | | | CATGCCCTGGAGAAGAATGAG′] |
| 772 | NM_000237.2(LPL): c.662T > C | 4023 | LPL | [′ACCAGAGGGTCCCCTGGTCGAAGCAYTGGA |
| | (p.Ile221Thr) | | | ATCCAGAAACCAGTTGGGCAT′] |
| 773 | NM_000237.2(LPL): c.337T > C | 4023 | LPL | [′AGACTCCAATGTCATTGTGGTGGACYGGCTG |
| | (p.Trp113Arg) | | | TCACGGGCTCAGGAGCATTA′] |
| 774 | NM_000237.2(LPL): c.755T > C | 4023 | LPL | [′AACATTGGAGAAGCTATCCGCGTGAYTGCA |
| | (p.Ile252Thr) | | | GAGAGAGGACTTGGAGGTAAA′] |
| 775 | NM_000263.3(NAGLU): | 4669 | NAGLU | [′GCTGGGGCCAGGCCCCGCGGCCGACYTCTC |
| | c.142T > C (p.Phe48Leu) | | | CGTGTCGGTGGAGCGCGCTCT′] |
| 776 | NM_000018.3(ACADVL): | 37 | ACADVL | [′GCGTGTGCTCCGAGATCTTCGCATCYTCCGG |
| | c.1372T > C (p.Phe458Leu) | | | ATCTTTGAGGGGACAAATGA′] |
| 777 | NM_018105.2(THAP1): c.241T > | 55145 | THAP1 | [′GAAAGAGAATGCTGTGCCCACAATAYTTCTT |
| | C (p.Phe81Leu) | | | TGTACTGAGCCACATGACAA′] |
| 778 | NM_005908.3(MANBA): | 4126 | MANBA | [′AGACAAGAGTCGTCCTTTTATTACGYCCAGT |
| | c.1513T > C (p.Ser505Pro) | | | CCTACAAATGGGGCTGAAAC′] |
| 779 | NM_000124.3(ERCC6): | 2074 | ERCC6 | [′AAGCAGTTTTTGACAAATAGAGTGCYAAAA |
| | c.2960T > C (p.Leu987Pro) | | | GACCCAAAACAAAGGCGGTTT′] |
| 780 | NM_014845.5(FIG4): c.122T > C | 9896 | FIG4 | [′GAAACGAAATATCGTGTCTTGAAGAYTGAT |
| | (p.Ile41Thr) | | | AGAACAGAACCAAAAGATTTG′] |
| 781 | NM_000787.3(DBH): | 1621 | DBH | [′GATGGGGACACTGCCTATTTTGCGGYGAGTC |
| | c.339 + 2T > C | | | TCTCCTCCCTGCCAGCTCTC′] |
| 782 | NM_000102.3(CYP17A1): | 1586 | CYP17AI | [′TCTAAAGGCAACTCTAGACATCGCGYCCAA |
| | c.316T > C (p.Ser106Pro) | | | CAACCGTAAGGGTATCGCCTT′] |
| 783 | NM_000102.3(CYP17A1): | 1586 | CYP17A1 | [′GGCGCTGCATCACAATGAGAAGGAGYGGCA |
| | c.1216T > C (p.Trp406Arg) | | | CCAGCCGGATCAGTTCATGCC′] |
| 784 | NM_000102.3(CYP17A1): | 1586 | CYP17A1 | [′GAGATCCTGGCCCGCCAGGAGCTCTYCCTCA |
| | c.1358T > C (p.Phe453Ser) | | | TCATGGCCTGGCTGCTGCAG′] |
| 785 | NM_152783.4(D2HGDH): | 728294 | D2HGDH | [′GGAGATGGTAACCTGCACCTCAATGYGACG |
| | c.1331T > C (p.Val444Ala) | | | GCGGAGGCCTTCAGCCCCTCG′] |
| 786 | NM_000255.3(MUT): c.313T > C | 4594 | MUT | [′TCCTACCATGTATACCTTTAGGCCCYGGACC |
| | (p.Trp105Arg) | | | ATCCGCCAGTATGCTGGTTT′] |
| 787 | NM_000411.6(HLCS): c.710T > C | 3141 | HLCS | [′CTGTACCAGAAGTTCATGGCCTATCYTTCTC |
| | (p.Leu237Pro) | | | AGGGAGGGAAGGTGTTGGGC′] |
| 788 | NM_022132.4(MCCC2): c.499T > | 64087 | MCCC2 | [′AATTGCCATGCAAAACAGGCTCCCCYGCAT |
| | C (p.Cys167Arg) | | | CTACTTAGGCAAGTCACCAGA′] |
| 789 | NM_020166.4(MCCC1): | 56922 | MCCC1 | [′CATTATGACCCCATGATTGCGAAGCYGGTCG |
| | c.1310T > C (p.Leu437Pro) | | | TGTGGGCAGCAGATCGCCAG′] |
| 790 | NM_198578.3(LRRK2): | 120892 | LRRK2 | [′ATTGCAAAGATTGCTGACTACGGCAYTGCTC |
| | c.6059T > C (p.Ile2020Thr) | | | AGTACTGCTGTAGAATGGGG′] |
| 791 | NM_000022.2(ADA): c.320T > C | 100 | ADA | [′GAGGTGCGGTACAGTCCGCACCTGCYGGCC |
| | (p.Leu107Pro) | | | AACTCCAAAGTGGAGCCAATC′] |
| 792 | NM_199242.2(UNC13D): | 201294 | UNC13D | [′CTGGCCGCCTCATTCAGCTCCCTGCYGACCT |
| | c.1208T > C (p.Leu403Pro) | | | ACGGCCTCTCCCTCATCCGG′] |
| 793 | NM_152443.2(RDH12): c.523T > | 145226 | RDH12 | [′CCCTGCACGGGTGGTTAATGTGTCCYCGGTG |
| | C (p.Ser175Pro) | | | GCTCACCACATTGGCAAGAT′] |
| 794 | NM_020435.3(GJC2): c.857T > C | 57165 | GJC2 | [′TGCCTGCTGCTCAACCTCTGTGAGAYGGCCC |
| | (p.Met286Thr) | | | ACCTGGGCTTGGGCAGCGCG′] |
| 795 | NM_000159.3(GCDH): c.883T > | 2639 | GCDH | [′CTTCGGCTGCCTGAACAACGCCCGGYACGG |
| | C (p.Tyr295His) | | | CATCGCGTGGGGCGTGCTTGG′] |
| 796 | NM_000920.3(PC): c.434T > C | 5091 | PC | [′CGGTTTATTGGGCCAAGCCCAGAAGBGGTC |
| | (p.Val145Ala) | | | CGCAAGATGGGAGACAAGGTG′] |
| 797 | NM_207118.2(GTF2H5): c.62T > | 404672 | GTF2H5 | [′GATCCTGCCATGAAGCAGTTTCTGCYGTACT |
| | C (p.Leu21Pro) | | | TGGATGAGTCCAATGCCCTG′] |
| 798 | NM_005787.5(ALG3): c.211T > C | 10195 | ALG3 | [′TCTTCTTCCAGACACAGAGATTGACYGGAA |
| | (p.Trp71Arg) | | | GGCCTACATGGCCGAGGTAGA′] |
| 799 | NM_024514.4(CYP2R1): | 120227 | CYP2R1 | [′GGCTATGATGTAGTAAAGGAATGCCYTGTTC |
| | c.296T > C (p.Leu99Pro) | | | ATCAAAGCGAAATTTTTGCA′] |
| 800 | NM_017929.5(PEX26): c.2T > C | 55670 | PEX26 | [′CTGGGCCTTGGACCCGGACTCGTTAYGAAG |
| | (p.Met1Thr) | | | AGCGATTCTTCGACCTCTGCA′] |
| 801 | NM_017929.5(PEX26): c.134T > | 55670 | PEX26 | [′CTTCTGGAGGAGGCGGCCGACCTCCYGGTG |
| | C (p.Leu45Pro) | | | GTGCACCTGGACTTCCGGGCG′] |
| 802 | NM_213599.2(ANO5): c.1066T > | 203859 | ANO5 | [′TGGTCAGATGATCATGTGCCCACTCBGTGAT |
| | C (p.Cys356Arg) | | | CAAGTGTGTGATTATTGGAG′] |
| 803 | NM_022370.3(ROBO3): | 64221 | ROBO3 | [′CCAGCTGGTGCAAGGTTTCCGGGTGHCTTGG |
| | c.2113T > C (p.Ser705Pro) | | | AGGGTAGCAGGCCCTGAGGG′] |
| 804 | NM_022370.3(ROBO3): c.14T > C | 64221 | ROBO3 | [′CTGGGTCGAGCCATGCTGCGCTACCYGCTGA |
| | (p.Leu5Pro) | | | AAACGCTGCTGCAGATGAAC′] |
| 805 | NM_207352.3(CYP4V2): | 285440 | CYP4V2 | [′GTATGTTTTTCTCTTCCTAAGGTAABTTTAAC |
| | c.332T > C (p.Ile111Thr) | | | TAGTTCAAAGCAAATTGAC′] |
| 806 | NM_178857.5(RP1L1): c.2878T > | 94137 | RP1L1 | [′GTCTCCAGAGGCTGTGGTCCGCGAAYGGCT |
| | C (p.Trp960Arg) | | | GGACAACATTCCAGAAGAGCC′] |
| 807 | NM_018668.4(VPS33B): c.89T > | 26276 | VPS33B | [′GCTCGAGACCAGCTCATCTATCTGCYGGAGC |
| | C (p.Leu30Pro) | | | AGGTCAGTGCTTGCCTGACG′] |
| 808 | NM_024006.5(VKORC1): | 79001 | VKORC1 | [′CGGGATTACCGCGCGCTCTGCGACGYGGGC |
| | c.134T > C (p. Val45Ala) | | | ACCGCCATCAGCTGTTCGCGC′] |
| 809 | NM_000551.3(VHL): c.334T > C | 7428 | VHL | [′TGGCACGGGCCGCCGCATCCACAGCHACCG |
| | (p.Tyr112His) | | | AGGTACGGGCCCGGCGCTTAG′] |
| 810 | NM_000551.3(VHL): c.292T > C | 7428 | VHL | [′CAACTTCGACGGCGAGCCGCAGCCCYACCC |
| | (p.Tyr98His) | | | AACGCTGCCGCCTGGCACGGG′] |
| 811 | NM_000551.3(VHL): c.188T > C | 7428 | VHL | [′GAGGCCGGGCGGCCGCGGCCCGTGCYGCGC |
| | (p.Leu63Pro) | | | TCGGTGAACTCGCGCGAGCCC′] |
| 812 | NM_000551.3(VHL): c.488T > C | 7428 | VHL | [′GTGTATACTCTGAAAGAGCGATGCCYCCAG |
| | (p.Leu163Pro) | | | GTTGTCCGGAGCCTAGTCAAG′] |
| 813 | NM_014874.3(MFN2): c.227T > C | 9927 | MFN2 | [′CCCGTTACCACAGAAGAACAGGTTCBGGAC |
| | (p.Leu76Pro) | | | GTCAAAGGTTACCTATCCAAA′] |
| 814 | NM_015046.5(SETX): c.1166T > | 23064 | SETX | [′AACATGTATGAAGAAATGGAAACATYAGCC |
| | C (p.Leu389Ser) | | | AGTGTACTTCAGTCAGATATT′] |
| 815 | NM_005609.2(PYGM): c.1187T > | 5837 | PYGM | [′TGGCCGGTGCACCTCTTGGAGACGCYGCTGC |
| | C (p.Leu396Pro) | | | CGCGGCACCTCCAGATCATC′] |
| 816 | NM_005609.2(PYGM): c.2392T > | 5837 | PYGM | [′CTTTGACCTGCAGAACCCAAGAGAGHGGAC |
| | C (p.Trp798Arg) | | | GCGGATGGTGATCCGGAACAT′] |
| 817 | NM_213653.3(HFE2): c.842T > C | 148738 | HFE2 | [′CATGTGGAGATCCAAGCTGCCTACAYTGGC |
| | (p.Ile281Thr) | | | ACAACTATAATCATTCGGCAG′] |
| 818 | NM_213653.3(HFE2): c.238T > C | 148738 | HFE2 | [′TGGAGGGGTGGGCTCTGGCGGCCTCBGTCG |
| | (p.Cys80Arg) | | | AGCCCTCCGCTCCTATGCGCT′] |
| 819 | NM_213653.3(HFE2): c.302T > C | 148738 | HFE2 | [′ACCGCCCGCACCTGCCGCGGGGACCYCGCC |
| | (p.Leu101Pro) | | | TTCCATTCGGCGGTACATGGC′] |
| 820 | NM_000045.3(ARG1): c.32T > C | 383 | ARG1 | [′GCCAAGTCCAGAACCATAGGGATTAYTGGA |
| | (p.Ile11Thr) | | | GCTCCTTTCTCAAAGGGACAG′] |
| 821 | NM_032409.2(PINK1): c.1040T > | −1 | — | [′GCCGCCATGATGCTGCTGCAGCTGCYGGAA |
| | C (p.Leu347Pro) | | | GGCGTGGACCATCTGGTTCAA′] |
| 822 | NM_153006.2(NAGS): c.1289T > | −1 | — | [′GCCAGGTACAACGCCGCCGCCATTCYGACC |
| | C (p.Leu430Pro) | | | ATGGAGCCCGTCCTGGGGGGC′] |
| 823 | NM_153006.2(NAGS): c.1450T > | −1 | — | [′CCGGGTCACCAACCCCATCAATCCCBGGTAG |
| | C (p.Trp484Arg) | | | GTCCTGCCACTCCCAGCTCT′] |
| 824 | NM_001173464.1(KIF21A): | 55605 | KIF21A | [′AGTATTTCTGATTGTCAGGCCAACAYAATGC |
| | c.3029T > C (p.Ile1010Thr) | | | AGATGGAAGAAGCAAAGGTT′] |
| 825 | NM_001173464.1(KIF21A): | 55605 | KIF21A | [′GTCAGCCCTTCAGACAGAGACTTTAYGGAA |
| | c.1067T > C (p.Met356Thr) | | | ACGTTAAACACCCTGAAATAC′] |
| 826 | NM_000434.3(NEU1): c.1088T > | 4758 | NEU1 | [′TCATGGCGGAAAGAGACAGTCCAGCYATGG |
| | C (p.Leu363Pro) | | | CCAGGCCCCAGTGGCTATTCA′] |
| 827 | NM_000434.3(NEU1): c.718T > C | 4758 | NEU1 | [′CCTCAGCGATGATCATGGTGCCTCCYGGCGC |
| | (p.Trp240Arg) | | | TACGGAAGTGGGGTCAGCGG′] |
| 828 | NM_000026.2(ADSL): c.1312T > | 158 | ADSL | [′TGATGCCTACTTCAGTCCCATTCACYCCCAG |
| | C (p.Ser438Pro) | | | TTGGATCATTTACTGGATCC′] |
| 829 | NM_000026.2(ADSL): c.674T > C | 158 | ADSL | [′TCCAAGGTAGAGCAGCTTGACAAGAYGGTG |
| | (p.Met225Thr) | | | ACAGAAAAGGCAGGATTTAAG′] |
| 830 | NM_018400.3(SCN3B): c.29T > C | 55800 | SCN3B | [′CCTGCCTTCAATAGATTGTTTCCCCYGGCTT |
| | (p.Leu10Pro) | | | CTCTCGTGCTTATCTACTGG′] |
| 831 | NM_024577.3(SH3TC2): | 79628 | SH3TC2 | [′CCTTCCTGTATCAGGAGTCCCAGGTRTATTG |
| | c.505T > C (p.Tyr169His) | | | TTTCCAGGTGTTTATCATCT′] |
| 832 | NM_000243.2(MEFV): c.2177T > | 4210 | MEFV | [′GTGGGCATCTTCGTGGACTACAGAGHTGGA |
| | C (p.Val726Ala) | | | AGCATCTCCTTTTACAATGTG′] |
| 833 | NM_000483.4(APOC2): c.142T > | −1 | — | [′GGTGAAGGAATCTCTCTCCAGTTACYGGGA |
| | C (p.Trp48Arg) | | | GTCAGCAAAGACAGCCGCCCA′] |
| 834 | NM_058172.5(ANTXR2): | 118429 | ANTXR2 | [′CTCTTCTTTCTAAAGCTTGAAAGAAYTGCTG |
| | c.566T > C (p.Ile189Thr) | | | ATTCCAAGGAGCAAGTTTTC′] |
| 835 | NM_001128085.1(ASPA): | −1 | — | [′TCAGACTTCTCTGGCTCCACTACCCYGCTAC |
| | c.454T > C (p.Cys152Arg) | | | GTTTATCTGATTGAGCATCC′] |
| 836 | NM_022464.4(SIL1): | 64374 | SIL1 | [′TGACTTATGGACGAAGAAATACAGTRCCTG |
| | c.645 + 2T > C | | | ATGGACATAATATTCAAGATC′] |
| 837 | NM_001037633.1(SIL1): | 64374 | SIL1 | [′CTGCTGGGCTCTGTCAACAGCTTGCYGAAGG |
| | c.1370T > C (p.Leu457Pro) | | | AGCTGAGATGAGGCCCCACA′] |
| 838 | NM_000391.3(TPP1): c.1093T > C | 1200 | TPP1 | [′TCTCTCAGGTGACAGTGGGGCCGGGYGTTG |
| | (p.Cys365Arg) | | | GTCTGTCTCTGGAAGACACCA′] |
| 839 | NM_182760.3(SUMF1): | 285362 | SUMF1 | [′AGTGAAGAAAGGTGGATCCTACATGYGCCA |
| | c.1006T > C (p.Cys336Arg) | | | TAGGGTAAGTCATGTCACTAA′] |
| 840 | NM_182760.3(SUMF1): c.463T > | 285362 | SUMF1 | [′CTTATAGGCTGAGAAGTTTGGCGACYCCTTT |
| | C (p.Ser155Pro) | | | GTCTTTGAAGGCATGTTGAG′] |
| 841 | NM_177986.3(DSG4): c.574T > C | −1 | — | [′TGCAGATGAAGAAAATCATCTGAATHCTAA |
| | (p.Ser192Pro) | | | AATTGCCTACAAGATCGTCTC′] |
| 842 | NM_004183.3(BEST1): c.253T > | 7439 | BEST1 | [′CCCCCGCCCCTCCTGCCCAGGCTTCYACGTG |
| | C (p.Tyr85His) | | | ACGCTGGTCGTGACCCGCTG′] |
| 843 | NM_004183.3(BEST1): c.122T > | 7439 | BEST1 | [′TATGGCGAGTTCTTAATCTTCCTGCYCTGCT |
| | C (p.Leu41Pro) | | | ACTACATCATCCGCTTTATT′] |
| 844 | NM_004183.3(BEST1): c.614T > | 7439 | BEST1 | [′CTTGGAGGTCGAATCCGGGACCCTAYCCTGC |
| | C (p.Ile205Thr) | | | TCCAGAGCCTGCTGAACGTG′] |
| 845 | NM_004183.3(BEST1): c.704T > | 7439 | BEST1 | [′TACGACTGGATTAGTATCCCACTGGYGTATA |
| | C (p.Val235Ala) | | | CACAGGTGAGGACTAGGCTG′] |
| 846 | NM_024312.4(GNPTAB): | 79158 | GNPTAB | [′TGTTTGTTGTTGTTAAAAGGATGTTYTTCGA |
| | T c.1120> C (p.Phe374Leu) | | | AATTTGAGCCACTTGCCTAC′] |
| 847 | NM_000158.3(GBE1): c.671T > C | 2632 | GBE1 | [′TATAAACATTTTACATGCAATGTACYACCAA |
| | (p.Leu224Pro) | | | GAATCAAAGGCCTTGGTAAG′] |
| 848 | NM_017890.4(VPS13B): | 157680 | VPS13B | [′CAGGTGCCATCTTCAAACAGTTCCAYTATTT |
| | c.8459T > C (p.Ile2820Thr) | | | ATGTCTGGTGCACAGTTTTG′] |
| 849 | NM_000019.3(ACAT1): c.935T > | 38 | ACAT1 | [′CTCAATGTTACACCACTGGCAAGAAYAGTA |
| | C (p.Ile312Thr) | | | GGTAAGGCCAGGCGAGGTGGC′] |
| 850 | NM_020184.3(CNNM4): | 26504 | CNNM4 | [′CTCAGTTTTCCCATTAGCAAGCTCCYGGACT |
| | c.971T > C (p.Leu324Pro) | | | TTTTTCTGGGCCAGGAGATT′] |
| 851 | NM_001171507.2(MCFD2): | 90411 | MCFD2 | [′GATGACAAGAACAATGATGGATACAYTGAC |
| | c.407T > C (p.Ile136Thr) | | | TATGCTGAATTTGCAAAATCA′] |
| 852 | NM_174936.3(PCSK9): c.646T > | 255738 | PCSK9 | [′TGTGCCCGAGGAGGACGGGACCCGCYTCCA |
| | C (p.Phe216Leu) | | | CAGACAGGTAAGCACGGCCGT′] |
| 853 | NM_000419.3(ITGA2B): | 3674 | ITGA2B | [′GACTTCCGGGACAAGCTGAGCCCCAYTGTG |
| | Tc.1787 > C (p.Ile596Thr) | | | CTCAGCCTCAATGTGTCCCTA′] |
| 854 | NM_000419.3(ITGA2B): | 3674 | ITGA2B | [′CCCTTCCAGGCCGGAGAGCTGGTGCYTGGG |
| | c.641T > C (p.Leu214Pro) | | | GCTCCTGGCGGCTATTATTTC′] |
| 855 | NM_004817.3(TJP2): c.143T > C | 9414 | TJP2 | [′TCCAAAAGAGGATTTGGAATTGCAGYGTCC |
| | (p.Val48Ala) | | | GGAGGCAGAGACAACCCCCAC′] |
| 856 | NM_173477.4(USH1G): c.143T > | 124590 | USH1G | [′GCCTACCATGGCAACCTCGAGTCGCYGCGTC |
| | C (p.Leu48Pro) | | | TCATTGTGAGCCGCGGGTGA′] |
| 857 | NM_000271.4(NPC1): c.3182T > | 4864 | NPC1 | [′GACGCTCTGAAGAAAGCCCGACTTAYAGCC |
| | C (p.Ile1061Thr) | | | AGTAATGTCACCGAAACCATG′] |
| 858 | NM_000271.4(NPC1): c.1133T > | 4864 | NPC1 | [′TTTGTCCGGGTCACAACCAATCCAGYTGACC |
| | C (p.Val378Ala) | | | TCTGGTCAGCCCCCAGCAGC′] |
| 859 | NM_000271.4(NPC1): c.337T > C | 4864 | NPC1 | [′ACTGAACCTGTTTTGTGAGCTGACAYGTAGC |
| | (p.Cys113Arg) | | | CCTCGACAGAGTCAGTTTTT′] |
| 860 | NM_000543.4(SMPD1): c.911T > | 6609 | SMPD1 | [′CGGGCCCTGACCACCGTCACAGCACYTGTG |
| | C (p.Leu304Pro) | | | AGGAAGTTCCTGGGGCCAGTG′] |
| 861 | NM_000226.3(KRT9): c.503T > C | 3857 | KRT9 | [′CTCAATTCTCGGCTGGCCTCTTACTYGGATA |
| | (p.Leu168Ser) | | | AGGTGCAGGCTCTAGAGGAG′] |
| 862 | NM_000226.3(KRT9): c.470T > C | 3857 | KRT9 | [′CTGACTGCTAATGAGAAGAGCACCANGCAG |
| | (p.Met157Thr) | | | GAACTCAATTCTCGGCTGGCC′] |
| 863 | NM_000051.3(ATM): c.7967T > C | −1 | — | [′CCAGCAGACCAGCCAATTACTAAACYTAAG |
| | (p.Leu2656Pro) | | | AATTTAGAAGATGTTGTTGTC′] |
| 864 | NM_000487.5(ARSA): c.410T > C | 410 | ARSA | [′GGGGTGGGGCCTGAGGGGGCCTTCCYGCCC |
| | (p.Leu137Pro) | | | CCCCATCAGGGCTTCCATCGA′] |
| 865 | NM_017653.3(DYM): c.1624T > | 54808 | DYM | [′AATGATGTTAGAGATCATCAACTCCYGCCTG |
| | C (p.Cys542Arg) | | | ACAAATTCCCTTCACCACAA′] |
| 866 | NM_016038.2(SBDS): | 51119 | SBDS | [′GACCAAACTGAAATCTGTAAGCAGGYGGGT |
| | c.258 + 2T > C | | | AACAGCTGCAGCATAGCTAAC′] |
| 867 | NM_001079802.1(FKTN): | 2218 | FKTN | [′ACTCATGCGATCCACTTGGTAGTCTYTCATG |
| | c.527T > C (p.Phe176Ser) | | | AGAGGAGTGGCAACTACCTC′] |
| 868 | NM_013382.5(POMT2): | 29954 | POMT2 | [′GGCAGGACTAAGGTGGCTGGACTCAYGGGA |
| | c.2242T > C (p.Trp748Arg) | | | CTTTTGAGGCCACTGCAAAGA′] |
| 869 | NM_024529.4(CDC73): c.191T > | 79577 | CDC73 | [′ACATTGGATTCCATTTTATTTCTACYTAATA |
| | C (p.Leu64Pro) | | | ACGTGCACCTTTCTCATCCT′] |
| 870 | NM_000268.3(NF2): c.1079T > C | 4771 | NF2 | [′ACGAGGGATGAGTTGGAGAGGAGGCYGCTG |
| | (p.Leu360Pro) | | | CAGATGAAAGAAGAAGCAACA′] |
| 871 | NM_000268.3(NF2): c.1604T > C | 4771 | NF2 | [′GAATACATGGAAAAGAGCAAGCATCYGCAG |
| | (p.Leu535Pro) | | | GAGCAGCTCAATGAACTCAAG′] |
| 872 | NM_000268.3(NF2): c.185T > C | 4771 | NF2 | [′CTGGGGCTCCGAGAAACCTGGTTCTYTGGAC |
| | (p.Phe62Ser) | | | TGCAGTACACAATCAAGGAC′] |
| 873 | NM_139248.2(LIPH): c.322T > C | 200879 | LIPH | [′AGACATGAACGTAGTTGTTGTTGATYGGAAT |
| | (p.Trp108Arg) | | | CGAGGAGCTACAACTTTAAT′] |
| 874 | NM_015102.4(NPHP4): | 261734 | NPHP4 | [′GCCACGCTGGGGGTCGCCGAGTTCTYTGAGT |
| | c.2972T > C (p.Phe991Ser) | | | TTGTGCTTAAGAACCCCCAC′] |
| 875 | NM_024105.3(ALG12): c.473T > | 79087 | ALG12 | [′ACCCTCCCGCACTGTCTTGCAGTCCYGCTGG |
| | C (p.Leu158Pro) | | | CCCTCGCGGCCTGGCTGCGG′] |
| 876 | NM_000280.4(PAX6): c.773T > C | 5080 | PAX6 | [′GTCCCACCTGATTTCCAGGTATGGTYTTCTA |
| | (p.Phe258Ser) | | | ATCGAAGGGCCAAATGGAGA′] |
| 877 | NM_024426.4(WT1): c.1351T > C | 7490 | WT1 | [′CTCTGTCCATTTAGGTGTGAAACCAYTCCAG |
| | (p.Phe451Leu) | | | TGTAAAACTTGTCAGCGAAA′] |
| 878 | NM_024426.4(WT1): c.1378T > C | 7490 | WT1 | [′CCAGTGTAAAACTTGTCAGCGAAAGYTCTCC |
| | (p.Phe460Leu) | | | CGGTCCGACCACCTGAAGAC′] |
| 879 | NM_005957.4(MTHFR): c.968T > | 4524 | MTHFR | [′GTGCCAGGCCTCCACTTCTACACCCYCAACC |
| | C (p.Leu323Pro) | | | GCGAGATGGCTACCACAGAG′] |
| 880 | NM_016247.3(IMPG2): c.370T > | 50939 | IMPG2 | [′TCACGCCCAGGAAGTCGATCCCAAARAGTC |
| | C (p.Phe124Leu) | | | CTGAAGGCTTCCCAGACAGCT′] |
| 881 | NM_002225.3(IVD): c.134T > C | 3712 | IVD | [′CCCGTGGACGATGCAATCAATGGGCYAAGC |
| | (p.Leu45Pro) | | | GAGGAGCAGAGGCAGGTGAGG′] |
| 882 | NM_001127328.2(ACADM): | 34 | ACADM | [′CAGTTAGCTACTGATGCTGTGCAGAYACTTG |
| | c.1136T > C (p.Ile379Thr) | | | GAGGCAATGGATTTAATACA′] |
| 883 | NM_001127328.2(ACADM): | 34 | ACADM | [′TTAGGAATTAAACATGGGCCAGCGAYGTTC |
| | c.742T > C (p.Cys248Arg) | | | AGATACTAGAGGAATTGTCTT′] |
| 884 | NM_000016.5(ACADM): | 34 | ACADM | [′GGAAATCATCCCAGTGGCTGCAGAAYATGA |
| | c.199T > C (p.Tyr67His) | | | TAAAACTGGTGAAGTAGGTAT′] |
| 885 | NM_000155.3(GALT): c.221T > C | 2592 | GALT | [′CCCCGCCATGACCCTCTCAACCCTCBGTGTC |
| | (p.Leu74Pro) | | | CTGGGGCCATCCGAGCCAAC′] |
| 886 | NM_000155.3(GALT): c.512T > C | 2592 | GALT | [′CTCCGTATCCCTATCTGATAGATCTYTGAAA |
| | (p.Phe171Ser) | | | ACAAAGGTGCCATGATGGGC′] |
| 887 | NM_000155.3(GALT): c.580T > C | 2592 | GALT | [′CCCTTGACAGGTATGGGCCAGCAGTYTCCTG |
| | (p.Phe194Leu) | | | CCAGATATTGCCCAGCGTGA′] |
| 888 | NM_000250.1(MPO): c.752T > C | 4353 | MPO | [′ACTCCGGACCAGGAGCGCTCACTCAYGTTC |
| | (p.Met251Thr) | | | ATGCAATGGGGCCAGCTGTTG′] |
| 889 | NM_020247.4(ADCK3): | 56997 | ADCK3 | [′CTCAGCCAGGAGATTCGGAACGAGGYTTGT |
| | c.1398 + 2T > C | | | CTGTGCCAGCAGACAGGTGGG′] |
| 890 | NM_000229.1(LCAT): c.508T > C | 3931 | LCAT | [′GACTGTGCGCGCCGCCCCCTATGACYGGCG |
| | (p.Trp170Arg) | | | GCTGGAGCCCGGTGAGTGTCT′] |
| 891 | NM_000229.1(LCAT): c.698T > C | 3931 | LCAT | [′CGCTTTATTGATGGCTTCATCTCTCYTGGGG |
| | (p.Leu233Pro) | | | CTCCCTGGGGTGGCTCCATC′] |
| 892 | NM_000229.1(LCAT): | 3931 | LCAT | [′CAGGTGCCCCAGACCCCAGCTGCCCYGACC |
| | c.524 − 22T > C | | | CCTTCCACCCGCTGCAGGCCA′] |
| 893 | NM_000403.3(GALE): c.548T > C | 2582 | GALE | [′CCACAGACTTGGAACGCAGTGCTGCYGCGC |
| | (p.Leu183Pro) | | | TATTTCAACCCCACAGGTGCC′] |
| 894 | NM_000527.4(LDLR): | 3949 | LDLR | [′ACAAATCTGACGAGGAAAACTGCGGYATGG |
| | c.694 + 2T > C | | | GCGGGGCCAGGGTGGGGGCGG′] |
| 895 | NM_000375.2(UROS): c.217T > C | 7390 | UROS | [′CAGAGCAGTGGAAGCAGCAGAGTTAYGTTT |
| | (p.Cys73Arg) | | | GGAGCAAAACAATAAAACTGA′] |
| 896 | NM_000375.2(UROS): | 7390 | UROS | [′GCTAACATGCTCTTTCTTGGCCTTAYCAGTG |
| | c.−26 − 177T > C | | | ACAGGGGTCTTCAGAAAGAA′] |
| 897 | NM_000372.4(TYR): c.265T > C | 7299 | TYR | [′GCCTTCCGTCTTTTATAATAGGACCYGCCAG |
| | (p.Cys89Arg) | | | TGCTCTGGCAACTTCATGGG′] |
| 898 | NM_000017.3(ACADS): c.529T > | 35 | ACADS | [′CACCGCCCGGGCCGAGGGCGACTCABGGGT |
| | C (p.Trp177Arg) | | | TCTGAATGGAACCAAAGCCTG′] |
| 899 | NM_000053.3(ATP7B): | 540 | ATP7B | [′GCGCCTCAGCCACTCACGGTTTCCARTCAGC |
| | c.3443T > C (p.Ile1148Thr) | | | ACAGAGAAGGTCTGGGGGAC′] |
| 900 | NM_000053.3(ATP7B): | 540 | ATP7B | [′GCCTTCACTGTCCTTGTCTTTCAGCYCCTCG |
| | c.2123T > C (p.Leu708Pro) | | | GTGGGTGGTACTTCTACGTT′] |
| 901 | NM_000520.4(HEXA): c.1453T > | 3073 | HEXA | [′AGCAGGGGCTGTTGCCGAAAGGCTGYGGAG |
| | C (p.Trp485Arg) | | | CAACAAGTTGACATCTGACCT′] |
| 902 | NM_000520.4(HEXA): c.632T > C | 3073 | HEXA | [′TGGCATCTGGTAGATGATCCTTCCTYCCCAT |
| | (p.Phe211Ser) | | | ATGAGAGCTTCACTTTTCCA′] |
| 903 | NM_000520.4(HEXA): c.538T > C | 3073 | HEXA | [′CTTGCTGTTGGATACATCTCGCCATYACCTG |
| | (p.Tyr180His) | | | CCACTCTCTAGCATCCTGGA′] |
| 904 | NM_016335.4(PRODH): | 5625 | PRODH | [′CTGGGCCAGGTATGCGCCCCGCACCRGCTTG |
| | c.1322T > C (p.Leu441Pro) | | | GCCCCAAAACACCAGCCCTC′] |
| 905 | NM_000152.3(GAA): c.953T > C | 2548 | GAA | [′GTGTTCCTGCTAAACAGCAATGCCAYGGGT |
| | (p.Met318Thr) | | | AAGCTGCCCGCCGCCCAGCGC′] |
| 906 | NM_012213.2(MLYCD): | 23417 | MLYCD | [′GCGGCCGGCGCCCTGGAGCGGGCCAYGGAC |
| | c.119T > C (p.Met40Thr) | | | GAGCTGCTGCGCCGCGCGGTG′] |
| 907 | NM_012464.4(TLL1): c.713T > C | 7092 | TLL1 | [′AAGAACTGTGATAAATTTGGGATTGYTGTTC |
| | (p.Val238Ala) | | | ATGAATTGGGTCATGTGATA′] |
| 908 | NM_000112.3(SLC26A2): | 1836 | SLC26A2 | [′CCTGCAGCGGCCCGGACCCGAGAGGYGAGA |
| | c.−26 + 2T > C | | | AGAGGGAAGCGGACCAGGGAA′] |
| 909 | NM_138691.2(TMC1): c.1543T > | 117531 | TMC1 | [′CCCTGCAGATGTACCTCGAGGACCTYGCTGG |
| | C (p.Cys515Arg) | | | GAAACAATGGTGGGACAGGT′] |
| 910 | NM_138694.3(PKHD1): | 5314 | PKHD1 | [′GTTGTCCTACAAGGAGAGGAGCCCAYTGAA |
| | c.10658T > C (p.Ile3553Thr) | | | ATACGCTCAGGTGTTTCCATT′] |
| 911 | NM_001034116.1(EIF2B4): | 8890 | EIF2B4 | [′TGCAGATGACCCTGATGATCTGCAAYGTAA |
| | c.1393T > C (p.Cys465Arg) | | | GCGGGGAGAACATGTTGCGCT′] |
| 912 | NM_001034116.1(EIF2B4): | 8890 | EIF2B4 | [′ATCCCTACGGTTGTTGAATCTAGTCYATGAT |
| | c.1465T > C (p.Tyr489His) | | | GTGACTCCCCCAGAGCTTGT′] |
| 913 | NM_018960.4(GNMT): c.149T > | 27232 | GNMT | [′ACCGCCGAGTACAAGGCATGGCTGCYTGGG |
| | C (p.Leu50Pro) | | | CTGCTGCGCCAGCACGGCTGC′] |
| 914 | NM_181457.3(PAX3): c.268T > C | 5077 | PAX3 | [′CTGCGTCTCCAAGATCCTGTGCAGGYACCAG |
| | (p.Tyr90His) | | | GAGACTGGCTCCATACGTCC′] |
| 915 | NM_024301.4(FKRP): c.899T > C | 79147 | FKRP | [′GAGACCACGCGCTGCTTCGGAACCGYGGTG |
| | (p.Val300Ala) | | | GGCGACACGCCCGCCTACCTC′] |
| 916 | NM_021020.3(LZTS1): c.85T > C | 11178 | LZTS1 | [′TTCGCAGTACAAGCTGCGCAAGTCCYCCCAC |
| | (p.Ser29Pro) | | | CTCAAGAAGCTCAACCGGTA′] |
| 917 | NM_005857.4(ZMPSTE24): | 10269 | ZMPSTE24 | [′TGTACTAGGCCATGAACTGGGGCACYGGAA |
| | c.1018T > C (p.Trp340Arg) | | | GTTGGGACATACAGTCAAAAA′] |
| 918 | NM_017838.3(NHP2): c.415T > C | 55651 | NHP2 | [′GCCCCATGAGGAGTACCAGGAGGCTYACGA |
| | (p.Tyr139His) | | | TGAGTGCCTGGAGGAGGTGCA′] |
| 919 | NM_000157.3(GBA): c.1448T > C | 2629 | GBA | [′GGGATGCATCAGTGCCACTGCGTCCVGGTC |
| | (p.Leu483Pro) | | | GTTCTTCTGACTGGCAACCAG′] |
| 920 | NM_001005741.2(GBA): | 2629 | GBA | [′CATCTACCACCAGACCTGGGCCAGAYACTTT |
| | c.751T > C (p.Tyr251His) | | | GTGAAGTAAGGGATCAGCAA′] |
| 921 | NM_001243133.1(NLRP3): | 114548 | NLRP3 | [′AAATTCGAAAAGGGGTATTTGATTTYTGTTG |
| | c.1718T > C (p.Phe573Ser) | | | TACGTTTCCTCTTTGGCCTG′] |
| 922 | NM_001243133.1(NLRP3): | 114548 | NLRP3 | [′GGCTTCGATGAGCTGCAAGGTGCCTYTGAC |
| | c.926T > C (p.Phe309Ser) | | | GAGCACATAGGACCGCTCTGC′] |
| 923 | NM_001243133.1(NLRP3): | 114548 | NLRP3 | [′CTCATCACCACGAGACCTGTGGCCCYGGAG |
| | c.1058T > C (p.Leu353Pro) | | | AAACTGCAGCACTTGCTGGAC′] |
| 924 | NM_057176.2(BSND): c.35T > C | 7809 | BSND | [′GAGAAGACCTTCCGGATCGGCTTCAYTGTGC |
| | (p.Ile12Thr) | | | TGGGGCTTTTCCTGCTGGCC′] |
| 925 | NM_001195794.1(CLRN1): | 7401 | CLRN1 | [′TCTGTTGCAGGCTCCTGTGGCTGTCYTGTCA |
| | c.488T > C (p.Leu163Pro) | | | TGATATTGTTTGCCTCTGAA′] |
| 926 | NM_175073.2(APTX): c.668T > C | 54840 | APTX | [′GCTGTGGCCAGGGAACACCTTGAACYCCTT |
| | (p.Leu223Pro) | | | AAGCATATGCACACTGTGGGG′] |
| 927 | NM_020365.4(EIF2B3): | 8891 | EIF2B3 | [′CCACCAGTCCATTCGTCAGCCCAGAYTGTCA |
| | c.1037T > C (p.Ile346Thr) | | | GCAAACACCTGGTAAGTGCT′] |
| 928 | NM_031433.3(MFRP): c.545T > C | −1 | — | [′ATCCAGGTGGCCACAGACCATGCAAYACAG |
| | (p.Ile182Thr) | | | CTCAAGATCGAAGCCCTCAGC′] |
| 929 | NM_016180.4(SLC45A2): | 51151 | SLC45A2 | [′AGTGCACACAACTCCACAGAGTTTCYCATCT |
| | c.1082T > C (p.Leu361Pro) | | | ACGAAAGAGGAGTCGAGGTT′] |
| 930 | NM_006005.3(WFS1): c.2486T > | 7466 | WFS1 | [′AGCCTCATCGAGTTCAGCACCATCCYGGAG |
| | C (p.Leu829Pro) | | | GGCCGCCTGGGCAGCAAGTGG′] |
| 931 | NM_024960.4(PANK2): c.178T > | 80025 | PANK2 | [′GATCAAAGGAATTTTATACATTGACYCAGTC |
| | C (p.Ser60Pro) | | | GGATTCAATGGACGGTCACA′] |
| 932 | NM_024960.4(PANK2): c.437T > | 80025 | PANK2 | [′ACCACTTTTGAAGAAGCTCTTGAAAYGGCAT |
| | C (p.Met146Thr) | | | CTCGTGGAGATAGCACCAAA′] |
| 933 | NM_020427.2(SLURP1): c.43T > | 57152 | SLURP1 | [′TGTGCAGCTGCTGCTCGTGGCAGCCYGGAG |
| | C (p.Trp15Arg) | | | CATGGGCTGTGGTGAGTGGGC′] |
| 934 | NM_020427.2(SLURP1): | 57152 | SLURP1 | [′GGTGACCCGCTCCTGCTCCAGCTCCYGTGTG |
| | c.229T > C (p.Cys77Arg) | | | GCCACCGACCCCGACAGCAT′] |
| 935 | NM_021830.4(C10orf2): | 56652 | C10orf2 | [′CTTCGGGAGGAGGTGCTAGGAGAACBGTCA |
| | c.1142T > C (p.Leu381Pro) | | | AATGTGGAGCAAGCAGCTGGC′] |
| 936 | NM_001033855.2(DCLREIC): | 64421 | DCLREIC | [′CCGGACTCTGGGATCGGCGGCGCTAYGAGT |
| | c.2T > C (p.Met1Thr) | | | TCTTTCGAGGGGCAGATGGCC′] |
| 937 | NM_153741.1(DPM3): c.254T > C | 54344 | DPM3 | [′CAGATACAGGAGGCCCGAGCCGACTYAGCC |
| | (p.Leu85Ser) | | | CGCAGGGGGCTGCGCTTCTGA′] |
| 938 | NM_000441.1(SLC26A4): | 5172 | SLC26A4 | [′GCCTTCCAAGTGCTGGTCTCACAGCYAAAG |
| | c.707T > C (p.Leu236Pro) | | | ATTGTCCTCAATGTTTCAACC′] |
| 939 | NM_000441.1(SLC26A4): | 5172 | SLC26A4 | [′TGGAAGCATCCCTAGCACAGATATCYACAA |
| | Tc.1588 > C (p.Tyr530His) | | | AAGTACCAAGAATTACAAAAA′] |
| 940 | NM_000441.1(SLC26A4): | −1 | — | [′CTTTCCCTTCGACCAAGGTGTCTGTYGCTCC |
| | c.−103T > C | | | GTAAATAAAACGTCCCACTG′] |
| 941 | NM_000441.1(SLC26A4): | 5172 | SLC26A4 | [′ATTTTTCACTTAAAAACTCACTAGGHTTTTG |
| | c.1003T > C (p.Phe335Leu) | | | CCTCCTGAACTTCCACCTGT′] |
| 942 | NM_022458.3(LMBR1): | 64327 | LMBR1 | [′AACAAAGATTTTTTTAATATGTTTCYATCCT |
| | c.423 + 4842T > C | | | GTGTCACAGTTTGAAATTGT′] |
| 943 | NM_022458.3(LMBR1): | 64327 | LMBR1 | [′AAAGCTGAGCAACATGACAGCACAAYAGAG |
| | c.423 + 4808T > C | | | GAGGAACAAAGATTTTTTTAA′] |
| 944 | NM_145693.2(LPIN1): | 23175 | LPIN1 | [′GCGACCACCGGGAGATCACGAAAGGYACCG |
| | c.1441 + 2T > C | | | CGGGCCTCGCGCGGGCGCCCT′] |
| 945 | NM_022124.5(CDH23): | 64072 | CDH23 | [′AGTGGCTGCAATGCACGCCTCACCTYCAAC |
| | c.5663T > C (p.Phe1888Ser) | | | ATCACTGCGGGCAACCGCGAG′] |
| 946 | NM_153212.2(GJB4): c.409T > C | 127534 | GJB4 | [′GTGGACGTACTTGCTGAGCCTCATCYTCAAG |
| | (p.Phe137Leu) | | | GCCGCCGTGGATGCTGGCTT′] |
| 947 | NM_021044.2(DHH): c.2T > C | 50846 | DHH | [′TTTTGGCCGAGGTCCGCTGTATCCAYGGCTC |
| | (p.Met1Thr) | | | TCCTGACCAATCTACTGCCC′] |
| 948 | NM_021044.2(DHH): c.485T > C | 50846 | DHH | [′CGCGACCGCAACAAGTATGGGTTGCYGGCG |
| | (p.Leu162Pro) | | | CGCCTCGCAGTGGAAGCCGGC′] |
| 949 | NM_020638.2(FGF23): c.287T > | 8074 | FGF23 | [′GTGATGAGCAGAAGATACCTCTGCAYGGAT |
| | C (p.Met96Thr) | | | TTCAGAGGCAACATTTTTGGA′] |
| 950 | NM_022041.3(GAN): c.1268T > C | 8139 | GAN | [′TGCTATGCAGCTATGAAAAAGAAAAYCTAC |
| | (p.Ile423Thr) | | | GCCATGGGTGGAGGCTCCTAC′] |
| 951 | NM_015665.5(AAAS): c.787T > C | 8086 | AAAS | [′CAGTGGGGGGCGGCTGCTCTCAGCTYCACC |
| | (p.Ser263Pro) | | | CGTGGATGCTGCTATCCGGGT′] |
| 952 | NM_021615.4(CHST6): c.827T > | 4166 | CHST6 | [′TACCGCCTGGTGCGCTTCGAGGACCYGGCG |
| | C (p.Leu276Pro) | | | CGGGAGCCGCTGGCAGAAATC′] |
| 953 | NM_000368.4(TSC1): c.539T > C | 7248 | TSC1 | [′GTGGCGGAAGTCTATCTCGTCCATCYCCATG |
| | (p.Leu180Pro) | | | CCAGTGTGTACGCACTCTTT′] |
| 954 | NM_020661.2(AICDA): c.238T > | 57379 | AICDA | [′CCCTGGCCGCTGCTACCGCGTCACCYGGTTC |
| | C (p.Trp80Arg) | | | ACCTCCTGGAGCCCCTGCTA′] |
| 955 | NM_020661.2(AICDA): c.317T > | 57379 | AICDA | [′CTGCGAGGGAACCCCAACCTCAGTCYGAGG |
| | C (p.Leu106Pro) | | | ATCTTCACCGCGCGCCTCTAC′] |
| 956 | NM_020661.2(AICDA): c.452T > | 57379 | AICDA | [′GATTATTTTTACTGCTGGAATACTTYTGTAG |
| | C (p.Phe151Ser) | | | AAAACCACGAAAGAACTTTC′] |
| 957 | NM_020632.2(ATP6V0A4): | 50617 | ATP6V0A4 | [′ATCATTCTGCAATTTATCCCTGAGAYGATTT |
| | c.1739T > C (p.Met580Thr) | | | TTATCCTGTGTCTGTTTGGA′] |
| 958 | NM_054027.4(ANKH): c.143T > | 56172 | ANKH | [′GCTGTCAAGGAGGATGCAGTCGAGAYGCTG |
| | C (p.Met48Thr) | | | GCCAGCTACGGGCTGGCGTAC′] |
| 959 | NM_054027.4(ANKH): c.1015T > | 56172 | ANKH | [′TGTTTGATGTCTTTCTCCCCAGCTCYGTTTCG |
| | C (p.Cys339Arg) | | | TGATGTTTTGGACACCCAA′] |
| 960 | NM_054027.4(ANKH): c.1172T > | −1 | — | [′GTGAGGGCGCATCTCACCGGGTGGCYGATG |
| | C (p.Leu391Pro) | | | ACACTGAAGAAAACCTTCGTC′] |
| 961 | NM_016373.3(WWOX): c.872T > | 51741 | WWOX | [′ACAAAAAACGACTATTGGGCGATGCYGGCT |
| | C (p.Leu291Pro) | | | TATAACAGGTCCAAGCTCTGC′] |
| 962 | NM_021102.3(SPINT2): | 10653 | SPINT2 | [′CAGCGGATTCCTCTGTCCCAAGTGGYAGGTT |
| | c.337 + 2T > C | | | CTTAAAGAGACCCGCGATGG′] |
| 963 | NM_014588.5(VSX1): c.50T > C | 30813 | VSX1 | [′GACGGGCGCACTAGCAGCAGGGCGCYGGTG |
| | (p.Leu17Pro) | | | CCTGGCGGTTCCCCTAGGGGC′] |
| 964 | NM_006946.2(SPTBN2): | 6712 | SPTBN2 | [′GAAAAGGAACTGGGACTTACCAAGCYGCTG |
| | c.758T > C (p.Leu253Pro) | | | GATCCCGAAGGTGGGGCCAGA′] |
| 965 | NM_012452.2(TNFRSF13B): | 23495 | TNFRSF13B | [′GCACCCTAAGCAATGTGCATACTTCYGTGAG |
| | c.310T > C (p.Cys104Arg) | | | AACAAGCTCAGGAGCCCAGT′] |
| 966 | NM_170784.2(MKKS): c.830T > | 8195 | MKKS | [′GAAAATGCAGTCTTGGACCAGCTGCYTAAC |
| | C (p.Leu277Pro) | | | CTAGGAAGGCAGCTAATCAGT′] |
| 967 | NM_015717.4(CD207): c.790T > | 50489 | CD207 | [′AATGGCGTGTCATCCACCCAGGACCRGTCCC |
| | C (p.Trp264Arg) | | | CTTCCATCCCTGCTTTAGTC′] |
| 968 | NM_153717.2(EVC): c.919T > C | 2121 | EVC | [′TGTGGAAAAGAAGGAGAGAGAATACYCTGA |
| | (p.Ser307Pro) | | | ACAGCTAATCGATAATGTGCG′] |
| 969 | NM_014384.2(ACAD8): c.455T > | 27034 | ACAD8 | [′AAATTTTGCCCACCGCTCTGTACCAYGGAGA |
| | C (p.Met152Thr) | | | AGTTTGCTTCCTACTGCCTC′] |
| 970 | NM_004924.4(ACTN4): c.784T > | 81 | ACTN4 | [′CGAGAAGGCCATAATGACCTATGTGYCCAG |
| | C (p.Ser262Pro) | | | CTTCTACCATGCCTTTTCAGG′] |
| 971 | NM_005097.3(LGI1): c.136T > C | 9211 | LGI1 | [′AGCGAAGCCAAAATGCCCTGCCGTGYGTAC |
| | (p.Cys46Arg) | | | TTGTACCAAAGATAATGCTTT′] |
| 972 | NM_005097.3(LGI1): c.695T > C | 9211 | LGI1 | [′GCAGAATTTGCAAAGTCTCAAGACCYGCCTT |
| | (p.Leu232Pro) | | | ATCAATCATTGTCCATAGAC′] |
| 973 | NM_006329.3(FBLN5): c.679T > | 10516 | FBLN5 | [′GCAAACCTGCGTCAACACCTACGGCYCTTTC |
| | C (p.Ser227Pro) | | | ATCTGCCGCTGTGACCCAGG′] |
| 974 | NM_006329.3(FBLN5): c.506T > | 10516 | FBLN5 | [′TTGCTTGCATTTCTGTTTCCAGACAYTGATG |
| | C (p.Ile169Thr) | | | AATGTCGCTATGGTTACTGC′] |
| 975 | NM_013339.3(ALG6): c.1432T > | 29929 | ALG6 | [′GTTTTCTGTATTGGTGTGTTTTGTAYCTTGCT |
| | C (p.Ser478Pro) | | | TGAACTTCCTGTTCTTCTT′] |
| 976 | NM_006899.3(IDH3B): c.395T > | 3420 | IDH3B | [′GAGCTAGCCTCCTATGATATGCGGCYGAGG |
| | C (p.Leu132Pro) | | | TAGGTGGTCTGGGTGGGGTGA′] |
| 977 | NM_014363.5(SACS): c.5836T > | 26278 | SACS | [′AATGGATTATACTTACTATGCAGTAYGGCCC |
| | C (p.Trp1946Arg) | | | GATCCTGATTTAGTTCATGA′] |
| 978 | NM_014363.5(SACS): c.9742T > | 26278 | SACS | [′AAGTGAGTCTTGGCTTAAGAATGCAYGGCA |
| | C (p.Trp3248Arg) | | | TTTTATTAGTGAATCTGTAAG′] |
| 979 | NM_014363.5(SACS): c.3161T > | 26278 | SACS | [′CAGATGGTATCAGCTGGTGAACTCTYTGACC |
| | C (p.Phe 1054Ser) | | | CTGATATAGAAGTACTAAAG′] |
| 980 | NM_014324.5(AMACR): | −1 | — | [′GAGCCGCTTGGGCCGGGGCAAGCGCYCGCT |
| | c.154T > C (p.Ser52Pro) | | | AGTGCTGGACCTGAAGCAGCC′] |
| 981 | NM_014324.5(AMACR): | −1 | — | [′AATCCAAGGCTTATTTATGCCAGGCYGAGTG |
| | c.320T > C (p.Leu107Pro) | | | GATTTGGCCAGTCAGGAAGC′] |
| 982 | NM_001040108.1(MLH3): | 27030 | MLH3 | [′GACAGAGGAACAAAGGAGACTCTTAYGGTC |
| | 6c.382T > C (p.Trp1276Arg) | | | AGTACCACCATGAGAATGTGA′] |
| 983 | NM_014336.4(AIPL1): c.715T > C | 23746 | AIPL1 | [′TACTCTGATCCTCAACTACTGCCAGYGCCTG |
| | (p.Cys239Arg) | | | CTGAAGAAGGAGGAGTACTA′] |
| 984 | NM_001001486.1(ATP2C1): | 27032 | ATP2C1 | [′CTCTCATTTGCTTTAGCCAGTCGTCYGGGAT |
| | c.1751T > C (p.Leu584Pro) | | | TGTATTCCAAAACTTCCCAG′] |
| 985 | NM_007194.3(CHEK2): c.470T > | 11200 | CHEK2 | [′GAAGTGGGTCCTAAAAACTCTTACAYTGCAT |
| | C (p.Ile157Thr) | | | ACATAGAAGATCACAGTGGC′] |
| 986 | NM_007255.2(B4GALT7): | 11285 | B4GALT7 | [′ACCTATGTCGGCGGCATCCTGCTGCYCTCCA |
| | c.617T > C (p.Leu206Pro) | | | AGCAGCACTACCGGCTGGTG′] |
| 987 | NM_000030.2(AGXT): c.613T > C | 189 | AGXT | [′ATCTACAGGCATCGACATCCTGTACYCGGGC |
| | (p.Ser205Pro) | | | TCCCAGAAGGCCCTGAACGC′] |
| 988 | NM_000030.2(AGXT): c.731T > C | 189 | AGXT | [′AAGCCCTTCTCCTTCTACCTGGACAYCAAGT |
| | (p.Ile244Thr) | | | GGCTGGCCAACTTCTGGGGC′] |
| 989 | NM_201253.2(CRB1): c.3122T > | 23418 | CRB1 | [′ACATGGCACGAAGTGACCCTTTCCAYGACA |
| | C (p.Met1041Thr) | | | GACCCACTGTCCCAGACCTCC′] |
| 990 | NM_201253.2(CRB1): c.3541T > | 23418 | CRB1 | [′ACACTGTGAACTCAACATCGATGAAYGCTTT |
| | C (p.Cys1181Arg) | | | TCAAACCCCTGTATCCATGG′] |
| 991 | NM_005094.3(SLC27A4): | 10999 | SLC27A4 | [′AGATAAACTGTTCTACATCTACACAYCCGGC |
| | c.739T > C (p.Ser247Pro) | | | ACCACAGGGCTGCCCAAGGC′] |
| 992 | NM_032551.4(KISS1R): c.443T > | 84634 | KISS1R | [′CGCTGGTACGTGACGGTGTTCCCGTYGCGCG |
| | C (p.Leu148Ser) | | | CCCTGCACCGCCGCACGCCC′] |
| 993 | NM_032551.4(KISS1R): c.305T > | 84634 | KISS1R | [′TGCTGCGTCCCCTTCACGGCCCTGCYGTACC |
| | C (p.Leu102Pro) | | | CGCTGCCCGGCTGGGTGCTG′] |
| 994 | NM_014270.4(SLC7A9): | 11136 | SLC7A9 | [′ATCTCCATCATCGTGGGCACCATCAYTGGCT |
| | c.131T > C (p.Ile44Thr) | | | CTGGGATCTTCGTTTCCCCC′] |
| 995 | NM_003332.3(TYROBP): c.2T > | 7305 | TYROBP | [′TGGTGTCCAGCAGCATCCGGCTTCAYGGGG |
| | C (p.Met1Thr) | | | GGACTTGAACCCTGCAGCAGG′] |
| 996 | NM_000843.3(GRM6): c.1214T > | −1 | — | [′CAGGAGGGCAAGGTGCAGTTTGTGAYTGAT |
| | C (p.Ile405Thr) | | | GCGGTGTACGCCATTGCCCAC′] |
| 997 | NM_022336.3(EDAR): c.259T > C | 10913 | EDAR | [′GTTTTCCAAAGGAGGCTACCAGATAYGCAG |
| | (p.Cys87Arg) | | | GCGTCACAAAGACTGTGAGGG′] |
| 998 | NM_003835.3(RGS9): c.895T > C | 8787 | RGS9 | [′CCCAACCAAGATGCGAGTGGAACGAYGGGC |
| | (p.Trp299Arg) | | | CTTCAACTTCAGCGAATTGAT′] |
| 999 | NM_004870.3(MPDU1): c.356T > | 9526 | MPDU1 | [′CTCCAGACGATCACCATCTGCTTCCYGGTCA |
| | C (p.Leu119Pro) | | | TGCACTACAGAGGACAGACT′] |
| 1000 | NM_004870.3(MPDU1): c.2T > C | 9526 | MPDU1 | [′ACTGGCGGAAGCTAGCTTTGCAATAYGGCG |
| | (p.Met1Thr) | | | GCCGAGGCGGACGGACCGCTT′] |
| 1001 | NM_004870.3(MPDU1): c.221T > | 9526 | MPDU1 | [′CTGGGAGCCAAGAGTGCTGAAGGGTYGAGT |
| | C (p.Leu74Ser) | | | CTCCAGTCTGTAATGCTGGAG′] |
| 1002 | NM_000334.4(SCN4A): | 6329 | SCN4A | [′ACGCTGAACATGCTCATCAAGATCAYTGGC |
| | c.2078T > C (p.Ile693Thr) | | | AATTCAGTGGGGGCGCTGGGT′] |
| 1003 | NM_006580.3(CLDN16): | 10686 | CLDN16 | [′GCTGGGTTTGGATTTCTCACCCTGCYCCTTG |
| | c.500T > C (p.Leu167Pro) | | | GTCTTGACTGCGTGAAATTC′] |
| 1004 | NM_006580.3(CLDN16): | 10686 | CLDN16 | [′ATATGCCCTGGTCTTCCAGTGAAGCYGGTGG |
| | c.434T > C (p.Leu145Pro) | | | TAACTCGAGCGTTGATGATT′] |
| 1005 | NM_003907.2(EIF2B5): | 8893 | EIF2B5 | [′CTTTCTTCCATAGCTGCTAAAGGCCYGGAGC |
| | c.1882T > C (p.Trp628Arg) | | | CCTGTTTTTAGGAACTACAT′] |
| 1006 | NM_183235.2(RAB27A): | 5873 | RAB27A | [′TATTGTGAAAACCCAGATATAGTGCYGTGTG |
| | c.389T > C (p.Leu130Pro) | | | GAAACAAGAGTGATCTGGAG′] |
| 1007 | NM_001128227.2(GNE): | 10020 | GNE | [′CCCGCCCTGCTGGGTGCTGCCAGCAYGGTTC |
| | c.2228T > C (p.Met743Thr) | | | TGGACTACACAACACGCAGG′] |
| 1008 | NM_004273.4(CHST3): c.776T > | 9469 | CHST3 | [′CGCTGCGGCCCCCTCAACGTGACGCYGGCC |
| | C (p.Leu259Pro) | | | GCAGAGGCCTGCCGCCGCAAG′] |
| 1009 | NM_004273.4(CHST3): c.920T > | 9469 | CHST3 | [′CTGGTGCGCGACCCCCGGGCCGTGCYGGCC |
| | C (p.Leu307Pro) | | | TCGCGCATGGTGGCCTTCGCC′] |
| 1010 | NM_004273.4(CHST3): c.857T > | 9469 | CHST3 | [′CGGCAGCTGGAGTTCCTGCAGCCGCYGGCC |
| | C (p.Leu286Pro) | | | GAGGACCCCCGCCTGGACCTG′] |
| 1011 | NM_172201.1(KCNE2): c.161T > | 9992 | KCNE2 | [′TTCTACTATGTCATCCTGTACCTCAYGGTGA |
| | C (p.Met54Thr) | | | TGATTGGAATGTTCTCTTTC′] |
| 1012 | NM_172201.1(KCNE2): c.170T > | 9992 | KCNE2 | [′GTCATCCTGTACCTCATGGTGATGAYTGGAA |
| | C (p.Ile57Thr) | | | TGTTCTCTTTCATCATCGTG′] |
| 1013 | NM_172201.1(KCNE2): c.178T > | 9992 | KCNE2 | [′GTACCTCATGGTGATGATTGGAATGHTCTCT |
| | C (p.Phe60Leu) | | | TTCATCATCGTGGCCATCCT′] |
| 1014 | NM_001139.2(ALOX12B) | 242 | ALOX12B | [′TCATCCTCCTTGCTCTCCCCACAGCYCCTCA |
| | c.1277T > C (p.Leu426Pro) | | | TCCCCCATACCCGATACACC′] |
| 1015 | NM_003640.3(IKBKAP): | 8518 | IKBKAP | [′ATTCGGAAGTGGTTGGACAAGTAAGYGCCA |
| | c.2204 + 6T > C | | | TTGTACTGTTTGCGACTAGTT′] |
| 1016 | NM_005413.3(SIX3): c.749T > C | 6496 | SIX3 | [′GCCACCGGCCTCACTCCCACACAAGYAGGC |
| | (p.Val250Ala) | | | AACTGGTTTAAGAACCGGCGG′] |
| 1017 | NM_004820.3(CYP7B1): | 9420 | CYP7B1 | [′TTTATTAGTGAGCTAAGAGATGATTYTTTAA |
| | c.647T > C (p.Phe216Ser) | | | AATTTGATGACAAGTTTGCA′] |
| 1018 | NM_194248.2(OTOF): c.3032T > | 9381 | OTOF | [′CTGTGTCCCACCTGGGACCAGATGCYGGTGT |
| | C (p.Leu1011Pro) | | | TCGACAACCTGGAGCTCTAT′] |
| 1019 | NM_194248.2(OTOF): c.1544T > | 9381 | OTOF | [′GACGTGGCCATCGGCACCCACTTCAYTGACC |
| | C (p.Ile515Thr) | | | TGCGCAAGATTTCTAATGAC′] |
| 1020 | NM_004737.4(LARGE): | 9215 | LARGE | [′GATGCTGGAGGCCATCTGCAAGCACYGGGA |
| | c.1483T > C (p.Trp495Arg) | | | GGGGCCCATCAGCCTGGCCCT′] |
| 1021 | NM_002420.5(TRPM1): c.296T > | 4308 | TRPM1 | [′GAGGCCTCCATGCACAGATATTAAGRGCTTG |
| | C (p.Leu99Pro) | | | GGGAGTTCCAGCTGCCAATC′] |
| 1022 | NM_004700.3(KCNQ4): c.842T > | 9132 | KCNQ4 | [′ACCTGTTTGTGTCTCCAGATTACATYGACAA |
| | C (p.Leu281Ser) | | | CCATCGGCTATGGTGACAAG′] |
| 1023 | NM_003839.3(TNFRSF11A): | 8792 | TNFRSF11A | [′ACTGAACCACCTTTTCCCCCACAGCYGTACC |
| | c.523T > C (p.Cys175Arg) | | | TTCCTTGGAAAGAGAGTAGA′] |
| 1024 | NM_030761.4(WNT4): c.35T > C | 54361 | WNT4 | [′CGCTCGTGCCTGCGTTCGCTGCGCCYCCTCG |
| | (p.Leul2Pro) | | | TCTTCGCCGTCTTCTCAGCC′] |
| 1025 | NM_000050.4(ASS1): c.535T > C | 445 | ASS1 | [′CATCCCGGTCACTCCCAAGAACCCGYGGAG |
| | (p.Trp179Arg) | | | CATGGATGAGAACCTCATGCA′] |
| 1026 | NM_002977.3(SCN9A): | −1 | — | [′ACATTGAACATGCTGATTAAGATCAYTGGTA |
| | c.2543T > C (p.Ile848Thr) | | | ACTCAGTAGGGGCTCTAGGT′] |
| 1027 | NM_002977.3(SCN9A): | −1 | — | [′TTTCTTTACCTTGGAGGTCAAGACAYCTTTA |
| | c.4382T > C (p.Ile1461Thr) | | | TGACAGAAGAACAGAAGAAA′] |
| 1028 | NM_002977.3(SCN9A): c.647T > | 6335 | SCN9A | [′GGCAATGTTTCAGCTCTTCGAACTTYCAGAG |
| | C (p.Phe216Ser) | | | TATTGAGAGCTTTGAAAACT′] |
| 1029 | NM_003880.3(WISP3): c.232T > | 8838 | WISP3 | [′AGATGGCTGTGGATGCTGTAAAATCYGTGC |
| | C (p.Cys78Arg) | | | CAAGCAACCAGGGGAAATCTG′] |
| 1030 | NM_003880.3(WISP3): c.1000T > | 8838 | WISP3 | [′TAAATGGAAGATGCTGTGGATTACAYCTTGT |
| | C (p.Ser334Pro) | | | GTGTGTCAGAGAAACTGCAG′] |
| 1031 | NM_001457.3(FLNB): c.703T > C | 2317 | FLNB | [′CGAGCACTCAGTTATGACTTACCTGYCCCAG |
| | (p.Ser235Pro) | | | TTCCCCAAAGCCAAGCTCAA′] |
| 1032- | NM_003060.3(SLC22A5): | 6584 | SLC22A5 | [′GATTTGTTACTGACAAGGCCTAGGGNAAGTT |
| 1036 | c.1051T > C (p.Trp351Arg) | | | TTCACAGCCTAAAACACAGT′, |
| | | | | ′TACAAGACCTAAGTTCCAAGAAGCARCAGTC |
| | | | | CCACAACATTCTGGATCTGC′, |
| | | | | ′GGTCACCATCATGTCCATAATGCTGYGGTAT |
| | | | | GTAAAAGAGACCTGCCTGAG′, |
| | | | | ′ATGCCTCAGACAAAATTCAAAGCCTRTGTCA |
| | | | | TCAGAGAGTGAAAAGGATAT′, |
| | | | | ′GGCTGTTGTGGGAAATATGGACTCTYGTGGG |
| | | | | GAATCTCTCCAGATCTTAAG′] |
| 1037 | NM_000369.2(TSHR): c. 1891T > | 7253 | TSHR | [′TGCCAAGAGGATGGCTGTGTTGATCYTCACC |
| | C (p.Phe631Leu) | | | GACTTCATATGCATGGCCCC′] |
| 1038 | NM_000369.2(TSHR): c. 1358T > | 7253 | TSHR | [′AAACTGAACGTCCCCCGCTTTCTCAYGTGCA |
| | C (p.Met453Thr) | | | ACCTGGCCTTTGCGGATTTC′] |
| 1039 | NM_000369.2(TSHR): c.1526T > | 7253 | TSHR | [′ACTGTCTTTGCAAGCGAGTTATCGGYGTATA |
| | C (p.Val509Ala) | | | CGCTGACGGTCATCACCCTG′] |
| 1040 | NM_000369.2(TSHR): c. 1798T > | 7253 | TSHR | [′AGTTGCCTTCGTCATCGTCTGCTGCYGTTAT |
| | C (p.Cys600Arg) | | | GTGAAGATCTACATCACAGT′] |
| 1041 | NM_000369.2(TSHR): c.1400T > | 7253 | TSHR | [′GCGGATTTCTGCATGGGGATGTACCYGCTCC |
| | C (p.Leu467Pro) | | | TCATCGCCTCTGTAGACCTC′] |
| 1042 | NM_001003722.1(GLE1): | 2733 | GLE1 | [′CAGAAATGTTTGCAACACAAGGACAYTCCT |
| | c.2051T > C (p.Ile684Thr) | | | GTCCCCAAGGGCTTTCTGACT′] |
| 1043 | NM_024009.2(GJB3): c.101T > C | 2707 | GJB3 | [′TCCGTGGTGTTCGTCTTCCGGGTGCYGGTAT |
| | (p.Leu34Pro) | | | ACGTGGTGGCTGCAGAGCGC′] |
| 1044 | NM_001080463.1(DYNC2H1): | 79659 | DYNC2H1 | [′GATTTGCTCAGAGTAGCTGATACAAYTGTAG |
| | c.3719T > C (p.Ile1240Thr) | | | CCAAAGCTGCCGACCTTAAA′] |
| 1045 | NM_018129.3(PNPO): c.784T > C | 55163 | PNPO | [′CTGGCTCTATGAGAGACTTGCACCTYAACTC |
| | (p.Ter262Gln) | | | TGGGACCTGCTGGCCCAGAG′] |
| 1046 | NM_003722.4(TP63): c.1033T > C | 8626 | TP63 | [′CCGACGCTGCTTTGAGGCCCGGATCYGTGCT |
| | (p.Cys345Arg) | | | TGCCCAGGAAGAGACAGGAA′] |
| 1047 | NM_003722.4(TP63): c.1646T > C | 8626 | TP63 | [′CCTCCGTATCCCACAGATTGCAGCAYTGTCA |
| | (p.Ile549Thr) | | | GGTGAGTCCACAGCATGTGC′] |
| 1048 | NM_003722.4(TP63): c.1738T > C | 8626 | TP63 | [′CACCATCTATCAGATTGAGCATTACBCCATG |
| | (p.Ser580Pro) | | | GATGTAAGTAACTGTTAGAC′] |
| 1049 | NM_004086.2(COCH): c.349T > C | −1 | — | [′CATCCAGTCTCAAATGCTTTCTAGAYGGTCT |
| | (p.Trp117Arg) | | | GCTTCTTTCACAGTAACTAG′] |
| 1050 | NM_004086.2(COCH): c.1535T > | −1 | — | [′GCACCTCTGGATGACCTGAAAGATAYGGCT |
| | C (p.Met512Thr) | | | TCTAAACCGAAGGAGTCTCAT′] |
| 1051 | NM_006412.3(AGPAT2): | 10555 | AGPAT2 | [′ACAGGAACAGTCACAGTGCAGGTGCYGGAA |
| | c.683T > C (p.Leu228Pro) | | | GCCATCCCCACCAGCGGCCTC′] |
| 1052 | NM_003659.3(AGPS): c.1406T > | 8540 | AGPS | [′AATCAGCTAAGTGTAGCCACATTACYGTTTG |
| | C (p.Leu469Pro) | | | AGGGGGATCGTGAGAAGGTT′] |
| 1053 | NM_004550.4(NDUFS2): | 4720 | NDUFS2 | [′GGGAGAGTTTGGGGTGTACCTGGTGYCTGA |
| | c.1237T > C (p.Ser413Pro) | | | TGGCAGCAGCCGCCCTTATCG′] |
| 1054 | NM_006892.3(DNMT3B): | 1789 | DNMT3B | [′CCAGTGGTTTGGCGATGGCAAGTTCYCCGA |
| | c.808T > C (p.Ser270Pro) | | | GGTGAGTCCGGGGAAGGGCAA′] |
| 1055 | NM_002538.3(OCLN): c.656T > C | 100506658 | OCLN | [′CAAATATATGCCCTCTGCAACCAATYTTATA |
| | (p.Phe219Ser) | | | CACCTGCAGCTACTGGACTC′] |
| 1056 | NM_001211.5(BUB1B): | −1 | — | [′AATGATGAGGCCACAGTGTCTGTTCYTGGG |
| | c.3035T > C (p.Leu1012Pro) | | | GAGCTTGCAGCAGAAATGAAT′] |
| 1057 | NM_016203.3(PRKAG2): | 51422 | PRKAG2 | [′ACAGAATCTTGCTGCTGAGAAAACAYACAA |
| | c.1459T > C (p.Tyr487His) | | | TAACCTAGATATCACGGTGAC′] |
| 1058 | NM_016203.3(PRKAG2): | 51422 | PRKAG2 | [′TAGTATTGTGGGTATTATTTCCCTGYCGGAC |
| | c.1642T > C (p.Ser548Pro) | | | ATTCTGCAAGCCCTGATCCT′] |
| 1059 | NM_001287.5(CLCN7): | 1186 | CLCN7 | [′TTCAAGCTGTTCCGGGCCCTGGGCCYGCGGC |
| | c.2297T > C (p.Leu766Pro) | | | ACCTGGTGGTGGTGGACAAC′] |
| 1060 | NM_004646.3(NPHS1): c.793T > | 4868 | NPHS1 | [′GGCAGGACAGAGCTTGGAGCTGCCGYGCGT |
| | C (p.Cys265Arg) | | | GGCCCGAGGGGGTAATCCCTT′] |
| 1061 | NM_003002.3(SDHD): c.416T > C | 6392 | SDHD | [′CTTTCAGCTTTAACCTTTGCTGGGCYTTGCT |
| | (p.Leu139Pro) | | | ATTTCAACTATCACGATGTG′] |
| 1062 | NM_001164277.1(SLC37A4): | 2542 | SLC37A4 | [′TAATGGCCTGGCCCAGGGGCTGGGCYGGCC |
| | c.352T > C (p.Trp118Arg) | | | CCCATGTGGGAAGGTCCTGCG′] |
| 1063 | NM_002546.3(TNFRSF11B): | 4982 | TNFRSF11B | [′GGAAGGGCGCTACCTTGAGATAGAGYTCTG |
| | c.349T > C (p.Phe117Leu) | | | CTTGAAACATAGGAGCTGCCC′] |
| 1064 | NM_005422.2(TECTA): | 7007 | TECTA | [′GGGCGTGAGGATCAATGACAGACAGBGCAC |
| | c.5509T> C (p.Cys1837Arg) | | | CGGCATCGAGGGGGAAGATTT′] |
| 1065 | NM_007262.4(PARK7): c.497T > | 11315 | PARK7 | [′CCTGGGACCAGCTTCGAGTTTGCGCYTGCAA |
| | C (p.Leu166Pro) | | | TTGTTGAAGCCCTGAATGGC′] |
| 1066 | NM_006009.3(TUBA1A): | 7846 | TUBA1A | [′GCTCGCCTGGACCACAAGTTTGACCYGATGT |
| | c.1190T > C (p.Leu397Pro) | | | ATGCCAAACGTGCCTTTGTT′] |
| 1067 | NM_002700.2(POU4F3): | 5459 | POU4F3 | [′CTCAAGATCCCCGGCGTGGGCTCGCYGAGC |
| | c.668T > C (p.Leu223Pro) | | | CAAAGCACCATCTGCAGGTTC′] |
| 1068 | NM_005025.4(SERPINI1): | 5274 | SERPINI1 | [′TGGTGAAGATGAAAATATTCTCTTCYCTCCA |
| | c.145T > C (p.Ser49Pro) | | | TTGAGTATTGCTCTTGCAAT′] |
| 1069 | NM_001040667.2(HSF4): | 3299 | HSF4 | [′TTCGTGCGCGGCCGCGAGCAGCTACYGGAG |
| | c.341T > C (p.Leu114Pro) | | | CGCGTGCGGCGCAAGGTGGGG′] |
| 1070 | NM_002942.4(ROBO2): | 6092 | ROBO2 | [′AATAGCAACAGTGGCCCAAATGAGAYTGGA |
| | c.2834T > C (p.Ile945Thr) | | | AATTTTGGCCGTGGAGGTAAG′] |
| 1071 | NM_000492.3(CFTR): c.3857T > | 1080 | CFTR | [′ACTTTGCAACAGTGGAGGAAAGCCTYTGGA |
| | C (p.Phe1286Ser) | | | GTGATACCACAGGTGAGCAAA′] |
| 1072 | NM_000492.3(CFTR): c.3763T > | 1080 | CFTR | [′TGGATCAGGGAAGAGTACTTTGTTAYCAGCT |
| | C (p.Ser1255Pro) | | | TTTTTGAGACTACTGAACAC′] |
| 1073 | NM_000492.3(CFTR): c.3194T > | 1080 | CFTR | [′ACAAGCTTAAAAGGACTATGGACACBTCGT |
| | C (p.Leu1065Pro) | | | GCCTTCGGACGGCAGCCTTAC′] |
| 1074 | NM_000492.3(CFTR): | 1080 | CFTR | [′TGTCTGCCATTCTTAAAAACAAAAAYGTTGT |
| | c.3469 − 20T > C | | | TATTTTTATTTCAGATGCGA′] |
| 1075 | NM_005603.4(ATP8B1): | 5205 | ATP8B1 | [′TTTTGGAGAAACACAAGTTTTCCTTYGGATG |
| | c.863T > C (p.Leu288Ser) | | | CTGATAAAATTTTGTTACGT′] |
| 1076 | NM_005603.4(ATP8B1): | 5205 | ATP8B1 | [′GAGGAGATTGAAAAAGACTTAATTGYGAGT |
| | c.2097 + 2T > C | | | TTTAGCCTTAATAACTTTTTC′] |
| 1077 | NM_005603.4(ATP8B1): | 5205 | ATP8B1 | [′ACCCTATGCCTTTGCTACAAGGAAAYTGAA |
| | c.1982T > C (p.Ile661Thr) | | | GAAAAAGAATTTACAGAATGG′] |
| 1078 | NM_005144.4(HR): | 55806 | HR | [′CGCAGCACGGAGTCTCGGCGTCCCAYGGCG |
| | c.−320T > C | | | CAACCTACGGCCTCGGCCCAG′] |
| 1079 | NM_003322.4(TULP1): | 7287 | TULP1 | [′GGTCACCCAGGCCTCAGTCAAGAACYTCCA |
| | c.1471T > C (p.Phe491Leu) | | | GATTGTCCACGCTGATGACCG′] |
| 1080 | NM_003322.4(TULP1): | 7287 | TULP1 | [′CTCCTGGGGAACCGCTTCACGGTCTYTGACA |
| | c.1145T > C (p.Phe382Ser) | | | ACGGGCAGAACCCACAGCGT′] |
| 1081 | NM_000455.4(STK11): c.200T > | 6794 | STK11 | [′TCTTACGGCAAGGTGAAGGAGGTGCYGGAC |
| | C (p.Leu67Pro) | | | TCGGAGACGCTGTGCAGGAGG′] |
| 1082 | NM_002241.4(KCNJ10): | 3766 | KCNJ10 | [′TGGCTTCCGCTACATCAGTGAGGAAYGTCCA |
| | c.418T > C (p.Cys140Arg) | | | CTGGCCATTGTGCTTCTTAT′] |
| 1083 | NM_001172567.1(MYD88): | 4615 | MYD88 | [′TCTGTAGGCCGACTGCTCGAGCTGCYTACCA |
| | c.317T > C (p.Leu106Pro) | | | AGCTGGGCCGCGACGACGTG′] |
| 1084 | NM_000466.2(PEX1): c.1991T > | 5189 | PEX1 | [′CCATCTGTTGTCCTGCTGGATGACCYTGACC |
| | C (p.Leu664Pro) | | | TCATTGCTGGACTGCCTGCT′] |
| 1085 | NM_001303.3(COX10): c.2T > C | 1352 | COX10 | [′GGAGCGGCCCCAGACTCGTAAATTAYGGCC |
| | (p.Met1Thr) | | | GCATCTCCGCACACTCTCTCC′] |
| 1086 | NM_001300.5(KLF6): c.346T > C | 1316 | KLF6 | [′TGTCAGCAGCGAATCCTCTGACAGCYCCGA |
| | (p.Ser116Pro) | | | GGAACTTTCTCCCACGGCCAA′] |
| 1087 | NM_001300.5(KLF6): c.190T > C | 1316 | KLF6 | [′CAAATTTGACAGCCAGGAAGATCTGYGGAC |
| | (p.Trp64Arg) | | | CAAAATCATTCTGGCTCGGGA′] |
| 1088 | NM_001300.5(KLF6): c.506T > C | 1316 | KLF6 | [′CTGTGGGGTTGCGTGCCCGGGGAGCYGCCC |
| | (p.Leu169Pro) | | | TCGCCAGGGAAGGTGCGCAGC′] |
| 1089 | NM_000085.4(CLCNKB): | 1188 | CLCNKB | [′CGGGTACTTCATGCCCATCTTTGTCYATGGT |
| | c.1294T > C (p.Tyr432His) | | | GAGTCTGGGGTCCTGAGGTT′] |
| 1090 | NM_000214.2(JAG1): c.110T > C | 182 | JAG1 | [′TGTGGGGCCTCGGGTCAGTTCGAGTYGGAG |
| | (p.Leu37Ser) | | | ATCCTGTCCATGCAGAACGTG′] |
| 1091 | NM_003865.2(HESX1): c.77T > C | 8820 | HESX1 | [′ACTTGCTCCTTTTCAATTGAGAGAAYCTTAG |
| | (p.Ile26Thr) | | | GACTGGACCAGAAGAAAGAC′] |
| 1092 | NM_003865.2(HESX1): | 8820 | HESX1 | [′TCATTATTGGGTGAAAAAACTTCCCDCCTGG |
| | c.357 + 2T > C | | | TTTTGAGTAAAAGCAGTTCT′] |
| 1093 | NM_002618.3(PEX13): c.977T > | 5194 | PEX13 | [′CTTGATGGCCAAACAACAGGACTTAYACCT |
| | C (p.Ile326Thr) | | | GCGAATTATGTCAAAATTCTT′] |
| 1094 | NM_000303.2(PMM2): c.395T > | 5373 | PMM2 | [′AATGGGATGTTAAACGTGTCCCCTAYTGGA |
| | C (p.Ile132Thr) | | | AGAAGCTGCAGCCAAGAAGAA′] |
| 1095 | NM_000303.2(PMM2): c.131T > | 5373 | PMM2 | [′AGGCAGAAGATCAAAATCGGAGTGGYAGGC |
| | C (p.Val44Ala) | | | GGATCGGACTTTGAGAAAGTG′] |
| 1096 | NM_000104.3(CYP1B1): c.2T > C | 1545 | CYP1B1 | [′CGCCTTCTCCTCTCTGTCCCCAGCAYGGGCA |
| | (p.Met1Thr) | | | CCAGCCTCAGCCCGAACGAC′] |
| 1097 | NM_032977.3(CASP10): | 843 | CASP10 | [′AAAGACTCGCTTCCCAAAACTGAAAYGGTG |
| | c.440T > C (p.Met147Thr) | | | AGTGGGTCATACAGAATGGGT′] |
| 1098 | NM_003999.2(OSMR): c.2072T > | 9180 | OSMR | [′GGTTCAGAATGTTGCAAATACAAAAYTGAC |
| | C (p.Ile691Thr) | | | AACCCGGAAGAAAAGGCATTG′] |
| 1099 | NM_000314.6(PTEN): c.370T > C | 5728 | PTEN | [′TGACAATCATGTTGCAGCAATTCACBGTAAA |
| | (p.Cys124Arg) | | | GCTGGAAAGGGACGAACTGG′] |
| 1100 | NM_000314.6(PTEN): c.209T > C | 5728 | PTEN | [′AAAAACCATTACAAGATATACAATCYGTAA |
| | (p.Leu70Pro) | | | GTATGTTTTCTTATTTGTATG′] |
| 1101 | NM_000314.6(PTEN): c.335T > C | 5728 | PTEN | [′TTTTGTGAAGATCTTGACCAATGGCYAAGTG |
| | (p.Leu112Pro) | | | AAGATGACAATCATGTTGCA′] |
| 1102 | NM_000314.6(PTEN): c.722T > C | 5728 | PTEN | [′CGACGGGAAGACAAGTTCATGTACTYTGAG |
| | (p.Phe241Ser) | | | TTCCCTCAGCCGTTACCTGTG′] |
| 1103 | NM_006949.3(STXBP2): | 6813 | STXBP2 | [′TTGGCCCACGCCGTCCTGGCCAAGCHGAAC |
| | c.626T > C (p.Leu209Pro) | | | GCCTTCAAGGCAGACACTCCC′] |
| 1104 | NM_000358.2(TGFBI): c.1619T > | 7045 | TGFBI | [′AACCGGGAAGGAGTCTACACAGTCTYTGCT |
| | C (p.Phe540Ser) | | | CCCACAAATGAAGCCTTCCGA′] |
| 1105 | NM_000350.2(ABCA4): | 24 | ABCA4 | [′CAGCTCACCCAACGTGCCCTCTCTCYACTGG |
| | c.1622T > C (p.Leu541Pro) | | | AGGAAAACATGTTCTGGGCC′] |
| 1106 | NM_000350.2(ABCA4): | 24 | ABCA4 | [′GGAAATAAAACTGACATCTTAAGGCYACAT |
| | c.5819T > C (p.Leu1940Pro) | | | GAACTAACCAAGGTAAGGGAA′] |
| 1107 | NM_000860.5(HPGD): c.577T > C | 3248 | HPGD | [′CTTTGTTAACACAGCCATCCTTGAAYCAATT |
| | (p.Ser193Pro) | | | GAAAAAGAAGAAAACATGGG′] |
| 1108 | NM_000223.3(KRT12): c.386T > | 3859 | KRT12 | [′CTTTCTGGATCAGAAAAAGAAACTAYGCAA |
| | C (p.Met129Thr) | | | AATCTTAATGATAGATTAGCT′] |
| 1109 | NM_000503.5(EYA1): c. 1459T > | 2138 | EYA1 | [′GTTGACACTGGCCCTGAAAGCACTCYCGCTC |
| | C (p.Ser487Pro) | | | ATTCACTCCCGGTGAGGCTC′] |
| 1110 | NM_000261.1(MYOC): | 4653 | MYOC | [′TGCCTTCATCATCTGTGGCACCTTGYACACC |
| | c.1309T > C (p.Tyr437His) | | | GTCAGCAGCTACACCTCAGC′] |
| 1111 | NM_000261.1(MYOC): | 4653 | MYOC | [′GTCAGTCGCCAATGCCTTCATCATCYGTGGC |
| | c.1297T > C (p.Cys433Arg) | | | ACCTTGTACACCGTCAGCAG′] |
| 1112 | NM_130838.1(UBE3A): c.389T > | 7337 | UBE3A | [′GATTATTCCCCTTTAATCCGTGTTAYTGGAA |
| | C (p.Ile130Thr) | | | GAGTTTTTTCTAGTGCTGAG′] |
| 1113 | NM_000474.3(TWIST1): | 7291 | TWIST1 | [′TCGCTGAACGAGGCGTTCGCCGCGCYGCGG |
| | c.392T > C (p.Leu131Pro) | | | AAGATCATCCCCACGCTGCCC′] |
| 1114 | NM_000192.3(TBX5): c.161T > C | 6910 | TBX5 | [′TCCTTCTTGCAGGGCATGGAGGGAAYCAAA |
| | (p.Ile54Thr) | | | GTGTTTCTCCATGAAAGAGAA′] |
| 1115 | NM_002905.3(RDH5): c.841T > C | −1 | — | [′GACTGCTCGACACCCCCGAACCCGCBACAG |
| | (p.Tyr281His) | | | CCCAGGTTGGGATGCCAAGCT′] |
| 1116 | NM_001089.2(ABCA3): c.302T > | 21 | ABCA3 | [′GTCACTGAGACAGTGCGCAGGGCACYTGTG |
| | C (p.Leu101Pro) | | | ATCAACATGCGAGGTGAGACA′] |
| 1117 | NM_001089.2(ABCA3): | 21 | ABCA3 | [′ATGGACCCCGTGGCCCGGCGCCTGCYTTGG |
| | c.4658T > C (p.Leu1553Pro) | | | GACACCGTGGCACGAGCCCGA′] |
| 1118 | NM_001089.2(ABCA3): c.977T > | 21 | ABCA3 | [′ATCGCCGCCTCCTTCATGACCCTGCYCTTCT |
| | C (p.Leu326Pro) | | | GTGTCAAGGTGAGCATGGCG′] |
| 1119 | NM_005535.2(IL12RB1): | 3594 | IL12RB1 | [′CTGCCTCTCCCCAGAGTCCTGCCTCYGCCCC |
| | c.592T > C (p.Cys198Arg) | | | CTGGAGATGAATGTGGCCCA′] |
| 1120 | NM_005055.4(RAPSN): c.41T > C | 5913 | RAPSN | [′ACCAAGCAGCAGATCGAGAAGGGGCYCCAG |
| | (p.Leu14Pro) | | | CTGTACCAGTCCAACCAGACA′] |
| 1121 | NM_005055.4(RAPSN): c.848T > | 5913 | RAPSN | [′ATCATGACCGAGATCGGAAACCGCCYGGGG |
| | C (p.Leu283Pro) | | | CAGGTGCAGGCGCTGCTGGGT′] |
| 1122 | NM_005055.4(RAPSN): c.416T > | 5913 | RAPSN | [′AATGCCTTCCTGGGCCTCAGCGTCTYCCAGA |
| | C (p.Phe139Ser) | | | AGGCCCTGGAGAGCTTCGAG′] |
| 1123 | NM_005570.3(LMAN1): c.2T > C | 3998 | LMAN1 | [′CTCCTCCGCGTTCCAGAATCCAAGAYGGCG |
| | (p.Met1Thr) | | | GGATCCAGGCAAAGGGGTCTC′] |
| 1124 | NM_000430.3(PAFAH1B1): | 5048 | PAFAH1B1 | [′CAGCGGCAAGCTTCTGGCTTCCTGTYCTGCA |
| | 5c.50T > C (p.Ser169Pro) | | | GATATGACCATTAAACTATG′] |
| 1125 | NM_000430.3(PAFAH1B1): | 5048 | PAFAH1B1 | [′GGCTATGAAGAGGCATATTCAGTTTYTAAA |
| | c.92T > C (p.Phe31Ser) | | | AAGGAAGCTGAATTAGATGTG′] |
| 1126 | NM_006261.4(PROP1): c.263T > | 5626 | PROP1 | [′CAGTTGGAACAGCTGGAGTCAGCCTYTGGG |
| | C (p.Phe88Ser) | | | AGGAACCAGTACCCCGACATC′] |
| 1127 | NM_175929.2(FGF14): c.449T > | 2259 | FGF14 | [′GAACTTTTTACCCCTGAATGCAAGTYTAAAG |
| | C (p.Phe150Ser) | | | AATCTGTTTTTGAAAATTAT′] |
| 1128 | NM_004970.2(IGFALS): | 3483 | IGFALS | [′GTGGCTGGAGGGTAACCCCTGGGACYGTGG |
| | c.1618T > C (p.Cys540Arg) | | | CTGCCCTCTCAAGGCGCTGCG′] |
| 1129 | NM_005379.3(MYO1A): | 4640 | MYO1A | [′CCCTTGCCTGTTCCGTCCCTAGACTYCTTCTC |
| | c.2728T > C (p.Ser910Pro) | | | GGATTCTCCTCCTGACCAA′] |
| 1130 | NM_006302.2(MOGS): c.1954T > | 7841 | MOGS | [′GCACTGGGCCCCAGAGCTAGGAGTCYTTGC |
| | C (p.Phe652Leu) | | | AGACTTTGGGAACCACACAAA′] |
| 1131 | NM_001009944.2(PKD1): | 5310 | PKD1 | [′TACGTGCCCACCAACGGCTCAGCCTYGGTGC |
| | c.2534T > C (p.Leu845Ser) | | | TCCAGGTGGACTCTGGTGCC′] |
| 1132 | NM_004329.2(BMPR1A): | 657 | BMPR1A | [′AGCCCAGCTACGCCGGACAATAGAAYGTTG |
| | c.370T > C (p.Cys124Arg) | | | TCGGACCAATTTATGTAACCA′] |
| 1133 | NM_004329.2(BMPR1A): | 657 | BMPR1A | [′CCGAGTGATCCGTCATACGAAGATAYGCGT |
| | c.1409T > C (p.Met470Thr) | | | GAGGTTGTGTGTGTCAAACGT′] |
| 1134 | NM_000452.2(SLC10A2): | 6555 | SLC10A2 | [′GTGGCGGGTTACTCCCTGGGGTTTCYTCTGG |
| | c.728T > C (p.Leu243Pro) | | | CTAGAATTGCTGGTCTACCC′] |
| 1135 | NM_030662.3(MAP2K2): | 5605 | MAP2K2 | [′CAACTCGCCGTACATCGTGGGCTTCYACGGG |
| | c.400T > C (p.Tyr134His) | | | GCCTTCTACAGTGACGGGGA′] |
| 1136 | NM_033337.2(CAV3): c.260T > C | 859 | CAV3 | [′CTGCTGGGCGTCCCACTGGCCCTGCHCTGGG |
| | (p.Leu87Pro) | | | GCTTCCTGTTCGCCTGCATC′] |
| 1137 | NM_000388.3(CASR): c.2417T > | 846 | CASR | [′CCGGAGAACTTCAATGAAGCCAAGTHCATC |
| | C (p.Phe806Ser) | | | ACCTTCAGCATGCTCATCTTC′] |
| 1138 | NM_000388.3(CASR): c.382T > C | 846 | CASR | [′AATTGATTCTTTGAACCTTGATGAGYTCTGC |
| | (p.Phe128Leu) | | | AACTGCTCAGAGCACATTCC′] |
| 1139 | NM_000388.3(CASR): c.1835T > | 846 | CASR | [′GAGTTTCTGTCGTGGACGGAGCCCTYTGGGA |
| | C (p.Phe612Ser) | | | TCGCACTCACCCTCTTTGCC′] |
| 1140 | NM_000388.3(CASR): c.2641T > | 846 | CASR | [′GCGTTGCAGCACCGCAGCTCACGCTYTCAA |
| | C (p.Phe881Leu) | | | GGTGGCTGCCCGGGCCACGCT′] |
| 1141 | NM_000388.3(CASR): c.374T > C | 846 | CASR | [′CAAAACAAAATTGATTCTTTGAACCYTGATG |
| | (p.Leul25Pro) | | | AGTTCTGCAACTGCTCAGAG′] |
| 1142 | NM_000388.3(CASR): c.2362T > | 846 | CASR | [′CACCTGCCTGCTGGCTGCCATCTGCYTCTTC |
| | C (p.Phe788Leu) | | | TTTGCCTTCAAGTCCCGGAA′] |
| 1143 | NM_000388.3(CASR): c.38T > C | 846 | CASR | [′AGCTGCTGCTGGGTCCTCTTGGCACYCACCT |
| | (p.Leu13Pro) | | | GGCACACCTCTGCCTACGGG′] |
| 1144 | NM_030653.3(DDX11): | 1663 | DDX11 | [′CTGGCATATTCCAGGTGCATCCAGGYGCGG |
| | c.2271 + 2T > C | | | GCGTCATGCTGGGCTTGGGTC′] |
| 1145 | NM_001001557.2(GDF6): | 392255 | GDF6 | [′TTCACCAGATCCCAGCGCAAGAACCYGTTC |
| | c.866T > C (p.Leu289Pro) | | | GCAGAGATGCGCGAGCAGCTG′] |
| 1146 | NM_000557.4(GDF5): c.1322T > | −1 | — | [′TGCGAGTTCCCATTGCGCTCCCACCYGGAGC |
| | C (p.Leu441Pro) | | | CCACGAATCATGCAGTCATC′] |
| 1147 | NM_000392.4(ABCC2): | 1244 | ABCC2 | [′TGATTCGGAAGCCACAGTCCGAGAGYGAGT |
| | c.1967 + 2T > C | | | TGCCTTCTTTCCATCCTAATG′] |
| 1148 | NM_000396.3(CTSK): c.926T > C | 1513 | CTSK | [′AACTGGGGAAACAAAGGATATATCCYCATG |
| | (p.Leu309Pro) | | | GCTCGAAATAAGAACAACGCC′] |
| 1149 | NM_000304.3(PMP22): c.47T > C | 5376 | PMP22 | [′ATCATCGTCCTCCACGTCGCGGTGCYGGTGC |
| | (p.Leul6Pro) | | | TGCTGTTCGTCTCCACGATC′] |
| 1150 | NM_000304.3(PMP22): c.82T > C | 5376 | PMP22 | [′ATATCTATCTGATTCTCTCTAGCAAYGGATC |
| | (p.Trp28Arg) | | | GTGGGCAATGGACACGCAAC′] |
| 1151 | NM_001453.2(FOXC1): c.335T > | 2296 | FOXC1 | [′ATCTACCAGTTCATCATGGACCGCTHCCCCT |
| | C (p.Phe112Ser) | | | TCTACCGGGACAACAAGCAG′] |
| 1152 | NM_001451.2(FOXF1): | 2294 | FOXF1 | [′CCAAGACATCAAGCCTTGCGTGATGYGAGG |
| | c.1138T > C (p.Ter380Arg) | | | CTGCCGCCGCAGGCCCTCCTG′] |
| 1153 | NM_006432.3(NPC2): c.199T > C | 10577 | NPC2 | [′CTCTTTTTTTCTCTTAGATATTCAGYCTAAAA |
| | (p.Ser67Pro) | | | GCAGCAAGGCCGTGGTGCA′] |
| 1154 | NM_001127221.1(CACNA1A): | 773 | CACNA1A | [′CTGAATGTGTTCTTGGCCATCGCTGYGGACA |
| | c.2141T > C (p.Val714Ala) | | | ATCTGGCCAACGCCCAGGAG′] |
| 1155 | NM_001127221.1(CACNA1A): | 773 | CACNA1A | [′GGGTACCGCATGGAGATGTCCATTTYCTACG |
| | c.4469T > C (p.Phe1490Ser) | | | TCGTCTACTTTGTGGTGTTC′] |
| 1156 | NM_001127221.1(CACNA1A): | 773 | CACNA1A | [′ATGCTCTTCTTCATCTATGCCATCAYTGGGA |
| | c.5126T > C (p.Ile1709Thr) | | | TGCAGGTGAGTGTCGTGTCC′] |
| 1157 | NM_000901.4(NR3C2): | 4306 | NR3C2 | [′CAGAGCTGGCAGAGGTTCTACCAACYGACC |
| | c.2771T > C (p.Leu924Pro) | | | AAGCTGCTGGACTCCATGCAT′] |
| 1158 | NM_000901.4(NR3C2): | 4306 | NR3C2 | [′GTGGAGTCGGGGAACGCCAAGCCGCYCTAC |
| | c.2936T > C (p.Leu979Pro) | | | TTCCACCGGAAGTGACTGCCC′] |
| 1159 | NM_000339.2(SLC12A3): | 6559 | SLC12A3 | [′CTCACCCTCCTCATTCCCTATCTCCYTGGCC |
| | c.2576T > C (p.Leu859Pro) | | | GCAAGAGGAGGTGGAGCAAA′] |
| 1160 | NM_000339.2(SLC12A3): | 6559 | SLC12A3 | [′CTGGGGTGCCTGCGAGGGGCTGGCCYGCAG |
| | c.1261T > C (p.Cys421Arg) | | | CTATGGCTGGAACTTCACCGA′] |
| 1161 | NM_000339.2(SLC12A3): | 6559 | SLC12A3 | [′TCGGTACAGGCTGGCTCCTACAACCYGGCCC |
| | c.1868T > C (p.Leu623Pro) | | | TCAGCTACTCGGTGGGCCTC′] |
| 1162 | NM_000163.4(GHR): c.341T > C | 2690 | GHR | [′TCTGCTGGGGAAAACAGCTGTTACTYTAATT |
| | (p.Phe114Ser) | | | CATCGTTTACCTCCATCTGG′] |
| 1163 | NM_000163.4(GHR): c.512T > C | 2690 | GHR | [′AGTTTAACTGGGATTCATGCAGATAYCCAA |
| | (p.Ile171Thr) | | | GTGAGATGGGAAGCACCACGC′] |
| 1164 | NM_000525.3(KCNJ11): | 3767 | KCNJ11 | [′ACTGAGGAGTGCCCACTGGCCATCCYGATC |
| | c.440T > C (p.Leu147Pro) | | | CTCATCGTGCAGAACATCGTG′] |
| 1165 | NM_000525.3(KCNJ11): | 3767 | KCNJ11 | [′CCGCTTTGTGTCCAAGAAAGGCAACYGCAA |
| | c.124T > C (p.Cys42Arg) | | | CGTGGCCCACAAGAACATCCG′] |
| 1166 | NM_001122764.1(PPOX): | 5498 | PPOX | [′ACCGTGGTCGTGCTGGGCGGAGGCAYCAGC |
| | c.35T > C (p.Ile12Thr) | | | GGCTTGGCCGCCAGTTACCAC′] |
| 1167 | NM_000182.4(HADHA): | 3030 | HADHA | [′AAAGAATCAAAGGCCTTGATGGGACYCTAC |
| | c.1025T > C (p.Leu342Pro) | | | CATGGTCAGGTCCTGTGCAAG′] |
| 1168 | NM_003476.4(CSRP3): c.131T > | 8048 | CSRP3 | [′CTGGCAGTGGCCTGCAGGAAGGCTCYTGAC |
| | C (p.Leu44Pro) | | | AGCACGACAGTCGCGGCTCAT′] |
| 1169 | NM_001204.6(BMPR2): c.367T > | 659 | BMPR2 | [′TTTCTGCTGTTGTAGCACAGATTTAHGTAAT |
| | C (p.Cys123Arg) | | | GTCAACTTTACTGAGAATTT′] |
| 1170 | NM_000336.2(SCNN1B): | 6338 | SCNN1B | [′CATCCCAGGCACCCCGCCCCCCAACYATGA |
| | c.1858T > C (p.Tyr620His) | | | CTCCCTGCGTCTGCAGCCGCT′] |
| 1171 | NM_002181.3(IHH): c.569T > C | 3549 | IHH | [′TCAAAGGCCCACGTGCATTGCTCCGYCAAGT |
| | (p.Val190Ala) | | | CCGGTGAGCCGCCGCCGGGG′] |
| 1172 | NM_000193.3(SHH): c.349T > C | 6469 | SHH | [′TTTGGCCATCTCGGTGATGAACCAGBGGCCA |
| | (p.Trp117Arg) | | | GGAGTGAAACTGCGGGTGAC′] |
| 1173 | NM_000193.3(SHH): c.995T > C | 6469 | SHH | [′GACCGCCGGCTCCTGCCCGCCGCTGYGCAC |
| | (p.Val332Ala) | | | AGCGTGACCCTAAGCGAGGAG′] |
| 1174 | NM_000179.2(MSH6): c.2633T > | 2956 | MSH6 | [′AAAATTATAGGGATCATGGAAGAAGBTGCT |
| | C (p.Val878Ala) | | | GATGGTTTTAAGTCTAAAATC′] |
| 1175 | NM_000098.2(CPT2): c.1342T > | 1376 | CPT2 | [′AACCCTCACTATTGACTGCGTCCAGYTTCAG |
| | C (p.Phe448Leu) | | | AGAGGAGGCAAAGAATTCCT′] |
| 1176 | NM_007315.3(STAT1): | 6772 | STAT1 | [′GGAACTGGATATATCAAGACTGAGTYGATT |
| | c.2117T > C (p.Leu706Ser) | | | TCTGTGTCTGAAGTGTAAGTG′] |
| 1177 | NM_007315.3(STAT1): | 6772 | STAT1 | [′AAGGACCAGCAGCCGGGGACCTTCCYGCTG |
| | c.1799T > C (p.Leu600Pro) | | | CGGTTCAGTGAGAGCTCCCGG′] |
| 1178 | NM_024420.2(PLA2G4A): | 5321 | PLA2G4A | [′AGGGACAGCAACATTTACTGTATCTYCTATG |
| | c.331T > C (p.Ser111Pro) | | | AAGGTGGGAGAAAAGAAAGA′] |
| 1179 | NM_000352.4(ABCC8): c.394T > | 6833 | ABCC8 | [′CTATCACAACATCGAGACTTCCAACBTCCCC |
| | C (p.Phe132Leu) | | | AAGCTGCTAATTGGTAGGTG′] |
| 1180 | NM_033163.3(FGF8): c.118T > C | 2253 | FGF8 | [′GCTGGGCAGGGAGCTCGCTTCCCTGYTCCGG |
| | (p.Phe40Leu) | | | GCTGGCCGGGAGCCCCAGGG′] |
| 1181 | NM_153767.3(KCNJ1): | 3758 | KCNJ1 | [′AACATCTTTCTCATTATAAAGGCACRTGGCA |
| | c.1013T > C (p.Met338Thr) | | | CAGTGAGGGGTCTCCACTTC′] |
| 1182 | NM_000095.2(COMP): | 1311 | COMP | [′AACCCACCCTGTCATCCAGGACAACYGCCC |
| | c.982T > C (p.Cys328Arg) | | | GCTGGTGCGGAACCCAGACCA′] |
| 1183 | NM_000095.2(COMP): c.1042T > | 1311 | COMP | [′GGACGAGGACAAGTGGGGCGATGCGYGCGA |
| | C (p.Cys348Arg) | | | CAACTGCCGGTCCCAGAAGAA′] |
| 1184 | NM_002047.2(GARS): c.548T > C | 2617 | GARS | [′ATCCTGGAGATCGATTGCACCATGCYCACCC |
| | (p.Leu183Pro) | | | CTGAGCCAGTTTTAAAGTGA′] |
| 1185 | NM_000435.2(NOTCH3): | 4854 | NOTCH3 | [′CCTCGACCGCATAGGCCAGTTCACCYGTATC |
| | c.1363T > C (p.Cys455Arg) | | | TGTATGGCAGGTGGGTGGTG′] |
| 1186 | NM_001166107.1(HMGCS2): | 3158 | HMGCS2 | [′CTGCTACGGTGGTACTGCCTCCCTCYTCAAT |
| | c.520T > C (p.Phe174Leu) | | | GCTGCCAACTGGATGGAGTC′] |
| 1187 | NM_001038.5(SCNN1A): | 6337 | SCNN1A | [′CCAGCTCTCTGCTGGTTACTCACGAYGGCCC |
| | c.1477T > C (p.Trp493Arg) | | | TCGGTGACATCCCAGGTAGA′] |
| 1188 | NM_000161.2(GCH1): c.662T > C | 2643 | GCH1 | [′ATGGTAATGCGAGGTGTACAGAAAAYGAAC |
| | (p.Met221Thr) | | | AGCAAAACTGTGACCAGCACA′] |
| 1189 | NM_000059.3(BRCA2): | 675 | BRCA2 | [′CGCGTCTTTCCACAGCCAGGCAGTCYGTATC |
| | c.7529T > C (p.Leu2510Pro) | | | TTGCAAAAACATCCACTCTG′] |
| 1190 | NM_000180.3(GUCY2D): | 3000 | GUCY2D | [′GGAGACAGGGTTTGGCTGAAGAAATYCCCA |
| | c.1694T > C (p.Phe565Ser) | | | GGGGATCAGCACATAGCTATC′] |
| 1191 | NM_000180.3(GUCY2D): | 3000 | GUCY2D | [′AATGGGCAGCGACACGCGGCAGAGAYCGCC |
| | c.2846T > C (p.Ile949Thr) | | | AACATGTCACTGGACATCCTC′] |
| 1192 | NM_198056.2(SCN5A): | 6331 | SCN5A | [′CTGTCACGATTTGAGGGCATGAGGGYAAGA |
| | c.3963 + 2T > C | | | GAGGTGGCTGCCTTCCCACCA′] |
| 1193 | NM_000335.4(SCN5A): | 6331 | SCN5A | [′GAGTGAGGACGACTTCGATATGTTCYATGA |
| | c.5380T > C (p.Tyr1794His) | | | GATCTGGGAGAAATTTGACCC′] |
| 1194 | NM_000023.2(SGCA): c.524T > C | 6442 | SGCA | [′CCCGGAGAGCTTCAGCTGCTCAACGYCACCT |
| | (p.Val175Ala) | | | CTGCCTTGGACCGTGGGGGC′] |
| 1195 | NM_002427.3(MMP13): c.224T > | 4322 | MMP13 | [′AGGCTCCGAGAAATGCAGTCTTTCTYCGGCT |
| | C (p.Phe75Ser) | | | TAGAGGTGACTGGCAAACTT′] |
| 1196 | NM_002427.3(MMP13): c.221T > | 4322 | MMP13 | [′GAGAGGCTCCGAGAAATGCAGTCTTYCTTC |
| | C (p.Phe74Ser) | | | GGCTTAGAGGTGACTGGCAAA′] |
| 1197 | NM_002427.3(MMP13): c.272T > | 4322 | MMP13 | [′CTTGACGATAACACCTTAGATGTCAYGAAA |
| | C (p.Met91Thr) | | | AAGCCAAGATGCGGGGTTCCT′] |
| 1198 | NM_004525.2(LRP2): c.7564T > | 4036 | LRP2 | [′CTCCTTTTGTATGACTAGGTACCTGYACTGG |
| | C (p.Tyr2522His) | | | GCTGACTGGGATACACATGC′] |
| 1199 | NM_000211.4(ITGB2): c.446T > | 3689 | ITGB2 | [′GATGACCTCAGGAATGTCAAGAAGCYAGGT |
| | C (p.Leu149Pro) | | | GGCGACCTGCTCCGGGCCCTC′] |
| 1200 | NM_000211.4(ITGB2): c.412T > | 3689 | ITGB2 | [′GTACTATCTGATGGACCTCTCCTACYCCATG |
| | C (p.Ser138Pro) | | | CTTGATGACCTCAGGAATGT′] |
| 1201 | NM_005502.3(ABCA1): | 19 | ABCA1 | [′CAAAATCAAGAAGATGCTGCCTGTGYGTCC |
| | c.4429T > C (p.Cys1477Arg) | | | CCCAGGGGCAGGGGGGCTGCC′] |
| 1202 | NM_005502.3(ABCA1): | 19 | ABCA1 | [′TTGACTGGGAGAGAACACGTGGAGTYCTTT |
| | c.6026T > C (p.Phe2009Ser) | | | GCCCTTTTGAGAGGAGTCCCA′] |
| 1203 | NM_130439.3(MXI1): | 4601 | MXI1 | [′AACAAAGCCAAAGCACACATCAAGGYGAGA |
| | c.552 + 2T> C | | | ATTTTTACTTTCAGATTTGCA′] |
| 1204 | m.5874T > C | 4579 | MT-TY | [′GTCTTTAGATTTACAGTCCAATGCTYCACTC |
| | | | | AGCCATTTTACCTCACCCCC′] |
| 1205 | m.7512T > C | 4574 | MT-TS1 | [′CCAACCCCATGGCCTCCATGACTTTYTCAAA |
| | | | | AAGGTATTAGAAAAACCATT′] |
| 1206 | m.7510T > C | 4574 | MT-TS1 | [′AGCCAACCCCATGGCCTCCATGACTYTTTCA |
| | | | | AAAAGGTATTAGAAAAACCA′] |
| 1207 | m.7511T > C | 4574 | MT-TS1 | [′GCCAACCCCATGGCCTCCATGACTTYTTCAA |
| | | | | AAAGGTATTAGAAAAACCAT′] |
| 1208 | m.616T > C | 4558 | MT-TF | [′CCTCCTCAAAGCAATACACTGAAAABGTTTA |
| | | | | GACGGGCTCACATCACCCCA′] |
| 1209 | m.4409T > C | 4569 | MT-TM | [′TATCACACCCCATCCTAAAGTAAGGYCAGCT |
| | | | | AAATAAGCTATCGGGCCCAT′] |
| 1210 | m.8356T > C | 4566 | MT-TK | [′AAAGATTAAGAGAACCAACACCTCTYTACA |
| | | | | GTGAAATGCCCCAACTAAATA′] |
| 1211 | m.12297T > C | 4568 | MT-TL2 | [′AAAGGATAACAGCTATCCATTGGTCYTAGG |
| | | | | CCCCAAAAATTTTGGTGCAAC′] |
| 1212 | m.3271T > C | 4567 | MT-TL1 | [′CCGGTAATCGCATAAAACTTAAAACYTTAC |
| | | | | AGTCAGAGGTTCAATTCCTCT′] |
| 1213 | m.3250T > C | 4567 | MT-TL1 | [′GGTTTGTTAAGATGGCAGAGCCCGGYAATC |
| | | | | GCATAAAACTTAAAACTTTAC′] |
| 1214 | m.3290T > C | 4567 | MT-TL1 | [′TAAAACTTTACAGTCAGAGGTTCAAYTCCTC |
| | | | | TTCTTAACAACATACCCATG′] |
| 1215 | m.4290T > C | 4565 | MT-TI | [′AAATATGTCTGATAAAAGAGTTACTYTGATA |
| | | | | GAGTAAATAATAGGAGCTTA′] |
| 1216 | m.4291T > C | 4565 | MT-TI | [′AATATGTCTGATAAAAGAGTTACTTYGATAG |
| | | | | AGTAAATAATAGGAGCTTAA′] |
| 1217 | m.9997T > C | 4563 | MT-TG | [′ATCTATTGATGAGGGTCTTACTCTTYTAGTA |
| | | | | TAAATAGTACCGTTAACTTC′] |
| 1218 | m.10010T > C | 4563 | MT-TG | [′GGTCTTACTCTTTTAGTATAAATAGYACCGT |
| | | | | TAACTTCCAATTAACTAGTT′] |
| 1219 | m.4336T > C | 4572 | MT-TQ | [′GCTTAAACCCCCTTATTTCTAGGACYATGAG |
| | | | | AATCGAACCCATCCCTGAGA′] |
| 1220 | m.14709T > C | 4556 | MT-TE | [′CGGACTACAACCACGACCAATGATAYGAAA |
| | | | | AACCATCGTTGTATTTCAACT′] |
| 1221 | m.14674T > C | 4556 | MT-TE | [′TCAACAGAAACAAAGCATACATCATBATTCT |
| | | | | CGCACGGACTACAACCACGA′] |
| 1222 | m.5692T > C | 4570 | MT-TN | [′GGGACTTAAACCCACAAACACTTAGYTAAC |
| | | | | AGCTAAGCACCCTAATCAACT′] |
| 1223 | m.5728T > C | 4570 | MT-TN | [′GCACCCTAATCAACTGGCTTCAATCYACTTC |
| | | | | TCCCGCCGCCGGGAAAAAAG′] |
| 1224 | m.2991T > C | 4550 | MT-RNR2 | [′TCAACAATAGGGTTTACGACCTCGAYGTTGG |
| | | | | ATCAGGACATCCCGATGGTG′] |
| 1225 | m.1291T > C | 4549 | MT-RNR1 | [′ATCTTCAGCAAACCCTGATGAAGGCYACAA |
| | | | | AGTAAGCGCAAGTACCCACGT′] |
| 1226 | m.1095T > C | 4549 | MT-RNR1 | [′CTGGGATTAGATACCCCACTATGCTYAGCCC |
| | | | | TAAACCTCAACAGTTAAATC′] |
| 1227 | m.8528T > C | −1 | — | [′TTATAACAAACCCTGAGAACCAAAAYGAAC |
| | | | | GAAAATCTGTTCGCTTCATTC′] |
| 1228 | m.8993T > C | 4508 | MT-ATP6 | [′AGCCTACTCATTCAACCAATAGCCCBGGCCG |
| | | | | TACGCCTAACCGCTAACATT′] |
| 1229 | m.9101T > C | 4508 | MT-ATP6 | [′ATTAACCTTCCCTCTACACTTATCAYCTTCA |
| | | | | CAATTCTAATTCTACTGACT′] |
| 1230 | m.9176T > C | 4508 | MT-ATP6 | [′ATCCAAGCCTACGTTTTCACACTTCBAGTAA |
| | | | | GCCTCTACCTGCACGACAAC′] |
| 1231 | m.8851T > C | 4508 | MT-ATP6 | [′AAACCTAGCCATGGCCATCCCCTTAYGAGC |
| | | | | GGGCACAGTGATTATAGGCTT′] |
| 1232 | m.9185T > C | 4508 | MT-ATP6 | [′TACGTTTTCACACTTCTAGTAAGCCYCTACC |
| | | | | TGCACGACAACACATAATGA′] |
| 1233 | m.7587T > C | 4513 | MT-CO2 | [′ATAGGCTAAATCCTATATATCTTAAYGGCAC |
| | | | | ATGCAGCGCAAGTAGGTCTA′] |
| 1234 | m.6742T > C | 4512 | MT-CO1 | [′GGTATGGTCTGAGCTATGATATCAAYTGGCT |
| | | | | TCCTAGGGTTTATCGTGTGA′] |
| 1235 | m.6721T > C | 4512 | MT-CO1 | [′AAAGAACCATTTGGATACATAGGTAYGGTC |
| | | | | TGAGCTATGATATCAATTGGC′] |
| 1236 | m.7275T > C | 4512 | MT-CO1 | [′CATGAAACATCCTATCATCTGTAGGYTCATT |
| | | | | CATTTCTCTAACAGCAGTAA′] |
| 1237 | m.15572T > C | 4519 | MT-CYB | [′CATCAAGCCCGAATGATATTTCCTAYTCGCC |
| | | | | TACACAATTCTCCGATCCGT′] |
| 1238 | m.15197T > C | 4519 | MT-CYB | [′GGCCACAGTAATTACAAACTTACTAYCCGCC |
| | | | | ATCCCATACATTGGGACAGA′] |
| 1239 | m. 14849T > C | 4519 | MT-CYB | [′CATCTCCGCATGATGAAACTTCGGCYCACTC |
| | | | | CTTGGCGCCTGCCTGATCCT′] |
| 1240 | NC_012920.1: m.14484T > C | 4541 | MT-ND6 | [′GCTGTAGTATATCCAAAGACAACCAYCATTC |
| | | | | CCCCTAAATAAATTAAAAAA′] |
| 1241 | m. 14487T > C | 4541 | MT-ND6 | [′GTAGTATATCCAAAGACAACCATCAYTCCCC |
| | | | | CTAAATAAATTAAAAAAACT′] |
| 1242 | m.12706T > C | 4540 | MT-ND5 | [′ATCAGTTCTTCAAATATCTACTCATYTTCCT |
| | | | | AATTACCATACTAATCTTAG′] |
| 1243 | m.10563T > C | 4539 | MT-ND4L | [′CTCACACCTCATATCCTCCCTACTAYGCCTA |
| | | | | GAAGGAATAATACTATCGCT′] |
| 1244 | m.10663T > C | 4539 | MT-ND4L | [′ATATTGTGCCTATTGCCATACTAGTYTTTGC |
| | | | | CGCCTGCGAAGCAGCGGTNG′] |
| 1245 | m.10191T > C | 4537 | MT-ND3 | [′TTACGAGTGCGGCTTCGACCCTATAYCCCCC |
| | | | | GCCCGCGTCCCTTTCTCCAT′] |
| 1246 | m.10158T > C | 4537 | MT-ND3 | [′ACAACTCAACGGCTACATAGAAAAAYCCAC |
| | | | | CCCTTACGAGTGCGGCTTCGA′] |
| 1247 | m.4681T > C | 4536 | MT-ND2 | [′CAAGCAACCGCATCCATAATCCTTCYAATAG |
| | | | | CTATCCTCTTCAACAATATA′] |
| 1248 | m.4160T > C | 4535 | MT-ND1 | [′TACCCCCGATTCCGCTACGACCAACYCATAC |
| | | | | ACCTCCTATGAAAAAACTTC′] |
| 1249 | m.3394T > C | 4535 | MT-ND1 | [′GCTTACCGAACGAAAAATTCTAGGCYATAT |
| | | | | ACAACTACGCAAAGGCCCCAA′] |
| 1250 | m.3308T > C | 4535 | MT-ND1 | [′GGTTCAATTCCTCTTCTTAACAACABACCCA |
| | | | | TGGCCAACCTCCTACTCCTC′] |
| 1251 | m.3949T > C | 4535 | MT-ND1 | [′ACTAGTCTCAGGCTTCAACATCGAAYACGCC |
| | | | | GCAGGCCCCTTCGCCCTATT′] |
| 1252 | NM_003140.2(SRY): c.326T > C | 6736 | SRY | [′CTTACTGAAGCCGAAAAATGGCCATYCTTCC |
| | (p.Phe109Ser) | | | AGGAGGCACAGAAATTACAG′] |
| 1253 | NM_003140.2(SRY): c.203T > C | 6736 | SRY | [′GTGAAGCGACCCATGAACGCATTCAYCGTG |
| | (p.Ile68Thr) | | | TGGTCTCGCGATCAGAGGCGC′] |
| 1254 | NM_021083.2(XK): c.880T > C | 7504 | XK | [′ACTCTATACTGGTATCAACATGTTCNGCTGG |
| | (p.Cys294Arg) | | | TCTGCTGTACAGCTGAAAAT′] |
| 1255 | NM_000354.5(SERPINA7): | 6906 | SERPINA7 | [′CACCAGATGGAACAATACTATCACCYAGTG |
| | c.740T > C (p.Leu247Pro) | | | GATATGGAATTGAACTGCACA′] |
| 1256 | NM_000044.3(AR): c.2033T > C | 367 | AR | [′TGTCAGCCCATCTTTCTGAATGTCCYGGAAG |
| | (p.Leu678Pro) | | | CCATTGAGCCAGGTGTAGTG′] |
| 1257 | NM_000044.3(AR): c.2423T > C | 367 | AR | [′ATCACCCCCCAGGAATTCCTGTGCAYGAAA |
| | (p.Met808Thr) | | | GCACTGCTACTCTTCAGCATT′] |
| 1258 | NM_000044.3(AR): c.2596T > C | 367 | AR | [′CTACCAGCTCACCAAGCTCCTGGACYCCGTG |
| | (p.Ser866Pro) | | | CAGCCTGTAAGCAAACGATG′] |
| 1259 | NM_000451.3(SHOX): c.877T > C | 6473 | SHOX | [′GAAGCACGCGGAGGCCCTGGGGCTCYGACC |
| | (p.Ter293Arg) | | | CGCCGCGCAGCCCCCCGCGCG′] |
| 1260 | NM_000330.3(RS1): c.286T > C | −1 | — | [′TTGAGCCGGGCCTTGTTTGCAGTCCRCGAAG |
| | (p.Trp96Arg) | | | AATACCAGCCCACATACTGC′] |
| 1261 | NM_000330.3(RS1): c.38T > C | 6247 | RS1 | [′ATAGAAGGCTTTTTGTTATTACTTCYCTTTG |
| | (p.Leu13Pro) | | | GCTATGAAGGTATGTGCTAT′] |
| 1262 | NM_000330.3(RS1): c.667T > C | −1 | — | [′CCGAGCTGAGGCAGGCATCAGGCACRCTTG |
| | (p.Cys223Arg) | | | CTGACGCACTCCAGCAGCTCC′] |
| 1263 | NM_002764.3(PRPS1): c.344T > | 5631 | PRPS1 | [′ATCTCAGCCAAGCTTGTTGCAAATAYGCTAT |
| | C (p.Met115Thr) | | | CTGTAGCAGGTGCAGATCAT′] |
| 1264 | NM_002764.3(PRPS1): c.455T > | 5631 | PRPS1 | [′AATTTGTATGCAGAGCCGGCTGTCCYAAAGT |
| | C (p.Leu152Pro) | | | GGATAAGGGAGAATATCTCT′] |
| 1265 | NM_002764.3(PRPS1): c.869T > | 5631 | PRPS1 | [′TTTCCTTTCTTGCCTTTCTAGGTGAYTGACAT |
| | C (p.Ile290Thr) | | | CTCTATGATCCTTGCAGAA′] |
| 1266 | NM_000291.3(PGK1): c.263T > C | 5230 | PGK1 | [′CCAGTTGCTGTAGAACTCAAATCTCYGCTGG |
| | (p.Leu88Pro) | | | GCAAGTAAGTGCCAGGCTCT′] |
| 1267 | NM_000291.3(PGK1): c.946T > C | 5230 | PGK1 | [′CACCTCTACCCCTCAGGGCTTGGACYGTGGT |
| | (p.Cys316Arg) | | | CCTGAAAGCAGCAAGAAGTA′] |
| 1268 | NM_000291.3(PGK1): c.758T > C | 5230 | PGK1 | [′CATCATTTTGGCTCCCCTGTGTAGAYTGGCA |
| | (p.Ile253Thr) | | | CTTCTCTGTTTGATGAAGAG′] |
| 1269 | NM_001122606.1(LAMP2): | 3920 | LAMP2 | [′CATTGCAAATAACAATCTCAGCTACYGGGA |
| | c.961T > C (p.Trp321Arg) | | | TGCCCCCCTGGGAAGTTCTTA′] |
| 1270 | NM_000206.2(IL2RG): c.343T > | 3561 | IL2RG | [′ATTCTCTGAAGAAATCACTTCTGGCYGTCAG |
| | C (p.Cys115Arg) | | | TTGCAAAAAAAGGAGATCCA′] |
| 1271 | NM_000206.2(IL2RG): c.452T > | 3561 | IL2RG | [′ACACAGATGCTAAAACTGCAGAATCYGGGT |
| | C (p.Leu151Pro) | | | AATTTGGAAAGAAAGGGTCAA′] |
| 1272 | NM_000194.2(HPRT1): c.122T > | 3251 | HPRT1 | [′GAAAGGGTGTTTATTCCTCATGGACYAATTA |
| | C (p.Leu41Pro) | | | TGGACAGGTAAGTAAGATCT′] |
| 1273 | NM_000194.2(HPRT1): c.170T > | 3251 | HPRT1 | [′CTTGCTCGAGATGTGATGAAGGAGAYGGGA |
| | C (p.Met57Thr) | | | GGCCATCACATTGTAGCCCTC′] |
| 1274 | NM_000132.3(F8): c.6554T > C | 2157 | F8 | [′ATTCGCAGCACTCTTCGCATGGAGTYGATGG |
| | (p.Leu2185Ser) | | | GCTGTGATTTAAATAGTAAG′] |
| 1275 | NM_000132.3(F8): c.985T > C | 2157 | F8 | [′GGACCTTGGACAGTTTCTACTGTTTYGTCAT |
| | (p.Cys329Arg) | | | ATCTCTTCCCACCAACATGG′] |
| 1276 | NM_000132.3(F8): c.5372T > C | 2157 | F8 | [′AGAGCAGAAGTTGAAGATAATATCAYGGTG |
| | (p.Met1791Thr) | | | AGTTAAGGACAGTGGAATTAC′] |
| 1277 | NM_000132.3(F8): c.1754T > C | 2157 | F8 | [′CCTTTCAATATATGTAATTAACAGAYAATGT |
| | (p.Ile585Thr) | | | CAGACAAGAGGAATGTCATC′] |
| 1278 | NM_000132.3(F8): c.935T > C | 2157 | F8 | [′TCCTTGGAAATCTCGCCAATAACTTYCCTTA |
| | (p.Phe312Ser) | | | CTGCTCAAACACTCTTGATG′] |
| 1279 | NM_000132.3(F8): c.980T > C | 2157 | F8 | [′TTGATGGACCTTGGACAGTTTCTACYGTTTT |
| | (p.Leu327Pro) | | | GTCATATCTCTTCCCACCAA′] |
| 1280 | NM_000132.3(F8): c.1174T > C | 2157 | F8 | [′CTCTCCTTCCTTTATCCAAATTCGCYCAGTTG |
| | (p.Ser392Pro) | | | CCAAGAAGCATCCTAAAAC′] |
| 1281 | NM_000132.3(F8): c.1417T > C | 2157 | F8 | [′ATCAGGAATCTTGGGACCTTTACTTYATGGG |
| | (p.Tyr473His) | | | GAAGTTGGAGACACACTGTT′] |
| 1282 | NM_000132.3(F8): c.1481T > C | 2157 | F8 | [′AATCAAGCAAGCAGACCATATAACAYCTAC |
| | (p.Ile494Thr) | | | CCTCACGGAATCACTGATGTC′] |
| 1283 | NM_000132.3(F8): c.1729T > C | 2157 | F8 | [′CCCTCTCCTCATCTGCTACAAAGAAYCTGTA |
| | (p.Ser577Pro) | | | GATCAAAGAGGAAACCAGGT′] |
| 1284 | NM_000132.3(F8): c.1958T > C | 2157 | F8 | [′CAGTTGTCAGTTTGTTTGCATGAGGYGGCAT |
| | (p.Val653Ala) | | | ACTGGTACATTCTAAGCATT′] |
| 1285 | NM_000132.3(F8): c.2029T > C | 2157 | F8 | [′TTCTGTCTTCTTCTCTGGATATACCYTCAAAC |
| | | | | ACAAAATGGTCTATGAAGA′] |
| 1286 | NM_000132.3(F8): c.6193T > C | 2157 | F8 | [′GTTTCTTTACTTGGGCAAAGGACAGYGGGCC |
| | | | | CCAAAGCTGGCCAGACTTCA′] |
| 1287 | NM_007325.4(GRIA3): c.2117T > | 2892 | GRIA3 | [′GTGTACGAGAAAATGTGGTCTTACAYGAAA |
| | C (p.Met706Thr) | | | TCAGCGGAGCCATCTGTGTTT′] |
| 1288 | NM_000402.4(G6PD): c.1058T > | 2539 | G6PD | [′GAGGGCGAGGCCACCAAAGGGTACCYGGAC |
| | C (p.Leu353Pro) | | | GACCCCACGGTGCCCCGCGGG′] |
| 1289 | NM_000402.4(G6PD): c. 1054T > | 2539 | G6PD | [′TGGAGAGGGCGAGGCCACCAAAGGGYACCT |
| | C (p.Tyr352His) | | | GGACGACCCCACGGTGCCCCG′] |
| 1290 | NM_001097642.2(GJB1): | 2705 | GJB1 | [′GGTCCACATCTCAGGGACACTGTGGYGGAC |
| | c.397T > C (p.Trp133Arg) | | | CTATGTCATCAGCGTGGTGTT′] |
| 1291 | NM_000166.5(GJB1): c.407T > C | 2705 | GJB1 | [′TCAGGGACACTGTGGTGGACCTATGYCATC |
| | (p. Val136Ala) | | | AGCGTGGTGTTCCGGCTGTTG′] |
| 1292 | NM_000032.4(ALAS2): c.595T > | 212 | ALAS2 | [′TGTGTCCGTCTGGTGTAGTAATGATYACCTG |
| | C (p.Tyr199His) | | | GGCATGAGCCGACACCCTCA′] |
| 1293 | NM_020061.5(OPNILW): | 5956 | OPNILW | [′CTGGCCCCACGGCCTGAAGACTTCAYGCGG |
| | c.607T > C (p.Cys203Arg) | | | CCCAGACGTGTTCAGCGGCAG′] |
| 1294 | NM_000513.2(OPN1MW): | 2652 | OPN1MW | [′CTGGCCCCACGGCCTGAAGACTTCAYGCGG |
| | c.607T > C (p.Cys203Arg) | | | CCCAGACGTGTTCAGCGGCAG′] |
| 1295 | NM_000513.2(OPN1MW): | 2652 | OPN1MW | [′GGCCATCGTGGGCATTGCCTTCTCCYGGATC |
| | c.529T > C (p.Trp177Arg) | | | TGGGCTGCTGTGTGGACAGC′] |
| 1296 | NM_000273.2(GPR143): c.397T > | 4935 | GPR143 | [′GCTGTTGTACAGTGCCTGCTTCTGGHGGCTG |
| | C (p.Trp133Arg) | | | TTTTGCTATGCAGTGGATGC′] |
| 1297 | NM_000133.3(F9): c.52T > C | 2158 | F9 | [′AGAATCACCAGGCCTCATCACCATCYGCCTT |
| | (p.Cys18Arg) | | | TTAGGATATCTACTCAGTGC′] |
| 1298 | NM_000133.3(F9): c.1058T > C | 2158 | F9 | [′TTCCTCAAATTTGGATCTGGCTATGYAAGTG |
| | (p.Val353Ala) | | | GCTGGGGAAGAGTCTTCCAC′] |
| 1299 | NM_000133.3(F9): c.1144T > C | 2158 | F9 | [′AGTTCCACTTGTTGACCGAGCCACAHGTCTT |
| | (p.Cys382Arg) | | | CGATCTACAAAGTTCACCAT′] |
| 1300 | NM_000133.3(F9): c.1328T > C | 2158 | F9 | [′TGTGCAATGAAAGGCAAATATGGAAYATAT |
| | (p.Ile443Thr) | | | ACCAAGGTATCCCGGTATGTC′] |
| 1301 | NM_000133.3(F9): c.1357T > C | 2158 | F9 | [′TACCAAGGTATCCCGGTATGTCAACYGGATT |
| | (p.Trp453Arg) | | | AAGGAAAAAACAAAGCTCAC′] |
| 1302 | NM_000133.3(F9): c.82T > C | 2158 | F9 | [′TTTAGGATATCTACTCAGTGCTGAAYGTACA |
| | (p.Cys28Arg) | | | GGTTTGTTTCCTTTTTTAAA′] |
| 1303 | NM_000133.3(F9): | 2158 | F9 | [′CTGAATTTTGGAAGCAGTATGTTGGYAAGC |
| | c.277 + 2T > C | | | AATTCATTTTATCCTCTAGCT′] |
| 1304 | NM_000133.3(F9): c.1031T > C | 2158 | F9 | [′ATTGCTGACAAGGAATACACGAACAYCTTC |
| | (p.Ile344Thr) | | | CTCAAATTTGGATCTGGCTAT′] |
| 1305 | NM_000169.2(GLA): c.484T > C | −1 | — | [′CATTGATGCCCAGACCTTTGCTGACYGGGGA |
| | (p.Trp162Arg) | | | GTAGATCTGCTAAAATTTGA′] |
| 1306 | NM_000169.2(GLA): c.806T > C | −1 | — | [′TTATTTCATTCTTTTTCTCAGTTAGYGATTGG |
| | (p.Val269Ala) | | | CAACTTTGGCCTCAGCTGG′] |
| 1307 | NM_015107.2(PHF8): c.836T > C | 23133 | PHF8 | [′CCAACAAATGCCAATCTGACTCTCTYTGAGT |
| | (p.Phe279Ser) | | | GCTGGAGCAGTTCCTCTAAT′] |
| 1308 | NM_033290.3(MID1): c.1877T > | 4281 | MID1 | [′TATGATGCTTTGAACTCCATCCACCYCTACA |
| | C (p.Leu626Pro) | | | CCTTCGACGTCGCATTTGCG′] |
| 1309 | NM_033290.3(MID1): c.884T > C | 4281 | MID1 | [′TTCAAGGTGATGAGGCTTCGCAAACYGGCT |
| | (p.Leu295Pro) | | | CAGCAGATTGCAAACTGCAAA′] |
| 1310 | NM_000444.5(PHEX): c.755T > C | 5251 | PHEX | [′CAGTATCGGGATGCCCTTTACAAGTYCATGG |
| | (p.Phe252Ser) | | | TGGATACTGCCGTGCTTTTA′] |
| 1311 | NM_000444.5(PHEX): c. 1664T > | 5251 | PHEX | [′CTCATAGGATTTCCAGCAGGAGAGCYCCAG |
| | C (p.Leu555Pro) | | | AAGCCTTTCTTTTGGGGAACA′] |
| 1312 | NM_001205019.1(GK): c.1525T > | 2710 | GK | [′AGAAAGTGAAATTCGTTATTCTACAYGGAA |
| | C (p.Trp509Arg) | | | GAAAGCTGTGATGAAGTCAAT′] |
| 1313 | NM_000475.4(NROB1): c.890T > | 190 | NROB1 | [′AACTGCTGGGCGTCCCTGCTCATGCYTGAGC |
| | C (p.Leu297Pro) | | | TGGCCCAGGACCGCTTGCAG′] |
| 1314 | NM_000531.5(OTC): c.332T > C | 5009 | OTC | [′CTTCTGGGAGGACATCCTTGTTTTCYTACCA |
| | (p.Leul11Pro) | | | CACAAGATATTCATTTGGGT′] |
| 1315 | NM_000531.5(OTC): c.134T > C | 5009 | OTC | [′CTGAAGGGCCGTGACCTTCTCACTCYAAAA |
| | (p.Leu45Pro) | | | AACTTTACCGGAGAAGAAATT′] |
| 1316 | NM_000531.5(OTC): | 5009 | OTC | [′TTGGCAGAGCAGTATGCCAAAGAGGYATGC |
| | c.717 + 2T > C | | | TCTTTACATGTAAAGCTATTA′] |
| 1317 | NM_001399.4(EDA): c.181T > C | 1896 | EDA | [′CCTCCACCTGCTGACGTTGTGCTGCYACCTA |
| | (p.Tyr61His) | | | GAGTTGCGCTCGGAGTTGCG′] |
| 1318 | NM_001015877.1(PHF6): c.2T > | 84295 | PHF6 | [′CATTCTAAAGGCAATTTAAAAATCAYGTCA |
| | C (p.Met1Thr) | | | AGCTCAGTTGAACAGAAAAAA′] |
| 1319 | NM_001128834.2(PLP1): | 5354 | PLP1 | [′CATCACCTATGCCCTGACCGTTGTGYGGCTC |
| | c.487T > C (p.Trp163Arg) | | | CTGGTGTTTGCCTGCTCTGC′] |
| 1320 | NM_001128834.2(PLP1): | 5354 | PLP1 | [′CCTGGCAAGGTTTGTGGCTCCAACCYTCTGT |
| | c.671T > C (p.Leu224Pro) | | | CCATCTGCAAAACAGCTGAG′] |
| 1321 | NM_001128834.2(PLP1): | 5354 | PLP1 | [′AACACCTGGACCACCTGCCAGTCTAYTGCCT |
| | c.560T > C (p.Ile187Thr) | | | TCCCCAGCAAGACCTCTGCC′] |
| 1322 | NM_001128834.2(PLP1): | 5354 | PLP1 | [′ATTTTCCTGCAGTTCCAAATGACCTYCCACC |
| | c.710T > C (p.Phe237Ser) | | | TGTTTATTGCTGCATTTGTG′] |
| 1323 | NM_000116.4(TAZ): c.352T > C | 6901 | TAZ | [′CTCCCACTTCTTCAGCTTGGGCAAGYGTGTG |
| | (p.Cys118Arg) | | | CCTGTGTGCCGAGGTGAGCT′] |
| 1324 | NM_000377.2(WAS): c.244T > C | 7454 | WAS | [′CTTCGTGAAGGATAACCCCCAGAAGYCCTA |
| | (p.Ser82Pro) | | | CTTCATCCGCCTTTACGGCCT′] |
| 1325 | NM_000377.2(WAS): c.809T > C | 7454 | WAS | [′AACCTCGACCCAGATCTGCGGAGTCYGTTCT |
| | (p.Leu270Pro) | | | CCAGGGCAGGAATCAGCGAG′] |
| 1326 | NM_182680.1(AMELX): c.2T > C | −1 | — | [′CTTACATTTCAGAACCATCAAGAAAYGGGG |
| | (p.Met1Thr) | | | ACCTGGATTTTATTTGCCTGC′] |
| 1327 | NM_000074.2(CD40LG): | 959 | CD40LG | [′ACCATGAGCAACAACTTGGTAACCCYGGAA |
| | c.464T > C (p.Leu155Pro) | | | AATGGGAAACAGCTGACCGTT′] |
| 1328 | NM_139058.2(ARX): c.98T > C | 170302 | ARX | [′TCCTCCTACTGCATCGACAGCATCCYGGGCC |
| | (p.Leu33Pro) | | | GGAGGAGCCCGTGCAAAATG′] |
| 1329 | NM_001109878.1(TBX22): | 50945 | TBX22 | [′TGATCATTTCTCCTCCAGATCATTCYGCAAT |
| | c.641T > C (p.Leu214Pro) | | | CCATGCATAAGTACAAACCC′] |
| 1330 | NM_000061.2(BTK): c.2T > C | 695 | BTK | [′GGTGAACTCCAGAAAGAAGAAGCTAYGGCC |
| | (p.Met1Thr) | | | GCAGTGATTCTGGAGAGCATC′] |
| 1331 | NM_000061.2(BTK): c.1223T > C | 695 | BTK | [′AAGGACCTGACCTTCTTGAAGGAGCYGGGG |
| | (p.Leu408Pro) | | | ACTGGACAATTTGGGGTAGTG′] |
| 1332 | NM_000061.2(BTK): c.1516T > C | 695 | BTK | [′GCTGCTAGAGATGTGCAAGGATGTCYGTGA |
| | (p.Cys506Arg) | | | AGCCATGGAATACCTGGAGTC′] |
| 1333 | NM_000061.2(BTK): c.1625T > C | 695 | BTK | [′GTTGTTAAAGTATCTGATTTCGGCCYGTCCA |
| | (p.Leu542Pro) | | | GGTGAGTGTGGCTTTTTCAT′] |
| 1334 | NM_000061.2(BTK): c.1741T > C | 695 | BTK | [′CAAGTTCAGCAGCAAATCTGACATTYGGGC |
| | (p.Trp581Arg) | | | TTTTGGTAAGTGGATAAGATT′] |
| 1335 | NM_000061.2(BTK): c.1955T > C | 695 | BTK | [′TTCAAAATTCTTCTGAGCAATATTCYAGATG |
| | (p.Leu652Pro) | | | TCATGGATGAAGAATCCTGA′] |
| 1336 | NM_015884.3(MBTPS2): | 51360 | MBTPS2 | [′GATGGACAATGGATTCTAAACTCTTYCTTGG |
| | c.1424T > C (p.Phe475Ser) | | | ATGCCACCCTTACCTCAGTG′] |
| 1337 | NM_014009.3(FOXP3): c.970T > | 50943 | FOXP3 | [′GATTCATCCCCACCCTCTGACAGAGYTCCTC |
| | C (p.Phe324Leu) | | | CACAACATGGACTACTTCAA′] |
| 1338 | NM_014009.3(FOXP3): | 50943 | FOXP3 | [′GACACTCAATGAGATCTACCACTGGYTCAC |
| | c.1099T > C (p.Phe367Leu) | | | ACGCATGTTTGCCTTCTTCAG′] |
| 1339 | NM_022567.2(NYX): c.302T > C | 60506 | NYX | [′CTGCGCCACAACAACCTGTCCTTCAYCACGC |
| | (p.Ile101Thr) | | | CCGGCGCCTTCAAGGGCCTG′] |
| 1340 | NM_003639.4(IKBKG): | 8517 | IKBKG | [′CACCCTGCAGATACATGTCATGGAGYGCATT |
| | c.1249T > C (p.Cys417Arg) | | | GAGTAGGGCCGGCCAGTGCA′] |
| 1341 | NM_006579.2(EBP): c.53T > C | 10682 | EBP | [′CCATACTGGCCTCAGCACCTAAGACYGGAC |
| | (p.Leu18Pro) | | | AACTTTGTACCTAATGACCGC′] |
| 1342 | NM_003159.2(CDKL5): c.215T > | 6792 | CDKL5 | [′CTTCGGACTCTCAAGCAGGAAAACAHTGTG |
| | C (p.Ile72Thr) | | | GAGTTGAAGGAAGCATTTCGT′] |
| 1343 | NM_001011658.3(TRAPPC2): | −1 | — | [′GCCTTAATTATTTCACATATGAGGTYTATTA |
| | c.248T > C (p.Phe83Ser) | | | TGCTTCATGACATAAGACAA′] |
| 1344 | NM_001011658.3(TRAPPC2): | −1 | — | [′AGTTACCTTTACTAAGAAGGTAAGGRTATGC |
| | 238 + 4T > C | | | CCCGCAGTGACAAATGCCGA′] |
| 1345 | NM_003688.3(CASK): c.802T > C | 8573 | CASK | [′GGATCCAGCTGAAAGGATCACTGTTYATGA |
| | (p.Tyr268His) | | | AGCACTGAATCACCCATGGCT′] |
| 1346 | NM_003688.3(CASK): c.2740T > | 8573 | CASK | [′TGAGCTCGTGTGCACAGCCCCACAGYGGGT |
| | C (p.Trp914Arg) | | | CCCTGTCTCCTGGGTCTATTA′] |
| 1347 | NM_001159702.2(FHL1): | 2273 | FHL1 | [′AGGGGAGGACTTCTACTGCGTGACTYGCCA |
| | c.457T > C (p.Cys153Arg) | | | TGAGACCAAGTTTGCCAAGCA′] |
| 1348 | NM_001159702.2(FHL1): | 2273 | FHL1 | [′GGAGGACTCCCCCAAGTGCAAGGGGYGCTT |
| | c.310T > C (p.Cys104Arg) | | | CAAGGCCATTGTGGCAGGTAC′] |
| 1349 | NM_001159702.2(FHL1): | 2273 | FHL1 | [′CACCGCTGTGGAGGACCAGTATTACYGCGT |
| | c.625T > C (p.Cys209Arg) | | | GGATTGCTACAAGAACTTTGT′] |
| 1350 | NM_001363.4(DKC1): c.113T > C | 1736 | DKC1 | [′ATACAACACGCTGAAGAATTTCTTANCAAA |
| | (p.Ile38Thr) | | | CCTGAATCCAAAGTTGCTAAG′] |
| 1351 | NM_178152.2(DCX): c.373T > C | 1641 | DCX | [′CTTATTTCTTGCCTTAGGGGAAAGCYATGTC |
| | (p.Tyr125His) | | | TGTTCCTCAGACAACTTCTT′] |
| 1352 | NM_005183.3(CACNA1F): | 778 | CACNAIF | [′ATCCTGTTGAACGTGTTTCTTGCCAYTGCTG |
| | c.2267T > C (p.Ile756Thr) | | | TGGACAACCTGGCCAGTGGA′] |
| 1353 | NM_001493.2(GDI1): c.275T > C | 2664 | GDI1 | [′ACAGGGCAGCTGGTAAAGATGCTACYGTAT |
| | (p.Leu92Pro) | | | ACAGAGGTGACTCGCTACCTG′] |
| 1354 | NM_006517.4(SLC16A2): | 6567 | SLC16A2 | [′ATCATGGCCCCCATTGCATTTGAGCYGGTGG |
| | c.1313T > C (p.Leu438Pro) | | | GCCCAATGCAGGCCTCACAG′] |
| 1355 | NM_006517.4(SLC16A2): | 6567 | SLC16A2 | [′CTCCAGGTCCTTTCCTTCCTGCTCCYGGGCC |
| | c.1190T > C (p.Leu397Pro) | | | TGATGTCCATGATGATTCCC′] |
| 1356 | NM_006517.4(SLC16A2): | 6567 | SLC16A2 | [′CCCCCCATCATCGGGGCTGTAATCCYCTTCT |
| | c.1481T > C (p.Leu494Pro) | | | TCGTCCCTCTGATGCATCAA′] |
| 1357 | NM_004586.2(RPS6KA3): | 6197 | RPS6KA3 | [′GAAATGCTTACTGGTACACTCCCTTYCCAAG |
| | c.803T > C (p.Phe268Ser) | | | GAAAAGATCGAAAAGAAACA′] |
| 1358 | NM_000489.4(ATRX): c.4840T > | 546 | ATRX | [′AAGTTTTCTTCATACAGTTCTTTTGYGTGAC |
| | C (p.Cys1614Arg) | | | AAACTGGATTTCAGCACGGC′] |
| 1359 | NM_000489.4(ATRX): c.6250T > | 546 | ATRX | [′GAAGTGGCTTCGAAACATTGACTATYACCGT |
| | C (p.Tyr2084His) | | | TTAGATGGTTCCACTACTGC′] |
| 1360 | NM_000489.4(ATRX): c.1226T > | 546 | ATRX | [′GATATTAAGAAGGCTCATCTTGCATYGGAA |
| | C (p.Leu409Ser) | | | GAAGACTTAAATTCCGAGTTT′] |
| 1361 | NM_000489.4(ATRX): c.6149T > | 546 | ATRX | [′CAGTCCCTCATATCTCTGGACTTGAYTGAAG |
| | C (p.Ile2050Thr) | | | ATTTTCTTGAATTAGCTAGT′] |
| | NM_001110556.1(FLNA): | 2316 | FLNA | |
| | c.720 + 2T > C | | | |
| 1362 | NM_001127899.3(CLCN5): | 1184 | CLCN5 | [′AGGTGGGGTGACTCGGATGACTGTTYCTCTT |
| | 6c.178T > C (p.Ser590Pro) | | | GTTGTCATAATGTTTGAACT′] |
| 1363 | NM_004992.3(MECP2): c.464T > | 4204 | MECP2 | [′GACACATCCCTGGACCCTAATGATTBTGACT |
| | C (p.Phe155Ser) | | | TCACGGTAACTGGGAGAGGG′] |
| 1364 | NM_001061.4(TBXAS1): | 6916 | TBXAS1 | [′TGCCTCGGGGTGCGTCTAGGGCTGCYTGAG |
| | c.1463T > C (p.Leu488Pro) | | | GTCAAGTTGACACTGCTCCAC′] |
| 1365 | NM_001061.4(TBXAS1): | 6916 | TBXAS1 | [′TTTTTTATTCCTCCCAGGTACTATCYTGGTCG |
| | c.248T > C (p.Leu83Pro) | | | TCGGATGTTTATTGTTATT′] |
| 1366 | NM_000128.3(F11): c.901T > C | 2160 | F11 | [′TTCTTCATTTTACCATGACACTGATYTCTTGG |
| | (p.Phe301Leu) | | | GAGAAGAACTGGATATTGT′] |
| 1367 | NM_000128.3(F11): c.166T > C | 2160 | F11 | [′GGTAGTCTGCACTTACCACCCAAGAYGTTTA |
| | (p.Cys56Arg) | | | CTCTTCACTTTCACGGCGGA′] |
| 1368 | NM_000203.4(IDUA): c.1469T > | 3425 | IDUA | [′AGCCCCGACGGCGAGTGGCGGCGCCYGGGC |
| | C (p.Leu490Pro) | | | CGGCCCGTCTTCCCCACGGCA′] |
| 1369 | NM_000431.3(MVK): c.803T > C | 4598 | MVK | [′ATCGTGGCCCCCCTCCTGACCTCAAYAGATG |
| | (p.Ile268Thr) | | | CCATCTCCCTGGAGTGTGAG′] |
| 1370 | NM_014425.3(INVS): c.1478T > | 27130 | INVS | [′TTATGCTTATAGGGAAGAACAGCTTYGCATT |
| | C (p.Leu493Ser) | | | GGTCCTGCAACAATGGATAC′] |
| 1371 | NM_000108.4(DLD): c.1178T > C | 1738 | DLD | [′GACTACAATTGTGTGCCATCAGTGAYTTACA |
| | (p.Ile393Thr) | | | CACACCCTGAAGTTGCTTGG′] |
| 1372 | NM_000170.2(GLDC): c.2T > C | 2731 | GLDC | [′GGGCAGGGGACGGTGGCCGCGGCCAYGCAG |
| | (p.Met1Thr) | | | TCCTGTGCCAGGGCGTGGGGG′] |
| 1373 | NM_000151.3(G6PC): c.229T > C | 2538 | G6PC | [′AGACTGGCTCAACCTCGTCTTTAAGYGGTAA |
| | (p.Trp77Arg) | | | GAACCATATAGAGAGGAGAT′] |
| 1374 | NM_004453.3(ETFDH): c.2T > C | 2110 | ETFDH | [′CCGAGAGTCCTGGTGACTTTGAACAYGCTG |
| | (p.Met1Thr) | | | GTGCCGCTAGCCAAGCTGTCC′] |
| 1375 | NM_000136.2(FANCC): | −1 | — | [′CTGGCCCGAGAGCTCCTTAAAGAGCYGCGA |
| | c.1661T > C (p.Leu554Pro) | | | ACTCAAGTCTAGAAGGCACGC′] |
| 1376 | NM_000131.4(F7): c.38T > C | 2155 | F7 | [′GCCCTCAGGCTCCTCTGCCTTCTGCYTGGGC |
| | (p.Leu13Pro) | | | TTCAGGGCTGCCTGGCTGCA′] |
| 1377 | NM_000131.4(F7): c.983T > C | 2155 | F7 | [′CCCCTCTGCCTGCCCGAACGGACGTYCTCTG |
| | (p.Phe328Ser) | | | AGAGGACGCTGGCCTTCGTG′] |
| 1378 | NM_024649.4(BBS1): c.1553T > | −1 | — | [′ACAACCCGTCCTGTCCTGGGGCTGCYGGTCT |
| | C (p.Leu518Pro) | | | GCTTCCTGTACAACGAGGCG′] |
| 1379 | NM_000490.4(AVP): c.200T > C | 551 | AVP | [′TGCGCGGACGAGCTGGGCTGCTTCGYGGGC |
| | (p.Val67Ala) | | | ACGGCTGAGGCGCTGCGCTGC′] |
| 1380 | NM_000490.4(AVP): c.61T > C | 551 | AVP | [′CCTACTGGCCTTCTCCTCCGCGTGCYACTTC |
| | (p.Tyr21His) | | | CAGAACTGCCCGAGGGGCGG′] |
| 1381 | NM_001692.3(ATP6V1B1): | 525 | ATP6V1B1 | [′GGGACTCAGAGGAGCGGGCAGGTGCYTGAG |
| | c.242T > C (p.Leu81Pro) | | | GTGGCTGGCACCAAGGCGATT′] |
| 1382 | NM_003361.3(UMOD): c.649T > | 7369 | UMOD | [′GGGCGGTGCGCGCATGGCCGAGACCBGCGT |
| | C (p.Cys217Arg) | | | GCCAGTCCTGCGCTGCAACAC′] |
| 1383 | NM_003361.3(UMOD): c.376T > | 7369 | UMOD | [′TAGCCACTGCCACGCCCTGGCCACAYGTGTC |
| | C (p.Cys126Arg) | | | AATGTGGTGGGCAGCTACTT′] |
| 1384 | NM_003361.3(UMOD): c.943T > | 7369 | UMOD | [′CAAATCGAATAATGGCAGATGGCACYGCCA |
| | C (p.Cys315Arg) | | | GTGCAAACAGGACTTCAACAT′] |
| 1385 | NM_080911.2(UNG): c.752T > C | 7374 | UNG | [′AATCAGAACTCGAATGGCCTTGTTTYCTTGC |
| | (p.Phe251Ser) | | | TCTGGGGCTCTTATGCTCAG′] |
| 1386 | NM_199292.2(TH): c.707T > C | 7054 | TH | [′CAGGTGTACCGCCAGCGCAGGAAGCYGATT |
| | (p.Leu236Pro) | | | GCTGAGATCGCCTTCCAGTAC′] |
| 1387 | NM_001065.3(TNFRSF1A): | 7132 | TNFRSF1A | [′CCACCCTCAAAATAATTCGATTTGCYGTACC |
| | c.175T > C (p.Cys59Arg) | | | AAGTGCCACAAAGGTAGGGG′] |
| 1388 | NM_001065.3(TNFRSF1A): | 7132 | TNFRSF1A | [′AATGGGTCAGGTGGAGATCTCTTCTYGCACA |
| | c.349T > C (p.Cys117Arg) | | | GTGGACCGGGACACCGTGTG′] |
| 1389 | NM_000546.5(TP53): c.755T > C | 7157 | TP53 | [′GGCGGCATGAACCGGAGGCCCATCCYCACC |
| | (p.Leu252Pro) | | | ATCATCACACTGGAAGACTCC′] |
| 1390 | NM_000546.5(TP53): c.398T > C | 7157 | TP53 | [′CAGTACTCCCCTGCCCTCAACAAGAYGTTTT |
| | (p.Met133Thr) | | | GCCAACTGGCCAAGACCTGC′] |
| 1391 | NM_000546.5(TP53): c.1031T > C | 7157 | TP53 | [′GAGCGCTTCGAGATGTTCCGAGAGCYGAAT |
| | (p.Leu344Pro) | | | GAGGCCTTGGAACTCAAGGAT′] |
| 1392 | NM_001018005.1(TPM1): | 7168 | TPM1 | [′TCTCTGAACAGACGCATCCAGCTGGYTGAG |
| | c.284T > C (p. Val95Ala) | | | GAAGAGTTGGATCGTGCCCAG′] |
| 1393 | NM_001159287.1(TPI1): | 7167 | TPI1 | [′GGGTGGTGCTTCCCTCAAGCCCGAAYTCGTG |
| | c.832T > C (p.Phe278Leu) | | | GACATCATCAATGCCAAACA′] |
| 1394 | NM_003242.5(TGFBR2): | 7048 | TGFBR2 | [′AAGCATGAGAACATACTCCAGTTCCYGACG |
| | c.923T > C (p.Leu308Pro) | | | GCTGAGGAGCGGAAGACGGAG′] |
| 1395 | NM_000660.5(TGFB1): c.673T > | 7040 | TGFB1 | [′CTTTCGCCTTAGCGCCCACTGCTCCYGTGAC |
| | C (p.Cys225Arg) | | | AGCAGGGATAACACACTGCA′] |
| 1396 | NM_000660.5(TGFB1): c.241T > | 7040 | TGFB1 | [′GCTGCCCGAGGCCGTGCTCGCCCTGYACAA |
| | C (p.Tyr81His) | | | CAGCACCCGCGACCGGGTGGC′] |
| 1397 | NM_000660.5(TGFB1): c.667T > | 7040 | TGFB1 | [′TGAGGGCTTTCGCCTTAGCGCCCACNGCTCC |
| | C (p.Cys223Arg) | | | TGTGACAGCAGGGATAACAC′] |
| 1398 | NM_001128177.1(THRB): | 7068 | THRB | [′ATCATCCTCCTCAAAGGCTGCTGCAYGGAG |
| | c.929T > C (p.Met310Thr) | | | ATCATGTCCCTTCGCGCTGCT′] |
| 1399 | NM_001128177.1(THRB): | 7068 | THRB | [′CCGCTTCCTGCACATGAAGGTGGAAYGCCC |
| | c.1336T > C (p.Cys446Arg) | | | CACAGAACTCTTCCCCCCTTT′] |
| 1400 | NM_001128177.1(THRB): | 7068 | THRB | [′CTCTTCCCCCCTTTGTTCTTGGAAGYGTTCG |
| | 3c.137T > C (p.Val458Ala) | | | AGGATTAGACTGACTGGATT′] |
| 1401 | NM_007313.2(ABL1): c.814T > C | 25 | ABL1 | [′GAAGCACAAGCTGGGCGGGGGCCAGYACGG |
| | (p.Tyr272His) | | | GGAGGTGTACGAGGGCGTGTG′] |
| 1402 | NM_007313.2(ABL1): c.988T > C | 25 | ABL1 | [′AGGGGTCTGCACCCGGGAGCCCCCGYTCTA |
| | (p.Phe330Leu) | | | TATCATCACTGAGTTCATGAC′] |
| 1403 | NM_007313.2(ABL1): c.1109T > | 25 | ABL1 | [′ATGGCCACTCAGATCTCGTCAGCCAYGGAG |
| | C (p.Met370Thr) | | | TACCTGGAGAAGAAAAACTTC′] |
| 1404 | NM_021961.5(TEAD1): | 7003 | TEAD1 | [′TGAACACGGAGCACAACATCATATTYACAG |
| | c.1261T > C (p.Tyr?His) | | | GCTTGTAAAGGACTGAACATG′] |
| 1405 | NM_001256850.1(TTN): | 7273 | TTN | [′TGGATACCCATCCCCGACAGTGACAHGGTA |
| | c.2926T > C (p.Trp976Arg) | | | CAGGGAAGACTACCAAATCGA′] |
| 1406 | NM_133378.4(TTN): c.100163T > | −1 | — | [′GAAAACACAGATGACCTGACAACCCYGATC |
| | C (p.Leu33388Pro) | | | ATCATGGACGTACAGAAACAA′] |
| 1407 | NM_003235.4(TG): c.3733T > C | 7038 | TG | [′CCCCACAGGCTCTGCCATGCAGCAGYGCCA |
| | (p.Cys1245Arg) | | | ATTGCTGTGCCGCCAGGGCTC′] |
| 1408 | NM_003235.4(TG): c.3229T > C | 7038 | TG | [′TCTCTCTCCCACAGGCCCGACAACCYGCGAG |
| | (p.Cys1077Arg) | | | AAATCTCGAACCAGTGGGCT′] |
| 1409 | NM_000733.3(CD3E): | 916 | CD3E | [′CGGGTGCTGGCGGCAGGCAAAGGGGYAAGG |
| | c.520 + 2T > C | | | CTGTGGAGTCCAGTCAGAGGA′] |
| 1410 | NM_003000.2(SDHB): c.487T > C | 6390 | SDHB | [′GCCTTATTTGAAGAAGAAGGATGAAYCTCA |
| | (p.Ser163Pro) | | | GGAAGGCAAGCAGCAGTATCT′] |
| 1411 | NM_003106.3(SOX2): c.290T > C | −1 | — | [′CCGTTCATCGACGAGGCTAAGCGGCYGCGA |
| | (p.Leu97Pro) | | | GCGCTGCACATGAAGGAGCAC′] |
| 1412 | NM_001024858.2(SPTB): | 6710 | SPTB | [′TAATGTCACCAACTTTACCTCCAGCYGGAAG |
| | c.604T > C (p.Trp202Arg) | | | GATGGCTTGGCCTTTAATGC′] |
| 1413 | NM_001024858.2(SPTB): | 6710 | SPTB | [′GGTGTGCCAGTTCTCGAGGGATGCCYCTGTG |
| | c.6055T > C (p.Ser2019Pro) | | | GCTGAGGCGTGGCTGATTGC′] |
| 1414 | NM_003126.2(SPTA1): c.779T > | 6708 | SPTA1 | [′TTGGCTCTCCAGAGACAGAAAGCTCBGTCCA |
| | C (p.Leu260Pro) | | | ATGCTGCAAACTTACAACGA′] |
| 1415 | NM_003126.2(SPTA1): c.781T > | 6708 | SPTA1 | [′GGCTCTCCAGAGACAGAAAGCTCTGYCCAA |
| | C (p.Ser261Pro) | | | TGCTGCAAACTTACAACGATT′] |
| 1416 | NM_003126.2(SPTA1): c.620T > | 6708 | SPTA1 | [′AAATTTGAAGACTTCCAAGTGGAGCYGGTA |
| | C (p.Leu207Pro) | | | GCTAAAGAAGGGAGAGTTGTT′] |
| 1417 | NM_005633.3(SOS1): c.1294T > | 6654 | SOS1 | [′CGAGATTCAGAAGAATATTGATGGTYGGGA |
| | C (p.Trp432Arg) | | | GGGAAAAGACATTGGACAGTG′] |
| 1418 | NM_006920.4(SCN1A): | −1 | — | [′TGTGGAAGAAGGCAGAGGAAAACAAYGGT |
| | c.3577T > C (p.Trp1193Arg) | | | GGAACCTGAGAAGGACGTGTTT′] |
| 1419 | NM_006920.4(SCN1A): | −1 | — | [′TTTGGGTATCTCTCTTTGCTTCAAGYTGTAA |
| | c.4250T > C (p.Val1417Ala) | | | GTGAACACTATTTTCTCTGA′] |
| 1420 | NM_006920.4(SCN1A): c.434T > | 6323 | SCN1A | [′ACTATTTTGACAAACTGTGTGTTTAHGACAA |
| | C (p.Met145Thr) | | | TGAGTAACCCTCCTGATTGG′] |
| 1421 | NM_006920.4(SCN1A): | −1 | — | [′TCTATACTTTGGAGGTCAAGACATCYTTATG |
| | c.4462T > C (p.Phe1488Leu) | | | ACAGAAGAACAGAAGAAATA′] |
| 1422 | NM_152296.4(ATP1A3): | 478 | ATP1A3 | [′GGGCTGGAGGTGGGCAAGACGCCCAHCGCC |
| | c.821T > C (p.Ile274Thr) | | | ATCGAGATTGAGCACTTCATC′] |
| 1423 | NM_152296.4(ATP1A3): | 478 | ATP1A3 | [′CAGCAATATCCCGGAGATCACGCCCYTCCTG |
| | c.2338T > C (p.Phe780Leu) | | | CTGTTCATCATGGCCAACAT′] |
| 1424 | NM_000702.3(ATP1A2): | 477 | ATP1A2 | [′CCTCAGAATCTCCCCACAGGCCGCCYGATCT |
| | c.2291T > C (p.Leu764Pro) | | | TTGACAACTTGAAGAAATCC′] |
| 1425 | NM_000702.3(ATP1A2): | 477 | ATP1A2 | [′ACGGCTACTGGGAATCCGCCTCGACYGGGA |
| | c.2659T > C (p.Trp887Arg) | | | TGACCGGACCATGAATGATCT′] |
| 1426 | NM_000702.3(ATP1A2): | 477 | ATP1A2 | [′AAGAAGGCTGACATTGGCATTGCCAYGGGC |
| | c.2192T > C (p.Met731Thr) | | | ATCTCTGGCTCTGACGTCTCT′] |
| 1427 | NM_000702.3(ATP1A2): | 477 | ATP1A2 | [′GGGCGGACACCCATAGCAATGGAGAYTGAA |
| | c.857T > C (p.Ile286Thr) | | | CACTTCATCCAGCTGATCACA′] |
| 1428 | NM_001035.2(RYR2): c.1298T > | 6262 | RYR2 | [′ATGTTTATGGTTTATTTTAGGGGCCYTGATG |
| | C (p.Leu433Pro) | | | CTCTCAGCAAGAAAGCGAAG′] |
| 1429 | NM_000540.2(RYR1): | 6261 | RYR1 | [′GTGGGTGTCCGGGCTGGCGGAGGCAYTGGG |
| | c.14693T > C (p.Ile4898Thr) | | | GACGAGATCGAGGACCCCGCG′] |
| 1430 | NM_000539.3(RHO): c.133T > C | 6010 | RHO | [′GTTCTCCATGCTGGCCGCCTACATGYTTCTG |
| | (p.Phe45Leu) | | | CTGATCGTGCTGGGCTTCCC′] |
| 1431 | NM_000321.2(RB1): | 5925 | RB1 | [′CTCTTTCACTGTTTTATAAAAAAGGYTAGTA |
| | c.1960 + 2T > C | | | GATGATTATTTTCAAGAGCA′] |
| 1432 | NM_000321.2(RB1): c.2134T > C | 5925 | RB1 | [′TATGATGTGTTCCATGTATGGCATAYGCAAA |
| | (p.Cys712Arg) | | | GTGAAGAATATAGACCTTAA′] |
| 1433 | NM_000329.2(RPE65): c.1022T > | 6121 | RPE65 | [′AGATTTGAGTTTGTTTATAATTACTYATATTT |
| | C (p.Leu341Ser) | | | AGCCAATTTACGTGAGAAC′] |
| 1434 | NM_000322.4(PRPH2): c.554T > | 5961 | PRPH2 | [′ATTCAGTGGATCAGCAATCGCTACCYGGACT |
| | C (p.Leu185Pro) | | | TTTCCTCCAAAGAAGTCAAA′] |
| 1435 | NM_000322.4(PRPH2): c.2T > C | 5961 | PRPH2 | [′CAACCCGGACTACACTTGGCAAGCAYGGCG |
| | (p.Met1Thr) | | | CTACTGAAAGTCAAGTTTGAC′] |
| 1436 | NM_001098668.2(SFTPA2): | 729238 | SFTPA2 | [′ACTGAGGGTCCCAGCCCTGGAGACTYCCGC |
| | c.593T > C (p.Phe198Ser) | | | TACTCAGATGGGACCCCTGTA′] |
| 1437 | NM_003018.3(SFTPC): c.218T > | 6440 | SFTPC | [′TTTCCCCAGGTTCTGGAGATGAGCAHTGGGG |
| | C (p.Ile73Thr) | | | CGCCGGAAGCCCAGCAACGC′] |
| 1438 | NM_003018.3(SFTPC): c.581T > | 6440 | SFTPC | [′AGCACCCTGTGTGGCGAGGTGCCGCYCTACT |
| | C (p.Leu194Pro) | | | ACATCTAGGACGCCTCCGGT′] |
| 1439 | NM_000055.2(BCHE): c.1004T > | 590 | BCHE | [′CTCACTGACATGCCAGACATATTACYTGAAC |
| | C (p.Leu335Pro) | | | TTGGACAATTTAAAAAAACC′] |
| 1440 | NM_002739.3(PRKCG): c.355T > | 5582 | PRKCG | [′CCCCACCTTCTGCGACCACTGTGGCYCCCTC |
| | C (p.Ser119Pro) | | | CTCTACGGGCTTGTGCACCA′] |
| 1441 | NM_002739.3(PRKCG): | 5582 | PRKCG | [′ACAGTGTGGCCGCAGCGGCGAGAACYTTGA |
| | c.1927T > C (p.Phe643Leu) | | | CAAGTTCTTCACGCGGGCGGC′] |
| 1442 | NM_000141.4(FGFR2): | 2263 | FGFR2 | [′TGTAACTTTTGAGGACGCTGGGGAAYATAC |
| | c.1018T > C (p.Tyr340His) | | | GTGCTTGGCGGGTAATTCTAT′] |
| 1443 | NM_000141.4(FGFR2): | 2263 | FGFR2 | [′TTTTGAGGACGCTGGGGAATATACGHGCTTG |
| | c.1024T > C (p.Cys342Arg) | | | GCGGGTAATTCTATTGGGAT′] |
| 1444 | NM_000141.4(FGFR2): c.868T > | 2263 | FGFR2 | [′CAGTGATGCCCAGCCCCACATCCAGBGGAT |
| | C (p.Trp290Arg) | | | CAAGCACGTGGAAAAGAACGG′] |
| 1445 | NM_000141.4(FGFR2): c.799T > | 2263 | FGFR2 | [′CCAAGCCGGACTGCCGGCAAATGCCYCCAC |
| | C (p.Ser267Pro) | | | AGTGGTCGGAGGAGACGTAGA′] |
| 1446 | NM_000506.3(F2): c.1139T > C | 2147 | F2 | [′CCTGCTGCCCCTCCCAGGCAGGTGAYGCTTT |
| | (p.Met380Thr) | | | TCCGGAAGAGTCCCCAGGAG′] |
| 1447 | NM_000313.3(PROS1): | 5627 | PROS1 | [′TTCATTATTTTAAATAGATAATGTABCCAGT |
| | c.1501T > C (p.Ser501Pro) | | | GCTGAGGGTTGGCATGTAAA′] |
| 1448 | NM_002834.3(PTPN11): c.854T > | 5781 | PTPN11 | [′TGACCGTGGTCTCTTTTTCTTCTAGNTGATCA |
| | C (p.Phe285Ser) | | | TACCAGGGTTGTCCTACAC′] |
| 1449 | NM_002755.3(MAP2K1): | 5604 | MAP2K1 | [′CAGCAGCGAAAGCGCCTTGAGGCCTYTCTT |
| | c.158T > C (p.Phe53Ser) | | | ACCCAGAAGCAGAAGGTGGGA′] |
| 1450 | NM_001042465.1(PSAP): | 5660 | PSAP | [′GCTTTTGACAAAATGTGCTCGAAGCYGCCG |
| | c.1055T > C (p.Leu352Pro) | | | AAGTCCCTGTCGGAAGAGTGC′] |
| 1451 | NM_000207.2(INS): c.143T > C | −1 | — | [′TACCTAGTGTGCGGGGAACGAGGCTBCTTCT |
| | (p.Phe48Ser) | | | ACACACCCAAGACCCGCCGG′] |
| 1452 | NM_000311.3(PRNP): c.593T > C | 5621 | PRNP | [′ACCACAACCACCAAGGGGGAGAACTYCACC |
| | (p.Phe198Ser) | | | GAGACCGACGTTAAGATGATG′] |
| 1453 | NM_000371.3(TTR): c. 149T > C | 7276 | TTR | [′GGCAGTCCTGCCATCAATGTGGCCGBGCATG |
| | (p.Val50Ala) | | | TGTTCAGAAAGGCTGCTGAT′] |
| 1454 | NM_000371.3(TTR): c.224T > C | 7276 | TTR | [′AGGAAAACCAGTGAGTCTGGAGAGCYGCAT |
| | (p.Leu75Pro) | | | GGGCTCACAACTGAGGAGGAA′] |
| 1455 | NM_000371.3(TTR): c.88T > C | 7276 | TTR | [′CACCCAGGGCACCGGTGAATCCAAGYGTCC |
| | (p.Cys30Arg) | | | TCTGATGGTCAAAGTTCTAGA′] |
| 1456 | NM_000371.3(TTR): c.272T > C | 7276 | TTR | [′GAATTTGTAGAAGGGATATACAAAGYGGAA |
| | (p.Val91Ala) | | | ATAGACACCAAATCTTACTGG′] |
| 1457 | NM_000371.3(TTR): c.400T > C | 7276 | TTR | [′CACCATTGCCGCCCTGCTGAGCCCCYACTCC |
| | (p.Tyr134His) | | | TATTCCACCACGGCTGTCGT′] |
| 1458 | NM_000371.3(TTR): c.250T > C | 7276 | TTR | [′GCATGGGCTCACAACTGAGGAGGAAYTTGT |
| | (p.Phe84Leu) | | | AGAAGGGATATACAAAGTGGA′] |
| 1459 | NM_000371.3(TTR): c.157T > C | 7276 | TTR | [′TGCCATCAATGTGGCCGTGCATGTGHTCAGA |
| | (p.Phe53Leu) | | | AAGGCTGCTGATGACACCTG′] |
| 1460 | NM_000371.3(TTR): c.95T > C | 7276 | TTR | [′GGCACCGGTGAATCCAAGTGTCCTCYGATG |
| | (p.Leu32Pro) | | | GTCAAAGTTCTAGATGCTGTC′] |
| 1461 | NM_000371.3(TTR): c.191T > C | 7276 | TTR | [′GCTGCTGATGACACCTGGGAGCCATYTGCCT |
| | (p.Phe64Ser) | | | CTGGGTAAGTTGCCAAAGAA′] |
| 1462 | NM_000371.3(TTR): c.265T > C | 7276 | TTR | [′TGAGGAGGAATTTGTAGAAGGGATAYACAA |
| | (p.Tyr89His) | | | AGTGGAAATAGACACCAAATC′] |
| 1463 | NM_000217.2(KCNA1): | 3736 | KCNA1 | [′ATTGCCCTGCCCGTACCTGTCATTGYGTCCA |
| | c.1223T > C (p. Val408Ala) | | | ATTTCAACTATTTCTACCAC′] |
| 1464 | NM_000174.4(GP9): c.212T > C | 2815 | GP9 | [′CTTCAGTCCGTGCCCCCGGGAGCCTBTGACC |
| | (p.Phe71Ser) | | | ACCTGCCCCAGCTGCAGACC′] |
| 1465 | NM_000174.4(GP9): c.167T > C | 2815 | GP9 | [′GCCCTGCCGGCCCGCACCCGCCACCYTCTGC |
| | (p.Leu56Pro) | | | TGGCCAACAACAGCCTTCAG′] |
| 1466 | NM_000174.4(GP9): c.70T > C | 2815 | GP9 | [′GGCCACCAAGGACTGCCCCAGCCCAYGTAC |
| | (p.Cys24Arg) | | | CTGCCGCGCCCTGGAAACCAT′] |
| 1467 | NM_000174.4(GP9): c.20T > C | 2815 | GP9 | [′TGTCCCATGCCTGCCTGGGGAGCCCYGTTCC |
| | (p.Leu7Pro) | | | TGCTCTGGGCCACAGCAGAG′] |
| 1468 | NM_001001547.2(CD36): | 948 | CD36 | [′CGATTTTTAAACAGATGCAGCCTCABTTCCA |
| | c.760T > C (p.Phe254Leu) | | | CCTTTTGTTGAGAAAAGCCA′] |
| 1469 | NM_000212.2(ITGB3): c.2332T > | −1 | — | [′CAACCCACTGTATAAAGAGGCCACGYCTAC |
| | C (p.Ser778Pro) | | | CTTCACCAATATCACGTACCG′] |
| 1470 | NM_000212.2(ITGB3): c.176T > | 3690 | ITGB3 | [′GCTCCTGTCTTACAGGCCCTGCCTCYGGGCT |
| | C (p.Leu59Pro) | | | CACCTCGCTGTGACCTGAAG′] |
| 1471 | NM_000301.3(PLG): c.1771T > C | 5340 | PLG | [′AGGGGGGTGTGTGGCCCACCCACATYCCTG |
| | (p.Ser591Pro) | | | GCCCTGGCAAGTCAGTCTTAG′] |
| 1472 | NM_001122757.2(POU1F1): | 5449 | POU1F1 | [′CAAACTGAAAGCAATATTATCCAAAYGGCT |
| | c.655T > C (p.Trp219Arg) | | | GGAGGAAGCTGAGCAAGTAGG′] |
| 1473 | NM_000293.2(PHKB): c.2923T > | 5257 | PHKB | [′ACCAACCCTGTCAGATATGACCATGHATGA |
| | C (p.Tyr975His) | | | GATGAATTTCTCTCTCCTTGT′] |
| 1474 | NM_004577.3(PSPH): c.155T > C | 5723 | PSPH | [′CTCCCTCCTAGGACACGGCGAGCCAYGGGC |
| | (p.Met52Thr) | | | GGGGCAGTGCCTTTCAAAGCT′] |
| 1475 | NM_000175.3(GPI): c.1574T > C | 2821 | GPI | [′CTGGGAAAGCAGCTGGCTAAGAAAABAGAG |
| | (p.Ile525Thr) | | | CCTGAGCTTGATGGCAGTGCT′] |
| 1476 | NM_000175.3(GPI): c.1016T > C | 2821 | GPI | [′TTTGGGTGTGAGACACACGCCATGCYGCCCT |
| | (p.Leu339Pro) | | | ATGACCAGTACCTGCACCGC′] |
| 1477 | NM_000478.4(ALPL): c.1306T > | 249 | ALPL | [′ACGAGAGAATGTCTCCATGGTGGACYATGG |
| | C (p.Tyr436His) | | | TGAGACCTCCAGGACCCAGGG′] |
| 1478 | NM_000478.4(ALPL): c.979T > C | 249 | ALPL | [′GATCCTGCGGAAGAACCCCAAAGGCYTCTT |
| | (p.Phe327Leu) | | | CTTGCTGGTGGAAGGTAGGGA′] |
| 1479 | NM_018849.2(ABCB4): | 5244 | ABCB4 | [′GGAGTTCAATGATGTTCACTTTTCTYACCCT |
| | c.1207T > C (p.Tyr403His) | | | TCTCGAGCTAACGTCAAGGT′] |
| 1480 | NM_198965.1(PTHLH): c.179T > | 5744 | PTHLH | [′CAAGATTTACGGCGACGATTCTTCCYTCACC |
| | C (p.Leu60Pro) | | | ATCTGATCGCAGAAATCCAC′] |
| 1481 | NM_198965.1(PTHLH): c.131T > | 5744 | PTHLH | [′AGAGCTGTGTCTGAACATCAGCTCCYCCATG |
| | C (p.Leu44Pro) | | | ACAAGGGGAAGTCCATCCAA′] |
| 1482 | NM_000315.2(PTH): c.52T > C | 5741 | PTH | [′AGTTATGATTGTCATGTTGGCAATTYGTTTT |
| | (p.Cys18Arg) | | | CTTACAAAATCGGATGGGAA′] |
| 1483 | NM_000315.2(PTH): c.67T > C | 5741 | PTH | [′GTTGGCAATTTGTTTTCTTACAAAAYCGGAT |
| | (p.Ser23Pro) | | | GGGAAATCTGTTAAGTAAGT′] |
| 1484 | NM_003122.4(SPINK1): c.2T > C | 6690 | SPINK1 | [′ACCTCTGGACGCAGAACTTCAGCCAYGAAG |
| | (p.Met1Thr) | | | GTAACAGGCATCTTTCTTCTC′] |
| 1485 | NM_003122.4(SPINK1): c.41T > | 6690 | SPINK1 | [′ATCTTTCTTCTCAGTGCCTTGGCCCBGTTGA |
| | C (p.Leu14Pro) | | | GTCTATCTGGTAAGTGTTGC′] |
| 1486 | NM_006194.3(PAX9): c.62T > C | 5083 | PAX9 | [′GGAGTGTTCGTGAACGGGAGGCCGCYGCCC |
| | (p.Leu21Pro) | | | AACGCCATCCGGCTTCGCATC′] |
| 1487 | NM_005188.3(CBL): c.1111T > C | 867 | CBL | [′TTAATCAAAGGAACAATATGAATTAHACTG |
| | (p.Tyr371His) | | | TGAGATGGGCTCCACATTCCA′] |
| 1488 | NM_005247.2(FGF3): c.466T > C | 2248 | FGF3 | [′CAGCGCCGAGAGACTGTGGTACGTGYCTGT |
| | (p.Ser156Pro) | | | GAACGGCAAGGGCCGGCCCCG′] |
| 1489 | NM_005247.2(FGF3): c.17T > C | 2248 | FGF3 | [′GATGCCACGATGGGCCTAATCTGGCYGCTA |
| | (p.Leu6Pro) | | | CTGCTCAGCCTGCTGGAGCCC′] |
| 1490 | NM_000222.2(KIT): c.1676T > C | 3815 | KIT | [′CCCATGTATGAAGTACAGTGGAAGGNTGTT |
| | (p.Val559Ala) | | | GAGGAGATAAATGGAAACAAT′] |
| 1491 | NM_005249.4(FOXG1): c.643T > | 2290 | FOXG1 | [′CATCTACGAGTTCATCATGAAGAACYTCCCT |
| | C (p.Phe215Leu) | | | TACTACCGCGAGAACAAGCA′] |
| 1492 | NM_001127500.1(MET): | 4233 | MET | [′CAATTTCTGACCGAGGGAATCATCAYGAAA |
| | c.3446T > C (p.Met1149Thr) | | | GATTTTAGTCATCCCAATGTC′] |
| 1493 | NM_020975.4(RET): c.1858T > C | 5979 | RET | [′CTTCCCTGAGGAGGAGAAGTGCTTCNGCGA |
| | (p.Cys620Arg) | | | GCCCGAAGACATCCAGGGTGA′] |
| 1494 | NM_020975.4(RET): c.1900T > C | 5979 | RET | [′CACAGATCCACTGTGCGACGAGCTGNGCCG |
| | (p.Cys634Arg) | | | CACGGTGATCGCAGCCGCTGT′] |
| 1495 | NM_020975.4(RET): c.2753T > C | 5979 | RET | [′TAGGGTCGGATTCCAGTTAAATGGAYGGCA |
| | (p.Met918Thr) | | | ATTGAATCCCTTTTTGATCAT′] |
| 1496 | NM_020630.4(RET): c.1852T > C | 5979 | RET | [′CAACTGCTTCCCTGAGGAGGAGAAGNGCTT |
| | (p.Cys618Arg) | | | CTGCGAGCCCGAAGACATCCA′] |
| 1497 | NM_020630.4(RET): c.1825T > C | 5979 | RET | [′GGGGATTAAAGCTGGCTATGGCACCNGCAA |
| | (p.Cys609Arg) | | | CTGCTTCCCTGAGGAGGAGAA′] |
| 1498 | NM_013251.3(TAC3): c.269T > C | 6866 | TAC3 | [′ATGCATGACTTCTTTGTGGGACTTAYGGGCA |
| | (p.Met90Thr) | | | AGAGGAGCGTCCAGCCAGGT′] |
| 1499 | NM_006158.4(NEFL): c.281T > C | 4747 | NEFL | [′ATCCGCACGCAGGAGAAGGCGCAGCYCCAG |
| | (p.Leu94Pro) | | | GACCTCAATGACCGCTTCGCC′] |
| 1500 | NM_006177.3(NRL): c.479T > C | 4901 | NRL | [′TGCGGGCGCGACGAGGCGCTGCGGCYGAAG |
| | (p.Leu160Pro) | | | CAGAGGCGCCGCACGCTGAAG′] |
| 1501 | NM_000432.3(MYL2): c.52T > C | 4633 | MYL2 | [′AGCCGGGGGCGCCAACTCCAACGTGYTCTC |
| | (p.Phe18Leu) | | | CATGTTCGAACAGACCCAAAT′] |
| 1502 | NM_000257.3(MYH7): c.1046T > | 4625 | MYH7 | [′TTCACTTCAGAGGAGAAAAACTCCAYGTAT |
| | C (p.Met349Thr) | | | AAGCTGACAGGCGCCATCATG′] |
| 1503 | NM_000257.3(MYH7): c.1594T > | 4625 | MYH7 | [′GCTTCCTCAGCCCATGGGCATCATGYCCATC |
| | C (p.Ser532Pro) | | | CTGGAAGAGGAGTGCATGTT′] |
| 1504 | NM_000257.3(MYH7): c.5378T > | 4625 | MYH7 | [′AACATGGAACAGACCATTAAGGACCYGCAG |
| | C (p.Leu1793Pro) | | | CACCGGCTGGACGAAGCCGAG′] |
| 1505 | NM_001040113.1(MYH11): | 4629 | MYH11 | [′GAGGTGGAACATAAGAAGAAGAAGCYGGA |
| | c.3791T > C (p.Leul264Pro) | | | GGCGCAGGTGCAGGAGCTGCAG′] |
| 1506 | NM_000530.6(MPZ): c.404T > C | 4359 | MPZ | [′TGTGACGTCAAAAACCCTCCAGACAYAGTG |
| | (p.Ile135Thr) | | | GGCAAGACCTCTCAGGTCACG′] |
| 1507 | NM_000530.6(MPZ): c.341T > C | 4359 | MPZ | [′CGCTGGAAGGATGGCTCCATTGTCAYACAC |
| | (p.Ile114Thr) | | | AACCTAGACTACAGTGACAAT′] |
| 1508 | NM_016835.4(MAPT): c.1839T > | 4137 | MAPT | [′AGTCCAAGTGTGGCTCAAAGGATAAYATCA |
| | C (p.Asn613=) | | | AACACGTCCCGGGAGGCGGCA′] |
| 1509 | NM_198159.2(MITF): c.1051T > | 4286 | MITF | [′GAACAAGGGAACCATCTTAAAAGCAYCCGT |
| | C (p.Ser351Pro) | | | GGACTATATCCGAAAGTTGCA′] |
| 1510 | NM_198159.2(MITF): c.1195T > | 4286 | MITF | [′AATGCAGGCTCGAGCTCATGGACTTYCCCTT |
| | C (p.Ser399Pro) | | | ATTCCATCCACGGGTCTCTG′] |
| 1511 | NM_000426.3(LAMA2): | 3908 | LAMA2 | [′ACCAAGAATGAGTCCGGCATCATTCYTTTGG |
| | c.7691T > C (p.Leu2564Pro) | | | GAAGTGGAGGGACACCAGCA′] |
| 1512 | NM_000426.3(LAMA2): | 3908 | LAMA2 | [′ACAACCCTCTGTACCTGGAGGATCAYGTCA |
| | c.2584T > C (p.Cys862Arg) | | | GCCATGCCAATGCAATGACAA′] |
| 1513 | NM_002435.2(MPI): c.413T > C | 4351 | MPI | [′CCCGATGCCAACCACAAGCCAGAGAYGGCC |
| | (p.Met138Thr) | | | ATTGCCCTCACCCCCTTCCAG′] |
| 1514 | NM_000239.2(LYZ): c.221T > C | 4069 | LYZ | [′GGAGACAGAAGCACTGATTATGGGAYATTT |
| | (p.Ile74Thr) | | | CAGATCAATAGCCGCTACTGG′] |
| 1515 | NM_000239.2(LYZ): c.244T > C | 4069 | LYZ | [′GATATTTCAGATCAATAGCCGCTACHGGTGT |
| | (p.Trp82Arg) | | | AATGATGGCAAAACCCCAGG′] |
| 1516 | NM_000233.3(LHCGR): | −1 | — | [′CAATGTGGTGGCCTTCTTCATAATTYGTGCT |
| | c.1627T > C (p.Cys543Arg) | | | TGCTACATTAAAATTTATTT′] |
| 1517 | NM_000233.3(LHCGR): | −1 | — | [′AAACTTACAGTGCCTCGTTTTCTCAYGTGCA |
| | c.1193T > C (p.Met398Thr) | | | ATCTCTCCTTTGCAGACTTT′] |
| 1518 | NM_000233.3(LHCGR): c.391T > | −1 | — | [′TCCTGTCCCTAATCACAGGAGCATCYGTAAC |
| | C (p.Cys131Arg) | | | ACAGGCATCAGAAAGTTTCC′] |
| 1519 | NM_000233.3(LHCGR): | −1 | — | [′GACTTCCTTAGGGTCCTGATTTGGCYGATTA |
| | c.1103T > C (p.Leu368Pro) | | | ATATTCTAGCCATCATGGGA′] |
| 1520 | NM_000233.3(LHCGR): | −1 | — | [′TCTTCTCTAATTGCTATGTTGCCCCYTGTCGG |
| | c.1505T > C (p.Leu502Pro) | | | TGTCAGCAATTACATGAAG′] |
| 1521 | NM_170707.3(LMNA): c.1589T > | 4000 | LMNA | [′TGCGGGAACAGCCTGCGTACGGCTCYCATC |
| | C (p.Leu530Pro) | | | AACTCCACTGGGGAAGTAAGT′] |
| 1522 | NM_170707.3(LMNA): c.1139T > | 4000 | LMNA | [′GAGATCCACGCCTACCGCAAGCTCTYGGAG |
| | C (p.Leu380Ser) | | | GGCGAGGAGGAGAGGTGGGCT′] |
| 1523 | NM_002292.3(LAMB2): c.961T > | 3913 | LAMB2 | [′CAAACACAACACACGTGGCCTCAACYGCGA |
| | C (p.Cys321Arg) | | | GCAGTGTCAGGATTTCTATCG′] |
| 1524 | NM_000228.2(LAMB3): | 3914 | LAMB3 | [′AAATCCATAAGGTTAAGTTGGACCTRCAGA |
| | c.565 − 3T > C | | | GGGAAGGGAAAGAGAAGCGCT′] |
| 1525 | NM_000421.3(KRT10): c.482T > | −1 | — | [′CTGAATGACCGCCTGGCTTCCTACTYGGACA |
| | C (p.Leu161Ser) | | | AAGTTCGGGCTCTGGAAGAA′] |
| 1526 | NM_000421.3(KRT10): c.449T > | −1 | — | [′CTCTCTGGAAATGAAAAAGTAACCABGCAG |
| | C (p.Met150Thr) | | | AATCTGAATGACCGCCTGGCT′] |
| 1527 | NM_000422.2(KRT17): c.263T > | 3872 | KRT17 | [′CTGGCTGGAGGTGAGAAGGCCACCAHGCAG |
| | C (p.Met88Thr) | | | AACCTCAATGACCGCCTGGCC′] |
| 1528 | NM_000422.2(KRT17): c.284T > | 3872 | KRT17 | [′ACCATGCAGAACCTCAATGACCGCCHGGCC |
| | C (p.Leu95Pro) | | | TCCTACCTGGACAAGGTGCGT′] |
| 1529 | NM_000422.2(KRT17): c.296T > | 3872 | KRT17 | [′CTCAATGACCGCCTGGCCTCCTACCYGGACA |
| | C (p.Leu99Pro | | | AGGTGCGTGCCCTGGAGGAG′] |
| 1530 | NM_005557.3(KRT16): c.395T > | 3868 | KRT16 | [′CTCAATGACCGCCTGGCCTCCTACCYGGACA |
| | C (p.Leu132Pro) | | | AGGTGCGTGCTCTGGAGGAG′] |
| 1531 | NM_005557.3(KRT16): c.362T > | 3868 | KRT16 | [′CTGGTGGGCAGTGAGAAGGTGACCAHGCAG |
| | C (p.Met121Thr) | | | AACCTCAATGACCGCCTGGCC′] |
| 1532 | NM_000526.4(KRT14): | 3861 | KRT14 | [′ATGATTGGCAGCGTGGAGGAGCAGCYGGCC |
| | c.1151T> C (p.Leu384Pro) | | | CAGCTCCGCTGCGAGATGGAG′] |
| 1533 | NM_000526.4(KRT14): c.356T > | 3861 | KRT14 | [′CTGGTGGGCAGTGAGAAGGTGACCAYGCAG |
| | C (p.Met119Thr) | | | AACCTCAATGACCGCCTGGCC′] |
| 1534 | NM_000526.4(KRT14): | 3861 | KRT14 | [′GCGGCTGGAGCAGGAGATCGCCACCYACCG |
| | c.1243T > C (p.Tyr415His) | | | CCGCCTGCTGGAGGGCGAGGA′] |
| 1535 | NM_153490.2(KRT13): c.356T > | 3860 | KRT13 | [′CTCAACGACCGCCTGGCTTCCTACCYGGAGA |
| | C (p.Leu119Pro) | | | AGGTGCGCGCCCTGGAGGAG′] |
| 1536 | NM_002273.3(KRT8): c.160T > C | 3856 | KRT8 | [′CTTTCGCGGTGGCCTGGGCGGCGGCHATGGT |
| | (p.Tyr54His) | | | GGGGCCAGCGGCATGGGAGG′] |
| 1537 | NM_000424.3(KRT5): c.1388T > | 3852 | KRT5 | [′GAGCTCATGAACACCAAGCTGGCCCYGGAC |
| | C (p.Leu463Pro) | | | GTGGAGATCGCCACTTACCGC′] |
| 1538 | NM_000424.3(KRT5): c.980T > C | 3852 | KRT5 | [′TCTGACACCTCAGTGGTCCTCTCCAHGGACA |
| | (p.Met327Thr) | | | ACAACCGCAACCTGGACCTG′] |
| 1539 | NM_000424.3(KRT5): c.20T > C | 3852 | KRT5 | [′GCCACCATGTCTCGCCAGTCAAGTGYGTCCT |
| | (p.Val7Ala) | | | TCCGGAGCGGGGGCAGTCGT′] |
| 1540 | NM_000424.3(KRT5): c.541T > C | 3852 | KRT5 | [′CAAGACCCTCAACAATAAGTTTGCCYCCTTC |
| | (p.Ser181Pro) | | | ATCGACAAGGTGAGCTACGA′] |
| 1541 | NM_000208.2(INSR): c.779T > C | 3643 | INSR | [′TGCGTGGCCTGCCGCAACTTCTACCYGGACG |
| | (p.Leu260Pro) | | | GCAGGTGTGTGGAGACCTGC′] |
| 1542 | NM_000208.2(INSR): c.164T > C | 3643 | INSR | [′TTGCATGAGCTGGAGAATTGCTCTGYCATCG |
| | (p.Val55Ala) | | | AAGGACACTTGCAGATACTC′] |
| 1543 | NM_002198.2(IRF1): c.31T > C | 3659 | IRF1 | [′CATCACTCGGATGCGCATGAGACCCYGGCT |
| | (p.Trp11Arg) | | | AGAGATGCAGATTAATTCCAA′] |
| 1544 | NM_000213.3(ITGB4): c.467T > | 3691 | ITGB4 | [′AACCTCAAGAAGATGGGGCAGAACCYGGGT |
| | C (p.Leu156Pro) | | | ACGGCAGGGCCAGAGTGGAGG′] |
| 1545 | NM_000213.3(ITGB4): c.1684T > | 3691 | ITGB4 | [′CCGAGGACGCTGCTCCATGGGCCAGYGTGT |
| | C (p.Cys562Arg) | | | GTGTGAGCCTGGTTGGACAGG′] |
| 1546 | NM_000213.3(ITGB4): c.112T > | 3691 | ITGB4 | [′CTGCAAGAAGGCCCCAGTGAAGAGCYGCAC |
| | C (p.Cys38Arg) | | | GGAGTGTGTCCGTGTGGATAA′] |
| 1547 | NM_000454.4(SOD1): c.338T > C | 6647 | SOD1 | [′ATCTCACTCTCAGGAGACCATTGCAYCATTG |
| | (p.Ile113Thr) | | | GCCGCACACTGGTGGTAAGT′] |
| 1548 | NM_000454.4(SOD1): c.434T > C | 6647 | SOD1 | [′AAGACAGGAAACGCTGGAAGTCGTTYGGCT |
| | (p.Leu145Ser) | | | TGTGGTGTAATTGGGATCGCC′] |
| 1549 | NM_000454.4(SOD1): c.455T > C | 6647 | SOD1 | [′CGTTTGGCTTGTGGTGTAATTGGGAYCGCCC |
| | (p.Ile152Thr) | | | AATAAACATTCCCTTGGATG′] |
| 1550 | NM_001025107.2(ADAR): | 103 | ADAR | [′ACCAGACTTTCTTTTTGTAGGTTTCYCTACA |
| | c.1883T > C (p.Leu628Pro) | | | GTGAGTTAATGAAATACAAC′] |
| 1551 | NM_001025107.2(ADAR): | 103 | ADAR | [′TCCAAAAAGAACATTTTTCTTCTATYTAAGA |
| | c.2609T > C (p.Phe870Ser) | | | AGCTCTGCTCCTTCCGTTAC′] |
| 1552 | NM_002185.3(IL7R): c.197T > C | 3575 | IL7R | [′CCATATTTCAAATTCCAGATTGGTGRTGTTG |
| | (p.Ile66Thr) | | | ACATCTGGGTCCTCAAAAGC′] |
| 1553 | NM_033500.2(HK1): c.1550T > C | 3098 | HK1 | [′ACTGCAACAGAGAATGGTGACTTCTYGGCC |
| | (p.Leu517Ser) | | | CTGGATCTTGGAGGAACCAAT′] |
| 1554 | NM_000184.2(HBG2): | 3048 | HBG2 | [′CTTCCCCACACTATCTCAATGCAAAYATCTG |
| | c.−228T > C | | | TCTGAAACGGTCCCTGGCTA′] |
| 1555 | NM_000184.2(HBG2): c.125T > C | 3048 | HBG2 | [′GTTGTCTACCCATGGACCCAGAGGTYCTTTG |
| | (p.Phe42Ser) | | | ACAGCTTTGGCAACCTGTCC′] |
| 1556 | NM_000559.2(HBG1): | 3047 | HBG1 | [′TAGCAGTATCCTCTTGGGGGCCCCTYCCCCA |
| | c.−251T > C | | | CACTATCTCAATGCAAATAT′] |
| 1557 | NM_000519.3(HBD): c.−127T > C | 3045 | HBD | [′TCTCACAAACTAATGAAACCCTGCTYATCTT |
| | | | | AAACCAACCTGCTCACTGGA′] |
| 1558 | NM_000518.4(HBB): c.257T > C | 3043 | HBB | [′CACCTGGACAACCTCAAGGGCACCTYTGCC |
| | (p.Phe86Ser) | | | ACACTGAGTGAGCTGCACTGT′] |
| 1559 | NM_000518.4(HBB): c.128T > C | 3043 | HBB | [′GTCTACCCTTGGACCCAGAGGTTCTBTGAGT |
| | (p.Phe43Ser) | | | CCTTTGGGGATCTGTCCACT′] |
| 1560 | NM_000518.4(HBB): c.337T > C | 3043 | HBB | [′ACAGCTCCTGGGCAACGTGCTGGTCYGTGTG |
| | (p.Cys113Arg) | | | CTGGCCCATCACTTTGGCAA′] |
| 1561 | NM_000518.4(HBB): c.127T > C | 3043 | HBB | [′GGTCTACCCTTGGACCCAGAGGTTCBTTGAG |
| | (p.Phe43Leu) | | | TCCTTTGGGGATCTGTCCAC′] |
| 1562 | NM_000518.4(HBB): c.332T > C | 3043 | HBB | [′CTCCCACAGCTCCTGGGCAACGTGCYGGTCT |
| | (p.Leul11Pro) | | | GTGTGCTGGCCCATCACTTT′] |
| 1563 | NM_000518.4(HBB): | 3043 | HBB | [′GGTGGTGAGGCCCTGGGCAGGTTGGHATCA |
| | c.92 + 6T > C | | | AGGTTACAAGACAGGTTTAAG′] |
| 1564 | NM_000518.4(HBB): c.344T > C | 3043 | HBB | [′CTGGGCAACGTGCTGGTCTGTGTGCYGGCCC |
| | (p.Leu115Pro) | | | ATCACTTTGGCAAAGAATTC′] |
| 1565 | NM_000517.4(HBA2): c.427T > C | 3040 | HBA2 | [′CACCGTGCTGACCTCCAAATACCGTNAAGCT |
| | (p.Ter143Gln) | | | GGAGCCTCGGTAGCCGTTCC′] |
| 1566 | NM_000517.4(HBA2): c.2T > C | 3040 | HBA2 | [′CCACAGACTCAGAGAGAACCCACCAYGGTG |
| | (p.Met1Thr) | | | CTGTCTCCTGCCGACAAGACC′] |
| 1567 | NM_000517.4(HBA2): c.89T > C | 3040 | HBA2 | [′GCTGGCGAGTATGGTGCGGAGGCCCYGGAG |
| | (p.Leu30Pro) | | | AGGTGAGGCTCCCTCCCCTGC′] |
| 1568 | NM_006121.3(KRT1): c.482T > C | 3848 | KRT1 | [′CAAGAAGTCACTATCAACCAGAGCCYTCTTC |
| | (p.Leul61Pro) | | | AGCCCCTCAATGTGGAGATT′] |
| 1569 | NM_006121.3(KRT1): c.1436T > | 3848 | KRT1 | [′ACCAAGCTGGCCCTGGATCTGGAGAYTGCC |
| | C (p.Ile479Thr) | | | ACCTACAGGACCCTCCTGGAG′] |
| 1570 | NM_006121.3(KRT1): c.1424T > | 3848 | KRT1 | [′GAGCTGATGAACACCAAGCTGGCCCYGGAT |
| | C (p.Leu475Pro) | | | CTGGAGATTGCCACCTACAGG′] |
| 1571 | NM_001077488.3(GNAS): | 2778 | GNAS | [′AAAGTGCAGGACATCAAAAACAACCYGAAA |
| | c.299T > C (p.Leu100Pro) | | | GAGGCGATTGAAGTACGTGCT′] |
| 1572 | NM_000515.4(GH1): | 2688 | GH1 | [′AGGAAACACAACAGAAATCCGTGAGYGGAT |
| | c.291 + 6T > C | | | GCCTTCTCCCCAGGCGGGGAT′] |
| 1573 | NM_002087.3(GRN): c.2T > C | 2896 | GRN | [′TCCTTGGTACTTTGCAGGCAGACCAYGTGGA |
| | (p.Met1Thr) | | | CCCTGGTGAGCTGGGTGGCC′] |
| 1574 | NM_021957.3(GYS2): c.1447T > | 2998 | GYS2 | [′GGTGATTTTGCACCCAGAGTTTCTAYCCTCC |
| | C (p.Ser483Pro) | | | ACCAGTCCCTTACTACCCAT′] |
| 1575 | NM_001083112.2(GPD2): | 2820 | GPD2 | [′AGGTATAAGAAGAGATTTCATAAGTYTGAT |
| | c.1904T > C (p.Phe635Ser) | | | GCAGACCAGAAAGGCTTTATT′] |
| 1576 | NM_000340.1(SLC2A2): | 6514 | SLC2A2 | [′ATCTTCATGTCAGTGGGACTTGTGCBGCTGG |
| | c.1166T > C (p.Leu389Pro) | | | TAAGTTTGGTGCCTGCACTG′] |
| 1577 | NM_005271.3(GLUD1): | 2746 | GLUD1 | [′GTGTTCTTTTCCCTAATAGGGTGCAYCTGAG |
| | c.1501T > C (p.Ser501Pro) | | | AAAGACATCGTGCACTCTGG′] |
| 1578 | NM_000162.3(GCK): c.391T > C | 2645 | GCK | [′CTTCGACTACATCTCTGAGTGCATCYCCGAC |
| | (p.Ser131Pro) | | | TTCCTGGACAAGCATCAGAT′] |
| 1579 | NM_001018077.1(NR3C1): | 2908 | NR3C1 | [′CTCAACATGTTAGGAGGGCGGCAAGYGATT |
| | c.1712T > C (p.Val571Ala) | | | GCAGCAGTGAAATGGGCAAAG′] |
| 1580 | NM_001018077.1(NR3C1): | 2908 | NR3C1 | [′TCAAATGGAAATATCAAAAAACTTCYGTTTC |
| | c.2318T > C (p.Leu773Pro) | | | ATCAAAAGTGACTGCCTTAA′] |
| 1581 | NM_001018077.1(NR3C1): | 2908 | NR3C1 | [′GGTTGAAAATCTCCTTAACTATTGCYTCCAA |
| | c.2209T > C (p.Phe737Leu) | | | ACATTTTTGGATAAGACCAT′] |
| 1582 | NM_002055.4(GFAP): c.1055T > | 2670 | GFAP | [′CGCCACTTGCAGGAGTACCAGGACCYGCTC |
| | C (p.Leu352Pro) | | | AATGTCAAGCTGGCCCTGGAC′] |
| 1583 | NM_002353.2(TACSTD2): | 4070 | TACSTD2 | [′CGGCTCTTCCGCGAGCGCTATCGGCYGCACC |
| | c.557T > C (p.Leu186Pro) | | | CCAAGTTCGTGGCGGCCGTG′] |
| 1584 | NM_000821.6(GGCX): c.896T > | 2677 | GGCX | [′TTTCTCCCTGCTTTCCTAGGTATGTYCTCCTA |
| | C (p.Phe299Ser) | | | CGTCATGCTGGCCAGCAGC′] |
| 1585 | NM_000145.3(FSHR): c.479T > C | 2492 | FSHR | [′CAAGATAACATAAACATCCACACAAYTGAA |
| | (p.Ile160Thr) | | | AGAAATTCTTTCGTGGGGCTG′] |
| 1586 | NM_000145.3(FSHR): c.1634T > | 2492 | FSHR | [′CTCAATGTCCTGGCCTTTGTGGTCAYCTGTG |
| | C (p.Ile545Thr) | | | GCTGCTATATCCACATCTAC′] |
| 1587 | NM_182925.4(FLT4): c.3131T > C | 2324 | FLT4 | [′AGAGACCTGGCTGCTCGGAACATTCYGCTGT |
| | (p.Leu1044Pro) | | | CGGAAAGCGACGTGGTGAAG′] |
| 1588 | NM_182925.4(FLT4): c.3257T > C | 2324 | FLT4 | [′CTGAAGTGGATGGCCCCTGAAAGCAYCTTC |
| | (p.Ile1086Thr) | | | GACAAGGTGTACACCACGCAG′] |
| 1589 | NM_023110.2(FGFR1): c.899T > | 2260 | FGFR1 | [′CACATCGAGGTGAATGGGAGCAAGAYTGGC |
| | C (p.Ile300Thr) | | | CCAGACAACCTGCCTTATGTC′] |
| 1590 | NM_023110.2(FGFR1): | 2260 | FGFR1 | [′CCTGTACCTGGAGATCATCATCTATYGCACA |
| | c.1141T > C (p.Cys381Arg) | | | GGGGCCTTCCTCATCTCCTG′] |
| 1591 | NM_000138.4(FBN1): c.4987T > | 2200 | FBN1 | [′TCCTGGAATCTGTGGTCCAGGGACAYGTTAC |
| | C (p.Cys1663Arg) | | | AACACCGTTGGCAACTACAC′] |
| 1592 | NM_000138.4(FBN1): c.3220T > | 2200 | FBN1 | [′CTGTTTTTGTGCAGACATTGACGAAYGCCGC |
| | C (p.Cys1074Arg) | | | ATATCTCCTGACCTCTGTGG′] |
| 1593 | NM_000138.4(FBN1): c.3793T > | 2200 | FBN1 | [′TATCCCTGGAGAGTACAGGTGCTTGYGTTAT |
| | C (p.Cys1265Arg) | | | GATGGATTCATGGCATCTGA′] |
| 1594 | NM_000043.4(FAS): c.532T > C | 355 | FAS | [′ATCCAGATCTAACTTGGGGTGGCTTYGTCTT |
| | (p.Cys178Arg) | | | CTTCTTTTGCCAATTCCACT′] |
| 1595 | NM_000043.4(FAS): | 355 | FAS | [′CATGAATCTCCAACTTTAAATCCTGHAGGTA |
| | c.651 + 2T > C | | | TTGAAATAGGTATCAGCTTT′] |
| 1596 | NM_000129.3(F13A1): c.728T > C | 2162 | F13A1 | [′ATCCTGGACACTTGCCTGTATGTGAYGGACA |
| | (p.Met243Thr) | | | GAGCACAAATGGACCTCTCT′] |
| 1597 | NM_000186.3(CFH): c.1606T > C | 3075 | CFH | [′GCTGAATGACACATTGGACTATGAAYGCCA |
| | (p.Cys536Arg) | | | TGATGGTTATGAAAGCAATAC′] |
| 1598 | NM_000123.3(ERCC5): | −1 | — | [′AAGTTAATAAATTTGGCTTATTTGCYTGGAA |
| | c.2573T > C (p.Leu858Pro) | | | GTGATTATACCGAAGGAATA′] |
| 1599 | NM_000122.1(ERCC3): c.296T > | 2071 | ERCC3 | [′CCAGTTTACAAATATGCCCAAGACTYCTTGG |
| | C (p.Phe99Ser) | | | TGGCTATTGCAGAGCCAGTG′] |
| 1600 | NM_001113755.2(TYMP): | 1890 | TYMP | [′CTGGGTCGCTGCGTGGGCCACGCCCYGGAG |
| | c.854T > C (p.Leu285Pro) | | | GTGGAGGAGGCGCTGCTCTGC′] |
| 1601 | NM_000118.3(ENG): c.2T > C | 2022 | ENG | [′CGCACAGGCCCCCACGTGGACAGCAYGGAC |
| | (p.Met1Thr) | | | CGCGGCACGCTCCCTCTGGCT′] |
| 1602 | NM_203342.2(EPB41): c.2T > C | 2035 | EPB41 | [′AAACCAATCAGAAAACACAGGAACABGCAC |
| | (p.Met1Thr) | | | TGCAAGGTTTCTTTGTTGGAT′] |
| 1603 | NM_001972.2(ELANE): c.211T > | 1991 | ELANE | [′CAACTTCGTCATGTCGGCCGCGCACYGCGTG |
| | C (p.Cys71Arg) | | | GCGAATGTGTGAGTAGCCGG′] |
| 1604 | NM_000400.3(ERCC2): | 2068 | ERCC2 | [′ACCATGGCAACCTTCACCATGACGCYGGCA |
| | c.1454T > C (p.Leu485Pro) | | | CGGGTCTGCCTCTGCCCTATG′] |
| 1605 | NM_000281.3(PCBD1): c.244T > | 5092 | PCBD1 | [′CCACATCACGCTGAGCACCCATGAGYGTGC |
| | C (p.Cys82Arg) | | | CGGCCTTTCAGAACGGGACAT′] |
| 1606 | NM_001927.3(DES): c.1034T > C | 1674 | DES | [′CCCCCTCTCCTGCAGAACGATTCCCYGATGA |
| | (p.Leu345Pro) | | | GGCAGATGCGGGAATTGGAG′] |
| 1607 | NM_001927.3(DES): c.1154T > C | 1674 | DES | [′CTCAAGGATGAGATGGCCCGCCATCYGCGC |
| | (p.Leu385Pro) | | | GAGTACCAGGACCTGCTCAAC′] |
| 1608 | NM_000498.3(CYP11B2): | −1 | — | [′GTTCTGAAGCACCAAGTCTGAGCTCRCCACT |
| | c.1157T > C (p.Val386Ala) | | | CGCTCCAAAAACAGACCCAC′] |
| 1609 | NM_000498.3(CYP11B2): | −1 | — | [′CGGCGCCTGGCAGAGGCAGAGATGCYGCTG |
| | c.1382T > C (p.Leu461Pro) | | | CTGCTGCACCACGTAAGCAGG′] |
| 1610 | NM_001886.2(CRYBA4): | 1413 | CRYBA4 | [′TACCCCGCCGAGAGGCTCACCTCCTYCCGGC |
| | c.281T > C (p.Phe94Ser) | | | CTGCGGCCTGTGCTGTAAGT′] |
| 1611 | NM_001886.2(CRYBA4): | 1413 | CRYBA4 | [′GGCTTCCAAGGGCAGCAGTACATTCYGGAA |
| | c.206T > C (p.Leu69Pro) | | | CGAGGCGAATATCCAAGCTGG′] |
| 1612 | NM_000762.5(CYP2A6): | −1 | — | [′GCAACCCCAGCTCTATGAGATGTTCYCTTCG |
| | c.670T > C (p.Ser224Pro) | | | GTGATGAAACACCTGCCAGG′] |
| 1613 | NM_000165.4(GJA1): c.52T > C | 2697 | GJA1 | [′ACTCCTTGACAAGGTTCAAGCCTACYCAACT |
| | (p.Ser18Pro) | | | GCTGGAGGGAAGGTGTGGCT′] |
| 1614 | NM_000165.4(GJA1): c.32T > C | 2697 | GJA1 | [′GACTGGAGCGCCTTAGGCAAACTCCYTGAC |
| | (p.Leu11Pro) | | | AAGGTTCAAGCCTACTCAACT′] |
| 1615 | NM_004004.5(GJB2): c.101T > C | 2706 | GJB2 | [′CTCCTTTGCAGCCACAACGAGGATCVTAATG |
| | (p.Met34Thr) | | | CGAAAAATGAAGAGGACGGT′] |
| 1616 | NM_004004.5(GJB2): c.229T > C | 2706 | GJB2 | [′CTTCCCCATCTCCCACATCCGGCTAYGGGCC |
| | (p.Trp77Arg) | | | CTGCAGCTGATCTTCGTGTC′] |
| 1617 | NM_004004.5(GJB2): c.269T > C | 2706 | GJB2 | [′ATCTTCGTGTCCACGCCAGCGCTCCYAGTGG |
| | (p.Leu90Pro) | | | CCATGCACGTGGCCTACCGG′] |
| 1618 | NM_005215.3(DCC): c.503T > C | 1630 | DCC | [′AAGTGTGAAGTCATTGGGGAGCCCAYGCCA |
| | (p.Met168Thr) | | | ACAATCCACTGGCAGAAGAAC′] |
| 1619 | NM_001849.3(COL6A2): | 1292 | COL6A2 | [′GAGTGAAAACCTCTACTCCATCGCCYGCGA |
| | c.2329T > C (p.Cys777Arg) | | | CAAGCCACAGCAGGTGCGCAA′] |
| 1620 | NM_000090.3(COL3A1): c.582 + | 1281 | COL3A1 | [′CTGGTCATCCTGGTTCCCCTGTAAGHATAGC |
| | 6T > C | | | CATTGGTGGTGTTTTCTTCC′] |
| 1621 | NM_000089.3(COL1A2): c.279 + | 1278 | COL1A2 | [′GTTGGACTTGGCCCTGGACCAATGGYATGCT |
| | 2T > C | | | TATCTGTTTATCTTAGCCAA′] |
| 1622 | NM_000089.3(COL1A2): | 1278 | COL1A2 | [′TTGCAAGGTCTGCCTGGTATCGCTGYAAGTA |
| | c.3105 + 2T > C | | | AACTGTAGCCATCTCGCACA′] |
| 1623 | NM_000088.3(COL1A1): | 1277 | COL1A1 | [′TTCGACGTTGGCCCTGTCTGCTTCCYGTAAA |
| | c.4391T > C (p.Leu1464Pro) | | | CTCCCTCCATCCCAACCTGG′] |
| 1624 | NM_000493.3(COL10A1): | −1 | — | [′AAAGGGACTCATGTTTGGGTAGGCCYGTAT |
| | c.1841T > C (p.Leu614Pro) | | | AAGAATGGCACCCCTGTAATG′] |
| 1625 | NM_000493.3(COL10A1): | −1 | — | [′TGACCCAAGGACTGGAATCTTTACTYGTCAG |
| | c.1771T > C (p.Cys591Arg) | | | ATACCAGGAATATACTATTT′] |
| 1626 | NM_000493.3(COL10A1): | −1 | — | [′CGATCTCACAGAAAATGACCAGGTGYGGCT |
| | 1c.195T > C (p.Trp651Arg) | | | CCAGCTTCCCAATGCCGAGTC′] |
| 1627 | NM_000493.3(COL10A1): | −1 | — | [′ATACTCCTCTGAGTATGTCCACTCCYCTTTCT |
| | c.2011T > C (p.Ser671Pro) | | | CAGGATTCCTAGTGGCTCC′] |
| 1628 | NM_000493.3(COL10A1): | −1 | — | [′TCAGATACCAGGAATATACTATTTTYCATAC |
| | c.1798T > C (p.Ser600Pro) | | | CACGTGCATGTGAAAGGGAC′] |
| 1629 | NM_020549.4(CHAT): c.629T > C | 1103 | CHAT | [′ATGTATCTCAACAACCGCCTGGCCCYGCCTG |
| | (p.Leu210Pro) | | | TCAACTCCAGCCCTGCCGTG′] |
| 1630 | NM_020549.4(CHAT): c.914T > C | 1103 | CHAT | [′ATCATGCCGGAGCCTGAGCACGTCABCGTA |
| | (p.Ile305Thr) | | | GCCTGCTGCAATCAGGTAAGC′] |
| 1631 | NM_020549.4(CHAT): c.1007T > | 1103 | CHAT | [′GATCTGTTCACTCAGTTGAGAAAGAYAGTC |
| | C (p.Ile336Thr) | | | AAAATGGCTTCCAACGAGGAC′] |
| 1632 | NM_001822.5(CHN1): c.427T > C | 1123 | CHN1 | [′TGCCAAGATGACGATAAACCCAATTYATGA |
| | (p.Tyr143His) | | | GCACGTAGGATACACAACCTT′] |
| 1633 | NM_001904.3(CTNNB1): | 1499 | CTNNB1 | [′TTCTGGTGCCACTACCACAGCTCCTBCTCTG |
| | c.133T> C (p.Ser45Pro) | | | AGTGGTAAAGGCAATCCTGA′] |
| 1634 | NM_004056.4(CA8): c.298T > C | 767 | CA8 | [′ACATATATTTTCTTTTTCAGTTCTTYCGGGAG |
| | (p.Ser100Pro) | | | GACCATTGCCTCAAGGGCA′] |
| 1635 | NM_016124.4(RHD): c.329T > C | 6007 | RHD | [′CCTTCTGGGAAGGTGGTCATCACACYGTTCA |
| | (p.Leu110Pro) | | | GGTATTGGGATGGTGGCTGG′] |
| 1636 | NM_015865.6(SLC14A1): | 6563 | SLC14A1 | [′CTTTGGACTCTGGGGTTTCAACAGCYCTCTG |
| | c.871T > C (p.Ser291Pro) | | | GCCTGCATTGCAATGGGAGG′] |
| 1637 | NM_001250.5(CD40): c.247T > C | 958 | CD40 | [′GACACACTGCCACCAGCACAAATACYGCGA |
| | (p.Cys83Arg) | | | CCCCAGTGCGTGCGCTGTTGG′] |
| 1638 | NM_000342.3(SLC4A1): | 6521 | SLC4A1 | [′AGTGTGCGGCTCCCCCACAGGCCTGYCCATC |
| | c.2317T > C (p.Ser773Pro) | | | CTCATGGAGCCCATCCTGTC′] |
| 1639 | NM_001681.3(ATP2A2): | 488 | ATP2A2 | [′GGGTAGTGGCAGCGACACACTGCGAYGCCT |
| | c.1678T > C (p.Cys560Arg) | | | GGCCCTGGCCACTCATGACAA′] |
| 1640 | NM_001082971.1(DDC): | 1644 | DDC | [′TCCCCACAAATGGCTATTGGTGAATYTTGAC |
| | c.925T > C (p.Phe309Leu) | | | TGTTCTGCCATGTGGTAAGT′] |
| 1641 | NM_031226.2(CYP19A1): | −1 | — | [′ATACAAAAAGTATGAGAAGTCTGTGYAAGT |
| | c.743 + 2T > C | | | AATACAACTTTGGAAGATTTA′] |
| 1642 | NM_000486.5(AQP2): c.646T > C | −1 | — | [′ACCCCTGGTGGGCGCCATCCTGGGCYCCCTC |
| | (p.Ser216Pro) | | | CTCTACAACTACGTGCTGTT′] |
| 1643 | NM_000041.3(APOE): c.388T > C | 348 | APOE | [′GCTGGGCGCGGACATGGAGGACGTGYGCGG |
| | (p.Cys130Arg) | | | CCGCCTGGTGCAGTACCGCGG′] |
| 1644 | NM_000041.3(APOE): c.137T > C | 348 | APOE | [′TGGCAGAGCGGCCAGCGCTGGGAACYGGCA |
| | (p.Leu46Pro) | | | CTGGGTCGCTTTTGGGATTAC′] |
| 1645 | NM_000039.1(APOA1): c.220T > | −1 | — | [′CTCCAGCCTAAAGCTCCTTGACAACYGGGA |
| | C (p. Trp74Arg) | | | CAGCGTGACCTCCACCTTCAG′] |
| 1646 | NM_000039.1(APOA1): c.341T > | −1 | — | [′CTGAGGCAGGAGATGAGCAAGGATCYGGAG |
| | C (p.Leul14Pro) | | | GAGGTGAAGGCCAAGGTGCAG′] |
| 1647 | NM_000039.1(APOA1): c.593T > | 335 | APOA1 | [′TACAGCGACGAGCTGCGCCAGCGCTYGGCC |
| | C (p.Leu198Ser) | | | GCGCGCCTTGAGGCTCTCAAG′] |
| 1648 | NM_000416.2(IFNGR1): | 3459 | IFNGR1 | [′AATATTTCTCATCATTATTGTAATAYTTCTG |
| | c.260T > C (p.Ile87Thr) | | | ATCATGTTGGTGATCCATCA′] |
| 1649 | NM_000488.3(SERPINC1): | 462 | SERPINC1 | [′TGTCGATCTGTTCAGCCCTGAAAAGYCCAAA |
| | c.1141T > C (p.Ser381Pro) | | | CTCCCAGGTTTGTCTAGGAA′] |
| 1650 | NM_000488.3(SERPINC1): | 462 | SERPINC1 | [′GTTTGACACCATATCTGAGAAAACAYCTGAT |
| | c.442T > C (p.Ser148Pro) | | | CAGATCCACTTCTTCTTTGC′] |
| 1651 | NM_000488.3(SERPINC1): | 462 | SERPINC1 | [′AAGGTTTATCTTTTGTCCTTGCTGCYCATTG |
| | Tc.68 > C (p.Leu23Pro) | | | GCTTCTGGGACTGCGTGACC′] |
| 1652 | NM_000488.3(SERPINC1): | 462 | SERPINC1 | [′CAGAGCGGCCATCAACAAATGGGTGYCCAA |
| | Tc.667 > C (p.Ser223Pro) | | | TAAGACCGAAGGCCGAATCAC′] |
| 1653 | NM_000488.3(SERPINC1): | 462 | SERPINC1 | [′TTTTGCTATGACCAAGCTGGGTGCCYGTAAT |
| | c.379T > C (p.Cys127Arg) | | | GACACCCTCCAGCAACTGAT′] |
| 1654 | NM_001085.4(SERPINA3): | 12 | SERPINA3 | [′GATAAGAATGTCATCTTCTCCCCACYGAGCA |
| | 3c.23T > C (p.Leu78Pro) | | | TCTCCACCGCCTTGGCCTTC′] |
| 1655 | NM_000341.3(SLC3A1): | 6519 | SLC3A1 | [′AATCAGTATGTCAACGTGATGAACAHGCTTC |
| | c.1400T > C (p.Met467Thr) | | | TTTTCACACTCCCTGGAACT′] |
| 1656 | NM_000341.3(SLC3A1): | −1 | — | [′AATCGAGCATGCTATTCCAGTGTACYGAAC |
| | c.2033T > C (p.Leu678Pro) | | | ATACTGTATACCTCGTGTTAG′] |
| 1657 | NM_000021.3(PSEN1): c.749T > | 5663 | PSEN1 | [′CCTGAATGGACTGCGTGGCTCATCTYGGCTG |
| | C (p.Leu250Ser) | | | TGATTTCAGTATATGGTAAA′] |
| 1658 | NM_000021.3(PSEN1): c.338T > | 5663 | PSEN1 | [′TTTTATACCCGGAAGGATGGGCAGCYGTAC |
| | C (p.Leu113Pro) | | | GTATGAGTTTTGTTTTATTAT′] |
| 1659 | NM_000021.3(PSEN1): c.497T > | 5663 | PSEN1 | [′CTTTTCTAGGTCATCCATGCCTGGCBTATTAT |
| | C (p.Leu166Pro) | | | ATCATCTCTATTGTTGCTG′] |
| 1660 | NM_000021.3(PSEN1): c.254T > | 5663 | PSEN1 | [′TATGGCGCCAAGCATGTGATCATGCYCTTTG |
| | C (p.Leu85Pro) | | | TCCCTGTGACTCTCTGCATG′] |
| 1661 | NM_001151.3(SLC25A4): | 291 | SLC25A4 | [′GCCTTCAAGGACAAGTACAAGCAGCYCTTC |
| | c.293T > C (p.Leu98Pro) | | | TTAGGGGGTGTGGATCGGCAT′] |
| 1662 | NM_001100.3(ACTA1): c.287T > | 58 | ACTA1 | [′TGGCACCACACCTTCTACAACGAGCYTCGCG |
| | C (p.Leu96Pro) | | | TGGCTCCCGAGGAGCACCCC′] |
| 1663 | NM_001100.3(ACTA1): c.668T > | 58 | ACTA1 | [′AAGGAGAAGCTGTGCTACGTGGCCCYGGAC |
| | C (p.Leu223Pro) | | | TTCGAGAACGAGATGGCGACG′] |
| 1664 | NM_000485.2(APRT): c.407T > C | 353 | APRT | [′CTCACCCTCCCATCCCCAGGAACCAYGAAC |
| | (p.Met136Thr) | | | GCTGCCTGTGAGCTGCTGGGC′] |
| 1665 | NM_000485.2(APRT): c.329T > C | 353 | APRT | [′CACCACTTCCCACTCCAGGCTGAGCYGGAG |
| | (p.Leu110Pro) | | | ATTCAGAAAGACGCCCTGGAG′] |
| 1666 | NM_001614.3(ACTG1): | 71 | ACTG1 | [′TACGACGAGTCGGGCCCCTCCATCGYCCACC |
| | c.1109T > C (p.Val370Ala) | | | GCAAATGCTTCTAAACGGAC′] |
| 1667 | NM_000751.2(CHRND): | 1144 | CHRND | [′CCGGCTGAAGTGGAATGCTGAAGAAYTTGG |
| | c.283T > C (p.Phe95Leu) | | | AAACATCAGTGTCCTGCGCCT′] |
| 1668 | NM_000751.2(CHRND): | 1144 | CHRND | [′GCCCTGGCCCTCACACTCTCCAACCYCATCT |
| | c.188T > C (p.Leu63Pro) | | | CCCTGGTGAGAGGCCCTCCG′] |
| 1669 | NM_001039523.2(CHRNA1): | 1134 | CHRNA1 | [′CTCTGTCTTACTGTCTTTGACTGTGYTCCTTC |
| | c.901T > C (p.Phe301Leu) | | | TGGTCATCGTGGAGCTGAT′] |
| 1670 | NM_001004127.2(ALG11): | 440138 | ALG11 | [′GGAGAAAGAGTTTTATGGTGTGCTTYAAGA |
| | c.257T > C (p.Leu86Ser) | | | GCCCTGCAGAAAAAGTAGGTA′] |
| 1671- | NM_002036.3(ACKR1): | 2532 | ACKR1 | [′AGAGGGAGCTAGGAGGCTAGCATAGRAAGG |
| 1674 | c.−67T > C | | | AGARAAGGAAAAAGACTTTGA′, |
| | | | | ′TATAAAGACTGAGGGGCAAACAGCASGGGA |
| | | | | AATGAGGGGCATAGGGATAAG′, |
| | | | | ′CGCAGACAGAAGGGCTGGGACGGCTRTCAG |
| | | | | CGCCTGTGCTTCCAAGRTAAG′, |
| | | | | ′ATCAGGAAGCCTTACCCCACGCCCAYTGCCT |
| | | | | GCACAAGCCTCAGGCCTATG′] |
| 1675 | NM_014053.3(FLVCR1): | 28982 | FLVCR1 | [′CAACTGCCTGGGTGCCTGGATCAAGYGCGG |
| | c.574T > C (p.Cys192Arg) | | | CAGTGTGCAGCAGCATCTCTT′] |
| 1676 | NM_004268.4(MED17): | 9440 | MED17 | [′TGTCCGGAGGACCACCTTTATGTCCYAGAGC |
| | c.1112T> C (p.Leu371Pro) | | | ATAATTTGCATCTACTGATT′] |
| 1677 | NM_024411.4(PDYN): c.632T > C | 5173 | PDYN | [′CTGTACAAACGCTATGGGGGCTTCTYGCGGC |
| | (p.Leu211Ser) | | | GCATTCGTCCCAAGCTCAAG′] |
| 1678 | NM_000018.3(ACADVL): | 37 | ACADVL | [′GTGAAGGAGAAGATCACAGCTTTTGYGGTG |
| | c.848T > C (p. Val283Ala) | | | GAGAGGGGCTTCGGGGGCATT′] |
| 1679 | NM_000021.3(PSEN1): c.1175T > | 5663 | PSEN1 | [′GGAGATTTCATTTTCTACAGTGTTCYGGTTG |
| | C (p.Leu392Pro) | | | GTAAAGCCTCAGCAACAGCC′] |
| 1680 | NM_000083.2(CLCN1): c.857T > | 1180 | CLCN1 | [′CCACACTTCTGTGCCCCTGCAGGAGYGCTAT |
| | C (p.Val286Ala) | | | TTAGCATCGAGGTCACCTCC′] |
| 1681 | NM_000083.2(CLCN1): c.920T > | 1180 | CLCN1 | [′GTTCGGAACTACTGGAGAGGATTCTYTGCA |
| | C (p.Phe307Ser) | | | GCCACGTTCAGCGCCTTTGTG′] |
| 1682 | NM_000157.3(GBA): c.703T > C | 2629 | GBA | [′CAATGGAGCGGTGAATGGGAAGGGGYCACT |
| | (p.Ser235Pro) | | | CAAGGGACAGCCCGGAGACAT′] |
| 1683 | NM_000166.5(GJB1): c.145T > C | 2705 | GJB1 | [′AGAGAGTGTGTGGGGTGATGAGAAAYCTTC |
| | (p.Ser49Pro) | | | CTTCATCTGCAACACACTCCA′] |
| 1684- | NM_000186.3(CFH): c.3590T > C | 3075 | CFH | [′ATATCCCCGTTTACACACAAATTCARCTGAT |
| 1686 | (p.Val1197Ala) | | | TCACCTGTTCTCGAATAAAG′, |
| | | | | ′GAACTTATCATTGCTGCTTTTGATTaagatat |
| | | | | tggtcaaagagtacaaact′, |
| | | | | ′ttcagctataatatgaataagaacttggaatc |
| | | | | aaacatagagatctagtga′] |
| 1687 | NM_000195.4(HPS1): c.2003T > | 3257 | HPS | [′GGGGATGACAGACAGGTGCAGGGCCRGCAG |
| | C (p.Leu668Pro) | | | CTCGTAGCACCTGACAGCCTC′] |
| 1688 | NM_000195.4(HPS1): c.716T > C | 3257 | HPST | [′GCTGGGGTAGAGGTCCTGAACCAGGRGGAT |
| | (p.Leu239Pro) | | | GAGGGCAAGCAGGTCGGCCGG′] |
| 1689 | NM_000334.4(SCN4A): | 6329 | SCN4A | [′CCTCTTCAACATCGGCCTCCTCCTCYTCCTG |
| | c.4468T > C (p.Phe1490Leu) | | | GTCATGTTCATCTACTCCAT′] |
| 1690 | NM_000352.4(ABCC8): c.257T > | 6833 | ABCC8 | [′ACCTTCATGCTGCTCTTCGTCCTGGBGTGTG |
| | C (p.Val86Ala) | | | AGATTGCAGAGGGCATCCTG′] |
| 1691 | NM_000352.4(ABCC8): c.404T > | 6833 | ABCC8 | [′ATCGAGACTTCCAACTTCCCCAAGCYGCTAA |
| | C (p.Leu135Pro) | | | TTGGTAGGTGAGGTGTAGGA′] |
| 1692 | NM_000352.4(ABCC8): c.674T > | 6833 | ABCC8 | [′TTCCTGCAGCCCTTCGTGAATCTGCYGTCCA |
| | C (p.Leu225Pro) | | | AAGGCACCTACTGGTGGATG′] |
| 1693 | NM_000430.3(PAFAH1B1): | 5048 | PAFAH1B1 | [′ATTACTTCATAATATATTGCTGTTAYGTGTTT |
| | c.569 − 10T > C | | | TAGGCCATGACCACAATGT′] |
| 1694 | NM_000525.3(KCNJ11): | 3767 | KCNJ11 | [′CCGTGCCCGCCAGCGGAGGGCCCGCBTTGT |
| | c.103T > C (p.Phe35Leu) | | | GTCCAAGAAAGGCAACTGCAA′] |
| 1695 | NM_000525.3(KCNJ11): | 3767 | KCNJ11 | [′GTGGGTGGCAACAGCATCTTCCTGGYGGCC |
| | c.755T > C (p. Val252Ala) | | | CCGCTGATCATCTACCATGTC′] |
| 1696 | NM_000528.3(MAN2B1): | 4125 | MAN2B1 | [′GGCAGCAGCCTGAGAGATGGCTCGCYGGAG |
| | c.2426T > C (p.Leu809Pro) | | | CTCATGGTGAGTGGGTCAGAG′] |
| 1697 | NM_001034850.2(FAM134B): | 54463 | FAM134B | [′GACTTTTCAGCTCTTTGTCCTAAGGYATTTTT |
| | c.873 + 2T > C | | | TGTTTAGTTTTCAATTTGT′] |
| 1698 | NM_001457.3(FLNB): c.4804T > | 2317 | FLNB | [′CTACGGGGGTGACGACATCCCACTTYCTCCT |
| | C (p.Ser1602Pro) | | | TATCGCATCCGAGCCACACA′] |
| 1699 | NM_002863.4(PYGL): c.2461T > | 5836 | PYGL | [′CTCCAGTGACCGAACAATTAAAGAAYATGC |
| | C (p.Tyr821His) | | | CCAAAACATCTGGAACGTGGA′] |
| 1700 | NM_003002.3(SDHD): c.284T > C | 6392 | SDHD | [′CCTTGCTCTGCGATGGACTATTCCCYGGCTG |
| | (p.Leu95Pro) | | | CAGCCCTCACTCTTCATGGT′] |
| 1701 | NM_004385.4(VCAN): | 1462 | VCAN | [′TAAGTATTGTGAAAACTCTGTTTTTHTCAGG |
| | c.4004 − 5T > C | | | TCGAATGAGTGATTTGAGTG′] |
| 1702 | NM_004519.3(KCNQ3): c.925T > | 3786 | KCNQ3 | [′TGAGACCTATGCAGATGCCCTGTGGYGGGG |
| | C (p.Trp309Arg) | | | CCTGGTGAGTCACTACCTTGG′] |
| 1703 | NM_004937.2(CTNS): c.473T > C | 1497 | CTNS | [′TCCACCCCCTGCAGTGTCATTGGTCYGAGCT |
| | (p.Leu158Pro) | | | TCGACTTCGTGGCTCTGAAC′] |
| 1704 | NM_006329.3(FBLN5): c.649T > | 10516 | FBLN5 | [′GAACGAGTGTGCCACCGAGAACCCCYGCGT |
| | C (p.Cys217Arg) | | | GCAAACCTGCGTCAACACCTA′] |
| 1705 | NM_006432.3(NPC2): c.295T > C | 10577 | NPC2 | [′TGATGGTTGTAAGAGTGGAATTAACYGCCCT |
| | (p.Cys99Arg) | | | ATCCAAAAAGACAAGACCTA′] |
| 1706 | NM_007375.3(TARDBP): | 23435 | TARDBP | [′CATGGTAAGTATATTGTAAAATACAYATGTA |
| | c.*83T > C | | | CTAAGAATTTTCAAAATTGG′] |
| 1707 | NM_013246.2(CLCF1): c.46T > C | −1 | — | [′CTCGTGGGGGATGTTAGCGTGCCTGYGCAC |
| | (p.Cys16Arg) | | | GGTGCTCTGGCACCTCCCTGC′] |
| 1708 | NM_013246.2(CLCF1): c.676T > | −1 | — | [′CCTGCACCTGGGGGCTCATGGCTTCYGACTT |
| | C (p.Ter226Arg) | | | CTGACCTTCTCCTCTTCGCT′] |
| 1709 | NM_024577.3(SH3TC2): | 79628 | SH3TC2 | [′CTGCCCTTTGCCGAGCGCCTGCAGCYCCTCT |
| | c.1982T > C (p.Leu661Pro) | | | CTGGACACCCTCCTGCCTCT′] |
| 1710 | NM_172107.2(KCNQ2): c.2T > C | 3785 | KCNQ2 | [′CCGGGGCGCCTCCCGCCAGGCACCAYGGTG |
| | (p.Met1Thr) | | | CAGAAGTCGCGCAACGGCGGC′] |
| 1711 | NM_004304.4(ALK): c.3749T > C | 238 | ALK | [′TGCATTTCCTTTCTTCCCAGAGACAYTGCTG |
| | (p.Ile1250Thr) | | | CCAGAAACTGCCTCTTGACC′] |
| 1712 | NM_000495.4(COL4A5): | 1287 | COL4A5 | [′TTCCTTCTCCTTTTCCTTTACCAGAYGTGCAG |
| | c.4690T > C (p.Cys1564Arg) | | | TATGTGAAGCTCCAGCTGT′] |
| 1713 | NM_000495.4(COL4A5): | 1287 | COL4A5 | [′CTTTTCCTTTACCAGATGTGCAGTAYGTGAA |
| | c.4699T > C (p.Cys1567Arg) | | | GCTCCAGCTGTGGTGATCGC′] |
| 1714 | NM_000495.4(COL4A5): | 1287 | COL4A5 | [′CAGTCAGACGATCCAGATTCCCCATYGTCCT |
| | c.4756T > C (p.Cys1586Arg) | | | CAGGGATGGGATTCTCTGTG′] |
| 1715 | NM_000495.4(COL4A5): | 1287 | COL4A5 | [′TAAGAAGCTTAAACTTCAAACAGCTYCTATC |
| | c.4803 + 121T > C | | | CAAGCACTGTGTTCCCCCTC′] |
| 1716 | NM_000495.4(COL4A5): | 1287 | COL4A5 | [′AGACTTGAGGACACGAATTAGCCGAYGTCA |
| | c.5032T > C (p.Cys1678Arg) | | | AGTGTGCATGAAGAGGACATA′] |
| 1717 | NM_005359.5(SMAD4): c.970T > | 4089 | SMAD4 | [′ATTTCCTATAGCTCCTGAGTATTGGYGTTCC |
| | C (p.Cys324Arg) | | | ATTGCTTACTTTGAAATGGA′] |
| 1718 | NM_005359.5(SMAD4): | 4089 | SMAD4 | [′GGACCCTTCTGGAGGAGATCGCTTTYGTTTG |
| | c.1087T > C (p.Cys363Arg) | | | GGTCAACTCTCCAATGTCCA′] |
| 1719 | NM_005359.5(SMAD4): | 4089 | SMAD4 | [′ATTGAAATTCACTTACACCGGGCCCBCCAGC |
| | c.1598T > C (p.Leu533Pro) | | | TCCTAGACGAAGTACTTCAT′] |
| 1720 | NM_020630.4(RET): c.1831T > C | 5979 | RET | [′TAAAGCTGGCTATGGCACCTGCAACNGCTTC |
| | (p.Cys611Arg) | | | CCTGAGGAGGAGAAGTGCTT′] |
| 1721 | NM_020630.4(RET): c.1888T > C | 5979 | RET | [′ACCACCCCCACCCACAGATCCACTGYGCGA |
| | (p.Cys630Arg) | | | CGAGCTGTGCCGCACGGTGAT′] |
| 1722 | NM_000060.3(BTD): c.248T > C | 686 | BTD | [′GCTCTCATCAGCCGCCAAGAGGCCTYGGAG |
| | (p.Leu83Ser) | | | CTCATGAACCAGAACCTTGAC′] |
| 1723 | NM_000060.3(BTD): c.445T > C | 686 | BTD | [′GAACCCATGCCTGGAGCCTCACCGCYTCAAT |
| | (p.Phe149Leu) | | | GACACAGAGGTGATTCCTGC′] |
| 1724 | NM_000060.3(BTD): c.743T > C | 686 | BTD | [′TTTGGCATCTTCACATGCTTTGATAYATTGTT |
| | (p.Ile248Thr) | | | CTTTGACCCTGCCATCAGA′] |
| 1725 | NM_000060.3(BTD): c.764T > C | 686 | BTD | [′GATATATTGTTCTTTGACCCTGCCAYCAGAG |
| | (p.Ile255Thr) | | | TCCTCAGAGACTACAAGGTG′] |
| 1726 | NM_000060.3(BTD): c.833T > C | 686 | BTD | [′ACTGCCTGGATGAACCAGCTCCCACYCTTGG |
| | (p.Leu278Pro) | | | CAGCAATTGAGATTCAGAAA′] |
| 1727 | NM_000060.3(BTD): c.1096T > C | 686 | BTD | [′CCATAGTAAGTTTTTAAAAATTTTGYCAGGC |
| | (p.Ser366Pro) | | | GATCCGTACTGTGAGAAGGA′] |
| 1728 | NM_000060.3(BTD): c.1214T > C | 686 | BTD | [′GAGATGATGTATGACAATTTCACCCYGGTCC |
| | (p.Leu405Pro) | | | CTGTCTGGGGAAAGGAAGGC′] |
| 1729 | NM_000060.3(BTD): c.1252T > C | 686 | BTD | [′GGGAAAGGAAGGCTATCTCCACGTCYGTTC |
| | (p.Cys418Arg) | | | CAATGGCCTCTGCTGTTATTT′] |
| 1730 | NM_000060.3(BTD): c.1267T > C | 686 | BTD | [′TCTCCACGTCTGTTCCAATGGCCTCYGCTGT |
| | (p.Cys423Arg) | | | TATTTACTTTACGAGAGGCC′] |
| 1731 | NM_000060.3(BTD): c.1459T > C | 686 | BTD | [′CACGGGGATATTTGAGTTTCACCTGYGGGGC |
| | (p.Trp487Arg) | | | AACTTCAGTACTTCCTATAT′] |
| 1732 | NM_000155.3(GALT): c.265T > C | 2592 | GALT | [′TCCTTGTCGGTAGGTGAATCCCCAGBACGAT |
| | (p.Tyr89His) | | | AGCACCTTCCTGTTTGACAA′] |
| 1733 | NM_000155.3(GALT): | 2592 | GALT | [′TGCAGCCTGATGCCCCCAGTCCAGGYAACCT |
| | c.328 + 2T > C | | | GGCTCCAACTGCTGCTGGGG′] |
| 1734 | NM_000155.3(GALT): c.336T > C | 2592 | GALT | [′GATACTCCTTTACCTCAGGACCCAGYGATCA |
| | (p.Ser112=) | | | TCCCCTTTTCCAAGCAAAGT′] |
| 1735 | NM_000155.3(GALT): c.350T > C | 2592 | GALT | [′TCAGGACCCAGTGATCATCCCCTTTYCCAAG |
| | (p.Phe117Ser) | | | CAAAGTCTGCTCGAGGAGTC′] |
| 1736 | NM_000155.3(GALT): c.374T > C | 2592 | GALT | [′TTCCAAGCAAAGTCTGCTCGAGGAGYCTGG |
| | (p.Val125Ala) | | | TAACTATGGATTTCCCCTCTT′] |
| 1737 | NM_000155.3(GALT): c.425T > C | 2592 | GALT | [′TGGTCGGATGTAACGCTGCCACTCAHGTCGG |
| | (p.Met142Thr) | | | TCCCTGAGATCCGGGCTGTT′] |
| 1738 | NM_000155.3(GALT): c.452T > C | 2592 | GALT | [′TCGGTCCCTGAGATCCGGGCTGTTGYTGATG |
| | (p.Val151Ala) | | | CATGGGCCTCAGTCACAGAG′] |
| 1739 | NM_000155.3(GALT): c.460T > C | 2592 | GALT | [′TGAGATCCGGGCTGTTGTTGATGCABGGGCC |
| | (p.Trp154Arg) | | | TCAGTCACAGAGGAGCTGGG′] |
| 1740 | NM_000155.3(GALT): c.482T > C | 2592 | GALT | [′GCATGGGCCTCAGTCACAGAGGAGCYGGGT |
| | (p.Leu161Pro) | | | GCCCAGTACCCTTGGGTGCAG′] |
| 1741 | NM_000155.3(GALT): c.499T > C | 2592 | GALT | [′AGAGGAGCTGGGTGCCCAGTACCCTYGGGT |
| | (p.Trp167Arg) | | | GCAGGTTTGTGAGGTCGCCCC′] |
| 1742 | NM_000155.3(GALT): | 2592 | GALT | [′GGTGCCCAGTACCCTTGGGTGCAGGYTTGTG |
| | c.507 + 2T > C | | | AGGTCGCCCCTTCCCCTGGA′] |
| 1743 | NM_000155.3(GALT): c.509T > C | 2592 | GALT | [′GAGCTCCGTATCCCTATCTGATAGAHCTTTG |
| | (p.Ile170Thr) | | | AAAACAAAGGTGCCATGATG′] |
| 1744 | NM_000155.3(GALT): c.584T > C | 2592 | GALT | [′TGACAGGTATGGGCCAGCAGTTTCCYGCCA |
| | (p.Leu195Pro) | | | GATATTGCCCAGCGTGAGGAG′] |
| 1745 | NM_000155.3(GALT): c.650T > C | 2592 | GALT | [′TATAAGAGTCAGCATGGAGAGCCCCYGCTA |
| | (p.Leu217Pro) | | | ATGGAGTACAGCCGCCAGGAG′] |
| 1746 | NM_000155.3(GALT): c.677T > C | 2592 | GALT | [′CTAATGGAGTACAGCCGCCAGGAGCYACTC |
| | (p.Leu226Pro) | | | AGGAAGGTGGGAGAGAGCCAA′] |
| 1747 | NM_000155.3(GALT): | 2592 | GALT | [′AGCCGCCAGGAGCTACTCAGGAAGGYGGGA |
| | c.687 + 2T > C | | | GAGAGCCAAGCCCTGTGTCCC′] |
| 1748 | NM_000155.3(GALT): c.745T > C | 2592 | GALT | [′AGTACTGGTCCCCTTCTGGGCAACAYGGCCC |
| | (p.Trp249Arg) | | | TACCAGACACTGCTGCTGCC′] |
| 1749 | NM_000155.3(GALT): c.967T > C | 2592 | GALT | [′CCATTGGCAGCTGCACGCTCATTACBACCCT |
| | (p.Tyr323His) | | | CCGCTCCTGCGCTCTGCCAC′] |
| 1750 | NM_000155.3(GALT): c.980T > C | 2592 | GALT | [′CACGCTCATTACTACCCTCCGCTCCYGCGCT |
| | (p.Leu327Pro) | | | CTGCCACTGTCCGGAAATTC′] |
| 1751 | NM_000155.3(GALT): c.1138T > | 2592 | GALT | [′GGACAGGGAGACAGCAACCATCGCCYGACC |
| | C (p.Ter380Arg) | | | ACGCCGACCACAGGGCCTTGA′] |
| 1752 | NM_207346.2(TSEN54): | 283989 | TSEN54 | [′AGAAGAGGGCTTCGTGGAGTTGAAGYCTCC |
| | c.277T > C (p.Ser93Pro) | | | CGCGGTGAGCGGCGGGCTCGG′] |
| 1753 | NM_016269.4(LEF1): c.181T > C | −1 | — | [′CATCAAGTCTTCCTTGGTGAACGAGYCTGAA |
| | (p.Ser61Pro) | | | ATCATCCCGGCCAGCAACGG′] |
| 1754 | NM_001928.2(CFD): c.640T > C | 1675 | CFD | [′GGGTGACTCCGGGGGCCCGCTGGTGYGCGG |
| | (p.Cys214Arg) | | | GGGCGTGCTCGAGGGCGTGGT′] |
| 1755 | NM_001458.4(FLNC): c.752T > C | 2318 | FLNC | [′CCCAACGTGGATGAGCATTCTGTTAYGACCT |
| | (p.Met251Thr) | | | ACCTGTCCCAGTTCCCCAAG′] |
| 1756 | NM_000781.2(CYP11A1): | 1583 | CYP11A1 | [′ATTTTTGGGGAGCGCCAGGGGATGCYGGAG |
| | c.665T > C (p.Leu222Pro) | | | GAAGTAGTGAACCCCGAGGCC′] |
| 1757- | NM_000782.4(CYP24A1): | 1591 | CYP24A1 | [′ATAATAATGATGGTTGccaacatgcgctgag |
| 1761 | c.1226T > C (p.Leu409Ser) | | | tgcttaaaatgtgccaggta′, |
| | | | | ′ataaataggtaaagggctttagaattgtgcac |
| | | | | catactcacaaccaccctg′, |
| | | | | ′cgcagcagattctgttattaaaccccttttac |
| | | | | agacgaagttgaggctcac′, |
| | | | | ′agagattatgtaactcactcaagacaacacag |
| | | | | ataaaagatgaaactgaat, |
| | | | | ′ACAAATTCTACTTACTCCTTTGGGTRAAGCAT |
| | | | | ATTCACCCAGAACTGTTGC′] |
| 1762 | NM_001145661.1(GATA2): | 2624 | GATA2 | [′CAACGGGGACCCTGTCTGCAACGCCYGTGG |
| | c.1117T > C (p.Cys373Arg) | | | CCTCTACTACAAGCTGCACAA′] |
| 1763 | NM_004523.3(KIF11): | 3832 | KIF11 | [′CAGGAACTTCACAACTTATTGGAGGYAATA |
| | c.2547 + 2T > C | | | ACTTTGTAAGTGGAACTTACT′] |
| 1764 | NM_000238.3(KCNH2): | 3757 | KCNH2 | [′GGGCGGCCCCTCCATCAAGGACAAGBATGT |
| | c.1831T > C (p.Tyr611His) | | | GACGGCGCTCTACTTCACCTT′] |
| 1765 | NM_002465.3(MYBPC1): | 4604 | MYBPC1 | [′GGAGGAAGAACCCCAGGTGGACGTAYGGGA |
| | c.706T > C (p.Trp236Arg) | | | GTTGCTGAAGAACGCGAAACC′] |
| 1766 | NM_002465.3(MYBPC1): | 4604 | MYBPC1 | [′CATTCCAAGACACCTGAAGCAAACCYATAT |
| | c.2566T > C (p.Tyr856His) | | | CCGCAGAGTTGGAGAAGCTGT′] |
| 1767 | NM_005211.3(CSF1R): c.2624T > | 1436 | CSF1R | [′AAACTGGTGAAGGATGGATACCAAAYGGCC |
| | C (p.Met875Thr) | | | CAGCCTGCATTTGCCCCAAAG′] |
| 1768 | NM_005211.3(CSF1R): c.2381T > | 1436 | CSF1R | [′TTGACCAATGGTCATGTGGCCAAGAYTGGG |
| | C (p.Ile794Thr) | | | GACTTCGGGCTGGCTAGGGAC′] |
| 1769 | NM_003392.4(WNT5A): | 7474 | WNT5A | [′GCCGCGGGACTGGCTCTGGGGCGGCYGCGG |
| | c.544T > C (p.Cys182Arg) | | | CGACAACATCGACTATGGCTA′] |
| 1770 | NM_005188.3(CBL): c.1150T > C | 867 | CBL | [′CTCCACATTCCAACTATGTAAAATABGTGCT |
| | (p.Cys384Arg) | | | GAAAATGATAAGGATGTAAA′] |
| 1771 | NM_005188.3(CBL): c.1186T > C | 867 | CBL | [′TGATAAGGATGTAAAGATTGAGCCCYGTGG |
| | (p.Cys396Arg) | | | ACACCTCATGTGCACATCCTG′] |
| 1772 | NM_006902.4(PRRX1): c.338T > | 5396 | PRRX1 | [′CAGCTGCAGGCTTTGGAGCGTGTCTYTGAGC |
| | C (p.Phe113Ser) | | | GGACACACTATCCTGATGCT′] |
| 1773 | NM_001111035.1(ACP5): | −1 | — | [′GCGGCGGCCAGGGAGGACTACGTGCYGGTG |
| | c.602T > C (p.Leu201Pro) | | | GCTGGCCACTACCCCGTGTGG′] |
| 1774 | NM_000329.2(RPE65): c.1102T > | 6121 | RPE65 | [′GGCTCCCCAACCTGAAGTTAGGAGAYATGT |
| | C (p.Tyr368His) | | | ACTTCCTTTGAATATTGACAA′] |
| 1775 | NM_003172.3(SURF1): c.679T > | 6834 | SURF1 | [′TGAGAACAATCCAGAAAGGAACCACYGGCA |
| | C (p.Trp227Arg) | | | TTATCGAGACCTGGAAGCTAT′] |
| 1776 | NM_002734.4(PRKARIA): | 5573 | PRKARIA | [′CATCCTCAAACGAAACATCCAGCAGYACAA |
| | c.1117T > C (p.Tyr373His) | | | CAGTTTTGTGTCACTGTCTGT′] |
| 1777 | NM_002734.4(PRKARIA): | 5573 | PRKARIA | [′CCATCTTTGCTTTCTCCAGGTGAAAYTGCAC |
| | c.980T > C (p.Ile327Thr) | | | TACTGATGAATCGTCCTCGT′] |
| 1778 | NM_003491.3(NAA10): c.109T > | 8260 | NAA10 | [′GAAATACTACTTCTACCATGGCCTTYCCTGG |
| | C (p.Ser37Pro) | | | CCCCAGGTGGGCAGCTTCTG′] |
| 1779 | NM_006306.3(SMC1A): | 8243 | SMC1A | [′GTGTTTGAAGAGTTTTGTCGGGAGAYTGGTG |
| | c.2351T> C (p.Ile784Thr) | | | TGCGCAACATCCGGGAGTTT′] |
| 1780 | NM_000377.2(WAS): c.814T > C | 7454 | WAS | [′CGACCCAGATCTGCGGAGTCTGTTCYCCAGG |
| | (p.Ser272Pro) | | | GCAGGAATCAGCGAGGCCCA′] |
| 1781 | NM_000377.2(WAS): c.881T > C | 7454 | WAS | [′ACCTCTAAACTTATCTACGACTTCAYTGAGG |
| | (p.Ile294Thr) | | | ACCAGGGTGGGCTGGAGGCT′] |
| 1782 | m.12338T > C | 4540 | MT-ND5 | [′TTGGTGCAACTCCAAATAAAAGTAAYAACC |
| | | | | ATGCACACTACTATAACCACC′] |
| 1783 | m.5814T > C | 4511 | MT-TC | [′TCGAATTTGCAATTCAATATGAAAAYCACCT |
| | | | | CGGAGCTGGTAAAAAGAGGC′] |
| 1784 | m.12201T > C | 4564 | MT-TH | [′GACAACAGAGGCTTACGACCCCTTAYTTACC |
| | | | | GAGAAAGCTCACAAGAACTG′] |
| 1785 | NM_001165899.1(PDE4D): | 5144 | PDE4D | [′GGAGATGACTTGATTGTGACTCCATBTGCTC |
| | c.494T > C (p.Phe165Ser) | | | AGGTAAGCACAGCTTGGTGA′] |
| 1786 | NM_139125.3(MASP1): | 5648 | MASP1 | [′GTTACCCGTGGTGCCTCACGCTGAGYGCAA |
| | c.1888T > C (p.Cys630Arg) | | | AACTAGCTATGAGTCCCGCTC′] |
| 1787 | NM_007315.3(STAT1): c.520T > | 6772 | STAT1 | [′TTTACAAGATGAATATGACTTCAAAYGCAA |
| | C (p.Cys174Arg) | | | AACCTTGCAGAACAGAGGTAA′] |
| 1788 | NM_053025.3(MYLK): c.5275T > | −1 | — | [′TGCTGTGAGAGCCATTGGAAGACTGYCCTCT |
| | C (p.Ser1759Pro) | | | ATGGCAATGATCTCAGGGCT′] |
| 1789 | NM_001235.3(SERPINH1): | 871 | SERPINH1 | [′TCACCCGTGGTGGTGGCCTCGTCGCYAGGGC |
| | c.233T > C (p.Leu78Pro) | | | TCGTGTCGCTGGGCGGCAAG′] |
| 1790 | NM_005921.1(MAP3K1): | 4214 | MAP3K1 | [′GAGGAACGAATGATCAGGGAGAAACBGAA |
| | c.566T > C (p.Leu189Pro) | | | GGCAACCTGTATGCCAGCCTGG′] |
| 1791 | NM_005359.5(SMAD4): | 4089 | SMAD4 | [′GTTGATGACCTTCGTCGCTTATGCAYACTCA |
| | c.1499T > C (p.Ile500Thr) | | | GGATGAGTTTTGTGAAAGGC′] |
| 1792 | NM_000287.3(PEX6): c.1601T > | 5190 | PEX6 | [′CTGTTGCTCACAGCTGTGGACCTTCYGGGCC |
| | C (p.Leu534Pro) | | | GGGACCGTGATGGGCTGGGT′] |
| 1793 | NM_004153.3(ORC1): c.266T > C | 4998 | ORC1 | [′AAGAAACGTGCTCGAGTACAGTGGTYTGTC |
| | (p.Phe89Ser) | | | CGATTCTGTGAAGTCCCTGCC′] |
| 1794 | NM_006225.3(PLCD1): c.562T > | 5333 | PLCD1 | [′CTCGGCTGTCCTGGCTCTGCAGGAGYGTGAC |
| | C (p.Cys188Arg) | | | CACTCCCAGACAGACTCCCT′] |
| 1795 | NM_021252.4(RAB18): c.619T > | 22931 | RAB18 | [′CTGTGGTGGTTATTGCTCTGTGTTAYAAACT |
| | C (p.Ter207Gln) | | | CTGGGAAATTCCATCTCTTG′] |
| 1796 | NM_002242.4(KCNJ13): | −1 | — | [′TGGTGTAATGGAGTGATAGTACGTTDGTGG |
| | c.722T > C (p.Leu241Pro) | | | AAAGATGAAGAATGGACATTC′] |
| 1797 | NM_003072.3(SMARCA4): | 6597 | SMARCA4 | [′CTGCAGCGAGTGCTCTACCGCCACAYGCAG |
| | c.3032T > C (p.Met1011Thr) | | | GCCAAGGGCGTGCTGCTGACT′] |
| 1798 | NM_013339.3(ALG6): c.391T > C | 29929 | ALG6 | [′GAGATTTCTTTTAAGCAACAACAGTRCAAAA |
| | (p.Tyr131His) | | | CCACTGCAGGTATGTAAATC′] |
| 1799 | NM_019885.3(CYP26B1): | 56603 | CYP26B1 | [′TTGCCTCTCTGCCTTCCAGGTCTTCYCCAAG |
| | c.436T > C (p.Ser146Pro) | | | ATCTTCAGCCACGAGGCCCT′] |
| 1800 | NM_020433.4(JPH2): c.421T > C | 57158 | JPH2 | [′GTTCACCAACGGCATGCGCCATGGCYACGG |
| | (p.Tyr141His) | | | AGTACGCCAGAGCGTGCCCTA′] |
| 1801 | NM_000199.3(SGSH): c.892T > C | 6448 | SGSH | [′CCCCAGCGTTTTGGGTGCTCCGGGGRTGACA |
| | (p.Ser298Pro) | | | CCAGTAAGGGTTCAGCAGTG′] |
| 1802 | NM_173170.1(IL36RN): c.80T > | 26525 | IL36RN | [′CTTTATCTGCATAATAACCAGCTTCYAGCTG |
| | C (p.Leu27Pro) | | | GAGGGCTGCATGCAGGGAAG′] |
| 1803 | NM_016599.4(MYOZ2): c.142T > | 51778 | MYOZ2 | [′CAGAGACATCATGTTGGAAGAATTAYCCCA |
| | C (p.Ser48Pro) | | | TCTCAGTAACCGTGGTGCCAG′] |
| 1804 | NM_020191.2(MRPS22): | 56945 | MRPS22 | [′CCAATAATTTTCAAGGAAGAAAATCYTAGG |
| | c.644T > C (p.Leu215Pro) | | | GTAAGGTGACTTAGGTTTTAT′] |
| 1805 | NM_004278.3(PIGL): c.500T > C | 9487 | PIGL | [′TTGTCCTATCCCTCCTCCAGGGCCCYGCACT |
| | (p.Leu167Pro) | | | CAGAAGGGAAGTTACCTAAA′] |
| 1806 | NM_022445.3(TPK1): c.119T > C | 27010 | TPK1 | [′ATTTTGTTCTTTCTATTTGAAGCTCYTTTAAG |
| | (p.Leu40Pro) | | | AGCCTGTGCCGATGGAGGT′] |
| 1807 | NM_020634.1(GDF3): c.914T > C | 9573 | GDF3 | [′TGTCCCTTCTCACTGACCATCTCTCYCAACA |
| | (p.Leu305Pro) | | | GCTCCAATTATGCTTTCATG′] |
| 1808 | NM_017882.2(CLN6): c.200T > C | 54982 | CLN6 | [′CCCATTCTTCCATTTGCTCCGCAGCYGGTAT |
| | (p.Leu67Pro) | | | TCCCTCTCGAGTGGTTTCCA′] |
| 1809 | NM_024513.3(FYCO1): | 79443 | FYCO1 | [′TTTGTGAGGTCCAGCACCTACAGCCYGATCC |
| | c.4127T > C (p.Leu1376Pro) | | | CCATCACTGTGGCCGAGGCA′] |
| 1810 | NM_006147.3(IRF6): c.65T > C | 3664 | IRF6 | [′CTGGTGGCCCAGGTGGATAGTGGCCYCTAC |
| | (p.Leu22Pro) | | | CCTGGGCTCATCTGGCTACAC′] |
| 1811 | NM_025132.3(WDR19): | 57728 | WDR19 | [′GGAAATGTTGGCATAGTGATGTCCTYGGAA |
| | c.2129T > C (p.Leu710Ser) | | | CAAATAAAGGTAAACAGCATG′] |
| 1812 | NM_025132.3(WDR19): c.20T > | 57728 | WDR19 | [′TTTATTTTTTAGCGTATTTTCTCACYGCTAGA |
| | C (p.Leu7Pro) | | | AAAGACTTGGCTTGGCGCA′] |
| 1813 | NM_153026.2(PRICKLE1): | 144165 | PRICKLE1 | [′AAGTAAAAAATACCAGTCTGATATGYACTG |
| | c.1414T > C (p.Tyr472His) | | | GGCACAGTCACAAGATGGACT′] |
| 1814 | NM_014874.3(MFN2): | 9927 | MFN2 | [′GTAGTCCTCAAGGTTTATAAGAATGWGAGT |
| | c.1392 + 2T > C | | | CATGGAGCAACAGGTCCTCTT′] |
| 1815 | NM_014874.3(MFN2): c.647T > C | 9927 | MFN2 | [′GAGCTGGACAGCTGGATTGACAAGTYTTGT |
| | (p.Phe216Ser) | | | CTGGATGCTGATGTGTTTGTG′] |
| 1816 | NM_016097.4(IER3IP1): | 51124 | IER3IP1 | [′GTAAACTCAATTGCAATTGTGTTACYTTTAT |
| | c.233T > C (p.Leu78Pro) | | | TATTTGGATGAATATCAGTG′] |
| 1817 | NM_018718.2(CEP41): c.107T > | 95681 | CEP41 | [′TTCGAGCTTCTCAGTATATTTAGTCRTACTGT |
| | C (p.Met36Thr) | | | TACCTAAAAAGAAAATAAG′] |
| 1818 | NM_022489.3(INF2): c.310T > C | 64423 | INF2 | [′CCTGCTGCAGCTCACCTGCGTCAGCYGCGTG |
| | (p.Cys104Arg) | | | CGCGCCGTCATGAACTCGCG′] |
| 1819 | NM_022489.3(INF2): c.383T > C | 64423 | INF2 | [′AGCAACCAGGGCTACGTGCGCCAGCYCTCC |
| | (p.Leu128Pro) | | | CAGGGTGAGCCGCAGTGTGGG′] |
| 1820 | NM_018713.2(SLC30A10): | 55532 | SLC30A10 | [′GGCGCGCTGAGCAACGCGGTCTTCCYCACC |
| | c.266T > C (p.Leu89Pro) | | | GCGCTCTGCTTCACCATCTTC′] |
| 1821 | NM_058246.3(DNAJB6): | 10049 | DNAJB6 | [′CAGTCCATTTGAATTTGGCTTCACAYTCCGT |
| | c.277T > C (p.Phe93Leu) | | | AACCCAGATGATGTCTTCAG′] |
| 1822 | NM_024753.4(TTC21B): | 79809 | TTC21B | [′CAAAAGAATTATCTTTGCTATGACCYGGCTG |
| | c.2384T > C (p.Leu795Pro) | | | AGCTCTTATTAAAATTGAAA′] |
| 1823 | NM_032446.2(MEGF10): | 84466 | MEGF10 | [′TGACTGCGACCACATTTCTGGGCAGYGTACT |
| | c.2320T > C (p.Cys774Arg) | | | TGCCGCACTGGATTCATGGG′] |
| 1824 | NM_032446.2(MEGF10): | 84466 | MEGF10 | [′CGTTCTCTGTGCTGAGACCTGCCAGYGTGTC |
| | c.976T > C (p.Cys326Arg) | | | AACGGAGGGAAGTGTTACCA′] |
| 1825 | NM_024027.4(COLEC11): | 78989 | COLEC11 | [′GAAGCGCTACGCGGACGCCCAGCTGYCCTG |
| | c.505T > C (p.Ser169Pro) | | | CCAGGGCCGCGGGGGCACGCT′] |
| 1826 | NM_000035.3(ALDOB): | 229 | ALDOB | [′GCCCCAGAAGAACCCGTGTGAACATRCTGT |
| | c.1027T > C (p.Tyr343His) | | | CCTTTGGCCGCCTGGCAGTTA′] |
| 1827 | NM_012338.3(TSPAN12): | 23554 | TSPAN12 | [′CCTTCTATCATAATACAGAGCCCAGRGCAGA |
| | c.734T > C (p.Leu245Pro) | | | GTAATGGTGAGAATCATGGC′] |
| 1828 | NM_001159772.1(CANT1): | 124583 | CANT1 | [′CTCCTTGCCCAGGCCGCCCACGTACRGACGC |
| | c.671T > C (p.Leu224Pro) | | | TCGTCCTTCACTGCCAGCCA′] |
| 1829 | NM_001006657.1(WDR35): | 57539 | WDR35 | [′TGGCATGTACGTAGTAGGCATCCAGYGGAA |
| | c.781T > C (p.Trp261Arg) | | | CCACATGGGCAGCGTGTTAGC′] |
| 1830 | NM_000420.2(KEL): c.1790T > C | 3792 | KEL | [′TCAACAGTACTGCCTGGGGGCTGCCYCGCCT |
| | (p.Leu597Pro) | | | GTGACAACCATGCCCTCCAG′] |
| 1831 | NM_001243473.1(B9D1): | 27077 | B9D1 | [′AGGGCCCAGGTCAGAATGAGGACCTRCCGG |
| | c.400 + 2T > C | | | CCAGGTGAGAAGGGCACGTGC′] |
| 1832 | NM_015175.2(NBEAL2): | 23218 | NBEAL2 | [′ATTGCAGTCCATGTAGTCAGAGTGCYGACCT |
| | c.1163T > C (p.Leu388Pro) | | | GCATCATGAGTGACTCCCCC′] |
| 1833 | NM_000196.3(HSD11B2): | 3291 | HSD11B2 | [′GCTGGCAGCTCGGCCCCGCCGCCGCYATTAC |
| | c.1012T > C (p.Tyr338His) | | | CCCGGCCAGGGCCTGGGGCT′] |
| 1834 | NM_024599.5(RHBDF2): | 79651 | RHBDF2 | [′GCTTACCGCCCCCCTCCCTTCCAGAYTGTGG |
| | c.557T > C (p.Ile186Thr) | | | ATCCGCTGGCCCGGGGCCGG′] |
| 1835 | NM_001256714.1(DNAAF3): | 352909 | DNAAF3 | [′GGCTCTGTGGATGGACGGCACCTGCYGCGG |
| | c.386T > C (p.Leul29Pro) | | | ACCCTGTCCCGAGCGAAGTTC′] |
| 1836 | NM_020894.2(UVSSA): c.94T > C | 57654 | UVSSA | [′GAAAATGAAGGAACTGAAGAAAATTYGCAA |
| | (p.Cys32Arg) | | | GTATGTCTTAGGGTTCAGTAA′] |
| 1837 | NM_004453.3(ETFDH): | 2110 | ETFDH | [′TTGTTTCCTCAGTCTATACCAAAACYCACCT |
| | c.1130T > C (p.Leu377Pro) | | | TTCCTGGTGGTTTACTAATT′] |
| 1838 | NM_024306.4(FA2H): c.707T > C | 79152 | FA2H | [′CTCATCGAGTACCTCATCCACCGCTYCCTGT |
| | (p.Phe236Ser) | | | TCCACATGAAGCCCCCCAGC′] |
| 1839 | NM_001004127.2(ALG11): | −1 | — | [′AAAATAAACATTCCATTTGATGAATYAAAG |
| | c.1142T > C (p.Leu381Ser) | | | AATTATTTGTCTGAAGCAACA′] |
| 1840 | NM_021167.4(GATAD1): | 57798 | GATAD1 | [′TCGGCTCAGAAACACTAAATACAAAYCTGC |
| | c.304T > C (p.Ser102Pro) | | | TCCGGCTGCTGAAAAGAAAGT′] |
| 1841 | NM_016042.3(EXOSC3): | 51010 | EXOSC3 | [′AGTATTTGGAATGAATGGAAGAATAYGGGT |
| | c.712T > C (p.Trp238Arg) | | | TAAGGCAAAAACCATCCAGCA′] |
| 1842 | NM_001018005.1(TPM1): | 7168 | TPM1 | [′CAGGTGGCCCGTAAGCTGGTCATCAYTGAG |
| | c.515T > C (p.Ile172Thr) | | | AGCGACCTGGAACGTGCAGAG′] |
| 1843 | NM_001018005.1(TPM1): | 7168 | TPM1 | [′GAGCTGGACCACGCTCTCAACGATAYGACT |
| | c.842T > C (p.Met281Thr) | | | TCCATGTAAACGTTCATCCAC′] |
| 1844 | NM_033360.3(KRAS): c.211T > C | 3845 | KRAS | [′GGAGTACAGTGCAATGAGGGACCAGYACAT |
| | (p.Tyr71His) | | | GAGGACTGGGGAGGGCTTTCT′] |
| 1845 | NM_006218.2(PIK3CA): | 5290 | PIK3CA | [′TTTTTCTTTGTTTTTTAAGGAACACYGTCCAT |
| | c.1258T > C (p.Cys420Arg) | | | TGGCATGGGGAAATATAAA′] |
| 1846 | NM_006265.2(RAD21): | −1 | — | [′TGAATCTATCAGTTTGCTTGAGTTAYGTCGA |
| | c.1753T > C (p.Cys585Arg) | | | AATACGAACAGAAAACAAGC′] |
| 1847 | NM_000222.2(KIT): c.1859T > C | 3815 | KIT | [′ATTAAGTCAGATGCGGCCATGACTGYCGCT |
| | (p.Val620Ala) | | | GTAAAGATGCTCAAGCGTAAG′] |
| 1848 | NM_000076.2(CDKNIC): | 1028 | CDKNIC | [′CGCGCGCTGTCGCCCGCAGATTTCTYCGCCA |
| | c.827T > C (p.Phe276Ser) | | | AGCGCAAGAGATCAGCGCCT′] |
| 1849 | NM_005691.3(ABCC9): | 10060 | ABCC9 | [′AGACTATTGGCTGGCCACATGGACAYCGGA |
| | c.3058T > C (p.Ser1020Pro) | | | GTACAGTATAAACAATACTGG′] |
| 1850 | NM_012343.3(NNT): c.2930T > C | 23530 | NNT | [′ATGCCTGGTCAGCTTAATGTGCTGCYGGCTG |
| | (p.Leu977Pro) | | | AGGCTGGTGTGCCATATGAC′] |
| 1851 | NM_024110.4(CARD14): | 79092 | CARD14 | [′GTGCTGCTGCGGCGGTGCCAGCAGCYGCAG |
| | c.467T > C (p.Leu156Pro) | | | GAGCACCTGGGCCTGGCCGAG′] |
| 1852 | NM_000492.3(CFTR): c.1400T > | 1080 | CFTR | [′GATGGGTTTTATTTCCAGACTTCACYTCTAA |
| | C (p.Leu467Pro) | | | TGGTGATTATGGGAGAACTG′] |
| 1853 | NM_000518.4(HBB): c.2T > C | 3043 | HBB | [′CACTAGCAACCTCAAACAGACACCANGGTG |
| | (p.Met1Thr) | | | CATCTGACTCCTGAGGAGAAG′] |
| 1854 | NM_000518.4(HBB): c.*110T > C | 3043 | HBB | [′CCTTGAGCATCTGGATTCTGCCTAAHAAAAA |
| | | | | ACATTTATTTTCATTGCAAT′] |
| 1855 | NM_000518.4(HBB): | 3043 | HBB | [′AGTTGGTGGTGAGGCCCTGGGCAGGNTGGT |
| | c.92 + 2T > C | | | ATCAAGGTTACAAGACAGGTT′] |
| 1856 | NM_000206.2(IL2RG): c.455T > | 3561 | IL2RG | [′ACCACATATGCACACATATCTCCAGYGATCC |
| | C (p.Val152Ala) | | | CCTGGGCTCCAGAGAACCTA′] |
| 1857 | NM_000244.3(MEN1): c.518T > C | 4221 | MEN1 | [′GCTGTGGTTGGGGCCTGCCAGGCCCYGGGT |
| | (p.Leu173Pro) | | | CTCCGGGATGTCCACCTCGCC′] |
| 1858 | NM_000256.3(MYBPC3): | 4607 | MYBPC3 | [′CCGCCATCGTAGGCAGGCGGCTCCCVCTGTA |
| | c.2374T > C (p.Trp792Arg) | | | CTGTGCAGGAGTCCTCTCCC′] |
| 1859 | NM_002501.3(NFIX): c.179T > C | 4784 | NFIX | [′GAGGAGCGGGCGGTGAAGGACGAGCYGCTG |
| | (p.Leu60Pro) | | | GGCGAGAAGCCCGAGATCAAG′] |
| 1860 | NM_005022.3(PFN1): c.341T > C | 5216 | PFN1 | [′CCTCCTCCAGCGCTAGTCCTGCTGAYGGGCA |
| | (p.Met114Thr) | | | AAGAAGGTGTCCACGGTGGT′] |
| 1861 | NM_001172567.1(MYD88): | 4615 | MYD88 | [′CTTGCAGGTGCCCATCAGAAGCGACYGATC |
| | c.818T > C (p.Leu273Pro) | | | CCCATCAAGTACAAGGCAATG′] |
| 1862 | NM_001161581.1(POC1A): | 25886 | POCIA | [′GCCAGTGATGACAAGACTGTTAAGCYGTGG |
| | c.398T > C (p.Leu133Pro) | | | GACAAGAGCAGCCGGGAATGT′] |
| 1863 | NM_005270.4(GLI2): c.4663T > C | 2736 | GLI2 | [′CCCCCGAAACTCCTTGACCCTGCCCYCCATC |
| | (p.Ser1555Pro) | | | CCCGCAGGCATCAGCAACAT′] |
| 1864 | NM_152296.4(ATP1A3): | 478 | ATP1A3 | [′CTCCTCCGCCTAGGTCCCTGCCATCYCACTG |
| | c.2431T > C (p.Ser811Pro) | | | GCGTACGAGGCTGCCGAAAG′] |
| 1865 | NM_022787.3(NMNAT1): | 64802 | NMNAT1 | [′GAGAAACACTGCAGAAGCTAAGACAYAGGA |
| | c.838T > C (p.Ter280Gln) | | | ATTCTACAGCATGATATTTCA′] |
| 1866 | NM_005340.6(HINT1): c.250T > | 3094 | HINT1 | [′ACACTTAATGATTGTTGGCAAGAAAYGTGCT |
| | C (p.Cys84Arg) | | | GCTGATCTGGGCCTGAATAA′] |
| 1867 | NM_000155.3(GALT): c.416T > C | 2592 | GALT | [′TTCCACCCCTGGTCGGATGTAACGCYGCCAC |
| | (p.Leu139Pro) | | | TCATGTCGGTCCCTGAGATC′] |
| 1868 | NM_000155.3(GALT): c.386T > C | 2592 | GALT | [′CTCCCGTCACCACCCAGTAAGGTCAYGTGCT |
| | (p.Met129Thr) | | | TCCACCCCTGGTCGGATGTA′] |
| 1869 | NM_000155.3(GALT): c.680T > C | 2592 | GALT | [′ATGGAGTACAGCCGCCAGGAGCTACYCAGG |
| | (p.Leu227Pro) | | | AAGGTGGGAGAGAGCCAAGCC′] |
| 1870 | NM_007294.3(BRCA1): c.4986 + | 672 | BRCA1 | [′GCCTGACCCCAGAAGAATTTGTGAGBGTATC |
| | 6T > C | | | CATATGTATCTCCCTAATGA′] |
| 1871 | NM_007294.3(BRCA1): c.5074 + | 672 | BRCA1 | [′CTACTCATGTTGTTATGAAAACAGGYATACC |
| | 2T > C | | | AAGAACCTTTACAGAATACC′] |
| 1872 | NM_007294.3(BRCA1): | 672 | BRCA1 | [′GGTTTCTTTCAGCATGATTTTGAAGBCAGAG |
| | c.5207T > C (p. Val1736Ala) | | | GAGATGTGGTCAATGGAAGA′] |
| 1873 | NM_002437.4(MPV17): | 4358 | MPV17 | [′TGAAGCCCTGTTGAGGGGAGAACTTRCCAC |
| | c.186 + 2T > C | | | AAAGCCACAGCCCAGGGACAC′] |
| 1874 | NM_002769.4(PRSS1): c.116T > | −1 | — | [′GAGGAGAATTCTGTCCCCTACCAGGYGTCCC |
| | C (p.Val39Ala) | | | TGAATTCTGGCTACCACTTC′] |
| 1875 | NM_004211.3(SLC6A5): | 9152 | SLC6A5 | [′GATTTTCTTCTCTTTATCTGCTGCAYGGGGA |
| | c.1444T > C (p.Trp482Arg) | | | GGCCTGATCACTCTCTCTTC′] |
| 1876 | NM_005211.3(CSF1R): c.2297T > | 1436 | CSF1R | [′TTCTCCAGCCAAGTAGCCCAGGGCAYGGCC |
| | C (p.Met766Thr) | | | TTCCTCGCTTCCAAGAATGTG′] |
| 1877 | NM_005211.3(CSF1R): c.2546T > | 1436 | CSF1R | [′TATGGCATCCTCCTCTGGGAGATCTYCTCAC |
| | C (p.Phe849Ser) | | | TTGGTGAGCCACTGGGCCCA′] |
| 1878 | NM_005211.3(CSF1R): c.2603T > | 1436 | CSF1R | [′CTGGTGAACAGCAAGTTCTATAAACBGGTG |
| | C (p.Leu868Pro) | | | AAGGATGGATACCAAATGGCC′] |
| 1879 | NM_006796.2(AFG3L2): | 10939 | AFG3L2 | [′ATTTTTCAGATTGTTCAGTTTGGCABGAATG |
| | Tc.1997 > C (p.Met666Thr) | | | AAAAGGTTGGGCAAATCTCC′] |
| 1880 | NM_018713.2(SLC30A10): | 55532 | SLC30A10 | [′GTAAGTGGAAAGATTATTGCCACCCYGCAC |
| | c.1046T > C (p.Leu349Pro) | | | ATCAAGTATCCTAAGGACAGG′] |
| 1881 | NM_018713.2(SLC30A10): | 55532 | SLC30A10 | [′GCGGAGGGCTGTGTCCCCGGCGCTTYCGGG |
| | c.500T > C (p.Phe 167Ser) | | | GGGCCTCAGGGCGCGGAGGAC′] |
| 1882 | NM_000060.3(BTD): c.212T > C | 686 | BTD | [′TATGAGCATCCATCCATCCTGAGTCYGAACC |
| | (p.Leu71Pro) | | | CTCTGGCTCTCATCAGCCGC′] |
| 1883 | NM_000495.4(COL4A5): | 1287 | COL4A5 | [′TTTCCTGGTTTACAGGGTCCTCCAGYAAGTT |
| | c.438 + 2T > C | | | ATAAAATTTGGGATTATGAT′] |
| 1884 | NM_000282.3(PCCA): c.491T > C | 5095 | PCCA | [′CAGGCAGCAGAAGATGTCGTTTTCAYTGGA |
| | (p.Ile164Thr) | | | CCTGACACACATGCTATTCAA′] |
| 1885 | NM_000532.4(PCCB): c.1556T > | 5096 | PCCB | [′ACACGTGCCCGAATCTGCTGTGACCYGGAT |
| | C (p.Leu519Pro) | | | GTCTTGGCCAGCAAGAAGGTA′] |
| 1886 | NM_001099274.1(TINF2): | 26277 | TINF2 | [′CATAAGGAGCGCCCCACAGTCATGCYGTTTC |
| | c.860T > C (p.Leu287Pro) | | | CCTTTAGGAATCTCGGCTCA′] |
| 1887 | NM_001099274.1(TINF2): | 26277 | TINF2 | [′TAAGGAGCGCCCCACAGTCATGCTGYTTCCC |
| | c.862T > C (p.Phe288Leu) | | | TTTAGGAATCTCGGCTCACC′] |
| 1888 | NM_001363.4(DKC1): c.1049T > | 1736 | DKC1 | [′TCAATGCCTGTAGCTATTGCATTAAYGACCA |
| | C (p.Met350Thr) | | | CAGCGGTCATCTCTACCTGC′] |
| 1889 | NM_001363.4(DKC1): c.1193T > | 1736 | DKC1 | [′AAGCTGATGATCAAGCAGGGCCTTCYGGAC |
| | C (p.Leu398Pro) | | | AAGCATGGGAAGCCCACAGAC′] |
| 1890 | NM_004614.4(TK2): | 7084 | TK2 | [′CAGGAAAAAGAGAAAAAATCAGTGGYAAG |
| | c.156 + 2T > C | | | TCCCTCTTTTATGTGTACTCTC′] |
| 1891 | NM_004614.4(TK2): c.644T > C | 7084 | TK2 | [′GAATACCTGGAAGCAATTCACCATCYCCAT |
| | (p.Leu215Pro) | | | GAGGAGTGGCTCATCAAAGGC′] |
| 1892 | NM_024312.4(GNPTAB): | 79158 | GNPTAB | [′ATCGAAGGGCTGTCCCAGAAGTTTAYTTACC |
| | c.1208T > C (p.Ile403Thr) | | | TAAATGATGATGTCATGTTT′] |
| 1893 | NM_024312.4(GNPTAB): | 79158 | GNPTAB | [′TTTGCCTTCTCTTATTTTTATTATCYCATGAG |
| | c.3002T > C (p.Leu1001Pro) | | | TGCAGTGCAGCCACTGAAT′] |
| 1894 | NM_198253.2(TERT): c.3043T > | 7015 | TERT | [′CCGCCATCCTCTCAGGTTTCACGCAYGTGTG |
| | C (p.Cys1015Arg) | | | CTGCAGCTCCCATTTCATCA′] |
| 1895 | NM_207352.3(CYP4V2): | 285440 | CYP4V2 | [′TACAACTGCAGCTGCAATAAACTGGYCCTTA |
| | c.1021T > C (p.Ser341Pro) | | | TACCTGTTGGGTTCTAACCC′] |
| 1896 | NM_207352.3(CYP4V2): | 285440 | CYP4V2 | [′TGCTCAAAGTAATGATGATTCCGAGYATGTC |
| | c.655T > C (p.Tyr219His) | | | CGTGCAGTTTATAGGTAAAT′] |
| 1897 | NM_000335.4(SCN5A): | 6331 | SCN5A | [′GCCAAGCCCAACCAGATAAGCCTCAYCAAC |
| | c.5504T > C (p.Ile1835Thr) | | | ATGGACCTGCCCATGGTGAGT′] |
| 1898 | NM_001111.4(ADAR): c.2615T > | 103 | ADAR | [′CTCGGCCGCAAGATTCTGGCCGCCAYCATTA |
| | C (p.Ile872Thr) | | | TGAAAAAAGACTCTGAGGAC′] |
| 1899 | NM_004281.3(BAG3): c.1385T > | 9531 | BAG3 | [′ATCGAAGAGTATTTGACCAAAGAGCYGCTG |
| | C (p.Leu462Pro) | | | GCCCTGGATTCAGTGGACCCC′] |
| 1900 | NM_183075.2(CYP2U1): | 113612 | CYP2U1 | [′TTTTATGTCACGAGGCCTAGAAATCYGTCTG |
| | c.784T> C (p.Cys262Arg) | | | AACAGTCAAGTCCTCCTGGT′] |
| 1901 | NM_006177.3(NRL): c.287T > C | 4901 | NRL | [′TTGGGGCTGAGTCCTGAAGAGGCCAYGGAG |
| | (p.Met96Thr) | | | CTGCTGCAGGGTCAGGGCCCA′] |
| 1902 | NM_000344.3(SMN1): c.388T > C | 6606 | SMN1 | [′AACCTGTGTTGTGGTTTACACTGGAYATGGA |
| | (p.Tyr130His) | | | AATAGAGAGGAGCAAAATCT′] |
| 1903 | NM_152692.4(C1GALT1C1): | 29071 | C1GALT1C1 | [′AGGAGGAATTGTCTTAAGTGTAGAAYCAAT |
| | c.577T > C (p.Ser193Pro) | | | GAAAAGACTTAACAGCCTTCT′] |
| 1904 | NM_024531.4(SLC52A2): | 79581 | SLC52A2 | [′TTAGCACTGGCCTTTGTGCTGGCACYGGCAT |
| | Tc.368 > C (p.Leu123Pro) | | | GCTGTGCCTCGAATGTCACT′] |
| 1905 | NM_001253816.1(SLC52A2): | 79581 | SLC52A2 | [′GCTCACTGCAGGTCCTTGGCAGGGCYGGGC |
| | c.1016T > C (p.Leu339Pro) | | | GGCCTCTCTCTGCTGGGCGTG′] |
| 1906 | NM_002609.3(PDGFRB): | 5159 | PDGFRB | [′TCGGAGCTGAAGATCATGAGTCACCYTGGG |
| | c.1973T > C (p.Leu658Pro) | | | CCCCACCTGAACGTGGTCAAC′] |
| 1907 | NM_020822.2(KCNT1): | 57582 | KCNT1 | [′CAACAGCTATGAAGACGCCAAGGCCYACGG |
| | c.2386T > C (p.Tyr796His) | | | GTTCAAGAACAAGCTGATCAT′] |
| 1908 | NM_001369.2(DNAH5): | 1767 | DNAH5 | [′ACTATAGATCATTTTAATTGCATTTRTAAGT |
| | c.1121T > C (p.Ile374Thr) | | | GTAGGAATAGCATCCATCAT′] |
| 1909 | NM_017802.3(DNAAF5): | 54919 | DNAAF5 | [′GTCCAGTACCTGTACCGAGAGTTGCYGGTTC |
| | c.2384T > C (p.Leu795Pro) | | | ACCTTGACGATCCAGAGAGG′] |
| 1910 | NM_006383.3(CIB2): c.272T > C | 10518 | CIB2 | [′GAGGGGAACCTCACTTTCAACGACTYTGTG |
| | (p.Phe91Ser) | | | GACATGTTTTCCGTGCTCTGC′] |
| 1911 | NM_006383.3(CIB2): c.368T > C | 10518 | CIB2 | [′ACAGACTTCAACACTGACAACTTCAYCTGCA |
| | (p.Ile123Thr) | | | AGGAGGACCTGGAGCTGACG′] |
| 1912 | NM_005334.2(HCFC1): | 3054 | HCFC1 | [′TTAGTTGTTACTTCTTCACACAAGAYGGCGG |
| | c.−970T > C | | | CTCCCAGGGAGGAGGCATGA′] |
| 1913 | NM_178012.4(TUBB2B): | 347733 | TUBB2B | [′GGAGCCGAGCTGGTCGACTCGGTCCYGGAT |
| | c.350T > C (p.Leu117Pro) | | | GTGGTGAGGAAGGAGTCAGAG′] |
| 1914 | NM_000431.3(MVK): c.764T > C | 4598 | MVK | [′GTGGCTGGCGTCAGAAACAGGCTGCYCAAG |
| | (p.Leu255Pro) | | | GTGACTCTTGTTCCCTTCTTG′] |
| 1915 | NM_000431.3(MVK): c.122T > C | 4598 | MVK | [′TTGAACTTGAGAACATTCCTCCGGCYTCAAC |
| | (p.Leu41Pro) | | | CCCACAGCAATGGGAAAGTG′] |
| 1916 | NM_000431.3(MVK): | 4598 | MVK | [′GTGGCATCACACTCCTCAAGCCAGGYATCCC |
| | c.1039 + 2T > C | | | GGGGGTAGGTGGGCCAGGCT′] |
| 1917 | NM_000431.3(MVK): c.1094T > | 4598 | MVK | [′AAGCAGGCCCTGACCAGCTGTGGCTYTGAC |
| | C (p.Phe365Ser) | | | TGCTTGGAAACCAGCATCGGT′] |
| 1918 | NM_004055.4(CAPN5): c.731T > | 726 | CAPN5 | [′ACAGCAGCTGACATGGAGGCCCGCCYGGCG |
| | C (p.Leu244Pro) | | | TGCGGCCTGGTAAAGGGCCAC′] |
| 1919 | NM_133497.3(KCNV2): c.491T > | 169522 | KCNV2 | [′GCCGTCTTCCAGCTGGTCTACAATTYCTACC |
| | C (p.Phe164Ser) | | | TGTCCGGGGTGCTGCTGGTG′] |
| 1920 | NM_006567.3(FARS2): c.986T > | 10667 | FARS2 | [′ATGATCCTCTACGACATCCCTGATAYCCGTC |
| | C (p.Ile329Thr) | | | TCTTCTGGTGTGAGGACGAG′] |
| 1921 | NM_020376.3(PNPLA2): | 57104 | PNPLA2 | [′GCCTGCGCTTTCTGCAGCGGAACGGYGCGC |
| | c.757 + 2T > C | | | GGACCCGGGCGGGAGAGGGCG′] |
| 1922 | NM_018344.5(SLC29A3): | 55315 | SLC29A3 | [′TATGAGGAACTCCCAGGCACTGATAYCAGG |
| | c.607T > C (p.Ser203Pro) | | | TGAGAGCCAGGGTCCGGGCAG′] |
| 1923 | NM_012275.2(IL36RN): | 26525 | IL36RN | [′TGCAGGGAAGGTCATTAAAGGTTGGYGATG |
| | c.115 + 6T > C | | | AAACATGACCCACTTTCCTTG′] |
| 1924 | m.9191T > C | 4508 | MT-ATP6 | [′TTCACACTTCTAGTAAGCCTCTACCYGCACG |
| | | | | ACAACACATAATGACCCACC′] |
| 1925 | NM_000108.4(DLD): c.140T > C | 1738 | DLD | [′GTAGTTGATGCTGATGTAACAGTTAYAGGTT |
| | (p.Ile47Thr) | | | CTGGTCCTGGAGGATATGTT′] |
| 1926 | NM_000080.3(CHRNE): c.223T > | −1 | — | [′AGAGGAGACTCTCACCACTAGCGTCYGGAT |
| | C (p.Trp75Arg) | | | TGGAATCGTGAGTCAAATCTG′] |
| 1927 | NM_004376.5(COX15): | 1355 | COX15 | [′TGCCATTACAGTGCTCTACTTCCTCBCTCGG |
| | c.1030T > C (p.Ser344Pro) | | | AGAATTCCCCTTCCTAGAAG′] |
| 1928 | NM_004333.4(BRAF): c.1403T > | 673 | BRAF | [′GGACAAAGAATTGGATCTGGATCATYTGGA |
| | C (p.Phe468Ser) | | | ACAGTCTACAAGGGAAAGTGG′] |
| 1929 | NM_004333.4(BRAF): c.1454T > | 673 | BRAF | [′TTAGGTGATGTGGCAGTGAAAATGTYGAAT |
| | C (p.Leu485Ser) | | | GTGACAGCACCTACACCTCAG′] |
| 1930 | NM_005188.3(CBL): c.1201T > C | 867 | CBL | [′GATTGAGCCCTGTGGACACCTCATGYGCAC |
| | (p.Cys401Arg) | | | ATCCTGTCTTACATCCTGGCA′] |
| 1931 | NM_002834.3(PTPN11): c.211T > | 5781 | PTPN11 | [′CTATGACCTGTATGGAGGGGAGAAABTTGC |
| | C (p.Phe71Leu) | | | CACTTTGGCTGAGTTGGTCCA′] |
| 1932 | NM_002834.3(PTPN11): c.853T > | 5781 | PTPN11 | [′AAATAGATATAAAAACATCCTGCCCBGTAA |
| | C (p.Phe285Leu) | | | GTATCAATATTCCGCTCAGTA′] |
| 1933 | NM_002880.3(RAF1): c.769T > C | 5894 | RAF1 | [′AGGTTCCCTCTCCCAGAGGCAGAGGYCGAC |
| | (p.Ser257Pro) | | | ATCCACACCTAATGTCCACAT′] |
| 1934 | NM_002880.3(RAF1): c.1423T > | 5894 | RAF1 | [′AGTCCTTAACAAGCATTGAGATATAYTTCTC |
| | C (p.Phe475Leu) | | | CATGAAGGCTTAACAGTGAA′] |
| 1935 | NM_005633.3(SOS1): c.806T > C | 6654 | SOS1 | [′GGCCATATAGAAGATACAGTAGAAABGACA |
| | (p.Met269Thr) | | | GATGAAGGCAGTCCCCATCCA′] |
| 1936 | NM_005633.3(SOS1): c.1649T > | 6654 | SOS1 | [′ATATCTTTACAGTACCGGAGTACACYGGAA |
| | C (p.Leu550Pro) | | | AGGATGCTTGATGTAACAATG′] |
| 1937 | NM_005633.3(SOS1): c.2104T > | 6654 | SOS1 | [′TCGGCACTGGGTAGAGCACCACTTCYATGAT |
| | C (p.Tyr702His) | | | TTTGAAAGAGATGCATATCT′] |
| 1938 | NM_002755.3(MAP2K1): | 5604 | MAP2K1 | [′CAACTCTCCGTACATCGTGGGCTTCYATGGT |
| | c.388T > C (p.Tyr130His) | | | GCGTTCTACAGCGATGGCGA′] |
| 1939 | NC_012920.1: m.7505T > C | 4574 | MT-TS1 | [′TTTCAAGCCAACCCCATGGCCTCCAYGACTT |
| | | | | TTTCAAAAAGGTATTAGAAA′] |
| 1940 | NM_000084.4(CLCN5): c.674T > | 1184 | CLCN5 | [′GTGGCTTGCTGCTGTGGGAACATCCYGTGCC |
| | C (p.Leu225Pro) | | | ACTGCTTCAACAAATACAGG′] |
| 1941 | NM_000530.6(MPZ): c.89T > C | 4359 | MPZ | [′GCAGTGCTGTCCCCGGCCCAGGCCAYCGTG |
| | (p.Ile30Thr) | | | GTTTACACCGACAGGGAGGTC′] |
| 1942 | NM_000530.6(MPZ): c.244T > C | 4359 | MPZ | [′CCCTCATTCCTCATAGATCTTCCACYATGCC |
| | (p.Tyr82His) | | | AAGGGACAACCCTACATTGA′] |
| 1943 | NM_000530.6(MPZ): c.266T > C | 4359 | MPZ | [′CACTATGCCAAGGGACAACCCTACAHTGAC |
| | (p.Ile89Thr) | | | GAGGTGGGGACCTTCAAAGAG′] |
| 1944 | NM_000748.2(CHRNB2): | 1141 | CHRNB2 | [′TACCTCATGTTCACCATGGTGCTTGYCACCT |
| | c.923T > C (p.Val308Ala) | | | TCTCCATCGTCACCAGCGTG′] |
| 1945 | NM_003611.2(OFD1): | 8481 | OFD1 | [′CGGGGTATACTGGATACACTCAAGGYATCG |
| | c.111 + 2T > C | | | GATTTAGGCGTATCTGTGTCA′] |
| 1946 | NM_003611.2(OFD1): c.274T > C | 8481 | OFD1 | [′AAGATGTGGCTATGAATATTCACTTYCTGTT |
| | (p.Ser92Pro) | | | TTCTTTCCAGAAAGTGGTTT′] |
| 1947 | NM_024408.3(NOTCH2): | 4853 | NOTCH2 | [′GCCCCACCCTGTGACAGGTCTCCTGYGTCAT |
| | c.1117T > C (p.Cys373Arg) | | | CTGGATGATGCATGCATCAG′] |
| 1948 | NM_024408.3(NOTCH2): | 4853 | NOTCH2 | [′TCTGGATAAGATTGGAGGCTTCACAYGTCTG |
| | c.1438T > C (p.Cys480Arg) | | | TGCATGCCAGGTAAATGGGC′] |
| 1949 | NM_000540.2(RYR1): c.9242T > | 6261 | RYR1 | [′CCCCGCTGCCCTTCTAGGACAGTGAYGAAGT |
| | C (p.Met3081Thr) | | | CAGGCCCTGAGATCGTGAAG′] |
| 1950 | NM_000096.3(CP): c.650T > C | 1356 | CP | [′AAAGAAAAACATATTGACCGAGAATYTGTG |
| | (p.Phe217Ser) | | | GTGATGTTTTCTGTGGTGGAT′] |
| 1951 | NM_000096.3(CP): c.548T > C | 1356 | CP | [′GTGACTAGGATTTACCATTCCCACABTGATG |
| | (p.Ile183Thr) | | | CTCCAAAAGATATTGCCTCA′] |
| 1952 | NM_000540.2(RYR1): c.1205T > | 6261 | RYR1 | [′CAGGAGGAGTCCCAGGCCGCCCGCAYGATC |
| | C (p.Met402Thr) | | | CACAGCACCAATGGCCTATAC′] |
| 1953 | NM_006517.4(SLC16A2): | 6567 | SLC16A2 | [′TTCGGGGGCCTTATCGTCGTCTGTCYTTTCCT |
| | c.1253T > C (p.Leu418Pro) | | | GGGCCTTTGCGATGGCTTC′] |
| 1954 | NM_000096.3(CP): c.1123T > C | 1356 | CP | [′CCGTGGGAAGCATGTTAGACACTACYACAT |
| | (p.Tyr375His) | | | TGCCGCTGAGGAAATCATCTG′] |
| 1955 | NM_002739.3(PRKCG): c.391T > | 5582 | PRKCG | [′CGGGCTTGTGCACCAGGGCATGAAAHGCTC |
| | C (p.Cys131Arg) | | | CTGTGAGTGACCTGGGCCTTG′] |
| 1956 | NM_000116.4(TAZ): c.310T > C | 6901 | TAZ | [′GACCCCTGCAGCTGCAGACATCTGCYTCACC |
| | (p.Phe104Leu) | | | AAGGAGCTACACTCCCACTT′] |
| 1957 | NM_000138.4(FBN1): c.2341T > | 2200 | FBN1 | [′CAGTCTCCTTTGTGACAATGGACAAYGTAGA |
| | C (p.Cys781Arg) | | | AATACTCCTGGAAGTTTTGT′] |
| 1958 | NM_000138.4(FBN1): c.4222T > | 2200 | FBN1 | [′CTTTCTGGCTGTAGACCTTGATGAGYGCTCT |
| | C (p.Cys1408Arg) | | | GAGAACCTGAATCTCTGTGG′] |
| 1959 | NM_000256.3(MYBPC3): | 4607 | MYBPC3 | [′GTAGCACGGAGCTCTTTGTGAAAGGYGGGC |
| | c.1351 + 2T > C | | | CTGGGACCTGAGGATGTGGGA′] |
| 1960 | NM_000256.3(MYBPC3): | 4607 | MYBPC3 | [′GACGCGGAAGTAGTAGCCATTGCCARTGAT |
| | c.3392T > C (p.Ile1131Thr) | | | GAGCTCTGGCACCACGCAGTG′] |
| 1961 | NM_000256.3(MYBPC3): | 4607 | MYBPC3 | [′CCTCCTATCAGCCTTCCGCCGCACGYGAGTG |
| | c.821 + 2T > C | | | GCCATCCTCAGGGCCTGGGG′] |
| 1962 | NM_000257.3(MYH7): c.1370T > | 4625 | MYH7 | [′ACCAAGCAGCCACGCCAGTACTTCAYAGGA |
| | C (p.Ile457Thr) | | | GTCCTGGACATCGCTGGCTTC′] |
| 1963 | NM_000257.3(MYH7): c.2093T > | 4625 | MYH7 | [′ATGCACCAGCTGCGCTGCAATGGTGYGCTG |
| | C (p. Val698Ala) | | | GAGGGCATCCGCATCTGCAGG′] |
| 1964 | NM_000257.3(MYH7): c.2546T > | 4625 | MYH7 | [′AAGAGTGCAGAAAGAGAGAAGGAGAYGGC |
| | C (p.Met849Thr) | | | CTCCATGAAGGAGGAGTTCACA′] |
| 1965 | NM_000257.3(MYH7): c.2555T > | 4625 | MYH7 | [′GAAAGAGAGAAGGAGATGGCCTCCAHGAA |
| | C (p.Met852Thr) | | | GGAGGAGTTCACACGCCTCAAA′] |
| 1966 | NM_000257.3(MYH7): c.602T > | 4625 | MYH7 | [′CAGTACTTTGCTGTTATTGCAGCCAYTGGGG |
| | C (p.Ile201Thr) | | | ACCGCAGCAAGAAGGACCAG′] |
| 1967 | NM_000257.3(MYH7): c.788T > | 4625 | MYH7 | [′ACAGGAAAGTTGGCATCTGCAGACAYAGAG |
| | C (p.Ile263Thr) | | | ACCTGTGAGTGCCATGAATCT′] |
| 1968 | NM_000260.3(MYO7A): | 4647 | MYO7A | [′CTCCTGCCCCACGTGCAGCGCTTCCYGCAGT |
| | c.5573T > C (p.Leu1858Pro) | | | CCCGAAAGCACTGCCCACTC′] |
| 1969 | NM_000441.1(SLC26A4): | 5172 | SLC26A4 | [′GGAGAGCCTGGCCAAGTGCTGCAGGYAGCG |
| | c.164 + 2T > C | | | GCCGCGCGGGCCTGCGTAGAG′] |
| 1970 | NM_000441.1(SLC26A4): c.2T > | −1 | — | [′TCGCTGTCCTCTGGCTCGCAGGTCABGGCAG |
| | C (p.Met1Thr) | | | CGCCAGGCGGCAGGTCGGAG′] |
| 1971 | NM_000441.1(SLC26A4) | 5172 | SLC26A4 | [′AATGGAGTTCTCTCTATTATCTATGYAAGTG |
| | c.765 + 2T > C | | | TTGCTTCTTGCTCCAGGGAT′] |
| 1972 | NM_000551.3(VHL): c.497T > C | 7428 | VHL | [′CTGAAAGAGCGATGCCTCCAGGTTGYCCGG |
| | (p.Val166Ala) | | | AGCCTAGTCAAGCCTGAGAAT′] |
| 1973 | NM_001399.4(EDA): c.2T > C | 1896 | EDA | [′AGAGAGTGGGTGTCTCCGGAGGCCAYGGGC |
| | (p.Met1Thr) | | | TACCCGGAGGTGGAGCGCAGG′] |
| 1974 | NM_001943.3(DSG2): | 1829 | DSG2 | [′CTGTTGAAGAGTTGAGTGCAGCACGYAAGA |
| | c.523 + 2T > C | | | GTCTTTTTTTTTTTTTTTAAT′] |
| 1975 | NM_004572.3(PKP2): c.2062T > | 5318 | PKP2 | [′TTGCGGACACTTTTGGCGATCAAGGRCAGAT |
| | C (p.Ser688Pro) | | | ACATCCTTATAACAATGGAA′] |
| 1976 | NM_005633.3(SOS1): c.1310T > | 6654 | SOS1 | [′ATTGATGGTTGGGAGGGAAAAGACABTGGA |
| | C (p.Ile437Thr) | | | CAGTGTTGTAATGAATTTATA′] |
| 1977 | NM_170707.3(LMNA): c.799T > | 4000 | LMNA | [′GTATAAGAAGGAGCTGGAGAAGACTYATTC |
| | C (p.Tyr267His) | | | TGCCAAGGTGCTTGCTCTCGA′] |
| 1978 | NM_206933.2(USH2A): | 7399 | USH2A | [′TCTTGAAGATACAATTGTCTTAAACYGGAGA |
| | c.10561T > C (p.Trp3521Arg) | | | AAACCTATACAATCAAATGG′] |
| 1979 | NM_206933.2(USH2A): | 7399 | USH2A | [′CGACACAACAAGCCAGCCATATAGAYGCCT |
| | c.1606T > C (p.Cys536Arg) | | | CTGCTCCCAGGAGAGCTTCAC′] |
| 1980 | NM_206933.2(USH2A): | −1 | — | [′GTCGAGGACGTACAACAGGAGCAGGYAAAT |
| | c.5857 + 2T > C | | | ACTTATCTTCAAATGCATATG′] |
| 1981 | NM_000492.3(CFTR): c.1090T > | 1080 | CFTR | [′CTGGGCTGTACAAACATGGTATGACYCTCTT |
| | C (p.Ser364Pro) | | | GGAGCAATAAACAAAATACA′] |
| 1982 | NM_000548.3(TSC2): c.5150T > | 7249 | TSC2 | [′CCCTTCGTGGCCCGCCAGATGGCCCYGCACG |
| | C (p.Leu1717Pro) | | | CAAATGTGAGTGGGGGTGGG′] |
| 1983 | NM_000548.3(TSC2): c.2410T > | 7249 | TSC2 | [′CCTCATCCACCGCTGTGCCAGCCAGYGCGTC |
| | C (p.Cys804Arg) | | | GTGGCCTTGTCCATCTGCAG′] |
| 1984 | NM_000548.3(TSC2): c.3106T > | 7249 | TSC2 | [′GGACATGATGGCTCGATACGTCTTCYCCAAC |
| | C (p.Ser1036Pro) | | | TTCACGGCTGTCCCGAAGAG′] |
| 1985 | NM_000212.2(ITGB3): c.2231T > | −1 | — | [′GCCGCCCTGCTCATCTGGAAACTCCYCATCA |
| | C (p.Leu744Pro) | | | CCATCCACGACCGAAAAGAA′] |
| 1986 | NM_183415.2(UBE3B): | 89910 | UBE3B | [′AGATGATCTGGGATGGAATTGTAGGYAAGA |
| | c.1741 + 2T > C | | | GAAAAGGTGTCTGCTGTTGTT′] |
| 1987 | NM_005120.2(MED12): | 9968 | MED12 | [′CCTTTTAGCTTGTAGTGAACAGGACYCTGAG |
| | c.3493T > C (p.Ser1165Pro) | | | CCAGGGGCCCGGCTTACCTG′] |
| 1988 | NM_001033053.2(NLRP1): | 22861 | NLRP1 | [′CTAGCCCTCCATACCTGGGAGCAGAYGGGG |
| | c.230T > C (p.Met77Thr) | | | CTGAGGTCACTGTGCGCCCAA′] |
| 1989 | NM_025152.2(NUBPL): | 80224 | NUBPL | [′ATGCCTATATGAACTTTTCTGGTTCYAATGG |
| | c.815 − 27T > C | | | ATGTCTGCTGGGCTCTTTTA′] |
| 1990 | NM_001288953.1(TTC7A): | 57217 | TTC7A | [′GCGTGGCAGGGCCTGGGCGAGGTGCYGCAG |
| | c.2366T > C (p.Leu789Pro) | | | GCCCAGGGCCAGAACGAGGCT′] |
| 1991 | NM_014845.5(FIG4): c.524T > C | 9896 | FIG4 | [′TACAGCTATGATTTGTCCCACTCACYTCAAT |
| | (p.Leu175Pro) | | | ATAATCTCACTGTCTTGCGA′] |
| 1992 | NM_000059.3(BRCA2): c.2T > C | 675 | BRCA2 | [′ATTGGAGGAATATCGTAGGTAAAAABGCCT |
| | (p.Met1Thr) | | | ATTGGATCCAAAGAGAGGCCA′] |
| 1993 | NM_000059.3(BRCA2): c.316 + 2 | 675 | BRCA2 | [′TAGATAAATTCAAATTAGACTTAGGYAAGT |
| | T > C | | | AATGCAATATGGTAGACTGGG′] |
| 1994 | NM_000059.3(BRCA2): c.67 + 2T | 675 | BRCA2 | [′TTAAGACACGCTGCAACAAAGCAGGHATTG |
| | > C | | | ACAAATTTTATATAACTTTAT′] |
| 1995 | NM_000059.3(BRCA2): | 675 | BRCA2 | [′AGATGCCTAAGCCCAGAAAGGGTGCYTCTT |
| | c.7958T > C (p.Leu2653Pro) | | | CAACTAAAATACAGGCAAGTT′] |
| 1996 | NM_000218.2(KCNQ1): | 3784 | KCNQ1 | [′TGCTTCTCTGTCTTTGCCATCTCCTHCTTTGC |
| | c.1016T > C (p.Phe339Ser) | | | GCTCCCAGCGGTAGGTGCC′] |
| | NM_000218.2(KCNQ1): | 3784 | KCNQ1 | [] |
| | c.1045T > C (p.Ser349Pro) | | | |
| | NM_000218.2(KCNQ1): | 3784 | KCNQ1 | [] |
| | c.1052T > C (p.Phe351Ser) | | | |
| | NM_000218.2(KCNQ1): | 3784 | KCNQ1 | [] |
| | c.1058T> C (p.Leu353Pro) | | | |
| | NM_000218.2(KCNQ1): | 3784 | KCNQ1 | [] |
| | c.1541T > C (p.Ile514Thr) | | | |
| | NM_000218.2(KCNQ1): | 3784 | KCNQ1 | [] |
| | c.1661T > C (p.Val554Ala) | | | |
| 1997 | NM_000218.2(KCNQ1): | 3784 | KCNQ1 | [′CTTCTCTCCAGGCTGGACCAGTCCABTGGGA |
| | c.1700T > C (p.Ile567Thr) | | | AGCCCTCACTGTTCATCTCC′] |
| | NM_000218.2(KCNQ1): c.2T > C | 3784 | KCNQ1 | [] |
| | (p.Met1Thr) | | | |
| | NM_000218.2(KCNQ1): c.341T > | 3784 | KCNQ1 | [] |
| | C (p.Leul14Pro) | | | |
| 1998 | NM_000218.2(KCNQ1): c.560T > | 3784 | KCNQ1 | [′GGCTGCCGCAGCAAGTACGTGGGCCYCTGG |
| | C (p.Leu187Pro) | | | GGGCGGCTGCGCTTTGCCCGG′] |
| | NM_000218.2(KCNQ1): c.572T > | 3784 | KCNQ1 | [] |
| | C (p.Leu191Pro) | | | |
| | NM_000218.2(KCNQ1): c.716T > | 3784 | KCNQ1 | [] |
| | C (p.Leu239Pro) | | | |
| | NM_000218.2(KCNQ1): c.752T > | 3784 | KCNQ1 | [] |
| | C (p.Leu251Pro) | | | |
| | NM_000218.2(KCNQ1): c.797T > | 3784 | KCNQ1 | [] |
| | C (p.Leu266Pro) | | | |
| | NM_000218.2(KCNQ1): c.824T > | 3784 | KCNQ1 | [] |
| | C (p.Phe275Ser) | | | |
| | NM_000218.2(KCNQ1): c.910T > | 3784 | KCNQ1 | [] |
| | C (p.Trp304Arg) | | | |
| | NM_000218.2(KCNQ1): c.913T > | 3784 | KCNQ1 | [] |
| | C (p.Trp305Arg) | | | |
| 1999 | NM_000492.3(CFTR): c.1021T > | 1080 | CFTR | [′CCTCCGGAAAATATTCACCACCATCYCATTC |
| | C (p.Ser341Pro) | | | TGCATTGTTCTGCGCATGGC′] |
| 2000 | NM_000492.3(CFTR): c.1853T > | 1080 | CFTR | [′TTAAAGAAAGCTGACAAAATATTAAYTTTG |
| | C (p.Ile618Thr) | | | CATGAAGGTAGCAGCTATTTT′] |
| 2001 | NM_000492.3(CFTR): c.2780T > | 1080 | CFTR | [′TACGTGGGAGTAGCCGACACTTTGCYTGCTA |
| | C (p.Leu927Pro) | | | TGGGATTCTTCAGAGGTCTA′] |
| 2002 | NM_000492.3(CFTR): c.3230T > | 1080 | CFTR | [′GGACGGCAGCCTTACTTTGAAACTCYGTTCC |
| | C (p.Leu1077Pro) | | | ACAAAGCTCTGAATTTACAT′] |
| 2003 | NM_007294.3(BRCA1): c.115T > | 672 | BRCA1 | [′GATCAAGGAACCTGTCTCCACAAAGNGTGA |
| | C (p.Cys39Arg) | | | CCACATATTTTGCAAGTAAGT′] |
| 2004 | NM_007294.3(BRCA1): c.190T > | 672 | BRCA1 | [′GAAAGGGCCTTCACAGTGTCCTTTABGTAAG |
| | C (p.Cys64Arg) | | | AATGATATAACCAAAAGGTA′] |
| 2005 | NM_007294.3(BRCA1): | 672 | BRCA1 | [′ATGTAAGAATGATATAACCAAAAGGYATAT |
| | c.212 + 2T > C | | | AATTTGGTAATGATGCTAGGT′] |
| 2006 | NM_007294.3(BRCA1): c.2T > C | 672 | BRCA1 | [′TAAAGTTCATTGGAACAGAAAGAAABGGAT |
| | (p.Met1Thr) | | | TTATCTGCTCTTCGCGTTGAA′] |
| 2007 | NM_007294.3(BRCA1): | 672 | BRCA1 | [′CAGAACAAAGCACATCAGAAAAAGGBGTGT |
| | c.4357 + 2T > C | | | ATTGTTGGCCAAACACTGATA′] |
| 2008 | NM_007294.3(BRCA1): | 672 | BRCA1 | [′CTCTTCTTCCAGATCTTCAGGGGGCYAGAAA |
| | c.5291T > C (p.Leu1764Pro) | | | TCTGTTGCTATGGGCCCTTC′] |
| 2009 | NM_007294.3(BRCA1): c.65T > C | 672 | BRCA1 | [′GTCATTAATGCTATGCAGAAAATCTYAGAGT |
| | (p.Leu22Ser) | | | GTCCCATCTGGTAAGTCAGC′] |
| 2010 | NM_005236.2(ERCC4): c.689T > | 2072 | ERCC4 | [′ATGCTTGCTATACAGACTGCTATACYGGACA |
| | C (p.Leu230Pro) | | | TTTTAAATGCATGTCTAAAG′] |
| 2011 | NM_005236.2(ERCC4): c.706T > | 2072 | ERCC4 | [′TGCTATACTGGACATTTTAAATGCAYGTCTA |
| | C (p.Cys236Arg) | | | AAGGAACTAAAATGCCATAA′] |
| 2012 | NM_000435.2(NOTCH3): | 4854 | NOTCH3 | [′GGCGTGCTGGTGCTCACAGTGCTGCYGCCGC |
| | c.4556T > C (p.Leu1519Pro) | | | CAGAGGAGCTACTGCGTTCC′] |
| 2013 | NM_153704.5(TMEM67): | 91147 | TMEM67 | [′GGAAATTTTCTCAAGTGGCAAACTTYAGAA |
| | c.1046T > C (p.Leu349Ser) | | | GGAGGTGTTTTACAGGTAAGC′] |
| 2014 | NM_001142519.1(FAM111A): | 63901 | FAM111A | [′GTCTAAAAAAGCAGAAAGTCCAGAGYATGT |
| | c.1531T > C (p.Tyr511His) | | | CCATATGTATACTCAAAGAAG′] |
| 2015 | NM_014795.3(ZEB2): c.3211T > | 9839 | ZEB2 | [′GCGCTTCTCACACTCGGGCTCGTACYCGCAG |
| | C (p.Ser1071Pro) | | | CACATGAATCACAGGTATTC′] |
| 2016 | NM_001199107.1(TBC1D24): | 57465 | TBC1D24 | [′CCCCTCTGCTACTTCGCCCGGGTCTYTGACG |
| | c.686T > C (p.Phe229Ser) | | | TCTTCCTGGTGGAGGGCTAC′] |
| 2017 | NM_022114.3(PRDM16): | 63976 | PRDM16 | [′GTGGGAGCCCTGAAGGAGAAGTACCYGCGG |
| | c.2660T > C (p.Leu887Pro) | | | CCGTCCCCGCTGCTCTTCCAC′] |
| 2018 | NM_001130089.1(KARS): | 3735 | KARS | [′AGCTTCTGGGGGAAAGCTCATCTTCYATGAT |
| | c.517T > C (p.Tyr173His) | | | CTTCGAGGAGAGGGGGTGAA′] |
| 2019 | NM_005689.2(ABCB6): | 10058 | ABCB6 | [′CTCATCTTCTCCCACCTGCACGAGCYCTCAC |
| | c.1067T > C (p.Leu356Pro) | | | TGCGCTGGCACCTGGGGCGC′] |
| 2020 | NM_001070.4(TUBG1): | 7283 | TUBG1 | [′CCCCTGTTTTCTGCACACCCCAAGCYCTTCG |
| | c.1160T > C (p.Leu387Pro) | | | AGAGAACCTGTCGCCAGTAT′] |
| 2021 | NM_001283009.1(RTEL1): | −1 | — | [′CGGGCCCCTCTCAGCAGGCTGTGTGYGCCA |
| | c.3730T > C (p.Cys1244Arg) | | | GGGCTGTGGGGCAGAGGACGT′] |
| 2022 | NM_001135021.1(ELMOD3): | 84173 | ELMOD3 | [′ATCACCCACATTGCCATCCAGGCCTYGAGA |
| | c.794T > C (p.Leu265Ser) | | | GAGGAGTGTCTCTCCAGGTGA′] |
| 2023 | NM_001382.3(DPAGT1): | 1798 | DPAGT1 | [′CTACCATCTCTCCCCGCAGGAATCCYGTACT |
| | c.503T> C (p.Leu168Pro) | | | ATGTCTACATGGGGCTGCTG′] |
| 2024 | m.10237T > C | 4537 | MT-ND3 | [′CCATAAAATTCTTCTTAGTAGCTATYACCTT |
| | | | | CTTATTATTTGATCTAGAAA′] |
| 2025 | m.11253T > C | 4538 | MT-ND4 | [′TCCCTTCCCCTACTCATCGCACTAAYTTACA |
| | | | | CTCACAACACCCTAGGCTCA′] |
| 2026 | m.12811T > C | 4540 | MT-ND5 | [′ATCCTTCTTGCTCATCAGTTGATGAYACGCC |
| | | | | CGAGCAGATGCCAACACAGC′] |
| 2027 | m.14325T > C | 4541 | MT-ND6 | [′ATTCAGCTTCCTACACTATTAAAGTYTACCA |
| | | | | CAACCACCACCCCATCATAC′] |
| 2028 | NM_000142.4(FGFR3): | 2261 | FGFR3 | [′ACCCAGCAGTGGGGGCTCGCGGACGNGAAG |
| | c.2419T > C (p.Ter807Arg) | | | GGCCACTGGTCCCCAACAATG′] |
| 2029 | NM_000370.3(TTPA): c.548T > C | 7274 | TTPA | [′GTAGCCAAGAAGATTGCTGCTGTACYTACG |
| | (p.Leu183Pro) | | | GTAAATGTATATTTTAACTGT′] |
| 2030 | NM_000375.2(UROS): c.139T > C | 7390 | UROS | [′TGAGTTTTTGTCTCTTCCCAGTTTCYCTGAGA |
| | (p.Ser47Pro) | | | AGGTAAGGCCTGTTGTGAC′] |
| 2031 | NM_001006657.1(WDR35): | 57539 | WDR35 | [′TCTGGCACCATTCAGAGATACAGTCYACCTA |
| | c.1592T > C (p.Leu531Pro) | | | ATGTTGGTTTGATTCAAAAA′] |
| 2032 | NM_001876.3(CPT1A): | 1374 | CPT1A | [′GCAGATGCGCCGATCGTGGCCCACCYTTGG |
| | c.1451T > C (p.Leu484Pro) | | | GAGGTGAGTTTTCACACTTTT′] |
| 2033 | NM_004595.4(SMS): c.449T > C | 6611 | SMS | [′GACGAAGATTCACCTTATCAAAATAYAAAA |
| | (p.Ile150Thr) | | | ATTCTACACTCGAAGCAGTTT′] |
| 2034 | NM_005211.3(CSF1R): c.2483T > | 1436 | CSF1R | [′AAGTGGATGGCCCCAGAGAGCATCTYTGAC |
| | C (p.Phe828Ser) | | | TGTGTCTACACGGTTCAGAGC′] |
| 2035 | NM_170707.3(LMNA): c.644T > | 4000 | LMNA | [′TCCTTCCTCCAACCCTTCCAGGAGCYGCGTG |
| | C (p.Leu215Pro) | | | AGACCAAGCGCCGTCATGAG′] |
| 2036 | NM_194248.2(OTOF): c.3413T > | 9381 | OTOF | [′CCACCCTCCAACCTCTCCCAGGTGCYGTTCT |
| | C (p.Leu1138Pro) | | | GGGGCCTACGGGACCTAAAG′] |
| 2037 | NM_000540.2(RYR1): c.14762 | 6261 | RYR1 | [′TACAGGGTGGTCTTCGACATCACCTYCTTCT |
| | > C (p.Phe4921Ser) | | | TCTTCGTCATCGTCATCCTG′] |
| 2038 | NM_000540.2(RYR1): c.7358T > | 6261 | RYR1 | [′GCCGGCAAGGGTGAGGCCCTGCGGAYCCGC |
| | C (p.Ile2453Thr) | | | GCCATCCTCCGCTCCCTTGTG′] |
| 2039 | NM_000540.2(RYR1): | 6261 | RYR1 | [′GAAGTGTCAGCCGTGCTCTACTACCYGGACC |
| | c.10817T> C (p.Leu3606Pro) | | | AGGTGGGTGGGGCCGGAGGG′] |
| 2040 | NM_000540.2(RYR1): | 6261 | RYR1 | [′TTTTACACCCTGCGGTTCCTTGCCCYCTTCTT |
| | c.13703T > C (p.Leu4568Pro) | | | GGCATTTGCCATCAACTTC′] |
| 2041 | NM_000540.2(RYR1): | 6261 | RYR1 | [′CCCGCCCTGCGGTGTCTGAGCCTCCYGCATA |
| | c. 13949T > C (p.Leu4650Pro) | | | CACTGGTGGCCTTTCTCTGC′] |
| 2042 | NM_000540.2(RYR1): | 6261 | RYR1 | [′GTGTGCCCACAGTCCTTCCTGTACCYGGGCT |
| | c.14378T > C (p.Leu4793Pro) | | | GGTATATGGTGATGTCCCTC′] |
| 2043 | NM_015896.3(ZMYND10): | 51364 | ZMYND10 | [′CTCAGGGCTTAGCAGCAGGTTGTACRGGGC |
| | 7c.79T > C (p.Leu266Pro) | | | GATCCACACTTGCCCGTCCAA′] |
| 2044 | NM_018127.6(ELAC2): c.460T > | 60528 | ELAC2 | [′AAAATACCTCGAAGCAATCAAAATAYTTTCT |
| | C (p.Phe154Leu) | | | GGTCCATTGAAAGGAATAGA′] |
| 2045 | NM_199355.2(ADAMTS18): | 170692 | ADAMTS18 | [′CCTGCGGGTCACCATCCTCACGTACYGTACA |
| | c.605T > C (p.Leu202Pro) | | | AAAGGACAGCAGAGGAGAAG′] |
| 2046 | NM_023110.2(FGFR1): c.494T > | 2260 | FGFR1 | [′TCCCCAGAAAAGATGGAAAAGAAATYGCAT |
| | C (p.Leu165Ser) | | | GCAGTGCCGGCTGCCAAGACA′] |
| 2047 | NM_001059.2(TACR3): c.766T > | 6870 | TACR3 | [′CCATATTATCGTCATTATACTGGTGYACTGT |
| | C (p.Tyr256His) | | | TTCCCATTGCTCATCATGGG′] |
| 2048 | NM_002055.4(GFAP): c.1070T > | 2670 | GFAP | [′TACCAGGACCTGCTCAATGTCAAGCHGGCC |
| | C (p.Leu357Pro) | | | CTGGACATCGAGATCGCCACC′] |
| 2049 | NM_002055.4(GFAP): c.1076T > | 2670 | GFAP | [′GACCTGCTCAATGTCAAGCTGGCCCYGGAC |
| | C (p.Leu359Pro) | | | ATCGAGATCGCCACCTACAGG′] |
| 2050 | NM_002055.4(GFAP): c. 1096T > | 2670 | GFAP | [′GGCCCTGGACATCGAGATCGCCACCYACAG |
| | C (p.Tyr366His) | | | GAAGCTGCTAGAGGGCGAGGA′] |
| 2051 | NM_002055.4(GFAP): c.221T > C | 2670 | GFAP | [′CGGGCCAGTGAGCGGGCAGAGATGAYGGAG |
| | (p.Met74Thr) | | | CTCAATGACCGCTTTGCCAGC′] |
| 2052 | NM_002055.4(GFAP): c.247T > C | 2670 | GFAP | [′GGAGCTCAATGACCGCTTTGCCAGCYACATC |
| | (p.Tyr83His) | | | GAGAAGGTTCGCTTCCTGGA′] |
| 2053 | NM_002055.4(GFAP): c.269T > C | 2670 | GFAP | [′AGCTACATCGAGAAGGTTCGCTTCCYGGAA |
| | (p.Leu90Pro) | | | CAGCAAAACAAGGCGCTGGCT′] |
| 2054 | NM_002055.4(GFAP): c.290T > C | 2670 | GFAP | [′TTCCTGGAACAGCAAAACAAGGCGCYGGCT |
| | (p.Leu97Pro) | | | GCTGAGCTGAACCAGCTGCGG′] |
| 2055 | NM_002055.4(GFAP): c.302T > C | 2670 | GFAP | [′CAAAACAAGGCGCTGGCTGCTGAGCYGAAC |
| | (p.Leu101Pro) | | | CAGCTGCGGGCCAAGGAGCCC′] |
| 2056 | NM_002055.4(GFAP): c.704T > C | 2670 | GFAP | [′GCCAAGCCAGACCTCACCGCAGCCCYGAAA |
| | (p.Leu235Pro) | | | GAGATCCGCACGCAGTATGAG′] |
| 2057 | NM_002055.4(GFAP): c.739T > C | 2670 | GFAP | [′CCGCACGCAGTATGAGGCAATGGCGYCCAG |
| | (p.Ser247Pro) | | | CAACATGCATGAAGCCGAAGA′] |
| 2058 | NM_002055.4(GFAP): c.992T > C | 2670 | GFAP | [′AGTTATCAGGAGGCGCTGGCGCGGCYGGAG |
| | (p.Leu331Pro) | | | GAAGAGGGGCAGAGCCTCAAG′] |
| 2059 | NM_005554.3(KRT6A): | 3853 | KRT6A | [′GAGATCGCCACCTACCGCAAGCTGCBGGAG |
| | c.1406T > C (p.Leu469Pro) | | | GGTGAGGAGTGCAGGTGGGTA′] |
| 2060 | NM_005554.3(KRT6A): c.521T > | 3853 | KRT6A | [′CAGATCAAGACCCTCAACAACAAGTBTGCC |
| | C (p.Phe174Ser) | | | TCCTTCATCGACAAGGTGAGC′] |
| 2061 | NM_153490.2(KRT13): c.332T > | 3860 | KRT13 | [′AATGAGAAGATCACCATGCAGAACCYCAAC |
| | C (p.Leu111Pro) | | | GACCGCCTGGCTTCCTACCTG′] |
| 2062 | NM_170707.3(LMNA): c.1619T > | 4000 | LMNA | [′CCATGTCCCCACCAGGAAGTGGCCAYGCGC |
| | C (p.Met540Thr) | | | AAGCTGGTGCGCTCAGTGACT′] |
| | NM_000218.2(KCNQ1): | 3784 | KCNQ1 | [] |
| | c.1117T > C (p.Ser373Pro) | | | |
| 2063 | NM_000218.2(KCNQ1): | 3784 | KCNQ1 | [′TCCCCCTGCCCGACCTCAGACCGCABGGAG |
| | c.1135T > C (p.Trp379Arg) | | | GTGCTATGCTGCCGAGAACCC′] |
| | NM_000218.2(KCNQ1): | 3784 | KCNQ1 | [] |
| | c.1165T> C (p.Ser389Pro) | | | |
| | NM_000218.2(KCNQ1): | 3784 | KCNQ1 | [] |
| | c.1174T > C (p.Trp392Arg) | | | |
| | NM_000218.2(KCNQ1): | 3784 | KCNQ1 | [] |
| | c.1550T > C (p.Ile517Thr) | | | |
| | NM_000218.2(KCNQ1): | 3784 | KCNQ1 | [] |
| | c.1696T > C (p.Ser566Pro) | | | |
| | NM_000218.2(KCNQ1): | −1 | — | [] |
| | c.1805T > C (p.Leu602Pro) | | | |
| | NM_000218.2(KCNQ1): c.401T > | 3784 | KCNQ1 | [] |
| | C (p.Leu134Pro) | | | |
| 2064 | NM_000218.2(KCNQ1): c.550T > | 3784 | KCNQ1 | [′CTGGTCCGCCGGCTGCCGCAGCAAGBACGT |
| | C (p.Tyr184His) | | | GGGCCTCTGGGGGCGGCTGCG′] |
| | NM_000218.2(KCNQ1): c.608T > | 3784 | KCNQ1 | [] |
| | C (p.Leu203Pro) | | | |
| | NM_000218.2(KCNQ1): c.625T > | 3784 | KCNQ1 | [] |
| | C (p.Ser209Pro) | | | |
| | NM_000218.2(KCNQ1): c.742T > | 3784 | KCNQ1 | [] |
| | C (p.Trp248Arg) | | | |
| 2065 | NM_000218.2(KCNQ1): c.749T > | 3784 | KCNQ1 | [′GACCGCCAGGGAGGCACCTGGAGGCHCCTG |
| | C (p.Leu250Pro) | | | GGCTCCGTGGTCTTCATCCAC′] |
| | NM_000218.2(KCNQ1): c.829T > | 3784 | KCNQ1 | [] |
| | C (p.Ser277Pro) | | | |
| | NM_000218.2(KCNQ1): c.832T > | 3784 | KCNQ1 | [] |
| | C (p.Tyr278His) | | | |
| | NM_000218.2(KCNQ1): c.845T > | 3784 | KCNQ1 | [] |
| | C (p.Leu282Pro) | | | |
| | NM_000218.2(KCNQ1): c.908T > | 3784 | KCNQ1 | [] |
| | C (p.Leu303Pro) | | | |
| 2066 | NM_000238.3(KCNH2): c.122T > | 3757 | KCNH2 | [′AACGCTCGGGTGGAGAACTGCGCCGBCATC |
| | C (p. Val41Ala) | | | TACTGCAACGACGGCTTCTGC′] |
| | NM_000238.3(KCNH2): | 3757 | KCNH2 | [] |
| | c.1238T > C (p.Leu413Pro) | | | |
| | NM_000238.3(KCNH2): | 3757 | KCNH2 | [] |
| | c.1279T > C (p.Tyr427His) | | | |
| | NM_000238.3(KCNH2): | 3757 | KCNH2 | [] |
| | c.1387T > C (p.Phe463Leu) | | | |
| | NM_000238.3(KCNH2): c.160T > | 3757 | KCNH2 | [] |
| | C (p.Tyr54His) | | | |
| 2067 | NM_000238.3(KCNH2): | 3757 | KCNH2 | [′GAGTACGGCGCGGCCGTGCTGTTCTYGCTCA |
| | c.1655T > C (p.Leu552Ser) | | | TGTGCACCTTTGCGCTCATC′] |
| | NM_000238.3(KCNH2): | 3757 | KCNH2 | [] |
| | c.1691T > C (p.Leu564Pro) | | | |
| | NM_000238.3(KCNH2): | 3757 | KCNH2 | [] |
| | c.1700T > C (p.Ile567Thr) | | | |
| | NM_000238.3(KCNH2): | 3757 | KCNH2 | [] |
| | c.1702T > C (p.Trp568Arg) | | | |
| | NM_000238.3(KCNH2): | 3757 | KCNH2 | [] |
| | c.1705T > C (p.Tyr569His) | | | |
| | NM_000238.3(KCNH2): | 3757 | KCNH2 | [] |
| | c.1736T > C (p.Met579Thr) | | | |
| 2068 | NM_000238.3(KCNH2): | 3757 | KCNH2 | [′TGGCTGCACAACCTGGGCGACCAGANAGGC |
| | c.1778T > C (p.Ile593Thr) | | | AAACCCTACAACAGCAGCGGC′] |
| | NM_000238.3(KCNH2): | 3757 | KCNH2 | [] |
| | c.1816T > C (p.Ser606Pro) | | | |
| 2069 | NM_000238.3(KCNH2): | 3757 | KCNH2 | [′CTTCAGCAGCCTCACCAGTGTGGGCHTCGGC |
| | c.1879T > C (p.Phe627Leu) | | | AACGTCTCTCCCAACACCAA′] |
| | NM_000238.3(KCNH2): | 3757 | KCNH2 | [] |
| | c.1889T > C (p.Val630Ala) | | | |
| | NM_000238.3(KCNH2): | 3757 | KCNH2 | [] |
| | c.1945T > C (p.Ser649Pro) | | | |
| | NM_000238.3(KCNH2): | 3757 | KCNH2 | [] |
| | c.1985T > C (p.Ile662Thr) | | | |
| | NM_000238.3(KCNH2): c.202T > | 3757 | KCNH2 | [] |
| | C (p.Phe68Leu) | | | |
| | NM_000238.3(KCNH2): | 3757 | KCNH2 | [] |
| | c.2033T > C (p.Leu678Pro) | | | |
| | NM_000238.3(KCNH2): c.206T > | 3757 | KCNH2 | [] |
| | C (p.Leu69Pro) | | | |
| | NM_000238.3(KCNH2): | 3757 | KCNH2 | [] |
| | c.2078T > C (p.Leu693Pro) | | | |
| | NM_000238.3(KCNH2): | 3757 | KCNH2 | [] |
| | c.2309T > C (p.Val770Ala) | | | |
| 2070 | NM_000238.3(KCNH2): | 3757 | KCNH2 | [′TGGCCTCCAGGGAAGAATGACATCTBTGGG |
| | c.2414T > C (p.Phe805Ser) | | | GAGCCTCTGAACCTGTATGCA′] |
| | NM_000238.3(KCNH2): | 3757 | KCNH2 | [] |
| | c.2452T > C (p.Ser818Pro) | | | |
| | NM_000238.3(KCNH2): | 3757 | KCNH2 | [] |
| | c.2573T > C (p.Ile858Thr) | | | |
| 2071 | NM_000238.3(KCNH2): c.257T > | 3757 | KCNH2 | [′GCTGCCGCGCAGATCGCGCAGGCACBGCTG |
| | C (p.Leu86Pro) | | | GGCGCCGAGGAGCGCAAAGTG′] |
| | NM_000238.3(KCNH2): c.260T > | 3757 | KCNH2 | [] |
| | C (p.Leu87Pro) | | | |
| | NM_000238.3(KCNH2): c.287T > | 3757 | KCNH2 | [] |
| | C (p.Ile96Thr) | | | |
| | NM_000238.3(KCNH2): | 3757 | KCNH2 | [] |
| | c.3146T > C (p.Leu1049Pro) | | | |
| | NM_000238.3(KCNH2): c.322T > | 3757 | KCNH2 | [] |
| | C (p.Cys108Arg) | | | |
| 2072 | NM_000238.3(KCNH2): c.371T > | 3757 | KCNH2 | [′AAGAACGAGGATGGGGCTGTCATCABGTTC |
| | C (p.Met124Thr) | | | ATCCTCAATTTCGAGGTGGTG′] |
| | NM_000238.3(KCNH2): c.65T > | 3757 | KCNH2 | [] |
| | C (p.Phe22Ser) | | | |
| | NM_000238.3(KCNH2): c.86T > | 3757 | KCNH2 | [] |
| | C (p.Phe29Ser) | | | |
| | NM_000238.3(KCNH2): c.872T > | 3757 | KCNH2 | [] |
| | C (p.Met291Thr) | | | |
| | NM_000238.3(KCNH2): c.89T > | 3757 | KCNH2 | [] |
| | C (p.Ile30Thr) | | | |
| 2073 | NM_000238.3(KCNH2): c.92T > | 3757 | KCNH2 | [′GCCCCCCTAGGCCGTAAGTTCATCABCGCCA |
| | C (p.Ile31Thr) | | | ACGCTCGGGTGGAGAACTGC′] |
| | NM_000891.2(KCNJ2): c.301T > | 3759 | KCNJ2 | [] |
| | C (p.Cys101Arg) | | | |
| | NM_000891.2(KCNJ2): c.650T > | 3759 | KCNJ2 | [] |
| | C (p.Leu217Pro) | | | |
| | NM_000335.4(SCN5A): | 6331 | SCN5A | [] |
| | c.1187T > C (p. Val396Ala) | | | |
| | NM_000335.4(SCN5A): | 6331 | SCN5A | [] |
| | c.1190T > C (p.Ile397Thr) | | | |
| | NM_000335.4(SCN5A): | 6331 | SCN5A | [] |
| | c.2018T > C (p.Leu673Pro) | | | |
| | NM_000335.4(SCN5A): | 6331 | SCN5A | [] |
| | c.2516T > C (p.Leu839Pro) | | | |
| | NM_000335.4(SCN5A): | 6331 | SCN5A | [] |
| | c.2551T > C (p.Phe851Leu) | | | |
| | NM_000335.4(SCN5A): | 6331 | SCN5A | [] |
| | c.2743T > C (p.Cys915Arg) | | | |
| | NM_000335.4(SCN5A): | 6331 | SCN5A | [] |
| | c.2783T > C (p.Leu928Pro) | | | |
| | NM_000335.4(SCN5A): c.278T > | 6331 | SCN5A | [] |
| | C (p.Phe93Ser) | | | |
| | NM_000335.4(SCN5A): | 6331 | SCN5A | [] |
| | c.2804T > C (p.Leu935Pro) | | | |
| 2074 | NM_000335.4(SCN5A): | 6331 | SCN5A | [′CAAGCGGACCACCTGGGATTTCTGCYGTGGT |
| | c.2944T > C (p.Cys982Arg) | | | CTCCTGCGGCAGCGGCCTCA′] |
| 2075 | NM_198056.2(SCN5A): | 6331 | SCN5A | [′TGCCGCCCAGGGCCAGCTGCCCAGCYGCAT |
| | c.3010T > C (p.Cys1004Arg) | | | TGCCACCCCCTACTCCCCGCC′] |
| 2076 | NM_000335.4(SCN5A): | 6331 | SCN5A | [′TTGTCTGCAGGCCTTCGAGGACATCYACCTA |
| | c.3679T > C (p.Tyr1227His) | | | GAGGAGCGGAAGACCATCAA′] |
| | NM_000335.4(SCN5A): | 6331 | SCN5A | [] |
| | c.3713T > C (p.Leu1238Pro) | | | |
| 2077 | NM_000335.4(SCN5A): | 6331 | SCN5A | [′TGCCGACAAGATGTTCACATATGTCYTCGTG |
| | c.3745T > C (p.Phe1249Leu) | | | CTGGAGATGCTGCTCAAGTG′] |
| | NM_000335.4(SCN5A): | 6331 | SCN5A | [] |
| | c.3929T > C (p.Leu1310Pro) | | | |
| | NM_000335.4(SCN5A): | 6331 | SCN5A | [] |
| | c.4027T > C (p.Phe1343Leu) | | | |
| | NM_000335.4(SCN5A): | 6331 | SCN5A | [] |
| | c.4028T > C (p.Phe1343Ser) | | | |
| | NM_000335.4(SCN5A): | 6331 | SCN5A | [] |
| | c.4034T > C (p.Leu1345Pro) | | | |
| | NM_000335.4(SCN5A): | 6331 | SCN5A | [] |
| | c.4046T > C (p.Ile1349Thr) | | | |
| | NM_000335.4(SCN5A): c.407T > | 6331 | SCN5A | [] |
| | C (p.Leul36Pro) | | | |
| | NM_000335.4(SCN5A): | 6331 | SCN5A | [] |
| | c.4340T > C (p.Ile1447Thr) | | | |
| 2078 | NM_000335.4(SCN5A): | 6331 | SCN5A | [′TTTATTGGTGTCATCATTGACAACTBCAACC |
| | c.4415T > C (p.Phe1472Ser) | | | AACAGAAGAAAAAGATACGT′] |
| | NM_000335.4(SCN5A): | 6331 | SCN5A | [] |
| | c.4453T> C (p.Phe1485Leu) | | | |
| | NM_198056.2(SCN5A): | 6331 | SCN5A | [] |
| | c.4493T > C (p.Met1498Thr) | | | |
| | NM_000335.4(SCN5A): | 6331 | SCN5A | [] |
| | c.4742T> C (p.Leu1581Pro) | | | |
| | NM_000335.4(SCN5A): | 6331 | SCN5A | [] |
| | c.4778T > C (p.Phe1593Ser) | | | |
| 2079 | NM_000335.4(SCN5A): | 6331 | SCN5A | [′CGCACGCTGCTCTTTGCCCTCATGABGTCCC |
| | c.4952T > C (p.Met1651Thr) | | | TGCCTGCCCTCTTCAACATC′] |
| | NM_000335.4(SCN5A): | 6331 | SCN5A | [] |
| | c.5111T > C (p.Phe1704Ser) | | | |
| | NM_000335.4(SCN5A): | 6331 | SCN5A | [] |
| | c.5179T > C (p.Cys1727Arg) | | | |
| | NM_000335.4(SCN5A): c.544T > | 6331 | SCN5A | [] |
| | C (p.Cys182Arg) | | | |
| 2080 | NM_000335.4(SCN5A): c.5471T | 6331 | SCN5A | [′TTTGCCGATGCCCTGTCTGAGCCACYCCGTA |
| | > C (p.Leu1824Pro) | | | TCGCCAAGCCCAACCAGATA′] |
| 2081 | NM_198056.2(SCN5A): | 6331 | SCN5A | [′GAGATGGACGCCCTGAAGATCCAGAHGGAG |
| | c.5624T > C (p.Met1875Thr) | | | GAGAAGTTCATGGCAGCCAAC′] |
| 2082 | NM_000335.4(SCN5A): c.635T > | 6331 | SCN5A | [′AGATACACAACTGAATTTGTGGACCHGGGC |
| | C (p.Leu212Pro) | | | AATGTCTCAGCCTTACGCACC′] |
| | NM_000335.4(SCN5A): c.689T > | 6331 | SCN5A | [] |
| | C (p.Ile230Thr) | | | |
| | NM_198056.2(SCN5A): c.944T > | 6331 | SCN5A | [] |
| | C (p.Leu315Pro) | | | |
| 2083 | NM_177976.2(ARL6): c.272T > C | 84100 | ARL6 | [′TTACACAGAGAAGGCCAAGCTATTAYTTTTG |
| | (p.Ile91Thr) | | | TCATTGATAGTAGTGATAGA′] |
| 2084 | NM_000487.5(ARSA): c.899T > C | 410 | ARSA | [′TCCCGAGGCGGCTGCTCCGGTCTCTYGCGGT |
| | (p.Leu300Ser) | | | GTGGAAAGGGAACGACCTAC′] |
| 2085 | NM_000267.3(NF1): c.1595T > C | 4763 | NF1 | [′GAATTAATTACAGGGCTCGTCCAACYGGTCC |
| | (p.Leu532Pro) | | | CTCAGTCACACATGCCAGAG′] |
| 2086 | NM_001042492.2(NF1): | 4763 | NF1 | [′CTTCAGAAAAGAGTGATGGCACTGCBGAGG |
| | c.2288T > C (p.Leu763Pro) | | | CGCATTGAGCATCCCACTGCA′] |
| 2087 | NM_004990.3(MARS): c.1108T > | 4141 | MARS | [′CCTCACCAGAATCACCCAGGACATTYTCCAG |
| | C (p.Phe370Leu) | | | CAGTTGCTGAAACGAGGTTT′] |
| 2088 | NM_004990.3(MARS): c.1568T > | 4141 | MARS | [′TTCTATGTCTGGTTTGATGCCACTAYTGGCT |
| | C (p.Ile523Thr) | | | ATCTGTCCATCACAGCCAAC′] |
| 2089 | NM_006920.4(SCN1A): | −1 | — | [′GTTCATTGTGCTATTTACTGGAGAGYGTGTA |
| | c.4729T > C (p.Cys1577Arg) | | | CTGAAACTCATCTCTCTACG′] |
| 2090 | NM_006920.4(SCN1A): | −1 | — | [′TGAGACCTTTGGCAACAGCATGATCYGCCTA |
| | c.5113T > C (p.Cys1705Arg) | | | TTCCAAATTACAACCTCTGC′] |
| 2091 | NM_006920.4(SCN1A): c.838T > | 6323 | SCN1A | [′CAACCTGAGGAATAAATGTATACAAYGGCC |
| | C (p.Trp280Arg) | | | TCCCACCAATGCTTCCTTGGA′] |
| 2092 | NM_006920.4(SCN1A): c.269T > | 6323 | SCN1A | [′TGTTGTGTTCCTGTCTTACAGACTTYTATAGT |
| | C (p.Phe90Ser) | | | ATTGAATAAAGGGAAGGCC′] |
| 2093 | NM_006920.4(SCN1A): c.272T > | 6323 | SCN1A | [′TGTGTTCCTGTCTTACAGACTTTTAYAGTATT |
| | C (p.Ile91Thr) | | | GAATAAAGGGAAGGCCATC′] |
| 2094 | NM_006920.4(SCN1A): | −1 | — | [′TATTTCACCAATGCCTGGTGTTGGCYGGACT |
| | c.3827T > C (p.Leu1276Pro) | | | TCTTAATTGTTGATGTAGGT′] |
| 2095 | NM_006920.4(SCN1A): | −1 | — | [′CCAAACAAACTCCAGCTCATTGCCAYGGATT |
| | c.5522T > C (p.Met1841Thr) | | | TGCCCATGGTGAGTGGTGAC′] |
| 2096 | NM_006920.4(SCN1A): c.568T > | 6323 | SCN1A | [′AGATTTTACTTTCCTTCGGGATCCAYGGAAC |
| | C (p.Trp190Arg) | | | TGGCTCGATTTCACTGTCAT′] |
| 2097 | NM_002608.2(PDGFB): c.356T > | 5155 | PDGFB | [′ATAGACCGCACCAACGCCAACTTCCYGGTG |
| | C (p.Leu119Pro) | | | TGGCCGCCCTGTGTGGAGGTG′] |
| 2098 | NM_000833.4(GRIN2A): c.2T > C | 2903 | GRIN2A | [′TTGCAGGGACCGTCAGTGGCGACTAYGGGC |
| | (p.Met1Thr) | | | AGAGTGGGCTATTGGACCCTG′] |
| 2099 | NM_002474.2(MYH11): | 4629 | MYH11 | [′CTCCCCATCGCTGCACTTGGACTGCDGCTCC |
| | c.3791T > C (p.Leu1264Pro) | | | TGCACCTGCGCCTCCAGCTT′] |
| 2100 | NM_014139.2(SCN11A): | 11280 | SCN11A | [′CTCAACCTCTTCATTGCCTTACTGCYCAATT |
| | c.2432T > C (p.Leu811Pro) | | | CCTTTAGCAATGAGGAAAGA′] |
| 2101 | NM_006514.3(SCN10A): | 6336 | SCN10A | [′GGAGTCAGGGTTGCTGGGTTGAGGARGAGG |
| | c.1661T > C (p.Leu554Pro) | | | GCTTCTAGGGAGGGGGCCTTG′] |
| 2102 | NM_000179.2(MSH6): c.1346T > | 2956 | MSH6 | [′CTTATTGGAGTCAGTGAACTGGGGCYGGTAT |
| | C (p.Leu449Pro) | | | TCATGAAAGGCAACTGGGCC′] |
| 2103 | NM_000179.2(MSH6): | 2956 | MSH6 | [′GAATCAGTCACTACGATTATTTCGGYAACTA |
| | c.4001 + 2T > C | | | ACTAACTATAATGGAATTAT′] |
| 2104 | NM_000249.3(MLH1): c.1745T > | 4292 | MLH1 | [′GCTTCTTCCTAGGAGCCAGCACCGCYCTTTG |
| | C (p.Leu582Pro) | | | ACCTTGCCATGCTTGCCTTA′] |
| 2105 | NM_000249.3(MLH1): c.2246T > | 4292 | MLH1 | [′CTGCAGCTTGCTAACCTGCCTGATCHATACA |
| | C (p.Leu749Pro) | | | AAGTCTTTGAGAGGTGTTAA′] |
| 2106 | NM_000249.3(MLH1): c.229T > C | 4292 | MLH1 | [′ACAGAAAGAAGATCTGGATATTGTAYGTGA |
| | (p.Cys77Arg) | | | AAGGTTCACTACTAGTAAACT′] |
| 2107 | NM_000249.3(MLH1): | 4292 | MLH1 | [′GGCAATCAAGGGACCCAGATCACGGYAAGA |
| | c.453 + 2T > C | | | ATGGTACATGGGAGAGTAAAT′] |
| 2108 | NM_000249.3(MLH1): c.739T > C | 4292 | MLH1 | [′AGCCTTCAAAATGAATGGTTACATABCCAAT |
| | (p.Ser247Pro) | | | GCAAACTACTCAGTGAAGAA′] |
| 2109 | NM_000249.3(MLH1): | 4292 | MLH1 | [′GCATCTTCTTACTCTTCATCAACCGHAAGTT |
| | c.790 + 2T > C | | | AAAAAGAACCACATGGGAAA′] |
| 2110 | NM_000251.2(MSH2): c.1319T > | 4436 | MSH2 | [′TTGTTGGCAGTTTTTGTGACTCCTCYTACTG |
| | C (p.Leu440Pro) | | | ATCTTCGTTCTGACTTCTCC′] |
| 2111 | NM_000251.2(MSH2): | 4436 | MSH2 | [′AACAGATGTTCCACATCATTACTGGYAAAA |
| | c.2005 + 2T > C | | | AACCTGGTTTTTGGGCTTTGT′] |
| 2112 | NM_000251.2(MSH2): c.2089T > | 4436 | MSH2 | [′GGCCCAAATTGGGTGTTTTGTGCCAYGTGAG |
| | C (p.Cys697Arg) | | | TCAGCAGAAGTGTCCATTGT′] |
| 2113 | NM_000251.2(MSH2): c.560T > C | 4436 | MSH2 | [′CCTGATAATGATCAGTTCTCCAATCBTGAGG |
| | (p.Leu187Pro) | | | CTCTCCTCATCCAGATTGGA′] |
| 2114 | NM_000251.2(MSH2): c.595T > C | 4436 | MSH2 | [′CCTCATCCAGATTGGACCAAAGGAAYGTGT |
| | (p.Cys199Arg) | | | TTTACCCGGAGGAGAGACTGC′] |
| 2115 | NM_000251.2(MSH2): c.929T > C | 4436 | MSH2 | [′TTGGATATTGCAGCAGTCAGAGCCCBTAACC |
| | (p.Leu310Pro) | | | TTTTTCAGGTAAAAAAAAAA′] |
| 2116 | NM_007294.3(BRCA1): | 672 | BRCA1 | [′AAAGTGTGACCACATATTTTGCAAGBAAGTT |
| | c.134 + 2T > C | | | TGAATGTGTTATGTGGCTCC′] |
| 2117 | NM_001194998.1(CEP152): | 22995 | CEP152 | [′TATGAGGAAGACATCCTGACTGTACYTGGG |
| | c.3149T > C (p.Leu1050Pro) | | | GTTCTTTTAAGTGATACCCAA′] |
| 2118 | NM_001271723.1(FBXO38): | 81545 | FBXO38 | [′AGGGGTGAATGTTCCTGAAATTCCTYGTATC |
| | c.616T > C (p.Cys206Arg) | | | CCAATGCTAAGGCACCTTTA′] |
| 2119 | NM_014495.3(ANGPTL3): | −1 | — | [′ACATCGAATAGATGGATCACAAAACYTCAA |
| | c.883T > C (p.Phe295Leu) | | | TGAAACGTGGGAGAACTACAA′] |
| 2120 | NM_003184.3(TAF2): c.1945T > | 6873 | TAF2 | [′CTCTCATAGCGGAGCTGATACTGCCRCATAA |
| | C (p.Trp649Arg) | | | AATCAGCTTGCTCAAATTCT′] |
| 2121 | NM_000016.5(ACADM): | 34 | ACADM | [′TTCTTCTAGTATCCAGTCCCCCTAAYTAGAA |
| | c.233T > C (p.Ile78Thr) | | | GAGCCTGGGAACTTGGTTTA′] |
| 2122 | NM_000019.3(ACAT1): | 38 | ACAT1 | [′TTCCTGTCACAGTTACAGTAAAAGGYAGAG |
| | c.730 + 2T > C | | | ATAATGTTCCAAAAAGGATGA′] |
| 2123 | NM_000143.3(FH): c.1255T > C | 2271 | FH | [′GAAGCATCCCCCAGCAGCCTGGCTGRGTGT |
| | (p.Ser419Pro) | | | AACACATTTTTAATCTTTGAG′] |
| 2124 | NM_000169.2(GLA): c.899T > C | −1 | — | [′GCTCCTTTATTCATGTCTAATGACCYCCGAC |
| | (p.Leu300Pro) | | | ACATCAGCCCTCAAGCCAAA′] |
| 2125 | NM_000202.6(IDS): c.587T > C | 3423 | IDS | [′GTGCTGGATGTTCCCGAGGGCACCTYGCCTG |
| | (p.Leu196Ser) | | | ACAAACAGAGCACTGAGCAA′] |
| 2126 | NM_000252.2(MTM1): c.688T > | 4534 | MTM1 | [′TCTGACTTAACCATAGGTGCTGTCAYGGATT |
| | C (p.Trp230Arg) | | | CATCCAGAAAATAAGACGGT′] |
| 2127 | NM_000277.1(PAH): c.638T > C | 5053 | PAH | [′GAGTACAATCACATTTTTCCACTTCYTGAAA |
| | (p.Leu213Pro) | | | AGTACTGTGGCTTCCATGAA′] |
| 2128 | NM_000404.2(GLB1): | 2720 | GLB1 | [′TTCTTCTCCGCTCCTCCGACCCAGGYAGGTT |
| | c.457 + 2T > C | | | GTTACAGATGTCTTGAGAAG′] |
| 2129 | NM_000528.3(MAN2B1): | 4125 | MAN2B1 | [′AGAGATGGCTCGCTGGAGCTCATGGYGAGT |
| | c.2436+ 2T > C | | | GGGTCAGAGCCCCATCCGAGC′] |
| 2130 | NM_000531.5(OTC): | 5009 | OTC | [′CTGGCTGATTACCTCACGCTCCAGGHTGGTT |
| | c.540 + 2T > C | | | TATTTATTTGTCTTACAAAA′] |
| 2131 | NM_001005741.2(GBA): | 2629 | GBA | [′TTCACCGCTCCATTGGTCTTGAGCCRAGTGG |
| | c.667T > C (p.Trp223Arg) | | | GTGATGTCCAGGGGCTGGCA′] |
| 2132 | NM_000402.4(G6PD): c.473T > C | 2539 | G6PD | [′CGCCTCAACAGCCACATGAATGCCCYCCAC |
| | (p.Leu158Pro) | | | CTGGGGTCACAGGCCAACCGC′] |
| 2133 | NM_000402.4(G6PD): c.188T > C | −1 | — | [′CACCGATGCACCCATGATGATGAATRTGTGT |
| | (p.Ile63Thr) | | | GTATCCGACTGATGGAAGGC′] |
| 2134 | NM_006920.4(SCN1A): | −1 | — | [′GGGTATCTCTCTTTGCTTCAAGTTGYAAGTG |
| | c.4251 + 2T > C | | | AACACTATTTTCTCTGAATA′] |
| 2135 | NM_002225.3(IVD): | 3712 | IVD | [′CAGAAAGAGAAGTATCTCCCGAAGGYGAGG |
| | c.465 + 2T > C | | | AAATGGAAATGTAATACACGC′] |
| 2136 | NM_003494.3(DYSF): | 8291 | DYSF | [′GAGGTCAGCTTTGCGGGGAAAATGGYAAGG |
| | c.1284 + 2T > C | | | AGCAAGGGAGCAGGAGGGTTC′] |
| 2137 | NM_004006.2(DMD): | 1756 | DMD | [′CCCTGGTGGAACAGATGGTGAATGGYAATT |
| | c.2380 + 2T > C | | | ACACGAGTTGATTTAGATAAT′] |
| 2138 | NM_012463.3(ATP6V0A2): | 23545 | ATP6V0A2 | [′ACCCGCATCCAGGATCTCTACACTGYGAGTA |
| | 5c.82 + 2T > C | | | AGCTGGAAGTGGATTGCCTC′] |
| 2139 | NM_014795.3(ZEB2): | 9839 | ZEB2 | [′AAGCCAATCCCAGGAGGAAAAACGGYAAG |
| | c.73 + 2T >C | | | AAGCAGCCCGAACCAAACTTTT′] |
| 2140 | NM_016725.2(FOLR1): | 2348 | FOLR1 | [′ACAAGGGCTGGAACTGGACTTCAGGYGAGG |
| | c.493 + 2T > C | | | GCTGGGGTGGGCAGGAATGGA′] |
| 2141 | NM_000056.3(BCKDHB): | 594 | BCKDHB | [′CCTGTTCTGTATTTAGCGGAAGAAGYCCCTA |
| | c.752T > C (p. Val251Ala) | | | TAGAACCATACAACATCCCA′] |
| 2142 | NM_175053.3(KRT74): c.821T > | 121391 | KRT74 | [′TACTGCATCATACAGACACTTGAGGRACTTG |
| | C (p.Phe274Ser) | | | ATTTCTTTGTCCAGTGAGTC′] |
| 2143 | NM_003764.3(STX11): c.173T > | 8676 | STX11 | [′GACATTCAGGATGAAAACCAGCTGCYGGTG |
| | C (p.Leu58Pro) | | | GCCGACGTGAAGCGGCTGGGA′] |
| 2144 | NM_001044.4(SLC6A3): | 6531 | SLC6A3 | [′CTCTGCAGACGTGGCGTGCTGCACCYCCACC |
| | c.671T > C (p.Leu224Pro) | | | AGAGCCATGGCATCGACGAC′] |
| 2145 | NM_015662.2(IFT172): | −1 | — | [′CTGATGAATTTCAGCACGTCCTGGCRCACTG |
| | c.5179T > C (p.Cys1727Arg) | | | GGCTGTGGGAGGTCTGTGAG′] |
| 2146 | NM_015662.2(IFT172): | 26160 | IFT172 | [′GCGCGTGGCATAGTAATGAGCGATCRGCAG |
| | c.4607T > C (p.Leu1536Pro) | | | CATCGTCTTGAACTCCTCATG′] |
| 2147 | NM_014754.2(PTDSS1): | 9791 | PTDSS1 | [′ACCGGGAAGATCAAGAGAGCTGTTCYGCAG |
| | c.794T > C (p.Leu265Pro) | | | TTCACTCCTGCTAGCTGGACC′] |
| 2148 | NM_014714.3(IFT140): | 9742 | IFT140 | [′GGACCCCAAGGAGTCCATCAAGCAGYGTGA |
| | c.4078T > C (p.Cys1360Arg) | | | GCTGCTCCTGGAGGAACCAGA′] |
| 2149 | NM_000531.5(OTC): | 5009 | OTC | [′GAAAACAGAAAGTGGACAATCATGGYAAGC |
| | c.1005 + 2T > C | | | AAGAAACAAGGAATGGAGGAT′] |
| 2150 | NM_000531.5(OTC): c.1018T > C | 5009 | OTC | [′GTTGTGTCATCAGGCTGTCATGGTGYCCCTG |
| | (p.Ser340Pro) | | | CTGACAGATTACTCACCTCA′] |
| 2151 | NM_000531.5(OTC): c.1022T > C | 5009 | OTC | [′TGTCATCAGGCTGTCATGGTGTCCCYGCTGA |
| | (p.Leu341Pro) | | | CAGATTACTCACCTCAGCTC′] |
| 2152 | NM_000531.5(OTC): c.1033T > C | 5009 | OTC | [′TGTCATGGTGTCCCTGCTGACAGATBACTCA |
| | (p.Tyr345His) | | | CCTCAGCTCCAGAAGCCTAA′] |
| 2153 | NM_000531.5(OTC): c.143T > C | 5009 | OTC | [′CGTGACCTTCTCACTCTAAAAAACTYTACCG |
| | (p.Phe48Ser) | | | GAGAAGAAATTAAATATATG′] |
| 2154 | NM_000531.5(OTC): c.158T > C | 5009 | OTC | [′CTAAAAAACTTTACCGGAGAAGAAABTAAA |
| | (p.Ile53Thr) | | | TATATGCTATGGCTATCAGCA′] |
| 2155 | NM_000531.5(OTC): c.167T > C | 5009 | OTC | [′TTTACCGGAGAAGAAATTAAATATAYGCTA |
| | (p.Met56Thr) | | | TGGCTATCAGCAGATCTGAAA′] |
| 2156 | NM_000531.5(OTC): c.188T > C | 5009 | OTC | [′TATATGCTATGGCTATCAGCAGATCYGAAAT |
| | (p.Leu63Pro) | | | TTAGGATAAAACAGAAAGGA′] |
| 2157 | NM_000531.5(OTC): c.227T > C | 5009 | OTC | [′GTCCTTGATTTATAGTATTTGCCTTYATTGCA |
| | (p.Leu76Ser) | | | AGGGAAGTCCTTAGGCATG′] |
| 2158 | NM_000531.5(OTC): c.284T > C | 5009 | OTC | [′GAGAAAAGAAGTACTCGAACAAGATYGTCT |
| | (p.Leu95Ser) | | | ACAGAAACAGGTAAGTCCACT′] |
| 2159 | NM_000531.5(OTC): c.2T > C | 5009 | OTC | [′CGTCCTTTACACAATTAAAAGAAGAYGCTGT |
| | (p.Met1Thr) | | | TTAATCTGAGGATCCTGTTA′] |
| 2160 | NM_000531.5(OTC): | 5009 | OTC | [′TGAAAGTCTCACGGACACGGCCCGGYTTGT |
| | c.386 + 2T > C | | | AAATATTTTCTTCTCTCCAAA′] |
| 2161 | NM_000531.5(OTC): c.392T > C | 5009 | OTC | [′TTTTTCTTGGTTTGCCACAGTGTATYGTCTAG |
| | (p.Leul31Ser) | | | CATGGCAGATGCAGTATTG′] |
| 2162 | NM_000531.5(OTC): c.394T > C | 5009 | OTC | [′TTTCTTGGTTTGCCACAGTGTATTGYCTAGC |
| | (p.Ser132Pro) | | | ATGGCAGATGCAGTATTGGC′] |
| 2163 | NM_000531.5(OTC): c.416T > C | 5009 | OTC | [′TTGTCTAGCATGGCAGATGCAGTATYGGCTC |
| | (p.Leu139Ser) | | | GAGTGTATAAACAATCAGAT′] |
| 2164 | NM_000531.5(OTC): c.443T > C | 5009 | OTC | [′GCTCGAGTGTATAAACAATCAGATTBGGAC |
| | (p.Leu148Ser) | | | ACCCTGGCTAAAGAAGCATCC′] |
| 2165 | NM_000531.5(OTC): c.476T > C | 5009 | OTC | [′CTGGCTAAAGAAGCATCCATCCCAAYTATC |
| | (p.Ile159Thr) | | | AATGGGCTGTCAGATTTGTAC′] |
| 2166 | NM_000531.5(OTC): c.479T > C | 5009 | OTC | [′GCTAAAGAAGCATCCATCCCAATTANCAAT |
| | (p.Ile160Thr) | | | GGGCTGTCAGATTTGTACCAT′] |
| 2167 | NM_000531.5(OTC): c.490T > C | 5009 | OTC | [′ATCCATCCCAATTATCAATGGGCTGYCAGAT |
| | (p.Ser164Pro) | | | TTGTACCATCCTATCCAGAT′] |
| 2168 | NM_000531.5(OTC): c.526T > C | 5009 | OTC | [′CCATCCTATCCAGATCCTGGCTGATYACCTC |
| | (p.Tyr176His) | | | ACGCTCCAGGTTGGTTTATT′] |
| 2169 | NM_000531.5(OTC): c.536T > C | 5009 | OTC | [′CAGATCCTGGCTGATTACCTCACGCYCCAGG |
| | (p.Leul79Pro) | | | TTGGTTTATTTATTTGTCTT′] |
| 2170 | NM_000531.5(OTC): c.577T > C | 5009 | OTC | [′CTCTCTGAAAGGTCTTACCCTCAGCBGGATC |
| | (p.Trp193Arg) | | | GGGGATGGGAACAATATCCT′] |
| 2171 | NM_000531.5(OTC): c.602T > C | 5009 | OTC | [′TGGATCGGGGATGGGAACAATATCCYGCAC |
| | (p.Leu201Pro) | | | TCCATCATGATGAGCGCAGCG′] |
| 2172 | NM_000531.5(OTC): | 5009 | OTC | [′CACCTTCAGGCAGCTACTCCAAAGGYAGGG |
| | c.663 + 2T > C | | | AAACTTTTTGCCTTGAAACTA′] |
| 2173 | NM_000531.5(OTC): c.779T > C | 5009 | OTC | [′GAAGCAGCGCATGGAGGCAATGTATYAATT |
| | (p.Leu260Ser) | | | ACAGACACTTGGATAAGCATG′] |
| 2174 | NM_000531.5(OTC): c.793T > C | 5009 | OTC | [′AGGCAATGTATTAATTACAGACACTYGGAT |
| | (p.Trp265Arg) | | | AAGCATGGGACAAGAAGAGGA′] |
| 2175 | NM_000531.5(OTC): c.803T > C | 5009 | OTC | [′TTAATTACAGACACTTGGATAAGCAYGGGA |
| | (p.Met268Thr) | | | CAAGAAGAGGAGAAGAAAAAG′] |
| 2176 | NM_000531.5(OTC): c.907T > C | 5009 | OTC | [′TGCCTCTGACTGGACATTTTTACACBGCTTG |
| | (p.Cys303Arg) | | | CCCAGAAAGCCAGAAGAAGT′] |
| 2177 | NM_000531.5(OTC): c.947T > C | 5009 | OTC | [′CCAGAAGAAGTGGATGATGAAGTCTYTTAT |
| | (p.Phe316Ser) | | | TCTCCTCGATCACTAGTGTTC′] |
| 2178 | NM_000322.4(PRPH2): c.637T > | 5961 | PRPH2 | [′CCTGGTGGACGGCGTCCCTTTCAGCYGCTGC |
| | C (p.Cys213Arg) | | | AATCCTAGCTCGCCACGGCC′] |
| 2179 | NM_000322.4(PRPH2): c.736T > | 5961 | PRPH2 | [′CCACCAGACGGAGGAGCTCAACCTGYGGGT |
| | C (p.Trp246Arg) | | | GCGTGGCTGCAGGGCTGCCCT′] |
| | NM_017415.2(KLHL3): | 26249 | KLHL3 | [] |
| | c.1160T > C (p.Leu387Pro) | | | |
| | NM_017415.2(KLHL3): | 26249 | KLHL3 | [] |
| | c.1280T > C (p.Met427Thr) | | | |
| 2180 | NM_003859.1(DPM1): c.742T > C | −1 | — | [′AGAGTCAATAATCCTTTCAAGAAAGRTACTA |
| | (p.Ser248Pro) | | | TTTCATTTCCTCCCAACTTG′] |
| 2181 | NM_004826.3(ECEL1): | 9427 | ECEL1 | [′GGGTTCATGGGTGAGTCCTTGGGACRGTGG |
| | c.2278T > C (p.Cys760Arg) | | | AAAGCCCGGCCAAACTCCTCA′] |
| 2182 | NM_014908.3(DOLK): c.2T > C | 22845 | DOLK | [′GGCCGGAGATGGGCACTCTCGGGTCRTATCT |
| | (p.Met1Thr) | | | CTAGACCTGGGGCTTCACGG′] |
| 2183 | NM_000404.2(GLB1): c.922T > C | 2720 | GLB1 | [′TCTTTTGTTTCCTTGTAGGTACATGYTTATAG |
| | (p.Phe308Leu) | | | GTGGGACCAATTTTGCCTA′] |
| 2184 | NM_012434.4(SLC17A5): | 26503 | SLC17A5 | [′TTAGGAGTTGGACCACTCATTGTACYCAGAG |
| | c.500T > C (p.Leu167Pro) | | | CACTAGAAGGACTAGGAGAG′] |
| 2185 | NM_000211.4(ITGB2): | 3689 | ITGB2 | [′CCCCTCACCCTGTGGCAAGTACATGYGAGTG |
| | c.1877 + 2T > C | | | CAGGCGGAGCAGGCAGGGCG′] |
| 2186 | NM_006702.4(PNPLA6): | 10908 | PNPLA6 | [′ACCTCCATGTTCACTGGGTCTGCCTYTAACC |
| | c.3053T > C (p.Phe1018Ser) | | | GCAGCATCCATCGGGTCTTC′] |
| 2187 | NM_176787.4(PIGN): c.808T > C | 23556 | PIGN | [′TCTGAAGGATGACCAGCCCCATGGGRACCT |
| | (p.Ser270Pro) | | | ACAAATAAGATATAAAGAATA′] |
| 2188 | NM_001165899.1(PDE4D): | 5144 | PDE4D | [′CCATGTCTCCCAGAGGGGATGAACARTATA |
| | c.1850T > C (p.Ile617Thr) | | | GTCTATGAAGCCCACCTAGTT′] |
| 2189 | NM_005017.3(PCYT1A): | 5130 | PCYT1A | [′CCTTCTGTCCTCTGTGTTGGAGCAARCATGC |
| | c.571T > C (p.Phe191Leu) | | | CTAACTCAGAAACACATACA′] |
| 2190 | NM_000090.3(COL3A1): | 1281 | COL3A1 | [′CAGGACTTCCTGGGGCTGCAGTGAGHATAG |
| | c.951 + 6T > C | | | CTGCTAACATCACACAATTAC′] |
| | (p.Gly300_Ala317del) | | | |
| 2191 | NM_000090.3(COL3A1): | 1281 | COL3A1 | [′GCACCTGGAGCTCCAGGAGGCAAGGYAGTA |
| | c.2022 + 2T > C | | | TTTCAATTTATTCTCTACCTT′] |
| | (p.Gly660_Lys674del) | | | |
| 2192 | NM_000090.3(COL3A1): | 1281 | COL3A1 | [′CGGCCAGGACTTCCTGGGGCTGCAGHGAGT |
| | c.951 + 2T > C | | | ATAGCTGCTAACATCACACAA′] |
| | (p.Gly300_Ala317del) | | | |
| 2193 | NM_000090.3(COL3A1): | 1281 | COL3A1 | [′CGAGATGGATCTCCTGGTGGCAAGGBATAA |
| | c.3093 + 2T > C | | | TAAACACATGTGCAATTGATT′] |
| | (p.Gly1014_Lys1031del) | | | |
| 2194 | NM_000090.3(COL3A1): | 1281 | COL3A1 | [′CCAGCTGGCCAGCCTGGAGATAAGGYAACC |
| | c.2337 + 2T > C | | | CTTAATACTACCTGGATATAA′] |
| | (p.Gly762_Lys779del) | | | |
| 2195 | NM_000090.3(COL3A1): | 1281 | COL3A1 | [′TTCCCCGGTCCTAAAGGAAATGATGBGAGTT |
| | c.1761 + 2T > C | | | CCTTCATTAATTTCTTCAAT′] |
| | (p.Gly555_Asp587del) | | | |
| 2196 | NM_000090.3(COL3A1): | 1281 | COL3A1 | [′CTGGTGAACCTGGAAGAGATGTGAGYAGCA |
| | c.3039 + 6T > C | | | GTTTTTATTCAACCAGCCAGG′] |
| | (p.Asp1013_ | | | |
| | Gly1014insVSSSFYSTSQ) | | | |
| 2197 | NM_000090.3(COL3A1): c.4399 | 1281 | COL3A1 | [′GGACGTTGGCCCTGTTTGCTTTTTAYAAACC |
| | T > C (p.Ter1467Gln) | | | AAACTCTATCTGAAATCCCA′] |
| 2198 | NM_000090.3(COL3A1): | 1281 | COL3A1 | [′CCCCCTGGAGGTTCTGGACCTGCTGYAAGTT |
| | c.2553 + 2T > C | | | CCTTCCTCTTTCTCTGTCTA′] |
| | (p.Gly816_Ala851del) | | | |
| 2199 | NM_001278503.1(STT3A): | 3703 | STT3A | [′TATACTCCAACTGGGGAGTTCCGTGYGGACC |
| | c.1877T > C (p.Val626Ala) | | | GTGAAGGTTCTCCAGTGCTG′] |
| 2200 | NM_000277.1(PAH): c.691T > C | 5053 | PAH | [′TAACATTCCCCAGCTGGAAGACGTTYCTCAG |
| | (p.Ser231Pro) | | | TTCCTGCAGAGTAAGTCCAC′] |
| 2201 | NM_007294.3(BRCA1): | 672 | BRCA1 | [′GGACAGAGGACAATGGCTTCCATGGBAAGG |
| | c.5467 + 2T > C | | | TGCCTGCATGTACCTGTGCTA′] |
| 2202 | NM_020347.3(LZTFL1): | 54585 | LZTFL1 | [′ATACCACTTCTCAGCTTGTGCAAACRGCTGT |
| | c.260T > C (p.Leu87Pro) | | | CGCAGAAGTAACACATTGGT′] |
| 2203 | NM_016381.5(TREX1): c.530T > | 11277 | TREX1 | [′CGCCAGCCACAGCCCTGGTGCCTGGYGGCA |
| | C (p.Val177Ala) | | | CACAATGGTGACCGCTACGAC′] |
| 2204 | NM_015474.3(SAMHD1): | 25939 | SAMHD1 | [′TTTGTGTTGATAAGCTCTACGGTGTRAAGAG |
| | c.1106T > C (p.Leu369Ser) | | | TTGCGAGTGTGGAACATGTC′] |
| 2205 | NM_005654.5(NR2F1): c.755T > | 7025 | NR2F1 | [′CAGATCACCGACCAGGTGTCCCTGCYACGC |
| | C (p.Leu252Pro) | | | CTCACCTGGAGCGAGCTGTTC′] |
| 2206 | NM_003638.2(ITGA8): | 8516 | ITGA8 | [′TGCCTATATATATTTAAAGATACTCRCTACT |
| | c.2982 + 2T > C | | | ATGCTTCCTTCTGGGAGTTT′] |
| 2207 | NM_000321.2(RB1): | 5925 | RB1 | [′AGGATCAGATGAAGCAGATGGAAGGYAGG |
| | c.2663 + 2T > C | | | AACCAGTTTTGAATGTTTTCCA′] |
| 2208 | NM_000321.2(RB1): c. 1472T > C | 5925 | RB1 | [′CATATGTCTTTATTGGCGTGCGCTCYTGAGG |
| | (p.Leu491Pro) | | | TTGTAATGGCCACATATAGC′] |
| 2209 | NM_015884.3(MBTPS2): | 51360 | MBTPS2 | [′GCTATTGTTAATGCAGTACCCTGCTYTGCTT |
| | c.1391T > C (p.Phe464Ser) | | | TGGATGGACAATGGATTCTA′] |
| 2210 | NM_001739.1(CA5A): c.697T > C | 763 | CA5A | [′TCGGTCAGCGGCGGGGTGGTGAGCGRGCCC |
| | (p.Ser233Pro) | | | GCGTAGGTCCAGTAATCCCAG′] |
| 2211 | NM_005051.2(QARS): c.169T > C | 5859 | QARS | [′TCCCTGAGTCGGGAGGCCAAGCCATRTAAC |
| | (p.Tyr57His) | | | AGGATCCCGGTAGCTTTGTCA′] |
| 2212 | NM_005356.4(LCK): c. 1022T > C | 3932 | LCK | [′GGCATCAAGTTGACCATCAACAAACYCCTG |
| | (p.Leu341Pro) | | | GACATGGCAGCCCAAGTAAGG′] |
| 2213 | NM_021803.3(IL21): c. 146T > C | 59067 | IL21 | [′TACCAAGTCATTCACATAATTTTTCRGCTGA |
| | (p.Leu49Pro) | | | TCAACAATATCTATAAGTTG′] |
| 2214 | NM_005861.3(STUB1): c.719T > | −1 | — | [′TGTGGCAAGATCAGCTTTGAGCTGAYGCGG |
| | C (p.Met240Thr) | | | GAGCCGTGCATCACGCCCAGT′] |
| 2215 | NM_032575.2(GLIS2): c.523T > C | 84662 | GLIS2 | [′TCAGAACACTTCCCATCCTCCGCAGYGTAAC |
| | (p.Cys175Arg) | | | CAGCTCTTTGAGCTCCTGCA′] |
| 2216 | NM_001101.3(ACTB): c.224T > C | 60 | ACTB | [′CTGAAGTACCCCATCGAGCACGGCAYCGTC |
| | (p.Ile75Thr) | | | ACCAACTGGGACGACATGGAG′] |
| 2217 | NM_001101.3(ACTB): c.356T > C | 60 | ACTB | [′AACCCCAAGGCCAACCGCGAGAAGAYGACC |
| | (p.Met119Thr) | | | CAGGTGAGTGGCCCGCTACCT′] |
| 2218 | NM_003401.3(XRCC4): c.127T > | 7518 | XRCC4 | [′TACACTTACTGATGGTCATTCAGCAYGGACT |
| | C (p.Trp43Arg) | | | GGGACAGGTAATACTAAAAA′] |
| 2219 | NM_000051.3(ATM): | 472 | ATM | [′CTGGAGAGAGCCAAAGTACCATAGGYAAAT |
| | c.2638 + 2T > C | | | ACATATTTACTACTTGGGATT′] |
| 2220 | NM_000535.5(PMS2): c.2T > C | 5395 | PMS2 | [′CGAGGCGGATCGGGTGTTGCATCCAHGGAG |
| | (p.Met1Thr) | | | CGAGCTGAGAGCTCGAGGTGA′] |
| 2221 | NM_015713.4(RRM2B): c.190T > | 50484 | RRM2B | [′ATGCCACGTACCTCTTCTGCTGTCCRGAAGG |
| | C (p.Trp64Arg) | | | AAGCCTGTGCCTGTTTATAC′] |
| 2222 | NM_015713.4(RRM2B): c.368T > | 50484 | RRM2B | [′CTCGATGAGAATTTGAAAGCCATAGRAACA |
| | C (p.Phe123Ser) | | | GCGAGCCTCTGGAACCTGCAC′] |
| 2223 | NM_003122.4(SPINK1): | 6690 | SPINK1 | [′TTAAAAGAAACTCAAGTTTGTACTCRCCGAT |
| | c.194 + 2T > C | | | TTTCAAAACATAACACGCAT′] |
| 2224 | NM_003122.4(SPINK1): c.160T > | 6690 | SPINK1 | [′AAACATAACACGCATTCATTGGGATRAGTAT |
| | C (p.Tyr54His) | | | TTCCATCAGTCCCACAGACA′] |
| | NM_000219.5(KCNE1): c.158T > | 3753 | KCNE1 | [] |
| | C (p.Phe53Ser) | | | |
| 2225 | NM_000219.5(KCNE1): c.176T > | 3753 | KCNE1 | [′GCGGATGTAGCTCAGCATGATGCCCRGGGT |
| | C (p.Leu59Pro) | | | GAAGAAGCCGAAGAATCCCAG′] |
| | NM_000219.5(KCNE1): c.259T > | 3753 | KCNE1 | [] |
| | C (p.Trp87Arg) | | | |
| 2226 | NM_017890.4(VPS13B): | 157680 | VPS13B | [′CGTTTGTAAAGCACATCTCCAAAGGYAGCG |
| | c.11119 + 2T > C | | | GGTTCCGTTCCTTGTAATAAT′] |
| 2227 | NM_005026.3(PIK3CD): | 5293 | PIK3CD | [′CACCTGCCCTGTCCTTCTGCAGGACYGCCCC |
| | c.1246T > C (p.Cys416Arg) | | | ATTGCCTGGGCCAACCTCAT′] |
| 2228 | NM_000271.4(NPC1): c.2054T > | 4864 | NPC1 | [′TACATTGGGTTGCCCTTGACCCTCAYTGTGA |
| | C (p.Ile685Thr) | | | TTGAAGTCATCCCGTTCCTG′] |
| 2229 | NM_002633.2(PGM1): c.1547T > | 5236 | PGM1 | [′GGGAGTGCCGGGGCCACCATTCGGCYGTAC |
| | C (p.Leu516Pro) | | | ATCGATAGCTATGAGAAGGAC′] |
| 2230 | NM_015599.2(PGM3): c.248T > C | 5238 | PGM3 | [′TTGGTTGATCCTTTGGGTGAAATGTYGGCAC |
| | (p.Leu83Ser) | | | CATCCTGGGAGGAACATGCC′] |
| 2231 | NM_002136.2(HNRNPA1): | 3178 | HNRNPA1 | [′GAATTACAACAATCAGTCTTCAAATBTTGGA |
| | c.817T > C (p.Phe273Leu) | | | CCCATGAAGGGAGGAAATTT′] |
| 2232 | NM_002136.2(HNRNPA1): | 3178 | HNRNPA1 | [′TTTTGGACCCATGAAGGGAGGAAATYTTGG |
| | c.841T > C (p.Phe281Leu) | | | AGGCAGAAGCTCTGGCCCCTA′] |
| 2233 | NM_001159287.1(TPI1): | 7167 | TPI1 | [′GGTGGTGCTTCCCTCAAGCCCGAATYCGTGG |
| | c.833T > C (p.Phe278Ser) | | | ACATCATCAATGCCAAACAA′] |
| 2234 | NM_130838.1(UBE3A): c.2T > C | 7337 | UBE3A | [′TCTGACGACATTGAAGCTAGCCGAAYGTAA |
| | (p.Met1Thr) | | | GTGTAACTTGGTTGAGACTGT′] |
| 2235 | NM_130838.1(UBE3A): c.710T > | 7337 | UBE3A | [′ATTGAAACTGCCTTTCTCAATGCACHTGTAT |
| | C (p.Leu237Pro) | | | ATTTGTCACCTAACGTGGAA′] |
| 2236 | NM_000414.3(HSD17B4): | 3295 | HSD17B4 | [′AGTGGAGACTGGAATCCCTTACACAYTGAT |
| | c.1547T > C (p.Ile516Thr) | | | CCTAACTTTGCTAGTCTAGCA′] |
| 2237 | NM_022068.3(PIEZO2): | 63895 | PIEZO2 | [′GTCCATTTGATCATTGTCTCTGGTGRGCGAT |
| | c.8215T > C (p.Ser2739Pro) | | | ATAGGAATATTAATTTGGCA′] |
| 2238 | NM_012079.5(DGAT1): | 8694 | DGAT1 | [′GCACCTCAGGCCCACAGAGGTCCTCRCCGC |
| | c.751 + 2T > C | | | GGTAGGTCAGATTGTCCGGGT′] |
| 2239 | NM_003108.3(SOX11): c.178T > | 6664 | SOX11 | [′GCCGATGAACGCGTTCATGGTATGGYCCAA |
| | C (p.Ser60Pro) | | | GATCGAACGCAGGAAGATCAT′] |
| 2240 | NM_021072.3(HCN1): c.814T > C | 348980 | HCN1 | [′TGATGTATGTATCTAATTAACCTTGRAAGTC |
| | (p.Ser272Pro) | | | GTAATAAACGCAAGAGACTG′] |
| 2241 | NM_001079867.1(PEX2): | 5828 | PEX2 | [′CACCAGTGGCAAAGAATGCGCTCTAYGTGG |
| | c.739T > C (p.Cys247Arg) | | | AGAGTGGCCCACCATGCCTCA′] |
| 2242 | NM_022552.4(DNMT3A): | 1788 | DNMT3A | [′CTGAATGCCCAAGTCCTTCAGCACCRGGAGC |
| | c.1943T > C (p.Leu648Pro) | | | CCTGCACCAGCCAGCAGACA′] |
| 2243 | NM_022552.4(DNMT3A): | 1788 | DNMT3A | [′CGCAAAATACTCCTTCAGCGGAGCGRAGAG |
| | c.2705T > C (p.Phe902Ser) | | | GTGGCGGATGACTGGCACGCT′] |
| 2244 | NM_018400.3(SCN3B): c.482T > | 55800 | SCN3B | [′GAGGAAGACCAGAAGGATGTACATCRTGAT |
| | C (p.Met161Thr) | | | TTCTGAGACCACAGAGGTGAA′] |
| 2245 | NM_000406.2(GNRHR): | 2798 | GNRHR | [′TTCTCCATGTATGCCCCAGCCTTCAYGATGG |
| | c.392T > C (p.Met131Thr) | | | TGGTGATCAGCCTGGACCGC′] |
| 2246 | NM_001030001.2(RPS29): | 6235 | RPS29 | [′GCAGACGCCTACCTTAATGAAACCGRTATCC |
| | c.149T > C (p.Ile50Thr) | | | TTCGCGTACTGACGGAAACA′] |
| 2247 | NM_177550.4(SLC13A5): | 284111 | SLC13A5 | [′CAGGGTACAGGGCAGCATGATGTACRGCGG |
| | c.1463T > C (p.Leu488Pro) | | | ATTGAGGCCGATGGAGCGAGA′] |
| 2248 | NM_004464.3(FGF5): c.520T > C | 2250 | FGF5 | [′TTTTCAAGAAAATAGCTATAATACCBATGCC |
| | (p.Tyr174His) | | | TCAGCAATACATAGAACTGA′] |
| 2249 | NM_000051.3(ATM): | 472 | ATM | [′AGAGGACCCTTTTCACTCTTGGAGGYAATAA |
| | c.4776 + 2T > C | | | AAATTTCATCATCTACTATT′] |
| 2250 | NM_002880.3(RAF1): c.1808T > | 5894 | RAF1 | [′GTGTTGGAGCAGCTCAATGGAAGACRGGAT |
| | C (p.Leu603Pro) | | | CTGAAACAAAGCCCAAGAATG′] |
| 2251 | NM_000251.2(MSH2): | 4436 | MSH2 | [′GTCAGAGCCCTTAACCTTTTTCAGGBAAAAA |
| | c.942 + 2T > C | | | AAAAAAAAAAAAAAAAAAAA′] |
| 2252 | NM_001077620.2(PRCD): c.2T > | −1 | — | [′GGGAGGGGATGGGGCAGCTGCGCCAYGTGC |
| | C (p.Met1Thr) | | | ACCACCCTTTTCCTGCTCAGC′] |
| 2253 | NM_001142800.1(EYS): | 34600 | EYS | [′AGCCACAAAGTTTTTATGTGGATCARTATCC |
| | c.9209T > C (p.Ile3070Thr) | 7 | | TCGGAAAGAATTAGACTGTT′] |
| 2254 | NM_206933.2(USH2A): | 7399 | USH2A | [′TCCTAAATTGACAGGTGAAGTATGCYGTCCA |
| | c.9751T > C (p.Cys3251Arg) | | | GATGAACAGCACAATCGGGT′] |
| 2255 | NM_000257.3(MYH7): c.4442T > | −1 | — | [′GAGGCTCGCTCCCTCAGCACAGAGCYCTTCA |
| | C (p.Leu1481Pro) | | | AACTCAAGAACGCCTATGAG′] |
| 2256 | NM_000257.3(MYH7): c.4835T > | −1 | — | [′GAGACACGCAGCCGCAACGAGGCCCYGAGG |
| | C (p.Leu1612Pro) | | | GTGAAGAAGAAGATGGAAGGA′] |
| 2257 | NM_000257.3(MYH7): c.4937T > | −1 | — | [′GAGGCCCAGAAGCAAGTCAAGAGCCYCCAG |
| | C (p.Leu1646Pro) | | | AGCTTGTTGAAGGTACTCACC′] |
| 2258 | NM_001199138.1(NLRC4): c.10 | 58484 | NLRC4 | [′AATCTCATGAAGACCCCTCTCTTTGYGGTCA |
| | 22T > C (p.Val341Ala) | | | TCACTTGTGCAATCCAGATG′] |
| 2259 | NM_000076.2(CDKNIC): | 1028 | CDKNIC | [′GCGCAAGAGGCTGCGGTGAGCCAAGYGAGT |
| | c.*5 + 2T > C | | | ACAGCGCACCTGGGGGGGCGC′] |
| 2260 | NM_003159.2(CDKL5): c.659T > | 6792 | CDKL5 | [′CCTGGAGAAAGTGAAATTGACCAACYTTTT |
| | C (p.Leu220Pro) | | | ACTATTCAGAAGGTGCTAGGA′] |
| 2261 | NM_005027.3(PIK3R2): | 5296 | PIK3R2 | [′CTCACCTTCTGCTCCGTTGTGGACCYCATCA |
| | c.1202T > C (p.Leu401Pro) | | | ATCACTACCGCCACGAGTCT′] |
| 2262 | NM_173596.2(SLC39A5): | 283375 | SLC39A5 | [′TTCCTGCTCTTTGTGCTGGAGAACAYGCTGG |
| | c.911T > C (p.Met304Thr) | | | GGCTTTTGCGGCACCGAGGG′] |
| 2263 | NM_153334.6(SCARF2): | 91179 | SCARF2 | [′CCTTGCTGCCTCCAGCCAGCGCAGCRCGTGG |
| | c.190T > C (p.Cys64Arg) | | | GCACCTGGGAGCTGCGAGCA′] |
| 2264 | NM_001288767.1(ARMC5): | 79798 | ARMC5 | [′GGGGCACTTGTGACCGGCCCGGCGCYGTAC |
| | c.1928T > C (p.Leu643Pro) | | | GGCCTGCTGACCTATGTGACC′] |
| 2265 | NM_001288767.1(ARMC5): | 79798 | ARMC5 | [′CGTGAGGCCATCAACCGGGCCCGACYGCGG |
| | c.1379T > C (p.Leu460Pro) | | | GATGCTGGTGGCTTGGATCTA′] |
| 2266 | NC_012920.1: m.9478T > C | 4514 | MT-CO3 | [′ATAATCCTATTTATTACCTCAGAAGYTTTTTT |
| | | | | CTTCGCAGGATTTTTCTGA′] |
| 2267 | NC_012920.1: m.11984T > C | 4538 | MT-ND4 | [′ACTAGTCACAGCCCTATACTCCCTCYACATA |
| | | | | TTTACCACAACACAATGGGG′] |
| 2268 | NM_014845.5(FIG4): c.50T > C | 9896 | FIG4 | [′ATCAGCTCGGTCCAGAAGCTGGTTCYGTATG |
| | (p.Leul7Pro) | | | AGACTAGAGCTGTGAGTACC′] |
| 2269 | NM_003159.2(CDKL5): | 6792 | CDKL5 | [′TCAAGAAATTCAAGGACAGTGAAGGYAGAT |
| | c.145 + 2T > C | | | ATATATATATATATATATATA′] |
| 2270 | NM_002775.4(HTRA1): | 5654 | HTRA1 | [′CCATCTGATAAGATTAAAAAGTTCCYCACG |
| | c.1091T > C (p.Leu364Pro) | | | GAGTCCCATGACCGACAGGCC′] |
| 2271 | NM_021870.2(FGG): c.1210T > C | 2266 | FGG | [′ATCTTCATAGTGGTTTTCTTCATGGRATACC |
| | (p.Ser404Pro) | | | ACCGGGTTTTCCAAGTGGCC′] |
| 2272 | NM_017617.3(NOTCH1): | 4851 | NOTCH1 | [′TCGAAGGAGCCCAGCGTGTTGATGCRCTTGC |
| | c.1285T > C (p.Cys429Arg) | | | CCGCATGCTCGCAGGGGTTG′] |
| 2273 | NM_024915.3(GRHL2): | 79977 | GRHL2 | [′GATGATTCAGATTGACACATACAGTYATAA |
| | c.1192T > C (p.Tyr398His) | | | CAATCGTAGCAATAAACCCAT′] |
| 2274 | NM_002049.3(GATA1): c.2T > C | 2623 | GATA1 | [′CGCAGGTTAATCCCCAGAGGCTCCAYGGAG |
| | (p.Met1Thr) | | | TTCCCTGGCCTGGGGTCCCTG′] |
| 2275 | NM_002049.3(GATA1): | 2623 | GATA1 | [′TACTGTGGTGGCTCCGCTCAGCTCAYGAGGG |
| | c.1240T> C (p.Ter414Arg) | | | CACAGAGCATGGCCTCCAGA′] |
| 2276 | NM_005859.4(PURA): c.299T > C | 5813 | PURA | [′GGCGCGGGCGGCAACAAGAGCCGCCYTACT |
| | (p.Leu100Pro) | | | CTCTCCATGTCAGTGGCCGTG′] |
| 2277 | NM_032374.4(APOPT1): | 84334 | APOPT1 | [′TTCTGGGCAAACCAGAATTTGACTTYTAGTA |
| | c.353T > C (p.Phe118Ser) | | | AGGTAAGTTTAAGTTTTAGA′] |
| 2278 | NM_032551.4(KISS1R): c.937T > | 84634 | KISS1R | [′TAAGACCTGGGCTCACTGCATGTCCYACAGC |
| | C (p.Tyr313His) | | | AACTCCGCGCTGAACCCGCT′] |
| 2279 | NM_017696.2(MCM9): | 254394 | MCM9 | [′AAAGCTTGATACGATTAGCAGAAGGYCTAT |
| | c.1732 + 2T > C | | | TTCATTCAGCGAATGATGCTT′] |
| 2280 | NM_003159.2(CDKL5): c.602T > | 6792 | CDKL5 | [′GACATGTGGTCGGTGGGCTGTATTCYTGGGG |
| | C (p.Leu201Pro) | | | AGCTTAGCGATGGACAGCCT′] |
| 2281 | NM_005740.2(DNAL4): | 10126 | DNAL4 | [′GAGAAATTCTCCAACAACAACGAGGYATTG |
| | c.153 + 2T > C | | | CCAGCAGTGCAGGCGGCCCCT′] |
| 2282 | NM_201631.3(TGM5): c.763T > C | 9333 | TGM5 | [′TTCAGGATGGCCACGCTGCCCGTCCRCTCCG |
| | (p.Trp255Arg) | | | CAGGGTTGGCGCCGTCTGTG′] |
| 2283 | NM_201631.3(TGM5): c.122T > C | 9333 | TGM5 | [′GCTCCGGTTCCTGAAGTACAGGGTGRGGTTG |
| | (p.Leu41Pro) | | | AAGGCCTGGCCCCGGCGAAC′] |
| 2284 | NM_001287223.1(SCN11A): | 11280 | SCN11A | [′TACCTTCATTCCTTCAAACTGGGACRGCGCA |
| | c.3473T > C (p.Leu1158Pro) | | | CGAAGAGGCCTCAGTGCTCG′] |
| 2285 | NM_001287223.1(SCN11A): | 11280 | SCN11A | [′GGGCTCTACTCAGTCTTCTTCTTCAYTGTGG |
| | c.1142T > C (p.Ile381Thr) | | | TCATTTTCCTGGGCTCCTTC′] |
| 2286 | NM_001302946.1(TRNT1): | 51095 | TRNT1 | [′CATGATCCTGAGACTTTGGAAGCAAYTGCA |
| | c.668T > C (p.Ile223Thr) | | | GAAAATGCAAAAGGCTTGGCT′] |
| 2287 | NM_001302946.1(TRNT1): | 51095 | TRNT1 | [′TAATGAATAGGTTTTGATGGCACTTYATTTG |
| | c.497T > C (p.Leu166Ser) | | | ACTACTTTAATGGTTATGAA′] |
| 2288 | NM_018136.4(ASPM): | 259266 | ASPM | [′ATAGACACCTATGGAAAGATGTGGGYAAGA |
| | c.2419 + 2T > C | | | AGACTGCAGAAATCTTGACAT′] |
| 2289 | NM_022455.4(NSD1): c.5885T > | 64324 | NSD1 | [′TGGGGTCTACGGACAAAAACAGATAYTAAA |
| | C (p.Ile1962Thr) | | | AAGGTTAGAAAAAGCTAAATT′] |
| 2290 | NM_022455.4(NSD1): c.5989T > | 64324 | NSD1 | [′TCAAGAACATGATATCACTAATTTCYATATG |
| | C (p.Tyr1997His) | | | CTCACCCTAGACAAAGTAAG′] |
| 2291 | NM_133433.3(NIPBL): | 25836 | NIPBL | [′AAAAAATATGCTGGATTCATTCATGYATGTA |
| | c.7062 + 2T > C | | | TTTTAACATTTTATAACCTA′] |
| 2292 | NM_000525.3(KCNJ11): | 3767 | KCNJ11 | [′GGAGGACGGACGTTACTCTGTGGACYACTC |
| | c.988T > C (p.Tyr330His) | | | CAAGTTTGGCAACACCGTCAA′] |
| 2293 | NM_004004.5(GJB2): c.107T > C | 2706 | GJB2 | [′CTCTTCATTTTTCGCATTATGATCCYCGTTGT |
| | (p.Leu36Pro) | | | GGCTGCAAAGGAGGTGTGG′] |
| 2294 | NM_130838.1(UBE3A): | 7337 | UBE3A | [′CTTTAATGTGCTTTTACTTCCGGAAYACTCA |
| | c.2485T > C (p.Tyr829His) | | | AGCAAAGAAAAACTTAAAGA′] |
| 2295 | NM_005682.6(ADGRG1): | 9289 | ADGRG1 | [′GCCATCTTCCTGCACTTCTCCCTGCYCACCT |
| | c.1460T > C (p.Leu487Pro) | | | GCCTTTCCTGGATGGGCCTC′] |
| 2296 | NM_000430.3(PAFAH1B1): | 5048 | PAFAH1B1 | [′CCGAGAGCATGAGCATGTGGTAGAAYGCAT |
| | c.841T > C (p.Cys281Arg) | | | TTCCTGGGCTCCAGAAAGCTC′] |
| 2297 | NM_001005360.2(DNM2): | 1785 | DNM2 | [′GTGGACAGCTGGAAGGCCTCGTTCCYCCGA |
| | c.1862T > C (p.Leu621Pro) | | | GCTGGCGTCTACCCCGAGAAG′] |
| 2298 | NM_178151.2(DCX): c.683T > C | 1641 | DCX | [′CTGGAGACCGGGGTTGTCAAAAAACYCTAC |
| | (p.Leu228Pro) | | | ACTCTGGATGGAAAACAGGTA′] |
| 2299 | NM_178151.2(DCX): c.641T > C | 1641 | DCX | [′TCTTTTGAGCAAGTCCTCACTGATAYCACAG |
| | (p.Ile214Thr) | | | AAGCCATCAAACTGGAGACC′] |
| 2300 | NM_178151.2(DCX): c.412T > C | 1641 | DCX | [′AGACAACTTCTTTAAAAAGGTGGAGYACAC |
| | (p.Tyr138His) | | | CAAGAATGTCAATCCCAACTG′] |
| 2301 | NM_178151.2(DCX): c.272T > C | 1641 | DCX | [′TTGCTGGCTGACCTGACGCGATCTCYGTCTG |
| | (p.Leu91Pro) | | | ACAACATCAACCTGCCTCAG′] |
| 2302 | NM_178151.2(DCX): c.128T > C | 1641 | DCX | [′TGTAGCTTCTACCGAACCAGAACCTYGCAG |
| | (p.Leu43Ser) | | | GCACTGAGTAATGAGAAGAAA′] |
| 2303 | NM_178151.2(DCX): c.2T > C | 1641 | DCX | [′AGGTCTCTGAGGTTCCACCAAAATAYGGAA |
| | (p.Met1Thr) | | | CTTGATTTTGGACACTTTGAC′] |
| 2304 | NM_000252.2(MTM1): c.260T > | 4534 | MTM1 | [′TCTTCTCTAATACTTGATGTTCCTCYGGGTGT |
| | C (p.Leu87Pro) | | | GATCTCGAGAATTGAAAAA′] |
| 2305 | NM_000252.2(MTM1): c.683T > | 4534 | MTM1 | [′AACTTTCTGACTTAACCATAGGTGCYGTCAT |
| | C (p.Leu228Pro) | | | GGATTCATCCAGAAAATAAG′] |
| 2306 | NM_000252.2(MTM1): c.958T > | 4534 | MTM1 | [′TCATAATATTCATGTTATGCGGGAAYCTTTA |
| | C (p.Ser320Pro) | | | AAAAAAGTGAAGGACATTGT′] |
| 2307 | NM_000252.2(MTM1): | 4534 | MTM1 | [′TGTGTGTGGCAAATGTCAAAACAGGYAAGG |
| | c.1353 + 2T > C | | | AATATGAGGGATGAAAATACA′] |
| 2308 | NM_000252.2(MTM1): c.1367T > | 4534 | MTM1 | [′GTTTTTGTTTAGTTCCCTACAGCTTYTGAATT |
| | C (p.Phe456Ser) | | | CAATGAACAATTTTTGATT′] |
| 2309 | NM_000252.2(MTM1): c.1433T > | 4534 | MTM1 | [′CTGTATAGTTGCCGATTTGGTACTTYCTTATT |
| | C (p.Phe478Ser) | | | CAACTGTGAATCTGCTCGA′] |
| 2310 | NM_000252.2(MTM1): c.1495T > | 4534 | MTM1 | [′GGTTACAGAAAGGACTGTTTCTTTAYGGTCA |
| | C (p.Trp499Arg) | | | CTGATAAACAGTAATAAAGA′] |
| 2311 | NM_006579.2(EBP): c.310T > C | 10682 | EBP | [′TCTCTTCTTTTCTTCAGGGAAAGAGYATGCC |
| | (p.Tyr104His) | | | AAGGGAGACAGCCGATACAT′] |
| 2312 | NM_152296.4(ATP1A3): | 478 | ATP1A3 | [′GAGCCTGCCTGTGCCACAGGCCGCCYGATCT |
| | c.2270T > C (p.Leu757Pro) | | | TCGACAACCTAAAGAAGTCC′] |
| 2313 | NM_152296.4(ATP1A3): | 478 | ATP1A3 | [′GTGGCCCTGTCTCACATCGCTGGGCYCTGCA |
| | c.1250T > C (p.Leu417Pro) | | | ATCGCGCTGTCTTCAAGGGT′] |
| 2314 | NM_152296.4(ATP1A3): | 478 | ATP1A3 | [′GAACCGCATGACAGTCGCCCACATGYGGTT |
| | c.1144T > C (p.Trp382Arg) | | | TGACAACCAGATCCACGAGGC′] |
| 2315 | NM_152296.4(ATP1A3): | 478 | ATP1A3 | [′ATCTGCTCAGATAAGACAGGGACCCYCACT |
| | c.1112T > C (p.Leu371Pro) | | | CAGAACCGCATGACAGTCGCC′] |
| 2316 | NM_004963.3(GUCY2C): | −1 | — | [′CTTTCTGTCCTTCATTTCAGGTCCCYGTGCTG |
| | c.2782T > C (p.Cys928Arg) | | | CTGGAGTTGTGGGAATCAA′] |
| 2317 | NM_000109.3(DMD): c.1700T > | 1756 | DMD | [′GTTCACTGCATCTTCTTTTTCTGAARGCCATG |
| | C (p.Leu567Pro) | | | CACTAAAAAGGCACTGCAA′] |
| 2318 | NM_001543.4(NDST1): | 3340 | NDST1 | [′CAGCAACTACCCCAGCTCTGAGACCYTTGA |
| | c.1918T > C (p.Phe640Leu) | | | GGAGATCCAGTTTTTTAATGG′] |
| 2319 | NM_001999.3(FBN2): c.3740T > | 2201 | FBN2 | [′GGTGTCACAGCCTCCGTTCATTATCRTACAT |
| | C (p.Met1247Thr) | | | TCATCAATATCTGTGAAAAC′] |
| 2320 | NM_005154.4(USP8): c.2152T > | 9101 | USP8 | [′ACCTTCCAAACTGAAGCGCTCCTACYCCTCC |
| | C (p.Ser718Pro) | | | CCAGATATAACCCAGGCTAT′] |
| 2321 | NM_000734.3(CD247): c.2T > C | 919 | CD247 | [′CCTCTTTCTGAGGGAAAGGACAAGAYGAAG |
| | (p.Met1Thr) | | | TGGAAGGCGCTTTTCACCGCG′] |
| 2322 | NM_000663.4(ABAT): c.1433T > | 18 | ABAT | [′AAATCCATTCGTTTCCGTCCCACGCYGGTCT |
| | C (p.Leu478Pro) | | | TCAGGGATCACCACGCTCAC′] |
| 2323 | NM_005211.3(CSF1R): c.2717T > | 1436 | CSF1R | [′ACCCACAGACCCACCTTCCAGCAGAYCTGCT |
| | C (p.Ile906Thr) | | | CCTTCCTTCAGGAGCAGGCC′] |
| 2324 | NM_005211.3(CSF1R): c.2566T > | 1436 | CSFIR | [′CTTGGCCTTTGCAGGGCTGAATCCCYACCCT |
| | C (p.Tyr856His) | | | GGCATCCTGGTGAACAGCAA′] |
| 2325 | NM_005211.3(CSF1R): c.2480T > | 1436 | CSF1R | [′GTGAAGTGGATGGCCCCAGAGAGCAYCTTT |
| | C (p.Ile827Thr) | | | GACTGTGTCTACACGGTTCAG′] |
| 2326 | NM_005211.3(CSF1R): c.2450T > | 1436 | CSF1R | [′TGGGGACTGTCATCCCAGGCCCGCCYGCCTG |
| | C (p.Leu817Pro) | | | TGAAGTGGATGGCCCCAGAG′] |
| 2327 | NM_005211.3(CSF1R): c.1957T > | 1436 | CSF1R | [′GAACATCGTCAACCTTCTGGGAGCCYGTACC |
| | C (p.Cys653Arg) | | | CATGGAGGTAAGGGCCTTGG′] |
| 2328 | NM_005211.3(CSF1R): c.1745T > | 1436 | CSF1R | [′AAGTGGGAGTTCCCCCGGAACAACCYGCAG |
| | C (p.Leu582Pro) | | | TTTGGTGAGATGGCAGCTCAT′] |
| 2329 | NM_001563.3(IMPG1): c.461T > | 3617 | IMPG1 | [′AATTCCCAGGAGCACCTGGATCTTCYCCAGC |
| | C (p.Leu154Pro) | | | AGGTGAGCCTAAACACCACA′] |
| | NM_001130823.1(DNMT1): | 1786 | DNMT1 | [] |
| | c.1531T > C (p.Tyr511His) | | | |
| 2330 | NM_152515.4(CKAP2L): c.2T > C | 150468 | CKAP2L | [′AGCAGCGGTAGGCCCGGGCCCCACCRTGAC |
| | (p.Met1Thr) | | | TCTTCAGTGACAGTTTTTCTT′] |
| 2331 | NM_170707.3(LMNA): | 4000 | LMNA | [′GGCAACTCCAGCCCCCGAACCCAGGNGAGT |
| | c.1968 + 2T > C | | | TGTCTCTGCTTTGTCTCCAAA′] |
| 2332 | NM_153818.1(PEX10): c.890T > | 5192 | PEX10 | [′AGAAACCCCCTGTGCACCCTGTGCCYGGAG |
| | C (p.Leu297Pro) | | | GAGCGCAGGCACCCAACAGCC′] |
| 2333 | NM_153818.1(PEX10): c.2T > C | 5192 | PEX10 | [′TCGGGACCACCCGAACCCGCGGCCAYGGCC |
| | (p.Met1Thr) | | | CCGGCCGCCGCCAGCCCCCCG′] |
| 2334 | NM_014305.3(TGDS): c.700T > C | 23483 | TGDS | [′AATGCTTCTACAACATCAGTAGCATRAAGG |
| | (p.Tyr234His) | | | AAGTTTCTTGTTTGAAGCCCT′] |
| 2335 | NM_000256.3(MYBPC3): | 4607 | MYBPC3 | [′GTGAACCTTCTCATCCCTTTCCAGGYGGGAC |
| | c.2994 + 2T > C | | | TGGCCCCCTTCCCTGTCCCC′] |
| 2336 | NM_000256.3(MYBPC3): | 4607 | MYBPC3 | [′CTCATGTGCCCCCCCAGCCAGGCTCDCCCTG |
| | c.1624 + 2T > C | | | CACAATGAGCTCAGCCAGCG′] |
| 2337 | NM_000257.3(MYH7): c.2207T > | 4625 | MYH7 | [′GCGGCCATCCCTGAGGGACAGTTCAHTGAT |
| | C (p.Ile736Thr) | | | AGCAGGAAGGGGGCAGAGAAG′] |
| 2338 | NM_000138.4(FBN1): c.7754T > | 2200 | FBN1 | [′TGCCAGCATGGCTGCCAGAACATCAYTGGG |
| | C (p.Ile2585Thr) | | | GGCTACAGGTGCAGCTGCCCC′] |
| 2339 | NM_000371.3(TTR): c.190T > C | 7276 | TTR | [′GGCTGCTGATGACACCTGGGAGCCAYTTGC |
| | (p.Phe64Leu) | | | CTCTGGGTAAGTTGCCAAAGA′] |
| 2340 | NM_000126.3(ETFA): c.2T > C | 2108 | ETFA | [′GAGGTTGCGGCGGAAGCGGAGACCAYGTTC |
| | (p.Met1Thr) | | | CGAGCGGCGGCTCCGGGGCAG′] |
| 2341 | NM_006493.2(CLN5): c.2T > C | 1203 | CLN5 | [′GCGGGCCGGGCGCGGGGAGGTGTCAYGCGC |
| | (p.Met1Thr) | | | CGGAACCTGCGCTTGGGGCCA′] |
| 2342 | NM_000543.4(SMPD1): c.475T > | 6609 | SMPD1 | [′CTCAGTGCTGAGCCCATCTGAGGCCYGTGGC |
| | C (p.Cys159Arg) | | | CTGCTCCTGGGCTCCACCTG′] |
| 2343 | NM_000501.3(ELN): | 2006 | ELN | [′TTCCTGGAATTGGAGGCATCGCAGGYAACA |
| | c.889 + 2T > C | | | TCTGTCCCAGCAGGGGGCGGG′] |
| 2344 | NM_000169.2(GLA): c.758T > C | −1 | — | [′TGGACATCTTTTAACCAGGAGAGAAYTGTTG |
| | (p.Ile253Thr) | | | ATGTTGCTGGACCAGGGGGT′] |
| 2345 | NM_152743.3(BRAT1): c.176T > | 221927 | BRAT1 | [′GAGCACCCCTGCCTGGTGGAGCTGCYGTCCC |
| | C (p.Leu59Pro) | | | ATGTGCTGAAAGTCCAGGAC′] |
| 2346 | NM_000152.3(GAA): c.896T > C | 2548 | GAA | [′CTCTACGGGTCTCACCCTTTCTACCBGGCGC |
| | (p.Leu299Pro) | | | TGGAGGACGGCGGGTCGGCA′] |
| 2347 | NM_015384.4(NIPBL): c.7637T > | 25836 | NIPBL | [′AATGTGTCCCAGGGTATTTTATTACYTCTCA |
| | C (p.Leu2546Pro) | | | TGTTAAAACAACATTTGAAG′] |
| 2348 | NM_005641.3(TAF6): c.212T > C | 6878 | TAF6 | [′ATTCTTTAGCTTCAAGGCGTAGTCADTGTCA |
| | (p.Ile71Thr) | | | CTGGTGGTGAGCTTCTGCCG′] |
| 2349 | NM_170707.3(LMNA): c.710T > | 4000 | LMNA | [′ATTGACAATGGGAAGCAGCGTGAGTYTGAG |
| | C (p.Phe237Ser) | | | AGCCGGCTGGCGGATGCGCTG′] |
| 2350 | NM_000527.4(LDLR): c.1468T > | 3949 | LDLR | [′GGACTGGATCCACAGCAACATCTACYGGAC |
| | C (p.Trp490Arg) | | | CGACTCTGTCCTGGGCACTGT′] |
| 2351 | NM_000081.3(LYST): c.772T > C | 1130 | LYST | [′CATGAACAATTCTCCATTTGACTTAYGTCAT |
| | (p.Cys258Arg) | | | GTTTTGTTATCTTTATTAGA′] |
| 2352 | NM_000256.3(MYBPC3): | 4607 | MYBPC3 | [′CTTACAGGGCGAGGCACGGTGTGAGYGCCG |
| | c.3796T > C (p.Cys1266Arg) | | | CCTGGAGGTGCGAGGTGAGGA′] |
| 2353 | NM_000256.3(MYBPC3): | 4607 | MYBPC3 | [′TTCAGCAAGCAGGGAGTGTTGACTCYGGAG |
| | c.3713T > C (p.Leu1238Pro) | | | ATTAGAAAGCCCTGCCCCTTT′] |
| 2354 | NM_000256.3(MYBPC3): | 4607 | MYBPC3 | [′CAGAAAGCCGACAAGAAGACCATGGBGAGC |
| | c.3330 + 2T > C | | | CCAGGGTCTGGGGTCCCCACG′] |
| 2355 | NM_000256.3(MYBPC3): | 4607 | MYBPC3 | [′CGCAAAGGACCAGGCGGTGTTCAAAYGTGA |
| | c.1696T > C (p.Cys566Arg) | | | GGTCTCAGATGAGAATGTTCG′] |
| 2356 | NM_000256.3(MYBPC3): | 4607 | MYBPC3 | [′ATCGGAGGAGGGGGCGCAAGTCAAABGGTG |
| | c.1456T > C (p.Trp486Arg) | | | AGTTCCAGAAGCACGGGGCAT′] |
| 2357 | NM_000256.3(MYBPC3): | 4607 | MYBPC3 | [′TGCCCAGCCTGCCTTCACTGGCAGCYACCGC |
| | c.709T > C (p.Tyr237His) | | | TGTGAGGTGTCCACCAAGGA′] |
| 2358 | NM_000256.3(MYBPC3): | 4607 | MYBPC3 | [′GGAGCCCCCGATGACCCCATTGGCCYCTTCG |
| | c.467T > C (p.Leu156Pro) | | | TGATGCGGCCACAGGATGGC′] |
| 2359 | NM_000257.3(MYH7): c.2723T > | 4625 | MYH7 | [′GATGCTGAGGAGCGCTGTGATCAGCYGATC |
| | C (p.Leu908Pro) | | | AAAAACAAGATTCAGCTGGAG′] |
| 2360 | NM_000257.3(MYH7): c.2479T > | 4625 | MYH7 | [′TCGGGCCTTCATGGGGGTCAAGAATYGGCC |
| | C (p.Trp827Arg) | | | CTGGATGAAGCTCTACTTCAA′] |
| 2361 | NM_000257.3(MYH7): c.1400T > | 4625 | MYH7 | [′GTCCTGGACATCGCTGGCTTCGAGAYCTTCG |
| | C (p.Ile467Thr) | | | ATGTGAGTTGGGACCCCTGG′] |
| 2362 | NM_000257.3(MYH7): c.1048T > | 4625 | MYH7 | [′CACTTCAGAGGAGAAAAACTCCATGYATAA |
| | C (p.Tyr350His) | | | GCTGACAGGCGCCATCATGCA′] |
| 2363 | NM_000257.3(MYH7): c.730T > | 4625 | MYH7 | [′CGTCCGGAACGACAACTCCTCCCGCYTCGTG |
| | C (p.Phe244Leu) | | | AGTGGTCCCTGACCTTGGCC′] |
| 2364 | NM_005159.4(ACTC1): c.755T > | −1 | — | [′CTGCCTGATGGCCAAGTCATCACTAYTGGCA |
| | C (p.Ile252Thr) | | | ATGAGCGCTTCCGCTGTCCT′] |
| 2365 | NM_030662.3(MAP2K2): | 5605 | MAP2K2 | [′GCAGCAGAAGAAGCGGCTGGAAGCCNTTCT |
| | c.169T> C (p.Phe57Leu) | | | CACCCAGAAAGCCAAGGTCGG′] |
| 2366 | NM_000169.2(GLA): c.41T > C | −1 | — | [′CCAGAACTACATCTGGGCTGCGCGCYTGCG |
| | (p.Leu14Pro) | | | CTTCGCTTCCTGGCCCTCGTT′] |
| 2367 | NM_002294.2(LAMP2): | 3920 | LAMP2 | [′TATCTAGACTTTGTCTTTGCTGTGGYGAGTA |
| | c.864 + 2T > C | | | ACAGATTTTTTAAAGTTAGG′] |
| 2368 | NM_000551.3(VHL): c.227T > C | 7428 | VHL | [′TCGCGCGAGCCCTCCCAGGTCATCTYCTGCA |
| | (p.Phe76Ser) | | | ATCGCAGTCCGCGCGTCGTG′] |
| 2369 | NM_000551.3(VHL): c.266T > C | 7428 | VHL | [′CCGCGCGTCGTGCTGCCCGTATGGCHCAACT |
| | (p.Leu89Pro) | | | TCGACGGCGAGCCGCAGCCC′] |
| 2370 | NM_000551.3(VHL): c.473T > C | 7428 | VHL | [′GTTTTTGCCCTTCCAGTGTATACTCHGAAAG |
| | (p.Leu158Pro) | | | AGCGATGCCTCCAGGTTGTC′] |
| 2371 | NM_000051.3(ATM): | −1 | — | [′GCAGTGTAGCTACTTCTTCTATTGGYAATCT |
| | c.8584 + 2T > C | | | TCTTGTACATATAGTAGATT′] |
| 2372 | NM_058216.2(RAD51C): | 5889 | RAD51C | [′AGGTGTTGGAAAAACACAATTATGGYAAAA |
| | c.404 + 2T > C | | | TAAAGTGTTCTCCTTTTAAGG′] |
| 2373 | NM_000455.4(STK11): c.545T > | 6794 | STK11 | [′CACAAGGACATCAAGCCGGGGAACCYGCTG |
| | C (p.Leu182Pro) | | | CTCACCACCGGTGGCACCCTC′] |
| 2374 | NM_001199107.1(TBC1D24): | 57465 | TBC1D24 | [′GGACAACACGCAGGTGCCCAGCTACYGCCT |
| | c.313T > C (p.Cys105Arg) | | | GAATGCACGCGGCGAGGGGGC′] |
| 2375 | NM_000899.4(KITLG): c.98T > C | 4254 | KITLG | [′ACTGAAGGGATCTGCAGGAATCGTGYGACT |
| | (p.Val33Ala) | | | AATAATGTAAAAGACGTCACT′] |
| 2376 | NM_001195129.1(PRSS56): | 646960 | PRSS56 | [′GCAGTGCCTGCAGCGCCGGCGGCGAYGCGG |
| | c.1183T > C (p.Cys395Arg) | | | TCAGTTCTGTTCACCCGGACC′] |
| 2377 | NM_006888.4(CALM1): c.268T > | 801 | CALM1 | [′TAGTGAAGAAGAAATCCGTGAGGCAYTCCG |
| | C (p.Phe90Leu) | | | AGTCTTTGACAAGGTAATCCA′] |
| 2378 | NM_017534.5(MYH2): c.5609T > | −1 | — | [′GAAGATAGAAAGAATATTCTCAGGCYTCAA |
| | C (p.Leu1870Pro) | | | GATTTGGTAGATAAACTTCAG′] |
| 2379 | NM_003000.2(SDHB): c.574T > C | 6390 | SDHB | [′CTACGAGTGCATTCTCTGTGCCTGCYGTAGC |
| | (p.Cys192Arg) | | | ACCAGCTGCCCCAGCTACTG′] |
| 2380 | NM_000314.6(PTEN): c.406T > C | 5728 | PTEN | [′AAAGGGACGAACTGGTGTAATGATAYGTGC |
| | (p.Cys136Arg) | | | ATATTTATTACATCGGGGCAA′] |
| 2381 | NM_000051.3(ATM): c.2T > C | 472 | ATM | [′ATGTGTGTTCTGAAATTGTGAACCAYGAGTC |
| | (p.Met1Thr) | | | TAGTACTTAATGATCTGCTT′] |
| 2382 | NM_052867.2(NALCN): | 259232 | NALCN | [′GGTCCTGGAAAAAAGCTTGGGAGTTYGGTT |
| | c.1526T > C (p.Leu509Ser) | | | GTATTTACTGCCAGCCTCTTG′] |
| 2383 | NM_000314.6(PTEN): c.545T > C | 5728 | PTEN | [′TATGTGTATTATTATAGCTACCTGTYAAAGA |
| | (p.Leu182Ser) | | | ATCATCTGGATTATAGACCA′] |
| 2384 | NM_005957.4(MTHFR): | 4524 | MTHFR | [′ACGCAGGGCGTCAGGACGCAGGGTCRTGGA |
| | c.1969T > C (p.Ter657Arg) | | | GCCTCCGTTTCTCTCGCATTC′] |
| 2385 | NM_005957.4(MTHFR): | 4524 | MTHFR | [′AACCTGGTGGACAATGACTTCCCACYGGAC |
| | c.1883T > C (p.Leu628Pro) | | | AACTGCCTCTGGCAGGTGGTG′] |
| 2386 | NM_005957.4(MTHFR): | 4524 | MTHFR | [′CTGTGGATTGAGCGGTGGGGAAAGCYGTAT |
| | c.1793T > C (p.Leu598Pro) | | | GAGGAGGAGTCCCCGTCCCGC′] |
| 2387 | NM_005957.4(MTHFR): | 4524 | MTHFR | [′AGCGGGGGCTATGTCTTCCAGAAGGYGTGG |
| | c.1530 + 2T > C | | | TAGGGAGGCACGGGGTGCCCC′] |
| 2388 | NM_005957.4(MTHFR): c.388T > | 4524 | MTHFR | [′CCTGGAGACCATCCTGCACATGACCYGCTGC |
| | C (p.Cys130Arg) | | | CGTCAGCGCCTGGAGGAGAT′] |
| 2389 | NM_000264.3(PTCH1): | 5727 | PTCH1 | [′AACCCCTGGACGGCCGGGATCATTGYGAGT |
| | c.3168 + 2T > C | | | GTATTATAAGGGGCTTTGTGG′] |
| 2390 | NM_000030.2(AGXT): c.302T > C | 189 | AGXT | [′GTGCTGGAGCCTGGGGACTCCTTCCYGGTTG |
| | (p.Leu101Pro) | | | GGGCCAATGGCATTTGGGGG′] |
| 2391 | NM_000030.2(AGXT): c.322T > C | 189 | AGXT | [′CTTCCTGGTTGGGGCCAATGGCATTYGGGGG |
| | (p.Trp108Arg) | | | CAGCGAGCCGTGGACATCGG′] |
| 2392 | NM_000023.2(SGCA): c.371T > C | 6442 | SGCA | [′ACTCGGCAGAGGCTGGTGCTGGAGAYTGGG |
| | (p.Ile124Thr) | | | GACCCAGAAGGTACCTCTAGC′] |
| 2393 | NM_016035.4(COQ4): c.155T > C | 51117 | COQ4 | [′CGCCATCGCCGCGGAGCCGGCGGCCRACAG |
| | (p.Leu52Ser) | | | CCCTTTCTGCAGCGGGGAGGT′] |
| 2394 | NM_000510.2(FSHB): c.298T > C | 2488 | FSHB | [′GTATACATACCCAGTGGCCACCCAGYGTCA |
| | (p.Cys100Arg) | | | CTGTGGCAAGTGTGACAGCGA′] |
| 2395 | NM_007374.2(SIX6): c.110T > C | 4990 | SIX6 | [′CGCCTGGGTCGCTTCCTCTGGTCGCYGCCCG |
| | (p.Leu37Pro) | | | TGGCCCCTGCGGCCTGCGAG′] |
| 2396 | NM_000314.6(PTEN): c.202T > C | 5728 | PTEN | [′AAAGCATAAAAACCATTACAAGATAHACAA |
| | (p.Tyr68His) | | | TCTGTAAGTATGTTTTCTTAT′] |
| 2397 | NM_001103.3(ACTN2): c.683T > | 88 | ACTN2 | [′GAGAAGCACCTGGATATTCCTAAAAYGTTG |
| | C (p.Met228Thr) | | | GATGCTGAAGGTGAGATGAAA′] |
| 2398 | NM_005249.4(FOXG1): c.700T > | 2290 | FOXG1 | [′GCAGAACTCCATCCGCCACAATCTGYCCCTC |
| | C (p.Ser234Pro) | | | AACAAGTGCTTCGTGAAGGT′] |
| 2399 | NM_001165963.1(SCN1A): | −1 | — | [′ATTCCATCCATCATGAATGTGCTTCYGGTTT |
| | c.4055T > C (p.Leul352Pro) | | | GTCTTATATTCTGGCTAATT′] |
| 2400 | NM_001165963.1(SCN1A): | 6323 | SCN1A | [′GCTCTGGGAAATTTAACCCTCGTCTYGGCCA |
| | c.2690T > C (p.Leu897Ser) | | | TCATCGTCTTCATTTTTGCC′] |
| 2401 | NM_001165963.1(SCN1A): | 6323 | SCN1A | [′CTAATAAATTTGATCCTGGCTGTGGHGGCCA |
| | c.1265T > C (p.Val422Ala) | | | TGGCCTACGAGGAACAGAAT′] |
| 2402 | NM_001165963.1(SCN1A): | 6323 | SCN1A | [′TTTCTTTTACCCCACTTGCAGCCAAYGTCCA |
| | c.1033T > C (p.Cys345Arg) | | | GAGGGATATATGTGTGTGAA′] |
| 2403 | NM_001165963.1(SCN1A): | 6323 | SCN1A | [′AGATGTAATGATCCTGACTGTGTTCYGTCTG |
| | c.769T > C (p.Cys257Arg) | | | AGCGTATTTGCTCTAATTGG′] |
| 2404 | NM_001165963.1(SCN1A): | 6323 | SCN1A | [′TTCCGGTTCAGTGCCACCTCTGCCCYGTACA |
| | c.323T > C (p.Leu108Pro) | | | TTTTAACTCCCTTCAATCCT′] |
| 2405 | NM_000426.3(LAMA2): | 3908 | LAMA2 | [′GCAGAATCAGAACCAGCTCTTTTGAYAGGG |
| | c.8282T > C (p.Ile2761Thr) | | | AGCAAGCAGTTCGGGCTTTCA′] |
| 2406 | NM_001199.3(BMP1): | 649 | BMP1 | [′GTCTGTGACATTTCCTGTTGTGAAGYAAAAG |
| | c.*241T > C | | | AGGGACCCCTGCGTCCTGCT′] |
| 2407 | NM_001987.4(ETV6): c.1046T > | 2120 | ETV6 | [′CTTTGGGATTACGTCTATCAGTTGCYTTCTG |
| | C (p.Leu349Pro) | | | ACAGCCGGTACGAAAACTTC′] |
| 2408 | NM_004974.3(KCNA2): c.788T > | 3737 | KCNA2 | [′ATGAACATCATTGACATTGTGGCCAYCATCC |
| | C (p.Ile263Thr) | | | CCTACTTCATCACCCTGGGG′] |
| 2409 | NM_002055.4(GFAP): c.791T > C | 2670 | GFAP | [′GTGTCCCCCACCTAGTTTGCAGACCYGACAG |
| | (p.Leu264Pro) | | | ACGCTGCTGCCCGCAACGCG′] |
| 2410 | NM_002055.4(GFAP): c.239T > C | 2670 | GFAP | [′GAGATGATGGAGCTCAATGACCGCTYTGCC |
| | (p.Phe80Ser) | | | AGCTACATCGAGAAGGTTCGC′] |
| 2411 | NM_002055.4(GFAP): c.218T > C | 2670 | GFAP | [′ACCCGGGCCAGTGAGCGGGCAGAGADGATG |
| | (p.Met73Thr) | | | GAGCTCAATGACCGCTTTGCC′] |
| 2412 | NM_001288953.1(TTC7A): c.191 | 57217 | TTC7A | [′TTTGCCTGATGCCCATGATGCAGACYCTGGT |
| | 2T > C (p.Ser638Pro) | | | AAGAACGAGCTCCTTGGGCC′] |
| 2413 | NM_000257.3(MYH7): c.5117T > | −1 | — | [′TCCCGGAAGCTGGCGGAGCAGGAGCYGATT |
| | C (p.Leu1706Pro) | | | GAGACTAGTGAGCGGGTGCAG′] |
| 2414 | NM_000257.3(MYH7): c.4772T > | −1 | — | [′ATGGAACAGGCCAAGCGCAACCACCWGCG |
| | C (p.Leu1591Pro) | | | GGTGGTGGACTCGCTGCAGACC′] |
| 2415 | NM_016218.2(POLK): c.609T > C | 51426 | POLK | [′TGAGTCTTGATGAAGCCTACTTGAAYATAAC |
| | (p.Asn203=) | | | AAAGCACTTAGAAGAAAGAC′] |
| 2416 | NM_016218.2(POLK): c.*66T > C | 51426 | POLK | [′TATTTTATAATCAATGAATTTGTTCYTTCTGA |
| | | | | TTTTAAGTTTGCAGATTTA′] |
| 2417 | NM_015662.2(IFT172): c.770T > | 26160 | IFT172 | [′AGTCCTGGGGGCCAGTCTGTTGTGCYAGGA |
| | C (p.Leu257Pro) | | | AGTTATGACAGGTAAGTCCCC′] |
| 2418 | NM_178454.4(DRAM2): c.79T > | 128338 | DRAM2 | [′GACATCTGCTGCTTTCATATTTTCAYACATT |
| | C (p.Tyr27His) | | | ACTGCAGTAACACTCCACCA′] |
| 2419 | NM_032790.3(ORAI1): c.581T > | 84876 | ORAI1 | [′CTCTTCCTAGCTGAGGTGGTGCTGCYCTGCT |
| | C (p.Leu194Pro) | | | GGGTCAAGTTCTTGCCCCTC′] |
| 2420 | NM_001135669.1(XPR1): | 9213 | XPR1 | [′GAGTTCTACCTCAGTCTAATCCTGCYGCAGA |
| | c.434T > C (p.Leu145Pro) | | | ACTATCAGGTACTTAGATTC′] |
| 2421 | NM_001135669.1(XPR1): | 9213 | XPR1 | [′AAACTGGCCTTCAGTGAGTTCTACCYCAGTC |
| | c.419T > C (p.Leu140Pro) | | | TAATCCTGCTGCAGAACTAT′] |
| 2422 | NM_001135669.1(XPR1): | 9213 | XPR1 | [′GACAGACAAAAGGCTATGAAGCGTTYACGT |
| | c.653T > C (p.Leu218Ser) | | | GTCCCCCCTTTGGGAGCTGCT′] |
| 2423 | NM_005859.4(PURA): c.218T > C | 5813 | PURA | [′CGGGTGGACATCCAGAACAAGCGCTYCTAC |
| | (p.Phe73Ser) | | | CTGGACGTGAAGCAGAACGCC′] |
| 2424 | NM_005859.4(PURA): c.563T > C | 5813 | PURA | [′CTGGGCTCCACGCAGGGCCAGACCAYTGCG |
| | (p.Ile188Thr) | | | CTGCCCGCGCAGGGGCTCATC′] |
| 2425 | NM_001399.4(EDA): | 1896 | EDA | [′TCTGACTCCCAGGACGGGCACCAGGKGAGT |
| | c.396 + 2T > C | | | CACCTAGTAGGGGCGGCGGCG′] |
| 2426 | NM_001848.2(COL6A1): | 1291 | COL6A1 | [′TCCAGGGGACCCAAGGGCTACAAGGYGAGC |
| | c.957 + 2T > C | | | GTGGGCTGCTGGGAGGGGGGA′] |
| 2427 | NM_000454.4(SOD1): c.341T > C | 6647 | SOD1 | [′TCACTCTCAGGAGACCATTGCATCAYTGGCC |
| | (p.Ile114Thr) | | | GCACACTGGTGGTAAGTTTT′] |
| 2428 | NM_020451.2(SEPN1): | 57190 | SEPN1 | [′CTTCTACTACACTGTGATGTTCCGGYGAGTG |
| | c.872 + 2T > C | | | GGCCACACTGGCTGGCCTGG′] |
| 2429 | NM_004453.3(ETFDH): | 2110 | ETFDH | [′GGTCTAGACTATCAGAATCCATACCYGAGTC |
| | c.1001T > C (p.Leu334Pro) | | | CATTTAGAGAGTTCCAAAGG′] |
| 2430 | NM_198056.2(SCN5A): | 6331 | SCN5A | [′CAGCTGTGGACTCCAGGGGGGTAGGYTGCC |
| | c.4299 + 6T > C | | | ACAGTGGCTTCTTCCACCAAG′] |
| 2431 | NM_198056.2(SCN5A): | 6331 | SCN5A | [′TTCACAGGGATTTTCACAGCAGAGABGACCT |
| | c.2291T > C (p.Met764Thr) | | | TCAAGATCATTGCCCTCGAC′] |
| 2432 | NM_198056.2(SCN5A): | 6331 | SCN5A | [′AGAGTTAGAGGAGTCTCGCCACAAGBGTCC |
| | c.2047T > C (p.Cys683Arg) | | | ACCATGCTGGAACCGTCTCGC′] |
| 2433 | NM_004415.2(DSP): c.4961T > C | 1832 | DSP | [′GAAAAGCAGAGGACCCAGGAAGAGCYGAG |
| | (p.Leu1654Pro) | | | GAGGCTCTCTTCTGAGGTCGAG′] |
| 2434 | NM_000238.3(KCNH2): | 3757 | KCNH2 | [′CGGGGCGACGTCGTCGTGGCCATCCYGGGT |
| | c.2396T > C (p.Leu799Pro) | | | ATGGGGTGGGGGGCGGGCACT′] |
| 2435 | NM_000238.3(KCNH2): | 3757 | KCNH2 | [′TTCATCTCCCGGGGCTCCATCGAGAYCCTGC |
| | c.2366T > C (p.Ile789Thr) | | | GGGGCGACGTCGTCGTGGCC′] |
| 2436 | NM_000238.3(KCNH2): | 3757 | KCNH2 | [′CTGCGTCATGCTCATTGGCTGTGAGYGTGCC |
| | c.1945 + 6T > C | | | CAGGGGCGGGCGGCGGGGAG′] |
| 2437 | NM_000238.3(KCNH2): | 3757 | KCNH2 | [′TCCCAACACCAACTCAGAGAAGATCBTCTCC |
| | c.1918T > C (p.Phe640Leu) | | | ATCTGCGTCATGCTCATTGG′] |
| 2438 | NM_000238.3(KCNH2): | 3757 | KCNH2 | [′CTACACGGCTGTCTTCACACCCTACYCGGCT |
| | c.1282T > C (p.Ser428Pro) | | | GCCTTCCTGCTGAAGGAGAC′] |
| 2439 | NM_000238.3(KCNH2): c.125T > | 3757 | KCNH2 | [′GCTCGGGTGGAGAACTGCGCCGTCAHCTAC |
| | C (p.Ile42Thr) | | | TGCAACGACGGCTTCTGCGAG′] |
| 2440 | NM_000218.2(KCNQ1): | 3784 | KCNQ1 | [′GTCTTTGCCATCTCCTTCTTTGCGCYCCCAGC |
| | c.1025T > C (p.Leu342Pro) | | | GGTAGGTGCCCCGTGGGTG′] |
| 2441 | NM_000218.2(KCNQ1): | 3784 | KCNQ1 | [′CCCAAACCCAAGAAGTCTGTGGTGGYGAGT |
| | c.1251 + 2T > C | | | AGCCCACCTGCCACCAGGGCA′] |
| 2442 | NM_130799.2(MEN1): c.547T > C | 4221 | MEN1 | [′CCTCGCCCTGTCTGAGGATCATGCCYGGGTA |
| | (p.Trp183Arg) | | | GTGTTTGGGCCCAATGGGGA′] |
| 2443 | NM_000138.4(FBN1): c.7531T > | 2200 | FBN1 | [′CACCATTGGCGGCTTCACATGCAAAYGTCCT |
| | C (p.Cys2511Arg) | | | CCCGGATTTACCCAACACCA′] |
| 2444 | NM_000138.4(FBN1): c.7111T > | 2200 | FBN1 | [′ATGCTGCTGTGACGGAGGGAGAGGCYGGGG |
| | C (p.Trp2371Arg) | | | TCCCCACTGTGAGATCTGCCC′] |
| 2445 | NM_000138.4(FBN1): c.6274T > | 2200 | FBN1 | [′CTGCTGTGCCTTGAAGGGAGAAGGCYGGGG |
| | C (p.Trp2092Arg) | | | AGACCCCTGCGAGCTCTGCCC′] |
| 2446 | NM_000138.4(FBN1): c.5746T > | 2200 | FBN1 | [′CACAATTGGTTCCTTCAACTGCCGCYGCAAT |
| | C (p.Cys1916Arg) | | | CATGGTTTCATCCTTTCTCA′] |
| 2447 | NM_000138.4(FBN1): c.5726T > | 2200 | FBN1 | [′GGGAATGGAACTTGCCGGAACACAAYTGGT |
| | C (p.Ile1909Thr) | | | TCCTTCAACTGCCGCTGCAAT′] |
| 2448 | NM_000138.4(FBN1): c.1468 + 2T | 2200 | FBN1 | [′TGGACCTCCGTGGGGAGTGTATTGGYACGT |
| | > C | | | GATCCATCCTAGGTTGGCACC′] |
| 2449 | NM_004572.3(PKP2): c.2386T > | 5318 | PKP2 | [′TCTCATTGAAACTACAGCCTCTGCCYGTTAC |
| | C (p.Cys796Arg) | | | ACATTGAACAACATAATCCA′] |
| 2450 | NM_004333.4(BRAF): c.1783T > | 673 | BRAF | [′AGACCTCACAGTAAAAATAGGTGATYTTGG |
| | C (p.Phe595Leu) | | | TCTAGCTACAGTGAAATCTCG′] |
| 2451 | NM_000543.4(SMPD1): c.416T > | 6609 | SMPD1 | [′GCCGTGTGCCAATCCATTGTCCACCYCTTTG |
| | C (p.Leu139Pro) | | | AGGATGACATGGTGGAGGTG′] |
| 2452 | NM_000016.5(ACADM): | 34 | ACADM | [′GAAGCAGATACCCCAGGAATTCAGAHTGGG |
| | c.698T > C (p.Ile233Thr) | | | AGAAAGGTAAAGTATTTATTA′] |
| 2453 | NM_004453.3(ETFDH): | 2110 | ETFDH | [′AGGAGGACCTGCTTACAATGGAATGYAAAC |
| | c.1852T > C (p.Ter618Gln) | | | TGCAGCTAGCCAGTTTCTTTC′] |
| 2454 | NM_000255.3(MUT): c.842T > C | 4594 | MUT | [′GCCATTCTGGAGCTGGCCTATACTTYAGCAG |
| | (p.Leu281Ser) | | | ATGGATTGGAGTACTCTAGA′] |
| 2455 | NM_000277.1(PAH): c.2T > C | 5053 | PAH | [′AGACCTCACTCCCGGGGAGCCAGCABGTCC |
| | (p.Met1Thr) | | | ACTGCGGTCCTGGAAAACCCA′] |
| 2456 | NM_000017.3(ACADS): | 35 | ACADS | [′GGCAGCCATGGCCAAGCTGGCCGCCYCGGA |
| | c.1057T > C (p.Ser353Pro) | | | GGCCGCGACCGCCATCAGCCA′] |
| 2457 | NM_002225.3(IVD): | 3712 | IVD | [′GCGTATTGGGCATCACAGCCCCTGGYGAGT |
| | c.295 + 2T > C | | | ATAGTGTCTTTCCCTAAAAAG′] |
| 2458 | NM_000030.2(AGXT): c.2T > C | 189 | AGXT | [′CGAGCGGCAGGTTGGGTGCGGACCAYGGCC |
| | (p.Met1Thr) | | | TCTCACAAGCTGCTGGTGACC′] |
| 2459 | NM_000030.2(AGXT): c.77T > C | 189 | AGXT | [′CTCTCCATCCCCAACCAGCTCCTGCYGGGGC |
| | (p.Leu26Pro) | | | CTGGTCCTTCCAACCTGCCT′] |
| 2460 | NM_000030.2(AGXT): c.449T > C | 189 | AGXT | [′GGCCTGGCCCAGCACAAGCCAGTGCYGCTG |
| | (p.Leu150Pro) | | | TTCTTAACCCACGGGGAGTCG′] |
| 2461 | NM_000030.2(AGXT): c.497T > C | 189 | AGXT | [′TCGTCCACCGGCGTGCTGCAGCCCCHTGATG |
| | (p.Leu166Pro) | | | GCTTCGGGGAACTCTGCCAC′] |
| 2462 | NM_000030.2(AGXT): c.661T > C | 189 | AGXT | [′CGCCCCTCCAGGGACCTCGCTCATCYCCTTC |
| | (p.Ser221Pro) | | | AGTGACAAGGCCAAGTGAGT′] |
| 2463 | NM_000030.2(AGXT): c.757T > C | 189 | AGXT | [′CAAGTGGCTGGCCAACTTCTGGGGCYGTGA |
| | (p.Cys253Arg) | | | CGACCAGCCCAGGATGTGAGG′] |
| 2464 | NM_000030.2(AGXT): c.806T > C | 189 | AGXT | [′CATCACACAATCCCCGTCATCAGCCYGTACA |
| | (p.Leu269Pro) | | | GCCTGAGAGAGAGCCTGGCC′] |
| 2465 | NM_000030.2(AGXT): c.851T > C | 189 | AGXT | [′CCAGCGCCATCTCCCACACAGGGCCYGGAG |
| | (p.Leu284Pro) | | | AACAGCTGGCGCCAGCACCGC′] |
| 2466 | NM_000030.2(AGXT): c.893T > C | 189 | AGXT | [′CAGCACCGCGAGGCCGCGGCGTATCYGCAT |
| | (p.Leu298Pro) | | | GGGCGCCTGCAGGCACTGGGG′] |
| 2467 | NM_000030.2(AGXT): c.947T > C | 189 | AGXT | [′TGAGCCAGGCCCCTCCTGCAGGCGCYCCGG |
| | (p.Leu316Pro) | | | CTTCCCACAGTCACCACTGTG′] |
| 2468 | NM_000030.2(AGXT): c.1076T > | 189 | AGXT | [′AGCCCGCCCTGTGCCCCCCAGGTGCYGCGG |
| | C (p.Leu359Pro) | | | ATCGGCCTGCTGGGCTGCAAT′] |
| 2469 | NM_000030.2(AGXT): c.1151T > | 189 | AGXT | [′GTGACGGAGGCCCTGAGGGCGGCCCYGCAG |
| | C (p.Leu384Pro) | | | CACTGCCCCAAGAAGAAGCTG′] |
| 2470 | NM_012203.1(GRHPR): c.203T > | 9380 | GRHPR | [′TCCGACCACGTGGACAAGAGGATCCYGGAT |
| | C (p.Leu68Pro) | | | GCTGCAGGTGCACACTGGGTG′] |
| 2471 | NM_012203.1(GRHPR): c.965T > | 9380 | GRHPR | [′CTGGCTGGCCTGAGAGGGGAGCCGABGCCT |
| | C (p.Met322Thr) | | | AGTGAACTCAAGCTGTAGCCA′] |
| 2472 | NM_138413.3(HOGA1): c.533T > | 112817 | HOGA1 | [′GTCCCAGCCAACACAGGGCTGGACCYGCCT |
| | C (p.Leu178Pro) | | | GTGGATGCAGTGGTCACGCTT′] |
| 2473 | NM_138413.3(HOGA1): c.875T > | 112817 | HOGA1 | [′GGGATCCCAGGGCTGAAGAAAATCAYGGAC |
| | C (p.Met292Thr) | | | TGGTTTGGCTACTATGGAGGC′] |
| 2474 | NM_004370.5(COL12A1): | 1303 | COL12A1 | [′TTCTTGACTGATGCCTCCTGGAGCAYTGGGG |
| | 1c.700T > C (p.Ile2334Thr) | | | ACGATAATTTTAACAAAGTT′] |
| 2475 | NM_019074.3(DLL4): c.583T > C | 54567 | DLL4 | [′CCTGTGCAAGAAGCGCAATGACCACYTCGG |
| | (p.Phe195Leu) | | | CCACTATGTGTGCCAGCCAGA′] |
| 2476 | NM_019074.3(DLL4): c.1168T > | 54567 | DLL4 | [′CCAGGGGGCCAACTATGCTTGTGAAYGTCC |
| | C (p.Cys390Arg) | | | CCCCAACTTCACCGGCTCCAA′] |
| 2477 | NM_015909.3(NBAS): c.3164T > | 51594 | NBAS | [′TCAGAGCTTTTGGAAAAACATGGACYCGAG |
| | C (p.Leu1055Pro) | | | AAACCAATTTCATTTGTTAAA′] |
| 2478 | NM_000263.3(NAGLU): | 4669 | NAGLU | [′GCCTCCTTCCAGGGCCAGCCCTTCAYCTGGT |
| | c.1208T > C (p.Ile403Thr) | | | GCATGCTGCACAACTTTGGG′] |
| 2479 | NM_203290.2(POLRIC): | 9533 | POLRIC | [′TCTACAGTTTCGTCTCCAGGTCAGAYGCACT |
| | c.436T > C (p.Cys146Arg) | | | CGGAACCCCCATGCTGCTAA′] |
| 2480 | NM_018359.3(UFSP2): c.868T > | −1 | — | [′CCAGGGCATATATGGCTATCATCATYATATG |
| | C (p.Tyr290His) | | | CAGGATCGCATAGATGACAA′] |
| 2481 | NM_000310.3(PPT1): c.2T > C | 5538 | PPT1 | [′CCGCGGTCATGTGACACAGCGAAGAYGGCG |
| | (p.Met1Thr) | | | TCGCCCGGCTGCCTGTGGCTC′] |
| 2482 | NM_021007.2(SCN2A): | 6326 | SCN2A | [′CTAATAAATTTGATCTTGGCTGTGGYGGCCA |
| | c.1271T > C (p. Val424Ala) | | | TGGCCTATGAGGAACAGAAT′] |
| 2483 | NM_021007.2(SCN2A): | 6326 | SCN2A | [′GTTGACCTGGCCATCACCATCTGCAYTGTCT |
| | c.2306T > C (p.Ile769Thr) | | | TAAATACACTCTTCATGGCT′] |
| 2484 | NM_021007.2(SCN2A): | 6326 | SCN2A | [′TATGCAGCTGTTGATTCACGAAATGYAAGTC |
| | c.4308 + 2T > C | | | TAGTTAGAGGGAAATTGTTT′] |
| 2485 | NM_021007.2(SCN2A): | 6326 | SCN2A | [′TGGATTAATCTGGTGTTTATTGTTCYGTTCA |
| | c.4718T > C (p.Leu1573Pro) | | | CTGGAGAATGTGTGCTGAAA′] |
| 2486 | NM_001165963.1(SCN1A): | −1 | — | [′CAGCTCATTGCCATGGATTTGCCCAHGGTGA |
| | c.5567T > C (p.Met1856Thr) | | | GTGGTGACCGGATCCACTGT′] |
| 2487 | NM_001165963.1(SCN1A): | 6323 | SCN1A | [′AATCCCAATTATGGCTACACAAGCTYTGATA |
| | c.1094T > C (p.Phe365Ser) | | | CCTTCAGTTGGGCTTTTTTG′] |
| 2488 | NM_001165963.1(SCN1A): | 6323 | SCN1A | [′TCGGCATTGAGAACATTCAGAGTTCYCCGA |
| | c.662T > C (p.Leu221Pro) | | | GCATTGAAGACGATTTCAGTC′] |
| 2489 | NM_000806.5(GABRA1): | 2554 | GABRA1 | [′ATTCAAACATACCTGCCATGCATAAYGACA |
| | c.788T > C (p.Met263Thr) | | | GTGATTCTCTCACAAGTCTCC′] |
| 2490 | NM_014191.3(SCN8A): | 6334 | SCN8A | [′GCCCGTATTGGGCGCATCTTGCGTCYGATCA |
| | c.4889T > C (p.Leu1630Pro) | | | AAGGCGCCAAAGGGATTCGT′] |
| 2491 | NM_005249.4(FOXG1): c.673T > | 2290 | FOXG1 | [′TTACTACCGCGAGAACAAGCAGGGCYGGCA |
| | C (p.Trp225Arg) | | | GAACTCCATCCGCCACAATCT′] |
| 2492 | NM_017882.2(CLN6): | 54982 | CLN6 | [′AAGAATCTCAAGCCGGAGACGCTGGYGAGG |
| | c.486 + 2T > C | | | CCACCTCCTGCTCCCTGCCTG′] |
| 2493 | NM_002693.2(POLG): c.1808T > | 5428 | POLG | [′AGGGAAGCCATCCCAGGTAAGTGCCRTGAG |
| | C (p.Met603Thr) | | | TTTAGGTGTGACCCGCATCTG′] |
| 2494 | NM_002693.2(POLG): c.1283T > | 5428 | POLG | [′AGGCAGGTAGGAGACACCCATCTCCRGCAT |
| | C (p.Leu428Pro) | | | GCCGGCCAGAGTCACTGGGTG′] |
| 2495 | NM_018129.3(PNPO): c.2T > C | 55163 | PNPO | [′GGGTCACGTGGCCGGCGGCCCCCCAYGACG |
| | (p.Met1Thr) | | | TGCTGGCTGCGGGGCGTCACG′] |
| 2496 | NM_007254.3(PNKP): | 11284 | PNKP | [′ACCCCCACCCCCGCCCCAGGGCCTCRCCGGA |
| | c.1029 + 2T > C | | | TCAAAGGCTGGGAGCTCGAA′] |
| 2497 | NM_172107.2(KCNQ2): c.583T > | 3785 | KCNQ2 | [′CTCCCAGGGCAACGTCTTTGCCACAYCTGCG |
| | C (p.Ser195Pro) | | | CTCCGGAGCCTGCGCTTCCT′] |
| 2498 | NM_000026.2(ADSL): c.340T > C | 158 | ADSL | [′TATTCACCTTGGTGCTACTTCTTGCYATGTTG |
| | (p.Tyr114His) | | | GAGACAATACTGTAGGCGC′] |
| 2499 | NM_000026.2(ADSL): c.1339T > | 158 | ADSL | [′CCAGTTGGATCATTTACTGGATCCTYCTTCT |
| | C (p.Ser447Pro) | | | TTCACTGGTCGTGCCTCCCA′] |
| 2500 | NM_001356.4(DDX3X): c.704T > | 1654 | DDX3X | [′GGGTCTGGAAAAACTGCAGCATTTCYGTTGC |
| | C (p.Leu235Pro) | | | CCATCTTGAGTCAGATTTAT′] |
| 2501 | NM_001356.4(DDX3X): | 1654 | DDX3X | [′TAAAAATTTTTTTTCTTTCAGATGCYGGCTC |
| | c.1175T > C (p.Leu392Pro) | | | GTGATTTCTTAGATGAATAT′] |
| 2502 | NM_001356.4(DDX3X): | 1654 | DDX3X | [′GACATTTCAAATGTGAAACATGTTAYCAATT |
| | c.1541T > C (p.Ile514Thr) | | | TTGACTTGCCAAGTGATATT′] |
| 2503 | NM_020533.2(MCOLN1): | 57192 | MCOLN1 | [′ACCATCGCCTTCCGACACCTCTTCCYGCTGG |
| | c.317T > C (p.Leu106Pro) | | | GCTACTCGGACGGAGCGGAT′] |
| 2504 | NM_020533.2(MCOLN1): | 57192 | MCOLNI | [′TACTGCTTCTGTGGCTGGATCGTGCYGGGGC |
| | c.1340T > C (p.Leu447Pro) | | | CCTATCATGTGAAGGTACAT′] |
| 2505 | NM_000083.2(CLCN1): | 1180 | CLCN1 | [′CCCCGCGAAGCCATCAGTACTTTGTYTGACA |
| | c.1283T > C (p.Phe428Ser) | | | ACAATACATGGGTGAAACAC′] |
| 2506 | NM_003995.3(NPR2): c.226T > C | 4882 | NPR2 | [′CAGCTCCGAACTGGAAGGCGCCTGCYCTGA |
| | (p.Ser76Pro) | | | GTACCTGGCACCGCTGAGCGC′] |
| 2507 | NM_004700.3(KCNQ4): c.808T > | 9132 | KCNQ4 | [′GGACGCCAACTCCGACTTCTCCTCCYACGCC |
| | C (p.Tyr270His) | | | GACTCGCTCTGGTGGGGGAC′] |
| 2508 | NM_004700.3(KCNQ4): c.823T > | 9132 | KCNQ4 | [′CTTCTCCTCCTACGCCGACTCGCTCYGGTGG |
| | C (p.Trp275Arg) | | | GGGACGGTGCGTGAGGGTCT′] |
| 2509 | NM_005097.3(LGI1): c.124T > C | 9211 | LGI1 | [′GGGGAAGAAACCAGCGAAGCCAAAABGCCC |
| | (p.Cys42Arg) | | | TGCCGTGTGTACTTGTACCAA′] |
| 2510 | NM_005572.3(LMNA): | 4000 | LMNA | [′CAGCTCAGCCAGCTCCAGAAGCAGGYGATA |
| | c.936 + 2T > C | | | CCCCACCTCACCCCTCTCTCC′] |
| 2511 | NM_006009.3(TUBA1A): | 7846 | TUBA1A | [′AAACGTGCCTTTGTTCACTGGTACGYTGGGG |
| | c.1226T > C (p. Val409Ala) | | | AGGGGATGGAGGAAGGTGAG′] |
| 2512 | NM_001081550.1(THOC2): | 57187 | THOC2 | [′GACGTGTTCAATATGTTCTGTTACCYTGGTC |
| | c.1313T > C (p.Leu438Pro) | | | CTCACCTTTCTCACGATCCC′] |
| 2513 | NM_001081550.1(THOC2): | 57187 | THOC2 | [′ACATCAACAGAAAACTCCAAATTTTYCCAC |
| | c.3034T > C (p.Ser1012Pro) | | | ACTTCTTTGCTATGATCGAGT′] |
| 2514 | NM_001081550.1(THOC2): | 57187 | THOC2 | [′GATTATATAAAGCGAGTGCCTTCAAYTGATG |
| | c.2399T > C (p.Ile800Thr) | | | TACTCTGTAATGAATTTCAT′] |
| 2515 | NM_001356.4(DDX3X): | 1654 | DDX3X | [′CAGGTAGCAGCAAGAGGACTGGACAYTTCA |
| | c.1520T > C (p.Ile507Thr) | | | AATGTGAAACATGTTATCAAT′] |
| 2516 | NM_000158.3(GBE1): | 2632 | GBE1 | [′TGAATAAAAATCACAGTTATTACTTRCCAAG |
| | c.691 + 2T > C | | | GCCTTTGATTCTTGGTAGTA′] |
| 2517 | NM_000719.6(CACNA1C): | 775 | CACNA1C | [′ATGAACATCTTCGTGGGCTTCGTCAYCGTCA |
| | c.3497T > C (p.Ile1166Thr) | | | CCTTTCAGGAGCAGGGGGAG′] |
| 2518 | NM_001105.4(ACVR1): c.587T > | 90 | ACVR1 | [′TGTACATCAGGAAGTGGCTCTGGTCYTCCTT |
| | C (p.Leu196Pro) | | | TTCTGGTACAAAGAACAGTG′] |
| 2519 | NM_024531.4(SLC52A2): | 79581 | SLC52A2 | [′CCCTACGGGCGTCTGGCCTACCACCYGGCTG |
| | c.935T > C (p.Leu312Pro) | | | TGGTGCTGGGCAGTGCTGCC′] |
| 2520 | NM_033409.3(SLC52A3): | 113278 | SLC52A3 | [′AGCTGCCTTTCCGAAGTGCCCATCAYCTTCA |
| | c.224T > C (p.Ile75Thr) | | | CCCTGCTGGGCGTGGGAACC′] |
| 2521 | NM_033409.3(SLC52A3): | 113278 | SLC52A3 | [′GGTCTGCGTCTTCGGAATGGGCTCCYGGGTG |
| | c.49T > C (p.Trp17Arg) | | | ACCATCAATGGGCTCTGGGT′] |
| 2522 | NM_133433.3(NIPBL): | 25836 | NIPBL | [′CCATACCTTACCACTAAATGTAGTGYAAGTA |
| | c.6108 + 2T > C | | | TAGAGCTGTCTTATTCTTGT′] |
| 2523 | NM_002397.4(MEF2C): c.2T > C | 4208 | MEF2C | [′CGTAATCTGAATCTTTTTTCTCCCCDTAGTCC |
| | (p.Met1Thr) | | | CCGTTTTTCTTCTCTCTCT′] |
| 2524 | NM_000124.3(ERCC6): | 2074 | ERCC6 | [′AATACTCGCTGACCCTGCTTGTGCCRTATTT |
| | c.2551T> C (p.Trp851Arg) | | | TCAACAAAGACTCAACAACA′] |
| 2525 | NM_000208.2(INSR): | 3643 | INSR | [′GTTCATGCTGTTCTACAAAGAGGCGYAAGT |
| | c.1610 + 2T > C | | | AGAAGAGTTAGAGAGACGCTG′] |
| 2526 | NM_000071.2(CBS): c.325T > C | 875 | CBS | [′CTCCCGCCCGCGTTGAAGAACTCACRCTTGG |
| | (p.Cys109Arg) | | | CCACTGGGAGGCAGAGATGA′] |
| 2527 | NM_004550.4(NDUFS2): | 4720 | NDUFS2 | [′CATTATGCTCTCCACAGTGGAGTGAYGCTTC |
| | c.875T> C (p.Met292Thr) | | | GGGGCTCAGGCATCCAGTGG′] |
| 2528 | NM_003119.3(SPG7): c.2228T > | 6687 | SPG7 | [′AAGGAAGTGATAAACTATGAGGACAYTGAG |
| | C (p.Ile743Thr) | | | GCTCTCATTGGCCCGCCGCCC′] |
| 2529 | NM_000546.5(TP53): c.584T > C | 7157 | TP53 | [′CACACGCAAATTTCCTTCCACTCGGRTAAGA |
| | (p.Ile195Thr) | | | TGCTGAGGAGGGGCCAGACC′] |
| 2530 | NM_018972.2(GDAP1): c.347T > | 54332 | GDAP1 | [′AGGTTAATGCCTGATAAAGAAAGCAKGTAT |
| | C (p.Met116Thr) | | | TACCCACGGGTACAACATTAC′] |
| 2531 | NM_000070.2(CAPN3): c.566T > | 825 | CAPN3 | [′TGCCTGCCAACGTACAACAATCAACYGGTTT |
| | C (p.Leu189Pro) | | | TCACCAAGTCCAACCACCGC′] |
| 2532 | NM_000070.2(CAPN3): | 825 | CAPN3 | [′TTAATTCCTCCATTTTCCCACCAGAYGGAAG |
| | c.1117T > C (p.Trp373Arg) | | | GACTGGAGCTTTGTGGACAA′] |
| 2533 | NM_181534.3(KRT25): c.950T > | 147183 | KRT25 | [′AAATTGTTCTTTTCATACCGTGGCTRGGAGA |
| | C (p.Leu317Pro) | | | GACTGAAGTTCAATTTCCAG′] |
| 2534 | NM_001002294.2(FMO3): | 2328 | FMO3 | [′CCAGGTGTATTCAATGGAAAGCGTGBCCTG |
| | c.560T > C (p. Val187Ala) | | | GTGGTTGGCCTGGGGAATTCG′] |
| 2535 | NM_001080522.2(CC2D2A): | 57545 | CC2D2A | [′GAAATTCAAGCTGAAATAAGTGAACYGTTA |
| | c.1676T > C (p.Leu559Pro) | | | GAAGAGCACACGGAGGAGTAC′] |
| 2536 | NM_001080522.2(CC2D2A): | 57545 | CC2D2A | [′TACCAAGTCATATTTTCTTCACAGAYTGATG |
| | c.3596T > C (p.Ile1199Thr) | | | GAACATTTAAAATAGATATT′] |
| 2537 | NM_025114.3(CEP290): | 80184 | CEP290 | [′CCTGTAACAAATGTATTAAATATTCRTTCTG |
| | c.2343T > C (p.Asn781=) | | | AGAATTAATGATACTGGCAC′] |
| 2538 | NM_030578.3(B9D2): c.107T > C | 80776 | B9D2 | [′CGTTTGGCCCTCCCGCACGCCTGACRGGAGC |
| | (p.Leu36Pro) | | | TTCCATGCCGCCCCTGCAGT′] |
| 2539 | NM_004304.4(ALK): c.3520T > C | 238 | ALK | [′ACATCCCTCTCTGCTCTGCAGCAAAKTCAAC |
| | (p.Phe1174Leu) | | | CACCAGAACATTGTTCGCTG′] |

TABLE 7
Diseases/disorders containging A to G Changes. The table includes human gene
mutations that may be corrected by changing a guanine (G) to adenine (A). The gene name,
gene symbol,and Gene ID are indicated.

| SEQ | | | | |
|---|---|---|---|---|
| ID | | Gene | Gene | |
| NO: | Name | ID | Symbol | Flanks |
| 3144 | NM_017547.3(FOXRED1): | 55572 | FOXRED1 | [′GTGGGCCCCCACCCGCTAGTTGTCAVCATG |
| | c.1289A > G (p.Asn430Ser) | | | TACTTTGCTACTGGCTTCAGT′] |
| 3145 | NM_001006657.1(WDR35): | 57539 | WDR35 | [′CTAGTGCGGCTGTGGTTGCTTTTTCVGATT |
| | c.25 − 2A > G | | | TCCATTCCCAATAACGTGAAG′] |
| 3146 | NM_001006657.1(WDR35): | 57539 | WDR35 | [′TATGTTTTCAGAAACTTGGATCCTGRGGTA |
| | c.1877A > G (p.Glu626Gly) | | | AAAACAAGAAATGAGTGTTAA′] |
| 3147 | NM_000374.4(UROD): | 7389 | UROD | [′GGCAACCTGGACCCCTGTGCCTTGTRTGCA |
| | c.932A > G (p.Tyr311Cys) | | | TCTGAGGTAACAGCCAGGGCC′] |
| 3148 | NM_004315.4(ASAH1): | 427 | ASAH1 | [′ACAGAGGACTGCAGAAAATCAACCTRTCC |
| | c.155A > G (p.Tyr52Cys) | | | TCCTTCAGGACCAACGTGAGTA′] |
| 3149 | NM_004315.4(ASAH1): | 427 | ASAH1 | [′GGGTAGATGGTATGTGGTACAAACARATT |
| | c.1006A > G (p.Asn336Asp) | | | ATGACCGTTGGAAACATCCCTT′] |
| 3150 | NM_000071.2(CBS): c.1150A > G | 875 | CBS | [′GGTGACTCCCCCATCCCGCAGGACCRAGTT |
| | (p.Lys384Glu) | | | CCTGAGCGACAGGTGGATGCT′] |
| 3151 | NM_000274.3(OAT): c.734A > G | 4942 | OAT | [′GGCGTTGTTGTTCCGGATCCAGGTTRCCTA |
| | (p.Tyr245Cys) | | | ATGGGAGTGCGAGAGCTCTGC′] |
| 3152 | NM_001385.2(DPYS): | 1807 | DPYS | [′GATAACTGCACTTTCAACACCTGCCRGAA |
| | c.1001A > G (p.Gln334Arg) | | | AGCTCTTGGGAAGGATGATTTT′] |
| 3153 | NM_024598.3(USB1): c.502A > G | 79650 | USB1 | [′GATTTACACCAATCAAGAGAAAACCRGGT |
| | (p.Arg168Gly) | | | GGGTCCTCCCAACCCCCAATCA′] |
| 3154 | NM_001194958.2(KCNJ18): | 100134444 | KCNJ18 | [′AGTGCGAAGGATCTGGTAGAGAACARGTT |
| | c.1097A > G (p.Lys366Arg) | | | CCTGCTGCCCAGTGCCAACTCC′] |
| 3155 | NM_001914.3(CYB5A): | 1528 | CYB5A | [′TTTTTATTCTACCTCTCCTTTGAACRGCATC |
| | c.130 − 2A > G | | | CTGGTGGGGAAGAAGTTTTA′] |
| 3156 | NM_000398.6(CYB5R3): | 1727 | CYB5R3 | [′CGCTTCAAGCTCTGGTACACGCTGGRCAG |
| | c.719A > G (p.Asp240Gly) | | | AGCCCCTGAAGGTGAGTGAGGG′] |
| 3157 | NM_004628.4(XPC): | 7508 | XPC | [′ATTATTATTGTTATTACTATTACTGRTTTTT |
| | c.413 − 24A > G | | | AAAAATGCTTGTTGATAGAA′] |
| 3158 | NM_001134363.2(RBM20): | 282996 | RBM20 | [′TGGCCCAGAAAGGCCGCGGTCTCGTRGTC |
| | c.1909A > G (p.Ser637Gly) | | | CGGTGAGCCGGTCACTCTCCCC′] |
| 3159 | NM_000552.3(VWF): c.2384A > G | 7450 | VWF | [′GAGTGTACCAAAACGTGCCAGAACTRTGA |
| | (p.Tyr795Cys) | | | CCTGGAGTGCATGAGCATGGGC′] |
| 3160 | NM_000552.3(VWF): c.1583A > G | 7450 | VWF | [′ACCTGCGGCCTGTGTGGGAATTACARTGG |
| | (p.Asn528Ser) | | | CAACCAGGGCGACGACTTCCTT′] |
| 3161 | NM_144573.3(NEXN): c.1955A > G | 91624 | NEXN | [′ACTTTCCCAGAAGATGGAGGAGAGTRTAT |
| | (p.Tyr652Cys) | | | GTGTAAAGCAGTCAACAATAAA′] |
| 3162 | NM_000267.3(NF1): c.4267A > G | 4763 | NF1 | [′CGAAAGGGGCTTGAAGTTAATGTCARAGG |
| | (p.Lys1423Glu) | | | TGAATTATTTTGATAATCTAGC′] |
| 3163 | NM_000267.3(NF1): | 4763 | NF1 | [′TCTCTTTGTCTTTCTCTTTTTTAAARAATTC |
| | c.1642 − 8A > G | | | AGGCTCTGCTGGTTCTTCAT′] |
| 3164 | NM_000267.3(NF1): c.1466A > G | 4763 | NF1 | [′CCTACAGACCTGGAGACAAGAAGCTRTAA |
| | (p.Tyr489Cys) | | | GTATCTTCTCTTGTCCATGGTG′] |
| 3165 | NM_000267.3(NF1): | 4763 | NF1 | [′AAAAACATGTTATTTTCCTTCTTCARCTAG |
| | c.5944 − 5A > G | | | ATTACAGATCTGCTTGATGTT′] |
| 3166 | NM_000308.2(CTSA): | 5476 | CTSA | [′TACCATGGCCTTCTGGGGAACAGGTRTGG |
| | c.746 + 3A > G | | | GATAGGGCAGTTGGGCAATCTC′] |
| 3167 | NM_000308.2(CTSA): c.200A > G | 5476 | CTSA | [′CTGGCCAAGCAGCCGTCTTTCCGCCRGTAC |
| | (p.Gln67Arg) | | | TCCGGCTACCTCAAAGGCTCC′] |
| 3168 | NM_000308.2(CTSA): c.1238A > G | 5476 | CTSA | [′CTTTAGAAATACCAGATCCTATTATRTAAT |
| | (p.Tyr413Cys) | | | GGAGATGTAGACATGGCCTGC′] |
| 3169 | NM_000308.2(CTSA): c.1411A > G | 5476 | CTSA | [′CTCCCACATCGCCTTTCTCACGATCRAGGT |
| | (p.Lys471Glu) | | | AGGGACTGGGCCTGCTGAGAG′] |
| 3170 | NM_000353.2(TAT): | 6898 | TAT | [′TTCCAAACACAGTAGGGTCCCCTTTYTATG |
| | c.236 − 5A > G | | | GGAGGAAAACACAAAAGGAGC′] |
| 3171 | NM_000097.5(CPOX): c.1210A > G | 1371 | CPOX | [′TAATCTGCTGTATGATCGGGGCACARAGTT |
| | (p.Lys404Glu) | | | TGGCCTCTTCACTCCAGGATC′] |
| 3172 | NM_000317.2(PTS): c.155A > G | 5805 | PTS | [′AACAATCCAAATGGCCATGGGCACARTTA |
| | (p.Asn52Ser) | | | TAAAGGTGAGAGAAAAACTGAT′] |
| 3173 | NM_000317.2(PTS): c.139A > G | 5805 | PTS | [′ACTGTTTGGGAAATGCAACAATCCARATG |
| | (p.Asn47Asp) | | | GCCATGGGCACAATTATAAAGG′] |
| 3174 | NM_000317.2(PTS): c.347A > G | 5805 | PTS | [′GAAAATGTAGCTGTTTATATCTGGGRCAAC |
| | (p.Asp116Gly) | | | CTCCAGAAAGTTCTTCCTGTA′] |
| 3175 | NM_000320.2(QDPR): c.449A > G | 5860 | QDPR | [′CCCCATTTCCCAGGTATGATCGGGTRCGGC |
| | (p.Tyr150Cys) | | | ATGGCCAAGGGTGCTGTTCAC′] |
| 3176 | NM_173560.3(RFX6): | 222546 | RFX6 | [′TCTATTTTTCTTTATCATCCCTTCARCTGGC |
| | c.224 − 12A > G | | | AATCAGAAATGCACTTAAAC′] |
| 3177 | NM_001999.3(FBN2): | 2201 | FBN2 | [′TTCATCAATATCTGTGAAAACAGCAYTGC |
| | c.3725 − 15A > G | | | AACCACATTGTCAGGTCTGCTT′] |
| 3178 | NM_001040613.2(TMEM70): | 54968 | TMEM70 | [′GATCCTCTCTCTTTTTTTCCCATTTRGGTGT |
| | c.*7 − 2A > G | | | GAAATGTTTCTCTTATTCTA′] |
| 3179 | NM_152416.3(NDUFAF6): | 137682 | NDUFAF6 | [′AGGGCCTTTAATGTGGAACTGGCTCRGGCT |
| | c.296A > G (p.Gln99Arg) | | | GGTATTAAGATACCTTAAAAT′] |
| 3180 | NM_000140.3(FECH): | 2235 | FECH | [′ACTAAAACGATTGTAACACTGTAGAYACC |
| | c.1137 + 3A > G | | | TTAGAGAACAATGGATTTCCAT′] |
| 3181 | NM_000277.1(PAH): c.1A > G | 5053 | PAH | [′GAGACCTCACTCCCGGGGAGCCAGCDTGT |
| | (p.Met1Val) | | | CCACTGCGGTCCTGGAAAACCC′] |
| 3182 | NM_000277.1(PAH): c.611A > G | 5053 | PAH | [′TCCTTGTATAAAACCCATGCTTGCTRTGAG |
| | (p.Tyr204Cys) | | | TACAATCACATTTTTCCACTT′] |
| 3183 | NM_000277.1(PAH): c.1241A > G | 5053 | PAH | [′ATACCTCGGCCCTTCTCAGTTCGCTRCGAC |
| | (p.Tyr414Cys) | | | CCATACACCCAAAGGATTGAG′] |
| 3184 | NM_000277.1(PAH): c.662A > G | 5053 | PAH | [′CTTGAAAAGTACTGTGGCTTCCATGRAGAT |
| | (p.Glu221Gly) | | | AACATTCCCCAGCTGGAAGAC′] |
| 3185 | NM_000277.1(PAH): c.916A > G | 5053 | PAH | [′TTCTATTTTCCCCCAATTACAGGAARTTGG |
| | (p.Ile306Val) | | | CCTTGCCTCTCTGGGTGCACC′] |
| 3186 | NM_000277.1(PAH): c.1169A > G | 5053 | PAH | [′TTCCAGCCCCTGTATTACGTGGCAGRGAGT |
| | (p.Glu390Gly) | | | TTTAATGATGCCAAGGAGAAA′] |
| 3187 | NM_000277.1(PAH): | 5053 | PAH | [′TGTCATCCTTTGGTGAATTACAGGTVTGAC |
| | c.1065 + 3A > G | | | CTTCACAGGAACCAAGGATAG′] |
| 3188 | NM_000277.1(PAH): c.227A > G | 5053 | PAH | [′AGACCTTCTCGTTTAAAGAAAGATGVGTA |
| | (p.Glu76Gly) | | | TGAATTTTTCACCCATTTGGAT′] |
| 3189 | NM_000130.4(F5): c.1000A > G | 2153 | F5 | [′CATTAAAAACTGCCCAAAGAAAACCRGGA |
| | (p.Arg334Gly) | | | ATCTTAAGAAAATAACTCGTGA′] |
| 3190 | NM_000130.4(F5): c.5189A > G | 2153 | F5 | [′GGCTCTGCCTGTCGGGCTTGGGCCTRCTAC |
| | (p.Tyr1730Cys) | | | TCAGCTGTGAACCCAGTAGGT′] |
| 3191 | NM_000512.4(GALNS): | 2588 | GALNS | [′TTGGTCCCCGCGCAGCCCCAGCTCARCGTG |
| | c.1460A > G (p.Asn487Ser) | | | TGCAACTGGGCGGTCATGGTA′] |
| 3192 | NM_015702.2(MMADHC): | 27249 | MMADHC | [′ACTCTTTTTGAAACTGATGAACGCTRCCGA |
| | c.746A > G (p.Tyr249Cys) | | | CATTTAGGATTCTCTGTTGAT′] |
| 3193 | NM_015141.3(GPD1L): c.370A > G | 23171 | GPD1L | [′TAACTTCTTGGCATCCTTGTAGGGCRTAGA |
| | (p.Ile124Val) | | | CGAGGGCCCCGAGGGGCTGAA′] |
| 3194 | NM_013319.2(UBIAD1): | 29914 | UBIAD1 | [′GTGCACGGGGCCGGTAATTTGGTCARCAC |
| | c.305A > G (p.Asn102Ser) | | | TTACTATGACTTTTCCAAGGGC′] |
| 3195 | NM_013319.2(UBIAD1): | 29914 | UBIAD1 | [′CATTGACCACAAAAAGAGTGATGACRGGA |
| | c.355A > G (p.Arg119Gly) | | | CACTTGTGGACCGAATCTTGGA′] |
| 3196 | NM_013319.2(UBIAD1): | 29914 | UBIAD1 | [′AGCACCGAGGCCATTCTCCATTCCARCAAC |
| | c.695A > G (p.Asn232Ser) | | | ACCAGGGACATGGAGTCCGAC′] |
| 3197 | NM_013319.2(UBIAD1): | 29914 | UBIAD1 | [′TACTATGACTTTTCCAAGGGCATTGRCCAC |
| | c.335A > G (p.Asp112Gly) | | | AAAAAGAGTGATGACAGGACA′] |
| 3198 | NM_000046.3(ARSB): c.629A > G | 411 | ARSB | [′GTTGCAACAGGATATAAAAATATGTRTTC |
| | (p.Tyr210Cys) | | | AACAAACATATTCACCAAAAGG′] |
| 3199 | NM_006331.7(EMG1): c.257A > G | 10436 | EMG1 | [′CGGGACCCTGGGGAAGCGCGGCCAGRTAT |
| | (p.Asp86Gly) | | | CACCCACCAGGTAACTCCAGGG′] |
| 3200 | NM_000181.3(GUSB): c.1484A > G | 2990 | GUSB | [′CTGCTGCTCTGGTCCTAGGCTCCGTRTGTG |
| | (p.Tyr495Cys) | | | GATGTGATCTGTTTGAACAGC′] |
| 3201 | NM_000067.2(CA2): c.754A > G | 760 | CA2 | [′CTGGCGCCCAGCTCAGCCACTGAAGRACA |
| | (p.Asn252Asp) | | | GGCAAATCAAAGCTTCCTTCAA′] |
| 3202 | NM_000067.2(CA2): c.52A > G | 760 | CA2 | [′TCCCCCAGGACCTGAGCACTGGCATVAGG |
| | (p.Lys18Glu) | | | ACTTCCCCATTGCCAAGGGAGA′] |
| 3203 | NM_000404.2(GLB1): c.947A > G | 2720 | GLB1 | [′TTTATAGGTGGGACCAATTTTGCCTRTTGG |
| | (p.Tyr316Cys) | | | AATGGTAAGAGCACTTTAATA′] |
| 3204 | NM_000404.2(GLB1): c.1498A > G | 2720 | GLB1 | [′TTTGCAGGGTTTGGTTTCTAACCTGRCTCT |
| | (p.Thr500Ala) | | | CAGTTCCAATATCCTCACGGA′] |
| 3205 | NM_000404.2(GLB1): | 2720 | GLB1 | [′GAGAGTCAGGTTAGAAACCAAACCCYGCA |
| | c.1480 − 2A > G | | | AAGCAGAAACAGAGCACAGTGA′] |
| 3206 | NM_000404.2(GLB1): c.1772A > G | 2720 | GLB1 | [′ATTAATGGCTTTAACCTTGGCCGCTRTTGG |
| | (p.Tyr591Cys) | | | CCAGCCCGGGGCCCTCAGTTG′] |
| 3207 | NM_203447.3(DOCK8): | 81704 | DOCK8 | [′ACTCTGAGCGTTAGCAGCTTTTTCARGCAG |
| | c.1418A > G (p.Lys473Arg) | | | GTATCTCTTCACATTACAGTG′] |
| 3208 | NM_000275.2(OCA2): c.1465A > G | 4948 | OCA2 | [′TGCCACTGCCATCGGGGACCCTCCARATGT |
| | (p.Asn489Asp) | | | CATTATTGTTTCCAACCAAGA′] |
| 3209 | NM_152778.2(MFSD8): c.362A > G | 256471 | MFSD8 | [′ATTTCCGTGGCAGCCAACTGCCTCTVTGCA |
| | (p.Tyr121Cys) | | | TATCTCCACATCCCAGCTTCT′] |
| 3210 | NM_139241.3(FGD4): | 121512 | FGD4 | [′ACTGTTCATTTTTCTTTTAAATTTARGGTGG |
| | c.1762 − 2A > G | | | TTTGTTGGAAATGCTCCGAC′] |
| 3211 | NM_020223.3(FAM20C): | 56975 | FAM20C | [′TGTGACACTTTCTGCCTCTCTCCGCRGGAA |
| | c.1364 − 2A > G | | | ACATGGACCGTCACCACTACG′] |
| 3212 | NM_138387.3(G6PC3): c.346A > G | 92579 | G6PC3 | [′GGCAGGCAGCCCTTCTGGACACTGCRTGA |
| | (p.Met116Val) | | | TCACAGGAGCAGCCCTCTGGCC′] |
| 3213 | NM_018122.4(DARS2): c.133A > G | 55157 | DARS2 | [′TTTTTTTTTTTTTTTTAAAGAATTCRGTAGC |
| | (p.Ser45Gly) | | | TTTGTTGTCCGGACCAACAC′] |
| 3214 | NM_000642.2(AGL): | 178 | AGL | [′CATCTTTTATTTAACTTAAATTTCARTCATT |
| | c.4260 − 12A > G | | | TTGCAGTGATATGGTTTACT′] |
| 3215 | NM_000642.2(AGL): c.3439A > G | 178 | AGL | [′TCTACTGGGTGAAGGAATTTATGCCRGATA |
| | (p.Arg1147Gly) | | | CAATTGTCGGGATGCTGTGTG′] |
| 3216 | NM_145014.2(HYLS1): c.632A > G | −1 | — | [′TTAAGCCGAAACCGGGGCAAGACAGRCCG |
| | (p.Asp211Gly) | | | GGTAGCCCGGTATTTTGAGTAC′] |
| 3217 | NM_014362.3(HIBCH): c.365A > G | 26275 | HIBCH | [′GCTCCAGTTTTCTTCAGAGAAGAATRTATG |
| | (p.Tyr122Cys) | | | CTGAATAATGCTGTTGGTATG′] |
| 3218 | NM_014846.3(KIAA0196): | 9897 | KIAA0196 | [′TTTTCTTATTTCTGTATTACTAGAARACCTT |
| | c.1411A > G (p.Asn471Asp) | | | CAAGCTTGGTTCAGAGAGAT′] |
| 3219 | NM_000505.3(F12): c.158A > G | 2161 | F12 | [′GAGCCCTGCCACTTCCCCTTCCAGTRCCAC |
| | (p.Tyr53Cys) | | | CGGCAGCTGTACCACAAATGT′] |
| 3220 | NM_001029871.3(RSPO4): | 343637 | RSPO4 | [′TTCATCCGCCGGGAAGGCATCCGCCRGTA |
| | c.194A > G (p.Gln65Arg) | | | CGGCAAGTGCCTGCACGACTGT′] |
| 3221 | NM_017654.3(SAMD9): | 54809 | SAMD9 | [′TAAAAGACTGGAAAGACTTGTTCACRAAG |
| | c.4483A > G (p.Lys1495Glu) | | | GAAAAATTGACCAGTGCTTTAA′] |
| 3222 | NM_152419.2(HGSNAT): | 138050 | HGSNAT | [′TGTCTTAATTTTACCTAATGTTTGTRGGTTG |
| | c.372 − 2A > G | | | GAATACAGATTTGGAGAATT′] |
| 3223 | NM_001174089.1(SLC4A11): | 83959 | SLC4A11 | [′CATGAGCTCCCTGCCCTACATGAAGRTGAT |
| | c.2518A > G (p.Met840Val) | | | CTTTCCCCTCATCATGATCGC′] |
| 3224 | NM_012073.4(CCT5): c.440A > G | 22948 | CCT5 | [′CAGGCTGCTCGTGTTGCTATTGAACRCCTG |
| | (p.His147Arg) | | | GACAAGATCAGCGATAGCGTC′] |
| 3225 | NM_025114.3(CEP290): | 80184 | CEP290 | [′CACCTGGCCCCAGTTGTAATTGTGARTATC |
| | c.2991 + 1655A > G | | | TCATACCTATCCCTATTGGCA′] |
| 3226 | NM_153704.5(TMEM67): | 91147 | TMEM67 | [′TATTAAAACAGTTGTAACTGTTTATRGGAG |
| | c.870 − 2A > G | | | ACAGAATCTTCCTTGGCTGTT′] |
| 3227 | NM_153704.5(TMEM67): | 91147 | TMEM67 | [′TATCAGGTTTCTTTCTCAGTCACATDTGAA |
| | c.1538A > G (p.Tyr513Cys) | | | ATGGATCATGGAGAAGCACAT′] |
| 3228 | NM_001041.3(SI): c.350A > G | 6476 | SI | [′GTTGATAATCATGGTTATAACGTTCRAGAC |
| | (p.Gln117Arg) | | | ATGACAACAACAAGTATTGGT′] |
| 3229 | NM_015697.7(COQ2): c.890A > G | 27235 | COQ2 | [′TCTGGAGTTATGTGGACACTAATATRTGAT |
| | (p.Tyr297Cys) | | | ACTATTTATGCCCATCAGGTA′] |
| 3230 | NM_015697.7(COQ2): c.683A > G | 27235 | COQ2 | [′GCACTGGGTGTTCTTCTGTGTCTAARTTAC |
| | (p.Asn228Ser) | | | TACAGGTATATTAAACGTTTT′] |
| 3231 | NM_000190.3(HMBS): c.1A > G | 3145 | HMBS | [′CACAGCCTACTTTCCAAGCGGAGCCRTGTC |
| | (p.Met1Val) | | | TGGTAACGGCAATGCGGCTGC′] |
| 3232 | NM_004035.6(ACOX1): c.832A > G | 51 | ACOX1 | [′GAGTAACAAGCTGACTTACGGGACCRTGG |
| | (p.Met278Val) | | | TGTTTGTCAGGTCCTTCCTTGT′] |
| 3233 | NM_004035.6(ACOX1): c.926A > G | 51 | ACOX1 | [′ATCCGATACAGCGCTGTGAGGCACCRGTC |
| | (p.Gln309Arg) | | | TGAAATCAAGCCAGGGTAAGGA′] |
| 3234 | NM_000237.2(LPL): c.548A > G | 4023 | LPL | [′TGCTTTTTTCCCTTTTAAGGCCTCGRTCCAG |
| | (p.Asp183Gly) | | | CTGGACCTAACTTTGAGTAT′] |
| 3235 | NM_000237.2(LPL): c.953A > G | 4023 | LPL | [′TGCAACAATCTGGGCTATGAGATCARTAA |
| | (p.Asn318Ser) | | | AGTCAGAGCCAAAAGAAGCAGC′] |
| 3236 | NM_001171993.1(HPD): | 3242 | HPD | [′TACATCGGCCAATTCTTGCCTGGATRTGAG |
| | c.362A > G (p.Tyr121Cys) | | | GCCCCAGCGTTCATGGACCCC′] |
| 3237 | NM_139281.2(WDR36): | 134430 | WDR36 | [′CTTCTTGTCACAAATGGCGCTGACARTGCT |
| | c.1064A > G (p.Asn355Ser) | | | CTTAGGGTATTATGATTATTG′] |
| 3238 | NM_001127255.1(NLRP7): | −1 | —— | [′CTCACAAACCTGGACTTGAGTATCARCCA |
| | Ac.2738 > G (p.Asn913Ser) | | | GATAGCTCGTGGATTGTGGATT′] |
| 3239 | NM_016417.2(GLRX5): c.294A > G | 51218 | GLRX5 | [′TGCTGGACGACCCGGAGCTCCGACARGGT |
| | (p.Gln98=) | | | CAGGCCAGTGTGCCGGGCAGGC′] |
| 3240 | NM_024884.2(L2HGDH): c.293A > | 79944 | L2HGDH | [′ACTGGACATAACAGTGGTGTCATACRTAG |
| | G (p.His98Arg) | | | TGGAATTTATTATAAACCTGAG′] |
| 3241 | NM_000382.2(ALDH3A2): | 224 | ALDH3A2 | [′ACATCCAGTGGAGGTGTCACAGGCARTGA |
| | c.1157A > G (p.Asn386Ser) | | | CGTCATTATGCACTTCACGCTC′] |
| 3242 | NM_018105.2(THAP1): c.266A > G | 55145 | THAP1 | [′TTTCTTTGTACTGAGCCACATGACARGGTA |
| | (p.Lys89Arg) | | | ATATGCATTTTAAAATATTGG′] |
| 3243 | NM_000785.3(CYP27B1): | 1594 | CYP27B1 | [′GCCCTGGTTCGGGACGTGGCGGGGGRATT |
| | c.566A > G (p.Glu189Gly) | | | TTACAAGTTCGGACTGGAAGGT′] |
| 3244 | NM_182548.3(LHFPL5): | 222662 | LHFPL5 | [′TTCATCTGCAACACGGCCACAGTCTRTAAG |
| | c.380A > G(p.Tyr127Cys) | | | ATCTGTGCATGGATGCAGCTG′] |
| 3245 | NM_015040.3(PIKFYVE): | 200576 | PIKFYVE | [′TTCAAAGAAATGGAGAACAGGAGGARGA |
| | c.3308A > G (p.Lys1103Arg) | | | AACAGCTGCTCAGGGATCTCTCT′] |
| 3246 | NM_138701.3(MPLKIP): | 136647 | MPLKIP | [′GTTGGAAAATTATTTCAAGCCTTCARTGCT |
| | c.430A > G (p.Met144Val) | | | TGAAGATCCTTGGGCTGGCCT′] |
| 3247 | NM_152783.4(D2HGDH): | 728294 | D2HGDH | [′TGCCCTTGTCCCTCCAGGAGATGGTRACCT |
| | c.1315A > G (p.Asn439Asp) | | | GCACCTCAATGTGACGGCGGA′] |
| 3248 | NM_022912.2(REEP1): | 65055 | REEP1 | [′AACTTGTGTTTTCATCTCTCTCTCCRGGTTT |
| | c.183 − 2A > G | | | CCATTCTATTATGAACTAAA′] |
| 3249 | NM_000060.3(BTD): c.755A > G | 686 | BTD | [′ACATGCTTTGATATATTGTTCTTTGRCCCT |
| | (p.Asp252Gly) | | | GCCATCAGAGTCCTCAGAGAC′] |
| 3250 | NM_022132.4(MCCC2): | 64087 | MCCC2 | [′TGTGGCCTGTGCCCAAGTGCCTAAGDTAA |
| | c.1309A > G (p.Ile437Val) | | | CCCTCATCATTGGGGGCTCCTA′] |
| 3251 | NM_022132.4(MCCC2): c.569A > G | 64087 | MCCC2 | [′GCAGATGTGTTTCCAGATCGAGACCRCTTT |
| | (p.His190Arg) | | | GGCCGTACATTCTATAATCAG′] |
| 3252 | NM_198578.3(LRRK2): | 120892 | LRRK2 | [′ATCATCCGACTATATGAAATGCCTTRTTTT |
| | c.5096A > G (p.Tyr1699Cys) | | | CCAATGGGATTTTGGTCAAGA′] |
| 3253 | NM_198578.3(LRRK2): | 120892 | LRRK2 | [′TGTGACTAGAAATAAAATATCAGGGRTAT |
| | c.3364A > G(p.Ile1122Val) | | | GCTCCCCCTTGAGACTGAAGGA′] |
| 3254 | NM_000022.2(ADA): | 100 | ADA | [′TTCCCAACCCCTTTCTTCCCTTCCCRGGGG |
| | c.219 − 2A > G | | | CTGCCGGGAGGCTATCAAAAG′] |
| 3255 | NM_000022.2(ADA): c.290A > G | 100 | ADA | [′ATGAAGGCCAAAGAGGGCGTGGTGTDTGT |
| | (p.Tyr97Cys) | | | GGAGGTGCGGTACAGTCCGCAC′] |
| 3256 | NM_017780.3(CHD7): c.3082A > G | 55636 | CHD7 | [′TTTAGTAATTGCCCCATTGTCCACARTCCC |
| | (p.Ile1028Val) | | | CAACTGGGAAAGGGAATTCCG′] |
| 3257 | NM_017780.3(CHD7): c.164A > G | 55636 | CHD7 | [′TTTGCCTCTTTACAGCCATCCCTTCRTCATC |
| | (p.His55Arg) | | | CTTCAACTAATCAAAATCAA′] |
| 3258 | NM_152443.2(RDH12): c.677A > G | 145226 | RDH12 | [′GTCCCAGGCACCGGGGTCACCACCTRCGC |
| | (p.Tyr226Cys) | | | AGTGCACCCAGGCGTCGTCCGC′] |
| 3259 | NM_015335.4(MED13L): | 23389 | MED13L | [′TACCCGATGGTGCTAAAAAAGAAAGRAGA |
| | c.752A > G (p.Glu251Gly) | | | ATCGAAAGAGGAAGACGAGTTG′] |
| 3260 | NM_015335.4(MED13L): | 23389 | MED13L | [′GCTTTTTTTCCCCCCTTCCCTCTAGRTGATA |
| | c.6068A > G (p.Asp2023Gly) | | | TGTTTGTTGACCTTCCATTC′] |
| 3261 | NM_025265.3(TSEN2): c.926A > G | 80746 | TSEN2 | [′TATTTTCAGGCCTTTTTCTTGGTCTRTGCTC |
| | (p.Tyr309Cys) | | | TGGGATGTTTAAGTATTTAC′] |
| 3262 | NM_015384.4(NIPBL): | 25836 | NIPBL | [′AAAACAGACGTGACTATGCTCTTGTRTATA |
| | c.7289A > G (p.Tyr2430Cys) | | | GCAGACAATCTAGCCTGTTTT′] |
| 3263 | NM_207352.3(CYP4V2): | 285440 | CYP4V2 | [′GTCATCTTATCTACTTGCTTTCATCRGGGA |
| | c.1091 − 2A > G | | | AGTCTGACCGTCCCGCTACAG′] |
| 3264 | NM_024006.5(VKORC1): | 79001 | VKORC1 | [′CAGCTGTTCGCGCGTCTTCTCCTCCRGGTG |
| | c.172A >G (p.Arg58Gly) | | | TGCACGGGAGTGGGAGGCGTG′] |
| 3265 | NM_000551.3(VHL): c.491A > G | 7428 | VHL | [′TATACTCTGAAAGAGCGATGCCTCCRGGTT |
| | (p.Gln164Arg) | | | GTCCGGAGCCTAGTCAAGCCT′] |
| 3266 | NM_000101.3(CYBA): c.281A > G | 1535 | CYBA | [′AATTACTATGTTCGGGCCGTCCTGCRTCTC |
| | (p.His94Arg) | | | CTGTGAGTCCCCGTCCCGCAC′] |
| 3267 | NM_014874.3(MFN2): c.827A > G | 9927 | MFN2 | [′TTGTTTGGGCTCCAGGTGCGGCGGCRGCAC |
| | (p.Gln276Arg) | | | ATGGAGCGTTGTACCAGCTTC′] |
| 3268 | NM_015046.5(SETX): c.1807A > G | 23064 | SETX | [′GCAGAAGTCAGATCCACAAAAGTGTYACA |
| | (p.Asn603Asp) | | | TGGAGGTGCTTTGAATTTTATG′] |
| 3269 | NM_005609.2(PYGM): c.1A > G | 5837 | PYGM | [′CAGTCCGGCCCGCCCTCCTGCAGCCVTGTC |
| | (p.Met1Val) | | | CCGGCCCCTGTCAGACCAAGA′] |
| 3270 | NM_033071.3(SYNE1): | 23345 | SYNE1 | [′ACTTCACTTCTGTTTTATCCCTGTARTGTTT |
| | c.15705 − 12A > G | | | CTGAAGCATGCAGGAGAAAG′] |
| 3271 | NM_130468.3(CHST14): | 113189 | CHST14 | [′CTGTGCCAGCCTTGTGCCGTGCACTRTGAC |
| | c.878A > G (p.Tyr293Cys) | | | TTTGTGGGCTCCTATGAGAGG′] |
| 3272 | NM_206933.2(USH2A): | 7399 | USH2A | [′AAAAGTTTTGTATTACGAATTATACRGAAG |
| | c.14020A > G (p.Arg4674Gly) | | | ACAAATAGCAACTCAGCCTAG′] |
| 3273 | NM_000048.3(ASL): c.857A > G | 435 | ASL | [′AGCACGGGAAGCAGCCTGATGCCCCRGAA |
| | (p.Gln286Arg) | | | GAAAAACCCCGACAGTTTGGAG′] |
| 3274 | NM_001875.4(CPS1): c.1010A > G | 1373 | CPS1 | [′CAGGCTTTCATTACTGCTCAGAATCRTGGC |
| | (p.His337Arg) | | | TATGCCTTGGACAACACCCTC′] |
| 3275 | NM_001173464.1(KIF21A): | 55605 | KIF21A | [′GACCATTTCCAACATGGAGGCAGATRTGA |
| | c.2839A > G (p.Met947Val) | | | ATAGACTCCTCAAGGTGTGGAA′] |
| 3276 | NM_000026.2(ADSL): c.736A > G | 158 | ADSL | [′CATCACAGGGCAGACATATACACGARAAG |
| | (p.Lys246Glu) | | | TGGATATTGAAGTACTGTCTGT′] |
| 3277 | NM_024577.3(SH3TC2): | 79628 | SH3TC2 | [′AGCCATTGCTTTTCTCATTATCCACRGGCC |
| | c.530 − 2A > G | | | ACTTCTTCTGCAGAGCCCTGT′] |
| 3278 | NM_018249.5(CDK5RAP2): | 55755 | CDK5RAP | [′AGCATGGGAGATGATTTATCTTTCARTCTC |
| | c.4005 − 15A > G | | 2 | AATGGTTAAGGATTGAGGAAG′] |
| 3279 | NM_000346.3(SOX9): c.517A > G | 6662 | SOX9 | [′GCACAAGAAGGACCACCCGGATTACRAGT |
| | (p.Lys173Glu) | | | ACCAGCCGCGGCGGAGGAAGTC′] |
| 3280 | NM_144596.3(TTC8): | 123016 | TTC8 | [′GTTTACTGCCTTCTTAATGCTTTCCRGGAA |
| | c.115 − 2A > G | | | CCAGATCCTGAATTGCCAGTG′] |
| 3281 | NM_000243.2(MEFV): c.2080A > G | 4210 | MEFV | [′GAATGGCTACTGGGTGGTGATAATGDTGA |
| | (p.Met694Val) | | | AGGAAAATGAGTACCAGGCGTC′] |
| 3282 | NM_000243.2(MEFV): c.2084A > G | 4210 | MEFV | [′GGCTACTGGGTGGTGATAATGATGADGGA |
| | (p.Lys695Arg) | | | AAATGAGTACCAGGCGTCCAGC′] |
| 3283 | NM_006493.2(CLN5): c.1121A > G | 1203 | CLN5 | [′GTGCACAAACAGTTCTATTTGTTTTRTAAT |
| | (p.Tyr374Cys) | | | TTTGAATATTGGTTTTTACCT′] |
| 3284 | NM_000483.4(APOC2): c.1A > G | −1 | — | [′TCAATGTTCCAGGTCTCTGGACACTRTGGG |
| | (p.Met1Val) | | | CACACGACTCCTCCCAGCTCT′] |
| 3285 | NM_058172.5(ANTXR2): | 118429 | ANTXR2 | [′TGGCCAACTGTGGATGCTTCCTATTDTGGT |
| | c.1142A > G (p.Tyr381Cys) | | | GGTCGAGGGGTTGGAGGAATT′] |
| 3286 | NM_001128085.1(ASPA): | −1 | — | [′TATAAAATTATAGAGAAAGTTGATTRCCCC |
| | c.692A > G (p.Tyr231Cys) | | | CGGGATGAAAATGGAGAAATT′] |
| 3287 | NM_001128085.1(ASPA): | −1 | — | [′ATCTTTGGAGGAACCCATGGGAATGRGCT |
| | c.71A > G (p.Glu24Gly) | | | AACCGGAGTATTTCTGGTTAAG′] |
| 3288 | NM_014365.2(HSPB8): c.421A > G | 26353 | HSPB8 | [′TGGCATTGTTTCTAAGAACTTCACARAGAA |
| | (p.Lys141Glu) | | | AATCCAGTAAGTAACCTGGAG′] |
| 3289 | NM_000391.3(TPP1): c.857A > G | 1200 | TPP1 | [′CAGTACCTGATGAGTGCTGGTGCCARCATC |
| | (p.Asn286Ser) | | | TCCACCTGGGTCTACAGTAGC′] |
| 3290 | NM_000391.3(TPP1): | 1200 | TPP1 | [′TGTCCCTCATGCCGGCCTGGATTTTYTTTTT |
| | c.887 − 10A > G | | | TTTTTTTTTTGAGGGATGGG′] |
| 3291 | NM_182760.3(SUMF1): c.1A > G | 285362 | SUMF1 | [′CGGGTCACATGGCCCGCGGGACAACRTGG |
| | (p.Met1Val) | | | CTGCGCCCGCACTAGGGCTGGT′] |
| 3292 | NM_031443.3(CCM2): c.1A > G | 83605 | CCM2 | [′GGCCGCGGGAGCCGCACGCGGCGATRTGG |
| | (p.Met1Val) | | | AAGAGGAGGGCAAGAAGGGCAA′] |
| 3293 | NM_004183.3(BEST1): c.707A > G | 7439 | BEST1 | [′GACTGGATTAGTATCCCACTGGTGTRTACA |
| | (p.Tyr236Cys) | | | CAGGTGAGGACTAGGCTGGTG′] |
| 3294 | NM_004183.3(BEST1): c.680A > G | 7439 | BEST1 | [′ACTCAGTGTGGACACCTGTATGCCTRCGAC |
| | (p.Tyr227Cys) | | | TGGATTAGTATCCCACTGGTG′] |
| 3295 | NM_000158.3(GBE1): c.1634A > G | 2632 | GBE1 | [′TGTAATGCAGGTAATGAATTTGGGCRTCCT |
| | (p.His545Arg) | | | GAATGGTTAGACTTCCCAAGA′] |
| 3296 | NM_000158.3(GBE1): c.1883A > G | 2632 | GBE1 | [′GGTCTTCTTTTCATTTTCAACTTCCRTCCAA |
| | (p.His628Arg) | | | GCAAGAGCTACACTGACTAC′] |
| 3297 | NM_017890.4(VPS13B): | 157680 | VPS13B | [′CTTCTGCCCTGGGCCCTGCTTATCARTGAA |
| | c.8978A > G (p.Asn2993Ser) | | | TCCAAATGGGACCTCTGGCTA′] |
| 3298 | NM_000019.3(ACAT1): c.278A > G | 38 | ACAT1 | [′GAAGTGAAAGAAGCATACATGGGTARTGT |
| | (p.Asn93Ser) | | | TCTACAAGGAGGTGAAGGACAA′] |
| 3299 | NM_173076.2(ABCA12): | 26154 | ABCA12 | [′CATATTACTTCATTGCTGGGGCCCARTGGA |
| | c.4139A > G (p.Asn1380Ser) | | | GCTGGGAAAACTACTACCATG′] |
| 3300 | NM_030665.3(RAI1): c.4685A > G | 10743 | RAI1 | [′AAGGGGCGTGCCAAGCGACGACGACRGCA |
| | (p.Gln1562Arg) | | | GCAGGTGCTGCCCCTGGATCCC′] |
| 3301 | NM_000271.4(NPC1): c.3467A > G | 4864 | NPC1 | [′AGTCTGAACGCTGTATCCTTGGTCARCCTG |
| | (p.Asn1156Ser) | | | GTGATGGTGAGTCCTCATACA′] |
| 3302 | NM_000271.4(NPC1): c.3263A > G | 4864 | NPC1 | [′CCCTCCAGTGTGTTTTATGTCTTCTRCGAA |
| | (p.Tyr1088Cys) | | | CAGTACCTGACCATCATTGAC′] |
| 3303 | NM_000543.4(SMPD1): | 6609 | SMPD1 | [′CTCCGCCTCATCTCTCTCAATATGARTTTTT |
| | c.1154A > G (p.Asn385Ser) | | | GTTCCCGTGAGAACTTCTGG′] |
| 3304 | NM_000226.3(KRT9): c.469A > G | 3857 | KRT9 | [′TCTGACTGCTAATGAGAAGAGCACCRTGC |
| | (p.Met157Val) | | | AGGAACTCAATTCTCGGCTGGC′] |
| 3305 | NM_000226.3(KRT9): c.482A > G | 3857 | KRT9 | [′GAGAAGAGCACCATGCAGGAACTCADTTC |
| | (p.Asn161Ser) | | | TCGGCTGGCCTCTTACTTGGAT′] |
| 3306 | NM_176824.2(BBS7): c.968A > G | 55212 | BBS7 | [′ACAGGTCTGACAACAGAGCCCATTCRTAA |
| | (p.His323Arg) | | | GGAAAGTGGACCAGGAGAAGAA′] |
| 3307 | NM_000051.3(ATM): c.3118A > G | 472 | ATM | [′GAGGAAATATATATTCTCTGTAAGARTGG |
| | (p.Met1040Val) | | | CCCTAGTAAATTGCCTTAAAAC′] |
| 3308 | NM_000051.3(ATM): c.7268A > G | 472 | ATM | [′GCTCTCCTGAAAAGAGCCAAAGAGGRAGT |
| | (p.Glu2423Gly) | | | AGGTCTCCTTAGGGAACATAAA′] |
| 3309 | NM_000051.3(ATM): c.8030A > G | −1 | — | [′TATTAGGTGGACCACACAGGAGAATRTGG |
| | (p.Tyr2677Cys) | | | AAATCTGGTGACTATACAGTCA′] |
| 3310 | NM_000487.5(ARSA): | 410 | ARSA | [′GAGGGGGTTTGTGCCTGATAACGTARTAA |
| | c.*96A > G | | | CACCAGTGGAGACTTGCAGATG′] |
| 3311 | NM_000487.5(ARSA): c.1055A > G | 410 | ARSA | [′GCCCTGGCTGGGGCCCCACTGCCCARTGTC |
| | (p.Asn352Ser) | | | ACCTTGGATGGCTTTGACCTC′] |
| 3312 | NM_000218.2(KCNQ1): c.418A > G | 3784 | KCNQ1 | [′CATCGTCCTGGTCTGCCTCATCTTCRGCGT |
| | (p.Ser140Gly) | | | GCTGTCCACCATCGAGCAGTA′] |
| 3313 | NM_172250.2(MMAA): c.620A > G | 166785 | MMAA | [′GAGTTATCAAGAGATATGAATGCATRCAT |
| | (p.Tyr207Cys) | | | CAGGCCATCTCCTACTAGAGGA′] |
| 3314 | NM_000187.3(HGD): c.1112A > G | 3081 | HGD | [′AGTCTACACAGCACAATGACCCCCCRTGG |
| | (p.His371Arg) | | | ACCTGATGCTGACTGCTTTGAG′] |
| 3315 | NM_000187.3(HGD): c.1102A > G | 3081 | HGD | [′AGGGGGAGGGAGTCTACACAGCACARTGA |
| | (p.Met368Val) | | | CCCCCCATGGACCTGATGCTGA′] |
| 3316 | NM_138477.2(CDAN1): | 146059 | CDAN1 | [′CTGAGCTTGAAGATCCAGGAGCTCARTGG |
| | c.1796A > G (p.Asn599Ser) | | | TCTTGCCCTGCCCCAGCATGAG′] |
| 3317 | NM_001079802.1(FKTN): | 2218 | FKTN | [′GTAAAACTTGATGTTTTTTTCTTCTRTGAA |
| | c.1112A > G (p.Tyr371Cys) | | | GAAACTGATCACATGTGGAAT′] |
| 3318 | NM_013382.5(POMT2): | 29954 | POMT2 | [′GCTTGAGAAGAGCATGGCTGGGAAGYAGT |
| | c.1997A > G (p.Tyr666Cys) | | | GGTGGAAGTAGAGGACCCGGCC′] |
| 3319 | NM_000529.2(MC2R): c.761A > G | 4158 | MC2R | [′CCAAGTAACCCCTACTGCGCCTGCTRCATG |
| | (p.Tyr254Cys) | | | TCTCTCTTCCAGGTGAACGGC′] |
| 3320 | NM_000383.3(AIRE): c.247A > G | 326 | AIRE | [′CCTGGACTTCTGGAGGGTGCTGTTCRAGGA |
| | (p.Lys83Glu) | | | CTACAACCTGGAGCGCTATGG′] |
| 3321 | NM_020436.3(SALL4): | 57167 | SALL4 | [′TCGTCTGCTAGCGCTCTTCAGATCCRCGAG |
| | c.2663A > G (p.His888Arg) | | | CGGACTCACACTGGAGAGAAG′] |
| 3322 | NM_203288.1(RP9): c.509A > G | 6100 | RP9 | [′CAGTTACTGGAGGATTCTACCTCAGRTGAA |
| | (p.Asp170Gly) | | | GATAGGAGCAGCTCCAGTTCC′] |
| 3323 | NM_000348.3(SRD5A2): | 6716 | SRD5A2 | [′TTCCTTGGGCTGCGAGCTTTTCACCRCCAT |
| | c.692A > G (p.His231Arg) | | | AGGTAAATTTTTCAATAAAAG′] |
| 3324 | NM_006445.3(PRPF8): | 10594 | PRPF8 | [′CCCAAAGAGTTCTACCACGAGGTGCVCAG |
| | c.6926A > G (p.His2309Arg) | | | GCCCTCTCACTTCCTCAACTTT′] |
| 3325 | NM_018319.3(TDP1): c.1478A > G | 55775 | TDP1 | [′TCTGGCCGCAGCAATGCCATGCCACRTATT |
| | (p.His493Arg) | | | AAGACATATATGAGGCCTTCT′] |
| 3326 | NM_144773.2(PROKR2): | 128674 | PROKR2 | [′AGCCAGGAGAAGATCTTCTGTGGCCRGAT |
| | c.629A > G (p.Gln210Arg) | | | CTGGCCTGTGGATCAGCAGCTC′] |
| 3327 | NM_001604.5(PAX6): | 5080 | PAX6 | [′ACCACAGGTTTGCCTCTCTCCTCACRGCCC |
| | c.1075 − 2A > G | | | CCAGTCCCCAGCCAGACCTCC′] |
| 3328 | NM_000280.4(PAX6): c.1171A > G | 5080 | PAX6 | [′CAGTCAGCCAATGGGCACCTCGGGCRCCA |
| | (p.Thr391Ala) | | | CTTCAACAGGTGAGCCACTGCT′] |
| 3329 | NM_024426.4(WT1): c.1391A > G | 7490 | WT1 | [′TGTCAGCGAAAGTTCTCCCGGTCCGRCCAC |
| | (p.Asp464Gly) | | | CTGAAGACCCACACCAGGACT′] |
| 3330 | NM_024426.4(WT1): c.1021A > G | 7490 | WT1 | [′TTGCAGCCACAGCACAGGGTACGAGRGCG |
| | (p.Ser341Gly) | | | ATAACCACACAACGCCCATCCT′] |
| 3331 | NM_005957.4(MTHFR): c.971A > G | 4524 | MTHFR | [′CCAGGCCTCCACTTCTACACCCTCARCCGC |
| | (p.Asn324Ser) | | | GAGATGGCTACCACAGAGGTG′] |
| 3332 | NM_001127328.2(ACADM): | 34 | ACADM | [′TAACTCATTCTAGCTAGTTCAACTTYCATT |
| | c.997A > G (p.Lys333Glu) | | | GCCATTTCAGCCAGCATAAAT′] |
| 3333 | NM_001127328.2(ACADM): | 34 | ACADM | [′TATTAATGGTCAGAAGATGTGGATARCCA |
| | c.589A > G (p.Thr197Ala) | | | ACGGAGGAAAAGCTAATTGGTA′] |
| 3334 | NM_000155.3(GALT): c.940A > G | 2592 | GALT | [′AGGATCAGAGGCTGGGGCCAACTGGRACC |
| | (p.Asn314Asp) | | | ATTGGCAGCTGCACGCTCATTA′] |
| 3335 | NM_000155.3(GALT): c.563A > G | 2592 | GALT | [′TGTTCTAACCCCCACCCCCACTGCCRGGTA |
| | (p.Gln188Arg) | | | AGGGTGTCAGGGGCTCCAGTG′] |
| 3336 | NM_000250.1(MPO): c.518A > G | 4353 | MPO | [′GTTGCACATCCCGGTGATGGTGCGGYATTT |
| | (p.Tyr173Cys) | | | GTCCTGCTCCGGGCAAGTCAC′] |
| 3337 | NM_020247.4(ADCK3): | 56997 | ADCK3 | [′TTGGATTTTGGGGCAACGCGGGAATRTGA |
| | c.1541A > G (p.Tyr514Cys) | | | CAGATCCTTCACCGACCTCTAC′] |
| 3338 | NM_000229.1(LCAT): c.463A > G | 3931 | LCAT | [′CACACTGGTGCAGAACCTGGTCAACRATG |
| | (p.Asn155Asp) | | | GCTACGTGCGGGACGAGACTGT′] |
| 3339 | NM_000403.3(GALE): c.101A > G | 2582 | GALE | [′GGCTACTTGCCTGTGGTCATCGATARCTTC |
| | (p.Asn34Ser) | | | CATAATGCCTTCCGTGGTGAG′] |
| 3340 | NM_001008216.1(GALE): | 2582 | GALE | [′GGCGAGTCGGTGCAGAAGCCTCTGGRTTA |
| | c.308A > G (p.Asp103Gly) | | | TTACAGAGTTAACCTGACCGGG′] |
| 3341 | NM_000403.3(GALE): c.770A > G | 2582 | GALE | [′AAGGGCCACATTGCAGCCTTAAGGARGCT |
| | (p.Lys257Arg) | | | GAAAGAACAGTGTGGCTGCCGG′] |
| 3342 | NM_000527.4(LDLR): c.2483A > G | 3949 | LDLR | [′AGCATCAACTTTGACAACCCCGTCTRTCAG |
| | (p.Tyr828Cys) | | | AAGACCACAGAGGATGAGGTC′] |
| 3343 | NM_024740.2(ALG9): c.860A > G | 79796 | ALG9 | [′ATTGCACCACTCAACATTGTTTTGTRTAAT |
| | (p.Tyr287Cys) | | | GTCTTTACTCCTCATGGACCT′] |
| 3344 | NM_000375.2(UROS): c.184A > G | 7390 | UROS | [′TGAAGATTACGGGGGACTCATTTTTRCCAG |
| | (p.Thr62Ala) | | | CCCCAGAGCAGTGGAAGCAGC′] |
| 3345 | NM_000372.4(TYR): c.125A > G | 7299 | TYR | [′GAATGCTGTCCACCGTGGAGCGGGGRCAG |
| | (p.Asp42Gly) | | | GAGTCCCTGTGGCCAGCTTTCA′] |
| 3346 | NM_000372.4(TYR): c.1A > G | 7299 | TYR | [′AGACCTTGTGAGGACTAGAGGAAGARTGC |
| | (p.Met1Val) | | | TCCTGGCTGTTTTGTACTGCCT′] |
| 3347 | NM_000053.3(ATP7B): | 540 | ATP7B | [′GTCGCCATGGTGGGGGATGGGGTCADTGA |
| | c.3809A > G (p.Asn1270Ser) | | | CTCCCCGGCCTTGGCCCAGGCA′] |
| 3348 | NM_000520.4(HEXA): c.1A > G | 3073 | HEXA | [′CTCCGAGAGGGGAGACCAGCGGGCCVTGA |
| | (p.Met1Val) | | | CAAGCTCCAGGCTTTGGTTTTC′] |
| 3349 | NM_000520.4(HEXA): c.611A > G | 3073 | HEXA | [′AATAAATTGAACGTGTTCCACTGGCRTCTG |
| | (p.His204Arg) | | | GTAGATGATCCTTCCTTCCCA′] |
| 3350 | NM_000356.3(TCOF1): c.149A > G | 6949 | TCOF1 | [′CAGCCCGTAACCCTTCTGGACATCTRTACA |
| | (p.Tyr50Cys) | | | CACTGGCAACAGTAAGTGGTG′] |
| 3351 | NM_000144.4(FXN): | 2395 | FXN | [′ATGCTTTTTTTCCACCTAATCCCCTRGAGT |
| | c.38 − 2A > G | | | GGTGTCTTAACTGTCAAACTG′] |
| 3352 | NM_016335.4(PRODH): | 5625 | PRODH | [′GCCTAGCAGCTGTCCAAAGTACACCYGGT |
| | c.1562A > G (p.Gln521Arg) | | | GGTCAGCAGGATGCAGGCCCAG′] |
| 3353 | NM_015474.3(SAMHD1): | 25939 | SAMHD1 | [′TAATTCTAATGGAATTAAGCCTGTCRTGGA |
| | c.760A > G (p.Met254Val) | | | ACAATATGGTCTCATCCCTGA′] |
| 3354 | NM_012464.4(TLL1): c.1885A > G | 7092 | TLL1 | [′ACTTCTTACCAAACTTAACGGCACCRTAAC |
| | (p.Ile629Val) | | | CACCCCTGGCTGGCCCAAGGA′] |
| 3355 | NM_000112.3(SLC26A2): | 1836 | SLC26A2 | [′GGAAATGTATGCCATTGGCTTTTGTRATAT |
| | c.1273A > G (p.Asn425Asp) | | | CATCCCTTCCTTCTTCCACTG′] |
| 3356 | NM_138691.2(TMC1): c.1960A > G | 117531 | TMC1 | [′CCTGTCCACAATGCCTGTCTTGTACRTGAT |
| | (p.Met654Val) | | | CGTGTCCCTCCCACCATCTTT′] |
| 3357 | NM_000173.6(GP1BA): c.763A > G | 2811 | GP1BA | [′GAAGCAAGGTGTGGACGTCAAGGCCRTGA |
| | (p.Met255Val) | | | CCTCTAATGTGGCCAGTGTGCA′] |
| 3358 | NM_024996.5(GFM1): c.521A > G | 85476 | GFM1 | [′AACGTTCCGTTTCTAACTTTTATTARCAAA |
| | (p.Asn174Ser) | | | TTGGACCGAATGGGCTCCAAC′] |
| 3359 | NM_024301.4(FKRP): c.926A > G | 79147 | FKRP | [′GTGGGCGACACGCCCGCCTACCTCTRCGA |
| | (p.Tyr309Cys) | | | GGAGCGCTGGACGCCCCCCTGC′] |
| 3360 | NM_024301.4(FKRP): c.1387A > G | 79147 | FKRP | [′TGCCGGCTTCGTGGCGCAGGCGCCTRACA |
| | (p.Asn463Asp) | | | ACTACCGCCGCTTCCTGGAGCT′] |
| 3361 | NM_021020.3(LZTS1): c.355A > G | 11178 | LZTS1 | [′TCCTTTTCCTCTACAGGGCTCCGAGRAGGG |
| | (p.Lys119Glu) | | | TGCAGTGAGGCCCACAGCCTT′] |
| 3362 | NM_001005741.2(GBA): | 2629 | GBA | [′CCAGCCGACCACATGGTACAGGAGGBTCT |
| | c.1226A > G (p.Asn409Ser) | | | AGGGTAAGGACAAAGGCAAAGA′] |
| 3363 | NM_000157.3(GBA): c.680A > G | 2629 | GBA | [′ACATCACCCACTTGGCTCAAGACCARTGG |
| | (p.Asn227Ser) | | | AGCGGTGAATGGGAAGGGGTCA′] |
| 3364 | NM_001005741.2(GBA): | 2629 | GBA | [′AAATATGTTCATGGCATTGCTGTACRTTGG |
| | c.1049A > G (p.His350Arg) | | | TACCTGGACTTTCTGGCTCCA′] |
| 3365 | NM_014239.3(EIF2B2): | 8892 | EIF2B2 | [′GTGAATTTGTCCAAAGCAGGTATTGRGAC |
| | c.638A > G (p.Glu213Gly) | | | AACTGTCATGACTGATGCTGCC′] |
| 3366 | NM_015915.4(ATL1): c.773A > G | 51062 | ATL1 | [′CTACAGAACGTCAGAAAACACATCCRTTC |
| | (p.His258Arg) | | | CTGTTTCACCAACATTTCCTGT′] |
| 3367 | NM_015915.4(ATL1): c.1222A > G | 51062 | ATL1 | [′GAAGCTATTCCGAGGGGTGAAGAAGRTGG |
| | (p.Met408Val) | | | GTGGGGAAGAATTTAGCCGGCG′] |
| 3368 | NM_022051.2(EGLN1): | 54583 | EGLN1 | [′TTCTGGTCTGACCGTCGCAACCCTCRTGAA |
| | c.1121A > G (p.His374Arg) | | | GTACAACCAGCATATGCTACA′] |
| 3369 | NM_015629.3(PRPF31): | 26121 | PRPF31 | [′GTCACCGCCTCCACCACCCAGGGGTRTGTC |
| | c.527 + 3A > G | | | CGCTTCGAGGGAGGCGCCGGG′] |
| 3370 | NM_001243133.1(NLRP3): | 114548 | NLRP3 | [′CTGCAGATCCAGCCCAGCCAGCTGGRATT |
| | c.1880A > G (p.Glu627Gly) | | | GTTCTACTGTTTGTACGAGATG′] |
| 3371 | NM_020919.3(ALS2): | 57679 | ALS2 | [′CTTATTGACGTTACTTTTTGCTCCTRGACA |
| | c.2980 − 2A > G | | | AAGTGGCTACGAGCTATAAGC′] |
| 3372 | NM_175073.2(APTX): c.602A > G | 54840 | APTX | [′GATAAATACCCAAAGGCCCGTTACCRTTG |
| | (p.His201Arg) | | | GCTGGTCTTACCGTGGACCTCC′] |
| 3373 | NM_001002010.2(NT5C3A): | 51251 | NT5C3A | [′CATCCCAATGTCAAAGTTGTGTCCARTTTT |
| | c.686A > G (p.Asn229Ser) | | | ATGGATTTTGATGAAACTGTA′] |
| 3374 | NM_020451.2(SEPN1): c.1A > G | 57190 | SEPN1 | [′CCGGCAGCCGCCGCCAGCCGCAGCCRTGG |
| | (p.Met1Val) | | | GCCGGGCCCGGCCGGGCCAACG′] |
| 3375 | NM_032667.6(BSCL2): c.263A > G | −1 | — | [′TCACTCTGCTCCTTCCCTGTTGCCARTGTCT |
| | (p.Asn88Ser) | | | CGCTGACTAAGGGTGGACGT′] |
| 3376 | NM_153638.2(PANK2): c.700A > G | 80025 | PANK2 | [′GGTATATTTTGAACCCAAAGACATCRCTGC |
| | (p.Thr234Ala) | | | TGAAGAAGAAGAGGAAGAAGT′] |
| 3377 | NM_025243.3(SLC19A3): | 80704 | SLC19A3 | [′TGCCTTGGTGATTCAGACCATCATGRCTGT |
| | c.1264A > G (p.Thr422Ala) | | | GATTGTAGTAGATCAGAGAGG′] |
| 3378 | NM_025243.3(SLC19A3): | 80704 | SLC19A3 | [′TATCCCATATTTATCTGGACCAGATRAAAA |
| | c.130A > G (p.Lys44Glu) | | | CCTGACCAGTGCAGAGGTAAG′] |
| 3379 | NM_031885.3(BBS2): c.209A > G | 583 | BBS2 | [′CAGACAGCTGACTGCCTGGTTAATGYTGA |
| | (p.Asn70Ser) | | | GAAGAGAAACATCAGATTCCAG′] |
| 3380 | NM_032383.4(HPS3): | −1 | — | [′TTTATGAGAAATTCTTTTATGTTTTRGATA |
| | c.2482 − 2A > G | | | AATGCCTGTAGTCATTATGGC′] |
| 3381 | NM_021830.4(C10orf2): | 56652 | C10orf2 | [′TGACCTTCGGTCCTGGGAAGCCGCCVAGTT |
| | c.955A > G (p.Lys319Glu) | | | GTTTGCACGAAAACTGAACCC′] |
| 3382 | NM_021830.4(C10orf2): | 56652 | C10orf2 | [′ACAATGCAACATGCAGTCTACGTCTRTGAC |
| | c.1523A > G (p.Tyr508Cys) | | | ATTTGTCATGTGATCATCGAC′] |
| 3383 | NM_006492.2(ALX3): c.608A > G | 257 | ALX3 | [′ACCTGCCCCCAGGTCTGGTTCCAGARCCGC |
| | (p.Asn203Ser) | | | AGAGCCAAGTGGCGGAAGCGC′] |
| 3384 | NM_174916.2(UBR1): c.407A > G | 197131 | UBR1 | [′TTCCAGGACAGTGTTCATAAAAATCRTCGT |
| | (p.His136Arg) | | | TACAAGGTAAGAAAATATAAC′] |
| 3385 | NM_015166.3(MLC1): c.422A > G | 23209 | MLC1 | [′CTAGTCCTGAACCCATCAGCAATAARCGT |
| | (p.Asn141Ser) | | | GAGTTCACACGAGCTTCGCGCA′] |
| 3386 | NM_013391.3(DMGDH): | 29958 | DMGDH | [′CCTGGAATAAACTTGAAGAAAATACRTTA |
| | c.326A > G (p.His109Arg) | | | TGATAGCATCAAACTTTATGAG′] |
| 3387 | NM_014795.3(ZEB2): c.3356A > G | 9839 | ZEB2 | [′GCTTACTTGCAGAGCATTACCCCTCRGGGG |
| | (p.Gln1119Arg) | | | TACTCTGACTCGGAGGAGAGG′] |
| 3388 | NM_000441.1(SLC26A4): | 5172 | SLC26A4 | [′GTTGTCATCCAGTCTCTTCCTTAGGRATTC |
| | c.1151A > G (p.Glu384Gly) | | | ATTGCCTTTGGGATCAGCAAC′] |
| 3389 | NM_000441.1(SLC26A4): | 5172 | SLC26A4 | [′TTATGCTATTGCAGTGTCAGTAGGARAAGT |
| | c.1105A > G (p.Lys369Glu) | | | ATATGCCACCAAGTATGATTA′] |
| 3390 | NM_000441.1(SLC26A4): | 5172 | SLC26A4 | [′AAGGACACATTCTTTTTGACGGTCCRTGAT |
| | c.2168A > G (p.His723Arg) | | | GCTATACTCTATCTACAGAAC′] |
| 3391 | NM_000441.1(SLC26A4): | 5172 | SLC26A4 | [′TTTTTAACATCTTTTGTTTTATTTCRGACGA |
| | c.919 − 2A > G | | | TAATTGCTACTGCCATTTCA′] |
| 3392 | NM_000197.1(HSD17B3): | 3293 | HSD17B3 | [′GACCCCATATGCTGTCTCGACTGCARTGAC |
| | c.703A > G (p.Met235Val) | | | AAAGTATCTAAATACAAATGT′] |
| 3393 | NM_000197.1(HSD17B3): | 3293 | HSD17B3 | [′TTGTTTTGCTTCTTTCTGCCAGTCARCAATG |
| | c.389A > G (p.Asn130Ser) | | | TCGGAATGCTTCCAAACCTT′] |
| 3394 | NM_022458.3(LMBR1): | 64327 | LMBR1 | [′GTTTTGATCTTAGTGTTTATTACAGRAAAT |
| | c.423 + 5252A > G | | | GAAGCCATATCTCACTAACTA′] |
| 3395 | NM_020366.3(RPGRIP1): | 57096 | RPGRIP1 | [′CTAAATAAACATTTTCCTTATCAGGRTTCA |
| | c.3341A > G (p.Asp1114Gly) | | | GAGAAGATGTGCATTGAAATT′] |
| 3396 | NM_021625.4(TRPV4): c.998A > G | 59341 | TRPV4 | [′CTGCATGCGCTGGTGGCCATTGCTGRCAAC |
| | (p.Asp333Gly) | | | ACCCGTGAGAACACCAAGTTT′] |
| 3397 | NM_004897.4(MINPP1): | 9562 | MINPP1 | [′TTAAAAAAAGTTGCAGCTACTTTGCRAGTG |
| | c.809A > G (p.Gln270Arg) | | | CCAGTAAATGATTTAAATGCA′] |
| 3398 | NM_020638.2(FGF23): c.211A > G | 8074 | FGF23 | [′TGGCGCACCCCATCAGACCATCTACRGTG |
| | (p.Ser71Gly) | | | AGTAGGGCTTCAGGCTGGGAAG′] |
| 3399 | NM_015071.4(ARHGAP26): | 23092 | ARHGAP26 | [′GGGCTGTATCGAATTGTGGGTGTCAVCTCC |
| | c.1250A > G (p.Asn417Ser) | | | AGAGTGCAGAAGTTGCTGAGT′] |
| 3400 | NM_021615.4(CHST6): c.521A > G | 4166 | CHST6 | [′CGCTCCTACAGCCACGTGGTGCTCARGGA |
| | (p.Lys174Arg) | | | GGTGCGCTTCTTCAACCTGCAG′] |
| 3401 | NM_021615.4(CHST6): c.329A > G | 4166 | CHST6 | [′TGCGACATGGACGTGTTTGATGCCTRTCTG |
| | (p.Tyr110Cys) | | | CCTTGGCGCCGCAACCTGTCC′] |
| 3402 | NM_015560.2(OPA1): c.1745A > G | 4976 | OPA1 | [′CTTGAAACTGAATGGAAGAATAACTRTCC |
| | (p.Tyr582Cys) | | | TCGCCTGCGGGAACTTGACCGG′] |
| 3403 | NM_000368.4(TSC1): c.1760A > G | 7248 | TSC1 | [′AGTATCTTCACTCCCAGTCCTTGTARAATT |
| | (p.Lys587Arg) | | | CCACCTCCGACGAGAGTGGGC′] |
| 3404 | NM_020661.2(AICDA): c.415A > G | 57379 | AICDA | [′CCGCGCCGGGGTGCAAATAGCCATCRTGA |
| | (p.Met139Val) | | | CCTTCAAAGGTGCGAAAGGGCC′] |
| 3405 | NM_020533.2(MCOLN1): | 57192 | MCOLN1 | [′ACAGGCCCTCCCCTTCTCTGCCCACRGTAC |
| | c.406 − 2A > G | | | CTGGCGTTGCCTGACGTGTCA′] |
| 3406 | NM_021102.3(SPINT2): | 10653 | SPINT2 | [′AGGAACTCCTGCAATAACTTCATCTRTGGA |
| | c.488A > G (p.Tyr163Cys) | | | GGCTGCCGGGGCAATAAGAAC′] |
| 3407- | NM_006610.3(MASP2): c.359A > G | 10747 | MASP2 | [′GGGAGGGCTGGGGTTTCTCAGGGTCRGGG |
| 3410 | (p.Asp120Gly) | | | GGTCCCCAAGGAGTAGCCAGGG′, |
| | | | | ′AGGGGCCAGGCTTGGCCAGGAGGGASATC |
| | | | | AGGCCTGGGTCTTGCCTTCACT′, |
| | | | | ′GGGCAGGAGAGCACAGACACGGAGCRGGC |
| | | | | CCCTGGCAAGGACACTTTCTAC′, |
| | | | | ′AGCCTGGACATTACCTTCCGCTCCGRCTAC |
| | | | | TCCAACGAGAAGCYGTTCACG′] |
| 3411 | NM_018965.3(TREM2): c.401A > G | 54209 | TREM2 | [′GCTCTGCCTCCCATAGACCCCCTGGRTCAC |
| | (p.Asp134Gly) | | | CGGGATGCTGGAGATCTCTGG′] |
| 3412 | NM_007375.3(TARDBP): | 23435 | TARDBP | [′AGCACTACAGAGCAGTTGGGGTATGRTGG |
| | c.1009A > G (p.Met337Val) | | | GCATGTTAGCCAGCCAGCAGAA′] |
| 3413 | NM_007375.3(TARDBP): | 23435 | TARDBP | [′GTAATGTCACAGCGACATATGATAGRTGG |
| | c.506A > G (p.Asp169Gly) | | | ACGATGGTGTGACTGCAAACTT′] |
| 3414 | NM_007375.3(TARDBP): | 23435 | TARDBP | [′GGTATGATGGGCATGTTAGCCAGCCRGCA |
| | c.1028A > G (p.Gln343Arg) | | | GAACCAGTCAGGCCCATCGGGT′] |
| 3415 | NM_007375.3(TARDBP): | 23435 | TARDBP | [′CGTTCATATATCCAATGCCGAACCTRAGCA |
| | c.787A > G (p.Lys263Glu) | | | CAATAGCAATAGACAGTTAGA′] |
| 3416 | NM_015247.2(CYLD): c.2240A > G | 1540 | CYLD | [′ATCAACAGTAACCTGAAATTTGCAGRGGT |
| | (p.Glu747Gly) | | | TAGTGATACTCACCTGTGGTAT′] |
| 3417 | NM_001128425.1(MUTYH): | 4595 | MUTYH | [′AATCAACTCTGGGCTGGCCTGGGCTRCTAT |
| | c.536A > G (p.Tyr179Cys) | | | TCTCGTGGCCGGCGGCTGCAG′] |
| 3418 | NM_001128425.1(MUTYH): | 4595 | MUTYH | [′TCCGTGACCTGGGAGCCCTCAGAGCRGCTT |
| | c.1241A > G (p.Gln414Arg) | | | CAGCGCAAGGCCCTGCTGCAG′] |
| 3419 | NM_170784.2(MKKS): c.110A > G | 8195 | MKKS | [′TTGAAAAGAATTGTAACATCATGCTRTGGC |
| | (p.Tyr37Cys) | | | CCCTCAGGTAGGCTGAAGCAG′] |
| 3420 | NM_170784.2(MKKS): c.169A > G | 8195 | MKKS | [′TGGCTTTGGAGGTTACGTGTGTACARCCTC |
| | (p.Thr57Ala) | | | ACAGTCCTCAGCTCTGCTCAG′] |
| 3421 | NM_004795.3(KL): c.578A > G | 9365 | KL | [′GTGCAGCCCGTGGTCACCCTGTACCRCTGG |
| | (p.His193Arg) | | | GACCTGCCCCAGCGCCTGCAG′] |
| 3422 | NM_014625.3(NPHS2): c.479A > G | 7827 | NPHS2 | [′CTTTTCTTTTTTTTGCCCTGCCTGGRTACCT |
| | (p.Asp160Gly) | | | ACCACAAGGTTGACCTTCGT′] |
| 3423 | NM_015713.4(RRM2B): | 50484 | RRM2B | [′CACCTCCTGACTAAAGCGCTCCACCBAAG |
| | c.322 − 2A > G | | | AAGATAAGGAAAATAGATATAT′] |
| 3424 | NM_006343.2(MERTK): | 10461 | MERTK | [′GAAGTATCTTTGTTTTCATTCACCCRGGAA |
| | c.1605 − 2A > G | | | TGCATTCACAGAGGAGGATTC′] |
| 3425 | NM_014585.5(SLC40A1): | 30061 | SLC40A1 | [′GCTACTGCAATCACAATCCAAAGGGRTTG |
| | c.470A > G (p.Asp157Gly) | | | GATTGTTGTTGTTGCAGGAGAA′] |
| 3426 | NM_004924.4(ACTN4): c.763A > G | 81 | ACTN4 | [′CGTGAACACGGCCCGGCCCGACGAGRAGG |
| | (p.Lys255Glu) | | | CCATAATGACCTATGTGTCCAG′] |
| 3427 | NM_012193.3(FZD4): c.1024A > G | −1 | — | [′CAAATGGGGTCATGAAGCCATTGAARTGC |
| | (p.Met342Val) | | | ACAGCTCTTATTTCCACATTGC′] |
| 3428 | NM_012193.3(FZD4): c.766A > G | −1 | — | [′GTTTTCCTACCCTGAGCGCCCCATCRTATT |
| | (p.Ile256Val) | | | TCTCAGTATGTGCTATAATAT′] |
| | NM_004239.3(TRIP11): | 9321 | TRIP11 | |
| | c.202 − 2A > G | | | |
| 3429 | NM_004239.3(TRIP11): | 9321 | TRIP11 | [′AGTGTTTTTTTCCAGAGAAAGCTGAYTGTT |
| | c.2102A > G (p.Asn701Ser) | | | ACCAGCAAGACATTCTTCTAA′] |
| 3430 | NM_012434.4(SLC17A5): | 26503 | SLC17A5 | [′CAGGGTGTTACATTTCCAGCCATGCRTGCC |
| | c.548A > G (p.His183Arg) | | | ATGTGGTCTTCTTGGGCTCCC′] |
| 3431 | NM_001099274.1(TINF2): | 26277 | TINF2 | [′ATGGGCCTCCACTAGGGGAGGCCATDAGG |
| | c.838A > G (p.Lys280Glu) | | | AGCGCCCCACAGTCATGCTGTT′] |
| 3432 | NM_012415.3(RAD54B): | 25788 | RAD54B | [′AGAGTTGAACAAAAGGCAGGGGTGAYTGC |
| | Gc.1778A > (p.Asn593Ser) | | | ACAGTTTTTTAAGAGCTCCTAT′] |
| 3433 | NM_014946.3(SPAST): | 6683 | SPAST | [′TAAGTGCCTGACTTTTATGTTTTACRGAAC |
| | c.1688 − 2A > G | | | TAAAACCAGAACAGGTGAAGA′] |
| 3434 | NM_014946.3(SPAST): | 6683 | SPAST | [′AGTCTTATACTTGTATTTCCTCTAGRTGAA |
| | c.1322A > G (p.Asp441Gly) | | | GTTGATAGCCTTTTGTGTGAA′] |
| 3435 | NM_014946.3(SPAST): | 6683 | SPAST | [′TGCAAGTTTAACTTCAAAATACGTGRGTGC |
| | c.1245 + 4A > G | | | TCTGTTTCCAATATTGTCGTA′] |
| 3436 | NM_014946.3(SPAST): | 6683 | SPAST | [′CTGTTACTCTTTGGTCCACCTGGGARTGGG |
| | c.1157A > G (p.Asn386Ser) | | | AAGACAATGCTGGTAAGGGTT′] |
| 3437 | NM_014946.3(SPAST): | 6683 | SPAST | [′AGAATCGAATGCAACCTTCTTTAATRTAAG |
| | c.1216A > G (p.Ile406Val) | | | TGCTGCAAGTTTAACTTCAAA′] |
| 3438 | NM_182643.2(DLC1): c.2875A > G | 10395 | DLC1 | [′ACACCTGGATGTGGACAACGACCGARCCA |
| | (p.Thr959Ala) | | | CACCCAGCGACCTGGACAGCAC′] |
| 3439 | NM_194456.1(KRIT1): c.410A > G | 889 | KRIT1 | [′TGCCCAATTTTTTACTGCTTACAAGRTATT |
| | (p.Asp137Gly) | | | ATGCGAGTCTGTAGTGAATCC′] |
| 3440 | NM_005094.3(SLC27A4): | 10999 | SLC27A4 | [′CCAGGAAACATCGTGGGAATCGGCCRGTG |
| | c.899A > G (p.Gln300Arg) | | | CCTGCTGCATGGCATGACGGTG′] |
| 3441 | NM_004211.3(SLC6A5): | 9152 | SLC6A5 | [′GGAGGCCTGATCACTCTCTCTTCTTRCAAC |
| | c.1472A > G (p.Tyr491Cys) | | | AAATTCCACAACAACTGCTAC′] |
| 3442 | NM_004211.3(SLC6A5): | 9152 | SLC6A5 | [′GACACTCTAATTGTCACCTGCACCARCAGT |
| | c.1526A > G (p.Asn509Ser) | | | GCCACAAGCATCTTTGCCGGC′] |
| 3443 | NM_014270.4(SLC7A9): | 11136 | SLC7A9 | [′GCGTTTTACAATGGACTCTGGGCCTRTGAT |
| | c.695A > G (p.Tyr232Cys) | | | GGATGGTGAGGTGTCCTGAGA′] |
| 3444 | NM_139025.4(ADAMTS13): | 11093 | ADAMTS13 | [′GAGCCTGTGTGTGTCGGGCAGCTGCRGGG |
| | c.1582A > G (p.Arg528Gly) | | | TAGGCGTGTGTGGACATTGGCG′] |
| 3445 | NM_002894.2(RBBP8): | 5932 | RBBP8 | [′TGTATTTGGAGCTACCTCTAGTATCRAAAG |
| | c.1009A > G (p.Lys337Glu) | | | TGGTTTAGATTTGAATACAAG′] |
| 3446 | NM_005682.6(ADGRG1): | 9289 | ADGRG1 | [′TCCTTCCCTGACCCCAGGGGCCTCTRCCAC |
| | Gc.263A > (p.Tyr88Cys) | | | TTCTGCCTCTACTGGAACCGA′] |
| 3447 | NM_006502.2(POLH): c.1603A > G | 5429 | POLH | [′AGGAACTGAGCCCTTCTTTAAGCAGRAAA |
| | (p.Lys535Glu) | | | GTCTGCTTCTAAAGCAGAAACA′] |
| 3448 | NM_000334.4(SCN4A): | 6329 | SCN4A | [′CATCTCCTTCCTCATCGTGGTCAACRTGTA |
| | c.4774A > G (p.Met1592Val) | | | CATCGCCATCATCCTGGAGAA′] |
| 3449 | NM_000334.4(SCN4A): | 6329 | SCN4A | [′CGCCCTCCTAGGCGCCATCCCCTCCRTCAT |
| | c.3478A > G (p.Ile1160Val) | | | GAATGTGCTGCTTGTCTGCCT′] |
| 3450 | NM_000334.4(SCN4A): c.421A > G | 6329 | SCN4A | [′GTTCAGCATGTTCATCATGATCACCRTCTT |
| | (p.Ile141 Val) | | | GACCAACTGCGTATTCATGAC′] |
| 3451 | NM_003907.2(EIF2B5): | 8893 | EIF2B5 | [′CCTGACTGCCACAGGTGTACAGGAARCAT |
| | c.271A > G (p.Thr91Ala) | | | TTGTCTTTTGTTGCTGGAAAGC′] |
| 3452 | NM_001128227.2(GNE): | 10020 | GNE | [′CAGTGCAGAGCAGCACCTGATATCCRTGT |
| | c.604A > G (p.Met202Val) | | | GTGAGGACCATGATCGCATCCT′] |
| 3453 | NM_001029.3(RPS26): c.1A > G | 6231 | RPS26 | [′CCTCTCTCCGGTCCGTGCCTCCAAGDTGGT |
| | (p.Met1Val) | | | GAGTCTTCTTGCGTGGTGAGG′] |
| 3454 | NM_012082.3(ZFPM2): | −1 | — | [′TCAAACTAAGAAGCTCTCCACCTCCRGTAA |
| | c.1969A > G (p.Ser657Gly) | | | CAATGATGACAAAATTAATGG′] |
| 3455 | NM_012082.3(ZFPM2): c.89A > G | 23414 | ZFPM2 | [′GATGAGGAAGAAGAATGTCCATCAGRGGA |
| | (p.Glu30Gly) | | | AACAGACATCATCTCCAAAGGA′] |
| 3456 | NM_012082.3(ZFPM2): | −1 | — | [′AAAGTGTTTATCTCAGTCTGAGCGGRCGAC |
| | c.2527A > G (p.Thr843Ala) | | | CACGTCTCCCAAAAGGCTGCT′] |
| 3457 | NM_194248.2(OTOF): | 9381 | OTOF | [′TCTGGCTCCCCCTTCTCCTGCCTGCRGGTC |
| | c.766 − 2A > G | | | AGCATCACGGTGATCGAGGCC′] |
| 3458 | NM_003705.4(SLC25A12): c.1769 | 8604 | SLC25A12 | [′GCTCGAGTGTTTCGATCCTCTCCCCRGTTT |
| | A > G (p.Gln590Arg) | | | GGTGTTACCTTGGTCACTTAT′] |
| 3459 | NM_004621.5(TRPC6): c.428A > G | 7225 | TRPC6 | [′AATGCCCTACAGTTGGCAGTGGCCARTGA |
| | (p.Asn143Ser) | | | GCATCTGGAAATTACAGAACTT′] |
| 3460 | NM_152384.2(BBS5): | 129880 | BBS5 | [′TTTGGAATTTATCCAGTGATCAGGTRTTGT |
| | c.522 + 3A > G | | | GCAAAGAGCTAGTGAACCTTT′] |
| 3461 | NM_152384.2(BBS5): c.547A > G | 129880 | BBS5 | [′GGGCAATTTAGGAACCTTTTTTATTRCCAA |
| | (p.Thr183Ala) | | | TGTGAGAATTGTGTGGCATGC′] |
| 3462 | NM_001257342.1(BCS1L): c.232A | 617 | BCS1L | [′CCACAGTACCCGTACTCAGCACCTCRGTGT |
| | > G (p.Ser78Gly) | | | CGAGACTTCGTACCTTCAGCA′] |
| 3463 | NM_004328.4(BCS1L): c.148A > G | 617 | BCS1L | [′GGCATTCCGGCGCCATTACATGATCRCACT |
| | (p.Thr50Ala) | | | GGAAGTCCCTGCTCGAGACAG′] |
| 3464 | NM_003839.3(TNFRSF11A): c.508 | 8792 | TNFRSF11 | [′TGCCTTTTCCTCCACGGACAAATGCRGACC |
| | A > G (p.Arg170Gly) | | A | CTGGACCAAGTAAGTAACAAC′] |
| 3465 | NM_030761.4(WNT4): c.647A > G | 54361 | WNT4 | [′TGCCACGGGGTGTCAGGCTCCTGTGRGGT |
| | (p.Glu216Gly) | | | AAAGACGTGCTGGCGAGCCGTG′] |
| 3466 | NM_022817.2(PER2): c.1984A > G | 8864 | PER2 | [′GCTGGCACTGCCGGGCAAGGCAGAGRGTG |
| | (p.Ser662Gly) | | | TGGCGTCGCTCACCAGCCAGTG′] |
| 3467 | NM_002977.3(SCN9A): | −1 | — | [′TAGGGCACGACCAATCAAATACACARGAA |
| | c.1964A > G (p.Lys655Arg) | | | AAGGCGTTGTAGTTCCTATCTC′] |
| 3468 | NM_002977.3(SCN9A): c.184A > G | 6335 | SCN9A | [′GGAAGCTGGCAAACAGCTGCCCTTCRTCT |
| | (p.Ile62Val) | | | ATGGGGACATTCCTCCCGGCAT′] |
| 3469 | NM_002977.3(SCN9A): c.29A > G | 6335 | SCN9A | [′GCAATGTTGCCTCCCCCAGGACCTCRGAGC |
| | (p.Gln10Arg) | | | TTTGTCCATTTCACAAAACAG′] |
| 3470 | NM_001457.3(FLNB): c.604A > G | 2317 | FLNB | [′GCCTGTGGATAATGCACGAGAAGCCRTGC |
| | (p.Met202Val) | | | AGCAGGCAGATGACTGGCTGGG′] |
| 3471 | NM_003060.3(SLC22A5): | 6584 | SLC22A5 | [′GTAGGCATGGGCCAGATCTCCAACTRTGT |
| | c.632A > G (p.Tyr211Cys) | | | GGCAGCATTTGTCCTGGGTATG′] |
| 3472 | NM_000369.2(TSHR): c.1856A > G | 7253 | TSHR | [′CCGCAGTACAACCCAGGGGACAAAGRTAC |
| | (p.Asp619Gly) | | | CAAAATTGCCAAGAGGATGGCT′] |
| 3473 | NM_000369.2(TSHR): c.548A > G | 7253 | TSHR | [′TCTCTTCTCTCTGTTGGTTGTAGGARGCTG |
| | (p.Lys183Arg) | | | TACAACAATGGCTTTACTTCA′] |
| 3474 | NM_001430.4(EPAS1): | 2034 | EPAS1 | [′GGAGACACTGGCACCCTATATCCCCRTGG |
| | c.1603A > G (p.Met535Val) | | | ACGGGGAAGACTTCCAGCTAAG′] |
| 3475 | NM_024009.2(GJB3): c.421A > G | 2707 | GJB3 | [′GTTCAGCCTCATCTTCAAGCTCATCRTTGA |
| | (p.Ile141Val) | | | GTTCCTCTTCCTCTACCTGCT′] |
| 3476 | NM_024009.2(GJB3): c.497A > G | 2707 | GJB3 | [′ATGCCGCGCCTGGTGCAGTGTGCCADCGT |
| | (p.Asn166Ser) | | | GGCCCCCTGCCCCAACATCGTG′] |
| 3477 | NM_001080463.1(DYNC2H1): | 79659 | DYNC2H1 | [′TGTTTTTTTGCTTTTGTAGGTTGCCRTGGGT |
| | c.11284A > G (p.Met3762Val) | | | CAAGGTCAAGCTGATTTAGC′] |
| 3478 | NM_001080463.1(DYNC2H1): | 79659 | DYNC2H1 | [′CGCATGCCACCTGATGTAATTAGAGRTATT |
| | c.9044A > G (p.Asp3015Gly) | | | CTTGAAGGAGTTTTAAGGTTG′] |
| 3479 | NM_001080463.1(DYNC2H1): | 79659 | DYNC2H1 | [′GTTGACCCATCTCTGTTCCCTTCACRGGTA |
| | c.4610A > G (p.Gln1537Arg) | | | AGGGGGCTTACGTGTAGAAGC′] |
| 3480 | NM_001080463.1(DYNC2H1): | 79659 | DYNC2H1 | [′TGGTCCAAGTGGTGCTGGAAAATCARCGC |
| | c.5959A > G (p.Thr1987Ala) | | | TTTGGAGAATGTTAAGGGCTGC′] |
| 3481 | NM_007035.3(KERA): c.740A > G | 11081 | KERA | [′GTGGCCTTTTTGAGACTAAATCACARCAAA |
| | (p.Asn247Ser) | | | CTGTCAGATGAGGGTCTCCCA′] |
| 3482 | NM_003722.4(TP63): c.697A > G | 8626 | TP63 | [′TATCCGCGCCATGCCTGTCTACAAARAAGC |
| | (p.Lys233Glu) | | | TGAGCACGTCACGGAGGTGGT′] |
| 3483 | NM_003722.4(TP63): c.1052A > G | 8626 | TP63 | [′CGGATCTGTGCTTGCCCAGGAAGAGRCAG |
| | (p.Asp351Gly) | | | GAAGGCGGATGAAGATAGCATC′] |
| 3484 | NM_003722.4(TP63): c.1054A > G | 8626 | TP63 | [′GATCTGTGCTTGCCCAGGAAGAGACRGGA |
| | (p.Arg352Gly) | | | AGGCGGATGAAGATAGCATCAG′] |
| 3485 | NM_003742.2(ABCB11): | 8647 | ABCB11 | [′AGAACAGTGGCTGCTTTTGGTGGTGRGAA |
| | c.890A > G (p.Glu297Gly) | | | AAGAGAGGTTGAAAGGTTGGTT′] |
| 3486 | NM_006702.4(PNPLA6): | 10908 | PNPLA6 | [′GGACCTCACGTACCCAGTCACCTCCRTGTT |
| | c.3034A > G (p.Met1012Val) | | | CACTGGGTCTGCCTTTAACCG′] |
| 3487 | NM_006412.3(AGPAT2): | 10555 | AGPAT2 | [′GTACACCACGGGGACGATGGGCACCYGCA |
| | c.589 − 2A > G | | | GGCAGGGAGACGCACAGCTGAG′] |
| 3488 | NM_001084.4(PLOD3): c.668A > G | 8985 | PLOD3 | [′AAGTCTCGGATCTTTCAGAACCTCARCGGG |
| | (p.Asn223Ser) | | | GCTTTAGGTGAGGAGGAAGAC′] |
| 3489 | NM_138554.4(TLR4): c.896A > G | 7099 | TLR4 | [′GCATACTTAGACTACTACCTCGATGRTATT |
| | (p.Asp299Gly) | | | ATTGACTTATTTAATTGTTTG′] |
| 3490 | NM_001130978.1(DYSF): | 8291 | DYSF | [′GGGCCAGGTGCAGGAGACATCAAGGRTCC |
| | c.3892A > G (p.Ile1298Val) | | | TGGATGAGGTGAGCTGGGCGTG′] |
| 3491 | NM_001130978.1(DYSF): | 8291 | DYSF | [′CTTTTTTGTCTTCTCTCTGGGGCAGRGGCT |
| | c.5264A > G (p.Glu1755Gly) | | | GGCAGGATCCCAAACCCACAC′] |
| 3492 | NM_003494.3(DYSF): | 8291 | DYSF | [′CAGCTCTTAACCACTCCAGCCACTCRCTCT |
| | c.3443 − 33A > G | | | GGCACCTCTGTTTTTTCCCTT′] |
| 3493 | NM_003494.3(DYSF): | 8291 | DYSF | [′AACTTGTCCCCTCCCTGTGTCTTCTRGCTGT |
| | c.1285 − 2A > G | | | GCAGCAAGATCTTGGAGAAG′] |
| 3494 | NM_005450.4(NOG): c.665A > G | 9241 | NOG | [′CGCTGCGGCTGGATTCCCATCCAGTRCCCC |
| | (p.Tyr222Cys) | | | ATCATTTCCGAGTGCAAGTGC′] |
| 3495 | NM_004629.1(FANCG): | 2189 | FANCG | [′GATACAATTTTTTTCTTTCTCTTTTRGGCCT |
| | c.925 − 2A > G | | | TGAATGTCCCATGCAGTTCC′] |
| 3496 | NM_001701.3(BAAT): c.226A > G | 570 | BAAT | [′AGGGGATTATATGGGAGTCCACCCCRTGG |
| | (p.Met76Val) | | | GTCTCTTCTGGTCTCTGAAACC′] |
| 3497 | NM_006892.3(DNMT3B): | 1789 | DNMT3B | [′TTTGGCTTTCCTGTGCACTACACAGRCGTG |
| | c.2450A > G (p.Asp817Gly) | | | TCCAACATGGGCCGTGGTGCC′] |
| 3498 | NM_001360.2(DHCR7): c.839A > G | 1717 | DHCR7 | [′GCTGCTTCTGTCTTGCAGGCCATCTRCGTG |
| | (p.Tyr280Cys) | | | ATTGACTTCTTCTGGAACGAA′] |
| 3499 | NM_001360.2(DHCR7): c.1A > G | 1717 | DHCR7 | [′AAGGTTCTCTTTCTTGCAGGGCCCARTGGC |
| | (p.Met1Val) | | | TGCAAAATCGCAACCCAACAT′] |
| 3500 | NM_032119.3(ADGRV1): | 84059 | ADGRV1 | [′TATCATCAGAGCATGTCACAGATCTRTGGA |
| | c.18131A > G (p.Tyr6044Cys) | | | CTCATTCATGGTGACCTGTAA′] |
| 3501 | NM_004984.2(KIF5A): c.767A > G | 3798 | KIF5A | [′GTGCTGGACGAGGCAAAGAATATCARCAA |
| | (p.Asn256Ser) | | | GTCACTGTCAGCTCTGGGCAAT′] |
| 3502 | NM_004984.2(KIF5A): c.827A > G | 3798 | KIF5A | [′GTCTCCTTCCTCCCCCAGAAAAGCTRTGTT |
| | (p.Tyr276Cys) | | | CCATATCGTGACAGCAAAATG′] |
| 3503 | NM_007373.3(SHOC2): c.4A > G | 8036 | SHOC2 | [′TTTTGTCCAGGCTTGAGTCACCATGRGTAG |
| | (p.Ser2Gly) | | | TAGTTTAGGAAAAGAAAAAGA′] |
| 3504 | NM_016203.3(PRKAG2): | 51422 | PRKAG2 | [′TACTCCTTGATCAAAAATAAAATCCRCAG |
| | c.1148A > G (p.His383Arg) | | | ATTGCCCGTTATTGACCCTATC′] |
| 3505 | NM_016203.3(PRKAG2): | 51422 | PRKAG2 | [′GTCCTGCTTTTCTCTTTGCAGGTCCRTCGG |
| | c.1589A > G (p.His530Arg) | | | CTGGTGGTGGTAAATGAAGCA′] |
| 3506 | NM_003816.2(ADAM9): | 8754 | ADAM9 | [′TTAAAATTTGTATACGTGTAATGCARCATT |
| | c.411 − 8A > G | | | CAGAGGATTGCTGCATTTAGA′] |
| 3507 | NM_003002.3(SDHD): c.341A > G | 6392 | SDHD | [′GGCCTTGGACAAGTTGTTACTGACTRTGTT |
| | (p.Tyr114Cys) | | | CATGGGGATGCCTTGCAGAAA′] |
| 3508 | NM_003002.3(SDHD): c.149A > G | 6392 | SDHD | [′GAATGGTGTGGAGTGCAGCACATACRCTT |
| | (p.His50Arg) | | | GTCACCGAGCCACCATTGTATG′] |
| 3509 | NM_003002.3(SDHD): c.1A > G | 6392 | SDHD | [′ATGACCTTGAGCCCTCAGGAACGAGDTGG |
| | (p.Met1Val) | | | CGGTTCTCTGGAGGCTGAGTGC′] |
| 3510 | NM_002485.4(NBN): c.511A > G | 4683 | NBN | [′TCAGTAAAATATTCTGGCTTTACAAYTGGA |
| | (p.Ile171Val) | | | CGTCCACAAATGAGTGCACAT′] |
| 3511 | NM_170695.3(TGIF1): c.838A > G | 7050 | TGIF1 | [′TTCCTGTACAGCTGGGCCAAACCCARCCCT |
| | (p.Thr280Ala) | | | AGGGAGGCCACTGTCTCCTAA′] |
| 3512 | NM_002408.3(MGAT2): c.785A > G | 4247 | MGAT2 | [′CTTATACTTTTCCTAGAAGAGGATCRCTAC |
| | (p.His262Arg) | | | TTAGCCCCAGACTTTTACCAT′] |
| 3513 | NM_002408.3(MGAT2): c.952A > G | 4247 | MGAT2 | [′GAAAACTTGGAAATCCACAGAGCACRATA |
| | (p.Asn318Asp) | | | TGGGTCTAGCCTTGACCCGGAA′] |
| 3514 | NM_005422.2(TECTA): | 7007 | TECTA | [′CAGTCCAATGGCACGCATATCATGTRTAA |
| | c.5609A > G (p.Tyr1870Cys) | | | AAACACACTCTGGATCGAAAGC′] |
| 3515 | NM_012233.2(RAB3GAP1): | 22930 | RAB3GAP1 | [′GGTATTGTCTTTGCATGTATTTCCTRGGGA |
| | c.649 − 2A > G | | | TGTCCTTTAACTCCATTGCCT′] |
| 3516 | NM_005025.4(SERPINI1): | 5274 | SERPINI1 | [′GAGATTTTTCTTTCCAAAGCAATTCRCAAG |
| | c.1013A > G (p.His338Arg) | | | TCCTTCCTAGAGGTTAATGAA′] |
| 3517 | NM_001040667.2(HSF4): | 3299 | HSF4 | [′AGACGGTTTTCGGAAGGTGGTGAGCRTCG |
| | c.256A > G (p.Ile86Val) | | | AGCAGGGCGGCCTGCTTAGGCC′] |
| 3518 | NM_001008211.1(OPTN): | 10133 | OPTN | [′GTTTACTGTTCTGATTTTCATGCTGRAAGA |
| | c.1433A > G (p.Glu478Gly) | | | GCAGCGAGAGAGAAAATTCAT′] |
| 3519 | NM_000492.3(CFTR): c.2738A > G | 1080 | CFTR | [′GTGATTATCACCAGCACCAGTTCGTRTTAT |
| | (p.Tyr913Cys) | | | GTGTTTTACATTTACGTGGGA′] |
| 3520 | NM_000492.3(CFTR): | 1080 | CFTR | [′CTCAATAAGTCCTGGCCAGAGGGTGRGAT |
| | c.3717 + 4A > G | | | TTGAACACTGCTTGCTTTGTTA′] |
| 3521 | NM_000492.3(CFTR): c.1666A > G | 1080 | CFTR | [′ACTGAGTGGAGGTCAACGAGCAAGARTTT |
| | (p.Ile556Val) | | | CTTTAGCAAGGTGAATAACTAA′] |
| 3522 | NM_000492.3(CFTR): c.326A > G | 1080 | CFTR | [′TTACTGGGAAGAATCATAGCTTCCTRTGAC |
| | (p.Tyr109Cys) | | | CCGGATAACAAGGAGGAACGC′] |
| 3523 | NM_000492.3(CFTR): | 1080 | CFTR | [′TGGAATCTTTTTATATTTAGGGGTARGGAT |
| | c.273 + 4A > G | | | CTCATTTGTACATTCATTATG′] |
| 3524 | NM_000492.3(CFTR): c.3254A > G | 1080 | CFTR | [′CTGTTCCACAAAGCTCTGAATTTACRTACT |
| | (p.His1085Arg) | | | GCCAACTGGTTCTTGTACCTG′] |
| 3525 | NM_000492.3(CFTR): c.3700A > G | 1080 | CFTR | [′CATATTAGAGAACATTTCCTTCTCANTAAG |
| | (p.Ile1234Val) | | | TCCTGGCCAGAGGGTGAGATT′] |
| 3526 | NM_000492.3(CFTR): c.650A > G | 1080 | CFTR | [′GCACTCCTCATGGGGCTAATCTGGGRGTTG |
| | (p.Glu217Gly) | | | TTACAGGCGTCTGCCTTCTGT′] |
| 3527 | NM_001005360.2(DNM2): | 1785 | DNM2 | [′CCTCCACCCTCAGGAGAAAGAGAAGRAGT |
| | c.1684A > G (p.Lys562Glu) | | | ACATGCTGCCTCTGGACAACCT′] |
| 3528 | NM_001814.4(CTSC): c.857A > G | 1075 | CTSC | [′TCTCAGACCCCAATCCTAAGCCCTCRGGAG |
| | (p.Gln286Arg) | | | GTTGTGTCTTGTAGCCAGTAT′] |
| 3529 | NM_001814.4(CTSC): c.1235A > G | 1075 | CTSC | [′AATCATGCTGTTCTGCTTGTGGGCTRTGGC |
| | (p.Tyr412Cys) | | | ACTGACTCAGCCTCTGGGATG′] |
| 3530 | NM_001814.4(CTSC): c.1040A > G | 1075 | CTSC | [′CGTTATTACTCCTCTGAGTACCACTRTGTA |
| | (p.Tyr347Cys) | | | GGAGGTTTCTATGGAGGCTGC′] |
| 3531 | NM_005144.4(HR): | 55806 | HR | [′TCCGACCCCTCCAACCTGCGGCCCTRGAGC |
| | c.−218A > G | | | GCCCCCGCCGCCCCGGGGGAA′] |
| 3532 | NM_172107.2(KCNQ2): c.851A > G | 3785 | KCNQ2 | [′ACCACCATTGGCTACGGGGACAAGTRCCC |
| | (p.Tyr284Cys) | | | CCAGACCTGGAACGGCAGGCTC′] |
| 3533 | NM_001303.3(COX10): | 1352 | COX10 | [′GCCCTGAGCTGGGGCCTCCGTGAAGDCTA |
| | c.1007A > G (p.Asp336Gly) | | | CTCCCGGGGCGGCTACTGCATG′] |
| 3534 | NM_006214.3(PHYH): | 5264 | PHYH | [′AGAACGTTATTATCCAGAGTATACCYAAA |
| | c.135 − 2A > G | | | GGAGAAAAAGAATCCCAAAATA′] |
| 3535 | NM_002284.3(KRT86): c.340A > G | −1 | — | [′GGAGAAGGAGCAGATCAAGTCCCTCVACA |
| | (p.Asn114Asp) | | | GCAGGTTCGCGGCCTTCATCGA′] |
| 3536 | NM_003865.2(HESX1): c.541A > G | 8820 | HESX1 | [′TCTAATGGCGAAAAAAAATTTCAACRCAA |
| | (p.Thr181Ala) | | | ATCTGCTGGAATAGATAGAAAA′] |
| 3537 | NM_000303.2(PMM2): c.563A > G | 5373 | PMM2 | [′TTTGATGTCTTTCCTGATGGATGGGRCAAG |
| | (p.Asp188Gly) | | | AGATACTGTCTGCGACATGTG′] |
| 3538 | NM_000288.3(PEX7): | 5191 | PEX7 | [′TCTTTTGCTTTCTAAACACTTTTCARTGTTT |
| | c.340 − 10A > G | | | TTAGGTGTATAGTGTTGATT′] |
| 3539 | NM_004044.6(ATIC): c.1277A > G | 471 | ATIC | [′CTCATCGTAGCCACCATTGCTGTCARGTAC |
| | (p.Lys426Arg) | | | ACTCAGTCTAACTCTGTGTGC′] |
| 3540 | NM_000314.6(PTEN): c.368A > G | 5728 | PTEN | [′GATGACAATCATGTTGCAGCAATTCRCTGT |
| | (p.His123Arg) | | | AAAGCTGGAAAGGGACGAACT′] |
| 3541 | NM_000314.6(PTEN): c.278A > G | 5728 | PTEN | [′GTTGCACAATATCCTTTTGAAGACCRTAAC |
| | (p.His93Arg) | | | CCACCACAGCTAGAACTTATC′] |
| 3542 | NM_000314.6(PTEN): c.755A > G | 5728 | PTEN | [′CCTCAGCCGTTACCTGTGTGTGGTGRTATC |
| | (p.Asp252Gly) | | | AAAGTAGAGTTCTTCCACAAA′] |
| 3543 | NM_018488.2(TBX4): c.1592A > G | 9496 | TBX4 | [′TCCTTGTCCCGAGAATCTTCCTTACRGTAC |
| | (p.Gln531Arg) | | | CATTCAGGAATGGGGACTGTG′] |
| 3544 | NM_000223.3(KRT12): c.403A > G | 3859 | KRT12 | [′AGAAACTATGCAAAATCTTAATGATRGAT |
| | (p.Arg135Gly) | | | TAGCTTCCTACCTGGATAAGGT′] |
| 3545 | NM_000503.5(EYA1): c.1639A > G | 2138 | EYA1 | [′TGAGAGAATAATTCAAAGGTTTGGARGAA |
| | (p.Arg547Gly) | | | AAGTGGTGTATGTTGTTATAGG′] |
| 3546 | NM_000261.1(MYOC): c.1010A > G | 4653 | MYOC | [′GTGTACTCGGGGAGCCTCTATTTCCRGGGC |
| | (p.Gln337Arg) | | | GCTGAGTCCAGAACTGTCATA′] |
| 3547 | NM_000261.1(MYOC): c.1267A > G | 4653 | MYOC | [′ACAAACCTGGGAGACAAACATCCGTRAGC |
| | (p.Lys423Glu) | | | AGTCAGTCGCCAATGCCTTCAT′] |
| 3548 | NM_000474.3(TWIST1): | 7291 | TWIST1 | [′GACCCTCAAGCTGGCGGCCAGGTACRTCG |
| | c.466A > G (p.Ile156Val) | | | ACTTCCTCTACCAGGTCCTCCA′] |
| 3549 | NM_001089.2(ABCA3): | 21 | ABCA3 | [′ACAGATCACCGTCCTGCTGGGCCACRACG |
| | c.1702A > G (p.Asn568Asp) | | | GTGCCGGGAAGACCACCACCCT′] |
| 3550 | NM_005055.4(RAPSN): | 5913 | RAPSN | [′ATTCCTCAGAGGCCATGTGGCCCCARCTGG |
| | c.−210A > G | | | CAGCGACAGCTGCAGACGGGC′] |
| 3551 | NM_198217.2(ING1): c.515A > G | 3621 | ING1 | [′AACGAACCCACGTACTGTCTGTGCARCCA |
| | (p.Asn172Ser) | | | GGTCTCCTATGGGGAGATGATC′] |
| 3552 | NM_000430.3(PAFAH1B1): | 5048 | PAFAH1B1 | [′GATTTTGAACGAACTCTTAAAGGACRTAC |
| | c.446A > G (p.His149Arg) | | | AGACTCTGTACAGGACATTTCA′] |
| 3553 | NM_000579.3(CCR5): | −1 | — | [′TACGGGGAGAGTGGAGAAAAAGGGGRCA |
| | c.−301 + 246A > G | | | CAGGGTTAATGTGAAGTCCAGGA′] |
| 3554 | NM_000264.3(PTCH1): | −1 | — | [′ACTTTACGACCTACACAGGAGTTTCRGTAA |
| | c.2479A > G (p.Ser827Gly) | | | CGTGAAGTATGTCATGTTGGA′] |
| 3555 | NM_001184.3(ATR): c.2022A > G | 545 | ATR | [′TCTGCTGCTGCAATAAGATAAAAAAYCCA |
| | (p.Gly674=) | | | CTAACACAACTAGCCCGGATTA′] |
| 3556 | NM_005982.3(SIX1): c.386A > G | 6495 | SIX1 | [′ATCTGGGACGGCGAGGAGACCAGCTRCTG |
| | (p.Tyr129Cys) | | | CTTCAAGGAGAAGTCGAGGGGT′] |
| 3557 | NM_006267.4(RANBP2): | 5903 | RANBP2 | [′AGAAGACGCACACATAACTTTTGCTRTATT |
| | Gc.1966A > (p.Ile656Val) | | | GGATGCAGTAAATGGAAATAT′] |
| 3558 | NM_001001557.2(GDF6): | 392255 | GDF6 | [′CCGCCCAGCTGCTGCGTGCCCACCARATTG |
| | c.1271A > G (p.Lys424Arg) | | | ACTCCCATCAGCATTCTATAC′] |
| 3559 | NM_000557.4(GDF5): c.517A > G | 8200 | GDF5 | [′ACCCCCCATCACACCCCACGAGTACVTGCT |
| | (p.Met173Val) | | | CTCGCTGTACAGGACGCTGTC′] |
| 3560 | NM_000392.4(ABCC2): | 1244 | ABCC2 | [′CGAGAGAAGCTGACCATCATCCCCCRGGT |
| | c.4145A > G (p.Gln1382Arg) | | | GAGCTCTAGAACTTACTCGGGC′] |
| 3561 | NM_000396.3(CTSK): c.990A > G | 1513 | CTSK | [′ACCTGGCCAGCTTCCCCAAGATGTGRCTCC |
| | (p.Ter330Trp) | | | AGCCAGCCAAATCCATCCTGC′] |
| 3562 | NM_001127221.1(CACNA1A): | 773 | CACNA1A | [′AACGTCTTCAACATCCTCATCGTCTRCATG |
| | c.4151A > G (p.Tyr1384Cys) | | | CTATTCATGTTCATCTTCGCC′] |
| 3563 | NM_000178.2(GSS): c.656A > G | 2937 | GSS | [′CAAGAGAAGGAAAGAAACATATTTGRCCA |
| | (p.Asp219Gly) | | | GCGTGCCATAGAGAATGAGCTA′] |
| 3564 | NM_000901.4(NR3C2): | 4306 | NR3C2 | [′ACGCTCAACCGCTTAGCAGGCAAACRGAT |
| | c.2327A > G (p.Gln776Arg) | | | GATCCAAGTCGTGAAGTGGGCA′] |
| 3565 | NM_000901.4(NR3C2): | 4306 | NR3C2 | [′ATCAGCGACCAGCTGCCCAAGGTGGRGTC |
| | c.2915A > G (p.Glu972Gly) | | | GGGGAACGCCAAGCCGCTCTAC′] |
| 3566 | NM_004407.3(DMP1): c.1A > G | 1758 | DMP1 | [′CCAGGTAGAGGTATCACACCCAACTRTGA |
| | (p.Met1Val) | | | AGATCAGCATCCTGCTCATGTT′] |
| 3567 | NM_004999.3(MYO6): c.737A > G | 4646 | MYO6 | [′CAAGGCAAAGAGGAAAGAAATTATCRTAT |
| | (p.His246Arg) | | | CTTTTATAGGTTGTGTGCTGGT′] |
| 3568 | NM_000256.3(MYBPC3): | 4607 | MYBPC3 | [′CGCCAGCAACAAGTACGGCCTGGCCRCAG |
| | c.175A > G (p.Thr59Ala) | | | AGGGCACACGGCATACGCTGAC′] |
| 3569 | NM_000163.4(GHR): c.594A > G | 2690 | GHR | [′TGGAGTATGAACTTCAATACAAAGARGTA |
| | (p.Glu198=) | | | AATGAAACTAAATGGAAAATGG′] |
| 3570 | NM_000525.3(KCNJ11): | 3767 | KCNJ11 | [′ATCATGCTTGGCTGCATCTTCATGARGACT |
| | c.509A > G (p.Lys170Arg) | | | GCCCAAGCCCACCGCAGGGCT′] |
| 3571 | NM_000525.3(KCNJ11): | 3767 | KCNJ11 | [′CTGGTGGCCCCGCTGATCATCTACCRTGTC |
| | c.776A > G (p.His259Arg) | | | ATTGATGCCAACAGCCCACTC′] |
| 3572 | NM_000182.4(HADHA): | 3030 | HADHA | [′GAAAAAAACTGCAAATTAAATGAGAYACC |
| | c.180 + 3A > G | | | TTTGAATTGGGAGAGTTAATTC′] |
| 3573 | NM_005263.3(GFI1): c.1145A > G | 2672 | GFI1 | [′GGCAAGGCATTCAGCCAGAGCTCCARCCT |
| | (p.Asn382Ser) | | | CATCACCCACAGCCGCAAACAC′] |
| 3574 | NM_005263.3(GFI1): c.1208A > G | 2672 | GFI1 | [′CCCTTCGGCTGCGACCTCTGTGGGARGGGT |
| | (p.Lys403Arg) | | | TTCCAGAGGAAGGTGGACCTC′] |
| 3575 | NM_001893.4(CSNK1D): | 1453 | CSNK1D | [′TGCCATCAAGCTTGAATGTGTCAAARCCA |
| | c.130A > G (p.Thr44Ala) | | | AACACCCTCAGCTCCACATTGA′] |
| 3576 | NM_006204.3(PDE6C): | 5146 | PDE6C | [′AAACAGAAAGGACATTGCTCAGGAARTGC |
| | c.1363A > G (p.Met455Val) | | | TCATGAACCAAACCAAAGCCAC′] |
| 3577 | NM_006204.3(PDE6C): | 5146 | PDE6C | [′TGAAACAACCCATCCTTATTTCAACRGAAA |
| | c.1483 − 2A > G | | | GAGGACTTGCCAGACCCACGC′] |
| 3578 | NM_003476.4(CSRP3): c.206A > G | 8048 | CSRP3 | [′TGCTATGGGCGCAGATATGGCCCCARAGG |
| | (p.Lys69Arg) | | | GATCGGGTATGGACAAGGCGCT′] |
| 3579 | NM_005591.3(MRE11A): | 4361 | MRE11A | [′GTGAACTATCAAGATGGCAACCTCARCAT |
| | c.350A > G (p.Asn117Ser) | | | TTCAATTCCAGTGTTTAGTATT′] |
| 3580 | NM_000107.2(DDB2): c.730A > G | 1643 | DDB2 | [′TTGGAATCTCAGAATGCACAAAAAGRAAG |
| | (p.Lys244Glu) | | | TGACGCATGTGGCCCTGAACCC′] |
| 3581 | NM_001204.6(BMPR2): | 659 | BMPR2 | [′AAGGAGACAATCGAAGACTGTTGGGRCCA |
| | c.1454A > G (p.Asp485Gly) | | | GGATGCAGAGGCTCGGCTTACT′] |
| 3582 | NM_000336.2(SCNN1B): | 6338 | SCNN1B | [′ACAGAGAAGGCACTTCCTTCGGCCARCCC |
| | Gc.863A > (p.Asn288Ser) | | | TGGAACTGAATTCGGTGAGTTT′] |
| 3583 | NM_000447.2(PSEN2): c.715A > G | 5664 | PSEN2 | [′CTACCTCATCATGATCAGTGCGCTCRTGGC |
| | (p.Met239Val) | | | CCTAGTGTTCATCAAGTACCT′] |
| 3584 | NM_002181.3(IHH): c.284A > G | 3549 | IHH | [′CCAGACATCATCTTCAAGGACGAGGRGAA |
| | (p.Glu95Gly) | | | CACAGGCGCCGACCGCCTCATG′] |
| 3585 | NM_000310.3(PPT1): c.236A > G | 5538 | PPT1 | [′ACTTCTCTCTGGCTTCTTTTTTAGGRCGTGG |
| | (p.Asp79Gly) | | | AGAACAGCTTCTTCTTGAAT′] |
| 3586 | NM_003051.3(SLC16A1): | 6566 | SLC16A1 | [′GCGACCAATCGGGCCCAAGCCAACCRAGG |
| | c.610A > G (p.Lys204Glu) | | | CAGGGAAAGATAAGTCTAAAGC′] |
| 3587 | NM_005587.2(MEF2A): c.788A > G | 4205 | MEF2A | [′TCTCCCCCTCCACCAGGTGGTGGTARTCTT |
| | (p.Asn263Ser) | | | GGAATGAACAGTAGGAAACCA′] |
| 3588 | NM_000098.2(CPT2): c.638A > G | 1376 | CPT2 | [′TACCTGGTCAATGCGTATCCCCTGGRTATG |
| | (p.Asp213Gly) | | | TCCCAGTATTTTCGGCTTTTC′] |
| 3589 | NM_000098.2(CPT2): c.359A > G | 1376 | CPT2 | [′TTTTTAGGACCCTGGTTTGATATGTRCCTA |
| | (p.Tyr120Cys) | | | TCTGCTCGAGACTCCGTTGTT′] |
| 3590 | NM_001079668.2(NKX2-1): | 7080 | NKX2-1 | [′TCGCCGGGCCCATGAAGCGGGAGACHGTA |
| | c.464 − 2A > G | | | AGCGACAAACGCACAGCGTCGG′] |
| 3591 | NM_004387.3(NKX2-5): | 1482 | NKX2-5 | [′AACTTCGTGAACTTCGGCGTCGGGGRCTTG |
| | c.896A > G (p.Asp299Gly) | | | AATGCGGTTCAGAGCCCCGGG′] |
| 3592 | NM_004387.3(NKX2-5): | 1482 | NKX2-5 | [′GCTGAAACTCACGTCCACGCAGGTCRAGA |
| | c.547A > G (p.Lys183Glu) | | | TCTGGTTCCAGAACCGGCGCTA′] |
| 3593 | NM_178138.4(LHX3): c.332A > G | 8022 | LHX3 | [′GTGCGCCGCGCCCAGGACTTCGTGTRCCAC |
| | (p.Tyr111Cys) | | | CTGCACTGCTTTGCCTGCGTC′] |
| 3594 | NM_001698.2(AUH): | 549 | AUH | [′CTTGTACTTTTTTTCCCCTTTAACTRGGAAT |
| | c.263 − 2A > G | | | TGTGGTGCTTGGAATAAACA′] |
| 3595 | NM_001698.2(AUH): | 549 | AUH | [′ACATATTTAATATTTTGTTTTTCTTRGACCA |
| | c.943 − 2A > G | | | TTCCAACAAAAGACAGACTT′] |
| 3596 | NM_001876.3(CPT1A): | 1374 | CPT1A | [′ATGTGTGTTTCACGTAGGTGGTTTGRCAAG |
| | c.1361A > G (p.Asp454Gly) | | | TCGTTCACGTTTGTTGTCTTC′] |
| 3597 | NM_001876.3(CPT1A): | 1374 | CPT1A | [′CGGCTGCTGAAGCCCCGGGAGATGGRGCA |
| | c.1079A > G (p.Glu360Gly) | | | GCAGATGCAGAGGATCCTGGAC′] |
| 3598 | NM_001876.3(CPT1A): | 1374 | CPT1A | [′TCCATTGACAGCCTCCAGCTGGGCTRTGCG |
| | c.1493A > G (p.Tyr498Cys) | | | GAGGATGGGCACTGCAAAGGC′] |
| 3599 | NM_000352.4(ABCC8): | 6833 | ABCC8 | [′CACCCTGCGCTCACGCCTCTCCATCRTCCT |
| | c.4270A > G (p.Ile1424Val) | | | GCAGGACCCCGTCCTCTTCAG′] |
| 3600 | NM_000352.4(ABCC8): c.215A > G | 6833 | ABCC8 | [′ACATGGCTTCATTTCCCTGGGCACARCCTG |
| | (p.Asn72Ser) | | | CGGTGGATCCTGACCTTCATG′] |
| 3601 | NM_002180.2(IGHMBP2): | 3508 | IGHMBP2 | [′TCTCAGAAAGAACTTGCCATCATCCRTGGA |
| | c.638A > G (p.His213Arg) | | | CCTCCTGGCACTGGGAAAACC′] |
| 3602 | NM_033163.3(FGF8): c.298A > G | 2253 | FGF8 | [′GAAGCACGTGCAGGTCCTGGCCAACRAGC |
| | (p.Lys100Glu) | | | GCATCAACGCCATGGCAGAGGA′] |
| 3603 | NM_006180.4(NTRK2): | 4915 | NTRK2 | [′TCCCGGGACGTGTACAGCACTGACTRCTAC |
| | c.2165A > G (p.Tyr722Cys) | | | AGGGTGAGTAGCTGTGCAGAT′] |
| 3604 | NM_033028.4(BBS4): | 585 | BBS4 | [′TCAGAAGCATTTTTCTCCCTCTTTCRGGCT |
| | c.157 − 2A > G | | | GTTATCAAAGAACAGCTTCAA′] |
| 3605 | NM_000409.3(GUCA1A): | 2978 | GUCA1A | [′AAGCTCCGCTGGTACTTCAAGCTCTRTGAT |
| | c.296A > G (p.Tyr99Cys) | | | GTAGATGGCAACGGCTGCATT′] |
| 3606 | NM_000344.3(SMN1): c.815A > G | 6606 | SMN1 | [′TTAATTTCATGGTACATGAGTGGCTRTCAT |
| | (p.Tyr272Cys) | | | ACTGGCTATTATATGGTAAGT′] |
| 3607 | NM_000344.3(SMN1): c.784A > G | 6606 | SMN1 | [′TCTTGATGATGCTGATGCTTTGGGARGTAT |
| | (p.Ser262Gly) | | | GTTAATTTCATGGTACATGAG′] |
| 3608 | NM_000095.2(COMP): c.1358A > G | 1311 | COMP | [′CGGGACAACTGTCCCACGGTGCCTARCAG |
| | (p.Asn453Ser) | | | TGCCCAGGAGGACTCAGACCAC′] |
| 3609 | NM_000095.2(COMP): c.1418A > G | 1311 | COMP | [′GGTGATGCCTGCGACGACGACGACGRCAA |
| | (p.Asp473Gly) | | | TGACGGAGTCCCTGACAGTCGG′] |
| 3610 | NM_002047.2(GARS): c.374A > G | 2617 | GARS | [′GATATTGTAGACCGAGCAAAAATGGRAGA |
| | (p.Glu125Gly) | | | TACCCTGAAGAGGAGGTTTTTC′] |
| 3611 | NM_001166107.1(HMGCS2): | 3158 | HMGCS2 | [′GGCATAGATACCACCAATGCCTGCTRCGG |
| | c.500A > G (p.Tyr167Cys) | | | TGGTACTGCCTCCCTCTTCAAT′] |
| 3612 | NM_000161.2(GCH1): c.671A > G | 2643 | GCH1 | [′CGAGGTGTACAGAAAATGAACAGCARAAC |
| | (p.Lys224Arg) | | | TGTGACCAGCACAATGTTGGGT′] |
| 3613 | NM_001024630.3(RUNX2): | 860 | RUNX2 | [′ATCTGCAGGCAAGAGTTTCACCTTGRCCAT |
| | c.598A > G (p.Thr200Ala) | | | AACCGTCTTCACAAATCCTCC′] |
| 3614 | NM_000423.2(KRT2): c.556A > G | 3849 | KRT2 | [′AGAGCGTGAGCAGATCAAAACTCTCDACA |
| | (p.Asn186Asp) | | | ACAAATTTGCCTCCTTCATTGA′] |
| 3615 | NM_000215.3(JAK3): c.299A > G | 3718 | JAK3 | [′GATGCCAGCACCCAAGTCCTGCTGTRCAG |
| | (p.Tyr100Cys) | | | GATTCGGTAGGAAGTGCCCCCC′] |
| 3616 | NM_000335.4(SCN5A): | 6331 | SCN5A | [′CTCTGTCCACTTGAGGTGGTGGTCARTGCC |
| | c.3971A > G (p.Asn1324Ser) | | | CTGGTGGGCGCCATCCCGTCC′] |
| 3617 | NM_000335.4(SCN5A) | 6331 | SCN5A | [′AGTGAGGACGACTTCGATATGTTCTRTGAG |
| | c.5381A > G (p.Tyr1794Cys) | | | ATCTGGGAGAAATTTGACCCA′] |
| 3618 | NM_001014797.2(KCNMA1): | 3778 | KCNMA1 | [′AAGGACTTTCTGCACAAGGACCGGGRTGA |
| | c.1301A > G (p.Asp434Gly) | | | CGTCAATGTGGAGATCGTTTTT′] |
| 3619 | NM_004380.2(CREBBP): | 1387 | CREBBP | [′TATAATCGCAAGACATCCCGAGTCTRTAA |
| | c.3524A > G (p.Tyr1175Cys) | | | GTTTTGCAGTAAGCTTGCAGAG′] |
| 3620 | NM_000023.2(SGCA): c.410A > G | 6442 | SGCA | [′GGCCCCCTGCTGCCATACCAAGCCGRGTTC |
| | (p.Glu137Gly) | | | CTGGTGCGCAGCCACGATGCG′] |
| 3621 | NM_002835.3(PTPN12): | 5782 | PTPN12 | [′GGAGAAAAAGAAGAAAATGTTAAAARGA |
| | c.182A > G (p.Lys61Arg) | | | ACAGATACAAGGACATACTGCCA′] |
| 3622 | NM_000211.4(ITGB2): | 3689 | ITGB2 | [′GGGGAGCTGTCTGAGGACTCCAGCARTGT |
| | c.1052A > G (p.Asn351Ser) | | | GGTCCAACTCATTAAGAATGCT′] |
| 3623 | NM_005502.3(ABCA1): | 19 | ABCA1 | [′GTCTGGGGGGGCTTCGCCTACTTGCRGGAT |
| | c.1790A > G (p.Gln597Arg) | | | GTGGTGGAGCAGGCAATCATC′] |
| 3624 | NM_005502.3(ABCA1): | 19 | ABCA1 | [′CAGATCACCTCCTTCCTGGGCCACARTGGA |
| | c.2804A > G (p.Asn935Ser) | | | GCGGGGAAGACGACCACCATG′] |
| 3625 | m.5843A > G | 4579 | MT-TY | [′CTCGGAGCTGGTAAAAAGAGGCCTARCCC |
| | | | | CTGTCTTTAGATTTACAGTCCA′] |
| 3626 | m.7445A > G | −1 | — | [′AAGAACCCGTATACATAAAATCTAGVCAA |
| | | | | AAAAGGAAGGAATCGAACCCCC′] |
| 3627 | m.8344A > G | 4566 | MT-TK | [′ACCTTTTAAGTTAAAGATTAAGAGARCCA |
| | | | | ACACCTCTTTACAGTGAAATGC′] |
| 3628 | m.8296A > G | 4566 | MT-TK | [′ACCCCCTCTACCCCCTCTAGAGCCCRCTGT |
| | | | | AAAGCTAACTTAGCATTAACC′] |
| 3629 | m.12320A > G | 4568 | MT-TL2 | [′TCTTAGGCCCCAAAAATTTTGGTGCRACTC |
| | | | | CAAATAAAAGTAATAACCATG′] |
| 3630 | m.3243A > G | 4567 | MT-TL1 | [′AGAACAGGGTTTGTTAAGATGGCAGRGCC |
| | | | | CGGTAATCGCATAAAACTTAAA′] |
| 3631 | m.3252A > G | 4567 | MT-TL1 | [′TTTGTTAAGATGGCAGAGCCCGGTARTCGC |
| | | | | ATAAAACTTAAAACTTTACAG′] |
| 3632 | m.3251A > G | 4567 | MT-TL1 | [′GTTTGTTAAGATGGCAGAGCCCGGTRATC |
| | | | | GCATAAAACTTAAAACTTTACA′] |
| 3633 | m.3260A > G | 4567 | MT-TL1 | [′GATGGCAGAGCCCGGTAATCGCATARAAC |
| | | | | TTAAAACTTTACAGTCAGAGGT′] |
| 3634 | m.3274A > G | 4567 | MT-TL1 | [′GTAATCGCATAAAACTTAAAACTTTRCAGT |
| | | | | CAGAGGTTCAATTCCTCTTCT′] |
| 3635 | m.4317A > G | 4565 | MT-TI | [′GATAGAGTAAATAATAGGAGCTTAARCCC |
| | | | | CCTTATTTCTAGGACTATGAGA′] |
| 3636 | m.4269A > G | 4565 | MT-TI | [′GCATTCCCCCTCAAACCTAAGAAATRTGTC |
| | | | | TGATAAAAGAGTTACTTTGAT′] |
| 3637 | m.4295A > G | 4565 | MT-TI | [′TGTCTGATAAAAGAGTTACTTTGATRGAGT |
| | | | | AAATAATAGGAGCTTAAACCC′] |
| 3638 | m.4300A > G | 4565 | MT-TI | [′GATAAAAGAGTTACTTTGATAGAGTRAAT |
| | | | | AATAGGAGCTTAAACCCCCTTA′] |
| 3639 | m.10044A > G | 4563 | MT-TG | [′CTTCCAATTAACTAGTTTTGACAACRTTCA |
| | | | | AAAAAGAGTAATAAACTTCGC′] |
| 3640 | m.7526A > G | 4555 | MT-TD | [′TCCATGACTTTTTCAAAAAGGTATTRGAAA |
| | | | | AACCATTTCATAACTTTGTCA′] |
| 3641 | m.10438A > G | 4573 | MT-TR | [′AGTTTAAACAAAACGAATGATTTCGRCTC |
| | | | | ATTAAATTATGATAATCATATT′] |
| 3642 | m.1555A > G | 4549 | MT-RNR1 | [′ACCCCTACGCATTTATATAGAGGAGRCAA |
| | | | | GTCGTAACATGGTAAGTGTACT′] |
| 3643 | m.827A > G | 4549 | MT-RNR1 | [′CACCCCCACGGGAAACAGCAGTGATNARC |
| | | | | CTTTAGCAATAAACGAAAGTTT′] |
| 3644 | m.15579A > G | 4519 | MT-CYB | [′CCCGAATGATATTTCCTATTCGCCTRCACA |
| | | | | ATTCTCCGATCCGTCCCTAAC′] |
| 3645 | m.14495A > G | 4541 | MT-ND6 | [′TCCAAAGACAACCATCATTCCCCCTRAATA |
| | | | | AATTAAAAAAACTATTAAACC′] |
| 3646 | m.12770A > G | 4540 | MT-ND5 | [′CTATTCCAACTGTTCATCGGCTGAGRGGGC |
| | | | | GTAGGAATTATATCCTTCTTG′] |
| 3647 | m.11084A > G | 4538 | MT-ND4 | [′CTCCCTACAAATCTCCTTAATTATARCATT |
| | | | | CACAGCCACAGAACTAATCAT′] |
| 3648 | m.3397A > G | 4535 | MT-ND1 | [′TACCGAACGAAAAATTCTAGGCTATRTAC |
| | | | | AACTACGCAAAGGCCCCAACGT′] |
| 3649 | m.4136A > G | 4535 | MT-ND1 | [′CTGTTCTTATGAATTCGAACAGCATRCCCC |
| | | | | CGATTCCGCTACGACCAACTC′] |
| 3650 | m.3796A > G | 4535 | MT-ND1 | [′CCGTTTACTCAATCCTCTGATCAGGNTGAG |
| - | | | | CATCAAACTCAAACTACGCCC′, |
| 3654 | | | | ′AATAAGTGGCTCCTTTAACCTCTCCDCCCTT |
| | | | | ATCACAACACAAGAACACCT′, |
| | | | | ′ACCCCCTTGACCTTGCCGAAGGGGANTCCG |
| | | | | AACTAGTCTCAGGCTTCAACA′, |
| | | | | ′CGAATACGCCGCAGGCCCCTTCGCCNTATT |
| | | | | CTTCATAGCCGAATACACAAA′, |
| | | | | ′ATTTTGTCACCAAGACCCTACTTCTNACCTC |
| | | | | CCTGTTCTTATGAATTCGAA′] |
| 3655 | NM_003334.3(UBA1): c.1639A > G | 7317 | UBA1 | [′AATGAATCCACATATCCGGGTGACARGCC |
| | (p.Ser547Gly) | | | ACCAGAACCGTGTGGGTCCTGA′] |
| 3656 | NM_000354.5(SERPINA7): | 6906 | SERPINA7 | [′GATCAAAAGGATTTGCCCACTGGGCYTAA |
| | c.623 − 2A > G | | | AATACAAAGAAAAGAGAGGTGT′] |
| 3657 | NM_000044.3(AR): c.2291A > G | 367 | AR | [′ACCAATGTCAACTCCAGGATGCTCTRCTTC |
| | (p.Tyr764Cys) | | | GCCCCTGATCTGGTTTTCAAT′] |
| 3658 | NM_000044.3(AR): c.2362A > G | 367 | AR | [′CCGGATGTACAGCCAGTGTGTCCGARTGA |
| | (p.Met788Val) | | | GGCACCTCTCTCAAGAGTTTGG′] |
| 3659 | NM_000044.3(AR): c.2632A > G | 367 | AR | [′GATTGCGAGAGAGCTGCATCAGTTCRCTTT |
| | (p.Thr878Ala) | | | TGACCTGCTAATCAAGTCACA′] |
| 3660 | NM_000044.3(AR): c.2708A > G | 367 | AR | [′ATGATGGCAGAGATCATCTCTGTGCRAGT |
| | (p.Gln903Arg) | | | GCCCAAGATCCTTTCTGGGAAA′] |
| 3661 | NM_002764.3(PRPS1): c.341A > G | 5631 | PRPS1 | [′CCAATCTCAGCCAAGCTTGTTGCAARTATG |
| | (p.Asn114Ser) | | | CTATCTGTAGCAGGTGCAGAT′] |
| 3662 | NM_000194.2(HPRT1): c.602A > G | 3251 | HPRT1 | [′CTTGACTATAATGAATACTTCAGGGRTTTG |
| | (p.Asp201Gly) | | | AATGTAAGTAATTGCTTCTTT′] |
| 3663 | NM_000194.2(HPRT1): c.155A > G | 3251 | HPRT1 | [′TGTAGGACTGAACGTCTTGCTCGAGRTGTG |
| | (p.Asp52Gly) | | | ATGAAGGAGATGGGAGGCCAT′] |
| 3664 | NM_000132.3(F8): c.872A > G | 2157 | F8 | [′CCTGAAGTGCACTCAATATTCCTCGRAGGT |
| | (p.Glu291Gly) | | | CACACATTTCTTGTGAGGAAC′] |
| 3665 | NM_000132.3(F8): c.5183A > G | 2157 | F8 | [′GCTGCAGTGGAGAGGCTCTGGGATTRTGG |
| | (p.Tyr1728Cys) | | | GATGAGTAGCTCCCCACATGTT′] |
| 3666 | NM_000132.3(F8): c.5821A > G | 2157 | F8 | [′TTCTCTGTGTCCTTCTCCAGCAATCRATGG |
| | (p.Asn1941Asp) | | | CTACATAATGGATACACTACC′] |
| 3667 | NM_000132.3(F8): c.328A > G | 2157 | F8 | [′TACAGTGGTCATTACACTTAAGAACRTGGC |
| | (p.Met110Val) | | | TTCCCATCCTGTCAGTCTTCA′] |
| 3668 | NM_000132.3(F8): c.398A > G | 2157 | F8 | [′TTTCTTCCTGCTATAGGAGCTGAATRTGAT |
| | (p.Tyr133Cys) | | | GATCAGACCAGTCAAAGGGAG′] |
| 3669 | NM_000132.3(F8): c.404A > G | 2157 | F8 | [′CCTGCTATAGGAGCTGAATATGATGRTCA |
| | (p.Asp135Gly) | | | GACCAGTCAAAGGGAGAAAGAA′] |
| 3670 | NM_000132.3(F8): c.940A > G | 2157 | F8 | [′GGAAATCTCGCCAATAACTTTCCTTRCTGC |
| | (p.Thr314Ala) | | | TCAAACACTCTTGATGGACCT′] |
| 3671 | NM_000132.3(F8): c.1226A > G | 2157 | F8 | [′TGGGTACATTACATTGCTGCTGAAGRGGA |
| | (p.Glu409Gly) | | | GGACTGGGACTATGCTCCCTTA′] |
| 3672 | NM_000132.3(F8): c.1331A > G | 2157 | F8 | [′CAGCGGATTGGTAGGAAGTACAAAARAGT |
| | (p.Lys444Arg) | | | CCGATTTATGGCATACACAGAT′] |
| 3673 | NM_000132.3(F8): c.1418A > G | 2157 | F8 | [′TCAGGAATCTTGGGACCTTTACTTTRTGGG |
| | (p.Tyr473Cys) | | | GAAGTTGGAGACACACTGTTG′] |
| 3674 | NM_000132.3(F8): c.1660A > G | 2157 | F8 | [′TCGGTGCCTGACCCGCTATTACTCTRGTTT |
| | (p.Ser554Gly) | | | CGTTAATATGGAGAGAGATCT′] |
| 3675 | NM_000132.3(F8): c.1682A > G | 2157 | F8 | [′TCTAGTTTCGTTAATATGGAGAGAGRTCTA |
| | (p.Asp561Gly) | | | GCTTCAGGACTCATTGGCCCT′] |
| 3676 | NM_000132.3(F8): c.1892A > G | 2157 | F8 | [′GAGGATCCAGAGTTCCAAGCCTCCARCAT |
| | (p.Asn631Ser) | | | CATGCACAGTGAGTAAAGCAGC′] |
| 3677 | NM_000132.3(F8): c.5600A > G | 2157 | F8 | [′TTCCCTCCCTAGGAAAAAGATGTGCRCTCA |
| | (p.His1867Arg) | | | GGCCTGATTGGACCCCTTCTG′] |
| 3678 | NM_000132.3(F8): c.5822A > G | 2157 | F8 | [′TCTCTGTGTCCTTCTCCAGCAATCARTGGC |
| | (p.Asn1941Ser) | | | TACATAATGGATACACTACCT′] |
| 3679 | NM_000132.3(F8): c.6113A > G | 2157 | F8 | [′AGCACACTTTTTCTGGTGTACAGCARTAGT |
| | (p.Asn2038Ser) | | | GAGTAGCAATGTGGGCAGAGG′] |
| 3680 | NM_000132.3(F8): c.6278A > G | 2157 | F8 | [′AAGTGTTATTTTAATTGGTAGGTGGRTCTG |
| | (p.Asp2093Gly) | | | TTGGCACCAATGATTATTCAC′] |
| 3681 | NM_000132.3(F8): c.6371A > G | 2157 | F8 | [′TACATCTCTCAGTTTATCATCATGTRTAGT |
| | | | | CTTGATGGGAAGAAGTGGCAG′] |
| 3682 | NM_000132.3(F8): c.6794A > G | 2157 | F8 | [′ATGAAAGTCACAGGAGTAACTACTCRGGG |
| | (p.Gln2265Arg) | | | AGTAAAATCTCTGCTTACCAGC′] |
| 3683 | NM_000132.3(F8): c.104A > G | 2157 | F8 | [′GGTGCAGTGGAACTGTCATGGGACTRTAT |
| | (p.Tyr35Cys) | | | GCAAAGTGATCTCGGTGAGCTG′] |
| 3684 | NM_000402.4(G6PD): c.466A > G | 2539 | G6PD | [′CAACTCCTATGTGGCTGGCCAGTACRATGA |
| | (p.Asn156Asp) | | | TGCAGCCTCCTACCAGCGCCT′] |
| 3685 | NM_000402.4(G6PD): c.583A > G | 2539 | G6PD | [′GCTCCGGGCTCCCAGCAGAGGCTGGRACC |
| | (p.Asn195Asp) | | | GCATCATCGTGGAGAAGCCCTT′] |
| 3686 | NM_002049.3(GATA1): c.653A > G | 2623 | GATA1 | [′ACAGCCACTCCACTGTGGCGGAGGGRCAG |
| | (p.Asp218Gly) | | | GACAGGCCACTACCTATGCAAC′] |
| 3687 | NM_001097642.2(GJB1): | 2705 | GJB1 | [′CAGCCTGGCTGCAACAGCGTTTGCTRTGAC |
| | c.194A > G (p.Tyr65Cys) | | | CAATTCTTCCCCATCTCCCAT′] |
| 3688 | NM_000166.5(GJB1): c.614A > G | 2705 | GJB1 | [′GCCTCTGGCATCTGCATCATCCTCARTGTG |
| | (p.Asn205Ser) | | | GCCGAGGTGGTGTACCTCATC′] |
| 3689 | NM_000032.4(ALAS2): | 212 | ALAS2 | [′CCGTCCTGTACACTTTGAGCTCATGRGTGA |
| | c.1702A > G (p.Ser568Gly) | | | GTGGGAACGTTCCTACTTCGG′] |
| 3690 | NM_000202.6(IDS): c.404A > G | 3423 | IDS | [′GGCTATGTGACCATGTCGGTGGGAARAGT |
| | (p.Lys135Arg) | | | CTTTCACCCTGGTACTGCTCCA′] |
| 3691 | NM_000292.2(PHKA2): c.896A > G | 5256 | PHKA2 | [′TATGGATGCTGTCGCTTCCTTCGAGRTGGT |
| | (p.Asp299Gly) | | | TATAAAACTCCAAGAGAGGTT′] |
| 3692 | NM_000292.2(PHKA2): c.565A > G | 5256 | PHKA2 | [′TTATGGAATGTGGGAGCGTGGAGATRAGA |
| | (p.Lys189Glu) | | | CTAATCAGGGCATCCCGGAATT′] |
| 3693 | NM_000351.4(STS): c.1331A > G | 412 | STS | [′CGCTCCGATCATGAGTTTCTCTTCCRTTAC |
| | (p.His444Arg) | | | TGCAACGCCTACTTAAATGCT′] |
| 3694 | NM_000133.3(F9): c.278A > G | 2158 | F9 | [′TTCAATTTCTTAACCTATCTCAAAGRTGGA |
| | (p.Asp93Gly) | | | GATCAGTGTGAGTCCAATCCA′] |
| 3695 | NM_000133.3(F9): c.329A > G | 2158 | F9 | [′TGTTTAAATGGCGGCAGTTGCAAGGRTGA |
| | (p.Asp110Gly) | | | CATTAATTCCTATGAATGTTGG′] |
| 3696 | NM_000133.3(F9): c.917A > G | 2158 | F9 | [′CGAATTATTCCTCACCACAACTACARTGCA |
| | (p.Asn306Ser) | | | GCTATTAATAAGTACAACCAT′] |
| 3697 | NM_000133.3(F9): c.1180A > G | 2158 | F9 | [′TACAAAGTTCACCATCTATAACAACRTGTT |
| | (p.Met394Val) | | | CTGTGCTGGCTTCCATGAAGG′] |
| 3698 | NM_000133.3(F9): c.1231A > G | 2158 | F9 | [′AGGTAGAGATTCATGTCAAGGAGATRGTG |
| | (p.Ser411Gly) | | | GGGGACCCCATGTTACTGAAGT′] |
| 3699 | NM_000266.3(NDP): c.131A > G | 4693 | NDP | [′CCTCGACGCTGCATGAGGCACCACTRTGTG |
| | (p.Tyr44Cys) | | | GATTCTATCAGTCACCCATTG′] |
| 3700 | NM_000266.3(NDP): c.1A > G | 4693 | NDP | [′TCTAGAGAAGTTTTTCCTTACAACARTGAG |
| | (p.Met1Val) | | | AAAACATGTACTAGCTGCATC′] |
| 3701 | NM_000266.3(NDP): c.125A > G | 4693 | NDP | [′TCGGACCCTCGACGCTGCATGAGGCRCCA |
| | (p.His42Arg) | | | CTATGTGGATTCTATCAGTCAC′] |
| 3702 | NM_000169.2(GLA): c.886A > G | −1 | — | [′GGCTATCATGGCTGCTCCTTTATTCRTGTC |
| | (p.Met296Val) | | | TAATGACCTCCGACACATCAG′] |
| 3703 | NM_000169.2(GLA): c.101A > G | −1 | — | [′ATCCCTGGGGCTAGAGCACTGGACARTGG |
| | (p.Asn34Ser) | | | ATTGGCAAGGACGCCTACCATG′] |
| 3704 | NM_000169.2(GLA): c.644A > G | −1 | — | [′GAGTCATATCTGTTTTCACAGCCCARTTAT |
| | (p.Asn215Ser) | | | ACAGAAATCCGACAGTACTGC′] |
| 3705 | NM_000169.2(GLA): c.1228A > G | −1 | — | [′AAGGTTAAGAAGTCACATAAATCCCRCAG |
| | (p.Thr410Ala) | | | GCACTGTTTTGCTTCAGCTAGA′] |
| 3706 | NM_000169.2(GLA): c.815A > G | −1 | — | [′TCTTTTTCTCAGTTAGTGATTGGCARCTTTG |
| | (p.Asn272Ser) | | | GCCTCAGCTGGAATCAGCAA′] |
| 3707 | NM_153252.4(BRWD3): | 254065 | BRWD3 | [′AGAAGATAAAGAGAAAAAAGAAACARAA |
| | c.4786A > G (p.Lys1596Glu) | | | GAGAAATCTCATTTATCCACCTC′] |
| 3708 | NM_004463.2(FGD1): c.1396A > G | 2245 | FGD1 | [′GCAGAAACTGGCCCCCTTCCTCAAGRTGTA |
| | (p.Met466Val) | | | TGGTGAGTATGTGAAGAACTT′] |
| 3709 | NM_000054.4(AVPR2): c.614A > G | 554 | AVPR2 | [′GCGGAGCCCTGGGGCCGTCGCACCTRTGT |
| | (p.Tyr205Cys) | | | CACCTGGATTGCCCTGATGGTG′] |
| 3710 | NM_000054.4(AVPR2): c.839A > G | 554 | AVPR2 | [′ATGACGCTAGTGATTGTGGTCGTCTRTGTG |
| | (p.Tyr280Cys) | | | CTGTGCTGGGCACCCTTCTTC′] |
| 3711 | NM_000276.3(OCRL): c.1436A > G | 4952 | OCRL | [′ATCAAGTTCATCCCCACTTATAAGTRTGAC |
| | (p.Tyr479Cys) | | | TCTAAAACAGACCGGTGGGAT′] |
| 3712 | NM_000397.3(CYBB): c.302A > G | 1536 | CYBB | [′CAACTGGACAGGAATCTCACCTTTCRTAAA |
| | (p.His101Arg) | | | ATGGTGGCATGGATGATTGCA′] |
| 3713 | NM_000397.3(CYBB): c.1499A > G | 1536 | CYBB | [′GCTGTGCACCATGATGAGGAGAAAGRTGT |
| | (p.Asp500Gly) | | | GATCACAGGCCTGAAACAAAAG′] |
| 3714 | NM_001205019.1(GK): c.880A > G | 2710 | GK | [′TGGAACAGGATGTTTCTTACTATGTRATAC |
| | (p.Asn294Asp) | | | AGGCCATAAGGTTGGTTTTTT′] |
| 3715 | NM_005710.2(PQBP1): c.194A > G | 10084 | PQBP1 | [′CGGCCCCACAGCGGGCTCCCTTACTRCTGG |
| | (p.Tyr65Cys) | | | AATGCAGACACAGACCTTGTA′] |
| 3716 | NM_198270.3(NHS): | 4810 | NHS | [′CTGAACCTGATTGTACTTTGTTTGCRGTCC |
| | c.853 − 2A > G | | | CATCCCCCAGAGGATGAAGAT′] |
| 3717 | NM_000252.2(MTM1): c.566A > G | 4534 | MTM1 | [′CACCATTGGAGAATAACTTTTATTARTAAG |
| | (p.Asn189Ser) | | | TGCTATGAGCTCTGTGACACT′] |
| 3718 | NM_000252.2(MTM1): c.1190A > G | 4534 | MTM1 | [′GCCATGCTGATGTTGGATAGCTTCTRTAGG |
| | (p.Tyr397Cys) | | | AGCATTGAAGGGTTCGAAATA′] |
| 3719 | NM_000252.2(MTM1): | 4534 | MTM1 | [′TAATTAAAACAAATTATCTTCATCARTTTA |
| | c.1261 − 10A > G | | | TTCAGCGAATAGGTCATGGTG′] |
| 3720 | NM_001015877.1(PHF6): | 84295 | PHF6 | [′AGGAAAACTGCATATATTTAATGCCRAGA |
| | c.700A > G (p.Lys234Glu) | | | AGGCAGCTGCCCATTATAAGTG′] |
| 3721 | NM_001015877.1(PHF6): | 84295 | PHF6 | [′GAAAATGAAGCACGAGGAAAACTGCRTAT |
| | c.686A > G (p.His229Arg) | | | ATTTAATGCCAAGAAGGCAGCT′] |
| 3722 | NM_001015877.1(PHF6): | 84295 | PHF6 | [′CACAGTCCAGCTCACAACAACATCARGAG |
| | c.769A > G (p.Arg257Gly) | | | CAGAATTTGGAGACTTTGATAT′] |
| 3723 | NM_000117.2(EMD): c.1A > G | 2010 | EMD | [′GCCTCCGCCTGAGCCCGCACCCGCCRTGG |
| | (p.Met1Val) | | | ACAACTACGCAGATCTTTCGGA′] |
| 3724 | NM_004006.2(DMD): c.2317A > G | 1756 | DMD | [′GGCCATAGAGCGAGAAAAAGCTGAGRAGT |
| | (p.Lys773Glu) | | | TCAGAAAACTGCAAGATGCCAG′] |
| 3725 | NM_004006.2(DMD): c.8734A > G | 1756 | DMD | [′TCTACGAAAGCAGGCTGAGGAGGTCRATA |
| | (p.Asn2912Asp) | | | CTGAGTGGGAAAAATTGAACCT′] |
| 3726 | NM_004006.2(DMD): c.8762A > G | 1756 | DMD | [′ACTGAGTGGGAAAAATTGAACCTGCRCTC |
| | (p.His2921Arg) | | | CGCTGACTGGCAGAGAAAAATA′] |
| 3727 | NM_004006.2(DMD): c.835A > G | 1756 | DMD | [′TCCCCCAAACCCTTCTCTGCAGATCRCGGT |
| | (p.Thr279Ala) | | | CAGTCTAGCACAGGGATATGA′] |
| 3728 | NM_004006.2(DMD): | 1756 | DMD | [′TCGATCGCACTTCAGTTATGATAAAYTGAC |
| | c.9225 − 285A > G | | | CTTGTTATGTGATCAATAATC′] |
| 3729 | NM_000033.3(ABCD1): c.443A > G | 215 | ABCD1 | [′ATCGCCCTCCCTGCTACCTTCGTCARCAGT |
| | (p.Asn148Ser) | | | GCCATCCGTTACCTGGAGGGC′] |
| 3730 | NM_003588.3(CUL4B): | 8450 | CUL4B | [′TTTGATTTCTTTTTTTTTCATTGGCRGATCA |
| | c.901 − 2A > G | | | TGATCAGGAGCATTTTTTTG′] |
| 3731 | NM_000061.2(BTK): c.1288A > G | 695 | BTK | [′GAGAGGCCAGTACGACGTGGCCATCRAGA |
| | (p.Lys430Glu) | | | TGATCAAAGAAGGCTCCATGTC′] |
| 3732 | NM_000061.2(BTK): c.1082A > G | 695 | BTK | [′AGCACCATCCCTGAGCTCATTAACTRCCAT |
| | (p.Tyr361Cys) | | | CAGCACAACTCTGCAGGTGAG′] |
| 3733 | NM_000061.2(BTK): c.919A > G | 695 | BTK | [′GGGGAAAGAAGGAGGTTTCATTGTCRGAG |
| | (p.Arg307Gly) | | | ACTCCAGCAAAGCTGGCAAATA′] |
| 3734 | NM_000061.2(BTK): c.1766A > G | 695 | BTK | [′CACCTTCTAGGGGTTTTGATGTGGGRAATT |
| | (p.Glu589Gly) | | | TACTCCCTGGGGAAGATGCCA′] |
| 3735 | NM_003413.3(ZIC3): c.1213A > G | 7547 | ZIC3 | [′CTACACGCACCCGAGCTCCCTGCGCRAAC |
| | (p.Lys405Glu) | | | ACATGAAGGTAATTACCTCTTT′] |
| 3736 | NM_001037811.2(HSD17B10): | 3028 | HSD17B10 | [′GCCATCATCGAGAACCCATTCCTCARTGGA |
| | c.713A > G (p.Asn238Ser) | | | GAGGTCATCCGGCTGGATGGG′] |
| 3737 | NM_003639.4(IKBKG): | 8517 | IKBKG | [′CAAGTGCCAGTATCAGGCCCCTGATRTGG |
| | c.1219A > G (p.Met407Val) | | | ACACCCTGCAGATACATGTCAT′] |
| 3738 | NM_003639.4(IKBKG): | 8517 | IKBKG | [′ATACATGTCATGGAGTGCATTGAGTRGGG |
| | c.1259A > G (p.Ter420Trp) | | | CCGGCCAGTGCAAGGCCACTGC′] |
| 3739 | NM_005448.2(BMP15): c.704A > G | 9210 | BMP15 | [′TTGGACATTGCCTTCTTGTTACTCTRTTTCA |
| | (p.Tyr235Cys) | | | ATGATACTCATAAAAGCATT′] |
| 3740 | NM_005120.2(MED12): | 9968 | MED12 | [′AAGGTGAAGAACACCATCTACTGCARCGT |
| | c.3020A > G (p.Asn1007Ser) | | | GGAGCCATCGGAATCAAATATG′] |
| 3741 | NM_003688.3(CASK): c.2129A > G | 8573 | CASK | [′GGCAAGAAAAAGAAGCAGTACAAAGRTA |
| | (p.Asp710Gly) | | | AATATTTGGCAAAGCACAATGCA′] |
| 3742 | NM_001363.4(DKC1): c.361A > G | 1736 | DKC1 | [′ACTTCGGGTGGAGAAGACAGGGCACRGTG |
| | (p.Ser121Gly) | | | GTACTCTGGATCCCAAGGTGAC′] |
| 3743 | NM_001363.4(DKC1): c.1069A > G | 1736 | DKC1 | [′ATTAATGACCACAGCGGTCATCTCTRCCTG |
| | (p.Thr357Ala) | | | CGACCATGGTATAGTAGCCAA′] |
| 3744 | NM_000307.4(POU3F4): | 5456 | POU3F4 | [′GTTCTGTAATCGAAGACAAAAAGAGRAAA |
| | c.1000A > G (p.Lys334Glu) | | | GAATGACTCCGCCAGGGGATCA′] |
| 3745 | NM_004429.4(EFNB1): c.472A > G | 1947 | EFNB1 | [′GGGCGGTGTGTGCCGCACACGCACCRTGA |
| | (p.Met158Val) | | | AGATCATCATGAAGGTTGGGCA′] |
| 3746 | NM_000489.4(ATRX): c.4826A > G | 546 | ATRX | [′TTCTTATAGGTGGTAAGTTTTCTTCRTACA |
| | (p.His1609Arg) | | | GTTCTTTTGTGTGACAAACTG′] |
| 3747 | NM_000489.4(ATRX): c.5579A > G | 546 | ATRX | [′TTCTTTTGATCAGGTGTGGGCAATAVTAGT |
| | (p.Asn1860Ser) | | | GAAGGTGGAAGAGGAAAGGCA′] |
| 3748 | NM_000489.4(ATRX): c.6488A > G | 546 | ATRX | [′GGACAAACTAAGCCTGTTTATGTATRTAGG |
| | (p.Tyr2163Cys) | | | TTCTTAGCTCAGGTAGGTTTA′] |
| 3749 | NM_000489.4(ATRX): c.6811A > G | 546 | ATRX | [′AGAAGAAGAGTTGACTGAAGAAGAARGA |
| | (p.Arg2271Gly) | | | AAAGCAGCTTGGGCTGAGTATGA′] |
| 3750 | NM_000052.6(ATP7A): c.3911A > G | 538 | ATP7A | [′GTAGCAATGGTGGGAGATGGAATCARTGA |
| | (p.Asn1304Ser) | | | CTCCCCAGCTCTGGCAATGGCT′] |
| 3751 | NM_004992.3(MECP2): c.410A > G | 4204 | MECP2 | [′GGAAAAGCCTTTCGCTCTAAAGTGGRGTT |
| | (p.Glu137Gly) | | | GATTGCGTACTTCGAAAAGGTA′] |
| 3752 | NM_000137.2(FAH): c.1141A > G | 2184 | FAH | [′CATAGACCTGGGGAATGGTCAGACCRGGA |
| | (p.Arg381Gly) | | | AGTTTCTGCTGGACGGGGATGA′] |
| 3753 | NM_000137.2(FAH): c.836A > G | 2184 | FAH | [′CCCTTTGCTGTGCCCAACCCGAAGCRGGTA |
| | (p.Gln279Arg) | | | AGCACATTCTCTGCAGGAAGC′] |
| 3754 | NM_002769.4(PRSS1): c.68A > G | −1 | — | [′GCTGCCCCCTTTGATGATGATGACARGATC |
| | (p.Lys23Arg) | | | GTTGGGGGCTACAACTGTGAG′] |
| 3755 | NM_002769.4(PRSS1): c.161A > G | −1 | — | [′CACTTCTGTGGTGGCTCCCTCATCARCGAA |
| | (p.Asn54Ser) | | | CAGTGGGTGGTATCAGCAGGC′] |
| 3756 | NM_000373.3(UMPS): c.286A > G | 7372 | UMPS | [′AACCAATCAAATTCCAATGCTTATTRGAAG |
| | (p.Arg96Gly) | | | GAAAGAAACAAAGGATTATGG′] |
| 3757 | NM_001918.3(DBT): c.1355A > G | 1629 | DBT | [′ATGAATGTGAGCTGGTCAGCTGATCRCAG |
| | (p.His452Arg) | | | AGTTATTGATGGTGCTACAATG′] |
| 3758 | NM_000108.4(DLD): c.214A > G | 1738 | DLD | [′TTGGTTGTAGACAGTCTGCATTGAGRAAA |
| | (p.Lys72Glu) | | | ATGAAACACTTGGTGGAACATG′] |
| 3759 | NM_000108.4(DLD): c.1483A > G | 1738 | DLD | [′CTTGCAGACCTTATCAGAAGCTTTTRGAGA |
| | (p.Arg495Gly) | | | AGCAAATCTTGCTGCGTCATT′] |
| 3760 | NM_000108.4(DLD): c.1081A > G | 1738 | DLD | [′CATTGGTGATGTAGTTGCTGGTCCARTGCT |
| | (p.Met361 Val) | | | GGCTCACAAAGCAGAGGATGA′] |
| 3761 | NM_000481.3(AMT): c.125A > G | 275 | AMT | [′CGCAGGACACCGCTCTATGACTTCCRCCTG |
| | (p.His42Arg) | | | GCCCACGGCGGGAAAATGGTG′] |
| 3762 | NM_000151.3(G6PC): | 2538 | G6PC | [′GGCTCAACCTCGTCTTTAAGTGGTARGAAC |
| | c.230 + 4A > G | | | CATATAGAGAGGAGATCAGCA′] |
| 3763 | NM_000532.4(PCCB): c.1304A > G | 5096 | PCCB | [′CAGCATTTGGATCTGTTTTAGGCCTRTGGA |
| | (p.Tyr435Cys) | | | GGTGCCTATGATGTCATGAGC′] |
| 3764 | NM_000387.5(SLC25A20): | 788 | SLC25A20 | [′CCAGATGTGCTCAAGTCTCGATTCCRGACT |
| | c.713A > G (p.Gln238Arg) | | | GGTGAGTGGAAGGTAGTGGGG′] |
| 3765 | NM_004360.3(CDH1): c.2512A > G | 999 | CDH1 | [′TCTGCTCGTGTTTGACTATGAAGGARGCGG |
| | (p.Ser838Gly) | | | TTCCGAAGCTGCTAGTCTGAG′] |
| 3766 | NM_004360.3(CDH1): c.1018A > G | 999 | CDH1 | [′GTCGATCTCTCTGCAGAGTTTCCCTDCGTA |
| | (p.Thr340Ala) | | | TACCCTGGTGGTTCAAGCTGC′] |
| 3767 | NM_003361.3(UMOD): c.383A > G | 7369 | UMOD | [′TGCCACGCCCTGGCCACATGTGTCARTGTG |
| | (p.Asn128Ser) | | | GTGGGCAGCTACTTGTGCGTA′] |
| 3768 | NM_000463.2(UGT1A1): | −1 | — | [′GCTGATGCTTTGGGCAAAATCCCTCRGACA |
| | c.992A > G (p.Gln331Arg) | | | GTAAGAAGATTCTATACCATG′] |
| 3769 | NM_000463.2(UGT1A1): | −1 | — | [′TGACATCCTCCCTATTTTGCATCTCRGGTC |
| | c.1085 − 2A > G | | | ACCCGATGACCCGTGCCTTTA′] |
| 3770 | NM_000463.2(UGT1A1): c.1070A > | −1 | — | [′ACGATACTTGTTAAGTGGCTACCCCRAAAC |
| | G (p.Gln357Arg) | | | GATCTGCTTGGTATGTTGGGC′] |
| 3771 | NM_000463.2(UGT1A1): c.1198A > | −1 | — | [′GCCCTTGTTTGGTGATCAGATGGACVATGC |
| | G (p.Asn400Asp) | | | AAAGCGCATGGAGACTAAGGG′] |
| 3772 | NM_001382.3(DPAGT1): | 1798 | DPAGT1 | [′TCTCTCCCCGCAGGAATCCTGTACTRTGTC |
| | c.509A > G (p.Tyr170Cys) | | | TACATGGGGCTGCTGGCAGTG′] |
| 3773 | NM_001007792.1(NTRK1): | 4914 | NTRK1 | [′CCCACCCACGTCAACAACGGCAACTRCAC |
| | c.986A > G (p.Tyr329Cys) | | | GCTGCTGGCTGCCAACCCCTTC′] |
| 3774 | NM_001007792.1(NTRK1): | 4914 | NTRK1 | [′CTGCACCGAGGGCCGCCCCCTGCTCRTGGT |
| | c.1651A > G (p.Met551 Val) | | | CTTTGAGTATATGCGGCACGG′] |
| 3775 | NM_000363.4(TNNI3): c.569A > G | 7137 | TNNI3 | [′CTCCAGGAAAACCGGGAGGTGGGAGRCTG |
| | (p.Asp190Gly) | | | GCGCAAGAACATCGATGCACTG′] |
| 3776 | NM_000363.4(TNNI3): c.532A > G | 7137 | TNNI3 | [′GCGGGCCCACCTCAAGCAGGTGAAGRAGG |
| | (p.Lys178Glu) | | | AGGACACCGAGAAGGTGAGTGT′] |
| 3777 | NM_001018005.1(TPM1): | 7168 | TPM1 | [′ATTGAGAGCGACCTGGAACGTGCAGDGGA |
| | c.539A > G (p.Glu180Gly) | | | GCGGGCTGAGCTCTCAGAAGGG′] |
| 3778 | NM_001159287.1(TPI1): | 7167 | TPI1 | [′CCTGGCCTATGAGCCTGTGTGGGCCRTTGG |
| | c.622A > G (p.Ile208Val) | | | TACTGGCAAGACTGCAACACC′] |
| 3779 | NM_000359.2(TGM1): c.1469A > G | 7051 | TGM1 | [′AATGGCCTGGTCTACATGAAGTACGRCAC |
| | (p.Asp490Gly) | | | GCCTTTCATTTTTGCTGAGGTG′] |
| 3780 | NM_001024847.2(TGFBR2): | 7048 | TGFBR2 | [′CCCTGTGTTTGCTGGCTTTCTTCACRGAAG |
| | c.1472 − 2A > G | | | TAAAAGATTATGAGCCTCCAT′] |
| 3781 | NM_003242.5(TGFBR2): | 7048 | TGFBR2 | [′GCTATAGGTGGGAACTGCAAGATACRTGG |
| | c.1273A > G (p.Met425Val) | | | CTCCAGAAGTCCTAGAATCCAG′] |
| 3782 | NM_004612.3(TGFBR1): | 7046 | TGFBR1 | [′CATTTTGAATCCTTCAAACGTGCTGRCATC |
| | c.1199A > G (p.Asp400Gly) | | | TATGCAATGGGCTTAGTATTC′] |
| 3783 | NM_001128177.1(THRB): | 7068 | THRB | [′CTGCCATGCCAGCCGCTTCCTGCACRTGAA |
| | c.1324A > G (p.Met442Val) | | | GGTGGAATGCCCCACAGAACT′] |
| 3784 | NM_001128177.1(THRB): | 7068 | THRB | [′CCATGCCAGCCGCTTCCTGCACATGRAGGT |
| | c.1327A > G (p.Lys443Glu) | | | GGAATGCCCCACAGAACTCTT′] |
| 3785 | NM_001128177.1(THRB): | 7068 | THRB | [′AACCTTGAATGGGGAAATGGCAGTGRCAC |
| | c.1009A > G (p.Thr337Ala) | | | GGGGCCAGCTGAAAAATGGGGG′] |
| 3786 | NM_033360.3(KRAS): c.13A > G | 3845 | KRAS | [′AGGCCTGCTGAAAATGACTGAATATRAAC |
| | (p.Lys5Glu) | | | TTGTGGTAGTTGGAGCTGGTGG′] |
| 3787 | NM_005343.2(HRAS): c.350A > G | −1 | — | [′GTGCCCATGGTGCTGGTGGGGAACARGTG |
| | (p.Lys117Arg) | | | TGACCTGGCTGCACGCACTGTG′] |
| 3788 | NM_001063.3(TF): c.956A > G | 7018 | TF | [′GACCTGCTGTTTAAGGACTCTGCCCRCGGG |
| | (p.His319Arg) | | | TTTTTAAAAGTCCCCCCCAGG′] |
| 3789 | NM_001063.3(TF): c.1936A > G | 7018 | TF | [′CTTTTGTTTGTTCCGGTCGGAAACCRAGGA |
| | (p.Lys646Glu) | | | CCTTCTGTTCAGAGATGACAC′] |
| 3790 | NM_212472.2(PRKAR1A): | 5573 | PRKAR1A | [′ATGAGTTCTTCATTATTTTAGAGGTRAAGA |
| | c.891+ 3A > G | | | ACTCAGAATTTAATACTTGAA′] |
| 3791 | NM_212472.2(PRKAR1A): c.1A > G | 5573 | PRKAR1A | [′GTGTGTTTTTTTCTCGCAGAGAACCRTGGA |
| | (p.Met1Val) | | | GTCTGGCAGTACCGCCGCCAG′] |
| 3792 | NM_000362.4(TIMP3): c.572A > G | −1 | — | [′GCCTGCATCCGGCAGAAGGGCGGCTRCTG |
| | (p.Tyr191Cys) | | | CAGCTGGTACCGAGGATGGGCC′] |
| 3793 | NM_000367.3(TPMT): c.719A > G | 7172 | TPMT | [′GATTACAGGTGTGAGCCACCGCACCNAGC |
| - | (p.Tyr240Cys) | | | CAATTTTGAGTATTTTTAAAAG′, |
| 3795 | | | | ′TAACATGTTACTCTTTCTTGTTTCARGTAAA |
| | | | | ATATGCAATATACRTYGTCT′, |
| | | | | ′AGGTTGATGCTTTTGAAGAACGACAKAAAA |
| | | | | GTTGGGGAATTGACTGTCTTT′] |
| 3796 | NM_198253.2(TERT): c.2315A > G | 7015 | TERT | [′TCTACCTTGACAGACCTCCAGCCGTRCATG |
| | (p.Tyr772Cys) | | | CGACAGTTCGTGGCTCACCTG′] |
| 3797 | NM_000073.2(CD3G): c.1A > G | 917 | CD3G | [′CTTTTGCCGGAGGACAGAGACTGACRTGG |
| | (p.Met1Val) | | | AACAGGGGAAGGGCCTGGCTGT′] |
| 3798 | NM_000899.4(KITLG): c.107A > G | 4254 | KITLG | [′ATCTGCAGGAATCGTGTGACTAATARTGTA |
| | (p.Asn36Ser) | | | AAAGACGTCACTAAATTGGTA′] |
| 3799 | NM_001024858.2(SPTB): c.1A > G | 6710 | SPTB | [′GCGGAGCTGCTAAGAGCCTGCTGACRTGA |
| | (p.Met1Val) | | | CATCGGCCACAGAGTTTGAAAA′] |
| 3800 | NM_003126.2(SPTA1): c.143A > G | 6708 | SPTA1 | [′GAGCGGGTCGCTGAGAGGGGTCAGARGCT |
| | (p.Lys48Arg) | | | TGAGGATTCCTATCACTTACAA′] |
| 3801 | NM_005633.3(SOS1): c.1654A > G | 6654 | SOS1 | [′TTTACAGTACCGGAGTACACTGGAARGGA |
| | (p.Arg552Gly) | | | TGCTTGATGTAACAATGCTACA′] |
| 3802 | NM_003041.3(SLC5A2): | −1 | — | [′GACCCGAGCTGGGCCCGTGTGGTCARCCT |
| | c.1961A > G (p.Asn654Ser) | | | CAATGCCCTGCTCATGATGGCA′] |
| 3803 | NM_000343.3(SLC5A1): c.83A > G | 6523 | SLC5A1 | [′CACGAGCTCATTCGCAATGCAGCCGRTATC |
| | (p.Asp28Gly) | | | TCCATCATCGTTATCTACTTC′] |
| 3804 | NM_000702.3(ATP1A2): | 477 | ATP1A2 | [′TCTCTACCAGGTGTGCCTGACCCTGRCAGC |
| | c.1033A > G (p.Thr345Ala) | | | CAAGCGCATGGCACGGAAGAA′] |
| 3805 | NM_003124.4(SPR): c.448A > G | 6697 | SPR | [′CTTCCCGGACAGTCCTGGCCTCAACRGAAC |
| | (p.Arg150Gly) | | | CGTGGTTAACATCTCGTCCCT′] |
| 3806 | NM_001161766.1(AHCY): | 191 | AHCY | [′CTCACCAACCTCATCCACACCAAGTRCCCG |
| | c.344A > G (p.Tyr115Cys) | | | CAGCTTCTGCCAGGTGAGCAG′] |
| 3807 | NM_001035.2(RYR2): | 6262 | RYR2 | [′TGCGAGGACACCATCTTTGAAATGCRGCT |
| | c.12602A > G (p.Gln4201Arg) | | | GGCGGCTCAGATCTCGGAGTCG′] |
| 3808 | NM_000540.2(RYR1): | 6261 | RYR1 | [′CAGTCCTTCCTGTACCTGGGCTGGTRTATG |
| | c.14387A > G (p.Tyr4796Cys) | | | GTGATGTCCCTCTTGGGACAC′] |
| 3809 | NM_000540.2(RYR1): | 6261 | RYR1 | [′CTCTGTCTCAAAAAAAAAAAAAAACRTAT |
| | c.14647 − 1449A > G | | | GTAAAGTTGTTCCCAAATGCCA′] |
| 3810 | NM_000540.2(RYR1): | 6261 | RYR1 | [′GGTGTACTACTTCCTGGAGGAAAGCRCAG |
| | c.13909A > G (p.Thr4637Ala) | | | GCTACATGGAACCCGCCCTGCG′] |
| 3811 | NM_000539.3(RHO): c.533A > G | 6010 | RHO | [′TCTCCCTACCTGCCTGTCCTCAGGTRCATC |
| | (p.Tyr178Cys) | | | CCCGAGGGCCTGCAGTGCTCG′] |
| 3812 | NM_000539.3(RHO): c.569A > G | 6010 | RHO | [′GGCCTGCAGTGCTCGTGTGGAATCGRCTAC |
| | (p.Asp190Gly) | | | TACACGCTCAAGCCGGAGGTC′] |
| 3813 | NM_000539.3(RHO): c.886A > G | 6010 | RHO | [′CATGACCATCCCAGCGTTCTTTGCCRAGAG |
| | (p.Lys296Glu) | | | CGCCGCCATCTACAACCCTGT′] |
| 3814 | NM_000539.3(RHO): c.44A > G | 6010 | RHO | [′CCTAACTTCTACGTGCCCTTCTCCARTGCG |
| | (p.Asn15Ser) | | | ACGGGTGTGGTACGCAGCCCC′] |
| 3815 | NM_000321.2(RB1): | 5925 | RB1 | [′AAGGGTGCTGTGGATCAGGGAAATGRTGA |
| | c.2490 − 1398A > G | | | GTATGAAGCTGTTTTAAATTCT′] |
| 3816 | NM_000536.3(RAG2): c.115A > G | 5897 | RAG2 | [′CTTTGGACAAAAAGGCTGGCCCAAARGAT |
| | (p.Arg39Gly) | | | CCTGCCCCACTGGAGTTTTCCA′] |
| 3817 | NM_000448.2(RAG1): c.2735A > G | 5896 | RAG1 | [′GAGTGCCCAGAATCCCTCTGCCAGTRCAGT |
| | (p.Tyr912Cys) | | | TTCAATTCACAGCGTTTTGCT′] |
| 3818 | NM_000448.2(RAG1): c.1286A > G | 5896 | RAG1 | [′TTTGCTGACAAAGAAGAAGGTGGAGRTGT |
| | (p.Asp429Gly) | | | GAAGTCCGTGTGCATGACCTTG′] |
| 3819 | NM_000925.3(PDHB): c.395A > G | 5162 | PDHB | [′ATAAACTCAGCTGCCAAGACCTACTRCAT |
| | (p.Tyr132Cys) | | | GTCTGGTGGCCTTCAGCCTGTG′] |
| 3820 | NM_000055.2(BCHE): c.293A > G | 590 | BCHE | [′GCAAATTCTTGCTGTCAGAACATAGRTCAA |
| | (p.Asp98Gly) | | | AGTTTTCCAGGCTTCCATGGA′] |
| 3821 | NM_000055.2(BCHE): c.467A > G | 590 | BCHE | [′ACTGGAACATCATCTTTACATGTTTDTGAT |
| | (p.Tyr156Cys) | | | GGCAAGTTTCTGGCTCGGGTT′] |
| 3822 | NM_005360.4(MAF): c.890A > G | 4094 | MAF | [′CTGAAGCAGAAGAGGCGGACCCTGARAAA |
| | (p.Lys297Arg) | | | CCGCGGCTATGCCCAGTCCTGC′] |
| 3823 | NM_002739.3(PRKCG): c.380A > G | 5582 | PRKCG | [′TCCCTCCTCTACGGGCTTGTGCACCRGGGC |
| | (p.Gln127Arg) | | | ATGAAATGCTCCTGTGAGTGA′] |
| 3824 | NM_002739.3(PRKCG): | 5582 | PRKCG | [′CTTCCTCATGGTTCTAGGAAAAGGCRGTTT |
| | c.1081A > G (p.Ser361Gly) | | | TGGGAAGGTTGGATTCCTGGG′] |
| 3825 | NM_000141.4(FGFR2): c.983A > G | 2263 | FGFR2 | [′ACGGACAAAGAGATTGAGGTTCTCTRTATT |
| | (p.Tyr328Cys) | | | CGGAATGTAACTTTTGAGGAC′] |
| 3826 | NM_000141.4(FGFR2): | 2263 | FGFR2 | [′AAGGAGATTACAGCTTCCCCAGACTRCCT |
| | c.1124A > G (p.Tyr375Cys) | | | GGAGATAGCCATTTACTGCATA′] |
| 3827 | NM_000141.4(FGFR2): c.874A > G | 2263 | FGFR2 | [′TGCCCAGCCCCACATCCAGTGGATCRAGC |
| | (p.Lys292Glu) | | | ACGTGGAAAAGAACGGCAGTAA′] |
| 3828 | NM_000141.4(FGFR2): | 2263 | FGFR2 | [′ACTGTTCATAGATGATGCCACAGAGRAAG |
| | c.1576A > G (p.Lys526Glu) | | | ACCTTTCTGATCTGGTGTCAGA′] |
| 3829 | NM_000313.3(PROS1): c.773A > G | 5627 | PROS1 | [′AACATGTGTGCTCAGCTTTGTGTCARTTAC |
| | (p.Asn258Ser) | | | CCTGGAGGTTACACTTGCTAT′] |
| 3830 | NM_000313.3(PROS1): c.586A > G | 5627 | PROS1 | [′AAATGGTTTTGTTATGCTTTCAAATRAGAA |
| | (p.Lys196Glu) | | | AGATTGTAAAGGTAAGAGCAG′] |
| 3831 | NM_002834.3(PTPN11): | 5781 | PTPN11 | [′GCCTGTTTCAGATTACATCAATGCARATAT |
| | c.922A > G (p.Asn308Asp) | | | CATCATGGTAAGCTTTGCTTT′] |
| 3832 | NM_002834.3(PTPN11): | 5781 | PTPN11 | [′CCTGTTTCAGATTACATCAATGCAAVTATC |
| | c.923A > G (p.Asn308Ser) | | | ATCATGGTAAGCTTTGCTTTT′] |
| 3833 | NM_002834.3(PTPN11): | 5781 | PTPN11 | [′CAAGAAAACAAAAACAAAAATAGATVTA |
| | c.836A > G (p.Tyr279Cys) | | | AAAACATCCTGCCCTGTAAGTAT′] |
| 3834 | NM_002834.3(PTPN11): | 5781 | PTPN11 | [′CACATCAAGATTCAGAACACTGGTGNTTA |
| | c.182A > G (p.Asp61Gly) | | | CTATGACCTGTATGGAGGGGAG′] |
| 3835 | NM_002834.3(PTPN11): | 5781 | PTPN11 | [′AAGATTCAGAACACTGGTGATTACTRTGA |
| | c.188A > G (p.Tyr63Cys) | | | CCTGTATGGAGGGGAGAAATTT′] |
| 3836 | NM_002834.3(PTPN11): | 5781 | PTPN11 | [′GGGGAGAAATTTGCCACTTTGGCTGNGTT |
| | c.227A > G (p.Glu76Gly) | | | GGTCCAGTATTACATGGAACAT′] |
| 3837 | NM_002834.3(PTPN11): | 5781 | PTPN11 | [′TTTGCCACTTTGGCTGAGTTGGTCCRGTAT |
| | c.236A > G (p.Gln79Arg) | | | TACATGGAACATCACGGGCAA′] |
| 3838 | NM_002834.3(PTPN11): | 5781 | PTPN11 | [′TCAGGGATGGTCCAGACAGAAGCACVGTA |
| | c.1529A > G (p.Gln510Arg) | | | CCGATTTATCTATATGGCGGTC′] |
| 3839 | NM_002755.3(MAP2K1): | 5604 | MAP2K1 | [′AACTCTCCGTACATCGTGGGCTTCTRTGGT |
| | c.389A > G (p.Tyr130Cys) | | | GCGTTCTACAGCGATGGCGAG′] |
| 3840 | NM_006006.4(ZBTB16): | 7704 | ZBTB16 | [′CCAGCGCTCCCGGGACTACTCGGCCRTGAT |
| | c.1849A > G (p.Met617Val) | | | CAAGCACCTGAGAACGCACAA′] |
| 3841 | NM_000311.3(PRNP): c.385A > G | 5621 | PRNP | [′AGTGGTGGGGGGCCTTGGCGGCTACRTGC |
| | (p.Met129Val) | | | TGGGAAGTGCCATGAGCAGGCC′] |
| 3842 | NM_000311.3(PRNP): c.650A > G | 5621 | PRNP | [′GTGGTTGAGCAGATGTGTATCACCCRGTAC |
| | (p.Gln217Arg) | | | GAGAGGGAATCTCAGGCCTAT′] |
| 3843 | NM_000311.3(PRNP): c.547A > G | 5621 | PRNP | [′CTTTGTGCACGACTGCGTCAATATCRCAAT |
| | (p.Thr183Ala) | | | CAAGCAGCACACGGTCACCAC′] |
| 3844 | NM_000311.3(PRNP): c.560A > G | 5621 | PRNP | [′TGCGTCAATATCACAATCAAGCAGCRCAC |
| | (p.His187Arg) | | | GGTCACCACAACCACCAAGGGG′] |
| 3845 | NM_000371.3(TTR): c.401A > G | 7276 | TTR | [′ACCATTGCCGCCCTGCTGAGCCCCTRCTCC |
| | (p.Tyr134Cys) | | | TATTCCACCACGGCTGTCGTC′] |
| 3846 | NM_000371.3(TTR): c.238A > G | 7276 | TTR | [′GTCTGGAGAGCTGCATGGGCTCACARCTG |
| | (p.Thr80Ala) | | | AGGAGGAATTTGTAGAAGGGAT′] |
| 3847 | NM_000371.3(TTR): c.185A > G | 7276 | TTR | [′AGAAAGGCTGCTGATGACACCTGGGRGCC |
| | (p.Glu62Gly) | | | ATTTGCCTCTGGGTAAGTTGCC′] |
| 3848 | NM_000371.3(TTR): c.205A > G | 7276 | TTR | [′CCAGACTTTCACACCTTATAGGAAARCCA |
| | (p.Thr69Ala) | | | GTGAGTCTGGAGAGCTGCATGG′] |
| 3849 | NM_000371.3(TTR): c.379A > G | 7276 | TTR | [′CGACTCCGGCCCCCGCCGCTACACCRTTGC |
| | (p.Ile127Val) | | | CGCCCTGCTGAGCCCCTACTC′] |
| 3850 | NM_000371.3(TTR): c.113A > G | 7276 | TTR | [′TGTCCTCTGATGGTCAAAGTTCTAGRTGCT |
| | (p.Asp38Gly) | | | GTCCGAGGCAGTCCTGCCATC′] |
| 3851 | NM_000217.2(KCNA1): c.676A > G | 3736 | KCNA1 | [′CACAGACCCCTTCTTCATCGTGGAARCGCT |
| | (p.Thr226Ala) | | | GTGTATCATCTGGTTCTCCTT′] |
| 3852 | NM_000217.2(KCNA1): c.763A > G | 3736 | KCNA1 | [′GACGGACTTCTTCAAAAACATCATGRACTT |
| | (p.Asn255Asp) | | | CATAGACATTGTGGCCATCAT′] |
| 3853 | NM_002693.2(POLG): c.2864A > G | 5428 | POLG | [′AAAATCTTCAACTACGGCCGCATCTRTGGT |
| | (p.Tyr955Cys) | | | GCTGGGCAGCCCTTTGCTGAG′] |
| 3854 | NM_002693.2(POLG): c.2591A > G | 5428 | POLG | [′GAGCCCACATGGCTCACCGCCAGCARTGC |
| | (p.Asn864Ser) | | | CCGGGTATGTGACCTCTGTACC′] |
| 3855 | NM_000174.4(GP9): c.182A > G | 2815 | GP9 | [′ACCCGCCACCTTCTGCTGGCCAACARCAGC |
| | (p.Asn61Ser) | | | CTTCAGTCCGTGCCCCCGGGA′] |
| 3856 | NM_000174.4(GP9): c.110A > G | 2815 | GP9 | [′CTGGAAACCATGGGGCTGTGGGTGGRCTG |
| | (p.Asp37Gly) | | | CAGGGGCCACGGACTCACGGCC′] |
| 3857 | NM_006206.4(PDGFRA): | 5156 | PDGFRA | [′CCTGGTCATTTATAGAAACCGAGGTRTGA |
| | c.1664A > G (p.Tyr555Cys) | | | AATTCGCTGGAGGGTCATTGAA′] |
| 3858 | NM_000301.3(PLG): c.112A > G | 5340 | PLG | [′GGGGGCTTCACTGTTCAGTGTCACTRAGAA |
| | (p.Lys38Glu) | | | GCAGCTGGGAGCAGGAAGTAT′] |
| 3859 | NM_000293.2(PHKB): | 5257 | PHKB | [′GTTTCATGAGTTATCTCTCTCACCCRGGCG |
| | c.306 − 2A > G | | | AATTGATGATGACAAGGGAAG′] |
| 3860 | NM_000175.3(GPI): c.1028A > G | 2821 | GPI | [′ACACACGCCATGCTGCCCTATGACCRGTAC |
| | (p.Gln343Arg) | | | CTGCACCGCTTTGCTGCGTAC′] |
| 3861 | NM_002633.2(PGM1): c.343A > G | 5236 | PGM1 | [′CAAAGCCATTGGTGGGATCATTCTGRCAG |
| | (p.Thr115Ala) | | | CCAGTCACAACCCAGGGGGCCC′] |
| 3862 | NM_006218.2(PIK3CA): | 5290 | PIK3CA | [′TTCATGAAACAAATGAATGATGCACDTCA |
| | c.3140A > G (p.His1047Arg) | | | TGGTGGCTGGACAACAAAAATG′] |
| 3863 | NM_006218.2(PIK3CA): | 5290 | PIK3CA | [′CGAGATCCTCTCTCTGAAATCACTGVGCAG |
| | c.1634A > G (p.Glu545Gly) | | | GAGAAAGATTTTCTATGGAGT′] |
| 3864 | NM_000478.4(ALPL): c.1250A > G | 249 | ALPL | [′CCCTTCACTGCCATCCTGTATGGCARTGGG |
| | (p.Asn417Ser) | | | CCTGGCTACAAGGTGGTGGGC′] |
| 3865 | NM_018849.2(ABCB4): c.523A > G | 5244 | ABCB4 | [′AGAAATAAACAGGTATAAGATGTGARTTC |
| - | (p.Thr175Ala) | | | AGTCCTCAAATAAACCTACTAT′, |
| 3867 | | | | TTCTATATGAAAGTGTGACATTAACRATGT |
| | | | | ACCTACTCTGTTAGCCGCGYA′, |
| | | | | ′TTTCCTGTCGTAGAATAGCATGAAARAACT |
| | | | | TCTGCCTAATTTTCCTGATCT′] |
| 3868 | NM_001083116.1(PRF1): | 5551 | PRF1 | [′CTGGCCCTGGAAGGGCTCACGGACARCGA |
| | c.755A > G (p.Asn252Ser) | | | GGTGGAGGACTGCCTGACTGTC′] |
| 3869 | NM_198965.1(PTHLH): c.534A > G | 5744 | PTHLH | [′TTCCTTCTTTTTGCAGGAGGCATTGRAATT |
| | (p.Ter178Trp) | | | TTCAGCAGAGACCTTCCAAGG′] |
| 3870 | NM_000316.2(PTH1R): c.668A > G | 5745 | PTH1R | [′CTGCACTGCACGCGCAACTACATCCRCATG |
| | (p.His223Arg) | | | CACCTGTTCCTGTCCTTCATG′] |
| 3871 | NM_003122.4(SPINK1): | 6690 | SPINK1 | [′TTCCATTTTTAGGCCAAATGTTACARTGAA |
| | c.101A > G (p.Asn34Ser) | | | CTTAATGGATGCACCAAGATA′] |
| 3872 | NM_006194.3(PAX9): c.271A > G | 5083 | PAX9 | [′CGTGGTGAAACACATCCGGACCTACRAGC |
| | (p.Lys91Glu) | | | AGAGAGACCCCGGCATCTTCGC′] |
| 3873 | NM_013953.3(PAX8): c.160A > G | −1 | — | [′CGACATCTCTCGCCAGCTCCGCGTCRGCCA |
| | (p.Ser54Gly) | | | TGGCTGCGTCAGCAAGATCCT′] |
| 3874 | NM_005188.3(CBL): c.1144A > G | 867 | CBL | [′GATGGGCTCCACATTCCAACTATGTRAAAT |
| | (p.Lys382Glu) | | | ATGTGCTGAAAATGATAAGGA′] |
| 3875 | NM_000222.2(KIT): c.2459A > G | 3815 | KIT | [′GGTCTAGCCAGAGACATCAAGAATGRTTC |
| | (p.Asp820Gly) | | | TAATTATGTGGTTAAAGGAAAC′] |
| 3876 | NM_000222.2(KIT): c.2386A > G | 3815 | KIT | [′GTGTATTCACAGAGACTTGGCAGCCRGAA |
| | (p.Arg796Gly) | | | ATATCCTCCTTACTCATGGTCG′] |
| 3877 | NM_000222.2(KIT): c.1924A > G | 3815 | KIT | [′ACGGGAAGCCCTCATGTCTGAACTCRAAG |
| | (p.Lys642Glu) | | | TCCTGAGTTACCTTGGTAATCA′] |
| 3878 | NM_001005862.2(ERBB2): | 2064 | ERBB2 | [′CGGAACGTGCTGGTCAAGAGTCCCARCCA |
| | c.2480A > G (p.Asn827Ser) | | | TGTCAAAATTACAGACTTCGGG′] |
| 3879 | NM_001127500.1(MET): | 4233 | MET | [′GATTTTGGTCTTGCCAGAGACATGTRTGAT |
| | c.3743A > G (p.Tyr1248Cys) | | | AAAGAATACTATAGTGTACAC′] |
| 3880 | NM_001127500.1(MET): | 4233 | MET | [′ACAGGGCATTTTGGTTGTGTATATCVTGGG |
| | c.3335A > G (p.His1112Arg) | | | ACTTTGTTGGACAATGATGGC′] |
| 3881 | NM_001127500.1(MET): | 4233 | MET | [′AGTGTACACAACAAAACAGGTGCAARGCT |
| | c.3785A > G (p.Lys1262Arg) | | | GCCAGTGAAGTGGATGGCTTTG′] |
| 3882 | NM_002524.4(NRAS): c.182A > G | 4893 | NRAS | [′TTGGACATACTGGATACAGCTGGACNAGA |
| | (p.Gln61Arg) | | | AGAGTACAGTGCCATGAGAGAC′] |
| 3883 | NM_004333.4(BRAF): c.1801A > G | 673 | BRAF | [′AGGTGATTTTGGTCTAGCTACAGTGVAATC |
| | (p.Lys601Glu) | | | TCGATGGAGTGGGTCCCATCA′] |
| 3884 | NM_004333.4(BRAF): c.1781A > G | 673 | BRAF | [′GAAGACCTCACAGTAAAAATAGGTGDTTT |
| | (p.Asp594Gly) | | | TGGTCTAGCTACAGTGAAATCT′] |
| 3885 | NM_004333.4(BRAF): c.770A > G | 673 | BRAF | [′GACTTTTGTCGAAAGCTGCTTTTCCRGGGT |
| | (p.Gln257Arg) | | | TTCCGCTGTCAAACATGTGGT′] |
| 3886 | NM_004333.4(BRAF): c.1495A > G | 673 | BRAF | [′TACACCTCAGCAGTTACAAGCCTTCRAAA |
| | (p.Lys499Glu) | | | ATGAAGTAGGAGTACTCAGGTG′] |
| 3887 | NM_004333.4(BRAF): c.1502A > G | 673 | BRAF | [′CAGCAGTTACAAGCCTTCAAAAATGNAGT |
| | (p.Glu501Gly) | | | AGGAGTACTCAGGTGAGCTTGT′] |
| 3888 | NM_004333.4(BRAF): c.1741A > G | 673 | BRAF | [′CATCCACAGAGACCTCAAGAGTAATVGTA |
| | (p.Asn581Asp) | | | TCCTTCCTGAAATTTGTCTGCG′] |
| 3889 | NM_000270.3(PNP): c.383A > G | 4860 | PNP | [′CTGAACCCCAAGTTTGAGGTTGGAGRTATC |
| | (p.Asp128Gly) | | | ATGCTGATCCGTGACCATATC′] |
| 3890 | NM_000270.3(PNP): c.575A > G | 4860 | PNP | [′CAACGTGAGCTACAGGAAGGCACCTRTGT |
| | (p.Tyr192Cys) | | | GATGGTGGCAGGCCCCAGCTTT′] |
| 3891 | NM_000258.2(MYL3): c.445A > G | 4634 | MYL3 | [′CGACAAGGAGGGCAATGGCACTGTCRTGG |
| | (p.Met149Val) | | | GTGCTGAGCTTCGCCACGTGCT′] |
| 3892 | NM_000257.3(MYH7): c.2333A > G | 4625 | MYH7 | [′CTGGGGCTGCTGGAGGAAATGAGGGRCGA |
| | (p.Asp778Gly) | | | GAGGCTGAGCCGCATCATCACG′] |
| 3893 | NM_000257.3(MYH7): c.2717A > G | 4625 | MYH7 | [′CTGGCAGATGCTGAGGAGCGCTGTGRTCA |
| | (p.Asp906Gly) | | | GCTGATCAAAAACAAGATTCAG′] |
| 3894 | NM_002470.3(MYH3): c.1385A > G | 4621 | MYH3 | [′AGACAACACTTCATTGGTGTTTTGGRCATT |
| | (p.Asp462Gly) | | | GCAGGCTTTGAAATCTTTGAG′] |
| 3895 | NM_000530.6(MPZ): c.286A > G | 4359 | MPZ | [′CTACATTGACGAGGTGGGGACCTTCRAAG |
| | (p.Lys96Glu) | | | AGCGCATCCAGTGGGTAGGGGA′] |
| 3896 | NM_000530.6(MPZ): c.242A > G | 4359 | MPZ | [′TCCCCTCATTCCTCATAGATCTTCCRCTAT |
| | (p.His81Arg) | | | GCCAAGGGACAACCCTACATT′] |
| 3897 | NG_012123.1: g.2493A > G | 6347 | CCL2 | [′GAAAAGAAAGTCTTCTGGAAAGTGAYAGC |
| | | | | TGTCTGCCTCCCACTTCTGCTC′] |
| 3898 | RMRP: n.71A > G | 6023 | RMRP | [′TGTTCCTCCCCTTTCCGCCTAGGGGRAAGT |
| | | | | CCCCGGACCTCGGGCAGAGAG′] |
| 3899 | NM_005006.6(NDUFS1): | 4719 | NDUFS1 | [′TTTCTCAGAAAGACAGAATCCATTGRTGTA |
| | c.755A > G (p.Asp252Gly) | | | ATGGATGCGGTTGGAAGTAAT′] |
| 3900 | NM_005912.2(MC4R): c.508A > G | 4160 | MC4R | [′GACAGTTAAGCGGGTTGGGATCATCRTAA |
| | (p.Ile170Val) | | | GTTGTATCTGGGCAGCTTGCAC′] |
| 3901 | NM_005912.2(MC4R): c.821A > G | 4160 | MC4R | [′ATATTCTACATCTCTTGTCCTCAGARTCCA |
| | (p.Asn274Ser) | | | TATTGTGTGTGCTTCATGTCT′] |
| 3902 | NM_005912.2(MC4R): c.289A > G | 4160 | MC4R | [′GGCTGATATGCTGGTGAGCGTTTCARATGG |
| | (p.Asn97Asp) | | | ATCAGAAACCATTGTCATCAC′] |
| 3903 | NM_005912.2(MC4R): c.185A > G | 4160 | MC4R | [′CTGGGTGTCATCAGCTTGTTGGAGARTATC |
| | (p.Asn62Ser) | | | TTAGTGATTGTGGCAATAGCC′] |
| 3904 | NM_000900.3(MGP): | 4256 | MGP | [′AAGATTCCATGCTTTCATGTGATTCHGAAA |
| | c.62 − 2A > G | | | TTAAAAAAAAAAGATTCATTA′] |
| 3905 | NM_012064.3(MIP): c.401A > G | 4284 | MIP | [′AGCGTGGGCCAGGCAACCACAGTGGRGAT |
| | (p.Glu134Gly) | | | CTTCCTGACGCTCCAGTTCGTG′] |
| 3906 | NM_000233.3(LHCGR): | −1 | — | [′AAAATGGCAATCCTCATCTTCACCGRTTTC |
| | c.1733A > G (p.Asp578Gly) | | | ACCTGCATGGCACCTATCTCT′] |
| 3907 | NM_000233.3(LHCGR) | −1 | — | [′CCAGAATTAATGGCTACCAATAAAGRTAC |
| | c.1691A > G (p.Asp564Gly) | | | AAAGATTGCTAAGAAAATGGCA′] |
| 3908 | NM_000894.2(LHB): c.221A > G | 3972 | LHB | [′CAGGCGGTCCTGCCGCCCCTGCCTCRGGTG |
| | (p.Gln74Arg) | | | GTGTGCACCTACCGTGATGTG′] |
| 3909 | NM_000238.3(KCNH2): | 3757 | KCNH2 | [′CATGTTCATTGTGGACATCCTCATCRACTT |
| | c.1408A > G (p.Asn470Asp) | | | CCGCACCACCTACGTCAATGC′] |
| 3910 | NM_001754.4(RUNX1): c.328A > G | 861 | RUNX1 | [′GCTGCCTACGCACTGGCGCTGCAACRAGA |
| | (p.Lys110Glu) | | | CCCTGCCCATCGCTTTCAAGGT′] |
| 3911 | NM_170707.3(LMNA): c.608A > G | 4000 | LMNA | [′AACAGGCTGCAGACCATGAAGGAGGDACT |
| | (p.Glu203Gly) | | | GGACTTCCAGAAGAACATCTAC′] |
| 3912 | NM_000421.3(KRT10): | −1 | — | [′TGAATACCAACAACTCCTGGATATTDAGA |
| | c.1315A > G (p.Lys439Glu) | | | TCCGACTGGAGAATGAAATTCA′] |
| 3913 | NM_000421.3(KRT10): | −1 | — | [′CCGCCGCGTCCGCCGCCTCCGGAACYAAA |
| | c. 1374 − 2A > G | | | CGGGGTGAGGTCACATTCGGTT′] |
| 3914 | NM_000422.2(KRT17): c.274A > G | 3872 | KRT17 | [′TGAGAAGGCCACCATGCAGAACCTCVATG |
| | (p.Asn92Asp) | | | ACCGCCTGGCCTCCTACCTGGA′] |
| 3915 | NM_000422.2(KRT17): c.275A > G | 3872 | KRT17 | [′GAGAAGGCCACCATGCAGAACCTCARTGA |
| | (p.Asn92Ser) | | | CCGCCTGGCCTCCTACCTGGAC′] |
| 3916 | NM_005557.3(KRT16): c.374A > G | 3868 | KRT16 | [′GAGAAGGTGACCATGCAGAACCTCARTGA |
| | (p.Asn125Ser) | | | CCGCCTGGCCTCCTACCTGGAC′] |
| 3917 | NM_000526.4(KRT14): c.368A > G | 3861 | KRT14 | [′GAGAAGGTGACCATGCAGAACCTCARTGA |
| | (p.Asn123Ser) | | | CCGCCTGGCCTCCTACCTGGAC′] |
| 3918 | NM_000424.3(KRT5): c.1424A > G | 3852 | KRT5 | [′ATCGCCACTTACCGCAAGCTGCTGGRGGG |
| | (p.Glu475Gly) | | | CGAGGAATGCAGGTGAGTAGAC′] |
| 3919 | NM_000418.3(IL4R): c.223A > G | 3566 | IL4R | [′CTGTGTCTGCAGAGCCCACACGTGTNTCCC |
| | (p.Ile75Val) | | | TGAGAACAACGGAGGCGCGGG′] |
| 3920 | NM_001079817.1(INSR): | 3643 | INSR | [′CCAGGAGAGAAACGACATTGCCCTGRAGA |
| | c.1459A > G (p.Lys487Glu) | | | CCAATGGGGACCAGGCATCCTG′] |
| 3921 | NM_000208.2(INSR): c.707A > G | 3643 | INSR | [′TGCACCGCCGAAGGCCTCTGTTGCCRCAGC |
| | (p.His236Arg) | | | GAGTGCCTGGGCAACTGTTCT′] |
| 3922 | NM_000208.2(INSR): c.1466A > G | 3643 | INSR | [′AGAAACGACATTGCCCTGAAGACCARTGG |
| | (p.Asn489Ser) | | | GGACCAGGCATCCTGTAAGTCA′] |
| 3923 | NM_000208.2(INSR): | 3643 | INSR | [′CTTCTAGCTCAGCTGCCAGATTGTCYAAGG |
| | c.1124 − 2A > G | | | AAAGGAGAGAATATCCAGTGG′] |
| 3924 | NM_000208.2(INSR): c.1372A > G | 3643 | INSR | [′TCAGGGGAAACTCTTCTTCCACTATRACCC |
| | (p.Asn458Asp) | | | CAAACTCTGCTTGTCAGAAAT′] |
| 3925 | NM_000454.4(SOD1): c.131A > G | 6647 | SOD1 | [′ATTAAAGGACTGACTGAAGGCCTGCRTGG |
| | (p.His44Arg) | | | ATTCCATGTTCATGAGTTTGGA′] |
| 3926 | NM_000454.4(SOD1): c.302A > G | 6647 | SOD1 | [′GATGGTGTGGCCGATGTGTCTATTGRAGAT |
| | (p.Glu101Gly) | | | TCTGTGATCTCACTCTCAGGA′] |
| 3927 | NM_000454.4(SOD1): c.140A > G | 6647 | SOD1 | [′CTGACTGAAGGCCTGCATGGATTCCRTGTT |
| | (p.His47Arg) | | | CATGAGTTTGGAGATAATACA′] |
| 3928 | NM_000454.4(SOD1): c.242A > G | 6647 | SOD1 | [′ATCTGATGCTTTTTCATTATTAGGCRTGTT |
| | (p.His81Arg) | | | GGAGACTTGGGCAATGTGACT′] |
| 3929 | NM_000183.2(HADHB): c.788A > G | 3032 | HADHB | [′GCACAGGATGAAGGACTCCTTTCTGRTGTG |
| | (p.Asp263Gly) | | | GTACCCTTCAAAGTACCAGGT′] |
| 3930 | NM_000523.3(HOXD13): | 3239 | HOXD13 | [′GAGAGACAAGTGACCATTTGGTTTCRGAA |
| | c.974A > G (p.Gln325Arg) | | | CCGAAGAGTGAAGGACAAGAAA′] |
| 3931 | NM_000545.6(HNF1A): c.365A > G | 6927 | HNF1A | [′CGTGTGGCGAAGATGGTCAAGTCCTRCCT |
| | (p.Tyr122Cys) | | | GCAGCAGCACAACATCCCACAG′] |
| 3932 | NM_000519.3(HBD): c.−81A > G | 3045 | HBD | [′CTGGAGCAGGGAGGACAGGACCAGCRTAA |
| | | | | AAGGCAGGGCAGAGTCGACTGT′] |
| 3933 | NM_000518.4(HBB): c.199A > G | 3043 | HBB | [′CCCTAAGGTGAAGGCTCATGGCAAGRAAG |
| | (p.Lys67Glu) | | | TGCTCGGTGCCTTTAGTGATGG′] |
| 3934 | NM_000518.4(HBB): c.59A > G | 3043 | HBB | [′GTTACTGCCCTGTGGGGCAAGGTGARCGT |
| | (p.Asn20Ser) | | | GGATGAAGTTGGTGGTGAGGCC′] |
| 3935 | NM_000518.4(HBB): c.−81A > G | 3043 | HBB | [′GGGAGGGCAGGAGCCAGGGCTGGGCVTA |
| | | | | AAAGTCAGGGCAGAGCCATCTAT′] |
| 3936 | NM_000518.4(HBB): | 3043 | HBB | [′GAGGGCAGGAGCCAGGGCTGGGCATRAAA |
| | c.−50 − 29A > G | | | GTCAGGGCAGAGCCATCTATTG′] |
| 3937 | NM_000518.4(HBB): c.−78A > G | 3043 | HBB | [′AGGGCAGGAGCCAGGGCTGGGCATAVAA |
| | | | | GTCAGGGCAGAGCCATCTATTGC′] |
| 3938 | NM_000518.4(HBB): c.*113A > G | 3043 | HBB | [′TGAGCATCTGGATTCTGCCTAATAARAAAC |
| | | | | ATTTATTTTCATTGCAATGAT′] |
| 3939 | NM_000518.4(HBB): c.*112A > G | 3043 | HBB | [′TTGAGCATCTGGATTCTGCCTAATADAAAA |
| | | | | CATTTATTTTCATTGCAATGA′] |
| 3940 | NM_000518.4(HBB): c.*111A > G | 3043 | HBB | [′CTTGAGCATCTGGATTCTGCCTAATRAAAA |
| | | | | ACATTTATTTTCATTGCAATG′] |
| 3941 | NM_000518.4(HBB): c.80A > G | 3043 | HBB | [′GTGAACGTGGATGAAGTTGGTGGTGNGGC |
| | (p.Glu27Gly) | | | CCTGGGCAGGTTGGTATCAAGG′] |
| 3942 | NM_000518.4(HBB): c.247A > G | 3043 | HBB | [′TGGCCTGGCTCACCTGGACAACCTCVAGG |
| | (p.Lys83Glu) | | | GCACCTTTGCCACACTGAGTGA′] |
| 3943 | NM_000517.4(HBA2): c.1A > G | 3040 | HBA2 | [′CCCACAGACTCAGAGAGAACCCACCRTGG |
| | (p.Met1Val) | | | TGCTGTCTCCTGCCGACAAGAC′] |
| 3944 | NM_000517.4(HBA2): c.*92A > G | 3040 | HBA2 | [′CACCGGCCCTTCCTGGTCTTTGAATRAAGT |
| | | | | CTGAGTGGGCAGCAGCCTGTG′] |
| 3945 | NM_000517.4(HBA2): | 3040 | HBA2 | [′CACCCCTCACTCTGCTTCTCCCCGCRGGAT |
| | c.96 − 2A > G | | | GTTCCTGTCCTTCCCCACCAC′] |
| 3946 | NM_006121.3(KRT1): c.1445A > G | 3848 | KRT1 | [′GCCCTGGATCTGGAGATTGCCACCTRCAG |
| | (p.Tyr482Cys) | | | GACCCTCCTGGAGGGAGAAGAA′] |
| 3947 | NM_001077488.3(GNAS): c.1A > G | 2778 | GNAS | [′GCGCCCCGCCGCCGCCGCCGCCGCCRTGG |
| | (p.Met1Val) | | | GCTGCCTCGGGAACAGTAAGAC′] |
| 3948 | NM_000516.5(GNAS): c.680A > G | 2778 | GNAS | [′ACCAGCATGTTTGACGTGGGTGGCCDGCG |
| | (p.Gln227Arg) | | | CGATGAACGCCGCAAGTGGATC′] |
| 3949 | NM_000515.4(GH1): c.413A > G | 2688 | GH1 | [′GGCGCCTCTGACAGCAACGTCTATGRCCTC |
| | (p.Asp138Gly) | | | CTAAAGGACCTAGAGGAAGGC′] |
| 3950 | NM_000823.3(GHRHR): c.985A > G | 2692 | GHRHR | [′TTGTCTTTCCTGCAGGCGTCTCTCCRAGTC |
| | (p.Lys329Glu) | | | GACACTTTTCCTGATCCCACT′] |
| 3951 | NM_002890.2(RASA1): | 5921 | RASA1 | [′GACCAATGAAAATATTCAGCGATTTRAAA |
| | c.1198A > G (p.Lys400Glu) | | | TATGTCCAACGCCAAACAATCA′] |
| 3952 | NM_002890.2(RASA1): | 5921 | RASA1 | [′CAATGAAAATATTCAGCGATTTAAARTAT |
| | c.1201A > G (p.Ile401Val) | | | GTCCAACGCCAAACAATCAGTT′] |
| 3953 | NM_000406.2(GNRHR): c.317A > G | 2798 | GNRHR | [′GATGGGATGTGGAACATTACAGTCCRATG |
| | (p.Gln106Arg) | | | GTATGCTGGAGAGTTACTCTGC′] |
| 3954 | NM_000406.2(GNRHR): c.851A > G | 2798 | GNRHR | [′TTTACTGTCTGCTGGACTCCCTACTRTGTC |
| | (p.Tyr284Cys) | | | CTAGGAATTTGGTATTGGTTT′] |
| 3955 | NM_000407.4(GP1BB): c.338A > G | −1 | — | [′GCCGGCCGCCCCGAGCGTGCGCCCTDCCG |
| | (p.Tyr113Cys) | | | CGACCTGCGTTGCGTGGCGCCC′] |
| 3956 | NM_021957.3(GYS2): c.116A > G | 2998 | GYS2 | [′TTTGAAGTTGCTTGGGAAGTGACCARTAA |
| | (p.Asn39Ser) | | | AGGTTTGTACTGCTCCTGAAGG′] |
| 3957 | NM_001146040.1(GLRA1): | 2741 | GLRA1 | [′CCTCCACCCCCACTCTAGGTGTCCTVTGTG |
| | c.920A > G (p.Tyr307Cys) | | | AAAGCCATTGACATTTGGATG′] |
| 3958 | NM_001146040.1(GLRA1): | 2741 | GLRA1 | [′CTCCGGCTCTCGAGCATCTCTGCCCRAGGT |
| | c.910A > G (p.Lys304Glu) | | | AAGTCCCATTGCCCAAGAGCA′] |
| 3959 | NM_000171.3(GLRA1): c.523A > G | 2741 | GLRA1 | [′CCCCATGGACTTGAAGAATTTCCCCRTGGA |
| | (p.Met175Val) | | | TGTCCAGACATGTATCATGCA′] |
| 3960 | NM_000162.3(GCK): c.641A > G | 2645 | GCK | [′ACGGTGGCCACGATGATCTCCTGCTRCTAC |
| | (p.Tyr214Cys) | | | GAAGACCATCAGTGCGAGGTC′] |
| 3961 | NM_000145.3(FSHR): c.1345A > G | 2492 | FSHR | [′AGGCTGTGATGCTGCTGGCTTTTTCRCTGT |
| | (p.Thr449Ala) | | | CTTTGCCAGTGAGCTGTCAGT′] |
| 3962 | NM_182925.4(FLT4): c.3104A > G | 2324 | FLT4 | [′CGCCTCCCCGCACCCCAGTGCATCCRCAGA |
| | (p.His 1035Arg) | | | GACCTGGCTGCTCGGAACATT′] |
| 3963 | NM_023110.2(FGFR1): | 2260 | FGFR1 | [′CCGGCAGTGATGACCTCGCCCCTGTRCCTG |
| | c.1121A > G (p.Tyr374Cys) | | | GAGATCATCATCTATTGCACA′] |
| 3964 | NM_006894.5(FMO3): c.182A > G | 2328 | FMO3 | [′AGCATTTACAAATCAGTCTTTTCCARCTCT |
| | (p.Asn61Ser) | | | TCCAAAGAGATGATGTGTTTC′] |
| 3965 | NM_001002294.2(FMO3): | 2328 | FMO3 | [′AAGCCTAACGTGAAGGAATTCACAGRGAC |
| | c.923A > G (p.Glu308Gly) | | | CTCGGCCATTTTTGAGGATGGG′] |
| 3966 | NM_212482.1(FN1): c.2918A > G | 2335 | FN1 | [′ACCGGGCTGTCCCCTGGGGTCACCTRTTAC |
| | (p.Tyr973Cys) | | | TTCAAAGTCTTTGCAGTGAGC′] |
| 3967 | NM_000142.4(FGFR3): | 2261 | FGFR3 | [′CGTGCACAACCTCGACTACTACAAGVAGA |
| | c.1948A > G (p.Lys650Glu) | | | CAACCAACGTGAGCCCGGCCCT′] |
| 3968 | NM_000142.4(FGFR3): c.1118A > G | 2261 | FGFR3 | [′GAGGCTGACGAGGCGGGCAGTGTGTRTGC |
| | (p.Tyr373Cys) | | | AGGCATCCTCAGCTACGGGGTG′] |
| 3969 | NM_000142.4(FGFR3): c.1612A > G | 2261 | FGFR3 | [′GTGCAGGCGCCCAGCAGGTTGATGAYGTT |
| | (p.Ile538Val) | | | TTTGTGTTTCCCGATCATCTTC′] |
| 3970 | NM_000142.4(FGFR3): c.1619A > G | 2261 | FGFR3 | [′GCCCTGCGTGCAGGCGCCCAGCAGGBTGA |
| | (p.Asn540Ser) | | | TGATGTTTTTGTGTTTCCCGAT′] |
| 3971 | NM_000142.4(FGFR3): c.833A > G | 2261 | FGFR3 | [′GACGTGGAGTTCCACTGCAAGGTGTRCAG |
| | (p.Tyr278Cys) | | | TGACGCACAGCCCCACATCCAG′] |
| 3972 | NM_000138.4(FBN1): c.6431A > G | 2200 | FBN1 | [′GTCTGTAAACATGGACAGTGCATCARTAC |
| | (p.Asn2144Ser) | | | AGATGGTTCCTATCGCTGCGAG′] |
| 3973 | NM_000138.4(FBN1): c.3128A > G | 2200 | FBN1 | [′ATACCCAGCCTCTGCACCCACGGCARGTG |
| | (p.Lys1043Arg) | | | CAGAAACACCATTGGCAGCTTT′] |
| 3974 | NM_000138.4(FBN1): c.2261A > G | 2200 | FBN1 | [′TATAAATGTATATGCAATTCAGGATRTGAA |
| | (p.Tyr754Cys) | | | GTGGATTCAACTGGGAAAAAC′] |
| 3975 | NM_000146.3(FTL): c.−160A > G | 2512 | FTL | [′CCGCGGGTCTGTCTCTTGCTTCAACRGTGT |
| | | | | TTGGACGGAACAGATCCGGGG′] |
| 3976 | NM_000043.4(FAS): c.695A > G | 355 | FAS | [′TTTTCAGATGTTGACTTGAGTAAATRTATC |
| | (p.Tyr232Cys) | | | ACCACTATTGCTGGAGTCATG′] |
| 3977 | NM_000043.4(FAS): c.763A > G | 355 | FAS | [′AGGCTTTGTTCGAAAGAATGGTGTCRATG |
| | (p.Asn255Asp) | | | AAGCCAAAATAGATGAGATCAA′] |
| 3978 | NM_000043.4(FAS): c.353A > G | 355 | FAS | [′TTTCTAGGCTTAGAAGTGGAAATAARCTGC |
| | (p.Asn118Ser) | | | ACCCGGACCCAGAATACCAAG′] |
| 3979 | NM_000129.3(F13A1): c.851A > G | 2162 | F13A1 | [′CTCGTTGGATCCTGGGACAATATCTRTGCC |
| | (p.Tyr284Cys) | | | TATGGCGTCCCCCCATCGGCC′] |
| 3980 | NM_000121.3(EPOR): c.1460A > G | 2057 | EPOR | [′GGCTTATCCGATGGCCCCTACTCCARCCCT |
| | (p.Asn487Ser) | | | TATGAGAACAGCCTTATCCCA′] |
| 3981 | NM_207034.2(EDN3): c.335A > G | 1908 | EDN3 | [′GACAAGGAGTGTGTCTACTATTGCCRCCTG |
| | (p.His112Arg) | | | GACATCATTTGGATCAACACT′] |
| 3982 | NM_001257988.1(TYMP): | 1890 | TYMP | [′CAGAGCGGACAGCCCCTCCACGAGTYTCT |
| | c.665A > G (p.Lys222Arg) | | | TACTGAGAATGGAGGCTTTGGG′] |
| 3983 | NM_003286.2(TOP1): c.1598A > G | −1 | — | [′GTAGAGTTTGACTTCCTCGGGAAGGRCTCC |
| | (p.Asp533Gly) | | | ATCAGATACTATAACAAGGTC′] |
| 3984 | NM_001943.3(DSG2): c.797A > G | 1829 | DSG2 | [′GTTCAGATTCGTATTTTGGATGTCARTGAC |
| | (p.Asn266Ser) | | | AATATACCTGTAGTAGAAAAT′] |
| 3985 | NM_001943.3(DSG2): | 1829 | DSG2 | [′TCTGTGTTCAATTTTGTGTCTGTACRGTGG |
| | c.1880 − 2A > G | | | TACCACTTTTACTGCTGATGT′] |
| 3986 | NM_024422.4(DSC2): | 1824 | DSC2 | [′TTTCTCCTTATTTTACTCTCTTCACRGATAA |
| | c.631 − 2A > G | | | TTGCCTTTGCAACAACTCCA′] |
| 3987 | NG_012088.1: g.2209A > G | 3586 | IL10 | [′CCAGGATGGTCTCGATCTCCTGACCYTATG |
| | | | | ATCCGCCCGCCTTGGCCTCCC′] |
| 3988 | NM_000498.3(CYP11B2): | −1 | — | [′GAGGCCTGGCACGTCCCCCCTCCTCRCTTT |
| | c.1492A > G (p.Thr498Ala) | | | CAGAGCGATTAACTAGTCTTG′] |
| 3989 | NM_000941.2(POR): c.1733A > G | 5447 | POR | [′CGCCGCTCGGATGAGGACTACCTGTRCCG |
| | (p.Tyr578Cys) | | | GGAGGAGCTGGCGCAGTTCCAC′] |
| 3990 | NM_000941.2(POR): c.15A > G | 5447 | POR | [′CTAACAGTTTCATGATCAACATGGGRGACT |
| | (p.Gly5=) | | | CCCACGTGGACACCAGCTCCA′] |
| 3991 | NM_001885.2(CRYAB): c.358A > G | 1410 | CRYAB | [′TGGTTTCATCTCCAGGGAGTTCCACRGGAA |
| | (p.Arg120Gly) | | | ATACCGGATCCCAGCTGATGT′] |
| 3992 | NM_021954.3(GJA3): c.188A > G | 2700 | GJA3 | [′AACACCCAGCAGCCGGGCTGCGAGARCGT |
| | (p.Asn63Ser) | | | CTGCTACGACAGGGCCTTCCCC′] |
| 3993 | NM_004004.5(GJB2): c.218A > G | 2706 | GJB2 | [′TACGATCACTACTTCCCCATCTCCCRCATC |
| | (p.His73Arg) | | | CGGCTATGGGCCCTGCAGCTG′] |
| 3994 | NM_001735.2(C5): c.1115A > G | 727 | C5 | [′CGTCTACCCCCTCACCCAATCTACCYTGAT |
| | (p.Lys372Arg) | | | GGGATATGGAATCCCAGGCTT′] |
| 3995 | NM_001848.2(COL6A1): | 1291 | COL6A1 | [′CTCAAAAGCAGCGTGGACGCGGTCARGTA |
| | c.362A > G (p.Lys121Arg) | | | CTTTGGGAAGGGCACCTACACC′] |
| 3996 | NM_000093.4(COL5A1): | 1289 | COL5A1 | [′TTTTCATGAGCGTCTCTTCTTTTCCRGGGTG |
| | c.655 − 2A > G | | | ACATCCAGCAGCTGCTCTTT′] |
| 3997 | NM_000089.3(COL1A2): | 1278 | COL1A2 | [′TATAATTTTTTTTTTTTACTTCTCTRGAACT |
| | c.226 − 2A > G | | | TTGCTGCTCAGTATGATGGA′] |
| 3998 | NM_000089.3(COL1A2): | 1278 | COL1A2 | [′GAGTGGGTACATTCTGAAAAGTAATRTAA |
| | c.70 + 717A > G | | | GTGTCTCAATTCACTTTCTAGT′] |
| 3999 | NM_001844.4(COL2A1): | 1280 | COL2A1 | [′ACGGAAGGCTCCCAGAACATCACCTRCCA |
| | c.4172A > G (p.Tyr1391Cys) | | | CTGCAAGAACAGCATTGCCTAT′] |
| 4000 | NM_001844.4(COL2A1): | 1280 | COL2A1 | [′CGGTCTGCCTGGGCAACGTGGTGAGRGAG |
| | c.2974A > G (p.Arg992Gly) | | | GATTCCCTGGCTTGCCTGGCCC′] |
| 4001 | NM_000493.3(COL10A1): | −1 | — | [′TTTACTTGTCAGATACCAGGAATATRCTAT |
| | c.1790A > G (p.Tyr597Cys) | | | TTTTCATACCACGTGCATGTG′] |
| 4002 | NM_004385.4(VCAN): | 1462 | VCAN | [′GTATTGTGAAAACTCTGTTTTTTTCDGGTC |
| | c.4004 − 2A > G | | | GAATGAGTGATTTGAGTGTAA′] |
| 4003 | NM_020549.4(CHAT): c.1444A > G | 1103 | CHAT | [′CGTCAGCGAGCTCCCCGCCCCCCGGVGGC |
| | (p.Arg482Gly) | | | TGCGGTGGAAATGCTCCCCGGA′] |
| 4004 | NM_000078.2(CETP): c.1376A > G | 1071 | CETP | [′AACAGCAAAGGCGTGAGCCTCTTCGRCAT |
| | (p.Asp459Gly) | | | CATCAACCCTGAGATTATCACT′] |
| 4005 | NM_000083.2(CLCN1): | 1180 | CLCN1 | [′GTGATTTGCTTCGAATTAACGGGTCDGATT |
| | c.1655A > G (p.Gln552Arg) | | | GCTCACATCCTGCCCATGATG′] |
| 4006 | NM_000083.2(CLCN1): c.382A > G | 1180 | CLCN1 | [′CTTTCTGGTGCTTCTGGGACTGCTGRTGGC |
| | (p.Met128Val) | | | TCTGGTCAGCTGGAGCATGGA′] |
| 4007 | NM_199440.1(HSPD1): c.86A > G | 3329 | HSPD1 | [′CATCTCACTCGGGCTTATGCCAAAGRTGTA |
| | (p.Asp29Gly) | | | AAATTTGGTGCAGATGCCCGA′] |
| 4008 | NM_001904.3(CTNNB1): c.95A > G | 1499 | CTNNB1 | [′CACTGGCAGCAACAGTCTTACCTGGNCTCT |
| | (p.Asp32Gly) | | | GGAATCCATTCTGGTGCCACT′] |
| 4009 | NM_001904.3(CTNNB1): | 1499 | CTNNB1 | [′CTCTGGAATCCATTCTGGTGCCACTNCCAC |
| | c.121A > G (p.Thr41Ala) | | | AGCTCCTTCTCTGAGTGGTAA′] |
| 4010 | NM_007294.3(BRCA1): c.211A > G | 672 | BRCA1 | [′TTTATGTAAGAATGATATAACCAAARGGT |
| | (p.Arg71Gly) | | | ATATAATTTGGTAATGATGCTA′] |
| 4011 | NM_001202.3(BMP4): c.278A > G | 652 | BMP4 | [′GATCTTTACCGGCTTCAGTCTGGGGRGGAG |
| | (p.Glu93Gly) | | | GAGGAAGAGCAGATCCACAGC′] |
| 4012 | NM_000342.3(SLC4A1): | 6521 | SLC4A1 | [′CACCACATCACACCCGGGTACCCACRAGG |
| | c.166A > G (p.Lys56Glu) | | | TGAGGACCCCAGCCTCCTCCGT′] |
| 4013 | NM_000342.3(SLC4A1): | 6521 | SLC4A1 | [′GAAGACCTGGCGCATGCACTTATTCRCGG |
| | c.2509A > G (p.Thr837Ala) | | | GCATCCAGATCATCTGCCTGGC′] |
| 4014 | NM_001681.3(ATP2A2): | 488 | ATP2A2 | [′TTCATCCGCTACCTCATCTCGTCCARCGTC |
| | c.2300A > G (p.Asn767Ser) | | | GGGGAAGTTGTCTGGTAGGTC′] |
| 4015 | NM_000486.5(AQP2): c.203A > G | 359 | AQP2 | [′GGCCACATAAGCGGGGCCCACATCAVCCC |
| | (p.Asn68Ser) | | | TGCCGTGACTGTGGCCTGCCTG′] |
| 4016 | NM_000041.3(APOE): | 348 | APOE | [′GACACCCTCCCGCCCTCTCGGCCGCRGGGC |
| | c.237 − 2A > G | | | GCTGATGGACGAGACCATGAA′] |
| 4017 | NM_000041.3(APOE): c.490A > G | 348 | APOE | [′CGCCTCCCACCTGCGCAAGCTGCGTVAGC |
| | (p.Lys164Glu) | | | GGCTCCTCCGCGATGCCGATGA′] |
| 4018 | NM_000041.3(APOE): c.178A > G | 348 | APOE | [′TTGGGATTACCTGCGCTGGGTGCAGRCACT |
| | (p.Thr60Ala) | | | GTCTGAGCAGGTGCAGGAGGA′] |
| 4019 | NM_000040.1(APOC3): c.280A > G | 345 | APOC3 | [′GGATTTGGACCCTGAGGTCAGACCARCTTC |
| | (p.Thr94Ala) | | | AGCCGTGGCTGCCTGAGACCT′] |
| 4020 | NM_000040.1(APOC3): c.232A > G | 345 | APOC3 | [′CCTGAAAGACTACTGGAGCACCGTTRAGG |
| | (p.Lys78Glu) | | | ACAAGTTCTCTGAGTTCTGGGA′] |
| 4021 | NM_001042425.1(TFAP2A): | 7020 | TFAP2A | [′GTCGCTGCTGGGCGGAGTGCTCCGGVGGT |
| | c.751A > G (p.Arg251Gly) | | | GAGGCCCGGCACGGCCCCGCCC′] |
| 4022 | NM_000488.3(SERPINC1): | 462 | SERPINC1 | [′TGCAGAGCAATCCAGAGCGGCCATCRACA |
| | c.655A > G (p.Asn219Asp) | | | AATGGGTGTCCAATAAGACCGA′] |
| 4023 | NM_001085.4(SERPINA3): | 12 | SERPINA3 | [′TACAGACACCCAGAACATCTTCTTCRTGAG |
| | c.1240A > G (p.Met414Val) | | | CAAAGTCACCAATCCCAAGCA′] |
| 4024 | NM_001148.4(ANK2): c.4373A > G | 287 | ANK2 | [′TGCATGGCATCTTGGGGCGGAAAGGRATC |
| | (p.Glu1458Gly) | | | AGAGTCAGATCAAGAACAGGAG′] |
| 4025 | NM_001145.4(ANG): c.121A > G | −1 | — | [′CTTCCTGACCCAGCACTATGATGCCRAACC |
| | (p.Lys41Glu) | | | ACAGGGCCGGGATGACAGATA′] |
| 4026 | NM_001145.4(ANG): c.208A > G | −1 | — | [′ACCCTGCAAAGACATCAACACATTTRTTCA |
| | (p.Ile70Val) | | | TGGCAACAAGCGCAGCATCAA′] |
| 4027 | NM_000484.3(APP): c.2146A > G | 351 | APP | [′GGGCGGTGTTGTCATAGCGACAGTGRTCG |
| | (p.Ile716Val) | | | TCATCACCTTGGTGATGCTGAA′] |
| 4028 | NM_000484.3(APP): c.2078A > G | 351 | APP | [′TTTGTTTTCAAGGTGTTCTTTGCAGRAGAT |
| | (p.Glu693Gly) | | | GTGGGTTCAAACAAAGGTGCA′] |
| 4029 | NM_000484.3(APP): c.2140A > G | 351 | APP | [′CATGGTGGGCGGTGTTGTCATAGCGRCAG |
| | (p.Thr714Ala) | | | TGATCGTCATCACCTTGGTGAT′] |
| 4030 | NM_000021.3(PSEN1): c.488A > G | 5663 | PSEN1 | [′AAATGCTTTCTTTTCTAGGTCATCCRTGCC |
| | (p.His163Arg) | | | TGGCTTATTATATCATCTCTA′] |
| 4031 | NM_000021.3(PSEN1): c.415A > G | 5663 | PSEN1 | [′GCACTCAATTCTGAATGCTGCCATCRTGAT |
| | (p.Met139Val) | | | CAGTGTCATTGTTGTCATGAC′] |
| 4032 | NM_000021.3(PSEN1): c.436A > G | 5663 | PSEN1 | [′CATCATGATCAGTGTCATTGTTGTCNTGAC |
| | (p.Met146Val) | | | TATCCTCCTGGTGGTTCTGTA′] |
| 4033 | NM_000021.3(PSEN1): c.839A > G | 5663 | PSEN1 | [′GTTGAAACAGCTCAGGAGAGAAATGVAAC |
| | (p.Glu280Gly) | | | GCTTTTTCCAGCTCTCATTTAC′] |
| 4034 | NM_000021.3(PSEN1): c.998A > G | 5663 | PSEN1 | [′CAAGACACTGTTGCAGAGAATGATGRTGG |
| | (p.Asp333Gly) | | | CGGGTTCAGTGAGGAATGGGAA′] |
| 4035 | NM_000034.3(ALDOA): c.386A > G | 226 | ALDOA | [′CCCCTTCCTCTTCTCTTAGGGTTGGRTGGG |
| | (p.Asp129Gly) | | | CTGTCTGAGCGCTGTGCCCAG′] |
| 4036 | NM_000477.5(ALB): | 213 | ALB | [′CATTTTGATTGGCGATTTTCTTTTTDGGGC |
| | c.714 − 2A > G | | | AGTAGCTCGCCTGAGCCAGAG′] |
| 4037 | NM_001151.3(SLC25A4): | 291 | SLC25A4 | [′AAGCAGCTCTTCTTAGGGGGTGTGGRTCG |
| | c.311A > G (p.Asp104Gly) | | | GCATAAGCAGTTCTGGCGCTAC′] |
| 4038 | NM_013411.4(AK2): c.1A > G | 204 | AK2 | [′GTGGCAGTGAGAGACTTCGGCGGACRTGG |
| | (p.Met1Val) | | | CTCCCAGCGTGCCAGCGGCAGA′] |
| 4039 | NM_013411.4(AK2): c.494A > G | 204 | AK2 | [′AACCCTCCAAAAGAGCCCATGAAAGRTGA |
| | (p.Asp165Gly) | | | CGTATGTAAACTCAGGACAAAA′] |
| 4040 | NM_000476.2(AK1): c.491A > G | 203 | AK1 | [′GCCACAGAACCCGTCATCGCCTTCTRTGAG |
| | (p.Tyr164Cys) | | | AAACGTGGCATTGTGCGCAAG′] |
| 4041 | NM_001100.3(ACTA1): c.350A > G | 58 | ACTA1 | [′GAGGCCCCCCTCAATCCCAAGGCCARCCG |
| | (p.Asn117Ser) | | | CGAGAAGATGACCCAGATCATG′] |
| 4042 | NM_001103.3(ACTN2): c.26A > G | 88 | ACTN2 | [′ATGAACCAGATAGAGCCCGGCGTGCRGTA |
| | (p.Gln9Arg) | | | CAACTACGTGTACGACGAGGAT′] |
| 4043 | NM_005159.4(ACTC1): | −1 | — | [′CAGCAAATGTGGATTAGCAAGCAAGRGTA |
| | c.1088A > G (p.Glu363Gly) | | | CGATGAGGCAGGCCCATCCATT′] |
| 4044 | NM_005159.4(ACTC1): c.373A > G | −1 | — | [′CAACCGGGAGAAGATGACTCAGATCRTGT |
| | (p.Met125Val) | | | TTGAGACCTTCAATGTCCCTGC′] |
| 4045 | NM_018109.3(MTPAP): | 55149 | MTPAP | [′TGATTCTTCTCCTCTGTACATTCAGRATCC |
| | c.1432A > G (p.Asn478Asp) | | | ATTTGAAACTTCTCTCAACAT′] |
| 4046 | NM_016955.3(SEPSECS): | 51091 | SEPSECS | [′TTATTGTCACTTGGATCAAATGGCTRTAAG |
| | c.1001A > G (p.Tyr334Cys) | | | AAGCTACTAAAAGAAAGAAAG′] |
| 4047 | NM_014053.3(FLVCR1): | 28982 | FLVCR1 | [′GATCTTCAGCCTGTACTCGCTGGTCRACGC |
| | c.361A > G (p.Asn121Asp) | | | CTTTCAGTGGATCCAGTACAG′] |
| 4048 | NM_000021.3(PSEN1): c.697A > G | 5663 | PSEN1 | [′ATATCTCATTATGATTAGTGCCCTCDTGGC |
| | (p.Met233Val) | | | CCTGGTGTTTATCAAGTACCT′] |
| 4049 | NM_000207.2(INS): c.323A > G | −1 | — | [′TGCTCCCTCTACCAGCTGGAGAACTRCTGC |
| | (p.Tyr108Cys) | | | AACTAGACGCAGCCCGCAGGC′] |
| 4050 | NM_000334.4(SCN4A): | 6329 | SCN4A | [′GAAGCAGGCCTTCGACATCACCATCRTGA |
| | c.4078A > G (p.Met1360Val) | | | TCCTCATCTGCCTCAACATGGT′] |
| 4051 | NM_000334.4(SCN4A): | 6329 | SCN4A | [′CCTCATCTGCCTCAACATGGTCACCRTGAT |
| | c.4108A > G (p.Met1370Val) | | | GGTGGAGACAGACAACCAGAG′] |
| 4052 | NM_000518.4(HBB): | 3043 | HBB | [′TTCATACCTCTTATCTTCCTCCCACVGCTCC |
| | c.316 − 2A > G | | | TGGGCAACGTGCTGGTCTGT′] |
| 4053 | NM_000525.3(KCNJ11): | 3767 | KCNJ11 | [′GTGGCCCACAAGAACATCCGGGAGCRGGG |
| | c.155A > G (p.Gln52Arg) | | | CCGCTTCCTGCAGGACGTGTTC′] |
| 4054 | NM_000525.3(KCNJ11): | 3767 | KCNJ11 | [′AGCCCACCGCAGGGCTGAGACCCTCRTCTT |
| | c.544A > G (p.Ile182Val) | | | CAGCAAGCATGCGGTGATCGC′] |
| 4055 | NM_000525.3(KCNJ11): | 3767 | KCNJ11 | [′GGAAGGCGTGGTGGAAACCACGGGCVTCA |
| | c.886A > G (p.Ile296Val) | | | CCACCCAGGCCCGCACCTCCTA′] |
| 4056 | NM_000525.3(KCNJ11): | 3767 | KCNJ11 | [′GAGGACGGACGTTACTCTGTGGACTRCTCC |
| | c.989A > G (p.Tyr330Cys) | | | AAGTTTGGCAACACCGTCAAA′] |
| 4057 | NM_000528.3(MAN2B1): | 4125 | MAN2B1 | [′CCCATCTGTGGACCCTTTTCTGCCCRGCAC |
| | c.1831 − 2A > G | | | ATCCGGGCAACGTTTGATCCT′] |
| 4058 | NM_000639.2(FASLG): c.466A > G | 356 | FASLG | [′ATTTATTTCAGGCAAGTCCAACTCARGGTC |
| | (p.Arg156Gly) | | | CATGCCTCTGGAATGGGAAGA′] |
| 4059 | NM_000920.3(PC): c.1705A > G | 5091 | PC | [′CCCGGGGCTGCTGCTGATGGACACGRCCTT |
| | (p.Thr569Ala) | | | CAGGGACGCCCACCAGTCACT′] |
| 4060 | NM_001017420.2(ESCO2): | 157570 | ESCO2 | [′AATGGGTTTCTTTTTTTACCCCCCARTTATA |
| | c.1132 − 7A > G | | | GGACGCTGGTCAGAAACATT′] |
| 4061 | NM_001017420.2(ESCO2): | 157570 | ESCO2 | [′ATTAAATCATCTTTTCTTCTCTTTTRGGAAT |
| | 2c.1674 − A > G | | | TGCTTCATGTTTGGCTGTTT′] |
| 4062 | NM_001129765.1(NSDHL): | 50814 | NSDHL | [′GAGAGAGCCAAAAAGGCCATGGGCTVCCA |
| | c.1046A > G (p.Tyr349Cys) | | | GCCACTAGTGACCATGGATGAT′] |
| 4063 | NM_001701.3(BAAT): c.967A > G | 570 | BAAT | [′AGAGGCCCAGGGGCAATTCCTCTTCRTTGT |
| | (p.Ile323Val) | | | AGGAGAAGGTGATAAGACTAT′] |
| 4064 | NM_002977.3(SCN9A): c.406A > G | 6335 | SCN9A | [′ATTCAGCATGCTCATCATGTGCACTRTTCT |
| | (p.Ile136Val) | | | GACAAACTGCATATTTATGAC′] |
| 4065 | NM_004004.5(GJB2): c.487A > G | 2706 | GJB2 | [′CTATGTCATGTACGACGGCTTCTCCRTGCA |
| | (p.Met163Val) | | | GCGGCTGGTGAAGTGCAACGC′] |
| 4066 | NM_004519.3(KCNQ3): | 3786 | KCNQ3 | [′GAACCAAAGCCTGTTGGCTTAAACARTAA |
| | c.1403A > G (p.Asn468Ser) | | | AGAGCGTTTCCGCACGGCCTTC′] |
| 4067 | NM_004519.3(KCNQ3): | 3786 | KCNQ3 | [′AGAGATGATTATGTGTTCGGCCCCARTGG |
| | c.2462A > G (p.Asn821Ser) | | | GGGGTCGAGCTGGATGAGGGAG′] |
| 4068 | NM_004519.3(KCNQ3): c.914A > G | 3786 | KCNQ3 | [′AAAGAGGAGTTTGAGACCTATGCAGRTGC |
| | (p.Asp305Gly) | | | CCTGTGGTGGGGCCTGGTGAGT′] |
| 4069 | NM_004525.2(LRP2): | 4036 | LRP2 | [′TGTAACTCTCTCTTTTTCCCCCCACRGAAA |
| | c.770 − 2A > G | | | GCGGTCCTCATGATGTTCATA′] |
| 4070 | NM_007375.3(TARDBP): c.1055A | 23435 | TARDBP | [′CAGAACCAGTCAGGCCCATCGGGTARTAA |
| | > G (p.Asn352Ser) | | | CCAAAACCAAGGCAACATGCAG′] |
| 4071 | NM_007375.3(TARDBP): c.800A > | 23435 | TARDBP | [′AATGCCGAACCTAAGCACAATAGCARTAG |
| | G (p.Asn267Ser) | | | ACAGTTAGAAAGAAGTGGAAGA′] |
| 4072 | NM_012434.4(SLC17A5): c.406A > | 26503 | SLC17A5 | [′ATATGTTGCCAGCAAAATAGGGGGGDAAA |
| | G (p.Lys136Glu) | | | TGCTGCTAGGATTTGGGATCCT′] |
| 4073 | NM_012463.3(ATP6V0A2): | 23545 | ATP6V0A | [′AGACTGTGTTCAACTCTTGTCTTCCRGCTA |
| | c.732 − 2A > G | | 2 | CCACTGCCACGTGTACCCCTA′] |
| 4074 | NM_014043.3(CHMP2B): c.85A > G | 25978 | CHMP2B | [′GTTACGAGGTACACAGAGGGCTATARTCA |
| | (p.Ile29Val) | | | GAGATCGAGCAGCTTTAGAGAA′] |
| 4075 | NM_018136.4(ASPM): | 25926 | ASPM | [′AGCAAAATTATAATATTGGAATATARTATC |
| | c.2761 − 25A > G | 6 | | TGGAACTTATTTCTTTATAGG′] |
| 4076 | NM_022081.5(HPS4): c.461A > G | 89781 | HPS4 | [′ATTCTGAAAAACACCAGTGATCTGCRTAA |
| | (p.His154Arg) | | | GATTTTCAATTCCCTCTGGAAC′] |
| 4077 | NM_024649.4(BBS1): | −1 | — | [′GTCAGCCTCTGGGACCCTTCTCCACRGCCA |
| | c.1340 − 2A > G | | | TGCACCGGGCCTTCCAGACAG′] |
| 4078 | NM_032520.4(GNPTG): | 84572 | GNPTG | [′TGCTGCCCCTGCATCCTCCACCTTCRGGGC |
| | 2c.610 − A > G | | | CATGAGAAGTTGCTGAGGACA′] |
| 4079 | NM_172107.2(KCNQ2): c.1A > G | 3785 | KCNQ2 | [′CCCGGGGCGCCTCCCGCCAGGCACCRTGG |
| | (p.Met1Val) | | | TGCAGAAGTCGCGCAACGGCGG′] |
| 4080 | NM_172107.2(KCNQ2): c.356A > G | 3785 | KCNQ2 | [′GTGTTTTCCACCATCAAGGAGTATGRGAA |
| | (p.Glu119Gly) | | | GAGCTCGGAGGGGGCCCTCTAC′] |
| 4081 | NM_172107.2(KCNQ2): c.622A > G | 3785 | KCNQ2 | [′CCTGCGCTTCCTGCAGATTCTGCGGRTGAT |
| | (p.Met208Val) | | | CCGCATGGACCGGCGGGGAGG′] |
| 4082 | NM_172107.2(KCNQ2): c.635A > G | 3785 | KCNQ2 | [′CAGATTCTGCGGATGATCCGCATGGRCCG |
| | (p.Asp212Gly) | | | GCGGGGAGGCACCTGGAAGCTG′] |
| 4083 | NM_172107.2(KCNQ2): c.773A > G | 3785 | KCNQ2 | [′GTGTACTTGGCAGAGAAGGGGGAGARCGA |
| | (p.Asn258Ser) | | | CCACTTTGACACCTACGCGGAT′] |
| 4084 | NM_172107.2(KCNQ2): | 3785 | KCNQ2 | [′ATGCTTTGGGGTCTCTGTTCCCGGTRGAGT |
| | c.1764 − 2A > G | | | GGACCAGATCGTGGGGCGGGG′] |
| 4085 | NM_000495.4(COL4A5): c.1A > G | 1287 | COL4A5 | [′AAGGAGCTGCGGGAGCCGGAGAAGARTG |
| | (p.Met1Val) | | | AAACTGCGTGGAGTCAGCCTGGC′] |
| 4086 | NM_000495.4(COL4A5): | 1287 | COL4A5 | [′AGAACTTCCATTGATGGCTTCTTTTRGGGT |
| | 2c.466 − A > G | | | GAACCAGGTAGTATAATTATG′] |
| 4087 | NM_000495.4(COL4A5): | 1287 | COL4A5 | [′TCATTTTCTTTACTCACTTTATAACRGGGC |
| | c.547 − 2A > G | | | CTACCTGGTCCCACTGGTATA′] |
| 4088 | NM_000495.4(COL4A5): | 1287 | COL4A5 | [′ATTTTCTCTTTTGTCTTCTCTTCTTRGGGCC |
| | c.610 − 2A > G | | | CTCCTGGTCCACCAGGACTT′] |
| 4089 | NM_000495.4(COL4A5): | 1287 | COL4A5 | [′GCCCTATCATTTCTTTGTATCCTATRGGGT |
| | c.892 − 2A > G | | | AAACCAGGCAAAGATGGAGAA′] |
| 4090 | NM_000495.4(COL4A5): | 1287 | COL4A5 | [′TTGCTATCCTTTCTTTATCTTACTCRGGTGA |
| | c.1340 − 2A > G | | | TGAGATATGTGAACCAGGCC′] |
| 4091 | NM_000495.4(COL4A5): | 1287 | COL4A5 | [′TTCTTTGAACGTTTTCCTTTCAATARCTGCT |
| | c.2042 − 18A > G | | | GTTTCTCCATAGGTGACCCT′] |
| 4092 | NM_000495.4(COL4A5): | 1287 | COL4A5 | [′GTTAAAAAATGACTTATCATTTTACRGGCT |
| | c.2147 − 2A > G | | | TTCCTGGAATTCCAGGACCTC′] |
| 4093 | NM_000495.4(COL4A5): | 1287 | COL4A5 | [′GCTTAGATGGGCTCCCTGGACCAAARGGT |
| | c.2394A > G (p.Lys798=) | | | ATGGAGGCTGTCACTGCATCTC′] |
| 4094 | NM_000495.4(COL4A5): | 1287 | COL4A5 | [′CAGATACATCTTTTAAAACTGCTTCRGTAC |
| | c.2510 − 33A > G | | | TTATTAATATTGATATTGTAT′] |
| 4095 | NM_000495.4(COL4A5): | 1287 | COL4A5 | [′ATTCTTCAAAGGTACCAAAGGTGAARTGG |
| | Gc.2692A > (p.Met898Val) | | | GTATGATGGGACCTCCAGGCCC′] |
| 4096 | NM_000495.4(COL4A5): | 1287 | COL4A5 | [′AGGACCTTTGGGAATTCCTGGCAGGRGTG |
| | c.2746A > G (p.Ser916Gly) | | | GTGTACCTGGTCTTAAAGGTAA′] |
| 4097 | NM_000495.4(COL4A5): | 1287 | COL4A5 | [′ATATTATATATCACATATTTTCAACRGGGC |
| | 2c.3107 − A > G | | | CTCAGGGTGTGGAAGGGCCTC′] |
| 4098 | NM_000495.4(COL4A5): | 1287 | COL4A5 | [′AACTGGGTGTAACCTGCTGTACTCARTTTT |
| | c.3455 − 9A > G | | | TTAGGTGGTGGAGGTCATCCT′] |
| 4099 | NM_000495.4(COL4A5): | 1287 | COL4A5 | [′TCTTCTAATTATACTTTACTTTCATRGGCCA |
| | c.3605 − 2A > G | | | AAAGGGTGATGGAGGATTAC′] |
| 4100 | NM_000495.4(COL4A5): | 1287 | COL4A5 | [′GTAACATTAATGATTTTATTTATTCRGGGT |
| | c.3925 − 2A > G | | | AATCCTGGCCGGCCGGGTCTC′] |
| 4101 | NM_000495.4(COL4A5): | 1287 | COL4A5 | [′ATAAAACTGTATGTACCTTCTGTGCDGGCA |
| | c.3998 − 2A > G | | | TGAAAGGACCCAGTGGAGTAC′] |
| 4102 | NM_000495.4(COL4A5): | 1287 | COL4A5 | [′GGATGGGATTCTCTGTGGATTGGTTRTTCC |
| | Gc.4790A > (p.Tyr1597Cys) | | | TTCATGATGGTATTTTACACT′] |
| 4103 | NM_000495.4(COL4A5): | 1287 | COL4A5 | [′CTGATTGTCTTATTTCTTATTTCCCRGTAAA |
| | c.4977 − 2A > G | | | CCTCAGTCAGAAACGCTGAA′] |
| 4104 | NM_005359.5(SMAD4): | 4089 | SMAD4 | [′TTTCATTTGTTTTCCCCTTTAAACARTTAAG |
| | c.425 − 6A > G | | | ATCTCTCAGGATTAACACTG′] |
| 4105 | NM_005359.5(SMAD4): c.989A > G | 4089 | SMAD4 | [′TATTGGTGTTCCATTGCTTACTTTGRAATG |
| | (p.Glu330Gly) | | | GATGTTCAGGTAGGAGAGACA′] |
| 4106 | NM_020630.4(RET): c.1996A > G | 5979 | RET | [′CCACTGCTACCACAAGTTTGCCCACVAGCC |
| | (p.Lys666Glu) | | | ACCCATCTCCTCAGCTGAGAT′] |
| 4107 | NM_020630.4(RET): c.2342A > G | 5979 | RET | [′CTGTCAGAGTTCAACGTCCTGAAGCRGGTC |
| | (p.Gln781Arg) | | | AACCACCCACATGTCATCAAA′] |
| 4108 | NM_000060.3(BTD): c.194A > G | 686 | BTD | [′TATTATGTGGCTGCCGTGTATGAGCRTCCA |
| | (p.His65Arg) | | | TCCATCCTGAGTCTGAACCCT′] |
| 4109 | NM_000060.3(BTD): c.278A > G | 686 | BTD | [′CTCATGAACCAGAACCTTGACATCTRTGAA |
| | (p.Tyr93Cys) | | | CAGCAAGTGATGACTGCAGCC′] |
| 4110 | NM_000060.3(BTD): c.356A > G | 686 | BTD | [′CCAGAAGATGGCATTCATGGATTCARCTTT |
| | (p.Asn119Ser) | | | ACAAGAACATCCATTTATCCA′] |
| 4111 | NM_000060.3(BTD): c.364A > G | 686 | BTD | [′TGGCATTCATGGATTCAACTTTACARGAAC |
| | (p.Arg122Gly) | | | ATCCATTTATCCATTTTTGGA′] |
| 4112 | NM_000060.3(BTD): c.515A > G | 686 | BTD | [′AGGGGAGATATGTTCTTGGTGGCCARTCTT |
| | (p.Asn172Ser) | | | GGGACAAAGGAGCCTTGTCAT′] |
| 4113 | NM_000060.3(BTD): c.583A > G | 686 | BTD | [′CCCAAAAGATGGGAGATACCAGTTCRACA |
| | (p.Asn195Asp) | | | CAAATGTCGTGTTCAGCAATAA′] |
| 4114 | NM_000060.3(BTD): c.584A > G | 686 | BTD | [′CCAAAAGATGGGAGATACCAGTTCARCAC |
| | (p.Asn195Ser) | | | AAATGTCGTGTTCAGCAATAAT′] |
| 4115 | NM_000060.3(BTD): c.641A > G | 686 | BTD | [′CTTGTTGACCGCTACCGTAAACACARCCTC |
| | (p.Asn214Ser) | | | TACTTTGAGGCAGCATTCGAT′] |
| 4116 | NM_000060.3(BTD): c.880A > G | 686 | BTD | [′GAAAGCTTTTGCTGTTGCCTTTGGCRTCAA |
| | (p.Ile294Val) | | | CGTTCTGGCAGCTAATGTCCA′] |
| 4117 | NM_000060.3(BTD): c.1205A > G | 686 | BTD | [′TTTCACTCTGAGATGATGTATGACARTTTC |
| | (p.Asn402Ser) | | | ACCCTGGTCCCTGTCTGGGGA′] |
| 4118 | NM_000060.3(BTD): c.1313A > G | 686 | BTD | [′AGGCCCACCTTATCCAAAGAGCTGTRTGCC |
| | (p.Tyr438Cys) | | | CTGGGGGTCTTTGATGGGCTT′] |
| 4119 | NM_000060.3(BTD): c.1619A > G | 686 | BTD | [′ACGGCGGCTCTCTATGGGCGCTTGTRTGAG |
| | (p.Tyr540Cys) | | | AGGGACTAGGAAAAGTGTGTG′] |
| 4120 | NM_000155.3(GALT): c.1A > G | 2592 | GALT | [′TTCCAGCGGATCCCCCGGTGGCCTCRTGTC |
| | (p.Met1Val) | | | GCGCAGTGGAACCGATCCTCA′] |
| 4121 | NM_000155.3(GALT): c.67A > G | 2592 | GALT | [′GTCAGAGGCGGACGCCGCAGCAGCARCCT |
| | (p.Thr23Ala) | | | TCCGGGCAAACGGTAACTGCAC′] |
| 4122 | NM_000155.3(GALT): | 2592 | GALT | [′GTGCTTCTAGCCTATCCTTGTCGGTRGGTG |
| | c.253 − 2A > G | | | AATCCCCAGTACGATAGCACC′] |
| 4123 | NM_000155.3(GALT): c.290A > G | 2592 | GALT | [′TACGATAGCACCTTCCTGTTTGACARCGAC |
| | (p.Asn97Ser) | | | TTCCCAGCTCTGCAGCCTGAT′] |
| 4124 | NM_000155.3(GALT): c.308A > G | 2592 | GALT | [′TTTGACAACGACTTCCCAGCTCTGCRGCCT |
| | (p.Gln103Arg) | | | GATGCCCCCAGTCCAGGTAAC′] |
| 4125 | NM_000155.3(GALT): c.379A > G | 2592 | GALT | [′ATCTTTTCTCCCGTCACCACCCAGTRAGGT |
| | (p.Lys127Glu) | | | CATGTGCTTCCACCCCTGGTC′] |
| 4126 | NM_000155.3(GALT): c.424A > G | 2592 | GALT | [′CTGGTCGGATGTAACGCTGCCACTCRTGTC |
| | (p.Met142Val) | | | GGTCCCTGAGATCCGGGCTGT′] |
| 4127 | NM_000155.3(GALT): | 2592 | GALT | [′TTCTTCTCTGCTTTTGCCCCTTGACRGGTAT |
| | c.565 − 2A > G | | | GGGCCAGCAGTTTCCTGCCA′] |
| 4128 | NM_000155.3(GALT): c.574A > G | 2592 | GALT | [′TTTTGCCCCTTGACAGGTATGGGCCRGCAG |
| | (p.Ser192Gly) | | | TTTCCTGCCAGATATTGCCCA′] |
| 4129 | NM_000155.3(GALT): c.626A > G | 2592 | GALT | [′CGTGAGGAGCGATCTCAGCAGGCCTVTAA |
| | (p.Tyr209Cys) | | | GAGTCAGCATGGAGAGCCCCTG′] |
| 4130 | NM_000155.3(GALT): c.752A > G | 2592 | GALT | [′GTCCCCTTCTGGGCAACATGGCCCTVCCAG |
| | (p.Tyr251Cys) | | | ACACTGCTGCTGCCCGTCGGC′] |
| 4131 | NM_000155.3(GALT): c.812A > G | 2592 | GALT | [′CGGCTACCTGAGCTGACCCCTGCTGRGCGT |
| | (p.Glu271Gly) | | | GATGGTCAGTCTCCCAAGTAG′] |
| 4132 | NM_000155.3(GALT): | 2592 | GALT | [′AGGCTGAGAGTCAGGCTCTGATTCCRGAT |
| | c.821 − 2A > G | | | CTAGCCTCCATCATGAAGAAGC′] |
| 4133 | NM_000155.3(GALT): c.854A > G | 2592 | GALT | [′TCCATCATGAAGAAGCTCTTGACCARGTAT |
| | (p.Lys285Arg) | | | GACAACCTCTTTGAGACGTCC′] |
| 4134 | NM_000155.3(GALT): c.968A > G | 2592 | GALT | [′CATTGGCAGCTGCACGCTCATTACTRCCCT |
| | (p.Tyr323Cys) | | | CCGCTCCTGCGCTCTGCCACT′] |
| 4135 | NM_000155.3(GALT): c.1001A > G | 2592 | GALT | [′CTCCTGCGCTCTGCCACTGTCCGGARATTC |
| | (p.Lys334Arg) | | | ATGGTTGGCTACGAAATGCTT′] |
| 4136 | NM_000155.3(GALT): c.1048A > G | 2592 | GALT | [′GCTTGCTCAGGCTCAGAGGGACCTCRCCCC |
| | (p.Thr350Ala) | | | TGAGCAGGTCAGGACTCAGAA′] |
| 4137 | NM_000155.3(GALT): c.1132A > G | 2592 | GALT | [′GCAGAAGGACAGGGAGACAGCAACCRTCG |
| | (p.Ile378Val) | | | CCTGACCACGCCGACCACAGGG′] |
| 4138 | NM_000553.4(WRN): c.403A > G | 7486 | WRN | [′GTTGCTTGAAAATAAAGCAGTTAAARAGG |
| | (p.Lys135Glu) | | | CAGGTGTAGGAATTGAAGGAGA′] |
| 4139 | NM_000433.3(NCF2): c.481A > G | 4688 | NCF2 | [′CTTACCCAGACACACTCCATCGCCTYGTCG |
| | (p.Lys161Glu) | | | ATTTTGGAATGTCTGGGCTCA′] |
| 4140 | NM_198056.2(SCN5A): | 6331 | SCN5A | [′ACAGCGGGGGAGAGCGAGAGCCACCRCAC |
| | c.1673A > G (p.His558Arg) | | | ATCACTGCTGGTGCCCTGGCCC′] |
| 4141 | NM_000074.2(CD40LG): | 959 | CD40LG | [′CAAATTGCGGCACATGTCATAAGTGRGGC |
| | c.386A > G (p.Glu129Gly) | | | CAGCAGTAAAACAACATCTGGT′] |
| 4142 | NM_002863.4(PYGL): c.1016A > G | 5836 | PYGL | [′CCCACCCAGGTGGCCATCCAGCTGARTGA |
| | (p.Asn339Ser) | | | CACTCACCCTGCACTCGCGATC′] |
| 4143 | NM_000142.4(FGFR3): | 2261 | FGFR3 | [′CCCCTTGGGGAGGGCTGCTTCGGCCRGGT |
| | c.1454A > G (p.Gln485Arg) | | | GGTCATGGCGGAGGCCATCGGC′] |
| 4144 | NM_001101.3(ACTB): c.34A > G | 60 | ACTB | [′TGATATCGCCGCGCTCGTCGTCGACVACG |
| | (p.Asn12Asp) | | | GCTCCGGCATGTGCAAGGCCGG′] |
| 4145 | NM_001202.3(BMP4): c.362A > G | 652 | BMP4 | [′TGGGGGAAGAGACTGACCTTCGTGGYGGA |
| | (p.His121Arg) | | | AGCTCCTCACGGTGTTGGCCCG′] |
| 4146 | NM_000094.3(COL7A1): | 1294 | COL7A1 | [′CAGCTGGCCCGACCTGGTGTCCCCARGGT |
| | c.425A > G (p.Lys142Arg) | | | GATCCCTACCCCTACCATGCCT′] |
| | NM_001130823.1(DNMT1): | 1786 | DNMT1 | [] |
| | c.1532A > G (p.Tyr511Cys) | | | |
| 4147 | NM_000138.4(FBN1): c.5096A > G | 2200 | FBN1 | [′AGAAGAAGTTTGTGCTACAGAAACTRCTA |
| | (p.Tyr1699Cys) | | | TGCTGACAACCAGACCTGTGAT′] |
| 4148 | NM_000138.4(FBN1): c.5087A > G | 2200 | FBN1 | [′CTAGATATGAGAAGAAGTTTGTGCTRCAG |
| | (p.Tyr1696Cys) | | | AAACTACTATGCTGACAACCAG′] |
| 4149 | NM_000138.4(FBN1): c.5099A > G | 2200 | FBN1 | [′AGAAGTTTGTGCTACAGAAACTACTRTGCT |
| | (p.Tyr1700Cys) | | | GACAACCAGACCTGTGATGGA′] |
| 4150 | NM_001244710.1(GFPT1): | 2673 | GFPT1 | [′TTACTTAAACTACCATGTTCCTCGARCGAG |
| | c.43A > G (p.Thr15Ala) | | | ACGAGAAATCCTGGAGACCCT′] |
| 4151 | NM_002292.3(LAMB2): c.440A > G | 3913 | LAMB2 | [′CTGGAGGCTGAGTTTCATTTCACACRCCTC |
| | (p.His 147Arg) | | | ATTATGACCTTCAAGGTGCCT′] |
| 4152 | NM_005211.3(CSF1R): | 1436 | CSF1R | [′GACTTAAGGGACCTGTGTGCGTGGCRGGT |
| | c.1754 − 2A > G | | | AAGACCCTCGGAGCTGGAGCCT′] |
| 4153 | NM_005247.2(FGF3): c.146A > G | 2248 | FGF3 | [′GGGGCGCCCCGGCGCCGCAAGCTCTRCTG |
| | (p.Tyr49Cys) | | | CGCCACGAAGTACCACCTCCAG′] |
| 4154 | NM_005247.2(FGF3): c.317A > G | 2248 | FGF3 | [′GCCATGAACAAGAGGGGACGACTCTRTGC |
| | (p.Tyr106Cys) | | | TTCGGTGAGTCCAGGCTGTCAC′] |
| 4155 | NM_005188.3(CBL): c.1112A > G | 867 | CBL | [′TAATCAAAGGAACAATATGAATTATRCTG |
| | (p.Tyr371Cys) | | | TGAGATGGGCTCCACATTCCAA′] |
| 4156 | NM_000313.3(PROS1): c.701A > G | 5627 | PROS1 | [′TGTGAATGCCCCGAAGGCTACAGATRTAA |
| | (p.Tyr234Cys) | | | TCTCAAATCAAAGTCTTGTGAA′] |
| 4157 | NM_000329.2(RPE65): | 6121 | RPE65 | [′TACCAGAAGTATTGTGGGAAACCTTRCAC |
| | c.1292A > G (p.Tyr431Cys) | | | ATATGCGTATGGACTTGGCTTG′] |
| 4158 | NM_001040142.1(SCN2A): | 6326 | SCN2A | [′TGAATCTTTTCATTGGTGTCATCATRGATA |
| | c.4419A > G (p.Ile 1473Met) | | | ACTTCAACCAACAGAAAAAGA′] |
| 4159 | NM_001040142.1(SCN2A): | 6326 | SCN2A | [′GTCAGTGAAGAAGCTTTCTGATGTCRTGAT |
| | c.754A > G (p.Met252Val) | | | CTTGACTGTGTTCTGTCTAAG′] |
| 4160 | NM_004612.3(TGFBR1): | 7046 | TGFBR1 | [′TTCTGCCACCTCTGTACAAAAGACARTTTT |
| | c.134A > G (p.Asn45Ser) | | | ACTTGTGTGACAGATGGGCTC′] |
| 4161 | NM_003688.3(CASK): c.2168A > G | 8573 | CASK | [′TTTGATCAATTAGATCTTGTCACATRTGAA |
| | (p.Tyr723Cys) | | | GAAGTAGTAAAACTGCCAGCA′] |
| 4162 | NM_015884.3(MBTPS2): | 51360 | MBTPS2 | [′GGTGGCAGTGTACTTTTGGCTGCCARTGTG |
| | c.1523A > G (p.Asn508Ser) | | | ACCCTGGGACTCTGGATGGTT′] |
| 4163 | m.10450A > G | 4573 | MT-TR | [′ACGAATGATTTCGACTCATTAAATTRTGAT |
| | | | | AATCATATTTACCAAATGCCC′] |
| 4164 | m.5816A > G | 4511 | MT-TC | [′GAATTTGCAATTCAATATGAAAATCRCCTC |
| | | | | GGAGCTGGTAAAAAGAGGCCT′] |
| 4165 | m.608A > G | 4558 | MT-TF | [′GTAGCTTACCTCCTCAAAGCAATACRCTGA |
| | | | | AAATGTTTAGACGGGCTCACA′] |
| 4166 | NM_172337.2(OTX2): c.674A > G | 5015 | OTX2 | [′CTGATTGAGATGGCTGGTGACTGCAYTGG |
| | (p.Asn225Ser) | | | TACCCATGGGACTGAGTGTGGC′] |
| 4167 | NM_001376.4(DYNC1H1): | 1778 | DYNC1H1 | [′CTCCTGACTCTGGATATCTTGAAACRTGGC |
| | c.917A > G (p.His306Arg) | | | AAGCGCTTCCATGCCACCGTC′] |
| 4168 | NM_001376.4(DYNC1H1): | 1778 | DYNC1H1 | [′GAAGCGGGTGGAAGATGTCCTTGGCRAGG |
| | c.2011A > G (p.Lys671Glu) | | | GCTGGGAGAATCACGTGGAGGG′] |
| 4169 | NM_001376.4(DYNC1H1): | 1778 | DYNC1H1 | [′CTAAGAATAACCAATCAGGTAATCTRCTTG |
| | c.2909A > G (p.Tyr970Cys) | | | AATCCACCAATTGAAGAGTGC′] |
| 4170 | NM_004380.2(CREBBP): | 1387 | CREBBP | [′ACTGTAGGGGTGCTCTGGGTTTGGGHAGC |
| | c.2728A > G (p.Thr910Ala) | | | ACTGGGCACTGAGCCAGGAGTC′] |
| 4171 | NM_000459.4(TEK): c.2690A > G | 7010 | TEK | [′ATGCTCTCTTCCTTCCCTCCAGGCTVCTTGT |
| | (p.Tyr897Cys) | | | ACCTGGCCATTGAGTACGCG′] |
| 4172 | NM_001354.5(AKR1C2): | 1646 | AKR1C2 | [′AGATGGCAGTGTGAAGAGAGAAGACRTAT |
| | c.235A > G (p.Ile79Val) | | | TCTACACTTCAAAGGTACTGTG′] |
| 4173 | NM_007315.3(STAT1): c.604A > G | 6772 | STAT1 | [′ACAAGAACAGCTGTTACTCAAGAAGRTGT |
| | (p.Met202Val) | | | ATTTAATGCTTGACAATAAGAG′] |
| 4174 | NM_007315.3(STAT1): c.494A > G | 6772 | STAT1 | [′GAGCATGAAATCAAGAGCCTGGAAGRTTT |
| | (p.Asp165Gly) | | | ACAAGATGAATATGACTTCAAA′] |
| 4175 | NM_007315.3(STAT1): c.862A > G | 6772 | STAT1 | [′GTTGGAGGAATTGGAACAGAAATACRCCT |
| | (p.Thr288Ala) | | | ACGAACATGACCCTATCACAAA′] |
| 4176 | NM_002052.4(GATA4): c.928A > G | 2626 | GATA4 | [′CCTCCAGGTCCCCAGGCCTCTTGCARTGCG |
| | (p.Met310Val) | | | GAAAGAGGGGATCCAAACCAG′] |
| 4177 | NM_014191.3(SCN8A): | 6334 | SCN8A | [′CATGTACATTGCCATCATCCTGGAGRACTT |
| | c.5302A > G (p.Asn1768Asp) | | | CAGTGTAGCCACAGAGGAAAG′] |
| 4178 | NM_000209.3(PDX1): c.533A > G | 3651 | PDX1 | [′TACATCTCACGGCCGCGCCGGGTGGRGCT |
| | (p.Glu178Gly) | | | GGCTGTCATGTTGAACTTGACC′] |
| 4179 | NM_000890.3(KCNJ5): c.472A > G | 3762 | KCNJ5 | [′CATTGGGTATGGCTTCCGAGTCATCRCAGA |
| | (p.Thr158Ala) | | | GAAGTGTCCAGAGGGGATTAT′] |
| 4180 | NM_000256.3(MYBPC3): | 4607 | MYBPC3 | [′ACGGTCGAGGGGGCAGAGAAGGAAGRTG |
| | c.2234A > G (p.Asp745Gly) | | | AGGGCGTCTACACGGTCACAGTG′] |
| 4181 | NM_005359.5(SMAD4): | 4089 | SMAD4 | [′TGTTGATGACCTTCGTCGCTTATGCRTACT |
| | c.1498A > G (p.Ile500Val) | | | CAGGATGAGTTTTGTGAAAGG′] |
| 4182 | NM_005359.5(SMAD4): | 4089 | SMAD4 | [′TTGATGACCTTCGTCGCTTATGCATRCTCA |
| | c.1500A > G (p.Ile500Met) | | | GGATGAGTTTTGTGAAAGGCT′] |
| 4183 | NM_001184.3(ATR): c.6431A > G | 545 | ATR | [′TATCAATTTTTGACTGCTTTTTCACRATTGA |
| | (p.Gln2144Arg) | | | TCTCTCGAATTTGTCATTCT′] |
| 4184 | NM_153427.2(PITX2): c.262A > G | 5308 | PITX2 | [′CCCCAACCGCCCCCAGGTTTGGTTCRAGAA |
| | (p.Lys88Glu) | | | TCGTCGGGCCAAATGGAGAAA′] |
| 4185 | NM_005257.5(GATA6): | 2627 | GATA6 | [′CTGTGCCAACTGTCACACCACAACTRCCAC |
| | c.1354A > G (p.Thr452Ala) | | | CTTATGGCGCAGAAACGCCGA′] |
| 4186 | NM_005257.5(GATA6): | 2627 | GATA6 | [′AAACGCCGAGGGTGAACCCGTGTGCVATG |
| | c.1396A > G (p.Asn466Asp) | | | CTTGTGGACTCTACATGAAACT′] |
| 4187 | NM_000414.3(HSD17B4): | 3295 | HSD17B4 | [′CTTGTGGAAGCCCTGAAGCCAGAGTRTGT |
| | c.650A > G (p.Tyr217Cys) | | | GGCACCTCTTGTCCTTTGGCTT′] |
| 4188 | NM_004153.3(ORC1): c.380A > G | 4998 | ORC1 | [′GCCTGTGACAGCAACATTAATGCGGRGAC |
| | (p.Glu127Gly) | | | CATCATTGGCCTTGTTCGGGTA′] |
| 4189 | NM_002552.4(ORC4): c.521A > G | 5000 | ORC4 | [′CATCATAAAAACCAAACACTTCTCTRTAAT |
| | (p.Tyr174Cys) | | | CTTTTTGACATTTCTCAGTCT′] |
| 4190 | NM_004656.3(BAP1): | 8314 | BAP1 | [′GCTCCACTAGGTTGGCCAGCATGCCYGCG |
| | c.2057 − 2A > G | | | AAGAGGTAGAGACCCTTGAGCA′] |
| 4191 | NM_004656.3(BAP1): | 8314 | BAP1 | [′TCAGGGAGGTGGCGTGGCTCGGGCCYGGG |
| | c.438 − 2A > G | | | GAAAAACAGAGTCAGGGCCCAA′] |
| | NM_003590.4(CUL3): c.1238A > G | 8452 | CUL3 | [] |
| | (p.Asp413Gly) | | | |
| 4192 | NM_004813.2(PEX16): c.992A > G | 9409 | PEX16 | [′TACTTGCCCACCTGGCAGAAAATCTRCTTC |
| | (p.Tyr331Cys) | | | TACAGTTGGGGCTGACAGACC′] |
| 4193 | NM_004544.3(NDUFA10): c.1A > G | 4705 | NDUFA10 | [′TTGATCCTGAGCTGACCGGGTAGCCRTGGC |
| | (p.Met1Val) | | | CTTGCGGCTCCTGAAGCTGGC′] |
| 4194 | NM_004544.3(NDUFA10): | 4705 | NDUFA10 | [′TGGTTGTACAGCAGTCGCCTGCTGCRGTAC |
| | c.425A > G (p.Gln142Arg) | | | TCAGATGCCTTGGAGCACTTG′] |
| 4195 | NM_006796.2(AFG3L2): | 10939 | AFG3L2 | [′TATTTTTCAGATTGTTCAGTTTGGCDTGAA |
| | c.1996A > G (p.Met666Val) | | | TGAAAAGGTTGGGCAAATCTC′] |
| 4196 | NM_006796.2(AFG3L2): | 10939 | AFG3L2 | [′TATTTACCAAAAGAACAATACCTCTRTACC |
| | c.1847A > G (p.Tyr616Cys) | | | AAAGAGCAGCTCTTGGATAGG′] |
| 4197 | NM_001199397.1(NEK1): | 4750 | NEK1 | [′CAAAATTCTTTGTATCTTTTCATCTRGCTA |
| | c.869 − 2A > G | | | AAAGACCAGCTTCAGGACAAA′] |
| 4198 | NM_006587.3(CORIN): c.949A > G | 10699 | CORIN | [′GAATCTGTTTCACTGTCACACAGGCRAGTG |
| | (p.Lys317Glu) | | | CCTTAATTACAGCCTTGTGTG′] |
| 4199 | NM_006587.3(CORIN): | 10699 | CORIN | [′CATGAATTTGCCCTACAACAGTACARGTTA |
| | c.1414A > G (p.Ser472Gly) | | | TCCAAATTATTTTGGCCACAG′] |
| 4200 | NM_015560.2(OPA1): c.1294A > G | 4976 | OPA1 | [′AGCTTACATGCAGAATCCTAATGCCRTCAT |
| | (p.Ile432Val) | | | ACTGTGTATTCAAGGTAAATC′] |
| 4201 | NM_021625.4(TRPV4): | 59341 | TRPV4 | [′CGTGGGCTGAAGCTGACGGGGACCTRTAG |
| | c.1805A > G (p.Tyr602Cys) | | | CATCATGATCCAGAAGGTACGG′] |
| 4202 | NM_021625.4(TRPV4): c.590A > G | 59341 | TRPV4 | [′TCTACGGGGAAGACCTGCCTGCCCARGGC |
| | (p.Lys197Arg) | | | CTTGCTGAACCTGAGCAATGGC′] |
| 4203 | NM_021625.4(TRPV4): c.826A > G | 59341 | TRPV4 | [′GGCCCGTGGGCGCTTCTTCCAGCCCRAGG |
| | (p.Lys276Glu) | | | ATGAGGGGGGCTACTTCTACTT′] |
| 4204 | NM_024022.2(TMPRSS3): | 64699 | TMPRSS3 | [′GTCTCGGATTGCAAAGACGGGGAGGREGA |
| | c.308A > G (p.Asp103Gly) | | | GTACCGCTGTGGTAAGGTCATG′] |
| 4205 | NM_016599.4(MYOZ2): c.738A > G | 51778 | MYOZ2 | [′GATGGATATCTGAGAATATTCCTATRGTGA |
| | (p.Ile246Met) | | | TAACAACCGAACCTACAGATG′] |
| | NM_017415.2(KLHL3): | 26249 | KLHL3 | [] |
| | c.1670A > G (p.Tyr557Cys) | | | |
| 4206 | NM_019109.4(ALG1): c.1129A > G | 56052 | ALG1 | [′GTCCTCCAGTGGCCTGGACCTGCCCRTGAA |
| | (p.Met377Val) | | | GGTGGTGGACATGTTCGGGTG′] |
| 4207 | NM_031885.3(BBS2): | 583 | BBS2 | [′GCTAATGGTTTGGGGTTTTATTTTCRGGTT |
| | c.472 − 2A > G | | | ACTGGAGACAATGTTAATTCC′] |
| 4208 | NM_006886.3(ATP5E): c.35A > G | −1 | — | [′TACCTTCTCTCATTTTCTCCCAGCTRCATCC |
| | (p.Tyr12Cys) | | | GATACTCCCAGATCTGTGCA′] |
| 4209 | NM_022445.3(TPK1): c.656A > G | 27010 | TPK1 | [′CACAACACCAGACCCGTCGTAGGTAYTGG |
| | (p.Asn219Ser) | | | AAGTACTGACCAATGTTCCAAA′] |
| 4210 | NM_000017.3(ACADS): | 35 | ACADS | [′CTCTGACTGTACCCCCATGTTTAGGRGGCA |
| | c.1031A > G (p.Glu344Gly) | | | GCCATGGCCAAGCTGGCCGCC′] |
| 4211 | NM_016013.3(NDUFAF1): | 51103 | NDUFAF1 | [′GGGGGACCCTACTGGCAGGAGGTCARGGT |
| | c.758A > G (p.Lys253Arg) | | | AACAGCATAAATCTTCATTGTT′] |
| 4212 | NM_032580.3(HES7): c.172A > G | 84667 | HES7 | [′GAACCCGAAGCTGGAGAAAGCGGAGRTAT |
| | (p.Ile58Val) | | | TGGAGTTCGCCGTGGGCTACTT′] |
| 4213 | NM_024887.3(DHDDS): c.124A > G | 79947 | DHDDS | [′GGACGGGAACCGTCGCTATGCCAAGRAGT |
| | (p.Lys42Glu) | | | GCCAGGTGGAGCGGCAGGAAGG′] |
| 4214 | NM_024700.3(SNIP1): | 79753 | SNIP1 | [′CTCAAATTTGGATTCAGTAGCAGAGRATA |
| | c.1097A > G (p.Glu366Gly) | | | CGTCTTGCTCCATGAGTCGTCG′] |
| 4215 | NM_206933.2(USH2A): | 7399 | USH2A | [′AATTGAACACCTCTCCTTTCCCAAGRTAAG |
| | 2c.7595 − 144A > G | | | AGATCATCTTTAAGAAAAGGC′] |
| 4216 | NM_016952.4(CDON): c.2368A > G | 50937 | CDON | [′GTTTTTGTTTTCCCTCAAAGGTTCARCATA |
| | (p.Thr790Ala) | | | CAAATTTAGGGTCATTGCCAT′] |
| 4217 | NM_020435.3(GJC2): | 57165 | GJC2 | [′AGACCCTGAGGCCGAGGGGGGAACARTGG |
| | c.−167A > G | | | GGCCCTTGAGGGCCCCTCCTCC′] |
| 4218 | NM_024549.5(TCTN1): | 79600 | TCTN1 | [′TGGATCCTACCCCTCTTTTTTCTGCRGTTGC |
| | c.221 − 2A > G | | | TGTTCTCTGTGTCTGTGACT′] |
| 4219 | NM_031427.3(DNAL1): c.449A > G | 83544 | DNAL1 | [′CTCGAAGACCTGGTGTTTGTAGGCARTCCC |
| | (p.Asn150Ser) | | | TTGGAAGAGAAACATTCTGCT′] |
| 4220 | NM_145046.4(CALR3): c.245A > G | 125972 | CALR3 | [′AGTTTTCCCTTTATTGCTGAACGGTYTGAA |
| | (p.Lys82Arg) | | | GCGTGCAGAGATGGCATAGAA′] |
| 4221 | NM_020320.3(RARS2): | 57038 | RARS2 | [′AATGGACAAGTATAATTTTGATACARTGAT |
| | c.1024A > G p.Met342Val) | | | ATATGTGGTAAGTAATCAGAA′] |
| 4222 | NM_020320.3(RARS2): c.35A > G | 57038 | RARS2 | [′CTCTGCGCGCTCCGGGATCCATACCYGGC |
| | (p.Gln12Arg) | | | AAGCAATAGCGCGGCGAAAGCC′] |
| 4223 | NM_001017361.2(KHDC3L): | 154288 | KHDC3L | [′TGTCTCCTGCAGGACCGGCCGCAGCRTGG |
| | c.1A> G (p.Met1Val) | | | ACGCTCCCAGGCGGTTTCCGAC′] |
| 4224 | NM_024753.4(TTC21B): | 79809 | TTC21B | [′TAATCGTGCCAGTTCCAACATAATCYGTAG |
| | c.2758 − 2A > G | | | AGCAAAGGGCTAGATTCATCA′] |
| 4225 | NM_001145901.1(SARS2): | 54938 | SARS2 | [′TTCGTCCTTGACCGCAGGGTCCTGGRTATG |
| | c.1175A > G (p.Asp392Gly) | | | CCCACCCAAGAACTGGGCCTC′] |
| 4226 | NM_000552.3(VWF): c.3437A > G | 7450 | VWF | [′AACGGGTATGAGTGTGAGTGGCGCTRTAA |
| | (p.Tyr1146Cys) | | | CAGCTGTGCACCTGCCTGTCAA′] |
| 4227 | NM_014985.3(CEP152): | 22995 | CEP152 | [′ACCTTACCTATCCACAGCTTCTTGTYTGTC |
| | c.2000A > G (p.Lys667Arg) | | | ATGGTCAAAATCTTGTACCAT′] |
| 4228 | NM_024809.4(TCTN2): | 79867 | TCTN2 | [′GCTGAGAAATGTCTTACTCTCTTGCRGGGA |
| | c.1506 − 2A > G | | | GAATGCTGTTGAAAGACTTGA′] |
| 4229 | NM_198994.2(TGM6): c.980A > G | 343641 | TGM6 | [′CGGACCCTGGAGGACCTGACAGAAGRCAG |
| | (p.Asp327Gly) | | | CATGTGGTGGGTCCTGCCCCCA′] |
| 4230 | NM_052873.2(IFT43): c.1A > G | 112752 | IFT43 | [′GAAGTGACGTCAGGCGGCCGCGGAGRTGG |
| | (p.Met1Val) | | | AGGATTTGCTCGACTTGGACGA′] |
| 4231 | NM_201269.2(ZNF644): | 84146 | ZNF644 | [′AACATTTGGATCAACCTCACAATCARGTA |
| | c.2014A > G (p.Ser672Gly) | | | GTTTTTCAAAAATTCATAAGCG′] |
| 4232 | NM_201269.2(ZNF644): | 84146 | ZNF644 | [′GTAGTAAAAGGACACATCTTACATAYGTA |
| | c.1759A > G (p.Ile587Val) | | | GGTAGCTGATTTTTTGGATGAT′] |
| 4233 | NM_018699.3(PRDM5): c.320A > G | 11107 | PRDM5 | [′TTTCAGGAAGGAGAAAACATTTTCTRTTTG |
| | (p.Tyr107Cys) | | | GCAGTTGAAGATATAGAAACA′] |
| 4234 | NM_001198799.2(ASCC1): | 51008 | ASCC1 | [′CTATAGTTCTCTGGGGGACTTACCAYTGGG |
| | c.953A > G (p.Asn318Ser) | | | GTCTTTCCTGAATAGTGTATT′] |
| 4235 | NM_007055.3(POLR3A): | 11128 | POLR3A | [′ACCAACTGAGTTTTTCTTCCACACARTGGC |
| | c.2554A > G (p.Met852Val) | | | CGGCCGGGAAGGTCTAGTCGA′] |
| 4236 | NM_001256047.1(C19orf12): | 83636 | C19orf12 | [′TACTGGATCTCGGCCCGCAGCTCCTYGGTG |
| | c.391A > G (p.Lys131Glu) | | | ACGTAGTTCACCAGCATGGCC′] |
| 4237 | NM_016464.4(TMEM138): | 51524 | TMEM138 | [′TACTTTGCCCTCAGCATCTCCCTTCRTGTCT |
| | c.287A > G (p.His96Arg) | | | GGGTCATGGTAAGAGTGGCA′] |
| 4238 | NM_016464.4(TMEM138): | 51524 | TMEM138 | [′TTCCTCCCCACAGCAGCAGTGTTGTRCTGC |
| | c.389A > G (p.Tyr130Cys) | | | TACTTCTATAAACGGACAGCC′] |
| 4239 | NM_177965.3(C8orf37): | 157657 | C8orf37 | [′AAAGGAACACGGGCATATGCCTGCCRGTG |
| | c.545A > G (p.Gln182Arg) | | | TAGCTGGAGAACTATTGAAGAA′] |
| 4240 | NM_001004334.3(GPR179): | 440435 | GPR179 | [′CCCAAGTGGCCGCAGGCAGATGGATRTGT |
| | c.659A > G (p.Tyr220Cys) | | | GGGGGACACGCAGCAGGTGAGG′] |
| 4241 | NM_004963.3(GUCY2C): | 2984 | GUCY2C | [′AGCAGTGAGACATACTTTCTTGGTGYCCAC |
| | c.1160A > G (p.Asp387Gly) | | | AGAGGTATACAGAAGCACCAT′] |
| 4242 | NM_024110.4(CARD14): | 79092 | CARD14 | [′AGCCTGCAGGAGGAGCTGAACCAGGRAAA |
| | c.425A > G (p.Glu142Gly) | | | GGGGCAGAAGGAGGTGCTGCTG′] |
| 4243 | NM_018105.2(THAP1): c.70A > G | 55145 | THAP1 | [′CAAGGACAAGCCCGTTTCTTTCCACRAGTG |
| | (p.Lys24Glu) | | | AGGACCCTGCGCGCCTCGCGG′] |
| 4244 | NM_000933.3(PLCB4): | 5332 | PLCB4 | [′AACAAACGGCAAATGAGTCGCATTTRCCC |
| | c.1868A > G (p.Tyr623Cys) | | | CAAGGGAGGCCGAGTCGATTCC′] |
| 4245 | NM_001172646.1(PLCB4): | 5332 | PLCB4 | [′CACTACTTCATCAGTTCTTCCCATAVCACT |
| | c.986A > G (p.Asn329Ser) | | | TATCTCACTGGCAGACAGTTC′] |
| 4246 | NM_005850.4(SF3B4): c.1A > G | 10262 | SF3B4 | [′GGGAGACGGCGGGATCTCTTTCGCCRTGG |
| | (p.Met1Val) | | | CTGCCGGGCCGATCTCCGAGCG′] |
| 4247 | NM_014714.3(IFT140): | 9742 | IFT140 | [′TGGGACATAGAACGAGGAGAGAATTRTAT |
| | c.932A > G (p.Tyr311Cys) | | | ACTGAGTCCAGATGAGAAGTTT′] |
| 4248 | NM_000258.2(MYL3): c.517A > G | 4634 | MYL3 | [′GACAGAAGACGAAGTGGAGAAGTTGRTGG |
| | (p.Met173Val) | | | CTGGGCAAGAGGACTCCAATGG′] |
| 4249 | NM_032578.3(MYPN): c.59A > G | 84665 | MYPN | [′ATATCTCAGCTTCTAAGAGAGAGCTRTTTA |
| | (p.Tyr20Cys) | | | GCTGAAACCAGACATCGGGGA′] |
| 4250 | NM_001018005.1(TPM1): | 7168 | TPM1 | [′TGAGTTTGCGGAGAGGTCAGTAACTRAAT |
| | c.742A > G (p.Lys248Glu) | | | TGGAGAAAAGCATTGATGACTT′] |
| 4251 | NM_005006.6(NDUFS1): | 4719 | NDUFS1 | [′AGCTGCTTACACAGAGAAGTCTGCTRCAT |
| | Gc.1783A > (p.Thr595Ala) | | | ATGTCAACACTGAGGGTAGAGC′] |
| 4252 | NM_033360.3(KRAS): c.439A > G | 3845 | KRAS | [′AATTCCTTTTATTGAAACATCAGCARAGAC |
| | (p.Lys147Glu) | | | AAGACAGGTAAGTAACACTGA′] |
| 4253 | NM_014236.3(GNPAT): | 8443 | GNPAT | [′GAAGATGAACTCATCTGCAAAAACAHCAC |
| | c.1556A > G (p.Asp519Gly) | | | GTAGGAAGCGAAAGCAACTGTA′] |
| 4254 | NM_017565.3(FAM20A): | −1 | — | [′GGCACCCGTCGGAAGTCCAGAATCCYGCA |
| | c.813 − 2A > G | | | AGAGAGGAAGCTCTGTTCCATC′] |
| 4255 | NM_017565.3(FAM20A): | 54757 | FAM20A | [′AGGCTTTCTCATCTTGACTGTAATCYGCAA |
| | c.590 − 2A > G | | | AGGAGGAGAAGGGCAATGAGA′] |
| 4256 | NM_017636.3(TRPM4): | 54795 | TRPM4 | [′CTGCTTCACATCTTCACGGTCAACARACAG |
| | c.2741A > G (p.Lys914Arg) | | | CTGGGGCCCAAGATCGTCATC′] |
| 4257 | NM_000076.2(CDKNIC): | 1028 | CDKNIC | [′GCTGTCGCCCGCAGATTTCTTCGCCRAGCG |
| | Gc.832A > (p.Lys278Glu) | | | CAAGAGATCAGCGCCTGAGAA′] |
| 4258 | NM_022912.2(REEP1): | 65055 | REEP1 | [′AGTGTGCCTCTGTTTTTCCTTTGACRGGAA |
| | c.304 − 2A > G | | | ATCGATGATTGTCTGGTCCAA′] |
| 4259 | NM_000053.3(ATP7B): | 540 | ATP7B | [′TGTGACATTCTTCGACACGCCCCCCRTGCT |
| | c.2305A > G (p.Met769Val) | | | CTTTGTGTTCATTGCCCTGGG′] |
| 4260 | NM_000053.3(ATP7B): | 540 | ATP7B | [′GTCCACACCCATGCTCTGCAGCGTGYGCA |
| | c.3620A > G (p.His1207Arg) | | | CAGCCAGGGCAGCCTCCTGCTT′] |
| 4261 | NM_000492.3(CFTR): | 1080 | CFTR | [′ACATTTTGTGTTTATGTTATTTGCARTGTTT |
| | c.3140 − 26A > G | | | TCTATGGAAATATTTCACAG′] |
| 4262 | NM_000570.4(FCGR3B): | 2215 | FCGR3B | [′GCTCGAGGCCTGGCTTGAGATGAGGYTCT |
| | Gc.194A > (p.Asn65Ser) | | | CATTGTGAAACCACTGTGTGGA′] |
| 4263 | NM_198253.2(TERT): c.2705A > G | 7015 | TERT | [′TATGGCTGCGTGGTGAACTTGCGGARGAC |
| | (p.Lys902Arg) | | | AGTGGTGAACTTCCCTGTAGAA′] |
| 4264 | NM_005022.3(PFN1): c.350A > G | 5216 | PFN1 | [′GTTGATCAAACCACCGTGGACACCTYCTTT |
| | (p.Glu117Gly) | | | GCCCATCAGCAGGACTAGCGC′] |
| 4265 | NM_005349.3(RBPJ): c.188A > G | 3516 | RBPJ | [′GTTGCACAGAAGTCATATGGAAATGRAAA |
| | (p.Glu63Gly) | | | AAGGTAAGATTATTTTTCTGGT′] |
| 4266 | NM_005349.3(RBPJ): c.505A > G | 3516 | RBPJ | [′GATAAAAGTCATCTCCAAACCTTCCRAAA |
| | (p.Lys169Glu) | | | AGAAGCAGTCATTGAAAAATGC′] |
| 4267 | NM_022787.3(NMNAT1): | 64802 | NMNAT1 | [′GGTCATCCTGGCCCCTTTGCAGAGARACAC |
| | c.817A > G (p.Asn273Asp) | | | TGCAGAAGCTAAGACATAGGA′] |
| 4268 | NM_020921.3(NIN): c.3665A > G | 51199 | NIN | [′CACAGAAACATCAAAAAGTAGGTCCYGTT |
| | (p.Gln1222Arg) | | | TCTTTTCAGAAGCTCGATCACA′] |
| 4269 | NM_020921.3(NIN): c.5126A > G | 51199 | NIN | [′AAAATCTCTAGTGTTCTAAGCTACARCGAA |
| | (p.Asn1709Ser) | | | AAACTGCTGAAAGAAAAGGAA′] |
| 4270 | NM_005340.6(HINT1): c.152A > G | 3094 | HINT1 | [′GACATTTCCCCTCAAGCACCAACACRTTTT |
| | (p.His51Arg) | | | CTGGTGATACCCAAGAAACAT′] |
| 4271 | NM_005050.3(ABCD4): c.956A > G | 5826 | ABCD4 | [′GAGCTGGGTGAAGCAGCTGATGAGGYAGA |
| | (p.Tyr319Cys) | | | TGCACACAAAGGCATTCTGGAC′] |
| 4272 | NM_000155.3(GALT): c.950A > G | 2592 | GALT | [′GCTGGGGCCAACTGGAACCATTGGCRGCT |
| | (p.Gln317Arg) | | | GCACGCTCATTACTACCCTCCG′] |
| 4273 | NM_007294.3(BRCA1): | 672 | BRCA1 | [′ACTTGCTGAGTGTGTTTCTCAAACARTTTA |
| | c.213 − 12A > G | | | ATTTCAGGAGCCTACAAGAAA′] |
| 4274 | NM_007294.3(BRCA1): | 672 | BRCA1 | [′GCAAAGCATGGATTCAAACTTAGGTRTTG |
| | c.4096 + 3A> G | | | GAACCAGGTTTTTGTGTTTGCC′] |
| 4275 | NM_007294.3(BRCA1): | 672 | BRCA1 | [′TGATGGGTTGTGTTTGGTTTCTTTCVGCAT |
| | c.5194 − 2A > G | | | GATTTTGAAGTCAGAGGAGAT′] |
| 4276 | NM_007294.3(BRCA1): | 672 | BRCA1 | [′GTGCAGCCAGATGCCTGGACAGAGGRCAA |
| | c.5453A > G (p.Asp1818Gly) | | | TGGCTTCCATGGTAAGGTGCCT′] |
| 4277 | NM_000059.3(BRCA2): | 675 | BRCA2 | [′TATGCTATACATGATGAAACATCTTRTAAA |
| | c.1799A > G (p.Tyr600Cys) | | | GGAAAAAAAATACCGAAAGAC′] |
| 4278 | NM_000059.3(BRCA2): | 675 | BRCA2 | [′ACAATTTTCCCCTTTTTTTACCCCCRGTGGT |
| | c.476 − 2A > G | | | ATGTGGGAGTTTGTTTCATA′] |
| 4279 | NM_000059.3(BRCA2): | 675 | BRCA2 | [′AAAGTGGCCATTATTGAACTTACAGNTGG |
| | c.8168A > G (p.Asp2723Gly) | | | GTGGTATGCTGTTAAGGCCCAG′] |
| 4280 | NM_000059.3(BRCA2): | 675 | BRCA2 | [′GAATTAATAATCCTTTTGTTTTCTTDGAAA |
| | c.8633 − 2A > G | | | ACACAACAAAACCATATTTAC′] |
| 4281 | NM_000060.3(BTD): c.629A > G | 686 | BTD | [′AATAATGGAACCCTTGTTGACCGCTRCCGT |
| | (p.Tyr210Cys) | | | AAACACAACCTCTACTTTGAG′] |
| 4282 | NM_000060.3(BTD): c.968A > G | 686 | BTD | [′ACCCCTCTGGAGTCCTTTTGGTACCRTGAC |
| | (p.His323Arg) | | | ATGGAAAATCCCAAAAGTCAC′] |
| 4283 | NM_000155.3(GALT): c.857A > G | 2592 | GALT | [′ATCATGAAGAAGCTCTTGACCAAGTRTGA |
| | (p.Tyr286Cys) | | | CAACCTCTTTGAGACGTCCTTT′] |
| 4284 | NM_000060.3(BTD): c.128A > G | 686 | BTD | [′TGTTACGTGGTTGCCCTGGGAGCCCRCACC |
| | (p.His43Arg) | | | GGGGAGGAGAGCGTGGCTGAC′] |
| 4285 | NM_002437.4(MPV17): c.262A > G | 4358 | MPV17 | [′CACCACCAAAGTGGATGCACTGAAGRAGA |
| | (p.Lys88Glu) | | | TGTTGTTGGATCAGGTGAGCAG′] |
| 4286 | NM_002769.4(PRSS1): c.65A > G | −1 | — | [′GTTGCTGCCCCCTTTGATGATGATGRCAAG |
| | (p.Asp22Gly) | | | ATCGTTGGGGGCTACAACTGT′] |
| 4287 | NM_005211.3(CSF1R): | 1436 | CSF1R | [′GACTAACCCTGCAGTGCTTTCCCTCRGTGC |
| | 2c.2320 − A > G | | | ATCCACCGGGACGTGGCAGCG′] |
| 4288 | NM_024312.4(GNPTAB): | 79158 | GNPTAB | [′GAAAACGTTTCTTTTTCTTTGTTCTRGGTG |
| | c.118 − 2A > G | | | GTTCTGGAATGGAGCCGAGAT′] |
| 4289 | NM_000495.4(COL4A5): | 1287 | COL4A5 | [′AAATATTATATATCACATATTTTCARCAGG |
| | c.3107 − 4A > G | | | GCCTCAGGGTGTGGAAGGGCC′] |
| 4290 | NM_000282.3(PCCA): c.862A > G | 5095 | PCCA | [′TGGGAATGCTTTATGGCTTAATGAANGAG |
| | (p.Arg288Gly) | | | AGTGCTCAATTCAGAGAAGAAA′] |
| 4291 | NM_000288.3(PEX7): c.854A > G | 5191 | PEX7 | [′TCTCTTCTTGAAACAGTGGAGCATCRTACA |
| | (p.His285Arg) | | | GAGTTTACTTGTGGTTTAGAC′] |
| 4292 | NM_000532.4(PCCB): c.1606A > G | 5096 | PCCB | [′ACAACGTCCTTGGAGAAAACATGCARATA |
| | (p.Asn536Asp) | | | TTCCATTGTAAACAAATCAAAG′] |
| 4293 | NM_000553.4(WRN): | 7486 | WRN | [′TCAAAGGAAAAATACATTTAAGATTVTAA |
| | c.2089 − 3024A > G | | | GTCTGGTTATAAGCTTAAAAAG′] |
| 4294 | NM_001039958.1(MESP2): | 145873 | MESP2 | [′GCGGCAGAGCGCCAGCGAGCGGGAGRAA |
| | c.271A > G (p.Lys91Glu) | | | CTGCGCATGCGCACGCTGGCCCG′] |
| 4295 | NM_001099274.1(TINF2): | 26277 | TINF2 | [′TAGGGGAGGCCATAAGGAGCGCCCCRCAG |
| | c.850A > G (p.Thr284Ala) | | | TCATGCTGTTTCCCTTTAGGAA′] |
| 4296 | NM_001099274.1(TINF2): | 26277 | TINF2 | [′CCCCACAGTCATGCTGTTTCCCTTTRGGAA |
| | c.871A > G (p.Arg291Gly) | | | TCTCGGCTCACCAACCCAGGT′] |
| 4297 | NM_001363.4(DKC1): c.115A > G | 1736 | DKC1 | [′ACAACACGCTGAAGAATTTCTTATCRAACC |
| | (p.Lys39Glu) | | | TGAATCCAAAGTTGCTAAGTT′] |
| 4298 | NM_001363.4(DKC1): c.127A > G | 1736 | DKC1 | [′AGAATTTCTTATCAAACCTGAATCCRAAGT |
| | (p.Lys43Glu) | | | TGCTAAGTTGGACACGTCTCA′] |
| 4299 | NM_001363.4(DKC1): c.196A > G | 1736 | DKC1 | [′GAATTTTGATAAGCTGAATGTAAGGRCAA |
| | (p.Thr66Ala) | | | CACACTATACACCTCTTGCATG′] |
| 4300 | NM_001363.4(DKC1): c.941A > G | 1736 | DKC1 | [′GTAAATGCCATCTGCTATGGGGCCARGATT |
| | (p.Lys314Arg) | | | ATGCTTCCAGGTGTTCTTCGA′] |
| 4301 | NM_004614.4(TK2): c.173A > G | 7084 | TK2 | [′GCATGTCGTCTTCCCACTTGCAATAYTGCC |
| | (p.Asn58Ser) | | | CTCGACACAGATCTGGCAAAA′] |
| 4302 | NM_004614.4(TK2): c.278A > G | 7084 | TK2 | [′AACCAACAACCCACTCACCAGAGGAYTGT |
| | (p.Asn93Ser) | | | GGCCACGGACATTTCTCCACTT′] |
| 4303 | NM_004614.4(TK2): c.562A > G | 7084 | TK2 | [′AGTTTACCTTCGGACCAATCCTGAGRCTTG |
| | (p.Thr188Ala) | | | TTACCAGAGGTTAAAGAAGAG′] |
| 4304 | NM_024312.4(GNPTAB): | 79158 | GNPTAB | [′TTTATTTTCTCTTTGGTTTTATCGTRGGTTT |
| | c.1285 − 2A > G | | | ATTTGACATGGCCTGTGCCA′] |
| 4305 | NM_024312.4(GNPTAB): | 79158 | GNPTAB | [′TTCACATCGCGGAAAGTCCCTGCTCRCATG |
| | c.2867A > G (p.His956Arg) | | | CCTCACATGATTGACCGGATT′] |
| 4306 | NM_024312.4(GNPTAB): | 79158 | GNPTAB | [′AGGAAGTTTGTTTGCCTGAATGACARCATT |
| | c.3458A > G (p.Asn1153Ser) | | | GACCACAATCATAAAGATGCT′] |
| 4307 | NM_024312.4(GNPTAB): | 79158 | GNPTAB | [′AGCAAAAATACTGGGAGGCAACTAARAGA |
| | c.2783A > G (p.Lys928Arg) | | | TACATTTGCAGATTCCCTCAGA′] |
| 4308 | NM_198253.2(TERT): c.2537A > G | 7015 | TERT | [′TCCACGCTGCTCTGCAGCCTGTGCTRCGGC |
| | (p.Tyr846Cys) | | | GACATGGAGAACAAGCTGTTT′] |
| 4309 | NM_198578.3(LRRK2): | 120892 | LRRK2 | [′TAGAGAAACTGGAGCAGCTCATTTTRGAA |
| | c.3342A > G (p.Leu1114=) | | | GGGTAAGAAAGAGCTCATTAAA′] |
| 4310 | NM_198578.3(LRRK2): | 120892 | LRRK2 | [′TTTGGCTGACCTGCCTAGAAATATTRTGTT |
| | c.5605A > G (p.Met1869Val) | | | GAATAATGATGAGTTGGAATT′] |
| 4311 | NM_207352.3(CYP4V2): | 285440 | CYP4V2 | [′TTCAAAGCAAATTGACAAATCCTCTRTGTA |
| | c.367A > G (p.Met123Val) | | | CAAGTTTTTAGAACCATGGCT′] |
| 4312 | NM_207352.3(CYP4V2): | 285440 | CYP4V2 | [′CTTATGTTTAAAGAAGGATGGGAACRCAA |
| | c.761A > G (p.His254Arg) | | | AAAGAGCCTTCAGATCCTACAT′] |
| 4313 | NR_001566.1(TERC): n.37A > G | 7012 | TERC | [′GGTGGGCCTGGGAGGGGTGGTGGCCDTTT |
| | | | | TTTGTCTAACCCTAACTGAGAA′] |
| 4314 | NR_001566.1(TERC): n.48A > G | 7012 | TERC | [′GAGGGGTGGTGGCCATTTTTTGTCTRACCC |
| | | | | TAACTGAGAAGGGCGTAGGCG′] |
| 4315 | NM_007171.3(POMT1): c.430A > G | 10585 | POMT1 | [′CATGTGTTTCCTCTTTGAAACAGAGRATGC |
| | (p.Asn144Asp) | | | TCTCATCACTCAGTCAAGGCT′] |
| 4316 | NM_003863.3(DPM2): c.68A > G | 8818 | DPM2 | [′GCCGTTAGCCTGATCATCTTCACCTRCTAC |
| | (p.Tyr23Cys) | | | ACCGCCTGGGTGATTCTCTTG′] |
| 4317 | NM_000030.2(AGXT): c.1020A > G | 189 | AGXT | [′GGAGAGACATCGTCAGCTACGTCATRGAC |
| | (p.Ile340Met) | | | CACTTCGACATTGAGATCATGG′] |
| 4318 | NM_031418.2(ANO3): c.2053A > G | 63982 | ANO3 | [′TTATGTTCTATTTCAGTGTCATCCTRGTGG |
| | (p.Ser685Gly) | | | CTGTTTGATAGACCTCTGCCT′] |
| 4319 | NM_183075.2(CYP2U1): | 113612 | CYP2U1 | [′CCTTTTTACATAGAAAAGGTTCATGRAGAA |
| | c.1139A > G (p.Glu380Gly) | | | ATTGAAAGAGTCATTGGCGCC′] |
| 4320 | NM_003094.3(SNRPE): c.1A > G | 6635 | SNRPE | [′GGTGTGCTCTTTGTGAAATTCCACCRTGGC |
| | (p.Met1Val) | | | GTACCGTGGCCAGGGTCAGAA′] |
| 4321 | NM_000344.3(SMN1): c.389A > G | 6606 | SMN1 | [′ACCTGTGTTGTGGTTTACACTGGATRTGGA |
| | (p.Tyr130Cys) | | | AATAGAGAGGAGCAAAATCTG′] |
| 4322 | NM_012082.3(ZFPM2): | −1 | — | [′CATGCAGAGAACCATGCGCACACGCRAGC |
| | c.2209A > G (p.Lys737Glu) | | | GCAGAAAGATGTATGAGATGTG′] |
| 4323 | NM_006364.2(SEC23A): | 10484 | SEC23A | [′TGTTCAGTGTCAATGTATCTTGGCAYTGGA |
| | Gc.2104A > G (p.Met702Val) | | | AATCTGGAGTGAAGAATTTCC′] |
| 4324 | NM_001139.2(ALOX12B): | 242 | ALOX12B | [′ACCCTCCACGGCTGCGTCACTCGGGYAAT |
| | c.1562A > G (p.Tyr521Cys) | | | AATAGGTGATGATCTCCGTCAC′] |
| 4325 | NM_001004434.2(SLC30A2): | 7780 | SLC30A2 | [′GTGACTGTCAGGACCCTTCTGAGCAYGGC |
| | c.161A > G (p.His54Arg) | | | AGTGATGGTTGCTCTGGGCAGC′] |
| 4326 | NM_001168272.1(ITPR1): | 3708 | ITPR1 | [′CACTATCACTGCCCTGCTCCACAATRATCG |
| | c.1759A > G (p.Asn587Asp) | | | GAAACTCCTGGAAAAACACAT′] |
| 4327 | m.15923A > G | −1 | — | [′ATAAACTAATACACCAGTCTTGTAARCCG |
| | | | | GAGATGAAAACCTTTTTCCAAG′] |
| 4328 | NM_007332.2(TRPA1) | −1 | — | [′TTTTTTTACTTTTCTAGATTTGAAARTTGTG |
| | c.2564A > G (p.Asn855Ser) | | | GAATTTTTATTGTTATGTTG′] |
| 4329 | NM_014254.2(TMEM5): | 10329 | TMEM5 | [′CCGGTCGGAGTAAACACAGAATGCTRTCG |
| | c.1016A > G (p.Tyr339Cys) | | | AATCTATGAGGCTTGCTCCTAT′] |
| 4330 | NM_144577.3(CCDC114): | 93233 | CCDC114 | [′ACTGATTTTTTTCTCCTCCCCCCACRGGAG |
| | c.487 − 2A > G | | | ATCCACCACCTGCATCACCTG′] |
| 4331 | NM_000834.3(GRIN2B): | 2904 | GRIN2B | [′GGATTTGTTTGCTTTTTTCCTGTACRGGAA |
| | c.2172 − 2A > G | | | ACTGGATGCCTTCATCTATGA′] |
| 4332 | NM_018486.2(HDAC8): c.539A > G | 55869 | HDAC8 | [′ATTCTCTACGTGGATTTGGATCTGCRCCAT |
| | (p.His180Arg) | | | GGAGATGGTAAGCCCTTTGGT′] |
| 4333 | NM_018486.2(HDAC8): | 55869 | HDAC8 | [′ACACTATCCTCTGAGATCCCAGATCRTGAG |
| | c.1001A > G (p.His334Arg) | | | GTAAGTAAGGCTCTGACAAGT′] |
| 4334 | NM_178012.4(TUBB2B): | 347733 | TUBB2B | [′GCAGACCTGCGCAAGCTGGCGGTGARCAT |
| | c.767A > G (p.Asn256Ser) | | | GGTGCCCTTCCCTCGCCTGCAC′] |
| 4335 | NM_006888.4(CALM1): c.293A > G | 801 | CALM1 | [′ATTGCTGAATGTTCACAGGATGGCARTGGT |
| | (p.Asn98Ser) | | | TATATCAGTGCAGCAGAACTA′] |
| 4336 | NM_172107.2(KCNQ2): | 3785 | KCNQ2 | [′GCCCCACCCACCCCCCTGCAGTGTCRTGCG |
| | c.1636A > G (p.Met546Val) | | | GTTCCTGGTGTCCAAGCGGAA′] |
| 4337 | NM_001866.2(COX7B): | 1349 | COX7B | [′TGTATTCTTTTTTCGTTTTCCTGTARGTTCG |
| | c.41 − 2A > G | | | AAGCATTCAGCAAACAATGG′] |
| 4338 | NM_001099922.2(ALG13): | 79868 | ALG13 | [′TGAACAATCATCAGCTGGAACTGGCRAAG |
| | c.339A > G (p.Ala113=) | | | CAGCTACACAAAGAGGGTCATC′] |
| 4339 | NM_001083614.1(EARS2): | 124454 | EARS2 | [′CCCCATCAGGTATGACAATCGGTGCRGGA |
| | c.502A > G (p.Arg168Gly) | | | ACATGAGCCAGGAGCAGGTGGC′] |
| 4340 | NM_001083614.1(EARS2): | 124454 | EARS2 | [′TGCCTTGTACAACTACATCTTTGCTRAGAA |
| | c.193A > G (p.Lys65Glu) | | | GTACCAGGGGAGCTTCATCCT′] |
| 4341 | NM_033109.4(PNPT1): | 87178 | PNPT1 | [′ACCCTTCATGGATCAGCATTATTTCRAAGA |
| | c.1160A > G (p.Gln387Arg) | | | GGACAAACACAGGTAATTTAT′] |
| 4342 | NM_033109.4(PNPT1): | 87178 | PNPT1 | [′CCTTTCACCATAAGAGTTACATCTGRAGTC |
| | c.1424A > G (p.Glu475Gly) | | | CTAGAGTCAAATGGTATGGTA′] |
| 4343 | NM_000390.2(CHM): c.1520A > G | 1121 | CHM | [′ATTTTTTTTTTAACAGATTTGGTTCRTTTGA |
| | (p.His507Arg) | | | CTTGCACATCTTCTAAAACA′] |
| 4344 | NM_181690.2(AKT3): c.686A > G | 10000 | AKT3 | [′TTGTGTTTTGTGATGGAATATGTTARTGGG |
| | (p.Asn229Ser) | | | GGCGAGGTGAGTCAAGAAGTA′] |
| 4345 | NM_006567.3(FARS2): c.431A > G | 10667 | FARS2 | [′CCCAGCAGGAAGAAGGGGGACAACTRTTA |
| | (p.Tyr144Cys) | | | CCTGAATCGGACTCACATGCTG′] |
| 4346 | NM_001410.2(MEGF8): | 1954 | MEGF8 | [′CAAGTGCCGGGAATCATTTCACGGGRGTC |
| | c.7099A > G (p.Ser2367Gly) | | | CGCTGGGCGGCCAGCAGTGCTA′] |
| 4347 | NM_003124.4(SPR): | 6697 | SPR | [′CCTCATCGTCTCCTTTTCATCCTCTRGGTCC |
| | c.596 − 2A > G | | | TCTGGACACAGACATGCAGC′] |
| 4348 | NM_005609.2(PYGM): c.152A > G | 5837 | PYGM | [′GACCGCAATGTGGCCACCCCACGAGRCTA |
| | (p.Asp51Gly) | | | CTACTTTGCTCTGGCCCATACC′] |
| 4349 | NM_004247.3(EFTUD2): | 9343 | EFTUD2 | [′TTACTTAGTATTTGCTTTTCAGGACRTGTG |
| | Gc.623A > (p.His208Arg) | | | AATTTCTCTGATGAGGTCACA′] |
| 4350 | NM_001310338.1(MGME1): | 92667 | MGME1 | [′GCTGTTCAACATGAAACCTTAAACTRTATA |
| | c.743A > G (p.Tyr248Cys) | | | GGTCTGCTGGACTGTGTGGCT′] |
| 4351 | NM_001128085.1(ASPA): | −1 | — | [′AAGAAAGACGTTTTTGATTTTTTTCRGACT |
| | c.433 − 2A > G | | | TCTCTGGCTCCACTACCCTGC′] |
| 4352 | NM_000108.4(DLD): c.1444A > G | 1738 | DLD | [′TGGAGCATCCTGTGAAGATATAGCTRGAG |
| | (p.Arg482Gly) | | | TCTGTCATGCACATCCGGTAAT′] |
| 4353 | NM_007215.3(POLG2): | 11232 | POLG2 | [′ACTAACATTAAGAACAAACAAACCCNTAT |
| | c.1105A > G (p.Arg369Gly) | | | TTTTAGTTTCCCAAGTCTATCT′] |
| 4354 | NM_004333.4(BRAF): c.2126A > G | 673 | BRAF | [′AGAGATGAGAGACCACTCTTTCCCCRAGT |
| | (p.Gln709Arg) | | | AAGTAAAAGCTTCATGCTATCC′] |
| 4355 | NM_004985.4(KRAS): c.65A > G | 3845 | KRAS | [′GTAGGCAAGAGTGCCTTGACGATACDGCT |
| | (p.Gln22Arg) | | | AATTCAGAATCATTTTGTGGAC′] |
| 4356 | NM_004985.4(KRAS): c.458A > G | 3845 | KRAS | [′ACTTTTTATGTATTTCAGGGTGTTGDTGAT |
| | (p.Asp153Gly) | | | GCCTTCTATACATTAGTTCGA′] |
| 4357 | NM_002834.3(PTPN11): | 5781 | PTPN11 | [′TAGTAAAAGTAACCCTGGAGACTTCRCAC |
| | c.124A > G (p.Thr42Ala) | | | TTTCCGTTAGGTAAGTTGGAAT′] |
| 4358 | NM_002834.3(PTPN11): | 5781 | PTPN11 | [′AGCTGTCACCCACATCAAGATTCAGNACA |
| | c.172A > G (p.Asn58Asp) | | | CTGGTGATTACTATGACCTGTA′] |
| 4359 | NM_002834.3(PTPN11): | 5781 | PTPN11 | [′TTTCTTTCTTTCCAGACACTACAACRACAG |
| | c.767A > G (p.Gln256Arg) | | | GAGTGCAAACTTCTCTACAGC′] |
| 4360 | NM_002834.3(PTPN11): | 5781 | PTPN11 | [′CAAAAACAAAAATAGATATAAAAACRTCC |
| | c.844A > G (p.Ile282Val) | | | TGCCCTGTAAGTATCAATATTC′] |
| 4361 | NM_002834.3(PTPN11): | 5781 | PTPN11 | [′GATGGTGCGGTCTCAGAGGTCAGGGRTGG |
| | c.1510A > G (p.Met504Val) | | | TCCAGACAGAAGCACAGTACCG′] |
| 4362 | NM_002880.3(RAF1): c.524A > G | 5894 | RAF1 | [′ACTTGTGGCTACAAATTTCATGAGCRCTGT |
| | (p.His175Arg) | | | AGCACCAAAGTACCTACTATG′] |
| 4363 | NM_002880.3(RAF1): c.1279A > G | 5894 | RAF1 | [′AATTGTGACCCAGTGGTGCGAGGGCRGCA |
| | (p.Ser427Gly) | | | GCCTCTACAAACACCTGCATGT′] |
| 4364 | NM_005633.3(SOS1): c.508A > G | 6654 | SOS1 | [′TATTAAAGTGGCAATGTGTGCTGACRAGG |
| | (p.Lys170Glu) | | | TAGGAAACTGAGCTTTTCTATT′] |
| 4365 | NM_030662.3(MAP2K2): | 5605 | MAP2K2 | [′GCGGCTGGAAGCCTTTCTCACCCAGRAAG |
| | c.181A > G (p.Lys61Glu) | | | CCAAGGTCGGCGAACTCAAAGA′] |
| 4366 | NM_024334.2(TMEM43): | 79188 | TMEM43 | [′GGAGAATGAAGGAAGGCTGGTGCACRTCA |
| | c.271A > G (p.Ile91Val) | | | TTGGCGCCTTACGGACATCCAA′] |
| 4367 | NM_000084.4(CLCN5): | 1184 | CLCN5 | [′CTGATGGCTGCAGCCATGACAAGCARGTG |
| | c.1637A > G (p.Lys546Arg) | | | GGTGGCAGATGCTCTTGGGCGG′] |
| 4368 | NM_000084.4(CLCN5): c.815A > G | 1184 | CLCN5 | [′CCTTCTTTCTTCTAGGTCAGCTACTRTTTTC |
| | (p.Tyr272Cys) | | | CCCTCAAAACATTGTGGCGT′] |
| 4369 | NM_000095.2(COMP): c.1760A > G | 1311 | COMP | [′GGCGTGGACTTCGAGGGCACGTTCCRTGT |
| | (p.His587Arg) | | | GAACACGGTCACGGATGACGAC′] |
| 4370 | NM_000530.6(MPZ): c.389A > G | 4359 | MPZ | [′AATGGCACGTTCACTTGTGACGTCARAAA |
| | (p.Lys130Arg) | | | CCCTCCAGACATAGTGGGCAAG′] |
| 4371 | NM_000530.6(MPZ): c.347A > G | 4359 | MPZ | [′AAGGATGGCTCCATTGTCATACACARCCTA |
| | (p.Asn116Ser) | | | GACTACAGTGACAATGGCACG′] |
| 4372 | NM_003611.2(OFD1): c.260A > G | 8481 | OFD1 | [′GCAGATCACTTACAAAGATGTGGCTDTGA |
| | (p.Tyr87Cys) | | | ATATTCACTTTCTGTTTTCTTT′] |
| 4373 | NM_003611.2(OFD1): c.290A > G | 8481 | OFD1 | [′TATTCACTTTCTGTTTTCTTTCCAGRAAGTG |
| | (p.Glu97Gly) | | | GTTTGGCAAAAGAAAAGGTA′] |
| 4374 | NM_003611.2(OFD1): | 8481 | OFD1 | [′TAAAAGTGAAATATTTTCTTTTAACRGGTT |
| | c.382 − 2A > G | | | TCAGGATCTGATAAAGAAAAT′] |
| 4375 | NM_003977.3(AIP): c.721A > G | 9049 | AIP | [′GCTGCTGCTCAACTACTGCCAGTGCDAGCT |
| | (p.Lys241Glu) | | | GGTGGTCGAGGAGTACTACGA′] |
| 4376 | NM_006158.4(NEFL): c.293A > G | 4747 | NEFL | [′GAGAAGGCGCAGCTCCAGGACCTCARTGA |
| | (p.Asn98Ser) | | | CCGCTTCGCCAGCTTCATCGAG′] |
| 4377 | NM_024312.4(GNPTAB): | 79158 | GNPTAB | [′ATATCTCAAGTCTTTGATGAAGTTGRTACA |
| | c.3053A > G (p.Asp1018Gly) | | | GATCAATCTGGTGTCTTGTCT′] |
| 4378 | NM_025137.3(SPG11): | 80208 | SPG11 | [′TAAAGCTAACTTTTATTTTTCCTATRGAGA |
| | c.1457 − 2A > G | | | ATGGACTCTCTCTGATTTTGT′] |
| 4379 | NM_025137.3(SPG11): | 80208 | SPG11 | [′ACAAGAATCCATCCTTCTCCCCAGGRTAAG |
| | c.2608A > G (p.Ile870Val) | | | TCCAGAAGGCAAGTGTGAGAG′] |
| 4380 | NM_025137.3(SPG11): | 80208 | SPG11 | [′GAATGAAATTTTAGATAAGCTGGCCRGGT |
| | c.2833A > G (p.Arg945Gly) | | | ATTATAACTGTTGAACTAATAC′] |
| 4381 | NM_025137.3(SPG11): | 80208 | SPG11 | [′CAGTGAGGAGTATGGGCTGGTGGTARGTA |
| | c.6477 + 4A > G | | | GCCCCCTCAACCCCAGTCTCCA′] |
| 4382 | NM_212472.2(PRKARIA): | 5573 | PRKARIA | [′ATTTTGCAAACTCGTAATTTCTTTCRGGAG |
| | c.178 − 2A > G | | | GAGGCAAAACAGATTCAGAAT′] |
| 4383 | NM_006231.3(POLE): | 5426 | POLE | [′CCAGTTCAGCTACCTGGAACCAGGTRTGG |
| | c.4444 + 3A > G | | | CCTGCACCAGCCGCCCATCATG′] |
| 4384 | NM_003156.3(STIM1): c.251A > G | 6786 | STIM1 | [′GACGATGATGCCAATGGTGATGTGGRTGT |
| | (p.Asp84Gly) | | | GGAAGAAAGTGATGAGGTGAGC′] |
| 4385 | NM_003156.3(STIM1): c.326A > G | 6786 | STIM1 | [′CCAACAGTGAAACACAGCACCTTCCRTGG |
| | (p.His 109Arg) | | | TGAGGATAAGCTCATCAGCGTG′] |
| 4386 | NM_001128425.1(MUTYH): | 4595 | MUTYH | [′GGCTAAGAGCTGTTCCTGCTCCACCYGAG |
| | c.934 − 2A > G | | | AGGCACAGGGTTGAGTGTCATA′] |
| 4387 | NM_133499.2(SYN1): c.1699A > G | 6853 | SYN1 | [′GGCGGAGCCGGGCCAGAGACGGATGYCTG |
| | (p.Thr567Ala) | | | ACGGGTAGCCTGTGGGGGGCCC′] |
| 4388 | NM_021629.3(GNB4): c.265A > G | 59345 | GNB4 | [′TATTTGGGATAGCTATACAACAAATRAGG |
| | (p.Lys89Glu) | | | TAGAATTTCTTCATAATTCTTT′] |
| 4389 | NM_138425.3(C12orf57): | 113246 | C12orf57 | [′CTGAACCTAGAGCTTCAGACGCCCTRTGGC |
| | c.1A > G (p.Met1Val) | | | GTCCGCCTCGACCCAACCGGC′] |
| 4390 | NM_000096.3(CP): c.2953A > G | 1356 | CP | [′GGGAGATGAAGTCAACTGGTATCTGRTGG |
| | (p.Met985Val) | | | GAATGGGCAATGAAATAGACTT′] |
| 4391 | NM_000057.3(BLM): | 641 | BLM | [′AATATTAACAACATAATTATTTTATRGCTA |
| | c.1088 − 2A > G | | | GACAGATAAGTTTACAGCAGC′] |
| 4392 | NM_000257.3(MYH7): c.5807A > G | 4625 | MYH7 | [′TTTCAAAAGGGCTTGAATGAGGAGTRGCT |
| | (p.Ter1936Trp) | | | TTGCCACATCTTGATCTGCTCA′] |
| 4393 | NM_152263.3(TPM3): c.505A > G | 7170 | TPM3 | [′GGATCTTTTCCTGTAGGTGGCTCGTRAGTT |
| | (p.Lys169Glu) | | | GGTGATCATTGAAGGAGACTT′] |
| 4394 | NM_152263.3(TPM3): c.733A > G | 7170 | TPM3 | [′AGAGACCCGTGCTGAGTTTGCTGAGRGAT |
| | (p.Arg245Gly) | | | CGGTAGCCAAGCTGGAAAAGAC′] |
| 4395 | NM_000096.3(CP): | 1356 | CP | [′GCATTAAACACTTTTTTCCCCCTGCRGTGA |
| | c.1209 − 2A > G | | | CTCAGCGGTGTTTTTTGAACA′] |
| 4396 | NM_002739.3(PRKCG): c.76A > G | 5582 | PRKCG | [′CCTGTTTTGCAGAAAGGGGGCCCTGRGGC |
| | (p.Arg26Gly) | | | AGAAGGTGGTCCACGAAGTCAA′] |
| 4397 | NM_000138.4(FBN1): | 2200 | FBN1 | [′GTGTTTTGTTTTGTTGTGTTTTTCTRGAGGA |
| | c.1148 − 2A > G | | | TTTCAACAAGCTGTGCTCTG′] |
| 4398 | NM_000138.4(FBN1): c.3058A > G | 2200 | FBN1 | [′ACCCGGATTTGCCACAAAAGAAATTRCAA |
| | (p.Thr1020Ala) | | | ATGGAAAGCCTTTCTTCAAAGG′] |
| 4399 | NM_000169.2(GLA): c.1153A > G | −1 | — | [′GGCCTGTAATCCTGCCTGCTTCATCRCACA |
| | (p.Thr385Ala) | | | GCTCCTCCCTGTGAAAAGGAA′] |
| 4400 | NM_000256.3(MYBPC3): | 4607 | MYBPC3 | [′ACTTCAACGGCCCCTTCTGTTCTACRGCAA |
| | c.1224 − 2A > G | | | GTAAGTTCCCCTCTGGATGGC′] |
| 4401 | NM_000256.3(MYBPC3): | 4607 | MYBPC3 | [′GTCAGCCTCGTCGGCAGGTGTGACGYCGT |
| | c.1814A > G (p.Asp605Gly) | | | CAATGGTCAGTTTGTGGACCCT′] |
| 4402 | NM_000256.3(MYBPC3): | 4607 | MYBPC3 | [′CTCTGAACTACATTGTGTCTTCTGCRGAAC |
| | c.1928 − 2A > G | | | CTCCCAAGATCCACCTGGACT′] |
| 4403 | NM_000256.3(MYBPC3): | 4607 | MYBPC3 | [′GGGCCGCAGGTGCGTCTGGCACGTCBGGA |
| | c.2309 − 2A > G | | | TGGGGTGGGATGGACCCACATC′] |
| 4404 | NM_000256.3(MYBPC3): | 4607 | MYBPC3 | [′GTGGCTTCTTGCTAAAAGCTGAGACYGAA |
| | c.26 − 2A > G | | | GGGCCAGGTGGAGGCTACAGCG′] |
| 4405 | NM_000256.3(MYBPC3): | 4607 | MYBPC3 | [′GGCCACAGCCTAGACTGCGGGACACRGGG |
| | c.927 − 2A > G | | | ACTCGAAGCTGGAGGCACCAGC′] |
| 4406 | NM_000257.3(MYH7): c.2206A > G | 4625 | MYH7 | [′AGCGGCCATCCCTGAGGGACAGTTCRTTG |
| | (p.Ile736Val) | | | ATAGCAGGAAGGGGGCAGAGAA′] |
| 4407 | NM_000257.3(MYH7): c.2681A > G | 4625 | MYH7 | [′CTCCCCTCTGTTCCTCACCTTCAGGDACAA |
| | (p.Glu894Gly) | | | GACAACCTGGCAGATGCTGAG′] |
| 4408 | NM_000260.3(MYO7A): | 4647 | MYO7A | [′CCTTACCCCATCCCTGTGCCCCTGCRGCTT |
| | c.1344 − 2A > G | | | TGAGCAGCTCTGCATCAACTT′] |
| 4409 | NM_000260.3(MYO7A): | 4647 | MYO7A | [′GTGCCAGGGAAGGATCCCATGGCCGRTTC |
| | c.6029A > G (p.Asp2010Gly) | | | CATCTTCCACTATTACCAGGTG′] |
| 4410 | NM_000260.3(MYO7A): | 4647 | MYO7A | [′CTCTCTATGCCCTTTCTGCTCCCCCRGGAT |
| | c.6439 − 2A > G | | | ATCCTCACCACTCATCCCTTC′] |
| 4411 | NM_000441.1(SLC26A4): | −1 | — | [′CCCTCCTCGCTGTCCTCTGGCTCGCRGGTC |
| | c.−3 − 2A > G | | | ATGGCAGCGCCAGGCGGCAGG′] |
| 4412 | NM_000441.1(SLC26A4): | 5172 | SLC26A4 | [′ATTACACCATCGATGGGAACCAGGTDTGG |
| | c.1149 + 3A > G | | | GTGCCCTTTTGCTGAACTGGTT′] |
| 4413 | NM_000551.3(VHL): c.467A > G | 7428 | VHL | [′GATTTGGTTTTTGCCCTTCCAGTGTRTACTC |
| | (p.Tyr156Cys) | | | TGAAAGAGCGATGCCTCCAG′] |
| 4414 | NM_002294.2(LAMP2): | 3920 | LAMP2 | [′AAATATTATTTTTTTAAATGAATCCRGGAG |
| | c.65 − 2A > G | | | CTGTGCGGTCTTATGCATTGG′] |
| 4415 | NM_004004.5(GJB2): c.1A > G | 2706 | GJB2 | [′CAGAGCAAACCGCCCAGAGTAGAAGRTGG |
| | (p.Met1Val) | | | ATTGGGGCACGCTGCAGACGAT′] |
| 4416 | NM_004004.5(GJB2): c.617A > G | 2706 | GJB2 | [′GTGTCTGGAATTTGCATCCTGCTGARTGTC |
| | (p.Asn206Ser) | | | ACTGAATTGTGTTATTTGCTA′] |
| 4417 | NM_033360.3(KRAS): c.182A > G | 3845 | KRAS | [′TTGGATATTCTCGACACAGCAGGTCNAGA |
| | (p.Gln61Arg) | | | GGAGTACAGTGCAATGAGGGAC′] |
| 4418 | NM_006218.2(PIK3CA): | 5290 | PIK3CA | [′GATCCTCTCTCTGAAATCACTGAGCRGGAG |
| | c.1637A > G (p.Gln546Arg) | | | AAAGATTTTCTATGGAGTCAC′] |
| 4419 | NM_006218.2(PIK3CA) | 5290 | PIK3CA | [′TGATGACATTGCATACATTCGAAAGRCCCT |
| | c.3073A > G (p.Thr1025Ala) | | | AGCCTTAGATAAAACTGAGCA′] |
| 4420 | NM_032119.3(ADGRV1): | 84059 | ADGRV1 | [′TTCTTCATGATTTAATTTTTTTCCCRGATCA |
| | c.14973 − 2A > G | | | GGTTTCATTGTTGCTGAAAT′] |
| 4421 | NM_033056.3(PCDH15): | 65217 | PCDH15 | [′TTATTTGTTTGTTTGTTTTGTCACTRGCACG |
| | c.1998 − 2A > G | | | GGGATTCTAACCTTAGGGAA′] |
| 4422 | NM_138691.2(TMC1): | 117531 | TMC1 | [′ATCTTCAACCAAGGCATGATCTGGTRGGCC |
| | c.1763 + 3A > G | | | AGCTGTTGGACAGCTTATCAC′] |
| 4423 | NM_206933.2(USH2A): | 7399 | USH2A | [′CAACTTAACCTGTTAATTTTCTTACRGGGA |
| | c.12067 − 2A > G | | | ACAAGCCATCAAGCCCACCTG′] |
| 4424 | NM_206933.2(USH2A): | 7399 | USH2A | [′CCCGTCACTGAAGATGTTGTATGTCYACAG |
| | c.12295 − 2A > G | | | AAGGACAGAAGCAAAAGGGAT′] |
| 4425 | NM_206933.2(USH2A): | 7399 | USH2A | [′ATAATGCATAACCTTTCCCTGATGCRGGAA |
| | c.1841 − 2A > G | | | GGAACTGTGAGCTGTGCAAGG′] |
| 4426 | NM_206933.2(USH2A): | 7399 | USH2A | [′GATGTTCCTGCTTGTCTTTTGCTTTRGATAT |
| | c.8559 − 2A > G | | | GAGCTTCTGAGACGTAAAAT′] |
| 4427 | NM_032415.5(CARD11): | 84433 | CARD11 | [′GGCCTCACGCACTTCCTGATGAACGRGGTC |
| | c.401A > G (p.Glu134Gly) | | | ATCAAGCTGCAGCAGCAGATG′] |
| 4428 | NM_000548.3(TSC2): | 7249 | TSC2 | [′CACCGCTGTCCCCTCTGCTGGTGACRGCAC |
| | c.226 − 2A > G | | | GCAGTGGAAGCACTCTGGAAG′] |
| 4429 | NM_000548.3(TSC2): | 7249 | TSC2 | [′TCATTGGCCTCCCTTGTGCCTGTGCRGGAG |
| | c.1444 − 2A > G | | | GAGCTGATTAACTCAGTGGTC′] |
| 4430 | NM_000548.3(TSC2): | 7249 | TSC2 | [′CCCTGACCACCCTCTCCATTACCGCDGCTC |
| | c.2546 − 2A > G | | | TGGCCAGGCTGCCGCACCTCT′] |
| 4431 | NM_130466.3(UBE3B): | 89910 | UBE3B | [′TTTGTTCTCACTGTTTTCTTTCTTTRGGTGA |
| | c.545 − 2A > G | | | AAGTCTTCGACCAGCGATGA′] |
| 4432 | NM_005430.3(WNT1): | 7471 | WNT1 | [′CAACAACGAGGCAGGCCGTACGGTGRGCT |
| | c.624 + 4A > G | | | TTGAGAGGCTCCGCACCCTAAG′] |
| 4433 | NM_001893.4(CSNK1D): | 1453 | CSNK1D | [′AAGCTTGAATGTGTCAAAACCAAACRCCC |
| | c.137A > G (p.His46Arg) | | | TCAGCTCCACATTGAGAGCAAA′] |
| 4434 | NM_018972.2(GDAP1): c.368A > G | 54332 | GDAP1 | [′AGCATGTATTACCCACGGGTACAACRTTAC |
| | (p.His123Arg) | | | CGAGAGCTGCTTGACTCCTTG′] |
| 4435 | NM_032237.4(POMK): c.773A > G | 84197 | POMK | [′CATGGGGATTTCGTGGCTCCAGAGCRACT |
| | (p.Gln258Arg) | | | GTGGCCCTATGGAGAGGACGTG′] |
| 4436 | NM_201647.2(STAMBP): | 10617 | STAMBP | [′CGTCGGTACTTCCGCTCTGGAGTTGRGATT |
| | c.125A > G (p.Glu42Gly) | | | ATCCGAATGGCATCCATTTAC′] |
| 4437 | NM_001946.3(DUSP6): c.566A > G | 1848 | DUSP6 | [′ACTACCATCCGAGTCTGTTGCACTAYTGGG |
| | (p.Asn189Ser) | | | GTCTCGGTCAAGGTCAGACTC′] |
| 4438 | NM_003867.3(FGF17): c.560A > G | 8822 | FGF17 | [′TACCAAGGCCAGCTGCCCTTCCCCARCCAC |
| | (p.Asn187Ser) | | | GCCGAGAAGCAGAAGCAGTTC′] |
| 4439 | NM_013281.3(FLRT3): | −1 | — | [′GGGCTCATGTGCCAAGCCCCAGAAARGGT |
| | c.1016A > G (p.Lys339Arg) | | | TCGTGGGATGGCTATTAAGGAT′] |
| 4440 | NM_015560.2(OPA1): c.1146A > G | 4976 | OPA1 | [′TTTTTATTTTTCCTGAGTAGACCATRTCCTT |
| | (p.Ile382Met) | | | AAATGTAAAAGGCCCTGGAC′] |
| 4441 | NM_030964.3(SPRY4): c.530A > G | 81848 | SPRY4 | [′CCGGCGGTCCCACCCGAGCTGGACARGCA |
| | (p.Lys177Arg) | | | CTTCTTGCTGTGCGAGGCCTGT′] |
| 4442 | NM_002972.3(SBF1): c.1249A > G | 6305 | SBF1 | [′AAGGCCATGCCCTCCAGCACCTTCAYCAG |
| | (p.Met417Val) | | | GAAATCGTCCTCTACCAGCCCA′] |
| 4443 | NM_002972.3(SBF1): c.4768A > G | 6305 | SBF1 | [′TACATGTAATTGTGGAACACAGGCGYCCT |
| | (p.Thr1590Ala) | | | CTTGCTCAGCCGGTCCACATAC′] |
| 4444 | NM_006876.2(B4GAT1): | 11041 | B4GAT1 | [′GTTCCATCCCCAAAAGGAGGCTGAARATC |
| | c.1168A > G (p.Asn390Asp) | | | AGCACAATAAGATCCTATATCG′] |
| 4445 | NM_000059.3(BRCA2): | 675 | BRCA2 | [′AAAATAAACTATTTTCTTTCCTCCCVGGGT |
| | c.517 − 2A > G | | | CGTCAGACACCAAAACATATT′] |
| 4446 | NM_000059.3(BRCA2): | 675 | BRCA2 | [′ATGTAATATAAAATAATTGTTTCCTRGGCA |
| | c.6938 − 2A > G | | | CAATAAAAGATCGAAGATTGT′] |
| 4447 | NM_000059.3(BRCA2): | 675 | BRCA2 | [′AACTTATATATTTTCTCCCCATTGCDGCAC |
| | 2c.7008 − A > G | | | AACTAAGGAACGTCAAGAGAT′] |
| 4448 | NM_000059.3(BRCA2): | 675 | BRCA2 | [′GATAATATTCTACTTTTATTTGTTCRGGGC |
| | c.7806 − 2A > G | | | TCTGTGTGACACTCCAGGTGT′] |
| 4449 | NM_000059.3(BRCA2): | 675 | BRCA2 | [′AGCAGACCCAGCTTACCTTGAGGTGRGAG |
| | c.8754 + 4A > G | | | AGTAAGAGGACATATAATGAGG′] |
| 4450 | NM_000059.3(BRCA2): | 675 | BRCA2 | [′GTTGAATTTTTGTTTTGTTTTCTGTRGGTTT |
| | c.9118 − 2A > G | | | CAGATGAAATTTTATTTCAG′] |
| 4451 | NM_000059.3(BRCA2): | 675 | BRCA2 | [′TAGGCTACGTTTTCATTTTTTTATCRGATGT |
| | c.9649 − 2A > G | | | CTTCTCCTAATTGTGAGATA′] |
| | NM_000218.2(KCNQ1): | 3784 | KCNQ1 | [] |
| | c.1070A > G (p.Gln357Arg) | | | |
| 4452 | NM_000218.2(KCNQ1): | 3784 | KCNQ1 | [′AAGGTGCAGCAGAAGCAGAGGCAGARGC |
| | Gc.1085A > (p.Lys362Arg) | | | ACTTCAACCGGCAGATCCCGGCG′] |
| 4453 | NM_000218.2(KCNQ1): | 3784 | KCNQ1 | [′TCGACGCATGCAGTACTTTGTGGCCVAGA |
| | c.1576A > G (p.Lys526Glu) | | | AGAAATTCCAGGTAAGCCCTGT′] |
| | NM_000218.2(KCNQ1): | 3784 | KCNQ1 | |
| | c.1756A > G (p.Asn586Asp) | | | |
| 4454 | NM_000218.2(KCNQ1): c.332A > G | 3784 | KCNQ1 | [′CGCACCCACGTCCAGGGCCGCGTCTRCAA |
| | (p.Tyr111Cys) | | | CTTCCTCGAGCGTCCCACCGGC′] |
| | NM_000218.2(KCNQ1): c.344A > G | 3784 | KCNQ1 | [] |
| | (p.Glu115Gly) | | | |
| 4455 | NM_000218.2(KCNQ1): c.773A > G | 3784 | KCNQ1 | [′CTCCTGGGCTCCGTGGTCTTCATCCVCCGC |
| | (p.His258Arg) | | | CAGGTGGGTGGCCCGGGTTAG′] |
| 4456 | NM_000218.2(KCNQ1): c.944A > G | 3784 | KCNQ1 | [′CAGGTCACAGTCACCACCATCGGCTNTGG |
| | (p.Tyr315Cys) | | | GGACAAGGTGCCCCAGACGTGG′] |
| | NM_000218.2(KCNQ1): c.964A > G | 3784 | KCNQ1 | = |
| | (p.Thr322Ala) | | | |
| 4457 | NM_000492.3(CFTR): | 1080 | CFTR | [′CCTAATAATGATGGGTTTTATTTCCRGACT |
| | c.1393 − 2A > G | | | TCACTTCTAATGGTGATTATG′] |
| 4458 | NM_000492.3(CFTR): | 1080 | CFTR | [′ACAGAAAAAGAAATATTTGAAAGGTVTGT |
| | c.1766 + 3A > G | | | TCTTTGAATACCTTACTTATAA′] |
| 4459 | NM_000492.3(CFTR): c.1A > G | 1080 | CFTR | [′CAGGGACCCCAGCGCCCGAGAGACCRTGC |
| | (p.Met1Val) | | | AGAGGTCGCCTCTGGAAAAGGC′] |
| 4460 | NM_000492.3(CFTR): | 1080 | CFTR | [′ACCAACATGTTTTCTTTGATCTTACDGTTG |
| | c.2989 − 2A > G | | | TTATTAATTGTGATTGGAGCT′] |
| 4461 | NM_000492.3(CFTR): | 1080 | CFTR | [′ACAACCTGAACAAATTTGATGAAGTNTGT |
| | c.579 + 3A > G | | | ACCTATTGATTTAATCTTTTAG′] |
| 4462 | NM_007294.3(BRCA1): c.122A > G | 672 | BRCA1 | [′GAACCTGTCTCCACAAAGTGTGACCRCAT |
| | (p.His41Arg) | | | ATTTTGCAAGTAAGTTTGAATG′] |
| 4463 | NM_007294.3(BRCA1): c.1A > G | 672 | BRCA1 | [′TTAAAGTTCATTGGAACAGAAAGAARTGG |
| | (p.Met1Val) | | | ATTTATCTGCTCTTCGCGTTGA′] |
| 4464 | NM_007294.3(BRCA1): | 672 | BRCA1 | [′TGTAAGAATGATATAACCAAAAGGTRTAT |
| | c.212 + 3A > G | | | AATTTGGTAATGATGCTAGGTT′] |
| 4465 | NM_007294.3(BRCA1): | 672 | BRCA1 | [′TCTGAACCTCTGTTTTTGTTATTTAVGGTG |
| | c.4097 − 2A > G | | | AAGCAGCATCTGGGTGTGAGA′] |
| 4466 | NM_007294.3(BRCA1): | 672 | BRCA1 | [′GTTTTCTCCTTCCATTTATCTTTCTRGGTCA |
| | c.4485 − 2A > G | | | TCCCCTTCTAAATGCCCATC′] |
| 4467 | NM_007294.3(BRCA1): | 672 | BRCA1 | [′ATAATGGAATATTTGATTTAATTTCRGATG |
| | c.4987 − 2A > G | | | CTCGTGTACAAGTTTGCCAGA′] |
| 4468 | NM_007294.3(BRCA1): | 672 | BRCA1 | [′AACTAATCTAATTACTGAAGAGACTRCTCA |
| | c.5053A > G (p.Thr1685Ala) | | | TGTTGTTATGAAAACAGGTAT′] |
| 4469 | NM_007294.3(BRCA1): | 672 | BRCA1 | [′AATGCTCTTTCCTTCCTGGGGATCCDGGGT |
| | c.5407 − 2A > G | | | GTCCACCCAATTGTGGTTGTG′] |
| 4470 | NM_000540.2(RYR1): c.97A > G | 6261 | RYR1 | [′CGCTACCGTGCTCAAGGAGCAGCTCRAGC |
| | (p.Lys33Glu) | | | TCTGCCTGGCCGCCGAGGGCTT′] |
| 4471 | NM_015250.3(BICD2): | 23299 | BICD2 | [′CGGCAGCTGGCGGCTGCTGAGGACGRGAA |
| | c.2321A > G (p.Glu774Gly) | | | GAAGACGCTGAACTCGCTGCTG′] |
| 4472 | NM_006012.2(CLPP): | 8192 | CLPP | [′CATCGTGTGCGTCATGGGCCCGGTGRGCG |
| | c.270 + 4A > G | | | CCCCGCGCCGGGACCCTCCCCA′] |
| 4473 | NM_004656.3(BAP1): c.277A > G | 8314 | BAP1 | [′AGGAGCACGCTCAGCAAGGCATGAGHTGC |
| | (p.Thr93Ala) | | | ACAAGAGTTGGGTATCAGCTGT′] |
| 4474 | NM_004315.4(ASAH1): | 427 | ASAH1 | [′AGGAATCATTGGATGTATATGAGTARGTA |
| | c.965 + 4A > G | | | GGTTTGTTAAAGCAAAAGAAGT′] |
| 4475 | NM_000100.3(CSTB): | 1476 | CSTB | [′GCTTCGCTCACTCCGCTCTCTTCCCRGGTG |
| | c.169 − 2A > G | | | CACGTCGGCGACGAGGACTTC′] |
| 4476 | NM_015268.3(DNAJC13): | 23317 | DNAJC13 | [′TTAAAATCTAGGTATGAATTTTTCARTGAG |
| | c.2564A > G (p.Asn855Ser) | | | CTTTATCATCGCTTCTTGCTC′] |
| 4477 | NM_001003722.1(GLE1): | 2733 | GLE1 | [′CTTAAAAAAAAAAAAAAAAAAAAAARCCT |
| | c.433 − 10A > G | | | TTTCAGGAGGGCCTGAGGCTAT′] |
| 4478 | NM_001142519.1(FAM111A): | 63901 | FAM111A | [′TTCCAGAAAATAGTTCACAACCCTGDTGTG |
| | c.1583A > G (p.Asp528Gly) | | | ATTACCTATGACACTGAATTT′] |
| 4479 | NM_001142519.1(FAM111A): | 63901 | FAM111A | [′GCATAGAACAACGTTTGGGAAAGTARCAA |
| | c.1012A > G (p.Thr338Ala) | | | AAAATTCTTCTTCGATTAAAGT′] |
| 4480 | NM_001927.3(DES): | 1674 | DES | [′CTGAGTGTGCGATGGACCCTGTTACRGAA |
| | c.1289 − 2A > G | | | ACCAGCCCTGAGCAAAGGGGTT′] |
| 4481 | NM_014795.3(ZEB2): c.3134A > G | 9839 | ZEB2 | [′AAACACAAGCACCACCTTATCGAGCRCTC |
| | (p.His1045Arg) | | | AAGGCTTCACTCGGGCGAGAAG′] |
| 4482 | NM_002163.2(IRF8): c.322A > G | 3394 | IRF8 | [′CCAACTGGACATTTCCGAGCCATACRAAG |
| | (p.Lys108Glu) | | | TTTACCGAATTGTTCCTGAGGA′] |
| 4483 | NM_002163.2(IRF8): c.238A > G | 3394 | IRF8 | [′CAAAGCTGAACCAGCCACTTGGAAGRCGA |
| | (p.Thr80Ala) | | | GGTTACGCTGTGCTTTGAATAA′] |
| 4484 | NM_001287.5(CLCN7): c.296A > G | 1186 | CLCN7 | [′TGTCCCGGCCTGCAGAGCTTGGACTRTGAC |
| | (p.Tyr99Cys) | | | AACAGTGAGAACCAGCTGTTC′] |
| 4485 | NM_001127217.2(SMAD9): | 4093 | SMAD9 | [′AGAGAAGGCAGTGGACTCTCTAGTGRAGA |
| | c.127A > G (p.Lys43Glu) | | | AGTTAAAGAAGAAGAAGGGAGC′] |
| 4486 | NM_002246.2(KCNK3): c.575A > G | 3777 | KCNK3 | [′TGGACCTTCTTCCAGGCCTACTACTRCTGC |
| | (p.Tyr192Cys) | | | TTCATCACCCTCACCACCATC′] |
| 4487 | NM_080605.3(B3GALT6): c.1A > G | 126792 | B3GALT6 | [′CGCCACGCCCGCCGCAGCAGCTTCAYGGC |
| | (p.Met1Val) | | | GCCCGCGCCGGGCCGGCGGCCC′] |
| 4488 | NM_080605.3(B3GALT6): | 126792 | B3GALT6 | [′CGGCGCTCGGCGGCGCGGGGCGCGCYGGC |
| | c.193A > G (p.Ser65Gly) | | | CACCAGCACTGCCAGGAAGGCG′] |
| 4489 | NM_000388.3(CASR): c.85A > G | 846 | CASR | [′CGGGCCAGACCAGCGAGCCCAAAAGRAGG |
| | (p.Lys29Glu) | | | GGGACATTATCCTTGGGGGGCT′] |
| 4490 | NM_003793.3(CTSF): c.962A > G | 8722 | CTSF | [′GGGACCCTGCTCTCCCTCTCTGAACRGGGT |
| | (p.Gln321Arg) | | | GAGCATCTCGCTCTACTCCTC′] |
| 4491 | NM_003793.3(CTSF): c.692A > G | 8722 | CTSF | [′GAGATCACTGAACTTGGTGACTCCAYACT |
| | (p.Tyr231Cys) | | | GAGCTGTGCCACGGTCCAGGGC′] |
| 4492 | NM_022114.3(PRDM16): | 63976 | PRDM16 | [′GGCAGCCGGGCCCGTGCCAGCCAAARCGG |
| | c.2447A > G (p.Asn816Ser) | | | CGGCGGGCGGGAGCCCCGCAAG′] |
| 4493 | NM_173551.4(ANKS6): | 203286 | ANKS6 | [′GGGCAGCACTGGGATGCTCCAGGGCYGTC |
| | c.1322A > G (p.Gln441Arg) | | | GGACCTTCGAGTGGGGCAGGGG′] |
| 4494 | NM_005689.2(ABCB6): c.508A > G | 10058 | ABCB6 | [′GAACTTGGCCCTGGTGTCTTGGAACRGCCC |
| | (p.Ser170Gly) | | | ACAGTGGTGGTGGGCAAGGGC′] |
| 4495 | NM_001070.4(TUBG1): c.275A > G | 7283 | TUBG1 | [′AAGCTCTACAACCCAGAGAACATCTRCCT |
| | (p.Tyr92Cys) | | | GTCGGAACATGGAGGAGGAGCT′] |
| 4496 | NM_001613.2(ACTA2): c.145A > G | 59 | ACTA2 | [′ACCTTTTTAGGGGGTGATGGTGGGARTGG |
| | (p.Met49Val) | | | GACAAAAAGACAGCTACGTGGG′] |
| 4497 | NM_031157.2(HNRNPA1): | 3178 | HNRNPA1 | [′AGCTACAATGATTTTGGGAATTACARCAAT |
| | c.956A > G (p.Asn319Ser) | | | CAGTCTTCAAATTTTGGACCC′] |
| 4498 | NM_020117.9(LARS): c.1118A > G | 51520 | LARS | [′CTTAATAGTTAGCATTGGGAGAACAYAGA |
| | (p.Tyr373Cys) | | | TCACCTTGTATGATGTTAAAGG′] |
| 4499 | NM_001651.3(AQP5): c.367A > G | 362 | AQP5 | [′CTAACCCGCTATCCCCTTGCAGCTCRACAA |
| | (p.Asn123Asp) | | | CAACACAACGCAGGGCCAGGC′] |
| 4500 | m.13637A > G | 4540 | MT-ND5 | [′ATAATTCTTCTCACCCTAACAGGTCRACCT |
| | | | | CGCTTCCCCACCCTTACTAAC′] |
| 4501 | NM_000081.3(LYST): | 1130 | LYST | [′GGGAAGGCTTCTGTTCAAGCGATCARTGTT |
| | c.10127A > G (p.Asn3376Ser) | | | TTTCATCCTGCTGTAAGTGAC′] |
| 4502 | NM_000142.4(FGFR3): | 2261 | FGFR3 | [′CCAGCAGTGGGGGCTCGCGGACGTGNAGG |
| | c.2421A > G p.Ter807Trp) | | | GCCACTGGTCCCCAACAATGTG′] |
| 4503 | NM_000207.2(INS): c.*59A > G | −1 | — | [′TCCTGCACCGAGAGAGATGGAATAARGCC |
| | | | | CTTGAACCAGCCCTGCTGTGCC′] |
| 4504 | NM_000370.3(TTPA): c.191A > G | 7274 | TTPA | [′CTGCGCGCCCGGGATTTCGATCTGGRCCTG |
| | (p.Asp64Gly) | | | GCCTGGCGGGTAAGCGTGCGT′] |
| 4505 | NM_001006657.1(WDR35): | 57539 | WDR35 | [′AAACCTTTACGTGTCAAGAAGCTCTRTGTA |
| | c.2912A > G (p.Tyr971Cys) | | | CTGTCAGCCTTACTTATAGAG′] |
| 4506 | NM_004595.4(SMS): c.443A > G | 6611 | SMS | [′GTATATGACGAAGATTCACCTTATCRAAAT |
| | (p.Gln148Arg) | | | ATAAAAATTCTACACTCGAAG′] |
| 4507 | NM_005259.2(MSTN): c.458A > G | −1 | — | [′AAAATACAATACAATAAAGTAGTAARGGC |
| | (p.Lys153Arg) | | | CCAACTATGGATATATTTGAGA′] |
| 4508 | NM_000784.3(CYP27A1): | 1593 | CYP27A1 | [′TGGGCCCTGTACCACCTCTCAAAGGRCCCT |
| | c.1061A > G (p.Asp354Gly) | | | GAGATCCAGGAGGCCTTGCAC′] |
| 4509 | NM_000784.3(CYP27A1): | 1593 | CYP27A1 | [′TCACTCTATGCCACCTTCCTCCCCARGTGG |
| | c.776A > G (p.Lys259Arg) | | | ACTCGCCCCGTGCTGCCTTTC′] |
| 4510 | NM_000540.2(RYR1): | 6261 | RYR1 | [′ATCCCAACTATCGGGCGGCTGCGCARGAG |
| | c.10100A > G (p.Lys3367Arg) | | | GGCAGGGAAGGTGGTGTCCGAG′] |
| 4511 | NM_000540.2(RYR1): | 6261 | RYR1 | [′CTACCTGTACACCGTGGTGGCCTTCRACTT |
| | c.14572A > G (p.Asn4858Asp) | | | CTTCCGCAAGTTCTACAACAA′] |
| 4512 | NM_000540.2(RYR1): | 6261 | RYR1 | [′CGCGGGTGACGAATACGAGCTCTACRGGG |
| | c.14740A > G (p.Arg4914Gly) | | | TGGTCTTCGACATCACCTTCTT′] |
| 4513 | NM_000540.2(RYR1): | 6261 | RYR1 | [′GCCTTCAACTTCTTCCGCAAGTTCTRCAAC |
| | c.14591A > G (p.Tyr4864Cys) | | | AAGAGCGAGGATGAGGATGAA′] |
| 4514 | NM_002336.2(LRP6): c.1298A > G | 4040 | LRP6 | [′GATCGAATAGAAGTGACAAGGCTCARTGG |
| | (p.Asn433Ser) | | | GACCATGAGGAAGATCTTGATT′] |
| 4515 | NM_001257235.1(ALG13): | 79868 | ALG13 | [′GTTATAAACGAAAAGTTGATGAACARTCA |
| | c.8A > G (p.Asn3Ser) | | | TCAGCTGGAACTGGCAAAGCAG′] |
| 4516 | NM_001015879.1(AURKC): | 6795 | AURKC | [′CAGGGTGACTTTTCTTTGCACCCACRGATA |
| | c.379 − 2A > G | | | ATAGAGGAGTTGGCAGATGCC′] |
| 4517 | NM_012160.4(FBXL4): | 26235 | FBXL4 | [′AATTGTACCAGGTTACAGCAGCTGGRCAT |
| | c.1694A > G (p.Asp565Gly) | | | ATTAGGTAAGGTTACAATATAT′] |
| 4518 | NM_020988.2(GNAO1): c.521A > G | 2775 | GNA01 | [′GCCGACTACCAGCCCACCGAGCAGGRCAT |
| | (p.Asp174Gly) | | | CCTCCGAACCAGGGTCAAAACC′] |
| 4519 | NM_001927.3(DES): c.1024A > G | 1674 | DES | [′GACCTGGGTTCCCCCTCTCCTGCAGRACGA |
| | (p.Asn342Asp) | | | TTCCCTGATGAGGCAGATGCG′] |
| 4520 | NM_001927.3(DES): c.1333A > G | 1674 | DES | [′GGGTTCTGAGGTCCATACCAAGAAGRCGG |
| | (p.Thr445Ala) | | | TGATGATCAAGACCATCGAGAC′] |
| 4521 | NM_001927.3(DES): | 1674 | DES | [′TCCTTAAGAAAGTGCATGAAGAGGTRTAC |
| | c.735 + 3A > G | | | CTTGGCCCCTCTTCCTGGGGTC′] |
| 4522 | NM_002055.4(GFAP): c.1097A > G | 2670 | GFAP | [′GCCCTGGACATCGAGATCGCCACCTRCAG |
| | (p.Tyr366Cys) | | | GAAGCTGCTAGAGGGCGAGGAG′] |
| 4523 | NM_002055.4(GFAP): c.1112A > G | 2670 | GFAP | [′ATCGCCACCTACAGGAAGCTGCTAGDGGG |
| | (p.Glu371Gly) | | | CGAGGAGAACCGGTGAGCCCTC′] |
| 4524 | NM_002055.4(GFAP): c.1121A > G | 2670 | GFAP | [′TACAGGAAGCTGCTAGAGGGCGAGGRGAA |
| | (p.Glu374Gly) | | | CCGGTGAGCCCTCATCACAGCC′] |
| 4525 | NM_002055.4(GFAP): c.230A > G | 2670 | GFAP | [′GAGCGGGCAGAGATGATGGAGCTCARTGA |
| | (p.Asn77Ser) | | | CCGCTTTGCCAGCTACATCGAG′] |
| 4526 | NM_002055.4(GFAP): c.770A > G | 2670 | GFAP | [′AACATGCATGAAGCCGAAGAGTGGTRCCG |
| | (p.Tyr257Cys) | | | CTCCAAGGTAGCCCTGCCTGTG′] |
| 4527 | NM_002055.4(GFAP): c.835A > G | 2670 | GFAP | [′CAACGCGGAGCTGCTCCGCCAGGCCRAGC |
| | (p.Lys279Glu) | | | ACGAAGCCAACGACTACCGGCG′] |
| 4528 | NM_005554.3(KRT6A): c.511A > G | 3853 | KRT6A | [′GGAGCGTGAACAGATCAAGACCCTCDACA |
| | (p.Asn171Asp) | | | ACAAGTTTGCCTCCTTCATCGA′] |
| 4529 | NM_005554.3(KRT6A): c.512A > G | 3853 | KRT6A | [′GAGCGTGAACAGATCAAGACCCTCAVCAA |
| | (p.Asn171Ser) | | | CAAGTTTGCCTCCTTCATCGAC′] |
| 4530 | NM_005557.3(KRT16): c.373A > G | 3868 | KRT16 | [′TGAGAAGGTGACCATGCAGAACCTCRATG |
| | (p.Asn125Asp) | | | ACCGCCTGGCCTCCTACCTGGA′] |
| 4531 | NM_170707.3(LMNA): | 4000 | LMNA | [′TTTTGGTTTCTGTGTCCTTCCTCCAVCCCTT |
| | c.640 − 10A > G | | | CCAGGAGCTGCGTGAGACCA′] |
| | NM_000218.2(KCNQ1): c.1061A > | 3784 | KCNQ1 | [] |
| | G (p.Lys354Arg) | | | |
| | NM_000218.2(KCNQ1): c.1138A > | 3784 | KCNQ1 | [] |
| | G (p.Arg380Gly) | | | |
| | NM_000218.2(KCNQ1): c.1193A > | 3784 | KCNQ1 | [] |
| | G (p.Lys398Arg) | | | |
| | NM_000218.2(KCNQ1): c.1640A > | 3784 | KCNQ1 | [] |
| | G (p.Gln547Arg) | | | |
| | NM_000218.2(KCNQ1): c.1669A > | 3784 | KCNQ1 | [] |
| | G (p.Lys557Glu) | | | |
| | NM_000218.2(KCNQ1): c.1705A > | 3784 | KCNQ1 | [] |
| | G (p.Lys569Glu) | | | |
| | NM_000218.2(KCNQ1): c.1793A > | 3784 | KCNQ1 | [] |
| | G (p.Lys598Arg) | | | |
| | NM_000218.2(KCNQ1): c.430A > G | 3784 | KCNQ1 | [] |
| | (p.Thr144Ala) | | | |
| | NM_000218.2(KCNQ1): c.440A > G | 3784 | KCNQ1 | [] |
| | (p.Gln147Arg) | | | |
| | NM_000218.2(KCNQ1): c.548A > G | 3784 | KCNQ1 | [] |
| | (p.Lys183Arg) | | | |
| 4532 | NM_000218.2(KCNQ1): c.592A > G | 3784 | KCNQ1 | [′GCGGCTGCGCTTTGCCCGGAAGCCCRTTTC |
| | (p.Ile198Val) | | | CATCATCGGTGAGTCATGCCT′] |
| 4533 | NM_000218.2(KCNQ1): c.652A > G | 3784 | KCNQ1 | [′CATGGTGGTCCTCTGCGTGGGCTCCRAGGG |
| | (p.Lys218Glu) | | | GCAGGTGTTTGCCACGTCGGC′] |
| 4534 | NM_000218.2(KCNQ1): c.820A > G | 3784 | KCNQ1 | [′CCTGTACATCGGCTTCCTGGGCCTCRTCTT |
| | (p.Ile274Val) | | | CTCCTCGTACTTTGTGTACCT′] |
| | NM_000218.2(KCNQ1): c.842A > G | 3784 | KCNQ1 | [] |
| | (p.Tyr281Cys) | | | |
| | NM_000218.2(KCNQ1): c.931A > G | 3784 | KCNQ1 | [] |
| | (p.Thr311Ala) | | | |
| | NM_000218.2(KCNQ1): c.950A > G | 3784 | KCNQ1 | [] |
| | (p.Asp317Gly) | | | |
| | NM_000238.3(KCNH2): c.1205A > | 3757 | KCNH2 | [] |
| | G (p.His402Arg) | | | |
| | NM_000238.3(KCNH2): c.1259A > | 3757 | KCNH2 | [] |
| | G (p.Tyr420Cys) | | | |
| 4535 | NM_000238.3(KCNH2): c.1280A > | 3757 | KCNH2 | [′ATCTACACGGCTGTCTTCACACCCTVCTCG |
| | G (p.Tyr427Cys) | | | GCTGCCTTCCTGCTGAAGGAG′] |
| | NM_000238.3(KCNH2): c.128A > G | 3757 | KCNH2 | [] |
| | (p.Tyr43Cys) | | | |
| | NM_000238.3(KCNH2): c.1424A > | 3757 | KCNH2 | [] |
| | G (p.Tyr475Cys) | | | |
| 4536 | NM_000238.3(KCNH2): c.1478A > | 3757 | KCNH2 | [′CACCCCGGCCGCATCGCCGTCCACTNCTTC |
| | G (p.Tyr493Cys) | | | AAGGGCTGGTTCCTCATCGAC′] |
| | NM_000238.3(KCNH2): c.1502A > | 3757 | KCNH2 | [] |
| | G (p.Asp501Gly) | | | |
| 4537 | NM_000238.3(KCNH2): c.1685A > | 3757 | KCNH2 | [′ATGTGCACCTTTGCGCTCATCGCGCVCTGG |
| | G (p.His562Arg) | | | CTAGCCTGCATCTGGTACGCC′] |
| 4538 | NM_000238.3(KCNH2): c.1711A > | 3757 | KCNH2 | [′CTGGCTAGCCTGCATCTGGTACGCCVTCGG |
| | G (p.Ile571Val) | | | CAACATGGAGCAGCCACACAT′] |
| 4539 | NM_000238.3(KCNH2): c.1720A > | 3757 | KCNH2 | [′CTGCATCTGGTACGCCATCGGCAACVTGG |
| | G (p.Met574Val) | | | AGCAGCCACACATGGACTCACG′] |
| | NM_000238.3(KCNH2): c.1724A > | 3757 | KCNH2 | [] |
| | G (p.Glu575Gly) | | | |
| 4540 | NM_000238.3(KCNH2): c.173A > G | 3757 | KCNH2 | [′GAGCTGTGCGGCTACTCGCGGGCCGVGGT |
| | (p.Glu58Gly) | | | GATGCAGCGACCCTGCACCTGC′] |
| | NM_000238.3(KCNH2): c.1747A > | 3757 | KCNH2 | [] |
| | G (p.Ile583Val) | | | |
| | NM_000238.3(KCNH2): c.1762A > | 3757 | KCNH2 | [] |
| | G (p.Asn588Asp) | | | |
| | NM_000238.3(KCNH2): c.1777A > | 3757 | KCNH2 | [] |
| | G (p.Ile593Val) | | | |
| | NM_000238.3(KCNH2): c.1783A > | 3757 | KCNH2 | [] |
| | G (p.Lys595Glu) | | | |
| | NM_000238.3(KCNH2): c.1790A > | 3757 | KCNH2 | [] |
| | G (p.Tyr597Cys) | | | |
| | NM_000238.3(KCNH2): c.1826A > | 3757 | KCNH2 | [] |
| | G (p.Asp609Gly) | | | |
| | NM_000238.3(KCNH2): c.1847A > | 3757 | KCNH2 | [] |
| | G (p.Tyr616Cys) | | | |
| | NM_000238.3(KCNH2): c.1885A > | 3757 | KCNH2 | [] |
| | G (p.Asn629Asp) | | | |
| 4541 | NM_000238.3(KCNH2): c.1886A > | 3757 | KCNH2 | [′AGCCTCACCAGTGTGGGCTTCGGCANCGT |
| | G (p.Asn629Ser) | | | CTCTCCCAACACCAACTCAGAG′] |
| | NM_000238.3(KCNH2): c.1897A > | 3757 | KCNH2 | [] |
| | G (p.Asn633Asp) | | | |
| 4542 | NM_000238.3(KCNH2): c.1898A > | 3757 | KCNH2 | [′GTGGGCTTCGGCAACGTCTCTCCCADCACC |
| | G (p.Asn633Ser) | | | AACTCAGAGAAGATCTTCTCC′] |
| | NM_000238.3(KCNH2): c.1903A > | 3757 | KCNH2 | [] |
| | G (p.Asn635Asp) | | | |
| | NM_000238.3(KCNH2): c.1910A > | 3757 | KCNH2 | [] |
| | G (p.Glu637Gly) | | | |
| | NM_000238.3(KCNH2): c.1912A > | 3757 | KCNH2 | [] |
| | G (p.Lys638Glu) | | | |
| 4543 | NM_000238.3(KCNH2): c.1933A > | 3757 | KCNH2 | [′AGAGAAGATCTTCTCCATCTGCGTCDTGCT |
| | G (p.Met645Val) | | | CATTGGCTGTGAGTGTGCCCA′] |
| | NM_000238.3(KCNH2): c.209A > G | 3757 | KCNH2 | [] |
| | (p.His70Arg) | | | |
| 4544 | NM_000238.3(KCNH2): c.2131A > | 3757 | KCNH2 | [′GCACGCCTGGTCCTACACCAACGGCRTCG |
| | G (p.Ile711Val) | | | ACATGAACGCGGTGAGGCCACC′] |
| | NM_000238.3(KCNH2): c.2266A > | 3757 | KCNH2 | [] |
| | G (p.Met756Val) | | | |
| | NM_000238.3(KCNH2): c.2510A > | 3757 | KCNH2 | [] |
| | G (p.Asp837Gly) | | | |
| | NM_000238.3(KCNH2): c.2591A > | 3757 | KCNH2 | [] |
| | G (p.Asp864Gly) | | | |
| | NM_000238.3(KCNH2): c.286A > G | 3757 | KCNH2 | [] |
| | (p.Ile96Val) | | | |
| 4545 | NM_000238.3(KCNH2): c.301A > G | 3757 | KCNH2 | [′CAAAGTGGAAATCGCCTTCTACCGGDAAG |
| | (p.Lys101Glu) | | | ATGGTAGGAGCGGGCCGGGGCG′] |
| 4546 | NM_000238.3(KCNH2): c.3118A > | 3757 | KCNH2 | [′TCGGCGGCCCCGGGGCGACGTGGAGRGCA |
| | G (p.Ser1040Gly) | | | GGCTGGATGCCCTCCAGCGCCA′] |
| | NM_000238.3(KCNH2): c.3233A > | 3757 | KCNH2 | [] |
| | G (p.Tyr1078Cys) | | | |
| | NM_000238.3(KCNH2): c.3343A > | 3757 | KCNH2 | [] |
| | G (p.Met1115Val) | | | |
| | NM_000238.3(KCNH2): c.652A > G | 3757 | KCNH2 | [] |
| | (p.Met218Val) | | | |
| | NM_000238.3(KCNH2): c.82A > G | 3757 | KCNH2 | [] |
| | (p.Lys28Glu) | | | |
| | NM_000891.2(KCNJ2): c.220A > G | 3759 | KCNJ2 | [] |
| | (p.Thr74Ala) | | | |
| | NM_000891.2(KCNJ2): c.223A > G | 3759 | KCNJ2 | [] |
| | (p.Thr75Ala) | | | |
| | NM_000891.2(KCNJ2): c.233A > G | 3759 | KCNJ2 | [] |
| | (p.Asp78Gly) | | | |
| | NM_000891.2(KCNJ2): c.574A > G | 3759 | KCNJ2 | [] |
| | (p.Thr192Ala) | | | |
| 4547 | NM_000891.2(KCNJ2): c.913A > G | 3759 | KCNJ2 | [′CATACTGGAAGGCATGGTGGAAGCCVCTG |
| | (p.Thr305Ala) | | | CCATGACGACACAGTGCCGTAG′] |
| | NM_172201.1(KCNE2): c.269A > G | 9992 | KCNE2 | [] |
| | (p.Glu90Gly) | | | |
| 4548 | NM_172201.1(KCNE2): c.281A > G | 9992 | KCNE2 | [′CAGTACATTGTAGAGGACTGGCAGGRAAA |
| | (p.Glu94Gly) | | | GTACAAGAGCCAAATCTTGAAT′] |
| | NM_000335.4(SCN5A): c.1217A > G | 6331 | SCN5A | [] |
| | (p.Asn406Ser) | | | |
| | NM_000335.4(SCN5A): c.1502A > G | 6331 | SCN5A | [] |
| | (p.Asp501Gly) | | | |
| | NM_000335.4(SCN5A): c.2249A > G | 6331 | SCN5A | [] |
| | (p.Gln750Arg) | | | |
| | NM_198056.2(SCN5A): c.2527A > G | 6331 | SCN5A | [] |
| | (p.Thr843Ala) | | | |
| | NM_000335.4(SCN5A): c.2780A > G | 6331 | SCN5A | [] |
| | (p.Asn927Ser) | | | |
| | NM_000335.4(SCN5A): c.3164A > G | 6331 | SCN5A | [] |
| | (p.Asp1055Gly) | | | |
| | NM_000335.4(SCN5A): c.343A > G | 6331 | SCN5A | [] |
| | (p.Ser115Gly) | | | |
| | NM_000335.4(SCN5A): c.3755A > G | 6331 | SCN5A | [] |
| | (p.Glu1252Gly) | | | |
| 4549 | NM_000335.4(SCN5A): c.376A > G | 6331 | SCN5A | [′AAAGGATATGAGTGAACCAGAATCTYCAC |
| | (p.Lys126Glu) | | | AGCCGCTCTCCGGATGGGGTGG′] |
| 4550 | NM_198056.2(SCN5A): c.4000A > G | 6331 | SCN5A | [′TGCCCTGGTGGGCGCCATCCCGTCCRTCAT |
| | (p.Ile1334Val) | | | GAACGTCCTCCTCGTCTGCCT′] |
| | NM_000335.4(SCN5A): c.4223A > G | 6331 | SCN5A | [] |
| | (p.Tyr1408Cys) | | | |
| | NM_000335.4(SCN5A): c.4252A > G | 6331 | SCN5A | [] |
| | (p.Lys1418Glu) | | | |
| | NM_000335.4(SCN5A): c.4291A > G | 6331 | SCN5A | [] |
| | (p.Arg1431Gly) | | | |
| | NM_198056.2(SCN5A): c.4346A > G | 6331 | SCN5A | [] |
| | (p.Tyr1449Cys) | | | |
| | NM_000335.4(SCN5A): c.4412A > G | 6331 | SCN5A | [] |
| | (p.Asn1471Ser) | | | |
| 4551 | NM_198056.2(SCN5A): c.4478A > G | 6331 | SCN5A | [′ATCTTCATGACAGAGGAGCAGAAGARGTA |
| | (p.Lys1493Arg) | | | CTACAATGCCATGAAGAAGCTG′] |
| | NM_000335.4(SCN5A): c.4489A > G | 6331 | SCN5A | [] |
| | (p.Met1497Val) | | | |
| | NM_000335.4(SCN5A): c.4577A > G | 6331 | SCN5A | [] |
| | (p.Lys1526Arg) | | | |
| 4552 | NM_198056.2(SCN5A): c.4978A > G | 6331 | SCN5A | [′GATGTCCCTGCCTGCCCTCTTCAACRTCGG |
| | (p.Ile1660Val) | | | GCTGCTGCTCTTCCTCGTCAT′] |
| | NM_000335.4(SCN5A): c.5138A > G | 6331 | SCN5A | [] |
| | (p.Asp1713Gly) | | | |
| | NM_000335.4(SCN5A): c.5161A > G | 6331 | SCN5A | [] |
| | (p.Asn1721Asp) | | | |
| | NM_000335.4(SCN5A): c.5297A > G | 6331 | SCN5A | [] |
| | (p.Tyr1766Cys) | | | |
| | NM_198056.2(SCN5A): c.5302A > G | 6331 | SCN5A | [] |
| | (p.Ile1768Val) | | | |
| | NM_000335.4(SCN5A): c.5317A > G | 6331 | SCN5A | [] |
| | (p.Asn1773Asp) | | | |
| | NM_000335.4(SCN5A): c.5318A > G | 6331 | SCN5A | [] |
| | (p.Asn1773Ser) | | | |
| | NM_000335.4(SCN5A): c.5366A > G | 6331 | SCN5A | [] |
| | (p.Asp1789Gly) | | | |
| | NM_000335.4(SCN5A): c.5402A > G | 6331 | SCN5A | [] |
| | (p.Asp1801Gly) | | | |
| | NM_000335.4(SCN5A): c.5513A > G | 6331 | SCN5A | [] |
| | (p.Asp1838Gly) | | | |
| | NM_198056.2(SCN5A): c.5726A > G | 6331 | SCN5A | [] |
| | (p.Gln1909Arg) | | | |
| 4553 | NM_000335.4(SCN5A): c.688A > G | 6331 | SCN5A | [′CCGAGTCCTCCGGGCCCTGAAAACTRTATC |
| | (p.Ile230Val) | | | AGTCATTTCAGGTGAAAATCA′] |
| | NM_000335.4(SCN5A): c.715A > G | 6331 | SCN5A | [] |
| | (p.Ile239Val) | | | |
| | NM_000335.4(SCN5A): c.89A > G | 6331 | SCN5A | [] |
| | (p.Glu30Gly) | | | |
| 4554 | NM_000383.3(AIRE): c.254A > G | 326 | AIRE | [′TTCTGGAGGGTGCTGTTCAAGGACTRCAAC |
| | (p.Tyr85Cys) | | | CTGGAGCGCTATGGCCGGCTG′] |
| 4555 | NM_006920.4(SCN1A): c.1876A > G | 6323 | SCN1A | [′ACGCAACAGCAACCTGAGTCAGACCRGTA |
| | (p.Ser626Gly) | | | GGTCATCCCGGATGCTGGCAGT′] |
| 4556 | NM_006920.4(SCN1A): c.4352A > G | −1 | — | [′GAAGAAAGTCTGTACATGTATCTTTRCTTT |
| | (p.Tyr1451Cys) | | | GTTATTTTCATCATCTTTGGG′] |
| 4557 | NM_000165.4(GJA1): c.617A > G | 2697 | GJA1 | [′TGTTTCCTCTCTCGCCCCACGGAGARAACC |
| | (p.Lys206Arg) | | | ATCTTCATCATCTTCATGCTG′] |
| 4558 | NM_000833.4(GRIN2A): | 2903 | GRIN2A | [′AGCTGTGTCTTTGTGTTTGTGCTGCRGGTG |
| | c.1123 − 2A > G | | | GGCAAGTGGGAGAACCATACG′] |
| 4559 | NC_000007.14: g.62535490A > G | −1 | — | [′GGAGAGTTTTGAGGCCTGTGGTTGARATG |
| | | | | GAAATATCTTCACAGAAAAACT′] |
| 4560 | NM_004595.4(SMS): c.983A > G | 6611 | SMS | [′AATCTGACAGAAGCACTGTCGCTCTVTGA |
| | (p.Tyr328Cys) | | | AGAACAGCTGGGGCGCCTGTAT′] |
| 4561 | NM_001256864.1(DNAJC6): | 9829 | DNAJC6 | [′CAAGAGAGTGCCAACCTTCTGTTTCRGATA |
| | c.801 − 2A > G | | | CCTGGGCTATATGTGTGACCT′] |
| 4562 | NM_001204316.1(PRLR): | 5618 | PRLR | [′GTCCAGGTTCGCTGCAAACCAGACCRTGG |
| | c.635A > G (p.His212Arg) | | | ATACTGGAGTGCATGGAGTCCA′] |
| 4563 | NM_012243.2(SLC35A3): | 23443 | SLC35A3 | [′TTGGCTTCAAGATTTTGTGCCAACCRGGTA |
| | c.886A > G (p.Ser296Gly) | | | AAATGTTCTTTTCTATTTTTT′] |
| 4564 | NM_000179.2(MSH6): | 2956 | MSH6 | [′AAAGACCTTTTCCTCCCTCATTCACRGGCT |
| | c.3439 − 2A > G | | | GGCTTATTAGCTGTAATGGCC′] |
| 4565 | NM_000249.3(MLH1): c.113A > G | 4292 | MLH1 | [′AATGCTATCAAAGAGATGATTGAGARCTG |
| | (p.Asn38Ser) | | | GTACGGAGGGAGTCGAGCCGGG′] |
| 4566 | NM_000249.3(MLH1): c.122A > G | 4292 | MLH1 | [′TTTTCTGTTTGATTTGCCAGTTTAGRTGCA |
| | (p.Asp41Gly) | | | AAATCCACAAGTATTCAAGTG′] |
| 4567 | NM_000249.3(MLH1): | 4292 | MLH1 | [′TTTTTGGTTTTATTTTTTGTTTTGCNGTTCT |
| | c.1559 − 2A > G | | | CCGGGAGATGTTGCATAACC′] |
| 4568 | NM_000249.3(MLH1): | 4292 | MLH1 | [′TTGAGGTATTGAATTTCTTTGGACCRGGTG |
| | c.1990 − 2A > G | | | AATTGGGACGAAGAAAAGGAA′] |
| 4569 | NM_000249.3(MLH1): | 4292 | MLH1 | [′TTTACTCATCTTTTTGGTATCTAACVGAAA |
| | c.208 − 2A > G | | | GAAGATCTGGATATTGTATGT′] |
| 4570 | NM_000249.3(MLH1): c.544A > G | 4292 | MLH1 | [′TGGGAAAATTTTGGAAGTTGTTGGCRGGT |
| | (p.Arg182Gly) | | | ACAGTCCAAAATCTGGGAGTGG′] |
| 4571 | NM_000249.3(MLH1): | 4292 | MLH1 | [′AAAATTTTGGAAGTTGTTGGCAGGTRCAGT |
| | c.545 + 3A > G | | | CCAAAATCTGGGAGTGGGTCT′] |
| 4572 | NM_000249.3(MLH1): | 4292 | MLH1 | [′TCTTACTCTTTTGTTTTTCTTTTCCVGGTAT |
| | c.546 − 2A > G | | | TCAGTACACAATGCAGGCAT′] |
| 4573 | NM_000249.3(MLH1): | 4292 | MLH1 | [′TTGTGTCTTCTGCTGTTTGTTTATCRGCAAG |
| | c.589 − 2A > G | | | GAGAGACAGTAGCTGATGTT′] |
| 4574 | NM_000249.3(MLH1): | 4292 | MLH1 | [′GATTTTTTTATATAGGTTATCGACABACCG |
| | c.677 + 3A > G | | | ACTAACAGCATTTCCAAAGAT′] |
| 4575 | NM_000249.3(MLH1): | 4292 | MLH1 | [′ACTGGTTGCTTTCTTTTTATTGTTTRGATCG |
| | c.791 − 2A > G | | | TCTGGTAGAATCAACTTCCT′] |
| 4576 | NM_000249.3(MLH1): c.883A > G | 4292 | MLH1 | [′AAACACACACCCATTCCTGTACCTCVGGTA |
| | (p.Ser295Gly) | | | ATGTAGCACCAAACTCCTCAA′] |
| 4577 | NM_000249.3(MLH1): | 4292 | MLH1 | [′CACACCCATTCCTGTACCTCAGGTARTGTA |
| | c.884 + 4A > G | | | GCACCAAACTCCTCAACCAAG′] |
| 4578 | NM_000251.2(MSH2): | 4436 | MSH2 | [′TTTGTTTGTTTTACTACTTTCTTTTVGGAAA |
| | 2c.1277 − A > G | | | ACACCAGAAATTATTGTTGG′] |
| 4579 | NM_000251.2(MSH2): | 4436 | MSH2 | [′CTTTTTCTTTTCTTCTTGATTATCARGGCTT |
| | c.1511 − 2A > G | | | GGACCCTGGCAAACAGATTA′] |
| 4580 | NM_000251.2(MSH2): c.1660A > G | 4436 | MSH2 | [′GAAGAATGGTGTTAAATTTACCAACNGGT |
| | (p.Ser554Gly) | | | TTGCAAGTCGTTATTATATTTT′] |
| 4581 | NM_000535.5(PMS2): c.1A > G | 5395 | PMS2 | [′CCGAGGCGGATCGGGTGTTGCATCCDTGG |
| | (p.Met1Val) | | | AGCGAGCTGAGAGCTCGAGGTG′] |
| 4582 | NM_000535.5(PMS2): | 5395 | PMS2 | [′ATAAATATGTTTTCTTTTTTGCCTTRGAATG |
| | c.989 − 2A > G | | | CGTTGATATCAATGTTACTC′] |
| 4583 | NM_000059.3(BRCA2): | 675 | BRCA2 | [′TAAGGGATTTGCTTTGTTTTATTTTRGTCCT |
| | c.426 − 2A > G | | | GTTGTTCTACAATGTACACA′] |
| 4584 | NM_024876.3(ADCK4): c.857A > G | 79934 | ADCK4 | [′CAGCAGGAGCTGGCTTGGGAGTGTGRCTA |
| | (p.Asp286Gly) | | | CCGTCGTGAGGCGGCTTGTGCC′] |
| 4585 | NM_006005.3(WFS1): c.1385A > G | 7466 | WFS1 | [′TACACGCGCAGGGCCCTGGCCACCGRGGT |
| | (p.Glu462Gly) | | | CACCGCCGGCCTGCTATCGCTG′] |
| 4586 | NM_000019.3(ACAT1): c.472A > G | 38 | ACAT1 | [′GGCAGGTGGGATGGAGAGCATGTCCRATG |
| | (p.Asn158Asp) | | | TTCCATATGTAATGAACAGAGG′] |
| 4587 | NM_000050.4(ASS1): | 445 | ASS1 | [′TCCACCTGTGCTGTCTCTTTCCTGCRGCAA |
| | c.496 − 2A > G | | | CACGGGATTCCCATCCCGGTC′] |
| 4588 | NM_000117.2(EMD): | 2010 | EMD | [′CCCACTTGCTCCCCTCTTTTGCCTCRGGGA |
| | c.450 − 2A > G | | | ACGCCCCATGTACGGCCGGGA′] |
| 4589 | NM_000153.3(GALC): c.334A > G | 2581 | GALC | [′GCATAATGCATGTGGGAGGGCTCAGBGCC |
| | (p.Thr112Ala) | | | GTCTGAATAGAGGAGAGCAAAA′] |
| 4590 | NM_000155.3(GALT): | 2592 | GALT | [′CCCCACTGTCTCTCTTCTTTCTGTCRGGGG |
| | c.905 − 2A > G | | | CTCCCACAGGATCAGAGGCTG′] |
| 4591 | NM_000159.3(GCDH): c.542A > G | 2639 | GCDH | [′CTCCTGGGCTGCTTCGGGCTCACAGRGCCC |
| | (p.Glu181Gly) | | | AACAGCGGAAGTGACCCCAGC′] |
| 4592 | NM_000169.2(GLA): c.509A > G | −1 | — | [′TGGGGAGTAGATCTGCTAAAATTTGRTGGT |
| | (p.Asp170Gly) | | | TGTTACTGTGACAGTTTGGAA′] |
| 4593 | NM_000169.2(GLA): | −1 | — | [′TATTTTACCCATTGTTTTCTCATACRGGTTA |
| | c.548 − 2A > G | | | TAAGCACATGTCCTTGGCCC′] |
| 4594 | NM_000169.2(GLA): c.647A > G | −1 | — | [′TCATATCTGTTTTCACAGCCCAATTRTACA |
| | (p.Tyr216Cys) | | | GAAATCCGACAGTACTGCAAT′] |
| 4595 | NM_000232.4(SGCB): c.1A > G | 6443 | SGCB | [′GCTCGGCGGCGGCGGGCGCGGGAAGRTGG |
| | (p.Met1Val) | | | CGGCAGCGGCGGCGGCGGCTGC′] |
| 4596 | NM_000255.3(MUT): | 4594 | MUT | [′TAAAATTTTTCTTTGGGAAATTACCRGGTT |
| | c.1445 − 2A > G | | | CTGAAGTAATTGTTGGAGTAA′] |
| 4597 | NM_000277.1(PAH): c.533A > G | 5053 | PAH | [′AGTGGGCAGCCCATCCCTCGAGTGGDATA |
| | (p.Glu178Gly) | | | CATGGAGGAAGAAAAGAAAACA′] |
| 4598 | NM_000277.1(PAH): c.974A > G | 5053 | PAH | [′CAGATTGACTTTCCATTCCAGATTTRCTGG |
| | (p.Tyr325Cys) | | | TTTACTGTGGAGTTTGGGCTC′] |
| 4599 | NM_000328.2(RPGR): | 6103 | RPGR | [′TGATTATTTCTTTTTCCCTCCTACCRGGAA |
| | c.155 − 2A > G | | | ATAATAAACTTTACATGTTTG′] |
| 4600 | NM_000350.2(ABCA4): | 24 | ABCA4 | [′ATTAACGTGGGTGTCTCATTGCCTCRGAGA |
| | c.4540 − 2A > G | | | ACACAGCGCAGCACGGAAATT′] |
| 4601 | NM_000350.2(ABCA4): | 24 | ABCA4 | [′TTCTCTCTTTTTGTTTTGTTTTTCCRGATTC |
| | c.67 − 2A > G | | | GCTTTGTGGTGGAACTCGTG′] |
| 4602 | NM_000487.5(ARSA): | 410 | ARSA | [′CCCCGTGACCCCTGACTCTGCCCCCRGAGC |
| | 2c.1108 − A > G | | | CCTCGGCAGTCTCTCTTCTTC′] |
| 4603 | NM_000489.4(ATRX): | 546 | ATRX | [′TAATGTTCTTTATTTCTTCTTTTTTRGATAA |
| | c.134 − 2A > G | | | AATCAGTGGTTCTGGAAGTA′] |
| 4604 | NM_000489.4(ATRX): c.536A > G | 546 | ATRX | [′TGCACTGCTTGTGGACAACAGGTCARTCAT |
| | (p.Asn179Ser) | | | TTTCAAAAAGATTCCATTTAT′] |
| 4605 | NM_000512.4(GALNS): | 2588 | GALNS | [′CTTCTATTACCGTGGCGACACGCTGRTGGC |
| | c.1171A > G (p.Met391Val) | | | GGCCACCCTCGGGCAGCACAA′] |
| 4606 | NM_000521.3(HEXB): | 3074 | HEXB | [′ATGTATTGCAATTTGTAACGTTAATRGCTT |
| | c.1243 − 2A > G | | | GCGCCGGGCACAATAGTTGAA′] |
| 4607 | NM_000531.5(OTC): c.238A > G | 5009 | OTC | [′ATAGTATTTGCCTTTATTGCAAGGGRAGTC |
| | (p.Lys80Glu) | | | CTTAGGCATGATTTTTGAGAA′] |
| 4608 | NM_000551.3(VHL): c.233A > G | 7428 | VHL | [′GAGCCCTCCCAGGTCATCTTCTGCARTCGC |
| | (p.Asn78Ser) | | | AGTCCGCGCGTCGTGCTGCCC′] |
| 4609 | NM_001848.2(COL6A1): | 1291 | COL6A1 | [′CCAACCTTGACCTGTTTTGTGTTCCRGGGA |
| | c.805 − 2A > G | | | GAACGAGGCAAGCCGGGGCTC′] |
| 4610 | NM_001918.3(DBT): | 1629 | DBT | [′AATGAATAACAATTTAATGCTTTTCRGGCT |
| | c.773 − 2A > G | | | TTCAAAAAGCAATGGTCAAGA′] |
| 4611 | NM_003482.3(KMT2D): | 8085 | KMT2D | [′TCAACAGTGTCCTTCATTCCCCCACRGAAC |
| | c.5645 − 2A > G | | | TGCCCAAGATGGAATCCAAGG′] |
| 4612 | NM_003494.3(DYSF): | 8291 | DYSF | [′CAGGCCCTCTCTGCTCCCTTGCTCTRGGGA |
| | c.1398 − 2A > G | | | CCGCCTGACTCACAATGACAT′] |
| 4613 | NM_004006.2(DMD): | 1756 | DMD | [′AAGAGGTCATAATAGGCTTCTTTCAVATTT |
| | c.1332 − 9A > G | | | TCAGTTTACATAGAGTTTTAA′] |
| 4614 | NM_004006.2(DMD): | 1756 | DMD | [′AGTGGGATCACATGTGCCAACAGGTRTAG |
| | c.3432 + 3A > G | | | ACAATCTCTTTCACTGTGGCTT′] |
| 4615 | NM_004006.2(DMD): | 1756 | DMD | [′AAACGTTGTTGCATTTGTCTGTTTCRGTTA |
| | c.6763 − 2A > G | | | CTGGTGGAAGAGTTGCCCCTG′] |
| 4616 | NM_004006.2(DMD): | 1756 | DMD | [′AGCTGTGAATGCTTCATTCAGGCCCRAGTA |
| | c.9224 + 61934A > G | | | AATATAGGAAGAGGTGTAGTG′] |
| 4617 | NM_004006.2(DMD): | 1756 | DMD | [′GCTGTGAATGCTTCATTCAGGCCCARGTAA |
| | c.9225 − 647A > G | | | ATATAGGAAGAGGTGTAGTGG′] |
| 4618 | NM_004006.2(DMD): | 1756 | DMD | [′TGTTGCAATTTTCTTCTTCCTTTGTRGACCT |
| | c.9650 − 2A > G | | | TTTCAAGCAAGTGGCAAGTT′] |
| 4619 | NM_015560.2(OPA1): c.983A > G | 4976 | OPA1 | [′GAGATGATGACACGTTCTCCAGTTAVGGT |
| | (p.Lys328Arg) | | | AAGAACATAGGCCGTCTCAGTG′] |
| 4620 | NM_144997.5(FLCN): | 201163 | FLCN | [′TTGCTCTGCCCCTGCCCTTCTCCCCRGTGG |
| | 2c.1433 − A > G | | | GCCCCACCATCCTGAATAAGA′] |
| 4621 | NM_144997.5(FLCN): | 201163 | FLCN | [′AGATTTACTTTTCCTTTTCATGGACRGGGC |
| | c.250 − 2A > G | | | TGCCGGTCACTTGCTGCAGGG′] |
| 4622 | NM_000146.3(FTL): c.1A > G | 2512 | FTL | [′GTTAGCTCCTTCTTGCCAACCAACCRTGAG |
| | (p.Met1Val) | | | CTCCCAGATTCGTCAGAATTA′] |
| 4623 | NM_024531.4(SLC52A2): | 79581 | SLC52A2 | [′GTGCAGAGCTTTTCCTGCTTACCCTRCGGG |
| | c.914A > G (p.Tyr305Cys) | | | CGTCTGGCCTACCACCTGGCT′] |
| 4624 | NM_001743.5(CALM2): c.293A > G | 805 | CALM2 | [′TACTAAATTTTTTGCTAGGATGGCADTGGC |
| | (p.Asn98Ser) | | | TATATTAGTGCTGCAGAACTT′] |
| 4625 | NM_014754.2(PTDSS1): c.1058A > | 9791 | PTDSS1 | [′ACACAGTGCAAGCGCGTAGGAACACRATG |
| | G (p.Gln353Arg) | | | CTGGGTGTTTGGGTGAGTAATC′] |
| 4626 | NM_052844.3(WDR34): c.1307A > | 89891 | WDR34 | [′GGACCAGCGCACAGCAAACAGATACYTGA |
| | G (p.Lys436Arg) | | | GGGAGAGCTGCAGCGAAGTCAA′] |
| 4627 | NM_001130010.2(C15orf41): | 84529 | C15orf41 | [′TACTTCTGTTTTATAAAGGTGGACTVTGCG |
| | c.281A > G (p.Tyr94Cys) | | | CCCTCATTAATGGCTCGGCTT′] |
| 4628 | NM_001290048.1(ATL3): c.521A > | 25923 | ATL3 | [′AATTTCATCCATTGCCAGACGACCGYATTC |
| | G (p.Tyr174Cys) | | | TGTGAAGAGCTTTAAAAAAGA′] |
| 4629 | NM_000531.5(OTC): c.1034A > G | 5009 | OTC | [′GTCATGGTGTCCCTGCTGACAGATTRCTCA |
| | (p.Tyr345Cys) | | | CCTCAGCTCCAGAAGCCTAAA′] |
| 4630 | NM_000531.5(OTC): c.122A > G | 5009 | OTC | [′AATAAAGTGCAGCTGAAGGGCCGTGRCCT |
| | (p.Asp41Gly) | | | TCTCACTCTAAAAAACTTTACC′] |
| 4631 | NM_000531.5(OTC): c.155A > G | 5009 | OTC | [′ACTCTAAAAAACTTTACCGGAGAAGRAAT |
| | (p.Glu52Gly) | | | TAAATATATGCTATGGCTATCA′] |
| 4632 | NM_000531.5(OTC): c.1A > G | 5009 | OTC | [′TCGTCCTTTACACAATTAAAAGAAGDTGCT |
| | (p.Met1Val) | | | GTTTAATCTGAGGATCCTGTT′] |
| 4633 | NM_000531.5(OTC): c.268A > G | 5009 | OTC | [′CTTAGGCATGATTTTTGAGAAAAGARGTA |
| | (p.Ser90Gly) | | | CTCGAACAAGATTGTCTACAGA′] |
| 4634 | NM_000531.5(OTC): c.277A > G | 5009 | OTC | [′GATTTTTGAGAAAAGAAGTACTCGARCAA |
| | (p.Thr93Ala) | | | GATTGTCTACAGAAACAGGTAA′] |
| 4635 | NM_000531.5(OTC): c.350A > G | 5009 | OTC | [′TGTTTTCTTACCACACAAGATATTCDTTTG |
| | (p.His117Arg) | | | GGTGTGAATGAAAGTCTCACG′] |
| 4636 | NM_000531.5(OTC): c.377A > G | 5009 | OTC | [′TTGGGTGTGAATGAAAGTCTCACGGRCAC |
| | (p.Asp126Gly) | | | GGCCCGGTTTGTAAATATTTTC′] |
| 4637 | NM_000531.5(OTC): | 5009 | OTC | [′GATTATCTTTTTCTTGGTTTGCCACNGTGT |
| | c.387 − 2A > G | | | ATTGTCTAGCATGGCAGATGC′] |
| 4638 | NM_000531.5(OTC): c.481A > G | 5009 | OTC | [′TAAAGAAGCATCCATCCCAATTATCRATG |
| | (p.Asn161Asp) | | | GGCTGTCAGATTTGTACCATCC′] |
| 4639 | NM_000531.5(OTC): c.482A > G | 5009 | OTC | [′AAAGAAGCATCCATCCCAATTATCARTGG |
| | (p.Asn161Ser) | | | GCTGTCAGATTTGTACCATCCT′] |
| 4640 | NM_000531.5(OTC): c.503A > G | 5009 | OTC | [′ATCAATGGGCTGTCAGATTTGTACCVTCCT |
| | (p.His168Arg) | | | ATCCAGATCCTGGCTGATTAC′] |
| 4641 | NM_000531.5(OTC): c.524A > G | 5009 | OTC | [′TACCATCCTATCCAGATCCTGGCTGDTTAC |
| | (p.Asp175Gly) | | | CTCACGCTCCAGGTTGGTTTA′] |
| 4642 | NM_000531.5(OTC): c.527A > G | 5009 | OTC | [′CATCCTATCCAGATCCTGGCTGATTRCCTC |
| | (p.Tyr176Cys) | | | ACGCTCCAGGTTGGTTTATTT′] |
| 4643 | NM_000531.5(OTC): | 5009 | OTC | [′TCATCTCCTTCATCCCGTGCCTTTTRGGAA |
| | c.541 − 2A > G | | | CACTATAGCTCTCTGAAAGGT′] |
| 4644 | NM_000531.5(OTC): c.542A > G | 5009 | OTC | [′TCTCCTTCATCCCGTGCCTTTTAGGRACAC |
| | (p.Glu181Gly) | | | TATAGCTCTCTGAAAGGTCTT′] |
| 4645 | NM_000531.5(OTC): c.548A > G | 5009 | OTC | [′TCATCCCGTGCCTTTTAGGAACACTRTAGC |
| | (p.Tyr183Cys) | | | TCTCTGAAAGGTCTTACCCTC′] |
| 4646 | NM_000531.5(OTC): c.595A > G | 5009 | OTC | [′CCTCAGCTGGATCGGGGATGGGAACRATA |
| | (p.Asn199Asp) | | | TCCTGCACTCCATCATGATGAG′] |
| 4647 | NM_000531.5(OTC): c.596A > G | 5009 | OTC | [′CTCAGCTGGATCGGGGATGGGAACARTAT |
| | (p.Asn199Ser) | | | CCTGCACTCCATCATGATGAGC′] |
| 4648 | NM_000531.5(OTC): c.613A > G | 5009 | OTC | [′TGGGAACAATATCCTGCACTCCATCRTGAT |
| | (p.Met205Val) | | | GAGCGCAGCGAAATTCGGAAT′] |
| 4649 | NM_000531.5(OTC): c.716A > G | 5009 | OTC | [′AAGTTGGCAGAGCAGTATGCCAAAGDGGT |
| | (p.Glu239Gly) | | | ATGCTCTTTACATGTAAAGCTA′] |
| 4650 | NM_000531.5(OTC): | 5009 | OTC | [′TGGCAGAGCAGTATGCCAAAGAGGTRTGC |
| | c.717 + 3A > G | | | TCTTTACATGTAAAGCTATTAT′] |
| 4651 | NM_000531.5(OTC): | 5009 | OTC | [′TTTAACCAGCGTGTTTATGTATGCTRGAAT |
| | c.718 − 2A > G | | | GGTACCAAGCTGTTGCTGACA′] |
| 4652 | NM_000531.5(OTC): c.788A > G | 5009 | OTC | [′CATGGAGGCAATGTATTAATTACAGRCAC |
| | (p.Asp263Gly) | | | TTGGATAAGCATGGGACAAGAA′] |
| 4653 | NM_000531.5(OTC): c.790A > G | 5009 | OTC | [′TGGAGGCAATGTATTAATTACAGACRCTTG |
| | (p.Thr264Ala) | | | GATAAGCATGGGACAAGAAGA′] |
| 4654 | NM_000531.5(OTC): c.905A > G | 5009 | OTC | [′GCTGCCTCTGACTGGACATTTTTACDCTGC |
| | (p.His302Arg) | | | TTGCCCAGAAAGCCAGAAGAA′] |
| 4655 | NM_000531.5(OTC): c.929A > G | 5009 | OTC | [′CACTGCTTGCCCAGAAAGCCAGAAGRAGT |
| | (p.Glu310Gly) | | | GGATGATGAAGTCTTTTATTCT′] |
| 4656 | NM_000531.5(OTC): c.988A > G | 5009 | OTC | [′ACTAGTGTTCCCAGAGGCAGAAAACRGAA |
| | (p.Arg330Gly) | | | AGTGGACAATCATGGTAAGCAA′] |
| 4657 | NM_000322.4(PRPH2): c.422A > G | 5961 | PRPH2 | [′GGGCTCAAGAACGGCATGAAGTACTRCCG |
| | (p.Tyr141Cys) | | | GGACACAGACACCCCTGGCAGG′] |
| | NM_003590.4(CUL3): | 8452 | CUL3 | [] |
| | c.1207 − 26A > G | | | |
| | NM_003590.4(CUL3): c.1376A > G | 8452 | CUL3 | [] |
| | (p.Lys459Arg) | | | |
| | NM_003590.4(CUL3): | 8452 | CUL3 | [] |
| | c.1377 + 3A > G | | | |
| 4658 | NM_017415.2(KLHL3): c.232A > G | 26249 | KLHL3 | [′AGCCTGCAGCCCCTACTTCTGTGCGDTGTT |
| | (p.Met78Val) | | | CACAGGTATGGTGAGTGGCCA′] |
| | NM_017415.2(KLHL3): c.926A > G | 26249 | KLHL3 | [] |
| | (p.Gln309Arg) | | | |
| 4659 | NM_005763.3(AASS): c.874A > G | 10157 | AASS | [′CTCACATCAGTATTAAAACGACTTAYGTA |
| | (p.Ile292Val) | | | GCGCTCCGGATGTTTGTCATAC′] |
| 4660 | NM_000203.4(IDUA): c.1874A > G | 3425 | IDUA | [′TCCTACCGAGTTCGAGCCCTGGACTRCTGG |
| | (p.Tyr625Cys) | | | GCCCGACCAGGCCCCTTCTCG′] |
| 4661 | NM_000546.5(TP53): c.736A > G | 7157 | TP53 | [′GTGTAACAGTTCCTGCATGGGCGGCVTGA |
| | (p.Met246Val) | | | ACCGGAGGCCCATCCTCACCAT′] |
| 4662 | NM_194442.2(LBR): c.1639A > G | 3930 | LBR | [′GCCATGATGAGATCACCCAAGTAATYGGG |
| | (p.Asn547Asp) | | | GTGGCGAACAAAGCCCCACCAT′] |
| 4663 | NM_000090.3(COL3A1): | 1281 | COL3A1 | [′ATTAAAAAATATTTTTATTTCCTCTRGGGT |
| | c.2284 − 2A > G | | | CCTACTGGTCCTATTGGTCCT′] |
| | (p.Gly762_Lys779del) | | | |
| 4664 | NM_000090.3(COL3A1): | 1281 | COL3A1 | [′TCTTGAAATTGTATTTAATTTTTTCRGGGC |
| | c.997 − 2A > G | | | CCTCCTGGTCCTCCTGGAACT′] |
| | (p.Gly333_Lys350del+) | | | |
| 4665 | NM_000090.3(COL3A1): | 1281 | COL3A1 | [′CATTGTGATGTCATGATACTTTCTTDGGGA |
| | c.3418 − 2A > G | | | CCTGTTGGACCCAGTGGACCT′] |
| | (p.Arg1139_Gly1140ins | | | |
| | VSSTERYYRSTCFRCLHFRKIFWHCDVMILSW) | | | |
| 4666 | NM_000090.3(COL3A1): | 1281 | COL3A1 | [′AAAAGAGCTCTTGAAATTGTATTTARTTTT |
| | c.997 − 10A > G | | | TTCAGGGCCCTCCTGGTCCTC′] |
| | (p.Pro332_Gly333insFFQ) | | | |
| 4667 | NM_000090.3(COL3A1): | 1281 | COL3A1 | [′ACAGAGTGTATCATTATACTTTTCTDGGGG |
| | c.1870 − 2A > G | | | CCTGGTGGTGACAAAGGAGAC′] |
| | (p.Gly624_Gln641del) | | | |
| 4668 | NM_000090.3(COL3A1): | 1281 | COL3A1 | [′CTTCTCAATTGAATGTTTTCATCTTRGGGC |
| | 2c.3202 − A > G | | | CCTGCTGGCCCTGCTGGTGCT′] |
| | (p.Gly1068_Pro1085del) | | | |
| 4669 | NM_000090.3(COL3A1): | 1281 | COL3A1 | [′AGTAAATACCGACCACTTCTTCTTTRGGGT |
| | 2c.1762 − A > G | | | GCTCCTGGTAAGAATGGAGAA′] |
| | (p.Gly588_Gln605del) | | | |
| 4670 | NM_003611.2(OFD1): | 8481 | OFD1 | [′CTTTTTGGCAATAATGAAAAGGTAARTTGA |
| | c.935 + 706A > G | | | TCAAGAGAGAGGAAATAGGCA′] |
| 4671 | NM_014740.3(EIF4A3): c.809A > G | 9775 | EIF4A3 | [′AGTGATGGTCAGTGTGTCGTAGAGGYCAC |
| | (p.Asp270Gly) | | | ACAGAGTGTCAAATTTCCACTC′] |
| 4672 | NM_001040436.2(YARS2): | 51067 | YARS2 | [′TTTGTTACTTGTTGGTGATTTATGCYGACT |
| | c.1303A > G (p.Ser435Gly) | | | CCGCCTTCTGTTATCATTCGA′] |
| 4673 | NM_000277.1(PAH): c.1157A > G | 5053 | PAH | [′ACTGTCACGGAGTTCCAGCCCCTCTRTTAC |
| | (p.Tyr386Cys) | | | GTGGCAGAGAGTTTTAATGAT′] |
| 4674 | NM_000277.1(PAH): c.812A > G | 5053 | PAH | [′TTCCACTGCACACAGTACATCAGACDTGG |
| | (p.His271Arg) | | | ATCCAAGCCCATGTATACCCCC′] |
| 4675 | NM_004820.3(CYP7B1): c.889A > G | 9420 | CYP7B1 | [′ATTGCCCAGAACATAGTTGGAATAGYGTT |
| | (p.Thr297Ala) | | | TGCCACAGAGGCCCAGAGAAAG′] |
| 4676 | NM_024301.4(FKRP): c.1A > G | 79147 | FKRP | [′CCAGCTAGCCCCAGACTTCGGCCCCRTGCG |
| | (p.Met1Val) | | | GCTCACCCGCTGCCAGGCTGC′] |
| 4677 | NM_001955.4(EDN1): c.271A > G | 1906 | EDN1 | [′GTATGGACTTGGAAGCCCTAGGTCCRAGA |
| | (p.Lys91Glu) | | | GAGCCTTGGAGAATTTACTTCC′] |
| 4678 | NM_198947.3(FAM111B): | 374393 | FAM111B | [′TAATGTATACTGTATGTTTACCCAARGAAG |
| | c.1879A > G (p.Arg627Gly) | | | TTTCCTATCAGAGGTTTGGAA′] |
| 4679 | NM_001282227.1(CECR1): | 51816 | CECR1 | [′GCCCATGAAGACCTCATAGAAATCAYAGG |
| | c.1232A > G (p.Tyr411Cys) | | | ACAAGCCTTTGGCACCAAACAT′] |
| 4680 | NM_033419.4(PGAP3): c.914A > G | 93210 | PGAP3 | [′TGATTCCTTCAGCAGGTACAGGCTGYCATC |
| | (p.Asp305Gly) | | | TTCCAGAAAGCTGTGGGCCAA′] |
| 4681 | NM_007294.3(BRCA1): | 672 | BRCA1 | [′TGTTCTTTCTTTCTTTATAATTTATRGATTT |
| | c.135 − 2A > G | | | TGCATGCTGAAACTTCTCAA′] |
| 4682 | NM_007294.3(BRCA1): | 672 | BRCA1 | [′AAATTAAACTTCTCCCATTCCTTTCRGAGG |
| | c.4676 − 2A > G | | | GAACCCCTTACCTGGAATCTG′] |
| 4683 | NM_015474.3(SAMHD1): | 25939 | SAMHD1 | [′GGAAATGACAATCAAGTTTCTTACAYTGT |
| | c.1153A > G (p.Met385Val) | | | ATCAATAATGTTGCCAACTTTG′] |
| 4684 | NM_015474.3(SAMHD1): | 25939 | SAMHD1 | [′TTTTGGAAGAGATTCATAGTCCTCCYGGAA |
| | c.1411 − 2A > G | | | AACACAAGACTCCCCATGTTA′] |
| 4685 | NM_000097.5(CPOX): c.980A > G | 1371 | CPOX | [′ACCAATGCCCCGCCGTTCTCCACGAYGGG |
| | (p.His327Arg) | | | CTATAAAGAAGTAATCATCACA′] |
| 4686 | NM_012338.3(TSPAN12): c.413A | 23554 | TSPAN12 | [′AAGCCACCGATATCTAGGTAATCCAYAAT |
| | G (p.Tyr138Cys) | | | TTGTCATCCTGGCTTTCAAAGT′] |
| 4687 | NM_000406.2(GNRHR): c.94A > G | 2798 | GNRHR | [′GTCACTCGGATCTTTCCAGACAAGGYCAG |
| | (p.Thr32Ala) | | | AGTGGGGAGGTTGCCCTGCATC′] |
| 4688 | NM_199189.2(MATR3): c.1864A > | 9782 | MATR3 | [′TGGTTCCCAGAAGACTGAGAGTTCARCCG |
| | G (p.Thr622Ala) | | | AAGGTAAAGAACAAGAAGAGAA′] |
| 4689 | NM_003181.3(T): c.512A > G | 6862 | T | [′CTGTGGACCCCCAACTCTCACTATGYGGAT |
| | (p.His171Arg) | | | TCGAGGCTCATACTTATGCAA′] |
| 4690 | NM_000321.2(RB1): c.1927A > G | 5925 | RB1 | [′AGCCTTCCAGACCCAGAAGCCATTGRAAT |
| | (p.Lys643Glu) | | | CTACCTCTCTTTCACTGTTTTA′] |
| 4691 | NM_002234.3(KCNA5): c.143A > G | 3741 | KCNA5 | [′GCTGGGCTCAGCGATGGGCCCAAGGRGCC |
| | (p.Glu48Gly) | | | GGCGCCAAAGGGGCGCGGCGCG′] |
| 4692 | NM_178014.3(TUBB): c.895A > G | 203068 | TUBB | [′CCAGCAGGTCTTCGATGCCAAGAACRTGA |
| | (p.Met299Val) | | | TGGCTGCCTGTGACCCCCGCCA′] |
| 4693 | NM_000136.2(FANCC): | 2176 | FANCC | [′TGCTTCTATTTGTTCCCTTTCTTACRGATTT |
| | c.−78 − 2A > G | | | AATGTGTGCCGACCATTTCC′] |
| 4694 | NM_000546.5(TP53): c.488A > G | 7157 | TP53 | [′CTCCGTCATGTGCTGTGACTGCTTGYAGAT |
| | (p.Tyr163Cys) | | | GGCCATGGCGCGGACGCGGGT′] |
| 4695 | NM_000546.5(TP53): c.659A > G | 7157 | TP53 | [′TTTCGACATAGTGTGGTGGTGCCCTVTGAG |
| | (p.Tyr220Cys) | | | CCGCCTGAGGTCTGGTTTGCA′] |
| 4696 | NM_000546.5(TP53): c.701A > G | 7157 | TP53 | [′GGCTCTGACTGTACCACCATCCACTRCAAC |
| | (p.Tyr234Cys) | | | TACATGTGTAACAGTTCCTGC′] |
| 4697 | NM_002878.3(RAD51D): c.1A > G | −1 | — | [′CACAGTCCGACCCTGAGCACGCCCAYGTT |
| | (p.Met1Val) | | | CCCCGCAGGCCGGAACAGCCCC′] |
| 4698 | NM_058216.2(RAD51C): | 5889 | RAD51C | [′ATCTAATATTATCTCTTCTGTATTTRGGTTC |
| | c.706 − 2A > G | | | GACTAGTGATAGTGGATGGT′] |
| 4699 | NM_015713.4(RRM2B): c.556A > G | 50484 | RRM2B | [′TCTACAGCAGCAAAGGCCACCACTCYTTC |
| | (p.Arg186Gly) | | | CCCTGGGAGACATAAAATCGTT′] |
| 4700 | NM_015713.4(RRM2B): c.581A > G | 50484 | RRM2B | [′AAAAGATCCTGAGAAGAAAACTCCTYCTA |
| | (p.Glu194Gly) | | | CAGCAGCAAAGGCCACCACTCT′] |
| 4701 | NM_000219.5(KCNE1): c.242A > G | 3753 | KCNE1 | [′CACTCGAACGACCCATTCAACGTCTDCATC |
| | (p.Tyr81Cys) | | | GAGTCCGATGCCTGGCAAGAG′] |
| 4702 | NM_000271.4(NPC1): c.1832A > G | 4864 | NPC1 | [′TTCACTGCTGAACGAAGTATTGAAGRTGA |
| | (p.Asp611Gly) | | | ACTAAATCGTGAAAGTGACAGT′] |
| 4703 | NM_000257.3(MYH7): c.1952A > G | 4625 | MYH7 | [′CTGGGGCTGTGTCCCACTCACCCTGYGCAG |
| | (p.His651Arg) | | | AGCTGACACAGTCTGAAAGGA′] |
| 4704 | NM_000540.2(RYR1): c.7043A > G | 6261 | RYR1 | [′GGGTGGCCAGGCGAGAGCGTGGAGGRGA |
| | (p.Glu2348Gly) | | | ACGCCAATGTGGTGGTGCGGCTG′] |
| 4705 | NM_058216.2(RAD51C): | 5889 | RAD51C | [′TTTGTATATATATTTTTTATCTTTCRGCCTC |
| | c.1027 − 2A > G | | | AGGGATTTAGAGATACTGTT′] |
| 4706 | NM_130838.1(UBE3A): | 7337 | UBE3A | [′TGTATTTTTAAAAATCATTTCTTATRGGTA |
| | c.1694 − 2A > G | | | TGTTCACATACGATGAATCTA′] |
| 4707 | NM_022168.3(IFIH1): c.1009A > G | 64135 | IFIH1 | [′TCCTTGGCAATGTAAACAGCCACTCYGGTT |
| | (p.Arg337Gly) | | | TTTCCACTCCCTGTAGGGAGG′] |
| 4708 | NM_022068.3(PIEZO2): c.2134A > | 63895 | PIEZO2 | [′GCCACACAGAACAGGAACAGCACCAYGTA |
| | G (p.Met712Val) | | | GATGATTTTGTACATTACGATT′] |
| 4709 | NM_001164342.2(ZBTB20): | 26137 | ZBTB20 | [′ACCGCCAAACAGAACTACGTCAAGCRCAT |
| | c.1787A > G (p.His596Arg) | | | GTTCGTACACACAGGTGAGTGT′] |
| 4710 | NM_001128159.2(VPS53): | 55275 | VPS53 | [′TGCCTGATACTACGTCCATCTCACCYGTTC |
| | c.2084A > G (p.Gln695Arg) | | | TGCTCCCACCATGCTAATTGG′] |
| 4711 | NM_003108.3(SOX11): c.347A > G | 6664 | SOX11 | [′AAGCACATGGCCGACTACCCCGACTRCAA |
| | (p.Tyr116Cys) | | | GTACCGGCCCCGGAAAAAGCCC′] |
| 4712 | NM_020435.3(GJC2): | 57165 | GJC2 | [′TTCAGACCCTGAGGCCGAGGGGGGARCAA |
| | c.−170A > G | | | TGGGGCCCTTGAGGGCCCCTCC′] |
| 4713 | NM_004817.3(TJP2): | 9414 | TJP2 | [′GATTTACTTCCCGTGGTTTCTTCTCRGAGC |
| | c.1992 − 2A > G | | | TGAACAAATGGCCAGTGTTCA′] |
| 4714 | NM_017565.3(FAM20A): | −1 | — | [′GGTGTCTCCTCATCTCGCTGCTGTCYGGAA |
| | c.720 − 2A > G | | | GGAAGGAAGGAATCACGCCCT′] |
| 4715 | NM_002764.3(PRPS1): c.343A > G | 5631 | PRPS1 | [′AATCTCAGCCAAGCTTGTTGCAAATRTGCT |
| | (p.Met115Val) | | | ATCTGTAGCAGGTGCAGATCA′] |
| 4716 | NM_000216.2(ANOS1): c.1A > G | 3730 | ANOS1 | [′TCGCCCTCGCCCTCGACCCGCAGCCRTGGT |
| | (p.Met1Val) | | | GCCCGGGGTGCCCGGCGCGGT′] |
| 4717 | NM_031229.2(RBCK1): c.1160A > | 10616 | RBCK1 | [′TGGTGCTTCTTTGAGGATGATGTCARTGAG |
| | G (p.Asn387Ser) | | | TTCACCTGCCCTGTGTGTTTC′] |
| 4718 | NM_015599.2(PGM3): c.737A > G | 5238 | PGM3 | [′CACAAAGTCAGCTCCACATAAATGAYTGA |
| | (p.Asn246Ser) | | | GTTTGCCCTTGGACCCATCATT′] |
| 4719 | NM_015599.2(PGM3): c.1352A > G | 5238 | PGM3 | [′TTTCTCTCACACCTGAACTTTAAGTYGTCT |
| | (p.Gln451Arg) | | | GTTTGGAAGATCTGTATAGAG′] |
| 4720 | NM_000535.5(PMS2): | 5395 | PMS2 | [′CTTTTCTACTCCTTGTATTTTGTGCRGGTCT |
| | c.904 − 2A > G | | | GCAGACTCGTGAATGAGGTC′] |
| 4721 | NM_000546.5(TP53): c.358A > G | 7157 | TP53 | [′GGGCTTCTTGCATTCTGGGACAGCCDAGTC |
| | (p.Lys120Glu) | | | TGTGACTTGCACGGTCAGTTG′] |
| 4722 | NM_000546.5(TP53): c.842A > G | 7157 | TP53 | [′CGTGTTTGTGCCTGTCCTGGGAGAGDCCGG |
| | (p.Asp281Gly) | | | CGCACAGAGGAAGAGAATCTC′] |
| 4723 | NM_001128425.1(MUTYH): | 4595 | MUTYH | [′CCCCTGCCTGGCTGCCCTCCCTCTCRGGTC |
| | c.1187 − 2A > G | | | TGCTGGCAGGACTGTGGGAGT′] |
| 4724 | NM_000546.5(TP53): | 7157 | TP53 | [′TCTCCTCCCTGCTTCTGTCTCCTACRGCCAC |
| | c.1101 − 2A > G | | | CTGAAGTCCAAAAAGGGTCA′] |
| 4725 | NM_000314.6(PTEN): | 5728 | PTEN | [′GGCTTCTCTTTTTTTTCTGTCCACCRGGGA |
| | c.493 − 2A > G | | | GTAACTATTCCCAGTCAGAGG′] |
| 4726 | NM_000051.3(ATM): | 472 | ATM | [′ATATATTTTAATTTTGTGCCCTTGCRGATT |
| | c.3994 − 2A > G | | | GATCACTTATTCATTAGTAAT′] |
| 4727 | NM_000314.6(PTEN): c.403A > G | 5728 | PTEN | [′TGGAAAGGGACGAACTGGTGTAATGRTAT |
| | (p.Ile135Val) | | | GTGCATATTTATTACATCGGGG′] |
| 4728 | NM_000249.3(MLH1): | 4292 | MLH1 | [′TAAATTATTTTCTGTTTGATTTGCCDGTTTA |
| | c.117 − 2A > G | | | GATGCAAAATCCACAAGTAT′] |
| 4729 | NM_025150.4(TARS2): | 80222 | TARS2 | [′GGTCCAACAGCAACAGTATATGGGTRAGA |
| | c.695 + 3A > G | | | GTTGTCAAGATTAAGGCAAACA′] |
| 4730 | NM_206933.2(USH2A): c.10544A > | 7399 | USH2A | [′TGGACCAAAATAGACAATCTTGAAGVTAC |
| | G (p.Asp3515Gly) | | | AATTGTCTTAAACTGGAGAAAA′] |
| 4731 | NM_000834.3(GRIN2B): c.1238A > | 2904 | GRIN2B | [′CATCTGAGCATTGTGACCCTGGAGGRGGC |
| | G (p.Glu413Gly) | | | ACCATTTGTCATTGTGGAAAGT′] |
| 4732 | NM_004830.3(MED23): c.3638A > | 9439 | MED23 | [′TGTAGCTATACGTTAGCTCTTGCACRTGCT |
| | G (p.His1213Arg) | | | GTGTGGCACCATTCTAGCATC′] |
| 4733 | NM_003159.2(CDKL5): c.125A > G | 6792 | CDKL5 | [′GAAACACATGAAATTGTGGCGATCARGAA |
| | (p.Lys42Arg) | | | ATTCAAGGACAGTGAAGGTAGA′] |
| 4734 | NM_003159.2(CDKL5): c.380A > G | 6792 | CDKL5 | [′CTAATCAAGGCTATTCACTGGTGCCRTAAG |
| | (p.His127Arg) | | | AATGATATTGTCCATCGAGGT′] |
| 4735 | NM_003159.2(CDKL5): c.578A > G | 6792 | CDKL5 | [′AGCGCTCCCTATGGAAAGTCCGTGGRCAT |
| | (p.Asp193Gly) | | | GTGGTCGGTGGGCTGTATTCTT′] |
| 4736 | NM_198282.3(TMEM173): | 340061 | TMEM173 | [′TGACCATGCCAGCCCATGGGCCACGYTGA |
| | c.461A > G (p.Asn154Ser) | | | AATTCCCTTTTTCACACACTGC′] |
| 4737 | NM_032018.6(SPRTN): c.350A > G | 83932 | SPRTN | [′CTCCTGCATGAAATGATACATGCCTRTTTA |
| | (p.Tyr117Cys) | | | TTTGTCACTAATAACGACAAA′] |
| 4738 | NM_001759.3(CCND2): c.838A > G | 894 | CCND2 | [′GGAGGATGAACTGGACCAAGCCAGCRCCC |
| | (p.Thr280Ala) | | | CTACAGACGTGCGGGATATCGA′] |
| 4739 | NM_007315.3(STAT1): c.854A > G | 6772 | STAT1 | [′AGGGTCATGTTCGTAGGTGTATTTCYGTTC |
| | (p.Gln285Arg) | | | CAATTCCTCCAACTTTTTAAG′] |
| 4740 | NM_139276.2(STAT3): c.1175A > G | 6774 | STAT3 | [′GTTGGATTCTTCCATGTTCATCACTYTTGT |
| | (p.Lys392Arg) | | | GTTTGTGCCCAGAATGTTAAA′] |
| 4741 | NM_001037811.2(HSD17B10): | 3028 | HSD17B10 | [′GATGCCTGCACAGTTGACAGCTACAYCCA |
| | 7c.25A > G (p.Asp86Gly) | | | CACGGCCAAACTTTCCTTTTGC′] |
| 4742 | NM_005726.5(TSFM): | 10102 | TSFM | [′CGCGCGGACCGGGAGCTACCCGGTGRGAA |
| | c.57 + 4A > G | | | GTCCTGGTGCTGGTACCGACCT′] |
| 4743 | NM_000166.5(GJB1): c.580A > G | 2705 | GJB1 | [′CGAGAAAACCGTCTTCACCGTCTTCRTGCT |
| | (p.Met194Val) | | | AGCTGCCTCTGGCATCTGCAT′] |
| 4744 | NM_001077494.3(NFKB2): | 4791 | NFKB2 | [′TTACCCCCAGCAGAGGTGAAGGAAGRCAG |
| | c.2594A > G (p.Asp865Gly) | | | TGCGTACGGGAGCCAGTCAGTG′] |
| 4745 | NC_012920.1: m.5559A > G | 4578 | MT-TW | [′TGTTACAGAAATTAAGTATTGCAACYTACT |
| | | | | GAGGGCTTTGAAGGCTCTTGG′] |
| 4746 | NC_012920.1: m.13514A > G | 4540 | MT-ND5 | [′TTCCTCACAGGTTTCTACTCCAAAGRCCAC |
| | | | | ATCATCGAAACCGCAAACATA′] |
| 4747 | NM_007315.3(STAT1): c.2018A > G | 6772 | STAT1 | [′GTAATACTTTCCAAAGGCATGGTCTYTGTC |
| | (p.Lys673Arg) | | | AATATTTGGATACAGATACTT′] |
| 4748 | NM_007315.3(STAT1): c.1909A > G | 6772 | STAT1 | [′GGGAAAGTAACAGCAGAAAGTTCTTYCTT |
| | (p.Lys637Glu) | | | CGTGTAGGGTTCAACCGCATGG′] |
| 4749 | NM_000060.3(BTD): c.683A > G | 686 | BTD | [′GCATTCGATGTTCCTCTTAAAGTGGRTCTC |
| | (p.Asp228Gly) | | | ATCACCTTTGATACCCCCTTT′] |
| 4750 | NM_004992.3(MECP2): | 4204 | MECP2 | [′TACTTACATACTTGTTTAACACTTCRGGGA |
| | c.27 − 2A > G | | | AGAAAAGTCAGAAGACCAGGA′] |
| 4751 | NM_004992.3(MECP2): | 4204 | MECP2 | [′CTTGTGTCTTTCTGTTTGTCCCCACNGTCCC |
| | c.378 − 2A > G | | | CAGGGAAAAGCCTTTCGCTC′] |
| 4752 | NM_003159.2(CDKL5): | 6792 | CDKL5 | [′TTTCCTTCTGCTTCTTTTCCCTTGCRGGAAA |
| | 2c.100 − A > G | | | CACATGAAATTGTGGCGATC′] |
| 4753 | NM_003159.2(CDKL5): | 6792 | CDKL5 | [′ACAACTTTGGACTTTGCTATCTTTCRGGTT |
| | 2c.464 − A > G | | | TTGCTCGTAATCTGTCAGAAGT |
| 4754 | NM_003159.2(CDKL5): | 6792 | CDKL5 | [′GATATACTTCTTTTGTTTTTAACATRGAAA |
| | c.978 − 2A > G | | | CCAAGCCGGCAAAAGTACTGC′] |
| 4755 | NM_032492.3(JAGN1): c.485A > G | 84522 | JAGN1 | [′TTGGCAGTGCAAGTGCATGCCTGGCRGTTG |
| | (p.Gln162Arg) | | | TACTACAGCAAGAAGCTCCTA′] |
| 4756 | NM_178517.3(PIGW): c.499A > G | 284098 | PIGW | [′AACTGAGCTCTATGGGACAGGAGCARTGG |
| | (p.Met167Val) | | | ATTTTGGAGTAGGTGGCTTTGT′] |
| 4757 | NM_004387.3(NKX2-5): c.461A > G | 1482 | NKX2-5 | [′TCGCAGGCGCAGGTCTATGAGCTGGRGCG |
| | (p.Glu154Gly) | | | GCGCTTCAAGCAGCAGCGGTAC′] |
| 4758 | NM_005859.4(PURA): c.289A > G | 5813 | PURA | [′CGCCGAGGTGGGCGCGGGCGGCAACRAGA |
| | (p.Lys97Glu) | | | GCCGCCTTACTCTCTCCATGTC′] |
| 4759 | NM_004046.5(ATP5A1): c.962A > G | 498 | ATP5A1 | [′GCGGAGCAACAGAGACATCTGACGGHAAG |
| | (p.Tyr321Cys) | | | CAACAGCCTATGGTACAGAATA′] |
| 4760 | NM_000038.5(APC): | 324 | APC | [′GATGACCCATATTCTGTTTCTTACTRGGAA |
| | c.1744 − 2A > G | | | TCAACCCTCAAAAGCGTATTG′] |
| 4761 | NM_000751.2(CHRND): c.1319A > | 1144 | CHRND | [′TTCAATGAGCTGAAGCCAGCTGTGGWTGG |
| | G (p.Asp440Gly) | | | GGCAAACTTCATTGTTAACCAC′] |
| 4762 | NM_130466.3(UBE3B): c.1A > G | 89910 | UBE3B | [′GCAGGGTTTGTGCAAGTTTGCAAACRTGTT |
| | (p.Met1Val) | | | CACCCTGTCTCAGACCTCGAG′] |
| 4763 | NM_003159.2(CDKL5): c.211A > G | 6792 | CDKL5 | [′AATGCTTCGGACTCTCAAGCAGGAARACA |
| | (p.Asn71Asp) | | | TTGTGGAGTTGAAGGAAGCATT′] |
| 4764 | NM_003159.2(CDKL5): | 6792 | CDKL5 | [′AATTTTTTTTTTATCTTGACACTCCRGATAT |
| | c.404 − 2A > G | | | AAAACCAGAAAATCTCTTAA′] |
| 4765 | NM_003159.2(CDKL5): c.449A > G | 6792 | CDKL5 | [′TTAATCAGCCACAATGATGTCCTAARACTG |
| | (p.Lys150Arg) | | | TGTGACTTTGGTAAGTTAAAA′] |
| 4766 | NM_001813.2(CENPE): c.4063A > G | 1062 | CENPE | [′ACTTCAAGGGCTTCTTTTATCGTTTYAAGG |
| | (p.Lys1355Glu) | | | TTGTCTCTTTCCTTGGTTAGA′] |
| 4767 | NM_023073.3(C5orf42): | 65250 | C5orf42 | [′ATTTTACCAAATTTTTGTATTTGATRGATC |
| | c.3290 − 2A > G | | | CCATTGAAGAGGAAGATGCAA′] |
| 4768 | NM_002977.3(SCN9A): c.2215A > G | −1 | — | [′GTGTTTAAAACTATGCAAATGGTAAYTGC |
| | (p.Ile739Val) | | | AAGATCTACAAAAGGATCCATT′] |
| 4769 | NM_002354.2(EPCAM): | 4072 | EPCAM | [′TATTAGTATTAATTTGTATTATTCANTTTTT |
| | c.556 − 14A > G | | | TTCCCCAGTATGAGAATAAT′] |
| 4770 | NM_002354.2(EPCAM): | 4072 | EPCAM | [′TTAATACAGATTTTAAATTCTTTACRGTGC |
| | c.492 − 2A > G | | | ACTTCAGAAGGAGATCACAAC′] |
| 4771 | NM_000053.3(ATP7B): c.122A > G | 540 | ATP7B | [′ATCCAGACCACCTTCATAGCCAACAYTGTC |
| | (p.Asn41Ser) | | | AAAAGCAAAACTCTTCTTCAT′] |
| 4772 | NM_022455.4(NSD1): | 64324 | NSD1 | [′ATGTGGGACATTATTTTTTCTTTGCRAGGG |
| | c.4498 − 3A > G | | | AGAACTAATGCCTCACAGGAC′] |
| 4773 | NM_022455.4(NSD1): | 64324 | NSD1 | [′TAAGTCAGGAGGTATTTCTTGTTCTRGGGT |
| | c.5893 − 2A > G | | | GAATTTGTGAATGAGTATGTG′] |
| 4774 | NM_022455.4(NSD1): c.6059A > G | 64324 | NSD1 | [′AAAGGAAACTATGCTCGGTTCATGARTCA |
| | (p.Asn2020Ser) | | | TTGCTGCCAGCCCAACTGTGAA′] |
| 4775 | NM_022455.4(NSD1): c.6356A > G | 64324 | NSD1 | [′GGTGAAATCACAAAGGAGCGAGAAGRTGA |
| | (p.Asp2119Gly) | | | GTGTTTTAGTTGTGGGGATGCT′] |
| 4776 | NM_133433.3(NIPBL): c.737A > G | 25836 | NIPBL | [′AATCCTAGACATGGTTCAAGTGAGGRCTA |
| | (p.Asp246Gly) | | | CCTACACATGGTGCACAGGCTA′] |
| 4777 | NM_133433.3(NIPBL): | 25836 | NIPBL | [′TGTTTTTTTCTCTTCATTTTTCTTTRGCTTG |
| | 2c.5428 − A > G | | | ATATGCAACGAGGTGTTCAT′] |
| 4778 | NM_005249.4(FOXG1): c.757A > G | 2290 | FOXG1 | [′CCACTACGACGACCCGGGCAAGGGCRACT |
| | (p.Asn253Asp) | | | ACTGGATGCTGGACCCGTCGAG′] |
| 4779 | NM_004380.2(CREBBP): c.4508A > | 1387 | CREBBP | [′AAGCCAAAACGACTGCAGGAGTGGTRCAA |
| | G (p.Tyr1503Cys) | | | AAAGATGCTGGACAAGGCGTTT′] |
| 4780 | NM_004380.2(CREBBP): | 1387 | CREBBP | [′CTCACCTGCTCCTTCTGGACTTCCTRGGGC |
| | 2c.3983 − A > G | | | TGCAGACCACAAGACTGGGAA′] |
| 4781 | NM_001083962.1(TCF4): | 6925 | TCF4 | [′TTTTTTTTTTCTCCTTATTGTTTTARGATCT |
| | 2c.991 − A > G | | | ATTCTCCAGATCACACTAAC′] |
| 4782 | NM_178151.2(DCX): | 1641 | DCX | [′TTAACTTTGTCTCTTCTCTTCTTATRGGACC |
| | c.1027 − 2A > G | | | TGTACCTGCCTCTGTCCTTG′] |
| 4783 | NM_178151.2(DCX): c.607A > G | 1641 | DCX | [′TGTGCGTGTGCTTCTGAACAAGAAGRCAG |
| | (p.Thr203Ala) | | | CCCACTCTTTTGAGCAAGTCCT′] |
| 4784 | NM_178151.2(DCX): c.538A > G | 1641 | DCX | [′GGAGAACAAGGACTTTGTGCGCCCCRAGC |
| | (p.Lys180Glu) | | | TGGTTACCATCATCCGCAGTGG′] |
| 4785 | NM_178151.2(DCX): c.520A > G | 1641 | DCX | [′CAACAGTGCACAGGCCAGGGAGAACRAGG |
| | (p.Lys174Glu) | | | ACTTTGTGCGCCCCAAGCTGGT′] |
| 4786 | NM_178151.2(DCX): c.451A > G | 1641 | DCX | [′CAATCCCAACTGGTCTGTCAACGTADAAA |
| | (p.Lys151Glu) | | | CATCTGCCAATATGAAAGCCCC′] |
| 4787 | NM_178151.2(DCX): c.413A > G | 1641 | DCX | [′GACAACTTCTTTAAAAAGGTGGAGTRCAC |
| | (p.Tyr138Cys) | | | CAAGAATGTCAATCCCAACTGG′] |
| 4788 | NM_000252.2(MTM1): c.301A > G | 4534 | MTM1 | [′AATTGAAAAAATGGGAGGCGCGACARGTA |
| | (p.Ser101Gly) | | | GAGGAGAAAATTCCTATGGTCT′] |
| 4789 | NM_000252.2(MTM1): | 4534 | MTM1 | [′ACTGTCATACTTCTCCTTTGCCCCCRGGAC |
| | c.343 − 2A > G | | | ATGAGAAACCTGAGGTTCGCT′] |
| 4790 | NM_000252.2(MTM1): | 4534 | MTM1 | [′GACTTGAATTTCTTTTTTTCCTCACRGGGCT |
| | c.529 − 2A > G | | | TGCCCAATCACCATTGGAGA′] |
| 4791 | NM_000252.2(MTM1): c.550A > G | 4534 | MTM1 | [′ACAGGGCTTGCCCAATCACCATTGGRGAA |
| | (p.Arg184Gly) | | | TAACTTTTATTAATAAGTGCTA′] |
| 4792 | NM_000252.2(MTM1): c.629A > G | 4534 | MTM1 | [′TTGGTGGTTCCGTATCGTGCCTCAGRTGAT |
| | (p.Asp210Gly) | | | GACCTCCGGAGAGTTGCAACT′] |
| 4793 | NM_000252.2(MTM1): c.1406A > G | 4534 | MTM1 | [′CAATTTTTGATTATAATTTTGGATCRTCTGT |
| | (p.His469Arg) | | | ATAGTTGCCGATTTGGTACT′] |
| 4794 | NM_003159.2(CDKL5): c.62A > G | 6792 | CDKL5 | [′TTTGAGATCCTTGGGGTTGTAGGTGRAGGT |
| | (p.Glu21Gly) | | | AAGTTGGAATTTTTGCGTTCC′] |
| 4795 | NM_006306.3(SMC1A): c.3254A > | 8243 | SMC1A | [′GTGGCTACCAACATTGATGAGATCTRTAA |
| | G (p.Tyr1085Cys) | | | GGCCCTGTCCCGCAATAGCAGT′] |
| 4796 | NM_006306.3(SMC1A): | 8243 | SMC1A | [′CCTGCCACCATTCCCCTGTTTGCACRGGCT |
| | c.616 − 2A > G | | | GACCGGTACCAGCGCCTGAAG′] |
| 4797 | NM_003937.2(KYNU): c.592A > G | 8942 | KYNU | [′GCCTAACTTGATTTAGGGGGAAGAARCCT |
| | (p.Thr198Ala) | | | TAAGAATAGAGGATATCCTTGA′] |
| 4798 | NM_005343.2(HRAS): c.182A > G | −1 | — | [′TTGGACATCCTGGATACCGCCGGCCNGGA |
| | (p.Gln61Arg) | | | GGAGTACAGCGCCATGCGGGAC′] |
| 4799 | NM_012275.2(IL36RN): c.104A > G | 26525 | IL36RN | [′CTAGCTGGAGGGCTGCATGCAGGGARGGT |
| | (p.Lys35Arg) | | | CATTAAAGGTTGGTGATGAAAC′] |
| 4800 | NM_152296.4(ATP1A3): c.2318A > | 478 | ATP1A3 | [′TCCATTGCCTACACCCTGACCAGCADTATC |
| | G (p.Asn773Ser) | | | CCGGAGATCACGCCCTTCCTG′] |
| 4801 | NM_000891.2(KCNJ2): c.953A > G | 3759 | KCNJ2 | [′CAGTGCCGTAGCTCTTATCTAGCAARTGAA |
| | (p.Asn318Ser) | | | ATCCTGTGGGGCCACCGCTAT′] |
| 4802 | NM_017909.3(RMND1): c.713A > G | 55005 | RMND1 | [′TACACTTACAGTTTTGTCTTTCACAYTCCA |
| | (p.Asn238Ser) | | | AAACACAGCAGCTCCTTCCCT′] |
| 4803 | NM_005154.4(USP8): c.2150A > G | 9101 | USP8 | [′GAACCTTCCAAACTGAAGCGCTCCTDCTCC |
| | (p.Tyr717Cys) | | | TCCCCAGATATAACCCAGGCT′] |
| 4804 | NM_014191.3(SCN8A): c.667A > G | 6334 | SCN8A | [′CAATGTTTCAGCTCTACGCACTTTCRGGGT |
| | (p.Arg223Gly) | | | ACTGAGGGCTTTGAAAACTAT′] |
| 4805 | NM_021830.4(C10orf2): c.1754A > | 56652 | C10orf2 | [′CCCCAGGCAAGCCAGGAAGCAGACARTGT |
| | G (p.Asn585Ser) | | | TCTGATCCTGCAGGACAGGAAG′] |
| 4806 | NM_001164405.1(BHLHA9): | 727857 | BHLHA9 | [′GTCCAAGGCGCGGCGCATGGCCGCCRACG |
| | c.211A > G (p.Asn71Asp) | | | TGCGGGAGCGCAAGCGCATCCT′] |
| 4807 | NM_002887.3(RARS): c.5A > G | 5917 | RARS | [′GAGTGAGACGCTGATGGGAGGATGGRCGT |
| | (p.Asp2Gly) | | | ACTGGTGTCTGAGTGCTCCGCG′] |
| 4808 | NM_002887.3(RARS): c.1A > G | 5917 | RARS | [′TGGCGAGTGAGACGCTGATGGGAGGRTGG |
| | (p.Met1Val) | | | ACGTACTGGTGTCTGAGTGCTC′] |
| 4809 | NM_005861.3(STUB1): c.194A > G | 10273 | STUB1 | [′CCGCTGGTGGCCGTGTATTACACCARCCGG |
| | (p.Asn65Ser) | | | GCCTTGTGCTACCTGAAGATG′] |
| 4810 | NM_005211.3(CSF1R): | 1436 | CSF1R | [′ATGGGCATCCTCTGTCCTATCTCCCRGATA |
| | 2c.2655 − A > G | | | CAGCATCATGCAGGCCTGCTG′] |
| 4811 | NM_032228.5(FAR1): c.1094A > G | 84188 | FAR1 | [′CATAAGGCCCCAGCATTCCTGTATGRTATC |
| | (p.Asp365Gly) | | | TACCTCAGGATGACTGGAAGA′] |
| 4812 | NM_020461.3(TUBGCP6): | 85378 | TUBGCP6 | [′GCCATCCCAGGCAGGCGAGTGTTGCYCTG |
| | c.2546A > G (p.Glu849Gly) | | | CAGACCCAGAATCACAGCCTTG′] |
| 4813 | NM_014305.3(TGDS): c.269A > G | 23483 | TGDS | [′TTTGTGAAACTGCTTTTTGAAACAGRGAAA |
| | (p.Glu90Gly) | | | ATAGATATAGTACTACATTTT′] |
| 4814 | NM_014305.3(TGDS): c.892A > G | 23483 | TGDS | [′TCTGTTTTGTTTTCCAAGACCCACCRATGA |
| | (p.Asn298Asp) | | | CATGAGATACCCAATGAAGTC′] |
| 4815 | NM_000118.3(ENG): | 2022 | ENG | [′GCTCGACAGGATATTGACCACCGCCYGCG |
| | c.1273 − 2A > G | | | GGGATAAAGCCAGGGAGCTGGT′] |
| 4816 | NM_005360.4(MAF): c.172A > G | 4094 | MAF | [′CATCGCCGGGGGCTCGCTGTCCTCCRCCCC |
| | (p.Thr58Ala) | | | CATGAGCACGCCGTGCAGCTC′] |
| 4817 | NM_002238.3(KCNH1): c.1399A > | 3756 | KCNH1 | [′ACCTCTAGCACTTCTCTATGCCACCRTCTT |
| | G (p.Ile467Val) | | | CGGGAATGTGACGACTATTTT′] |
| 4818 | NM_172362.2(KCNH1): c.1508A > | 3756 | KCNH1 | [′TTCGGGAATGTGACGACTATTTTCCRACAG |
| | G (p.Gln503Arg) | | | ATGTATGCCAACACCAACAGA′] |
| 4819 | NM_003392.4(WNT5A): c.257A > G | 7474 | WNT5A | [′GGACAGAAGAAACTGTGCCACTTGTRTCA |
| | (p.Tyr86Cys) | | | GGACCACATGCAGTACATCGGA′] |
| 4820 | NM_001199252.2(SGOL1): c.67A > | −1 | — | [′GCCAAGTTTTTATTCCTTTTCTCTTYCATTC |
| | G (p.Lys23Glu) | | | GCTTCTTTATGTCTTCAAGA′] |
| 4821 | NM_000256.3(MYBPC3): | 4607 | MYBPC3 | [′ACTTAGCTACCCACTCTATACCCACRGAAC |
| | c.2906 − 2A > G | | | GGCCACGGCTTCAGCTGCCCA′] |
| 4822 | NM_000256.3(MYBPC3): | 4607 | MYBPC3 | [′GCTCAAGAATGGCCAGGAGATCCAGRTGA |
| | c.1213A > G (p.Met405Val) | | | GCGGCAGGTGCAGCCTGGGGTG′] |
| 4823 | NM_005188.3(CBL): | 867 | CBL | [′TGTTACTATCTTTTGCTTCTTCTGCRGGAAT |
| | c.1228 − 2A > G | | | CAGAAGGTCAGGGCTGTCCT′] |
| 4824 | NM_000257.3(MYH7): c.5326A > G | 4625 | MYH7 | [′TTCTTCATGCGCTCCAGGTGGGCGCYGGTG |
| | (p.Ser1776Gly) | | | TCCTGCTCCTTCTTCAGCTCC′] |
| 4825 | NM_000257.3(MYH7): c.2708A > G | 4625 | MYH7 | [′CAAGACAACCTGGCAGATGCTGAGGRGCG |
| | (p.Glu903Gly) | | | CTGTGATCAGCTGATCAAAAAC′] |
| 4826 | NM_000257.3(MYH7): c.1727A > G | 4625 | MYH7 | [′AATATCAAGGGGAAGCCTGAAGCCCRCTT |
| | (p.His576Arg) | | | CTCCCTGATCCACTATGCCGGC′] |
| 4827 | NM_000257.3(MYH7): c.1496A > G | 4625 | MYH7 | [′CACCACATGTTTGTGCTGGAGCAGGRGGA |
| | (p.Glu499Gly) | | | GTACAAGAAGGAGGGCATCGAG′] |
| 4828 | NM_000257.3(MYH7): c.2539A > G | 4625 | MYH7 | [′GCTGCTGAAGAGTGCAGAAAGAGAGRAGG |
| | (p.Lys847Glu) | | | AGATGGCCTCCATGAAGGAGGA′] |
| 4829 | NM_000257.3(MYH7): c.1954A > G | 4625 | MYH7 | [′CTTTCAGACTGTGTCAGCTCTGCACRGGGT |
| | (p.Arg652Gly) | | | GAGTGGGACACAGCCCCAGCC′] |
| 4830 | NM_000257.3(MYH7): c.1157A > G | 4625 | MYH7 | [′CTTCCAGAGGCTGACAAGTCTGCCTRCCTC |
| | (p.Tyr386Cys) | | | ATGGGGCTGAACTCAGCCGAC′] |
| 4831 | NM_000501.3(ELN): | 2006 | ELN | [′TACATTGCACTGTCCCCATCTCAACRGGTG |
| | c.800 − 2A > G | | | CTGGAGCAGCCGGAGTCCTCC′] |
| 4832 | NM_000117.2(EMD): | 2010 | EMD | [′TCTGCTACCGCTGCCCCCCTTCCCARGGCT |
| | c.266 − 2A > G | | | ACAATGACGACTACTATGAAG′] |
| 4833 | NM_000191.2(HMGCL): c.698A > G | 3155 | HMGCL | [′GTGCCTCTGGCTGCCCTGGCTGTCCRCTGC |
| | (p.His233Arg) | | | CATGACACCTATGGTCAAGCC′] |
| 4834 | NM_207352.3(CYP4V2): c.1393A > | 285440 | CYP4V2 | [′CTACGTGCCCTTCTCTGCTGGCCCCRGGAA |
| | G (p.Arg465Gly) | | | CTGTATAGGTTTGTATCCATC′] |
| 4835 | NM_013382.5(POMT2): | 29954 | POMT2 | [′GTCAGCAGGGTGGTCTCTATTCCACRGGGC |
| | 2c.1726 − A > G | | | CTACGCTTCTCAGGGGTCAAT′] |
| 4836 | NM_000169.2(GLA): | −1 | — | [′TTTGAATTATTTCATTCTTTTTCTCRGTTAG |
| | c.802 − 2A > G | | | TGATTGGCAACTTTGGCCTC′] |
| 4837 | NM_020166.4(MCCC1): | 56922 | MCCC1 | [′GTGTGATTTTCATGGTGTTTTAAACRGGAA |
| | c.137 − 2A > G | | | GAAACATTACCAAGGTCCTCA′] |
| 4838 | NM_006920.4(SCN1A): | 6323 | SCN1A | [′AATATATATTAATCTTTCATTTTCCRGCTG |
| | c.2557 − 2A > G | | | CGAGTTTTCAAGTTGGCAAAA′] |
| 4839 | NM_000501.3(ELN): | 2006 | ELN | [′CCTCACCCTCTGTGGCTGTGTTTTCRGGCG |
| | c.890 − 2A > G | | | TTGGGACTCCAGCTGCAGCTG′] |
| 4840 | NM_006306.3(SMC1A): | 8243 | SMC1A | [′TGACATTGCTGGGCCTGGGGCTTACRGGAT |
| | c.2974 − 2A > G | | | GCCCAGGCTGAGGAAGAGATC′] |
| 4841 | NM_001110792.1(MECP2): | 4204 | MECP2 | [′TGATTTTGACTTCACGGTAACTGGGRGAGG |
| | c.520A > G (p.Arg174Gly) | | | GAGCCCCTCCCGGCGAGAGCA′] |
| 4842 | NM_004646.3(NPHS1): c.1756A > G | 4868 | NPHS1 | [′CTTGTCCTGGGACAAGGAAGGGGAGRGGT |
| | (p.Arg586Gly) | | | GGGAGTGCGAGGGATCCCTCCC′] |
| 4843 | NM_003002.3(SDHD): c.275A > G | 6392 | SDHD | [′TATTTGAATCCTTGCTCTGCGATGGRCTAT |
| | (p.Asp92Gly) | | | TCCCTGGCTGCAGCCCTCACT′] |
| 4844 | NM_004793.3(LONP1): c.2353A > G | 9361 | LONP1 | [′CACGCTGTTTGTGGAGACATCCCTGRGACG |
| | (p.Arg785Gly) | | | GCCACAGGACAAGGATGCCAA′] |
| 4845 | NC_012920.1: m.4279A > G | 4565 | MT-TI | [′TCAAACCTAAGAAATATGTCTGATARAAG |
| | | | | AGTTACTTTGATAGAGTAAATA′] |
| 4846 | NM_005633.3(SOS1): c.1430A > G | 6654 | SOS1 | [′ATTTGCTGTAAATCAAATCATGGGCRGCCA |
| | (p.Gln477Arg) | | | AGACTTCCTGGTGCTAGCAAT′] |
| 4847 | NM_000256.3(MYBPC3): | 4607 | MYBPC3 | [′TGCCACTTCCCTGCGGCCCCCACCCRGGTA |
| | c.1227 − 2A > G | | | CATCTTTGAGTCCATCGGTGC′] |
| 4848 | NM_002834.3(PTPN11): c.661A > G | 5781 | PTPN11 | [′CGATCAGCCCCTTAACACGACTCGTDTAA |
| | (p.Ile221Val) | | | ATGCTGCTGAAATAGAAAGCAG′] |
| 4849 | NM_000257.3(MYH7): c.4664A > G | −1 | — | [′TCCCAGGCCTCCCTGGAGCACGAGGRGGG |
| | (p.Glu1555Gly) | | | CAAGATCCTCCGGGCCCAGCTG′] |
| 4850 | NM_000257.3(MYH7): c.2792A > G | 4625 | MYH7 | [′AACGAGAGGCTGGAGGATGAGGAGGRGA |
| | (p.Glu931Gly) | | | TGAATGCTGAGCTCACTGCCAAG′] |
| 4851 | NM_000257.3(MYH7): c.2087A > G | 4625 | MYH7 | [′CTGGTCATGCACCAGCTGCGCTGCARTGGT |
| | (p.Asn696Ser) | | | GTGCTGGAGGGCATCCGCATC′] |
| 4852 | NM_000257.3(MYH7): c.1805A > G | 4625 | MYH7 | [′CTGCAGAAGAACAAGGATCCTCTCARTGA |
| | (p.Asn602Ser) | | | GACTGTCGTGGGCTTGTATCAG′] |
| 4853 | NM_000257.3(MYH7): c.1615A > G | 4625 | MYH7 | [′CATGTCCATCCTGGAAGAGGAGTGCRTGTT |
| | (p.Met539Val) | | | CCCCAAGGCCACCGACATGAC′] |
| 4854 | NM_000257.3(MYH7): c.1477A > G | 4625 | MYH7 | [′GCTGCAGCAGTTCTTCAACCACCACRTGTT |
| | (p.Met493Val) | | | TGTGCTGGAGCAGGAGGAGTA′] |
| 4855 | NM_000257.3(MYH7): c.1331A > G | 4625 | MYH7 | [′TTCAACTGGATGGTGACGCGCATCAVTGC |
| | (p.Asn444Ser) | | | CACCCTGGAGACCAAGCAGCCA′] |
| 4856 | NM_000257.3(MYH7): c.1315A > G | 4625 | MYH7 | [′AGGGTGGCATTGATGCGCGTCACCAHCCA |
| | (p.Met439Val) | | | GTTGAACATCCTCTCATACACT′] |
| 4857 | NM_000257.3(MYH7): c.1051A > G | 4625 | MYH7 | [′TTCAGAGGAGAAAAACTCCATGTATRAGC |
| | (p.Lys351Glu) | | | TGACAGGCGCCATCATGCACTT′] |
| 4858 | NM_000257.3(MYH7): c.789A > G | 4625 | MYH7 | [′CAGGAAAGTTGGCATCTGCAGACATRGAG |
| | (p.Ile263Met) | | | ACCTGTGAGTGCCATGAATCTG′] |
| 4859 | NM_000257.3(MYH7): c.617A > G | 4625 | MYH7 | [′ATTGCAGCCATTGGGGACCGCAGCARGAA |
| | (p.Lys206Arg) | | | GGACCAGAGCCCGGGCAAGGTA′] |
| 4860 | NM_000363.4(TNNI3): c.616A > G | 7137 | TNNI3 | [′ACTGAGTGGAATGGAGGGCCGCAAGVAAA |
| | (p.Lys206Glu) | | | AGTTTGAGAGCTGAGCCTTCCT′] |
| 4861 | NM_000363.4(TNNI3): c.547A > G | 7137 | TNNI3 | [′GCAGGTGAAGAAGGAGGACACCGAGRAG |
| | (p.Lys183Glu) | | | GTGAGTGTGGGCTAAGGCCAGGA′] |
| 4862 | NM_000169.2(GLA): | −1 | — | [′TGACTCTTTTCCTCCCTCTCATTTCRGGTTC |
| | c.370 − 2A > G | | | ACAGCAAAGGACTGAAGCTA′] |
| 4863 | NM_000051.3(ATM): c.1A > G | 472 | ATM | [′GATGTGTGTTCTGAAATTGTGAACCVTGAG |
| | (p.Met1Val) | | | TCTAGTACTTAATGATCTGCT′] |
| 4864 | NM_000051.3(ATM): | 472 | ATM | [′TATTTAACCACAGTTCTTTTCCCGTRGGCT |
| | c.3154 − 2A > G | | | GATCCTTATTCAAAATGGGCC′] |
| 4865 | NM_024675.3(PALB2): | 79728 | PALB2 | [′AGTTATATACATTTTTTTCCTCCTCRGAAC |
| | c.212 − 2A > G | | | CTAAAAATAAAATATGTGTTT′] |
| 4866 | NM_024675.3(PALB2): | 79728 | PALB2 | [′TTCTGGGGCTGTTTTTGTCTCCTCTRGCGTG |
| | c.109 − 2A > G | | | CCCAAAGAGCTGAAAAGATT′] |
| 4867 | NM_000546.5(TP53): c.709A > G | 7157 | TP53 | [′CTGTACCACCATCCACTACAACTACRTGTG |
| | (p.Met237Val) | | | TAACAGTTCCTGCATGGGCGG′] |
| 4868 | NM_000455.4(STK11): c.889A > G | 6794 | STK11 | [′GATGCTTGAGTACGAACCGGCCAAGRGGT |
| | (p.Arg297Gly) | | | TCTCCATCCGGCAGATCCGGCA′] |
| 4869 | NM_017777.3(MKS1): c.1382A > G | 54903 | MKS1 | [′TCTCTGGAACTGGAGGACCTCTCCTRTGTA |
| | (p.Tyr461Cys) | | | CGGATACCAGGATCCTTCAAG′] |
| 4870 | NM_001231.4(CASQ1): c.731A > G | 844 | CASQ1 | [′ATGGAAGAGCCTGTGACCATCCCAGRCAA |
| | (p.Asp244Gly) | | | GCCCAATAGCGAAGAGGAGATT′] |
| 4871 | NM_001242896.1(DEPDC5): | 9681 | DEPDC5 | [′GTATGAGCAATCATCTGTTGTTTTCRGGAG |
| | c.2355 − 2A > G | | | GGACGAAGATGGTGTGCAGAT′] |
| | (p.Arg785_Gly839del) | | | |
| 4872 | NM_001039550.1(DNAJB2): | 3300 | DNAJB2 | [′TGACCAGTTGCCATGGCATCCTACTRCGAG |
| | c.14A > G (p.Tyr5Cys) | | | ATCCTAGACGTGCCGCGAAGT′] |
| 4873 | NM_007294.3(BRCA1): c.5057A > | 672 | BRCA1 | [′AATCTAATTACTGAAGAGACTACTCRTGTT |
| | G (p.His1686Arg) | | | GTTATGAAAACAGGTATACCA′] |
| 4874 | NM_006888.4(CALM1): c.389A > G | 801 | CALM1 | [′GTAGATGAAATGATCAGAGAAGCAGRTAT |
| | (p.Asp130Gly) | | | TGATGGAGACGGACAAGTCAAC′] |
| 4875 | NM_001082538.2(TCTN1): | 79600 | TCTN1 | [′TTGTATTATTATTTTTTTAATTTTCRGGGGC |
| | c.342 − 2A > G | | | GACAGCCAGTTTTGTAGTCA′] |
| 4876 | NM_003000.2(SDHB): | 6390 | SDHB | [′TCCTGCCTCTCTTTTCTCCCCATACRGGAC |
| | c.541 − 2A > G | | | GGGCTCTACGAGTGCATTCTC′] |
| 4877 | NM_000143.3(FH): c.700A > G | 2271 | FH | [′AGTGGAACAGCATCCTGAGTATGAGYACG |
| | (p.Thr234Ala) | | | TCCAATCTTGATGATCTGTGCA′] |
| 4878 | NM_000038.5(APC): | 324 | APC | [′ATAAAAACTTGTTTCTATTTTATTTRGAGC |
| | c.221 − 2A > G | | | TTAACTTAGATAGCAGTAATT′] |
| 4879 | NM_000314.6(PTEN): c.527A > G | 5728 | PTEN | [′ATTCCCAGTCAGAGGCGCTATGTGTRTTAT |
| | (p.Tyr176Cys) | | | TATAGCTACCTGTTAAAGAAT′] |
| 4880 | NM_030813.5(CLPB): c.1850A > G | 81570 | CLPB | [′CTGGTCGACGGCTACAATGTGCACTRTGGC |
| | (p.Tyr617Cys) | | | GCCCGCTCCATCAAACATGAG′] |
| 4881 | NM_030813.5(CLPB): c.1222A > G | 81570 | CLPB | [′TCCTGGAACTCGGACATGTCCAGCCHGAT |
| | (p.Arg408Gly) | | | GAAGCCCTGTGTGGAAACAAGC′] |
| 4882 | NM_016069.9(PAM16): c.226A > G | −1 | — | [′CTCACCCGTCCCCTCTCCTCTGCAGRACTA |
| | (p.Asn76Asp) | | | TGAACACTTATTTAAGGTGAA′] |
| 4883 | NM_058163.1(TSR2): c.191A > G | 90121 | TSR2 | [′TACTTAGCTGACTTGGAGCTAGATGRGGTG |
| | (p.Glu64Gly) | | | GAAGACTTCCTTGGAGAGCTG′] |
| 4884 | NM_001031.4(RPS28): c.1A > G | 6234 | RPS28 | [′ATAGGCTGCACACGGCTGGTGTCCAYGAT |
| | (p.Met1Val) | | | GGCGGCGCGGCGGCGGTCTGGC′] |
| 4885 | NM_005957.4(MTHFR): c.1114A > | 4524 | MTHFR | [′TCCCATCTTCTGGGCCTCCAGACCARAGAG |
| | G (p.Lys372Glu) | | | TTACATCTACCGTACCCAGGA′] |
| 4886 | NM_014946.3(SPAST): c.1165A > G | 6683 | SPAST | [′CTTTGGTCCACCTGGGAATGGGAAGRCAA |
| | (p.Thr389Ala) | | | TGCTGGTAAGGGTTCTCTTCAA′] |
| 4887 | NM_000228.2(LAMB3): | 3914 | LAMB3 | [′TAAATCCATAAGGTTAAGTTGGACCYACA |
| | 2c.565 − A > G | | | GAGGGAAGGGAAAGAGAAGCGC′] |
| 4888 | NM_000492.3(CFTR): | 1080 | CFTR | [′TCATTTACGTCTTTTGTGCATCTATDGGAG |
| | c.3368 − 2A > G | | | AAGGAGAAGGAAGAGTTGGTA′] |
| 4889 | NM_006785.3(MALT1): | 10892 | MALT1 | [′AACACCCCCTTTCTTTTTTTTTCAARGCGA |
| | c.1019 − 2A > G | | | AGGACAAGGTTGCCCTTTTGA′] |
| 4890 | NM_004771.3(MMP20): c.611A > G | 9313 | MMP20 | [′GGAGAAGGCCTGGGAGGAGATACACRTTT |
| | (p.His204Arg) | | | CGACAATGCTGAGAAGTGGACT′] |
| 4891 | NM_000918.3(P4HB): c.1178A > G | 5034 | P4HB | [′CACCCTAGAACTGCTTTCTTTTCAGRTGCC |
| | (p.Tyr393Cys) | | | CCATGGTGTGGTCACTGCAAA′] |
| 4892 | NM_177405.2(CECR1): c.355A > G | 51816 | CECR1 | [′TGCTCGCATCCCGCAGGCTCACCTGYTTCT |
| | (p.Thr119Ala) | | | CCGGCGTGGAAGAAGTAAGGC′] |
| 4893 | NM_004990.3(MARS): c.1031A > G | 4141 | MARS | [′TACCACATCATCCATGCTGACATCTRCCGC |
| | (p.Tyr344Cys) | | | TGGTTTAACATTTCGTTTGAT′] |
| 4894 | NM_025132.3(WDR19): | 57728 | WDR19 | [′TCTGTATAAAAATAATCTCTTTTTCRGGAA |
| | 2c.407 − A > G | | | AACATACTAAGAGAATCACTT′] |
| 4895 | NM_000314.6(PTEN): c.139A > G | 5728 | PTEN | [′TGCAGAAAGACTTGAAGGCGTATACRGGA |
| | (p.Arg47Gly) | | | ACAATATTGATGATGTAGTAAG′] |
| 4896 | NM_000314.6(PTEN): c.182A > G | 5728 | PTEN | [′GTTTTAAGGTTTTTGGATTCAAAGCDTAAA |
| | (p.His61Arg) | | | AACCATTACAAGATATACAAT′] |
| 4897 | NM_000314.6(PTEN): | 5728 | PTEN | [′TATTCTGAGGTTATCTTTTTACCACRGTTG |
| | c.254 − 2A > G | | | CACAATATCCTTTTGAAGACC′] |
| 4898 | NM_000314.6(PTEN): c.320A > G | 5728 | PTEN | [′GAACTTATCAAACCCTTTTGTGAAGRTCTT |
| | (p.Asp107Gly) | | | GACCAATGGCTAAGTGAAGAT′] |
| 4899 | NM_000314.6(PTEN): c.512A > G | 5728 | PTEN | [′CACCAGGGAGTAACTATTCCCAGTCRGAG |
| | (p.Gln171Arg) | | | GCGCTATGTGTATTATTATAGC′] |
| 4900 | NM_000314.6(PTEN): | 5728 | PTEN | [′TTTTCTTTTCTTTTTTTTTTTTTTTDGGACAA |
| | c.802 − 2A > G | | | AATGTTTCACTTTTGGGTA′] |
| 4901 | NM_001103.3(ACTN2): c.1883A > | 88 | ACTN2 | [′ATCCGCGATCAATCCCTGCAGGAGGRGCT |
| | G (p.Glu628Gly) | | | GGCTCGCCAGCATGCTAACGAG′] |
| 4902 | NM_003159.2(CDKL5): | 6792 | CDKL5 | [′GCTTTTAATTGTGTTTGTTTTTTTCRGGGAG |
| | c.−162 − 2A > G | | | TCATTTAATACTTCATGATT′] |
| 4903 | NM_003159.2(CDKL5): c.91A > G | 6792 | CDKL5 | [′AGCCTATGGAGTTGTACTTAAATGCRGAC |
| | (p.Arg31Gly) | | | ACAAGGCAAGTACATTATTTTT′] |
| 4904 | NM_003159.2(CDKL5): c.458A > G | 6792 | CDKL5 | [′CACAATGATGTCCTAAAACTGTGTGRCTTT |
| | (p.Asp153Gly) | | | GGTAAGTTAAAAAGAAATTAA′] |
| 4905 | NM_003159.2(CDKL5): | 6792 | CDKL5 | [′ATATGATAAAAATGTCTTCTCATTTRGGAA |
| | c.2277 − 2A > G | | | AAGTCCTGAAAATATTAGTCA′] |
| 4906 | NM_021098.2(CACNA1H): | 8912 | CACNA1H | [′CATCGTCAGCTTCTTCGTGCTCAACRTGTT |
| | c.4645A > G (p.Met1549Val) | | | CGTGGGCGTCGTGGTCGAGAA′] |
| 4907 | NM_003816.2(ADAM9): | 8754 | ADAM9 | [′TGTTTAATTTGAATTCTATTTCACTRGTTCC |
| | c.1396 − 2A > G | | | TTCCAGGAGGTACTTTATGC′] |
| 4908 | NM_212472.2(PRKAR1A): | 5573 | PRKAR1A | [′AATACTTTGAGAGGTTGGAGAAGGTRAAA |
| | c.177 +3 A > G | | | ATAAATGTGGGGAGATGATGAG′] |
| 4909 | NM_001204830.1(LIPT1): c.535A > | −1 | — | [′CCGGACTACTGCCTATCACCATTGCRCTTT |
| | G (p.Thr179Ala) | | | ATTATGTAGTACTGATGGGAC′] |
| 4910 | NM_001165963.1(SCN1A): | −1 | — | [′AACCCTGGAAGCTCAGTTAAGGGAGRCTG |
| | c.5264A > G (p.Asp1755Gly) | | | TGGGAACCCATCTGTTGGAATT′] |
| 4911 | NM_001165963.1(SCN1A): | −1 | — | [′TAATTGTTATTATTTTTGTGTGTGCRGGTTT |
| | c.3880 − 2A > G | | | CATTGGTCAGTTTAACAGCA′] |
| 4912 | NM_001165963.1(SCNIA): | 6323 | SCN1A | [′TTTATTGTGACGCTTAGCCTGGTAGRACTT |
| | c.2537A > G (p.Glu846Gly) | | | GGACTCGCCAATGTGGAAGGA′] |
| 4913 | NM_001165963.1(SCNIA): | 6323 | SCN1A | [′GTCATTGGATAGTGCTCCATGGCCAHGAA |
| | c.2353A > G (p.Met785Val) | | | AAGAGTATTTAAGACAATACAG′] |
| 4914 | NM_001165963.1(SCN1A): | 6323 | SCN1A | [′AGAGGTACTCCTCCCCACACCAGGTRTGG |
| | c.1662 + 3A > G | | | CACTGCTGAGTTTACTGATGCA′] |
| 4915 | NM_001165963.1(SCN1A): | 6323 | SCN1A | [′TGTGTGAAAGCTGGTAGAAATCCCADTTA |
| | c.1076A > G (p.Asn359Ser) | | | TGGCTACACAAGCTTTGATACC′] |
| 4916 | NM_001165963.1(SCN1A): | 6323 | SCN1A | [′TTGCAGCCAATGTCCAGAGGGATATRTGT |
| | c.1048A > G (p.Met350Val) | | | GTGTGAAAGCTGGTAGAAATCC′] |
| 4917 | NM_001165963.1(SCN1A): | 6323 | SCN1A | [′ACTTGCAGCCAATGTCCAGAGGGATRTAT |
| | c.1046A > G (p.Tyr349Cys) | | | GTGTGTGAAAGCTGGTAGAAAT′] |
| 4918 | NM_001165963.1(SCN1A): | 6323 | SCN1A | [′CACTATTTTGACAAACTGTGTGTTTRTGAC |
| | c.433A > G (p.Met145Val) | | | AATGAGTAACCCTCCTGATTG′] |
| 4919 | NM_001165963.1(SCN1A): | 6323 | SCN1A | [′TAGCTATTAAGATTTTGGTACATTCRTATC |
| | c.383 + 1A > G | | | CTTTTTCAAGTGATTAATATT′] |
| 4920 | NM_001605.2(AARS): c.2251A > G | 16 | AARS | [′TCGGCACCTGTGACAGCCACAATCCYCCG |
| | (p.Arg751Gly) | | | GATACCCTTGGCAATGGCTTCT′] |
| 4921 | NM_000124.3(ERCC6): | 2074 | ERCC6 | [′TATTCTCCATGCTCGCTCCCGGGCCYGCAA |
| | c.2830 − 2A > G | | | CAGAGAGAGAGAGACCTCTCA′] |
| 4922 | NM_000124.3(ERCC6): | 2074 | ERCC6 | [′ACTGGGAATGTGTATTTGCTTTGCAVACTC |
| | c.2599 − 26A > G | | | CTATCCCCCACCTCCAAACAG′] |
| 4923 | NM_001110556.1(FLNA): | 2316 | FLNA | [′ACTGAGGGGACTGGTGGCTGTTGTCRGGC |
| | c.1829 − 2A > G | | | TTCTCGGTGGAAGGGCCATCGC′] |
| 4924 | NM_006129.4(BMP1): c.808A > G | 649 | BMP1 | [′GGAGACCTATGACTTCGACAGCATCRTGC |
| | (p.Met270Val) | | | ATTACGCTCGGAACACATTCTC′] |
| 4925 | NM_001987.4(ETV6): c.1252A > G | 2120 | ETV6 | [′GGAGCCAGGACAAAGGCTTTTGTTCRGGT |
| | (p.Arg418Gly) | | | AGCACTTCCTTTTTCTCCTTTC′] |
| 4926 | NM_014423.3(AFF4): c.760A > G | 27125 | AFF4 | [′GTCCAATTCAATGTTACAGAAACCCRCTGC |
| | (p.Thr254Ala) | | | CTATGTGCGGCCCATGGACGG′] |
| 4927 | NM_002055.4(GFAP): c.1085A > G | 2670 | GFAP | [′AATGTCAAGCTGGCCCTGGACATCGRGAT |
| | (p.Glu362Gly) | | | CGCCACCTACAGGAAGCTGCTA′] |
| 4928 | NM_002055.4(GFAP): c.256A > G | 2670 | GFAP | [′TGACCGCTTTGCCAGCTACATCGAGRAGGT |
| | (p.Lys86Glu) | | | TCGCTTCCTGGAACAGCAAAA′] |
| 4929 | NM_006017.2(PROM1): | 8842 | PROM1 | [′ATTTGCTGTTCTTCACAGCTTTAGGRTATG |
| | c.2077 − 521A > G | | | TATCCAATGTTTTCTTCAGGT′] |
| 4930 | NM_002755.3(MAP2K1): c.305A > | 5604 | MAP2K1 | [′ACCTTTCTCCAGCTAATTCATCTGGRGATC |
| | G (p.Glu102Gly) | | | AAACCCGCAATCCGGAACCAG′] |
| 4931 | NM_001288953.1(TTC7A): | 57217 | TTC7A | [′CGTCCCCACAGCCTGATGTTCACCARGGTG |
| | Ac.1715 > G (p.Lys572Arg) | | | AAGCTGGAGCAGGTGCTGAAA′] |
| 4932 | NM_016218.2(POLK): c.181A > G | 51426 | POLK | [′TGAGCTCAAGAAAGAAAAGCAAGTCRACC |
| | (p.Asn61Asp) | | | AACGAATTGAAAATATGATGCA′] |
| 4933 | NM_016218.2(POLK): c.1385A > G | 51426 | POLK | [′AGAACTGTTACCATTAAGTTGAAGARTGT |
| | (p.Asn462Ser) | | | GAATTTTGAAGTAAAAACTCGT′] |
| 4934 | NM_016218.2(POLK): c.1477A > G | 51426 | POLK | [′ATTTGCCATTGCTAAGGAATTGCTARAAAC |
| | (p.Lys493Glu) | | | AGAAATTGATGCTGATTTTCC′] |
| 4935 | NM_001040431.2(COA3): c.215A > | 28958 | COA3 | [′GGAAATCGAGTAGAAGGTGTAACCAYCTG |
| | G (p.Tyr72Cys) | | | GGGAGGTAGGTTCAGGAAACCA′] |
| 4936 | NM_000891.2(KCNJ2): c.901A > G | 3759 | KCNJ2 | [′TGAAATCGTGGTCATACTGGAAGGCRTGG |
| | (p.Met301Val) | | | TGGAAGCCACTGCCATGACGAC′] |
| 4937 | NM_144499.2(GNAT1): c.386A > G | 2779 | GNAT1 | [′GACATCATCCAGCGGCTGTGGAAGGRCTC |
| | (p.Asp129Gly) | | | CGGTATCCAGGCCTGTTTTGAG′] |
| 4938 | NM_018965.3(TREM2): c.113A > G | 54209 | TREM2 | [′CAGTCCCTGCAGGTGTCTTGCCCCTRTGAC |
| | (p.Tyr38Cys) | | | TCCATGAAGCACTGGGGGAGG′] |
| 4939 | NM_194277.2(FRMD7): c.556A > G | 90167 | FRMD7 | [′ACTGGACATAGCAAGGAAGCTGGATRTGT |
| | (p.Met186Val) | | | ATGGCATCAGGCCTCACCCCGC′] |
| 4940 | NM_001003811.1(TEX11): | 56159 | TEX11 | [′TCACTCTCAACAGTAATCTTCTCCAYGGTC |
| | c.511A > G (p.Met171Val) | | | AAGTCAGCCTCAGGGGAGCTC′] |
| 4941 | NM_000921.4(PDE3A): c.1333A > G | 5139 | PDE3A | [′AGTTTCTTCCACTTGGACCACCACCRCCTC |
| | (p.Thr445Ala) | | | GGCCACAGGTCTACCCACCTT′] |
| 4942 | NM_000033.3(ABCD1): c.887A > G | 215 | ABCD1 | [′GCCAACTCGGAGGAGATCGCCTTCTRTGG |
| | (p.Tyr296Cys) | | | GGGCCATGAGGTGGGGCAGGTT′] |
| 4943 | NM_000169.2(GLA): c.137A > G | −1 | — | [′AGGACGCCTACCATGGGCTGGCTGCDCTG |
| | (p.His46Arg) | | | GGAGCGCTTCATGTGCAACCTT′] |
| 4944 | NM_000033.3(ABCD1): | 215 | ABCD1 | [′CCCTGACCCTGTCCCTCTCCTGGCCRGGAA |
| | c.1992 − 2A > G | | | ATACCACACACACTTGCTACA′] |
| 4945 | NM_000182.4(HADHA): | 3030 | HADHA | [′TTGCTCAATTCCAGTCTTTACCACCYAAAA |
| | c.919 − 2A > G | | | AACATATAAAGCACTTGCTCA′] |
| 4946 | NM_000159.3(GCDH): c.1213A > G | 2639 | GCDH | [′CGAGTATCACGTGATCCGGCACGCCRTGA |
| | (p.Met405Val) | | | ACCTGGAGGCCGTGAACACCTA′] |
| 4947 | NM_004006.2(DMD): | 1756 | DMD | [′ACAATTGTTAACTTCCTTCTTTGTCRGGGG |
| | c.1150 − 2A > G | | | TACATGATGGATTTGACAGCC′] |
| 4948 | NM_019109.4(ALG1): | 56052 | ALG | [′CTCAGGCTCCCTTGGTTCTCTCTGCRGTTT |
| | c.1188 − 2A > G | | | ACATGAGCTGGTGAAACATGA′] |
| 4949 | NM_004463.2(FGD1): | 2245 | FGD1 | [′TCTTTCTTTTTTATTCCCCACCCCARGGACT |
| | c.2016 − 2A > G | | | GAGGAGGAGAAGAAAGACTG′] |
| 4950 | NM_198525.2(KIF7): c.2981A > G | 374654 | KIF7 | [′CTGCTGCTGGCTCTGGGCGCTGCCCYGCCG |
| | (p.Gln994Arg) | | | CAGCTGCCCGCTCTTCTCGGA′] |
| 4951 | NM_020366.3(RPGRIP1): | 57096 | RPGRIP1 | [′ATGCTGTTTTTTTCCCTTTCCCAACRGTTGT |
| | c.3749 − 2A > G | | | TAGCCCTGAAGATCTGGCTA′] |
| 4952 | NM_003494.3(DYSF): c.3041A > G | 8291 | DYSF | [′GTTCCCCCTCCCCCAGGCTGGGAGTRTAGC |
| | (p.Tyr1014Cys) | | | ATCACCATCCCCCCGGAGCGG′] |
| 4953 | NM_003494.3(DYSF): | 8291 | DYSF | [′CTGCCATAACCAGCTTCGTGTCTCCRGGGC |
| | c.3349 − 2A > G | | | GGCGTGATGGATGACAAGAGT′] |
| 4954 | NM_004006.2(DMD): | 1756 | DMD | [′ATTACATTTCATTATAATTCTTTTCRGGTA |
| | c.4675 − 2A > G | | | ACAGAAAGAAAGCAACAGTTG′] |
| 4955 | NM_000169.2(GLA): c.620A > G | −1 | — | [′GTGTACTCCTGTGAGTGGCCTCTTTRTATG |
| | (p.Tyr207Cys) | | | TGGCCCTTTCAAAAGGTGAGA′] |
| 4956 | NM_022124.5(CDH23): | −1 | — | [′CTCTGCTCTCTCCCTTGGCTACTCCRGGTTC |
| | c.146 − 2A > G | | | TTCTGTGACCCAGTTGCTGG′] |
| 4957 | NM_000169.2(GLA): | −1 | — | [′GGGGTTGGAATGACCCAGATATGGTRAAA |
| | c.801 + 3A > G | | | ACTTGAGCCCTCCTTGTTCAAG′] |
| 4958 | NM_000019.3(ACAT1): c.473A > G | 38 | ACAT1 | [′GCAGGTGGGATGGAGAGCATGTCCARTGT |
| | (p.Asn158Ser) | | | TCCATATGTAATGAACAGAGGA′] |
| 4959 | NM_000553.4(WRN): c.561A > G | 7486 | WRN | [′GTCTGGTTAAACACCTCTTAGGTAARCAGC |
| | (p.Lys187=) | | | TCCTGAAAGACAAGTCTATCC′] |
| 4960 | NM_000050.4(ASS1): | 445 | ASS1 | [′GGCTCTGACCCCTTGTCCTATGTCCDGGTC |
| | c.421 − 2A > G | | | ATTGCTCCCTGGAGGATGCCT′] |
| 4961 | NM_020166.4(MCCC1): | 56922 | MCCC1 | [′TTCTGATCTAACAATCCTCATTCCCYAAGA |
| | c.640 − 2A > G | | | GAGAAAAGATGATTATGACTA′] |
| 4962 | NM_004006.2(DMD): | 1756 | DMD | [′TCTTTTTTACTTTTTTGATGCCAATRGGAAT |
| | c.10554 − 2A > G | | | CTGCAAGCAGAATATGACCG′] |
| 4963 | NM_017653.3(DYM): | 54808 | DYM | [′ACAAGTTTGCTGGTGTATGGAAGACYATA |
| | c.621 − 2A > G | | | CAAAAAGGAAAAAAAAATCAAA′] |
| 4964 | NM_001098398.1(COPA): c.728A > | −1 | — | [′TCAGAATCAAAGGCATGGGAGGTTGRTAC |
| | G (p.Asp243Gly) | | | CTGCCGGGGCCATTACAACAAT′] |
| 4965 | NM_005045.3(RELN): c.2288A > G | 5649 | RELN | [′CGTCAGCTAATTACATCTTTCCTTGRCAGC |
| | (p.Asp763Gly) | | | TCACAATCCAGGTGAGTGAAG′] |
| 4966 | NM_207111.3(RNF216): c.1616A > | 54476 | RNF216 | [′TGCCATCTCTTTGATTTTCTGCTCAHAGAA |
| | G (p.Tyr539Cys) | | | CTCCTGCTCTTGTTGCACAGC′] |
| 4967 | NM_001035.2(RYR2): c.568A > G | 6262 | RYR2 | [′CATCTTAGTTAGCGTGTCCTCTGAARGGTA |
| | (p.Arg190Gly) | | | CTTGGTAAGTGTGGAAAGTAG′] |
| 4968 | NM_001035.2(RYR2): c.11965A > G | 6262 | RYR2 | [′AAATGCAACTGCTTTACCACCAGGTVATGT |
| | (p.Asn3989Asp) | | | TGTTAATGGAACGATTGGCAA′] |
| 4969 | NM_001035.2(RYR2): c.12290A > G | 6262 | RYR2 | [′GAACCTGCGAAGGACATCGGCTTCARCGT |
| | (p.Asn4097Ser) | | | CGCCGTCCTTCTGACAAACCTC′] |
| 4970 | NM_001035.2(RYR2): c.12533A > G | 6262 | RYR2 | [′AGACAGTTCATATTTGACGTGGTCARCGA |
| | (p.Asn4178Ser) | | | AGGCGGAGAGAAAGAGAAGATG′] |
| 4971 | NM_000090.3(COL3A1): | 1281 | COL3A1 | [′ACAGTGACATGGCTTCTCTTTTTCCRGGGT |
| | c.2338 − 2A > G | | | GAAGGTGGTGCCCCCGGACTT′] |
| 4972 | NM_198056.2(SCN5A): c.4462A > G | 6331 | SCN5A | [′CTTAGGGGGCCAGGACATCTTCATGRCAG |
| | (p.Thr1488Ala) | | | AGGAGCAGAAGAAGTACTACAA′] |
| 4973 | NM_198056.2(SCN5A): c.4453A > G | 6331 | SCN5A | [′CTCTTTGCACTTAGGGGGCCAGGACRTCTT |
| | (p.Ile1485Val) | | | CATGACAGAGGAGCAGAAGAA′] |
| 4974 | NM_198056.2(SCN5A): | 6331 | SCN5A | [′GGTGAGCCTGACCCATTATCTCGACRGGTC |
| | c.2788 − 2A > G | | | CTGAATCTCTTCCTGGCCTTG′] |
| 4975 | NM_198056.2(SCN5A): c.1247A > G | 6331 | SCN5A | [′GATGGTGGCTTGGTTTTGCTCCTCAYAGGC |
| | (p.Tyr416Cys) | | | CATTGCGACCACGGCCAGGAT′] |
| 4976 | NM_004415.2(DSP): | 1832 | DSP | [′TTCACTGATCACTCTCATCCTTCACRGTTTT |
| | c.1141 − 2A > G | | | TTGAAGAGGCGCAGTCTACT′] |
| 4977 | NM_000238.3(KCNH2): c.2582A > | 3757 | KCNH2 | [′TGGTCCAGCCTGGAGATCACCTTCANCCTG |
| | G (p.Asn861Ser) | | | CGAGATGTGAGTTGGCTGCCC′] |
| 4978 | NM_000238.3(KCNH2): c.1913A > | 3757 | KCNH2 | [′GTCTCTCCCAACACCAACTCAGAGARGAT |
| | G (p.Lys638Arg) | | | CTTCTCCATCTGCGTCATGCTC′] |
| 4979 | NM_000238.3(KCNH2): c.1904A > | 3757 | KCNH2 | [′TTCGGCAACGTCTCTCCCAACACCADCTCA |
| | G (p.Asn635Ser) | | | GAGAAGATCTTCTCCATCTGC′] |
| 4980 | NM_000238.3(KCNH2): c.1900A > | 3757 | KCNH2 | [′GGGCTTCGGCAACGTCTCTCCCAACRCCAA |
| | G (p.Thr634Ala) | | | CTCAGAGAAGATCTTCTCCAT′] |
| 4981 | NM_000238.3(KCNH2): | 3757 | KCNH2 | [′CCACACCTCCGCCTTCCCCGGGTGCRGGTC |
| | c.1129 − 2A > G | | | CTGTCCCTGGGCGCCGACGTG′] |
| 4982 | NM_000238.3(KCNH2): c.296A > G | 3757 | KCNH2 | [′GAGCGCAAAGTGGAAATCGCCTTCTVCCG |
| | (p.Tyr99Cys) | | | GAAAGATGGTAGGAGCGGGCCG′] |
| 4983 | NM_000238.3(KCNH2): c.133A > G | 3757 | KCNH2 | [′GGAGAACTGCGCCGTCATCTACTGCDACG |
| | (p.Asn45Asp) | | | ACGGCTTCTGCGAGCTGTGCGG′] |
| 4984 | NM_001613.2(ACTA2): c.1A > G | 59 | ACTA2 | [′AGAATCCTGTGAAGCAGCTCCAGCTRTGT |
| | (p.Met1Val) | | | GTGAAGAAGAGGACAGCACTGC′] |
| 4985 | NM_000218.2(KCNQ1): c.605A > G | 3784 | KCNQ1 | [′GCTCCCCCTCTCCTGCACTCCACAGRCCTC |
| | (p.Asp202Gly) | | | ATCGTGGTCGTGGCCTCCATG′] |
| 4986 | NM_000218.2(KCNQ1): | 3784 | KCNQ1 | [′GGGAGCCTCCTGTCCATTCCTTCCCVGGGG |
| | c.1033 − 2A > G | | | ATTCTTGGCTCGGGGTTTGCC′] |
| 4987 | NM_000218.2(KCNQ1): | 3784 | KCNQ1 | [′CAATCTCCTCTCCTCTCTCCACTGCRGGCT |
| | c.1515 − 2A > G | | | GCGGGAACACCATCGGGCCAC′] |
| 4988 | NM_000218.2(KCNQ1): c.1787A > | 3784 | KCNQ1 | [′ATCGGCGCCCGCCTGAACCGAGTAGRAGA |
| | G (p.Glu596Gly) | | | CAAGGTAGGCTCACGCGCCGGC′] |
| 4989 | NM_000138.4(FBN1): c.7916A > G | 2200 | FBN1 | [′TGCATGTGTCCCGCCGGCTTCCAGTRTGAA |
| | (p.Tyr2639Cys) | | | CAGTTCAGTGGAGGATGCCAA′] |
| 4990 | NM_000138.4(FBN1) | 2200 | FBN1 | [′TTTTGCTTTTTTCTCCCTCCCCCCARGATAT |
| | c.4337 − 2A > G | | | TGATGAGTGCTCCCTTCCGA′] |
| 4991 | NM_000138.4(FBN1): c.3344A > G | 2200 | FBN1 | [′GGTCATTTCCATTTTGCAGATATTGRTGAG |
| | (p.Asp1115Gly) | | | TGTCAGAGAGATCCTCTCCTA′] |
| 4992 | NM_001943.3(DSG2): c.880A > G | 1829 | DSG2 | [′AGTCAACGTAGAAGTTACGCGCATARAAG |
| | (p.Lys294Glu) | | | TGTTCGATGCAGATGAAATAGG′] |
| 4993 | NM_001927.3(DES): c.1324A > G | 1674 | DES | [′TGAGCAAAGGGGTTCTGAGGTCCATRCCA |
| | (p.Thr442Ala) | | | AGAAGACGGTGATGATCAAGAC′] |
| 4994 | NM_004572.3(PKP2): | 5318 | PKP2 | [′AGAAATATGCATCTGCTTCTTCCCCRGGTT |
| | c.1171 − 2A > G | | | AACCAGCTTCGTGGCATCCTC′] |
| 4995 | NM_001256850.1(TTN): | −1 | — | [′GATATGAATATTTCACTCTTTTCTCRGGTC |
| | c.45629 − 2A > G | | | CTCCCTCACCACCCCTTGACC′] |
| 4996 | NM_013254.3(TBK1): c.1201A > G | 29110 | TBK1 | [′ACTTCATATTTCAGTTTCCCTCCCTRAAGT |
| | (p.Lys401Glu) | | | ACATCCACGTTATGATTTAGA′] |
| 4997 | NM_030973.3(MED25): c.116A > G | 81857 | MED25 | [′TACTTCGAGGGGCTCCGCAAGCACTRCCTG |
| | (p.Tyr39Cys) | | | CTCCCGGCCATCGAGTGAGTG′] |
| 4998 | NM_145207.2(SPATA5): c.1883A > | 166378 | SPATA5 | [′TCTATTTTTCAGGTATCCTGGTCAGRTATA |
| | G (p.Asp628Gly) | | | GGAGGACTGGAAAGTATCAAA′] |
| 4999 | NM_000016.5(ACADM): c.329A > | 34 | ACADM | [′ACTTTTGATGCTTGTTTAATTAGTGRAGAA |
| | G (p.Glu110Gly) | | | TTGGCTTATGGATGTACAGGG′] |
| 5000 | NM_000016.5(ACADM): c.797A > | 34 | ACADM | [′CCTAAAGAAAATGTTTTAATTGGTGRCGG |
| | G (p.Asp266Gly) | | | AGCTGGTTTCAAAGTTGCAATG′] |
| 5001 | NM_000532.4(PCCB): | 5096 | PCCB | [′GACTGTTCTGGAAATCTTTTATTTCRGGAC |
| | c.655 − 2A > G | | | ACCTCCTACCTGTTCATCACT′] |
| 5002 | NM_004453.3(ETFDH): c.929A > G | 2110 | ETFDH | [′CATACCTATGGAGGATCTTTCCTCTRTCAT |
| | (p.Tyr310Cys) | | | TTGAATGAAGGTGAACCCCTA′] |
| 5003 | NM_000255.3(MUT): c.1885A > G | 4594 | MUT | [′AAAAATGGGACAAGATGGCCATGACRGAG |
| | (p.Arg629Gly) | | | GAGCAAAAGTTATTGCTACAGG′] |
| 5004 | NM_000255.3(MUT): c.329A > G | 4594 | MUT | [′TTTAGGCCCTGGACCATCCGCCAGTRTGCT |
| | (p.Tyr110Cys) | | | GGTTTTAGTACTGTGGAAGAA′] |
| 5005 | NM_000017.3(ACADS): c.1108A > | 35 | ACADS | [′TCAGGCCATCCAGATCCTGGGCGGCRTGG |
| | G (p.Met370Val) | | | GCTACGTGACAGAGATGCCGGC′] |
| 5006 | NM_174917.4(ACSF3): c.1A > G | 197322 | ACSF3 | [′TCCAGCTCGGCCGCCTGTCAGTGCARTGCT |
| | (p.Met1Val) | | | GCCCCATGTGGTGCTCACCTT′] |
| 5007 | NM_000531.5(OTC): c.919A > G | 5009 | OTC | [′GACATTTTTACACTGCTTGCCCAGARAGCC |
| | (p.Lys307Glu) | | | AGAAGAAGTGGATGATGAAGT′] |
| 5008 | NM_000030.2(AGXT): c.248A > G | 189 | AGXT | [′ACACTGGTCATCTCTGGCTCGGGACRCTGT |
| | (p.His83Arg) | | | GCCCTGGAGGCCGCCCTGGTC′] |
| 5009 | NM_000030.2(AGXT): | 189 | AGXT | [′CACCCACAGCCGTCCCTGCTTCCTCRGGGC |
| | c.424 − 2A > G | | | CTGGCCCAGCACAAGCCAGTG′] |
| | (p.Gly_142Gln145del) | | | |
| 5010 | NM_000030.2(AGXT): | 189 | AGXT | [′CGTCCCGAGCAAACCACCCATCTACRGGC |
| | c.596 − 2A > G | | | ATCGACATCCTGTACTCGGGCT′] |
| 5011 | NM_000030.2(AGXT): | 189 | AGXT | [′TGGACCAAGCCCCCTCGTGTCTTCCRGGTA |
| | c.777 − 2A > G | | | CCATCACACAATCCCCGTCAT′] |
| 5012 | NM_012203.1(GRHPR): | 9380 | GRHPR | [′CTCCTGAGGGCCTCCCTTTCCCCGCRGCTG |
| | c.84 − 2A > G | | | TGAGGTGGAGCAGTGGGACTC′] |
| 5013 | NM_012203.1(GRHPR): c.934A > G | 9380 | GRHPR | [′CACCATGTCCTTGTTGGCAGCTAACRACTT |
| | (p.Asn312Asp) | | | GCTGGCTGGCCTGAGAGGGGA′] |
| 5014 | NM_203290.2(POLRIC): c.221A > | 9533 | POLRIC | [′GTGGGAATTGACGCAGCCATTGCCARTGC |
| | G (p.Asn74Ser) | | | TTTTCGACGAATTCTGCTAGCT′] |
| 5015 | NM_006516.2(SLC2A1): c.848A > G | 6513 | SLC2A1 | [′GCTGTGGTGCTGCAGCTGTCCCAGCRGCTG |
| | (p.Gln283Arg) | | | TCTGGCATCAACGCTGTGAGT′] |
| 5016 | NM_006516.2(SLC2A1): | 6513 | SLC2A1 | [′ATAACAGTGTGGTTTGTTTCTCCGCRGAAG |
| | c.19 − 2A > G | | | CTGACGGGTCGCCTCATGCTG′] |
| 5017 | NM_021007.2(SCN2A): | 6326 | SCN2A | [′ACTTTGTCTTCCTTGACGATATTCTRCTTTA |
| | c.387 − 2A > G | | | TTCAATATGCTCATTATGTG′] |
| 5018 | NM_021007.2(SCN2A): c.851A > G | 6326 | SCN2A | [′AATAAATGTTTGCAATGGCCTCCAGRTAAT |
| | (p.Asp284Gly) | | | TCTTCCTTTGAAATAAATATC′] |
| 5019 | NM_021007.2(SCN2A): c.4036A > G | 6326 | SCN2A | [′CATGAATGTACTTCTGGTTTGTCTGRTCTTT |
| | (p.Ile1346Val) | | | TGGCTAATATTCAGTATCAT′] |
| 5020 | NM_001165963.1(SCN1A): | −1 | — | [′TAATTTCAACCAGCAGAAAAAGAAGDTAA |
| | c.4476 + 1A > G | | | GTATTTCTAATATTTTCTCTCC′] |
| 5021 | NM_001165963.1(SCN1A): | 6323 | SCN1A | [′ATCCTGGCTGTGGTGGCCATGGCCTRCGAG |
| | c.1277A > G (p.Tyr426Cys) | | | GAACAGAATCAGGCCACCTTG′] |
| 5022 | NM_000391.3(TPP1): c.833A > G | 1200 | TPP1 | [′GGGATTGAGGCCAGTCTAGATGTGCRGTA |
| | (p.Gln278Arg) | | | CCTGATGAGTGCTGGTGCCAAC′] |
| 5023 | NM_017882.2(CLN6): c.767A > G | 54982 | CLN6 | [′GGAGAAGAGGAAGAGGCCGTTGCTGYCCA |
| | (p.Asp256Gly) | | | GGAAGAGGCGCTTGCGCTTCTG′] |
| 5024 | NM_002693.2(POLG): c.3470A > G | 5428 | POLG | [′GGGCCCCGCATACCTGGTCAAGAGGYTGG |
| | (p.Asn1157Ser) | | | TGATCTGCAAGGCCAGGGCAGC′] |
| 5025 | NM_002693.2(POLG): c.2840A > G | 5428 | POLG | [′GTGGGCATCAGCCGTGAGCATGCCARAAT |
| | (p.Lys947Arg) | | | CTTCAACTACGGCCGCATCTAT′] |
| 5026 | NM_002693.2(POLG): c.2636A > G | 5428 | POLG | [′CACAAGGGTGTAGCCAGGTGGGGCCYGCA |
| | (p.Gln879Arg) | | | CCATGGCTTTCAACTCACTGCC′] |
| 5027 | NM_000833.4(GRIN2A): c.2449A > | 2903 | GRIN2A | [′GAGCAGCCAGCTGGACATTGACAACRTGG |
| | G (p.Met817Val) | | | CGGGCGTATTCTACATGCTGGC′] |
| 5028 | NM_000833.4(GRIN2A): c.1930A > | 2903 | GRIN2A | [′CTTCTTCGCTGTCATATTCCTGGCTRGCTA |
| | G (p.Ser644Gly) | | | CACAGCCAATCTGGCTGCCTT′] |
| 5029 | NM_000156.5(GAMT): c.1A > G | 2593 | GAMT | [′GTCGGGTCGCCGTCCAGCCTGCAGCRTGA |
| | (p.Met1Val) | | | GCGCCCCCAGCGCGACCCCCAT′] |
| 5030 | NM_172107.2(KCNQ2): c.848A > G | 3785 | KCNQ2 | [′CTGACCACCATTGGCTACGGGGACARGTA |
| | (p.Lys283Arg) | | | CCCCCAGACCTGGAACGGCAGG′] |
| 5031 | NM_172107.2(KCNQ2): c.611A > G | 3785 | KCNQ2 | [′GCGCTCCGGAGCCTGCGCTTCCTGCRGATT |
| | (p.Gln204Arg) | | | CTGCGGATGATCCGCATGGAC′] |
| 5032 | NM_172107.2(KCNQ2): | 3785 | KCNQ2 | [′CTGAGAGCGCGCGTTCCCTGCCCCCRGGTT |
| | c.297 − 2A > G | | | CCTCCTGGTTTTCTCCTGCCT′] |
| 5033 | NM_001105243.1(PCDH19): | 57526 | PCDH19 | [′GTCACCGTCAGCGTGCTGGACACCARTGA |
| | c.1019A > G (p.Asn340Ser) | | | CAATCCGCCGGTCATCAACCTG′] |
| 5034 | NM_001105243.1(PCDH19): | 57526 | PCDH19 | [′CTTAGTATCAAGGTGACCGACTCCADTGA |
| | c.695A > G (p.Asn232Ser) | | | CAACAACCCGGTGTTTAGCGAG′] |
| 5035 | NM_052859.3(RFT1): c.454A > G | 91869 | RFT1 | [′TTCACAGCAGAAGGTGCACTGACCTBGAG |
| | (p.Lys152Glu) | | | CTTCACAAACATATGTGCTTGT′] |
| 5036 | NM_052859.3(RFT1): c.1222A > G | 91869 | RFT1 | [′TCTCCTCCCCAGGTACAATTTTGTGRTGCT |
| | (p.Met408Val) | | | GGCCCTGTCCTCCTCATTCCT′] |
| 5037 | NM_020533.2(MCOLN1): | 57192 | MCOLN1 | [′TCTGAGTGCCTGTTCTCGCTCATCARTGGG |
| | c.1406A > G (p.Asn469Ser) | | | GACGACATGTTTGTGACGTTC′] |
| 5038 | NM_004456.4(EZH2): c.458A > G | 2146 | EZH2 | [′TTCATTGAAGAACTAATAAAAAATTRTGAT |
| | (p.Tyr153Cys) | | | GGGAAAGTACACGGGGATAGA′] |
| 5039 | NM_005045.3(RELN): c.2168A > G | 5649 | RELN | [′ACCACGGATAGAGTAAAAGTTATGGYAAG |
| | (p.Tyr723Cys) | | | AGGAGAGCCTGGAACTGCCAAA′] |
| 5040 | NM_006772.2(SYNGAP1): | 8831 | SYNGAP1 | [′CCCACCCCATCCCCATTTCCCCCCCRGCAA |
| | c.388 − 2A > G | | | GGCTTCCTGAGCCGACGGCTA′] |
| 5041 | NM_001080508.2(TBX18): | 9096 | TBX18 | [′GATAGGCACTGAGATGATCATCACCRAGG |
| | c.487A > G (p.Lys163Glu) | | | CCGGCAGGTAATGGGCAAGCTG′] |
| 5042 | NM_004964.2(HDAC1): c.461A > G | 3065 | HDAC1 | [′GAGGCATCTGGCTTCTGTTACGTCARTGAT |
| | (p.Asn154Ser) | | | ATCGTCTTGGCCATCCTGGAA′] |
| 5043 | NM_014946.3(SPAST): c.1168A > G | 6683 | SPAST | [′TGGTCCACCTGGGAATGGGAAGACARTGC |
| | (p.Met390Val) | | | TGGTAAGGGTTCTCTTCAAATT′] |
| 5044 | NM_000314.6(PTEN): c.71A > G | 5728 | PTEN | [′TATCAAGAGGATGGATTCGACTTAGRCTTG |
| | (p.Asp24Gly) | | | ACCTGTATCCATTTCTGCGGC′] |
| 5045 | NM_004380.2(CREBBP): c.4409A > | 1387 | CREBBP | [′GGGTCCTGCAGGTATGTGACAGGGCRCAT |
| | G (p.His1470Arg) | | | CTGGGCCTGTCCTCCAAGTGAA′] |
| 5046 | NM_018206.4(VPS35): c.1463A > G | 55737 | VPS35 | [′GATCCAGAAGATTTTGCTGATGAGCRGAG |
| | (p.Gln488Arg) | | | CCTTGTGGGCCGCTTCATTCAT′] |
| 5047 | NM_152296.4(ATP1A3): c.971A > G | 478 | ATP1A3 | [′GGCATCATCGTGGCCAATGTCCCAGRGGG |
| | (p.Glu324Gly) | | | TCTGCTGGCCACTGTCACTGTA′] |
| 5048 | NM_172107.2(KCNQ2): c.710A > G | 3785 | KCNQ2 | [′TCTCAGGAGCTGGTCACTGCCTGGTRCATC |
| | (p.Tyr237Cys) | | | GGCTTCCTTTGTCTCATCCTG′] |
| 5049 | NM_001017980.3(VMA21): | 203547 | VMA21 | [′ACTAAATCTTACATATTTGAAGGTARTCTT |
| | c.163 + 4A > G | | | AGACCCATTAAAACAAGATGT′] |
| 5050 | NM_000489.4(ATRX): c.740A > G | 546 | ATRX | [′TTCTGCAAGAAATGCATTCTACGCARCCTT |
| | (p.Asn247Ser) | | | GGTCGAAAGGAGTTGTCCACA′] |
| 5051 | NM_021147.4(CCNO): c.716A > G | 10309 | CCNO | [′CCCACCATTAGCTTCTTCCTGGAGCRTTTC |
| | (p.His239Arg) | | | ACGCACGCTCGCGTGGAGGCG′] |
| 5052 | NM_031924.4(RSPH3): | 83861 | RSPH3 | [′CTCTAGAGAATCAGGCCGTCCGAGCYAAC |
| | c.631 − 2A > G | | | AGTGATAGAAAATACTTCTAGA′] |
| 5053 | NM_000069.2(CACNA1S): | 779 | CACNAIS | [′TCGCTTTCCCATCCTTTTCCTTCCCRGGGCT |
| | c.3526 − 2A > G | | | ACTTTGGAGACCCCTGGAAT′] |
| 5054 | NM_000533.4(PLP1): c.1A > G | 5354 | PLP1 | [′CAATTGGAGTCAGAGTCCCAAAGACRTGG |
| | (p.Met1Val) | | | GTAAGTTTCAAAAACTTTAGCA′] |
| 5055 | NM_207352.3(CYP4V2): c.1396A > | 285440 | CYP4V2 | [′CGTGCCCTTCTCTGCTGGCCCCAGGRACTG |
| | G (p.Asn466Asp) | | | TATAGGTTTGTATCCATCTGA′] |
| 5056 | NM_033409.3(SLC52A3): c.403A > | 113278 | SLC52A3 | [′ACAAAGAAGGTGGTGAGGTAGTAGGYGG |
| | G (p.Thr135Ala) | | | GCAGCCGGCTCATGAACGGCAGG′] |
| 5057 | NM_033409.3(SLC52A3): c.62A > G | 113278 | SLC52A3 | [′CAGGGGCAGCTCTACCCAGAGCCCAYTGA |
| | (p.Asn21Ser) | | | TGGTCACCCAGGAGCCCATTCC′] |
| 5058 | NM_017662.4(TRPM6): c.3173A > | 140803 | TRPM6 | [′CAAGCTGTCTACCTCTTCGTGCAATRTATC |
| | G (p.Tyr1058Cys) | | | ATCATGGTGAACCTGTTGATT′] |
| 5059 | NM_006642.3(SDCCAG8): | 10806 | SDCCAG8 | [′AATAAACCCTCTGCTTTTGCTCTATRGTTA |
| | c.221 − 2A > G | | | ATCAGCTCAAAGATTTGTTGC′] |
| 5060 | NM_022455.4(NSD1): c.5990A > G | 64324 | NSD1 | [′CAAGAACATGATATCACTAATTTCTRTATG |
| | (p.Tyr1997Cys) | | | CTCACCCTAGACAAAGTAAGT′] |
| 5061 | NM_000352.4(ABCC8): c.563A > G | 6833 | ABCC8 | [′GGGATGCTGCTCCTCGTGGAGGTCARTGTC |
| | (p.Asn188Ser) | | | ATCAGGGTGAGGGTAAGCAGG′] |
| 5062 | NM_000275.2(OCA2): c.1427A > G | 4948 | OCA2 | [′GATGGCAGTGGCAGCTCCTCCAATGYTTGT |
| | (p.Asn476Ser) | | | GAAGATCACTTCTGCAATCAG′] |
| 5063 | NM_001848.2(COL6A1): | 1291 | COL6A1 | [′ATTTTCTAGTTTTCTTCCTCTTTCCRGGGGG |
| | c.1003 − 2A > G | | | AGATGGGGTACCCAGGCCTG′] |
| 5064 | NM_003560.2(PLA2G6) | 8398 | PLA2G6 | [′CAGCATGCCCTGCTCTGTGCCTCACRGAAC |
| | c.1349 − 2A > G | | | TACAGGATCTCATGCACATCT′] |
| 5065 | NM_178151.2(DCX): c.280A > G | 1641 | DCX | [′TGACCTGACGCGATCTCTGTCTGACRACAT |
| | (p.Asn94Asp) | | | CAACCTGCCTCAGGGAGTGCG′] |
| 5066 | NM_000425.4(L1CAM): c.2351A > | 3897 | LICAM | [′TCCAACACGTCCACCTTCGTGCCCTRTGAG |
| | G (p.Tyr784Cys) | | | ATCAAAGTCCAGGCCGTCAAC′] |
| 5067 | NM_000052.6(ATP7A): | 538 | ATP7A | [′TCAATGATTATCATTCCTATATTGCRGTTTT |
| | c.2173 − 2A > G | | | TCGGAGGCTGGTACTTCTAC′] |
| 5068 | NM_020247.4(ADCK3): c.1286A > | 56997 | ADCK3 | [′CTGCTGAAGGGCCACCCCTTCTTCTRTGTG |
| | G (p.Tyr429Cys) | | | CCTGAGATTGTGGATGAGCTC′] |
| 5069 | NM_000143.3(FH): c.554A > G | 2271 | FH | [′AAACAGCAAAGCTCACATACTGACCYGGC |
| | (p.Gln185Arg) | | | TTTTATTAACATGATCGTTGGG′] |
| 5070 | NM_033109.4(PNPT1): c.1453A > G | 87178 | PNPT1 | [′AAACTTCCGCCACATGCAGATGCCAKAGA |
| | (p.Met485Val) | | | AGATGACCCTATAGAAAGAAAA′] |
| 5071 | NM_005888.3(SLC25A3): | 5250 | SLC25A3 | [′TACTTACTTGATTTTTTTTTTTCCARTCAAA |
| | c.158 − 9A > G | | | CAGAGCAGTATAGCTGTGAC′] |
| 5072 | NM_181426.1(CCDC39): | 339829 | CCDC39 | [′TTGTGCTGCTTTATCCAATTCTAACYGTCA |
| | c.610 − 2A > G | | | AACAGAGAGCAAAGAACATTT′] |
| 5073 | NM_153704.5(TMEM67): c.725A > | 91147 | TMEM67 | [′ATTGTTCTGTTGTAGGTATATGCCARTCTA |
| | G (p.Asn242Ser) | | | ACATCTTGTCAAGCTCTTGGA′] |
| 5074 | NM_000166.5(GJB1): c.305A > G | 2705 | GJB1 | [′CACGTGGCTCACCAGCAACACATAGRGAA |
| | (p.Glu102Gly) | | | GAAAATGCTACGGCTTGAGGGC′] |
| 5075 | NM_019098.4(CNGB3): c.1193A > | 54714 | CNGB3 | [′AATTAAAGTTCGAACTGCCCAATAAYAAC |
| | G (p.Tyr398Cys) | | | ATCTCAGATACCTGTGAAAACA′] |
| 5076 | NM_000070.2(CAPN3): | 825 | CAPN3 | [′CCATATGGCTCTCTCTCTTCTTCCARCCTCT |
| | c.1194 − 9A > G | | | CAGGATGTCCTATGAGGATT′] |
| 5077 | NM_002860.3(ALDH18A1): | 5832 | ALDH18A1 | [′AGGAATAGGGAGGTTCTCATGAAGABATT |
| | c.2345A > G (p.Tyr782Cys) | | | TTAAACTTCCATGCTCTGAGAA′] |
| 5078 | NM_001414.3(EIF2B1): c.824A > G | 1967 | EIF2B1 | [′CAGAGTGATTAAGGAAGGGGCAGTGYAGT |
| | (p.Tyr275Cys) | | | CGACCCACGGATGCTCCTCTTT′] |
| 5079 | NM_182896.2(ARL13B): c.461A > G | 200894 | ARL13B | [′TGTCTATCTCTGGAAAAATTGGTCARTGAG |
| | (p.Asn154Ser) | | | CACAAGTGCCTGTGTCAGATA′] |
| 5080 | NM_001134831.1(AHI1): | 54806 | AHI1 | [′TAGTAAGATGAAACAGGCCGTCCACYGTA |
| | c.1152 − 2A > G | | | CAAAAAAAGATACTTCCATTAA′] |
| 5081 | NM_153704.5(TMEM67): | 91147 | TMEM67 | [′TCAGTTTTAAAGGAGAAAACCAGGTRAAA |
| | c.978 + 3A > G | | | GTGTCTAATATCATTAGAGGAT′] |
| 5082 | NM_015681.3(B9D1): c.95A > G | 27077 | B9D1 | [′CCAGTCCTGGCCGTACACAAAGCAGYACT |
| | (p.Tyr32Cys) | | | TGCAGTAGAGGTCATCATACTC′] |
| 5083 | NM_014049.4(ACAD9): c.1A > G | 28976 | ACAD9 | [′CTGAGGCTGGGGAACATCGGGCAGCWTGA |
| | (p.Met1Val) | | | GCGGCTGCGGGCTCTTCCTGCG′] |

TABLE 8
xCas9v3 Mutations (K294R/Q1256K Series)

| xCas9 3.0 | xCas9 3.1 | xCas9 3.2 | xCas9 3.3 | xCas9 3.4 |
|---|---|---|---|---|
| | | N175T | | |
| P230F | | | | |
| | | | D257N | |
| S267G | | S267G | S267G | |
| K294R | K294R | K294R | K294R | K294R |
| | | | T466A | |
| E480K | E480K | E480K | E480K | E480K |
| E543D | E543D | E543D | E543D | E543D |
| | A711E | | | |
| | E1207G | | I1063V | |
| E1219V | E1219V | E1219V | E1219V | E1219V |
| Q1256K | Q1256K | Q1256K | Q1256K | Q1256K |

TABLE 9
xCas9v3 Mutations (A262T/S409I Series)

| xCas9 3.5 | xCas9 3.6 | xCas9 3.7 | xCas9 3.8 | xCas9 3.9 | xCas9 3.10 | xCas9 3.11 | xCas9 3.12 |
|---|---|---|---|---|---|---|---|
| E108G | E108G | | | | | E108G | E108G |
| | S217A | | | | | | |
| A262T | A262T | A262T | A262T | A262T | A262T | A262T | A262T |
| | | R324L | | | | | |
| S409I | S409I | S409I | S409I | S409I | S409I | S409I | S409I |
| E480K | E480K | E480K | E480K | E480K | E480K | E480K | E480K |
| E543D | E543D | E543D | E543D | E543D | E543D | E543D | E543D |
| | | | D605D | D605D | | | |
| K673E | | | | | | K673E | |
| M694I | M694I | M694I | M694I | M694I | M694I | M694I | M694I |
| E1219V | E1219V | E1219V | E1219V | E1219V | E1219V | E1219V | E1219V |
| | | | H1264Y | | | | |
| L1365I | L1365I | | | | L1365I | | L1365I |

TABLE 10
PAM Depletion Scores (xCas9v3.0-3.6 Mutations)

| | 22-4 | 22-10 | 22-11 | 22-14 | 22-21 | 22-65 | 22-66 |
|---|---|---|---|---|---|---|---|
| | xCas9 | xCas9 | xCas9 | xCas9 | xCas9 | xCas9 | xCas9 |
| | 3.0 | 3.1 | 3.2 | 3.3 | 3.4 | 3.5 | 3.6 |
| AAA | 0.61 | 0.69 | 1.38 | 0.90 | 0.98 | 1.29 | 1.47 |
| AAC | 0.67 | 0.77 | 0.78 | 0.71 | 0.93 | 0.80 | 1.55 |
| AAG | 13.88 | 15.70 | 32.70 | 28.75 | 22.79 | 48.60 | 92.02 |
| AAT | 0.52 | 0.59 | 0.87 | 0.63 | 0.76 | 0.87 | 0.84 |
| ACA | 0.52 | 0.52 | 0.48 | 0.51 | 0.57 | 0.59 | 0.72 |
| ACC | 0.54 | 0.50 | 0.48 | 0.48 | 0.47 | 0.46 | 0.55 |
| ACG | 10.15 | 30.07 | 41.38 | 108.50 | 32.19 | 25.52 | 158.44 |
| ACT | 0.46 | 0.47 | 0.39 | 0.41 | 0.44 | 0.40 | 0.50 |
| AGA | 13.22 | 28.30 | 34.42 | 57.31 | 38.26 | 31.93 | 170.85 |
| AGC | 7.55 | 5.84 | 11.17 | 8.69 | 7.63 | 13.13 | 5.54 |
| AGG | 12.23 | 188.82 | 37.97 | 182.49 | 194.93 | 82.99 | 355.32 |
| AGT | 13.19 | 9.75 | 17.03 | 16.09 | 10.90 | 19.84 | 22.47 |
| ATA | 0.58 | 0.56 | 0.51 | 0.52 | 0.51 | 0.53 | 0.49 |
| ATC | 0.52 | 0.50 | 0.45 | 0.45 | 0.47 | 0.42 | 0.38 |
| ATG | 11.38 | 14.85 | 23.55 | 40.11 | 20.48 | 30.27 | 108.77 |
| ATT | 0.48 | 0.46 | 0.43 | 0.42 | 0.43 | 0.41 | 0.39 |
| CAA | 1.55 | 2.00 | 6.63 | 5.06 | 3.42 | 6.29 | 4.66 |
| CAC | 0.57 | 0.65 | 0.99 | 0.75 | 0.86 | 0.93 | 3.20 |
| CAG | 15.05 | 11.83 | 26.90 | 26.86 | 22.15 | 32.07 | 137.31 |
| CAT | 4.41 | 6.06 | 17.67 | 7.55 | 6.30 | 8.13 | 7.87 |
| CCA | 0.48 | 0.46 | 0.41 | 0.43 | 0.42 | 0.40 | 0.39 |
| CCC | 0.53 | 0.51 | 0.46 | 0.45 | 0.45 | 0.43 | 0.44 |
| CCG | 3.35 | 2.67 | 6.87 | 6.98 | 4.69 | 8.56 | 5.44 |
| CCT | 0.53 | 0.50 | 0.45 | 0.49 | 0.46 | 0.43 | 0.38 |
| CGA | 9.23 | 49.45 | 43.50 | 106.20 | 62.04 | 27.17 | 116.32 |
| CGC | 13.79 | 8.59 | 17.62 | 15.06 | 12.16 | 21.40 | 11.61 |
| CGG | 16.85 | 223.84 | 46.41 | 346.14 | 96.28 | 90.82 | 252.73 |
| CGT | 12.66 | 23.87 | 36.06 | 42.02 | 20.18 | 27.33 | 179.69 |
| CTA | 0.54 | 0.50 | 0.45 | 0.47 | 0.47 | 0.45 | 0.49 |
| CTC | 0.51 | 0.48 | 0.43 | 0.44 | 0.45 | 0.42 | 0.38 |
| CTG | 5.39 | 3.40 | 6.92 | 8.28 | 5.95 | 11.34 | 4.20 |
| CTT | 0.52 | 0.52 | 0.46 | 0.46 | 0.44 | 0.44 | 0.82 |
| GAA | 10.98 | 7.67 | 14.45 | 11.25 | 9.75 | 11.86 | 6.86 |
| GAC | 0.88 | 1.35 | 4.85 | 2.92 | 2.40 | 5.25 | 4.15 |
| GAG | 11.85 | 55.33 | 39.91 | 118.83 | 109.84 | 85.83 | 390.44 |
| GAT | 7.69 | 5.68 | 10.55 | 9.22 | 6.41 | 11.03 | 9.53 |
| GCA | 0.49 | 0.49 | 0.47 | 0.52 | 0.53 | 0.59 | 0.61 |
| GCC | 0.53 | 0.52 | 0.50 | 0.50 | 0.49 | 0.46 | 0.52 |
| GCG | 13.68 | 13.44 | 22.56 | 46.72 | 32.75 | 35.86 | 97.69 |
| GCT | 0.51 | 0.50 | 0.45 | 0.48 | 0.46 | 0.45 | 0.61 |
| GGA | 9.74 | 82.32 | 39.45 | 172.39 | 102.30 | 85.53 | 77.46 |
| GGC | 12.21 | 10.04 | 17.84 | 15.18 | 14.04 | 27.07 | 17.46 |
| GGG | 18.90 | 381.76 | 70.36 | 281.12 | 164.21 | 111.87 | 718.40 |
| GGT | 10.87 | 22.11 | 32.25 | 88.31 | 40.66 | 40.16 | 257.92 |
| GTA | 0.56 | 0.59 | 0.77 | 0.72 | 0.76 | 1.03 | 1.55 |
| GTC | 0.52 | 0.51 | 0.46 | 0.52 | 0.49 | 0.45 | 0.58 |
| GTG | 11.43 | 16.42 | 28.54 | 72.25 | 33.31 | 47.65 | 382.48 |
| GTT | 0.48 | 0.45 | 0.41 | 0.43 | 0.43 | 0.42 | 0.76 |
| TAA | 0.58 | 0.67 | 1.59 | 0.99 | 1.04 | 1.69 | 1.53 |
| TAC | 0.56 | 0.54 | 0.51 | 0.51 | 0.52 | 0.50 | 0.44 |
| TAG | 11.34 | 10.19 | 15.99 | 22.04 | 15.24 | 27.89 | 35.59 |
| TAT | 0.56 | 0.64 | 1.26 | 0.85 | 0.89 | 1.21 | 2.60 |
| TCA | 0.50 | 0.48 | 0.43 | 0.45 | 0.46 | 0.45 | 0.44 |
| TCC | 0.50 | 0.48 | 0.43 | 0.46 | 0.44 | 0.42 | 0.47 |
| TCG | 10.38 | 8.24 | 14.95 | 15.64 | 9.96 | 15.57 | 34.80 |
| TCT | 0.52 | 0.50 | 0.45 | 0.44 | 0.46 | 0.41 | 0.46 |
| TGA | 9.68 | 61.64 | 44.45 | 188.12 | 99.42 | 46.89 | 186.41 |
| TGC | 13.64 | 9.07 | 15.11 | 11.69 | 11.22 | 15.01 | 8.15 |
| TGG | 12.90 | 223.88 | 63.67 | 259.65 | 115.56 | 80.51 | 210.65 |
| TGT | 11.53 | 18.90 | 31.99 | 36.30 | 18.72 | 21.90 | 122.87 |
| TTA | 0.61 | 0.57 | 0.52 | 0.54 | 0.51 | 0.52 | 0.30 |
| TTC | 0.53 | 0.50 | 0.44 | 0.45 | 0.44 | 0.43 | 0.27 |
| TTG | 13.57 | 6.61 | 10.15 | 13.27 | 7.38 | 14.26 | 12.24 |
| TTT | 0.53 | 0.49 | 0.44 | 0.45 | 0.48 | 0.44 | 0.28 |

PAM Depletion Scores (xCas9v3.7-3.12 Mutations)

| | 22-68 | 22-75 | 22-78 | 22-82 | 22-87 | 22-90 |
|---|---|---|---|---|---|---|
| | xCas9 | xCas9 | xCas9 | xCas9 | xCas9 | xCas9 |
| | 3.7 | 3.8 | 3.9 | 3.10 | 3.11 | 3.12 |
| AAA | 0.92 | 1.00 | 4.30 | 0.63 | 0.48 | 1.67 |
| AAC | 8.72 | 3.59 | 1.55 | 1.59 | 0.94 | 1.34 |
| AAG | 23.44 | 20.16 | 16.46 | 10.63 | 26.46 | 22.44 |
| AAT | 1.03 | 0.89 | 1.22 | 1.07 | 0.76 | 0.96 |
| ACA | 0.80 | 0.92 | 0.52 | 0.68 | 1.09 | 0.83 |
| ACC | 0.42 | 1.37 | 0.32 | 1.03 | 0.81 | 0.53 |
| ACG | 11.20 | 6.65 | 21.80 | 1.38 | 1.23 | 0.88 |
| ACT | 0.27 | 0.38 | 0.24 | 0.88 | 0.54 | 0.66 |
| AGA | 11.63 | 11.95 | 14.60 | 0.89 | 1.42 | 1.78 |
| AGC | 1.03 | 11.25 | 8.05 | 1.54 | 1.77 | 2.39 |
| AGG | 8.05 | 11.61 | 21.28 | 1.37 | 33.28 | 113.98 |
| AGT | 11.10 | 6.85 | 12.73 | 0.39 | 2.70 | 0.91 |
| ATA | 0.73 | 0.79 | 0.58 | 1.04 | 0.70 | 0.94 |
| ATC | 0.43 | 0.47 | 0.28 | 0.63 | 0.86 | 0.87 |
| ATG | 15.46 | 13.03 | 4.76 | 2.21 | 2.39 | 1.73 |
| ATT | 0.62 | 0.30 | 0.53 | 0.59 | 0.91 | 0.74 |
| CAA | 8.96 | 6.08 | 1.97 | 1.46 | 0.53 | 0.67 |
| CAC | 1.65 | 1.31 | 0.81 | 0.60 | 0.75 | 0.73 |
| CAG | 3.46 | 1.99 | 1.44 | 23.50 | 9.51 | 13.76 |
| CAT | 20.52 | 44.57 | 18.70 | 16.29 | 15.96 | 0.36 |
| CCA | 0.40 | 0.31 | 0.64 | 0.87 | 0.55 | 0.64 |
| CCC | 0.35 | 0.54 | 0.55 | 1.12 | 0.56 | 0.69 |
| CCG | 4.35 | 5.28 | 5.57 | 1.37 | 0.94 | 0.8 |
| CCT | 0.40 | 1.04 | 0.36 | 1.22 | 0.67 | 0.59 |
| CGA | 4.47 | 7.60 | 5.26 | 1.35 | 1.36 | 2.02 |
| CGC | 13.35 | 10.98 | 5.45 | 4.03 | 5.37 | 3.66 |
| CGG | 6.06 | 5.52 | 25.23 | 31.85 | 19.44 | 34.65 |
| CGT | 17.13 | 7.41 | 17.31 | 0.63 | 1.44 | 0.66 |
| CTA | 0.76 | 0.50 | 0.50 | 1.97 | 1.45 | 1.05 |
| CTC | 0.40 | 0.26 | 0.65 | 0.58 | 0.45 | 0.81 |
| CTG | 5.64 | 9.49 | 6.82 | 1.35 | 1.41 | 2.11 |
| CTT | 0.50 | 0.63 | 0.68 | 0.54 | 0.73 | 0.88 |
| GAA | 9.81 | 6.07 | 4.68 | 2.07 | 0.87 | 1.09 |
| GAC | 3.91 | 3.95 | 2.82 | 0.44 | 0.86 | 0.56 |
| GAG | 23.26 | 21.32 | 9.88 | 16.93 | 18.42 | 40.73 |
| GAT | 5.06 | 9.85 | 1.07 | 0.75 | 1.06 | 0.63 |
| GCA | 0.56 | 0.81 | 0.79 | 1.46 | 0.44 | 0.89 |
| GCC | 0.35 | 0.52 | 0.71 | 0.65 | 3.67 | 0.73 |
| GCG | 16.11 | 12.53 | 9.43 | 1.59 | 1.98 | 2.22 |
| GCT | 0.42 | 0.88 | 0.27 | 0.66 | 1.14 | 0.76 |
| GGA | 3.01 | 18.40 | 33.07 | 27.28 | 16.20 | 46.82 |
| GGC | 6.03 | 3.83 | 11.69 | 8.03 | 22.56 | 3.92 |
| GGG | 12.16 | 10.26 | 15.08 | 17.30 | 25.85 | 128.03 |
| GGT | 11.25 | 13.77 | 2.94 | 12.71 | 16.11 | 16.22 |
| GTA | 1.38 | 1.08 | 1.24 | 0.75 | 3.52 | 1.09 |
| GTC | 0.96 | 0.31 | 0.50 | 0.93 | 0.72 | 0.81 |
| GTG | 6.68 | 17.93 | 26.86 | 9.93 | 9.61 | 2.72 |
| GTT | 0.45 | 0.33 | 0.99 | 0.65 | 1.61 | 1.01 |
| TAA | 3.04 | 2.16 | 1.84 | 1.21 | 1.08 | 0.68 |
| TAC | 0.61 | 0.52 | 0.68 | 0.95 | 0.68 | 0.86 |
| TAG | 4.76 | 15.71 | 1.10 | 8.15 | 8.25 | 8.87 |
| TAT | 1.62 | 2.10 | 1.01 | 0.73 | 0.74 | 0.66 |
| TCA | 0.41 | 0.66 | 0.58 | 0.46 | 0.77 | 0.68 |
| TCC | 0.54 | 0.29 | 0.38 | 1.36 | 0.86 | 0.82 |
| TCG | 1.41 | 4.20 | 9.11 | 0.84 | 0.46 | 0.69 |
| TCT | 0.47 | 0.44 | 0.38 | 0.60 | 1.00 | 0.94 |
| TGA | 22.42 | 5.39 | 15.72 | 1.78 | 3.13 | 3.69 |
| TGC | 6.78 | 6.81 | 11.35 | 2.4 | 6.55 | 4.08 |
| TGG | 20.36 | 11.36 | 11.56 | 9.97 | 21.64 | 19.87 |
| TGT | 10.32 | 8.82 | 21.17 | 0.94 | 0.79 | 0.73 |
| TTA | 0.65 | 0.29 | 0.77 | 0.94 | 1.35 | 0.75 |
| TTC | 0.62 | 1.07 | 0.55 | 0.86 | 1.07 | 0.58 |
| TTG | 8.41 | 6.07 | 9.07 | 0.67 | 0.56 | 0.72 |
| TTT | 0.43 | 0.56 | 0.44 | 0.68 | 0.60 | 0.75 |

### REFERENCES

1. Humbert O, Davis L, Maizels N. Targeted gene therapies: tools, applications, optimization. Crit Rev Biochem Mol. 2012; 47 (3): 264-81. PMID: 22530743.
2. Perez-Pinera P, Ousterout D G, Gersbach C A. Advances in targeted genome editing. Curr Opin Chem Biol. 2012; 16 (3-4): 268-77. PMID: 22819644.
3. Urnov F D, Rebar E J, Holmes M C, Zhang H S, Gregory P D. Genome editing with engineered zinc finger nucleases. Nat Rev Genet. 2010; 11 (9): 636-46. PMID: 20717154.
4. Joung J K, Sander J D. TALENs: a widely applicable technology for targeted genome editing. Nat Rev Mol Cell Biol. 2013; 14 (1): 49-55. PMID: 23169466.
5. Charpentier E, Doudna J A. Biotechnology: Rewriting a genome. Nature. 2013; 495, (7439): 50-1. PMID: 23467164.
6. Pan Y, Xia L, Li A S, Zhang X, Sirois P, Zhang J, Li K. Biological and biomedical applications of engineered nucleases. Mol Biotechnol. 2013; 55 (1): 54-62. PMID: 23089945.
7. De Souza, N. Primer: genome editing with engineered nucleases. Nat Methods. 2012; 9 (1): 27. PMID: 22312638.
8. Santiago Y, Chan E, Liu P Q, Orlando S, Zhang L, Urnov F D, Holmes M C, Guschin D, Waite A, Miller J C, Rebar E J, Gregory P D, Klug A, Collingwood T N. Targeted gene knockout in mammalian cells by using engineered zinc-finger nucleases. Proc Natl Acad Sci USA. 2008; 105 (15): 5809-14. PMID: 18359850.
9. Cargill M, Altshuler D, Ireland J, Sklar P, Ardlie K, Patil N, Lane C R, Lim E P, Kalyanaraman N, Nemesh J, Ziaugra L, Friedland L, Rolfe A, Warrington J, Lipshutz R, Daley G Q, Lander E S. Characterization of single-nucleotide polymorphisms in coding regions of human genes. Nat Genet. 1999; 22 (3): 231-8. PMID: 10391209.
10. Jansen R, van Embden J D, Gaastra W, Schouls L M. Identification of genes that are associated with DNA repeats in prokaryotes. Mol Microbiol. 2002; 43 (6): 1565-75. PMID: 11952905.
11. Mali P, Esvelt K M, Church G M. Cas9 as a versatile tool for engineering biology. Nat Methods. 2013; 10 (10): 957-63. PMID: 24076990.
12. Jore M M, Lundgren M, van Duijin E, Bultema J B, Westra E R, Waghmare S P, Wiedenheft B, Pul U, Wurm R, Wagner R, Beijer M R, Barendregt A, Shou K, Snijders A P, Dickman M J, Doudna J A, Bockema E J, Heck A J, van der Oost J, Brouns S J. Structural basis for CRISPR RNA-guided DNA recognition by Cascade. Nat Struct Mol Biol. 2011; 18 (5): 529-36. PMID: 21460843.
13. Horvath P, Barrangou R. CRISPR/Cas, the immune system of bacteria and archaea. Science. 2010; 327 (5962): 167-70. PMID: 20056882.
14. Wiedenheft B, Sternberg S H, Doudna J A. RNA-guided genetic silencing systems in bacteria and archaea. Nature. 2012; 482 (7385): 331-8. PMID: 22337052.
15. Gasiunas G, Siksnys V. RNA-dependent DNA endonuclease Cas9 of the CRISPR system: Holy Grail of genome editing? Trends Microbiol. 2013; 21 (11): 562-7. PMID: 24095303.
16. Qi L S, Larson M H, Gilbert L A, Doudna J A, Weissman J S, Arkin A P, Lim W A. Repurposing CRISPR as an RNA-guided platform for sequence-specific control of gene expression. Cell. 2013; 152 (5): 1173-83. PMID: 23452860.
17. Perez-Pinera P, Kocak D D, Vockley C M, Adler A F, Kabadi A M, Polstein L R, Thakore P I, Glass K A, Ousterout D G, Leong K W, Guilak F, Crawford G E, Reddy T E, Gersbach C A. RNA-guided gene activation by CRISPR-Cas9-based transcription factors. Nat Methods. 2013; 10 (10): 973-6. PMID: 23892895.
18. Mali P, Aach J, Stranges P B, Esvelt K M, Moosburner M, Kosuri S, Yang L, Church G M. CAS9 transcriptional activators for target specificity screening and paired nickases for cooperative genome engineering. Nat Biotechnol. 2013; 31 (9): 833-8. PMID: 23907171.
19. Gilbert L A, Larson M H, Morsut L, Liu Z, Brar G A, Torres S E, Stern-Ginossar N, Brandman O, Whitehead E H, Doudna J A, Lim W A, Weissman J S, Qi L S. CRISPR-mediated modular RNA-guided regulation of transcription in eukaryotes. Cell. 2013; 154 (2): 442-51. PMID: 23849981.
20. Larson M H, Gilbert L A, Wang X, Lim W A, Weissman J S, Qi L S. CRISPR interference (CRISPRi) for sequence-specific control of gene expression. Nat Protoc. 2013; 8 (11): 2180-96. PMID: 24136345.
21. Mali P, Yang L, Esvelt K M, Aach J, Guell M, DiCarlo J E, Norville J E, Church G M. RNA-guided human genome engineering via Cas9. Science. 2013; 339 (6121): 823-6. PMID: 23287722.
22. Cole-Strauss A, Yoon K, Xiang Y, Byrne B C, Rice M C, Gryn J, Holloman W K, Kmicc E B. Correction of the mutation responsible for sickle cell anemia by an RNA-DNA oligonucleotide. Science. 1996; 273 (5280): 1386-9. PMID: 8703073.
23. Tagalakis A D, Owen J S, Simons J P. Lack of RNA-DNA oligonucleotide (chimeraplast) mutagenic activity in mouse embryos. Mol Reprod Dev. 2005; 71 (2): 140-4. PMID: 15791601.
24. Ray A, Langer M. Homologous recombination: ends as the means. Trends Plant Sci. 2002; 7 (10): 435-40. PMID 12399177.
25. Britt A B, May G D. Re-engineering plant gene targeting. Trends Plant Sci. 2003; 8 (2): 90-5. PMID: 12597876.
26. Vagner V, Ehrlich S D. Efficiency of homologous DNA recombination varies along the Bacillus subtilis chromosome. J Bacteriol. 1988; 170 (9): 3978-82. PMID: 3137211.
27. Salch-Gohari N, Helleday T. Conservative homologous recombination preferentially repairs DNA double-strand breaks in the S phase of the cell cycle in human cells. Nucleic Acids Res. 2004; 32 (12): 3683-8. PMID: 15252152.
28. Lombardo A, Genovese P, Beausejour C M, Colleoni S, Lec Y L, Kim K A, Ando D, Urnov F D, Galli C, Gregory P D, Holmes M C, Naldini L. Gene editing in human stem cells using zinc finger nucleases and integrase-defective lentiviral vector delivery. Nat Biotechnol. 2007; 25 (11): 1298-306. PMID: 17965707.
29. Conticello S G. The AID/APOBEC family of nucleic acid mutators. Genome Biol. 2008; 9 (6): 229. PMID: 18598372.
30. Reynaud C A, Aoufouchi S, Faili A, Weill J C. What role for AID: mutator, or assembler of the immunoglobulin mutasome? Nat Immunol. 2003; 4 (7): 631-8.
31. Bhagwat A S. DNA-cytosine deaminases: from antibody maturation to antiviral defense. DNA Repair (Amst). 2004; 3 (1): 85-9. PMID: 14697763.
32. Navaratnam N, Sarwar R. An overview of cytidine deaminases. Int J Hematol. 2006; 83 (3): 195-200. PMID: 16720547.
33. Holden L G, Prochnow C, Chang Y P, Bransteitter R, Chelico L, Sen U, Stevens R C, Goodman M F, Chen X S. Crystal structure of the anti-viral APOBEC3G catalytic domain and functional implications. Nature. 2008; 456 (7218): 121-4. PMID: 18849968.
34. Chelico L, Pham P, Petruska J, Goodman M F. Biochemical basis of immunological and retroviral responses to DNA-targeted cytosine deamination by activation-induced cytidine deaminase and APOBEC3G. J Biol Chem. 2009; 284 (41). 27761-5. PMID: 19684020.
35. Pham P, Bransteitter R, Goodman M F. Reward versus risk: DNA cytidine deaminases triggering immunity and disease. Biochemistry. 2005; 44 (8): 2703-15. PMID 15723516.
36. Chen X, Zaro J L, Shen W C. Fusion protein linkers: property, design and functionality. Adv Drug Deliv Rev. 2013; 65 (10): 1357-69. PMID: 23026637.
37. Slaymaker, I. M. et al. Rationally engineered Cas9 nucleases with improved specificity. Science, doi: 10.1126/science.aad5227 (2015).
38. Kleinstiver, B. P. et al. Engineered CRISPR-Cas9 nucleases with altered PAM specificities. Nature 523, 481-485, doi: 10.1038/nature14592 (2015).
39. Pattanayak, V. et al. High-throughput profiling of off-target DNA cleavage reveals RNA-programmed Cas9 nuclease specificity. Nature Biotechnology 31, 839-843, doi: 10.1038/nbt.2673 (2013).
40. Shcherbakova, D. M. & Verkhusha, V. V. Near-infrared fluorescent proteins for multicolor in vivo imaging. Nature Methods 10, 751-754, doi: 10.1038/nmeth.2521 (2013).
41. Kleinstiver, et al., High-fidelity CRISPR-Cas9 nucleases with no detectable genome-wide off-target effects. Nature 529, 490-495 doi: 10.1038/nature16526 (2016).

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

1. A Cas9 protein comprising an amino acid sequence that is at least 80% identical to the amino acid sequence of a Cas9 protein as provided by any one of SEQ ID NOs: 9-262,
wherein the amino acid sequence of the Cas9 protein comprises at least one mutation in an amino acid residue selected from the group consisting of amino acid residues 267, 294, 405, 480, 543, 1219, 1224, 1256, and 1362 of the amino acid sequence provided in SEQ ID NO: 9, or in a corresponding amino acid residue in any of the amino acid sequences provided in SEQ ID NOS: 10-262, and
wherein the amino acid sequence of the Cas9 protein is not identical to the amino acid sequence of a naturally occurring Cas9 protein.

2-434. (canceled)

435. The Cas9 protein of claim 1, wherein the Cas9 protein comprises an amino acid sequence that is at least 90% identical to the amino acid sequence of SEQ ID NO: 9, or a corresponding mutation in any of the amino acid sequences provided in SEQ ID NOs: 10-262.

436. The Cas9 protein of claim 1, wherein the amino acid sequence of the Cas9 protein comprises at least one mutation selected from the group consisting of S267G, K294R, F405I, E480K, E543D, E1219V, N1224K, Q1256K, and L1362P in the amino acid sequence provided in SEQ ID NO: 9, or a corresponding mutation in any of the amino acid sequences provided in SEQ ID NOs: 10-262.

437. The Cas9 protein of claim 1, wherein the amino acid sequence of the Cas9 protein comprises a K294R mutation in the amino acid sequence provided in SEQ ID NO: 9 and further comprises at least one mutation selected from the group consisting of S267G, F405I, E480K, E543D, E1219V, N1224K, Q1256K, and L1362P in the amino acid sequence provided in SEQ ID NO: 9, or a corresponding mutation in any of the amino acid sequences provided in SEQ ID NOS: 10-262.

438. The Cas9 protein of claim 1, wherein the amino acid sequence of the Cas9 protein comprises any one of the following groups of mutations:
K294R, E480K, E543D, E1219V, Q1256K, and L1362P;
K294R, E480K, E543D, E1219V, and Q1256K;
S267G, K294R, E480K, E543D, E1219V, N1224K, and Q1256K;
R115H, K141Q, S267G, K294R, Q394H, E480K, E543D, E1219V, and Q1256K;
S267G, K294R, E480K, E543D, E1219V, and Q1256K;
D257N, S267G, K294R, T466A, E480K, E543D, 11063V, E1219V, and Q1256K;
K294R, E480K, E543D, A711E, E1219V, and Q1256K;
P230S, S267G, K294R, E480K, E543D, E1219V, and Q1256K;
P230F, S267G, K294R, E480K, E543D, E1219V, and Q1256K;
K294R, E480K, E543D, A711E, E1207G, E1219V, and Q1256K; or
N175T, S267G, K294R, E480K, E543D, E1219V, and Q1256K;
in the amino acid sequence provided in SEQ ID NO: 9, or a corresponding mutation in any of the amino acid sequences provided in SEQ ID NOs: 10-262.

439. The Cas9 protein of claim 1, wherein the Cas9 protein comprises a D10A and/or a H840A mutation of the amino acid sequence provided in SEQ ID NO: 9, or a corresponding mutation in any of the amino acid sequences provided in SEQ ID NOs: 10-262.

440. The Cas9 protein of claim 1, wherein the Cas9 protein recognizes a non-canonical PAM sequence.

441. The Cas9 protein of claim 440, wherein the non-canonical PAM sequence is selected from the group consisting of AAA, AAC, AAG, AAT, CAA, CAC, CAG, CAT, GAA, GAC, GAG, GAT, TAA, TAC, TAG, TAT, ACA, ACC, ACG, ACT, CCA, CCC, CCG, CCT, GCA, GCC, GCG, GCT, TCA, TCC, TCG, TCT, AGA, AGC, AGT, CGA, CGC, CGT, GGA, GGC, GGT, TGA, TGC, TGT, ATA, ATC, ATG, ATT, CTA, CTC, CTG, CTT, GTA, GTC, GTG, GTT, TTA, TTC, TTG, and TTT.

442. A fusion protein comprising the Cas9 protein of claim 1, wherein the Cas9 protein is fused to an effector domain.

443. The fusion protein of claim 442, wherein the effector domain comprises an enzyme domain.

444. The fusion protein of claim 442, wherein the effector domain comprises a nuclease domain, a nickase domain, a recombinase domain, a deaminase domain, a methyltransferase domain, a methylase domain, an acetylase domain, an acetyltransferase domain, a transcriptional activator domain, or a transcriptional repressor domain.

445. The fusion protein of claim 442, wherein the effector domain is a nuclease domain.

446. A dimer of the fusion protein of claim 445.

447. A fusion protein comprising a first Cas9 protein fused to a second Cas9 protein, wherein the first Cas9 protein is the Cas9 protein of claim 1.

448. A complex comprising the Cas9 protein of claim 1 and a guide RNA bound to the Cas9 protein.

449. A complex comprising the fusion protein of claim 447,
a first guide RNA bound to the first Cas9 protein of the fusion protein, and
a second guide RNA bound to the second Cas9 protein of the fusion protein.

450. A method comprising contacting a DNA molecule with the Cas9 protein of claim 1 and a guide RNA, wherein the guide RNA is about 15-100 nucleotides long and comprises a sequence of at least 10 contiguous nucleotides that is complementary to a target sequence.

451. A polynucleotide encoding the Cas9 protein of claim 1.

452. A vector comprising the polynucleotide of claim 451.

453. A cell comprising the Cas9 protein of claim 1, wherein the cell is an isolated cell.
